(19) United States
(12) Patent Application Publication
(10) Pub. No.: US 2018/0356432 A1
(43) Pub. Date: Dec. 13, 2018

(54) MARKERS FOR CORONARY ARTERY DISEASE AND USES THEREOF

(71) Applicant: CardioDx, Inc., Redwood City, CA (US)

(72) Inventors: Philip BEINEKE, Mountain View, CA (US); James A. WINGROVE, Sunnyvale, CA (US); Karen FITCH, Mountain View, CA (US); Steven ROSENBERG, Oakland, CA (US); Andrea M. JOHNSON, Palo Alto, CA (US)

(21) Appl. No.: 15/756,430

(22) PCT Filed: Aug. 31, 2016
(86) PCT No.: PCT/US16/49717

§ 371 Date: Feb. 28, 2018

Related U.S. Application Data
(60) Provisional application No. 62/212,935, filed on Sep. 1, 2015.

(51) Int. Cl. G01N 33/68; G06F 19/12; G16H 50/30; G16H 10/40

(52) U.S. Cl. CPC G01N 33/6893; G06F 19/12; G16H 50/30; G16H 10/40; G01N 2333/4727; G01N 2800/50; G01N 2333/775; G01N 2333/948; G01N 2333/575; G01N 2800/324

(57) ABSTRACT

Markers and methods useful for assessing coronary artery disease in a subject are provided, along with related kits, systems, and media. Also provided are predictive models, based on the markers, as well as computer systems, and software embodiments of the models for scoring and optionally classifying samples.

### CROSS REFERENCE TO RELATED APPLICATIONS

This application claims the benefit of U.S. Provisional Application No. 62/212,935, filed Sep. 1, 2015, which is hereby incorporated by reference, in its entirety, for all purposes.

### BACKGROUND

The determination of the underlying etiology of symptoms suggestive of obstructive coronary artery disease (obstructive CAD, >70% stenosis in a major coronary artery, by clinical read) is a common clinical challenge in both primary care and cardiology clinics. Usual care in low to medium risk patients often involves a family history, risk factor assessment, followed by stress testing with or without non-invasive imaging. If positive, this is often followed by invasive coronary angiography (ICA). Despite extensive adoption of this usual care paradigm, more than 60% of patients referred for angiography do not have obstructive CAD. The development of novel diagnostic tests may identify symptomatic patients without obstructive CAD, allowing the patient to avoid subsequent cardiac testing and the clinicians to look elsewhere for the cause of their symptoms.

Previous work has demonstrated that peripheral blood gene expression profiling can be used to determine the likelihood of obstructive CAD in symptomatic patients (e.g., Corus; see related, co-owned patents including U.S. Pat. Nos. 9,122,777 and 8,914,240, each of which is herein incorporated by reference, in its entirety, for all purposes). Peripheral blood gene expression is typically limited at present to interrogating the changes in gene expression within circulating cells of the immune system due to the interaction of the cells with the diseased tissue. In addition, gene expression-based assays can be expensive to utilize and can be difficult to implement in a clinical lab setting, which can limit the placement of such assays in those settings.

The various limitations of a gene expression-based approach are overcome or minimized by the various approaches described herein, e.g., by instead utilizing an approach that includes protein-based expression data. Proteins, which can be released into circulation in response to CAD, may capture a more direct response to CAD, e.g., the proteins are released directly from the diseased site, or a more systemic reflection of the disease, e.g., the proteins are released from multiple tissues or organs affected by CAD. In addition, protein-based assays can be more cost effective than gene expression based assays, and are generally easier to implement in the clinical lab setting, thus expanding the potential placement of such assays in those facilities. Finally, certain approaches taken herein have been demonstrated in varying head-to-head studies in the Examples described herein to have better performance and to be more predictive of CAD relative to Corus, e.g., as measured using area under the curve (AUC).

### BRIEF DESCRIPTION OF THE SEVERAL VIEWS OF THE DRAWINGS

The patent or application file contains at least one drawing executed in color. Copies of this patent or patent application publication with color drawings will be provided by the U.S. Patent and Trademark Office upon request and payment of the necessary fee. These and other features, aspects, and advantages of the present invention will become better understood with regard to the following description, and accompanying drawings, where:

FIG. 1A shows an assessment of the correlation between top Phase 1 markers; overall, pairwise correlation was low (r<0.7). The color key begins dark for 0 and ends light for 1 (left to right).

FIG. 1B shows distribution of percent stenosis across genders and age groups.

FIG. 2 shows marginal distributions of protein markers (log transformed, centered and scaled values).

FIG. 3 shows rank correlations among pairs of predictor variables.

FIG. 4 shows a cluster diagram of the Spearman's non-parametric measure of correlation among the quantitative variables in the models.

FIG. 5 shows the median AIC and corrected AIC values for main models M1 through M11.

FIG. 6 shows estimated odds ratios for all markers using Model 7.

FIG. 7 shows estimates of AUC (area under the curve) values for all main models.

FIG. 8 shows plots of the ROC curves for the best proteomics model and the Corus scores on the CADP2 patients (left; AUC for Model 7 is 0.811 and AUC for Corus is 0.770) and on the same set, after excluding Corus Alg. Dev. Subject (right; AUC for Model 7 is 0.832 and AUC for Corus is 0.768). Model 7 is sold line and Corus is dashed line.

FIG. 9 shows relative diagnostic performance measures for Model 7 on CADP2 patients for two cutoffs, compared to performance of Corus on the same patients (Corus.c15), and the published values for Corus in the COMPASS and PREDICT studies.

FIG. 10 shows ROC plots comparing the predictive performance of Model 7 and Corm within different subsets of subjects. Model 7 is sold line and Corus is dashed line. For each graph the AUC for Model 7 is on the top and the AUC for Corus is on the bottom.

FIG. 11 shows odds ratio estimates for the terms in the exploratory models in Exp1.1 to Exp1.13. The odds ratio for gender is not shown for platting purposes, due to its large size, relative to the other OR (odds ratio).

FIG. 12 shows odds ratio (OR) estimates for the terms in the exploratory models Exp2.1 to Exp2.3. Note that this data set excludes the Alg Dev subjects.

FIG. 13 shows a comparison of predicted values from Model 7 to the percent stenosis of the same patient. Points are colored by agreement of model 7 to reference status.

FIG. 14 shows comparison of predicted values from Model 7 to the percent stenosis of the same patient. Points are colored by agreement of model 7 to reference status.

FIG. 15 shows comparison of predicted values from Model 7 to predicted values from the Corus score run on the same sample. Points are colored by true reference status. The dashed lines indicate the cutoff of 20% for Model 7 and the cutoff of 15 for Corus.

FIG. 16 shows the ability of the model to explain the variation in the data (corrected AIC) compared to the ability of the model to correctly classify the patients for obstructive CAD (AUC) for the number of markers in the model (moving sequentially from 15 to 1 markers).

### SUMMARY

Described herein is a method for determining coronary artery disease risk in a subject, comprising: performing or having performed at least one protein detection assay on a sample from the subject to generate a dataset comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6; and generating or having generated, by a computer processor, a score indicative of coronary artery disease (CAD) risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD.

Also disclosed herein is a method for determining coronary artery disease risk in a subject, comprising: obtaining or having obtained a dataset associated with a sample from the subject comprising data representing protein expression levels to at least two markers comprising corin, APOB, HSP70, RBP4, SERPINA12, NTproBNP, PIGF, adiponectin, APOA1, S100A8, MPO, S100A12, or TNFAIP6; generating or having generated, by a computer processor, a score indicative of coronary artery disease (CAD) risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD.

Also disclosed herein is a method for generating a dataset comprising data representing protein expression levels for a subject that has CAD or is suspected of having CAD, comprising: obtaining or having obtained a sample from the subject, wherein the subject has CAD or is suspected of having CAD; performing or having performed at least one protein detection assay on the sample to generate a dataset comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6. In some aspects, the method further comprises generating, by a computer processor, a score indicative of coronary artery disease (CAD) risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD. In some aspects, the at least one protein detection assay is an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, amd immunoprecipitation.

In some aspects, the at least one protein detection assay is at least one enzyme-linked immunosorbent assay (ELISA), wherein the dataset comprises data representing expression levels corresponding to at least five markers comprising corin, APOB, HSP70, RBP4, SERPINA12, NTproBNP, PIGF, adiponectin, APOA1, S100A8, MPO, S100A12, or TNFAIP6, and wherein the score is more predictive of CAD than a score produced using Corus with the sample as measured using AIC or AUC. In some aspects, the dataset comprises data representing expression levels corresponding to at least three, four, or five markers comprising corin, APOB, HSP70, RBP4, SERPINA12, NTproBNP, PIGF, adiponectin, APOA1, S100A8, MPO, S100A12, or TNFAIP6. In some aspects, the dataset comprises data representing expression levels corresponding to at least three, four, or five markers comprising APOB, HSP70, SERPINA12, NTproBNP, PIGF, adiponectin, APOA1, S100A8, MPO, S100A12, or TNFAIP6.

In some aspects, a method disclosed herein further comprises classifying a sample according to the score. In some aspects, a method disclosed herein further comprises rating CAD risk using the score.

In some aspects, a sample comprises protein extracted from the blood of the subject.

In some aspects, the mathematical combination is based on a predictive model, optionally wherein the predictive model is a partial least squares model, a logistic regression model, a linear regression model, a linear discriminant analysis model, a ridge regression model, or a tree-based recursive partitioning model.

In some aspects, CAD is obstructive CAD.

In some aspects, method performance is characterized by an area under the curve (AUC) ranging from 0.52 to 0.81, 0.50 to 0.99, 0.55 to 0.65, 0.50 to 0.70, 0.70 to 0.79, 0.80 to 0.89, or 0.90 to 0.99. In some aspects, method performance is characterized by an area under the curve (AUC) ranging of at least 0.5, 0.52, 0.6, 0.7, 0.8, or 0.81.

In some aspects, a method disclosed herein further comprises obtaining data representing at least one clinical factor associated with the subject, optionally wherein the clinical factor comprises age of the subject and/or gender of the subject, and optionally mathematically combining the data representing the at least one clinical factor with the data representing the protein expression levels to generate the score. In some aspects, a method disclosed herein further comprises obtaining data representing at least one clinical factor associated with the subject, wherein the at least one clinical factor comprises at least one of age and gender. In some aspects, a method disclosed herein further comprises obtaining data representing at least one clinical factor associated with the subject, wherein the at least one clinical factor comprises age and gender. In some aspects, a method disclosed herein further comprises mathematically combining the data representing the at least one clinical factor with the data representing the protein expression levels to generate the score.

In some aspects, a subject is human.

In some aspects, an at least one protein detection assay is an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, amd immunoprecipitation.

In some aspects, a method disclosed herein further comprises taking at least one action based on the score, optionally wherein the at least one action comprises treating the subject, advising lifestyle changes to the subject, performing a procedure on the subject, performing further diagnostics on the subject, assessing the subject's health further, optimizing medical therapy, investigating non-cardiac etiologies of symptoms, or performing angiography on the subject.

In some aspects, obtaining the dataset comprises obtaining the sample and processing the sample to experimentally determine the dataset. In some aspects, obtaining the dataset comprises performing at least one protein detection assay, optionally wherein the at least one protein detection assay is an immunoassay, a protein-binding assay, an antibody-based assay, an antigen-binding protein-based assay, a protein-based array, ELISA, flow cytometry, a blot, or mass spectrometry. In some aspects, the at least one protein detection assay is an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, amd immunoprecipitation. In some aspects, obtaining the dataset comprises receiving the dataset from a third party that has processed the sample to experimentally determine the dataset.

Also disclosed herein is a system for determining coronary artery disease risk in a subject, comprising: a storage memory for storing a dataset associated with a sample from the subject comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6; and a processor communicatively coupled to the storage memory for generating a score indicative of CAD risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD.

In some aspects, the dataset comprises data representing expression levels corresponding to at least five markers comprising corin, APOB, HSP70, RBP4, SERPINA12, NTproBNP, PIGF, adiponectin, APOA1, S100A8, MPO, S100A12, or TNFAIP6, and wherein the score is more predictive of CAD than a score produced using Corus with the sample as measured using AIC or AUC. In some aspects, the dataset comprises data representing expression levels corresponding to at least three, four, or five markers comprising corin, APOB, HSP70, RBP4, SERPINA12, NTproBNP, PIGF, adiponectin, APOA1, S100A8, MPO, S100A12, or TNFAIP6. In some aspects, the dataset comprises data representing expression levels corresponding to at least three, four, or five markers comprising APOB, HSP70, SERPINA12, NTproBNP, PIGF, adiponectin, APOA1, S100A8, MPO, S100A12, or TNFAIP6.

In some aspects, a system further comprises code for classifying the sample according to the score. In some aspects, a system further comprises code for rating CAD risk using the score.

In some aspects, the sample comprises protein extracted from the blood of the subject.

In some aspects, the mathematical combination is based on a predictive model, optionally wherein the predictive model is a partial least squares model, a logistic regression model, a linear regression model, a linear discriminant analysis model, a ridge regression model, or a tree-based recursive partitioning model.

In some aspects, CAD is obstructive CAD. In some aspects, a subject is human.

In some aspects, performance of the mathematical combination is characterized by an area under the curve (AUC) ranging from 0.52 to 0.81, 0.50 to 0.99, 0.55 to 0.65, 0.50 to 0.70, 0.70 to 0.79, 0.80 to 0.89, or 0.90 to 0.99. In some aspects, performance of the mathematical combination is characterized by an area under the curve (AUC) ranging of at least 0.5, 0.52, 0.6, 0.7, 0.8, or 0.81.

In some aspects, a system further comprises a storage memory comprising data representing at least one clinical factor associated with the subject, optionally wherein the clinical factor comprises age of the subject and/or gender of the subject. In some aspects, the system further comprises a storage memory comprising data representing at least one clinical factor associated with the subject, wherein the at least one clinical factor comprises at least one of age and gender. In some aspects, the system further comprises a storage memory comprising data representing at least one clinical factor associated with the subject, wherein the at least one clinical factor comprises age and gender. In some aspects, the system further comprises a processor communicatively coupled to the storage memory for mathematically combining the data representing the at least one clinical factor with the data representing the protein expression levels to generate the score.

In some aspects, a system further comprises an apparatus for providing a readout that provides instructions for taking at least one action based on the score, optionally wherein the at least one action comprises treating the subject, advising lifestyle changes to the subject, performing a procedure on the subject, performing further diagnostics on the subject, assessing the subject's health further, optimizing medical therapy, investigating non-cardiac etiologies of symptoms, or performing angiography on the subject.

Also disclosed herein is a computer-readable storage medium storing computer-executable program code for determining coronary artery disease risk in a subject, comprising: program code for storing a dataset associated with a sample from the subject comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6; and program code for generating a score indicative of CAD risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD.

In some aspects, the dataset comprises data representing expression levels corresponding to at least five markers comprising corin, APOB, HSP70, RBP4, SERPINA12, NTproBNP, PIGF, adiponectin, APOA1, S100A8, MPO, S100A12, or TNFAIP6, and wherein the score is more predictive of CAD than a score produced using Corus with the sample as measured using AIC or AUC. In some aspects, the dataset comprises data representing expression levels corresponding to at least three, four, or five markers comprising corin, APOB, HSP70, RBP4, SERPINA12, NTproBNP, PIGF, adiponectin, APOA1, S100A8, MPO, S100A12, or TNFAIP6. In some aspects, the dataset comprises data representing expression levels corresponding to at least three, four, or five markers comprising APOB, HSP70, SERPINA12, NTproBNP, PIGF, adiponectin, APOA1, S100A8, MPO, S100A12, or TNFAIP6.

In some aspects, a medium further comprises program code for classifying the sample according to the score. In some aspects, a medium further comprises program code for rating CAD risk using the score.

In some aspects, a sample comprises protein extracted from the blood of the subject.

In some aspects, the mathematical combination is based on a predictive model, optionally wherein the predictive model is a partial least squares model, a logistic regression model, a linear regression model, a linear discriminant analysis model, a ridge regression model, or a tree-based recursive partitioning model.

In some aspects, CAD is obstructive CAD. In some aspects, a subject is human.

In some aspects, performance of the mathematical combination is characterized by an area under the curve (AUC) ranging from 0.52 to 0.81, 0.50 to 0.99, 0.55 to 0.65, 0.50 to 0.70, 0.70 to 0.79, 0.80 to 0.89, or 0.90 to 0.99. In some aspects, performance of the mathematical combination is characterized by an area under the curve (AUC) ranging of at least 0.5, 0.52, 0.6, 0.7, 0.8, or 0.81.

In some aspects, a medium further comprises program code for storing data representing at least one clinical factor associated with the subject, optionally wherein the clinical factor comprises age of the subject and/or gender of the subject. In some aspects, the medium further comprises program code for storing data representing at least one clinical factor associated with the subject, wherein the at least one clinical factor comprises at least one of age and gender. In some aspects, the medium further comprises program code for storing data representing at least one clinical factor associated with the subject, wherein the at least one clinical factor comprises age and gender. In some aspects, the medium further comprises program code for storing for mathematically combining the data representing the at least one clinical factor with the data representing the protein expression levels to generate the score.

In some aspects, a medium further comprises program code for storing instructions for taking at least one action based on the score, optionally wherein the at least one action comprises treating the subject, advising lifestyle changes to the subject, performing a procedure on the subject, performing further diagnostics on the subject, assessing the subject's health further, optimizing medical therapy, investigating non-cardiac etiologies of symptoms, or performing angiography on the subject.

Also disclosed herein is a kit for determining coronary artery disease risk in a subject, comprising: a set of reagents for generating a dataset via at least one protein detection assay that is associated with a sample from the subject comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6; and instructions for generating a score indicative of CAD risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD.

In some aspects, the at least one protein detection assay is at least one enzyme-linked immunosorbent assay (ELISA), wherein the dataset comprises data representing expression levels corresponding to at least five markers comprising corin, APOB, HSP70, RBP4, SERPINA12, NTproBNP, PIGF, adiponectin, APOA1, S100A8, MPO, S100A12, or TNFAIP6, and wherein the score is more predictive of CAD than a score produced using Corus with the sample as measured using AIC or AUC. In some aspects, the dataset comprises data representing expression levels corresponding to at least three, four, or five markers comprising corin, APOB, HSP70, RBP4, SERPINA12, NTproBNP, PIGF, adiponectin, APOA1, S100A8, MPO, S100A12, or TNFAIP6. In some aspects, the dataset comprises data representing expression levels corresponding to at least three, four, or five markers comprising APOB, HSP70, SERPINA12, NTproBNP, PIGF, adiponectin, APOA1, S100A8, MPO, S100A12, or TNFAIP6.

In some aspects, a kit further comprises instructions for classifying the sample according to the score. In some aspects, a kit further comprises instructions for rating CAD risk using the score.

In some aspects, a sample comprises protein extracted from the blood of the subject.

In some aspects, the mathematical combination is based on a predictive model, optionally wherein the predictive model is a partial least squares model, a logistic regression model, a linear regression model, a linear discriminant analysis model, a ridge regression model, or a tree-based recursive partitioning model.

In some aspects, CAD is obstructive CAD. In some aspects, a subject is human.

In some aspects, performance of the instructions for generating the score is characterized by an area under the curve (AUC) ranging from 0.52 to 0.81, 0.50 to 0.99, 0.55 to 0.65, 0.50 to 0.70, 0.70 to 0.79, 0.80 to 0.89, or 0.90 to 0.99. In some aspects, performance of the instructions for generating the score is characterized by an area under the curve (AUC) ranging of at least 0.5, 0.52, 0.6, 0.7, 0.8, or 0.81.

In some aspects, a kit further comprises instructions for obtaining data representing at least one clinical factor associated with the subject, optionally wherein the clinical factor comprises age of the subject and/or gender of the subject, and optionally comprising instructions for mathematically combining the data representing the at least one clinical factor with the data representing the protein expression levels to generate the score. In some aspects, the kit further comprises instructions for obtaining data representing at least one clinical factor associated with the subject, wherein the at least one clinical factor comprises at least one of age and gender. In some aspects, the kit further comprises instructions for obtaining data representing at least one clinical factor associated with the subject, wherein the at least one clinical factor comprises age and gender. In some aspects, the kit further comprises instructions for mathematically combining the data representing the at least one clinical factor with the data representing the protein expression levels to generate the score.

In some aspects, the at least one protein detection assay is an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, amd immunoprecipitation.

In some aspects, the reagents comprise one or more antibodies that bind to one or more of the markers, optionally wherein the antibodies are monoclonal antibodies or polyclonal antibodies.

In some aspects, a kit further comprises instructions for taking at least one action based on the score, optionally wherein the at least one action comprises treating the subject, advising lifestyle changes to the subject, performing a procedure on the subject, performing further diagnostics on the subject, assessing the subject's health further, optimizing medical therapy, investigating non-cardiac etiologies of symptoms, or performing angiography on the subject.

### DETAILED DESCRIPTION

Circulating proteins are well-established as biomarkers of disease. 137 protein biomarkers were interrogated for association with coronary artery disease, and subsequently a multi-analyte predictive model utilizing a subset of markers was created. The identification of biomarkers associated with the likelihood of coronary artery disease and creation of a predictive model could lead, e.g., to better patient stratification for further cardiovascular workup and intervention. Models to assist in determining the likelihood of coronary artery disease in a subject based on proteins markers were developed and tested. These models have been demonstrated to have greater predictive value for the likelihood of coronary artery disease relative to earlier coronary artery disease tests, including Cons.

Terms used in the claims and specification are defined as set forth below unless otherwise specified.

It must be noted that, as used in the specification and the appended claims, the singular forms “a,” “an” and “the” include plural referents unless the context clearly dictates otherwise.

A “subject” in the context of the present teachings is generally a mammal, e.g., a human. The subject can be a human patient, e.g., a human heart failure patient. The term “mammal” as used herein includes but is not limited to a human, non-human primate, dog, cat, mouse, rat, cow, horse, and pig. Mammals other than humans can be advantageously used as subjects that represent animal models of, e.g., heart failure. A subject can be male or female. A subject can be one who has been previously diagnosed or identified as having coronary artery disease. A subject can be one who has already undergone, or is undergoing, a therapeutic intervention for coronary artery disease. A subject can also be one who has not been previously diagnosed as having coronary artery disease; e.g., a subject can be one who exhibits one or more symptoms or risk factors for coronary artery disease, or a subject who does not exhibit symptoms or risk factors for coronary artery disease, or a subject who is asymptomatic for coronary artery disease.

A “sample” in the context of the present teachings refers to any biological sample that is isolated from a subject. A sample can include, without limitation, a single cell or multiple cells, fragments of cells, an aliquot of body fluid, whole blood, platelets, serum, plasma, red blood cells, white blood cells or leucocytes, endothelial cells, tissue biopsies, synovial fluid, lymphatic fluid, ascites fluid, and interstitial or extracellular fluid. The term “sample” also encompasses the fluid in spaces between cells, including gingival crevicular fluid, bone marrow, cerebrospinal fluid (CSF), saliva, mucous, sputum, semen, sweat, urine, or any other bodily fluids. “Blood sample” can refer to whole blood or any fraction thereof, including blood cells, red blood cells, white blood cells or leucocytes, platelets, serum and plasma. Samples can be obtained from a subject by means including but not limited to venipuncture, excretion, ejaculation, massage, biopsy, needle aspirate, lavage, scraping, surgical incision, or intervention or other means known in the art. In one embodiment the sample is a whole blood sample. A sample can include protein extracted from blood of a subject.

“Marker,” “markers,” biomarker,” or, “biomarkers,” all refer to a sequence characteristic of a particular variant allele (i.e., polymorphic site) or wild-type allele. A marker can include any allele, including wild-types alleles, SNPs, microsatellites, insertions, deletions, duplications, and translocations. A marker can also include a peptide encoded by an allele comprising nucleic acids. A marker in the context of the present teachings encompasses, without limitation, cytokines, chemokines, growth factors, proteins, peptides, nucleic acids, oligonucleotides, and metabolites, together with their related metabolites, mutations, variants, polymorphisms, modifications, fragments, subunits, degradation products, elements, and other analytes or sample-derived measures. Markers can also include mutated proteins, mutated nucleic acids, variations in copy numbers and/or transcript variants. Markers also encompass non-blood borne factors and non-analyte physiological markers of health status, and/or other factors or markers not measured from samples (e.g., biological samples such as bodily fluids), such as clinical parameters and traditional factors for clinical assessments. Markers can also include any indices that are calculated and/or created mathematically. Markers can also include combinations of any one or more of the foregoing measurements, including temporal trends and differences. As used herein, markers typically refer to sequence characteristics of the D-loop mtDNA, e.g., Tm and/or single or multiple SNPS and/or number of polymorphisms.

To “analyze” includes measurement and/or detection of data associated with a marker (such as, e.g., presence or absence of a SNP, allele, melting temperature (Tm) or constituent expression levels) in the sample (or, e.g., by obtaining a dataset reporting such measurements, as described below). In some aspects, an analysis can include comparing the measurement and/or detection against a measurement and/or detection in a sample or set of samples from the same subject or other control subject(s). The markers of the present teachings can be analyzed by any of various conventional methods known in the art.

A “dataset” is a set of data (e.g., numerical values) resulting from evaluation of a sample (or population of samples) under a desired condition. The values of the dataset can be obtained, for example, by experimentally obtaining measures from a sample and constructing a dataset from these measurements; or alternatively, by obtaining a dataset from a service provider such as a laboratory, or from a database or a server on which the dataset has been stored. Similarly, the term “obtaining a dataset associated with a sample” encompasses obtaining a set of data determined from at least one sample. Obtaining a dataset encompasses obtaining a sample, and processing the sample to experimentally determine the data, e.g., via measuring, sequencing, PCR, RT-PCR, microarray, contacting with one or more primers, contacting with one or more probes, antibody binding, or ELISA. The phrase also encompasses receiving a set of data, e.g., from a third party that has processed the sample to experimentally determine the dataset. Additionally, the phrase encompasses mining data from at least one database or at least one publication or a combination of databases and publications.

“Measuring” or “measurement” in the context of the present teachings refers to determining the presence, absence, quantity, amount, or effective amount of a substance in a clinical or subject-derived sample, including the presence, absence, or concentration levels of such substances, and/or evaluating the values or categorization of a subject's clinical parameters based on a control.

The term “acute coronary syndrome” encompasses all forms of unstable coronary artery disease.

The term “coronary artery disease” or “CAD” encompasses all forms of atherosclerotic disease affecting the coronary arteries. In particular, CAD includes obstructive CAD.

The term “FDR” means to false discovery rate. FDR can be estimated by analyzing randomly-permuted datasets and tabulating the average number of genes at a given p-value threshold.

The terms “highly correlated gene expression” or “highly correlated marker expression” refer to gene or marker expression values that have a sufficient degree of correlation to allow their interchangeable use in a predictive model of coronary artery disease. For example, if gene x having expression value X is used to construct a predictive model, highly correlated gene y having expression value Y can be substituted into the predictive model in a straightforward way readily apparent to those having ordinary skill in the art and the benefit of the instant disclosure. Assuming an approximately linear relationship between the expression values of genes x and y such that Y=a+bX, then X can be substituted into the predictive model with (Y−a)/b. For non-linear correlations, similar mathematical transformations can be used that effectively convert the expression value of gene y into the corresponding expression value for gene x. The terms “highly correlated marker” or “highly correlated substitute marker” refer to markers that can be substituted into and/or added to a predictive model based on, e.g., the above criteria. A highly correlated marker can be used in at least two ways: (1) by substitution of the highly correlated marker(s) for the original marker(s) and generation of a new model for predicting CAD risk; or (2) by substitution of the highly correlated marker(s) for the original marker(s) in the existing model for predicting CAD risk.

The term “myocardial infarction” refers to an ischemic myocardial necrosis. This is usually the result of abrupt reduction in coronary blood flow to a segment of the myocardium, the muscular tissue of the heart. Myocardial infarction can be classified into ST-elevation and non-ST elevation MI (also referred to as unstable angina). Myocardial necrosis results in either classification. Myocardial infarction, of either ST-elevation or non-ST elevation classification, is an unstable form of atherosclerotic cardiovascular disease.

The term “obtaining a dataset associated with a sample” encompasses obtaining a set of data determined from at least one sample. Obtaining a dataset encompasses obtaining a sample, and processing the sample to experimentally determine the data. The phrase also encompasses receiving a set of data, e.g., from a third party that has processed the sample to experimentally determine the dataset. Additionally, the phrase encompasses mining data from at least one database or at least one publication or a combination of databases and publications. A dataset can be obtained by one of skill in the art via a variety of known ways including stored on a storage memory.

As used herein “Corus” or “CorusCAD” refers to a commercially available test offered by CardioDx. This test is described in U.S. Pat. Nos. 9,122,777 and 8,914,240, each of which is herein incorporated by reference, in its entirety, for all purposes. In summary, Corus is a test where RNA is extracted from a sample of peripheral blood cells of a subject, converted to cDNA, and then assessed for the expression level of 23 distinct genes using RT-qPCR, followed by the transformation of the expression level data plus age and gender functions by an algorithm into a score that is predictive of the likelihood of CAD in the subject. Genes included in the Corus test are: S100A12, CLEC4E, S100A8, CASP5, IL18RAP, TNFAIP6, AQP9, NCF4, CD3D, TMC8, CD79B, SPIB, HNRPF, TFCP2, RPL28, AF161365, AF289562, SLAMF7, KLRC4, IL8RB, TNFRSF10C, KCNE3, and TLR4. The algorithm for producing the score is as shown below:
Define Norm₁=RPL28
Define Norm₂=(0.5*HNRPF+0.5*TFCP2)
Define NKᵤₚ=(0.5*SLAMF7+0.5*KLRC4)
Define T_{cell}=(0.5*CD3D+0.5*TMC8)
Define W_{cell}=(⅔*CD79B+⅓*SPIB)
Define Neut=(0.5*AQP9+0.5*NCF4)
Define Nᵤₚ=(⅓*CASP5+⅓*IL18RAP+⅓*TNFAIP6)
Define N_{down}=(0.25*IL8RB+0.25*TNFRSF10C+0.25*TLR4+0.25*KCNE3)
Define SCA₁=(⅓*S100A12+⅓*CLEC4E+⅓*S100A8)
Define AF₂=AF289562
Define TSPAN=1 if (AF161365-Norm2>6.27 or AF161365=NoCall), 0 otherwise
Define SEX=1 for Males, 0 for Females
Define Intercept
   For Males, INTERCEPT=2.672+0.0449*Age
   For Females, INTERCEPT=1.821+0.123*(Age-60), if negative set to 0
Define Score=INTERCEPT−0.755*(Nᵤₚ−N_{down})−0.406*(NKᵤₚ−T_{cell})−0.308*SEX*(SCA₁−Norm₁)−0.137*(B_{cell}+T_{cell})−0.548*(1−SEX)*(SCA₁−Neut)−0.482*SEX*(TSPAN)−0.246*(AF₂−Norm₂)

Methods

Disclosed herein are various methods of determining CAD risk in a subject from a sample. Such methods can include obtaining a dataset associated with a sample from a subject comprising data representing protein expression levels for one or more markers; and combining the data in the dataset to produce a score that is indicative of CAD risk associated with the sample. Such methods can include obtaining a dataset associated with a sample from a subject comprising data representing one or more clinical factors and data representing protein expression levels for markers; and combining the data in the dataset to produce a score that is indicative of CAD risk associated with the sample. Such methods can be computer-implemented, performed as physical assays, or a combination thereof. Such methods can be useful in informing later actions to be taken by the subject on whom the method is performed or by a physician that is assisting the subject. For example, a score that suggests a subject is at increased risk of CAD can be used by a physician to inform an action that is likely to reduce that risk, such as administering aspirin. Other actions that can be taken can include treating the subject, advising lifestyle changes to the subject, performing a procedure on the subject, performing further diagnostics on the subject, assessing the subject's health further, optimizing medical therapy, investigating non-cardiac etiologies of symptoms, or performing angiography on the subject.

Markers

“Marker,” “markers,” biomarker,” or, “biomarkers,” all refer to a sequence characteristic of a particular variant allele (i.e., polymorphic site) or wild-type allele. A marker can include any allele, including wild-types alleles, SNPs, microsatellites, insertions, deletions, duplications, and translocations. A marker can also include a peptide encoded by an allele comprising nucleic acids. A marker in the context of the present teachings encompasses, without limitation, cytokines, chemokines, growth factors, proteins, peptides, nucleic acids, oligonucleotides, and metabolites, together with their related metabolites, mutations, variants, polymorphisms, modifications, fragments, subunits, degradation products, elements, and other analytes or sample-derived measures. Markers can also include mutated proteins, mutated nucleic acids, variations in copy numbers and/or transcript variants. Markers also encompass non-blood borne factors and non-analyte physiological markers of health status, and/or other factors or markers not measured from samples (e.g., biological samples such as bodily fluids), such as clinical parameters and traditional factors for clinical assessments. Markers can also include any indices that are calculated and/or created mathematically. Markers can also include combinations of any one or more of the foregoing measurements, including temporal trends and differences.

Various markers are shown in the tables. In some aspects, a marker can include at least one of Adiponectin, APOA1, NT-proBNP, PIGF, and S100A8-MPO.

A marker can include one or more of corin, APOB, HSP70, RBP4, SERPINA12, NTproBNP, PIGF, adiponectin, APOA1, S100A8, MPO, S100A12, or TNFAIP6. A marker can include one or more of: APOB, HSP70, SERPINA12, NTproBNP, PIGF, adiponectin, APOA1, S100A8, MPO, S100A12, or TNFAIP6. A marker can include 1, 2, 3, 4, 5, 6, 7, 8, 9, 10, 11, or 12 of: corin, APOB, HSP70, RBP4, SERPINA12, NTproBNP, PIGF, adiponectin, APOA1, S100A8, MPO, S100A12, or TNFAIP6.

The terms “polypeptide,” “peptide” and “protein” are used interchangeably herein to refer to a polymer of amino acid residues. That is, a description directed to a polypeptide applies equally to a description of a peptide and a description of a protein, and vice versa. The terms apply to naturally occurring amino acid polymers as well as amino acid polymers in which one or more amino acid residues is a non-naturally encoded amino acid. As used herein, the terms encompass amino acid chains of any length, including full length proteins, wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, praline, serine, threonine, tryptophan, tyrosine, and valine) and pyrrolysine and selenocysteine. Amino acid analogs refers to compounds that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid. Reference to an amino acid includes, for example, naturally occurring proteogenic L-amino acids; D-amino acids, chemically modified amino acids such as amino acid variants and derivatives; naturally occurring non-proteogenic amino acids such as β-alanine, ornithine, etc.; and chemically synthesized compounds having properties known in the art to be characteristic of amino acids. Examples of non-naturally occurring amino acids include, but are not limited to, α-methyl amino acids (e.g., α-methyl alanine), D-amino acids, histidine-like amino acids (e.g., 2-amino-histidine, β-hydroxy-histidine, homohistidine), amino acids having an extra methylene in the side chain (“homo” amino acids), and amino acids in which a carboxylic acid functional group in the side chain is replaced with a sulfonic acid group (e.g., cysteic acid). The incorporation of non-natural amino acids, including synthetic non-native amino acids, substituted amino acids, or one or more D-amino acids into the proteins of the present invention may be advantageous in a number of different ways. D-amino acid-containing peptides, etc., exhibit increased stability in vitro or in vivo compared to L-amino acid-containing counterparts. Thus, the construction of peptides, etc., incorporating D-amino acids can be particularly useful when greater intracellular stability is desired or required. More specifically, D-peptides, etc., are resistant to endogenous peptidases and proteases, thereby providing improved bioavailability of the molecule, and prolonged lifetimes in vivo when such properties are desirable. Additionally, D-peptides, etc., cannot be processed efficiently for major histocompatibility complex class II-restricted presentation to T helper cells, and are therefore, less likely to induce humoral immune responses in the whole organism.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A derivative, or a variant of a polypeptide is said to share “homology” or be “homologous” with the peptide if the amino acid sequences of the derivative or variant has at least 50% identity with a 100 amino acid sequence from the original peptide. In certain embodiments, the derivative or variant is at least 75% the same as that of either the peptide or a fragment of the peptide having the same number of amino acid residues as the derivative. In certain embodiments, the derivative or variant is at least 85% the same as that of either the peptide or a fragment of the peptide having the same number of amino acid residues as the derivative. In certain embodiments, the amino acid sequence of the derivative is at least 90% the same as the peptide or a fragment of the peptide having the same number of amino acid residues as the derivative. In some embodiments, the amino acid sequence of the derivative is at least 95% the same as the peptide or a fragment of the peptide having the same number of amino acid residues as the derivative. In certain embodiments, the derivative or variant is at least 99% the same as that of either the peptide or a fragment of the peptide having the same number of amino acid residues as the derivative.

The term “modified,” as used herein refers to any changes made to a given polypeptide, such as changes to the length of the polypeptide, the amino acid sequence, chemical structure, co-translational modification, or post-translational modification of a polypeptide. The form “(modified)” term means that the polypeptides being discussed are optionally modified, that is, the polypeptides under discussion can be modified or unmodified.

In some aspects, a marker comprises an amino acid sequence that is at least 80, 85, 90, 91, 92, 93, 94, 95, 96, 97, 98, 99, or 100% identical to a relevant amino acid sequence or fragment thereof set forth in the Table(s) or accession number(s) disclosed herein. In some aspects, a marker comprises an amino acid sequence encoded by a polynucleotide that is at least 80, 85, 90, 91, 92, 93, 94, 95, 96, 97, 98, 99, or 100% identical to a relevant nucleotide sequence or fragment thereof set forth in Table(s) or accession number(s) disclosed herein. Accession numbers of certain markers are shown in Table 9.1.

Predictive Models

As disclosed herein the invention includes a method of generating a prediction model for likelihood of CAD in subjects. Also disclosed herein are methods of using the predictive model to determine the likelihood of CAD in a subject.

A predictive model can include, for example, a partial least squares model, a logistic regression model, a linear regression model, a linear discriminant analysis model, a ridge regression model, and a tree-based recursive partitioning model. In some embodiments, a predictive model can also include Support Vector Machines, quadratic discriminant analysis, or a LASSO regression model. See Elements of Statistical Learning, Springer 2003, Hastie, Tibshirani, Friedman; which is herein incorporated by reference in its entirety for all purposes.

Predictive model performance can be characterized by an area under the curve (AUC). In some embodiments, predictive model performance is characterized by an AUC ranging from 0.68 to 0.70. In some embodiments, predictive model performance is characterized by an AUC ranging from 0.70 to 0.79. In some embodiments, predictive model performance is characterized by an AUC ranging from 0.80 to 0.89. In some embodiments, predictive model performance is characterized by an AUC ranging from 0.90 to 0.99. AUC can range from 0.52 to 0.81, 0.50 to 0.99, 0.55 to 0.65, 0.50 to 0.70, 0.70 to 0.79, 0.80 to 0.89, or 0.90 to 0.99. AUC can be at least 0.5, 0.52, 0.6, 0.7, 0.8, or 0.81.

AIC can be used to measure model performance. Normal AIC is a combination of the log likelihood, or deviance, of the model adjusted by the number of parameters in the model. AIC can also be expressed as a corrected AIC (AICc) which is further adjusted for the number of cases available in a dataset from which a given estimate is calculated from. For example, corrected AIC can be calculated by: AICc=AIC+{2p(p+1)/n−p−1}, where p is the number of parameters in the model and n is the number of cases used in model fitting. AIC can range from 485 to 601, e.g., at least 490, 500, 510, 520, 530, 540, 550, 560, 570, 580, 590, 600 or greater (inclusive).

Relative Risk

In one embodiment, significance associated with one or more markers is measured by a relative risk. In a further embodiment, the significance is measured by a percentage. In one embodiment, a significant decreased risk is measured as a relative risk of at least about 1.2, including but not limited to: 1.2, 1.3, 1.4, 1.5, 1.6, 1.7, 1.8 and 1.9. In a further embodiment, a relative risk of at least 1.2 is significant. In a further embodiment, a relative risk of at least about 1.5 is significant. In a further embodiment, a significant increase in risk is at least about 1.7 is significant. In a further embodiment, a significant increase in risk is at least about 20%, including but not limited to about 25%, 30%, 35%, 40%, 45%, 50%, 55%, 60%, 65%, 70%, 75%, 80%, 85%, 90%, 95% and 98%. In a further embodiment, a significant increase in risk is at least about 50%.

Risk of CAD can be calculated by combining data representing expression levels of multiple protein markers, such as 2, 3, 4, 5, 6, 7, 8, 9, 10, 11, 12 or more protein markers. Risk of CAD can be calculated by combining data representing expression levels of multiple protein markers, such as 2, 3, 4, 5, 6, 7, 8, 9, 10, 11, 12 or more protein markers with data representing one or more clinical factors (e.g., age and/or gender). Such data combination will typically result in a score. Oftentimes such a score will be indicative of CAD risk. For example, a higher score for a given subject relative to a control subject having less than 50% stenosis in all major vessels as measured using Quantitative Coronary Angiography (QCA) can indicate an increased likelihood that the subject has CAD. Alternatively or in addition to, a lower score for a given subject relative to a control subject having greater than or equal to 50% stenosis in at least one major coronary vessel as measured using QCA can indicate a decreased likelihood that the subject has CAD.

A score produced via a combination of data can be useful in classifying, sorting, or rating a sample from which the score was generated. For example, a score can be used to classify a sample. A score can also be used to rate CAD risk for a given sample.

Assays

Examples of assays for one or more markers include DNA assays, microarrays, polymerase chain reaction (PCR), RT-PCR, Southern blots, Northern blots, antibody-binding assays, enzyme-linked immunosorbent assays (ELISAs), flow cytometry, protein assays, Western blots, nephelometry, turbidimetry, chromatography, mass spectrometry, immunoassays, including, by way of example, but not limitation, RIA, immunofluorescence, immunochemiluminescence, immunoelectrochemiluminescence, or competitive immunoassays, immunoprecipitation, and the assays described in the Examples section below. The information from the assay can be quantitative and sent to a computer system of the invention. The information can also be qualitative, such as observing patterns or fluorescence, which can be translated into a quantitative measure by a user or automatically by a reader or computer system. In an embodiment, the subject can also provide information other than assay information to a computer system, such as race, height, weight, age, gender, eye color, hair color, family medical history and any other information that may be useful to a user, such as a clinical factor described above.

Protein detection assays are assays used to detect the expression level of a given protein from a sample. Protein detection assays are generally known in the art and can include an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, amd immunoprecipitation. Reagents for use in such assays such as ELISA are shown in Table 9.2.

Protein based analysis, using an antibody as described above that specifically binds to a polypeptide encoded by an altered nucleic acid or an antibody that specifically binds to a polypeptide encoded by a non-altered nucleic acid, or an antibody that specifically binds to a particular splicing variant encoded by a nucleic acid, can be used to identify the presence in a test sample of a particular splicing variant or of a polypeptide encoded by a polymorphic or altered nucleic acid, or the absence in a test sample of a particular splicing variant or of a polypeptide encoded by a non-polymorphic or non-altered nucleic acid. The presence of a polypeptide encoded by a polymorphic or altered nucleic acid, or the absence of a polypeptide encoded by a non-polymorphic or non-altered nucleic acid, is diagnostic for a susceptibility to coronary artery disease.

In one aspect, the level or amount of polypeptide encoded by a nucleic acid in a test sample is compared with the level or amount of the polypeptide encoded by the nucleic acid in a control sample. A level or amount of the polypeptide in the test sample that is higher or lower than the level or amount of the polypeptide in the control sample, such that the difference is statistically significant, is indicative of an alteration in the expression of the polypeptide encoded by the nucleic acid, and is diagnostic. Alternatively, the composition of the polypeptide encoded by a nucleic acid in a test sample is compared with the composition of the polypeptide encoded by the nucleic acid in a control sample (e.g., the presence of different splicing variants). A difference in the composition of the polypeptide in the test sample, as compared with the composition of the polypeptide in the control sample, is diagnostic. In another aspect, both the level or amount and the composition of the polypeptide can be assessed in the test sample and in the control sample. A difference in the amount or level of the polypeptide in the test sample, compared to the control sample; a difference in composition in the test sample, compared to the control sample; or both a difference in the amount or level, and a difference in the composition, is indicative of a likelihood of CAD, either increased or decreased.

In addition, one of skill will also understand that the above described methods can also generally be used to detect markers that do not include a polymorphism.

Clinical Factors

In some embodiments, one or more clinical factors in an subject, e.g., a heart failure patient, can be assessed. In some embodiments, assessment of one or more clinical factors in a subject can be combined with a marker analysis in the subject to identify likelihood of CAD in the subject.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates.

A clinical factor can include age of a subject. A clinical factor can include gender of a subject. A clinical factor can include age and gender of a subject.

Various clinical factors are generally known to one of ordinary skill in the art to be associated with sudden cardiac events. In some embodiments, clinical factors known to one of ordinary skill in the art to be associated with coronary artery disease, such as an arrhythmia, can include age, gender, race, implant indication, prior pacing status, ICD presence, cardiac resynchronization therapy defibrillator (CRT-D) presence, total number of devices, device type, defibrillation thresholds performed, number of programming zones, heart failure (HF) etiology, HF onset, left ventricular ejection fraction (LVEF) at implant, New York Heart Association (NYHA) class, months from most recent myocardial infarction (MI) at implant, prior arrhythmia event in setting of MI or arthroscopic chondral osseous autograft transplantation (Cor procedure), diabetes status, Blood Urea Nitrogen (BUN), Cr, renal disease history, rhythm parameters to determine sinus v. non-sinus, heart rate, QRS duration prior to implant, left bundle branch block, systolic blood pressure, history of hypertension, smoking status, pulmonary disease, body mass index (BMI), family history of sudden cardiac death, B-type natriuretic peptide (BNP) levels, prior cardiac surgeries, medications, microvolt-level T-wave alternans (MTWA) result, and/or inducibility at electro-physiologic study (EPS).

In an embodiment, a condition can include one clinical factor or a plurality of clinical factors. In an embodiment, a clinical factor can be included within a dataset. A dataset can include one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, twenty or more, twenty-one or more, twenty-two or more, twenty-three or more, twenty-four or more, twenty-five or more, twenty-six or more, twenty-seven or more, twenty-eight or more, twenty-nine or more, or thirty or more overlapping or distinct clinical factor(s). A clinical factor can be, for example, the condition of a subject in the presence of a disease or in the absence of a disease. Alternatively, or in addition, a clinical factor can be the health status of a subject. Alternatively, or in addition, a clinical factor can be age, gender, chest pain type, neutrophil count, ethnicity, disease duration, diastolic blood pressure, systolic blood pressure, a family history parameter, a medical history parameter, a medical symptom parameter, height, weight, a body-mass index, resting heart rate, and smoker/non-smoker status. Clinical factors can include whether the subject has stable chest pain, whether the subject has typical angina, whether the subject has atypical angina, whether the subject has an anginal equivalent, whether the subject has been previously diagnosed with MI, whether the subject has had a revascularization procedure, whether the subject has diabetes, whether the subject has an inflammatory condition, whether the subject has an infectious condition, whether the subject is taking a steroid, whether the subject is taking an immunosuppressive agent, and/or whether the subject is taking a chemotherapeutic agent.

Computer Implementation

The methods of the invention, including the methods of generating a prediction model and the methods of for determining the likelihood of CAD in a subject, are, in some embodiments, performed on a computer.

In one embodiment, a computer comprises at least one processor coupled to a chipset. Also coupled to the chipset are a memory, a storage device, a keyboard, a graphics adapter, a pointing device, and a network adapter. A display is coupled to the graphics adapter. In one embodiment, the functionality of the chipset is provided by a memory controller hub and an I/O controller hub. In another embodiment, the memory is coupled directly to the processor instead of the chipset.

The storage device is any device capable of holding data, like a hard drive, compact disk read-only memory (CD-ROM), DVD, or a solid-state memory device. The memory holds instructions and data used by the processor. The pointing device may be a mouse, track ball, or other type of pointing device, and is used in combination with the keyboard to input data into the computer system. The graphics adapter displays images and other information on the display. The network adapter couples the computer system to a local or wide area network.

As is known in the art, a computer can have different and/or other components than those described previously. In addition, the computer can lack certain components. Moreover, the storage device can be local and/or remote from the computer (such as embodied within a storage area network (SAN)).

As is known in the art, the computer is adapted to execute computer program modules for providing functionality described herein. As used herein, the term “module” refers to computer program logic utilized to provide the specified functionality. Thus, a module can be implemented in hardware, firmware, and/or software. In one embodiment, program modules are stored on the storage device, loaded into the memory, and executed by the processor.

Embodiments of the entities described herein can include other and/or different modules than the ones described here. In addition, the functionality attributed to the modules can be performed by other or different modules in other embodiments. Moreover, this description occasionally omits the term “module” for purposes of clarity and convenience.

Methods of Therapy

The methods disclosed can be employed together with the treatment of subjects, e.g., through use of, e.g., diagnostic methods disclosed herein.

In some aspects, a subject has stable chest pain. In some aspects, a subject has typical angina or atypical angina or an anginal equivalent. In some aspects, a subject has no previous diagnosis of myocardial infarction (MI). In some aspects, a subject has not had a revascularization procedure. In some aspects, a subject does not have diabetes. In some aspects, a subject does not have a systemic autoimmune or infectious condition. In some aspects, a subject is not currently taking a steroid, an immunosuppressive agent, or a chemotherapeutic agent.

In some embodiments, methods can be employed for the treatment of other diseases or conditions associated with CAD. A therapeutic agent can be used both in methods of treatment of CAD, as well as in methods of treatment of other diseases or conditions associated with CAD.

The methods of treatment (prophylactic and/or therapeutic) can also utilize a therapeutic agent. The therapeutic agent(s) are administered in a therapeutically effective amount (i.e., an amount that is sufficient for “treatment,” as described above). The amount which will be therapeutically effective in the treatment of a particular individual's disorder or condition will depend on the symptoms and severity of the disease, and can be determined by standard clinical techniques. In addition, in vitro or in vivo assays may optionally be employed to help identify optimal dosage ranges. The precise dose to be employed in the formulation will also depend on the route of administration, and the seriousness of the disease or disorder, and should be decided according to the judgment of a practitioner and each patient's circumstances. Effective doses may be extrapolated from dose-response curves derived from in vitro or animal model test systems.

Therapies for a subject with CAD or a subject with an increased risk of CAD can include lifestyle changes, administration of therapeutics such as drugs, and undertaking one or more procedures. Lifestyle changes can include quitting smoking, avoiding secondhand smoke, eating a heart-healthy diet, regular exercise, achieving and/or maintaining a healthy weight, weight management, enrollment in a cardiac rehabilitation program, reducing blood pressure, reducing cholesterol, managing diabetes (if present), and keeping a healthy mental attitude. Therapeutics can include aspirin, antiplatelets, ACE inhibitors, beta-blockers, statins, PCSK9 targeting therapeutics (e.g., PCSK9 inhibitors such as monoclonal antibodies such as evolocumab, bococizumab, and alirocumab), and agina medicines such as nitroglycerin. Procedures include angioplasty (with or without stenting) and bypass surgery.

Kits

Also disclosed herein are kits for assessing CAD. Such kits can include reagents for detecting expression levels of one or markers and instructions for calculating a score based on the expression levels.

A kit can comprise a set of reagents for generating a dataset via at least one protein detection assay that is associated with a sample from the subject comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6; and instructions for generating a score indicative of CAD risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD. In certain aspects, the reagents can be selected from Table 9.2. In certain aspects, the reagents comprise one or more antibodies that bind to one or more of the markers, optionally wherein the antibodies are monoclonal antibodies or polyclonal antibodies. The reagents can include reagents for performing ELISA including buffers and detection agents.

A kit can further include software for performing instructions included with the kit, optionally wherein the software and instructions are provided together. For example, a kit can include software for generating a score indicative of CAD risk by mathematically combining data generated using the set of reagents.

A kit can include instructions for classifying a sample according to a score. A kit can include instructions for rating CAD risk using a score.

A kit can include instructions for obtaining data representing at least one clinical factor associated with a subject, wherein the at least one clinical factor comprises at least one of age and gender. In certain aspects, a kit can include instructions for mathematically combining the data representing at least one clinical factor with data representing protein expression levels to generate a score.

A kit can include instructions for use of a set of reagents. For example, a kit can include instructions for performing at least one protein detection assay such as an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, amd immunoprecipitation.

A kit can include instructions for taking at least one action based on a score for a subject, e.g., treating the subject, advising lifestyle changes to the subject, performing a procedure on the subject, performing further diagnostics on the subject, assessing the subject's health further, optimizing medical therapy, investigating non-cardiac etiologies of symptoms, or performing angiography on the subject.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

### Example 1: Identification and Testing of Protein Markers of CAD Risk

Study Population

Subjects enrolled in the multicenter PREDICT trial (ClinicalTrials.gov; NCT00500617, herein incorporated by reference) served as the starting population for this study. PREDICT enrolled subjects who were symptomatic or high risk asymptomatic patients referred for invasive coronary angiography with no known previous history of myocardial infarction or cardiac intervention.

For the purpose of these analyses two sets of PREDICT non-diabetic subjects were utilized, Set 1 for the initial assessment of candidate markers and Set 2 for validation of positive markers identified in Set 1 and subsequent multi-protein model development.
Set 1 included 187 subjects; 91 had obstructive CAD (>=50% stenosis in a major coronary artery by quantitative coronary angiography (QCA) or >=70% stenosis in a major coronary artery by clinical read.)
Set 2 included 199 subjects; 100 had obstructive CAD (>=50% stenosis in a major coronary artery by quantitative coronary angiography (QCA) or >=70% stenosis in a major coronary artery by clinical read.)

Basic clinical demographics for these 2 sets of subjects are shown in Table 1.1 and 2.1

TABLE 1.1
Set 1

| | Control | Case |
|---|---|---|
| N | 96 | 91 |
| Female | 38 (39.6%) | 39 (42.9%) |
| Age at Enrollment (yrs) | 62.63 +/− 11.72 | 64.32 +/− 11.10 |
| Race/Eth White not-hisp | 85 (88.5%) | 83 (91.2%) |
| Hypertension | 32 (33.7%) | 33 (36.7%) |
| Dyslipidemia | 70 (76.9%) | 66 (79.5%) |
| Diabetes | 0 (0.0%) | 0 (0.0%) |
| Smoker: Current | 18 (18.8%) | 21 (23.1%) |
| Smoker: Former | 46 (47.9%) | 36 (39.6%) |
| Smoker: Never | 32 (33.3%) | 34 (37.4%) |
| QCA Max Stenosis | 16.51 +/− 16.15 | 78.58 +/− 21.82 |

TABLE 2.1
Set 2

| | Control | Case |
|---|---|---|
| N | 99 | 100 |
| Female | 38 (38.4%) | 31 (31.0%) |
| Age at Enrollment (yrs) | 62.21 +/− 10.05 | 64.15 +/− 9.73 |
| Race/Eth White not-hisp | 87 (87.9%) | 88 (88.0%) |
| Hypertension | 37 (38.5%) | 34 (34.0%) |
| Dyslipidemia | 70 (74.5%) | 71 (75.5% |
| Diabetes | 0 (0.0%) | 0 (0.0%) |
| Smoker: Current | 15 (15.3%) | 28 (28.0%) |
| Smoker: Former | 41 (41.8%) | 46 (46.0%) |
| Smoker: Never | 42 (42.9%) | 26 (26.0%) |
| QCA Max Stenosis | 12.45 +/− 13.95 | 79.94 +/− 14.55 |

Methods

Study Logistics

The study was divided into 2 phases in regards to sets of potential biomarkers: Phase I evaluated 126 assays that had been previously characterized by MesoScale Discovery and were commercially available; Phase 2 evaluated 9 additional assays that were developed for CardioDx by MesoScale Discovery. Summaries of Phase 1 and 2 assays are provided below.

Phase I in Set 1
Set 1 included 187 subjects; 91 had obstructive CAD (>=50% stenosis in a major coronary artery by quantitative coronary angiography (QCA) or >=70% stenosis in a major coronary artery by clinical read.)

Panel Types
5 Validated Catalog (MesoScale standard configuration)
6 Custom Catalog (standard MesoScale assays in new multiplex)
4 Custom Prototype (new MesoScale assays and multiplex)
Each panel processed in five batches (“plates”)

Phase 2 in Set 1
9 assays were developed and run on 3 panels; for two protein targets (S100A12 and APOA1) two pairs of antibodies were tested. For Set 1 samples this was performed by MesoScale Discovery as a contract service using their electrochemiluminescence-based detection platform; for Set 2 samples this was performed at CardioDx using the MSD platform. Two metaproteins (S100A12/TNESRF10C and S100A8/MPO) were tested in which the values of well-correlated assays (r>0.7) were averaged. A third metaprotein (S10012/RAGE) was constructed based on biological interaction and assessed as a delta term.
   For APOA1, 2 pairs of antibodies were tested (Pair 2 and 4); ‘APOA1 Combined ‘presents the results using the average of the two.
   For S100A12, 2 pairs of antibodies were tested (Pair 1 and 2) at two plasma dilution levels (2-fold and 20-fold), ‘S100A12 Combined’ presents the average of the pairs using at a 2-fold plasma dilution.
   For the S100A12-RAGE term, the value is derived from the delta of the S100A12 Combined and RAGE results (S100A12-RAGE=S100A12 Combined minus RAGE)
   For the S100A12-TNFRSF10C term, the value is derived from the average of the S100A12 combined and TNFRSF10C results (S100A12-TNERSF10C=S10012/2 plus TNERSF10C/2).
   For the S100A8/MPO term, the value is derived from the average of the S100A8 and MPO values (S100A8-MPO=S100A8/2 plus MPO/2).

Reactions
Each well contained several (<10) assays
Duplicate reactions were run in adjacent wells
Each batch contains
   <=32 PREDICT patients, balanced between cases and controls.
   4 MSD control samples and 1-2 CardioDx control samples
   8 standards for signal calibration

Data Handling
Performed on both Phase 1 and 2 assays.
Used log-transformed estimates of concentration, except for Myoglobin and Osteonectin, which were near the ULOD, in which case the log 2-transformed signal was used.
Truncated low values to max of LLOD (lower limit of detection) and 2.5th percentile; high values to 97.5th percentile only.
All other adjustments performed subsequently

Single Analyte Model Fitting—Set 1
In Phase 1 the following screening and categories were applied to the 126 assays:
12 assays dropped due to low levels of detection (<30 observations above LLOD)
12 additional assays flagged as near LLOD (<85% above).
102 assays considered to be measured well, including a small number that are frequently above ULOD (upper limit of detection).

In Phas2 all assays measured protein levels at sufficient levels, well above the LLOD.
For both Phase 1 and 2 assays, three outcome variables were assessed:
   CAD: cases defined as subjects with >=70% lesion in a major coronary artery as determined by clinical read
   QCACAD: cases defined as subjects with >=50% lesion in a major coronary artery as determined by quantitative coronary angiography (QCA)
   QCAMaxStenosis: Maximum % lesion in a subject's coronary bed as determined by QCA used as a continuous variable.

The following models were fit for both Phase 1 and 2 assays:
QCAMaxStenosis as outcome, concentration as predictor of interest
   Fit directly by linear regression while including covariates, both clinical (age, sex, WBC) and processing (batch, column, row)
Binary CAD outcome (CAD or QCACAD50), concentration as predictor of interest
   Use adjusted concentration, fit univariate logistic regression.

Each assay was tested independently for association with disease. Where appropriate, concentrations were pre-adjusted for covariates.

Tables 3.1 and 4.1 summarize the results in regards to Phase 1 biomarkers showing significant association with CAD

TABLE 3.1
Summary of significant markers, by model. Good Signal/High confidence
signal = assays considered to be measured well, Near LLOD =
assays with <85% of sample above LLOD, lower confidence.

| | NumAssay | p < .01 | p < .05 | p < .1 |
|---|---|---|---|---|
| QCACAD50, Good | 103 | 3 (3%) | 9 (9%) | 16 (16%) |
| Signal | | | | |
| QCACAD50, Near | 12 | 0 (0%) | 1 (8%) | 1 (8%) |
| LLOD | | | | |
| CAD, Good Signal | 103 | 2 (2%) | 9 (9%) | 16 (16%) |
| CAD, Near LLOD | 12 | 0 (0%) | 2 (17%) | 3 (25%) |
| QCAMaxStenosis, | 103 | 1 (1%) | 8 (8%) | 17 (17%) |
| Good Signal | | | | |
| QCAMaxStenosis, | 12 | 0 (0%) | 0 (0%) | 2 (17%) |
| Near LLOD | | | | |

TABLE 4.1
Summary of directionality for assays with a high confidence signal.

| | NumAssay | p < .01 | p < .05 | p < .1 |
|---|---|---|---|---|
| QCACAD50, Upward | 103 | 3 (3%) | 0 (0%) | 15 (15%) |
| CAD, Upward | 103 | 2 (2%) | 8 (8%) | 12 (12%) |
| QCAMaxStenosis, Upward | 103 | 1 (1%) | 7 (7%) | 14 (14%) |
| QCACAD50, Downward | 103 | 0 (0%) | 0 (0%) | 1 (1%) |
| CAD, Downward | 103 | 0 (0%) | 1 (1%) | 4 (4%) |
| QCAMaxStenosis, | 103 | 0 (0%) | 1 (1%) | 3 (3%) |
| Downward | | | | |

TABLE 5.1
Individual p values and directionality for all CAD models using Phase 1 markers.

| | | | | | | QCA | |
|---|---|---|---|---|---|---|---|
| | | | QCA | QCA | QCA | Max | |
| | CAD | CAD | CAD50 | CAD50 | MaxStenosis | Stenosis | Detection |
| Protein | coef | pval | coef | pval | coef | pval | Level |
| A2M | −0.030 | 0.464 | −0.019 | 0.643 | 0.000 | 0.561 | Good |
| ActiveGLP1 | 0.024 | 0.866 | −0.083 | 0.560 | −0.002 | 0.423 | Good |
| Adiponectin | −0.240 | 0.030 | −0.214 | 0.055 | −0.003 | 0.038 | Good |
| Ang1 | −0.355 | 0.093 | −0.191 | 0.360 | −0.003 | 0.251 | Good |
| Ang2 | 0.105 | 0.263 | 0.088 | 0.338 | 0.001 | 0.713 | Good |
| B2M | 0.059 | 0.394 | 0.083 | 0.227 | 0.001 | 0.382 | Good |
| bFGF | 0.491 | 0.055 | 0.583 | 0.023 | 0.008 | 0.035 | Good |
| CA125 | 0.106 | 0.308 | 0.039 | 0.707 | 0.001 | 0.392 | Good |
| CA15.3 | 0.157 | 0.223 | 0.016 | 0.901 | 0.001 | 0.656 | Good |
| CA50 | −0.195 | 0.331 | −0.126 | 0.536 | −0.002 | 0.518 | Good |
| Calbindin | −0.020 | 0.791 | 0.009 | 0.907 | 0.000 | 0.970 | Good |
| CEA | −0.134 | 0.264 | −0.187 | 0.118 | −0.001 | 0.401 | Good |
| cKit | −0.022 | 0.637 | −0.044 | 0.340 | 0.000 | 0.649 | Good |
| Clusterin | −0.045 | 0.337 | −0.033 | 0.487 | 0.000 | 0.606 | Good |
| CPeptide | 0.066 | 0.483 | 0.023 | 0.808 | 0.001 | 0.463 | Good |
| CRP | 0.158 | 0.422 | 0.159 | 0.414 | 0.002 | 0.540 | Good |
| CTACK | −0.082 | 0.214 | −0.093 | 0.159 | −0.002 | 0.089 | Good |
| Cytokeratin8 | 0.231 | 0.110 | 0.249 | 0.087 | 0.003 | 0.086 | Good |
| ECadherin | 0.003 | 0.971 | 0.035 | 0.643 | 0.001 | 0.589 | Good |
| ENA78 | −0.151 | 0.507 | 0.116 | 0.606 | 0.002 | 0.514 | Good |
| Endoglin | −0.010 | 0.818 | −0.001 | 0.989 | 0.000 | 0.728 | Good |
| Eotaxin | −0.057 | 0.504 | 0.011 | 0.895 | 0.000 | 0.949 | Good |
| Eotaxin2 | −0.060 | 0.629 | −0.015 | 0.905 | 0.000 | 0.966 | Good |
| Eotaxin3 | 0.351 | 0.033 | 0.368 | 0.027 | 0.004 | 0.069 | Good |
| EPO | 0.026 | 0.816 | 0.025 | 0.818 | 0.001 | 0.760 | Good |
| ESelectin | −0.005 | 0.956 | 0.013 | 0.893 | 0.001 | 0.676 | Good |
| FABP3 | 0.058 | 0.351 | 0.043 | 0.491 | 0.000 | 0.814 | Good |
| FactorVII | −0.077 | 0.103 | 0.063 | 0.183 | −0.001 | 0.090 | Good |
| FasL | 0.130 | 0.120 | 0.059 | 0.467 | 0.001 | 0.527 | Good |
| FLT3Ligand | −0.032 | 0.622 | 0.030 | 0.634 | −0.001 | 0.565 | Good |
| Fractalkine | 0.024 | 0.574 | 0.038 | 0.386 | 0.000 | 0.593 | Good |
| GIP | −0.107 | 0.347 | 0.081 | 0.485 | −0.001 | 0.737 | Good |
| Glucagon | 0.019 | 0.699 | 0.013 | 0.794 | 0.000 | 0.786 | Good |
| I309 | 0.149 | 0.077 | 0.121 | 0.147 | 0.002 | 0.177 | Good |
| ICAM1 | 0.041 | 0.441 | 0.065 | 0.214 | 0.001 | 0.081 | Good |
| ICAM3 | 0.096 | 0.208 | 0.067 | 0.373 | 0.001 | 0.386 | Good |
| IFNG | 0.273 | 0.046 | 0.266 | 0.054 | 0.004 | 0.066 | Good |
| IL10 | −0.016 | 0.897 | 0.024 | 0.838 | 0.000 | 0.986 | Good |
| IL12orIL23p40 | 0.157 | 0.139 | 0.119 | 0.264 | 0.002 | 0.273 | Good |
| IL15 | 0.046 | 0.308 | 0.063 | 0.146 | 0.001 | 0.195 | Good |
| IL16 | 0.020 | 0.822 | 0.032 | 0.712 | −0.001 | 0.704 | Good |
| IL18 | 0.203 | 0.036 | 0.175 | 0.070 | 0.001 | 0.356 | Good |
| ILIA | −0.099 | 0.632 | 0.122 | 0.557 | −0.001 | 0.806 | Good |
| IL1Ra | 0.236 | 0.044 | 0.219 | 0.055 | 0.003 | 0.055 | Good |
| IL21 | −0.098 | 0.696 | 0.161 | 0.523 | −0.003 | 0.454 | Good |
| IL22 | 0.039 | 0.758 | 0.041 | 0.740 | 0.000 | 0.851 | Good |
| IL6R | −0.071 | 0.204 | 0.002 | 0.966 | −0.001 | 0.432 | Good |
| IL7 | −0.085 | 0.595 | 0.163 | 0.302 | 0.001 | 0.561 | Good |
| IL8 | 0.098 | 0.381 | 0.222 | 0.042 | 0.003 | 0.028 | Good |
| Insulin | 0.071 | 0.609 | 0.010 | 0.940 | 0.001 | 0.530 | Good |
| IP10 | 0.141 | 0.115 | 0.106 | 0.232 | 0.002 | 0.207 | Good |
| ITAC | −0.002 | 0.983 | 0.019 | 0.862 | 0.001 | 0.714 | Good |
| Leptin | 0.250 | 0.262 | 0.101 | 0.654 | 0.003 | 0.318 | Good |
| MCP1 | −0.076 | 0.259 | 0.008 | 0.903 | 0.001 | 0.572 | Good |
| MCP2 | 0.127 | 0.223 | 0.072 | 0.483 | 0.001 | 0.511 | Good |
| MCP4 | 0.110 | 0.283 | 0.080 | 0.432 | 0.002 | 0.226 | Good |
| MCSF | 0.112 | 0.251 | 0.127 | 0.191 | 0.001 | 0.298 | Good |
| MDC | −0.063 | 0.370 | 0.055 | 0.436 | −0.001 | 0.415 | Good |
| Mesothelin | −0.170 | 0.058 | 0.141 | 0.117 | −0.002 | 0.118 | Good |
| MIF | 0.288 | 0.112 | 0.417 | 0.022 | 0.005 | 0.045 | Good |
| MIG | 0.067 | 0.522 | 0.060 | 0.564 | 0.001 | 0.548 | Good |
| MIP1A | 0.270 | 0.021 | 0.228 | 0.053 | 0.003 | 0.058 | Good |
| MIP1B | 0.205 | 0.055 | 0.236 | 0.027 | 0.003 | 0.034 | Good |
| MIP3A | −0.084 | 0.565 | 0.057 | 0.686 | −0.001 | 0.529 | Good |
| MIP3B | 0.123 | 0.337 | 0.110 | 0.395 | 0.002 | 0.415 | Good |
| MIP5 | −0.017 | 0.772 | 0.003 | 0.954 | 0.000 | 0.746 | Good |
| MMP1 | −0.237 | 0.121 | 0.117 | 0.442 | −0.002 | 0.405 | Good |
| MMP3 | −0.042 | 0.629 | 0.026 | 0.753 | −0.001 | 0.599 | Good |
| MMP9 | 0.137 | 0.264 | 0.139 | 0.248 | 0.002 | 0.247 | Good |
| Myl3 | −0.013 | 0.928 | 0.011 | 0.936 | −0.001 | 0.460 | Good |
| Nectin4 | −0.036 | 0.527 | 0.017 | 0.768 | −0.001 | 0.534 | Good |
| NTproBNP | 0.523 | 0.022 | 0.582 | 0.009 | 0.007 | 0.016 | Good |
| OPGN | 0.056 | 0.322 | 0.074 | 0.180 | 0.001 | 0.155 | Good |
| Osteoactivin | 0.017 | 0.681 | 0.012 | 0.760 | 0.000 | 0.982 | Good |
| Osteocalcin | 0.002 | 0.981 | 0.066 | 0.478 | 0.001 | 0.710 | Good |
| Osteonectin | −0.124 | 0.080 | 0.062 | 0.379 | −0.001 | 0.242 | Good |
| Osteopontin | 0.137 | 0.129 | 0.106 | 0.232 | 0.001 | 0.277 | Good |
| PCadherin | −0.039 | 0.622 | 0.012 | 0.878 | −0.001 | 0.351 | Good |
| PlGF | 0.166 | 0.001 | 0.158 | 0.001 | 0.002 | 0.009 | Good |
| PSelectin | 0.097 | 0.224 | 0.076 | 0.337 | 0.001 | 0.504 | Good |
| RANTES | −0.264 | 0.160 | 0.018 | 0.924 | −0.001 | 0.778 | Good |
| Resistin | 0.011 | 0.865 | 0.102 | 0.104 | 0.001 | 0.290 | Good |
| SAA | 0.121 | 0.450 | 0.096 | 0.544 | 0.002 | 0.398 | Good |
| SCF | −0.003 | 0.966 | 0.002 | 0.973 | 0.000 | 0.698 | Good |
| sFas | −0.004 | 0.933 | 0.035 | 0.515 | 0.001 | 0.479 | Good |
| sFlt1 | −0.019 | 0.728 | 0.033 | 0.544 | 0.000 | 0.765 | Good |
| sMET | −0.025 | 0.429 | 0.038 | 0.222 | −0.001 | 0.151 | Good |
| TARC | −0.180 | 0.177 | 0.063 | 0.634 | −0.002 | 0.251 | Good |
| TenascinC | −0.036 | 0.429 | 0.001 | 0.981 | −0.001 | 0.406 | Good |
| Thrombomodulin | −0.019 | 0.752 | 0.015 | 0.807 | 0.000 | 0.677 | Good |
| Tie2 | 0.012 | 0.836 | 0.019 | 0.738 | 0.000 | 0.771 | Good |
| TNFA | 0.112 | 0.058 | 0.096 | 0.093 | 0.001 | 0.101 | Good |
| TNFB | 0.172 | 0.001 | 0.154 | 0.004 | 0.002 | 0.017 | Good |
| TNFRI | 0.008 | 0.909 | 0.039 | 0.554 | 0.000 | 0.713 | Good |
| TNFRII | 0.031 | 0.648 | 0.052 | 0.428 | 0.000 | 0.686 | Good |
| TPO | −0.018 | 0.702 | 0.015 | 0.753 | 0.000 | 0.761 | Good |
| TRAIL | −0.028 | 0.624 | 0.041 | 0.481 | −0.001 | 0.544 | Good |
| VCAM1 | 0.044 | 0.331 | 0.089 | 0.044 | 0.001 | 0.051 | Good |
| VEGFA | −0.061 | 0.573 | 0.017 | 0.879 | 0.000 | 0.818 | Good |
| VEGFC | −0.036 | 0.714 | 0.030 | 0.756 | 0.000 | 0.929 | Good |
| VEGFC.l | −0.139 | 0.172 | 0.023 | 0.819 | −0.001 | 0.626 | Good |
| VEGFD | 0.053 | 0.498 | 0.076 | 0.316 | 0.001 | 0.360 | Good |
| YKL40 | 0.101 | 0.496 | 0.212 | 0.150 | 0.003 | 0.131 | Good |
| GMCSF | −0.007 | 0.874 | 0.044 | 0.303 | 0.001 | 0.404 | Near |
| | | | | | | | LLOD |
| GROA | 0.013 | 0.915 | 0.057 | 0.651 | 0.001 | 0.459 | Near |
| | | | | | | | LLOD |
| IL17A | 0.005 | 0.968 | 0.032 | 0.773 | 0.001 | 0.671 | Near |
| | | | | | | | LLOD |
| IL17B | 0.137 | 0.247 | 0.159 | 0.185 | 0.002 | 0.195 | Near |
| | | | | | | | LLOD |
| IL17D | 0.137 | 0.078 | 0.111 | 0.160 | 0.002 | 0.063 | Near |
| | | | | | | | LLOD |
| IL2 | −0.005 | 0.930 | 0.002 | 0.965 | 0.001 | 0.502 | Near |
| | | | | | | | LLOD |
| IL33 | 0.064 | 0.723 | 0.027 | 0.882 | 0.000 | 0.986 | Near |
| | | | | | | | LLOD |
| IL5 | 0.265 | 0.014 | 0.214 | 0.047 | 0.003 | 0.054 | Near |
| | | | | | | | LLOD |
| IL6 | 0.065 | 0.124 | 0.060 | 0.127 | 0.001 | 0.102 | Near |
| | | | | | | | LLOD |
| IL8HA | 0.280 | 0.016 | 0.174 | 0.134 | 0.002 | 0.279 | Near |
| | | | | | | | LLOD |
| IL9 | 0.058 | 0.340 | 0.056 | 0.370 | 0.001 | 0.209 | Near |
| | | | | | | | LLOD |
| MCP3 | 0.108 | 0.159 | 0.061 | 0.422 | 0.002 | 0.125 | Near |
| | | | | | | | LLOD |
| CKMB | ND | ND | ND | ND | ND | ND | ND |
| cTnI | ND | ND | ND | ND | ND | ND | ND |
| cTnT | ND | ND | ND | ND | ND | ND | ND |
| IFNA | ND | ND | ND | ND | ND | ND | ND |
| IL12p70 | ND | ND | ND | ND | ND | ND | ND |
| IL13 | ND | ND | ND | ND | ND | ND | ND |
| IL1B | ND | ND | ND | ND | ND | ND | ND |
| IL25orIL17E | ND | ND | ND | ND | ND | ND | ND |
| IL4 | ND | ND | ND | ND | ND | ND | ND |
| Myoglobin | ND | ND | ND | ND | ND | ND | ND |
| PYY | ND | ND | ND | ND | ND | ND | ND |
| SDF1a | ND | ND | ND | ND | ND | ND | ND |

Significant p values are shown in bold.

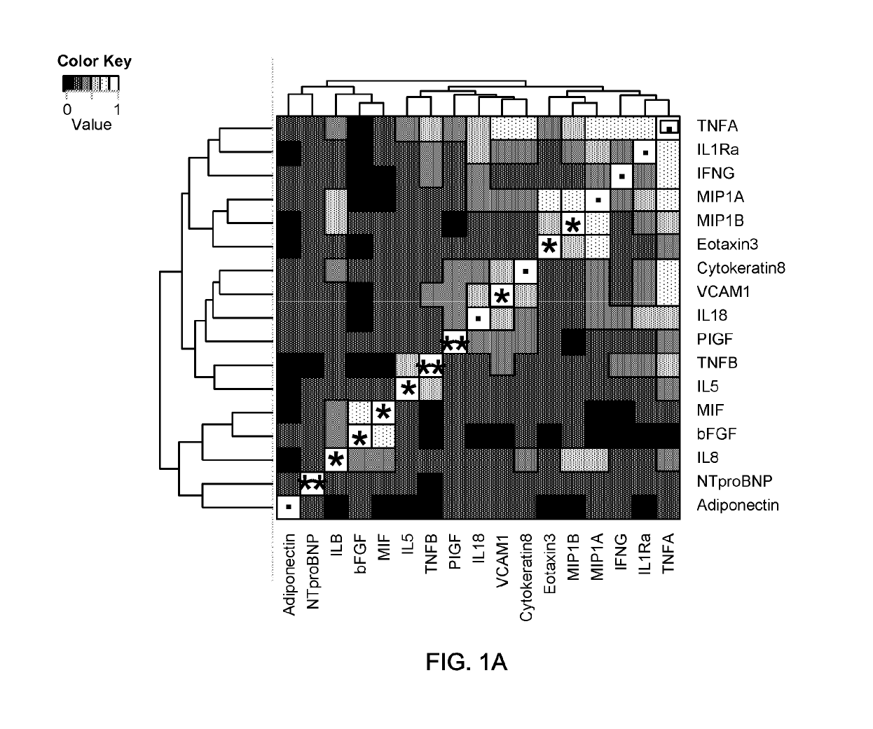

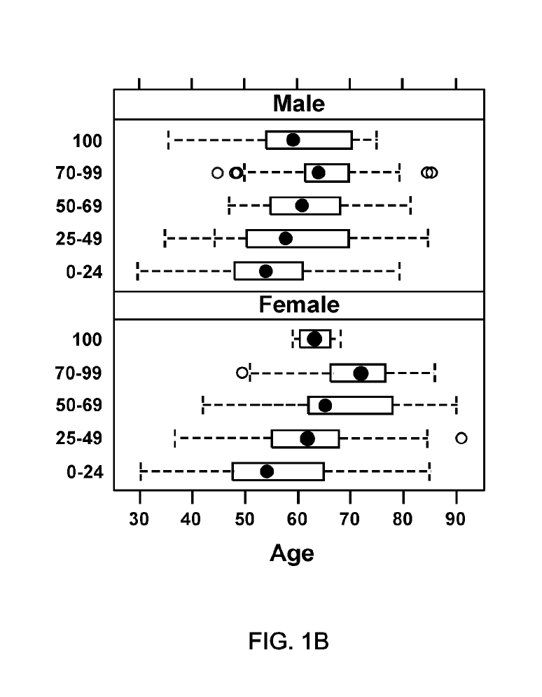

Correlation between top Phase 1 markers was assessed; overall, pairwise correlation was low (r<0.7) (FIG. 1).

Table 6.1a give individual p valves and directionality for all CAD models using Phase 2 markers in Set 1. Significant p values are in bold.

| | | | QCA | QCA | | QCA Max |
|---|---|---|---|---|---|---|
| | CAD | CAD | CAD50 | CAD50 | QCA Max | Stenosis |
| | Coef | pval | Coef | pval | StenosisCoef | pval |
| APOA1 Combined | −0.890 | 0.004 | −0.589 | 0.045 | −11.668 | 0.024 |
| APOA1 Pair2 | −0.853 | 0.004 | −0.599 | 0.039 | −11.563 | 0.024 |
| APOA1 Pair4 | −0.848 | 0.004 | −0.530 | 0.062 | −10.806 | 0.032 |
| MMP7 | 0.027 | 0.886 | 0.192 | 0.305 | 3.007 | 0.373 |
| MPO | 0.173 | 0.059 | 0.183 | 0.050 | 2.586 | 0.122 |
| PECAM1 | 0.072 | 0.794 | 0.399 | 0.157 | 3.980 | 0.433 |
| RAGE | −0.241 | 0.220 | −0.156 | 0.426 | −5.199 | 0.144 |
| S100A12 | 0.063 | 0.432 | 0.107 | 0.199 | 1.221 | 0.420 |
| S100A12 Pair1 20-fold | −0.069 | 0.624 | 0.078 | 0.595 | 1.166 | 0.663 |
| S100A12 Pair1 2-fold | 0.002 | 0.976 | 0.051 | 0.464 | 0.316 | 0.805 |
| S100A12 Pair2 20-fold | −0.023 | 0.773 | −0.008 | 0.924 | 0.330 | 0.822 |
| S100A12 Pair2 2-fold | 0.137 | 0.112 | 0.163 | 0.072 | 2.244 | 0.161 |
| S100A12/RAGE | 0.118 | 0.164 | 0.144 | 0.098 | 2.275 | 0.142 |
| S100A12/TNFRSF10C | 0.060 | 0.522 | 0.116 | 0.234 | 1.370 | 0.438 |
| S100A8 | 0.121 | 0.156 | 0.183 | 0.037 | 2.087 | 0.181 |
| S100A8/MPO | 0.157 | 0.087 | 0.197 | 0.036 | 2.506 | 0.135 |
| TNFAIP6 | 0.043 | 0.684 | 0.107 | 0.323 | 1.354 | 0.493 |
| TNFRSF10C | −0.097 | 0.633 | −0.090 | 0.663 | −3.524 | 0.346 |

Significant p values are in bold.

Model Building and Performance Estimates in Set 2.

In order to utilize multiple proteins in predicting a patient's disease status, two versions of disease likelihood score were produced by fitting L1-penalized logistic regression models (the “LASSO” method). The outcome variable for these models was a patient's CAD status, as defined by >=50% max stenosis if QCA was available; or if not, by >=70% max stenosis by clinical angiography and/or >=50% stenosis in the left main vessel.

The first version of the risk score was fitted using all 14 selected markers (Table 6.1), and is as follows:

SCORE1=0.03165626-0.126123955*APOA1+0.115560254*NT-ProBNP

The second version of the risk score was produced by restricting attention to the markers that were included in Set 1A models (Table 6.1). Due to the more restrictive initial selection, the resulting model was more permissive about including proteins and is as follows:

RISKSCORE2=0.033643483+0.288633218*NT-proBNP−0.259370805*APOA1−0.09760706*Adiponectin+0.067488037*P1GF+0.106117284*S100A8-MPO

Table 6.1b and 7.1 summarize the markers and coefficients for the two models side by side, including the model weights

TABLE 6.1b
Protein marker inputs for model generation

| | | All | Restrictive |
|---|---|---|---|
| | | Protein | Protein |
| Gene | Accession Number | Model | Model |
| Symbol | (gene/protein) | Input | Input |
| ADIPOQ | NM_004797.3/NP_004788.1 | Adiponectin | Adiponectin |
| APOA1 | NM_000039.1/NP_000030.1 | APOA1 | APOA1 |
| NPPB | NM_002521.2/NP_002512.1 | NT-proBNP | NT-proBNP |
| PGF | NM_002632.5/NP_002623.2 | PlGF | PlGF |
| S100A8 | NM_002964.4/NP_0029 | S100A8- | S100A8- |
| MPO | NM_000250.1/ | MPO | MPO |
| | NP_000241.1 55.2 | | |
| S100A12 | NM_005621.1/NP_005612.1 | S100A12- | NA |
| TNFAIP6 | NM_007115.3/NP_009046.2 | TNFAIP6 | |
| FGF2 | NM_002006.4/NP_001997.5 | bFGF | NA |
| IL6 | NM_000600.3/NP_000591.1 | IL6 | NA |
| CXCL8 | NM_000584.3/NP_000575.1 | IL8 | NA |
| MIF | NM_002415.1/NP_002406.1 | MIF | NA |
| ICAM1 | NM_000201.2/NP_000192.2 | sICAM1 | NA |
| VCAM1 | NM_001078.3/NP_001069.1 | sVCAM1 | NA |
| TNF | NM_000594.3/NP_000585.2 | TNFA | NA |
| LTA | NM_001159740.2/ | TNFB | NA |
| | NP_001153212.1 | | |

TABLE 7.1

| var | All Protein Model | Restrictive Protein Model |
|---|---|---|
| (Intercept) | 0.03165626 | 0.033643483 |
| APOA1 | −0.126123955 | −0.259370805 |
| Adiponectin | 0 | −0.09760706 |
| PlGF | 0 | 0.067488037 |
| S100A8-MPO | 0 | 0.106117284 |
| NT-proBNP | 0.115560254 | 0.288633218 |
| S100A12- | 0 | NA |
| TNFAIP6 | | |
| bFGF | 0 | NA |
| IL6 | 0 | NA |
| IL8 | 0 | NA |
| MIF | 0 | NA |
| sICAM1 | 0 | NA |
| sVCAM1 | 0 | NA |
| TNFA | 0 | NA |
| TNFB | 0 | NA |

Model performance was estimated via 2500 iteration of cross validation on random holdout sets of 14 patients; Area-Under-the-Curve (AUC) estimates are given in Table 8.1.

TABLE 8.1

| Model | AUC |
|---|---|
| “All” Protein | 0.63 |
| “Restrictive” Protein | 0.64 |

### Example 2: Validation, Model Building, and Analysis

Overview

The purpose of this analysis was to determine the combined performance of certain markers and/or factors at predicting obstructive CAD (oCAD). This process utilized stages of model building and selection, as well as some variable selection in the form of clinical covariate inclusion. The main analyses presented here center on the CADP2 group of PREDICT patients, which were independent of the PREDICT CADP1 set used to select the proteomics markers initially. The marker set forming the basis of this analysis is a composite of clinical data, Corus test results, and several proteomics data sets that were generated in different stages. Of these, the new results presented here are the addition of the 5 selected markers from Custom Set 2 to the previously selected 10 protein markers from the Catalog 126 and Custom Set 1. There were a total of N=472 patients with full data on the most recent proteomics data set (Custom Set 2), and this forms the basis of the group that was analyzed. Their clinical characteristics are summarized in Tables 1 and 2.

Methods

Cohort and Marker Selection

Markers for this experiment were selected from several sets of candidate markers previously assayed on the CADP1 set of patients (1A, 1B or both). From the Catalog 126 and Custom Set 1 experiments, the markers NT-proBNP, P1GF, S100A8, MPO, APOA1, Adiponectin, S100A12, and TNFAIP6) were selected. From the Custom Set 2 experiment using CADP1A patients (n=183, m=15), 5 markers were selected to continue into this validation set: APOB, corin, HSP70, RBP4, and SERPINA12. CADP1A is a group of matched cases and controls for age, gender, and some covariates, selected for extreme case and control status. The data from this discovery set was produced by Mesoscale (MSD), while the validation data was produced in-house, using antibody coated plates created by MSD for the prior discovery study.

Markers and Reagents

The accession numbers for markers are shown in Table 9.1. Reagents used to detect each marker via ELISA are shown in Table 9.2.

TABLE 9.1

| Protein | Uniprot ID | Entrez Gene ID |
|---|---|---|
| Hsp70 | P0DMV8 | 3303 |
| SerpinA12 | Q8IW75 | 145264 |
| corin | Q9Y5Q5 | 10699 |
| RBP4 | P02753 | 5950 |
| APOB | Q7Z7Q0 | 338 |
| NT-proBNP | P16860 | 4879 |
| ApoA1 | P02647 | 335 |
| Adiponectin | Q15848 | 9370 |
| PlGF | P49763 | 5228 |
| S100A8 | P05109 | 6279 |
| S100A12 | P80511 | 6283 |
| TNFAIP6 | P98066 | 7130 |
| MPO | P05164 | 4353 |

TABLE 9.2

| Protein | Ab type | Vendor | Catalog # |
|---|---|---|---|
| Hsp70 | capture | Mesoscale Diagnostics | K151EVD |
| Hsp70 | detection | Mesoscale Diagnostics | K151EVD |
| SerpinA12 | capture | R&D Systems | AF4410 |
| SerpinA12 | detection | R&D Systems | AF4410 |
| corin | capture | R&D Systems | MAB2209 |
| corin | detection | R&D Systems | AF2209 |
| RBP4 | capture | Mesoscale Diagnostics | K151LXD |
| RBP4 | detection | Mesoscale Diagnostics | K151LXD |
| APOB | capture | Abnova | MAB4023 |
| APOB | detection | R&D Systems | MAB4124 |
| NT-proBNP | capture | Mesoscale Diagnostics | K151JKC |
| NT-proBNP | detection | Mesoscale Diagnostics | K151JKC |
| ApoA1 | capture | R&D Systems | AF3664 |
| ApoA1 | detection | Mesoscale Diagnostics | 20150715-AAA-12 |
| Adiponectin | capture | Mesoscale Diagnostics | K151BXC |
| Adiponectin | detection | Mesoscale Diagnostics | K151BXC |
| PlGF | capture | Mesoscale Diagnostics | K151MED |
| PlGF | detection | Mesoscale Diagnostics | K151MED |
| S100A8 | capture | R&D Systems | MAB4570 |
| S100A8 | detection | R&D Systems | AF4570 |
| S100A12 | capture | R&D Systems | MAB10522 |
| S100A12 | detection | R&D Systems | AF1052 |
| TNFAIP6 | capture | R&D Systems | MAB2104 |
| TNFAIP6 | detection | R&D Systems | AF2104 |
| MPO | capture | Mesoscale Diagnostics | K151EEC |
| MPO | detection | Mesoscale Diagnostics | K151EEC |

Response Variables

The response variable used is a combined reference (continuous variable=Stenosis.Combo, case/control=CAD or CAD.RespNum), which defines a case as QCAMaxStenosis (QCA)>50%, if available. If unavailable, a case is defined as MaxStenosis>70%, otherwise all remaining patients are controls. MaxStenosis is the clinical angiographic read, while QCAMaxStenosis is the quantitative clinical angiography read result. Quantitative Coronary Angiography (QCA) is described in Garrone P, Biondi-Zoccai G, Salvetti I, Sina N, Sheiban I, Stella PR, Agostoni P. Quantitative coronary angiography in the current era: principles and applications. J Intery Cardiol. 2009 December; 22(6):527-36. doi: 10.1111/j.1540-8183.2009.00491.x. Epub 2009 Jul. 13. Review. PubMed PMID: 19627430.

Clinical Covariates (Clinical Factors)

For clinical covariates, earlier work had indicated that age and gender are important predictors of oCAD, so they were included in all main models. Some of the exploratory models do not include these predictors. Earlier work indicated some non-linearity in the relation-ship between oCAD, age and gender. To explore this, various splines for these predictors were put into different models. The main spline used was to include 3 knots for age, at 20, 60, and 80 years, based on the previous results.

Three other clinical covariates had been found in prior work to be important predictors within subsets of age and gender: Smoking status, Dyslipidemia diagnosis, and type of Chest Pain. These were included in some of the main models, encoded as binary variables based on prior observations. They were encoded as follows:
Smoking={1, Patient is female, <65 years old and a current smoker 0, o.w.
Dyslipidemia={1, Patient is female, <65 years old and diagnosed with Dyslipidemia 0, o.w.
Chest pain={1, Patient is over 65 years and has typical chest pain symptoms 0, o.w.

Signal Pre-Processing: Compilation, Inference, Truncation, and Transformation

The two marker data sets (Catalog 126+Custom Set 1, Custom Set 2) have different pre-processing steps to arrive at the actual values used in this analysis.

The Custom Set 2 data was generated by splitting the patients into 6 patient sets. For each protein to be assayed, duplicate plates were produced for each patient set. For the APOB assay, 3 of the patient sets were diluted to one level, while the second third of the patient sets were diluted to another, less-dilute level. There were noticeable and consistent shifts in the APOB values from the first 3 sets, relative to the second, even after the standard curve adjustment. Some of the other markers also showed evidence of systematic plate effects, although not as dramatic as the APOB shift. Additional normalization beyond the standard curve application was therefore performed by first log₂ transforming the concentration values, then subtracting off the deviations of individual plate medians from the overall median of each assay (centering the concentration values within each assay). Missing values were then imputed, and the mean of the two replicate values per sample, per assay was calculated. This was the original value used for analysis. No truncation or attempt to identify outliers was performed at this time after visual examination of the data implied that this was not warranted. To be more specific, the imputation was performed as follows: for samples with a missing value and an indication that the replicate was below the lower limit of detection, the imputed value was sampled from the range of (min. observed concentration, 2.5 percentile) with uniform probability. For samples with a missing value and an indication that they were above the upper limit of quantification, the value that was imputed was sampled with uniform probability from the range (97.5 percentile max. observed concentration). For samples that had missing values, but no indication that they were above or below limits of detection, if the replicate value was non-missing, this was used to replace the missing replicate value. There were no cases where both replicates of the sample for a marker were missing and no below or above limits of quantification flag was given. Imputed values were then truncated to ±3 MAD (median absolute deviation) of the study median, as calculated within each marker.

Missingness

There was a small amount of missing data in the data set which was complete for all Custom Set 2 markers (N=472). In general, the strategy that was taken was to impute values for the missing data because of its low frequency. The exception was that two of these subjects were missing data for all 10 Catalog 126/Custom Set 1 markers, and these were excluded from further analysis. The imputation details are as follows: For the Catalog 126 markers, three subjects were missing S100A8, S100A12, MPO and TNFAIP6 data. These subjects were imputed to have the median value for each marker in the data set.

Thirteen subjects were missing Corus scores. These were imputed to be the median Corus score by age group and gender of the subject, where age group was defined here as 25-40, 41-50, 51-60, 61-70 and 71-95 years of age. This bucketing was selected because of the importance of the 60 year old cutoff in the Corus female scores, and to create reasonably similar sized groups.

For the clinical covariates, 24 subjects were missing a Dyslipidemia diagnosis, and two additional subjects were missing smoking and chest pain data. See details in the model selection section, but due to the gender-specific coding of these covariates in the models, if the subject was of a gender or gender*age group that was automatically treated as 0, this was the imputed value for that subject and covariate. If the subject was in the gender*age group that had the potential to have a 1 value, the imputed value was sampled from a bivariate variable with probability of being 1 the frequency of that category in the patient group as a whole. For example, for Dyslipidemia, the 16 males subjects with missing data were imputed to have a 0 value for this, the seven females who were younger than 65 years were imputed to be 1 with a probability of 0.38, which is the frequency of Dyslipidemia in the entire patient set, and the remaining female was imputed to be 0.

Five subjects were missing Diamond-Forrester predicted values. Two of these were the same subjects missing the Chest Pain variables above. These were imputed to have intermediate risks for their age group and sex. The other three were imputed to have the risks associated with their age group, sex, and chest pain symptoms.

Data Characterization

TABLE 1
Summary of categorical clinical covariates
by case and control group

| | | Ct. | Pct. | Ct. | Pct. |
|---|---|---|---|---|---|
| Variable | Category | Ctrl | Ctrl | Case | Case |
| Gender | Female | 186 | (59%) | 44 | (28%) |
| | Male | 130 | (41%) | 112 | (72%) |
| RaceGrp | White | 267 | (84%) | 138 | (88%) |
| | Non-Hisp. | | | | |
| | Other | 49 | (16%) | 18 | (12%) |
| Smoke Group | Current | 62 | (20%) | 37 | (24%) |
| | Former | 114 | (36%) | 72 | (46%) |
| | Never | 138 | (44%) | 47 | (30%) |
| | Missing | 2 | (1%) | 0 | (0%) |
| HTNGroup | No HTN | 109 | (34%) | 45 | (29%) |
| | Con- | 195 | (62%) | 110 | (71%) |
| | trolledHTN | | | | |
| | HTN | 8 | (3%) | 1 | (1%) |
| | Missing | 4 | (1%) | 0 | (0%) |
| Dyslip | No Dyslip | 110 | (35%) | 40 | (26%) |
| | Dyslip | 190 | (60%) | 108 | (69%) |
| | Missing | 16 | (5%) | 8 | (5%) |
| ChestPainGroup | None | 85 | (27%) | 44 | (28%) |
| | Atypical | 108 | (34%) | 42 | (27%) |
| | angina | | | | |
| | Typical | 121 | (38%) | 70 | (45%) |
| | angina | | | | |

FIG. 1B shows distribution of percent stenosis across genders and age groups.

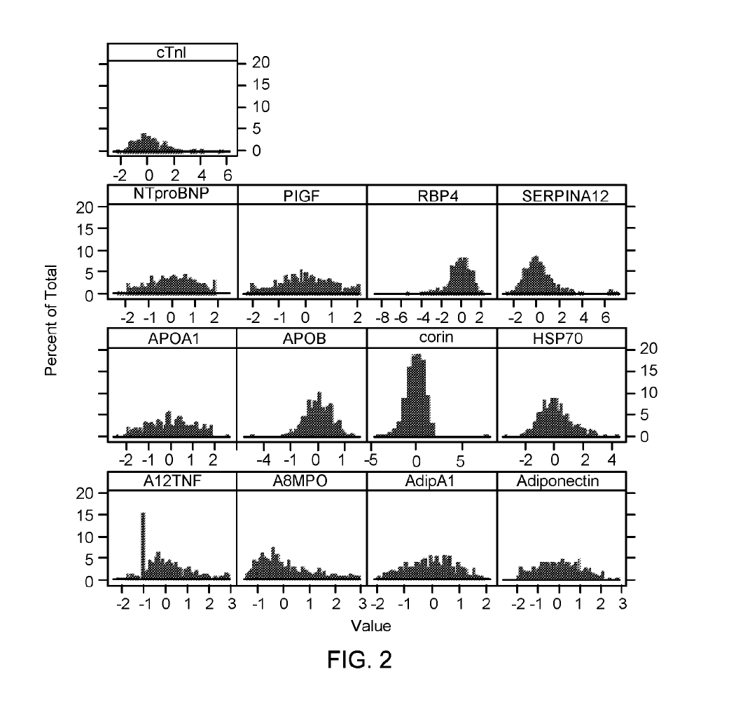

FIG. 2 shows marginal distributions of protein markers (log transformed, centered and scaled values).

TABLE 2
Summary statistics for continuous clinical covariates by case and control groups.

| Variable | NA.Ctrl | NA.Case | Ctrl.Mean | Ctrl.StdDev | Case.Mean | Case.StdDev |
|---|---|---|---|---|---|---|
| Age | 0 | 0 | 57.19 | 11.23 | 64.54 | 10.25 |
| Corus.Raw | 10 | 3 | −1.23 | 1.13 | −0.08 | 0.98 |
| Corus.Tfm | 10 | 3 | 15.81 | 9.89 | 25.9 | 8.32 |
| DF.p | 4 | 1 | 28.67 | 28.6 | 45.72 | 34.13 |
| Fram | 0 | 0 | 6.99 | 6.8 | 13.1 | 7.4 |
| Stenosis.Combo | 0 | 0 | 15.38 | 16.15 | 77.91 | 16.15 |
| WBC | 5 | 3 | 6.8 | 1.65 | 6.82 | 1.77 |

‘NA’ columns are counts of missing data for that variable of the original 472 patients.
‘DF.p’ is Diamond-Forrester probability, while ‘Fram’ is Framingham probability.

TABLE 3
Counts of subjects by percentage of stenosis for Young
and Older (>=65 years) subjects and gender.

| | |
|---|---|
| Females | Males |

| Pct. Stenosis | Young | Older | Young | Older |
|---|---|---|---|---|
| 0-24 | 96 | 31 | 70 | 14 |
| 25-49 | 33 | 23 | 28 | 15 |
| 50-69 | 9 | 10 | 22 | 9 |
| 70-99 | 5 | 19 | 34 | 24 |
| 100 | 3 | 1 | 16 | 10 |

Results

TABLE 4
Odds Ratios for individual marker models. All others
were run on the total CADP2 patient (N = 470).

| | | Low- | Up- | | |
|---|---|---|---|---|---|
| Model | OR | er | per | pVal | AIC |
| CAD ^{~} Gender + Age + APOA1 | 0.73 | 0.58 | 0.91 | 0.0056 | 504 |
| CAD ^{~} Gender + Age + | 0.76 | 0.60 | 0.95 | 0.0178 | 506 |
| Adiponectin | | | | | |
| CAD ^{~} Gender + Age + | 0.86 | 0.73 | 1.00 | 0.0493 | 508 |
| SERPINA12 | | | | | |
| CAD ^{~} Gender + Age + HSP70 | 0.88 | 0.71 | 1.09 | 0.2398 | 511 |
| CAD ^{~} Gender + Age + RBP4 | 0.92 | 0.78 | 1.10 | 0.3664 | 511 |
| CAD ^{~} Gender + Age + corin | 0.99 | 0.79 | 1.22 | 0.8946 | 512 |
| CAD ^{~} Gender + Age + PlGF | 1.16 | 0.94 | 1.44 | 0.1793 | 510 |
| CAD ^{~} Gender + Age + APOB | 1.17 | 0.95 | 1.46 | 0.1481 | 510 |
| CAD ^{~} Gender + Age + A8MPO | 1.18 | 0.96 | 1.46 | 0.1177 | 510 |
| CAD ^{~} Gender + Age + | 1.19 | 0.95 | 1.49 | 0.1368 | 510 |
| NTproBNP | | | | | |
| CAD ^{~} Gender + Age + A12TNF | 1.22 | 0.98 | 1.51 | 0.0728 | 509 |

Multivariate Model Building

For the model building, there were several things to consider, including marker selection, the amount of complexity to specify for each term, and the amount of summarization to use to account for collinearity of model terms. Several decisions on these items had been made in prior analyses, and these were carried forward. With regards to the marker selection, a set of candidate markers had been selected as top hits from three previous discovery data sets using the CADP1 patient groups (the Catalog 126, Custom 1 and Custom 2 sets). The use of clinical covariates in models was limited to top predictors identified in previous analyses.

With regards to collinearity and the optimal amount of summarization of the predictor variables, two pairs of markers had been previously identified as being highly correlated (S100A8, MPO) and (S100A12, TNFAIP6). The mean value of each of these pairs was used in all models, rather than the individual marker values due to the extent of the correlation, and these are referred to elsewhere herein as A8MPO and A12TNF. The data currently available for modelling the Catalog 126 and Custom Set 1 was pre-processed, including some form of outlier identification and removal, centering and scaling and estimation of a ‘Batch’ effect. Additionally, some other predictor variables showed some correlation amongst themselves. The largest of these was the pair of Adiponectin and APOA1. The mean of these two values in their centered and scaled forms was used and use of this as a single term in models due to current availability of the data, for those models that looked at the effect additional summarization would have on the fit.

Following Harrell's general rules of thumb, and based on an approximate N number of 440 subjects available, the target for the total number of degrees of freedom available for model selection was set at app. N/15=29. The set of predictor variables that were of interest were determined to have for the minimal full linear model: 7 parameters for the Catalog126+Custom Set 1 (6 markers after combining into A8MPO and A12TNF plus a Batch adjustment term), 5 parameters for the Custom Set 3, and 5 clinical covariates plus an overall intercept and a term for the Catalog model, or 19 total parameters. This left roughly 10 degrees of freedom available for modelling non-linear complexity that could reasonably be specified in the models. Complexity was partitioned out in the rank order of the predictor strengths, based on previous results. The first priority was to model complexity of the relationship of Age with oCAD. Then NTproBNP, HSP70, APOA1, RBP4, Adiponectin, and corin were the rank order of the previous effect estimate strengths. After some consideration, only Age and NTproBNP non-linearity were explored in these models, due to sample size. Without being bound by theory, it is thought that further model optimization could be pursued during algorithm development based on the results observed here.

Based on these calculations, a pre-specified set of 11 main models to address these primary questions of interest in modelling (summarization, complexity, and some limited marker selection), was compiled. To protect against inflation of model performance estimates, and yet still be able to use all the available data for model selection, Efron's optimism bootstrap was employed for all model performance measure estimates such as AUC, Sensitivity, etc. It was found that optimism estimates appeared to be converging after approximately 400 bootstrap iterations were performed. In the end 1000 iterations per main model were run, for these results.

Independence of Information

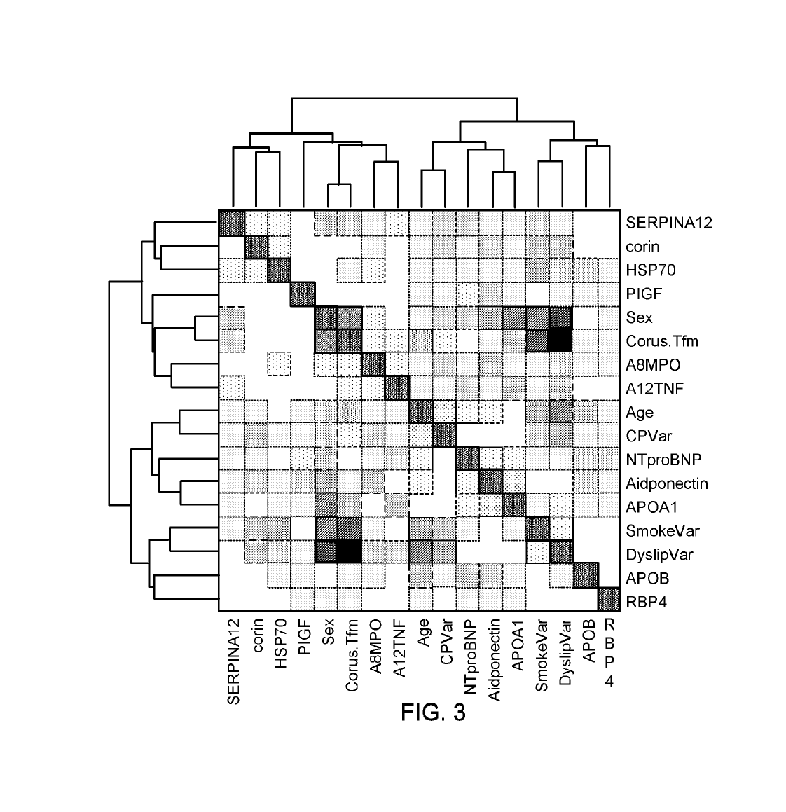

During the model planning process, the independence of the predictor variables were assessed, to determine if any correlated variables might be more optimally represented by summarization of their data into a single variable. Several measures of similarity were considered, including the rank correlation measure of Spearman (Table 18 and FIG. 3), this value squared, and Hoeffding's D statistic, which should re-elect monotonic similarities and non-linear similarities among predictors, respectively. FIG. 3 shows rank correlations among pairs of predictor variables. After examining the distributions of these measures in the predictor set under consideration, consideration, a cut point above which pairs looked unusually similar was made, and summarization of these pairs of markers was explored as a part of the model building process.

TABLE 5
Pairs with correlations above the cut
points of the similarity statistics.

| Var1 | Var2 | Spearman | SpearSq | HoeffdingD |
|---|---|---|---|---|
| A12TNF | A8MPO | 0.34 | 0.12 | 0.04 |
| Adiponectin | APOA1 | 0.48 | 0.23 | 0.08 |
| Adiponectin | NTproBNP | 0.33 | 0.11 | 0.03 |
| Age | Corus.Tfm | 0.53 | 0.28 | 0.09 |
| Age | CPVar | 0.46 | 0.21 | 0.02 |
| Age | NTproBNP | 0.32 | 0.10 | 0.03 |
| Corus.Tfm | DyslipVar | −0.55 | 0.31 | 0.04 |
| Corus.Tfm | Sex | 0.67 | 0.45 | 0.09 |
| DyslipVar | Sex | −0.49 | 0.24 | 0.01 |

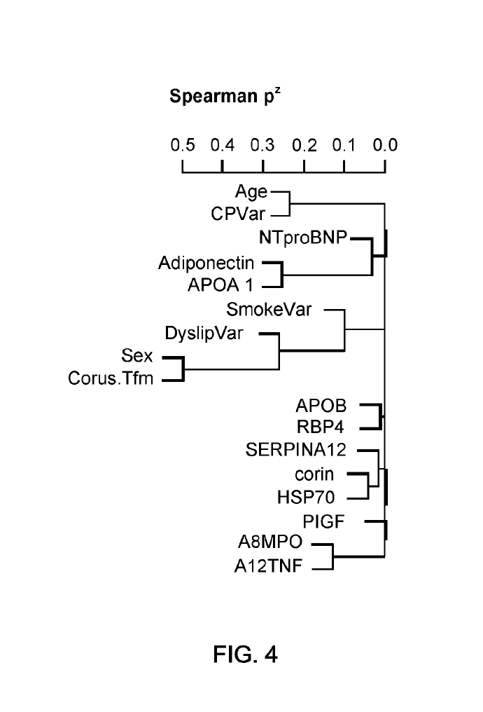

FIG. 4 shows a cluster diagram of the Spearman's non-parametric measure of correlation among the quantitative variables in the models. The measure is expressed as the square of this statistic to deal with negative correlation values. This measure should reflect monotonic, non-linear relationships.

Main Model Set

The main models considered were all logistic regression models, with the binary response of oCAD>=50% by QCA is a case, oCAD>=70% is a case if QCA was unavailable, all others were controls (Table 7). Because of the form of the available Catalog126+Custom Set 1 data which had a Batch intercept effect that the terms in these sets needed to be adjusted for, it was decided to create hierarchical models, where first a model was fitted for just this set of data alone. The predicted model values (on the Xβ scale) were calculated for each subject and used to create an additional variable, called “Catalog” models (Table 6). There were 5 such base-level Catalog models considered. This Catalog term was then put into the higher level main model as a single predictor. The models are described below. The general strategy for the main models was to explore the effects of increased predictor complexity, increased predictor summarization, and the effects of both together.

TABLE 6
The form of the various Catalog Models. The response is
a binary variable indicating Case or Control status.
Catalog Models

| | |
|---|---|
| Model A | CAD ~ −1 + Batch + Adiponectin + APOA1 + NTproBNP + |
| | PlGF + A8MPO + A12TNF |
| Model B | CAD ~ ModelA, except substitute ns(NTproBNP, 3) |
| Model C | CAD ~ ModelA, except substitute AdipA1 |
| Model D | CAD ~ ModelA, except substitute AdipA1 + |
| | ns(NTproBNP, 3) |
| Model E | CAD ~ ModelA + NTproBNP * Age |
| Model F | CAD ~ ModelA, except no APOA1 |
| Model G | CAD ~ −1 + ns(Age, knots = kn) * Gender |

‘ns(NTproBNP, 3)’ indicates a spline with 3 evenly spaced knots fitted to the NTproBNP marker values.
‘AdipA1’ indicates the mean of Adiponectin and APOA1.
A ‘*” indicates an interaction term. In this case, the individual terms plus an interaction term are actually fitted in the model.
A ‘−1’ indicates that Batch is to be used as an intercept term. Model G has a 3-knot spline fitted to each gender separately.

Model Performance

AIC Values and Determining the Best Model

The ability of each model to explain the variation in the data was compared using two statistics, AIC, which is the deviance of the model plus two times the number of parameters estimated by the model, and AIC_{C}, which is more severely penalized than the original AIC for the number of parameters in the model, relative to the number of subjects in the data set. AIC_{C} can be calculated as

$${AIC}_{c} = AIC + \frac{2 p ( p + 1 )}{n - p - 1} .$$

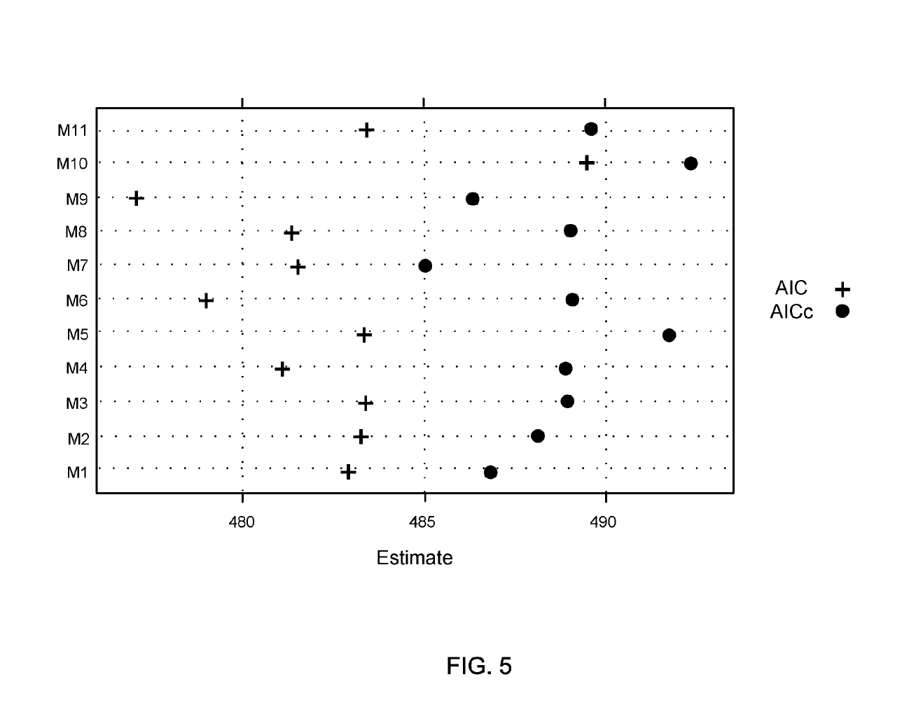

The median AIC values for the main models are shown in FIG. 5 (see also Appendix Table 19). FIG. 5 shows the median AIC and corrected AIC values for main models M1 through M11. Medians were calculated from the AICs obtained from all bootstrap iterations. Lower values indicate better fits of the model to the data.

With regards to AIC, models 6 and 9 look the most promising, but with the AIC_{c} measure, Model 7 is superior. Models 6 and 9 are similar to each other, both with non-linear age and gender splines, the summarization of Adiponectin and APOA1 into a single term, and a 3-knot spline fitted to NTproBNP. Model 9 additionally has the clinical covariates. Model 7 is a relatively simple linear, additive model, differing only from Model 1 in the combined Adiponectin—APOA1 term. Since Model 7 has adequate AIC, while Models 6 and 9 look less appealing by AIC_{C}, because of the high variability observed in the coefficients fitted to the spline terms among the bootstrap models, and due to the reduced complexity of Model 7, which could be of benefit in diagnostic development, Model 7 was selected as the model to use as a reference point for the performance of the current proteomic marker set after Discovery efforts.

TABLE 7
The form of the Main Models. The response is a binary
variable indicating Case or Control status.

Main
Models

| | | |
|---|---|---|
| Model 1 | Basic Linear Model - All | C AD ~ Gender + Age + |
| | Protein Markers | Catalog A + Set3 |
| Model 2 | Complexity - Age Interactions | C AD ~ Age * Gender + |
| | with Gender and NTproBNP | CatalogE + Set3 |
| Model 3 | Basic Linear Model + | C AD ~ Model1 + ChestPain + |
| | Clin. Co-var. | Smoking + Dyslipidemia |
| Model 4 | Complexity - gender specific, | C AD ~ ns(Age, knots = kn) * |
| | non-linear Age functions and | Gender + CatalogB + Set3 |
| | NTproBNP | |
| Model 5 | Complexity - non-linear Age | C AD ~ Gender + ns(Age, 4) + |
| | and NTproBNP | CatalogB + Set3, except |
| | | ns(HSP70, 3) |
| Model 6 | Complexity and Clin. Covar. | C AD ~ Model4 + ChestPain + |
| | | Smoking + Dyslipidemia |
| Set 3 | | =corin + APOB + HSP70 + |
| | | RPB4 + SERPINA12 |

‘ns(Age, knots = kn)’ indicates a spline with knots fitted to at ages 20, 60 and 80 years.
CatalogX indicates the Xβ values from the corresponding Catalog model.
A * indicates an interaction term. In this case, the individual terms plus an interaction term are actually fitted in the model.

TABLE 8
The form of the Main Models (cont.). The response is
a binary variable indicating Case or Control status.

Main
Models

| | | |
|---|---|---|
| Model 7 | Summarization - | C AD ~ Gender + Age + CatalogC + |
| | use AdipA1 | Set3 |
| Model 8 | Summarization and | C AD ~ CAD ~ ns(Age, knots = |
| | Complexity | kn) * Gender + CatalogD + Set3 |
| Model 9 | Summarization, | C AD ~ Model8 + ChestPain + |
| | Complexity | Smoking + Dyslipidemia |
| | and Clin. Covar | |
| Model 10 | Simplify Lab | C AD ~ Gender + Age + CatalogC + |
| | Processes | Set3, except no APOA or APOB |
| Model 11 | Use complex | C AD ~ CatalogG + CatalogC + Set3 |
| | Age*Gender | |
| | as single term | |
| Set 3 | | =corin + APOB + HSP70 + RPB4 + |
| | | SERPINA12 |

CatalogX indicates the Xβ values from the corresponding Catalog model.
A * indicates an interaction term. In this case, the individual terms plus an interaction term are actually fitted in the model.

Odds Ratios for the Selected Model

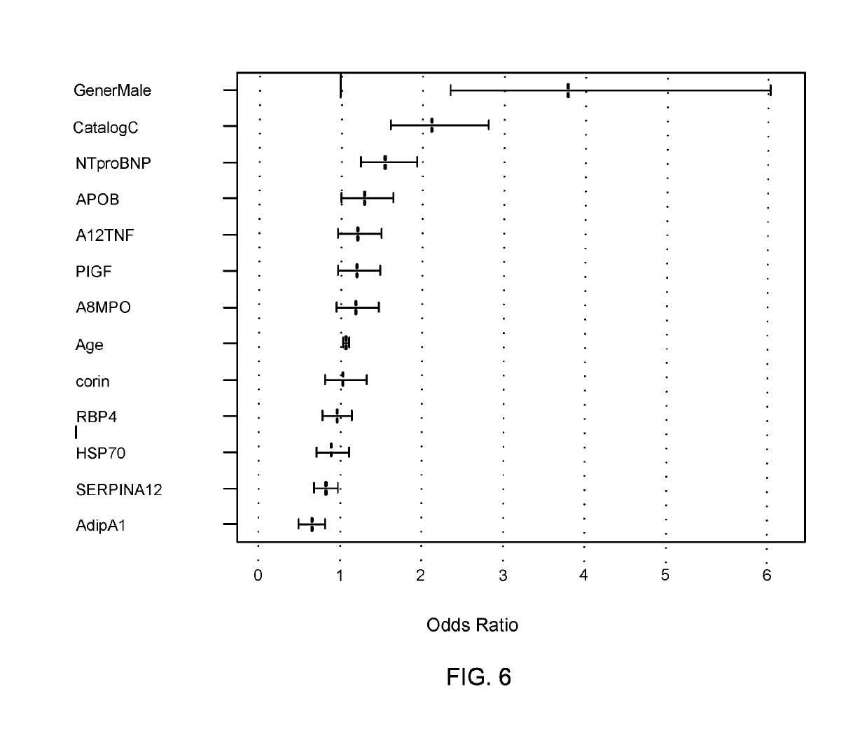

For explorative purposes, the odds ratios for the final Model 7, as fitted to the full CADP2 data set are shown in FIG. 6 and given in Table 20. There were some differences in direction and individual effect sizes between the prior results in a smaller discovery set and the current results (Table 9). FIG. 6 shows estimated odds ratios for all markers using Model 7. Note that the markers NTproBNP, A12TNF, P1GF, A8MPO and AdipA1 are fitted in the model term Catalog C, which is then fitted in the main model as a single term. Because of this, odds ratios are shown individually and collectively in this plot.

TABLE 9
Prior odds ratios estimated from 193 CADP1A patients from an
individual marker models, adjusted for age and gender. Ten
of the 15 markers in this model were not selected to continue
to this stage of discovery. Note that the prevalence of disease
in CADP1A is 0.45, while it is 0.33 in the CADP2 group. Additionally,
CADP1A were matched for age and gender.

| | |
|---|---|
| CADP1A | CADP2 |

| Marker | OR | Conf. Int | OR | Conf. Int |
|---|---|---|---|---|
| HSP70 | 1.58 | (1.2, 2.2) | 0.88 | (0.7, 1.1) |
| RBP4 | 0.84 | (0.7, 1.1) | 0.92 | (0.8, 1.1) |
| corin | 0.88 | (0.7, 1.2) | 0.99 | (0.8, 1.2) |
| SERPINA12 | 1.01 | (0.8, 1.3) | 0.86 | (0.7, 1.0) |
| APOB | 1.07 | (0.8, 1.4) | 1.17 | (0.9, 1.5) |

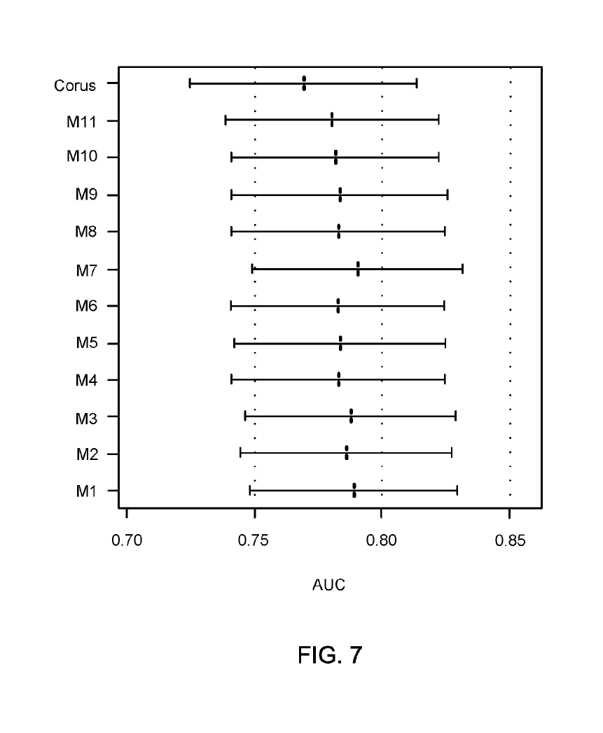

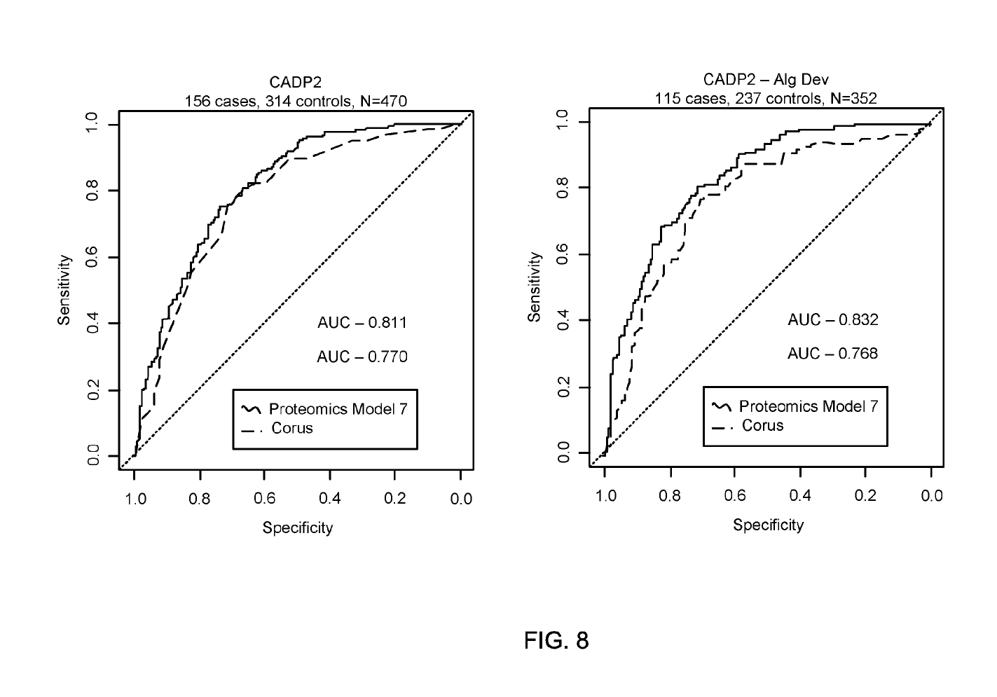

AUC Values and ROC Curves Although Model 7 was selected based on AIC_{c} and not on the basis of its AUC, it does have a superior value in the main model set (see Table 21 and FIGS. 7 and 8), and outperforms Corus, when compared on the full CADP2 data set. This is in the face of a possible upwards bias for Corus. FIG. 7 shows estimates of AUC (area under the curve) values for all main models. All estimates given are adjusted for optimism by bootstrap, except for the Corus model, which was not fitted in this analysis. However, it is worth noting that some of the subjects in this data set were sued for Algorithm Development (model fitting) of the Corus model. FIG. 8 shows plots of the ROC curves for the best proteomics model and the Corus scores on the CADP2 patients (left) and on the same set, after excluding Corus Alg. Dev. Subject (right). Note that while the reported estimates elsewhere in this document for AUC are adjusted for optimism in the proteomics models, these plots are necessarily made of unadjusted values.

Other Model Performance Statistics

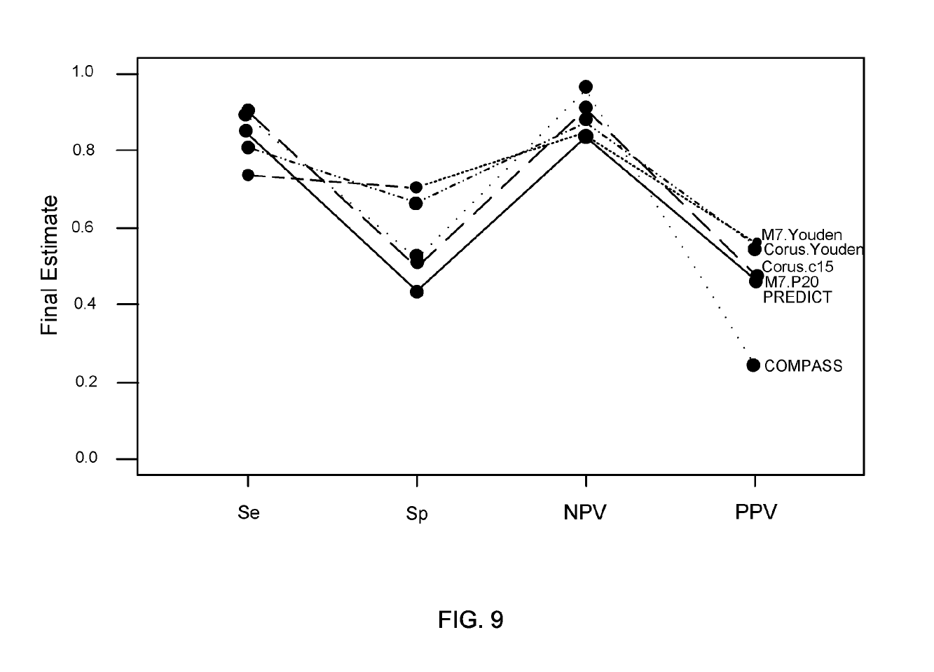

With regards to other measures of model performance such as Sensitivity and Specificity, two cutoffs were considered for the proteomics model. The first was to set the cutoff so that a positive result was all subjects with predicted probabilities >20% of having oCAD >50%. This was the criteria for the cutoff set for the original Corus test. The second cut point examined was the Youden cutoff, which takes the point at which the minimum distance from the upper left corner to the AUC curve occurs. This tends to maximize sensitivity and specificity simultaneously. This was compared to the performance in the same CADP2 patient set using a cutoff of 15 for Corus (Table 10 and FIG. 9). Note that, similar to the AUC estimates, the Se, Sp, NPV, and PPV estimates for Model 7 are optimism adjusted, while the Corus estimate is not adjusted, and therefore might be somewhat positively biased the difference in the PPV for COMPASS is likely related the reduced disease prevalence in this cohort, relative to all others, which are subsets of PREDICT.

FIG. 9 shows relative diagnostic performance measures for Model 7 on CADP2 patients for two cutoffs, compared to performance of Corus on the same patients (Corus.c15), and the published values for Corus in the COMPASS and PREDICT studies.

TABLE 10
Optimism adjusted estimates of the final model for different
cutoffs, compared to the published values of the same statistics
in the COMPASS and PREDICT validation studies. Corus.c15
and Corus.Youden is the performance of Corus in the same
data set as the M7 results (N = 470). The Youden cutoff
was selected from the AUC estimated on this group of patients,
while the Corus.c15 cutoff comes from the algorithm development
phase of Corus.

| Group | Cutoff | Se | Sp | NPV | PPV |
|---|---|---|---|---|---|
| M7.Youden | Pr(oCAD) > 0.35 | 0.74 | 0.70 | 0.84 | 0.55 |
| Corus.Youden | Corus > 20 | 0.81 | 0.67 | 0.87 | 0.55 |
| M7.P20 | Pr(oCAD) > 0.20 | 0.89 | 0.52 | 0.91 | 0.48 |
| Corus.c15 | Corus > 15 | 0.90 | 0.50 | 0.91 | 0.47 |
| COMPASS | Corus > 15 | 0.89 | 0.52 | 0.96 | 0.24 |
| PREDICT | Corus > 15 | 0.85 | 0.43 | 0.83 | 0.46 |

Model 7 Performance on Certain Subsets of Subjects.

After fitting the main model 7 on the entire N=470 CADP2 group of patients, it was then applied to several subsets to compare performance in these groups to Corus. Because the comparison of the model to Corus was the primary goal here, the fitted model was then used to predict the data excluding the subjects with imputed Corus data (referred to as the ‘All’ set). The subsets were then taken from this non-Corus imputed data set. Note that these estimates are unadjusted for optimism, and so are somewhat higher than the actual performance estimates given in the earlier main results. However, both Corus performance and Model 7 performance are calculated on the same subsets of subjects (Table 11).

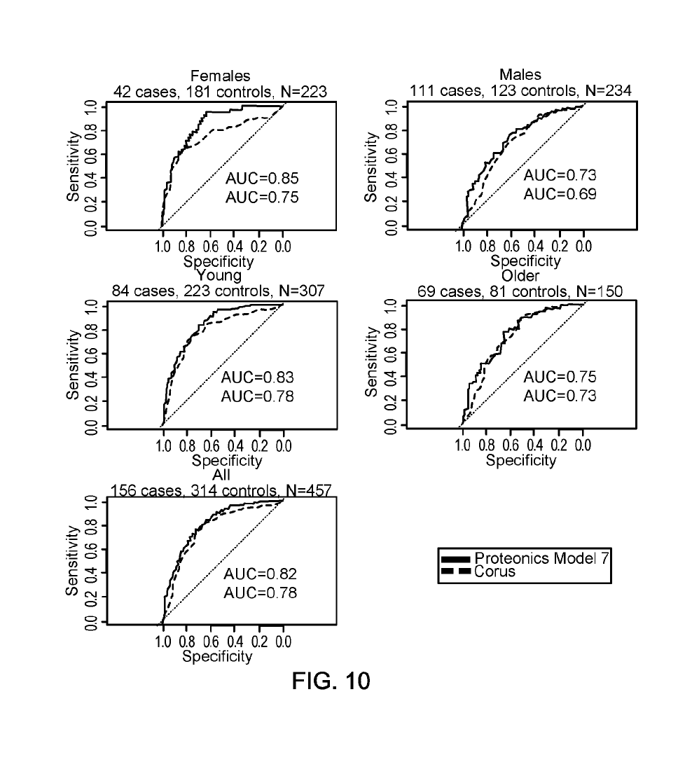

FIG. 10 shows ROC plots comparing the predictive performance of Model 7 and Corus within different subsets of subjects.

TABLE 11
Diagnostic performance in subsets of patients using model 7 fitted
once on the entire set with Corus data (N = 457). Similarly,
the Corus values are using the normal Corus score, and then calculating
performance in each set using the cutoff of 15.

| Group | Model | N | Prev | AUC | Se | Sp | NPV | PPV |
|---|---|---|---|---|---|---|---|---|
| All | Model7 | 457 | 33% | 0.817 | 0.91 | 0.53 | 0.92 | 0.49 |
| All | Corus | 457 | 33% | 0.780 | 0.90 | 0.51 | 0.91 | 0.48 |
| Females | Model7 | 223 | 19% | 0.853 | 0.79 | 0.73 | 0.94 | 0.40 |
| Females | Corus | 223 | 19% | 0.751 | 0.67 | 0.80 | 0.91 | 0.43 |
| Males | Model7 | 234 | 47% | 0.730 | 0.95 | 0.24 | 0.85 | 0.53 |
| Males | Corus | 234 | 47% | 0.690 | 0.99 | 0.09 | 0.92 | 0.50 |
| Young | Model7 | 307 | 27% | 0.835 | 0.86 | 0.65 | 0.92 | 0.48 |
| Young | Corus | 307 | 27% | 0.776 | 0.85 | 0.58 | 0.91 | 0.43 |
| Older | Model7 | 150 | 46% | 0.751 | 0.97 | 0.21 | 0.89 | 0.51 |
| Older | Corus | 150 | 46% | 0.727 | 0.97 | 0.32 | 0.93 | 0.55 |

Exploratory Analyses

Several sets of additional models were run in this analysis for exploratory purposes. The first was a set of models comparing results from the best proteomics model of the main field with a variety of combinations of Corus results on the same subjects (N₁=457).

The second set looked at performance of both Corus and the best proteomics model on the cohort, excluding all subjects used in Corus AlgDev originally (N₂=364). The third set of models looked at the performance of Corus, and the best proteomics model. The fourth set examined the model 7 predictor terms in a proportional odds regression model, performed on the full set of 470 subjects.

Exploratory Set 1: Corus and Proteomics

These models were run on a data set very close to that used for the main models (Table 12). The only difference was that the 13 subjects missing Corus scores were excluded from this exploratory analysis, resulting in a total sample size of 457. In the earlier main model results, Corus scores were imputed for these 13 (see earlier sections for details).

Exploratory Set 2: Excluding AlgDev Samples

Exploratory Set 2 was run on the subjects with available proteomic data that were not originally used in Algorithm Development for Corus (N₂₌₃₆₄). The models are listed in Table 13.

Exploratory Set 3: Proteomics, Corus

Exploratory Set 3 was run on the CADP2A subjects (N₃=176). The models are listed in Table 14.

Exploratory Set 4: Ordinal Regression Exploratory Set 4 was run on the full set of subjects (N₄=470). The model is listed in Table 15.

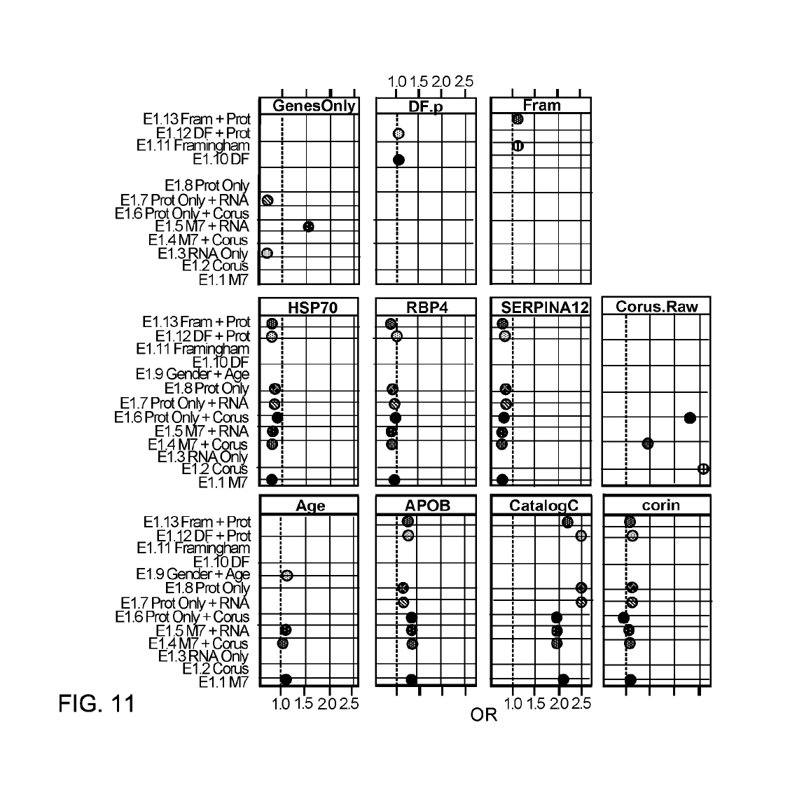

FIG. 11 shows odds ratio estimates for the terms in the exploratory models in Exp1.1 to Exp1.13. The odds ratio for gender is not shown for platting purposes, due to its large size, relative to the other OR.

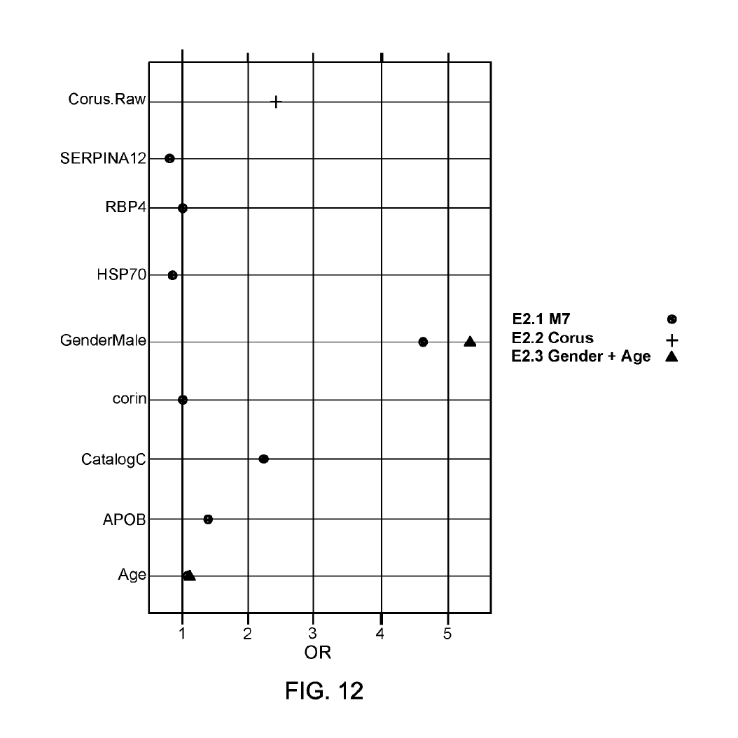

FIG. 12 shows odds ratio estimates for the terms in the exploratory models Exp2.1 to Exp2.3. Note that this data set excludes the Alg Dev subjects.

TABLE 12
The forms of the Exploratory Set 1 models. The response is a binary
variable indicating Case or Control status. ‘Corus Genes Only’
is the Corus score with the male or female intercept subtracted out,
depending on the gender of the subject. ‘Corus’ is the raw Corus
algorithm score. Note that an additional parameter for the Corus term
is estimated in this model from this specific data set. Further note,
for comparability purposes, Model 7 was re-estimated on this data set.

Exploratory Set 1

| | |
|---|---|
| Exploratory Model 1.1 | CAD ~ Model7 |
| Exploratory Model 1.2 | CAD ~ Corus |
| Exploratory Model 1.3 | CAD ~ CorusGenesOnly |
| Exploratory Model 1.4 | CAD ~ Model7 + Corus |
| Exploratory Model 1.5 | CAD ~ Model7 + CorusGenesOnly |
| Exploratory Model 1.6 | CAD ~ Model7 + Corus, except no Age or Sex |
| | in Model 7 |
| Exploratory Model 1.7 | CAD ~ Model7 + CorusGenesOnly, no Age, |
| | Sex |
| Exploratory Model 1.8 | CAD ~ Model7, no Age, Sex |
| Exploratory Model 1.9 | CAD ~ Gender + Age |
| Exploratory Model 1.10 | CAD ~ Diamond-Forrester |
| Exploratory Model 1.11 | CAD ~ FraminghamRiskScore |
| Exploratory Model 1.12 | CAD ~ Diamond-Forrester + |
| | Proteins(Model7 − Sex − Age) |
| Exploratory Model 1.13 | CAD ~ FraminghamRS + Proteins(Model7 − |
| | Sex − Age) |

TABLE 13
The forms of the Exploratory Set 2 models. The response is
a binary variable indicating Case or Control status. ‘Corus’
is the raw Corus algorithm score. Note that an additional
parameter for the Corus term is estimated in this model from
this specific data set. Further note, for comparability purposes,
Model 7 was re-estimated on this data set.

| Exploratory Set 2 | |
|---|---|
| E2.1 | CAD ~ Model7 |
| E2.2 | CAD ~ Corus |
| E2.3 | CAD ~ Gender + Age |

TABLE 14
The forms of the Exploratory Set 3 models. The response is
a binary variable indicating Case or Control status. ‘Corus
Genes Only’ is the Corus score with the male or female
intercept subtracted out, depending on the sex of the subject.
Note that an additional parameter for the Corus term is estimated
in this model from this specific data set.

| Exploratory Set 3 | |
|---|---|
| E3.1 | CAD ~ Model7 |
| E3.2 | CAD ~ Model7 + CorusGenesOnly |

TABLE 15
The form of the Exploratory Set 4 model. The response is an ordinal
variable indicating groups of stenosis (as measured by QCA or clinical
read of 0-24%, 25-49%, 50-69%, 70-99% and 100% occlusion. These
categories were selected to be similar to CTA categories, as well
as to fulfill the assumptions of this type of modelling (different
intercepts for each category with a constant association (slope)
for each predictor among stenosis categories. Further note, Model
7 was re-estimated on this data set of (N = 470).

| Exploratory Set 4 | |
|---|---|
| E4.1 | Ordinal CAD ~ Model7 |

TABLE 16
Unadjusted estimates of diagnostic performance of the exploratory
models. The results given use the cutoff to the predicted probability
of being a case being greater than 20%. Note that Model 7 and Corus
in the full sense both contain Age and Gender terms, while Proteomics
Only and RNA only have no Age or Gender terms in the models. Also
note the models E1.10, E3.5 and E3.6 had predicted probability values
whose smallest values ranged from roughly 20% probability and up,
which led to extreme values for the diagnostic statistics such as
Specificity, which use a cutoff of 20% to be calculated.

| Tag | N | AUC | Se | Sp | NPV | PPV |
|---|---|---|---|---|---|---|
| E1.1 M7 | 457 | 0.82 | 0.91 | 0.53 | 0.92 | 0.49 |
| E1.2 Corus | 457 | 0.78 | 0.90 | 0.50 | 0.91 | 0.48 |
| E1.3 RNA Only | 457 | 0.60 | 0.99 | 0.01 | 0.60 | 0.33 |
| E1.4 M7 + Corus | 457 | 0.82 | 0.92 | 0.55 | 0.93 | 0.51 |
| E1.5 M7 + RNA | 457 | 0.82 | 0.92 | 0.56 | 0.93 | 0.51 |
| E1.6 Prot Only + Corus | 457 | 0.81 | 0.90 | 0.55 | 0.91 | 0.50 |
| E1.7 Prot Only + RNA | 457 | 0.74 | 0.90 | 0.37 | 0.88 | 0.42 |
| E1.8 Prot Only | 457 | 0.72 | 0.89 | 0.33 | 0.86 | 0.40 |
| E1.9 Gender + Age | 457 | 0.78 | 0.90 | 0.44 | 0.90 | 0.45 |
| E1.10 DF | 457 | 0.66 | 1.00 | 0.00 | | 0.33 |
| E1.11 Framingham | 457 | 0.75 | 0.90 | 0.38 | 0.88 | 0.42 |
| E1.12 DF + Prot | 457 | 0.76 | 0.88 | 0.44 | 0.88 | 0.44 |
| E1.13 Fram + Prot | 457 | 0.79 | 0.92 | 0.50 | 0.93 | 0.48 |
| E2.1 M7 | 352 | 0.83 | 0.91 | 0.57 | 0.93 | 0.50 |
| E2.2 Corus | 352 | 0.77 | 0.88 | 0.50 | 0.89 | 0.46 |
| E2.3 Gender + Age | 352 | 0.78 | 0.88 | 0.47 | 0.89 | 0.44 |
| E3.1 M7 | 176 | 0.73 | 0.98 | 0.08 | 0.78 | 0.52 |
| E3.2 M7 + RNA | 176 | 0.75 | 0.99 | 0.20 | 0.94 | 0.56 |
| E4.1 M7 Ordinal Reg. | 470 | 0.80 | 0.96 | 0.39 | 0.95 | 0.44 |

Model 7 Calibration and Discrimination

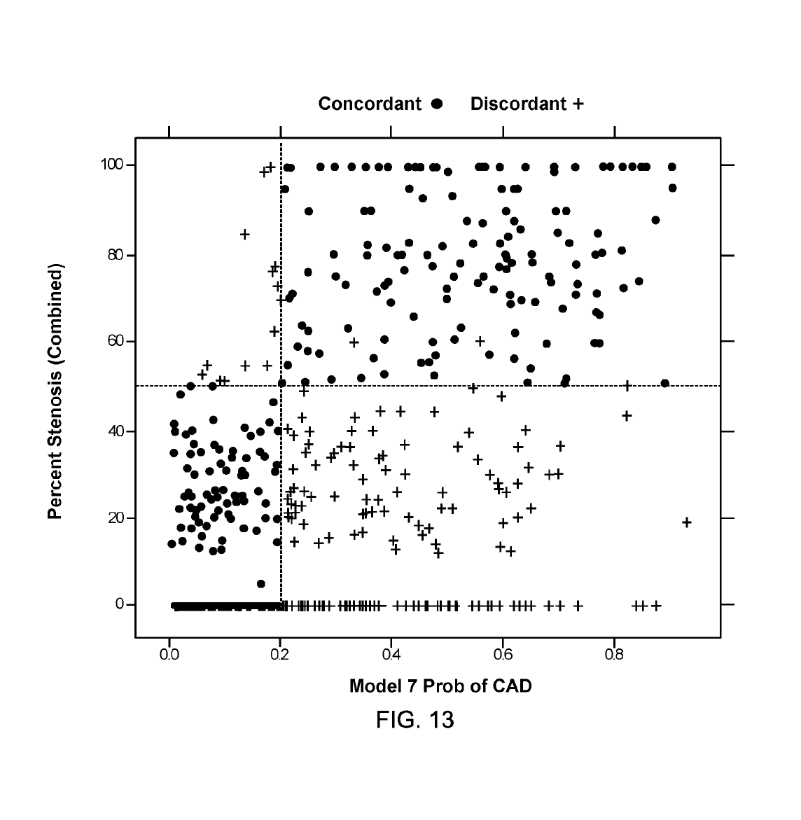

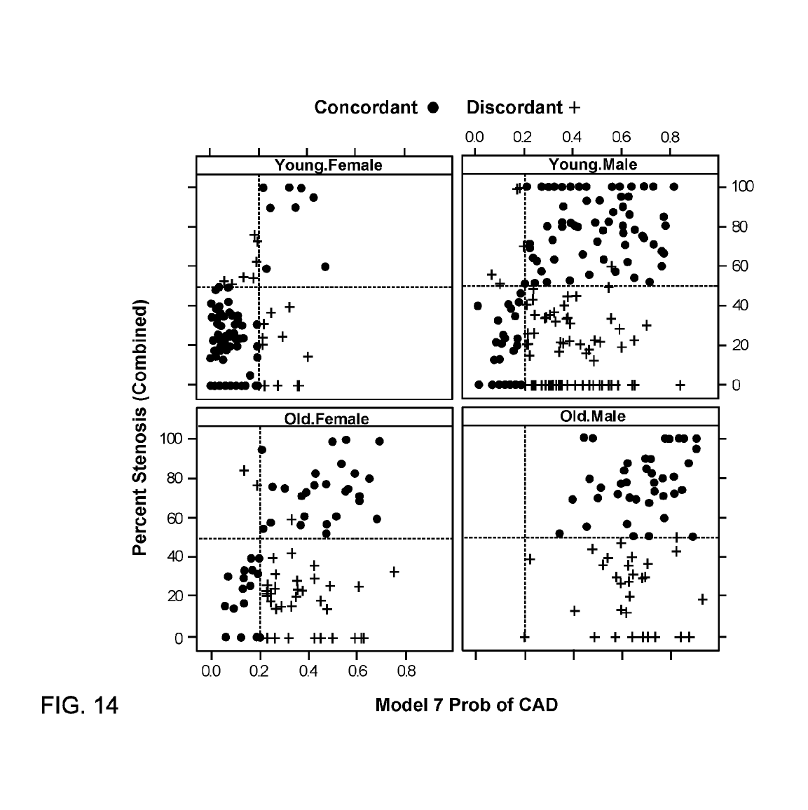

A comparison of the predicted values for the Model 7 results to the stenosis of the patient can be seen in FIGS. 13 and 14.

FIG. 13 shows a comparison of predicted values from Model 7 to the percent stenosis of the same patient. Points are colored by agreement of model 7 to reference status.

FIG. 14 shows comparison of predicted values from Model 7 to the percent stenosis of the same patient. Points are colored by agreement of model 7 to reference status.

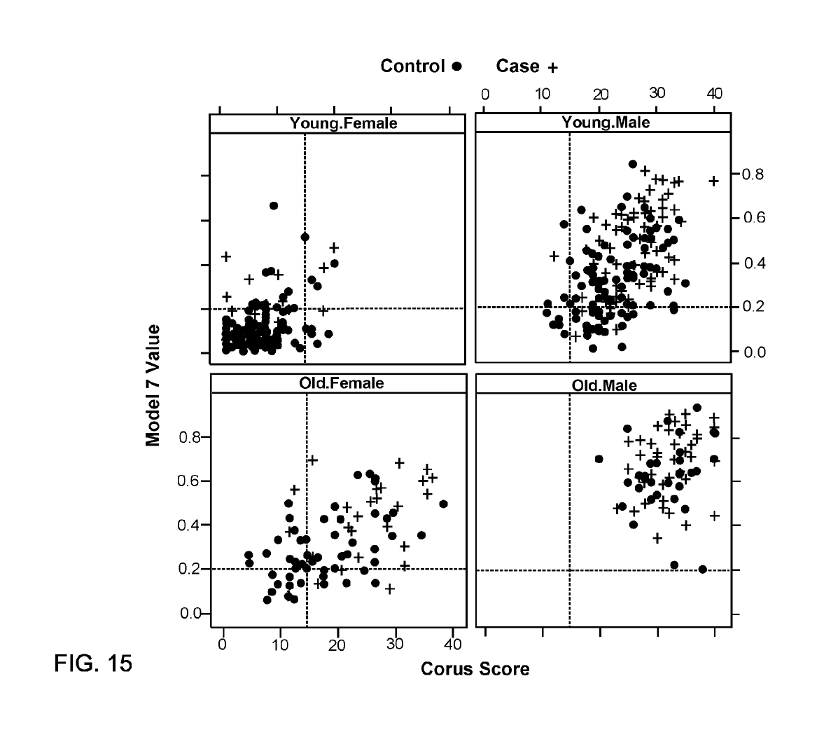

A comparison of the predicted values for the Corus RNA expression-based test and the Model 7 results can be seen in FIG. 15. FIG. 15 shows comparison of predicted values from Model 7 to predicted values from the Corus score run on the same sample. Points are colored by true reference status. The dashed lines indicate the cutoff of 20% for Model 7 and the cutoff of 15 for Corus. Both models predict all males 65 and over to be cases. However, there are two controls in this group that are borderline near the 20% cutoff used for Model 7, with predicted values of 0.223 and 0.202, respectively. No female under the age of 65 has a higher predicted value than 48%. Model 7 is better at discriminating Young Female cases than Corus, in this data set, while Corus does slightly better with Young Male cases in this analysis.

TABLE 17
Summary of incorrect calls using the Model 7 predicted values
with a 20% cutoff. Also given is the interquartile range of observed
percent stenosis for those subjects called incorrectly.

| DemogGroup | RefGroup | NiPctofTotal | NumWrong PctWrong | Sten25 | Sten75 |
|---|---|---|---|---|---|
| Old.Female | Control | 55/84 | 36 | 0 | 26 |
| Old.Female | Case | 29/84 | 2 | 79 | 83 |
| Old.Male | Control | 30/72 | 30 | 0 | 36 |
| Old.Male | Case | 42/72 | 0 | | |
| Young.Female | Control | 129/144 | 12 | 0 | 27 |
| Young.Female | Case | 15/144 | 7 | 54 | 68 |
| Young.Male | Control | 100/170 | 70 | 0 | 31 |
| Young.Male | Case | 70/170 | 5 | 55 | 99 |

### APPENDIX

TABLE 18
Pairwise marker rank correlations, as shown
in the heatmap, expressed as a percentage.

| | corin | HSP70 | PlGF | Sex | Corus | A8MPO | A12TNF | Age |
|---|---|---|---|---|---|---|---|---|
| SERPINA12 | 13 | 15 | 8 | −6 | −2 | 6 | 24 | 4 |
| corin | | 20 | 10 | 11 | 8 | 6 | 8 | −1 |
| HSP70 | | | 7 | 10 | 6 | 28 | 8 | 5 |
| PlGF | | | | 7 | 10 | 10 | 8 | 4 |
| Sex | | | | | 67 | 15 | 10 | −3 |
| Corus.Tfm | | | | | | 16 | 16 | 53 |
| A8MPO | | | | | | | 34 | 3 |
| A12TNF | | | | | | | | 1 |
| Age | | | | | | | | |
| CPVar | | | | | | | | |
| NTproBNP | | | | | | | | |
| Adiponectin | | | | | | | | |
| APOA1 | | | | | | | | |
| SmokeVar | | | | | | | | |
| DyslipVar | | | | | | | | |
| APOB | | | | | | | | |
| RBP4 | | | | | | | | |

| | CP | NTproBNP | Adip | APOA1 | Smoke | Dyslip | APOB | RBP4 |
|---|---|---|---|---|---|---|---|---|
| SERPINA12 | −4 | −4 | −1 | −2 | −5 | 1 | 5 | 8 |
| corin | −7 | −3 | −5 | 1 | −13 | −13 | 8 | 9 |
| HSP70 | −2 | 4 | −3 | −3 | −19 | −6 | −3 | 4 |
| PlGF | −2 | 14 | −7 | −4 | 5 | −4 | 4 | 2 |
| Sex | −6 | −9 | −22 | −25 | −30 | −49 | 9 | |
| Corus.Tfm | 27 | 12 | −3 | −13 | −33 | −55 | −3 | −3 |
| A8MPO | −7 | −3 | −12 | −2 | −9 | −12 | −1 | 2 |
| A12TNF | −1 | −6 | | −11 | −4 | −12 | 3 | 4 |
| Age | 46 | 32 | 23 | 12 | −22 | −24 | −13 | −1 |
| CPVar | | 12 | 13 | 7 | −12 | −19 | −8 | |
| NTproBNP | | | 33 | 17 | −2 | 4 | −10 | −6 |
| Adiponectin | | | | 48 | 7 | 8 | −7 | −3 |
| APOA1 | | | | | | 13 | −5 | 2 |
| SmokeVar | | | | | | 33 | 5 | 6 |
| DyslipVar | | | | | | | 3 | 9 |
| APOB | | | | | | | | 11 |
| RBP4 | | | | | | | | |

TABLE 19
Model fit measurements in the form of AIC and corrected
AIC for the main models. Values are the median
value across all bootstrap iterations.

| Model | AIC | AICc |
|---|---|---|
| M1 | 482.98 | 486.91 |
| M2 | 483.31 | 488.23 |
| M3 | 483.41 | 489.05 |
| M4 | 481.14 | 488.91 |
| M5 | 483.39 | 491.79 |
| M6 | 479.01 | 489.09 |
| M7 | 481.55 | 485.04 |
| M8 | 481.38 | 489.04 |
| M9 | 477.13 | 486.36 |
| M10 | 489.56 | 492.45 |
| M11 | 483.45 | 489.73 |

TABLE 20
Listing of Odds Ratios for coefficients of Model 7, fitted
to the full CADP2 data set (N = 470). Lower and Upper
are the 95% confidence intervals for the Odds Ratio.

| Term Order | Model | Lower | Odds Ratio | Upper | pValue |
|---|---|---|---|---|---|
| GenderMale | M7 | 2.38 | 3.84 | 6.31 | <0.001 |
| CatalogC | M7 | 1.63 | 2.14 | 2.84 | <0.001 |
| NTproBNP | CatalogC | 1.24 | 1.55 | 1.95 | <0.001 |
| APOB | M7 | 1.03 | 1.30 | 1.65 | 0.028 |
| A12TNF | CatalogC | 0.98 | 1.21 | 1.51 | 0.075 |
| PlGF | CatalogC | 0.98 | 1.20 | 1.49 | 0.083 |
| A8MPO | CatalogC | 0.96 | 1.19 | 1.48 | 0.107 |
| Age | M7 | 1.05 | 1.07 | 1.10 | <0.001 |
| corin | M7 | 0.81 | 1.03 | 1.31 | 0.813 |
| RBP4 | M7 | 0.79 | 0.95 | 1.14 | 0.557 |
| HSP70 | M7 | 0.70 | 0.88 | 1.10 | 0.266 |
| SERPINA12 | M7 | 0.68 | 0.81 | 0.96 | 0.017 |
| AdipA1 | CatalogC | 0.48 | 0.62 | 0.80 | <0.001 |

TABLE 21
Optimism corrected estimates of AUC values for all main models.
These values come from the final model fitted on the full CADP2
data set. Note that Corus was not fitted on this data (for
this analysis), and is not optimism adjusted so there may be
some slight inflation in its performance, due to the presence
of some Alg Dev subjects in this data set.

| Model | Lower | AUC | Upper |
|---|---|---|---|
| M1 | 0.748 | 0.789 | 0.830 |
| M2 | 0.744 | 0.785 | 0.827 |
| M3 | 0.746 | 0.788 | 0.829 |
| M4 | 0.741 | 0.783 | 0.824 |
| M5 | 0.742 | 0.783 | 0.825 |
| M6 | 0.741 | 0.783 | 0.825 |
| M7 | 0.750 | 0.791 | 0.831 |
| M8 | 0.741 | 0.783 | 0.825 |
| M9 | 0.742 | 0.783 | 0.825 |
| M10 | 0.741 | 0.782 | 0.823 |
| M11 | 0.739 | 0.781 | 0.822 |
| Corus | 0.725 | 0.770 | 0.814 |

TABLE 22
Listing of Odds Ratio estimates from logistic regression exploratory models.

| ModelName | Tag | Term | OR | Lower | Upper | pVal |
|---|---|---|---|---|---|---|
| Exp1.1 | E1.1 M7 | (Intercept) | 0.00 | 0.00 | 0.02 | 0.0000 |
| Exp1.1 | E1.1 M7 | SERPINA12 | 0.80 | 0.67 | 0.95 | 0.0140 |
| Exp1.1 | E1.1 M7 | HSP70 | 0.88 | 0.69 | 1.11 | 0.2762 |
| Exp1.1 | E1.1 M7 | RBP4 | 0.96 | 0.79 | 1.15 | 0.6389 |
| Exp1.1 | E1.1 M7 | corin | 1.05 | 0.82 | 1.34 | 0.6990 |
| Exp1.1 | E1.1 M7 | Age | 1.08 | 1.05 | 1.10 | 0.0000 |
| Exp1.1 | E1.1 M7 | APOB | 1.30 | 1.03 | 1.66 | 0.0318 |
| Exp1.1 | E1.1 M7 | CatalogC | 2.11 | 1.61 | 2.82 | 0.0000 |
| Exp1.1 | E1.1 M7 | GenderMale | 4.30 | 2.64 | 7.16 | 0.0000 |
| Exp1.10 | E1.10 DF | (Intercept) | 0.26 | 0.19 | 0.36 | 0.0000 |
| Exp1.10 | E1.10 DF | DF.p | 1.02 | 1.01 | 1.02 | 0.0000 |
| Exp1.11 | E1.11 Framingham | (Intercept) | 0.17 | 0.11 | 0.24 | 0.0000 |
| Exp1.11 | E1.11 Framingham | Fram | 1.12 | 1.09 | 1.16 | 0.0000 |
| Exp1.12 | E1.12 DF + Prot | (Intercept) | 0.48 | 0.33 | 0.68 | 0.0000 |
| Exp1.12 | E1.12 DF + Prot | SERPINA12 | 0.86 | 0.73 | 1.01 | 0.0668 |
| Exp1.12 | E1.12 DF + Prot | HSP70 | 0.89 | 0.72 | 1.11 | 0.3011 |
| Exp1.12 | E1.12 DF + Prot | RBP4 | 0.99 | 0.83 | 1.18 | 0.8888 |
| Exp1.12 | E1.12 DF + Prot | DF.p | 1.02 | 1.01 | 1.02 | 0.0000 |
| Exp1.12 | E1.12 DF + Prot | corin | 1.10 | 0.88 | 1.38 | 0.3914 |
| Exp1.12 | E1.12 DF + Prot | APOB | 1.18 | 0.95 | 1.47 | 0.1351 |
| Exp1.12 | E1.12 DF + Prot | CatalogC | 2.51 | 1.94 | 3.29 | 0.0000 |
| Exp1.13 | E1.13 Fram + Prot | (Intercept) | 0.31 | 0.20 | 0.47 | 0.0000 |
| Exp1.13 | E1.13 Fram + Prot | SERPINA12 | 0.82 | 0.69 | 0.97 | 0.0238 |
| Exp1.13 | E1.13 Fram + Prot | HSP70 | 0.89 | 0.71 | 1.11 | 0.3201 |
| Exp1.13 | E1.13 Fram + Prot | RBP4 | 0.92 | 0.76 | 1.10 | 0.3543 |
| Exp1.13 | E1.13 Fram + Prot | corin | 1.07 | 0.85 | 1.35 | 0.5657 |
| Exp1.13 | E1.13 Fram + Prot | Fram | 1.10 | 1.07 | 1.14 | 0.0000 |
| Exp1.13 | E1.13 Fram + Prot | APOB | 1.21 | 0.97 | 1.51 | 0.0911 |
| Exp1.13 | E1.13 Fram + Prot | CatalogC | 2.16 | 1.65 | 2.86 | 0.0000 |
| Exp1.2 | E1.2 Corus | (Intercept) | 0.91 | 0.72 | 1.16 | 0.4539 |
| Exp1.2 | E1.2 Corus | Corus.Raw | 2.61 | 2.12 | 3.28 | 0.0000 |
| Exp1.3 | E1.3 RNA Only | (Intercept) | 0.08 | 0.02 | 0.26 | 0.0000 |

Tables 23-25: Listing of Odds Ratio estimates from logistic regression exploratory models

| ModelName | Tag | Term | OR | Lower | Upper | pVal |
|---|---|---|---|---|---|---|
| Exp1.3 | E1.3 RNA Only | GenesOnly | 0.68 | 0.53 | 0.87 | 0.0021 |
| Exp1.4 | E1.4 M7 + Corus | (Intercept) | 0.03 | 0.00 | 0.42 | 0.0105 |
| Exp1.4 | E1.4 M7 + Corus | SERPINA12 | 0.80 | 0.67 | 0.95 | 0.0145 |
| Exp1.4 | E1.4 M7 + Corus | HSP70 | 0.90 | 0.71 | 1.15 | 0.4111 |
| Exp1.4 | E1.4 M7 + Corus | RBP4 | 0.96 | 0.79 | 1.16 | 0.6517 |
| Exp1.4 | E1.4 M7 + Corus | corin | 1.02 | 0.80 | 1.31 | 0.8608 |
| Exp1.4 | E1.4 M7 + Corus | Age | 1.05 | 1.02 | 1.09 | 0.0053 |
| Exp1.4 | E1.4 M7 + Corus | APOB | 1.29 | 1.02 | 1.65 | 0.0335 |
| Exp1.4 | E1.4 M7 + Corus | Corus.Raw | 1.47 | 0.96 | 2.25 | 0.0750 |
| Exp1.4 | E1.4 M7 + Corus | CatalogC | 2.04 | 1.55 | 2.73 | 0.0000 |
| Exp1.4 | E1.4 M7 + Corus | GenderMale | 2.33 | 1.03 | 5.39 | 0.0448 |
| Exp1.5 | E1.5 M7 + RNA | (Intercept) | 0.02 | 0.00 | 0.24 | 0.0023 |
| Exp1.5 | E1.5 M7 + RNA | SERPINA12 | 0.80 | 0.67 | 0.95 | 0.0144 |
| Exp1.5 | E1.5 M7 + RNA | HSP70 | 0.90 | 0.71 | 1.14 | 0.3910 |
| Exp1.5 | E1.5 M7 + RNA | RBP4 | 0.96 | 0.79 | 1.15 | 0.6324 |
| Exp1.5 | E1.5 M7 + RNA | corin | 1.04 | 0.81 | 1.34 | 0.7331 |
| Exp1.5 | E1.5 M7 + RNA | Age | 1.08 | 1.05 | 1.10 | 0.0000 |
| Exp1.5 | E1.5 M7 + RNA | APOB | 1.29 | 1.02 | 1.64 | 0.0365 |
| Exp1.5 | E1.5 M7 + RNA | GenesOnly | 1.50 | 0.94 | 2.42 | 0.0944 |
| Exp1.5 | E1.5 M7 + RNA | CatalogC | 2.04 | 1.55 | 2.73 | 0.0000 |
| Exp1.5 | E1.5 M7 + RNA | GenderMale | 7.35 | 3.31 | 16.88 | 0.0000 |
| Exp1.6 | E1.6 Prot Only + Corus | SERPINA12 | 0.82 | 0.69 | 0.97 | 0.0218 |
| Exp1.6 | E1.6 Prot Only + Corus | HSP70 | 0.94 | 0.75 | 1.19 | 0.6303 |
| Exp1.6 | E1.6 Prot Only + Corus | RBP4 | 0.97 | 0.80 | 1.16 | 0.7180 |
| Exp1.6 | E1.6 Prot Only + Corus | corin | 0.99 | 0.77 | 1.26 | 0.9330 |
| Exp1.6 | E1.6 Prot Only + Corus | APOB | 1.24 | 0.99 | 1.55 | 0.0640 |
| Exp1.6 | E1.6 Prot Only + Corus | (Intercept) | 1.30 | 0.99 | 1.74 | 0.0651 |
| Exp1.6 | E1.6 Prot Only + Corus | CatalogC | 2.02 | 1.54 | 2.70 | 0.0000 |
| Exp1.6 | E1.6 Prot Only + Corus | Corus.Raw | 2.36 | 1.89 | 3.00 | 0.0000 |
| Exp1.7 | E1.7 Prot Only + RNA | (Intercept) | 0.17 | 0.04 | 0.60 | 0.0067 |
| Exp1.7 | E1.7 Prot Only + RNA | GenesOnly | 0.70 | 0.54 | 0.92 | 0.0095 |
| Exp1.7 | E1.7 Prot Only + RNA | SERPINA12 | 0.84 | 0.71 | 0.99 | 0.0392 |

| ModelName | Tag | Term | OR | Lower | Upper | pVal |
|---|---|---|---|---|---|---|
| Exp1.7 | E1.7 Prot Only + RNA | HSP70 | 0.89 | 0.72 | 1.10 | 0.2807 |
| Exp1.7 | E1.7 Prot Only + RNA | RBP4 | 0.97 | 0.81 | 1.15 | 0.7036 |
| Exp1.7 | E1.7 Prot Only + RNA | corin | 1.07 | 0.85 | 1.33 | 0.5611 |
| Exp1.7 | E1.7 Prot Only + RNA | APOB | 1.16 | 0.94 | 1.43 | 0.1813 |
| Exp1.7 | E1.7 Prot Only + RNA | CatalogC | 2.60 | 2.01 | 3.41 | 0.0000 |
| Exp1.8 | E1.8 Prot Only | SERPINA12 | 0.84 | 0.71 | 0.98 | 0.0337 |
| Exp1.8 | E1.8 Prot Only | (Intercept) | 0.89 | 0.70 | 1.15 | 0.3761 |
| Exp1.8 | E1.8 Prot Only | HSP70 | 0.92 | 0.74 | 1.13 | 0.4182 |
| Exp1.8 | E1.8 Prot Only | RBP4 | 0.96 | 0.81 | 1.14 | 0.6496 |
| Exp1.8 | E1.8 Prot Only | corin | 1.09 | 0.87 | 1.36 | 0.4487 |
| Exp1.8 | E1.8 Prot Only | APOB | 1.17 | 0.95 | 1.45 | 0.1343 |
| Exp1.8 | E1.8 Prot Only | CatalogC | 2.60 | 2.02 | 3.40 | 0.0000 |
| Exp1.9 | E1.9 Gender + Age | (Intercept) | 0.00 | 0.00 | 0.01 | 0.0000 |
| Exp1.9 | E1.9 Gender + Age | Age | 1.08 | 1.06 | 1.10 | 0.0000 |
| Exp1.9 | E1.9 Gender + Age | GenderMale | 5.17 | 3.27 | 8.36 | 0.0000 |
| Exp2.1 | E2.1 M7 | (Intercept) | 0.01 | 0.00 | 0.03 | 0.0000 |
| Exp2.1 | E2.1 M7 | SERPINA12 | 0.84 | 0.69 | 1.01 | 0.0663 |
| Exp2.1 | E2.1 M7 | HSP70 | 0.84 | 0.64 | 1.10 | 0.2139 |
| Exp2.1 | E2.1 M7 | RBP4 | 1.03 | 0.84 | 1.26 | 0.8070 |
| Exp2.1 | E2.1 M7 | corin | 1.03 | 0.77 | 1.35 | 0.8586 |
| Exp2.1 | E2.1 M7 | Age | 1.07 | 1.04 | 1.10 | 0.0000 |
| Exp2.1 | E2.1 M7 | APOB | 1.37 | 1.05 | 1.80 | 0.0237 |
| Exp2.1 | E2.1 M7 | CatalogC | 2.20 | 1.64 | 3.02 | 0.0000 |
| Exp2.1 | E2.1 M7 | GenderMale | 4.62 | 2.66 | 8.20 | 0.0000 |
| Exp2.2 | E2.2 Corus | (Intercept) | 0.90 | 0.68 | 1.18 | 0.4383 |
| Exp2.2 | E2.2 Corus | Corus.Raw | 2.39 | 1.91 | 3.04 | 0.0000 |
| Exp2.3 | E2.3 Gender + Age | (Intercept) | 0.00 | 0.00 | 0.01 | 0.0000 |
| Exp2.3 | E2.3 Gender + Age | Age | 1.07 | 1.05 | 1.10 | 0.0000 |
| Exp2.3 | E2.3 Gender + Age | GenderMale | 5.34 | 3.20 | 9.11 | 0.0000 |
| Exp3.1 | E3.1 M7 | (Intercept) | 0.04 | 0.00 | 0.48 | 0.0123 |
| Exp3.1 | E3.1 M7 | SERPINA12 | 0.90 | 0.71 | 1.13 | 0.3610 |
| Exp3.1 | E3.1 M7 | corin | 0.96 | 0.68 | 1.37 | 0.8184 |

| ModelName | Tag | Term | OR | Lower | Upper | pVal |
|---|---|---|---|---|---|---|
| Exp3.1 | E3.1 M7 | RBP4 | 0.98 | 0.73 | 1.31 | 0.8949 |
| Exp3.1 | E3.1 M7 | Age | 1.05 | 1.01 | 1.09 | 0.0155 |
| Exp3.1 | E3.1 M7 | HSP70 | 1.09 | 0.76 | 1.57 | 0.6497 |
| Exp3.1 | E3.1 M7 | APOB | 1.37 | 0.96 | 2.00 | 0.0900 |
| Exp3.1 | E3.1 M7 | GenderMale | 1.88 | 0.91 | 3.96 | 0.0901 |
| Exp3.1 | E3.1 M7 | CatalogC | 3.10 | 1.89 | 5.32 | 0.0000 |
| Exp3.2 | E3.2 M7 + RNA | SERPINA12 | 0.91 | 0.72 | 1.14 | 0.4112 |
| Exp3.2 | E3.2 M7 + RNA | corin | 0.91 | 0.64 | 1.31 | 0.6223 |
| Exp3.2 | E3.2 M7 + RNA | RBP4 | 0.98 | 0.73 | 1.33 | 0.9069 |
| Exp3.2 | E3.2 M7 + RNA | Age | 1.04 | 1.00 | 1.08 | 0.0322 |
| Exp3.2 | E3.2 M7 + RNA | HSP70 | 1.16 | 0.80 | 1.68 | 0.4406 |
| Exp3.2 | E3.2 M7 + RNA | APOB | 1.40 | 0.97 | 2.05 | 0.0781 |
| Exp3.2 | E3.2 M7 + RNA | (Intercept) | 1.50 | 0.03 | 81.37 | 0.8393 |
| Exp3.2 | E3.2 M7 + RNA | GenesOnly | 2.29 | 1.15 | 4.78 | 0.0218 |
| Exp3.2 | E3.2 M7 + RNA | CatalogC | 2.94 | 1.78 | 5.09 | 0.0000 |
| Exp3.2 | E3.2 M7 + RNA | GenderMale | 5.73 | 1.76 | 20.14 | 0.0048 |

Final Model 7 Equation

For a logistic regression model where logit{CAD=1|X}=Cβ, the final fitted equation is:

X\hat{β}=−5.29177+1.34519*I(Sex=Male)+0.6996(Age)+0.76010(Set1)+0.02924(corin)+0.26173(APOB)−0.12978(HSP70)−0.05482(RBP4)−0.20628(SERPINA12)

Set1=−0.38017Batch−0.47149AdipA1+0.43946NTproBNP+0.18471PlGF+0.17573A8MPO+0.19449A12TNF

where I(Sex=Male) is an indicator function that is 1 if the subject is Male and 0 otherwise, Age is expressed in years, and the protein marker values are transformed as log 2 (Calculated Concentration+2). Set 1 is the predictor function from a nested logistic regression model with the same response variable, that is logit {CAD=1|X}, where Xi are different predictors than the full model, as listed in the equations above. There are several terms that are the means of two protein assays within a patient, these include AdipA1 (mean of Adiponectin+APOA1), A8MPO (mean of S100A8 and MPO), and A12T N F (mean of S100A12 and TNFAIP6).

### Example 3: Subtractive Analysis of Markers from Model 7

This example provides results of a subtractive analysis where all possible subsets of the full model of interest were run using logistic regression:

High Level: logit{Pr(obstructive CAD)}=Intercept+Age+Sex+APOB+corin+HSP70+RBP4+SERPINA12+Lower Level Model Fitted Value

Lower Level: logit{Pr(obstructive CAD)}=Intercept+AdipA1+NTproBNP+P1GF+A8MPO+A12TNF,

where AdipA1 is the mean of Adiponectin and APOA1, A8MPO is the mean of S100A8 and MPO and A12TNF is the mean of S100A12 and TNFAIP6.

Each new model created via the subtractive analysis was a logistic regression model, which was fitted using an iteratively reweighted least squares method. Each time a new model was fit, this method calculated the coefficients or “weights” of the terms that minimize the least squares criteria for that specific model. For each particular model, these can vary due to the presence/absence of particular terms and the amount of information they each give about the response variable.

Two measures of model performance for each new sub-model were collected: AICc, Akaike's Information Criteria (corrected for the number of cases in the model fitting set; here AICc=AIC+{2p(p+1)/n−p−1}, where p is the number of parameters in the model and n is the number of cases used in model fitting. n=156), and AUC (area under the curve). For the AICc, the smaller the value, the better the model captures the information in the data set, while for the AUC, the larger the value, the better the model correctly classifies patients as having or not having obstructive CAD. AUC is the area under the ROC curve, which was calculated in the standard way, but is generally a rank ordered statistic, which is the probability for all possible (case, control) pairs that the model correctly orders the case as a higher risk of disease than the control.

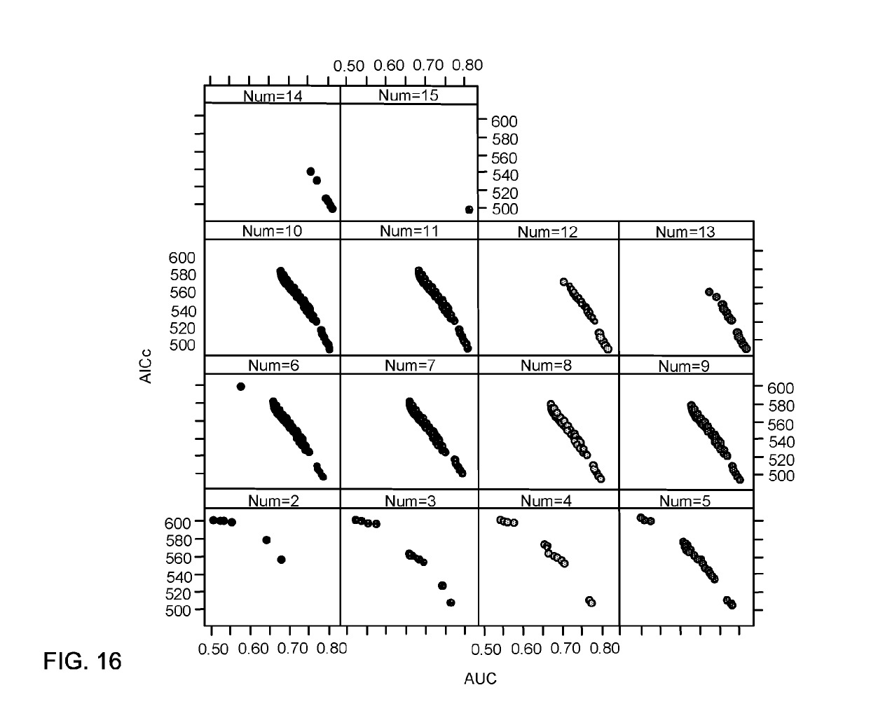

The AICc and the AUC were calculated after the models were fit, where the coefficient values were determined. They were calculated in the same way for all models. As such, they are generally, relatively comparable across all models, despite the differences in the specific terms used in each model as part of the subtractive analysis. The individual models and values of AICc and AUC for each model are given in Table 26A-B. In total, 4094 distinct, new models were generated and tested for this example. FIG. 16 shows the ability of a given model to explain the variation in the data (AIC) compared to the ability of the model to correctly classify the patients for obstructive CAD (AUC) for the number of markers in the given model (moving sequentially from 15 to 1 markers).

TABLE 26A

| | HighLevel |
|---|---|
| 1 | CAD ~ Intercept.High + Sex + Age + APOB + corin + HSP70 + RBP4 + SERPINA12 + LowLevel |
| 2 | CAD ~ Intercept.High + Age + APOB + corin + HSP70 + RBP4 + SERPINA12 + LowLevel |
| 3 | CAD ~ Intercept.High + Sex + Age + APOB + corin + HSP70 + RBP4 + SERPINA12 + LowLevel |
| 4 | CAD ~ Intercept.High + Sex + Age + APOB + corin + HSP70 + RBP4 + SERPINA12 + LowLevel |
| 5 | CAD ~ Intercept.High + Sex + Age + APOB + corin + HSP70 + RBP4 + SERPINA12 + LowLevel |
| 6 | CAD ~ Intercept.High + Sex + Age + APOB + corin + HSP70 + RBP4 + SERPINA12 + LowLevel |
| 7 | CAD ~ Intercept.High + Sex + Age + APOB + corin + HSP70 + RBP4 + SERPINA12 + LowLevel |
| 8 | CAD ~ Intercept.High + Sex + Age + APOB + corin + HSP70 + RBP4 + LowLevel |
| 9 | CAD ~ Intercept.High + Sex + Age + APOB + corin + HSP70 + SERPINA12 + LowLevel |
| 10 | CAD ~ Intercept.High + Sex + Age + APOB + corin + RBP4 + SERPINA12 + LowLevel |
| 11 | CAD ~ Intercept.High + Sex + Age + APOB + HSP70 + RBP4 + SERPINA12 + LowLevel |
| 12 | CAD ~ Intercept.High + Sex + Age + corin + HSP70 + RBP4 + SERPINA12 + LowLevel |
| 13 | CAD ~ Intercept.High + Sex + APOB + corin + HSP70 + RBP4 + SERPINA12 + LowLevel |
| 14 | CAD ~ Intercept.High + Age + APOB + corin + HSP70 + RBP4 + SERPINA12 + LowLevel |
| 15 | CAD ~ Intercept.High + Age + APOB + corin + HSP70 + RBP4 + SERPINA12 + LowLevel |
| 16 | CAD ~ Intercept.High + Age + APOB + corin + HSP70 + RBP4 + SERPINA12 + LowLevel |
| 17 | CAD ~ Intercept.High + Age + APOB + corin + HSP70 + RBP4 + SERPINA12 + LowLevel |
| 18 | CAD ~ Intercept.High + Age + APOB + corin + HSP70 + RBP4 + SERPINA12 + LowLevel |
| 19 | CAD ~ Intercept.High + Age + APOB + corin + HSP70 + RBP4 + LowLevel |
| 20 | CAD ~ Intercept.High + Age + APOB + corin + HSP70 + SERPINA12 + LowLevel |
| 21 | CAD ~ Intercept.High + Age + APOB + corin + RBP4 + SERPINA12 + LowLevel |
| 22 | CAD ~ Intercept.High + Age + APOB + HSP70 + RBP4 + SERPINA12 + LowLevel |
| 23 | CAD ~ Intercept.High + Age + corin + HSP70 + RBP4 + SERPINA12 + LowLevel |
| 24 | CAD ~ Intercept.High + APOB + corin + HSP70 + RBP4 + SERPINA12 + LowLevel |
| 25 | CAD ~ Intercept.High + Sex + Age + APOB + corin + HSP70 + RBP4 + SERPINA12 + LowLevel |
| 26 | CAD ~ Intercept.High + Sex + Age + APOB + corin + HSP70 + RBP4 + SERPINA12 + LowLevel |
| 27 | CAD ~ Intercept.High + Sex + Age + APOB + corin + HSP70 + RBP4 + SERPINA12 + LowLevel |
| 28 | CAD ~ Intercept.High + Sex + Age + APOB + corin + HSP70 + RBP4 + SERPINA12 + LowLevel |
| 29 | CAD ~ Intercept.High + Sex + Age + APOB + corin + HSP70 + RBP4 + SERPINA12 + LowLevel |
| 30 | CAD ~ Intercept.High + Sex + Age + APOB + corin + HSP70 + RBP4 + SERPINA12 + LowLevel |
| 31 | CAD ~ Intercept.High + Sex + Age + APOB + corin + HSP70 + RBP4 + SERPINA12 + LowLevel |
| 32 | CAD ~ Intercept.High + Sex + Age + APOB + corin + HSP70 + RBP4 + SERPINA12 + LowLevel |
| 33 | CAD ~ Intercept.High + Sex + Age + APOB + corin + HSP70 + RBP4 + SERPINA12 + LowLevel |
| 34 | CAD ~ Intercept.High + Sex + Age + APOB + corin + HSP70 + RBP4 + SERPINA12 + LowLevel |
| 35 | CAD ~ Intercept.High + Sex + Age + APOB + corin + HSP70 + RBP4 + LowLevel |
| 36 | CAD ~ Intercept.High + Sex + Age + APOB + corin + HSP70 + RBP4 + LowLevel |
| 37 | CAD ~ Intercept.High + Sex + Age + APOB + corin + HSP70 + RBP4 + LowLevel |
| 38 | CAD ~ Intercept.High + Sex + Age + APOB + corin + HSP70 + RBP4 + LowLevel |
| 39 | CAD ~ Intercept.High + Sex + Age + APOB + corin + HSP70 + RBP4 + LowLevel |
| 40 | CAD ~ Intercept.High + Sex + Age + APOB + corin + HSP70 + SERPINA12 + LowLevel |
| 41 | CAD ~ Intercept.High + Sex + Age + APOB + corin + HSP70 + SERPINA12 + LowLevel |
| 42 | CAD ~ Intercept.High + Sex + Age + APOB + corin + HSP70 + SERPINA12 + LowLevel |
| 43 | CAD ~ Intercept.High + Sex + Age + APOB + corin + HSP70 + SERPINA12 + LowLevel |
| 44 | CAD ~ Intercept.High + Sex + Age + APOB + corin + HSP70 + SERPINA12 + LowLevel |
| 45 | CAD ~ Intercept.High + Sex + Age + APOB + corin + HSP70 + LowLevel |
| 46 | CAD ~ Intercept.High + Sex + Age + APOB + corin + RBP4 + SERPINA12 + LowLevel |
| 47 | CAD ~ Intercept.High + Sex + Age + APOB + corin + RBP4 + SERPINA12 + LowLevel |
| 48 | CAD ~ Intercept.High + Sex + Age + APOB + corin + RBP4 + SERPINA12 + LowLevel |
| 49 | CAD ~ Intercept.High + Sex + Age + APOB + corin + RBP4 + SERPINA12 + LowLevel |
| 50 | CAD ~ Intercept.High + Sex + Age + APOB + corin + RBP4 + SERPINA12 + LowLevel |
| 51 | CAD ~ Intercept.High + Sex + Age + APOB + corin + RBP4 + LowLevel |
| 52 | CAD ~ Intercept.High + Sex + Age + APOB + corin + SERPINA12 + LowLevel |
| 53 | CAD ~ Intercept.High + Sex + Age + APOB + HSP70 + RBP4 + SERPINA12 + LowLevel |
| 54 | CAD ~ Intercept.High + Sex + Age + APOB + HSP70 + RBP4 + SERPINA12 + LowLevel |
| 55 | CAD ~ Intercept.High + Sex + Age + APOB + HSP70 + RBP4 + SERPINA12 + LowLevel |
| 56 | CAD ~ Intercept.High + Sex + Age + APOB + HSP70 + RBP4 + SERPINA12 + LowLevel |
| 57 | CAD ~ Intercept.High + Sex + Age + APOB + HSP70 + RBP4 + SERPINA12 + LowLevel |
| 58 | CAD ~ Intercept.High + Sex + Age + APOB + HSP70 + RBP4 + LowLevel |
| 59 | CAD ~ Intercept.High + Sex + Age + APOB + HSP70 + SERPINA12 + LowLevel |
| 60 | CAD ~ Intercept.High + Sex + Age + APOB + RBP4 + SERPINA12 + LowLevel |
| 61 | CAD ~ Intercept.High + Sex + Age + corin + HSP70 + RBP4 + SERPINA12 + LowLevel |
| 62 | CAD ~ Intercept.High + Sex + Age + corin + HSP70 + RBP4 + SERPINA12 + LowLevel |
| 63 | CAD ~ Intercept.High + Sex + Age + corin + HSP70 + RBP4 + SERPINA12 + LowLevel |
| 64 | CAD ~ Intercept.High + Sex + Age + corin + HSP70 + RBP4 + SERPINA12 + LowLevel |
| 65 | CAD ~ Intercept.High + Sex + Age + corin + HSP70 + RBP4 + SERPINA12 + LowLevel |
| 66 | CAD ~ Intercept.High + Sex + Age + corin + HSP70 + RBP4 + LowLevel |
| 67 | CAD ~ Intercept.High + Sex + Age + corin + HSP70 + SERPINA12 + LowLevel |
| 68 | CAD ~ Intercept.High + Sex + Age + corin + RBP4 + SERPINA12 + LowLevel |
| 69 | CAD ~ Intercept.High + Sex + Age + HSP70 + RBP4 + SERPINA12 + LowLevel |
| 70 | CAD ~ Intercept.High + Sex + APOB + corin + HSP70 + RBP4 + SERPINA12 + LowLevel |
| 71 | CAD ~ Intercept.High + Sex + APOB + corin + HSP70 + RBP4 + SERPINA12 + LowLevel |
| 72 | CAD ~ Intercept.High + Sex + APOB + corin + HSP70 + RBP4 + SERPINA12 + LowLevel |
| 73 | CAD ~ Intercept.High + Sex + APOB + corin + HSP70 + RBP4 + SERPINA12 + LowLevel |
| 74 | CAD ~ Intercept.High + Sex + APOB + corin + HSP70 + RBP4 + SERPINA12 + LowLevel |
| 75 | CAD ~ Intercept.High + Sex + APOB + corin + HSP70 + RBP4 + LowLevel |
| 76 | CAD ~ Intercept.High + Sex + APOB + corin + HSP70 + SERPINA12 + LowLevel |
| 77 | CAD ~ Intercept.High + Sex + APOB + corin + RBP4 + SERPINA12 + LowLevel |
| 78 | CAD ~ Intercept.High + Sex + APOB + HSP70 + RBP4 + SERPINA12 + LowLevel |
| 79 | CAD ~ Intercept.High + Sex + corin + HSP70 + RBP4 + SERPINA12 + LowLevel |
| 80 | CAD ~ Intercept.High + Age + APOB + corin + HSP70 + RBP4 + SERPINA12 + LowLevel |
| 81 | CAD ~ Intercept.High + Age + APOB + corin + HSP70 + RBP4 + SERPINA12 + LowLevel |
| 82 | CAD ~ Intercept.High + Age + APOB + corin + HSP70 + RBP4 + SERPINA12 + LowLevel |
| 83 | CAD ~ Intercept.High + Age + APOB + corin + HSP70 + RBP4 + SERPINA12 + LowLevel |
| 84 | CAD ~ Intercept.High + Age + APOB + corin + HSP70 + RBP4 + SERPINA12 + LowLevel |
| 85 | CAD ~ Intercept.High + Age + APOB + corin + HSP70 + RBP4 + SERPINA12 + LowLevel |
| 86 | CAD ~ Intercept.High + Age + APOB + corin + HSP70 + RBP4 + SERPINA12 + LowLevel |
| 87 | CAD ~ Intercept.High + Age + APOB + corin + HSP70 + RBP4 + SERPINA12 + LowLevel |
| 88 | CAD ~ Intercept.High + Age + APOB + corin + HSP70 + RBP4 + SERPINA12 + LowLevel |
| 89 | CAD ~ Intercept.High + Age + APOB + corin + HSP70 + RBP4 + SERPINA12 + LowLevel |
| 90 | CAD ~ Intercept.High + Age + APOB + corin + HSP70 + RBP4 + LowLevel |
| 91 | CAD ~ Intercept.High + Age + APOB + corin + HSP70 + RBP4 + LowLevel |
| 92 | CAD ~ Intercept.High + Age + APOB + corin + HSP70 + RBP4 + LowLevel |
| 93 | CAD ~ Intercept.High + Age + APOB + corin + HSP70 + RBP4 + LowLevel |
| 94 | CAD ~ Intercept.High + Age + APOB + corin + HSP70 + RBP4 + LowLevel |
| 95 | CAD ~ Intercept.High + Age + APOB + corin + HSP70 + SERPINA12 + LowLevel |
| 96 | CAD ~ Intercept.High + Age + APOB + corin + HSP70 + SERPINA12 + LowLevel |
| 97 | CAD ~ Intercept.High + Age + APOB + corin + HSP70 + SERPINA12 + LowLevel |
| 98 | CAD ~ Intercept.High + Age + APOB + corin + HSP70 + SERPINA12 + LowLevel |
| 99 | CAD ~ Intercept.High + Age + APOB + corin + HSP70 + SERPINA12 + LowLevel |
| 100 | CAD ~ Intercept.High + Age + APOB + corin + HSP70 + LowLevel |
| 101 | CAD ~ Intercept.High + Age + APOB + corin + RBP4 + SERPINA12 + LowLevel |
| 102 | CAD ~ Intercept.High + Age + APOB + corin + RBP4 + SERPINA12 + LowLevel |
| 103 | CAD ~ Intercept.High + Age + APOB + corin + RBP4 + SERPINA12 + LowLevel |
| 104 | CAD ~ Intercept.High + Age + APOB + corin + RBP4 + SERPINA12 + LowLevel |
| 105 | CAD ~ Intercept.High + Age + APOB + corin + RBP4 + SERPINA12 + LowLevel |
| 106 | CAD ~ Intercept.High + Age + APOB + corin + RBP4 + LowLevel |
| 107 | CAD ~ Intercept.High + Age + APOB + corin + SERPINA12 + LowLevel |
| 108 | CAD ~ Intercept.High + Age + APOB + HSP70 + RBP4 + SERPINA12 + LowLevel |
| 109 | CAD ~ Intercept.High + Age + APOB + HSP70 + RBP4 + SERPINA12 + LowLevel |
| 110 | CAD ~ Intercept.High + Age + APOB + HSP70 + RBP4 + SERPINA12 + LowLevel |
| 111 | CAD ~ Intercept.High + Age + APOB + HSP70 + RBP4 + SERPINA12 + LowLevel |
| 112 | CAD ~ Intercept.High + Age + APOB + HSP70 + RBP4 + SERPINA12 + LowLevel |
| 113 | CAD ~ Intercept.High + Age + APOB + HSP70 + RBP4 + LowLevel |
| 114 | CAD ~ Intercept.High + Age + APOB + HSP70 + SERPINA12 + LowLevel |
| 115 | CAD ~ Intercept.High + Age + APOB + RBP4 + SERPINA12 + LowLevel |
| 116 | CAD ~ Intercept.High + Age + corin + HSP70 + RBP4 + SERPINA12 + LowLevel |
| 117 | CAD ~ Intercept.High + Age + corin + HSP70 + RBP4 + SERPINA12 + LowLevel |
| 118 | CAD ~ Intercept.High + Age + corin + HSP70 + RBP4 + SERPINA12 + LowLevel |
| 119 | CAD ~ Intercept.High + Age + corin + HSP70 + RBP4 + SERPINA12 + LowLevel |
| 120 | CAD ~ Intercept.High + Age + corin + HSP70 + RBP4 + SERPINA12 + LowLevel |
| 121 | CAD ~ Intercept.High + Age + corin + HSP70 + RBP4 + LowLevel |
| 122 | CAD ~ Intercept.High + Age + corin + HSP70 + SERPINA12 + LowLevel |
| 123 | CAD ~ Intercept.High + Age + corin + RBP4 + SERPINA12 + LowLevel |
| 124 | CAD ~ Intercept.High + Age + HSP70 + RBP4 + SERPINA12 + LowLevel |
| 125 | CAD ~ Intercept.High + APOB + corin + HSP70 + RBP4 + SERPINA12 + LowLevel |
| 126 | CAD ~ Intercept.High + APOB + corin + HSP70 + RBP4 + SERPINA12 + LowLevel |
| 127 | CAD ~ Intercept.High + APOB + corin + HSP70 + RBP4 + SERPINA12 + LowLevel |
| 128 | CAD ~ Intercept.High + APOB + corin + HSP70 + RBP4 + SERPINA12 + LowLevel |
| 129 | CAD ~ Intercept.High + APOB + corin + HSP70 + RBP4 + SERPINA12 + LowLevel |
| 130 | CAD ~ Intercept.High + APOB + corin + HSP70 + RBP4 + LowLevel |
| 131 | CAD ~ Intercept.High + APOB + corin + HSP70 + SERPINA12 + LowLevel |
| 132 | CAD ~ Intercept.High + APOB + corin + RBP4 + SERPINA12 + LowLevel |
| 133 | CAD ~ Intercept.High + APOB + HSP70 + RBP4 + SERPINA12 + LowLevel |
| 134 | CAD ~ Intercept.High + corin + HSP70 + RBP4 + SERPINA12 + LowLevel |
| 135 | CAD ~ Intercept.High + Sex + Age + APOB + corin + HSP70 + RBP4 + SERPINA12 + LowLevel |
| 136 | CAD ~ Intercept.High + Sex + Age + APOB + corin + HSP70 + RBP4 + SERPINA12 + LowLevel |
| 137 | CAD ~ Intercept.High + Sex + Age + APOB + corin + HSP70 + RBP4 + SERPINA12 + LowLevel |
| 138 | CAD ~ Intercept.High + Sex + Age + APOB + corin + HSP70 + RBP4 + SERPINA12 + LowLevel |
| 139 | CAD ~ Intercept.High + Sex + Age + APOB + corin + HSP70 + RBP4 + SERPINA12 + LowLevel |
| 140 | CAD ~ Intercept.High + Sex + Age + APOB + corin + HSP70 + RBP4 + SERPINA12 + LowLevel |
| 141 | CAD ~ Intercept.High + Sex + Age + APOB + corin + HSP70 + RBP4 + SERPINA12 + LowLevel |
| 142 | CAD ~ Intercept.High + Sex + Age + APOB + corin + HSP70 + RBP4 + SERPINA12 + LowLevel |
| 143 | CAD ~ Intercept.High + Sex + Age + APOB + corin + HSP70 + RBP4 + SERPINA12 + LowLevel |
| 144 | CAD ~ Intercept.High + Sex + Age + APOB + corin + HSP70 + RBP4 + SERPINA12 + LowLevel |
| 145 | CAD ~ Intercept.High + Sex + Age + APOB + corin + HSP70 + RBP4 + LowLevel |
| 146 | CAD ~ Intercept.High + Sex + Age + APOB + corin + HSP70 + RBP4 + LowLevel |
| 147 | CAD ~ Intercept.High + Sex + Age + APOB + corin + HSP70 + RBP4 + LowLevel |
| 148 | CAD ~ Intercept.High + Sex + Age + APOB + corin + HSP70 + RBP4 + LowLevel |
| 149 | CAD ~ Intercept.High + Sex + Age + APOB + corin + HSP70 + RBP4 + LowLevel |
| 150 | CAD ~ Intercept.High + Sex + Age + APOB + corin + HSP70 + RBP4 + LowLevel |
| 151 | CAD ~ Intercept.High + Sex + Age + APOB + corin + HSP70 + RBP4 + LowLevel |
| 152 | CAD ~ Intercept.High + Sex + Age + APOB + corin + HSP70 + RBP4 + LowLevel |
| 153 | CAD ~ Intercept.High + Sex + Age + APOB + corin + HSP70 + RBP4 + LowLevel |
| 154 | CAD ~ Intercept.High + Sex + Age + APOB + corin + HSP70 + RBP4 + LowLevel |
| 155 | CAD ~ Intercept.High + Sex + Age + APOB + corin + HSP70 + SERPINA12 + LowLevel |
| 156 | CAD ~ Intercept.High + Sex + Age + APOB + corin + HSP70 + SERPINA12 + LowLevel |
| 157 | CAD ~ Intercept.High + Sex + Age + APOB + corin + HSP70 + SERPINA12 + LowLevel |
| 158 | CAD ~ Intercept.High + Sex + Age + APOB + corin + HSP70 + SERPINA12 + LowLevel |
| 159 | CAD ~ Intercept.High + Sex + Age + APOB + corin + HSP70 + SERPINA12 + LowLevel |
| 160 | CAD ~ Intercept.High + Sex + Age + APOB + corin + HSP70 + SERPINA12 + LowLevel |
| 161 | CAD ~ Intercept.High + Sex + Age + APOB + corin + HSP70 + SERPINA12 + LowLevel |
| 162 | CAD ~ Intercept.High + Sex + Age + APOB + corin + HSP70 + SERPINA12 + LowLevel |
| 163 | CAD ~ Intercept.High + Sex + Age + APOB + corin + HSP70 + SERPINA12 + LowLevel |
| 164 | CAD ~ Intercept.High + Sex + Age + APOB + corin + HSP70 + SERPINA12 + LowLevel |
| 165 | CAD ~ Intercept.High + Sex + Age + APOB + corin + HSP70 + LowLevel |
| 166 | CAD ~ Intercept.High + Sex + Age + APOB + corin + HSP70 + LowLevel |
| 167 | CAD ~ Intercept.High + Sex + Age + APOB + corin + HSP70 + LowLevel |
| 168 | CAD ~ Intercept.High + Sex + Age + APOB + corin + HSP70 + LowLevel |
| 169 | CAD ~ Intercept.High + Sex + Age + APOB + corin + HSP70 + LowLevel |
| 170 | CAD ~ Intercept.High + Sex + Age + APOB + corin + RBP4 + SERPINA12 + LowLevel |
| 171 | CAD ~ Intercept.High + Sex + Age + APOB + corin + RBP4 + SERPINA12 + LowLevel |
| 172 | CAD ~ Intercept.High + Sex + Age + APOB + corin + RBP4 + SERPINA12 + LowLevel |
| 173 | CAD ~ Intercept.High + Sex + Age + APOB + corin + RBP4 + SERPINA12 + LowLevel |
| 174 | CAD ~ Intercept.High + Sex + Age + APOB + corin + RBP4 + SERPINA12 + LowLevel |
| 175 | CAD ~ Intercept.High + Sex + Age + APOB + corin + RBP4 + SERPINA12 + LowLevel |
| 176 | CAD ~ Intercept.High + Sex + Age + APOB + corin + RBP4 + SERPINA12 + LowLevel |
| 177 | CAD ~ Intercept.High + Sex + Age + APOB + corin + RBP4 + SERPINA12 + LowLevel |
| 178 | CAD ~ Intercept.High + Sex + Age + APOB + corin + RBP4 + SERPINA12 + LowLevel |
| 179 | CAD ~ Intercept.High + Sex + Age + APOB + corin + RBP4 + SERPINA12 + LowLevel |
| 180 | CAD ~ Intercept.High + Sex + Age + APOB + corin + RBP4 + LowLevel |
| 181 | CAD ~ Intercept.High + Sex + Age + APOB + corin + RBP4 + LowLevel |
| 182 | CAD ~ Intercept.High + Sex + Age + APOB + corin + RBP4 + LowLevel |
| 183 | CAD ~ Intercept.High + Sex + Age + APOB + corin + RBP4 + LowLevel |
| 184 | CAD ~ Intercept.High + Sex + Age + APOB + corin + RBP4 + LowLevel |
| 185 | CAD ~ Intercept.High + Sex + Age + APOB + corin + SERPINA12 + LowLevel |
| 186 | CAD ~ Intercept.High + Sex + Age + APOB + corin + SERPINA12 + LowLevel |
| 187 | CAD ~ Intercept.High + Sex + Age + APOB + corin + SERPINA12 + LowLevel |
| 188 | CAD ~ Intercept.High + Sex + Age + APOB + corin + SERPINA12 + LowLevel |
| 189 | CAD ~ Intercept.High + Sex + Age + APOB + corin + SERPINA12 + LowLevel |
| 190 | CAD ~ Intercept.High + Sex + Age + APOB + corin + LowLevel |
| 191 | CAD ~ Intercept.High + Sex + Age + APOB + HSP70 + RBP4 + SERPINA12 + LowLevel |
| 192 | CAD ~ Intercept.High + Sex + Age + APOB + HSP70 + RBP4 + SERPINA12 + LowLevel |
| 193 | CAD ~ Intercept.High + Sex + Age + APOB + HSP70 + RBP4 + SERPINA12 + LowLevel |
| 194 | CAD ~ Intercept.High + Sex + Age + APOB + HSP70 + RBP4 + SERPINA12 + LowLevel |
| 195 | CAD ~ Intercept.High + Sex + Age + APOB + HSP70 + RBP4 + SERPINA12 + LowLevel |
| 196 | CAD ~ Intercept.High + Sex + Age + APOB + HSP70 + RBP4 + SERPINA12 + LowLevel |
| 197 | CAD ~ Intercept.High + Sex + Age + APOB + HSP70 + RBP4 + SERPINA12 + LowLevel |
| 198 | CAD ~ Intercept.High + Sex + Age + APOB + HSP70 + RBP4 + SERPINA12 + LowLevel |
| 199 | CAD ~ Intercept.High + Sex + Age + APOB + HSP70 + RBP4 + SERPINA12 + LowLevel |
| 200 | CAD ~ Intercept.High + Sex + Age + APOB + HSP70 + RBP4 + SERPINA12 + LowLevel |
| 201 | CAD ~ Intercept.High + Sex + Age + APOB + HSP70 + RBP4 + LowLevel |
| 202 | CAD ~ Intercept.High + Sex + Age + APOB + HSP70 + RBP4 + LowLevel |
| 203 | CAD ~ Intercept.High + Sex + Age + APOB + HSP70 + RBP4 + LowLevel |
| 204 | CAD ~ Intercept.High + Sex + Age + APOB + HSP70 + RBP4 + LowLevel |
| 205 | CAD ~ Intercept.High + Sex + Age + APOB + HSP70 + RBP4 + LowLevel |
| 206 | CAD ~ Intercept.High + Sex + Age + APOB + HSP70 + SERPINA12 + LowLevel |
| 207 | CAD ~ Intercept.High + Sex + Age + APOB + HSP70 + SERPINA12 + LowLevel |
| 208 | CAD ~ Intercept.High + Sex + Age + APOB + HSP70 + SERPINA12 + LowLevel |
| 209 | CAD ~ Intercept.High + Sex + Age + APOB + HSP70 + SERPINA12 + LowLevel |
| 210 | CAD ~ Intercept.High + Sex + Age + APOB + HSP70 + SERPINA12 + LowLevel |
| 211 | CAD ~ Intercept.High + Sex + Age + APOB + HSP70 + LowLevel |
| 212 | CAD ~ Intercept.High + Sex + Age + APOB + RBP4 + SERPINA12 + LowLevel |
| 213 | CAD ~ Intercept.High + Sex + Age + APOB + RBP4 + SERPINA12 + LowLevel |
| 214 | CAD ~ Intercept.High + Sex + Age + APOB + RBP4 + SERPINA12 + LowLevel |
| 215 | CAD ~ Intercept.High + Sex + Age + APOB + RBP4 + SERPINA12 + LowLevel |
| 216 | CAD ~ Intercept.High + Sex + Age + APOB + RBP4 + SERPINA12 + LowLevel |
| 217 | CAD ~ Intercept.High + Sex + Age + APOB + RBP4 + LowLevel |
| 218 | CAD ~ Intercept.High + Sex + Age + APOB + SERPINA12 + LowLevel |
| 219 | CAD ~ Intercept.High + Sex + Age + corin + HSP70 + RBP4 + SERPINA12 + LowLevel |
| 220 | CAD ~ Intercept.High + Sex + Age + corin + HSP70 + RBP4 + SERPINA12 + LowLevel |
| 221 | CAD ~ Intercept.High + Sex + Age + corin + HSP70 + RBP4 + SERPINA12 + LowLevel |
| 222 | CAD ~ Intercept.High + Sex + Age + corin + HSP70 + RBP4 + SERPINA12 + LowLevel |
| 223 | CAD ~ Intercept.High + Sex + Age + corin + HSP70 + RBP4 + SERPINA12 + LowLevel |
| 224 | CAD ~ Intercept.High + Sex + Age + corin + HSP70 + RBP4 + SERPINA12 + LowLevel |
| 225 | CAD ~ Intercept.High + Sex + Age + corin + HSP70 + RBP4 + SERPINA12 + LowLevel |
| 226 | CAD ~ Intercept.High + Sex + Age + corin + HSP70 + RBP4 + SERPINA12 + LowLevel |
| 227 | CAD ~ Intercept.High + Sex + Age + corin + HSP70 + RBP4 + SERPINA12 + LowLevel |
| 228 | CAD ~ Intercept.High + Sex + Age + corin + HSP70 + RBP4 + SERPINA12 + LowLevel |
| 229 | CAD ~ Intercept.High + Sex + Age + corin + HSP70 + RBP4 + LowLevel |
| 230 | CAD ~ Intercept.High + Sex + Age + corin + HSP70 + RBP4 + LowLevel |
| 231 | CAD ~ Intercept.High + Sex + Age + corin + HSP70 + RBP4 + LowLevel |
| 232 | CAD ~ Intercept.High + Sex + Age + corin + HSP70 + RBP4 + LowLevel |
| 233 | CAD ~ Intercept.High + Sex + Age + corin + HSP70 + RBP4 + LowLevel |
| 234 | CAD ~ Intercept.High + Sex + Age + corin + HSP70 + SERPINA12 + LowLevel |
| 235 | CAD ~ Intercept.High + Sex + Age + corin + HSP70 + SERPINA12 + LowLevel |
| 236 | CAD ~ Intercept.High + Sex + Age + corin + HSP70 + SERPINA12 + LowLevel |
| 237 | CAD ~ Intercept.High + Sex + Age + corin + HSP70 + SERPINA12 + LowLevel |
| 238 | CAD ~ Intercept.High + Sex + Age + corin + HSP70 + SERPINA12 + LowLevel |
| 239 | CAD ~ Intercept.High + Sex + Age + corin + HSP70 + LowLevel |
| 240 | CAD ~ Intercept.High + Sex + Age + corin + RBP4 + SERPINA12 + LowLevel |
| 241 | CAD ~ Intercept.High + Sex + Age + corin + RBP4 + SERPINA12 + LowLevel |
| 242 | CAD ~ Intercept.High + Sex + Age + corin + RBP4 + SERPINA12 + LowLevel |
| 243 | CAD ~ Intercept.High + Sex + Age + corin + RBP4 + SERPINA12 + LowLevel |
| 244 | CAD ~ Intercept.High + Sex + Age + corin + RBP4 + SERPINA12 + LowLevel |
| 245 | CAD ~ Intercept.High + Sex + Age + corin + RBP4 + LowLevel |
| 246 | CAD ~ Intercept.High + Sex + Age + corin + SERPINA12 + LowLevel |
| 247 | CAD ~ Intercept.High + Sex + Age + HSP70 + RBP4 + SERPINA12 + LowLevel |
| 248 | CAD ~ Intercept.High + Sex + Age + HSP70 + RBP4 + SERPINA12 + LowLevel |
| 249 | CAD ~ Intercept.High + Sex + Age + HSP70 + RBP4 + SERPINA12 + LowLevel |
| 250 | CAD ~ Intercept.High + Sex + Age + HSP70 + RBP4 + SERPINA12 + LowLevel |
| 251 | CAD ~ Intercept.High + Sex + Age + HSP70 + RBP4 + SERPINA12 + LowLevel |
| 252 | CAD ~ Intercept.High + Sex + Age + HSP70 + RBP4 + LowLevel |
| 253 | CAD ~ Intercept.High + Sex + Age + HSP70 + SERPINA12 + LowLevel |
| 254 | CAD ~ Intercept.High + Sex + Age + RBP4 + SERPINA12 + LowLevel |
| 255 | CAD ~ Intercept.High + Sex + APOB + corin + HSP70 + RBP4 + SERPINA12 + LowLevel |
| 256 | CAD ~ Intercept.High + Sex + APOB + corin + HSP70 + RBP4 + SERPINA12 + LowLevel |
| 257 | CAD ~ Intercept.High + Sex + APOB + corin + HSP70 + RBP4 + SERPINA12 + LowLevel |
| 258 | CAD ~ Intercept.High + Sex + APOB + corin + HSP70 + RBP4 + SERPINA12 + LowLevel |
| 259 | CAD ~ Intercept.High + Sex + APOB + corin + HSP70 + RBP4 + SERPINA12 + LowLevel |
| 260 | CAD ~ Intercept.High + Sex + APOB + corin + HSP70 + RBP4 + SERPINA12 + LowLevel |
| 261 | CAD ~ Intercept.High + Sex + APOB + corin + HSP70 + RBP4 + SERPINA12 + LowLevel |
| 262 | CAD ~ Intercept.High + Sex + APOB + corin + HSP70 + RBP4 + SERPINA12 + LowLevel |
| 263 | CAD ~ Intercept.High + Sex + APOB + corin + HSP70 + RBP4 + SERPINA12 + LowLevel |
| 264 | CAD ~ Intercept.High + Sex + APOB + corin + HSP70 + RBP4 + SERPINA12 + LowLevel |
| 265 | CAD ~ Intercept.High + Sex + APOB + corin + HSP70 + RBP4 + LowLevel |
| 266 | CAD ~ Intercept.High + Sex + APOB + corin + HSP70 + RBP4 + LowLevel |
| 267 | CAD ~ Intercept.High + Sex + APOB + corin + HSP70 + RBP4 + LowLevel |
| 268 | CAD ~ Intercept.High + Sex + APOB + corin + HSP70 + RBP4 + LowLevel |
| 269 | CAD ~ Intercept.High + Sex + APOB + corin + HSP70 + RBP4 + LowLevel |
| 270 | CAD ~ Intercept.High + Sex + APOB + corin + HSP70 + SERPINA12 + LowLevel |
| 271 | CAD ~ Intercept.High + Sex + APOB + corin + HSP70 + SERPINA12 + LowLevel |
| 272 | CAD ~ Intercept.High + Sex + APOB + corin + HSP70 + SERPINA12 + LowLevel |
| 273 | CAD ~ Intercept.High + Sex + APOB + corin + HSP70 + SERPINA12 + LowLevel |
| 274 | CAD ~ Intercept.High + Sex + APOB + corin + HSP70 + SERPINA12 + LowLevel |
| 275 | CAD ~ Intercept.High + Sex + APOB + corin + HSP70 + LowLevel |
| 276 | CAD ~ Intercept.High + Sex + APOB + corin + RBP4 + SERPINA12 + LowLevel |
| 277 | CAD ~ Intercept.High + Sex + APOB + corin + RBP4 + SERPINA12 + LowLevel |
| 278 | CAD ~ Intercept.High + Sex + APOB + corin + RBP4 + SERPINA12 + LowLevel |
| 279 | CAD ~ Intercept.High + Sex + APOB + corin + RBP4 + SERPINA12 + LowLevel |
| 280 | CAD ~ Intercept.High + Sex + APOB + corin + RBP4 + SERPINA12 + LowLevel |
| 281 | CAD ~ Intercept.High + Sex + APOB + corin + RBP4 + LowLevel |
| 282 | CAD ~ Intercept.High + Sex + APOB + corin + SERPINA12 + LowLevel |
| 283 | CAD ~ Intercept.High + Sex + APOB + HSP70 + RBP4 + SERPINA12 + LowLevel |
| 284 | CAD ~ Intercept.High + Sex + APOB + HSP70 + RBP4 + SERPINA12 + LowLevel |
| 285 | CAD ~ Intercept.High + Sex + APOB + HSP70 + RBP4 + SERPINA12 + LowLevel |
| 286 | CAD ~ Intercept.High + Sex + APOB + HSP70 + RBP4 + SERPINA12 + LowLevel |
| 287 | CAD ~ Intercept.High + Sex + APOB + HSP70 + RBP4 + SERPINA12 + LowLevel |
| 288 | CAD ~ Intercept.High + Sex + APOB + HSP70 + RBP4 + LowLevel |
| 289 | CAD ~ Intercept.High + Sex + APOB + HSP70 + SERPINA12 + LowLevel |
| 290 | CAD ~ Intercept.High + Sex + APOB + RBP4 + SERPINA12 + LowLevel |
| 291 | CAD ~ Intercept.High + Sex + corin + HSP70 + RBP4 + SERPINA12 + LowLevel |
| 292 | CAD ~ Intercept.High + Sex + corin + HSP70 + RBP4 + SERPINA12 + LowLevel |
| 293 | CAD ~ Intercept.High + Sex + corin + HSP70 + RBP4 + SERPINA12 + LowLevel |
| 294 | CAD ~ Intercept.High + Sex + corin + HSP70 + RBP4 + SERPINA12 + LowLevel |
| 295 | CAD ~ Intercept.High + Sex + corin + HSP70 + RBP4 + SERPINA12 + LowLevel |
| 296 | CAD ~ Intercept.High + Sex + corin + HSP70 + RBP4 + LowLevel |
| 297 | CAD ~ Intercept.High + Sex + corin + HSP70 + SERPINA12 + LowLevel |
| 298 | CAD ~ Intercept.High + Sex + corin + RBP4 + SERPINA12 + LowLevel |
| 299 | CAD ~ Intercept.High + Sex + HSP70 + RBP4 + SERPINA12 + LowLevel |
| 300 | CAD ~ Intercept.High + Age + APOB + corin + HSP70 + RBP4 + SERPINA12 + LowLevel |
| 301 | CAD ~ Intercept.High + Age + APOB + corin + HSP70 + RBP4 + SERPINA12 + LowLevel |
| 302 | CAD ~ Intercept.High + Age + APOB + corin + HSP70 + RBP4 + SERPINA12 + LowLevel |
| 303 | CAD ~ Intercept.High + Age + APOB + corin + HSP70 + RBP4 + SERPINA12 + LowLevel |
| 304 | CAD ~ Intercept.High + Age + APOB + corin + HSP70 + RBP4 + SERPINA12 + LowLevel |
| 305 | CAD ~ Intercept.High + Age + APOB + corin + HSP70 + RBP4 + SERPINA12 + LowLevel |
| 306 | CAD ~ Intercept.High + Age + APOB + corin + HSP70 + RBP4 + SERPINA12 + LowLevel |
| 307 | CAD ~ Intercept.High + Age + APOB + corin + HSP70 + RBP4 + SERPINA12 + LowLevel |
| 308 | CAD ~ Intercept.High + Age + APOB + corin + HSP70 + RBP4 + SERPINA12 + LowLevel |
| 309 | CAD ~ Intercept.High + Age + APOB + corin + HSP70 + RBP4 + SERPINA12 + LowLevel |
| 310 | CAD ~ Intercept.High + Age + APOB + corin + HSP70 + RBP4 + LowLevel |
| 311 | CAD ~ Intercept.High + Age + APOB + corin + HSP70 + RBP4 + LowLevel |
| 312 | CAD ~ Intercept.High + Age + APOB + corin + HSP70 + RBP4 + LowLevel |
| 313 | CAD ~ Intercept.High + Age + APOB + corin + HSP70 + RBP4 + LowLevel |
| 314 | CAD ~ Intercept.High + Age + APOB + corin + HSP70 + RBP4 + LowLevel |
| 315 | CAD ~ Intercept.High + Age + APOB + corin + HSP70 + RBP4 + LowLevel |
| 316 | CAD ~ Intercept.High + Age + APOB + corin + HSP70 + RBP4 + LowLevel |
| 317 | CAD ~ Intercept.High + Age + APOB + corin + HSP70 + RBP4 + LowLevel |
| 318 | CAD ~ Intercept.High + Age + APOB + corin + HSP70 + RBP4 + LowLevel |
| 319 | CAD ~ Intercept.High + Age + APOB + corin + HSP70 + RBP4 + LowLevel |
| 320 | CAD ~ Intercept.High + Age + APOB + corin + HSP70 + SERPINA12 + LowLevel |
| 321 | CAD ~ Intercept.High + Age + APOB + corin + HSP70 + SERPINA12 + LowLevel |
| 322 | CAD ~ Intercept.High + Age + APOB + corin + HSP70 + SERPINA12 + LowLevel |
| 323 | CAD ~ Intercept.High + Age + APOB + corin + HSP70 + SERPINA12 + LowLevel |
| 324 | CAD ~ Intercept.High + Age + APOB + corin + HSP70 + SERPINA12 + LowLevel |
| 325 | CAD ~ Intercept.High + Age + APOB + corin + HSP70 + SERPINA12 + LowLevel |
| 326 | CAD ~ Intercept.High + Age + APOB + corin + HSP70 + SERPINA12 + LowLevel |
| 327 | CAD ~ Intercept.High + Age + APOB + corin + HSP70 + SERPINA12 + LowLevel |
| 328 | CAD ~ Intercept.High + Age + APOB + corin + HSP70 + SERPINA12 + LowLevel |
| 329 | CAD ~ Intercept.High + Age + APOB + corin + HSP70 + SERPINA12 + LowLevel |
| 330 | CAD ~ Intercept.High + Age + APOB + corin + HSP70 + LowLevel |
| 331 | CAD ~ Intercept.High + Age + APOB + corin + HSP70 + LowLevel |
| 332 | CAD ~ Intercept.High + Age + APOB + corin + HSP70 + LowLevel |
| 333 | CAD ~ Intercept.High + Age + APOB + corin + HSP70 + LowLevel |
| 334 | CAD ~ Intercept.High + Age + APOB + corin + HSP70 + LowLevel |
| 335 | CAD ~ Intercept.High + Age + APOB + corin + RBP4 + SERPINA12 + LowLevel |
| 336 | CAD ~ Intercept.High + Age + APOB + corin + RBP4 + SERPINA12 + LowLevel |
| 337 | CAD ~ Intercept.High + Age + APOB + corin + RBP4 + SERPINA12 + LowLevel |
| 338 | CAD ~ Intercept.High + Age + APOB + corin + RBP4 + SERPINA12 + LowLevel |
| 339 | CAD ~ Intercept.High + Age + APOB + corin + RBP4 + SERPINA12 + LowLevel |
| 340 | CAD ~ Intercept.High + Age + APOB + corin + RBP4 + SERPINA12 + LowLevel |
| 341 | CAD ~ Intercept.High + Age + APOB + corin + RBP4 + SERPINA12 + LowLevel |
| 342 | CAD ~ Intercept.High + Age + APOB + corin + RBP4 + SERPINA12 + LowLevel |
| 343 | CAD ~ Intercept.High + Age + APOB + corin + RBP4 + SERPINA12 + LowLevel |
| 344 | CAD ~ Intercept.High + Age + APOB + corin + RBP4 + SERPINA12 + LowLevel |
| 345 | CAD ~ Intercept.High + Age + APOB + corin + RBP4 + LowLevel |
| 346 | CAD ~ Intercept.High + Age + APOB + corin + RBP4 + LowLevel |
| 347 | CAD ~ Intercept.High + Age + APOB + corin + RBP4 + LowLevel |
| 348 | CAD ~ Intercept.High + Age + APOB + corin + RBP4 + LowLevel |
| 349 | CAD ~ Intercept.High + Age + APOB + corin + RBP4 + LowLevel |
| 350 | CAD ~ Intercept.High + Age + APOB + corin + SERPINA12 + LowLevel |
| 351 | CAD ~ Intercept.High + Age + APOB + corin + SERPINA12 + LowLevel |
| 352 | CAD ~ Intercept.High + Age + APOB + corin + SERPINA12 + LowLevel |
| 353 | CAD ~ Intercept.High + Age + APOB + corin + SERPINA12 + LowLevel |
| 354 | CAD ~ Intercept.High + Age + APOB + corin + SERPINA12 + LowLevel |
| 355 | CAD ~ Intercept.High + Age + APOB + corin + LowLevel |
| 356 | CAD ~ Intercept.High + Age + APOB + HSP70 + RBP4 + SERPINA12 + LowLevel |
| 357 | CAD ~ Intercept.High + Age + APOB + HSP70 + RBP4 + SERPINA12 + LowLevel |
| 358 | CAD ~ Intercept.High + Age + APOB + HSP70 + RBP4 + SERPINA12 + LowLevel |
| 359 | CAD ~ Intercept.High + Age + APOB + HSP70 + RBP4 + SERPINA12 + LowLevel |
| 360 | CAD ~ Intercept.High + Age + APOB + HSP70 + RBP4 + SERPINA12 + LowLevel |
| 361 | CAD ~ Intercept.High + Age + APOB + HSP70 + RBP4 + SERPINA12 + LowLevel |
| 362 | CAD ~ Intercept.High + Age + APOB + HSP70 + RBP4 + SERPINA12 + LowLevel |
| 363 | CAD ~ Intercept.High + Age + APOB + HSP70 + RBP4 + SERPINA12 + LowLevel |
| 364 | CAD ~ Intercept.High + Age + APOB + HSP70 + RBP4 + SERPINA12 + LowLevel |
| 365 | CAD ~ Intercept.High + Age + APOB + HSP70 + RBP4 + SERPINA12 + LowLevel |
| 366 | CAD ~ Intercept.High + Age + APOB + HSP70 + RBP4 + LowLevel |
| 367 | CAD ~ Intercept.High + Age + APOB + HSP70 + RBP4 + LowLevel |
| 368 | CAD ~ Intercept.High + Age + APOB + HSP70 + RBP4 + LowLevel |
| 369 | CAD ~ Intercept.High + Age + APOB + HSP70 + RBP4 + LowLevel |
| 370 | CAD ~ Intercept.High + Age + APOB + HSP70 + RBP4 + LowLevel |
| 371 | CAD ~ Intercept.High + Age + APOB + HSP70 + SERPINA12 + LowLevel |
| 372 | CAD ~ Intercept.High + Age + APOB + HSP70 + SERPINA12 + LowLevel |
| 373 | CAD ~ Intercept.High + Age + APOB + HSP70 + SERPINA12 + LowLevel |
| 374 | CAD ~ Intercept.High + Age + APOB + HSP70 + SERPINA12 + LowLevel |
| 375 | CAD ~ Intercept.High + Age + APOB + HSP70 + SERPINA12 + LowLevel |
| 376 | CAD ~ Intercept.High + Age + APOB + HSP70 + LowLevel |
| 377 | CAD ~ Intercept.High + Age + APOB + RBP4 + SERPINA12 + LowLevel |
| 378 | CAD ~ Intercept.High + Age + APOB + RBP4 + SERPINA12 + LowLevel |
| 379 | CAD ~ Intercept.High + Age + APOB + RBP4 + SERPINA12 + LowLevel |
| 380 | CAD ~ Intercept.High + Age + APOB + RBP4 + SERPINA12 + LowLevel |
| 381 | CAD ~ Intercept.High + Age + APOB + RBP4 + SERPINA12 + LowLevel |
| 382 | CAD ~ Intercept.High + Age + APOB + RBP4 + LowLevel |
| 383 | CAD ~ Intercept.High + Age + APOB + SERPINA12 + LowLevel |
| 384 | CAD ~ Intercept.High + Age + corin + HSP70 + RBP4 + SERPINA12 + LowLevel |
| 385 | CAD ~ Intercept.High + Age + corin + HSP70 + RBP4 + SERPINA12 + LowLevel |
| 386 | CAD ~ Intercept.High + Age + corin + HSP70 + RBP4 + SERPINA12 + LowLevel |
| 387 | CAD ~ Intercept.High + Age + corin + HSP70 + RBP4 + SERPINA12 + LowLevel |
| 388 | CAD ~ Intercept.High + Age + corin + HSP70 + RBP4 + SERPINA12 + LowLevel |
| 389 | CAD ~ Intercept.High + Age + corin + HSP70 + RBP4 + SERPINA12 + LowLevel |
| 390 | CAD ~ Intercept.High + Age + corin + HSP70 + RBP4 + SERPINA12 + LowLevel |
| 391 | CAD ~ Intercept.High + Age + corin + HSP70 + RBP4 + SERPINA12 + LowLevel |
| 392 | CAD ~ Intercept.High + Age + corin + HSP70 + RBP4 + SERPINA12 + LowLevel |
| 393 | CAD ~ Intercept.High + Age + corin + HSP70 + RBP4 + SERPINA12 + LowLevel |
| 394 | CAD ~ Intercept.High + Age + corin + HSP70 + RBP4 + LowLevel |
| 395 | CAD ~ Intercept.High + Age + corin + HSP70 + RBP4 + LowLevel |
| 396 | CAD ~ Intercept.High + Age + corin + HSP70 + RBP4 + LowLevel |
| 397 | CAD ~ Intercept.High + Age + corin + HSP70 + RBP4 + LowLevel |
| 398 | CAD ~ Intercept.High + Age + corin + HSP70 + RBP4 + LowLevel |
| 399 | CAD ~ Intercept.High + Age + corin + HSP70 + SERPINA12 + LowLevel |
| 400 | CAD ~ Intercept.High + Age + corin + HSP70 + SERPINA12 + LowLevel |
| 401 | CAD ~ Intercept.High + Age + corin + HSP70 + SERPINA12 + LowLevel |
| 402 | CAD ~ Intercept.High + Age + corin + HSP70 + SERPINA12 + LowLevel |
| 403 | CAD ~ Intercept.High + Age + corin + HSP70 + SERPINA12 + LowLevel |
| 404 | CAD ~ Intercept.High + Age + corin + HSP70 + LowLevel |
| 405 | CAD ~ Intercept.High + Age + corin + RBP4 + SERPINA12 + LowLevel |
| 406 | CAD ~ Intercept.High + Age + corin + RBP4 + SERPINA12 + LowLevel |
| 407 | CAD ~ Intercept.High + Age + corin + RBP4 + SERPINA12 + LowLevel |
| 408 | CAD ~ Intercept.High + Age + corin + RBP4 + SERPINA12 + LowLevel |
| 409 | CAD ~ Intercept.High + Age + corin + RBP4 + SERPINA12 + LowLevel |
| 410 | CAD ~ Intercept.High + Age + corin + RBP4 + LowLevel |
| 411 | CAD ~ Intercept.High + Age + corin + SERPINA12 + LowLevel |
| 412 | CAD ~ Intercept.High + Age + HSP70 + RBP4 + SERPINA12 + LowLevel |
| 413 | CAD ~ Intercept.High + Age + HSP70 + RBP4 + SERPINA12 + LowLevel |
| 414 | CAD ~ Intercept.High + Age + HSP70 + RBP4 + SERPINA12 + LowLevel |
| 415 | CAD ~ Intercept.High + Age + HSP70 + RBP4 + SERPINA12 + LowLevel |
| 416 | CAD ~ Intercept.High + Age + HSP70 + RBP4 + SERPINA12 + LowLevel |
| 417 | CAD ~ Intercept.High + Age + HSP70 + RBP4 + LowLevel |
| 418 | CAD ~ Intercept.High + Age + HSP70 + SERPINA12 + LowLevel |
| 419 | CAD ~ Intercept.High + Age + RBP4 + SERPINA12 + LowLevel |
| 420 | CAD ~ Intercept.High + APOB + corin + HSP70 + RBP4 + SERPINA12 + LowLevel |
| 421 | CAD ~ Intercept.High + APOB + corin + HSP70 + RBP4 + SERPINA12 + LowLevel |
| 422 | CAD ~ Intercept.High + APOB + corin + HSP70 + RBP4 + SERPINA12 + LowLevel |
| 423 | CAD ~ Intercept.High + APOB + conn + HSP70 + RBP4 + SERPINA12 + LowLevel |
| 424 | CAD ~ Intercept.High + APOB + corin + HSP70 + RBP4 + SERPINA12 + LowLevel |
| 425 | CAD ~ Intercept.High + APOB + corin + HSP70 + RBP4 + SERPINA12 + LowLevel |
| 426 | CAD ~ Intercept.High + APOB + corin + HSP70 + RBP4 + SERPINA12 + LowLevel |
| 427 | CAD ~ Intercept.High + APOB + corin + HSP70 + RBP4 + SERPINA12 + LowLevel |
| 428 | CAD ~ Intercept.High + APOB + corin + HSP70 + RBP4 + SERPINA12 + LowLevel |
| 429 | CAD ~ Intercept.High + APOB + corin + HSP70 + RBP4 + SERPINA12 + LowLevel |
| 430 | CAD ~ Intercept.High + APOB + corin + HSP70 + RBP4 + LowLevel |
| 431 | CAD ~ Intercept.High + APOB + corin + HSP70 + RBP4 + LowLevel |
| 432 | CAD ~ Intercept.High + APOB + corin + HSP70 + RBP4 + LowLevel |
| 433 | CAD ~ Intercept.High + APOB + corin + HSP70 + RBP4 + LowLevel |
| 434 | CAD ~ Intercept.High + APOB + corin + HSP70 + RBP4 + LowLevel |
| 435 | CAD ~ Intercept.High + APOB + corin + HSP70 + SERPINA12 + LowLevel |
| 436 | CAD ~ Intercept.High + APOB + corin + HSP70 + SERPINA12 + LowLevel |
| 437 | CAD ~ Intercept.High + APOB + corin + HSP70 + SERPINA12 + LowLevel |
| 438 | CAD ~ Intercept.High + APOB + corin + HSP70 + SERPINA12 + LowLevel |
| 439 | CAD ~ Intercept.High + APOB + corin + HSP70 + SERPINA12 + LowLevel |
| 440 | CAD ~ Intercept.High + APOB + corin + HSP70 + LowLevel |
| 441 | CAD ~ Intercept.High + APOB + corin + RBP4 + SERPINA12 + LowLevel |
| 442 | CAD ~ Intercept.High + APOB + corin + RBP4 + SERPINA12 + LowLevel |
| 443 | CAD ~ Intercept.High + APOB + corin + RBP4 + SERPINA12 + LowLevel |
| 444 | CAD ~ Intercept.High + APOB + corin + RBP4 + SERPINA12 + LowLevel |
| 445 | CAD ~ Intercept.High + APOB + corin + RBP4 + SERPINA12 + LowLevel |
| 446 | CAD ~ Intercept.High + APOB + corin + RBP4 + LowLevel |
| 447 | CAD ~ Intercept.High + APOB + corin + SERPINA12 + LowLevel |
| 448 | CAD ~ Intercept.High + APOB + HSP70 + RBP4 + SERPINA12 + LowLevel |
| 449 | CAD ~ Intercept.High + APOB + HSP70 + RBP4 + SERPINA12 + LowLevel |
| 450 | CAD ~ Intercept.High + APOB + HSP70 + RBP4 + SERPINA12 + LowLevel |
| 451 | CAD ~ Intercept.High + APOB + HSP70 + RBP4 + SERPINA12 + LowLevel |
| 452 | CAD ~ Intercept.High + APOB + HSP70 + RBP4 + SERPINA12 + LowLevel |
| 453 | CAD ~ Intercept.High + APOB + HSP70 + RBP4 + LowLevel |
| 454 | CAD ~ Intercept.High + APOB + HSP70 + SERPINA12 + LowLevel |
| 455 | CAD ~ Intercept.High + APOB + RBP4 + SERPINA12 + LowLevel |
| 456 | CAD ~ Intercept.High + corin + HSP70 + RBP4 + SERPINA12 + LowLevel |
| 457 | CAD ~ Intercept.High + corin + HSP70 + RBP4 + SERPINA12 + LowLevel |
| 458 | CAD ~ Intercept.High + corin + HSP70 + RBP4 + SERPINA12 + LowLevel |
| 459 | CAD ~ Intercept.High + corin + HSP70 + RBP4 + SERPINA12 + LowLevel |
| 460 | CAD ~ Intercept.High + corin + HSP70 + RBP4 + SERPINA12 + LowLevel |
| 461 | CAD ~ Intercept.High + corin + HSP70 + RBP4 + LowLevel |
| 462 | CAD ~ Intercept.High + corin + HSP70 + SERPINA12 + LowLevel |
| 463 | CAD ~ Intercept.High + corin + RBP4 + SERPINA12 + LowLevel |
| 464 | CAD ~ Intercept.High + Sex + Age + APOB + corin + HSP70 + RBP4 + SERPINA12 + LowLevel |
| 465 | CAD ~ Intercept.High + Sex + Age + APOB + corin + HSP70 + RBP4 + SERPINA12 + LowLevel |
| 466 | CAD ~ Intercept.High + Sex + Age + APOB + corin + HSP70 + RBP4 + SERPINA12 + LowLevel |
| 467 | CAD ~ Intercept.High + Sex + Age + APOB + corin + HSP70 + RBP4 + SERPINA12 + LowLevel |
| 468 | CAD ~ Intercept.High + Sex + Age + APOB + corin + HSP70 + RBP4 + SERPINA12 + LowLevel |
| 469 | CAD ~ Intercept.High + Sex + Age + APOB + corin + HSP70 + RBP4 + LowLevel |
| 470 | CAD ~ Intercept.High + Sex + Age + APOB + corin + HSP70 + RBP4 + LowLevel |
| 471 | CAD ~ Intercept.High + Sex + Age + APOB + corin + HSP70 + RBP4 + LowLevel |
| 472 | CAD ~ Intercept.High + Sex + Age + APOB + corin + HSP70 + RBP4 + LowLevel |
| 473 | CAD ~ Intercept.High + Sex + Age + APOB + corin + HSP70 + RBP4 + LowLevel |
| 474 | CAD ~ Intercept.High + Sex + Age + APOB + corin + HSP70 + RBP4 + LowLevel |
| 475 | CAD ~ Intercept.High + Sex + Age + APOB + corin + HSP70 + RBP4 + LowLevel |
| 476 | CAD ~ Intercept.High + Sex + Age + APOB + corin + HSP70 + RBP4 + LowLevel |
| 477 | CAD ~ Intercept.High + Sex + Age + APOB + corin + HSP70 + RBP4 + LowLevel |
| 478 | CAD ~ Intercept.High + Sex + Age + APOB + corin + HSP70 + RBP4 + LowLevel |
| 479 | CAD ~ Intercept.High + Sex + Age + APOB + corin + HSP70 + SERPINA12 + LowLevel |
| 480 | CAD ~ Intercept.High + Sex + Age + APOB + corin + HSP70 + SERPINA12 + LowLevel |
| 481 | CAD ~ Intercept.High + Sex + Age + APOB + corin + HSP70 + SERPINA12 + LowLevel |
| 482 | CAD ~ Intercept.High + Sex + Age + APOB + corin + HSP70 + SERPINA12 + LowLevel |
| 483 | CAD ~ Intercept.High + Sex + Age + APOB + corin + HSP70 + SERPINA12 + LowLevel |
| 484 | CAD ~ Intercept.High + Sex + Age + APOB + corin + HSP70 + SERPINA12 + LowLevel |
| 485 | CAD ~ Intercept.High + Sex + Age + APOB + corin + HSP70 + SERPINA12 + LowLevel |
| 486 | CAD ~ Intercept.High + Sex + Age + APOB + corin + HSP70 + SERPINA12 + LowLevel |
| 487 | CAD ~ Intercept.High + Sex + Age + APOB + corin + HSP70 + SERPINA12 + LowLevel |
| 488 | CAD ~ Intercept.High + Sex + Age + APOB + corin + HSP70 + SERPINA12 + LowLevel |
| 489 | CAD ~ Intercept.High + Sex + Age + APOB + corin + HSP70 + LowLevel |
| 490 | CAD ~ Intercept.High + Sex + Age + APOB + corin + HSP70 + LowLevel |
| 491 | CAD ~ Intercept.High + Sex + Age + APOB + corin + HSP70 + LowLevel |
| 492 | CAD ~ Intercept.High + Sex + Age + APOB + corin + HSP70 + LowLevel |
| 493 | CAD ~ Intercept.High + Sex + Age + APOB + corin + HSP70 + LowLevel |
| 494 | CAD ~ Intercept.High + Sex + Age + APOB + corin + HSP70 + LowLevel |
| 495 | CAD ~ Intercept.High + Sex + Age + APOB + corin + HSP70 + LowLevel |
| 496 | CAD ~ Intercept.High + Sex + Age + APOB + corin + HSP70 + LowLevel |
| 497 | CAD ~ Intercept.High + Sex + Age + APOB + corin + HSP70 + LowLevel |
| 498 | CAD ~ Intercept.High + Sex + Age + APOB + corin + HSP70 + LowLevel |
| 499 | CAD ~ Intercept.High + Sex + Age + APOB + corin + RBP4 + SERPINA12 + LowLevel |
| 500 | CAD ~ Intercept.High + Sex + Age + APOB + corin + RBP4 + SERPINA12 + LowLevel |
| 501 | CAD ~ Intercept.High + Sex + Age + APOB + corin + RBP4 + SERPINA12 + LowLevel |
| 502 | CAD ~ Intercept.High + Sex + Age + APOB + corin + RBP4 + SERPINA12 + LowLevel |
| 503 | CAD ~ Intercept.High + Sex + Age + APOB + corin + RBP4 + SERPINA12 + LowLevel |
| 504 | CAD ~ Intercept.High + Sex + Age + APOB + corin + RBP4 + SERPINA12 + LowLevel |
| 505 | CAD ~ Intercept.High + Sex + Age + APOB + corin + RBP4 + SERPINA12 + LowLevel |
| 506 | CAD ~ Intercept.High + Sex + Age + APOB + corin + RBP4 + SERPINA12 + LowLevel |
| 507 | CAD ~ Intercept.High + Sex + Age + APOB + corin + RBP4 + SERPINA12 + LowLevel |
| 508 | CAD ~ Intercept.High + Sex + Age + APOB + corin + RBP4 + SERPINA12 + LowLevel |
| 509 | CAD ~ Intercept.High + Sex + Age + APOB + corin + RBP4 + LowLevel |
| 510 | CAD ~ Intercept.High + Sex + Age + APOB + corin + RBP4 + LowLevel |
| 511 | CAD ~ Intercept.High + Sex + Age + APOB + corin + RBP4 + LowLevel |
| 512 | CAD ~ Intercept.High + Sex + Age + APOB + corin + RBP4 + LowLevel |
| 513 | CAD ~ Intercept.High + Sex + Age + APOB + corin + RBP4 + LowLevel |
| 514 | CAD ~ Intercept.High + Sex + Age + APOB + corin + RBP4 + LowLevel |
| 515 | CAD ~ Intercept.High + Sex + Age + APOB + corin + RBP4 + LowLevel |
| 516 | CAD ~ Intercept.High + Sex + Age + APOB + corin + RBP4 + LowLevel |
| 517 | CAD ~ Intercept.High + Sex + Age + APOB + corin + RBP4 + LowLevel |
| 518 | CAD ~ Intercept.High + Sex + Age + APOB + corin + RBP4 + LowLevel |
| 519 | CAD ~ Intercept.High + Sex + Age + APOB + corin + SERPINA12 + LowLevel |
| 520 | CAD ~ Intercept.High + Sex + Age + APOB + corin + SERPINA12 + LowLevel |
| 521 | CAD ~ Intercept.High + Sex + Age + APOB + corin + SERPINA12 + LowLevel |
| 522 | CAD ~ Intercept.High + Sex + Age + APOB + corin + SERPINA12 + LowLevel |
| 523 | CAD ~ Intercept.High + Sex + Age + APOB + corin + SERPINA12 + LowLevel |
| 524 | CAD ~ Intercept.High + Sex + Age + APOB + corin + SERPINA12 + LowLevel |
| 525 | CAD ~ Intercept.High + Sex + Age + APOB + corin + SERPINA12 + LowLevel |
| 526 | CAD ~ Intercept.High + Sex + Age + APOB + corin + SERPINA12 + LowLevel |
| 527 | CAD ~ Intercept.High + Sex + Age + APOB + corin + SERPINA12 + LowLevel |
| 528 | CAD ~ Intercept.High + Sex + Age + APOB + corin + SERPINA12 + LowLevel |
| 529 | CAD ~ Intercept.High + Sex + Age + APOB + corin + LowLevel |
| 530 | CAD ~ Intercept.High + Sex + Age + APOB + corin + LowLevel |
| 531 | CAD ~ Intercept.High + Sex + Age + APOB + corin + LowLevel |
| 532 | CAD ~ Intercept.High + Sex + Age + APOB + corin + LowLevel |
| 533 | CAD ~ Intercept.High + Sex + Age + APOB + corin + LowLevel |
| 534 | CAD ~ Intercept.High + Sex + Age + APOB + HSP70 + RBP4 + SERPINA12 + LowLevel |
| 535 | CAD ~ Intercept.High + Sex + Age + APOB + HSP70 + RBP4 + SERPINA12 + LowLevel |
| 536 | CAD ~ Intercept.High + Sex + Age + APOB + HSP70 + RBP4 + SERPINA12 + LowLevel |
| 537 | CAD ~ Intercept.High + Sex + Age + APOB + HSP70 + RBP4 + SERPINA12 + LowLevel |
| 538 | CAD ~ Intercept.High + Sex + Age + APOB + HSP70 + RBP4 + SERPINA12 + LowLevel |
| 539 | CAD ~ Intercept.High + Sex + Age + APOB + HSP70 + RBP4 + SERPINA12 + LowLevel |
| 540 | CAD ~ Intercept.High + Sex + Age + APOB + HSP70 + RBP4 + SERPINA12 + LowLevel |
| 541 | CAD ~ Intercept.High + Sex + Age + APOB + HSP70 + RBP4 + SERPINA12 + LowLevel |
| 542 | CAD ~ Intercept.High + Sex + Age + APOB + HSP70 + RBP4 + SERPINA12 + LowLevel |
| 543 | CAD ~ Intercept.High + Sex + Age + APOB + HSP70 + RBP4 + SERPINA12 + LowLevel |
| 544 | CAD ~ Intercept.High + Sex + Age + APOB + HSP70 + RBP4 + LowLevel |
| 545 | CAD ~ Intercept.High + Sex + Age + APOB + HSP70 + RBP4 + LowLevel |
| 546 | CAD ~ Intercept.High + Sex + Age + APOB + HSP70 + RBP4 + LowLevel |
| 547 | CAD ~ Intercept.High + Sex + Age + APOB + HSP70 + RBP4 + LowLevel |
| 548 | CAD ~ Intercept.High + Sex + Age + APOB + HSP70 + RBP4 + LowLevel |
| 549 | CAD ~ Intercept.High + Sex + Age + APOB + HSP70 + RBP4 + LowLevel |
| 550 | CAD ~ Intercept.High + Sex + Age + APOB + HSP70 + RBP4 + LowLevel |
| 551 | CAD ~ Intercept.High + Sex + Age + APOB + HSP70 + RBP4 + LowLevel |
| 552 | CAD ~ Intercept.High + Sex + Age + APOB + HSP70 + RBP4 + LowLevel |
| 553 | CAD ~ Intercept.High + Sex + Age + APOB + HSP70 + RBP4 + LowLevel |
| 554 | CAD ~ Intercept.High + Sex + Age + APOB + HSP70 + SERPINA12 + LowLevel |
| 555 | CAD ~ Intercept.High + Sex + Age + APOB + HSP70 + SERPINA12 + LowLevel |
| 556 | CAD ~ Intercept.High + Sex + Age + APOB + HSP70 + SERPINA12 + LowLevel |
| 557 | CAD ~ Intercept.High + Sex + Age + APOB + HSP70 + SERPINA12 + LowLevel |
| 558 | CAD ~ Intercept.High + Sex + Age + APOB + HSP70 + SERPINA12 + LowLevel |
| 559 | CAD ~ Intercept.High + Sex + Age + APOB + HSP70 + SERPINA12 + LowLevel |
| 560 | CAD ~ Intercept.High + Sex + Age + APOB + HSP70 + SERPINA12 + LowLevel |
| 561 | CAD ~ Intercept.High + Sex + Age + APOB + HSP70 + SERPINA12 + LowLevel |
| 562 | CAD ~ Intercept.High + Sex + Age + APOB + HSP70 + SERPINA12 + LowLevel |
| 563 | CAD ~ Intercept.High + Sex + Age + APOB + HSP70 + SERPINA12 + LowLevel |
| 564 | CAD ~ Intercept.High + Sex + Age + APOB + HSP70 + LowLevel |
| 565 | CAD ~ Intercept.High + Sex + Age + APOB + HSP70 + LowLevel |
| 566 | CAD ~ Intercept.High + Sex + Age + APOB + HSP70 + LowLevel |
| 567 | CAD ~ Intercept.High + Sex + Age + APOB + HSP70 + LowLevel |
| 568 | CAD ~ Intercept.High + Sex + Age + APOB + HSP70 + LowLevel |
| 569 | CAD ~ Intercept.High + Sex + Age + APOB + RBP4 + SERPINA12 + LowLevel |
| 570 | CAD ~ Intercept.High + Sex + Age + APOB + RBP4 + SERPINA12 + LowLevel |
| 571 | CAD ~ Intercept.High + Sex + Age + APOB + RBP4 + SERPINA12 + LowLevel |
| 572 | CAD ~ Intercept.High + Sex + Age + APOB + RBP4 + SERPINA12 + LowLevel |
| 573 | CAD ~ Intercept.High + Sex + Age + APOB + RBP4 + SERPINA12 + LowLevel |
| 574 | CAD ~ Intercept.High + Sex + Age + APOB + RBP4 + SERPINA12 + LowLevel |
| 575 | CAD ~ Intercept.High + Sex + Age + APOB + RBP4 + SERPINA12 + LowLevel |
| 576 | CAD ~ Intercept.High + Sex + Age + APOB + RBP4 + SERPINA12 + LowLevel |
| 577 | CAD ~ Intercept.High + Sex + Age + APOB + RBP4 + SERPINA12 + LowLevel |
| 578 | CAD ~ Intercept.High + Sex + Age + APOB + RBP4 + SERPINA12 + LowLevel |
| 579 | CAD ~ Intercept.High + Sex + Age + APOB + RBP4 + LowLevel |
| 580 | CAD ~ Intercept.High + Sex + Age + APOB + RBP4 + LowLevel |
| 581 | CAD ~ Intercept.High + Sex + Age + APOB + RBP4 + LowLevel |
| 582 | CAD ~ Intercept.High + Sex + Age + APOB + RBP4 + LowLevel |
| 583 | CAD ~ Intercept.High + Sex + Age + APOB + RBP4 + LowLevel |
| 584 | CAD ~ Intercept.High + Sex + Age + APOB + SERPINA12 + LowLevel |
| 585 | CAD ~ Intercept.High + Sex + Age + APOB + SERPINA12 + LowLevel |
| 586 | CAD ~ Intercept.High + Sex + Age + APOB + SERPINA12 + LowLevel |
| 587 | CAD ~ Intercept.High + Sex + Age + APOB + SERPINA12 + LowLevel |
| 588 | CAD ~ Intercept.High + Sex + Age + APOB + SERPINA12 + LowLevel |
| 589 | CAD ~ Intercept.High + Sex + Age + APOB + LowLevel |
| 590 | CAD ~ Intercept.High + Sex + Age + corin + HSP70 + RBP4 + SERPINA12 + LowLevel |
| 591 | CAD ~ Intercept.High + Sex + Age + corin + HSP70 + RBP4 + SERPINA12 + LowLevel |
| 592 | CAD ~ Intercept.High + Sex + Age + corin + HSP70 + RBP4 + SERPINA12 + LowLevel |
| 593 | CAD ~ Intercept.High + Sex + Age + corin + HSP70 + RBP4 + SERPINA12 + LowLevel |
| 594 | CAD ~ Intercept.High + Sex + Age + corin + HSP70 + RBP4 + SERPINA12 + LowLevel |
| 595 | CAD ~ Intercept.High + Sex + Age + corin + HSP70 + RBP4 + SERPINA12 + LowLevel |
| 596 | CAD ~ Intercept.High + Sex + Age + corin + HSP70 + RBP4 + SERPINA12 + LowLevel |
| 597 | CAD ~ Intercept.High + Sex + Age + corin + HSP70 + RBP4 + SERPINA12 + LowLevel |
| 598 | CAD ~ Intercept.High + Sex + Age + corin + HSP70 + RBP4 + SERPINA12 + LowLevel |
| 599 | CAD ~ Intercept.High + Sex + Age + corin + HSP70 + RBP4 + SERPINA12 + LowLevel |
| 600 | CAD ~ Intercept.High + Sex + Age + corin + HSP70 + RBP4 + LowLevel |
| 601 | CAD ~ Intercept.High + Sex + Age + corin + HSP70 + RBP4 + LowLevel |
| 602 | CAD ~ Intercept.High + Sex + Age + corin + HSP70 + RBP4 + LowLevel |
| 603 | CAD ~ Intercept.High + Sex + Age + corin + HSP70 + RBP4 + LowLevel |
| 604 | CAD ~ Intercept.High + Sex + Age + corin + HSP70 + RBP4 + LowLevel |
| 605 | CAD ~ Intercept.High + Sex + Age + corin + HSP70 + RBP4 + LowLevel |
| 606 | CAD ~ Intercept.High + Sex + Age + corin + HSP70 + RBP4 + LowLevel |
| 607 | CAD ~ Intercept.High + Sex + Age + corin + HSP70 + RBP4 + LowLevel |
| 608 | CAD ~ Intercept.High + Sex + Age + corin + HSP70 + RBP4 + LowLevel |
| 609 | CAD ~ Intercept.High + Sex + Age + corin + HSP70 + RBP4 + LowLevel |
| 610 | CAD ~ Intercept.High + Sex + Age + corin + HSP70 + SERPINA12 + LowLevel |
| 611 | CAD ~ Intercept.High + Sex + Age + corin + HSP70 + SERPINA12 + LowLevel |
| 612 | CAD ~ Intercept.High + Sex + Age + corin + HSP70 + SERPINA12 + LowLevel |
| 613 | CAD ~ Intercept.High + Sex + Age + corin + HSP70 + SERPINA12 + LowLevel |
| 614 | CAD ~ Intercept.High + Sex + Age + corin + HSP70 + SERPINA12 + LowLevel |
| 615 | CAD ~ Intercept.High + Sex + Age + corin + HSP70 + SERPINA12 + LowLevel |
| 616 | CAD ~ Intercept.High + Sex + Age + corin + HSP70 + SERPINA12 + LowLevel |
| 617 | CAD ~ Intercept.High + Sex + Age + corin + HSP70 + SERPINA12 + LowLevel |
| 618 | CAD ~ Intercept.High + Sex + Age + corin + HSP70 + SERPINA12 + LowLevel |
| 619 | CAD ~ Intercept.High + Sex + Age + corin + HSP70 + SERPINA12 + LowLevel |
| 620 | CAD ~ Intercept.High + Sex + Age + corin + HSP70 + LowLevel |
| 621 | CAD ~ Intercept.High + Sex + Age + corin + HSP70 + LowLevel |
| 622 | CAD ~ Intercept.High + Sex + Age + corin + HSP70 + LowLevel |
| 623 | CAD ~ Intercept.High + Sex + Age + corin + HSP70 + LowLevel |
| 624 | CAD ~ Intercept.High + Sex + Age + corin + HSP70 + LowLevel |
| 625 | CAD ~ Intercept.High + Sex + Age + corin + RBP4 + SERPINA12 + LowLevel |
| 626 | CAD ~ Intercept.High + Sex + Age + corin + RBP4 + SERPINA12 + LowLevel |
| 627 | CAD ~ Intercept.High + Sex + Age + corin + RBP4 + SERPINA12 + LowLevel |
| 628 | CAD ~ Intercept.High + Sex + Age + corin + RBP4 + SERPINA12 + LowLevel |
| 629 | CAD ~ Intercept.High + Sex + Age + corin + RBP4 + SERPINA12 + LowLevel |
| 630 | CAD ~ Intercept.High + Sex + Age + corin + RBP4 + SERPINA12 + LowLevel |
| 631 | CAD ~ Intercept.High + Sex + Age + corin + RBP4 + SERPINA12 + LowLevel |
| 632 | CAD ~ Intercept.High + Sex + Age + corin + RBP4 + SERPINA12 + LowLevel |
| 633 | CAD ~ Intercept.High + Sex + Age + corin + RBP4 + SERPINA12 + LowLevel |
| 634 | CAD ~ Intercept.High + Sex + Age + corin + RBP4 + SERPINA12 + LowLevel |
| 635 | CAD ~ Intercept.High + Sex + Age + corin + RBP4 + LowLevel |
| 636 | CAD ~ Intercept.High + Sex + Age + corin + RBP4 + LowLevel |
| 637 | CAD ~ Intercept.High + Sex + Age + corin + RBP4 + LowLevel |
| 638 | CAD ~ Intercept.High + Sex + Age + corin + RBP4 + LowLevel |
| 639 | CAD ~ Intercept.High + Sex + Age + corin + RBP4 + LowLevel |
| 640 | CAD ~ Intercept.High + Sex + Age + corin + SERPINA12 + LowLevel |
| 641 | CAD ~ Intercept.High + Sex + Age + corin + SERPINA12 + LowLevel |
| 642 | CAD ~ Intercept.High + Sex + Age + corin + SERPINA12 + LowLevel |
| 643 | CAD ~ Intercept.High + Sex + Age + corin + SERPINA12 + LowLevel |
| 644 | CAD ~ Intercept.High + Sex + Age + corin + SERPINA12 + LowLevel |
| 645 | CAD ~ Intercept.High + Sex + Age + corin + LowLevel |
| 646 | CAD ~ Intercept.High + Sex + Age + HSP70 + RBP4 + SERPINA12 + LowLevel |
| 647 | CAD ~ Intercept.High + Sex + Age + HSP70 + RBP4 + SERPINA12 + LowLevel |
| 648 | CAD ~ Intercept.High + Sex + Age + HSP70 + RBP4 + SERPINA12 + LowLevel |
| 649 | CAD ~ Intercept.High + Sex + Age + HSP70 + RBP4 + SERPINA12 + LowLevel |
| 650 | CAD ~ Intercept.High + Sex + Age + HSP70 + RBP4 + SERPINA12 + LowLevel |
| 651 | CAD ~ Intercept.High + Sex + Age + HSP70 + RBP4 + SERPINA12 + LowLevel |
| 652 | CAD ~ Intercept.High + Sex + Age + HSP70 + RBP4 + SERPINA12 + LowLevel |
| 653 | CAD ~ Intercept.High + Sex + Age + HSP70 + RBP4 + SERPINA12 + LowLevel |
| 654 | CAD ~ Intercept.High + Sex + Age + HSP70 + RBP4 + SERPINA12 + LowLevel |
| 655 | CAD ~ Intercept.High + Sex + Age + HSP70 + RBP4 + SERPINA12 + LowLevel |
| 656 | CAD ~ Intercept.High + Sex + Age + HSP70 + RBP4 + LowLevel |
| 657 | CAD ~ Intercept.High + Sex + Age + HSP70 + RBP4 + LowLevel |
| 658 | CAD ~ Intercept.High + Sex + Age + HSP70 + RBP4 + LowLevel |
| 659 | CAD ~ Intercept.High + Sex + Age + HSP70 + RBP4 + LowLevel |
| 660 | CAD ~ Intercept.High + Sex + Age + HSP70 + RBP4 + LowLevel |
| 661 | CAD ~ Intercept.High + Sex + Age + HSP70 + SERPINA12 + LowLevel |
| 662 | CAD ~ Intercept.High + Sex + Age + HSP70 + SERPINA12 + LowLevel |
| 663 | CAD ~ Intercept.High + Sex + Age + HSP70 + SERPINA12 + LowLevel |
| 664 | CAD ~ Intercept.High + Sex + Age + HSP70 + SERPINA12 + LowLevel |
| 665 | CAD ~ Intercept.High + Sex + Age + HSP70 + SERPINA12 + LowLevel |
| 666 | CAD ~ Intercept.High + Sex + Age + HSP70 + LowLevel |
| 667 | CAD ~ Intercept.High + Sex + Age + RBP4 + SERPINA12 + LowLevel |
| 668 | CAD ~ Intercept.High + Sex + Age + RBP4 + SERPINA12 + LowLevel |
| 669 | CAD ~ Intercept.High + Sex + Age + RBP4 + SERPINA12 + LowLevel |
| 670 | CAD ~ Intercept.High + Sex + Age + RBP4 + SERPINA12 + LowLevel |
| 671 | CAD ~ Intercept.High + Sex + Age + RBP4 + SERPINA12 + LowLevel |
| 672 | CAD ~ Intercept.High + Sex + Age + RBP4 + LowLevel |
| 673 | CAD ~ Intercept.High + Sex + Age + SERPINA12 + LowLevel |
| 674 | CAD ~ Intercept.High + Sex + APOB + corin + HSP70 + RBP4 + SERPINA12 + LowLevel |
| 675 | CAD ~ Intercept.High + Sex + APOB + corin + HSP70 + RBP4 + SERPINA12 + LowLevel |
| 676 | CAD ~ Intercept.High + Sex + APOB + corin + HSP70 + RBP4 + SERPINA12 + LowLevel |
| 677 | CAD ~ Intercept.High + Sex + APOB + corin + HSP70 + RBP4 + SERPINA12 + LowLevel |
| 678 | CAD ~ Intercept.High + Sex + APOB + corin + HSP70 + RBP4 + SERPINA12 + LowLevel |
| 679 | CAD ~ Intercept.High + Sex + APOB + corin + HSP70 + RBP4 + SERPINA12 + LowLevel |
| 680 | CAD ~ Intercept.High + Sex + APOB + corin + HSP70 + RBP4 + SERPINA12 + LowLevel |
| 681 | CAD ~ Intercept.High + Sex + APOB + corin + HSP70 + RBP4 + SERPINA12 + LowLevel |
| 682 | CAD ~ Intercept.High + Sex + APOB + corin + HSP70 + RBP4 + SERPINA12 + LowLevel |
| 683 | CAD ~ Intercept.High + Sex + APOB + corin + HSP70 + RBP4 + SERPINA12 + LowLevel |
| 684 | CAD ~ Intercept.High + Sex + APOB + corin + HSP70 + RBP4 + LowLevel |
| 685 | CAD ~ Intercept.High + Sex + APOB + corin + HSP70 + RBP4 + LowLevel |
| 686 | CAD ~ Intercept.High + Sex + APOB + corin + HSP70 + RBP4 + LowLevel |
| 687 | CAD ~ Intercept.High + Sex + APOB + corin + HSP70 + RBP4 + LowLevel |
| 688 | CAD ~ Intercept.High + Sex + APOB + corin + HSP70 + RBP4 + LowLevel |
| 689 | CAD ~ Intercept.High + Sex + APOB + corin + HSP70 + RBP4 + LowLevel |
| 690 | CAD ~ Intercept.High + Sex + APOB + corin + HSP70 + RBP4 + LowLevel |
| 691 | CAD ~ Intercept.High + Sex + APOB + corin + HSP70 + RBP4 + LowLevel |
| 692 | CAD ~ Intercept.High + Sex + APOB + corin + HSP70 + RBP4 + LowLevel |
| 693 | CAD ~ Intercept.High + Sex + APOB + corin + HSP70 + RBP4 + LowLevel |
| 694 | CAD ~ Intercept.High + Sex + APOB + corin + HSP70 + SERPINA12 + LowLevel |
| 695 | CAD ~ Intercept.High + Sex + APOB + corin + HSP70 + SERPINA12 + LowLevel |
| 696 | CAD ~ Intercept.High + Sex + APOB + corin + HSP70 + SERPINA12 + LowLevel |
| 697 | CAD ~ Intercept.High + Sex + APOB + corin + HSP70 + SERPINA12 + LowLevel |
| 698 | CAD ~ Intercept.High + Sex + APOB + corin + HSP70 + SERPINA12 + LowLevel |
| 699 | CAD ~ Intercept.High + Sex + APOB + corin + HSP70 + SERPINA12 + LowLevel |
| 700 | CAD ~ Intercept.High + Sex + APOB + corin + HSP70 + SERPINA12 + LowLevel |
| 701 | CAD ~ Intercept.High + Sex + APOB + corin + HSP70 + SERPINA12 + LowLevel |
| 702 | CAD ~ Intercept.High + Sex + APOB + corin + HSP70 + SERPINA12 + LowLevel |
| 703 | CAD ~ Intercept.High + Sex + APOB + corin + HSP70 + SERPINA12 + LowLevel |
| 704 | CAD ~ Intercept.High + Sex + APOB + corin + HSP70 + LowLevel |
| 705 | CAD ~ Intercept.High + Sex + APOB + corin + HSP70 + LowLevel |
| 706 | CAD ~ Intercept.High + Sex + APOB + corin + HSP70 + LowLevel |
| 707 | CAD ~ Intercept.High + Sex + APOB + corin + HSP70 + LowLevel |
| 708 | CAD ~ Intercept.High + Sex + APOB + corin + HSP70 + LowLevel |
| 709 | CAD ~ Intercept.High + Sex + APOB + corin + RBP4 + SERPINA12 + LowLevel |
| 710 | CAD ~ Intercept.High + Sex + APOB + corin + RBP4 + SERPINA12 + LowLevel |
| 711 | CAD ~ Intercept.High + Sex + APOB + corin + RBP4 + SERPINA12 + LowLevel |
| 712 | CAD ~ Intercept.High + Sex + APOB + corin + RBP4 + SERPINA12 + LowLevel |
| 713 | CAD ~ Intercept.High + Sex + APOB + corin + RBP4 + SERPINA12 + LowLevel |
| 714 | CAD ~ Intercept.High + Sex + APOB + corin + RBP4 + SERPINA12 + LowLevel |
| 715 | CAD ~ Intercept.High + Sex + APOB + corin + RBP4 + SERPINA12 + LowLevel |
| 716 | CAD ~ Intercept.High + Sex + APOB + corin + RBP4 + SERPINA12 + LowLevel |
| 717 | CAD ~ Intercept.High + Sex + APOB + corin + RBP4 + SERPINA12 + LowLevel |
| 718 | CAD ~ Intercept.High + Sex + APOB + corin + RBP4 + SERPINA12 + LowLevel |
| 719 | CAD ~ Intercept.High + Sex + APOB + corin + RBP4 + LowLevel |
| 720 | CAD ~ Intercept.High + Sex + APOB + corin + RBP4 + LowLevel |
| 721 | CAD ~ Intercept.High + Sex + APOB + corin + RBP4 + LowLevel |
| 722 | CAD ~ Intercept.High + Sex + APOB + corin + RBP4 + LowLevel |
| 723 | CAD ~ Intercept.High + Sex + APOB + corin + RBP4 + LowLevel |
| 724 | CAD ~ Intercept.High + Sex + APOB + corin + SERPINA12 + LowLevel |
| 725 | CAD ~ Intercept.High + Sex + APOB + corin + SERPINA12 + LowLevel |
| 726 | CAD ~ Intercept.High + Sex + APOB + corin + SERPINA12 + LowLevel |
| 727 | CAD ~ Intercept.High + Sex + APOB + corin + SERPINA12 + LowLevel |
| 728 | CAD ~ Intercept.High + Sex + APOB + corin + SERPINA12 + LowLevel |
| 729 | CAD ~ Intercept.High + Sex + APOB + corin + LowLevel |
| 730 | CAD ~ Intercept.High + Sex + APOB + HSP70 + RBP4 + SERPINA12 + LowLevel |
| 731 | CAD ~ Intercept.High + Sex + APOB + HSP70 + RBP4 + SERPINA12 + LowLevel |
| 732 | CAD ~ Intercept.High + Sex + APOB + HSP70 + RBP4 + SERPINA12 + LowLevel |
| 733 | CAD ~ Intercept.High + Sex + APOB + HSP70 + RBP4 + SERPINA12 + LowLevel |
| 734 | CAD ~ Intercept.High + Sex + APOB + HSP70 + RBP4 + SERPINA12 + LowLevel |
| 735 | CAD ~ Intercept.High + Sex + APOB + HSP70 + RBP4 + SERPINA12 + LowLevel |
| 736 | CAD ~ Intercept.High + Sex + APOB + HSP70 + RBP4 + SERPINA12 + LowLevel |
| 737 | CAD ~ Intercept.High + Sex + APOB + HSP70 + RBP4 + SERPINA12 + LowLevel |
| 738 | CAD ~ Intercept.High + Sex + APOB + HSP70 + RBP4 + SERPINA12 + LowLevel |
| 739 | CAD ~ Intercept.High + Sex + APOB + HSP70 + RBP4 + SERPINA12 + LowLevel |
| 740 | CAD ~ Intercept.High + Sex + APOB + HSP70 + RBP4 + LowLevel |
| 741 | CAD ~ Intercept.High + Sex + APOB + HSP70 + RBP4 + LowLevel |
| 742 | CAD ~ Intercept.High + Sex + APOB + HSP70 + RBP4 + LowLevel |
| 743 | CAD ~ Intercept.High + Sex + APOB + HSP70 + RBP4 + LowLevel |
| 744 | CAD ~ Intercept.High + Sex + APOB + HSP70 + RBP4 + LowLevel |
| 745 | CAD ~ Intercept.High + Sex + APOB + HSP70 + SERPINA12 + LowLevel |
| 746 | CAD ~ Intercept.High + Sex + APOB + HSP70 + SERPINA12 + LowLevel |
| 747 | CAD ~ Intercept.High + Sex + APOB + HSP70 + SERPINA12 + LowLevel |
| 748 | CAD ~ Intercept.High + Sex + APOB + HSP70 + SERPINA12 + LowLevel |
| 749 | CAD ~ Intercept.High + Sex + APOB + HSP70 + SERPINA12 + LowLevel |
| 750 | CAD ~ Intercept.High + Sex + APOB + HSP70 + LowLevel |
| 751 | CAD ~ Intercept.High + Sex + APOB + RBP4 + SERPINA12 + LowLevel |
| 752 | CAD ~ Intercept.High + Sex + APOB + RBP4 + SERPINA12 + LowLevel |
| 753 | CAD ~ Intercept.High + Sex + APOB + RBP4 + SERPINA12 + LowLevel |
| 754 | CAD ~ Intercept.High + Sex + APOB + RBP4 + SERPINA12 + LowLevel |
| 755 | CAD ~ Intercept.High + Sex + APOB + RBP4 + SERPINA12 + LowLevel |
| 756 | CAD ~ Intercept.High + Sex + APOB + RBP4 + LowLevel |
| 757 | CAD ~ Intercept.High + Sex + APOB + SERPINA12 + LowLevel |
| 758 | CAD ~ Intercept.High + Sex + corin + HSP70 + RBP4 + SERPINA12 + LowLevel |
| 759 | CAD ~ Intercept.High + Sex + corin + HSP70 + RBP4 + SERPINA12 + LowLevel |
| 760 | CAD ~ Intercept.High + Sex + corin + HSP70 + RBP4 + SERPINA12 + LowLevel |
| 761 | CAD ~ Intercept.High + Sex + corin + HSP70 + RBP4 + SERPINA12 + LowLevel |
| 762 | CAD ~ Intercept.High + Sex + corin + HSP70 + RBP4 + SERPINA12 + LowLevel |
| 763 | CAD ~ Intercept.High + Sex + corin + HSP70 + RBP4 + SERPINA12 + LowLevel |
| 764 | CAD ~ Intercept.High + Sex + corin + HSP70 + RBP4 + SERPINA12 + LowLevel |
| 765 | CAD ~ Intercept.High + Sex + corin + HSP70 + RBP4 + SERPINA12 + LowLevel |
| 766 | CAD ~ Intercept.High + Sex + corin + HSP70 + RBP4 + SERPINA12 + LowLevel |
| 767 | CAD ~ Intercept.High + Sex + corin + HSP70 + RBP4 + SERPINA12 + LowLevel |
| 768 | CAD ~ Intercept.High + Sex + corin + HSP70 + RBP4 + LowLevel |
| 769 | CAD ~ Intercept.High + Sex + corin + HSP70 + RBP4 + LowLevel |
| 770 | CAD ~ Intercept.High + Sex + corin + HSP70 + RBP4 + LowLevel |
| 771 | CAD ~ Intercept.High + Sex + corin + HSP70 + RBP4 + LowLevel |
| 772 | CAD ~ Intercept.High + Sex + corin + HSP70 + RBP4 + LowLevel |
| 773 | CAD ~ Intercept.High + Sex + corin + HSP70 + SERPINA12 + LowLevel |
| 774 | CAD ~ Intercept.High + Sex + corin + HSP70 + SERPINA12 + LowLevel |
| 775 | CAD ~ Intercept.High + Sex + corin + HSP70 + SERPINA12 + LowLevel |
| 776 | CAD ~ Intercept.High + Sex + corin + HSP70 + SERPINA12 + LowLevel |
| 777 | CAD ~ Intercept.High + Sex + corin + HSP70 + SERPINA12 + LowLevel |
| 778 | CAD ~ Intercept.High + Sex + corin + HSP70 + LowLevel |
| 779 | CAD ~ Intercept.High + Sex + corin + RBP4 + SERPINA12 + LowLevel |
| 780 | CAD ~ Intercept.High + Sex + corin + RBP4 + SERPINA12 + LowLevel |
| 781 | CAD ~ Intercept.High + Sex + corin + RBP4 + SERPINA12 + LowLevel |
| 782 | CAD ~ Intercept.High + Sex + corin + RBP4 + SERPINA12 + LowLevel |
| 783 | CAD ~ Intercept.High + Sex + corin + RBP4 + SERPINA12 + LowLevel |
| 784 | CAD ~ Intercept.High + Sex + corin + RBP4 + LowLevel |
| 785 | CAD ~ Intercept.High + Sex + corin + SERPINA12 + LowLevel |
| 786 | CAD ~ Intercept.High + Sex + HSP70 + RBP4 + SERPINA12 + LowLevel |
| 787 | CAD ~ Intercept.High + Sex + HSP70 + RBP4 + SERPINA12 + LowLevel |
| 788 | CAD ~ Intercept.High + Sex + HSP70 + RBP4 + SERPINA12 + LowLevel |
| 789 | CAD ~ Intercept.High + Sex + HSP70 + RBP4 + SERPINA12 + LowLevel |
| 790 | CAD ~ Intercept.High + Sex + HSP70 + RBP4 + SERPINA12 + LowLevel |
| 791 | CAD ~ Intercept.High + Sex + HSP70 + RBP4 + LowLevel |
| 792 | CAD ~ Intercept.High + Sex + HSP70 + SERPINA12 + LowLevel |
| 793 | CAD ~ Intercept.High + Sex + RBP4 + SERPINA12 + LowLevel |
| 794 | CAD ~ Intercept.High + HSP70 + RBP4 + SERPINA12 + LowLevel |
| 795 | CAD ~ Intercept.High + Age + APOB + corin + HSP70 + RBP4 + SERPINA12 + LowLevel |
| 796 | CAD ~ Intercept.High + Age + APOB + corin + HSP70 + RBP4 + SERPINA12 + LowLevel |
| 797 | CAD ~ Intercept.High + Age + APOB + corin + HSP70 + RBP4 + SERPINA12 + LowLevel |
| 798 | CAD ~ Intercept.High + Age + APOB + corin + HSP70 + RBP4 + SERPINA12 + LowLevel |
| 799 | CAD ~ Intercept.High + Age + APOB + corin + HSP70 + RBP4 + SERPINA12 + LowLevel |
| 800 | CAD ~ Intercept.High + Age + APOB + corin + HSP70 + RBP4 + LowLevel |
| 801 | CAD ~ Intercept.High + Age + APOB + corin + HSP70 + RBP4 + LowLevel |
| 802 | CAD ~ Intercept.High + Age + APOB + corin + HSP70 + RBP4 + LowLevel |
| 803 | CAD ~ Intercept.High + Age + APOB + corin + HSP70 + RBP4 + LowLevel |
| 804 | CAD ~ Intercept.High + Age + APOB + corin + HSP70 + RBP4 + LowLevel |
| 805 | CAD ~ Intercept.High + Age + APOB + corin + HSP70 + RBP4 + LowLevel |
| 806 | CAD ~ Intercept.High + Age + APOB + corin + HSP70 + RBP4 + LowLevel |
| 807 | CAD ~ Intercept.High + Age + APOB + corin + HSP70 + RBP4 + LowLevel |
| 808 | CAD ~ Intercept.High + Age + APOB + corin + HSP70 + RBP4 + LowLevel |
| 809 | CAD ~ Intercept.High + Age + APOB + corin + HSP70 + RBP4 + LowLevel |
| 810 | CAD ~ Intercept.High + Age + APOB + corin + HSP70 + SERPINA12 + LowLevel |
| 811 | CAD ~ Intercept.High + Age + APOB + corin + HSP70 + SERPINA12 + LowLevel |
| 812 | CAD ~ Intercept.High + Age + APOB + corin + HSP70 + SERPINA12 + LowLevel |
| 813 | CAD ~ Intercept.High + Age + APOB + corin + HSP70 + SERPINA12 + LowLevel |
| 814 | CAD ~ Intercept.High + Age + APOB + corin + HSP70 + SERPINA12 + LowLevel |
| 815 | CAD ~ Intercept.High + Age + APOB + corin + HSP70 + SERPINA12 + LowLevel |
| 816 | CAD ~ Intercept.High + Age + APOB + corin + HSP70 + SERPINA12 + LowLevel |
| 817 | CAD ~ Intercept.High + Age + APOB + corin + HSP70 + SERPINA12 + LowLevel |
| 818 | CAD ~ Intercept.High + Age + APOB + corin + HSP70 + SERPINA12 + LowLevel |
| 819 | CAD ~ Intercept.High + Age + APOB + corin + HSP70 + SERPINA12 + LowLevel |
| 820 | CAD ~ Intercept.High + Age + APOB + corin + HSP70 + LowLevel |
| 821 | CAD ~ Intercept.High + Age + APOB + corin + HSP70 + LowLevel |
| 822 | CAD ~ Intercept.High + Age + APOB + corin + HSP70 + LowLevel |
| 823 | CAD ~ Intercept.High + Age + APOB + corin + HSP70 + LowLevel |
| 824 | CAD ~ Intercept.High + Age + APOB + corin + HSP70 + LowLevel |
| 825 | CAD ~ Intercept.High + Age + APOB + corin + HSP70 + LowLevel |
| 826 | CAD ~ Intercept.High + Age + APOB + corin + HSP70 + LowLevel |
| 827 | CAD ~ Intercept.High + Age + APOB + corin + HSP70 + LowLevel |
| 828 | CAD ~ Intercept.High + Age + APOB + corin + HSP70 + LowLevel |
| 829 | CAD ~ Intercept.High + Age + APOB + corin + HSP70 + LowLevel |
| 830 | CAD ~ Intercept.High + Age + APOB + corin + RBP4 + SERPINA12 + LowLevel |
| 831 | CAD ~ Intercept.High + Age + APOB + corin + RBP4 + SERPINA12 + LowLevel |
| 832 | CAD ~ Intercept.High + Age + APOB + corin + RBP4 + SERPINA12 + LowLevel |
| 833 | CAD ~ Intercept.High + Age + APOB + corin + RBP4 + SERPINA12 + LowLevel |
| 834 | CAD ~ Intercept.High + Age + APOB + corin + RBP4 + SERPINA12 + LowLevel |
| 835 | CAD ~ Intercept.High + Age + APOB + corin + RBP4 + SERPINA12 + LowLevel |
| 836 | CAD ~ Intercept.High + Age + APOB + corin + RBP4 + SERPINA12 + LowLevel |
| 837 | CAD ~ Intercept.High + Age + APOB + corin + RBP4 + SERPINA12 + LowLevel |
| 838 | CAD ~ Intercept.High + Age + APOB + corin + RBP4 + SERPINA12 + LowLevel |
| 839 | CAD ~ Intercept.High + Age + APOB + corin + RBP4 + SERPINA12 + LowLevel |
| 840 | CAD ~ Intercept.High + Age + APOB + corin + RBP4 + LowLevel |
| 841 | CAD ~ Intercept.High + Age + APOB + corin + RBP4 + LowLevel |
| 842 | CAD ~ Intercept.High + Age + APOB + corin + RBP4 + LowLevel |
| 843 | CAD ~ Intercept.High + Age + APOB + corin + RBP4 + LowLevel |
| 844 | CAD ~ Intercept.High + Age + APOB + corin + RBP4 + LowLevel |
| 845 | CAD ~ Intercept.High + Age + APOB + corin + RBP4 + LowLevel |
| 846 | CAD ~ Intercept.High + Age + APOB + corin + RBP4 + LowLevel |
| 847 | CAD ~ Intercept.High + Age + APOB + corin + RBP4 + LowLevel |
| 848 | CAD ~ Intercept.High + Age + APOB + corin + RBP4 + LowLevel |
| 849 | CAD ~ Intercept.High + Age + APOB + corin + RBP4 + LowLevel |
| 850 | CAD ~ Intercept.High + Age + APOB + corin + SERPINA12 + LowLevel |
| 851 | CAD ~ Intercept.High + Age + APOB + corin + SERPINA12 + LowLevel |
| 852 | CAD ~ Intercept.High + Age + APOB + corin + SERPINA12 + LowLevel |
| 853 | CAD ~ Intercept.High + Age + APOB + corin + SERPINA12 + LowLevel |
| 854 | CAD ~ Intercept.High + Age + APOB + corin + SERPINA12 + LowLevel |
| 855 | CAD ~ Intercept.High + Age + APOB + corin + SERPINA12 + LowLevel |
| 856 | CAD ~ Intercept.High + Age + APOB + corin + SERPINA12 + LowLevel |
| 857 | CAD ~ Intercept.High + Age + APOB + corin + SERPINA12 + LowLevel |
| 858 | CAD ~ Intercept.High + Age + APOB + corin + SERPINA12 + LowLevel |
| 859 | CAD ~ Intercept.High + Age + APOB + corin + SERPINA12 + LowLevel |
| 860 | CAD ~ Intercept.High + Age + APOB + corin + LowLevel |
| 861 | CAD ~ Intercept.High + Age + APOB + corin + LowLevel |
| 862 | CAD ~ Intercept.High + Age + APOB + corin + LowLevel |
| 863 | CAD ~ Intercept.High + Age + APOB + corin + LowLevel |
| 864 | CAD ~ Intercept.High + Age + APOB + corin + LowLevel |
| 865 | CAD ~ Intercept.High + Age + APOB + HSP70 + RBP4 + SERPINA12 + LowLevel |
| 866 | CAD ~ Intercept.High + Age + APOB + HSP70 + RBP4 + SERPINA12 + LowLevel |
| 867 | CAD ~ Intercept.High + Age + APOB + HSP70 + RBP4 + SERPINA12 + LowLevel |
| 868 | CAD ~ Intercept.High + Age + APOB + HSP70 + RBP4 + SERPINA12 + LowLevel |
| 869 | CAD ~ Intercept.High + Age + APOB + HSP70 + RBP4 + SERPINA12 + LowLevel |
| 870 | CAD ~ Intercept.High + Age + APOB + HSP70 + RBP4 + SERPINA12 + LowLevel |
| 871 | CAD ~ Intercept.High + Age + APOB + HSP70 + RBP4 + SERPINA12 + LowLevel |
| 872 | CAD ~ Intercept.High + Age + APOB + HSP70 + RBP4 + SERPINA12 + LowLevel |
| 873 | CAD ~ Intercept.High + Age + APOB + HSP70 + RBP4 + SERPINA12 + LowLevel |
| 874 | CAD ~ Intercept.High + Age + APOB + HSP70 + RBP4 + SERPINA12 + LowLevel |
| 875 | CAD ~ Intercept.High + Age + APOB + HSP70 + RBP4 + LowLevel |
| 876 | CAD ~ Intercept.High + Age + APOB + HSP70 + RBP4 + LowLevel |
| 877 | CAD ~ Intercept.High + Age + APOB + HSP70 + RBP4 + LowLevel |
| 878 | CAD ~ Intercept.High + Age + APOB + HSP70 + RBP4 + LowLevel |
| 879 | CAD ~ Intercept.High + Age + APOB + HSP70 + RBP4 + LowLevel |
| 880 | CAD ~ Intercept.High + Age + APOB + HSP70 + RBP4 + LowLevel |
| 881 | CAD ~ Intercept.High + Age + APOB + HSP70 + RBP4 + LowLevel |
| 882 | CAD ~ Intercept.High + Age + APOB + HSP70 + RBP4 + LowLevel |
| 883 | CAD ~ Intercept.High + Age + APOB + HSP70 + RBP4 + LowLevel |
| 884 | CAD ~ Intercept.High + Age + APOB + HSP70 + RBP4 + LowLevel |
| 885 | CAD ~ Intercept.High + Age + APOB + HSP70 + SERPINA12 + LowLevel |
| 886 | CAD ~ Intercept.High + Age + APOB + HSP70 + SERPINA12 + LowLevel |
| 887 | CAD ~ Intercept.High + Age + APOB + HSP70 + SERPINA12 + LowLevel |
| 888 | CAD ~ Intercept.High + Age + APOB + HSP70 + SERPINA12 + LowLevel |
| 889 | CAD ~ Intercept.High + Age + APOB + HSP70 + SERPINA12 + LowLevel |
| 890 | CAD ~ Intercept.High + Age + APOB + HSP70 + SERPINA12 + LowLevel |
| 891 | CAD ~ Intercept.High + Age + APOB + HSP70 + SERPINA12 + LowLevel |
| 892 | CAD ~ Intercept.High + Age + APOB + HSP70 + SERPINA12 + LowLevel |
| 893 | CAD ~ Intercept.High + Age + APOB + HSP70 + SERPINA12 + LowLevel |
| 894 | CAD ~ Intercept.High + Age + APOB + HSP70 + SERPINA12 + LowLevel |
| 895 | CAD ~ Intercept.High + Age + APOB + HSP70 + LowLevel |
| 896 | CAD ~ Intercept.High + Age + APOB + HSP70 + LowLevel |
| 897 | CAD ~ Intercept.High + Age + APOB + HSP70 + LowLevel |
| 898 | CAD ~ Intercept.High + Age + APOB + HSP70 + LowLevel |
| 899 | CAD ~ Intercept.High + Age + APOB + HSP70 + LowLevel |
| 900 | CAD ~ Intercept.High + Age + APOB + RBP4 + SERPINA12 + LowLevel |
| 901 | CAD ~ Intercept.High + Age + APOB + RBP4 + SERPINA12 + LowLevel |
| 902 | CAD ~ Intercept.High + Age + APOB + RBP4 + SERPINA12 + LowLevel |
| 903 | CAD ~ Intercept.High + Age + APOB + RBP4 + SERPINA12 + LowLevel |
| 904 | CAD ~ Intercept.High + Age + APOB + RBP4 + SERPINA12 + LowLevel |
| 905 | CAD ~ Intercept.High + Age + APOB + RBP4 + SERPINA12 + LowLevel |
| 906 | CAD ~ Intercept.High + Age + APOB + RBP4 + SERPINA12 + LowLevel |
| 907 | CAD ~ Intercept.High + Age + APOB + RBP4 + SERPINA12 + LowLevel |
| 908 | CAD ~ Intercept.High + Age + APOB + RBP4 + SERPINA12 + LowLevel |
| 909 | CAD ~ Intercept.High + Age + APOB + RBP4 + SERPINA12 + LowLevel |
| 910 | CAD ~ Intercept.High + Age + APOB + RBP4 + LowLevel |
| 911 | CAD ~ Intercept.High + Age + APOB + RBP4 + LowLevel |
| 912 | CAD ~ Intercept.High + Age + APOB + RBP4 + LowLevel |
| 913 | CAD ~ Intercept.High + Age + APOB + RBP4 + LowLevel |
| 914 | CAD ~ Intercept.High + Age + APOB + RBP4 + LowLevel |
| 915 | CAD ~ Intercept.High + Age + APOB + SERPINA12 + LowLevel |
| 916 | CAD ~ Intercept.High + Age + APOB + SERPINA12 + LowLevel |
| 917 | CAD ~ Intercept.High + Age + APOB + SERPINA12 + LowLevel |
| 918 | CAD ~ Intercept.High + Age + APOB + SERPINA12 + LowLevel |
| 919 | CAD ~ Intercept.High + Age + APOB + SERPINA12 + LowLevel |
| 920 | CAD ~ Intercept.High + Age + APOB + LowLevel |
| 921 | CAD ~ Intercept.High + Age + corin + HSP70 + RBP4 + SERPINA12 + LowLevel |
| 922 | CAD ~ Intercept.High + Age + corin + HSP70 + RBP4 + SERPINA12 + LowLevel |
| 923 | CAD ~ Intercept.High + Age + corin + HSP70 + RBP4 + SERPINA12 + LowLevel |
| 924 | CAD ~ Intercept.High + Age + corin + HSP70 + RBP4 + SERPINA12 + LowLevel |
| 925 | CAD ~ Intercept.High + Age + corin + HSP70 + RBP4 + SERPINA12 + LowLevel |
| 926 | CAD ~ Intercept.High + Age + corin + HSP70 + RBP4 + SERPINA12 + LowLevel |
| 927 | CAD ~ Intercept.High + Age + corin + HSP70 + RBP4 + SERPINA12 + LowLevel |
| 928 | CAD ~ Intercept.High + Age + corin + HSP70 + RBP4 + SERPINA12 + LowLevel |
| 929 | CAD ~ Intercept.High + Age + corin + HSP70 + RBP4 + SERPINA12 + LowLevel |
| 930 | CAD ~ Intercept.High + Age + corin + HSP70 + RBP4 + SERPINA12 + LowLevel |
| 931 | CAD ~ Intercept.High + Age + corin + HSP70 + RBP4 + LowLevel |
| 932 | CAD ~ Intercept.High + Age + corin + HSP70 + RBP4 + LowLevel |
| 933 | CAD ~ Intercept.High + Age + corin + HSP70 + RBP4 + LowLevel |
| 934 | CAD ~ Intercept.High + Age + corin + HSP70 + RBP4 + LowLevel |
| 935 | CAD ~ Intercept.High + Age + corin + HSP70 + RBP4 + LowLevel |
| 936 | CAD ~ Intercept.High + Age + corin + HSP70 + RBP4 + LowLevel |
| 937 | CAD ~ Intercept.High + Age + corin + HSP70 + RBP4 + LowLevel |
| 938 | CAD ~ Intercept.High + Age + corin + HSP70 + RBP4 + LowLevel |
| 939 | CAD ~ Intercept.High + Age + corin + HSP70 + RBP4 + LowLevel |
| 940 | CAD ~ Intercept.High + Age + corin + HSP70 + RBP4 + LowLevel |
| 941 | CAD ~ Intercept.High + Age + corin + HSP70 + SERPINA12 + LowLevel |
| 942 | CAD ~ Intercept.High + Age + corin + HSP70 + SERPINA12 + LowLevel |
| 943 | CAD ~ Intercept.High + Age + corin + HSP70 + SERPINA12 + LowLevel |
| 944 | CAD ~ Intercept.High + Age + corin + HSP70 + SERPINA12 + LowLevel |
| 945 | CAD ~ Intercept.High + Age + corin + HSP70 + SERPINA12 + LowLevel |
| 946 | CAD ~ Intercept.High + Age + corin + HSP70 + SERPINA12 + LowLevel |
| 947 | CAD ~ Intercept.High + Age + corin + HSP70 + SERPINA12 + LowLevel |
| 948 | CAD ~ Intercept.High + Age + corin + HSP70 + SERPINA12 + LowLevel |
| 949 | CAD ~ Intercept.High + Age + corin + HSP70 + SERPINA12 + LowLevel |
| 950 | CAD ~ Intercept.High + Age + corin + HSP70 + SERPINA12 + LowLevel |
| 951 | CAD ~ Intercept.High + Age + corin + HSP70 + LowLevel |
| 952 | CAD ~ Intercept.High + Age + corin + HSP70 + LowLevel |
| 953 | CAD ~ Intercept.High + Age + corin + HSP70 + LowLevel |
| 954 | CAD ~ Intercept.High + Age + corin + HSP70 + LowLevel |
| 955 | CAD ~ Intercept.High + Age + corin + HSP70 + LowLevel |
| 956 | CAD ~ Intercept.High + Age + corin + RBP4 + SERPINA12 + LowLevel |
| 957 | CAD ~ Intercept.High + Age + corin + RBP4 + SERPINA12 + LowLevel |
| 958 | CAD ~ Intercept.High + Age + corin + RBP4 + SERPINA12 + LowLevel |
| 959 | CAD ~ Intercept.High + Age + corin + RBP4 + SERPINA12 + LowLevel |
| 960 | CAD ~ Intercept.High + Age + corin + RBP4 + SERPINA12 + LowLevel |
| 961 | CAD ~ Intercept.High + Age + corin + RBP4 + SERPINA12 + LowLevel |
| 962 | CAD ~ Intercept.High + Age + corin + RBP4 + SERPINA12 + LowLevel |
| 963 | CAD ~ Intercept.High + Age + corin + RBP4 + SERPINA12 + LowLevel |
| 964 | CAD ~ Intercept.High + Age + corin + RBP4 + SERPINA12 + LowLevel |
| 965 | CAD ~ Intercept.High + Age + corin + RBP4 + SERPINA12 + LowLevel |
| 966 | CAD ~ Intercept.High + Age + corin + RBP4 + LowLevel |
| 967 | CAD ~ Intercept.High + Age + corin + RBP4 + LowLevel |
| 968 | CAD ~ Intercept.High + Age + corin + RBP4 + LowLevel |
| 969 | CAD ~ Intercept.High + Age + corin + RBP4 + LowLevel |
| 970 | CAD ~ Intercept.High + Age + corin + RBP4 + LowLevel |
| 971 | CAD ~ Intercept.High + Age + corin + SERPINA12 + LowLevel |
| 972 | CAD ~ Intercept.High + Age + corin + SERPINA12 + LowLevel |
| 973 | CAD ~ Intercept.High + Age + corin + SERPINA12 + LowLevel |
| 974 | CAD ~ Intercept.High + Age + corin + SERPINA12 + LowLevel |
| 975 | CAD ~ Intercept.High + Age + corin + SERPINA12 + LowLevel |
| 976 | CAD ~ Intercept.High + Age + corin + LowLevel |
| 977 | CAD ~ Intercept.High + Age + HSP70 + RBP4 + SERPINA12 + LowLevel |
| 978 | CAD ~ Intercept.High + Age + HSP70 + RBP4 + SERPINA12 + LowLevel |
| 979 | CAD ~ Intercept.High + Age + HSP70 + RBP4 + SERPINA12 + LowLevel |
| 980 | CAD ~ Intercept.High + Age + HSP70 + RBP4 + SERPINA12 + LowLevel |
| 981 | CAD ~ Intercept.High + Age + HSP70 + RBP4 + SERPINA12 + LowLevel |
| 982 | CAD ~ Intercept.High + Age + HSP70 + RBP4 + SERPINA12 + LowLevel |
| 983 | CAD ~ Intercept.High + Age + HSP70 + RBP4 + SERPINA12 + LowLevel |
| 984 | CAD ~ Intercept.High + Age + HSP70 + RBP4 + SERPINA12 + LowLevel |
| 985 | CAD ~ Intercept.High + Age + HSP70 + RBP4 + SERPINA12 + LowLevel |
| 986 | CAD ~ Intercept.High + Age + HSP70 + RBP4 + SERPINA12 + LowLevel |
| 987 | CAD ~ Intercept.High + Age + HSP70 + RBP4 + LowLevel |
| 988 | CAD ~ Intercept.High + Age + HSP70 + RBP4 + LowLevel |
| 989 | CAD ~ Intercept.High + Age + HSP70 + RBP4 + LowLevel |
| 990 | CAD ~ Intercept.High + Age + HSP70 + RBP4 + LowLevel |
| 991 | CAD ~ Intercept.High + Age + HSP70 + RBP4 + LowLevel |
| 992 | CAD ~ Intercept.High + Age + HSP70 + SERPINA12 + LowLevel |
| 993 | CAD ~ Intercept.High + Age + HSP70 + SERPINA12 + LowLevel |
| 994 | CAD ~ Intercept.High + Age + HSP70 + SERPINA12 + LowLevel |
| 995 | CAD ~ Intercept.High + Age + HSP70 + SERPINA12 + LowLevel |
| 996 | CAD ~ Intercept.High + Age + HSP70 + SERPINA12 + LowLevel |
| 997 | CAD ~ Intercept.High + Age + HSP70 + LowLevel |
| 998 | CAD ~ Intercept.High + Age + RBP4 + SERPINA12 + LowLevel |
| 999 | CAD ~ Intercept.High + Age + RBP4 + SERPINA12 + LowLevel |
| 1000 | CAD ~ Intercept.High + Age + RBP4 + SERPINA12 + LowLevel |
| 1001 | CAD ~ Intercept.High + Age + RBP4 + SERPINA12 + LowLevel |
| 1002 | CAD ~ Intercept.High + Age + RBP4 + SERPINA12 + LowLevel |
| 1003 | CAD ~ Intercept.High + Age + RBP4 + LowLevel |
| 1004 | CAD ~ Intercept.High + Age + SERPINA12 + LowLevel |
| 1005 | CAD ~ Intercept.High + APOB + corin + HSP70 + RBP4 + SERPINA12 + LowLevel |
| 1006 | CAD ~ Intercept.High + APOB + corin + HSP70 + RBP4 + SERPINA12 + LowLevel |
| 1007 | CAD ~ Intercept.High + APOB + corin + HSP70 + RBP4 + SERPINA12 + LowLevel |
| 1008 | CAD ~ Intercept.High + APOB + corin + HSP70 + RBP4 + SERPINA12 + LowLevel |
| 1009 | CAD ~ Intercept.High + APOB + corin + HSP70 + RBP4 + SERPINA12 + LowLevel |
| 1010 | CAD ~ Intercept.High + APOB + corin + HSP70 + RBP4 + SERPINA12 + LowLevel |
| 1011 | CAD ~ Intercept.High + APOB + corin + HSP70 + RBP4 + SERPINA12 + LowLevel |
| 1012 | CAD ~ Intercept.High + APOB + corin + HSP70 + RBP4 + SERPINA12 + LowLevel |
| 1013 | CAD ~ Intercept.High + APOB + corin + HSP70 + RBP4 + SERPINA12 + LowLevel |
| 1014 | CAD ~ Intercept.High + APOB + corin + HSP70 + RBP4 + SERPINA12 + LowLevel |
| 1015 | CAD ~ Intercept.High + APOB + corin + HSP70 + RBP4 + LowLevel |
| 1016 | CAD ~ Intercept.High + APOB + corin + HSP70 + RBP4 + LowLevel |
| 1017 | CAD ~ Intercept.High + APOB + corin + HSP70 + RBP4 + LowLevel |
| 1018 | CAD ~ Intercept.High + APOB + corin + HSP70 + RBP4 + LowLevel |
| 1019 | CAD ~ Intercept.High + APOB + corin + HSP70 + RBP4 + LowLevel |
| 1020 | CAD ~ Intercept.High + APOB + corin + HSP70 + RBP4 + LowLevel |
| 1021 | CAD ~ Intercept.High + APOB + corin + HSP70 + RBP4 + LowLevel |
| 1022 | CAD ~ Intercept.High + APOB + corin + HSP70 + RBP4 + LowLevel |
| 1023 | CAD ~ Intercept.High + APOB + corin + HSP70 + RBP4 + LowLevel |
| 1024 | CAD ~ Intercept.High + APOB + corin + HSP70 + RBP4 + LowLevel |
| 1025 | CAD ~ Intercept.High + APOB + corin + HSP70 + SERPINA12 + LowLevel |
| 1026 | CAD ~ Intercept.High + APOB + corin + HSP70 + SERPINA12 + LowLevel |
| 1027 | CAD ~ Intercept.High + APOB + corin + HSP70 + SERPINA12 + LowLevel |
| 1028 | CAD ~ Intercept.High + APOB + corin + HSP70 + SERPINA12 + LowLevel |
| 1029 | CAD ~ Intercept.High + APOB + corin + HSP70 + SERPINA12 + LowLevel |
| 1030 | CAD ~ Intercept.High + APOB + corin + HSP70 + SERPINA12 + LowLevel |
| 1031 | CAD ~ Intercept.High + APOB + corin + HSP70 + SERPINA12 + LowLevel |
| 1032 | CAD ~ Intercept.High + APOB + corin + HSP70 + SERPINA12 + LowLevel |
| 1033 | CAD ~ Intercept.High + APOB + corin + HSP70 + SERPINA12 + LowLevel |
| 1034 | CAD ~ Intercept.High + APOB + corin + HSP70 + SERPINA12 + LowLevel |
| 1035 | CAD ~ Intercept.High + APOB + corin + HSP70 + LowLevel |
| 1036 | CAD ~ Intercept.High + APOB + corin + HSP70 + LowLevel |
| 1037 | CAD ~ Intercept.High + APOB + corin + HSP70 + LowLevel |
| 1038 | CAD ~ Intercept.High + APOB + corin + HSP70 + LowLevel |
| 1039 | CAD ~ Intercept.High + APOB + corin + HSP70 + LowLevel |
| 1040 | CAD ~ Intercept.High + APOB + corin + RBP4 + SERPINA12 + LowLevel |
| 1041 | CAD ~ Intercept.High + APOB + corin + RBP4 + SERPINA12 + LowLevel |
| 1042 | CAD ~ Intercept.High + APOB + corin + RBP4 + SERPINA12 + LowLevel |
| 1043 | CAD ~ Intercept.High + APOB + corin + RBP4 + SERPINA12 + LowLevel |
| 1044 | CAD ~ Intercept.High + APOB + corin + RBP4 + SERPINA12 + LowLevel |
| 1045 | CAD ~ Intercept.High + APOB + corin + RBP4 + SERPINA12 + LowLevel |
| 1046 | CAD ~ Intercept.High + APOB + corin + RBP4 + SERPINA12 + LowLevel |
| 1047 | CAD ~ Intercept.High + APOB + corin + RBP4 + SERPINA12 + LowLevel |
| 1048 | CAD ~ Intercept.High + APOB + corin + RBP4 + SERPINA12 + LowLevel |
| 1049 | CAD ~ Intercept.High + APOB + corin + RBP4 + SERPINA12 + LowLevel |
| 1050 | CAD ~ Intercept.High + APOB + corin + RBP4 + LowLevel |
| 1051 | CAD ~ Intercept.High + APOB + corin + RBP4 + LowLevel |
| 1052 | CAD ~ Intercept.High + APOB + corin + RBP4 + LowLevel |
| 1053 | CAD ~ Intercept.High + APOB + corin + RBP4 + LowLevel |
| 1054 | CAD ~ Intercept.High + APOB + corin + RBP4 + LowLevel |
| 1055 | CAD ~ Intercept.High + APOB + corin + SERPINA12 + LowLevel |
| 1056 | CAD ~ Intercept.High + APOB + corin + SERPINA12 + LowLevel |
| 1057 | CAD ~ Intercept.High + APOB + corin + SERPINA12 + LowLevel |
| 1058 | CAD ~ Intercept.High + APOB + corin + SERPINA12 + LowLevel |
| 1059 | CAD ~ Intercept.High + APOB + corin + SERPINA12 + LowLevel |
| 1060 | CAD ~ Intercept.High + APOB + corin + LowLevel |
| 1061 | CAD ~ Intercept.High + APOB + HSP70 + RBP4 + SERPINA12 + LowLevel |
| 1062 | CAD ~ Intercept.High + APOB + HSP70 + RBP4 + SERPINA12 + LowLevel |
| 1063 | CAD ~ Intercept.High + APOB + HSP70 + RBP4 + SERPINA12 + LowLevel |
| 1064 | CAD ~ Intercept.High + APOB + HSP70 + RBP4 + SERPINA12 + LowLevel |
| 1065 | CAD ~ Intercept.High + APOB + HSP70 + RBP4 + SERPINA12 + LowLevel |
| 1066 | CAD ~ Intercept.High + APOB + HSP70 + RBP4 + SERPINA12 + LowLevel |
| 1067 | CAD ~ Intercept.High + APOB + HSP70 + RBP4 + SERPINA12 + LowLevel |
| 1068 | CAD ~ Intercept.High + APOB + HSP70 + RBP4 + SERPINA12 + LowLevel |
| 1069 | CAD ~ Intercept.High + APOB + HSP70 + RBP4 + SERPINA12 + LowLevel |
| 1070 | CAD ~ Intercept.High + APOB + HSP70 + RBP4 + SERPINA12 + LowLevel |
| 1071 | CAD ~ Intercept.High + APOB + HSP70 + RBP4 + LowLevel |
| 1072 | CAD ~ Intercept.High + APOB + HSP70 + RBP4 + LowLevel |
| 1073 | CAD ~ Intercept.High + APOB + HSP70 + RBP4 + LowLevel |
| 1074 | CAD ~ Intercept.High + APOB + HSP70 + RBP4 + LowLevel |
| 1075 | CAD ~ Intercept.High + APOB + HSP70 + RBP4 + LowLevel |
| 1076 | CAD ~ Intercept.High + APOB + HSP70 + SERPINA12 + LowLevel |
| 1077 | CAD ~ Intercept.High + APOB + HSP70 + SERPINA12 + LowLevel |
| 1078 | CAD ~ Intercept.High + APOB + HSP70 + SERPINA12 + LowLevel |
| 1079 | CAD ~ Intercept.High + APOB + HSP70 + SERPINA12 + LowLevel |
| 1080 | CAD ~ Intercept.High + APOB + HSP70 + SERPINA12 + LowLevel |
| 1081 | CAD ~ Intercept.High + APOB + HSP70 + LowLevel |
| 1082 | CAD ~ Intercept.High + APOB + RBP4 + SERPINA12 + LowLevel |
| 1083 | CAD ~ Intercept.High + APOB + RBP4 + SERPINA12 + LowLevel |
| 1084 | CAD ~ Intercept.High + APOB + RBP4 + SERPINA12 + LowLevel |
| 1085 | CAD ~ Intercept.High + APOB + RBP4 + SERPINA12 + LowLevel |
| 1086 | CAD ~ Intercept.High + APOB + RBP4 + SERPINA12 + LowLevel |
| 1087 | CAD ~ Intercept.High + APOB + RBP4 + LowLevel |
| 1088 | CAD ~ Intercept.High + APOB + SERPINA12 + LowLevel |
| 1089 | CAD ~ Intercept.High + corin + HSP70 + RBP4 + SERPINA12 + LowLevel |
| 1090 | CAD ~ Intercept.High + corin + HSP70 + RBP4 + SERPINA12 + LowLevel |
| 1091 | CAD ~ Intercept.High + corin + HSP70 + RBP4 + SERPINA12 + LowLevel |
| 1092 | CAD ~ Intercept.High + corin + HSP70 + RBP4 + SERPINA12 + LowLevel |
| 1093 | CAD ~ Intercept.High + corin + HSP70 + RBP4 + SERPINA12 + LowLevel |
| 1094 | CAD ~ Intercept.High + corin + HSP70 + RBP4 + SERPINA12 + LowLevel |
| 1095 | CAD ~ Intercept.High + corin + HSP70 + RBP4 + SERPINA12 + LowLevel |
| 1096 | CAD ~ Intercept.High + corin + HSP70 + RBP4 + SERPINA12 + LowLevel |
| 1097 | CAD ~ Intercept.High + corin + HSP70 + RBP4 + SERPINA12 + LowLevel |
| 1098 | CAD ~ Intercept.High + corin + HSP70 + RBP4 + SERPINA12 + LowLevel |
| 1099 | CAD ~ Intercept.High + corin + HSP70 + RBP4 + LowLevel |
| 1100 | CAD ~ Intercept.High + corin + HSP70 + RBP4 + LowLevel |
| 1101 | CAD ~ Intercept.High + corin + HSP70 + RBP4 + LowLevel |
| 1102 | CAD ~ Intercept.High + corin + HSP70 + RBP4 + LowLevel |
| 1103 | CAD ~ Intercept.High + corin + HSP70 + RBP4 + LowLevel |
| 1104 | CAD ~ Intercept.High + corin + HSP70 + SERPINA12 + LowLevel |
| 1105 | CAD ~ Intercept.High + corin + HSP70 + SERPINA12 + LowLevel |
| 1106 | CAD ~ Intercept.High + corin + HSP70 + SERPINA12 + LowLevel |
| 1107 | CAD ~ Intercept.High + corin + HSP70 + SERPINA12 + LowLevel |
| 1108 | CAD ~ Intercept.High + corin + HSP70 + SERPINA12 + LowLevel |
| 1109 | CAD ~ Intercept.High + corin + HSP70 + LowLevel |
| 1110 | CAD ~ Intercept.High + corin + RBP4 + SERPINA12 + LowLevel |
| 1111 | CAD ~ Intercept.High + corin + RBP4 + SERPINA12 + LowLevel |
| 1112 | CAD ~ Intercept.High + corin + RBP4 + SERPINA12 + LowLevel |
| 1113 | CAD ~ Intercept.High + corin + RBP4 + SERPINA12 + LowLevel |
| 1114 | CAD ~ Intercept.High + corin + RBP4 + SERPINA12 + LowLevel |
| 1115 | CAD ~ Intercept.High + corin + RBP4 + LowLevel |
| 1116 | CAD ~ Intercept.High + corin + SERPINA12 + LowLevel |
| 1117 | CAD ~ Intercept.High + Sex + Age + APOB + corin + HSP70 + RBP4 + LowLevel |
| 1118 | CAD ~ Intercept.High + Sex + Age + APOB + corin + HSP70 + RBP4 + LowLevel |
| 1119 | CAD ~ Intercept.High + Sex + Age + APOB + corin + HSP70 + RBP4 + LowLevel |
| 1120 | CAD ~ Intercept.High + Sex + Age + APOB + corin + HSP70 + RBP4 + LowLevel |
| 1121 | CAD ~ Intercept.High + Sex + Age + APOB + corin + HSP70 + RBP4 + LowLevel |
| 1122 | CAD ~ Intercept.High + Sex + Age + APOB + corin + HSP70 + SERPINA12 + LowLevel |
| 1123 | CAD ~ Intercept.High + Sex + Age + APOB + corin + HSP70 + SERPINA12 + LowLevel |
| 1124 | CAD ~ Intercept.High + Sex + Age + APOB + corin + HSP70 + SERPINA12 + LowLevel |
| 1125 | CAD ~ Intercept.High + Sex + Age + APOB + corin + HSP70 + SERPINA12 + LowLevel |
| 1126 | CAD ~ Intercept.High + Sex + Age + APOB + corin + HSP70 + SERPINA12 + LowLevel |
| 1127 | CAD ~ Intercept.High + Sex + Age + APOB + corin + HSP70 + LowLevel |
| 1128 | CAD ~ Intercept.High + Sex + Age + APOB + corin + HSP70 + LowLevel |
| 1129 | CAD ~ Intercept.High + Sex + Age + APOB + corin + HSP70 + LowLevel |
| 1130 | CAD ~ Intercept.High + Sex + Age + APOB + corin + HSP70 + LowLevel |
| 1131 | CAD ~ Intercept.High + Sex + Age + APOB + corin + HSP70 + LowLevel |
| 1132 | CAD ~ Intercept.High + Sex + Age + APOB + corin + HSP70 + LowLevel |
| 1133 | CAD ~ Intercept.High + Sex + Age + APOB + corin + HSP70 + LowLevel |
| 1134 | CAD ~ Intercept.High + Sex + Age + APOB + corin + HSP70 + LowLevel |
| 1135 | CAD ~ Intercept.High + Sex + Age + APOB + corin + HSP70 + LowLevel |
| 1136 | CAD ~ Intercept.High + Sex + Age + APOB + corin + HSP70 + LowLevel |
| 1137 | CAD ~ Intercept.High + Sex + Age + APOB + corin + RBP4 + SERPINA12 + LowLevel |
| 1138 | CAD ~ Intercept.High + Sex + Age + APOB + corin + RBP4 + SERPINA12 + LowLevel |
| 1139 | CAD ~ Intercept.High + Sex + Age + APOB + corin + RBP4 + SERPINA12 + LowLevel |
| 1140 | CAD ~ Intercept.High + Sex + Age + APOB + corin + RBP4 + SERPINA12 + LowLevel |
| 1141 | CAD ~ Intercept.High + Sex + Age + APOB + corin + RBP4 + SERPINA12 + LowLevel |
| 1142 | CAD ~ Intercept.High + Sex + Age + APOB + corin + RBP4 + LowLevel |
| 1143 | CAD ~ Intercept.High + Sex + Age + APOB + corin + RBP4 + LowLevel |
| 1144 | CAD ~ Intercept.High + Sex + Age + APOB + corin + RBP4 + LowLevel |
| 1145 | CAD ~ Intercept.High + Sex + Age + APOB + corin + RBP4 + LowLevel |
| 1146 | CAD ~ Intercept.High + Sex + Age + APOB + corin + RBP4 + LowLevel |
| 1147 | CAD ~ Intercept.High + Sex + Age + APOB + corin + RBP4 + LowLevel |
| 1148 | CAD ~ Intercept.High + Sex + Age + APOB + corin + RBP4 + LowLevel |
| 1149 | CAD ~ Intercept.High + Sex + Age + APOB + corin + RBP4 + LowLevel |
| 1150 | CAD ~ Intercept.High + Sex + Age + APOB + corin + RBP4 + LowLevel |
| 1151 | CAD ~ Intercept.High + Sex + Age + APOB + corin + RBP4 + LowLevel |
| 1152 | CAD ~ Intercept.High + Sex + Age + APOB + corin + SERPINA12 + LowLevel |
| 1153 | CAD ~ Intercept.High + Sex + Age + APOB + corin + SERPINA12 + LowLevel |
| 1154 | CAD ~ Intercept.High + Sex + Age + APOB + corin + SERPINA12 + LowLevel |
| 1155 | CAD ~ Intercept.High + Sex + Age + APOB + corin + SERPINA12 + LowLevel |
| 1156 | CAD ~ Intercept.High + Sex + Age + APOB + corin + SERPINA12 + LowLevel |
| 1157 | CAD ~ Intercept.High + Sex + Age + APOB + corin + SERPINA12 + LowLevel |
| 1158 | CAD ~ Intercept.High + Sex + Age + APOB + corin + SERPINA12 + LowLevel |
| 1159 | CAD ~ Intercept.High + Sex + Age + APOB + corin + SERPINA12 + LowLevel |
| 1160 | CAD ~ Intercept.High + Sex + Age + APOB + corin + SERPINA12 + LowLevel |
| 1161 | CAD ~ Intercept.High + Sex + Age + APOB + corin + SERPINA12 + LowLevel |
| 1162 | CAD ~ Intercept.High + Sex + Age + APOB + corin + LowLevel |
| 1163 | CAD ~ Intercept.High + Sex + Age + APOB + corin + LowLevel |
| 1164 | CAD ~ Intercept.High + Sex + Age + APOB + corin + LowLevel |
| 1165 | CAD ~ Intercept.High + Sex + Age + APOB + corin + LowLevel |
| 1166 | CAD ~ Intercept.High + Sex + Age + APOB + corin + LowLevel |
| 1167 | CAD ~ Intercept.High + Sex + Age + APOB + corin + LowLevel |
| 1168 | CAD ~ Intercept.High + Sex + Age + APOB + corin + LowLevel |
| 1169 | CAD ~ Intercept.High + Sex + Age + APOB + corin + LowLevel |
| 1170 | CAD ~ Intercept.High + Sex + Age + APOB + corin + LowLevel |
| 1171 | CAD ~ Intercept.High + Sex + Age + APOB + corin + LowLevel |
| 1172 | CAD ~ Intercept.High + Sex + Age + APOB + HSP70 + RBP4 + SERPINA12 + LowLevel |
| 1173 | CAD ~ Intercept.High + Sex + Age + APOB + HSP70 + RBP4 + SERPINA12 + LowLevel |
| 1174 | CAD ~ Intercept.High + Sex + Age + APOB + HSP70 + RBP4 + SERPINA12 + LowLevel |
| 1175 | CAD ~ Intercept.High + Sex + Age + APOB + HSP70 + RBP4 + SERPINA12 + LowLevel |
| 1176 | CAD ~ Intercept.High + Sex + Age + APOB + HSP70 + RBP4 + SERPINA12 + LowLevel |
| 1177 | CAD ~ Intercept.High + Sex + Age + APOB + HSP70 + RBP4 + LowLevel |
| 1178 | CAD ~ Intercept.High + Sex + Age + APOB + HSP70 + RBP4 + LowLevel |
| 1179 | CAD ~ Intercept.High + Sex + Age + APOB + HSP70 + RBP4 + LowLevel |
| 1180 | CAD ~ Intercept.High + Sex + Age + APOB + HSP70 + RBP4 + LowLevel |
| 1181 | CAD ~ Intercept.High + Sex + Age + APOB + HSP70 + RBP4 + LowLevel |
| 1182 | CAD ~ Intercept.High + Sex + Age + APOB + HSP70 + RBP4 + LowLevel |
| 1183 | CAD ~ Intercept.High + Sex + Age + APOB + HSP70 + RBP4 + LowLevel |
| 1184 | CAD ~ Intercept.High + Sex + Age + APOB + HSP70 + RBP4 + LowLevel |
| 1185 | CAD ~ Intercept.High + Sex + Age + APOB + HSP70 + RBP4 + LowLevel |
| 1186 | CAD ~ Intercept.High + Sex + Age + APOB + HSP70 + RBP4 + LowLevel |
| 1187 | CAD ~ Intercept.High + Sex + Age + APOB + HSP70 + SERPINA12 + LowLevel |
| 1188 | CAD ~ Intercept.High + Sex + Age + APOB + HSP70 + SERPINA12 + LowLevel |
| 1189 | CAD ~ Intercept.High + Sex + Age + APOB + HSP70 + SERPINA12 + LowLevel |
| 1190 | CAD ~ Intercept.High + Sex + Age + APOB + HSP70 + SERPINA12 + LowLevel |
| 1191 | CAD ~ Intercept.High + Sex + Age + APOB + HSP70 + SERPINA12 + LowLevel |
| 1192 | CAD ~ Intercept.High + Sex + Age + APOB + HSP70 + SERPINA12 + LowLevel |
| 1193 | CAD ~ Intercept.High + Sex + Age + APOB + HSP70 + SERPINA12 + LowLevel |
| 1194 | CAD ~ Intercept.High + Sex + Age + APOB + HSP70 + SERPINA12 + LowLevel |
| 1195 | CAD ~ Intercept.High + Sex + Age + APOB + HSP70 + SERPINA12 + LowLevel |
| 1196 | CAD ~ Intercept.High + Sex + Age + APOB + HSP70 + SERPINA12 + LowLevel |
| 1197 | CAD ~ Intercept.High + Sex + Age + APOB + HSP70 + LowLevel |
| 1198 | CAD ~ Intercept.High + Sex + Age + APOB + HSP70 + LowLevel |
| 1199 | CAD ~ Intercept.High + Sex + Age + APOB + HSP70 + LowLevel |
| 1200 | CAD ~ Intercept.High + Sex + Age + APOB + HSP70 + LowLevel |
| 1201 | CAD ~ Intercept.High + Sex + Age + APOB + HSP70 + LowLevel |
| 1202 | CAD ~ Intercept.High + Sex + Age + APOB + HSP70 + LowLevel |
| 1203 | CAD ~ Intercept.High + Sex + Age + APOB + HSP70 + LowLevel |
| 1204 | CAD ~ Intercept.High + Sex + Age + APOB + HSP70 + LowLevel |
| 1205 | CAD ~ Intercept.High + Sex + Age + APOB + HSP70 + LowLevel |
| 1206 | CAD ~ Intercept.High + Sex + Age + APOB + HSP70 + LowLevel |
| 1207 | CAD ~ Intercept.High + Sex + Age + APOB + RBP4 + SERPINA12 + LowLevel |
| 1208 | CAD ~ Intercept.High + Sex + Age + APOB + RBP4 + SERPINA12 + LowLevel |
| 1209 | CAD ~ Intercept.High + Sex + Age + APOB + RBP4 + SERPINA12 + LowLevel |
| 1210 | CAD ~ Intercept.High + Sex + Age + APOB + RBP4 + SERPINA12 + LowLevel |
| 1211 | CAD ~ Intercept.High + Sex + Age + APOB + RBP4 + SERPINA12 + LowLevel |
| 1212 | CAD ~ Intercept.High + Sex + Age + APOB + RBP4 + SERPINA12 + LowLevel |
| 1213 | CAD ~ Intercept.High + Sex + Age + APOB + RBP4 + SERPINA12 + LowLevel |
| 1214 | CAD ~ Intercept.High + Sex + Age + APOB + RBP4 + SERPINA12 + LowLevel |
| 1215 | CAD ~ Intercept.High + Sex + Age + APOB + RBP4 + SERPINA12 + LowLevel |
| 1216 | CAD ~ Intercept.High + Sex + Age + APOB + RBP4 + SERPINA12 + LowLevel |
| 1217 | CAD ~ Intercept.High + Sex + Age + APOB + RBP4 + LowLevel |
| 1218 | CAD ~ Intercept.High + Sex + Age + APOB + RBP4 + LowLevel |
| 1219 | CAD ~ Intercept.High + Sex + Age + APOB + RBP4 + LowLevel |
| 1220 | CAD ~ Intercept.High + Sex + Age + APOB + RBP4 + LowLevel |
| 1221 | CAD ~ Intercept.High + Sex + Age + APOB + RBP4 + LowLevel |
| 1222 | CAD ~ Intercept.High + Sex + Age + APOB + RBP4 + LowLevel |
| 1223 | CAD ~ Intercept.High + Sex + Age + APOB + RBP4 + LowLevel |
| 1224 | CAD ~ Intercept.High + Sex + Age + APOB + RBP4 + LowLevel |
| 1225 | CAD ~ Intercept.High + Sex + Age + APOB + RBP4 + LowLevel |
| 1226 | CAD ~ Intercept.High + Sex + Age + APOB + RBP4 + LowLevel |
| 1227 | CAD ~ Intercept.High + Sex + Age + APOB + SERPINA12 + LowLevel |
| 1228 | CAD ~ Intercept.High + Sex + Age + APOB + SERPINA12 + LowLevel |
| 1229 | CAD ~ Intercept.High + Sex + Age + APOB + SERPINA12 + LowLevel |
| 1230 | CAD ~ Intercept.High + Sex + Age + APOB + SERPINA12 + LowLevel |
| 1231 | CAD ~ Intercept.High + Sex + Age + APOB + SERPINA12 + LowLevel |
| 1232 | CAD ~ Intercept.High + Sex + Age + APOB + SERPINA12 + LowLevel |
| 1233 | CAD ~ Intercept.High + Sex + Age + APOB + SERPINA12 + LowLevel |
| 1234 | CAD ~ Intercept.High + Sex + Age + APOB + SERPINA12 + LowLevel |
| 1235 | CAD ~ Intercept.High + Sex + Age + APOB + SERPINA12 + LowLevel |
| 1236 | CAD ~ Intercept.High + Sex + Age + APOB + SERPINA12 + LowLevel |
| 1237 | CAD ~ Intercept.High + Sex + Age + APOB + LowLevel |
| 1238 | CAD ~ Intercept.High + Sex + Age + APOB + LowLevel |
| 1239 | CAD ~ Intercept.High + Sex + Age + APOB + LowLevel |
| 1240 | CAD ~ Intercept.High + Sex + Age + APOB + LowLevel |
| 1241 | CAD ~ Intercept.High + Sex + Age + APOB + LowLevel |
| 1242 | CAD ~ Intercept.High + Sex + Age + corin + HSP70 + RBP4 + SERPINA12 + LowLevel |
| 1243 | CAD ~ Intercept.High + Sex + Age + corin + HSP70 + RBP4 + SERPINA12 + LowLevel |
| 1244 | CAD ~ Intercept.High + Sex + Age + corin + HSP70 + RBP4 + SERPINA12 + LowLevel |
| 1245 | CAD ~ Intercept.High + Sex + Age + corin + HSP70 + RBP4 + SERPINA12 + LowLevel |
| 1246 | CAD ~ Intercept.High + Sex + Age + corin + HSP70 + RBP4 + SERPINA12 + LowLevel |
| 1247 | CAD ~ Intercept.High + Sex + Age + corin + HSP70 + RBP4 + LowLevel |
| 1248 | CAD ~ Intercept.High + Sex + Age + corin + HSP70 + RBP4 + LowLevel |
| 1249 | CAD ~ Intercept.High + Sex + Age + corin + HSP70 + RBP4 + LowLevel |
| 1250 | CAD ~ Intercept.High + Sex + Age + corin + HSP70 + RBP4 + LowLevel |
| 1251 | CAD ~ Intercept.High + Sex + Age + corin + HSP70 + RBP4 + LowLevel |
| 1252 | CAD ~ Intercept.High + Sex + Age + corin + HSP70 + RBP4 + LowLevel |
| 1253 | CAD ~ Intercept.High + Sex + Age + corin + HSP70 + RBP4 + LowLevel |
| 1254 | CAD ~ Intercept.High + Sex + Age + corin + HSP70 + RBP4 + LowLevel |
| 1255 | CAD ~ Intercept.High + Sex + Age + corin + HSP70 + RBP4 + LowLevel |
| 1256 | CAD ~ Intercept.High + Sex + Age + corin + HSP70 + RBP4 + LowLevel |
| 1257 | CAD ~ Intercept.High + Sex + Age + corin + HSP70 + SERPINA12 + LowLevel |
| 1258 | CAD ~ Intercept.High + Sex + Age + corin + HSP70 + SERPINA12 + LowLevel |
| 1259 | CAD ~ Intercept.High + Sex + Age + corin + HSP70 + SERPINA12 + LowLevel |
| 1260 | CAD ~ Intercept.High + Sex + Age + corin + HSP70 + SERPINA12 + LowLevel |
| 1261 | CAD ~ Intercept.High + Sex + Age + corin + HSP70 + SERPINA12 + LowLevel |
| 1262 | CAD ~ Intercept.High + Sex + Age + corin + HSP70 + SERPINA12 + LowLevel |
| 1263 | CAD ~ Intercept.High + Sex + Age + corin + HSP70 + SERPINA12 + LowLevel |
| 1264 | CAD ~ Intercept.High + Sex + Age + corin + HSP70 + SERPINA12 + LowLevel |
| 1265 | CAD ~ Intercept.High + Sex + Age + corin + HSP70 + SERPINA12 + LowLevel |
| 1266 | CAD ~ Intercept.High + Sex + Age + corin + HSP70 + SERPINA12 + LowLevel |
| 1267 | CAD ~ Intercept.High + Sex + Age + corin + HSP70 + LowLevel |
| 1268 | CAD ~ Intercept.High + Sex + Age + corin + HSP70 + LowLevel |
| 1269 | CAD ~ Intercept.High + Sex + Age + corin + HSP70 + LowLevel |
| 1270 | CAD ~ Intercept.High + Sex + Age + corin + HSP70 + LowLevel |
| 1271 | CAD ~ Intercept.High + Sex + Age + corin + HSP70 + LowLevel |
| 1272 | CAD ~ Intercept.High + Sex + Age + corin + HSP70 + LowLevel |
| 1273 | CAD ~ Intercept.High + Sex + Age + corin + HSP70 + LowLevel |
| 1274 | CAD ~ Intercept.High + Sex + Age + corin + HSP70 + LowLevel |
| 1275 | CAD ~ Intercept.High + Sex + Age + corin + HSP70 + LowLevel |
| 1276 | CAD ~ Intercept.High + Sex + Age + corin + HSP70 + LowLevel |
| 1277 | CAD ~ Intercept.High + Sex + Age + corin + RBP4 + SERPINA12 + LowLevel |
| 1278 | CAD ~ Intercept.High + Sex + Age + corin + RBP4 + SERPINA12 + LowLevel |
| 1279 | CAD ~ Intercept.High + Sex + Age + corin + RBP4 + SERPINA12 + LowLevel |
| 1280 | CAD ~ Intercept.High + Sex + Age + corin + RBP4 + SERPINA12 + LowLevel |
| 1281 | CAD ~ Intercept.High + Sex + Age + corin + RBP4 + SERPINA12 + LowLevel |
| 1282 | CAD ~ Intercept.High + Sex + Age + corin + RBP4 + SERPINA12 + LowLevel |
| 1283 | CAD ~ Intercept.High + Sex + Age + corin + RBP4 + SERPINA12 + LowLevel |
| 1284 | CAD ~ Intercept.High + Sex + Age + corin + RBP4 + SERPINA12 + LowLevel |
| 1285 | CAD ~ Intercept.High + Sex + Age + corin + RBP4 + SERPINA12 + LowLevel |
| 1286 | CAD ~ Intercept.High + Sex + Age + corin + RBP4 + SERPINA12 + LowLevel |
| 1287 | CAD ~ Intercept.High + Sex + Age + corin + RBP4 + LowLevel |
| 1288 | CAD ~ Intercept.High + Sex + Age + corin + RBP4 + LowLevel |
| 1289 | CAD ~ Intercept.High + Sex + Age + corin + RBP4 + LowLevel |
| 1290 | CAD ~ Intercept.High + Sex + Age + corin + RBP4 + LowLevel |
| 1291 | CAD ~ Intercept.High + Sex + Age + corin + RBP4 + LowLevel |
| 1292 | CAD ~ Intercept.High + Sex + Age + corin + RBP4 + LowLevel |
| 1293 | CAD ~ Intercept.High + Sex + Age + corin + RBP4 + LowLevel |
| 1294 | CAD ~ Intercept.High + Sex + Age + corin + RBP4 + LowLevel |
| 1295 | CAD ~ Intercept.High + Sex + Age + corin + RBP4 + LowLevel |
| 1296 | CAD ~ Intercept.High + Sex + Age + corin + RBP4 + LowLevel |
| 1297 | CAD ~ Intercept.High + Sex + Age + corin + SERPINA12 + LowLevel |
| 1298 | CAD ~ Intercept.High + Sex + Age + corin + SERPINA12 + LowLevel |
| 1299 | CAD ~ Intercept.High + Sex + Age + corin + SERPINA12 + LowLevel |
| 1300 | CAD ~ Intercept.High + Sex + Age + corin + SERPINA12 + LowLevel |
| 1301 | CAD ~ Intercept.High + Sex + Age + corin + SERPINA12 + LowLevel |
| 1302 | CAD ~ Intercept.High + Sex + Age + corin + SERPINA12 + LowLevel |
| 1303 | CAD ~ Intercept.High + Sex + Age + corin + SERPINA12 + LowLevel |
| 1304 | CAD ~ Intercept.High + Sex + Age + corin + SERPINA12 + LowLevel |
| 1305 | CAD ~ Intercept.High + Sex + Age + corin + SERPINA12 + LowLevel |
| 1306 | CAD ~ Intercept.High + Sex + Age + corin + SERPINA12 + LowLevel |
| 1307 | CAD ~ Intercept.High + Sex + Age + corin + LowLevel |
| 1308 | CAD ~ Intercept.High + Sex + Age + corin + LowLevel |
| 1309 | CAD ~ Intercept.High + Sex + Age + corin + LowLevel |
| 1310 | CAD ~ Intercept.High + Sex + Age + corin + LowLevel |
| 1311 | CAD ~ Intercept.High + Sex + Age + corin + LowLevel |
| 1312 | CAD ~ Intercept.High + Sex + Age + HSP70 + RBP4 + SERPINA12 + LowLevel |
| 1313 | CAD ~ Intercept.High + Sex + Age + HSP70 + RBP4 + SERPINA12 + LowLevel |
| 1314 | CAD ~ Intercept.High + Sex + Age + HSP70 + RBP4 + SERPINA12 + LowLevel |
| 1315 | CAD ~ Intercept.High + Sex + Age + HSP70 + RBP4 + SERPINA12 + LowLevel |
| 1316 | CAD ~ Intercept.High + Sex + Age + HSP70 + RBP4 + SERPINA12 + LowLevel |
| 1317 | CAD ~ Intercept.High + Sex + Age + HSP70 + RBP4 + SERPINA12 + LowLevel |
| 1318 | CAD ~ Intercept.High + Sex + Age + HSP70 + RBP4 + SERPINA12 + LowLevel |
| 1319 | CAD ~ Intercept.High + Sex + Age + HSP70 + RBP4 + SERPINA12 + LowLevel |
| 1320 | CAD ~ Intercept.High + Sex + Age + HSP70 + RBP4 + SERPINA12 + LowLevel |
| 1321 | CAD ~ Intercept.High + Sex + Age + HSP70 + RBP4 + SERPINA12 + LowLevel |
| 1322 | CAD ~ Intercept.High + Sex + Age + HSP70 + RBP4 + LowLevel |
| 1323 | CAD ~ Intercept.High + Sex + Age + HSP70 + RBP4 + LowLevel |
| 1324 | CAD ~ Intercept.High + Sex + Age + HSP70 + RBP4 + LowLevel |
| 1325 | CAD ~ Intercept.High + Sex + Age + HSP70 + RBP4 + LowLevel |
| 1326 | CAD ~ Intercept.High + Sex + Age + HSP70 + RBP4 + LowLevel |
| 1327 | CAD ~ Intercept.High + Sex + Age + HSP70 + RBP4 + LowLevel |
| 1328 | CAD ~ Intercept.High + Sex + Age + HSP70 + RBP4 + LowLevel |
| 1329 | CAD ~ Intercept.High + Sex + Age + HSP70 + RBP4 + LowLevel |
| 1330 | CAD ~ Intercept.High + Sex + Age + HSP70 + RBP4 + LowLevel |
| 1331 | CAD ~ Intercept.High + Sex + Age + HSP70 + RBP4 + LowLevel |
| 1332 | CAD ~ Intercept.High + Sex + Age + HSP70 + SERPINA12 + LowLevel |
| 1333 | CAD ~ Intercept.High + Sex + Age + HSP70 + SERPINA12 + LowLevel |
| 1334 | CAD ~ Intercept.High + Sex + Age + HSP70 + SERPINA12 + LowLevel |
| 1335 | CAD ~ Intercept.High + Sex + Age + HSP70 + SERPINA12 + LowLevel |
| 1336 | CAD ~ Intercept.High + Sex + Age + HSP70 + SERPINA12 + LowLevel |
| 1337 | CAD ~ Intercept.High + Sex + Age + HSP70 + SERPINA12 + LowLevel |
| 1338 | CAD ~ Intercept.High + Sex + Age + HSP70 + SERPINA12 + LowLevel |
| 1339 | CAD ~ Intercept.High + Sex + Age + HSP70 + SERPINA12 + LowLevel |
| 1340 | CAD ~ Intercept.High + Sex + Age + HSP70 + SERPINA12 + LowLevel |
| 1341 | CAD ~ Intercept.High + Sex + Age + HSP70 + SERPINA12 + LowLevel |
| 1342 | CAD ~ Intercept.High + Sex + Age + HSP70 + LowLevel |
| 1343 | CAD ~ Intercept.High + Sex + Age + HSP70 + LowLevel |
| 1344 | CAD ~ Intercept.High + Sex + Age + HSP70 + LowLevel |
| 1345 | CAD ~ Intercept.High + Sex + Age + HSP70 + LowLevel |
| 1346 | CAD ~ Intercept.High + Sex + Age + HSP70 + LowLevel |
| 1347 | CAD ~ Intercept.High + Sex + Age + RBP4 + SERPINA12 + LowLevel |
| 1348 | CAD ~ Intercept.High + Sex + Age + RBP4 + SERPINA12 + LowLevel |
| 1349 | CAD ~ Intercept.High + Sex + Age + RBP4 + SERPINA12 + LowLevel |
| 1350 | CAD ~ Intercept.High + Sex + Age + RBP4 + SERPINA12 + LowLevel |
| 1351 | CAD ~ Intercept.High + Sex + Age + RBP4 + SERPINA12 + LowLevel |
| 1352 | CAD ~ Intercept.High + Sex + Age + RBP4 + SERPINA12 + LowLevel |
| 1353 | CAD ~ Intercept.High + Sex + Age + RBP4 + SERPINA12 + LowLevel |
| 1354 | CAD ~ Intercept.High + Sex + Age + RBP4 + SERPINA12 + LowLevel |
| 1355 | CAD ~ Intercept.High + Sex + Age + RBP4 + SERPINA12 + LowLevel |
| 1356 | CAD ~ Intercept.High + Sex + Age + RBP4 + SERPINA12 + LowLevel |
| 1357 | CAD ~ Intercept.High + Sex + Age + RBP4 + LowLevel |
| 1358 | CAD ~ Intercept.High + Sex + Age + RBP4 + LowLevel |
| 1359 | CAD ~ Intercept.High + Sex + Age + RBP4 + LowLevel |
| 1360 | CAD ~ Intercept.High + Sex + Age + RBP4 + LowLevel |
| 1361 | CAD ~ Intercept.High + Sex + Age + RBP4 + LowLevel |
| 1362 | CAD ~ Intercept.High + Sex + Age + SERPINA12 + LowLevel |
| 1363 | CAD ~ Intercept.High + Sex + Age + SERPINA12 + LowLevel |
| 1364 | CAD ~ Intercept.High + Sex + Age + SERPINA12 + LowLevel |
| 1365 | CAD ~ Intercept.High + Sex + Age + SERPINA12 + LowLevel |
| 1366 | CAD ~ Intercept.High + Sex + Age + SERPINA12 + LowLevel |
| 1367 | CAD ~ Intercept.High + Sex + Age + LowLevel |
| 1368 | CAD ~ Intercept.High + Sex + APOB + corin + HSP70 + RBP4 + SERPINA12 + LowLevel |
| 1369 | CAD ~ Intercept.High + Sex + APOB + corin + HSP70 + RBP4 + SERPINA12 + LowLevel |
| 1370 | CAD ~ Intercept.High + Sex + APOB + corin + HSP70 + RBP4 + SERPINA12 + LowLevel |
| 1371 | CAD ~ Intercept.High + Sex + APOB + corin + HSP70 + RBP4 + SERPINA12 + LowLevel |
| 1372 | CAD ~ Intercept.High + Sex + APOB + corin + HSP70 + RBP4 + SERPINA12 + LowLevel |
| 1373 | CAD ~ Intercept.High + Sex + APOB + corin + HSP70 + RBP4 + LowLevel |
| 1374 | CAD ~ Intercept.High + Sex + APOB + corin + HSP70 + RBP4 + LowLevel |
| 1375 | CAD ~ Intercept.High + Sex + APOB + corin + HSP70 + RBP4 + LowLevel |
| 1376 | CAD ~ Intercept.High + Sex + APOB + corin + HSP70 + RBP4 + LowLevel |
| 1377 | CAD ~ Intercept.High + Sex + APOB + corin + HSP70 + RBP4 + LowLevel |
| 1378 | CAD ~ Intercept.High + Sex + APOB + corin + HSP70 + RBP4 + LowLevel |
| 1379 | CAD ~ Intercept.High + Sex + APOB + corin + HSP70 + RBP4 + LowLevel |
| 1380 | CAD ~ Intercept.High + Sex + APOB + corin + HSP70 + RBP4 + LowLevel |
| 1381 | CAD ~ Intercept.High + Sex + APOB + corin + HSP70 + RBP4 + LowLevel |
| 1382 | CAD ~ Intercept.High + Sex + APOB + corin + HSP70 + RBP4 + LowLevel |
| 1383 | CAD ~ Intercept.High + Sex + APOB + corin + HSP70 + SERPINA12 + LowLevel |
| 1384 | CAD ~ Intercept.High + Sex + APOB + corin + HSP70 + SERPINA12 + LowLevel |
| 1385 | CAD ~ Intercept.High + Sex + APOB + corin + HSP70 + SERPINA12 + LowLevel |
| 1386 | CAD ~ Intercept.High + Sex + APOB + corin + HSP70 + SERPINA12 + LowLevel |
| 1387 | CAD ~ Intercept.High + Sex + APOB + corin + HSP70 + SERPINA12 + LowLevel |
| 1388 | CAD ~ Intercept.High + Sex + APOB + corin + HSP70 + SERPINA12 + LowLevel |
| 1389 | CAD ~ Intercept.High + Sex + APOB + corin + HSP70 + SERPINA12 + LowLevel |
| 1390 | CAD ~ Intercept.High + Sex + APOB + corin + HSP70 + SERPINA12 + LowLevel |
| 1391 | CAD ~ Intercept.High + Sex + APOB + corin + HSP70 + SERPINA12 + LowLevel |
| 1392 | CAD ~ Intercept.High + Sex + APOB + corin + HSP70 + SERPINA12 + LowLevel |
| 1393 | CAD ~ Intercept.High + Sex + APOB + corin + HSP70 + LowLevel |
| 1394 | CAD ~ Intercept.High + Sex + APOB + corin + HSP70 + LowLevel |
| 1395 | CAD ~ Intercept.High + Sex + APOB + corin + HSP70 + LowLevel |
| 1396 | CAD ~ Intercept.High + Sex + APOB + corin + HSP70 + LowLevel |
| 1397 | CAD ~ Intercept.High + Sex + APOB + corin + HSP70 + LowLevel |
| 1398 | CAD ~ Intercept.High + Sex + APOB + corin + HSP70 + LowLevel |
| 1399 | CAD ~ Intercept.High + Sex + APOB + corin + HSP70 + LowLevel |
| 1400 | CAD ~ Intercept.High + Sex + APOB + corin + HSP70 + LowLevel |
| 1401 | CAD ~ Intercept.High + Sex + APOB + corin + HSP70 + LowLevel |
| 1402 | CAD ~ Intercept.High + Sex + APOB + corin + HSP70 + LowLevel |
| 1403 | CAD ~ Intercept.High + Sex + APOB + corin + RBP4 + SERPINA12 + LowLevel |
| 1404 | CAD ~ Intercept.High + Sex + APOB + corin + RBP4 + SERPINA12 + LowLevel |
| 1405 | CAD ~ Intercept.High + Sex + APOB + corin + RBP4 + SERPINA12 + LowLevel |
| 1406 | CAD ~ Intercept.High + Sex + APOB + corin + RBP4 + SERPINA12 + LowLevel |
| 1407 | CAD ~ Intercept.High + Sex + APOB + corin + RBP4 + SERPINA12 + LowLevel |
| 1408 | CAD ~ Intercept.High + Sex + APOB + corin + RBP4 + SERPINA12 + LowLevel |
| 1409 | CAD ~ Intercept.High + Sex + APOB + corin + RBP4 + SERPINA12 + LowLevel |
| 1410 | CAD ~ Intercept.High + Sex + APOB + corin + RBP4 + SERPINA12 + LowLevel |
| 1411 | CAD ~ Intercept.High + Sex + APOB + corin + RBP4 + SERPINA12 + LowLevel |
| 1412 | CAD ~ Intercept.High + Sex + APOB + corin + RBP4 + SERPINA12 + LowLevel |
| 1413 | CAD ~ Intercept.High + Sex + APOB + corin + RBP4 + LowLevel |
| 1414 | CAD ~ Intercept.High + Sex + APOB + corin + RBP4 + LowLevel |
| 1415 | CAD ~ Intercept.High + Sex + APOB + corin + RBP4 + LowLevel |
| 1416 | CAD ~ Intercept.High + Sex + APOB + corin + RBP4 + LowLevel |
| 1417 | CAD ~ Intercept.High + Sex + APOB + corin + RBP4 + LowLevel |
| 1418 | CAD ~ Intercept.High + Sex + APOB + corin + RBP4 + LowLevel |
| 1419 | CAD ~ Intercept.High + Sex + APOB + corin + RBP4 + LowLevel |
| 1420 | CAD ~ Intercept.High + Sex + APOB + corin + RBP4 + LowLevel |
| 1421 | CAD ~ Intercept.High + Sex + APOB + corin + RBP4 + LowLevel |
| 1422 | CAD ~ Intercept.High + Sex + APOB + corin + RBP4 + LowLevel |
| 1423 | CAD ~ Intercept.High + Sex + APOB + corin + SERPINA12 + LowLevel |
| 1424 | CAD ~ Intercept.High + Sex + APOB + corin + SERPINA12 + LowLevel |
| 1425 | CAD ~ Intercept.High + Sex + APOB + corin + SERPINA12 + LowLevel |
| 1426 | CAD ~ Intercept.High + Sex + APOB + corin + SERPINA12 + LowLevel |
| 1427 | CAD ~ Intercept.High + Sex + APOB + corin + SERPINA12 + LowLevel |
| 1428 | CAD ~ Intercept.High + Sex + APOB + corin + SERPINA12 + LowLevel |
| 1429 | CAD ~ Intercept.High + Sex + APOB + corin + SERPINA12 + LowLevel |
| 1430 | CAD ~ Intercept.High + Sex + APOB + corin + SERPINA12 + LowLevel |
| 1431 | CAD ~ Intercept.High + Sex + APOB + corin + SERPINA12 + LowLevel |
| 1432 | CAD ~ Intercept.High + Sex + APOB + corin + SERPINA12 + LowLevel |
| 1433 | CAD ~ Intercept.High + Sex + APOB + corin + LowLevel |
| 1434 | CAD ~ Intercept.High + Sex + APOB + corin + LowLevel |
| 1435 | CAD ~ Intercept.High + Sex + APOB + corin + LowLevel |
| 1436 | CAD ~ Intercept.High + Sex + APOB + corin + LowLevel |
| 1437 | CAD ~ Intercept.High + Sex + APOB + corin + LowLevel |
| 1438 | CAD ~ Intercept.High + Sex + APOB + HSP70 + RBP4 + SERPINA12 + LowLevel |
| 1439 | CAD ~ Intercept.High + Sex + APOB + HSP70 + RBP4 + SERPINA12 + LowLevel |
| 1440 | CAD ~ Intercept.High + Sex + APOB + HSP70 + RBP4 + SERPINA12 + LowLevel |
| 1441 | CAD ~ Intercept.High + Sex + APOB + HSP70 + RBP4 + SERPINA12 + LowLevel |
| 1442 | CAD ~ Intercept.High + Sex + APOB + HSP70 + RBP4 + SERPINA12 + LowLevel |
| 1443 | CAD ~ Intercept.High + Sex + APOB + HSP70 + RBP4 + SERPINA12 + LowLevel |
| 1444 | CAD ~ Intercept.High + Sex + APOB + HSP70 + RBP4 + SERPINA12 + LowLevel |
| 1445 | CAD ~ Intercept.High + Sex + APOB + HSP70 + RBP4 + SERPINA12 + LowLevel |
| 1446 | CAD ~ Intercept.High + Sex + APOB + HSP70 + RBP4 + SERPINA12 + LowLevel |
| 1447 | CAD ~ Intercept.High + Sex + APOB + HSP70 + RBP4 + SERPINA12 + LowLevel |
| 1448 | CAD ~ Intercept.High + Sex + APOB + HSP70 + RBP4 + LowLevel |
| 1449 | CAD ~ Intercept.High + Sex + APOB + HSP70 + RBP4 + LowLevel |
| 1450 | CAD ~ Intercept.High + Sex + APOB + HSP70 + RBP4 + LowLevel |
| 1451 | CAD ~ Intercept.High + Sex + APOB + HSP70 + RBP4 + LowLevel |
| 1452 | CAD ~ Intercept.High + Sex + APOB + HSP70 + RBP4 + LowLevel |
| 1453 | CAD ~ Intercept.High + Sex + APOB + HSP70 + RBP4 + LowLevel |
| 1454 | CAD ~ Intercept.High + Sex + APOB + HSP70 + RBP4 + LowLevel |
| 1455 | CAD ~ Intercept.High + Sex + APOB + HSP70 + RBP4 + LowLevel |
| 1456 | CAD ~ Intercept.High + Sex + APOB + HSP70 + RBP4 + LowLevel |
| 1457 | CAD ~ Intercept.High + Sex + APOB + HSP70 + RBP4 + LowLevel |
| 1458 | CAD ~ Intercept.High + Sex + APOB + HSP70 + SERPINA12 + LowLevel |
| 1459 | CAD ~ Intercept.High + Sex + APOB + HSP70 + SERPINA12 + LowLevel |
| 1460 | CAD ~ Intercept.High + Sex + APOB + HSP70 + SERPINA12 + LowLevel |
| 1461 | CAD ~ Intercept.High + Sex + APOB + HSP70 + SERPINA12 + LowLevel |
| 1462 | CAD ~ Intercept.High + Sex + APOB + HSP70 + SERPINA12 + LowLevel |
| 1463 | CAD ~ Intercept.High + Sex + APOB + HSP70 + SERPINA12 + LowLevel |
| 1464 | CAD ~ Intercept.High + Sex + APOB + HSP70 + SERPINA12 + LowLevel |
| 1465 | CAD ~ Intercept.High + Sex + APOB + HSP70 + SERPINA12 + LowLevel |
| 1466 | CAD ~ Intercept.High + Sex + APOB + HSP70 + SERPINA12 + LowLevel |
| 1467 | CAD ~ Intercept.High + Sex + APOB + HSP70 + SERPINA12 + LowLevel |
| 1468 | CAD ~ Intercept.High + Sex + APOB + HSP70 + LowLevel |
| 1469 | CAD ~ Intercept.High + Sex + APOB + HSP70 + LowLevel |
| 1470 | CAD ~ Intercept.High + Sex + APOB + HSP70 + LowLevel |
| 1471 | CAD ~ Intercept.High + Sex + APOB + HSP70 + LowLevel |
| 1472 | CAD ~ Intercept.High + Sex + APOB + HSP70 + LowLevel |
| 1473 | CAD ~ Intercept.High + Sex + APOB + RBP4 + SERPINA12 + LowLevel |
| 1474 | CAD ~ Intercept.High + Sex + APOB + RBP4 + SERPINA12 + LowLevel |
| 1475 | CAD ~ Intercept.High + Sex + APOB + RBP4 + SERPINA12 + LowLevel |
| 1476 | CAD ~ Intercept.High + Sex + APOB + RBP4 + SERPINA12 + LowLevel |
| 1477 | CAD ~ Intercept.High + Sex + APOB + RBP4 + SERPINA12 + LowLevel |
| 1478 | CAD ~ Intercept.High + Sex + APOB + RBP4 + SERPINA12 + LowLevel |
| 1479 | CAD ~ Intercept.High + Sex + APOB + RBP4 + SERPINA12 + LowLevel |
| 1480 | CAD ~ Intercept.High + Sex + APOB + RBP4 + SERPINA12 + LowLevel |
| 1481 | CAD ~ Intercept.High + Sex + APOB + RBP4 + SERPINA12 + LowLevel |
| 1482 | CAD ~ Intercept.High + Sex + APOB + RBP4 + SERPINA12 + LowLevel |
| 1483 | CAD ~ Intercept.High + Sex + APOB + RBP4 + LowLevel |
| 1484 | CAD ~ Intercept.High + Sex + APOB + RBP4 + LowLevel |
| 1485 | CAD ~ Intercept.High + Sex + APOB + RBP4 + LowLevel |
| 1486 | CAD ~ Intercept.High + Sex + APOB + RBP4 + LowLevel |
| 1487 | CAD ~ Intercept.High + Sex + APOB + RBP4 + LowLevel |
| 1488 | CAD ~ Intercept.High + Sex + APOB + SERPINA12 + LowLevel |
| 1489 | CAD ~ Intercept.High + Sex + APOB + SERPINA12 + LowLevel |
| 1490 | CAD ~ Intercept.High + Sex + APOB + SERPINA12 + LowLevel |
| 1491 | CAD ~ Intercept.High + Sex + APOB + SERPINA12 + LowLevel |
| 1492 | CAD ~ Intercept.High + Sex + APOB + SERPINA12 + LowLevel |
| 1493 | CAD ~ Intercept.High + Sex + APOB + LowLevel |
| 1494 | CAD ~ Intercept.High + Sex + corin + HSP70 + RBP4 + SERPINA12 + LowLevel |
| 1495 | CAD ~ Intercept.High + Sex + corin + HSP70 + RBP4 + SERPINA12 + LowLevel |
| 1496 | CAD ~ Intercept.High + Sex + corin + HSP70 + RBP4 + SERPINA12 + LowLevel |
| 1497 | CAD ~ Intercept.High + Sex + corin + HSP70 + RBP4 + SERPINA12 + LowLevel |
| 1498 | CAD ~ Intercept.High + Sex + corin + HSP70 + RBP4 + SERPINA12 + LowLevel |
| 1499 | CAD ~ Intercept.High + Sex + corin + HSP70 + RBP4 + SERPINA12 + LowLevel |
| 1500 | CAD ~ Intercept.High + Sex + corin + HSP70 + RBP4 + SERPINA12 + LowLevel |
| 1501 | CAD ~ Intercept.High + Sex + corin + HSP70 + RBP4 + SERPINA12 + LowLevel |
| 1502 | CAD ~ Intercept.High + Sex + corin + HSP70 + RBP4 + SERPINA12 + LowLevel |
| 1503 | CAD ~ Intercept.High + Sex + corin + HSP70 + RBP4 + SERPINA12 + LowLevel |
| 1504 | CAD ~ Intercept.High + Sex + corin + HSP70 + RBP4 + LowLevel |
| 1505 | CAD ~ Intercept.High + Sex + corin + HSP70 + RBP4 + LowLevel |
| 1506 | CAD ~ Intercept.High + Sex + corin + HSP70 + RBP4 + LowLevel |
| 1507 | CAD ~ Intercept.High + Sex + corin + HSP70 + RBP4 + LowLevel |
| 1508 | CAD ~ Intercept.High + Sex + corin + HSP70 + RBP4 + LowLevel |
| 1509 | CAD ~ Intercept.High + Sex + corin + HSP70 + RBP4 + LowLevel |
| 1510 | CAD ~ Intercept.High + Sex + corin + HSP70 + RBP4 + LowLevel |
| 1511 | CAD ~ Intercept.High + Sex + corin + HSP70 + RBP4 + LowLevel |
| 1512 | CAD ~ Intercept.High + Sex + corin + HSP70 + RBP4 + LowLevel |
| 1513 | CAD ~ Intercept.High + Sex + corin + HSP70 + RBP4 + LowLevel |
| 1514 | CAD ~ Intercept.High + Sex + corin + HSP70 + SERPINA12 + LowLevel |
| 1515 | CAD ~ Intercept.High + Sex + corin + HSP70 + SERPINA12 + LowLevel |
| 1516 | CAD ~ Intercept.High + Sex + corin + HSP70 + SERPINA12 + LowLevel |
| 1517 | CAD ~ Intercept.High + Sex + corin + HSP70 + SERPINA12 + LowLevel |
| 1518 | CAD ~ Intercept.High + Sex + corin + HSP70 + SERPINA12 + LowLevel |
| 1519 | CAD ~ Intercept.High + Sex + corin + HSP70 + SERPINA12 + LowLevel |
| 1520 | CAD ~ Intercept.High + Sex + corin + HSP70 + SERPINA12 + LowLevel |
| 1521 | CAD ~ Intercept.High + Sex + corin + HSP70 + SERPINA12 + LowLevel |
| 1522 | CAD ~ Intercept.High + Sex + corin + HSP70 + SERPINA12 + LowLevel |
| 1523 | CAD ~ Intercept.High + Sex + corin + HSP70 + SERPINA12 + LowLevel |
| 1524 | CAD ~ Intercept.High + Sex + corin + HSP70 + LowLevel |
| 1525 | CAD ~ Intercept.High + Sex + corin + HSP70 + LowLevel |
| 1526 | CAD ~ Intercept.High + Sex + corin + HSP70 + LowLevel |
| 1527 | CAD ~ Intercept.High + Sex + corin + HSP70 + LowLevel |
| 1528 | CAD ~ Intercept.High + Sex + corin + HSP70 + LowLevel |
| 1529 | CAD ~ Intercept.High + Sex + corin + RBP4 + SERPINA12 + LowLevel |
| 1530 | CAD ~ Intercept.High + Sex + corin + RBP4 + SERPINA12 + LowLevel |
| 1531 | CAD ~ Intercept.High + Sex + corin + RBP4 + SERPINA12 + LowLevel |
| 1532 | CAD ~ Intercept.High + Sex + corin + RBP4 + SERPINA12 + LowLevel |
| 1533 | CAD ~ Intercept.High + Sex + corin + RBP4 + SERPINA12 + LowLevel |
| 1534 | CAD ~ Intercept.High + Sex + corin + RBP4 + SERPINA12 + LowLevel |
| 1535 | CAD ~ Intercept.High + Sex + corin + RBP4 + SERPINA12 + LowLevel |
| 1536 | CAD ~ Intercept.High + Sex + corin + RBP4 + SERPINA12 + LowLevel |
| 1537 | CAD ~ Intercept.High + Sex + corin + RBP4 + SERPINA12 + LowLevel |
| 1538 | CAD ~ Intercept.High + Sex + corin + RBP4 + SERPINA12 + LowLevel |
| 1539 | CAD ~ Intercept.High + Sex + corin + RBP4 + LowLevel |
| 1540 | CAD ~ Intercept.High + Sex + corin + RBP4 + LowLevel |
| 1541 | CAD ~ Intercept.High + Sex + corin + RBP4 + LowLevel |
| 1542 | CAD ~ Intercept.High + Sex + corin + RBP4 + LowLevel |
| 1543 | CAD ~ Intercept.High + Sex + corin + RBP4 + LowLevel |
| 1544 | CAD ~ Intercept.High + Sex + corin + SERPINA12 + LowLevel |
| 1545 | CAD ~ Intercept.High + Sex + corin + SERPINA12 + LowLevel |
| 1546 | CAD ~ Intercept.High + Sex + corin + SERPINA12 + LowLevel |
| 1547 | CAD ~ Intercept.High + Sex + corin + SERPINA12 + LowLevel |
| 1548 | CAD ~ Intercept.High + Sex + corin + SERPINA12 + LowLevel |
| 1549 | CAD ~ Intercept.High + Sex + corin + LowLevel |
| 1550 | CAD ~ Intercept.High + Sex + HSP70 + RBP4 + SERPINA12 + LowLevel |
| 1551 | CAD ~ Intercept.High + Sex + HSP70 + RBP4 + SERPINA12 + LowLevel |
| 1552 | CAD ~ Intercept.High + Sex + HSP70 + RBP4 + SERPINA12 + LowLevel |
| 1553 | CAD ~ Intercept.High + Sex + HSP70 + RBP4 + SERPINA12 + LowLevel |
| 1554 | CAD ~ Intercept.High + Sex + HSP70 + RBP4 + SERPINA12 + LowLevel |
| 1555 | CAD ~ Intercept.High + Sex + HSP70 + RBP4 + SERPINA12 + LowLevel |
| 1556 | CAD ~ Intercept.High + Sex + HSP70 + RBP4 + SERPINA12 + LowLevel |
| 1557 | CAD ~ Intercept.High + Sex + HSP70 + RBP4 + SERPINA12 + LowLevel |
| 1558 | CAD ~ Intercept.High + Sex + HSP70 + RBP4 + SERPINA12 + LowLevel |
| 1559 | CAD ~ Intercept.High + Sex + HSP70 + RBP4 + SERPINA12 + LowLevel |
| 1560 | CAD ~ Intercept.High + Sex + HSP70 + RBP4 + LowLevel |
| 1561 | CAD ~ Intercept.High + Sex + HSP70 + RBP4 + LowLevel |
| 1562 | CAD ~ Intercept.High + Sex + HSP70 + RBP4 + LowLevel |
| 1563 | CAD ~ Intercept.High + Sex + HSP70 + RBP4 + LowLevel |
| 1564 | CAD ~ Intercept.High + Sex + HSP70 + RBP4 + LowLevel |
| 1565 | CAD ~ Intercept.High + Sex + HSP70 + SERPINA12 + LowLevel |
| 1566 | CAD ~ Intercept.High + Sex + HSP70 + SERPINA12 + LowLevel |
| 1567 | CAD ~ Intercept.High + Sex + HSP70 + SERPINA12 + LowLevel |
| 1568 | CAD ~ Intercept.High + Sex + HSP70 + SERPINA12 + LowLevel |
| 1569 | CAD ~ Intercept.High + Sex + HSP70 + SERPINA12 + LowLevel |
| 1570 | CAD ~ Intercept.High + Sex + HSP70 + LowLevel |
| 1571 | CAD ~ Intercept.High + Sex + RBP4 + SERPINA12 + LowLevel |
| 1572 | CAD ~ Intercept.High + Sex + RBP4 + SERPINA12 + LowLevel |
| 1573 | CAD ~ Intercept.High + Sex + RBP4 + SERPINA12 + LowLevel |
| 1574 | CAD ~ Intercept.High + Sex + RBP4 + SERPINA12 + LowLevel |
| 1575 | CAD ~ Intercept.High + Sex + RBP4 + SERPINA12 + LowLevel |
| 1576 | CAD ~ Intercept.High + Sex + RBP4 + LowLevel |
| 1577 | CAD ~ Intercept.High + Sex + SERPINA12 + LowLevel |
| 1578 | CAD ~ Intercept.High + HSP70 + RBP4 + SERPINA12 + LowLevel |
| 1579 | CAD ~ Intercept.High + HSP70 + RBP4 + SERPINA12 + LowLevel |
| 1580 | CAD ~ Intercept.High + HSP70 + RBP4 + SERPINA12 + LowLevel |
| 1581 | CAD ~ Intercept.High + HSP70 + RBP4 + SERPINA12 + LowLevel |
| 1582 | CAD ~ Intercept.High + HSP70 + RBP4 + SERPINA12 + LowLevel |
| 1583 | CAD ~ Intercept.High + HSP70 + RBP4 + LowLevel |
| 1584 | CAD ~ Intercept.High + HSP70 + SERPINA12 + LowLevel |
| 1585 | CAD ~ Intercept.High + RBP4 + SERPINA12 + LowLevel |
| 1586 | CAD ~ Intercept.High + Age + APOB + corin + HSP70 + RBP4 + LowLevel |
| 1587 | CAD ~ Intercept.High + Age + APOB + corin + HSP70 + RBP4 + LowLevel |
| 1588 | CAD ~ Intercept.High + Age + APOB + corin + HSP70 + RBP4 + LowLevel |
| 1589 | CAD ~ Intercept.High + Age + APOB + corin + HSP70 + RBP4 + LowLevel |
| 1590 | CAD ~ Intercept.High + Age + APOB + corin + HSP70 + RBP4 + LowLevel |
| 1591 | CAD ~ Intercept.High + Age + APOB + corin + HSP70 + SERPINA12 + LowLevel |
| 1592 | CAD ~ Intercept.High + Age + APOB + corin + HSP70 + SERPINA12 + LowLevel |
| 1593 | CAD ~ Intercept.High + Age + APOB + corin + HSP70 + SERPINA12 + LowLevel |
| 1594 | CAD ~ Intercept.High + Age + APOB + corin + HSP70 + SERPINA12 + LowLevel |
| 1595 | CAD ~ Intercept.High + Age + APOB + corin + HSP70 + SERPINA12 + LowLevel |
| 1596 | CAD ~ Intercept.High + Age + APOB + corin + HSP70 + LowLevel |
| 1597 | CAD ~ Intercept.High + Age + APOB + corin + HSP70 + LowLevel |
| 1598 | CAD ~ Intercept.High + Age + APOB + corin + HSP70 + LowLevel |
| 1599 | CAD ~ Intercept.High + Age + APOB + corin + HSP70 + LowLevel |
| 1600 | CAD ~ Intercept.High + Age + APOB + corin + HSP70 + LowLevel |
| 1601 | CAD ~ Intercept.High + Age + APOB + corin + HSP70 + LowLevel |
| 1602 | CAD ~ Intercept.High + Age + APOB + corin + HSP70 + LowLevel |
| 1603 | CAD ~ Intercept.High + Age + APOB + corin + HSP70 + LowLevel |
| 1604 | CAD ~ Intercept.High + Age + APOB + corin + HSP70 + LowLevel |
| 1605 | CAD ~ Intercept.High + Age + APOB + corin + HSP70 + LowLevel |
| 1606 | CAD ~ Intercept.High + Age + APOB + corin + RBP4 + SERPINA12 + LowLevel |
| 1607 | CAD ~ Intercept.High + Age + APOB + corin + RBP4 + SERPINA12 + LowLevel |
| 1608 | CAD ~ Intercept.High + Age + APOB + corin + RBP4 + SERPINA12 + LowLevel |
| 1609 | CAD ~ Intercept.High + Age + APOB + corin + RBP4 + SERPINA12 + LowLevel |
| 1610 | CAD ~ Intercept.High + Age + APOB + corin + RBP4 + SERPINA12 + LowLevel |
| 1611 | CAD ~ Intercept.High + Age + APOB + corin + RBP4 + LowLevel |
| 1612 | CAD ~ Intercept.High + Age + APOB + corin + RBP4 + LowLevel |
| 1613 | CAD ~ Intercept.High + Age + APOB + corin + RBP4 + LowLevel |
| 1614 | CAD ~ Intercept.High + Age + APOB + corin + RBP4 + LowLevel |
| 1615 | CAD ~ Intercept.High + Age + APOB + corin + RBP4 + LowLevel |
| 1616 | CAD ~ Intercept.High + Age + APOB + corin + RBP4 + LowLevel |
| 1617 | CAD ~ Intercept.High + Age + APOB + corin + RBP4 + LowLevel |
| 1618 | CAD ~ Intercept.High + Age + APOB + corin + RBP4 + LowLevel |
| 1619 | CAD ~ Intercept.High + Age + APOB + corin + RBP4 + LowLevel |
| 1620 | CAD ~ Intercept.High + Age + APOB + corin + RBP4 + LowLevel |
| 1621 | CAD ~ Intercept.High + Age + APOB + corin + SERPINA12 + LowLevel |
| 1622 | CAD ~ Intercept.High + Age + APOB + corin + SERPINA12 + LowLevel |
| 1623 | CAD ~ Intercept.High + Age + APOB + corin + SERPINA12 + LowLevel |
| 1624 | CAD ~ Intercept.High + Age + APOB + corin + SERPINA12 + LowLevel |
| 1625 | CAD ~ Intercept.High + Age + APOB + corin + SERPINA12 + LowLevel |
| 1626 | CAD ~ Intercept.High + Age + APOB + corin + SERPINA12 + LowLevel |
| 1627 | CAD ~ Intercept.High + Age + APOB + corin + SERPINA12 + LowLevel |
| 1628 | CAD ~ Intercept.High + Age + APOB + corin + SERPINA12 + LowLevel |
| 1629 | CAD ~ Intercept.High + Age + APOB + corin + SERPINA12 + LowLevel |
| 1630 | CAD ~ Intercept.High + Age + APOB + corin + SERPINA12 + LowLevel |
| 1631 | CAD ~ Intercept.High + Age + APOB + corin + LowLevel |
| 1632 | CAD ~ Intercept.High + Age + APOB + corin + LowLevel |
| 1633 | CAD ~ Intercept.High + Age + APOB + corin + LowLevel |
| 1634 | CAD ~ Intercept.High + Age + APOB + corin + LowLevel |
| 1635 | CAD ~ Intercept.High + Age + APOB + corin + LowLevel |
| 1636 | CAD ~ Intercept.High + Age + APOB + corin + LowLevel |
| 1637 | CAD ~ Intercept.High + Age + APOB + corin + LowLevel |
| 1638 | CAD ~ Intercept.High + Age + APOB + corin + LowLevel |
| 1639 | CAD ~ Intercept.High + Age + APOB + corin + LowLevel |
| 1640 | CAD ~ Intercept.High + Age + APOB + corin + LowLevel |
| 1641 | CAD ~ Intercept.High + Age + APOB + HSP70 + RBP4 + SERPINA12 + LowLevel |
| 1642 | CAD ~ Intercept.High + Age + APOB + HSP70 + RBP4 + SERPINA12 + LowLevel |
| 1643 | CAD ~ Intercept.High + Age + APOB + HSP70 + RBP4 + SERPINA12 + LowLevel |
| 1644 | CAD ~ Intercept.High + Age + APOB + HSP70 + RBP4 + SERPINA12 + LowLevel |
| 1645 | CAD ~ Intercept.High + Age + APOB + HSP70 + RBP4 + SERPINA12 + LowLevel |
| 1646 | CAD ~ Intercept.High + Age + APOB + HSP70 + RBP4 + LowLevel |
| 1647 | CAD ~ Intercept.High + Age + APOB + HSP70 + RBP4 + LowLevel |
| 1648 | CAD ~ Intercept.High + Age + APOB + HSP70 + RBP4 + LowLevel |
| 1649 | CAD ~ Intercept.High + Age + APOB + HSP70 + RBP4 + LowLevel |
| 1650 | CAD ~ Intercept.High + Age + APOB + HSP70 + RBP4 + LowLevel |
| 1651 | CAD ~ Intercept.High + Age + APOB + HSP70 + RBP4 + LowLevel |
| 1652 | CAD ~ Intercept.High + Age + APOB + HSP70 + RBP4 + LowLevel |
| 1653 | CAD ~ Intercept.High + Age + APOB + HSP70 + RBP4 + LowLevel |
| 1654 | CAD ~ Intercept.High + Age + APOB + HSP70 + RBP4 + LowLevel |
| 1655 | CAD ~ Intercept.High + Age + APOB + HSP70 + RBP4 + LowLevel |
| 1656 | CAD ~ Intercept.High + Age + APOB + HSP70 + SERPINA12 + LowLevel |
| 1657 | CAD ~ Intercept.High + Age + APOB + HSP70 + SERPINA12 + LowLevel |
| 1658 | CAD ~ Intercept.High + Age + APOB + HSP70 + SERPINA12 + LowLevel |
| 1659 | CAD ~ Intercept.High + Age + APOB + HSP70 + SERPINA12 + LowLevel |
| 1660 | CAD ~ Intercept.High + Age + APOB + HSP70 + SERPINA12 + LowLevel |
| 1661 | CAD ~ Intercept.High + Age + APOB + HSP70 + SERPINA12 + LowLevel |
| 1662 | CAD ~ Intercept.High + Age + APOB + HSP70 + SERPINA12 + LowLevel |
| 1663 | CAD ~ Intercept.High + Age + APOB + HSP70 + SERPINA12 + LowLevel |
| 1664 | CAD ~ Intercept.High + Age + APOB + HSP70 + SERPINA12 + LowLevel |
| 1665 | CAD ~ Intercept.High + Age + APOB + HSP70 + SERPINA12 + LowLevel |
| 1666 | CAD ~ Intercept.High + Age + APOB + HSP70 + LowLevel |
| 1667 | CAD ~ Intercept.High + Age + APOB + HSP70 + LowLevel |
| 1668 | CAD ~ Intercept.High + Age + APOB + HSP70 + LowLevel |
| 1669 | CAD ~ Intercept.High + Age + APOB + HSP70 + LowLevel |
| 1670 | CAD ~ Intercept.High + Age + APOB + HSP70 + LowLevel |
| 1671 | CAD ~ Intercept.High + Age + APOB + HSP70 + LowLevel |
| 1672 | CAD ~ Intercept.High + Age + APOB + HSP70 + LowLevel |
| 1673 | CAD ~ Intercept.High + Age + APOB + HSP70 + LowLevel |
| 1674 | CAD ~ Intercept.High + Age + APOB + HSP70 + LowLevel |
| 1675 | CAD ~ Intercept.High + Age + APOB + HSP70 + LowLevel |
| 1676 | CAD ~ Intercept.High + Age + APOB + RBP4 + SERPINA12 + LowLevel |
| 1677 | CAD ~ Intercept.High + Age + APOB + RBP4 + SERPINA12 + LowLevel |
| 1678 | CAD ~ Intercept.High + Age + APOB + RBP4 + SERPINA12 + LowLevel |
| 1679 | CAD ~ Intercept.High + Age + APOB + RBP4 + SERPINA12 + LowLevel |
| 1680 | CAD ~ Intercept.High + Age + APOB + RBP4 + SERPINA12 + LowLevel |
| 1681 | CAD ~ Intercept.High + Age + APOB + RBP4 + SERPINA12 + LowLevel |
| 1682 | CAD ~ Intercept.High + Age + APOB + RBP4 + SERPINA12 + LowLevel |
| 1683 | CAD ~ Intercept.High + Age + APOB + RBP4 + SERPINA12 + LowLevel |
| 1684 | CAD ~ Intercept.High + Age + APOB + RBP4 + SERPINA12 + LowLevel |
| 1685 | CAD ~ Intercept.High + Age + APOB + RBP4 + SERPINA12 + LowLevel |
| 1686 | CAD ~ Intercept.High + Age + APOB + RBP4 + LowLevel |
| 1687 | CAD ~ Intercept.High + Age + APOB + RBP4 + LowLevel |
| 1688 | CAD ~ Intercept.High + Age + APOB + RBP4 + LowLevel |
| 1689 | CAD ~ Intercept.High + Age + APOB + RBP4 + LowLevel |
| 1690 | CAD ~ Intercept.High + Age + APOB + RBP4 + LowLevel |
| 1691 | CAD ~ Intercept.High + Age + APOB + RBP4 + LowLevel |
| 1692 | CAD ~ Intercept.High + Age + APOB + RBP4 + LowLevel |
| 1693 | CAD ~ Intercept.High + Age + APOB + RBP4 + LowLevel |
| 1694 | CAD ~ Intercept.High + Age + APOB + RBP4 + LowLevel |
| 1695 | CAD ~ Intercept.High + Age + APOB + RBP4 + LowLevel |
| 1696 | CAD ~ Intercept.High + Age + APOB + SERPINA12 + LowLevel |
| 1697 | CAD ~ Intercept.High + Age + APOB + SERPINA12 + LowLevel |
| 1698 | CAD ~ Intercept.High + Age + APOB + SERPINA12 + LowLevel |
| 1699 | CAD ~ Intercept.High + Age + APOB + SERPINA12 + LowLevel |
| 1700 | CAD ~ Intercept.High + Age + APOB + SERPINA12 + LowLevel |
| 1701 | CAD ~ Intercept.High + Age + APOB + SERPINA12 + LowLevel |
| 1702 | CAD ~ Intercept.High + Age + APOB + SERPINA12 + LowLevel |
| 1703 | CAD ~ Intercept.High + Age + APOB + SERPINA12 + LowLevel |
| 1704 | CAD ~ Intercept.High + Age + APOB + SERPINA12 + LowLevel |
| 1705 | CAD ~ Intercept.High + Age + APOB + SERPINA12 + LowLevel |
| 1706 | CAD ~ Intercept.High + Age + APOB + LowLevel |
| 1707 | CAD ~ Intercept.High + Age + APOB + LowLevel |
| 1708 | CAD ~ Intercept.High + Age + APOB + LowLevel |
| 1709 | CAD ~ Intercept.High + Age + APOB + LowLevel |
| 1710 | CAD ~ Intercept.High + Age + APOB + LowLevel |
| 1711 | CAD ~ Intercept.High + Age + corin + HSP70 + RBP4 + SERPINA12 + LowLevel |
| 1712 | CAD ~ Intercept.High + Age + corin + HSP70 + RBP4 + SERPINA12 + LowLevel |
| 1713 | CAD ~ Intercept.High + Age + corin + HSP70 + RBP4 + SERPINA12 + LowLevel |
| 1714 | CAD ~ Intercept.High + Age + corin + HSP70 + RBP4 + SERPINA12 + LowLevel |
| 1715 | CAD ~ Intercept.High + Age + corin + HSP70 + RBP4 + SERPINA12 + LowLevel |
| 1716 | CAD ~ Intercept.High + Age + corin + HSP70 + RBP4 + LowLevel |
| 1717 | CAD ~ Intercept.High + Age + corin + HSP70 + RBP4 + LowLevel |
| 1718 | CAD ~ Intercept.High + Age + corin + HSP70 + RBP4 + LowLevel |
| 1719 | CAD ~ Intercept.High + Age + corin + HSP70 + RBP4 + LowLevel |
| 1720 | CAD ~ Intercept.High + Age + corin + HSP70 + RBP4 + LowLevel |
| 1721 | CAD ~ Intercept.High + Age + corin + HSP70 + RBP4 + LowLevel |
| 1722 | CAD ~ Intercept.High + Age + corin + HSP70 + RBP4 + LowLevel |
| 1723 | CAD ~ Intercept.High + Age + corin + HSP70 + RBP4 + LowLevel |
| 1724 | CAD ~ Intercept.High + Age + corin + HSP70 + RBP4 + LowLevel |
| 1725 | CAD ~ Intercept.High + Age + corin + HSP70 + RBP4 + LowLevel |
| 1726 | CAD ~ Intercept.High + Age + corin + HSP70 + SERPINA12 + LowLevel |
| 1727 | CAD ~ Intercept.High + Age + corin + HSP70 + SERPINA12 + LowLevel |
| 1728 | CAD ~ Intercept.High + Age + corin + HSP70 + SERPINA12 + LowLevel |
| 1729 | CAD ~ Intercept.High + Age + corin + HSP70 + SERPINA12 + LowLevel |
| 1730 | CAD ~ Intercept.High + Age + corin + HSP70 + SERPINA12 + LowLevel |
| 1731 | CAD ~ Intercept.High + Age + corin + HSP70 + SERPINA12 + LowLevel |
| 1732 | CAD ~ Intercept.High + Age + corin + HSP70 + SERPINA12 + LowLevel |
| 1733 | CAD ~ Intercept.High + Age + corin + HSP70 + SERPINA12 + LowLevel |
| 1734 | CAD ~ Intercept.High + Age + corin + HSP70 + SERPINA12 + LowLevel |
| 1735 | CAD ~ Intercept.High + Age + corin + HSP70 + SERPINA12 + LowLevel |
| 1736 | CAD ~ Intercept.High + Age + corin + HSP70 + LowLevel |
| 1737 | CAD ~ Intercept.High + Age + corin + HSP70 + LowLevel |
| 1738 | CAD ~ Intercept.High + Age + corin + HSP70 + LowLevel |
| 1739 | CAD ~ Intercept.High + Age + corin + HSP70 + LowLevel |
| 1740 | CAD ~ Intercept.High + Age + corin + HSP70 + LowLevel |
| 1741 | CAD ~ Intercept.High + Age + corin + HSP70 + LowLevel |
| 1742 | CAD ~ Intercept.High + Age + corin + HSP70 + LowLevel |
| 1743 | CAD ~ Intercept.High + Age + corin + HSP70 + LowLevel |
| 1744 | CAD ~ Intercept.High + Age + corin + HSP70 + LowLevel |
| 1745 | CAD ~ Intercept.High + Age + corin + HSP70 + LowLevel |
| 1746 | CAD ~ Intercept.High + Age + corin + RBP4 + SERPINA12 + LowLevel |
| 1747 | CAD ~ Intercept.High + Age + corin + RBP4 + SERPINA12 + LowLevel |
| 1748 | CAD ~ Intercept.High + Age + corin + RBP4 + SERPINA12 + LowLevel |
| 1749 | CAD ~ Intercept.High + Age + corin + RBP4 + SERPINA12 + LowLevel |
| 1750 | CAD ~ Intercept.High + Age + corin + RBP4 + SERPINA12 + LowLevel |
| 1751 | CAD ~ Intercept.High + Age + corin + RBP4 + SERPINA12 + LowLevel |
| 1752 | CAD ~ Intercept.High + Age + corin + RBP4 + SERPINA12 + LowLevel |
| 1753 | CAD ~ Intercept.High + Age + corin + RBP4 + SERPINA12 + LowLevel |
| 1754 | CAD ~ Intercept.High + Age + corin + RBP4 + SERPINA12 + LowLevel |
| 1755 | CAD ~ Intercept.High + Age + corin + RBP4 + SERPINA12 + LowLevel |
| 1756 | CAD ~ Intercept.High + Age + corin + RBP4 + LowLevel |
| 1757 | CAD ~ Intercept.High + Age + corin + RBP4 + LowLevel |
| 1758 | CAD ~ Intercept.High + Age + corin + RBP4 + LowLevel |
| 1759 | CAD ~ Intercept.High + Age + corin + RBP4 + LowLevel |
| 1760 | CAD ~ Intercept.High + Age + corin + RBP4 + LowLevel |
| 1761 | CAD ~ Intercept.High + Age + corin + RBP4 + LowLevel |
| 1762 | CAD ~ Intercept.High + Age + corin + RBP4 + LowLevel |
| 1763 | CAD ~ Intercept.High + Age + corin + RBP4 + LowLevel |
| 1764 | CAD ~ Intercept.High + Age + corin + RBP4 + LowLevel |
| 1765 | CAD ~ Intercept.High + Age + corin + RBP4 + LowLevel |
| 1766 | CAD ~ Intercept.High + Age + corin + SERPINA12 + LowLevel |
| 1767 | CAD ~ Intercept.High + Age + corin + SERPINA12 + LowLevel |
| 1768 | CAD ~ Intercept.High + Age + corin + SERPINA12 + LowLevel |
| 1769 | CAD ~ Intercept.High + Age + corin + SERPINA12 + LowLevel |
| 1770 | CAD ~ Intercept.High + Age + corin + SERPINA12 + LowLevel |
| 1771 | CAD ~ Intercept.High + Age + corin + SERPINA12 + LowLevel |
| 1772 | CAD ~ Intercept.High + Age + corin + SERPINA12 + LowLevel |
| 1773 | CAD ~ Intercept.High + Age + corin + SERPINA12 + LowLevel |
| 1774 | CAD ~ Intercept.High + Age + corin + SERPINA12 + LowLevel |
| 1775 | CAD ~ Intercept.High + Age + corin + SERPINA12 + LowLevel |
| 1776 | CAD ~ Intercept.High + Age + corin + LowLevel |
| 1777 | CAD ~ Intercept.High + Age + corin + LowLevel |
| 1778 | CAD ~ Intercept.High + Age + corin + LowLevel |
| 1779 | CAD ~ Intercept.High + Age + corin + LowLevel |
| 1780 | CAD ~ Intercept.High + Age + corin + LowLevel |
| 1781 | CAD ~ Intercept.High + Age + HSP70 + RBP4 + SERPINA12 + LowLevel |
| 1782 | CAD ~ Intercept.High + Age + HSP70 + RBP4 + SERPINA12 + LowLevel |
| 1783 | CAD ~ Intercept.High + Age + HSP70 + RBP4 + SERPINA12 + LowLevel |
| 1784 | CAD ~ Intercept.High + Age + HSP70 + RBP4 + SERPINA12 + LowLevel |
| 1785 | CAD ~ Intercept.High + Age + HSP70 + RBP4 + SERPINA12 + LowLevel |
| 1786 | CAD ~ Intercept.High + Age + HSP70 + RBP4 + SERPINA12 + LowLevel |
| 1787 | CAD ~ Intercept.High + Age + HSP70 + RBP4 + SERPINA12 + LowLevel |
| 1788 | CAD ~ Intercept.High + Age + HSP70 + RBP4 + SERPINA12 + LowLevel |
| 1789 | CAD ~ Intercept.High + Age + HSP70 + RBP4 + SERPINA12 + LowLevel |
| 1790 | CAD ~ Intercept.High + Age + HSP70 + RBP4 + SERPINA12 + LowLevel |
| 1791 | CAD ~ Intercept.High + Age + HSP70 + RBP4 + LowLevel |
| 1792 | CAD ~ Intercept.High + Age + HSP70 + RBP4 + LowLevel |
| 1793 | CAD ~ Intercept.High + Age + HSP70 + RBP4 + LowLevel |
| 1794 | CAD ~ Intercept.High + Age + HSP70 + RBP4 + LowLevel |
| 1795 | CAD ~ Intercept.High + Age + HSP70 + RBP4 + LowLevel |
| 1796 | CAD ~ Intercept.High + Age + HSP70 + RBP4 + LowLevel |
| 1797 | CAD ~ Intercept.High + Age + HSP70 + RBP4 + LowLevel |
| 1798 | CAD ~ Intercept.High + Age + HSP70 + RBP4 + LowLevel |
| 1799 | CAD ~ Intercept.High + Age + HSP70 + RBP4 + LowLevel |
| 1800 | CAD ~ Intercept.High + Age + HSP70 + RBP4 + LowLevel |
| 1801 | CAD ~ Intercept.High + Age + HSP70 + SERPINA12 + LowLevel |
| 1802 | CAD ~ Intercept.High + Age + HSP70 + SERPINA12 + LowLevel |
| 1803 | CAD ~ Intercept.High + Age + HSP70 + SERPINA12 + LowLevel |
| 1804 | CAD ~ Intercept.High + Age + HSP70 + SERPINA12 + LowLevel |
| 1805 | CAD ~ Intercept.High + Age + HSP70 + SERPINA12 + LowLevel |
| 1806 | CAD ~ Intercept.High + Age + HSP70 + SERPINA12 + LowLevel |
| 1807 | CAD ~ Intercept.High + Age + HSP70 + SERPINA12 + LowLevel |
| 1808 | CAD ~ Intercept.High + Age + HSP70 + SERPINA12 + LowLevel |
| 1809 | CAD ~ Intercept.High + Age + HSP70 + SERPINA12 + LowLevel |
| 1810 | CAD ~ Intercept.High + Age + HSP70 + SERPINA12 + LowLevel |
| 1811 | CAD ~ Intercept.High + Age + HSP70 + LowLevel |
| 1812 | CAD ~ Intercept.High + Age + HSP70 + LowLevel |
| 1813 | CAD ~ Intercept.High + Age + HSP70 + LowLevel |
| 1814 | CAD ~ Intercept.High + Age + HSP70 + LowLevel |
| 1815 | CAD ~ Intercept.High + Age + HSP70 + LowLevel |
| 1816 | CAD ~ Intercept.High + Age + RBP4 + SERPINA12 + LowLevel |
| 1817 | CAD ~ Intercept.High + Age + RBP4 + SERPINA12 + LowLevel |
| 1818 | CAD ~ Intercept.High + Age + RBP4 + SERPINA12 + LowLevel |
| 1819 | CAD ~ Intercept.High + Age + RBP4 + SERPINA12 + LowLevel |
| 1820 | CAD ~ Intercept.High + Age + RBP4 + SERPINA12 + LowLevel |
| 1821 | CAD ~ Intercept.High + Age + RBP4 + SERPINA12 + LowLevel |
| 1822 | CAD ~ Intercept.High + Age + RBP4 + SERPINA12 + LowLevel |
| 1823 | CAD ~ Intercept.High + Age + RBP4 + SERPINA12 + LowLevel |
| 1824 | CAD ~ Intercept.High + Age + RBP4 + SERPINA12 + LowLevel |
| 1825 | CAD ~ Intercept.High + Age + RBP4 + SERPINA12 + LowLevel |
| 1826 | CAD ~ Intercept.High + Age + RBP4 + LowLevel |
| 1827 | CAD ~ Intercept.High + Age + RBP4 + LowLevel |
| 1828 | CAD ~ Intercept.High + Age + RBP4 + LowLevel |
| 1829 | CAD ~ Intercept.High + Age + RBP4 + LowLevel |
| 1830 | CAD ~ Intercept.High + Age + RBP4 + LowLevel |
| 1831 | CAD ~ Intercept.High + Age + SERPINA12 + LowLevel |
| 1832 | CAD ~ Intercept.High + Age + SERPINA12 + LowLevel |
| 1833 | CAD ~ Intercept.High + Age + SERPINA12 + LowLevel |
| 1834 | CAD ~ Intercept.High + Age + SERPINA12 + LowLevel |
| 1835 | CAD ~ Intercept.High + Age + SERPINA12 + LowLevel |
| 1836 | CAD ~ Intercept.High + Age + LowLevel |
| 1837 | CAD ~ Intercept.High + APOB + corin + HSP70 + RBP4 + SERPINA12 + LowLevel |
| 1838 | CAD ~ Intercept.High + APOB + corin + HSP70 + RBP4 + SERPINA12 + LowLevel |
| 1839 | CAD ~ Intercept.High + APOB + corin + HSP70 + RBP4 + SERPINA12 + LowLevel |
| 1840 | CAD ~ Intercept.High + APOB + corin + HSP70 + RBP4 + SERPINA12 + LowLevel |
| 1841 | CAD ~ Intercept.High + APOB + corin + HSP70 + RBP4 + SERPINA12 + LowLevel |
| 1842 | CAD ~ Intercept.High + APOB + corin + HSP70 + RBP4 + LowLevel |
| 1843 | CAD ~ Intercept.High + APOB + corin + HSP70 + RBP4 + LowLevel |
| 1844 | CAD ~ Intercept.High + APOB + corin + HSP70 + RBP4 + LowLevel |
| 1845 | CAD ~ Intercept.High + APOB + corin + HSP70 + RBP4 + LowLevel |
| 1846 | CAD ~ Intercept.High + APOB + corin + HSP70 + RBP4 + LowLevel |
| 1847 | CAD ~ Intercept.High + APOB + corin + HSP70 + RBP4 + LowLevel |
| 1848 | CAD ~ Intercept.High + APOB + corin + HSP70 + RBP4 + LowLevel |
| 1849 | CAD ~ Intercept.High + APOB + corin + HSP70 + RBP4 + LowLevel |
| 1850 | CAD ~ Intercept.High + APOB + corin + HSP70 + RBP4 + LowLevel |
| 1851 | CAD ~ Intercept.High + APOB + corin + HSP70 + RBP4 + LowLevel |
| 1852 | CAD ~ Intercept.High + APOB + corin + HSP70 + SERPINA12 + LowLevel |
| 1853 | CAD ~ Intercept.High + APOB + corin + HSP70 + SERPINA12 + LowLevel |
| 1854 | CAD ~ Intercept.High + APOB + corin + HSP70 + SERPINA12 + LowLevel |
| 1855 | CAD ~ Intercept.High + APOB + corin + HSP70 + SERPINA12 + LowLevel |
| 1856 | CAD ~ Intercept.High + APOB + corin + HSP70 + SERPINA12 + LowLevel |
| 1857 | CAD ~ Intercept.High + APOB + corin + HSP70 + SERPINA12 + LowLevel |
| 1858 | CAD ~ Intercept.High + APOB + corin + HSP70 + SERPINA12 + LowLevel |
| 1859 | CAD ~ Intercept.High + APOB + corin + HSP70 + SERPINA12 + LowLevel |
| 1860 | CAD ~ Intercept.High + APOB + corin + HSP70 + SERPINA12 + LowLevel |
| 1861 | CAD ~ Intercept.High + APOB + corin + HSP70 + SERPINA12 + LowLevel |
| 1862 | CAD ~ Intercept.High + APOB + corin + HSP70 + LowLevel |
| 1863 | CAD ~ Intercept.High + APOB + corin + HSP70 + LowLevel |
| 1864 | CAD ~ Intercept.High + APOB + corin + HSP70 + LowLevel |
| 1865 | CAD ~ Intercept.High + APOB + corin + HSP70 + LowLevel |
| 1866 | CAD ~ Intercept.High + APOB + corin + HSP70 + LowLevel |
| 1867 | CAD ~ Intercept.High + APOB + corin + HSP70 + LowLevel |
| 1868 | CAD ~ Intercept.High + APOB + corin + HSP70 + LowLevel |
| 1869 | CAD ~ Intercept.High + APOB + corin + HSP70 + LowLevel |
| 1870 | CAD ~ Intercept.High + APOB + corin + HSP70 + LowLevel |
| 1871 | CAD ~ Intercept.High + APOB + corin + HSP70 + LowLevel |
| 1872 | CAD ~ Intercept.High + APOB + corin + RBP4 + SERPINA12 + LowLevel |
| 1873 | CAD ~ Intercept.High + APOB + corin + RBP4 + SERPINA12 + LowLevel |
| 1874 | CAD ~ Intercept.High + APOB + corin + RBP4 + SERPINA12 + LowLevel |
| 1875 | CAD ~ Intercept.High + APOB + corin + RBP4 + SERPINA12 + LowLevel |
| 1876 | CAD ~ Intercept.High + APOB + corin + RBP4 + SERPINA12 + LowLevel |
| 1877 | CAD ~ Intercept.High + APOB + corin + RBP4 + SERPINA12 + LowLevel |
| 1878 | CAD ~ Intercept.High + APOB + corin + RBP4 + SERPINA12 + LowLevel |
| 1879 | CAD ~ Intercept.High + APOB + corin + RBP4 + SERPINA12 + LowLevel |
| 1880 | CAD ~ Intercept.High + APOB + corin + RBP4 + SERPINA12 + LowLevel |
| 1881 | CAD ~ Intercept.High + APOB + corin + RBP4 + SERPINA12 + LowLevel |
| 1882 | CAD ~ Intercept.High + APOB + corin + RBP4 + LowLevel |
| 1883 | CAD ~ Intercept.High + APOB + corin + RBP4 + LowLevel |
| 1884 | CAD ~ Intercept.High + APOB + corin + RBP4 + LowLevel |
| 1885 | CAD ~ Intercept.High + APOB + corin + RBP4 + LowLevel |
| 1886 | CAD ~ Intercept.High + APOB + corin + RBP4 + LowLevel |
| 1887 | CAD ~ Intercept.High + APOB + corin + RBP4 + LowLevel |
| 1888 | CAD ~ Intercept.High + APOB + corin + RBP4 + LowLevel |
| 1889 | CAD ~ Intercept.High + APOB + corin + RBP4 + LowLevel |
| 1890 | CAD ~ Intercept.High + APOB + corin + RBP4 + LowLevel |
| 1891 | CAD ~ Intercept.High + APOB + corin + RBP4 + LowLevel |
| 1892 | CAD ~ Intercept.High + APOB + corin + SERPINA12 + LowLevel |
| 1893 | CAD ~ Intercept.High + APOB + corin + SERPINA12 + LowLevel |
| 1894 | CAD ~ Intercept.High + APOB + corin + SERPINA12 + LowLevel |
| 1895 | CAD ~ Intercept.High + APOB + corin + SERPINA12 + LowLevel |
| 1896 | CAD ~ Intercept.High + APOB + corin + SERPINA12 + LowLevel |
| 1897 | CAD ~ Intercept.High + APOB + corin + SERPINA12 + LowLevel |
| 1898 | CAD ~ Intercept.High + APOB + corin + SERPINA12 + LowLevel |
| 1899 | CAD ~ Intercept.High + APOB + corin + SERPINA12 + LowLevel |
| 1900 | CAD ~ Intercept.High + APOB + corin + SERPINA12 + LowLevel |
| 1901 | CAD ~ Intercept.High + APOB + corin + SERPINA12 + LowLevel |
| 1902 | CAD ~ Intercept.High + APOB + corin + LowLevel |
| 1903 | CAD ~ Intercept.High + APOB + corin + LowLevel |
| 1904 | CAD ~ Intercept.High + APOB + corin + LowLevel |
| 1905 | CAD ~ Intercept.High + APOB + corin + LowLevel |
| 1906 | CAD ~ Intercept.High + APOB + corin + LowLevel |
| 1907 | CAD ~ Intercept.High + APOB + HSP70 + RBP4 + SERPINA12 + LowLevel |
| 1908 | CAD ~ Intercept.High + APOB + HSP70 + RBP4 + SERPINA12 + LowLevel |
| 1909 | CAD ~ Intercept.High + APOB + HSP70 + RBP4 + SERPINA12 + LowLevel |
| 1910 | CAD ~ Intercept.High + APOB + HSP70 + RBP4 + SERPINA12 + LowLevel |
| 1911 | CAD ~ Intercept.High + APOB + HSP70 + RBP4 + SERPINA12 + LowLevel |
| 1912 | CAD ~ Intercept.High + APOB + HSP70 + RBP4 + SERPINA12 + LowLevel |
| 1913 | CAD ~ Intercept.High + APOB + HSP70 + RBP4 + SERPINA12 + LowLevel |
| 1914 | CAD ~ Intercept.High + APOB + HSP70 + RBP4 + SERPINA12 + LowLevel |
| 1915 | CAD ~ Intercept.High + APOB + HSP70 + RBP4 + SERPINA12 + LowLevel |
| 1916 | CAD ~ Intercept.High + APOB + HSP70 + RBP4 + SERPINA12 + LowLevel |
| 1917 | CAD ~ Intercept.High + APOB + HSP70 + RBP4 + LowLevel |
| 1918 | CAD ~ Intercept.High + APOB + HSP70 + RBP4 + LowLevel |
| 1919 | CAD ~ Intercept.High + APOB + HSP70 + RBP4 + LowLevel |
| 1920 | CAD ~ Intercept.High + APOB + HSP70 + RBP4 + LowLevel |
| 1921 | CAD ~ Intercept.High + APOB + HSP70 + RBP4 + LowLevel |
| 1922 | CAD ~ Intercept.High + APOB + HSP70 + RBP4 + LowLevel |
| 1923 | CAD ~ Intercept.High + APOB + HSP70 + RBP4 + LowLevel |
| 1924 | CAD ~ Intercept.High + APOB + HSP70 + RBP4 + LowLevel |
| 1925 | CAD ~ Intercept.High + APOB + HSP70 + RBP4 + LowLevel |
| 1926 | CAD ~ Intercept.High + APOB + HSP70 + RBP4 + LowLevel |
| 1927 | CAD ~ Intercept.High + APOB + HSP70 + SERPINA12 + LowLevel |
| 1928 | CAD ~ Intercept.High + APOB + HSP70 + SERPINA12 + LowLevel |
| 1929 | CAD ~ Intercept.High + APOB + HSP70 + SERPINA12 + LowLevel |
| 1930 | CAD ~ Intercept.High + APOB + HSP70 + SERPINA12 + LowLevel |
| 1931 | CAD ~ Intercept.High + APOB + HSP70 + SERPINA12 + LowLevel |
| 1932 | CAD ~ Intercept.High + APOB + HSP70 + SERPINA12 + LowLevel |
| 1933 | CAD ~ Intercept.High + APOB + HSP70 + SERPINA12 + LowLevel |
| 1934 | CAD ~ Intercept.High + APOB + HSP70 + SERPINA12 + LowLevel |
| 1935 | CAD ~ Intercept.High + APOB + HSP70 + SERPINA12 + LowLevel |
| 1936 | CAD ~ Intercept.High + APOB + HSP70 + SERPINA12 + LowLevel |
| 1937 | CAD ~ Intercept.High + APOB + HSP70 + LowLevel |
| 1938 | CAD ~ Intercept.High + APOB + HSP70 + LowLevel |
| 1939 | CAD ~ Intercept.High + APOB + HSP70 + LowLevel |
| 1940 | CAD ~ Intercept.High + APOB + HSP70 + LowLevel |
| 1941 | CAD ~ Intercept.High + APOB + HSP70 + LowLevel |
| 1942 | CAD ~ Intercept.High + APOB + RBP4 + SERPINA12 + LowLevel |
| 1943 | CAD ~ Intercept.High + APOB + RBP4 + SERPINA12 + LowLevel |
| 1944 | CAD ~ Intercept.High + APOB + RBP4 + SERPINA12 + LowLevel |
| 1945 | CAD ~ Intercept.High + APOB + RBP4 + SERPINA12 + LowLevel |
| 1946 | CAD ~ Intercept.High + APOB + RBP4 + SERPINA12 + LowLevel |
| 1947 | CAD ~ Intercept.High + APOB + RBP4 + SERPINA12 + LowLevel |
| 1948 | CAD ~ Intercept.High + APOB + RBP4 + SERPINA12 + LowLevel |
| 1949 | CAD ~ Intercept.High + APOB + RBP4 + SERPINA12 + LowLevel |
| 1950 | CAD ~ Intercept.High + APOB + RBP4 + SERPINA12 + LowLevel |
| 1951 | CAD ~ Intercept.High + APOB + RBP4 + SERPINA12 + LowLevel |
| 1952 | CAD ~ Intercept.High + APOB + RBP4 + LowLevel |
| 1953 | CAD ~ Intercept.High + APOB + RBP4 + LowLevel |
| 1954 | CAD ~ Intercept.High + APOB + RBP4 + LowLevel |
| 1955 | CAD ~ Intercept.High + APOB + RBP4 + LowLevel |
| 1956 | CAD ~ Intercept.High + APOB + RBP4 + LowLevel |
| 1957 | CAD ~ Intercept.High + APOB + SERPINA12 + LowLevel |
| 1958 | CAD ~ Intercept.High + APOB + SERPINA12 + LowLevel |
| 1959 | CAD ~ Intercept.High + APOB + SERPINA12 + LowLevel |
| 1960 | CAD ~ Intercept.High + APOB + SERPINA12 + LowLevel |
| 1961 | CAD ~ Intercept.High + APOB + SERPINA12 + LowLevel |
| 1962 | CAD ~ Intercept.High + APOB + LowLevel |
| 1963 | CAD ~ Intercept.High + corin + HSP70 + RBP4 + SERPINA12 + LowLevel |
| 1964 | CAD ~ Intercept.High + corin + HSP70 + RBP4 + SERPINA12 + LowLevel |
| 1965 | CAD ~ Intercept.High + corin + HSP70 + RBP4 + SERPINA12 + LowLevel |
| 1966 | CAD ~ Intercept.High + corin + HSP70 + RBP4 + SERPINA12 + LowLevel |
| 1967 | CAD ~ Intercept.High + corin + HSP70 + RBP4 + SERPINA12 + LowLevel |
| 1968 | CAD ~ Intercept.High + corin + HSP70 + RBP4 + SERPINA12 + LowLevel |
| 1969 | CAD ~ Intercept.High + corin + HSP70 + RBP4 + SERPINA12 + LowLevel |
| 1970 | CAD ~ Intercept.High + corin + HSP70 + RBP4 + SERPINA12 + LowLevel |
| 1971 | CAD ~ Intercept.High + corin + HSP70 + RBP4 + SERPINA12 + LowLevel |
| 1972 | CAD ~ Intercept.High + corin + HSP70 + RBP4 + SERPINA12 + LowLevel |
| 1973 | CAD ~ Intercept.High + corin + HSP70 + RBP4 + LowLevel |
| 1974 | CAD ~ Intercept.High + corin + HSP70 + RBP4 + LowLevel |
| 1975 | CAD ~ Intercept.High + corin + HSP70 + RBP4 + LowLevel |
| 1976 | CAD ~ Intercept.High + corin + HSP70 + RBP4 + LowLevel |
| 1977 | CAD ~ Intercept.High + corin + HSP70 + RBP4 + LowLevel |
| 1978 | CAD ~ Intercept.High + corin + HSP70 + RBP4 + LowLevel |
| 1979 | CAD ~ Intercept.High + corin + HSP70 + RBP4 + LowLevel |
| 1980 | CAD ~ Intercept.High + corin + HSP70 + RBP4 + LowLevel |
| 1981 | CAD ~ Intercept.High + corin + HSP70 + RBP4 + LowLevel |
| 1982 | CAD ~ Intercept.High + corin + HSP70 + RBP4 + LowLevel |
| 1983 | CAD ~ Intercept.High + corin + HSP70 + SERPINA12 + LowLevel |
| 1984 | CAD ~ Intercept.High + corin + HSP70 + SERPINA12 + LowLevel |
| 1985 | CAD ~ Intercept.High + corin + HSP70 + SERPINA12 + LowLevel |
| 1986 | CAD ~ Intercept.High + corin + HSP70 + SERPINA12 + LowLevel |
| 1987 | CAD ~ Intercept.High + corin + HSP70 + SERPINA12 + LowLevel |
| 1988 | CAD ~ Intercept.High + corin + HSP70 + SERPINA12 + LowLevel |
| 1989 | CAD ~ Intercept.High + corin + HSP70 + SERPINA12 + LowLevel |
| 1990 | CAD ~ Intercept.High + corin + HSP70 + SERPINA12 + LowLevel |
| 1991 | CAD ~ Intercept.High + corin + HSP70 + SERPINA12 + LowLevel |
| 1992 | CAD ~ Intercept.High + corin + HSP70 + SERPINA12 + LowLevel |
| 1993 | CAD ~ Intercept.High + corin + HSP70 + LowLevel |
| 1994 | CAD ~ Intercept.High + corin + HSP70 + LowLevel |
| 1995 | CAD ~ Intercept.High + corin + HSP70 + LowLevel |
| 1996 | CAD ~ Intercept.High + corin + HSP70 + LowLevel |
| 1997 | CAD ~ Intercept.High + corin + HSP70 + LowLevel |
| 1998 | CAD ~ Intercept.High + corin + RBP4 + SERPINA12 + LowLevel |
| 1999 | CAD ~ Intercept.High + corin + RBP4 + SERPINA12 + LowLevel |
| 2000 | CAD ~ Intercept.High + corin + RBP4 + SERPINA12 + LowLevel |
| 2001 | CAD ~ Intercept.High + corin + RBP4 + SERPINA12 + LowLevel |
| 2002 | CAD ~ Intercept.High + corin + RBP4 + SERPINA12 + LowLevel |
| 2003 | CAD ~ Intercept.High + corin + RBP4 + SERPINA12 + LowLevel |
| 2004 | CAD ~ Intercept.High + corin + RBP4 + SERPINA12 + LowLevel |
| 2005 | CAD ~ Intercept.High + corin + RBP4 + SERPINA12 + LowLevel |
| 2006 | CAD ~ Intercept.High + corin + RBP4 + SERPINA12 + LowLevel |
| 2007 | CAD ~ Intercept.High + corin + RBP4 + SERPINA12 + LowLevel |
| 2008 | CAD ~ Intercept.High + corin + RBP4 + LowLevel |
| 2009 | CAD ~ Intercept.High + corin + RBP4 + LowLevel |
| 2010 | CAD ~ Intercept.High + corin + RBP4 + LowLevel |
| 2011 | CAD ~ Intercept.High + corin + RBP4 + LowLevel |
| 2012 | CAD ~ Intercept.High + corin + RBP4 + LowLevel |
| 2013 | CAD ~ Intercept.High + corin + SERPINA12 + LowLevel |
| 2014 | CAD ~ Intercept.High + corin + SERPINA12 + LowLevel |
| 2015 | CAD ~ Intercept.High + corin + SERPINA12 + LowLevel |
| 2016 | CAD ~ Intercept.High + corin + SERPINA12 + LowLevel |
| 2017 | CAD ~ Intercept.High + corin + SERPINA12 + LowLevel |
| 2018 | CAD ~ Intercept.High + corin + LowLevel |
| 2019 | CAD ~ Intercept.High + Sex + Age + APOB + corin + HSP70 + LowLevel |
| 2020 | CAD ~ Intercept.High + Sex + Age + APOB + corin + HSP70 + LowLevel |
| 2021 | CAD ~ Intercept.High + Sex + Age + APOB + corin + HSP70 + LowLevel |
| 2022 | CAD ~ Intercept.High + Sex + Age + APOB + corin + HSP70 + LowLevel |
| 2023 | CAD ~ Intercept.High + Sex + Age + APOB + corin + HSP70 + LowLevel |
| 2024 | CAD ~ Intercept.High + Sex + Age + APOB + corin + RBP4 + LowLevel |
| 2025 | CAD ~ Intercept.High + Sex + Age + APOB + corin + RBP4 + LowLevel |
| 2026 | CAD ~ Intercept.High + Sex + Age + APOB + corin + RBP4 + LowLevel |
| 2027 | CAD ~ Intercept.High + Sex + Age + APOB + corin + RBP4 + LowLevel |
| 2028 | CAD ~ Intercept.High + Sex + Age + APOB + corin + RBP4 + LowLevel |
| 2029 | CAD ~ Intercept.High + Sex + Age + APOB + corin + SERPINA12 + LowLevel |
| 2030 | CAD ~ Intercept.High + Sex + Age + APOB + corin + SERPINA12 + LowLevel |
| 2031 | CAD ~ Intercept.High + Sex + Age + APOB + corin + SERPINA12 + LowLevel |
| 2032 | CAD ~ Intercept.High + Sex + Age + APOB + corin + SERPINA12 + LowLevel |
| 2033 | CAD ~ Intercept.High + Sex + Age + APOB + corin + SERPINA12 + LowLevel |
| 2034 | CAD ~ Intercept.High + Sex + Age + APOB + corin + LowLevel |
| 2035 | CAD ~ Intercept.High + Sex + Age + APOB + corin + LowLevel |
| 2036 | CAD ~ Intercept.High + Sex + Age + APOB + corin + LowLevel |
| 2037 | CAD ~ Intercept.High + Sex + Age + APOB + corin + LowLevel |
| 2038 | CAD ~ Intercept.High + Sex + Age + APOB + corin + LowLevel |
| 2039 | CAD ~ Intercept.High + Sex + Age + APOB + corin + LowLevel |
| 2040 | CAD ~ Intercept.High + Sex + Age + APOB + corin + LowLevel |
| 2041 | CAD ~ Intercept.High + Sex + Age + APOB + corin + LowLevel |
| 2042 | CAD ~ Intercept.High + Sex + Age + APOB + corin + LowLevel |
| 2043 | CAD ~ Intercept.High + Sex + Age + APOB + corin + LowLevel |
| 2044 | CAD ~ Intercept.High + Sex + Age + APOB + HSP70 + RBP4 + LowLevel |
| 2045 | CAD ~ Intercept.High + Sex + Age + APOB + HSP70 + RBP4 + LowLevel |
| 2046 | CAD ~ Intercept.High + Sex + Age + APOB + HSP70 + RBP4 + LowLevel |
| 2047 | CAD ~ Intercept.High + Sex + Age + APOB + HSP70 + RBP4 + LowLevel |
| 2048 | CAD ~ Intercept.High + Sex + Age + APOB + HSP70 + RBP4 + LowLevel |
| 2049 | CAD ~ Intercept.High + Sex + Age + APOB + HSP70 + SERPINA12 + LowLevel |
| 2050 | CAD ~ Intercept.High + Sex + Age + APOB + HSP70 + SERPINA12 + LowLevel |
| 2051 | CAD ~ Intercept.High + Sex + Age + APOB + HSP70 + SERPINA12 + LowLevel |
| 2052 | CAD ~ Intercept.High + Sex + Age + APOB + HSP70 + SERPINA12 + LowLevel |
| 2053 | CAD ~ Intercept.High + Sex + Age + APOB + HSP70 + SERPINA12 + LowLevel |
| 2054 | CAD ~ Intercept.High + Sex + Age + APOB + HSP70 + LowLevel |
| 2055 | CAD ~ Intercept.High + Sex + Age + APOB + HSP70 + LowLevel |
| 2056 | CAD ~ Intercept.High + Sex + Age + APOB + HSP70 + LowLevel |
| 2057 | CAD ~ Intercept.High + Sex + Age + APOB + HSP70 + LowLevel |
| 2058 | CAD ~ Intercept.High + Sex + Age + APOB + HSP70 + LowLevel |
| 2059 | CAD ~ Intercept.High + Sex + Age + APOB + HSP70 + LowLevel |
| 2060 | CAD ~ Intercept.High + Sex + Age + APOB + HSP70 + LowLevel |
| 2061 | CAD ~ Intercept.High + Sex + Age + APOB + HSP70 + LowLevel |
| 2062 | CAD ~ Intercept.High + Sex + Age + APOB + HSP70 + LowLevel |
| 2063 | CAD ~ Intercept.High + Sex + Age + APOB + HSP70 + LowLevel |
| 2064 | CAD ~ Intercept.High + Sex + Age + APOB + RBP4 + SERPINA12 + LowLevel |
| 2065 | CAD ~ Intercept.High + Sex + Age + APOB + RBP4 + SERPINA12 + LowLevel |
| 2066 | CAD ~ Intercept.High + Sex + Age + APOB + RBP4 + SERPINA12 + LowLevel |
| 2067 | CAD ~ Intercept.High + Sex + Age + APOB + RBP4 + SERPINA12 + LowLevel |
| 2068 | CAD ~ Intercept.High + Sex + Age + APOB + RBP4 + SERPINA12 + LowLevel |
| 2069 | CAD ~ Intercept.High + Sex + Age + APOB + RBP4 + LowLevel |
| 2070 | CAD ~ Intercept.High + Sex + Age + APOB + RBP4 + LowLevel |
| 2071 | CAD ~ Intercept.High + Sex + Age + APOB + RBP4 + LowLevel |
| 2072 | CAD ~ Intercept.High + Sex + Age + APOB + RBP4 + LowLevel |
| 2073 | CAD ~ Intercept.High + Sex + Age + APOB + RBP4 + LowLevel |
| 2074 | CAD ~ Intercept.High + Sex + Age + APOB + RBP4 + LowLevel |
| 2075 | CAD ~ Intercept.High + Sex + Age + APOB + RBP4 + LowLevel |
| 2076 | CAD ~ Intercept.High + Sex + Age + APOB + RBP4 + LowLevel |
| 2077 | CAD ~ Intercept.High + Sex + Age + APOB + RBP4 + LowLevel |
| 2078 | CAD ~ Intercept.High + Sex + Age + APOB + RBP4 + LowLevel |
| 2079 | CAD ~ Intercept.High + Sex + Age + APOB + SERPINA12 + LowLevel |
| 2080 | CAD ~ Intercept.High + Sex + Age + APOB + SERPINA12 + LowLevel |
| 2081 | CAD ~ Intercept.High + Sex + Age + APOB + SERPINA12 + LowLevel |
| 2082 | CAD ~ Intercept.High + Sex + Age + APOB + SERPINA12 + LowLevel |
| 2083 | CAD ~ Intercept.High + Sex + Age + APOB + SERPINA12 + LowLevel |
| 2084 | CAD ~ Intercept.High + Sex + Age + APOB + SERPINA12 + LowLevel |
| 2085 | CAD ~ Intercept.High + Sex + Age + APOB + SERPINA12 + LowLevel |
| 2086 | CAD ~ Intercept.High + Sex + Age + APOB + SERPINA12 + LowLevel |
| 2087 | CAD ~ Intercept.High + Sex + Age + APOB + SERPINA12 + LowLevel |
| 2088 | CAD ~ Intercept.High + Sex + Age + APOB + SERPINA12 + LowLevel |
| 2089 | CAD ~ Intercept.High + Sex + Age + APOB + LowLevel |
| 2090 | CAD ~ Intercept.High + Sex + Age + APOB + LowLevel |
| 2091 | CAD ~ Intercept.High + Sex + Age + APOB + LowLevel |
| 2092 | CAD ~ Intercept.High + Sex + Age + APOB + LowLevel |
| 2093 | CAD ~ Intercept.High + Sex + Age + APOB + LowLevel |
| 2094 | CAD ~ Intercept.High + Sex + Age + APOB + LowLevel |
| 2095 | CAD ~ Intercept.High + Sex + Age + APOB + LowLevel |
| 2096 | CAD ~ Intercept.High + Sex + Age + APOB + LowLevel |
| 2097 | CAD ~ Intercept.High + Sex + Age + APOB + LowLevel |
| 2098 | CAD ~ Intercept.High + Sex + Age + APOB + LowLevel |
| 2099 | CAD ~ Intercept.High + Sex + Age + corin + HSP70 + RBP4 + LowLevel |
| 2100 | CAD ~ Intercept.High + Sex + Age + corin + HSP70 + RBP4 + LowLevel |
| 2101 | CAD ~ Intercept.High + Sex + Age + corin + HSP70 + RBP4 + LowLevel |
| 2102 | CAD ~ Intercept.High + Sex + Age + corin + HSP70 + RBP4 + LowLevel |
| 2103 | CAD ~ Intercept.High + Sex + Age + corin + HSP70 + RBP4 + LowLevel |
| 2104 | CAD ~ Intercept.High + Sex + Age + corin + HSP70 + SERPINA12 + LowLevel |
| 2105 | CAD ~ Intercept.High + Sex + Age + corin + HSP70 + SERPINA12 + LowLevel |
| 2106 | CAD ~ Intercept.High + Sex + Age + corin + HSP70 + SERPINA12 + LowLevel |
| 2107 | CAD ~ Intercept.High + Sex + Age + corin + HSP70 + SERPINA12 + LowLevel |
| 2108 | CAD ~ Intercept.High + Sex + Age + corin + HSP70 + SERPINA12 + LowLevel |
| 2109 | CAD ~ Intercept.High + Sex + Age + corin + HSP70 + LowLevel |
| 2110 | CAD ~ Intercept.High + Sex + Age + corin + HSP70 + LowLevel |
| 2111 | CAD ~ Intercept.High + Sex + Age + corin + HSP70 + LowLevel |
| 2112 | CAD ~ Intercept.High + Sex + Age + corin + HSP70 + LowLevel |
| 2113 | CAD ~ Intercept.High + Sex + Age + corin + HSP70 + LowLevel |
| 2114 | CAD ~ Intercept.High + Sex + Age + corin + HSP70 + LowLevel |
| 2115 | CAD ~ Intercept.High + Sex + Age + corin + HSP70 + LowLevel |
| 2116 | CAD ~ Intercept.High + Sex + Age + corin + HSP70 + LowLevel |
| 2117 | CAD ~ Intercept.High + Sex + Age + corin + HSP70 + LowLevel |
| 2118 | CAD ~ Intercept.High + Sex + Age + corin + HSP70 + LowLevel |
| 2119 | CAD ~ Intercept.High + Sex + Age + corin + RBP4 + SERPINA12 + LowLevel |
| 2120 | CAD ~ Intercept.High + Sex + Age + corin + RBP4 + SERPINA12 + LowLevel |
| 2121 | CAD ~ Intercept.High + Sex + Age + corin + RBP4 + SERPINA12 + LowLevel |
| 2122 | CAD ~ Intercept.High + Sex + Age + corin + RBP4 + SERPINA12 + LowLevel |
| 2123 | CAD ~ Intercept.High + Sex + Age + corin + RBP4 + SERPINA12 + LowLevel |
| 2124 | CAD ~ Intercept.High + Sex + Age + corin + RBP4 + LowLevel |
| 2125 | CAD ~ Intercept.High + Sex + Age + corin + RBP4 + LowLevel |
| 2126 | CAD ~ Intercept.High + Sex + Age + corin + RBP4 + LowLevel |
| 2127 | CAD ~ Intercept.High + Sex + Age + corin + RBP4 + LowLevel |
| 2128 | CAD ~ Intercept.High + Sex + Age + corin + RBP4 + LowLevel |
| 2129 | CAD ~ Intercept.High + Sex + Age + corin + RBP4 + LowLevel |
| 2130 | CAD ~ Intercept.High + Sex + Age + corin + RBP4 + LowLevel |
| 2131 | CAD ~ Intercept.High + Sex + Age + corin + RBP4 + LowLevel |
| 2132 | CAD ~ Intercept.High + Sex + Age + corin + RBP4 + LowLevel |
| 2133 | CAD ~ Intercept.High + Sex + Age + corin + RBP4 + LowLevel |
| 2134 | CAD ~ Intercept.High + Sex + Age + corin + SERPINA12 + LowLevel |
| 2135 | CAD ~ Intercept.High + Sex + Age + corin + SERPINA12 + LowLevel |
| 2136 | CAD ~ Intercept.High + Sex + Age + corin + SERPINA12 + LowLevel |
| 2137 | CAD ~ Intercept.High + Sex + Age + corin + SERPINA12 + LowLevel |
| 2138 | CAD ~ Intercept.High + Sex + Age + corin + SERPINA12 + LowLevel |
| 2139 | CAD ~ Intercept.High + Sex + Age + corin + SERPINA12 + LowLevel |
| 2140 | CAD ~ Intercept.High + Sex + Age + corin + SERPINA12 + LowLevel |
| 2141 | CAD ~ Intercept.High + Sex + Age + corin + SERPINA12 + LowLevel |
| 2142 | CAD ~ Intercept.High + Sex + Age + corin + SERPINA12 + LowLevel |
| 2143 | CAD ~ Intercept.High + Sex + Age + corin + SERPINA12 + LowLevel |
| 2144 | CAD ~ Intercept.High + Sex + Age + corin + LowLevel |
| 2145 | CAD ~ Intercept.High + Sex + Age + corin + LowLevel |
| 2146 | CAD ~ Intercept.High + Sex + Age + corin + LowLevel |
| 2147 | CAD ~ Intercept.High + Sex + Age + corin + LowLevel |
| 2148 | CAD ~ Intercept.High + Sex + Age + corin + LowLevel |
| 2149 | CAD ~ Intercept.High + Sex + Age + corin + LowLevel |
| 2150 | CAD ~ Intercept.High + Sex + Age + corin + LowLevel |
| 2151 | CAD ~ Intercept.High + Sex + Age + corin + LowLevel |
| 2152 | CAD ~ Intercept.High + Sex + Age + corin + LowLevel |
| 2153 | CAD ~ Intercept.High + Sex + Age + corin + LowLevel |
| 2154 | CAD ~ Intercept.High + Sex + Age + HSP70 + RBP4 + SERPINA12 + LowLevel |
| 2155 | CAD ~ Intercept.High + Sex + Age + HSP70 + RBP4 + SERPINA12 + LowLevel |
| 2156 | CAD ~ Intercept.High + Sex + Age + HSP70 + RBP4 + SERPINA12 + LowLevel |
| 2157 | CAD ~ Intercept.High + Sex + Age + HSP70 + RBP4 + SERPINA12 + LowLevel |
| 2158 | CAD ~ Intercept.High + Sex + Age + HSP70 + RBP4 + SERPINA12 + LowLevel |
| 2159 | CAD ~ Intercept.High + Sex + Age + HSP70 + RBP4 + LowLevel |
| 2160 | CAD ~ Intercept.High + Sex + Age + HSP70 + RBP4 + LowLevel |
| 2161 | CAD ~ Intercept.High + Sex + Age + HSP70 + RBP4 + LowLevel |
| 2162 | CAD ~ Intercept.High + Sex + Age + HSP70 + RBP4 + LowLevel |
| 2163 | CAD ~ Intercept.High + Sex + Age + HSP70 + RBP4 + LowLevel |
| 2164 | CAD ~ Intercept.High + Sex + Age + HSP70 + RBP4 + LowLevel |
| 2165 | CAD ~ Intercept.High + Sex + Age + HSP70 + RBP4 + LowLevel |
| 2166 | CAD ~ Intercept.High + Sex + Age + HSP70 + RBP4 + LowLevel |
| 2167 | CAD ~ Intercept.High + Sex + Age + HSP70 + RBP4 + LowLevel |
| 2168 | CAD ~ Intercept.High + Sex + Age + HSP70 + RBP4 + LowLevel |
| 2169 | CAD ~ Intercept.High + Sex + Age + HSP70 + SERPINA12 + LowLevel |
| 2170 | CAD ~ Intercept.High + Sex + Age + HSP70 + SERPINA12 + LowLevel |
| 2171 | CAD ~ Intercept.High + Sex + Age + HSP70 + SERPINA12 + LowLevel |
| 2172 | CAD ~ Intercept.High + Sex + Age + HSP70 + SERPINA12 + LowLevel |
| 2173 | CAD ~ Intercept.High + Sex + Age + HSP70 + SERPINA12 + LowLevel |
| 2174 | CAD ~ Intercept.High + Sex + Age + HSP70 + SERPINA12 + LowLevel |
| 2175 | CAD ~ Intercept.High + Sex + Age + HSP70 + SERPINA12 + LowLevel |
| 2176 | CAD ~ Intercept.High + Sex + Age + HSP70 + SERPINA12 + LowLevel |
| 2177 | CAD ~ Intercept.High + Sex + Age + HSP70 + SERPINA12 + LowLevel |
| 2178 | CAD ~ Intercept.High + Sex + Age + HSP70 + SERPINA12 + LowLevel |
| 2179 | CAD ~ Intercept.High + Sex + Age + HSP70 + LowLevel |
| 2180 | CAD ~ Intercept.High + Sex + Age + HSP70 + LowLevel |
| 2181 | CAD ~ Intercept.High + Sex + Age + HSP70 + LowLevel |
| 2182 | CAD ~ Intercept.High + Sex + Age + HSP70 + LowLevel |
| 2183 | CAD ~ Intercept.High + Sex + Age + HSP70 + LowLevel |
| 2184 | CAD ~ Intercept.High + Sex + Age + HSP70 + LowLevel |
| 2185 | CAD ~ Intercept.High + Sex + Age + HSP70 + LowLevel |
| 2186 | CAD ~ Intercept.High + Sex + Age + HSP70 + LowLevel |
| 2187 | CAD ~ Intercept.High + Sex + Age + HSP70 + LowLevel |
| 2188 | CAD ~ Intercept.High + Sex + Age + HSP70 + LowLevel |
| 2189 | CAD ~ Intercept.High + Sex + Age + RBP4 + SERPINA12 + LowLevel |
| 2190 | CAD ~ Intercept.High + Sex + Age + RBP4 + SERPINA12 + LowLevel |
| 2191 | CAD ~ Intercept.High + Sex + Age + RBP4 + SERPINA12 + LowLevel |
| 2192 | CAD ~ Intercept.High + Sex + Age + RBP4 + SERPINA12 + LowLevel |
| 2193 | CAD ~ Intercept.High + Sex + Age + RBP4 + SERPINA12 + LowLevel |
| 2194 | CAD ~ Intercept.High + Sex + Age + RBP4 + SERPINA12 + LowLevel |
| 2195 | CAD ~ Intercept.High + Sex + Age + RBP4 + SERPINA12 + LowLevel |
| 2196 | CAD ~ Intercept.High + Sex + Age + RBP4 + SERPINA12 + LowLevel |
| 2197 | CAD ~ Intercept.High + Sex + Age + RBP4 + SERPINA12 + LowLevel |
| 2198 | CAD ~ Intercept.High + Sex + Age + RBP4 + SERPINA12 + LowLevel |
| 2199 | CAD ~ Intercept.High + Sex + Age + RBP4 + LowLevel |
| 2200 | CAD ~ Intercept.High + Sex + Age + RBP4 + LowLevel |
| 2201 | CAD ~ Intercept.High + Sex + Age + RBP4 + LowLevel |
| 2202 | CAD ~ Intercept.High + Sex + Age + RBP4 + LowLevel |
| 2203 | CAD ~ Intercept.High + Sex + Age + RBP4 + LowLevel |
| 2204 | CAD ~ Intercept.High + Sex + Age + RBP4 + LowLevel |
| 2205 | CAD ~ Intercept.High + Sex + Age + RBP4 + LowLevel |
| 2206 | CAD ~ Intercept.High + Sex + Age + RBP4 + LowLevel |
| 2207 | CAD ~ Intercept.High + Sex + Age + RBP4 + LowLevel |
| 2208 | CAD ~ Intercept.High + Sex + Age + RBP4 + LowLevel |
| 2209 | CAD ~ Intercept.High + Sex + Age + SERPINA12 + LowLevel |
| 2210 | CAD ~ Intercept.High + Sex + Age + SERPINA12 + LowLevel |
| 2211 | CAD ~ Intercept.High + Sex + Age + SERPINA12 + LowLevel |
| 2212 | CAD ~ Intercept.High + Sex + Age + SERPINA12 + LowLevel |
| 2213 | CAD ~ Intercept.High + Sex + Age + SERPINA12 + LowLevel |
| 2214 | CAD ~ Intercept.High + Sex + Age + SERPINA12 + LowLevel |
| 2215 | CAD ~ Intercept.High + Sex + Age + SERPINA12 + LowLevel |
| 2216 | CAD ~ Intercept.High + Sex + Age + SERPINA12 + LowLevel |
| 2217 | CAD ~ Intercept.High + Sex + Age + SERPINA12 + LowLevel |
| 2218 | CAD ~ Intercept.High + Sex + Age + SERPINA12 + LowLevel |
| 2219 | CAD ~ Intercept.High + Sex + Age + LowLevel |
| 2220 | CAD ~ Intercept.High + Sex + Age + LowLevel |
| 2221 | CAD ~ Intercept.High + Sex + Age + LowLevel |
| 2222 | CAD ~ Intercept.High + Sex + Age + LowLevel |
| 2223 | CAD ~ Intercept.High + Sex + Age + LowLevel |
| 2224 | CAD ~ Intercept.High + Sex + APOB + corin + HSP70 + RBP4 + LowLevel |
| 2225 | CAD ~ Intercept.High + Sex + APOB + corin + HSP70 + RBP4 + LowLevel |
| 2226 | CAD ~ Intercept.High + Sex + APOB + corin + HSP70 + RBP4 + LowLevel |
| 2227 | CAD ~ Intercept.High + Sex + APOB + corin + HSP70 + RBP4 + LowLevel |
| 2228 | CAD ~ Intercept.High + Sex + APOB + corin + HSP70 + RBP4 + LowLevel |
| 2229 | CAD ~ Intercept.High + Sex + APOB + corin + HSP70 + SERPINA12 + LowLevel |
| 2230 | CAD ~ Intercept.High + Sex + APOB + corin + HSP70 + SERPINA12 + LowLevel |
| 2231 | CAD ~ Intercept.High + Sex + APOB + corin + HSP70 + SERPINA12 + LowLevel |
| 2232 | CAD ~ Intercept.High + Sex + APOB + corin + HSP70 + SERPINA12 + LowLevel |
| 2233 | CAD ~ Intercept.High + Sex + APOB + corin + HSP70 + SERPINA12 + LowLevel |
| 2234 | CAD ~ Intercept.High + Sex + APOB + corin + HSP70 + LowLevel |
| 2235 | CAD ~ Intercept.High + Sex + APOB + corin + HSP70 + LowLevel |
| 2236 | CAD ~ Intercept.High + Sex + APOB + corin + HSP70 + LowLevel |
| 2237 | CAD ~ Intercept.High + Sex + APOB + corin + HSP70 + LowLevel |
| 2238 | CAD ~ Intercept.High + Sex + APOB + corin + HSP70 + LowLevel |
| 2239 | CAD ~ Intercept.High + Sex + APOB + corin + HSP70 + LowLevel |
| 2240 | CAD ~ Intercept.High + Sex + APOB + corin + HSP70 + LowLevel |
| 2241 | CAD ~ Intercept.High + Sex + APOB + corin + HSP70 + LowLevel |
| 2242 | CAD ~ Intercept.High + Sex + APOB + corin + HSP70 + LowLevel |
| 2243 | CAD ~ Intercept.High + Sex + APOB + corin + HSP70 + LowLevel |
| 2244 | CAD ~ Intercept.High + Sex + APOB + corin + RBP4 + SERPINA12 + LowLevel |
| 2245 | CAD ~ Intercept.High + Sex + APOB + corin + RBP4 + SERPINA12 + LowLevel |
| 2246 | CAD ~ Intercept.High + Sex + APOB + corin + RBP4 + SERPINA12 + LowLevel |
| 2247 | CAD ~ Intercept.High + Sex + APOB + corin + RBP4 + SERPINA12 + LowLevel |
| 2248 | CAD ~ Intercept.High + Sex + APOB + corin + RBP4 + SERPINA12 + LowLevel |
| 2249 | CAD ~ Intercept.High + Sex + APOB + corin + RBP4 + LowLevel |
| 2250 | CAD ~ Intercept.High + Sex + APOB + corin + RBP4 + LowLevel |
| 2251 | CAD ~ Intercept.High + Sex + APOB + corin + RBP4 + LowLevel |
| 2252 | CAD ~ Intercept.High + Sex + APOB + corin + RBP4 + LowLevel |
| 2253 | CAD ~ Intercept.High + Sex + APOB + corin + RBP4 + LowLevel |
| 2254 | CAD ~ Intercept.High + Sex + APOB + corin + RBP4 + LowLevel |
| 2255 | CAD ~ Intercept.High + Sex + APOB + corin + RBP4 + LowLevel |
| 2256 | CAD ~ Intercept.High + Sex + APOB + corin + RBP4 + LowLevel |
| 2257 | CAD ~ Intercept.High + Sex + APOB + corin + RBP4 + LowLevel |
| 2258 | CAD ~ Intercept.High + Sex + APOB + corin + RBP4 + LowLevel |
| 2259 | CAD ~ Intercept.High + Sex + APOB + corin + SERPINA12 + LowLevel |
| 2260 | CAD ~ Intercept.High + Sex + APOB + corin + SERPINA12 + LowLevel |
| 2261 | CAD ~ Intercept.High + Sex + APOB + corin + SERPINA12 + LowLevel |
| 2262 | CAD ~ Intercept.High + Sex + APOB + corin + SERPINA12 + LowLevel |
| 2263 | CAD ~ Intercept.High + Sex + APOB + corin + SERPINA12 + LowLevel |
| 2264 | CAD ~ Intercept.High + Sex + APOB + corin + SERPINA12 + LowLevel |
| 2265 | CAD ~ Intercept.High + Sex + APOB + corin + SERPINA12 + LowLevel |
| 2266 | CAD ~ Intercept.High + Sex + APOB + corin + SERPINA12 + LowLevel |
| 2267 | CAD ~ Intercept.High + Sex + APOB + corin + SERPINA12 + LowLevel |
| 2268 | CAD ~ Intercept.High + Sex + APOB + corin + SERPINA12 + LowLevel |
| 2269 | CAD ~ Intercept.High + Sex + APOB + corin + LowLevel |
| 2270 | CAD ~ Intercept.High + Sex + APOB + corin + LowLevel |
| 2271 | CAD ~ Intercept.High + Sex + APOB + corin + LowLevel |
| 2272 | CAD ~ Intercept.High + Sex + APOB + corin + LowLevel |
| 2273 | CAD ~ Intercept.High + Sex + APOB + corin + LowLevel |
| 2274 | CAD ~ Intercept.High + Sex + APOB + corin + LowLevel |
| 2275 | CAD ~ Intercept.High + Sex + APOB + corin + LowLevel |
| 2276 | CAD ~ Intercept.High + Sex + APOB + corin + LowLevel |
| 2277 | CAD ~ Intercept.High + Sex + APOB + corin + LowLevel |
| 2278 | CAD ~ Intercept.High + Sex + APOB + corin + LowLevel |
| 2279 | CAD ~ Intercept.High + Sex + APOB + HSP70 + RBP4 + SERPINA12 + LowLevel |
| 2280 | CAD ~ Intercept.High + Sex + APOB + HSP70 + RBP4 + SERPINA12 + LowLevel |
| 2281 | CAD ~ Intercept.High + Sex + APOB + HSP70 + RBP4 + SERPINA12 + LowLevel |
| 2282 | CAD ~ Intercept.High + Sex + APOB + HSP70 + RBP4 + SERPINA12 + LowLevel |
| 2283 | CAD ~ Intercept.High + Sex + APOB + HSP70 + RBP4 + SERPINA12 + LowLevel |
| 2284 | CAD ~ Intercept.High + Sex + APOB + HSP70 + RBP4 + LowLevel |
| 2285 | CAD ~ Intercept.High + Sex + APOB + HSP70 + RBP4 + LowLevel |
| 2286 | CAD ~ Intercept.High + Sex + APOB + HSP70 + RBP4 + LowLevel |
| 2287 | CAD ~ Intercept.High + Sex + APOB + HSP70 + RBP4 + LowLevel |
| 2288 | CAD ~ Intercept.High + Sex + APOB + HSP70 + RBP4 + LowLevel |
| 2289 | CAD ~ Intercept.High + Sex + APOB + HSP70 + RBP4 + LowLevel |
| 2290 | CAD ~ Intercept.High + Sex + APOB + HSP70 + RBP4 + LowLevel |
| 2291 | CAD ~ Intercept.High + Sex + APOB + HSP70 + RBP4 + LowLevel |
| 2292 | CAD ~ Intercept.High + Sex + APOB + HSP70 + RBP4 + LowLevel |
| 2293 | CAD ~ Intercept.High + Sex + APOB + HSP70 + RBP4 + LowLevel |
| 2294 | CAD ~ Intercept.High + Sex + APOB + HSP70 + SERPINA12 + LowLevel |
| 2295 | CAD ~ Intercept.High + Sex + APOB + HSP70 + SERPINA12 + LowLevel |
| 2296 | CAD ~ Intercept.Hiah + Sex + APOB + HSP70 + SERPINA12 + LowLevel |
| 2297 | CAD ~ Intercept.High + Sex + APOB + HSP70 + SERPINA12 + LowLevel |
| 2298 | CAD ~ Intercept.High + Sex + APOB + HSP70 + SERPINA12 + LowLevel |
| 2299 | CAD ~ Intercept.High + Sex + APOB + HSP70 + SERPINA12 + LowLevel |
| 2300 | CAD ~ Intercept.High + Sex + APOB + HSP70 + SERPINA12 + LowLevel |
| 2301 | CAD ~ Intercept.High + Sex + APOB + HSP70 + SERPINA12 + LowLevel |
| 2302 | CAD ~ Intercept.High + Sex + APOB + HSP70 + SERPINA12 + LowLevel |
| 2303 | CAD ~ Intercept.High + Sex + APOB + HSP70 + SERPINA12 + LowLevel |
| 2304 | CAD ~ Intercept.High + Sex + APOB + HSP70 + LowLevel |
| 2305 | CAD ~ Intercept.High + Sex + APOB + HSP70 + LowLevel |
| 2306 | CAD ~ Intercept.High + Sex + APOB + HSP70 + LowLevel |
| 2307 | CAD ~ Intercept.High + Sex + APOB + HSP70 + LowLevel |
| 2308 | CAD ~ Intercept.High + Sex + APOB + HSP70 + LowLevel |
| 2309 | CAD ~ Intercept.High + Sex + APOB + HSP70 + LowLevel |
| 2310 | CAD ~ Intercept.High + Sex + APOB + HSP70 + LowLevel |
| 2311 | CAD ~ Intercept.High + Sex + APOB + HSP70 + LowLevel |
| 2312 | CAD ~ Intercept.High + Sex + APOB + HSP70 + LowLevel |
| 2313 | CAD ~ Intercept.High + Sex + APOB + HSP70 + LowLevel |
| 2314 | CAD ~ Intercept.High + Sex + APOB + RBP4 + SERPINA12 + LowLevel |
| 2315 | CAD ~ Intercept.High + Sex + APOB + RBP4 + SERPINA12 + LowLevel |
| 2316 | CAD ~ Intercept.High + Sex + APOB + RBP4 + SERPINA12 + LowLevel |
| 2317 | CAD ~ Intercept.High + Sex + APOB + RBP4 + SERPINA12 + LowLevel |
| 2318 | CAD ~ Intercept.High + Sex + APOB + RBP4 + SERPINA12 + LowLevel |
| 2319 | CAD ~ Intercept.High + Sex + APOB + RBP4 + SERPINA12 + LowLevel |
| 2320 | CAD ~ Intercept.High + Sex + APOB + RBP4 + SERPINA12 + LowLevel |
| 2321 | CAD ~ Intercept.High + Sex + APOB + RBP4 + SERPINA12 + LowLevel |
| 2322 | CAD ~ Intercept.High + Sex + APOB + RBP4 + SERPINA12 + LowLevel |
| 2323 | CAD ~ Intercept.High + Sex + APOB + RBP4 + SERPINA12 + LowLevel |
| 2324 | CAD ~ Intercept.High + Sex + APOB + RBP4 + LowLevel |
| 2325 | CAD ~ Intercept.High + Sex + APOB + RBP4 + LowLevel |
| 2326 | CAD ~ Intercept.High + Sex + APOB + RBP4 + LowLevel |
| 2327 | CAD ~ Intercept.High + Sex + APOB + RBP4 + LowLevel |
| 2328 | CAD ~ Intercept.High + Sex + APOB + RBP4 + LowLevel |
| 2329 | CAD ~ Intercept.High + Sex + APOB + RBP4 + LowLevel |
| 2330 | CAD ~ Intercept.High + Sex + APOB + RBP4 + LowLevel |
| 2331 | CAD ~ Intercept.High + Sex + APOB + RBP4 + LowLevel |
| 2332 | CAD ~ Intercept.High + Sex + APOB + RBP4 + LowLevel |
| 2333 | CAD ~ Intercept.High + Sex + APOB + RBP4 + LowLevel |
| 2334 | CAD ~ Intercept.High + Sex + APOB + SERPINA12 + LowLevel |
| 2335 | CAD ~ Intercept.High + Sex + APOB + SERPINA12 + LowLevel |
| 2336 | CAD ~ Intercept.High + Sex + APOB + SERPINA12 + LowLevel |
| 2337 | CAD ~ Intercept.High + Sex + APOB + SERPINA12 + LowLevel |
| 2338 | CAD ~ Intercept.High + Sex + APOB + SERPINA12 + LowLevel |
| 2339 | CAD ~ Intercept.High + Sex + APOB + SERPINA12 + LowLevel |
| 2340 | CAD ~ Intercept.High + Sex + APOB + SERPINA12 + LowLevel |
| 2341 | CAD ~ Intercept.High + Sex + APOB + SERPINA12 + LowLevel |
| 2342 | CAD ~ Intercept.High + Sex + APOB + SERPINA12 + LowLevel |
| 2343 | CAD ~ Intercept.High + Sex + APOB + SERPINA12 + LowLevel |
| 2344 | CAD ~ Intercept.High + Sex + APOB + LowLevel |
| 2345 | CAD ~ Intercept.High + Sex + APOB + LowLevel |
| 2346 | CAD ~ Intercept.High + Sex + APOB + LowLevel |
| 2347 | CAD ~ Intercept.High + Sex + APOB + LowLevel |
| 2348 | CAD ~ Intercept.High + Sex + APOB + LowLevel |
| 2349 | CAD ~ Intercept.High + Sex + corin + HSP70 + RBP4 + SERPINA12 + LowLevel |
| 2350 | CAD ~ Intercept.High + Sex + corin + HSP70 + RBP4 + SERPINA12 + LowLevel |
| 2351 | CAD ~ Intercept.High + Sex + corin + HSP70 + RBP4 + SERPINA12 + LowLevel |
| 2352 | CAD ~ Intercept.High + Sex + corin + HSP70 + RBP4 + SERPINA12 + LowLevel |
| 2353 | CAD ~ Intercept.High + Sex + corin + HSP70 + RBP4 + SERPINA12 + LowLevel |
| 2354 | CAD ~ Intercept.High + Sex + corin + HSP70 + RBP4 + LowLevel |
| 2355 | CAD ~ Intercept.High + Sex + corin + HSP70 + RBP4 + LowLevel |
| 2356 | CAD ~ Intercept.High + Sex + corin + HSP70 + RBP4 + LowLevel |
| 2357 | CAD ~ Intercept.High + Sex + corin + HSP70 + RBP4 + LowLevel |
| 2358 | CAD ~ Intercept.High + Sex + corin + HSP70 + RBP4 + LowLevel |
| 2359 | CAD ~ Intercept.High + Sex + corin + HSP70 + RBP4 + LowLevel |
| 2360 | CAD ~ Intercept.High + Sex + corin + HSP70 + RBP4 + LowLevel |
| 2361 | CAD ~ Intercept.High + Sex + corin + HSP70 + RBP4 + LowLevel |
| 2362 | CAD ~ Intercept.High + Sex + corin + HSP70 + RBP4 + LowLevel |
| 2363 | CAD ~ Intercept.High + Sex + corin + HSP70 + RBP4 + LowLevel |
| 2364 | CAD ~ Intercept.High + Sex + corin + HSP70 + SERPINA12 + LowLevel |
| 2365 | CAD ~ Intercept.High + Sex + corin + HSP70 + SERPINA12 + LowLevel |
| 2366 | CAD ~ Intercept.High + Sex + corin + HSP70 + SERPINA12 + LowLevel |
| 2367 | CAD ~ Intercept.High + Sex + corin + HSP70 + SERPINA12 + LowLevel |
| 2368 | CAD ~ Intercept.High + Sex + corin + HSP70 + SERPINA12 + LowLevel |
| 2369 | CAD ~ Intercept.High + Sex + corin + HSP70 + SERPINA12 + LowLevel |
| 2370 | CAD ~ Intercept.High + Sex + corin + HSP70 + SERPINA12 + LowLevel |
| 2371 | CAD ~ Intercept.High + Sex + corin + HSP70 + SERPINA12 + LowLevel |
| 2372 | CAD ~ Intercept.High + Sex + corin + HSP70 + SERPINA12 + LowLevel |
| 2373 | CAD ~ Intercept.High + Sex + corin + HSP70 + SERPINA12 + LowLevel |
| 2374 | CAD ~ Intercept.High + Sex + corin + HSP70 + LowLevel |
| 2375 | CAD ~ Intercept.High + Sex + corin + HSP70 + LowLevel |
| 2376 | CAD ~ Intercept.High + Sex + corin + HSP70 + LowLevel |
| 2377 | CAD ~ Intercept.High + Sex + corin + HSP70 + LowLevel |
| 2378 | CAD ~ Intercept.High + Sex + corin + HSP70 + LowLevel |
| 2379 | CAD ~ Intercept.High + Sex + corin + HSP70 + LowLevel |
| 2380 | CAD ~ Intercept.High + Sex + corin + HSP70 + LowLevel |
| 2381 | CAD ~ Intercept.High + Sex + corin + HSP70 + LowLevel |
| 2382 | CAD ~ Intercept.High + Sex + corin + HSP70 + LowLevel |
| 2383 | CAD ~ Intercept.High + Sex + corin + HSP70 + LowLevel |
| 2384 | CAD ~ Intercept.High + Sex + corin + RBP4 + SERPINA12 + LowLevel |
| 2385 | CAD ~ Intercept.High + Sex + corin + RBP4 + SERPINA12 + LowLevel |
| 2386 | CAD ~ Intercept.High + Sex + corin + RBP4 + SERPINA12 + LowLevel |
| 2387 | CAD ~ Intercept.High + Sex + corin + RBP4 + SERPINA12 + LowLevel |
| 2388 | CAD ~ Intercept.High + Sex + corin + RBP4 + SERPINA12 + LowLevel |
| 2389 | CAD ~ Intercept.High + Sex + corin + RBP4 + SERPINA12 + LowLevel |
| 2390 | CAD ~ Intercept.High + Sex + corin + RBP4 + SERPINA12 + LowLevel |
| 2391 | CAD ~ Intercept.High + Sex + corin + RBP4 + SERPINA12 + LowLevel |
| 2392 | CAD ~ Intercept.High + Sex + corin + RBP4 + SERPINA12 + LowLevel |
| 2393 | CAD ~ Intercept.High + Sex + corin + RBP4 + SERPINA12 + LowLevel |
| 2394 | CAD ~ Intercept.High + Sex + corin + RBP4 + LowLevel |
| 2395 | CAD ~ Intercept.High + Sex + corin + RBP4 + LowLevel |
| 2396 | CAD ~ Intercept.High + Sex + corin + RBP4 + LowLevel |
| 2397 | CAD ~ Intercept.High + Sex + corin + RBP4 + LowLevel |
| 2398 | CAD ~ Intercept.High + Sex + corin + RBP4 + LowLevel |
| 2399 | CAD ~ Intercept.High + Sex + corin + RBP4 + LowLevel |
| 2400 | CAD ~ Intercept.High + Sex + corin + RBP4 + LowLevel |
| 2401 | CAD ~ Intercept.High + Sex + corin + RBP4 + LowLevel |
| 2402 | CAD ~ Intercept.High + Sex + corin + RBP4 + LowLevel |
| 2403 | CAD ~ Intercept.High + Sex + corin + RBP4 + LowLevel |
| 2404 | CAD ~ Intercept.High + Sex + corin + SERPINA12 + LowLevel |
| 2405 | CAD ~ Intercept.High + Sex + corin + SERPINA12 + LowLevel |
| 2406 | CAD ~ Intercept.High + Sex + corin + SERPINA12 + LowLevel |
| 2407 | CAD ~ Intercept.High + Sex + corin + SERPINA12 + LowLevel |
| 2408 | CAD ~ Intercept.High + Sex + corin + SERPINA12 + LowLevel |
| 2409 | CAD ~ Intercept.High + Sex + corin + SERPINA12 + LowLevel |
| 2410 | CAD ~ Intercept.High + Sex + corin + SERPINA12 + LowLevel |
| 2411 | CAD ~ Intercept.High + Sex + corin + SERPINA12 + LowLevel |
| 2412 | CAD ~ Intercept.High + Sex + corin + SERPINA12 + LowLevel |
| 2413 | CAD ~ Intercept.High + Sex + corin + SERPINA12 + LowLevel |
| 2414 | CAD ~ Intercept.High + Sex + corin + LowLevel |
| 2415 | CAD ~ Intercept.High + Sex + corin + LowLevel |
| 2416 | CAD ~ Intercept.High + Sex + corin + LowLevel |
| 2417 | CAD ~ Intercept.High + Sex + corin + LowLevel |
| 2418 | CAD ~ Intercept.High + Sex + corin + LowLevel |
| 2419 | CAD ~ Intercept.High + Sex + HSP70 + RBP4 + SERPINA12 + LowLevel |
| 2420 | CAD ~ Intercept.High + Sex + HSP70 + RBP4 + SERPINA12 + LowLevel |
| 2421 | CAD ~ Intercept.High + Sex + HSP70 + RBP4 + SERPINA12 + LowLevel |
| 2422 | CAD ~ Intercept.High + Sex + HSP70 + RBP4 + SERPINA12 + LowLevel |
| 2423 | CAD ~ Intercept.High + Sex + HSP70 + RBP4 + SERPINA12 + LowLevel |
| 2424 | CAD ~ Intercept.High + Sex + HSP70 + RBP4 + SERPINA12 + LowLevel |
| 2425 | CAD ~ Intercept.High + Sex + HSP70 + RBP4 + SERPINA12 + LowLevel |
| 2426 | CAD ~ Intercept.High + Sex + HSP70 + RBP4 + SERPINA12 + LowLevel |
| 2427 | CAD ~ Intercept.High + Sex + HSP70 + RBP4 + SERPINA12 + LowLevel |
| 2428 | CAD ~ Intercept.High + Sex + HSP70 + RBP4 + SERPINA12 + LowLevel |
| 2429 | CAD ~ Intercept.High + Sex + HSP70 + RBP4 + LowLevel |
| 2430 | CAD ~ Intercept.High + Sex + HSP70 + RBP4 + LowLevel |
| 2431 | CAD ~ Intercept.High + Sex + HSP70 + RBP4 + LowLevel |
| 2432 | CAD ~ Intercept.High + Sex + HSP70 + RBP4 + LowLevel |
| 2433 | CAD ~ Intercept.High + Sex + HSP70 + RBP4 + LowLevel |
| 2434 | CAD ~ Intercept.High + Sex + HSP70 + RBP4 + LowLevel |
| 2435 | CAD ~ Intercept.High + Sex + HSP70 + RBP4 + LowLevel |
| 2436 | CAD ~ Intercept.High + Sex + HSP70 + RBP4 + LowLevel |
| 2437 | CAD ~ Intercept.High + Sex + HSP70 + RBP4 + LowLevel |
| 2438 | CAD ~ Intercept.High + Sex + HSP70 + RBP4 + LowLevel |
| 2439 | CAD ~ Intercept.High + Sex + HSP70 + SERPINA12 + LowLevel |
| 2440 | CAD ~ Intercept.High + Sex + HSP70 + SERPINA12 + LowLevel |
| 2441 | CAD ~ Intercept.High + Sex + HSP70 + SERPINA12 + LowLevel |
| 2442 | CAD ~ Intercept.High + Sex + HSP70 + SERPINA12 + LowLevel |
| 2443 | CAD ~ Intercept.High + Sex + HSP70 + SERPINA12 + LowLevel |
| 2444 | CAD ~ Intercept.High + Sex + HSP70 + SERPINA12 + LowLevel |
| 2445 | CAD ~ Intercept.High + Sex + HSP70 + SERPINA12 + LowLevel |
| 2446 | CAD ~ Intercept.High + Sex + HSP70 + SERPINA12 + LowLevel |
| 2447 | CAD ~ Intercept.High + Sex + HSP70 + SERPINA12 + LowLevel |
| 2448 | CAD ~ Intercept.High + Sex + HSP70 + SERPINA12 + LowLevel |
| 2449 | CAD ~ Intercept.High + Sex + HSP70 + LowLevel |
| 2450 | CAD ~ Intercept.High + Sex + HSP70 + LowLevel |
| 2451 | CAD ~ Intercept.High + Sex + HSP70 + LowLevel |
| 2452 | CAD ~ Intercept.High + Sex + HSP70 + LowLevel |
| 2453 | CAD ~ Intercept.High + Sex + HSP70 + LowLevel |
| 2454 | CAD ~ Intercept.High + Sex + RBP4 + SERPINA12 + LowLevel |
| 2455 | CAD ~ Intercept.High + Sex + RBP4 + SERPINA12 + LowLevel |
| 2456 | CAD ~ Intercept.High + Sex + RBP4 + SERPINA12 + LowLevel |
| 2457 | CAD ~ Intercept.High + Sex + RBP4 + SERPINA12 + LowLevel |
| 2458 | CAD ~ Intercept.High + Sex + RBP4 + SERPINA12 + LowLevel |
| 2459 | CAD ~ Intercept.High + Sex + RBP4 + SERPINA12 + LowLevel |
| 2460 | CAD ~ Intercept.High + Sex + RBP4 + SERPINA12 + LowLevel |
| 2461 | CAD ~ Intercept.High + Sex + RBP4 + SERPINA12 + LowLevel |
| 2462 | CAD ~ Intercept.High + Sex + RBP4 + SERPINA12 + LowLevel |
| 2463 | CAD ~ Intercept.High + Sex + RBP4 + SERPINA12 + LowLevel |
| 2464 | CAD ~ Intercept.High + Sex + RBP4 + LowLevel |
| 2465 | CAD ~ Intercept.High + Sex + RBP4 + LowLevel |
| 2466 | CAD ~ Intercept.High + Sex + RBP4 + LowLevel |
| 2467 | CAD ~ Intercept.High + Sex + RBP4 + LowLevel |
| 2468 | CAD ~ Intercept.High + Sex + RBP4 + LowLevel |
| 2469 | CAD ~ Intercept.High + Sex + SERPINA12 + LowLevel |
| 2470 | CAD ~ Intercept.High + Sex + SERPINA12 + LowLevel |
| 2471 | CAD ~ Intercept.High + Sex + SERPINA12 + LowLevel |
| 2472 | CAD ~ Intercept.High + Sex + SERPINA12 + LowLevel |
| 2473 | CAD ~ Intercept.High + Sex + SERPINA12 + LowLevel |
| 2474 | CAD ~ Intercept.High + Sex + LowLevel |
| 2475 | CAD ~ Intercept.High + HSP70 + RBP4 + SERPINA12 + LowLevel |
| 2476 | CAD ~ Intercept.High + HSP70 + RBP4 + SERPINA12 + LowLevel |
| 2477 | CAD ~ Intercept.High + HSP70 + RBP4 + SERPINA12 + LowLevel |
| 2478 | CAD ~ Intercept.High + HSP70 + RBP4 + SERPINA12 + LowLevel |
| 2479 | CAD ~ Intercept.High + HSP70 + RBP4 + SERPINA12 + LowLevel |
| 2480 | CAD ~ Intercept.High + HSP70 + RBP4 + SERPINA12 + LowLevel |
| 2481 | CAD ~ Intercept.High + HSP70 + RBP4 + SERPINA12 + LowLevel |
| 2482 | CAD ~ Intercept.High + HSP70 + RBP4 + SERPINA12 + LowLevel |
| 2483 | CAD ~ Intercept.High + HSP70 + RBP4 + SERPINA12 + LowLevel |
| 2484 | CAD ~ Intercept.High + HSP70 + RBP4 + SERPINA12 + LowLevel |
| 2485 | CAD ~ Intercept.High + HSP70 + RBP4 + LowLevel |
| 2486 | CAD ~ Intercept.High + HSP70 + RBP4 + LowLevel |
| 2487 | CAD ~ Intercept.High + HSP70 + RBP4 + LowLevel |
| 2488 | CAD ~ Intercept.High + HSP70 + RBP4 + LowLevel |
| 2489 | CAD ~ Intercept.High + HSP70 + RBP4 + LowLevel |
| 2490 | CAD ~ Intercept.High + HSP70 + SERPINA12 + LowLevel |
| 2491 | CAD ~ Intercept.High + HSP70 + SERPINA12 + LowLevel |
| 2492 | CAD ~ Intercept.High + HSP70 + SERPINA12 + LowLevel |
| 2493 | CAD ~ Intercept.High + HSP70 + SERPINA12 + LowLevel |
| 2494 | CAD ~ Intercept.High + HSP70 + SERPINA12 + LowLevel |
| 2495 | CAD ~ Intercept.High + HSP70 + LowLevel |
| 2496 | CAD ~ Intercept.High + RBP4 + SERPINA12 + LowLevel |
| 2497 | CAD ~ Intercept.High + RBP4 + SERPINA12 + LowLevel |
| 2498 | CAD ~ Intercept.High + RBP4 + SERPINA12 + LowLevel |
| 2499 | CAD ~ Intercept.High + RBP4 + SERPINA12 + LowLevel |
| 2500 | CAD ~ Intercept.High + RBP4 + SERPINA12 + LowLevel |
| 2501 | CAD ~ Intercept.High + RBP4 + LowLevel |
| 2502 | CAD ~ Intercept.High + SERPINA12 + LowLevel |
| 2503 | CAD ~ Intercept.High + Age + APOB + corin + HSP70 + LowLevel |
| 2504 | CAD ~ Intercept.High + Age + APOB + corin + HSP70 + LowLevel |
| 2505 | CAD ~ Intercept.High + Age + APOB + corin + HSP70 + LowLevel |
| 2506 | CAD ~ Intercept.High + Age + APOB + corin + HSP70 + LowLevel |
| 2507 | CAD ~ Intercept.High + Age + APOB + corin + HSP70 + LowLevel |
| 2508 | CAD ~ Intercept.High + Age + APOB + corin + RBP4 + LowLevel |
| 2509 | CAD ~ Intercept.High + Age + APOB + corin + RBP4 + LowLevel |
| 2510 | CAD ~ Intercept.High + Age + APOB + corin + RBP4 + LowLevel |
| 2511 | CAD ~ Intercept.High + Age + APOB + corin + RBP4 + LowLevel |
| 2512 | CAD ~ Intercept.High + Age + APOB + corin + RBP4 + LowLevel |
| 2513 | CAD ~ Intercept.High + Age + APOB + corin + SERPINA12 + LowLevel |
| 2514 | CAD ~ Intercept.High + Age + APOB + corin + SERPINA12 + LowLevel |
| 2515 | CAD ~ Intercept.High + Age + APOB + corin + SERPINA12 + LowLevel |
| 2516 | CAD ~ Intercept.High + Age + APOB + corin + SERPINA12 + LowLevel |
| 2517 | CAD ~ Intercept.High + Age + APOB + corin + SERPINA12 + LowLevel |
| 2518 | CAD ~ Intercept.High + Age + APOB + corin + LowLevel |
| 2519 | CAD ~ Intercept.High + Age + APOB + corin + LowLevel |
| 2520 | CAD ~ Intercept.High + Age + APOB + corin + LowLevel |
| 2521 | CAD ~ Intercept.High + Age + APOB + corin + LowLevel |
| 2522 | CAD ~ Intercept.High + Age + APOB + corin + LowLevel |
| 2523 | CAD ~ Intercept.High + Age + APOB + corin + LowLevel |
| 2524 | CAD ~ Intercept.High + Age + APOB + corin + LowLevel |
| 2525 | CAD ~ Intercept.High + Age + APOB + corin + LowLevel |
| 2526 | CAD ~ Intercept.High + Age + APOB + corin + LowLevel |
| 2527 | CAD ~ Intercept.High + Age + APOB + corin + LowLevel |
| 2528 | CAD ~ Intercept.High + Age + APOB + HSP70 + RBP4 + LowLevel |
| 2529 | CAD ~ Intercept.High + Age + APOB + HSP70 + RBP4 + LowLevel |
| 2530 | CAD ~ Intercept.High + Age + APOB + HSP70 + RBP4 + LowLevel |
| 2531 | CAD ~ Intercept.High + Age + APOB + HSP70 + RBP4 + LowLevel |
| 2532 | CAD ~ Intercept.High + Age + APOB + HSP70 + RBP4 + LowLevel |
| 2533 | CAD ~ Intercept.High + Age + APOB + HSP70 + SERPINA12 + LowLevel |
| 2534 | CAD ~ Intercept.High + Age + APOB + HSP70 + SERPINA12 + LowLevel |
| 2535 | CAD ~ Intercept.High + Age + APOB + HSP70 + SERPINA12 + LowLevel |
| 2536 | CAD ~ Intercept.High + Age + APOB + HSP70 + SERPINA12 + LowLevel |
| 2537 | CAD ~ Intercept.High + Age + APOB + HSP70 + SERPINA12 + LowLevel |
| 2538 | CAD ~ Intercept.High + Age + APOB + HSP70 + LowLevel |
| 2539 | CAD ~ Intercept.High + Age + APOB + HSP70 + LowLevel |
| 2540 | CAD ~ Intercept.High + Age + APOB + HSP70 + LowLevel |
| 2541 | CAD ~ Intercept.High + Age + APOB + HSP70 + LowLevel |
| 2542 | CAD ~ Intercept.High + Age + APOB + HSP70 + LowLevel |
| 2543 | CAD ~ Intercept.High + Age + APOB + HSP70 + LowLevel |
| 2544 | CAD ~ Intercept.High + Age + APOB + HSP70 + LowLevel |
| 2545 | CAD ~ Intercept.High + Age + APOB + HSP70 + LowLevel |
| 2546 | CAD ~ Intercept.High + Age + APOB + HSP70 + LowLevel |
| 2547 | CAD ~ Intercept.High + Age + APOB + HSP70 + LowLevel |
| 2548 | CAD ~ Intercept.High + Age + APOB + RBP4 + SERPINA12 + LowLevel |
| 2549 | CAD ~ Intercept.High + Age + APOB + RBP4 + SERPINA12 + LowLevel |
| 2550 | CAD ~ Intercept.High + Age + APOB + RBP4 + SERPINA12 + LowLevel |
| 2551 | CAD ~ Intercept.High + Age + APOB + RBP4 + SERPINA12 + LowLevel |
| 2552 | CAD ~ Intercept.High + Age + APOB + RBP4 + SERPINA12 + LowLevel |
| 2553 | CAD ~ Intercept.High + Age + APOB + RBP4 + LowLevel |
| 2554 | CAD ~ Intercept.High + Age + APOB + RBP4 + LowLevel |
| 2555 | CAD ~ Intercept.High + Age + APOB + RBP4 + LowLevel |
| 2556 | CAD ~ Intercept.High + Age + APOB + RBP4 + LowLevel |
| 2557 | CAD ~ Intercept.High + Age + APOB + RBP4 + LowLevel |
| 2558 | CAD ~ Intercept.High + Age + APOB + RBP4 + LowLevel |
| 2559 | CAD ~ Intercept.High + Age + APOB + RBP4 + LowLevel |
| 2560 | CAD ~ Intercept.High + Age + APOB + RBP4 + LowLevel |
| 2561 | CAD ~ Intercept.High + Age + APOB + RBP4 + LowLevel |
| 2562 | CAD ~ Intercept.High + Age + APOB + RBP4 + LowLevel |
| 2563 | CAD ~ Intercept.High + Age + APOB + SERPINA12 + LowLevel |
| 2564 | CAD ~ Intercept.High + Age + APOB + SERPINA12 + LowLevel |
| 2565 | CAD ~ Intercept.High + Age + APOB + SERPINA12 + LowLevel |
| 2566 | CAD ~ Intercept.High + Age + APOB + SERPINA12 + LowLevel |
| 2567 | CAD ~ Intercept.High + Age + APOB + SERPINA12 + LowLevel |
| 2568 | CAD ~ Intercept.High + Age + APOB + SERPINA12 + LowLevel |
| 2569 | CAD ~ Intercept.High + Age + APOB + SERPINA12 + LowLevel |
| 2570 | CAD ~ Intercept.High + Age + APOB + SERPINA12 + LowLevel |
| 2571 | CAD ~ Intercept.High + Age + APOB + SERPINA12 + LowLevel |
| 2572 | CAD ~ Intercept.High + Age + APOB + SERPINA12 + LowLevel |
| 2573 | CAD ~ Intercept.High + Age + APOB + LowLevel |
| 2574 | CAD ~ Intercept.High + Age + APOB + LowLevel |
| 2575 | CAD ~ Intercept.High + Age + APOB + LowLevel |
| 2576 | CAD ~ Intercept.High + Age + APOB + LowLevel |
| 2577 | CAD ~ Intercept.High + Age + APOB + LowLevel |
| 2578 | CAD ~ Intercept.High + Age + APOB + LowLevel |
| 2579 | CAD ~ Intercept.High + Age + APOB + LowLevel |
| 2580 | CAD ~ Intercept.High + Age + APOB + LowLevel |
| 2581 | CAD ~ Intercept.High + Age + APOB + LowLevel |
| 2582 | CAD ~ Intercept.High + Age + APOB + LowLevel |
| 2583 | CAD ~ Intercept.High + Age + corin + HSP70 + RBP4 + LowLevel |
| 2584 | CAD ~ Intercept.High + Age + corin + HSP70 + RBP4 + LowLevel |
| 2585 | CAD ~ Intercept.High + Age + corin + HSP70 + RBP4 + LowLevel |
| 2586 | CAD ~ Intercept.High + Age + corin + HSP70 + RBP4 + LowLevel |
| 2587 | CAD ~ Intercept.High + Age + corin + HSP70 + RBP4 + LowLevel |
| 2588 | CAD ~ Intercept.High + Age + corin + HSP70 + SERPINA12 + LowLevel |
| 2589 | CAD ~ Intercept.High + Age + corin + HSP70 + SERPINA12 + LowLevel |
| 2590 | CAD ~ Intercept.High + Age + corin + HSP70 + SERPINA12 + LowLevel |
| 2591 | CAD ~ Intercept.High + Age + corin + HSP70 + SERPINA12 + LowLevel |
| 2592 | CAD ~ Intercept.High + Age + corin + HSP70 + SERPINA12 + LowLevel |
| 2593 | CAD ~ Intercept.High + Age + corin + HSP70 + LowLevel |
| 2594 | CAD ~ Intercept.High + Age + corin + HSP70 + LowLevel |
| 2595 | CAD ~ Intercept.High + Age + corin + HSP70 + LowLevel |
| 2596 | CAD ~ Intercept.High + Age + corin + HSP70 + LowLevel |
| 2597 | CAD ~ Intercept.High + Age + corin + HSP70 + LowLevel |
| 2598 | CAD ~ Intercept.High + Age + corin + HSP70 + LowLevel |
| 2599 | CAD ~ Intercept.High + Age + corin + HSP70 + LowLevel |
| 2600 | CAD ~ Intercept.High + Age + corin + HSP70 + LowLevel |
| 2601 | CAD ~ Intercept.High + Age + corin + HSP70 + LowLevel |
| 2602 | CAD ~ Intercept.High + Age + corin + HSP70 + LowLevel |
| 2603 | CAD ~ Intercept.High + Age + corin + RBP4 + SERPINA12 + LowLevel |
| 2604 | CAD ~ Intercept.High + Age + corin + RBP4 + SERPINA12 + LowLevel |
| 2605 | CAD ~ Intercept.High + Age + corin + RBP4 + SERPINA12 + LowLevel |
| 2606 | CAD ~ Intercept.High + Age + corin + RBP4 + SERPINA12 + LowLevel |
| 2607 | CAD ~ Intercept.High + Age + corin + RBP4 + SERPINA12 + LowLevel |
| 2608 | CAD ~ Intercept.High + Age + corin + RBP4 + LowLevel |
| 2609 | CAD ~ Intercept.High + Age + corin + RBP4 + LowLevel |
| 2610 | CAD ~ Intercept.High + Age + corin + RBP4 + LowLevel |
| 2611 | CAD ~ Intercept.High + Age + corin + RBP4 + LowLevel |
| 2612 | CAD ~ Intercept.High + Age + corin + RBP4 + LowLevel |
| 2613 | CAD ~ Intercept.High + Age + corin + RBP4 + LowLevel |
| 2614 | CAD ~ Intercept.High + Age + corin + RBP4 + LowLevel |
| 2615 | CAD ~ Intercept.High + Age + corin + RBP4 + LowLevel |
| 2616 | CAD ~ Intercept.High + Age + corin + RBP4 + LowLevel |
| 2617 | CAD ~ Intercept.High + Age + corin + RBP4 + LowLevel |
| 2618 | CAD ~ Intercept.High + Age + corin + SERPINA12 + LowLevel |
| 2619 | CAD ~ Intercept.High + Age + corin + SERPINA12 + LowLevel |
| 2620 | CAD ~ Intercept.High + Age + corin + SERPINA12 + LowLevel |
| 2621 | CAD ~ Intercept.High + Age + corin + SERPINA12 + LowLevel |
| 2622 | CAD ~ Intercept.High + Age + corin + SERPINA12 + LowLevel |
| 2623 | CAD ~ Intercept.High + Age + corin + SERPINA12 + LowLevel |
| 2624 | CAD ~ Intercept.High + Age + corin + SERPINA12 + LowLevel |
| 2625 | CAD ~ Intercept.High + Age + corin + SERPINA12 + LowLevel |
| 2626 | CAD ~ Intercept.High + Age + corin + SERPINA12 + LowLevel |
| 2627 | CAD ~ Intercept.High + Age + corin + SERPINA12 + LowLevel |
| 2628 | CAD ~ Intercept.High + Age + corin + LowLevel |
| 2629 | CAD ~ Intercept.High + Age + corin + LowLevel |
| 2630 | CAD ~ Intercept.High + Age + corin + LowLevel |
| 2631 | CAD ~ Intercept.High + Age + corin + LowLevel |
| 2632 | CAD ~ Intercept.High + Age + corin + LowLevel |
| 2633 | CAD ~ Intercept.High + Age + corin + LowLevel |
| 2634 | CAD ~ Intercept.High + Age + corin + LowLevel |
| 2635 | CAD ~ Intercept.High + Age + corin + LowLevel |
| 2636 | CAD ~ Intercept.High + Age + corin + LowLevel |
| 2637 | CAD ~ Intercept.High + Age + corin + LowLevel |
| 2638 | CAD ~ Intercept.High + Age + HSP70 + RBP4 + SERPINA12 + LowLevel |
| 2639 | CAD ~ Intercept.High + Age + HSP70 + RBP4 + SERPINA12 + LowLevel |
| 2640 | CAD ~ Intercept.High + Age + HSP70 + RBP4 + SERPINA12 + LowLevel |
| 2641 | CAD ~ Intercept.High + Age + HSP70 + RBP4 + SERPINA12 + LowLevel |
| 2642 | CAD ~ Intercept.High + Age + HSP70 + RBP4 + SERPINA12 + LowLevel |
| 2643 | CAD ~ Intercept.High + Age + HSP70 + RBP4 + LowLevel |
| 2644 | CAD ~ Intercept.High + Age + HSP70 + RBP4 + LowLevel |
| 2645 | CAD ~ Intercept.High + Age + HSP70 + RBP4 + LowLevel |
| 2646 | CAD ~ Intercept.High + Age + HSP70 + RBP4 + LowLevel |
| 2647 | CAD ~ Intercept.High + Age + HSP70 + RBP4 + LowLevel |
| 2648 | CAD ~ Intercept.High + Age + HSP70 + RBP4 + LowLevel |
| 2649 | CAD ~ Intercept.High + Age + HSP70 + RBP4 + LowLevel |
| 2650 | CAD ~ Intercept.High + Age + HSP70 + RBP4 + LowLevel |
| 2651 | CAD ~ Intercept.High + Age + HSP70 + RBP4 + LowLevel |
| 2652 | CAD ~ Intercept.High + Age + HSP70 + RBP4 + LowLevel |
| 2653 | CAD ~ Intercept.High + Age + HSP70 + SERPINA12 + LowLevel |
| 2654 | CAD ~ Intercept.High + Age + HSP70 + SERPINA12 + LowLevel |
| 2655 | CAD ~ Intercept.High + Age + HSP70 + SERPINA12 + LowLevel |
| 2656 | CAD ~ Intercept.High + Age + HSP70 + SERPINA12 + LowLevel |
| 2657 | CAD ~ Intercept.High + Age + HSP70 + SERPINA12 + LowLevel |
| 2658 | CAD ~ Intercept.High + Age + HSP70 + SERPINA12 + LowLevel |
| 2659 | CAD ~ Intercept.High + Age + HSP70 + SERPINA12 + LowLevel |
| 2660 | CAD ~ Intercept.High + Age + HSP70 + SERPINA12 + LowLevel |
| 2661 | CAD ~ Intercept.High + Age + HSP70 + SERPINA12 + LowLevel |
| 2662 | CAD ~ Intercept.High + Age + HSP70 + SERPINA12 + LowLevel |
| 2663 | CAD ~ Intercept.High + Age + HSP70 + LowLevel |
| 2664 | CAD ~ Intercept.High + Age + HSP70 + LowLevel |
| 2665 | CAD ~ Intercept.High + Age + HSP70 + LowLevel |
| 2666 | CAD ~ Intercept.High + Age + HSP70 + LowLevel |
| 2667 | CAD ~ Intercept.High + Age + HSP70 + LowLevel |
| 2668 | CAD ~ Intercept.High + Age + HSP70 + LowLevel |
| 2669 | CAD ~ Intercept.High + Age + HSP70 + LowLevel |
| 2670 | CAD ~ Intercept.High + Age + HSP70 + LowLevel |
| 2671 | CAD ~ Intercept.High + Age + HSP70 + LowLevel |
| 2672 | CAD ~ Intercept.High + Age + HSP70 + LowLevel |
| 2673 | CAD ~ Intercept.High + Age + RBP4 + SERPINA12 + LowLevel |
| 2674 | CAD ~ Intercept.High + Age + RBP4 + SERPINA12 + LowLevel |
| 2675 | CAD ~ Intercept.High + Age + RBP4 + SERPINA12 + LowLevel |
| 2676 | CAD ~ Intercept.High + Age + RBP4 + SERPINA12 + LowLevel |
| 2677 | CAD ~ Intercept.High + Age + RBP4 + SERPINA12 + LowLevel |
| 2678 | CAD ~ Intercept.High + Age + RBP4 + SERPINA12 + LowLevel |
| 2679 | CAD ~ Intercept.High + Age + RBP4 + SERPINA12 + LowLevel |
| 2680 | CAD ~ Intercept.High + Age + RBP4 + SERPINA12 + LowLevel |
| 2681 | CAD ~ Intercept.High + Age + RBP4 + SERPINA12 + LowLevel |
| 2682 | CAD ~ Intercept.High + Age + RBP4 + SERPINA12 + LowLevel |
| 2683 | CAD ~ Intercept.High + Age + RBP4 + LowLevel |
| 2684 | CAD ~ Intercept.High + Age + RBP4 + LowLevel |
| 2685 | CAD ~ Intercept.High + Age + RBP4 + LowLevel |
| 2686 | CAD ~ Intercept.High + Age + RBP4 + LowLevel |
| 2687 | CAD ~ Intercept.High + Age + RBP4 + LowLevel |
| 2688 | CAD ~ Intercept.High + Age + RBP4 + LowLevel |
| 2689 | CAD ~ Intercept.High + Age + RBP4 + LowLevel |
| 2690 | CAD ~ Intercept.High + Age + RBP4 + LowLevel |
| 2691 | CAD ~ Intercept.High + Age + RBP4 + LowLevel |
| 2692 | CAD ~ Intercept.High + Age + RBP4 + LowLevel |
| 2693 | CAD ~ Intercept.High + Age + SERPINA12 + LowLevel |
| 2694 | CAD ~ Intercept.High + Age + SERPINA12 + LowLevel |
| 2695 | CAD ~ Intercept.High + Age + SERPINA12 + LowLevel |
| 2696 | CAD ~ Intercept.High + Age + SERPINA12 + LowLevel |
| 2697 | CAD ~ Intercept.High + Age + SERPINA12 + LowLevel |
| 2698 | CAD ~ Intercept.High + Age + SERPINA12 + LowLevel |
| 2699 | CAD ~ Intercept.High + Age + SERPINA12 + LowLevel |
| 2700 | CAD ~ Intercept.High + Age + SERPINA12 + LowLevel |
| 2701 | CAD ~ Intercept.High + Age + SERPINA12 + LowLevel |
| 2702 | CAD ~ Intercept.High + Age + SERPINA12 + LowLevel |
| 2703 | CAD ~ Intercept.High + Age + LowLevel |
| 2704 | CAD ~ Intercept.High + Age + LowLevel |
| 2705 | CAD ~ Intercept.High + Age + LowLevel |
| 2706 | CAD ~ Intercept.High + Age + LowLevel |
| 2707 | CAD ~ Intercept.High + Age + LowLevel |
| 2708 | CAD ~ Intercept.High + APOB + corin + HSP70 + RBP4 + LowLevel |
| 2709 | CAD ~ Intercept.High + APOB + corin + HSP70 + RBP4 + LowLevel |
| 2710 | CAD ~ Intercept.High + APOB + corin + HSP70 + RBP4 + LowLevel |
| 2711 | CAD ~ Intercept.High + APOB + corin + HSP70 + RBP4 + LowLevel |
| 2712 | CAD ~ Intercept.High + APOB + corin + HSP70 + RBP4 + LowLevel |
| 2713 | CAD ~ Intercept.High + APOB + corin + HSP70 + SERPINA12 + LowLevel |
| 2714 | CAD ~ Intercept.High + APOB + corin + HSP70 + SERPINA12 + LowLevel |
| 2715 | CAD ~ Intercept.High + APOB + corin + HSP70 + SERPINA12 + LowLevel |
| 2716 | CAD ~ Intercept.High + APOB + corin + HSP70 + SERPINA12 + LowLevel |
| 2717 | CAD ~ Intercept.High + APOB + corin + HSP70 + SERPINA12 + LowLevel |
| 2718 | CAD ~ Intercept.High + APOB + corin + HSP70 + LowLevel |
| 2719 | CAD ~ Intercept.High + APOB + corin + HSP70 + LowLevel |
| 2720 | CAD ~ Intercept.High + APOB + corin + HSP70 + LowLevel |
| 2721 | CAD ~ Intercept.High + APOB + corin + HSP70 + LowLevel |
| 2722 | CAD ~ Intercept.High + APOB + corin + HSP70 + LowLevel |
| 2723 | CAD ~ Intercept.High + APOB + corin + HSP70 + LowLevel |
| 2724 | CAD ~ Intercept.High + APOB + corin + HSP70 + LowLevel |
| 2725 | CAD ~ Intercept.High + APOB + corin + HSP70 + LowLevel |
| 2726 | CAD ~ Intercept.High + APOB + corin + HSP70 + LowLevel |
| 2727 | CAD ~ Intercept.High + APOB + corin + HSP70 + LowLevel |
| 2728 | CAD ~ Intercept.High + APOB + corin + RBP4 + SERPINA12 + LowLevel |
| 2729 | CAD ~ Intercept.High + APOB + corin + RBP4 + SERPINA12 + LowLevel |
| 2730 | CAD ~ Intercept.High + APOB + corin + RBP4 + SERPINA12 + LowLevel |
| 2731 | CAD ~ Intercept.High + APOB + corin + RBP4 + SERPINA12 + LowLevel |
| 2732 | CAD ~ Intercept.High + APOB + corin + RBP4 + SERPINA12 + LowLevel |
| 2733 | CAD ~ Intercept.High + APOB + corin + RBP4 + LowLevel |
| 2734 | CAD ~ Intercept.High + APOB + corin + RBP4 + LowLevel |
| 2735 | CAD ~ Intercept.High + APOB + corin + RBP4 + LowLevel |
| 2736 | CAD ~ Intercept.High + APOB + corin + RBP4 + LowLevel |
| 2737 | CAD ~ Intercept.High + APOB + corin + RBP4 + LowLevel |
| 2738 | CAD ~ Intercept.High + APOB + corin + RBP4 + LowLevel |
| 2739 | CAD ~ Intercept.High + APOB + corin + RBP4 + LowLevel |
| 2740 | CAD ~ Intercept.High + APOB + corin + RBP4 + LowLevel |
| 2741 | CAD ~ Intercept.High + APOB + corin + RBP4 + LowLevel |
| 2742 | CAD ~ Intercept.High + APOB + corin + RBP4 + LowLevel |
| 2743 | CAD ~ Intercept.High + APOB + corin + SERPINA12 + LowLevel |
| 2744 | CAD ~ Intercept.High + APOB + corin + SERPINA12 + LowLevel |
| 2745 | CAD ~ Intercept.High + APOB + corin + SERPINA12 + LowLevel |
| 2746 | CAD ~ Intercept.High + APOB + corin + SERPINA12 + LowLevel |
| 2747 | CAD ~ Intercept.High + APOB + corin + SERPINA12 + LowLevel |
| 2748 | CAD ~ Intercept.High + APOB + corin + SERPINA12 + LowLevel |
| 2749 | CAD ~ Intercept.High + APOB + corin + SERPINA12 + LowLevel |
| 2750 | CAD ~ Intercept.High + APOB + corin + SERPINA12 + LowLevel |
| 2751 | CAD ~ Intercept.High + APOB + corin + SERPINA12 + LowLevel |
| 2752 | CAD ~ Intercept.High + APOB + corin + SERPINA12 + LowLevel |
| 2753 | CAD ~ Intercept.High + APOB + corin + LowLevel |
| 2754 | CAD ~ Intercept.High + APOB + corin + LowLevel |
| 2755 | CAD ~ Intercept.High + APOB + corin + LowLevel |
| 2756 | CAD ~ Intercept.High + APOB + corin + LowLevel |
| 2757 | CAD ~ Intercept.High + APOB + corin + LowLevel |
| 2758 | CAD ~ Intercept.High + APOB + corin + LowLevel |
| 2759 | CAD ~ Intercept.High + APOB + corin + LowLevel |
| 2760 | CAD ~ Intercept.High + APOB + corin + LowLevel |
| 2761 | CAD ~ Intercept.High + APOB + corin + LowLevel |
| 2762 | CAD ~ Intercept.High + APOB + corin + LowLevel |
| 2763 | CAD ~ Intercept.High + APOB + HSP70 + RBP4 + SERPINA12 + LowLevel |
| 2764 | CAD ~ Intercept.High + APOB + HSP70 + RBP4 + SERPINA12 + LowLevel |
| 2765 | CAD ~ Intercept.High + APOB + HSP70 + RBP4 + SERPINA12 + LowLevel |
| 2766 | CAD ~ Intercept.High + APOB + HSP70 + RBP4 + SERPINA12 + LowLevel |
| 2767 | CAD ~ Intercept.High + APOB + HSP70 + RBP4 + SERPINA12 + LowLevel |
| 2768 | CAD ~ Intercept.High + APOB + HSP70 + RBP4 + LowLevel |
| 2769 | CAD ~ Intercept.High + APOB + HSP70 + RBP4 + LowLevel |
| 2770 | CAD ~ Intercept.High + APOB + HSP70 + RBP4 + LowLevel |
| 2771 | CAD ~ Intercept.High + APOB + HSP70 + RBP4 + LowLevel |
| 2772 | CAD ~ Intercept.High + APOB + HSP70 + RBP4 + LowLevel |
| 2773 | CAD ~ Intercept.High + APOB + HSP70 + RBP4 + LowLevel |
| 2774 | CAD ~ Intercept.High + APOB + HSP70 + RBP4 + LowLevel |
| 2775 | CAD ~ Intercept.High + APOB + HSP70 + RBP4 + LowLevel |
| 2776 | CAD ~ Intercept.High + APOB + HSP70 + RBP4 + LowLevel |
| 2777 | CAD ~ Intercept.High + APOB + HSP70 + RBP4 + LowLevel |
| 2778 | CAD ~ Intercept.High + APOB + HSP70 + SERPINA12 + LowLevel |
| 2779 | CAD ~ Intercept.High + APOB + HSP70 + SERPINA12 + LowLevel |
| 2780 | CAD ~ Intercept.High + APOB + HSP70 + SERPINA12 + LowLevel |
| 2781 | CAD ~ Intercept.High + APOB + HSP70 + SERPINA12 + LowLevel |
| 2782 | CAD ~ Intercept.High + APOB + HSP70 + SERPINA12 + LowLevel |
| 2783 | CAD ~ Intercept.High + APOB + HSP70 + SERPINA12 + LowLevel |
| 2784 | CAD ~ Intercept.High + APOB + HSP70 + SERPINA12 + LowLevel |
| 2785 | CAD ~ Intercept.High + APOB + HSP70 + SERPINA12 + LowLevel |
| 2786 | CAD ~ Intercept.High + APOB + HSP70 + SERPINA12 + LowLevel |
| 2787 | CAD ~ Intercept.High + APOB + HSP70 + SERPINA12 + LowLevel |
| 2788 | CAD ~ Intercept.High + APOB + HSP70 + LowLevel |
| 2789 | CAD ~ Intercept.High + APOB + HSP70 + LowLevel |
| 2790 | CAD ~ Intercept.High + APOB + HSP70 + LowLevel |
| 2791 | CAD ~ Intercept.High + APOB + HSP70 + LowLevel |
| 2792 | CAD ~ Intercept.High + APOB + HSP70 + LowLevel |
| 2793 | CAD ~ Intercept.High + APOB + HSP70 + LowLevel |
| 2794 | CAD ~ Intercept.High + APOB + HSP70 + LowLevel |
| 2795 | CAD ~ Intercept.High + APOB + HSP70 + LowLevel |
| 2796 | CAD ~ Intercept.High + APOB + HSP70 + LowLevel |
| 2797 | CAD ~ Intercept.High + APOB + HSP70 + LowLevel |
| 2798 | CAD ~ Intercept.High + APOB + RBP4 + SERPINA12 + LowLevel |
| 2799 | CAD ~ Intercept.High + APOB + RBP4 + SERPINA12 + LowLevel |
| 2800 | CAD ~ Intercept.High + APOB + RBP4 + SERPINA12 + LowLevel |
| 2801 | CAD ~ Intercept.High + APOB + RBP4 + SERPINA12 + LowLevel |
| 2802 | CAD ~ Intercept.High + APOB + RBP4 + SERPINA12 + LowLevel |
| 2803 | CAD ~ Intercept.High + APOB + RBP4 + SERPINA12 + LowLevel |
| 2804 | CAD ~ Intercept.High + APOB + RBP4 + SERPINA12 + LowLevel |
| 2805 | CAD ~ Intercept.High + APOB + RBP4 + SERPINA12 + LowLevel |
| 2806 | CAD ~ Intercept.High + APOB + RBP4 + SERPINA12 + LowLevel |
| 2807 | CAD ~ Intercept.High + APOB + RBP4 + SERPINA12 + LowLevel |
| 2808 | CAD ~ Intercept.High + APOB + RBP4 + LowLevel |
| 2809 | CAD ~ Intercept.High + APOB + RBP4 + LowLevel |
| 2810 | CAD ~ Intercept.High + APOB + RBP4 + LowLevel |
| 2811 | CAD ~ Intercept.High + APOB + RBP4 + LowLevel |
| 2812 | CAD ~ Intercept.High + APOB + RBP4 + LowLevel |
| 2813 | CAD ~ Intercept.High + APOB + RBP4 + LowLevel |
| 2814 | CAD ~ Intercept.High + APOB + RBP4 + LowLevel |
| 2815 | CAD ~ Intercept.High + APOB + RBP4 + LowLevel |
| 2816 | CAD ~ Intercept.High + APOB + RBP4 + LowLevel |
| 2817 | CAD ~ Intercept.High + APOB + RBP4 + LowLevel |
| 2818 | CAD ~ Intercept.High + APOB + SERPINA12 + LowLevel |
| 2819 | CAD ~ Intercept.High + APOB + SERPINA12 + LowLevel |
| 2820 | CAD ~ Intercept.High + APOB + SERPINA12 + LowLevel |
| 2821 | CAD ~ Intercept.High + APOB + SERPINA12 + LowLevel |
| 2822 | CAD ~ Intercept.High + APOB + SERPINA12 + LowLevel |
| 2823 | CAD ~ Intercept.High + APOB + SERPINA12 + LowLevel |
| 2824 | CAD ~ Intercept.High + APOB + SERPINA12 + LowLevel |
| 2825 | CAD ~ Intercept.High + APOB + SERPINA12 + LowLevel |
| 2826 | CAD ~ Intercept.High + APOB + SERPINA12 + LowLevel |
| 2827 | CAD ~ Intercept.High + APOB + SERPINA12 + LowLevel |
| 2828 | CAD ~ Intercept.High + APOB + LowLevel |
| 2829 | CAD ~ Intercept.High + APOB + LowLevel |
| 2830 | CAD ~ Intercept.High + APOB + LowLevel |
| 2831 | CAD ~ Intercept.High + APOB + LowLevel |
| 2832 | CAD ~ Intercept.High + APOB + LowLevel |
| 2833 | CAD ~ Intercept.High + corin + HSP70 + RBP4 + SERPINA12 + LowLevel |
| 2834 | CAD ~ Intercept.High + corin + HSP70 + RBP4 + SERPINA12 + LowLevel |
| 2835 | CAD ~ Intercept.High + corin + HSP70 + RBP4 + SERPINA12 + LowLevel |
| 2836 | CAD ~ Intercept.High + corin + HSP70 + RBP4 + SERPINA12 + LowLevel |
| 2837 | CAD ~ Intercept.High + corin + HSP70 + RBP4 + SERPINA12 + LowLevel |
| 2838 | CAD ~ Intercept.High + corin + HSP70 + RBP4 + LowLevel |
| 2839 | CAD ~ Intercept.High + corin + HSP70 + RBP4 + LowLevel |
| 2840 | CAD ~ Intercept.High + corin + HSP70 + RBP4 + LowLevel |
| 2841 | CAD ~ Intercept.High + corin + HSP70 + RBP4 + LowLevel |
| 2842 | CAD ~ Intercept.High + corin + HSP70 + RBP4 + LowLevel |
| 2843 | CAD ~ Intercept.High + corin + HSP70 + RBP4 + LowLevel |
| 2844 | CAD ~ Intercept.High + corin + HSP70 + RBP4 + LowLevel |
| 2845 | CAD ~ Intercept.High + corin + HSP70 + RBP4 + LowLevel |
| 2846 | CAD ~ Intercept.High + corin + HSP70 + RBP4 + LowLevel |
| 2847 | CAD ~ Intercept.High + corin + HSP70 + RBP4 + LowLevel |
| 2848 | CAD ~ Intercept.High + corin + HSP70 + SERPINA12 + LowLevel |
| 2849 | CAD ~ Intercept.High + corin + HSP70 + SERPINA12 + LowLevel |
| 2850 | CAD ~ Intercept.High + corin + HSP70 + SERPINA12 + LowLevel |
| 2851 | CAD ~ Intercept.High + corin + HSP70 + SERPINA12 + LowLevel |
| 2852 | CAD ~ Intercept.High + corin + HSP70 + SERPINA12 + LowLevel |
| 2853 | CAD ~ Intercept.High + corin + HSP70 + SERPINA12 + LowLevel |
| 2854 | CAD ~ Intercept.High + corin + HSP70 + SERPINA12 + LowLevel |
| 2855 | CAD ~ Intercept.High + corin + HSP70 + SERPINA12 + LowLevel |
| 2856 | CAD ~ Intercept.High + corin + HSP70 + SERPINA12 + LowLevel |
| 2857 | CAD ~ Intercept.High + corin + HSP70 + SERPINA12 + LowLevel |
| 2858 | CAD ~ Intercept.High + corin + HSP70 + LowLevel |
| 2859 | CAD ~ Intercept.High + corin + HSP70 + LowLevel |
| 2860 | CAD ~ Intercept.High + corin + HSP70 + LowLevel |
| 2861 | CAD ~ Intercept.High + corin + HSP70 + LowLevel |
| 2862 | CAD ~ Intercept.High + corin + HSP70 + LowLevel |
| 2863 | CAD ~ Intercept.High + corin + HSP70 + LowLevel |
| 2864 | CAD ~ Intercept.High + corin + HSP70 + LowLevel |
| 2865 | CAD ~ Intercept.High + corin + HSP70 + LowLevel |
| 2866 | CAD ~ Intercept.High + corin + HSP70 + LowLevel |
| 2867 | CAD ~ Intercept.High + corin + HSP70 + LowLevel |
| 2868 | CAD ~ Intercept.High + corin + RBP4 + SERPINA12 + LowLevel |
| 2869 | CAD ~ Intercept.High + corin + RBP4 + SERPINA12 + LowLevel |
| 2870 | CAD ~ Intercept.High + corin + RBP4 + SERPINA12 + LowLevel |
| 2871 | CAD ~ Intercept.High + corin + RBP4 + SERPINA12 + LowLevel |
| 2872 | CAD ~ Intercept.High + corin + RBP4 + SERPINA12 + LowLevel |
| 2873 | CAD ~ Intercept.High + corin + RBP4 + SERPINA12 + LowLevel |
| 2874 | CAD ~ Intercept.High + corin + RBP4 + SERPINA12 + LowLevel |
| 2875 | CAD ~ Intercept.High + corin + RBP4 + SERPINA12 + LowLevel |
| 2876 | CAD ~ Intercept.High + corin + RBP4 + SERPINA12 + LowLevel |
| 2877 | CAD ~ Intercept.High + corin + RBP4 + SERPINA12 + LowLevel |
| 2878 | CAD ~ Intercept.High + corin + RBP4 + LowLevel |
| 2879 | CAD ~ Intercept.High + corin + RBP4 + LowLevel |
| 2880 | CAD ~ Intercept.High + corin + RBP4 + LowLevel |
| 2881 | CAD ~ Intercept.High + corin + RBP4 + LowLevel |
| 2882 | CAD ~ Intercept.High + corin + RBP4 + LowLevel |
| 2883 | CAD ~ Intercept.High + corin + RBP4 + LowLevel |
| 2884 | CAD ~ Intercept.High + corin + RBP4 + LowLevel |
| 2885 | CAD ~ Intercept.High + corin + RBP4 + LowLevel |
| 2886 | CAD ~ Intercept.High + corin + RBP4 + LowLevel |
| 2887 | CAD ~ Intercept.High + corin + RBP4 + LowLevel |
| 2888 | CAD ~ Intercept.High + corin + SERPINA12 + LowLevel |
| 2889 | CAD ~ Intercept.High + corin + SERPINA12 + LowLevel |
| 2890 | CAD ~ Intercept.High + corin + SERPINA12 + LowLevel |
| 2891 | CAD ~ Intercept.High + corin + SERPINA12 + LowLevel |
| 2892 | CAD ~ Intercept.High + corin + SERPINA12 + LowLevel |
| 2893 | CAD ~ Intercept.High + corin + SERPINA12 + LowLevel |
| 2894 | CAD ~ Intercept.High + corin + SERPINA12 + LowLevel |
| 2895 | CAD ~ Intercept.High + corin + SERPINA12 + LowLevel |
| 2896 | CAD ~ Intercept.High + corin + SERPINA12 + LowLevel |
| 2897 | CAD ~ Intercept.High + corin + SERPINA12 + LowLevel |
| 2898 | CAD ~ Intercept.High + corin + LowLevel |
| 2899 | CAD ~ Intercept.High + corin + LowLevel |
| 2900 | CAD ~ Intercept.High + corin + LowLevel |
| 2901 | CAD ~ Intercept.High + corin + LowLevel |
| 2902 | CAD ~ Intercept.High + corin + LowLevel |
| 2903 | CAD ~ Intercept.High + Sex + Age + APOB + corin + LowLevel |
| 2904 | CAD ~ Intercept.High + Sex + Age + APOB + corin + LowLevel |
| 2905 | CAD ~ Intercept.High + Sex + Age + APOB + corin + LowLevel |
| 2906 | CAD ~ Intercept.High + Sex + Age + APOB + corin + LowLevel |
| 2907 | CAD ~ Intercept.High + Sex + Age + APOB + corin + LowLevel |
| 2908 | CAD ~ Intercept.High + Sex + Age + APOB + HSP70 + LowLevel |
| 2909 | CAD ~ Intercept.High + Sex + Age + APOB + HSP70 + LowLevel |
| 2910 | CAD ~ Intercept.High + Sex + Age + APOB + HSP70 + LowLevel |
| 2911 | CAD ~ Intercept.High + Sex + Age + APOB + HSP70 + LowLevel |
| 2912 | CAD ~ Intercept.High + Sex + Age + APOB + HSP70 + LowLevel |
| 2913 | CAD ~ Intercept.High + Sex + Age + APOB + RBP4 + LowLevel |
| 2914 | CAD ~ Intercept.High + Sex + Age + APOB + RBP4 + LowLevel |
| 2915 | CAD ~ Intercept.High + Sex + Age + APOB + RBP4 + LowLevel |
| 2916 | CAD ~ Intercept.High + Sex + Age + APOB + RBP4 + LowLevel |
| 2917 | CAD ~ Intercept.High + Sex + Age + APOB + RBP4 + LowLevel |
| 2918 | CAD ~ Intercept.High + Sex + Age + APOB + SERPINA12 + LowLevel |
| 2919 | CAD ~ Intercept.High + Sex + Age + APOB + SERPINA12 + LowLevel |
| 2920 | CAD ~ Intercept.High + Sex + Age + APOB + SERPINA12 + LowLevel |
| 2921 | CAD ~ Intercept.High + Sex + Age + APOB + SERPINA12 + LowLevel |
| 2922 | CAD ~ Intercept.High + Sex + Age + APOB + SERPINA12 + LowLevel |
| 2923 | CAD ~ Intercept.High + Sex + Age + APOB + LowLevel |
| 2924 | CAD ~ Intercept.High + Sex + Age + APOB + LowLevel |
| 2925 | CAD ~ Intercept.High + Sex + Age + APOB + LowLevel |
| 2926 | CAD ~ Intercept.High + Sex + Age + APOB + LowLevel |
| 2927 | CAD ~ Intercept.High + Sex + Age + APOB + LowLevel |
| 2928 | CAD ~ Intercept.High + Sex + Age + APOB + LowLevel |
| 2929 | CAD ~ Intercept.High + Sex + Age + APOB + LowLevel |
| 2930 | CAD ~ Intercept.High + Sex + Age + APOB + LowLevel |
| 2931 | CAD ~ Intercept.High + Sex + Age + APOB + LowLevel |
| 2932 | CAD ~ Intercept.High + Sex + Age + APOB + LowLevel |
| 2933 | CAD ~ Intercept.High + Sex + Age + corin + HSP70 + LowLevel |
| 2934 | CAD ~ Intercept.High + Sex + Age + corin + HSP70 + LowLevel |
| 2935 | CAD ~ Intercept.High + Sex + Age + corin + HSP70 + LowLevel |
| 2936 | CAD ~ Intercept.High + Sex + Age + corin + HSP70 + LowLevel |
| 2937 | CAD ~ Intercept.High + Sex + Age + corin + HSP70 + LowLevel |
| 2938 | CAD ~ Intercept.High + Sex + Age + corin + RBP4 + LowLevel |
| 2939 | CAD ~ Intercept.High + Sex + Age + corin + RBP4 + LowLevel |
| 2940 | CAD ~ Intercept.High + Sex + Age + corin + RBP4 + LowLevel |
| 2941 | CAD ~ Intercept.High + Sex + Age + corin + RBP4 + LowLevel |
| 2942 | CAD ~ Intercept.High + Sex + Age + corin + RBP4 + LowLevel |
| 2943 | CAD ~ Intercept.High + Sex + Age + corin + SERPINA12 + LowLevel |
| 2944 | CAD ~ Intercept.High + Sex + Age + corin + SERPINA12 + LowLevel |
| 2945 | CAD ~ Intercept.High + Sex + Age + corin + SERPINA12 + LowLevel |
| 2946 | CAD ~ Intercept.High + Sex + Age + corin + SERPINA12 + LowLevel |
| 2947 | CAD ~ Intercept.High + Sex + Age + corin + SERPINA12 + LowLevel |
| 2948 | CAD ~ Intercept.High + Sex + Age + corin + LowLevel |
| 2949 | CAD ~ Intercept.High + Sex + Age + corin + LowLevel |
| 2950 | CAD ~ Intercept.High + Sex + Age + corin + LowLevel |
| 2951 | CAD ~ Intercept.High + Sex + Age + corin + LowLevel |
| 2952 | CAD ~ Intercept.High + Sex + Age + corin + LowLevel |
| 2953 | CAD ~ Intercept.High + Sex + Age + corin + LowLevel |
| 2954 | CAD ~ Intercept.High + Sex + Age + corin + LowLevel |
| 2955 | CAD ~ Intercept.High + Sex + Age + corin + LowLevel |
| 2956 | CAD ~ Intercept.High + Sex + Age + corin + LowLevel |
| 2957 | CAD ~ Intercept.High + Sex + Age + corin + LowLevel |
| 2958 | CAD ~ Intercept.High + Sex + Age + HSP70 + RBP4 + LowLevel |
| 2959 | CAD ~ Intercept.High + Sex + Age + HSP70 + RBP4 + LowLevel |
| 2960 | CAD ~ Intercept.High + Sex + Age + HSP70 + RBP4 + LowLevel |
| 2961 | CAD ~ Intercept.High + Sex + Age + HSP70 + RBP4 + LowLevel |
| 2962 | CAD ~ Intercept.High + Sex + Age + HSP70 + RBP4 + LowLevel |
| 2963 | CAD ~ Intercept.High + Sex + Age + HSP70 + SERPINA12 + LowLevel |
| 2964 | CAD ~ Intercept.High + Sex + Age + HSP70 + SERPINA12 + LowLevel |
| 2965 | CAD ~ Intercept.High + Sex + Age + HSP70 + SERPINA12 + LowLevel |
| 2966 | CAD ~ Intercept.High + Sex + Age + HSP70 + SERPINA12 + LowLevel |
| 2967 | CAD ~ Intercept.High + Sex + Age + HSP70 + SERPINA12 + LowLevel |
| 2968 | CAD ~ Intercept.High + Sex + Age + HSP70 + LowLevel |
| 2969 | CAD ~ Intercept.High + Sex + Age + HSP70 + LowLevel |
| 2970 | CAD ~ Intercept.High + Sex + Age + HSP70 + LowLevel |
| 2971 | CAD ~ Intercept.High + Sex + Age + HSP70 + LowLevel |
| 2972 | CAD ~ Intercept.High + Sex + Age + HSP70 + LowLevel |
| 2973 | CAD ~ Intercept.High + Sex + Age + HSP70 + LowLevel |
| 2974 | CAD ~ Intercept.High + Sex + Age + HSP70 + LowLevel |
| 2975 | CAD ~ Intercept.High + Sex + Age + HSP70 + LowLevel |
| 2976 | CAD ~ Intercept.High + Sex + Age + HSP70 + LowLevel |
| 2977 | CAD ~ Intercept.High + Sex + Age + HSP70 + LowLevel |
| 2978 | CAD ~ Intercept.High + Sex + Age + RBP4 + SERPINA12 + LowLevel |
| 2979 | CAD ~ Intercept.High + Sex + Age + RBP4 + SERPINA12 + LowLevel |
| 2980 | CAD ~ Intercept.High + Sex + Age + RBP4 + SERPINA12 + LowLevel |
| 2981 | CAD ~ Intercept.High + Sex + Age + RBP4 + SERPINA12 + LowLevel |
| 2982 | CAD ~ Intercept.High + Sex + Age + RBP4 + SERPINA12 + LowLevel |
| 2983 | CAD ~ Intercept.High + Sex + Age + RBP4 + LowLevel |
| 2984 | CAD ~ Intercept.High + Sex + Age + RBP4 + LowLevel |
| 2985 | CAD ~ Intercept.High + Sex + Age + RBP4 + LowLevel |
| 2986 | CAD ~ Intercept.High + Sex + Age + RBP4 + LowLevel |
| 2987 | CAD ~ Intercept.High + Sex + Age + RBP4 + LowLevel |
| 2988 | CAD ~ Intercept.High + Sex + Age + RBP4 + LowLevel |
| 2989 | CAD ~ Intercept.High + Sex + Age + RBP4 + LowLevel |
| 2990 | CAD ~ Intercept.High + Sex + Age + RBP4 + LowLevel |
| 2991 | CAD ~ Intercept.High + Sex + Age + RBP4 + LowLevel |
| 2992 | CAD ~ Intercept.High + Sex + Age + RBP4 + LowLevel |
| 2993 | CAD ~ Intercept.High + Sex + Age + SERPINA12 + LowLevel |
| 2994 | CAD ~ Intercept.High + Sex + Age + SERPINA12 + LowLevel |
| 2995 | CAD ~ Intercept.High + Sex + Age + SERPINA12 + LowLevel |
| 2996 | CAD ~ Intercept.High + Sex + Age + SERPINA12 + LowLevel |
| 2997 | CAD ~ Intercept.High + Sex + Age + SERPINA12 + LowLevel |
| 2998 | CAD ~ Intercept.High + Sex + Age + SERPINA12 + LowLevel |
| 2999 | CAD ~ Intercept.High + Sex + Age + SERPINA12 + LowLevel |
| 3000 | CAD ~ Intercept.High + Sex + Age + SERPINA12 + LowLevel |
| 3001 | CAD ~ Intercept.High + Sex + Age + SERPINA12 + LowLevel |
| 3002 | CAD ~ Intercept.High + Sex + Age + SERPINA12 + LowLevel |
| 3003 | CAD ~ Intercept.High + Sex + Age + LowLevel |
| 3004 | CAD ~ Intercept.High + Sex + Age + LowLevel |
| 3005 | CAD ~ Intercept.High + Sex + Age + LowLevel |
| 3006 | CAD ~ Intercept.High + Sex + Age + LowLevel |
| 3007 | CAD ~ Intercept.High + Sex + Age + LowLevel |
| 3008 | CAD ~ Intercept.High + Sex + Age + LowLevel |
| 3009 | CAD ~ Intercept.High + Sex + Age + LowLevel |
| 3010 | CAD ~ Intercept.High + Sex + Age + LowLevel |
| 3011 | CAD ~ Intercept.High + Sex + Age + LowLevel |
| 3012 | CAD ~ Intercept.High + Sex + Age + LowLevel |
| 3013 | CAD ~ Intercept.High + Sex + APOB + corin + HSP70 + LowLevel |
| 3014 | CAD ~ Intercept.High + Sex + APOB + corin + HSP70 + LowLevel |
| 3015 | CAD ~ Intercept.High + Sex + APOB + corin + HSP70 + LowLevel |
| 3016 | CAD ~ Intercept.High + Sex + APOB + corin + HSP70 + LowLevel |
| 3017 | CAD ~ Intercept.High + Sex + APOB + corin + HSP70 + LowLevel |
| 3018 | CAD ~ Intercept.High + Sex + APOB + corin + RBP4 + LowLevel |
| 3019 | CAD ~ Intercept.High + Sex + APOB + corin + RBP4 + LowLevel |
| 3020 | CAD ~ Intercept.High + Sex + APOB + corin + RBP4 + LowLevel |
| 3021 | CAD ~ Intercept.High + Sex + APOB + corin + RBP4 + LowLevel |
| 3022 | CAD ~ Intercept.High + Sex + APOB + corin + RBP4 + LowLevel |
| 3023 | CAD ~ Intercept.High + Sex + APOB + corin + SERPINA12 + LowLevel |
| 3024 | CAD ~ Intercept.High + Sex + APOB + corin + SERPINA12 + LowLevel |
| 3025 | CAD ~ Intercept.High + Sex + APOB + corin + SERPINA12 + LowLevel |
| 3026 | CAD ~ Intercept.High + Sex + APOB + corin + SERPINA12 + LowLevel |
| 3027 | CAD ~ Intercept.High + Sex + APOB + corin + SERPINA12 + LowLevel |
| 3028 | CAD ~ Intercept.High + Sex + APOB + corin + LowLevel |
| 3029 | CAD ~ Intercept.High + Sex + APOB + corin + LowLevel |
| 3030 | CAD ~ Intercept.High + Sex + APOB + corin + LowLevel |
| 3031 | CAD ~ Intercept.High + Sex + APOB + corin + LowLevel |
| 3032 | CAD ~ Intercept.High + Sex + APOB + corin + LowLevel |
| 3033 | CAD ~ Intercept.High + Sex + APOB + corin + LowLevel |
| 3034 | CAD ~ Intercept.High + Sex + APOB + corin + LowLevel |
| 3035 | CAD ~ Intercept.High + Sex + APOB + corin + LowLevel |
| 3036 | CAD ~ Intercept.High + Sex + APOB + corin + LowLevel |
| 3037 | CAD ~ Intercept.High + Sex + APOB + corin + LowLevel |
| 3038 | CAD ~ Intercept.High + Sex + APOB + HSP70 + RBP4 + LowLevel |
| 3039 | CAD ~ Intercept.High + Sex + APOB + HSP70 + RBP4 + LowLevel |
| 3040 | CAD ~ Intercept.High + Sex + APOB + HSP70 + RBP4 + LowLevel |
| 3041 | CAD ~ Intercept.High + Sex + APOB + HSP70 + RBP4 + LowLevel |
| 3042 | CAD ~ Intercept.High + Sex + APOB + HSP70 + RBP4 + LowLevel |
| 3043 | CAD ~ Intercept.High + Sex + APOB + HSP70 + SERPINA12 + LowLevel |
| 3044 | CAD ~ Intercept.High + Sex + APOB + HSP70 + SERPINA12 + LowLevel |
| 3045 | CAD ~ Intercept.High + Sex + APOB + HSP70 + SERPINA12 + LowLevel |
| 3046 | CAD ~ Intercept.High + Sex + APOB + HSP70 + SERPINA12 + LowLevel |
| 3047 | CAD ~ Intercept.High + Sex + APOB + HSP70 + SERPINA12 + LowLevel |
| 3048 | CAD ~ Intercept.High + Sex + APOB + HSP70 + LowLevel |
| 3049 | CAD ~ Intercept.High + Sex + APOB + HSP70 + LowLevel |
| 3050 | CAD ~ Intercept.High + Sex + APOB + HSP70 + LowLevel |
| 3051 | CAD ~ Intercept.High + Sex + APOB + HSP70 + LowLevel |
| 3052 | CAD ~ Intercept.High + Sex + APOB + HSP70 + LowLevel |
| 3053 | CAD ~ Intercept.High + Sex + APOB + HSP70 + LowLevel |
| 3054 | CAD ~ Intercept.High + Sex + APOB + HSP70 + LowLevel |
| 3055 | CAD ~ Intercept.High + Sex + APOB + HSP70 + LowLevel |
| 3056 | CAD ~ Intercept.High + Sex + APOB + HSP70 + LowLevel |
| 3057 | CAD ~ Intercept.High + Sex + APOB + HSP70 + LowLevel |
| 3058 | CAD ~ Intercept.High + Sex + APOB + RBP4 + SERPINA12 + LowLevel |
| 3059 | CAD ~ Intercept.High + Sex + APOB + RBP4 + SERPINA12 + LowLevel |
| 3060 | CAD ~ Intercept.High + Sex + APOB + RBP4 + SERPINA12 + LowLevel |
| 3061 | CAD ~ Intercept.High + Sex + APOB + RBP4 + SERPINA12 + LowLevel |
| 3062 | CAD ~ Intercept.High + Sex + APOB + RBP4 + SERPINA12 + LowLevel |
| 3063 | CAD ~ Intercept.High + Sex + APOB + RBP4 + LowLevel |
| 3064 | CAD ~ Intercept.High + Sex + APOB + RBP4 + LowLevel |
| 3065 | CAD ~ Intercept.High + Sex + APOB + RBP4 + LowLevel |
| 3066 | CAD ~ Intercept.High + Sex + APOB + RBP4 + LowLevel |
| 3067 | CAD ~ Intercept.High + Sex + APOB + RBP4 + LowLevel |
| 3068 | CAD ~ Intercept.High + Sex + APOB + RBP4 + LowLevel |
| 3069 | CAD ~ Intercept.High + Sex + APOB + RBP4 + LowLevel |
| 3070 | CAD ~ Intercept.High + Sex + APOB + RBP4 + LowLevel |
| 3071 | CAD ~ Intercept.High + Sex + APOB + RBP4 + LowLevel |
| 3072 | CAD ~ Intercept.High + Sex + APOB + RBP4 + LowLevel |
| 3073 | CAD ~ Intercept.High + Sex + APOB + SERPINA12 + LowLevel |
| 3074 | CAD ~ Intercept.High + Sex + APOB + SERPINA12 + LowLevel |
| 3075 | CAD ~ Intercept.High + Sex + APOB + SERPINA12 + LowLevel |
| 3076 | CAD ~ Intercept.High + Sex + APOB + SERPINA12 + LowLevel |
| 3077 | CAD ~ Intercept.High + Sex + APOB + SERPINA12 + LowLevel |
| 3078 | CAD ~ Intercept.High + Sex + APOB + SERPINA12 + LowLevel |
| 3079 | CAD ~ Intercept.High + Sex + APOB + SERPINA12 + LowLevel |
| 3080 | CAD ~ Intercept.High + Sex + APOB + SERPINA12 + LowLevel |
| 3081 | CAD ~ Intercept.High + Sex + APOB + SERPINA12 + LowLevel |
| 3082 | CAD ~ Intercept.High + Sex + APOB + SERPINA12 + LowLevel |
| 3083 | CAD ~ Intercept.High + Sex + APOB + LowLevel |
| 3084 | CAD ~ Intercept.High + Sex + APOB + LowLevel |
| 3085 | CAD ~ Intercept.High + Sex + APOB + LowLevel |
| 3086 | CAD ~ Intercept.High + Sex + APOB + LowLevel |
| 3087 | CAD ~ Intercept.High + Sex + APOB + LowLevel |
| 3088 | CAD ~ Intercept.High + Sex + APOB + LowLevel |
| 3089 | CAD ~ Intercept.High + Sex + APOB + LowLevel |
| 3090 | CAD ~ Intercept.High + Sex + APOB + LowLevel |
| 3091 | CAD ~ Intercept.High + Sex + APOB + LowLevel |
| 3092 | CAD ~ Intercept.High + Sex + APOB + LowLevel |
| 3093 | CAD ~ Intercept.High + Sex + corin + HSP70 + RBP4 + LowLevel |
| 3094 | CAD ~ Intercept.High + Sex + corin + HSP70 + RBP4 + LowLevel |
| 3095 | CAD ~ Intercept.High + Sex + corin + HSP70 + RBP4 + LowLevel |
| 3096 | CAD ~ Intercept.High + Sex + corin + HSP70 + RBP4 + LowLevel |
| 3097 | CAD ~ Intercept.High + Sex + corin + HSP70 + RBP4 + LowLevel |
| 3098 | CAD ~ Intercept.High + Sex + corin + HSP70 + SERPINA12 + LowLevel |
| 3099 | CAD ~ Intercept.High + Sex + corin + HSP70 + SERPINA12 + LowLevel |
| 3100 | CAD ~ Intercept.High + Sex + corin + HSP70 + SERPINA12 + LowLevel |
| 3101 | CAD ~ Intercept.High + Sex + corin + HSP70 + SERPINA12 + LowLevel |
| 3102 | CAD ~ Intercept.High + Sex + corin + HSP70 + SERPINA12 + LowLevel |
| 3103 | CAD ~ Intercept.High + Sex + corin + HSP70 + LowLevel |
| 3104 | CAD ~ Intercept.High + Sex + corin + HSP70 + LowLevel |
| 3105 | CAD ~ Intercept.High + Sex + corin + HSP70 + LowLevel |
| 3106 | CAD ~ Intercept.High + Sex + corin + HSP70 + LowLevel |
| 3107 | CAD ~ Intercept.High + Sex + corin + HSP70 + LowLevel |
| 3108 | CAD ~ Intercept.High + Sex + corin + HSP70 + LowLevel |
| 3109 | CAD ~ Intercept.High + Sex + corin + HSP70 + LowLevel |
| 3110 | CAD ~ Intercept.High + Sex + corin + HSP70 + LowLevel |
| 3111 | CAD ~ Intercept.High + Sex + corin + HSP70 + LowLevel |
| 3112 | CAD ~ Intercept.High + Sex + corin + HSP70 + LowLevel |
| 3113 | CAD ~ Intercept.High + Sex + corin + RBP4 + SERPINA12 + LowLevel |
| 3114 | CAD ~ Intercept.High + Sex + corin + RBP4 + SERPINA12 + LowLevel |
| 3115 | CAD ~ Intercept.High + Sex + corin + RBP4 + SERPINA12 + LowLevel |
| 3116 | CAD ~ Intercept.High + Sex + corin + RBP4 + SERPINA12 + LowLevel |
| 3117 | CAD ~ Intercept.High + Sex + corin + RBP4 + SERPINA12 + LowLevel |
| 3118 | CAD ~ Intercept.High + Sex + corin + RBP4 + LowLevel |
| 3119 | CAD ~ Intercept.High + Sex + corin + RBP4 + LowLevel |
| 3120 | CAD ~ Intercept.High + Sex + corin + RBP4 + LowLevel |
| 3121 | CAD ~ Intercept.High + Sex + corin + RBP4 + LowLevel |
| 3122 | CAD ~ Intercept.High + Sex + corin + RBP4 + LowLevel |
| 3123 | CAD ~ Intercept.High + Sex + corin + RBP4 + LowLevel |
| 3124 | CAD ~ Intercept.High + Sex + corin + RBP4 + LowLevel |
| 3125 | CAD ~ Intercept.High + Sex + corin + RBP4 + LowLevel |
| 3126 | CAD ~ Intercept.High + Sex + corin + RBP4 + LowLevel |
| 3127 | CAD ~ Intercept.High + Sex + corin + RBP4 + LowLevel |
| 3128 | CAD ~ Intercept.High + Sex + corin + SERPINA12 + LowLevel |
| 3129 | CAD ~ Intercept.High + Sex + corin + SERPINA12 + LowLevel |
| 3130 | CAD ~ Intercept.High + Sex + corin + SERPINA12 + LowLevel |
| 3131 | CAD ~ Intercept.High + Sex + corin + SERPINA12 + LowLevel |
| 3132 | CAD ~ Intercept.High + Sex + corin + SERPINA12 + LowLevel |
| 3133 | CAD ~ Intercept.High + Sex + corin + SERPINA12 + LowLevel |
| 3134 | CAD ~ Intercept.High + Sex + corin + SERPINA12 + LowLevel |
| 3135 | CAD ~ Intercept.High + Sex + corin + SERPINA12 + LowLevel |
| 3136 | CAD ~ Intercept.High + Sex + corin + SERPINA12 + LowLevel |
| 3137 | CAD ~ Intercept.High + Sex + corin + SERPINA12 + LowLevel |
| 3138 | CAD ~ Intercept.High + Sex + corin + LowLevel |
| 3139 | CAD ~ Intercept.High + Sex + corin + LowLevel |
| 3140 | CAD ~ Intercept.High + Sex + corin + LowLevel |
| 3141 | CAD ~ Intercept.High + Sex + corin + LowLevel |
| 3142 | CAD ~ Intercept.High + Sex + corin + LowLevel |
| 3143 | CAD ~ Intercept.High + Sex + corin + LowLevel |
| 3144 | CAD ~ Intercept.High + Sex + corin + LowLevel |
| 3145 | CAD ~ Intercept.High + Sex + corin + LowLevel |
| 3146 | CAD ~ Intercept.High + Sex + corin + LowLevel |
| 3147 | CAD ~ Intercept.High + Sex + corin + LowLevel |
| 3148 | CAD ~ Intercept.High + Sex + HSP70 + RBP4 + SERPINA12 + LowLevel |
| 3149 | CAD ~ Intercept.High + Sex + HSP70 + RBP4 + SERPINA12 + LowLevel |
| 3150 | CAD ~ Intercept.High + Sex + HSP70 + RBP4 + SERPINA12 + LowLevel |
| 3151 | CAD ~ Intercept.High + Sex + HSP70 + RBP4 + SERPINA12 + LowLevel |
| 3152 | CAD ~ Intercept.High + Sex + HSP70 + RBP4 + SERPINA12 + LowLevel |
| 3153 | CAD ~ Intercept.High + Sex + HSP70 + RBP4 + LowLevel |
| 3154 | CAD ~ Intercept.High + Sex + HSP70 + RBP4 + LowLevel |
| 3155 | CAD ~ Intercept.High + Sex + HSP70 + RBP4 + LowLevel |
| 3156 | CAD ~ Intercept.High + Sex + HSP70 + RBP4 + LowLevel |
| 3157 | CAD ~ Intercept.High + Sex + HSP70 + RBP4 + LowLevel |
| 3158 | CAD ~ Intercept.High + Sex + HSP70 + RBP4 + LowLevel |
| 3159 | CAD ~ Intercept.High + Sex + HSP70 + RBP4 + LowLevel |
| 3160 | CAD ~ Intercept.High + Sex + HSP70 + RBP4 + LowLevel |
| 3161 | CAD ~ Intercept.High + Sex + HSP70 + RBP4 + LowLevel |
| 3162 | CAD ~ Intercept.High + Sex + HSP70 + RBP4 + LowLevel |
| 3163 | CAD ~ Intercept.High + Sex + HSP70 + SERPINA12 + LowLevel |
| 3164 | CAD ~ Intercept.High + Sex + HSP70 + SERPINA12 + LowLevel |
| 3165 | CAD ~ Intercept.High + Sex + HSP70 + SERPINA12 + LowLevel |
| 3166 | CAD ~ Intercept.High + Sex + HSP70 + SERPINA12 + LowLevel |
| 3167 | CAD ~ Intercept.High + Sex + HSP70 + SERPINA12 + LowLevel |
| 3168 | CAD ~ Intercept.High + Sex + HSP70 + SERPINA12 + LowLevel |
| 3169 | CAD ~ Intercept.High + Sex + HSP70 + SERPINA12 + LowLevel |
| 3170 | CAD ~ Intercept.High + Sex + HSP70 + SERPINA12 + LowLevel |
| 3171 | CAD ~ Intercept.High + Sex + HSP70 + SERPINA12 + LowLevel |
| 3172 | CAD ~ Intercept.High + Sex + HSP70 + SERPINA12 + LowLevel |
| 3173 | CAD ~ Intercept.High + Sex + HSP70 + LowLevel |
| 3174 | CAD ~ Intercept.High + Sex + HSP70 + LowLevel |
| 3175 | CAD ~ Intercept.High + Sex + HSP70 + LowLevel |
| 3176 | CAD ~ Intercept.High + Sex + HSP70 + LowLevel |
| 3177 | CAD ~ Intercept.High + Sex + HSP70 + LowLevel |
| 3178 | CAD ~ Intercept.High + Sex + HSP70 + LowLevel |
| 3179 | CAD ~ Intercept.High + Sex + HSP70 + LowLevel |
| 3180 | CAD ~ Intercept.High + Sex + HSP70 + LowLevel |
| 3181 | CAD ~ Intercept.High + Sex + HSP70 + LowLevel |
| 3182 | CAD ~ Intercept.High + Sex + HSP70 + LowLevel |
| 3183 | CAD ~ Intercept.High + Sex + RBP4 + SERPINA12 + LowLevel |
| 3184 | CAD ~ Intercept.High + Sex + RBP4 + SERPINA12 + LowLevel |
| 3185 | CAD ~ Intercept.High + Sex + RBP4 + SERPINA12 + LowLevel |
| 3186 | CAD ~ Intercept.High + Sex + RBP4 + SERPINA12 + LowLevel |
| 3187 | CAD ~ Intercept.High + Sex + RBP4 + SERPINA12 + LowLevel |
| 3188 | CAD ~ Intercept.High + Sex + RBP4 + SERPINA12 + LowLevel |
| 3189 | CAD ~ Intercept.High + Sex + RBP4 + SERPINA12 + LowLevel |
| 3190 | CAD ~ Intercept.High + Sex + RBP4 + SERPINA12 + LowLevel |
| 3191 | CAD ~ Intercept.High + Sex + RBP4 + SERPINA12 + LowLevel |
| 3192 | CAD ~ Intercept.High + Sex + RBP4 + SERPINA12 + LowLevel |
| 3193 | CAD ~ Intercept.High + Sex + RBP4 + LowLevel |
| 3194 | CAD ~ Intercept.High + Sex + RBP4 + LowLevel |
| 3195 | CAD ~ Intercept.High + Sex + RBP4 + LowLevel |
| 3196 | CAD ~ Intercept.High + Sex + RBP4 + LowLevel |
| 3197 | CAD ~ Intercept.High + Sex + RBP4 + LowLevel |
| 3198 | CAD ~ Intercept.High + Sex + RBP4 + LowLevel |
| 3199 | CAD ~ Intercept.High + Sex + RBP4 + LowLevel |
| 3200 | CAD ~ Intercept.High + Sex + RBP4 + LowLevel |
| 3201 | CAD ~ Intercept.High + Sex + RBP4 + LowLevel |
| 3202 | CAD ~ Intercept.High + Sex + RBP4 + LowLevel |
| 3203 | CAD ~ Intercept.High + Sex + SERPINA12 + LowLevel |
| 3204 | CAD ~ Intercept.High + Sex + SERPINA12 + LowLevel |
| 3205 | CAD ~ Intercept.High + Sex + SERPINA12 + LowLevel |
| 3206 | CAD ~ Intercept.High + Sex + SERPINA12 + LowLevel |
| 3207 | CAD ~ Intercept.High + Sex + SERPINA12 + LowLevel |
| 3208 | CAD ~ Intercept.High + Sex + SERPINA12 + LowLevel |
| 3209 | CAD ~ Intercept.High + Sex + SERPINA12 + LowLevel |
| 3210 | CAD ~ Intercept.High + Sex + SERPINA12 + LowLevel |
| 3211 | CAD ~ Intercept.High + Sex + SERPINA12 + LowLevel |
| 3212 | CAD ~ Intercept.High + Sex + SERPINA12 + LowLevel |
| 3213 | CAD ~ Intercept.High + Sex + LowLevel |
| 3214 | CAD ~ Intercept.High + Sex + LowLevel |
| 3215 | CAD ~ Intercept.High + Sex + LowLevel |
| 3216 | CAD ~ Intercept.High + Sex + LowLevel |
| 3217 | CAD ~ Intercept.High + Sex + LowLevel |
| 3218 | CAD ~ Intercept.High + HSP70 + RBP4 + SERPINA12 + LowLevel |
| 3219 | CAD ~ Intercept.High + HSP70 + RBP4 + SERPINA12 + LowLevel |
| 3220 | CAD ~ Intercept.High + HSP70 + RBP4 + SERPINA12 + LowLevel |
| 3221 | CAD ~ Intercept.High + HSP70 + RBP4 + SERPINA12 + LowLevel |
| 3222 | CAD ~ Intercept.High + HSP70 + RBP4 + SERPINA12 + LowLevel |
| 3223 | CAD ~ Intercept.High + HSP70 + RBP4 + SERPINA12 + LowLevel |
| 3224 | CAD ~ Intercept.High + HSP70 + RBP4 + SERPINA12 + LowLevel |
| 3225 | CAD ~ Intercept.High + HSP70 + RBP4 + SERPINA12 + LowLevel |
| 3226 | CAD ~ Intercept.High + HSP70 + RBP4 + SERPINA12 + LowLevel |
| 3227 | CAD ~ Intercept.High + HSP70 + RBP4 + SERPINA12 + LowLevel |
| 3228 | CAD ~ Intercept.High + HSP70 + RBP4 + LowLevel |
| 3229 | CAD ~ Intercept.High + HSP70 + RBP4 + LowLevel |
| 3230 | CAD ~ Intercept.High + HSP70 + RBP4 + LowLevel |
| 3231 | CAD ~ Intercept.High + HSP70 + RBP4 + LowLevel |
| 3232 | CAD ~ Intercept.High + HSP70 + RBP4 + LowLevel |
| 3233 | CAD ~ Intercept.High + HSP70 + RBP4 + LowLevel |
| 3234 | CAD ~ Intercept.High + HSP70 + RBP4 + LowLevel |
| 3235 | CAD ~ Intercept.High + HSP70 + RBP4 + LowLevel |
| 3236 | CAD ~ Intercept.High + HSP70 + RBP4 + LowLevel |
| 3237 | CAD ~ Intercept.High + HSP70 + RBP4 + LowLevel |
| 3238 | CAD ~ Intercept.High + HSP70 + SERPINA12 + LowLevel |
| 3239 | CAD ~ Intercept.High + HSP70 + SERPINA12 + LowLevel |
| 3240 | CAD ~ Intercept.High + HSP70 + SERPINA12 + LowLevel |
| 3241 | CAD ~ Intercept.High + HSP70 + SERPINA12 + LowLevel |
| 3242 | CAD ~ Intercept.High + HSP70 + SERPINA12 + LowLevel |
| 3243 | CAD ~ Intercept.High + HSP70 + SERPINA12 + LowLevel |
| 3244 | CAD ~ Intercept.High + HSP70 + SERPINA12 + LowLevel |
| 3245 | CAD ~ Intercept.High + HSP70 + SERPINA12 + LowLevel |
| 3246 | CAD ~ Intercept.High + HSP70 + SERPINA12 + LowLevel |
| 3247 | CAD ~ Intercept.High + HSP70 + SERPINA12 + LowLevel |
| 3248 | CAD ~ Intercept.High + HSP70 + LowLevel |
| 3249 | CAD ~ Intercept.High + HSP70 + LowLevel |
| 3250 | CAD ~ Intercept.High + HSP70 + LowLevel |
| 3251 | CAD ~ Intercept.High + HSP70 + LowLevel |
| 3252 | CAD ~ Intercept.High + HSP70 + LowLevel |
| 3253 | CAD ~ Intercept.High + RBP4 + SERPINA12 + LowLevel |
| 3254 | CAD ~ Intercept.High + RBP4 + SERPINA12 + LowLevel |
| 3255 | CAD ~ Intercept.High + RBP4 + SERPINA12 + LowLevel |
| 3256 | CAD ~ Intercept.High + RBP4 + SERPINA12 + LowLevel |
| 3257 | CAD ~ Intercept.High + RBP4 + SERPINA12 + LowLevel |
| 3258 | CAD ~ Intercept.High + RBP4 + SERPINA12 + LowLevel |
| 3259 | CAD ~ Intercept.High + RBP4 + SERPINA12 + LowLevel |
| 3260 | CAD ~ Intercept.High + RBP4 + SERPINA12 + LowLevel |
| 3261 | CAD ~ Intercept.High + RBP4 + SERPINA12 + LowLevel |
| 3262 | CAD ~ Intercept.High + RBP4 + SERPINA12 + LowLevel |
| 3263 | CAD ~ Intercept.High + RBP4 + LowLevel |
| 3264 | CAD ~ Intercept.High + RBP4 + LowLevel |
| 3265 | CAD ~ Intercept.High + RBP4 + LowLevel |
| 3266 | CAD ~ Intercept.High + RBP4 + LowLevel |
| 3267 | CAD ~ Intercept.High + RBP4 + LowLevel |
| 3268 | CAD ~ Intercept.High + SERPINA12 + LowLevel |
| 3269 | CAD ~ Intercept.High + SERPINA12 + LowLevel |
| 3270 | CAD ~ Intercept.High + SERPINA12 + LowLevel |
| 3271 | CAD ~ Intercept.High + SERPINA12 + LowLevel |
| 3272 | CAD ~ Intercept.High + SERPINA12 + LowLevel |
| 3273 | CAD ~ Intercept.High + Sex + Age + APOB + corin + HSP70 + RBP4 + SERPINA12 |
| 3274 | CAD ~ Intercept.High + LowLevel |
| 3275 | CAD ~ Intercept.High + Age + APOB + corin + LowLevel |
| 3276 | CAD ~ Intercept.High + Age + APOB + corin + LowLevel |
| 3277 | CAD ~ Intercept.High + Age + APOB + corin + LowLevel |
| 3278 | CAD ~ Intercept.High + Age + APOB + corin + LowLevel |
| 3279 | CAD ~ Intercept.High + Age + APOB + corin + LowLevel |
| 3280 | CAD ~ Intercept.High + Age + APOB + HSP70 + LowLevel |
| 3281 | CAD ~ Intercept.High + Age + APOB + HSP70 + LowLevel |
| 3282 | CAD ~ Intercept.High + Age + APOB + HSP70 + LowLevel |
| 3283 | CAD ~ Intercept.High + Age + APOB + HSP70 + LowLevel |
| 3284 | CAD ~ Intercept.High + Age + APOB + HSP70 + LowLevel |
| 3285 | CAD ~ Intercept.High + Age + APOB + RBP4 + LowLevel |
| 3286 | CAD ~ Intercept.High + Age + APOB + RBP4 + LowLevel |
| 3287 | CAD ~ Intercept.High + Age + APOB + RBP4 + LowLevel |
| 3288 | CAD ~ Intercept.High + Age + APOB + RBP4 + LowLevel |
| 3289 | CAD ~ Intercept.High + Age + APOB + RBP4 + LowLevel |
| 3290 | CAD ~ Intercept.High + Age + APOB + SERPINA12 + LowLevel |
| 3291 | CAD ~ Intercept.High + Age + APOB + SERPINA12 + LowLevel |
| 3292 | CAD ~ Intercept.High + Age + APOB + SERPINA12 + LowLevel |
| 3293 | CAD ~ Intercept.High + Age + APOB + SERPINA12 + LowLevel |
| 3294 | CAD ~ Intercept.High + Age + APOB + SERPINA12 + LowLevel |
| 3295 | CAD ~ Intercept.High + Age + APOB + LowLevel |
| 3296 | CAD ~ Intercept.High + Age + APOB + LowLevel |
| 3297 | CAD ~ Intercept.High + Age + APOB + LowLevel |
| 3298 | CAD ~ Intercept.High + Age + APOB + LowLevel |
| 3299 | CAD ~ Intercept.High + Age + APOB + LowLevel |
| 3300 | CAD ~ Intercept.High + Age + APOB + LowLevel |
| 3301 | CAD ~ Intercept.High + Age + APOB + LowLevel |
| 3302 | CAD ~ Intercept.High + Age + APOB + LowLevel |
| 3303 | CAD ~ Intercept.High + Age + APOB + LowLevel |
| 3304 | CAD ~ Intercept.High + Age + APOB + LowLevel |
| 3305 | CAD ~ Intercept.High + Age + corin + HSP70 + LowLevel |
| 3306 | CAD ~ Intercept.High + Age + corin + HSP70 + LowLevel |
| 3307 | CAD ~ Intercept.High + Age + corin + HSP70 + LowLevel |
| 3308 | CAD ~ Intercept.High + Age + corin + HSP70 + LowLevel |
| 3309 | CAD ~ Intercept.High + Age + corin + HSP70 + LowLevel |
| 3310 | CAD ~ Intercept.High + Age + corin + RBP4 + LowLevel |
| 3311 | CAD ~ Intercept.High + Age + corin + RBP4 + LowLevel |
| 3312 | CAD ~ Intercept.High + Age + corin + RBP4 + LowLevel |
| 3313 | CAD ~ Intercept.High + Age + corin + RBP4 + LowLevel |
| 3314 | CAD ~ Intercept.High + Age + corin + RBP4 + LowLevel |
| 3315 | CAD ~ Intercept.High + Age + corin + SERPINA12 + LowLevel |
| 3316 | CAD ~ Intercept.High + Age + corin + SERPINA12 + LowLevel |
| 3317 | CAD ~ Intercept.High + Age + corin + SERPINA12 + LowLevel |
| 3318 | CAD ~ Intercept.High + Age + corin + SERPINA12 + LowLevel |
| 3319 | CAD ~ Intercept.High + Age + corin + SERPINA12 + LowLevel |
| 3320 | CAD ~ Intercept.High + Age + corin + LowLevel |
| 3321 | CAD ~ Intercept.High + Age + corin + LowLevel |
| 3322 | CAD ~ Intercept.High + Age + corin + LowLevel |
| 3323 | CAD ~ Intercept.High + Age + corin + LowLevel |
| 3324 | CAD ~ Intercept.High + Age + corin + LowLevel |
| 3325 | CAD ~ Intercept.High + Age + corin + LowLevel |
| 3326 | CAD ~ Intercept.High + Age + corin + LowLevel |
| 3327 | CAD ~ Intercept.High + Age + corin + LowLevel |
| 3328 | CAD ~ Intercept.High + Age + corin + LowLevel |
| 3329 | CAD ~ Intercept.High + Age + corin + LowLevel |
| 3330 | CAD ~ Intercept.High + Age + HSP70 + RBP4 + LowLevel |
| 3331 | CAD ~ Intercept.High + Age + HSP70 + RBP4 + LowLevel |
| 3332 | CAD ~ Intercept.High + Age + HSP70 + RBP4 + LowLevel |
| 3333 | CAD ~ Intercept.High + Age + HSP70 + RBP4 + LowLevel |
| 3334 | CAD ~ Intercept.High + Age + HSP70 + RBP4 + LowLevel |
| 3335 | CAD ~ Intercept.High + Age + HSP70 + SERPINA12 + LowLevel |
| 3336 | CAD ~ Intercept.High + Age + HSP70 + SERPINA12 + LowLevel |
| 3337 | CAD ~ Intercept.High + Age + HSP70 + SERPINA12 + LowLevel |
| 3338 | CAD ~ Intercept.High + Age + HSP70 + SERPINA12 + LowLevel |
| 3339 | CAD ~ Intercept.High + Age + HSP70 + SERPINA12 + LowLevel |
| 3340 | CAD ~ Intercept.High + Age + HSP70 + LowLevel |
| 3341 | CAD ~ Intercept.High + Age + HSP70 + LowLevel |
| 3342 | CAD ~ Intercept.High + Age + HSP70 + LowLevel |
| 3343 | CAD ~ Intercept.High + Age + HSP70 + LowLevel |
| 3344 | CAD ~ Intercept.High + Age + HSP70 + LowLevel |
| 3345 | CAD ~ Intercept.High + Age + HSP70 + LowLevel |
| 3346 | CAD ~ Intercept.High + Age + HSP70 + LowLevel |
| 3347 | CAD ~ Intercept.High + Age + HSP70 + LowLevel |
| 3348 | CAD ~ Intercept.High + Age + HSP70 + LowLevel |
| 3349 | CAD ~ Intercept.High + Age + HSP70 + LowLevel |
| 3350 | CAD ~ Intercept.High + Age + RBP4 + SERPINA12 + LowLevel |
| 3351 | CAD ~ Intercept.High + Age + RBP4 + SERPINA12 + LowLevel |
| 3352 | CAD ~ Intercept.High + Age + RBP4 + SERPINA12 + LowLevel |
| 3353 | CAD ~ Intercept.High + Age + RBP4 + SERPINA12 + LowLevel |
| 3354 | CAD ~ Intercept.High + Age + RBP4 + SERPINA12 + LowLevel |
| 3355 | CAD ~ Intercept.High + Age + RBP4 + LowLevel |
| 3356 | CAD ~ Intercept.High + Age + RBP4 + LowLevel |
| 3357 | CAD ~ Intercept.High + Age + RBP4 + LowLevel |
| 3358 | CAD ~ Intercept.High + Age + RBP4 + LowLevel |
| 3359 | CAD ~ Intercept.High + Age + RBP4 + LowLevel |
| 3360 | CAD ~ Intercept.High + Age + RBP4 + LowLevel |
| 3361 | CAD ~ Intercept.High + Age + RBP4 + LowLevel |
| 3362 | CAD ~ Intercept.High + Age + RBP4 + LowLevel |
| 3363 | CAD ~ Intercept.High + Age + RBP4 + LowLevel |
| 3364 | CAD ~ Intercept.High + Age + RBP4 + LowLevel |
| 3365 | CAD ~ Intercept.High + Age + SERPINA12 + LowLevel |
| 3366 | CAD ~ Intercept.High + Age + SERPINA12 + LowLevel |
| 3367 | CAD ~ Intercept.High + Age + SERPINA12 + LowLevel |
| 3368 | CAD ~ Intercept.High + Age + SERPINA12 + LowLevel |
| 3369 | CAD ~ Intercept.High + Age + SERPINA12 + LowLevel |
| 3370 | CAD ~ Intercept.High + Age + SERPINA12 + LowLevel |
| 3371 | CAD ~ Intercept.High + Age + SERPINA12 + LowLevel |
| 3372 | CAD ~ Intercept.High + Age + SERPINA12 + LowLevel |
| 3373 | CAD ~ Intercept.High + Age + SERPINA12 + LowLevel |
| 3374 | CAD ~ Intercept.High + Age + SERPINA12 + LowLevel |
| 3375 | CAD ~ Intercept.High + Age + LowLevel |
| 3376 | CAD ~ Intercept.High + Age + LowLevel |
| 3377 | CAD ~ Intercept.High + Age + LowLevel |
| 3378 | CAD ~ Intercept.High + Age + LowLevel |
| 3379 | CAD ~ Intercept.High + Age + LowLevel |
| 3380 | CAD ~ Intercept.High + Age + LowLevel |
| 3381 | CAD ~ Intercept.High + Age + LowLevel |
| 3382 | CAD ~ Intercept.High + Age + LowLevel |
| 3383 | CAD ~ Intercept.High + Age + LowLevel |
| 3384 | CAD ~ Intercept.High + Age + LowLevel |
| 3385 | CAD ~ Intercept.High + APOB + corin + HSP70 + LowLevel |
| 3386 | CAD ~ Intercept.High + APOB + corin + HSP70 + LowLevel |
| 3387 | CAD ~ Intercept.High + APOB + corin + HSP70 + LowLevel |
| 3388 | CAD ~ Intercept.High + APOB + corin + HSP70 + LowLevel |
| 3389 | CAD ~ Intercept.High + APOB + corin + HSP70 + LowLevel |
| 3390 | CAD ~ Intercept.High + APOB + corin + RBP4 + LowLevel |
| 3391 | CAD ~ Intercept.High + APOB + corin + RBP4 + LowLevel |
| 3392 | CAD ~ Intercept.High + APOB + corin + RBP4 + LowLevel |
| 3393 | CAD ~ Intercept.High + APOB + corin + RBP4 + LowLevel |
| 3394 | CAD ~ Intercept.High + APOB + corin + RBP4 + LowLevel |
| 3395 | CAD ~ Intercept.High + APOB + corin + SERPINA12 + LowLevel |
| 3396 | CAD ~ Intercept.High + APOB + corin + SERPINA12 + LowLevel |
| 3397 | CAD ~ Intercept.High + APOB + corin + SERPINA12 + LowLevel |
| 3398 | CAD ~ Intercept.High + APOB + corin + SERPINA12 + LowLevel |
| 3399 | CAD ~ Intercept.High + APOB + corin + SERPINA12 + LowLevel |
| 3400 | CAD ~ Intercept.High + APOB + corin + LowLevel |
| 3401 | CAD ~ Intercept.High + APOB + corin + LowLevel |
| 3402 | CAD ~ Intercept.High + APOB + corin + LowLevel |
| 3403 | CAD ~ Intercept.High + APOB + corin + LowLevel |
| 3404 | CAD ~ Intercept.High + APOB + corin + LowLevel |
| 3405 | CAD ~ Intercept.High + APOB + corin + LowLevel |
| 3406 | CAD ~ Intercept.High + APOB + corin + LowLevel |
| 3407 | CAD ~ Intercept.High + APOB + corin + LowLevel |
| 3408 | CAD ~ Intercept.High + APOB + corin + LowLevel |
| 3409 | CAD ~ Intercept.High + APOB + corin + LowLevel |
| 3410 | CAD ~ Intercept.High + APOB + HSP70 + RBP4 + LowLevel |
| 3411 | CAD ~ Intercept.High + APOB + HSP70 + RBP4 + LowLevel |
| 3412 | CAD ~ Intercept.High + APOB + HSP70 + RBP4 + LowLevel |
| 3413 | CAD ~ Intercept.High + APOB + HSP70 + RBP4 + LowLevel |
| 3414 | CAD ~ Intercept.High + APOB + HSP70 + RBP4 + LowLevel |
| 3415 | CAD ~ Intercept.High + APOB + HSP70 + SERPINA12 + LowLevel |
| 3416 | CAD ~ Intercept.High + APOB + HSP70 + SERPINA12 + LowLevel |
| 3417 | CAD ~ Intercept.High + APOB + HSP70 + SERPINA12 + LowLevel |
| 3418 | CAD ~ Intercept.High + APOB + HSP70 + SERPINA12 + LowLevel |
| 3419 | CAD ~ Intercept.High + APOB + HSP70 + SERPINA12 + LowLevel |
| 3420 | CAD ~ Intercept.High + APOB + HSP70 + LowLevel |
| 3421 | CAD ~ Intercept.High + APOB + HSP70 + LowLevel |
| 3422 | CAD ~ Intercept.High + APOB + HSP70 + LowLevel |
| 3423 | CAD ~ Intercept.High + APOB + HSP70 + LowLevel |
| 3424 | CAD ~ Intercept.High + APOB + HSP70 + LowLevel |
| 3425 | CAD ~ Intercept.High + APOB + HSP70 + LowLevel |
| 3426 | CAD ~ Intercept.High + APOB + HSP70 + LowLevel |
| 3427 | CAD ~ Intercept.High + APOB + HSP70 + LowLevel |
| 3428 | CAD ~ Intercept.High + APOB + HSP70 + LowLevel |
| 3429 | CAD ~ Intercept.High + APOB + HSP70 + LowLevel |
| 3430 | CAD ~ Intercept.High + APOB + RBP4 + SERPINA12 + LowLevel |
| 3431 | CAD ~ Intercept.High + APOB + RBP4 + SERPINA12 + LowLevel |
| 3432 | CAD ~ Intercept.High + APOB + RBP4 + SERPINA12 + LowLevel |
| 3433 | CAD ~ Intercept.High + APOB + RBP4 + SERPINA12 + LowLevel |
| 3434 | CAD ~ Intercept.High + APOB + RBP4 + SERPINA12 + LowLevel |
| 3435 | CAD ~ Intercept.High + APOB + RBP4 + LowLevel |
| 3436 | CAD ~ Intercept.High + APOB + RBP4 + LowLevel |
| 3437 | CAD ~ Intercept.High + APOB + RBP4 + LowLevel |
| 3438 | CAD ~ Intercept.High + APOB + RBP4 + LowLevel |
| 3439 | CAD ~ Intercept.High + APOB + RBP4 + LowLevel |
| 3440 | CAD ~ Intercept.High + APOB + RBP4 + LowLevel |
| 3441 | CAD ~ Intercept.High + APOB + RBP4 + LowLevel |
| 3442 | CAD ~ Intercept.High + APOB + RBP4 + LowLevel |
| 3443 | CAD ~ Intercept.High + APOB + RBP4 + LowLevel |
| 3444 | CAD ~ Intercept.High + APOB + RBP4 + LowLevel |
| 3445 | CAD ~ Intercept.High + APOB + SERPINA12 + LowLevel |
| 3446 | CAD ~ Intercept.High + APOB + SERPINA12 + LowLevel |
| 3447 | CAD ~ Intercept.High + APOB + SERPINA12 + LowLevel |
| 3448 | CAD ~ Intercept.High + APOB + SERPINA12 + LowLevel |
| 3449 | CAD ~ Intercept.High + APOB + SERPINA12 + LowLevel |
| 3450 | CAD ~ Intercept.High + APOB + SERPINA12 + LowLevel |
| 3451 | CAD ~ Intercept.High + APOB + SERPINA12 + LowLevel |
| 3452 | CAD ~ Intercept.High + APOB + SERPINA12 + LowLevel |
| 3453 | CAD ~ Intercept.High + APOB + SERPINA12 + LowLevel |
| 3454 | CAD ~ Intercept.High + APOB + SERPINA12 + LowLevel |
| 3455 | CAD ~ Intercept.High + APOB + LowLevel |
| 3456 | CAD ~ Intercept.High + APOB + LowLevel |
| 3457 | CAD ~ Intercept.High + APOB + LowLevel |
| 3458 | CAD ~ Intercept.High + APOB + LowLevel |
| 3459 | CAD ~ Intercept.High + APOB + LowLevel |
| 3460 | CAD ~ Intercept.High + APOB + LowLevel |
| 3461 | CAD ~ Intercept.High + APOB + LowLevel |
| 3462 | CAD ~ Intercept.High + APOB + LowLevel |
| 3463 | CAD ~ Intercept.High + APOB + LowLevel |
| 3464 | CAD ~ Intercept.High + APOB + LowLevel |
| 3465 | CAD ~ Intercept.High + corin + HSP70 + RBP4 + LowLevel |
| 3466 | CAD ~ Intercept.High + corin + HSP70 + RBP4 + LowLevel |
| 3467 | CAD ~ Intercept.High + corin + HSP70 + RBP4 + LowLevel |
| 3468 | CAD ~ Intercept.High + corin + HSP70 + RBP4 + LowLevel |
| 3469 | CAD ~ Intercept.High + corin + HSP70 + RBP4 + LowLevel |
| 3470 | CAD ~ Intercept.High + corin + HSP70 + SERPINA12 + LowLevel |
| 3471 | CAD ~ Intercept.High + corin + HSP70 + SERPINA12 + LowLevel |
| 3472 | CAD ~ Intercept.High + corin + HSP70 + SERPINA12 + LowLevel |
| 3473 | CAD ~ Intercept.High + corin + HSP70 + SERPINA12 + LowLevel |
| 3474 | CAD ~ Intercept.High + corin + HSP70 + SERPINA12 + LowLevel |
| 3475 | CAD ~ Intercept.High + corin + HSP70 + LowLevel |
| 3476 | CAD ~ Intercept.High + corin + HSP70 + LowLevel |
| 3477 | CAD ~ Intercept.High + corin + HSP70 + LowLevel |
| 3478 | CAD ~ Intercept.High + corin + HSP70 + LowLevel |
| 3479 | CAD ~ Intercept.High + corin + HSP70 + LowLevel |
| 3480 | CAD ~ Intercept.High + corin + HSP70 + LowLevel |
| 3481 | CAD ~ Intercept.High + corin + HSP70 + LowLevel |
| 3482 | CAD ~ Intercept.High + corin + HSP70 + LowLevel |
| 3483 | CAD ~ Intercept.High + corin + HSP70 + LowLevel |
| 3484 | CAD ~ Intercept.High + corin + HSP70 + LowLevel |
| 3485 | CAD ~ Intercept.High + corin + RBP4 + SERPINA12 + LowLevel |
| 3486 | CAD ~ Intercept.High + corin + RBP4 + SERPINA12 + LowLevel |
| 3487 | CAD ~ Intercept.High + corin + RBP4 + SERPINA12 + LowLevel |
| 3488 | CAD ~ Intercept.High + corin + RBP4 + SERPINA12 + LowLevel |
| 3489 | CAD ~ Intercept.High + corin + RBP4 + SERPINA12 + LowLevel |
| 3490 | CAD ~ Intercept.High + corin + RBP4 + LowLevel |
| 3491 | CAD ~ Intercept.High + corin + RBP4 + LowLevel |
| 3492 | CAD ~ Intercept.High + corin + RBP4 + LowLevel |
| 3493 | CAD ~ Intercept.High + corin + RBP4 + LowLevel |
| 3494 | CAD ~ Intercept.High + corin + RBP4 + LowLevel |
| 3495 | CAD ~ Intercept.High + corin + RBP4 + LowLevel |
| 3496 | CAD ~ Intercept.High + corin + RBP4 + LowLevel |
| 3497 | CAD ~ Intercept.High + corin + RBP4 + LowLevel |
| 3498 | CAD ~ Intercept.High + corin + RBP4 + LowLevel |
| 3499 | CAD ~ Intercept.High + corin + RBP4 + LowLevel |
| 3500 | CAD ~ Intercept.High + corin + SERPINA12 + LowLevel |
| 3501 | CAD ~ Intercept.High + corin + SERPINA12 + LowLevel |
| 3502 | CAD ~ Intercept.High + corin + SERPINA12 + LowLevel |
| 3503 | CAD ~ Intercept.High + corin + SERPINA12 + LowLevel |
| 3504 | CAD ~ Intercept.High + corin + SERPINA12 + LowLevel |
| 3505 | CAD ~ Intercept.High + corin + SERPINA12 + LowLevel |
| 3506 | CAD ~ Intercept.High + corin + SERPINA12 + LowLevel |
| 3507 | CAD ~ Intercept.High + corin + SERPINA12 + LowLevel |
| 3508 | CAD ~ Intercept.High + corin + SERPINA12 + LowLevel |
| 3509 | CAD ~ Intercept.High + corin + SERPINA12 + LowLevel |
| 3510 | CAD ~ Intercept.High + corin + LowLevel |
| 3511 | CAD ~ Intercept.High + corin + LowLevel |
| 3512 | CAD ~ Intercept.High + corin + LowLevel |
| 3513 | CAD ~ Intercept.High + corin + LowLevel |
| 3514 | CAD ~ Intercept.High + corin + LowLevel |
| 3515 | CAD ~ Intercept.High + corin + LowLevel |
| 3516 | CAD ~ Intercept.High + corin + LowLevel |
| 3517 | CAD ~ Intercept.High + corin + LowLevel |
| 3518 | CAD ~ Intercept.High + corin + LowLevel |
| 3519 | CAD ~ Intercept.High + corin + LowLevel |
| 3520 | CAD ~ Intercept.High + Sex + Age + APOB + LowLevel |
| 3521 | CAD ~ Intercept.High + Sex + Age + APOB + LowLevel |
| 3522 | CAD ~ Intercept.High + Sex + Age + APOB + LowLevel |
| 3523 | CAD ~ Intercept.High + Sex + Age + APOB + LowLevel |
| 3524 | CAD ~ Intercept.High + Sex + Age + APOB + LowLevel |
| 3525 | CAD ~ Intercept.High + Sex + Age + corin + LowLevel |
| 3526 | CAD ~ Intercept.High + Sex + Age + corin + LowLevel |
| 3527 | CAD ~ Intercept.High + Sex + Age + corin + LowLevel |
| 3528 | CAD ~ Intercept.High + Sex + Age + corin + LowLevel |
| 3529 | CAD ~ Intercept.High + Sex + Age + corin + LowLevel |
| 3530 | CAD ~ Intercept.High + Sex + Age + HSP70 + LowLevel |
| 3531 | CAD ~ Intercept.High + Sex + Age + HSP70 + LowLevel |
| 3532 | CAD ~ Intercept.High + Sex + Age + HSP70 + LowLevel |
| 3533 | CAD ~ Intercept.High + Sex + Age + HSP70 + LowLevel |
| 3534 | CAD ~ Intercept.High + Sex + Age + HSP70 + LowLevel |
| 3535 | CAD ~ Intercept.High + Sex + Age + RBP4 + LowLevel |
| 3536 | CAD ~ Intercept.High + Sex + Age + RBP4 + LowLevel |
| 3537 | CAD ~ Intercept.High + Sex + Age + RBP4 + LowLevel |
| 3538 | CAD ~ Intercept.High + Sex + Age + RBP4 + LowLevel |
| 3539 | CAD ~ Intercept.High + Sex + Age + RBP4 + LowLevel |
| 3540 | CAD ~ Intercept.High + Sex + Age + SERPINA12 + LowLevel |
| 3541 | CAD ~ Intercept.High + Sex + Age + SERPINA12 + LowLevel |
| 3542 | CAD ~ Intercept.High + Sex + Age + SERPINA12 + LowLevel |
| 3543 | CAD ~ Intercept.High + Sex + Age + SERPINA12 + LowLevel |
| 3544 | CAD ~ Intercept.High + Sex + Age + SERPINA12 + LowLevel |
| 3545 | CAD ~ Intercept.High + Sex + Age + LowLevel |
| 3546 | CAD ~ Intercept.High + Sex + Age + LowLevel |
| 3547 | CAD ~ Intercept.High + Sex + Age + LowLevel |
| 3548 | CAD ~ Intercept.High + Sex + Age + LowLevel |
| 3549 | CAD ~ Intercept.High + Sex + Age + LowLevel |
| 3550 | CAD ~ Intercept.High + Sex + Age + LowLevel |
| 3551 | CAD ~ Intercept.High + Sex + Age + LowLevel |
| 3552 | CAD ~ Intercept.High + Sex + Age + LowLevel |
| 3553 | CAD ~ Intercept.High + Sex + Age + LowLevel |
| 3554 | CAD ~ Intercept.High + Sex + Age + LowLevel |
| 3555 | CAD ~ Intercept.High + Sex + APOB + corin + LowLevel |
| 3556 | CAD ~ Intercept.High + Sex + APOB + corin + LowLevel |
| 3557 | CAD ~ Intercept.High + Sex + APOB + corin + LowLevel |
| 3558 | CAD ~ Intercept.High + Sex + APOB + corin + LowLevel |
| 3559 | CAD ~ Intercept.High + Sex + APOB + corin + LowLevel |
| 3560 | CAD ~ Intercept.High + Sex + APOB + HSP70 + LowLevel |
| 3561 | CAD ~ Intercept.High + Sex + APOB + HSP70 + LowLevel |
| 3562 | CAD ~ Intercept.High + Sex + APOB + HSP70 + LowLevel |
| 3563 | CAD ~ Intercept.High + Sex + APOB + HSP70 + LowLevel |
| 3564 | CAD ~ Intercept.High + Sex + APOB + HSP70 + LowLevel |
| 3565 | CAD ~ Intercept.High + Sex + APOB + RBP4 + LowLevel |
| 3566 | CAD ~ Intercept.High + Sex + APOB + RBP4 + LowLevel |
| 3567 | CAD ~ Intercept.High + Sex + APOB + RBP4 + LowLevel |
| 3568 | CAD ~ Intercept.High + Sex + APOB + RBP4 + LowLevel |
| 3569 | CAD ~ Intercept.High + Sex + APOB + RBP4 + LowLevel |
| 3570 | CAD ~ Intercept.High + Sex + APOB + SERPINA12 + LowLevel |
| 3571 | CAD ~ Intercept.High + Sex + APOB + SERPINA12 + LowLevel |
| 3572 | CAD ~ Intercept.High + Sex + APOB + SERPINA12 + LowLevel |
| 3573 | CAD ~ Intercept.High + Sex + APOB + SERPINA12 + LowLevel |
| 3574 | CAD ~ Intercept.High + Sex + APOB + SERPINA12 + LowLevel |
| 3575 | CAD ~ Intercept.High + Sex + APOB + LowLevel |
| 3576 | CAD ~ Intercept.High + Sex + APOB + LowLevel |
| 3577 | CAD ~ Intercept.High + Sex + APOB + LowLevel |
| 3578 | CAD ~ Intercept.High + Sex + APOB + LowLevel |
| 3579 | CAD ~ Intercept.High + Sex + APOB + LowLevel |
| 3580 | CAD ~ Intercept.High + Sex + APOB + LowLevel |
| 3581 | CAD ~ Intercept.High + Sex + APOB + LowLevel |
| 3582 | CAD ~ Intercept.High + Sex + APOB + LowLevel |
| 3583 | CAD ~ Intercept.High + Sex + APOB + LowLevel |
| 3584 | CAD ~ Intercept.High + Sex + APOB + LowLevel |
| 3585 | CAD ~ Intercept.High + Sex + corin + HSP70 + LowLevel |
| 3586 | CAD ~ Intercept.High + Sex + corin + HSP70 + LowLevel |
| 3587 | CAD ~ Intercept.High + Sex + corin + HSP70 + LowLevel |
| 3588 | CAD ~ Intercept.High + Sex + corin + HSP70 + LowLevel |
| 3589 | CAD ~ Intercept.High + Sex + corin + HSP70 + LowLevel |
| 3590 | CAD ~ Intercept.High + Sex + corin + RBP4 + LowLevel |
| 3591 | CAD ~ Intercept.High + Sex + corin + RBP4 + LowLevel |
| 3592 | CAD ~ Intercept.High + Sex + corin + RBP4 + LowLevel |
| 3593 | CAD ~ Intercept.High + Sex + corin + RBP4 + LowLevel |
| 3594 | CAD ~ Intercept.High + Sex + corin + RBP4 + LowLevel |
| 3595 | CAD ~ Intercept.High + Sex + corin + SERPINA12 + LowLevel |
| 3596 | CAD ~ Intercept.High + Sex + corin + SERPINA12 + LowLevel |
| 3597 | CAD ~ Intercept.High + Sex + corin + SERPINA12 + LowLevel |
| 3598 | CAD ~ Intercept.High + Sex + corin + SERPINA12 + LowLevel |
| 3599 | CAD ~ Intercept.High + Sex + corin + SERPINA12 + LowLevel |
| 3600 | CAD ~ Intercept.High + Sex + corin + LowLevel |
| 3601 | CAD ~ Intercept.High + Sex + corin + LowLevel |
| 3602 | CAD ~ Intercept.High + Sex + corin + LowLevel |
| 3603 | CAD ~ Intercept.High + Sex + corin + LowLevel |
| 3604 | CAD ~ Intercept.High + Sex + corin + LowLevel |
| 3605 | CAD ~ Intercept.High + Sex + corin + LowLevel |
| 3606 | CAD ~ Intercept.High + Sex + corin + LowLevel |
| 3607 | CAD ~ Intercept.High + Sex + corin + LowLevel |
| 3608 | CAD ~ Intercept.High + Sex + corin + LowLevel |
| 3609 | CAD ~ Intercept.High + Sex + corin + LowLevel |
| 3610 | CAD ~ Intercept.High + Sex + HSP70 + RBP4 + LowLevel |
| 3611 | CAD ~ Intercept.High + Sex + HSP70 + RBP4 + LowLevel |
| 3612 | CAD ~ Intercept.High + Sex + HSP70 + RBP4 + LowLevel |
| 3613 | CAD ~ Intercept.High + Sex + HSP70 + RBP4 + LowLevel |
| 3614 | CAD ~ Intercept.High + Sex + HSP70 + RBP4 + LowLevel |
| 3615 | CAD ~ Intercept.High + Sex + HSP70 + SERPINA12 + LowLevel |
| 3616 | CAD ~ Intercept.High + Sex + HSP70 + SERPINA12 + LowLevel |
| 3617 | CAD ~ Intercept.High + Sex + HSP70 + SERPINA12 + LowLevel |
| 3618 | CAD ~ Intercept.High + Sex + HSP70 + SERPINA12 + LowLevel |
| 3619 | CAD ~ Intercept.High + Sex + HSP70 + SERPINA12 + LowLevel |
| 3620 | CAD ~ Intercept.High + Sex + HSP70 + LowLevel |
| 3621 | CAD ~ Intercept.High + Sex + HSP70 + LowLevel |
| 3622 | CAD ~ Intercept.High + Sex + HSP70 + LowLevel |
| 3623 | CAD ~ Intercept.High + Sex + HSP70 + LowLevel |
| 3624 | CAD ~ Intercept.High + Sex + HSP70 + LowLevel |
| 3625 | CAD ~ Intercept.High + Sex + HSP70 + LowLevel |
| 3626 | CAD ~ Intercept.High + Sex + HSP70 + LowLevel |
| 3627 | CAD ~ Intercept.High + Sex + HSP70 + LowLevel |
| 3628 | CAD ~ Intercept.High + Sex + HSP70 + LowLevel |
| 3629 | CAD ~ Intercept.High + Sex + HSP70 + LowLevel |
| 3630 | CAD ~ Intercept.High + Sex + RBP4 + SERPINA12 + LowLevel |
| 3631 | CAD ~ Intercept.High + Sex + RBP4 + SERPINA12 + LowLevel |
| 3632 | CAD ~ Intercept.High + Sex + RBP4 + SERPINA12 + LowLevel |
| 3633 | CAD ~ Intercept.High + Sex + RBP4 + SERPINA12 + LowLevel |
| 3634 | CAD ~ Intercept.High + Sex + RBP4 + SERPINA12 + LowLevel |
| 3635 | CAD ~ Intercept.High + Sex + RBP4 + LowLevel |
| 3636 | CAD ~ Intercept.High + Sex + RBP4 + LowLevel |
| 3637 | CAD ~ Intercept.High + Sex + RBP4 + LowLevel |
| 3638 | CAD ~ Intercept.High + Sex + RBP4 + LowLevel |
| 3639 | CAD ~ Intercept.High + Sex + RBP4 + LowLevel |
| 3640 | CAD ~ Intercept.High + Sex + RBP4 + LowLevel |
| 3641 | CAD ~ Intercept.High + Sex + RBP4 + LowLevel |
| 3642 | CAD ~ Intercept.High + Sex + RBP4 + LowLevel |
| 3643 | CAD ~ Intercept.High + Sex + RBP4 + LowLevel |
| 3644 | CAD ~ Intercept.High + Sex + RBP4 + LowLevel |
| 3645 | CAD ~ Intercept.High + Sex + SERPINA12 + LowLevel |
| 3646 | CAD ~ Intercept.High + Sex + SERPINA12 + LowLevel |
| 3647 | CAD ~ Intercept.High + Sex + SERPINA12 + LowLevel |
| 3648 | CAD ~ Intercept.High + Sex + SERPINA12 + LowLevel |
| 3649 | CAD ~ Intercept.High + Sex + SERPINA12 + LowLevel |
| 3650 | CAD ~ Intercept.High + Sex + SERPINA12 + LowLevel |
| 3651 | CAD ~ Intercept.High + Sex + SERPINA12 + LowLevel |
| 3652 | CAD ~ Intercept.High + Sex + SERPINA12 + LowLevel |
| 3653 | CAD ~ Intercept.High + Sex + SERPINA12 + LowLevel |
| 3654 | CAD ~ Intercept.High + Sex + SERPINA12 + LowLevel |
| 3655 | CAD ~ Intercept.High + Sex + LowLevel |
| 3656 | CAD ~ Intercept.High + Sex + LowLevel |
| 3657 | CAD ~ Intercept.High + Sex + LowLevel |
| 3658 | CAD ~ Intercept.High + Sex + LowLevel |
| 3659 | CAD ~ Intercept.High + Sex + LowLevel |
| 3660 | CAD ~ Intercept.High + Sex + LowLevel |
| 3661 | CAD ~ Intercept.High + Sex + LowLevel |
| 3662 | CAD ~ Intercept.High + Sex + LowLevel |
| 3663 | CAD ~ Intercept.High + Sex + LowLevel |
| 3664 | CAD ~ Intercept.High + Sex + LowLevel |
| 3665 | CAD ~ Intercept.High + HSP70 + RBP4 + SERPINA12 + LowLevel |
| 3666 | CAD ~ Intercept.High + HSP70 + RBP4 + SERPINA12 + LowLevel |
| 3667 | CAD ~ Intercept.High + HSP70 + RBP4 + SERPINA12 + LowLevel |
| 3668 | CAD ~ Intercept.High + HSP70 + RBP4 + SERPINA12 + LowLevel |
| 3669 | CAD ~ Intercept.High + HSP70 + RBP4 + SERPINA12 + LowLevel |
| 3670 | CAD ~ Intercept.High + HSP70 + RBP4 + LowLevel |
| 3671 | CAD ~ Intercept.High + HSP70 + RBP4 + LowLevel |
| 3672 | CAD ~ Intercept.High + HSP70 + RBP4 + LowLevel |
| 3673 | CAD ~ Intercept.High + HSP70 + RBP4 + LowLevel |
| 3674 | CAD ~ Intercept.High + HSP70 + RBP4 + LowLevel |
| 3675 | CAD ~ Intercept.High + HSP70 + RBP4 + LowLevel |
| 3676 | CAD ~ Intercept.High + HSP70 + RBP4 + LowLevel |
| 3677 | CAD ~ Intercept.High + HSP70 + RBP4 + LowLevel |
| 3678 | CAD ~ Intercept.High + HSP70 + RBP4 + LowLevel |
| 3679 | CAD ~ Intercept.High + HSP70 + RBP4 + LowLevel |
| 3680 | CAD ~ Intercept.High + HSP70 + SERPINA12 + LowLevel |
| 3681 | CAD ~ Intercept.High + HSP70 + SERPINA12 + LowLevel |
| 3682 | CAD ~ Intercept.High + HSP70 + SERPINA12 + LowLevel |
| 3683 | CAD ~ Intercept.High + HSP70 + SERPINA12 + LowLevel |
| 3684 | CAD ~ Intercept.High + HSP70 + SERPINA12 + LowLevel |
| 3685 | CAD ~ Intercept.High + HSP70 + SERPINA12 + LowLevel |
| 3686 | CAD ~ Intercept.High + HSP70 + SERPINA12 + LowLevel |
| 3687 | CAD ~ Intercept.High + HSP70 + SERPINA12 + LowLevel |
| 3688 | CAD ~ Intercept.High + HSP70 + SERPINA12 + LowLevel |
| 3689 | CAD ~ Intercept.High + HSP70 + SERPINA12 + LowLevel |
| 3690 | CAD ~ Intercept.High + HSP70 + LowLevel |
| 3691 | CAD ~ Intercept.High + HSP70 + LowLevel |
| 3692 | CAD ~ Intercept.High + HSP70 + LowLevel |
| 3693 | CAD ~ Intercept.High + HSP70 + LowLevel |
| 3694 | CAD ~ Intercept.High + HSP70 + LowLevel |
| 3695 | CAD ~ Intercept.High + HSP70 + LowLevel |
| 3696 | CAD ~ Intercept.High + HSP70 + LowLevel |
| 3697 | CAD ~ Intercept.High + HSP70 + LowLevel |
| 3698 | CAD ~ Intercept.High + HSP70 + LowLevel |
| 3699 | CAD ~ Intercept.High + HSP70 + LowLevel |
| 3700 | CAD ~ Intercept.High + RBP4 + SERPINA12 + LowLevel |
| 3701 | CAD ~ Intercept.High + RBP4 + SERPINA12 + LowLevel |
| 3702 | CAD ~ Intercept.High + RBP4 + SERPINA12 + LowLevel |
| 3703 | CAD ~ Intercept.High + RBP4 + SERPINA12 + LowLevel |
| 3704 | CAD ~ Intercept.High + RBP4 + SERPINA12 + LowLevel |
| 3705 | CAD ~ Intercept.High + RBP4 + SERPINA12 + LowLevel |
| 3706 | CAD ~ Intercept.High + RBP4 + SERPINA12 + LowLevel |
| 3707 | CAD ~ Intercept.High + RBP4 + SERPINA12 + LowLevel |
| 3708 | CAD ~ Intercept.High + RBP4 + SERPINA12 + LowLevel |
| 3709 | CAD ~ Intercept.High + RBP4 + SERPINA12 + LowLevel |
| 3710 | CAD ~ Intercept.High + RBP4 + LowLevel |
| 3711 | CAD ~ Intercept.High + RBP4 + LowLevel |
| 3712 | CAD ~ Intercept.High + RBP4 + LowLevel |
| 3713 | CAD ~ Intercept.High + RBP4 + LowLevel |
| 3714 | CAD ~ Intercept.High + RBP4 + LowLevel |
| 3715 | CAD ~ Intercept.High + RBP4 + LowLevel |
| 3716 | CAD ~ Intercept.High + RBP4 + LowLevel |
| 3717 | CAD ~ Intercept.High + RBP4 + LowLevel |
| 3718 | CAD ~ Intercept.High + RBP4 + LowLevel |
| 3719 | CAD ~ Intercept.High + RBP4 + LowLevel |
| 3720 | CAD ~ Intercept.High + SERPINA12 + LowLevel |
| 3721 | CAD ~ Intercept.High + SERPINA12 + LowLevel |
| 3722 | CAD ~ Intercept.High + SERPINA12 + LowLevel |
| 3723 | CAD ~ Intercept.High + SERPINA12 + LowLevel |
| 3724 | CAD ~ Intercept.High + SERPINA12 + LowLevel |
| 3725 | CAD ~ Intercept.High + SERPINA12 + LowLevel |
| 3726 | CAD ~ Intercept.High + SERPINA12 + LowLevel |
| 3727 | CAD ~ Intercept.High + SERPINA12 + LowLevel |
| 3728 | CAD ~ Intercept.High + SERPINA12 + LowLevel |
| 3729 | CAD ~ Intercept.High + SERPINA12 + LowLevel |
| 3730 | CAD ~ Intercept.High + Age + APOB + corin + HSP70 + RBP4 + SERPINA12 |
| 3731 | CAD ~ Intercept.High + Sex + Age + APOB + corin + HSP70 + RBP4 |
| 3732 | CAD ~ Intercept.High + Sex + Age + APOB + corin + HSP70 + SERPINA12 |
| 3733 | CAD ~ Intercept.High + Sex + Age + APOB + corin + RBP4 + SERPINA12 |
| 3734 | CAD ~ Intercept.High + Sex + Age + APOB + HSP70 + RBP4 + SERPINA12 |
| 3735 | CAD ~ Intercept.High + Sex + Age + corin + HSP70 + RBP4 + SERPINA12 |
| 3736 | CAD ~ Intercept.High + Sex + APOB + corin + HSP70 + RBP4 + SERPINA12 |
| 3737 | CAD ~ Intercept.High + LowLevel |
| 3738 | CAD ~ Intercept.High + LowLevel |
| 3739 | CAD ~ Intercept.High + LowLevel |
| 3740 | CAD ~ Intercept.High + LowLevel |
| 3741 | CAD ~ Intercept.High + LowLevel |
| 3742 | CAD ~ Intercept.High + Age + APOB + LowLevel |
| 3743 | CAD ~ Intercept.High + Age + APOB + LowLevel |
| 3744 | CAD ~ Intercept.High + Age + APOB + LowLevel |
| 3745 | CAD ~ Intercept.High + Age + APOB + LowLevel |
| 3746 | CAD ~ Intercept.High + Age + APOB + LowLevel |
| 3747 | CAD ~ Intercept.High + Age + corin + LowLevel |
| 3748 | CAD ~ Intercept.High + Age + corin + LowLevel |
| 3749 | CAD ~ Intercept.High + Age + corin + LowLevel |
| 3750 | CAD ~ Intercept.High + Age + corin + LowLevel |
| 3751 | CAD ~ Intercept.High + Age + corin + LowLevel |
| 3752 | CAD ~ Intercept.High + Age + HSP70 + LowLevel |
| 3753 | CAD ~ Intercept.High + Age + HSP70 + LowLevel |
| 3754 | CAD ~ Intercept.High + Age + HSP70 + LowLevel |
| 3755 | CAD ~ Intercept.High + Age + HSP70 + LowLevel |
| 3756 | CAD ~ Intercept.High + Age + HSP70 + LowLevel |
| 3757 | CAD ~ Intercept.High + Age + RBP4 + LowLevel |
| 3758 | CAD ~ Intercept.High + Age + RBP4 + LowLevel |
| 3759 | CAD ~ Intercept.High + Age + RBP4 + LowLevel |
| 3760 | CAD ~ Intercept.High + Age + RBP4 + LowLevel |
| 3761 | CAD ~ Intercept.High + Age + RBP4 + LowLevel |
| 3762 | CAD ~ Intercept.High + Age + SERPINA12 + LowLevel |
| 3763 | CAD ~ Intercept.High + Age + SERPINA12 + LowLevel |
| 3764 | CAD ~ Intercept.High + Age + SERPINA12 + LowLevel |
| 3765 | CAD ~ Intercept.High + Age + SERPINA12 + LowLevel |
| 3766 | CAD ~ Intercept.High + Age + SERPINA12 + LowLevel |
| 3767 | CAD ~ Intercept.High + Age + LowLevel |
| 3768 | CAD ~ Intercept.High + Age + LowLevel |
| 3769 | CAD ~ Intercept.High + Age + LowLevel |
| 3770 | CAD ~ Intercept.High + Age + LowLevel |
| 3771 | CAD ~ Intercept.High + Age + LowLevel |
| 3772 | CAD ~ Intercept.High + Age + LowLevel |
| 3773 | CAD ~ Intercept.High + Age + LowLevel |
| 3774 | CAD ~ Intercept.High + Age + LowLevel |
| 3775 | CAD ~ Intercept.High + Age + LowLevel |
| 3776 | CAD ~ Intercept.High + Age + LowLevel |
| 3777 | CAD ~ Intercept.High + APOB + corin + LowLevel |
| 3778 | CAD ~ Intercept.High + APOB + corin + LowLevel |
| 3779 | CAD ~ Intercept.High + APOB + corin + LowLevel |
| 3780 | CAD ~ Intercept.High + APOB + corin + LowLevel |
| 3781 | CAD ~ Intercept.High + APOB + corin + LowLevel |
| 3782 | CAD ~ Intercept.High + APOB + HSP70 + LowLevel |
| 3783 | CAD ~ Intercept.High + APOB + HSP70 + LowLevel |
| 3784 | CAD ~ Intercept.High + APOB + HSP70 + LowLevel |
| 3785 | CAD ~ Intercept.High + APOB + HSP70 + LowLevel |
| 3786 | CAD ~ Intercept.High + APOB + HSP70 + LowLevel |
| 3787 | CAD ~ Intercept.High + APOB + RBP4 + LowLevel |
| 3788 | CAD ~ Intercept.High + APOB + RBP4 + LowLevel |
| 3789 | CAD ~ Intercept.High + APOB + RBP4 + LowLevel |
| 3790 | CAD ~ Intercept.High + APOB + RBP4 + LowLevel |
| 3791 | CAD ~ Intercept.High + APOB + RBP4 + LowLevel |
| 3792 | CAD ~ Intercept.High + APOB + SERPINA12 + LowLevel |
| 3793 | CAD ~ Intercept.High + APOB + SERPINA12 + LowLevel |
| 3794 | CAD ~ Intercept.High + APOB + SERPINA12 + LowLevel |
| 3795 | CAD ~ Intercept.High + APOB + SERPINA12 + LowLevel |
| 3796 | CAD ~ Intercept.High + APOB + SERPINA12 + LowLevel |
| 3797 | CAD ~ Intercept.High + APOB + LowLevel |
| 3798 | CAD ~ Intercept.High + APOB + LowLevel |
| 3799 | CAD ~ Intercept.High + APOB + LowLevel |
| 3800 | CAD ~ Intercept.High + APOB + LowLevel |
| 3801 | CAD ~ Intercept.High + APOB + LowLevel |
| 3802 | CAD ~ Intercept.High + APOB + LowLevel |
| 3803 | CAD ~ Intercept.High + APOB + LowLevel |
| 3804 | CAD ~ Intercept.High + APOB + LowLevel |
| 3805 | CAD ~ Intercept.High + APOB + LowLevel |
| 3806 | CAD ~ Intercept.High + APOB + LowLevel |
| 3807 | CAD ~ Intercept.High + corin + HSP70 + LowLevel |
| 3808 | CAD ~ Intercept.High + corin + HSP70 + LowLevel |
| 3809 | CAD ~ Intercept.High + corin + HSP70 + LowLevel |
| 3810 | CAD ~ Intercept.High + corin + HSP70 + LowLevel |
| 3811 | CAD ~ Intercept.High + corin + HSP70 + LowLevel |
| 3812 | CAD ~ Intercept.High + corin + RBP4 + LowLevel |
| 3813 | CAD ~ Intercept.High + corin + RBP4 + LowLevel |
| 3814 | CAD ~ Intercept.High + corin + RBP4 + LowLevel |
| 3815 | CAD ~ Intercept.High + corin + RBP4 + LowLevel |
| 3816 | CAD ~ Intercept.High + corin + RBP4 + LowLevel |
| 3817 | CAD ~ Intercept.High + corin + SERPINA12 + LowLevel |
| 3818 | CAD ~ Intercept.High + corin + SERPINA12 + LowLevel |
| 3819 | CAD ~ Intercept.High + corin + SERPINA12 + LowLevel |
| 3820 | CAD ~ Intercept.High + corin + SERPINA12 + LowLevel |
| 3821 | CAD ~ Intercept.High + corin + SERPINA12 + LowLevel |
| 3822 | CAD ~ Intercept.High + corin + LowLevel |
| 3823 | CAD ~ Intercept.High + corin + LowLevel |
| 3824 | CAD ~ Intercept.High + corin + LowLevel |
| 3825 | CAD ~ Intercept.High + corin + LowLevel |
| 3826 | CAD ~ Intercept.High + corin + LowLevel |
| 3827 | CAD ~ Intercept.High + corin + LowLevel |
| 3828 | CAD ~ Intercept.High + corin + LowLevel |
| 3829 | CAD ~ Intercept.High + corin + LowLevel |
| 3830 | CAD ~ Intercept.High + corin + LowLevel |
| 3831 | CAD ~ Intercept.High + corin + LowLevel |
| 3832 | CAD ~ Intercept.High + Sex + Age + LowLevel |
| 3833 | CAD ~ Intercept.High + Sex + Age + LowLevel |
| 3834 | CAD ~ Intercept.High + Sex + Age + LowLevel |
| 3835 | CAD ~ Intercept.High + Sex + Age + LowLevel |
| 3836 | CAD ~ Intercept.High + Sex + Age + LowLevel |
| 3837 | CAD ~ Intercept.High + Sex + APOB + LowLevel |
| 3838 | CAD ~ Intercept.High + Sex + APOB + LowLevel |
| 3839 | CAD ~ Intercept.High + Sex + APOB + LowLevel |
| 3840 | CAD ~ Intercept.High + Sex + APOB + LowLevel |
| 3841 | CAD ~ Intercept.High + Sex + APOB + LowLevel |
| 3842 | CAD ~ Intercept.High + Sex + corin + LowLevel |
| 3843 | CAD ~ Intercept.High + Sex + corin + LowLevel |
| 3844 | CAD ~ Intercept.High + Sex + corin + LowLevel |
| 3845 | CAD ~ Intercept.High + Sex + corin + LowLevel |
| 3846 | CAD ~ Intercept.High + Sex + corin + LowLevel |
| 3847 | CAD ~ Intercept.High + Sex + HSP70 + LowLevel |
| 3848 | CAD ~ Intercept.High + Sex + HSP70 + LowLevel |
| 3849 | CAD ~ Intercept.High + Sex + HSP70 + LowLevel |
| 3850 | CAD ~ Intercept.High + Sex + HSP70 + LowLevel |
| 3851 | CAD ~ Intercept.High + Sex + HSP70 + LowLevel |
| 3852 | CAD ~ Intercept.High + Sex + RBP4 + LowLevel |
| 3853 | CAD ~ Intercept.High + Sex + RBP4 + LowLevel |
| 3854 | CAD ~ Intercept.High + Sex + RBP4 + LowLevel |
| 3855 | CAD ~ Intercept.High + Sex + RBP4 + LowLevel |
| 3856 | CAD ~ Intercept.High + Sex + RBP4 + LowLevel |
| 3857 | CAD ~ Intercept.High + Sex + SERPINA12 + LowLevel |
| 3858 | CAD ~ Intercept.High + Sex + SERPINA12 + LowLevel |
| 3859 | CAD ~ Intercept.High + Sex + SERPINA12 + LowLevel |
| 3860 | CAD ~ Intercept.High + Sex + SERPINA12 + LowLevel |
| 3861 | CAD ~ Intercept.High + Sex + SERPINA12 + LowLevel |
| 3862 | CAD ~ Intercept.High + Sex + LowLevel |
| 3863 | CAD ~ Intercept.High + Sex + LowLevel |
| 3864 | CAD ~ Intercept.High + Sex + LowLevel |
| 3865 | CAD ~ Intercept.High + Sex + LowLevel |
| 3866 | CAD ~ Intercept.High + Sex + LowLevel |
| 3867 | CAD ~ Intercept.High + Sex + LowLevel |
| 3868 | CAD ~ Intercept.High + Sex + LowLevel |
| 3869 | CAD ~ Intercept.High + Sex + LowLevel |
| 3870 | CAD ~ Intercept.High + Sex + LowLevel |
| 3871 | CAD ~ Intercept.High + Sex + LowLevel |
| 3872 | CAD ~ Intercept.High + HSP70 + RBP4 + LowLevel |
| 3873 | CAD ~ Intercept.High + HSP70 + RBP4 + LowLevel |
| 3874 | CAD ~ Intercept.High + HSP70 + RBP4 + LowLevel |
| 3875 | CAD ~ Intercept.High + HSP70 + RBP4 + LowLevel |
| 3876 | CAD ~ Intercept.High + HSP70 + RBP4 + LowLevel |
| 3877 | CAD ~ Intercept.High + HSP70 + SERPINA12 + LowLevel |
| 3878 | CAD ~ Intercept.High + HSP70 + SERPINA12 + LowLevel |
| 3879 | CAD ~ Intercept.High + HSP70 + SERPINA12 + LowLevel |
| 3880 | CAD ~ Intercept.High + HSP70 + SERPINA12 + LowLevel |
| 3881 | CAD ~ Intercept.High + HSP70 + SERPINA12 + LowLevel |
| 3882 | CAD ~ Intercept.High + HSP70 + LowLevel |
| 3883 | CAD ~ Intercept.High + HSP70 + LowLevel |
| 3884 | CAD ~ Intercept.High + HSP70 + LowLevel |
| 3885 | CAD ~ Intercept.High + HSP70 + LowLevel |
| 3886 | CAD ~ Intercept.High + HSP70 + LowLevel |
| 3887 | CAD ~ Intercept.High + HSP70 + LowLevel |
| 3888 | CAD ~ Intercept.High + HSP70 + LowLevel |
| 3889 | CAD ~ Intercept.High + HSP70 + LowLevel |
| 3890 | CAD ~ Intercept.High + HSP70 + LowLevel |
| 3891 | CAD ~ Intercept.High + HSP70 + LowLevel |
| 3892 | CAD ~ Intercept.High + RBP4 + SERPINA12 + LowLevel |
| 3893 | CAD ~ Intercept.High + RBP4 + SERPINA12 + LowLevel |
| 3894 | CAD ~ Intercept.High + RBP4 + SERPINA12 + LowLevel |
| 3895 | CAD ~ Intercept.High + RBP4 + SERPINA12 + LowLevel |
| 3896 | CAD ~ Intercept.High + RBP4 + SERPINA12 + LowLevel |
| 3897 | CAD ~ Intercept.High + RBP4 + LowLevel |
| 3898 | CAD ~ Intercept.High + RBP4 + LowLevel |
| 3899 | CAD ~ Intercept.High + RBP4 + LowLevel |
| 3900 | CAD ~ Intercept.High + RBP4 + LowLevel |
| 3901 | CAD ~ Intercept.High + RBP4 + LowLevel |
| 3902 | CAD ~ Intercept.High + RBP4 + LowLevel |
| 3903 | CAD ~ Intercept.High + RBP4 + LowLevel |
| 3904 | CAD ~ Intercept.High + RBP4 + LowLevel |
| 3905 | CAD ~ Intercept.High + RBP4 + LowLevel |
| 3906 | CAD ~ Intercept.High + RBP4 + LowLevel |
| 3907 | CAD ~ Intercept.High + SERPINA12 + LowLevel |
| 3908 | CAD ~ Intercept.High + SERPINA12 + LowLevel |
| 3909 | CAD ~ Intercept.High + SERPINA12 + LowLevel |
| 3910 | CAD ~ Intercept.High + SERPINA12 + LowLevel |
| 3911 | CAD ~ Intercept.High + SERPINA12 + LowLevel |
| 3912 | CAD ~ Intercept.High + SERPINA12 + LowLevel |
| 3913 | CAD ~ Intercept.High + SERPINA12 + LowLevel |
| 3914 | CAD ~ Intercept.High + SERPINA12 + LowLevel |
| 3915 | CAD ~ Intercept.High + SERPINA12 + LowLevel |
| 3916 | CAD ~ Intercept.High + SERPINA12 + LowLevel |
| 3917 | CAD ~ Intercept.High + Age + APOB + corin + HSP70 + RBP4 |
| 3918 | CAD ~ Intercept.High + Age + APOB + corin + HSP70 + 5ERPINA12 |
| 3919 | CAD ~ Intercept.High + Age + APOB + corin + RBP4 + SERPINA12 |
| 3920 | CAD ~ Intercept.High + Age + APOB + HSP70 + RBP4 + SERPINA12 |
| 3921 | CAD ~ Intercept.High + Age + corin + HSP70 + RBP4 + SERPINA12 |
| 3922 | CAD ~ Intercept.High + APOB + corin + HSP70 + RBP4 + SERPINA12 |
| 3923 | CAD ~ Intercept.High + Sex + Age + APOB + corin + HSP70 |
| 3924 | CAD ~ Intercept.High + Sex + Age + APOB + corin + RBP4 |
| 3925 | CAD ~ Intercept.High + Sex + Age + APOB + corin + SERPINA12 |
| 3926 | CAD ~ Intercept.High + Sex + Age + APOB + HSP70 + RBP4 |
| 3927 | CAD ~ Intercept.High + Sex + Age + APOB + HSP70 + SERPINA12 |
| 3928 | CAD ~ Intercept.High + Sex + Age + APOB + RBP4 + SERPINA12 |
| 3929 | CAD ~ Intercept.High + Sex + Age + corin + HSP70 + RBP4 |
| 3930 | CAD ~ Intercept.High + Sex + Age + corin + HSP70 + SERPINA12 |
| 3931 | CAD ~ Intercept.High + Sex + Age + corin + RBP4 + SERPINA12 |
| 3932 | CAD ~ Intercept.High + Sex + Age + HSP70 + RBP4 + SERPINA12 |
| 3933 | CAD ~ Intercept.High + Sex + APOB + corin + HSP70 + RBP4 |
| 3934 | CAD ~ Intercept.High + Sex + APOB + corin + HSP70 + SERPINA12 |
| 3935 | CAD ~ Intercept.High + Sex + APOB + corin + RBP4 + SERPINA12 |
| 3936 | CAD ~ Intercept.High + Sex + APOB + HSP70 + RBP4 + SERPINA12 |
| 3937 | CAD ~ Intercept.High + Sex + corin + HSP70 + RBP4 + SERPINA12 |
| 3938 | CAD ~ Intercept.High + LowLevel |
| 3939 | CAD ~ Intercept.High + LowLevel |
| 3940 | CAD ~ Intercept.High + LowLevel |
| 3941 | CAD ~ Intercept.High + LowLevel |
| 3942 | CAD ~ Intercept.High + LowLevel |
| 3943 | CAD ~ Intercept.High + LowLevel |
| 3944 | CAD ~ Intercept.High + LowLevel |
| 3945 | CAD ~ Intercept.High + LowLevel |
| 3946 | CAD ~ Intercept.High + LowLevel |
| 3947 | CAD ~ Intercept.High + LowLevel |
| 3948 | CAD ~ Intercept.High + Age + LowLevel |
| 3949 | CAD ~ Intercept.High + Age + LowLevel |
| 3950 | CAD ~ Intercept.High + Age + LowLevel |
| 3951 | CAD ~ Intercept.High + Age + LowLevel |
| 3952 | CAD ~ Intercept.High + Age + LowLevel |
| 3953 | CAD ~ Intercept.High + APOB + LowLevel |
| 3954 | CAD ~ Intercept.High + APOB + LowLevel |
| 3955 | CAD ~ Intercept.High + APOB + LowLevel |
| 3956 | CAD ~ Intercept.High + APOB + LowLevel |
| 3957 | CAD ~ Intercept.High + APOB + LowLevel |
| 3958 | CAD ~ Intercept.High + corin + LowLevel |
| 3959 | CAD ~ Intercept.High + corin + LowLevel |
| 3960 | CAD ~ Intercept.High + corin + LowLevel |
| 3961 | CAD ~ Intercept.High + corin + LowLevel |
| 3962 | CAD ~ Intercept.High + corin + LowLevel |
| 3963 | CAD ~ Intercept.High + Sex + LowLevel |
| 3964 | CAD ~ Intercept.High + Sex + LowLevel |
| 3965 | CAD ~ Intercept.High + Sex + LowLevel |
| 3966 | CAD ~ Intercept.High + Sex + LowLevel |
| 3967 | CAD ~ Intercept.High + Sex + LowLevel |
| 3968 | CAD ~ Intercept.High + HSP70 + LowLevel |
| 3969 | CAD ~ Intercept.High + HSP70 + LowLevel |
| 3970 | CAD ~ Intercept.High + HSP70 + LowLevel |
| 3971 | CAD ~ Intercept.High + HSP70 + LowLevel |
| 3972 | CAD ~ Intercept.High + HSP70 + LowLevel |
| 3973 | CAD ~ Intercept.High + RBP4 + LowLevel |
| 3974 | CAD ~ Intercept.High + RBP4 + LowLevel |
| 3975 | CAD ~ Intercept.High + RBP4 + LowLevel |
| 3976 | CAD ~ Intercept.High + RBP4 + LowLevel |
| 3977 | CAD ~ Intercept.High + RBP4 + LowLevel |
| 3978 | CAD ~ Intercept.High + SERPINA12 + LowLevel |
| 3979 | CAD ~ Intercept.High + SERPINA12 + LowLevel |
| 3980 | CAD ~ Intercept.High + SERPINA12 + LowLevel |
| 3981 | CAD ~ Intercept.High + SERPINA12 + LowLevel |
| 3982 | CAD ~ Intercept.High + SERPINA12 + LowLevel |
| 3983 | CAD ~ Intercept.High + Age + APOB + corin + HSP70 |
| 3984 | CAD ~ Intercept.High + Age + APOB + corin + RBP4 |
| 3985 | CAD ~ Intercept.High + Age + APOB + corin + SERPINA12 |
| 3986 | CAD ~ Intercept.High + Age + APOB + HSP70 + RBP4 |
| 3987 | CAD ~ Intercept.High + Age + APOB + HSP70 + SERPINA12 |
| 3988 | CAD ~ Intercept.High + Age + APOB + RBP4 + SERPINA12 |
| 3989 | CAD ~ Intercept.High + Age + corin + HSP70 + RBP4 |
| 3990 | CAD ~ Intercept.High + Age + corin + HSP70 + SERPINA12 |
| 3991 | CAD ~ Intercept.High + Age + corin + RBP4 + SERPINA12 |
| 3992 | CAD ~ Intercept.High + Age + HSP70 + RBP4 + SERPINA12 |
| 3993 | CAD ~ Intercept.High + APOB + corin + HSP70 + RBP4 |
| 3994 | CAD ~ Intercept.High + APOB + corin + HSP70 + SERPINA12 |
| 3995 | CAD ~ Intercept.High + APOB + corin + RBP4 + SERPINA12 |
| 3996 | CAD ~ Intercept.High + APOB + HSP70 + RBP4 + SERPINA12 |
| 3997 | CAD ~ Intercept.High + corin + HSP70 + RBP4 + SERPINA12 |
| 3998 | CAD ~ Intercept.High + Sex + Age + APOB + corin |
| 3999 | CAD ~ Intercept.High + Sex + Age + APOB + HSP70 |
| 4000 | CAD ~ Intercept.High + Sex + Age + APOB + RBP4 |
| 4001 | CAD ~ Intercept.High + Sex + Age + APOB + SERPINA12 |
| 4002 | CAD ~ Intercept.High + Sex + Age + corin + HSP70 |
| 4003 | CAD ~ Intercept.High + Sex + Age + corin + RBP4 |
| 4004 | CAD ~ Intercept.High + Sex + Age + corin + SERPINA12 |
| 4005 | CAD ~ Intercept.High + Sex + Age + HSP70 + RBP4 |
| 4006 | CAD ~ Intercept.High + Sex + Age + HSP70 + SERPINA12 |
| 4007 | CAD ~ Intercept.High + Sex + Age + RBP4 + SERPINA12 |
| 4008 | CAD ~ Intercept.High + Sex + APOB + corin + HSP70 |
| 4009 | CAD ~ Intercept.High + Sex + APOB + corin + RBP4 |
| 4010 | CAD ~ Intercept.High + Sex + APOB + corin + SERPINA12 |
| 4011 | CAD ~ Intercept.High + Sex + APOB + HSP70 + RBP4 |
| 4012 | CAD ~ Intercept.High + Sex + APOB + HSP70 + SERPINA12 |
| 4013 | CAD ~ Intercept.High + Sex + APOB + RBP4 + SERPINA12 |
| 4014 | CAD ~ Intercept.High + Sex + corin + HSP70 + RBP4 |
| 4015 | CAD ~ Intercept.High + Sex + corin + HSP70 + SERPINA12 |
| 4016 | CAD ~ Intercept.High + Sex + corin + RBP4 + SERPINA12 |
| 4017 | CAD ~ Intercept.High + Sex + HSP70 + RBP4 + SERPINA12 |
| 4018 | CAD ~ Intercept.High + LowLevel |
| 4019 | CAD ~ Intercept.High + LowLevel |
| 4020 | CAD ~ Intercept.High + LowLevel |
| 4021 | CAD ~ Intercept.High + LowLevel |
| 4022 | CAD ~ Intercept.High + LowLevel |
| 4023 | CAD ~ Intercept.High + LowLevel |
| 4024 | CAD ~ Intercept.High + LowLevel |
| 4025 | CAD ~ Intercept.High + LowLevel |
| 4026 | CAD ~ Intercept.High + LowLevel |
| 4027 | CAD ~ Intercept.High + LowLevel |
| 4028 | CAD ~ Intercept.High + Age + APOB + corin |
| 4029 | CAD ~ Intercept.High + Age + APOB + HSP70 |
| 4030 | CAD ~ Intercept.High + Age + APOB + RBP4 |
| 4031 | CAD ~ Intercept.High + Age + APOB + SERPINA12 |
| 4032 | CAD ~ Intercept.High + Age + corin + HSP70 |
| 4033 | CAD ~ Intercept.High + Age + corin + RBP4 |
| 4034 | CAD ~ Intercept.High + Age + corin + SERPINA12 |
| 4035 | CAD ~ Intercept.High + Age + HSP70 + RBP4 |
| 4036 | CAD ~ Intercept.High + Age + HSP70 + SERPINA12 |
| 4037 | CAD ~ Intercept.High + Age + RBP4 + SERPINA12 |
| 4038 | CAD ~ Intercept.High + APOB + corin + HSP70 |
| 4039 | CAD ~ Intercept.High + APOB + corin + RBP4 |
| 4040 | CAD ~ Intercept.High + APOB + corin + SERPINA12 |
| 4041 | CAD ~ Intercept.High + APOB + HSP70 + RBP4 |
| 4042 | CAD ~ Intercept.High + APOB + HSP70 + SERPINA12 |
| 4043 | CAD ~ Intercept.High + APOB + RBP4 + SERPINA12 |
| 4044 | CAD ~ Intercept.High + corin + HSP70 + RBP4 |
| 4045 | CAD ~ Intercept.High + corin + HSP70 + SERPINA12 |
| 4046 | CAD ~ Intercept.High + corin + RBP4 + SERPINA12 |
| 4047 | CAD ~ Intercept.High + Sex + Age + APOB |
| 4048 | CAD ~ Intercept.High + Sex + Age + corin |
| 4049 | CAD ~ Intercept.High + Sex + Age + HSP70 |
| 4050 | CAD ~ Intercept.High + Sex + Age + RBP4 |
| 4051 | CAD ~ Intercept.High + Sex + Age + SERPINA12 |
| 4052 | CAD ~ Intercept.High + Sex + APOB + corin |
| 4053 | CAD ~ Intercept.High + Sex + APOB + HSP70 |
| 4054 | CAD ~ Intercept.High + Sex + APOB + RBP4 |
| 4055 | CAD ~ Intercept.High + Sex + APOB + SERPINA12 |
| 4056 | CAD ~ Intercept.High + Sex + corin + HSP70 |
| 4057 | CAD ~ Intercept.High + Sex + corin + RBP4 |
| 4058 | CAD ~ Intercept.High + Sex + corin + SERPINA12 |
| 4059 | CAD ~ Intercept.High + Sex + HSP70 + RBP4 |
| 4060 | CAD ~ Intercept.High + Sex + HSP70 + SERPINA12 |
| 4061 | CAD ~ Intercept.High + Sex + RBP4 + SERPINA12 |
| 4062 | CAD ~ Intercept.High + HSP70 + RBP4 + SERPINA12 |
| 4063 | CAD ~ Intercept.High + LowLevel |
| 4064 | CAD ~ Intercept.High + LowLevel |
| 4065 | CAD ~ Intercept.High + LowLevel |
| 4066 | CAD ~ Intercept.High + LowLevel |
| 4067 | CAD ~ Intercept.High + LowLevel |
| 4068 | CAD ~ Intercept.High + Age + APOB |
| 4069 | CAD ~ Intercept.High + Age + corin |
| 4070 | CAD ~ Intercept.High + Age + HSP70 |
| 4071 | CAD ~ Intercept.High + Age + RBP4 |
| 4072 | CAD ~ Intercept.High + Age + SERPINA12 |
| 4073 | CAD ~ Intercept.High + APOB + corin |
| 4074 | CAD ~ Intercept.High + APOB + HSP70 |
| 4075 | CAD ~ Intercept.High + APOB + RBP4 |
| 4076 | CAD ~ Intercept.High + APOB + SERPINA12 |
| 4077 | CAD ~ Intercept.High + corin + HSP70 |
| 4078 | CAD ~ Intercept.High + corin + RBP4 |
| 4079 | CAD ~ Intercept.High + corin + SERPINA12 |
| 4080 | CAD ~ Intercept.High + Sex + Age |
| 4081 | CAD ~ Intercept.High + Sex + APOB |
| 4082 | CAD ~ Intercept.High + Sex + corin |
| 4083 | CAD ~ Intercept.High + Sex + HSP70 |
| 4084 | CAD ~ Intercept.High + Sex + RBP4 |
| 4085 | CAD ~ Intercept.High + Sex + SERPINA12 |
| 4086 | CAD ~ Intercept.High + HSP70 + RBP4 |
| 4087 | CAD ~ Intercept.High + HSP70 + SERPINA12 |
| 4088 | CAD ~ Intercept.High + RBP4 + SERPINA12 |
| 4089 | CAD ~ Intercept.High + Age |
| 4090 | CAD ~ Intercept.High + APOB |
| 4091 | CAD ~ Intercept.High + corin |
| 4092 | CAD ~ Intercept.High + Sex |
| 4093 | CAD ~ Intercept.High + HSP70 |
| 4094 | CAD ~ Intercept.High + RBP4 |
| 4095 | CAD ~ Intercept.High + SERPINA12 |

| | LowLevel |
|---|---|
| 1 | CAD ~ Intercept.Low + AdipA1 + NTproBNP + PlGF + A8MPO + A12TNF |
| 2 | CAD ~ Intercept.Low + AdipA1 + NTproBNP + PlGF + A8MPO + A12TNF |
| 3 | CAD ~ Intercept.Low + AdipA1 + NTproBNP + PlGF + A8MPO |
| 4 | CAD ~ Intercept.Low + AdipA1 + NTproBNP + PlGF + A12TNF |
| 5 | CAD ~ Intercept.Low + AdipA1 + NTproBNP + A8MPO + A12TNF |
| 6 | CAD ~ Intercept.Low + AdipA1 + PlGF + A8MPO + A12TNF |
| 7 | CAD ~ Intercept.Low + NTproBNP + PlGF + A8MPO + A12TNF |
| 8 | CAD ~ Intercept.Low + AdipA1 + NTproBNP + PlGF + A8MPO + A12TNF |
| 9 | CAD ~ Intercept.Low + AdipA1 + NTproBNP + PlGF + A8MPO + A12TNF |
| 10 | CAD ~ Intercept.Low + AdipA1 + NTproBNP + PlGF + A8MPO + A12TNF |
| 11 | CAD ~ Intercept.Low + AdipA1 + NTproBNP + PlGF + A8MPO + A12TNF |
| 12 | CAD ~ Intercept.Low + AdipA1 + NTproBNP + PlGF + A8MPO + A12TNF |
| 13 | CAD ~ Intercept.Low + AdipA1 + NTproBNP + PlGF + A8MPO + A12TNF |
| 14 | CAD ~ Intercept.Low + AdipA1 + NTproBNP + PlGF + A8MPO |
| 15 | CAD ~ Intercept.Low + AdipA1 + NTproBNP + PlGF + A12TNF |
| 16 | CAD ~ Intercept.Low + AdipA1 + NTproBNP + A8MPO + A12TNF |
| 17 | CAD ~ Intercept.Low + AdipA1 + PlGF + A8MPO + A12TNF |
| 18 | CAD ~ Intercept.Low + NTproBNP + PlGF + A8MPO + A12TNF |
| 19 | CAD ~ Intercept Low + AdipA1 + NTproBNP + PlGF + A8MPO + A12TNF |
| 20 | CAD ~ Intercept.Low + AdipA1 + NTproBNP + PlGF + A8MPO + A12TNF |
| 21 | CAD ~ Intercept.Low + AdipA1 + NTproBNP + PlGF + A8MPO + A12TNF |
| 22 | CAD ~ Intercept.Low + AdipA1 + NTproBNP + PlGF + A8MPO + A12TNF |
| 23 | CAD ~ Intercept.Low + AdipA1 + NTproBNP + PlGF + A8MPO + A12TNF |
| 24 | CAD ~ Intercept.Low + AdipA1 + NTproBNP + PlGF + A8MPO + A12TNF |
| 25 | CAD ~ Intercept.Low + AdipA1 + NTproBNP + PlGF |
| 26 | CAD ~ Intercept.Low + AdipA1 + NTproBNP + A8MPO |
| 27 | CAD ~ Intercept.Low + AdipA1 + NTproBNP + A12TNF |
| 28 | CAD ~ Intercept.Low + AdipA1 + PlGF + A8MPO |
| 29 | CAD ~ Intercept.Low + AdipA1 + PlGF + A12TNF |
| 30 | CAD ~ Intercept.Low + AdipA1 + A8MPO + A12TNF |
| 31 | CAD ~ Intercept.Low + NTproBNP + PlGF + A8MPO |
| 32 | CAD ~ Intercept.Low + NTproBNP + PlGF + A12TNF |
| 33 | CAD ~ Intercept.Low + NTproBNP + A8MPO + A12TNF |
| 34 | CAD ~ Intercept.Low + PlGF + A8MPO + A12TNF |
| 35 | CAD ~ Intercept.Low + AdipA1 + NTproBNP + PlGF + A8MPO |
| 36 | CAD ~ Intercept.Low + AdipA1 + NTproBNP + PlGF + A12TNF |
| 37 | CAD ~ Intercept.Low + AdipA1 + NTproBNP + A8MPO + A12TNF |
| 38 | CAD ~ Intercept.Low + AdipA1 + PlGF + A8MPO + A12TNF |
| 39 | CAD ~ Intercept.Low + NTproBNP + PlGF + A8MPO + A12TNF |
| 40 | CAD ~ Intercept.Low + AdipA1 + NTproBNP + PlGF + A8MPO |
| 41 | CAD ~ Intercept.Low + AdipA1 + NTproBNP + PlGF + A12TNF |
| 42 | CAD ~ Intercept.Low + AdipA1 + NTproBNP + A8MPO + A12TNF |
| 43 | CAD ~ Intercept.Low + AdipA1 + PlGF + A8MPO + A12TNF |
| 44 | CAD ~ Intercept.Low + NTproBNP + PlGF + A8MPO + A12TNF |
| 45 | CAD ~ Intercept.Low + AdipA1 + NTproBNP + PlGF + A8MPO + A12TNF |
| 46 | CAD ~ Intercept.Low + AdipA1 + NTproBNP + PlGF + A8MPO |
| 47 | CAD ~ Intercept.Low + AdipA1 + NTproBNP + PlGF + A12TNF |
| 48 | CAD ~ Intercept.Low + AdipA1 + NTproBNP + A8MPO + A12TNF |
| 49 | CAD ~ Intercept.Low + AdipA1 + PlGF + A8MPO + A12TNF |
| 50 | CAD ~ Intercept.Low + NTproBNP + PlGF + A8MPO + A12TNF |
| 51 | CAD ~ Intercept.Low + AdipA1 + NTproBNP + PlGF + A8MPO + A12TNF |
| 52 | CAD ~ Intercept.Low + AdipA1 + NTproBNP + PlGF + A8MPO + A12TNF |
| 53 | CAD ~ Intercept.Low + AdipA1 + NTproBNP + PlGF + A8MPO |
| 54 | CAD ~ Intercept.Low + AdipA1 + NTproBNP + PlGF + A12TNF |
| 55 | CAD ~ Intercept.Low + AdipA1 + NTproBNP + A8MPO + A12TNF |
| 56 | CAD ~ Intercept.Low + AdipA1 + PlGF + A8MPO + A12TNF |
| 57 | CAD ~ Intercept.Low + NTproBNP + PlGF + A8MPO + A12TNF |
| 58 | CAD ~ Intercept.Low + AdipA1 + NTproBNP + PlGF + A8MPO + A12TNF |
| 59 | CAD ~ Intercept.Low + AdipA1 + NTproBNP + PlGF + A8MPO + A12TNF |
| 60 | CAD ~ Intercept.Low + AdipA1 + NTproBNP + PlGF + A8MPO + A12TNF |
| 61 | CAD ~ Intercept.Low + AdipA1 + NTproBNP + PlGF + A8MPO |
| 62 | CAD ~ Intercept.Low + AdipA1 + NTproBNP + PlGF + A12TNF |
| 63 | CAD ~ Intercept.Low + AdipA1 + NTproBNP + A8MPO + A12TNF |
| 64 | CAD ~ Intercept.Low + AdipA1 + PlGF + A8MPO + A12TNF |
| 65 | CAD ~ Intercept.Low + NTproBNP + PlGF + A8MPO + A12TNF |
| 66 | CAD ~ Intercept.Low + AdipA1 + NTproBNP + PlGF + A8MPO + A12TNF |
| 67 | CAD ~ Intercept.Low + AdipA1 + NTproBNP + PlGF + A8MPO + A12TNF |
| 68 | CAD ~ Intercept.Low + AdipA1 + NTproBNP + PlGF + A8MPO + A12TNF |
| 69 | CAD ~ Intercept.Low + AdipA1 + NTproBNP + PlGF + A8MPO + A12TNF |
| 70 | CAD ~ Intercept.Low + AdipA1 + NTproBNP + PlGF + A8MPO |
| 71 | CAD ~ Intercept.Low + AdipA1 + NTproBNP + PlGF + A12TNF |
| 72 | CAD ~ Intercept.Low + AdipA1 + NTproBNP + A8MPO + A12TNF |
| 73 | CAD ~ Intercept.Low + AdipA1 + PlGF + A8MPO + A12TNF |
| 74 | CAD ~ Intercept.Low + NTproBNP + PlGF + A8MPO + A12TNF |
| 75 | CAD ~ Intercept.Low + AdipA1 + NTproBNP + PlGF + A8MPO + A12TNF |
| 76 | CAD ~ Intercept.Low + AdipA1 + NTproBNP + PlGF + A8MPO + A12TNF |
| 77 | CAD ~ Intercept.Low + AdipA1 + NTproBNP + PlGF + A8MPO + A12TNF |
| 78 | CAD ~ Intercept.Low + AdipA1 + NTproBNP + PlGF + A8MPO + A12TNF |
| 79 | CAD ~ Intercept.Low + AdipA1 + NTproBNP + PlGF + A8MPO + A12TNF |
| 80 | CAD ~ Intercept.Low + AdipA1 + NTproBNP + PlGF |
| 81 | CAD ~ Intercept.Low + AdipA1 + NTproBNP + A8MPO |
| 82 | CAD ~ Intercept.Low + AdipA1 + NTproBNP + A12TNF |
| 83 | CAD ~ Intercept.Low + AdipA1 + PlGF + A8MPO |
| 84 | CAD ~ Intercept.Low + AdipA1 + PlGF + A12TNF |
| 85 | CAD ~ Intercept.Low + AdipA1 + A8MPO + A12TNF |
| 86 | CAD ~ Intercept.Low + NTproBNP + PlGF + A8MPO |
| 87 | CAD ~ Intercept.Low + NTproBNP + PlGF + A12TNF |
| 88 | CAD ~ Intercept.Low + NTproBNP + A8MPO + A12TNF |
| 89 | CAD ~ Intercept.Low + PlGF + A8MPO + A12TNF |
| 90 | CAD ~ Intercept.Low + AdipA1 + NTproBNP + PlGF + A8MPO |
| 91 | CAD ~ Intercept.Low + AdipA1 + NTproBNP + PlGF + A12TNF |
| 92 | CAD ~ Intercept.Low + AdipA1 + NTproBNP + A8MPO + A12TNF |
| 93 | CAD ~ Intercept.Low + AdipA1 + PlGF + A8MPO + A12TNF |
| 94 | CAD ~ Intercept.Low + NTproBNP + PlGF + A8MPO + A12TNF |
| 95 | CAD ~ Intercept.Low + AdipA1 + NTproBNP + PlGF + A8MPO |
| 96 | CAD ~ Intercept.Low + AdipA1 + NTproBNP + PlGF + A12TNF |
| 97 | CAD ~ Intercept.Low + AdipA1 + NTproBNP + A8MPO + A12TNF |
| 98 | CAD ~ Intercept.Low + AdipA1 + PlGF + A8MPO + A12TNF |
| 99 | CAD ~ Intercept.Low + NTproBNP + PlGF + A8MPO + A12TNF |
| 100 | CAD ~ Intercept.Low + AdipA1 + NTproBNP + PlGF + A8MPO + A12TNF |
| 101 | CAD ~ Intercept.Low + AdipA1 + NTproBNP + PlGF + A8MPO |
| 102 | CAD ~ Intercept.Low + AdipA1 + NTproBNP + PlGF + A12TNF |
| 103 | CAD ~ Intercept.Low + AdipA1 + NTproBNP + A8MPO + A12TNF |
| 104 | CAD ~ Intercept.Low + AdipA1 + PlGF + A8MPO + A12TNF |
| 105 | CAD ~ Intercept.Low + NTproBNP + PlGF + A8MPO + A12TNF |
| 106 | CAD ~ Intercept.Low + AdipA1 + NTproBNP + PlGF + A8MPO + A12TNF |
| 107 | CAD ~ Intercept.Low + AdipA1 + NTproBNP + PlGF + A8MPO + A12TNF |
| 108 | CAD ~ Intercept.Low + AdipA1 + NTproBNP + PlGF + A8MPO |
| 109 | CAD ~ Intercept.Low + AdipA1 + NTproBNP + PlGF + A12TNF |
| 110 | CAD ~ Intercept.Low + AdipA1 + NTproBNP + A8MPO + A12TNF |
| 111 | CAD ~ Intercept.Low + AdipA1 + PlGF + A8MPO + A12TNF |
| 112 | CAD ~ Intercept.Low + NTproBNP + PlGF + A8MPO + A12TNF |
| 113 | CAD ~ Intercept.Low + AdipA1 + NTproBNP + PlGF + A8MPO + A12TNF |
| 114 | CAD ~ Intercept.Low + AdipA1 + NTproBNP + PlGF + A8MPO + A12TNF |
| 115 | CAD ~ Intercept.Low + AdipA1 + NTproBNP + PlGF + A8MPO + A12TNF |
| 116 | CAD ~ Intercept.Low + AdipA1 + NTproBNP + PlGF + A8MPO |
| 117 | CAD ~ Intercept.Low + AdipA1 + NTproBNP + PlGF + A12TNF |
| 118 | CAD ~ Intercept.Low + AdipA1 + NTproBNP + A8MPO + A12TNF |
| 119 | CAD ~ Intercept.Low + AdipA1 + PlGF + A8MPO + A12TNF |
| 120 | CAD ~ Intercept.Low + NTproBNP + PlGF + A8MPO + A12TNF |
| 121 | CAD ~ Intercept.Low + AdipA1 + NTproBNP + PlGF + A8MPO + A12TNF |
| 122 | CAD ~ Intercept.Low + AdipA1 + NTproBNP + PlGF + A8MPO + A12TNF |
| 123 | CAD ~ Intercept.Low + AdipA1 + NTproBNP + PlGF + A8MPO + A12TNF |
| 124 | CAD ~ Intercept.Low + AdipA1 + NTproBNP + PlGF + A8MPO + A12TNF |
| 125 | CAD ~ Intercept.Low + AdipA1 + NTproBNP + PlGF + A8MPO |
| 126 | CAD ~ Intercept.Low + AdipA1 + NTproBNP + PlGF + A12TNF |
| 127 | CAD ~ Intercept.Low + AdipA1 + NTproBNP + A8MPO + A12TNF |
| 128 | CAD ~ Intercept.Low + AdipA1 + PlGF + A8MPO + A12TNF |
| 129 | CAD ~ Intercept.Low + NTproBNP + PlGF + A8MPO + A12TNF |
| 130 | CAD ~ Intercept.Low + AdipA1 + NTproBNP + PlGF + A8MPO + A12TNF |
| 131 | CAD ~ Intercept.Low + AdipA1 + NTproBNP + PlGF + A8MPO + A12TNF |
| 132 | CAD ~ Intercept.Low + AdipA1 + NTproBNP + PlGF + A8MPO + A12TNF |
| 133 | CAD ~ Intercept.Low + AdipA1 + NTproBNP + PlGF + A8MPO + A12TNF |
| 134 | CAD ~ Intercept.Low + AdipA1 + NTproBNP + PlGF + A8MPO + A12TNF |
| 135 | CAD ~ Intercept.Low + A8MPO + A12TNF |
| 136 | CAD ~ Intercept.Low + AdipA1 + NTproBNP |
| 137 | CAD ~ Intercept.Low + AdipA1 + PlGF |
| 138 | CAD ~ Intercept.Low + AdipA1 + A8MPO |
| 139 | CAD ~ Intercept.Low + AdipA1 + A12TNF |
| 140 | CAD ~ Intercept.Low + NTproBNP + PlGF |
| 141 | CAD ~ Intercept.Low + NTproBNP + A8MPO |
| 142 | CAD ~ Intercept.Low + NTproBNP + A12TNF |
| 143 | CAD ~ Intercept.Low + PlGF + A8MPO |
| 144 | CAD ~ Intercept.Low + PlGF + A12TNF |
| 145 | CAD ~ Intercept.Low + AdipA1 + NTproBNP + PlGF |
| 146 | CAD ~ Intercept.Low + AdipA1 + NTproBNP + A8MPO |
| 147 | CAD ~ Intercept.Low + AdipA1 + NTproBNP + A12TNF |
| 148 | CAD ~ Intercept.Low + AdipA1 + PlGF + A8MPO |
| 149 | CAD ~ Intercept.Low + AdipA1 + PlGF + A12TNF |
| 150 | CAD ~ Intercept.Low + AdipA1 + A8MPO + A12TNF |
| 151 | CAD ~ Intercept.Low + NTproBNP + PlGF + A8MPO |
| 152 | CAD ~ Intercept.Low + NTproBNP + PlGF + A12TNF |
| 153 | CAD ~ Intercept.Low + NTproBNP + A8MPO + A12TNF |
| 154 | CAD ~ Intercept.Low + PlGF + A8MPO + A12TNF |
| 155 | CAD ~ Intercept.Low + AdipA1 + NTproBNP + PlGF |
| 156 | CAD ~ Intercept.Low + AdipA1 + NTproBNP + A8MPO |
| 157 | CAD ~ Intercept.Low + AdipA1 + NTproBNP + A12TNF |
| 158 | CAD ~ Intercept.Low + AdipA1 + PlGF + A8MPO |
| 159 | CAD ~ Intercept.Low + AdipA1 + PlGF + A12TNF |
| 160 | CAD ~ Intercept.Low + AdipA1 + A8MPO + A12TNF |
| 161 | CAD ~ Intercept.Low + NTproBNP + PlGF + A8MPO |
| 162 | CAD ~ Intercept.Low + NTproBNP + PlGF + A12TNF |
| 163 | CAD ~ Intercept.Low + NTproBNP + A8MPO + A12TNF |
| 164 | CAD ~ Intercept.Low + PlGF + A8MPO + A12TNF |
| 165 | CAD ~ Intercept.Low + AdipA1 + NTproBNP + PlGF + A8MPO |
| 166 | CAD ~ Intercept.Low + AdipA1 + NTproBNP + PlGF + A12TNF |
| 167 | CAD ~ Intercept.Low + AdipA1 + NTproBNP + A8MPO + A12TNF |
| 168 | CAD ~ Intercept.Low + AdipA1 + PlGF + A8MPO + A12TNF |
| 169 | CAD ~ Intercept.Low + NTproBNP + PlGF + A8MPO + A12TNF |
| 170 | CAD ~ Intercept.Low + AdipA1 + NTproBNP + PlGF |
| 171 | CAD ~ Intercept.Low + AdipA1 + NTproBNP + A8MPO |
| 172 | CAD ~ Intercept.Low + AdipA1 + NTproBNP + A12TNF |
| 173 | CAD ~ Intercept.Low + AdipA1 + PlGF + A8MPO |
| 174 | CAD ~ Intercept.Low + AdipA1 + PlGF + A12TNF |
| 175 | CAD ~ Intercept.Low + AdipA1 + A8MPO + A12TNF |
| 176 | CAD ~ Intercept.Low + NTproBNP + PlGF + A8MPO |
| 177 | CAD ~ Intercept.Low + NTproBNP + PlGF + A12TNF |
| 178 | CAD ~ Intercept.Low + NTproBNP + A8MPO + A12TNF |
| 179 | CAD ~ Intercept.Low + PlGF + A8MPO + A12TNF |
| 180 | CAD ~ Intercept.Low + AdipA1 + NTproBNP + PlGF + A8MPO |
| 181 | CAD ~ Intercept.Low + AdipA1 + NTproBNP + PlGF + A12TNF |
| 182 | CAD ~ Intercept.Low + AdipA1 + NTproBNP + A8MPO + A12TNF |
| 183 | CAD ~ Intercept.Low + AdipA1 + PlGF + A8MPO + A12TNF |
| 184 | CAD ~ Intercept.Low + NTproBNP + PlGF + A8MPO + A12TNF |
| 185 | CAD ~ Intercept.Low + AdipA1 + NTproBNP + PlGF + A8MPO |
| 186 | CAD ~ Intercept.Low + AdipA1 + NTproBNP + PlGF + A12TNF |
| 187 | CAD ~ Intercept.Low + AdipA1 + NTproBNP + A8MPO + A12TNF |
| 188 | CAD ~ Intercept.Low + AdipA1 + PlGF + A8MPO + A12TNF |
| 189 | CAD ~ Intercept.Low + NTproBNP + PlGF + A8MPO + A12TNF |
| 190 | CAD ~ Intercept.Low + AdipA1 + NTproBNP + PlGF + A8MPO + A12TNF |
| 191 | CAD ~ Intercept.Low + AdipA1 + NTproBNP + PlGF |
| 192 | CAD ~ Intercept.Low + AdipA1 + NTproBNP + A8MPO |
| 193 | CAD ~ Intercept.Low + AdipA1 + NTproBNP + A12TNF |
| 194 | CAD ~ Intercept.Low + AdipA1 + PlGF + A8MPO |
| 195 | CAD ~ Intercept.Low + AdipA1 + PlGF + A12TNF |
| 196 | CAD ~ Intercept.Low + AdipA1 + A8MPO + A12TNF |
| 197 | CAD ~ Intercept.Low + NTproBNP + PlGF + A8MPO |
| 198 | CAD ~ Intercept.Low + NTproBNP + PlGF + A12TNF |
| 199 | CAD ~ Intercept.Low + NTproBNP + A8MPO + A12TNF |
| 200 | CAD ~ Intercept.Low + PlGF + A8MPO + A12TNF |
| 201 | CAD ~ Intercept.Low + AdipA1 + NTproBNP + PlGF + A8MPO |
| 202 | CAD ~ Intercept.Low + AdipA1 + NTproBNP + PlGF + A12TNF |
| 203 | CAD ~ Intercept.Low + AdipA1 + NTproBNP + A8MPO + A12TNF |
| 204 | CAD ~ Intercept.Low + AdipA1 + PlGF + A8MPO + A12TNF |
| 205 | CAD ~ Intercept.Low + NTproBNP + PlGF + A8MPO + A12TNF |
| 206 | CAD ~ Intercept.Low + AdipA1 + NTproBNP + PlGF + A8MPO |
| 207 | CAD ~ Intercept.Low + AdipA1 + NTproBNP + PlGF + A12TNF |
| 208 | CAD ~ Intercept.Low + AdipA1 + NTproBNP + A8MPO + A12TNF |
| 209 | CAD ~ Intercept.Low + AdipA1 + PlGF + A8MPO + A12TNF |
| 210 | CAD ~ Intercept.Low + NTproBNP + PlGF + A8MPO + A12TNF |
| 211 | CAD ~ Intercept.Low + AdipA1 + NTproBNP + PlGF + A8MPO + A12TNF |
| 212 | CAD ~ Intercept.Low + AdipA1 + NTproBNP + PlGF + A8MPO |
| 213 | CAD ~ Intercept.Low + AdipA1 + NTproBNP + PlGF + A12TNF |
| 214 | CAD ~ Intercept.Low + AdipA1 + NTproBNP + A8MPO + A12TNF |
| 215 | CAD ~ Intercept.Low + AdipA1 + PlGF + A8MPO + A12TNF |
| 216 | CAD ~ Intercept.Low + NTproBNP + PlGF + A8MPO + A12TNF |
| 217 | CAD ~ Intercept.Low + AdipA1 + NTproBNP + PlGF + A8MPO + A12TNF |
| 218 | CAD ~ Intercept.Low + AdipA1 + NTproBNP + PlGF + A8MPO + A12TNF |
| 219 | CAD ~ Intercept.Low + AdipA1 + NTproBNP + PlGF |
| 220 | CAD ~ Intercept.Low + AdipA1 + NTproBNP + A8MPO |
| 221 | CAD ~ Intercept.Low + AdipA1 + NTproBNP + A12TNF |
| 222 | CAD ~ Intercept.Low + AdipA1 + PlGF + A8MPO |
| 223 | CAD ~ Intercept.Low + AdipA1 + PlGF + A12TNF |
| 224 | CAD ~ Intercept.Low + AdipA1 + A8MPO + A12TNF |
| 225 | CAD ~ Intercept.Low + NTproBNP + PlGF + A8MPO |
| 226 | CAD ~ Intercept.Low + NTproBNP + PlGF + A12TNF |
| 227 | CAD ~ Intercept.Low + NTproBNP + A8MPO + A12TNF |
| 228 | CAD ~ Intercept.Low + PlGF + A8MPO + A12TNF |
| 229 | CAD ~ Intercept.Low + AdipA1 + NTproBNP + PlGF + A8MPO |
| 230 | CAD ~ Intercept.Low + AdipA1 + NTproBNP + PlGF + A12TNF |
| 231 | CAD ~ Intercept.Low + AdipA1 + NTproBNP + A8MPO + A12TNF |
| 232 | CAD ~ Intercept.Low + AdipA1 + PlGF + A8MPO + A12TNF |
| 233 | CAD ~ Intercept.Low + NTproBNP + PlGF + A8MPO + A12TNF |
| 234 | CAD ~ Intercept.Low + AdipA1 + NTproBNP + PlGF + A8MPO |
| 235 | CAD ~ Intercept.Low + AdipA1 + NTproBNP + PlGF + A12TNF |
| 236 | CAD ~ Intercept.Low + AdipA1 + NTproBNP + A8MPO + A12TNF |
| 237 | CAD ~ Intercept.Low + AdipA1 + PlGF + A8MPO + A12TNF |
| 238 | CAD ~ Intercept.Low + NTproBNP + PlGF + A8MPO + A12TNF |
| 239 | CAD ~ Intercept.Low + AdipA1 + NTproBNP + PlGF + A8MPO + A12TNF |
| 240 | CAD ~ Intercept.Low + AdipA1 + NTproBNP + PlGF + A8MPO |
| 241 | CAD ~ Intercept.Low + AdipA1 + NTproBNP + PlGF + A12TNF |
| 242 | CAD ~ Intercept.Low + AdipA1 + NTproBNP + A8MPO + A12TNF |
| 243 | CAD ~ Intercept.Low + AdipA1 + PlGF + A8MPO + A12TNF |
| 244 | CAD ~ Intercept.Low + NTproBNP + PlGF + A8MPO + A12TNF |
| 245 | CAD ~ Intercept.Low + AdipA1 + NTproBNP + PlGF + A8MPO + A12TNF |
| 246 | CAD ~ Intercept.Low + AdipA1 + NTproBNP + PlGF + A8MPO + A12TNF |
| 247 | CAD ~ Intercept.Low + AdipA1 + NTproBNP + PlGF + A8MPO |
| 248 | CAD ~ Intercept.Low + AdipA1 + NTproBNP + PlGF + A12TNF |
| 249 | CAD ~ Intercept.Low + AdipA1 + NTproBNP + A8MPO + A12TNF |
| 250 | CAD ~ Intercept.Low + AdipA1 + PlGF + A8MPO + A12TNF |
| 251 | CAD ~ Intercept.Low + NTproBNP + PlGF + A8MPO + A12TNF |
| 252 | CAD ~ Intercept.Low + AdipA1 + NTproBNP + PlGF + A8MPO + A12TNF |
| 253 | CAD ~ Intercept.Low + AdipA1 + NTproBNP + PlGF + A8MPO + A12TNF |
| 254 | CAD ~ Intercept.Low + AdipA1 + NTproBNP + PlGF + A8MPO + A12TNF |
| 255 | CAD ~ Intercept.Low + AdipA1 + NTproBNP + PlGF |
| 256 | CAD ~ Intercept.Low + AdipA1 + NTproBNP + A8MPO |
| 257 | CAD ~ Intercept.Low + AdipA1 + NTproBNP + A12TNF |
| 258 | CAD ~ Intercept.Low + AdipA1 + PlGF + A8MPO |
| 259 | CAD ~ Intercept.Low + AdipA1 + PlGF + A12TNF |
| 260 | CAD ~ Intercept.Low + AdipA1 + A8MPO + A12TNF |
| 261 | CAD ~ Intercept.Low + NTproBNP + PlGF + A8MPO |
| 262 | CAD ~ Intercept.Low + NTproBNP + PlGF + A12TNF |
| 263 | CAD ~ Intercept.Low + NTproBNP + A8MPO + A12TNF |
| 264 | CAD ~ Intercept.Low + PlGF + A8MPO + A12TNF |
| 265 | CAD ~ Intercept.Low + AdipA1 + NTproBNP + PlGF + A8MPO |
| 266 | CAD ~ Intercept.Low + AdipA1 + NTproBNP + PlGF + A12TNF |
| 267 | CAD ~ Intercept.Low + AdipA1 + NTproBNP + A8MPO + A12TNF |
| 268 | CAD ~ Intercept.Low + AdipA1 + PlGF + A8MPO + A12TNF |
| 269 | CAD ~ Intercept.Low + NTproBNP + PlGF + A8MPO + A12TNF |
| 270 | CAD ~ Intercept.Low + AdipA1 + NTproBNP + PlGF + A8MPO |
| 271 | CAD ~ Intercept.Low + AdipA1 + NTproBNP + PlGF + A12TNF |
| 272 | CAD ~ Intercept.Low + AdipA1 + NTproBNP + A8MPO + A12TNF |
| 273 | CAD ~ Intercept.Low + AdipA1 + PlGF + A8MPO + A12TNF |
| 274 | CAD ~ Intercept.Low + NTproBNP + PlGF + A8MPO + A12TNF |
| 275 | CAD ~ Intercept.Low + AdipA1 + NTproBNP + PlGF + A8MPO + A12TNF |
| 276 | CAD ~ Intercept.Low + AdipA1 + NTproBNP + PlGF + A8MPO |
| 277 | CAD ~ Intercept.Low + AdipA1 + NTproBNP + PlGF + A12TNF |
| 278 | CAD ~ Intercept.Low + AdipA1 + NTproBNP + A8MPO + A12TNF |
| 279 | CAD ~ Intercept.Low + AdipA1 + PlGF + A8MPO + A12TNF |
| 280 | CAD ~ Intercept.Low + NTproBNP + PlGF + A8MPO + A12TNF |
| 281 | CAD ~ Intercept.Low + AdipA1 + NTproBNP + PlGF + A8MPO + A12TNF |
| 282 | CAD ~ Intercept.Low + AdipA1 + NTproBNP + PlGF + A8MPO + A12TNF |
| 283 | CAD ~ Intercept.Low + AdipA1 + NTproBNP + PlGF + A8MPO |
| 284 | CAD ~ Intercept.Low + AdipA1 + NTproBNP + PlGF + A12TNF |
| 285 | CAD ~ Intercept.Low + AdipA1 + NTproBNP + A8MPO + A12TNF |
| 286 | CAD ~ Intercept.Low + AdipA1 + PlGF + A8MPO + A12TNF |
| 287 | CAD ~ Intercept.Low + NTproBNP + PlGF + A8MPO + A12TNF |
| 288 | CAD ~ Intercept.Low + AdipA1 + NTproBNP + PlGF + A8MPO + A12TNF |
| 289 | CAD ~ Intercept.Low + AdipA1 + NTproBNP + PlGF + A8MPO + A12TNF |
| 290 | CAD ~ Intercept.Low + AdipA1 + NTproBNP + PlGF + A8MPO + A12TNF |
| 291 | CAD ~ Intercept.Low + AdipA1 + NTproBNP + PlGF + A8MPO |
| 292 | CAD ~ Intercept.Low + AdipA1 + NTproBNP + PlGF + A12TNF |
| 293 | CAD ~ Intercept.Low + AdipA1 + NTproBNP + A8MPO + A12TNF |
| 294 | CAD ~ Intercept.Low + AdipA1 + PlGF + A8MPO + A12TNF |
| 295 | CAD ~ Intercept.Low + NTproBNP + PlGF + A8MPO + A12TNF |
| 296 | CAD ~ Intercept.Low + AdipA1 + NTproBNP + PlGF + A8MPO + A12TNF |
| 297 | CAD ~ Intercept.Low + AdipA1 + NTproBNP + PlGF + A8MPO + A12TNF |
| 298 | CAD ~ Intercept.Low + AdipA1 + NTproBNP + PlGF + A8MPO + A12TNF |
| 299 | CAD ~ Intercept.Low + AdipA1 + NTproBNP + PlGF + A8MPO + A12TNF |
| 300 | CAD ~ Intercept.Low + A8MPO + A12TNF |
| 301 | CAD ~ Intercept.Low + AdipA1 + NTproBNP |
| 302 | CAD ~ Intercept.Low + AdipA1 + PlGF |
| 303 | CAD ~ Intercept.Low + AdipA1 + A8MPO |
| 304 | CAD ~ Intercept.Low + AdipA1 + A12TNF |
| 305 | CAD ~ Intercept.Low + NTproBNP + PlGF |
| 306 | CAD ~ Intercept.Low + NTproBNP + A8MPO |
| 307 | CAD ~ Intercept.Low + NTproBNP + A12TNF |
| 308 | CAD ~ Intercept.Low + PlGF + A8MPO |
| 309 | CAD ~ Intercept.Low + PlGF + A12TNF |
| 310 | CAD ~ Intercept.Low + AdipA1 + NTproBNP + PlGF |
| 311 | CAD ~ Intercept.Low + AdipA1 + NTproBNP + A8MPO |
| 312 | CAD ~ Intercept.Low + AdipA1 + NTproBNP + A12TNF |
| 313 | CAD ~ Intercept.Low + AdipA1 + PlGF + A8MPO |
| 314 | CAD ~ Intercept.Low + AdipA1 + PlGF + A12TNF |
| 315 | CAD ~ Intercept.Low + AdipA1 + A8MPO + A12TNF |
| 316 | CAD ~ Intercept.Low + NTproBNP + PlGF + A8MPO |
| 317 | CAD ~ Intercept.Low + NTproBNP + PlGF + A12TNF |
| 318 | CAD ~ Intercept.Low + NTproBNP + A8MPO + A12TNF |
| 319 | CAD ~ Intercept.Low + PlGF + A8MPO + A12TNF |
| 320 | CAD ~ Intercept.Low + AdipA1 + NTproBNP + PlGF |
| 321 | CAD ~ Intercept.Low + AdipA1 + NTproBNP + A8MPO |
| 322 | CAD ~ Intercept.Low + AdipA1 + NTproBNP + A12TNF |
| 323 | CAD ~ Intercept.Low + AdipA1 + PlGF + A8MPO |
| 324 | CAD ~ Intercept.Low + AdipA1 + PlGF + A12TNF |
| 325 | CAD ~ Intercept.Low + AdipA1 + A8MPO + A12TNF |
| 326 | CAD ~ Intercept.Low + NTproBNP + PlGF + A8MPO |
| 327 | CAD ~ Intercept.Low + NTproBNP + PlGF + A12TNF |
| 328 | CAD ~ Intercept.Low + NTproBNP + A8MPO + A12TNF |
| 329 | CAD ~ Intercept.Low + PlGF + A8MPO + A12TNF |
| 330 | CAD ~ Intercept.Low + AdipA1 + NTproBNP + PlGF + A8MPO |
| 331 | CAD ~ Intercept.Low + AdipA1 + NTproBNP + PlGF + A12TNF |
| 332 | CAD ~ Intercept.Low + AdipA1 + NTproBNP + A8MPO + A12TNF |
| 333 | CAD ~ Intercept.Low + AdipA1 + PlGF + A8MPO + A12TNF |
| 334 | CAD ~ Intercept.Low + NTproBNP + PlGF + A8MPO + A12TNF |
| 335 | CAD ~ Intercept.Low + AdipA1 + NTproBNP + PlGF |
| 336 | CAD ~ Intercept.Low + AdipA1 + NTproBNP + A8MPO |
| 337 | CAD ~ Intercept.Low + AdipA1 + NTproBNP + A12TNF |
| 338 | CAD ~ Intercept.Low + AdipA1 + PlGF + A8MPO |
| 339 | CAD ~ Intercept.Low + AdipA1 + PlGF + A12TNF |
| 340 | CAD ~ Intercept.Low + AdipA1 + A8MPO + A12TNF |
| 341 | CAD ~ Intercept.Low + NTproBNP + PlGF + A8MPO |
| 342 | CAD ~ Intercept.Low + NTproBNP + PlGF + A12TNF |
| 343 | CAD ~ Intercept.Low + NTproBNP + A8MPO + A12TNF |
| 344 | CAD ~ Intercept.Low + PlGF + A8MPO + A12TNF |
| 345 | CAD ~ Intercept.Low + AdipA1 + NTproBNP + PlGF + A8MPO |
| 346 | CAD ~ Intercept.Low + AdipA1 + NTproBNP + PlGF + A12TNF |
| 347 | CAD ~ Intercept.Low + AdipA1 + NTproBNP + A8MPO + A12TNF |
| 348 | CAD ~ Intercept.Low + AdipA1 + PlGF + A8MPO + A12TNF |
| 349 | CAD ~ Intercept.Low + NTproBNP + PlGF + A8MPO + A12TNF |
| 350 | CAD ~ Intercept.Low + AdipA1 + NTproBNP + PlGF + A8MPO |
| 351 | CAD ~ Intercept.Low + AdipA1 + NTproBNP + PlGF + A12TNF |
| 352 | CAD ~ Intercept.Low + AdipA1 + NTproBNP + A8MPO + A12TNF |
| 353 | CAD ~ Intercept.Low + AdipA1 + PlGF + A8MPO + A12TNF |
| 354 | CAD ~ Intercept.Low + NTproBNP + PlGF + A8MPO + A12TNF |
| 355 | CAD ~ Intercept.Low + AdipA1 + NTproBNP + PlGF + A8MPO + A12TNF |
| 356 | CAD ~ Intercept.Low + AdipA1 + NTproBNP + PlGF |
| 357 | CAD ~ Intercept.Low + AdipA1 + NTproBNP + A8MPO |
| 358 | CAD ~ Intercept.Low + AdipA1 + NTproBNP + A12TNF |
| 359 | CAD ~ Intercept.Low + AdipA1 + PlGF + A8MPO |
| 360 | CAD ~ Intercept.Low + AdipA1 + PlGF + A12TNF |
| 361 | CAD ~ Intercept.Low + AdipA1 + A8MPO + A12TNF |
| 362 | CAD ~ Intercept.Low + NTproBNP + PlGF + A8MPO |
| 363 | CAD ~ Intercept.Low + NTproBNP + PlGF + A12TNF |
| 364 | CAD ~ Intercept.Low + NTproBNP + A8MPO + A12TNF |
| 365 | CAD ~ Intercept.Low + PlGF + A8MPO + A12TNF |
| 366 | CAD ~ Intercept.Low + AdipA1 + NTproBNP + PlGF + A8MPO |
| 367 | CAD ~ Intercept.Low + AdipA1 + NTproBNP + PlGF + A12TNF |
| 368 | CAD ~ Intercept.Low + AdipA1 + NTproBNP + A8MPO + A12TNF |
| 369 | CAD ~ Intercept.Low + AdipA1 + PlGF + A8MPO + A12TNF |
| 370 | CAD ~ Intercept.Low + NTproBNP + PlGF + A8MPO + A12TNF |
| 371 | CAD ~ Intercept.Low + AdipA1 + NTproBNP + PlGF + A8MPO |
| 372 | CAD ~ Intercept.Low + AdipA1 + NTproBNP + PlGF + A12TNF |
| 373 | CAD ~ Intercept.Low + AdipA1 + NTproBNP + A8MPO + A12TNF |
| 374 | CAD ~ Intercept.Low + AdipA1 + PlGF + A8MPO + A12TNF |
| 375 | CAD ~ Intercept.Low + NTproBNP + PlGF + A8MPO + A12TNF |
| 376 | CAD ~ Intercept.Low + AdipA1 + NTproBNP + PlGF + A8MPO + A12TNF |
| 377 | CAD ~ Intercept.Low + AdipA1 + NTproBNP + PlGF + A8MPO |
| 378 | CAD ~ Intercept.Low + AdipA1 + NTproBNP + PlGF + A12TNF |
| 379 | CAD ~ Intercept.Low + AdipA1 + NTproBNP + A8MPO + A12TNF |
| 380 | CAD ~ Intercept.Low + AdipA1 + PlGF + A8MPO + A12TNF |
| 381 | CAD ~ Intercept.Low + NTproBNP + PlGF + A8MPO + A12TNF |
| 382 | CAD ~ Intercept.Low + AdipA1 + NTproBNP + PlGF + A8MPO + A12TNF |
| 383 | CAD ~ Intercept.Low + AdipA1 + NTproBNP + PlGF + A8MPO + A12TNF |
| 384 | CAD ~ Intercept.Low + AdipA1 + NTproBNP + PlGF |
| 385 | CAD ~ Intercept.Low + AdipA1 + NTproBNP + A8MPO |
| 386 | CAD ~ Intercept.Low + AdipA1 + NTproBNP + A12TNF |
| 387 | CAD ~ Intercept.Low + AdipA1 + PlGF + A8MPO |
| 388 | CAD ~ Intercept.Low + AdipA1 + PlGF + A12TNF |
| 389 | CAD ~ Intercept.Low + AdipA1 + A8MPO + A12TNF |
| 390 | CAD ~ Intercept.Low + NTproBNP + PlGF + A8MPO |
| 391 | CAD ~ Intercept.Low + NTproBNP + PlGF + A12TNF |
| 392 | CAD ~ Intercept.Low + NTproBNP + A8MPO + A12TNF |
| 393 | CAD ~ Intercept.Low + PlGF + A8MPO + A12TNF |
| 394 | CAD ~ Intercept.Low + AdipA1 + NTproBNP + PlGF + A8MPO |
| 395 | CAD ~ Intercept.Low + AdipA1 + NTproBNP + PlGF + A12TNF |
| 396 | CAD ~ Intercept.Low + AdipA1 + NTproBNP + A8MPO + A12TNF |
| 397 | CAD ~ Intercept.Low + AdipA1 + PlGF + A8MPO + A12TNF |
| 398 | CAD ~ Intercept.Low + NTproBNP + PlGF + A8MPO + A12TNF |
| 399 | CAD ~ Intercept.Low + AdipA1 + NTproBNP + PlGF + A8MPO |
| 400 | CAD ~ Intercept.Low + AdipA1 + NTproBNP + PlGF + A12TNF |
| 401 | CAD ~ Intercept.Low + AdipA1 + NTproBNP + A8MPO + A12TNF |
| 402 | CAD ~ Intercept.Low + AdipA1 + PlGF + A8MPO + A12TNF |
| 403 | CAD ~ Intercept.Low + NTproBNP + PlGF + A8MPO + A12TNF |
| 404 | CAD ~ Intercept.Low + AdipA1 + NTproBNP + PlGF + A8MPO + A12TNF |
| 405 | CAD ~ Intercept.Low + AdipA1 + NTproBNP + PlGF + A8MPO |
| 406 | CAD ~ Intercept.Low + AdipA1 + NTproBNP + PlGF + A12TNF |
| 407 | CAD ~ Intercept.Low + AdipA1 + NTproBNP + A8MPO + A12TNF |
| 408 | CAD ~ Intercept.Low + AdipA1 + PlGF + A8MPO + A12TNF |
| 409 | CAD ~ Intercept.Low + NTproBNP + PlGF + A8MPO + A12TNF |
| 410 | CAD ~ Intercept.Low + AdipA1 + NTproBNP + PlGF + A8MPO + A12TNF |
| 411 | CAD ~ Intercept.Low + AdipA1 + NTproBNP + PlGF + A8MPO + A12TNF |
| 412 | CAD ~ Intercept.Low + AdipA1 + NTproBNP + PlGF + A8MPO |
| 413 | CAD ~ Intercept.Low + AdipA1 + NTproBNP + PlGF + A12TNF |
| 414 | CAD ~ Intercept.Low + AdipA1 + NTproBNP + A8MPO + A12TNF |
| 415 | CAD ~ Intercept.Low + AdipA1 + PlGF + A8MPO + A12TNF |
| 416 | CAD ~ Intercept.Low + NTproBNP + PlGF + A8MPO + A12TNF |
| 417 | CAD ~ Intercept.Low + AdipA1 + NTproBNP + PlGF + A8MPO + A12TNF |
| 418 | CAD ~ Intercept.Low + AdipA1 + NTproBNP + PlGF + A8MPO + A12TNF |
| 419 | CAD ~ Intercept.Low + AdipA1 + NTproBNP + PlGF + A8MPO + A12TNF |
| 420 | CAD ~ Intercept.Low + AdipA1 + NTproBNP + PlGF |
| 421 | CAD ~ Intercept.Low + AdipA1 + NTproBNP + A8MPO |
| 422 | CAD ~ Intercept.Low + AdipA1 + NTproBNP + A12TNF |
| 423 | CAD ~ Intercept.Low + AdipA1 + PlGF + A8MPO |
| 424 | CAD ~ Intercept.Low + AdipA1 + PlGF + A12TNF |
| 425 | CAD ~ Intercept.Low + AdipA1 + A8MPO + A12TNF |
| 426 | CAD ~ Intercept.Low + NTproBNP + PlGF + A8MPO |
| 427 | CAD ~ Intercept.Low + NTproBNP + PlGF + A12TNF |
| 428 | CAD ~ Intercept.Low + NTproBNP + A8MPO + A12TNF |
| 429 | CAD ~ Intercept.Low + PlGF + A8MPO + A12TNF |
| 430 | CAD ~ Intercept.Low + AdipA1 + NTproBNP + PlGF + A8MPO |
| 431 | CAD ~ Intercept.Low + AdipA1 + NTproBNP + PlGF + A12TNF |
| 432 | CAD ~ Intercept.Low + AdipA1 + NTproBNP + A8MPO + A12TNF |
| 433 | CAD ~ Intercept.Low + AdipA1 + PlGF + A8MPO + A12TNF |
| 434 | CAD ~ Intercept.Low + NTproBNP + PlGF + A8MPO + A12TNF |
| 435 | CAD ~ Intercept.Low + AdipA1 + NTproBNP + PlGF + A8MPO |
| 436 | CAD ~ Intercept.Low + AdipA1 + NTproBNP + PlGF + A12TNF |
| 437 | CAD ~ Intercept.Low + AdipA1 + NTproBNP + A8MPO + A12TNF |
| 438 | CAD ~ Intercept.Low + AdipA1 + PlGF + A8MPO + A12TNF |
| 439 | CAD ~ Intercept.Low + NTproBNP + PlGF + A8MPO + A12TNF |
| 440 | CAD ~ Intercept.Low + AdipA1 + NTproBNP + PlGF + A8MPO + A12TNF |
| 441 | CAD ~ Intercept.Low + AdipA1 + NTproBNP + PlGF + A8MPO |
| 442 | CAD ~ Intercept.Low + AdipA1 + NTproBNP + PlGF + A12TNF |
| 443 | CAD ~ Intercept.Low + AdipA1 + NTproBNP + A8MPO + A12TNF |
| 444 | CAD ~ Intercept.Low + AdipA1 + PlGF + A8MPO + A12TNF |
| 445 | CAD ~ Intercept.Low + NTproBNP + PlGF + A8MPO + A12TNF |
| 446 | CAD ~ Intercept.Low + AdipA1 + NTproBNP + PlGF + A8MPO + A12TNF |
| 447 | CAD ~ Intercept.Low + AdipA1 + NTproBNP + PlGF + A8MPO + A12TNF |
| 448 | CAD ~ Intercept.Low + AdipA1 + NTproBNP + PlGF + A8MPO |
| 449 | CAD ~ Intercept.Low + AdipA1 + NTproBNP + PlGF + A12TNF |
| 450 | CAD ~ Intercept.Low + AdipA1 + NTproBNP + A8MPO + A12TNF |
| 451 | CAD ~ Intercept.Low + AdipA1 + PlGF + A8MPO + A12TNF |
| 452 | CAD ~ Intercept.Low + NTproBNP + PlGF + A8MPO + A12TNF |
| 453 | CAD ~ Intercept.Low + AdipA1 + NTproBNP + PlGF + A8MPO + A12TNF |
| 454 | CAD ~ Intercept.Low + AdipA1 + NTproBNP + PlGF + A8MPO + A12TNF |
| 455 | CAD ~ Intercept.Low + AdipA1 + NTproBNP + PlGF + A8MPO + A12TNF |
| 456 | CAD ~ Intercept.Low + AdipA1 + NTproBNP + PlGF + A8MPO |
| 457 | CAD ~ Intercept.Low + AdipA1 + NTproBNP + PlGF + A12TNF |
| 458 | CAD ~ Intercept.Low + AdipA1 + NTproBNP + A8MPO + A12TNF |
| 459 | CAD ~ Intercept.Low + AdipA1 + PlGF + A8MPO + A12TNF |
| 460 | CAD ~ Intercept.Low + NTproBNP + PlGF + A8MPO + A12TNF |
| 461 | CAD ~ Intercept.Low + AdipA1 + NTproBNP + PlGF + A8MPO + A12TNF |
| 462 | CAD ~ Intercept.Low + AdipA1 + NTproBNP + PlGF + A8MPO + A12TNF |
| 463 | CAD ~ Intercept.Low + AdipA1 + NTproBNP + PlGF + A8MPO + A12TNF |
| 464 | CAD ~ Intercept.Low + NTproBNP |
| 465 | CAD ~ Intercept.Low + AdipA1 |
| 466 | CAD ~ Intercept.Low + PlGF |
| 467 | CAD ~ Intercept.Low + A8MPO |
| 468 | CAD ~ Intercept.Low + A12TNF |
| 469 | CAD ~ Intercept.Low + A8MPO + A12TNF |
| 470 | CAD ~ Intercept.Low + AdipA1 + NTproBNP |
| 471 | CAD ~ Intercept.Low + AdipA1 + PlGF |
| 472 | CAD ~ Intercept.Low + AdipA1 + A8MPO |
| 473 | CAD ~ Intercept.Low + NTproBNP + PlGF |
| 474 | CAD ~ Intercept.Low + AdipA1 + A12TNF |
| 475 | CAD ~ Intercept.Low + NTproBNP + A8MPO |
| 476 | CAD ~ Intercept.Low + NTproBNP + A12TNF |
| 477 | CAD ~ Intercept.Low + PlGF + A8MPO |
| 478 | CAD ~ Intercept.Low + PlGF + A12TNF |
| 479 | CAD ~ Intercept.Low + A8MPO + A12TNF |
| 480 | CAD ~ Intercept.Low + AdipA1 + NTproBNP |
| 481 | CAD ~ Intercept.Low + AdipA1 + PlGF |
| 482 | CAD ~ Intercept.Low + AdipA1 + A8MPO |
| 483 | CAD ~ Intercept.Low + NTproBNP + PlGF |
| 484 | CAD ~ Intercept.Low + AdipA1 + A12TNF |
| 485 | CAD ~ Intercept.Low + NTproBNP + A8MPO |
| 486 | CAD ~ Intercept.Low + NTproBNP + A12TNF |
| 487 | CAD ~ Intercept.Low + PlGF + A8MPO |
| 488 | CAD ~ Intercept.Low + PlGF + A12TNF |
| 489 | CAD ~ Intercept.Low + AdipA1 + NTproBNP + PlGF |
| 490 | CAD ~ Intercept.Low + AdipA1 + NTproBNP + A8MPO |
| 491 | CAD ~ Intercept.Low + AdipA1 + NTproBNP + A12TNF |
| 492 | CAD ~ Intercept.Low + AdipA1 + PlGF + A8MPO |
| 493 | CAD ~ Intercept.Low + AdipA1 + PlGF + A12TNF |
| 494 | CAD ~ Intercept.Low + AdipA1 + A8MPO + A12TNF |
| 495 | CAD ~ Intercept.Low + NTproBNP + PlGF + A8MPO |
| 496 | CAD ~ Intercept.Low + NTproBNP + PlGF + A12TNF |
| 497 | CAD ~ Intercept.Low + NTproBNP + A8MPO + A12TNF |
| 498 | CAD ~ Intercept.Low + PlGF + A8MPO + A12TNF |
| 499 | CAD ~ Intercept.Low + A8MPO + A12TNF |
| 500 | CAD ~ Intercept.Low + AdipA1 + NTproBNP |
| 501 | CAD ~ Intercept.Low + AdipA1 + PlGF |
| 502 | CAD ~ Intercept.Low + AdipA1 + A8MPO |
| 503 | CAD ~ Intercept.Low + NTproBNP + PlGF |
| 504 | CAD ~ Intercept.Low + AdipA1 + A12TNF |
| 505 | CAD ~ Intercept.Low + NTproBNP + A8MPO |
| 506 | CAD ~ Intercept.Low + NTproBNP + A12TNF |
| 507 | CAD ~ Intercept.Low + PlGF + A8MPO |
| 508 | CAD ~ Intercept.Low + PlGF + A12TNF |
| 509 | CAD ~ Intercept.Low + AdipA1 + NTproBNP + PlGF |
| 510 | CAD ~ Intercept.Low + AdipA1 + NTproBNP + A8MPO |
| 511 | CAD ~ Intercept.Low + AdipA1 + NTproBNP + A12TNF |
| 512 | CAD ~ Intercept.Low + AdipA1 + PlGF + A8MPO |
| 513 | CAD ~ Intercept.Low + AdipA1 + PlGF + A12TNF |
| 514 | CAD ~ Intercept.Low + AdipA1 + A8MPO + A12TNF |
| 515 | CAD ~ Intercept.Low + NTproBNP + PlGF + A8MPO |
| 516 | CAD ~ Intercept.Low + NTproBNP + PlGF + A12TNF |
| 517 | CAD ~ Intercept.Low + NTproBNP + A8MPO + A12TNF |
| 518 | CAD ~ Intercept.Low + PlGF + A8MPO + A12TNF |
| 519 | CAD ~ Intercept.Low + AdipA1 + NTproBNP + PlGF |
| 520 | CAD ~ Intercept.Low + AdipA1 + NTproBNP + A8MPO |
| 521 | CAD ~ Intercept.Low + AdipA1 + NTproBNP + A12TNF |
| 522 | CAD ~ Intercept.Low + AdipA1 + PlGF + A8MPO |
| 523 | CAD ~ Intercept.Low + AdipA1 + PlGF + A12TNF |
| 524 | CAD ~ Intercept.Low + AdipA1 + A8MPO + A12TNF |
| 525 | CAD ~ Intercept.Low + NTproBNP + PlGF + A8MPO |
| 526 | CAD ~ Intercept.Low + NTproBNP + PlGF + A12TNF |
| 527 | CAD ~ Intercept.Low + NTproBNP + A8MPO + A12TNF |
| 528 | CAD ~ Intercept.Low + PlGF + A8MPO + A12TNF |
| 529 | CAD ~ Intercept.Low + AdipA1 + NTproBNP + PlGF + A8MPO |
| 530 | CAD ~ Intercept.Low + AdipA1 + NTproBNP + PlGF + A12TNF |
| 531 | CAD ~ Intercept.Low + AdipA1 + NTproBNP + A8MPO + A12TNF |
| 532 | CAD ~ Intercept.Low + AdipA1 + PlGF + A8MPO + A12TNF |
| 533 | CAD ~ Intercept.Low + NTproBNP + PlGF + A8MPO + A12TNF |
| 534 | CAD ~ Intercept.Low + A8MPO + A12TNF |
| 535 | CAD ~ Intercept.Low + AdipA1 + NTproBNP |
| 536 | CAD ~ Intercept.Low + AdipA1 + PlGF |
| 537 | CAD ~ Intercept.Low + AdipA1 + A8MPO |
| 538 | CAD ~ Intercept.Low + NTproBNP + PlGF |
| 539 | CAD ~ Intercept.Low + AdipA1 + A12TNF |
| 540 | CAD ~ Intercept.Low + NTproBNP + A8MPO |
| 541 | CAD ~ Intercept.Low + NTproBNP + A12TNF |
| 542 | CAD ~ Intercept.Low + PlGF + A8MPO |
| 543 | CAD ~ Intercept.Low + PlGF + A12TNF |
| 544 | CAD ~ Intercept.Low + AdipA1 + NTproBNP + PlGF |
| 545 | CAD ~ Intercept.Low + AdipA1 + NTproBNP + A8MPO |
| 546 | CAD ~ Intercept.Low + AdipA1 + NTproBNP + A12TNF |
| 547 | CAD ~ Intercept.Low + AdipA1 + PlGF + A8MPO |
| 548 | CAD ~ Intercept.Low + AdipA1 + PlGF + A12TNF |
| 549 | CAD ~ Intercept.Low + AdipA1 + A8MPO + A12TNF |
| 550 | CAD ~ Intercept.Low + NTproBNP + PlGF + A8MPO |
| 551 | CAD ~ Intercept.Low + NTproBNP + PlGF + A12TNF |
| 552 | CAD ~ Intercept.Low + NTproBNP + A8MPO + A12TNF |
| 553 | CAD ~ Intercept.Low + PlGF + A8MPO + A12TNF |
| 554 | CAD ~ Intercept.Low + AdipA1 + NTproBNP + PlGF |
| 555 | CAD ~ Intercept.Low + AdipA1 + NTproBNP + A8MPO |
| 556 | CAD ~ Intercept.Low + AdipA1 + NTproBNP + A12TNF |
| 557 | CAD ~ Intercept.Low + AdipA1 + PlGF + A8MPO |
| 558 | CAD ~ Intercept.Low + AdipA1 + PlGF + A12TNF |
| 559 | CAD ~ Intercept.Low + AdipA1 + A8MPO + A12TNF |
| 560 | CAD ~ Intercept.Low + NTproBNP + PlGF + A8MPO |
| 561 | CAD ~ Intercept.Low + NTproBNP + PlGF + A12TNF |
| 562 | CAD ~ Intercept.Low + NTproBNP + A8MPO + A12TNF |
| 563 | CAD ~ Intercept.Low + PlGF + A8MPO + A12TNF |
| 564 | CAD ~ Intercept.Low + AdipA1 + NTproBNP + PlGF + A8MPO |
| 565 | CAD ~ Intercept.Low + AdipA1 + NTproBNP + PlGF + A12TNF |
| 566 | CAD ~ Intercept.Low + AdipA1 + NTproBNP + A8MPO + A12TNF |
| 567 | CAD ~ Intercept.Low + AdipA1 + PlGF + A8MPO + A12TNF |
| 568 | CAD ~ Intercept.Low + NTproBNP + PlGF + A8MPO + A12TNF |
| 569 | CAD ~ Intercept.Low + AdipA1 + NTproBNP + PlGF |
| 570 | CAD ~ Intercept.Low + AdipA1 + NTproBNP + A8MPO |
| 571 | CAD ~ Intercept.Low + AdipA1 + NTproBNP + A12TNF |
| 572 | CAD ~ Intercept.Low + AdipA1 + PlGF + A8MPO |
| 573 | CAD ~ Intercept.Low + AdipA1 + PlGF + A12TNF |
| 574 | CAD ~ Intercept.Low + AdipA1 + A8MPO + A12TNF |
| 575 | CAD ~ Intercept.Low + NTproBNP + PlGF + A8MPO |
| 576 | CAD ~ Intercept.Low + NTproBNP + PlGF + A12TNF |
| 577 | CAD ~ Intercept.Low + NTproBNP + A8MPO + A12TNF |
| 578 | CAD ~ Intercept.Low + PlGF + A8MPO + A12TNF |
| 579 | CAD ~ Intercept.Low + AdipA1 + NTproBNP + PlGF + A8MPO |
| 580 | CAD ~ Intercept.Low + AdipA1 + NTproBNP + PlGF + A12TNF |
| 581 | CAD ~ Intercept.Low + AdipA1 + NTproBNP + A8MPO + A12TNF |
| 582 | CAD ~ Intercept.Low + AdipA1 + PlGF + A8MPO + A12TNF |
| 583 | CAD ~ Intercept.Low + NTproBNP + PlGF + A8MPO + A12TNF |
| 584 | CAD ~ Intercept.Low + AdipA1 + NTproBNP + PlGF + A8MPO |
| 585 | CAD ~ Intercept.Low + AdipA1 + NTproBNP + PlGF + A12TNF |
| 586 | CAD ~ Intercept.Low + AdipA1 + NTproBNP + A8MPO + A12TNF |
| 587 | CAD ~ Intercept.Low + AdipA1 + PlGF + A8MPO + A12TNF |
| 588 | CAD ~ Intercept.Low + NTproBNP + PlGF + A8MPO + A12TNF |
| 589 | CAD ~ Intercept.Low + AdipA1 + NTproBNP + PlGF + A8MPO + A12TNF |
| 590 | CAD ~ Intercept.Low + A8MPO + A12TNF |
| 591 | CAD ~ Intercept.Low + AdipA1 + NTproBNP |
| 592 | CAD ~ Intercept.Low + AdipA1 + PlGF |
| 593 | CAD ~ Intercept.Low + AdipA1 + A8MPO |
| 594 | CAD ~ Intercept.Low + NTproBNP + PlGF |
| 595 | CAD ~ Intercept.Low + AdipA1 + A12TNF |
| 596 | CAD ~ Intercept.Low + NTproBNP + A8MPO |
| 597 | CAD ~ Intercept.Low + NTproBNP + A12TNF |
| 598 | CAD ~ Intercept.Low + PlGF + A8MPO |
| 599 | CAD ~ Intercept.Low + PlGF + A12TNF |
| 600 | CAD ~ Intercept.Low + AdipA1 + NTproBNP + PlGF |
| 601 | CAD ~ Intercept.Low + AdipA1 + NTproBNP + A8MPO |
| 602 | CAD ~ Intercept.Low + AdipA1 + NTproBNP + A12TNF |
| 603 | CAD ~ Intercept.Low + AdipA1 + PlGF + A8MPO |
| 604 | CAD ~ Intercept.Low + AdipA1 + PlGF + A12TNF |
| 605 | CAD ~ Intercept.Low + AdipA1 + A8MPO + A12TNF |
| 606 | CAD ~ Intercept.Low + NTproBNP + PlGF + A8MPO |
| 607 | CAD ~ Intercept.Low + NTproBNP + PlGF + A12TNF |
| 608 | CAD ~ Intercept.Low + NTproBNP + A8MPO + A12TNF |
| 609 | CAD ~ Intercept.Low + PlGF + A8MPO + A12TNF |
| 610 | CAD ~ Intercept.Low + AdipA1 + NTproBNP + PlGF |
| 611 | CAD ~ Intercept.Low + AdipA1 + NTproBNP + A8MPO |
| 612 | CAD ~ Intercept.Low + AdipA1 + NTproBNP + A12TNF |
| 613 | CAD ~ Intercept.Low + AdipA1 + PlGF + A8MPO |
| 614 | CAD ~ Intercept.Low + AdipA1 + PlGF + A12TNF |
| 615 | CAD ~ Intercept.Low + AdipA1 + A8MPO + A12TNF |
| 616 | CAD ~ Intercept.Low + NTproBNP + PlGF + A8MPO |
| 617 | CAD ~ Intercept.Low + NTproBNP + PlGF + A12TNF |
| 618 | CAD ~ Intercept.Low + NTproBNP + A8MPO + A12TNF |
| 619 | CAD ~ Intercept.Low + PlGF + A8MPO + A12TNF |
| 620 | CAD ~ Intercept.Low + AdipA1 + NTproBNP + PlGF + A8MPO |
| 621 | CAD ~ Intercept.Low + AdipA1 + NTproBNP + PlGF + A12TNF |
| 622 | CAD ~ Intercept.Low + AdipA1 + NTproBNP + A8MPO + A12TNF |
| 623 | CAD ~ Intercept.Low + AdipA1 + PlGF + A8MPO + A12TNF |
| 624 | CAD ~ Intercept.Low + NTproBNP + PlGF + A8MPO + A12TNF |
| 625 | CAD ~ Intercept.Low + AdipA1 + NTproBNP + PlGF |
| 626 | CAD ~ Intercept.Low + AdipA1 + NTproBNP + A8MPO |
| 627 | CAD ~ Intercept.Low + AdipA1 + NTproBNP + A12TNF |
| 628 | CAD ~ Intercept.Low + AdipA1 + PlGF + A8MPO |
| 629 | CAD ~ Intercept.Low + AdipA1 + PlGF + A12TNF |
| 630 | CAD ~ Intercept.Low + AdipA1 + A8MPO + A12TNF |
| 631 | CAD ~ Intercept.Low + NTproBNP + PlGF + A8MPO |
| 632 | CAD ~ Intercept.Low + NTproBNP + PlGF + A12TNF |
| 633 | CAD ~ Intercept.Low + NTproBNP + A8MPO + A12TNF |
| 634 | CAD ~ Intercept.Low + PlGF + A8MPO + A12TNF |
| 635 | CAD ~ Intercept.Low + AdipA1 + NTproBNP + PlGF + A8MPO |
| 636 | CAD ~ Intercept.Low + AdipA1 + NTproBNP + PlGF + A12TNF |
| 637 | CAD ~ Intercept.Low + AdipA1 + NTproBNP + A8MPO + A12TNF |
| 638 | CAD ~ Intercept.Low + AdipA1 + PlGF + A8MPO + A12TNF |
| 639 | CAD ~ Intercept.Low + NTproBNP + PlGF + A8MPO + A12TNF |
| 640 | CAD ~ Intercept.Low + AdipA1 + NTproBNP + PlGF + A8MPO |
| 641 | CAD ~ Intercept.Low + AdipA1 + NTproBNP + PlGF + A12TNF |
| 642 | CAD ~ Intercept.Low + AdipA1 + NTproBNP + A8MPO + A12TNF |
| 643 | CAD ~ Intercept.Low + AdipA1 + PlGF + A8MPO + A12TNF |
| 644 | CAD ~ Intercept.Low + NTproBNP + PlGF + A8MPO + A12TNF |
| 645 | CAD ~ Intercept.Low + AdipA1 + NTproBNP + PlGF + A8MPO + A12TNF |
| 646 | CAD ~ Intercept.Low + AdipA1 + NTproBNP + PlGF |
| 647 | CAD ~ Intercept.Low + AdipA1 + NTproBNP + A8MPO |
| 648 | CAD ~ Intercept.Low + AdipA1 + NTproBNP + A12TNF |
| 649 | CAD ~ Intercept.Low + AdipA1 + PlGF + A8MPO |
| 650 | CAD ~ Intercept.Low + AdipA1 + PlGF + A12TNF |
| 651 | CAD ~ Intercept.Low + AdipA1 + A8MPO + A12TNF |
| 652 | CAD ~ Intercept.Low + NTproBNP + PlGF + A8MPO |
| 653 | CAD ~ Intercept.Low + NTproBNP + PlGF + A12TNF |
| 654 | CAD ~ Intercept.Low + NTproBNP + A8MPO + A12TNF |
| 655 | CAD ~ Intercept.Low + PlGF + A8MPO + A12TNF |
| 656 | CAD ~ Intercept.Low + AdipA1 + NTproBNP + PlGF + A8MPO |
| 657 | CAD ~ Intercept.Low + AdipA1 + NTproBNP + PlGF + A12TNF |
| 658 | CAD ~ Intercept.Low + AdipA1 + NTproBNP + A8MPO + A12TNF |
| 659 | CAD ~ Intercept.Low + AdipA1 + PlGF + A8MPO + A12TNF |
| 660 | CAD ~ Intercept.Low + NTproBNP + PlGF + A8MPO + A12TNF |
| 661 | CAD ~ Intercept.Low + AdipA1 + NTproBNP + PlGF + A8MPO |
| 662 | CAD ~ Intercept.Low + AdipA1 + NTproBNP + PlGF + A12TNF |
| 663 | CAD ~ Intercept.Low + AdipA1 + NTproBNP + A8MPO + A12TNF |
| 664 | CAD ~ Intercept.Low + AdipA1 + PlGF + A8MPO + A12TNF |
| 665 | CAD ~ Intercept.Low + NTproBNP + PlGF + A8MPO + A12TNF |
| 666 | CAD ~ Intercept.Low + AdipA1 + NTproBNP + PlGF + A8MPO + A12TNF |
| 667 | CAD ~ Intercept.Low + AdipA1 + NTproBNP + PlGF + A8MPO |
| 668 | CAD ~ Intercept.Low + AdipA1 + NTproBNP + PlGF + A12TNF |
| 669 | CAD ~ Intercept.Low + AdipA1 + NTproBNP + A8MPO + A12TNF |
| 670 | CAD ~ Intercept.Low + AdipA1 + PlGF + A8MPO + A12TNF |
| 671 | CAD ~ Intercept.Low + NTproBNP + PlGF + A8MPO + A12TNF |
| 672 | CAD ~ Intercept.Low + AdipA1 + NTproBNP + PlGF + A8MPO + A12TNF |
| 673 | CAD ~ Intercept.Low + AdipA1 + NTproBNP + PlGF + A8MPO + A12TNF |
| 674 | CAD ~ Intercept.Low + A8MPO + A12TNF |
| 675 | CAD ~ Intercept.Low + AdipA1 + NTproBNP |
| 676 | CAD ~ Intercept.Low + AdipA1 + PlGF |
| 677 | CAD ~ Intercept.Low + AdipA1 + A8MPO |
| 678 | CAD ~ Intercept.Low + AdipA1 + A12TNF |
| 679 | CAD ~ Intercept.Low + NTproBNP + PlGF |
| 680 | CAD ~ Intercept.Low + NTproBNP + A8MPO |
| 681 | CAD ~ Intercept.Low + NTproBNP + A12TNF |
| 682 | CAD ~ Intercept.Low + PlGF + A8MPO |
| 683 | CAD ~ Intercept.Low + PlGF + A12TNF |
| 684 | CAD ~ Intercept.Low + AdipA1 + NTproBNP + PlGF |
| 685 | CAD ~ Intercept.Low + AdipA1 + NTproBNP + A8MPO |
| 686 | CAD ~ Intercept.Low + AdipA1 + NTproBNP + A12TNF |
| 687 | CAD ~ Intercept.Low + AdipA1 + PlGF + A8MPO |
| 688 | CAD ~ Intercept.Low + AdipA1 + PlGF + A12TNF |
| 689 | CAD ~ Intercept.Low + AdipA1 + A8MPO + A12TNF |
| 690 | CAD ~ Intercept.Low + NTproBNP + PlGF + A8MPO |
| 691 | CAD ~ Intercept.Low + NTproBNP + PlGF + A12TNF |
| 692 | CAD ~ Intercept.Low + NTproBNP + A8MPO + A12TNF |
| 693 | CAD ~ Intercept.Low + PlGF + A8MPO + A12TNF |
| 694 | CAD ~ Intercept.Low + AdipA1 + NTproBNP + PlGF |
| 695 | CAD ~ Intercept.Low + AdipA1 + NTproBNP + A8MPO |
| 696 | CAD ~ Intercept.Low + AdipA1 + NTproBNP + A12TNF |
| 697 | CAD ~ Intercept.Low + AdipA1 + PlGF + A8MPO |
| 698 | CAD ~ Intercept.Low + AdipA1 + PlGF + A12TNF |
| 699 | CAD ~ Intercept.Low + AdipA1 + A8MPO + A12TNF |
| 700 | CAD ~ Intercept.Low + NTproBNP + PlGF + A8MPO |
| 701 | CAD ~ Intercept.Low + NTproBNP + PlGF + A12TNF |
| 702 | CAD ~ Intercept.Low + NTproBNP + A8MPO + A12TNF |
| 703 | CAD ~ Intercept.Low + PlGF + A8MPO + A12TNF |
| 704 | CAD ~ Intercept.Low + AdipA1 + NTproBNP + PlGF + A8MPO |
| 705 | CAD ~ Intercept.Low + AdipA1 + NTproBNP + PlGF + A12TNF |
| 706 | CAD ~ Intercept.Low + AdipA1 + NTproBNP + A8MPO + A12TNF |
| 707 | CAD ~ Intercept.Low + AdipA1 + PlGF + A8MPO + A12TNF |
| 708 | CAD ~ Intercept.Low + NTproBNP + PlGF + A8MPO + A12TNF |
| 709 | CAD ~ Intercept.Low + AdipA1 + NTproBNP + PlGF |
| 710 | CAD ~ Intercept.Low + AdipA1 + NTproBNP + A8MPO |
| 711 | CAD ~ Intercept.Low + AdipA1 + NTproBNP + A12TNF |
| 712 | CAD ~ Intercept.Low + AdipA1 + PlGF + A8MPO |
| 713 | CAD ~ Intercept.Low + AdipA1 + PlGF + A12TNF |
| 714 | CAD ~ Intercept.Low + AdipA1 + A8MPO + A12TNF |
| 715 | CAD ~ Intercept.Low + NTproBNP + PlGF + A8MPO |
| 716 | CAD ~ Intercept.Low + NTproBNP + PlGF + A12TNF |
| 717 | CAD ~ Intercept.Low + NTproBNP + A8MPO + A12TNF |
| 718 | CAD ~ Intercept.Low + PlGF + A8MPO + A12TNF |
| 719 | CAD ~ Intercept.Low + AdipA1 + NTproBNP + PlGF + A8MPO |
| 720 | CAD ~ Intercept.Low + AdipA1 + NTproBNP + PlGF + A12TNF |
| 721 | CAD ~ Intercept.Low + AdipA1 + NTproBNP + A8MPO + A12TNF |
| 722 | CAD ~ Intercept.Low + AdipA1 + PlGF + A8MPO + A12TNF |
| 723 | CAD ~ Intercept.Low + NTproBNP + PlGF + A8MPO + A12TNF |
| 724 | CAD ~ Intercept.Low + AdipA1 + NTproBNP + PlGF + A8MPO |
| 725 | CAD ~ Intercept.Low + AdipA1 + NTproBNP + PlGF + A12TNF |
| 726 | CAD ~ Intercept.Low + AdipA1 + NTproBNP + A8MPO + A12TNF |
| 727 | CAD ~ Intercept.Low + AdipA1 + PlGF + A8MPO + A12TNF |
| 728 | CAD ~ Intercept.Low + NTproBNP + PlGF + A8MPO + A12TNF |
| 729 | CAD ~ Intercept.Low + AdipA1 + NTproBNP + PlGF + A8MPO + A12TNF |
| 730 | CAD ~ Intercept.Low + AdipA1 + NTproBNP + PlGF |
| 731 | CAD ~ Intercept.Low + AdipA1 + NTproBNP + A8MPO |
| 732 | CAD ~ Intercept.Low + AdipA1 + NTproBNP + A12TNF |
| 733 | CAD ~ Intercept.Low + AdipA1 + PlGF + A8MPO |
| 734 | CAD ~ Intercept.Low + AdipA1 + PlGF + A12TNF |
| 735 | CAD ~ Intercept.Low + AdipA1 + A8MPO + A12TNF |
| 736 | CAD ~ Intercept.Low + NTproBNP + PlGF + A8MPO |
| 737 | CAD ~ Intercept.Low + NTproBNP + PlGF + A12TNF |
| 738 | CAD ~ Intercept.Low + NTproBNP + A8MPO + A12TNF |
| 739 | CAD ~ Intercept.Low + PlGF + A8MPO + A12TNF |
| 740 | CAD ~ Intercept.Low + AdipA1 + NTproBNP + PlGF + A8MPO |
| 741 | CAD ~ Intercept.Low + AdipA1 + NTproBNP + PlGF + A12TNF |
| 742 | CAD ~ Intercept.Low + AdipA1 + NTproBNP + A8MPO + A12TNF |
| 743 | CAD ~ Intercept.Low + AdipA1 + PlGF + A8MPO + A12TNF |
| 744 | CAD ~ Intercept.Low + NTproBNP + PlGF + A8MPO + A12TNF |
| 745 | CAD ~ Intercept.Low + AdipA1 + NTproBNP + PlGF + A8MPO |
| 746 | CAD ~ Intercept.Low + AdipA1 + NTproBNP + PlGF + A12TNF |
| 747 | CAD ~ Intercept.Low + AdipA1 + NTproBNP + A8MPO + A12TNF |
| 748 | CAD ~ Intercept.Low + AdipA1 + PlGF + A8MPO + A12TNF |
| 749 | CAD ~ Intercept.Low + NTproBNP + PlGF + A8MPO + A12TNF |
| 750 | CAD ~ Intercept.Low + AdipA1 + NTproBNP + PlGF + A8MPO + A12TNF |
| 751 | CAD ~ Intercept.Low + AdipA1 + NTproBNP + PlGF + A8MPO |
| 752 | CAD ~ Intercept.Low + AdipA1 + NTproBNP + PlGF + A12TNF |
| 753 | CAD ~ Intercept.Low + AdipA1 + NTproBNP + A8MPO + A12TNF |
| 754 | CAD ~ Intercept.Low + AdipA1 + PlGF + A8MPO + A12TNF |
| 755 | CAD ~ Intercept.Low + NTproBNP + PlGF + A8MPO + A12TNF |
| 756 | CAD ~ Intercept.Low + AdipA1 + NTproBNP + PlGF + A8MPO + A12TNF |
| 757 | CAD ~ Intercept.Low + AdipA1 + NTproBNP + PlGF + A8MPO + A12TNF |
| 758 | CAD ~ Intercept.Low + AdipA1 + NTproBNP + PlGF |
| 759 | CAD ~ Intercept.Low + AdipA1 + NTproBNP + A8MPO |
| 760 | CAD ~ Intercept.Low + AdipA1 + NTproBNP + A12TNF |
| 761 | CAD ~ Intercept.Low + AdipA1 + PlGF + A8MPO |
| 762 | CAD ~ Intercept.Low + AdipA1 + PlGF + A12TNF |
| 763 | CAD ~ Intercept.Low + AdipA1 + A8MPO + A12TNF |
| 764 | CAD ~ Intercept.Low + NTproBNP + PlGF + A8MPO |
| 765 | CAD ~ Intercept.Low + NTproBNP + PlGF + A12TNF |
| 766 | CAD ~ Intercept.Low + NTproBNP + A8MPO + A12TNF |
| 767 | CAD ~ Intercept.Low + PlGF + A8MPO + A12TNF |
| 768 | CAD ~ Intercept.Low + AdipA1 + NTproBNP + PlGF + A8MPO |
| 769 | CAD ~ Intercept.Low + AdipA1 + NTproBNP + PlGF + A12TNF |
| 770 | CAD ~ Intercept.Low + AdipA1 + NTproBNP + A8MPO + A12TNF |
| 771 | CAD ~ Intercept.Low + AdipA1 + PlGF + A8MPO + A12TNF |
| 772 | CAD ~ Intercept.Low + NTproBNP + PlGF + A8MPO + A12TNF |
| 773 | CAD ~ Intercept.Low + AdipA1 + NTproBNP + PlGF + A8MPO |
| 774 | CAD ~ Intercept.Low + AdipA1 + NTproBNP + PlGF + A12TNF |
| 775 | CAD ~ Intercept.Low + AdipA1 + NTproBNP + A8MPO + A12TNF |
| 776 | CAD ~ Intercept.Low + AdipA1 + PlGF + A8MPO + A12TNF |
| 777 | CAD ~ Intercept.Low + NTproBNP + PlGF + A8MPO + A12TNF |
| 778 | CAD ~ Intercept.Low + AdipA1 + NTproBNP + PlGF + A8MPO + A12TNF |
| 779 | CAD ~ Intercept.Low + AdipA1 + NTproBNP + PlGF + A8MPO |
| 780 | CAD ~ Intercept.Low + AdipA1 + NTproBNP + PlGF + A12TNF |
| 781 | CAD ~ Intercept.Low + AdipA1 + NTproBNP + A8MPO + A12TNF |
| 782 | CAD ~ Intercept.Low + AdipA1 + PlGF + A8MPO + A12TNF |
| 783 | CAD ~ Intercept.Low + NTproBNP + PlGF + A8MPO + A12TNF |
| 784 | CAD ~ Intercept.Low + AdipA1 + NTproBNP + PlGF + A8MPO + A12TNF |
| 785 | CAD ~ Intercept.Low + AdipA1 + NTproBNP + PlGF + A8MPO + A12TNF |
| 786 | CAD ~ Intercept.Low + AdipA1 + NTproBNP + PlGF + A8MPO |
| 787 | CAD ~ Intercept.Low + AdipA1 + NTproBNP + PlGF + A12TNF |
| 788 | CAD ~ Intercept.Low + AdipA1 + NTproBNP + A8MPO + A12TNF |
| 789 | CAD ~ Intercept.Low + AdipA1 + PlGF + A8MPO + A12TNF |
| 790 | CAD ~ Intercept.Low + NTproBNP + PlGF + A8MPO + A12TNF |
| 791 | CAD ~ Intercept.Low + AdipA1 + NTproBNP + PlGF + A8MPO + A12TNF |
| 792 | CAD ~ Intercept.Low + AdipA1 + NTproBNP + PlGF + A8MPO + A12TNF |
| 793 | CAD ~ Intercept.Low + AdipA1 + NTproBNP + PlGF + A8MPO + A12TNF |
| 794 | CAD ~ Intercept.Low + AdipA1 + NTproBNP + PlGF + A8MPO + A12TNF |
| 795 | CAD ~ Intercept.Low + NTproBNP |
| 796 | CAD ~ Intercept.Low + AdipA1 |
| 797 | CAD ~ Intercept.Low + PlGF |
| 798 | CAD ~ Intercept.Low + A8MPO |
| 799 | CAD ~ Intercept.Low + A12TNF |
| 800 | CAD ~ Intercept.Low + A8MPO + A12TNF |
| 801 | CAD ~ Intercept.Low + AdipA1 + NTproBNP |
| 802 | CAD ~ Intercept.Low + AdipA1 + PlGF |
| 803 | CAD ~ Intercept.Low + AdipA1 + A8MPO |
| 804 | CAD ~ Intercept.Low + NTproBNP + PlGF |
| 805 | CAD ~ Intercept.Low + AdipA1 + A12TNF |
| 806 | CAD ~ Intercept.Low + NTproBNP + A8MPO |
| 807 | CAD ~ Intercept.Low + NTproBNP + A12TNF |
| 808 | CAD ~ Intercept.Low + PlGF + A8MPO |
| 809 | CAD ~ Intercept.Low + PlGF + A12TNF |
| 810 | CAD ~ Intercept.Low + A8MPO + A12TNF |
| 811 | CAD ~ Intercept.Low + AdipA1 + NTproBNP |
| 812 | CAD ~ Intercept.Low + AdipA1 + PlGF |
| 813 | CAD ~ Intercept.Low + AdipA1 + A8MPO |
| 814 | CAD ~ Intercept.Low + NTproBNP + PlGF |
| 815 | CAD ~ Intercept.Low + AdipA1 + A12TNF |
| 816 | CAD ~ Intercept.Low + NTproBNP + A8MPO |
| 817 | CAD ~ Intercept.Low + NTproBNP + A12TNF |
| 818 | CAD ~ Intercept.Low + PlGF + A8MPO |
| 819 | CAD ~ Intercept.Low + PlGF + A12TNF |
| 820 | CAD ~ Intercept.Low + AdipA1 + NTproBNP + PlGF |
| 821 | CAD ~ Intercept.Low + AdipA1 + NTproBNP + A8MPO |
| 822 | CAD ~ Intercept.Low + AdipA1 + NTproBNP + A12TNF |
| 823 | CAD ~ Intercept.Low + AdipA1 + PlGF + A8MPO |
| 824 | CAD ~ Intercept.Low + AdipA1 + PlGF + A12TNF |
| 825 | CAD ~ Intercept.Low + AdipA1 + A8MPO + A12TNF |
| 826 | CAD ~ Intercept.Low + NTproBNP + PlGF + A8MPO |
| 827 | CAD ~ Intercept.Low + NTproBNP + PlGF + A12TNF |
| 828 | CAD ~ Intercept.Low + NTproBNP + A8MPO + A12TNF |
| 829 | CAD ~ Intercept.Low + PlGF + A8MPO + A12TNF |
| 830 | CAD ~ Intercept.Low + A8MPO + A12TNF |
| 831 | CAD ~ Intercept.Low + AdipA1 + NTproBNP |
| 832 | CAD ~ Intercept.Low + AdipA1 + PlGF |
| 833 | CAD ~ Intercept.Low + AdipA1 + A8MPO |
| 834 | CAD ~ Intercept.Low + NTproBNP + PlGF |
| 835 | CAD ~ Intercept.Low + AdipA1 + A12TNF |
| 836 | CAD ~ Intercept.Low + NTproBNP + A8MPO |
| 837 | CAD ~ Intercept.Low + NTproBNP + A12TNF |
| 838 | CAD ~ Intercept.Low + PlGF + A8MPO |
| 839 | CAD ~ Intercept.Low + PlGF + A12TNF |
| 840 | CAD ~ Intercept.Low + AdipA1 + NTproBNP + PlGF |
| 841 | CAD ~ Intercept.Low + AdipA1 + NTproBNP + A8MPO |
| 842 | CAD ~ Intercept.Low + AdipA1 + NTproBNP + A12TNF |
| 843 | CAD ~ Intercept.Low + AdipA1 + PlGF + A8MPO |
| 844 | CAD ~ Intercept.Low + AdipA1 + PlGF + A12TNF |
| 845 | CAD ~ Intercept.Low + AdipA1 + A8MPO + A12TNF |
| 846 | CAD ~ Intercept.Low + NTproBNP + PlGF + A8MPO |
| 847 | CAD ~ Intercept.Low + NTproBNP + PlGF + A12TNF |
| 848 | CAD ~ Intercept.Low + NTproBNP + A8MPO + A12TNF |
| 849 | CAD ~ Intercept.Low + PlGF + A8MPO + A12TNF |
| 850 | CAD ~ Intercept.Low + AdipA1 + NTproBNP + PlGF |
| 851 | CAD ~ Intercept.Low + AdipA1 + NTproBNP + A8MPO |
| 852 | CAD ~ Intercept.Low + AdipA1 + NTproBNP + A12TNF |
| 853 | CAD ~ Intercept.Low + AdipA1 + PlGF + A8MPO |
| 854 | CAD ~ Intercept.Low + AdipA1 + PlGF + A12TNF |
| 855 | CAD ~ Intercept.Low + AdipA1 + A8MPO + A12TNF |
| 856 | CAD ~ Intercept.Low + NTproBNP + PlGF + A8MPO |
| 857 | CAD ~ Intercept.Low + NTproBNP + PlGF + A12TNF |
| 858 | CAD ~ Intercept.Low + NTproBNP + A8MPO + A12TNF |
| 859 | CAD ~ Intercept.Low + PlGF + A8MPO + A12TNF |
| 860 | CAD ~ Intercept.Low + AdipA1 + NTproBNP + PlGF + A8MPO |
| 861 | CAD ~ Intercept.Low + AdipA1 + NTproBNP + PlGF + A12TNF |
| 862 | CAD ~ Intercept.Low + AdipA1 + NTproBNP + A8MPO + A12TNF |
| 863 | CAD ~ Intercept.Low + AdipA1 + PlGF + A8MPO + A12TNF |
| 864 | CAD ~ Intercept.Low + NTproBNP + PlGF + A8MPO + A12TNF |
| 865 | CAD ~ Intercept.Low + A8MPO + A12TNF |
| 866 | CAD ~ Intercept.Low + AdipA1 + NTproBNP |
| 867 | CAD ~ Intercept.Low + AdipA1 + PlGF |
| 868 | CAD ~ Intercept.Low + AdipA1 + A8MPO |
| 869 | CAD ~ Intercept.Low + NTproBNP + PlGF |
| 870 | CAD ~ Intercept.Low + AdipA1 + A12TNF |
| 871 | CAD ~ Intercept.Low + NTproBNP + A8MPO |
| 872 | CAD ~ Intercept.Low + NTproBNP + A12TNF |
| 873 | CAD ~ Intercept.Low + PlGF + A8MPO |
| 874 | CAD ~ Intercept.Low + PlGF + A12TNF |
| 875 | CAD ~ Intercept.Low + AdipA1 + NTproBNP + PlGF |
| 876 | CAD ~ Intercept.Low + AdipA1 + NTproBNP + A8MPO |
| 877 | CAD ~ Intercept.Low + AdipA1 + NTproBNP + A12TNF |
| 878 | CAD ~ Intercept.Low + AdipA1 + PlGF + A8MPO |
| 879 | CAD ~ Intercept.Low + AdipA1 + PlGF + A12TNF |
| 880 | CAD ~ Intercept.Low + AdipA1 + A8MPO + A12TNF |
| 881 | CAD ~ Intercept.Low + NTproBNP + PlGF + A8MPO |
| 882 | CAD ~ Intercept.Low + NTproBNP + PlGF + A12TNF |
| 883 | CAD ~ Intercept.Low + NTproBNP + A8MPO + A12TNF |
| 884 | CAD ~ Intercept.Low + PlGF + A8MPO + A12TNF |
| 885 | CAD ~ Intercept.Low + AdipA1 + NTproBNP + PlGF |
| 886 | CAD ~ Intercept.Low + AdipA1 + NTproBNP + A8MPO |
| 887 | CAD ~ Intercept.Low + AdipA1 + NTproBNP + A12TNF |
| 888 | CAD ~ Intercept.Low + AdipA1 + PlGF + A8MPO |
| 889 | CAD ~ Intercept.Low + AdipA1 + PlGF + A12TNF |
| 890 | CAD ~ Intercept.Low + AdipA1 + A8MPO + A12TNF |
| 891 | CAD ~ Intercept.Low + NTproBNP + PlGF + A8MPO |
| 892 | CAD ~ Intercept.Low + NTproBNP + PlGF + A12TNF |
| 893 | CAD ~ Intercept.Low + NTproBNP + A8MPO + A12TNF |
| 894 | CAD ~ Intercept.Low + PlGF + A8MPO + A12TNF |
| 895 | CAD ~ Intercept.Low + AdipA1 + NTproBNP + PlGF + A8MPO |
| 896 | CAD ~ Intercept.Low + AdipA1 + NTproBNP + PlGF + A12TNF |
| 897 | CAD ~ Intercept.Low + AdipA1 + NTproBNP + A8MPO + A12TNF |
| 898 | CAD ~ Intercept.Low + AdipA1 + PlGF + A8MPO + A12TNF |
| 899 | CAD ~ Intercept.Low + NTproBNP + PlGF + A8MPO + A12TNF |
| 900 | CAD ~ Intercept.Low + AdipA1 + NTproBNP + PlGF |
| 901 | CAD ~ Intercept.Low + AdipA1 + NTproBNP + A8MPO |
| 902 | CAD ~ Intercept.Low + AdipA1 + NTproBNP + A12TNF |
| 903 | CAD ~ Intercept.Low + AdipA1 + PlGF + A8MPO |
| 904 | CAD ~ Intercept.Low + AdipA1 + PlGF + A12TNF |
| 905 | CAD ~ Intercept.Low + AdipA1 + A8MPO + A12TNF |
| 906 | CAD ~ Intercept.Low + NTproBNP + PlGF + A8MPO |
| 907 | CAD ~ Intercept.Low + NTproBNP + PlGF + A12TNF |
| 908 | CAD ~ Intercept.Low + NTproBNP + A8MPO + A12TNF |
| 909 | CAD ~ Intercept.Low + PlGF + A8MPO + A12TNF |
| 910 | CAD ~ Intercept.Low + AdipA1 + NTproBNP + PlGF + A8MPO |
| 911 | CAD ~ Intercept.Low + AdipA1 + NTproBNP + PlGF + A12TNF |
| 912 | CAD ~ Intercept.Low + AdipA1 + NTproBNP + A8MPO + A12TNF |
| 913 | CAD ~ Intercept.Low + AdipA1 + PlGF + A8MPO + A12TNF |
| 914 | CAD ~ Intercept.Low + NTproBNP + PlGF + A8MPO + A12TNF |
| 915 | CAD ~ Intercept.Low + AdipA1 + NTproBNP + PlGF + A8MPO |
| 916 | CAD ~ Intercept.Low + AdipA1 + NTproBNP + PlGF + A12TNF |
| 917 | CAD ~ Intercept.Low + AdipA1 + NTproBNP + A8MPO + A12TNF |
| 918 | CAD ~ Intercept.Low + AdipA1 + PlGF + A8MPO + A12TNF |
| 919 | CAD ~ Intercept.Low + NTproBNP + PlGF + A8MPO + A12TNF |
| 920 | CAD ~ Intercept.Low + AdipA1 + NTproBNP + PlGF + A8MPO + A12TNF |
| 921 | CAD ~ Intercept.Low + A8MPO + A12TNF |
| 922 | CAD ~ Intercept.Low + AdipA1 + NTproBNP |
| 923 | CAD ~ Intercept.Low + AdipA1 + PlGF |
| 924 | CAD ~ Intercept.Low + AdipA1 + A8MPO |
| 925 | CAD ~ Intercept.Low + NTproBNP + PlGF |
| 926 | CAD ~ Intercept.Low + AdipA1 + A12TNF |
| 927 | CAD ~ Intercept.Low + NTproBNP + A8MPO |
| 928 | CAD ~ Intercept.Low + NTproBNP + A12TNF |
| 929 | CAD ~ Intercept.Low + PlGF + A8MPO |
| 930 | CAD ~ Intercept.Low + PlGF + A12TNF |
| 931 | CAD ~ Intercept.Low + AdipA1 + NTproBNP + PlGF |
| 932 | CAD ~ Intercept.Low + AdipA1 + NTproBNP + A8MPO |
| 933 | CAD ~ Intercept.Low + AdipA1 + NTproBNP + A12TNF |
| 934 | CAD ~ Intercept.Low + AdipA1 + PlGF + A8MPO |
| 935 | CAD ~ Intercept.Low + AdipA1 + PlGF + A12TNF |
| 936 | CAD ~ Intercept.Low + AdipA1 + A8MPO + A12TNF |
| 937 | CAD ~ Intercept.Low + NTproBNP + PlGF + A8MPO |
| 938 | CAD ~ Intercept.Low + NTproBNP + PlGF + A12TNF |
| 939 | CAD ~ Intercept.Low + NTproBNP + A8MPO + A12TNF |
| 940 | CAD ~ Intercept.Low + PlGF + A8MPO + A12TNF |
| 941 | CAD ~ Intercept.Low + AdipA1 + NTproBNP + PlGF |
| 942 | CAD ~ Intercept.Low + AdipA1 + NTproBNP + A8MPO |
| 943 | CAD ~ Intercept.Low + AdipA1 + NTproBNP + A12TNF |
| 944 | CAD ~ Intercept.Low + AdipA1 + PlGF + A8MPO |
| 945 | CAD ~ Intercept.Low + AdipA1 + PlGF + A12TNF |
| 946 | CAD ~ Intercept.Low + AdipA1 + A8MPO + A12TNF |
| 947 | CAD ~ Intercept.Low + NTproBNP + PlGF + A8MPO |
| 948 | CAD ~ Intercept.Low + NTproBNP + PlGF + A12TNF |
| 949 | CAD ~ Intercept.Low + NTproBNP + A8MPO + A12TNF |
| 950 | CAD ~ Intercept.Low + PlGF + A8MPO + A12TNF |
| 951 | CAD ~ Intercept.Low + AdipA1 + NTproBNP + PlGF + A8MPO |
| 952 | CAD ~ Intercept.Low + AdipA1 + NTproBNP + PlGF + A12TNF |
| 953 | CAD ~ Intercept.Low + AdipA1 + NTproBNP + A8MPO + A12TNF |
| 954 | CAD ~ Intercept.Low + AdipA1 + PlGF + A8MPO + A12TNF |
| 955 | CAD ~ Intercept.Low + NTproBNP + PlGF + A8MPO + A12TNF |
| 956 | CAD ~ Intercept.Low + AdipA1 + NTproBNP + PlGF |
| 957 | CAD ~ Intercept.Low + AdipA1 + NTproBNP + A8MPO |
| 958 | CAD ~ Intercept.Low + AdipA1 + NTproBNP + A12TNF |
| 959 | CAD ~ Intercept.Low + AdipA1 + PlGF + A8MPO |
| 960 | CAD ~ Intercept.Low + AdipA1 + PlGF + A12TNF |
| 961 | CAD ~ Intercept.Low + AdipA1 + A8MPO + A12TNF |
| 962 | CAD ~ Intercept.Low + NTproBNP + PlGF + A8MPO |
| 963 | CAD ~ Intercept.Low + NTproBNP + PlGF + A12TNF |
| 964 | CAD ~ Intercept.Low + NTproBNP + A8MPO + A12TNF |
| 965 | CAD ~ Intercept.Low + PlGF + A8MPO + A12TNF |
| 966 | CAD ~ Intercept.Low + AdipA1 + NTproBNP + PlGF + A8MPO |
| 967 | CAD ~ Intercept.Low + AdipA1 + NTproBNP + PlGF + A12TNF |
| 968 | CAD ~ Intercept.Low + AdipA1 + NTproBNP + A8MPO + A12TNF |
| 969 | CAD ~ Intercept.Low + AdipA1 + PlGF + A8MPO + A12TNF |
| 970 | CAD ~ Intercept.Low + NTproBNP + PlGF + A8MPO + A12TNF |
| 971 | CAD ~ Intercept.Low + AdipA1 + NTproBNP + PlGF + A8MPO |
| 972 | CAD ~ Intercept.Low + AdipA1 + NTproBNP + PlGF + A12TNF |
| 973 | CAD ~ Intercept.Low + AdipA1 + NTproBNP + A8MPO + A12TNF |
| 974 | CAD ~ Intercept.Low + AdipA1 + PlGF + A8MPO + A12TNF |
| 975 | CAD ~ Intercept.Low + NTproBNP + PlGF + A8MPO + A12TNF |
| 976 | CAD ~ Intercept.Low + AdipA1 + NTproBNP + PlGF + A8MPO + A12TNF |
| 977 | CAD ~ Intercept.Low + AdipA1 + NTproBNP + PlGF |
| 978 | CAD ~ Intercept.Low + AdipA1 + NTproBNP + A8MPO |
| 979 | CAD ~ Intercept.Low + AdipA1 + NTproBNP + A12TNF |
| 980 | CAD ~ Intercept.Low + AdipA1 + PlGF + A8MPO |
| 981 | CAD ~ Intercept.Low + AdipA1 + PlGF + A12TNF |
| 982 | CAD ~ Intercept.Low + AdipA1 + A8MPO + A12TNF |
| 983 | CAD ~ Intercept.Low + NTproBNP + PlGF + A8MPO |
| 984 | CAD ~ Intercept.Low + NTproBNP + PlGF + A12TNF |
| 985 | CAD ~ Intercept.Low + NTproBNP + A8MPO + A12TNF |
| 986 | CAD ~ Intercept.Low + PlGF + A8MPO + A12TNF |
| 987 | CAD ~ Intercept.Low + AdipA1 + NTproBNP + PlGF + A8MPO |
| 988 | CAD ~ Intercept.Low + AdipA1 + NTproBNP + PlGF + A12TNF |
| 989 | CAD ~ Intercept.Low + AdipA1 + NTproBNP + A8MPO + A12TNF |
| 990 | CAD ~ Intercept.Low + AdipA1 + PlGF + A8MPO + A12TNF |
| 991 | CAD ~ Intercept.Low + NTproBNP + PlGF + A8MPO + A12TNF |
| 992 | CAD ~ Intercept.Low + AdipA1 + NTproBNP + PlGF + A8MPO |
| 993 | CAD ~ Intercept.Low + AdipA1 + NTproBNP + PlGF + A12TNF |
| 994 | CAD ~ Intercept.Low + AdipA1 + NTproBNP + A8MPO + A12TNF |
| 995 | CAD ~ Intercept.Low + AdipA1 + PlGF + A8MPO + A12TNF |
| 996 | CAD ~ Intercept.Low + NTproBNP + PlGF + A8MPO + A12TNF |
| 997 | CAD ~ Intercept.Low + AdipA1 + NTproBNP + PlGF + A8MPO + A12TNF |
| 998 | CAD ~ Intercept.Low + AdipA1 + NTproBNP + PlGF + A8MPO |
| 999 | CAD ~ Intercept.Low + AdipA1 + NTproBNP + PlGF + A12TNF |
| 1000 | CAD ~ Intercept.Low + AdipA1 + NTproBNP + A8MPO + A12TNF |
| 1001 | CAD ~ Intercept.Low + AdipA1 + PlGF + A8MPO + A12TNF |
| 1002 | CAD ~ Intercept.Low + NTproBNP + PlGF + A8MPO + A12TNF |
| 1003 | CAD ~ Intercept.Low + AdipA1 + NTproBNP + PlGF + A8MPO + A12TNF |
| 1004 | CAD ~ Intercept.Low + AdipA1 + NTproBNP + PlGF + A8MPO + A12TNF |
| 1005 | CAD ~ Intercept.Low + A8MPO + A12TNF |
| 1006 | CAD ~ Intercept.Low + AdipA1 + NTproBNP |
| 1007 | CAD ~ Intercept.Low + AdipA1 + PlGF |
| 1008 | CAD ~ Intercept.Low + AdipA1 + A8MPO |
| 1009 | CAD ~ Intercept.Low + NTproBNP + PlGF |
| 1010 | CAD ~ Intercept.Low + AdipA1 + A12TNF |
| 1011 | CAD ~ Intercept.Low + NTproBNP + A8MPO |
| 1012 | CAD ~ Intercept.Low + NTproBNP + A12TNF |
| 1013 | CAD ~ Intercept.Low + PlGF + A8MPO |
| 1014 | CAD ~ Intercept.Low + PlGF + A12TNF |
| 1015 | CAD ~ Intercept.Low + AdipA1 + NTproBNP + PlGF |
| 1016 | CAD ~ Intercept.Low + AdipA1 + NTproBNP + A8MPO |
| 1017 | CAD ~ Intercept.Low + AdipA1 + NTproBNP + A12TNF |
| 1018 | CAD ~ Intercept.Low + AdipA1 + PlGF + A8MPO |
| 1019 | CAD ~ Intercept.Low + AdipA1 + PlGF + A12TNF |
| 1020 | CAD ~ Intercept.Low + AdipA1 + A8MPO + A12TNF |
| 1021 | CAD ~ Intercept.Low + NTproBNP + PlGF + A8MPO |
| 1022 | CAD ~ Intercept.Low + NTproBNP + PlGF + A12TNF |
| 1023 | CAD ~ Intercept.Low + NTproBNP + A8MPO + A12TNF |
| 1024 | CAD ~ Intercept.Low + PlGF + A8MPO + A12TNF |
| 1025 | CAD ~ Intercept.Low + AdipA1 + NTproBNP + PlGF |
| 1026 | CAD ~ Intercept.Low + AdipA1 + NTproBNP + A8MPO |
| 1027 | CAD ~ Intercept.Low + AdipA1 + NTproBNP + A12TNF |
| 1028 | CAD ~ Intercept.Low + AdipA1 + PlGF + A8MPO |
| 1029 | CAD ~ Intercept.Low + AdipA1 + PlGF + A12TNF |
| 1030 | CAD ~ Intercept.Low + AdipA1 + A8MPO + A12TNF |
| 1031 | CAD ~ Intercept.Low + NTproBNP + PlGF + A8MPO |
| 1032 | CAD ~ Intercept.Low + NTproBNP + PlGF + A12TNF |
| 1033 | CAD ~ Intercept.Low + NTproBNP + A8MPO + A12TNF |
| 1034 | CAD ~ Intercept.Low + PlGF + A8MPO + A12TNF |
| 1035 | CAD ~ Intercept.Low + AdipA1 + NTproBNP + PlGF + A8MPO |
| 1036 | CAD ~ Intercept.Low + AdipA1 + NTproBNP + PlGF + A12TNF |
| 1037 | CAD ~ Intercept.Low + AdipA1 + NTproBNP + A8MPO + A12TNF |
| 1038 | CAD ~ Intercept.Low + AdipA1 + PlGF + A8MPO + A12TNF |
| 1039 | CAD ~ Intercept.Low + NTproBNP + PlGF + A8MPO + A12TNF |
| 1040 | CAD ~ Intercept.Low + AdipA1 + NTproBNP + PlGF |
| 1041 | CAD ~ Intercept.Low + AdipA1 + NTproBNP + A8MPO |
| 1042 | CAD ~ Intercept.Low + AdipA1 + NTproBNP + A12TNF |
| 1043 | CAD ~ Intercept.Low + AdipA1 + PlGF + A8MPO |
| 1044 | CAD ~ Intercept.Low + AdipA1 + PlGF + A12TNF |
| 1045 | CAD ~ Intercept.Low + AdipA1 + A8MPO + A12TNF |
| 1046 | CAD ~ Intercept.Low + NTproBNP + PlGF + A8MPO |
| 1047 | CAD ~ Intercept.Low + NTproBNP + PlGF + A12TNF |
| 1048 | CAD ~ Intercept.Low + NTproBNP + A8MPO + A12TNF |
| 1049 | CAD ~ Intercept.Low + PlGF + A8MPO + A12TNF |
| 1050 | CAD ~ Intercept.Low + AdipA1 + NTproBNP + PlGF + A8MPO |
| 1051 | CAD ~ Intercept.Low + AdipA1 + NTproBNP + PlGF + A12TNF |
| 1052 | CAD ~ Intercept.Low + AdipA1 + NTproBNP + A8MPO + A12TNF |
| 1053 | CAD ~ Intercept.Low + AdipA1 + PlGF + A8MPO + A12TNF |
| 1054 | CAD ~ Intercept.Low + NTproBNP + PlGF + A8MPO + A12TNF |
| 1055 | CAD ~ Intercept.Low + AdipA1 + NTproBNP + PlGF + A8MPO |
| 1056 | CAD ~ Intercept.Low + AdipA1 + NTproBNP + PlGF + A12TNF |
| 1057 | CAD ~ Intercept.Low + AdipA1 + NTproBNP + A8MPO + A12TNF |
| 1058 | CAD ~ Intercept.Low + AdipA1 + PlGF + A8MPO + A12TNF |
| 1059 | CAD ~ Intercept.Low + NTproBNP + PlGF + A8MPO + A12TNF |
| 1060 | CAD ~ Intercept.Low + AdipA1 + NTproBNP + PlGF + A8MPO + A12TNF |
| 1061 | CAD ~ Intercept.Low + AdipA1 + NTproBNP + PlGF |
| 1062 | CAD ~ Intercept.Low + AdipA1 + NTproBNP + A8MPO |
| 1063 | CAD ~ Intercept.Low + AdipA1 + NTproBNP + A12TNF |
| 1064 | CAD ~ Intercept.Low + AdipA1 + PlGF + A8MPO |
| 1065 | CAD ~ Intercept.Low + AdipA1 + PlGF + A12TNF |
| 1066 | CAD ~ Intercept.Low + AdipA1 + A8MPO + A12TNF |
| 1067 | CAD ~ Intercept.Low + NTproBNP + PlGF + A8MPO |
| 1068 | CAD ~ Intercept.Low + NTproBNP + PlGF + A12TNF |
| 1069 | CAD ~ Intercept.Low + NTproBNP + A8MPO + A12TNF |
| 1070 | CAD ~ Intercept.Low + PlGF + A8MPO + A12TNF |
| 1071 | CAD ~ Intercept.Low + AdipA1 + NTproBNP + PlGF + A8MPO |
| 1072 | CAD ~ Intercept.Low + AdipA1 + NTproBNP + PlGF + A12TNF |
| 1073 | CAD ~ Intercept.Low + AdipA1 + NTproBNP + A8MPO + A12TNF |
| 1074 | CAD ~ Intercept.Low + AdipA1 + PlGF + A8MPO + A12TNF |
| 1075 | CAD ~ Intercept.Low + NTproBNP + PlGF + A8MPO + A12TNF |
| 1076 | CAD ~ Intercept.Low + AdipA1 + NTproBNP + PlGF + A8MPO |
| 1077 | CAD ~ Intercept.Low + AdipA1 + NTproBNP + PlGF + A12TNF |
| 1078 | CAD ~ Intercept.Low + AdipA1 + NTproBNP + A8MPO + A12TNF |
| 1079 | CAD ~ Intercept.Low + AdipA1 + PlGF + A8MPO + A12TNF |
| 1080 | CAD ~ Intercept.Low + NTproBNP + PlGF + A8MPO + A12TNF |
| 1081 | CAD ~ Intercept.Low + AdipA1 + NTproBNP + PlGF + A8MPO + A12TNF |
| 1082 | CAD ~ Intercept.Low + AdipA1 + NTproBNP + PlGF + A8MPO |
| 1083 | CAD ~ Intercept.Low + AdipA1 + NTproBNP + PlGF + A12TNF |
| 1084 | CAD ~ Intercept.Low + AdipA1 + NTproBNP + A8MPO + A12TNF |
| 1085 | CAD ~ Intercept.Low + AdipA1 + PlGF + A8MPO + A12TNF |
| 1086 | CAD ~ Intercept.Low + NTproBNP + PlGF + A8MPO + A12TNF |
| 1087 | CAD ~ Intercept.Low + AdipA1 + NTproBNP + PlGF + A8MPO + A12TNF |
| 1088 | CAD ~ Intercept.Low + AdipA1 + NTproBNP + PlGF + A8MPO + A12TNF |
| 1089 | CAD ~ Intercept.Low + AdipA1 + NTproBNP + PlGF |
| 1090 | CAD ~ Intercept.Low + AdipA1 + NTproBNP + A8MPO |
| 1091 | CAD ~ Intercept.Low + AdipA1 + NTproBNP + A12TNF |
| 1092 | CAD ~ Intercept.Low + AdipA1 + PlGF + A8MPO |
| 1093 | CAD ~ Intercept.Low + AdipA1 + PlGF + A12TNF |
| 1094 | CAD ~ Intercept.Low + AdipA1 + A8MPO + A12TNF |
| 1095 | CAD ~ Intercept.Low + NTproBNP + PlGF + A8MPO |
| 1096 | CAD ~ Intercept.Low + NTproBNP + PlGF + A12TNF |
| 1097 | CAD ~ Intercept.Low + NTproBNP + A8MPO + A12TNF |
| 1098 | CAD ~ Intercept.Low + PlGF + A8MPO + A12TNF |
| 1099 | CAD ~ Intercept.Low + AdipA1 + NTproBNP + PlGF + A8MPO |
| 1100 | CAD ~ Intercept.Low + AdipA1 + NTproBNP + PlGF + A12TNF |
| 1101 | CAD ~ Intercept.Low + AdipA1 + NTproBNP + A8MPO + A12TNF |
| 1102 | CAD ~ Intercept.Low + AdipA1 + PlGF + A8MPO + A12TNF |
| 1103 | CAD ~ Intercept.Low + NTproBNP + PlGF + A8MPO + A12TNF |
| 1104 | CAD ~ Intercept.Low + AdipA1 + NTproBNP + PlGF + A8MPO |
| 1105 | CAD ~ Intercept.Low + AdipA1 + NTproBNP + PlGF + A12TNF |
| 1106 | CAD ~ Intercept.Low + AdipA1 + NTproBNP + A8MPO + A12TNF |
| 1107 | CAD ~ Intercept.Low + AdipA1 + PlGF + A8MPO + A12TNF |
| 1108 | CAD ~ Intercept.Low + NTproBNP + PlGF + A8MPO + A12TNF |
| 1109 | CAD ~ Intercept.Low + AdipA1 + NTproBNP + PlGF + A8MPO + A12TNF |
| 1110 | CAD ~ Intercept.Low + AdipA1 + NTproBNP + PlGF + A8MPO |
| 1111 | CAD ~ Intercept.Low + AdipA1 + NTproBNP + PlGF + A12TNF |
| 1112 | CAD ~ Intercept.Low + AdipA1 + NTproBNP + A8MPO + A12TNF |
| 1113 | CAD ~ Intercept.Low + AdipA1 + PlGF + A8MPO + A12TNF |
| 1114 | CAD ~ Intercept.Low + NTproBNP + PlGF + A8MPO + A12TNF |
| 1115 | CAD ~ Intercept.Low + AdipA1 + NTproBNP + PlGF + A8MPO + A12TNF |
| 1116 | CAD ~ Intercept.Low + AdipA1 + NTproBNP + PlGF + A8MIPO + A12TNF |
| 1117 | CAD ~ Intercept.Low + NTproBNP |
| 1118 | CAD ~ Intercept.Low + AdipA1 |
| 1119 | CAD ~ Intercept.Low + PlGF |
| 1120 | CAD ~ Intercept.Low + A8MPO |
| 1121 | CAD ~ Intercept.Low + A12TNF |
| 1122 | CAD ~ Intercept.Low + NTproBNP |
| 1123 | CAD ~ Intercept.Low + AdipA1 |
| 1124 | CAD ~ Intercept.Low + PlGF |
| 1125 | CAD ~ Intercept.Low + A8MPO |
| 1126 | CAD ~ Intercept.Low + A12TNF |
| 1127 | CAD ~ Intercept.Low + A8MPO + A12TNF |
| 1128 | CAD ~ Intercept.Low + AdipA1 + NTproBNP |
| 1129 | CAD ~ Intercept.Low + AdipA1 + PlGF |
| 1130 | CAD ~ Intercept.Low + NTproBNP + PlGF |
| 1131 | CAD ~ Intercept.Low + AdipA1 + A8MPO |
| 1132 | CAD ~ Intercept.Low + AdipA1 + A12TNF |
| 1133 | CAD ~ Intercept.Low + NTproBNP + A8MPO |
| 1134 | CAD ~ Intercept.Low + NTproBNP + A12TNF |
| 1135 | CAD ~ Intercept.Low + PlGF + A8MPO |
| 1136 | CAD ~ Intercept.Low + PlGF + A12TNF |
| 1137 | CAD ~ Intercept.Low + NTproBNP |
| 1138 | CAD ~ Intercept.Low + AdipA1 |
| 1139 | CAD ~ Intercept.Low + PlGF |
| 1140 | CAD ~ Intercept.Low + A8MPO |
| 1141 | CAD ~ Intercept.Low + A12TNF |
| 1142 | CAD ~ Intercept.Low + A8MPO + A12TNF |
| 1143 | CAD ~ Intercept.Low + AdipA1 + NTproBNP |
| 1144 | CAD ~ Intercept.Low + AdipA1 + PlGF |
| 1145 | CAD ~ Intercept.Low + NTproBNP + PlGF |
| 1146 | CAD ~ Intercept.Low + AdipA1 + A8MPO |
| 1147 | CAD ~ Intercept.Low + AdipA1 + A12TNF |
| 1148 | CAD ~ Intercept.Low + NTproBNP + A8MPO |
| 1149 | CAD ~ Intercept.Low + NTproBNP + A12TNF |
| 1150 | CAD ~ Intercept.Low + PlGF + A8MPO |
| 1151 | CAD ~ Intercept.Low + PlGF + A12TNF |
| 1152 | CAD ~ Intercept.Low + A8MPO + A12TNF |
| 1153 | CAD ~ Intercept.Low + AdipA1 + NTproBNP |
| 1154 | CAD ~ Intercept.Low + AdipA1 + PlGF |
| 1155 | CAD ~ Intercept.Low + NTproBNP + PlGF |
| 1156 | CAD ~ Intercept.Low + AdipA1 + A8MPO |
| 1157 | CAD ~ Intercept.Low + AdipA1 + A12TNF |
| 1158 | CAD ~ Intercept.Low + NTproBNP + A8MPO |
| 1159 | CAD ~ Intercept.Low + NTproBNP + A12TNF |
| 1160 | CAD ~ Intercept.Low + PlGF + A8MPO |
| 1161 | CAD ~ Intercept.Low + PlGF + A12TNF |
| 1162 | CAD ~ Intercept.Low + AdipA1 + NTproBNP + PlGF |
| 1163 | CAD ~ Intercept.Low + AdipA1 + NTproBNP + A8MPO |
| 1164 | CAD ~ Intercept.Low + AdipA1 + NTproBNP + A12TNF |
| 1165 | CAD ~ Intercept.Low + AdipA1 + PlGF + A8MPO |
| 1166 | CAD ~ Intercept.Low + AdipA1 + PlGF + A12TNF |
| 1167 | CAD ~ Intercept.Low + AdipA1 + A8MPO + A12TNF |
| 1168 | CAD ~ Intercept.Low + NTproBNP + PlGF + A8MPO |
| 1169 | CAD ~ Intercept.Low + NTproBNP + PlGF + A12TNF |
| 1170 | CAD ~ Intercept.Low + NTproBNP + A8MPO + A12TNF |
| 1171 | CAD ~ Intercept.Low + PlGF + A8MPO + A12TNF |
| 1172 | CAD ~ Intercept.Low + NTproBNP |
| 1173 | CAD ~ Intercept.Low + AdipA1 |
| 1174 | CAD ~ Intercept.Low + PlGF |
| 1175 | CAD ~ Intercept.Low + A8MPO |
| 1176 | CAD ~ Intercept.Low + A12TNF |
| 1177 | CAD ~ Intercept.Low + A8MPO + A12TNF |
| 1178 | CAD ~ Intercept.Low + AdipA1 + NTproBNP |
| 1179 | CAD ~ Intercept.Low + AdipA1 + PlGF |
| 1180 | CAD ~ Intercept.Low + NTproBNP + PlGF |
| 1181 | CAD ~ Intercept.Low + AdipA1 + A8MPO |
| 1182 | CAD ~ Intercept.Low + AdipA1 + A12TNF |
| 1183 | CAD ~ Intercept.Low + NTproBNP + A8MPO |
| 1184 | CAD ~ Intercept.Low + NTproBNP + A12TNF |
| 1185 | CAD ~ Intercept.Low + PlGF + A8MPO |
| 1186 | CAD ~ Intercept.Low + PlGF + A12TNF |
| 1187 | CAD ~ Intercept.Low + A8MPO + A12TNF |
| 1188 | CAD ~ Intercept.Low + AdipA1 + NTproBNP |
| 1189 | CAD ~ Intercept.Low + AdipA1 + PlGF |
| 1190 | CAD ~ Intercept.Low + NTproBNP + PlGF |
| 1191 | CAD ~ Intercept.Low + AdipA1 + A8MPO |
| 1192 | CAD ~ Intercept.Low + AdipA1 + A12TNF |
| 1193 | CAD ~ Intercept.Low + NTproBNP + A8MPO |
| 1194 | CAD ~ Intercept.Low + NTproBNP + A12TNF |
| 1195 | CAD ~ Intercept.Low + PlGF + A8MPO |
| 1196 | CAD ~ Intercept.Low + PlGF + A12TNF |
| 1197 | CAD ~ Intercept.Low + AdipA1 + NTproBNP + PlGF |
| 1198 | CAD ~ Intercept.Low + AdipA1 + NTproBNP + A8MPO |
| 1199 | CAD ~ Intercept.Low + AdipA1 + NTproBNP + A12TNF |
| 1200 | CAD ~ Intercept.Low + AdipA1 + PlGF + A8MPO |
| 1201 | CAD ~ Intercept.Low + AdipA1 + PlGF + A12TNF |
| 1202 | CAD ~ Intercept.Low + AdipA1 + A8MPO + A12TNF |
| 1203 | CAD ~ Intercept.Low + NTproBNP + PlGF + A8MPO |
| 1204 | CAD ~ Intercept.Low + NTproBNP + PlGF + A12TNF |
| 1205 | CAD ~ Intercept.Low + NTproBNP + A8MPO + A12TNF |
| 1206 | CAD ~ Intercept.Low + PlGF + A8MPO + A12TNF |
| 1207 | CAD ~ Intercept.Low + A8MPO + A12TNF |
| 1208 | CAD ~ Intercept.Low + AdipA1 + NTproBNP |
| 1209 | CAD ~ Intercept.Low + AdipA1 + PlGF |
| 1210 | CAD ~ Intercept.Low + NTproBNP + PlGF |
| 1211 | CAD ~ Intercept.Low + AdipA1 + A8MPO |
| 1212 | CAD ~ Intercept.Low + AdipA1 + A12TNF |
| 1213 | CAD ~ Intercept.Low + NTproBNP + A8MPO |
| 1214 | CAD ~ Intercept.Low + NTproBNP + A12TNF |
| 1215 | CAD ~ Intercept.Low + PlGF + A8MPO |
| 1216 | CAD ~ Intercept.Low + PlGF + A12TNF |
| 1217 | CAD ~ Intercept.Low + AdipA1 + NTproBNP + PlGF |
| 1218 | CAD ~ Intercept.Low + AdipA1 + NTproBNP + A8MPO |
| 1219 | CAD ~ Intercept.Low + AdipA1 + NTproBNP + A12TNF |
| 1220 | CAD ~ Intercept.Low + AdipA1 + PlGF + A8MPO |
| 1221 | CAD ~ Intercept.Low + AdipA1 + PlGF + A12TNF |
| 1222 | CAD ~ Intercept.Low + AdipA1 + A8MPO + A12TNF |
| 1223 | CAD ~ Intercept.Low + NTproBNP + PlGF + A8MPO |
| 1224 | CAD ~ Intercept.Low + NTproBNP + PlGF + A12TNF |
| 1225 | CAD ~ Intercept.Low + NTproBNP + A8MPO + A12TNF |
| 1226 | CAD ~ Intercept.Low + PlGF + A8MPO + A12TNF |
| 1227 | CAD ~ Intercept.Low + AdipA1 + NTproBNP + PlGF |
| 1228 | CAD ~ Intercept.Low + AdipA1 + NTproBNP + A8MPO |
| 1229 | CAD ~ Intercept.Low + AdipA1 + NTproBNP + A12TNF |
| 1230 | CAD ~ Intercept.Low + AdipA1 + PlGF + A8MPO |
| 1231 | CAD ~ Intercept.Low + AdipA1 + PlGF + A12TNF |
| 1232 | CAD ~ Intercept.Low + AdipA1 + A8MPO + A12TNF |
| 1233 | CAD ~ Intercept.Low + NTproBNP + PlGF + A8MPO |
| 1234 | CAD ~ Intercept.Low + NTproBNP + PlGF + A12TNF |
| 1235 | CAD ~ Intercept.Low + NTproBNP + A8MPO + A12TNF |
| 1236 | CAD ~ Intercept.Low + PlGF + A8MPO + A12TNF |
| 1237 | CAD ~ Intercept.Low + AdipA1 + NTproBNP + PlGF + A8MPO |
| 1238 | CAD ~ Intercept.Low + AdipA1 + NTproBNP + PlGF + A12TNF |
| 1239 | CAD ~ Intercept.Low + AdipA1 + NTproBNP + A8MPO + A12TNF |
| 1240 | CAD ~ Intercept.Low + AdipA1 + PlGF + A8MPO + A12TNF |
| 1241 | CAD ~ Intercept.Low + NTproBNP + PlGF + A8MPO + A12TNF |
| 1242 | CAD ~ Intercept.Low + NTproBNP |
| 1243 | CAD ~ Intercept.Low + AdipA1 |
| 1244 | CAD ~ Intercept.Low + PlGF |
| 1245 | CAD ~ Intercept.Low + A8MPO |
| 1246 | CAD ~ Intercept.Low + A12TNF |
| 1247 | CAD ~ Intercept.Low + A8MPO + A12TNF |
| 1248 | CAD ~ Intercept.Low + AdipA1 + NTproBNP |
| 1249 | CAD ~ Intercept.Low + AdipA1 + PlGF |
| 1250 | CAD ~ Intercept.Low + NTproBNP + PlGF |
| 1251 | CAD ~ Intercept.Low + AdipA1 + A8MPO |
| 1252 | CAD ~ Intercept.Low + AdipA1 + A12TNF |
| 1253 | CAD ~ Intercept.Low + NTproBNP + A8MPO |
| 1254 | CAD ~ Intercept.Low + NTproBNP + A12TNF |
| 1255 | CAD ~ Intercept.Low + PlGF + A8MPO |
| 1256 | CAD ~ Intercept.Low + PlGF + A12TNF |
| 1257 | CAD ~ Intercept.Low + A8MPO + A12TNF |
| 1258 | CAD ~ Intercept.Low + AdipA1 + NTproBNP |
| 1259 | CAD ~ Intercept.Low + AdipA1 + PlGF |
| 1260 | CAD ~ Intercept.Low + NTproBNP + PlGF |
| 1261 | CAD ~ Intercept.Low + AdipA1 + A8MPO |
| 1262 | CAD ~ Intercept.Low + AdipA1 + A12TNF |
| 1263 | CAD ~ Intercept.Low + NTproBNP + A8MPO |
| 1264 | CAD ~ Intercept.Low + NTproBNP + A12TNF |
| 1265 | CAD ~ Intercept.Low + PlGF + A8MPO |
| 1266 | CAD ~ Intercept.Low + PlGF + A12TNF |
| 1267 | CAD ~ Intercept.Low + AdipA1 + NTproBNP + PlGF |
| 1268 | CAD ~ Intercept.Low + AdipA1 + NTproBNP + A8MPO |
| 1269 | CAD ~ Intercept.Low + AdipA1 + NTproBNP + A12TNF |
| 1270 | CAD ~ Intercept.Low + AdipA1 + PlGF + A8MPO |
| 1271 | CAD ~ Intercept.Low + AdipA1 + PlGF + A12TNF |
| 1272 | CAD ~ Intercept.Low + AdipA1 + A8MPO + A12TNF |
| 1273 | CAD ~ Intercept.Low + NTproBNP + PlGF + A8MPO |
| 1274 | CAD ~ Intercept.Low + NTproBNP + PlGF + A12TNF |
| 1275 | CAD ~ Intercept.Low + NTproBNP + A8MPO + A12TNF |
| 1276 | CAD ~ Intercept.Low + PlGF + A8MPO + A12TNF |
| 1277 | CAD ~ Intercept.Low + A8MPO + A12TNF |
| 1278 | CAD ~ Intercept.Low + AdipA1 + NTproBNP |
| 1279 | CAD ~ Intercept.Low + AdipA1 + PlGF |
| 1280 | CAD ~ Intercept.Low + NTproBNP + PlGF |
| 1281 | CAD ~ Intercept.Low + AdipA1 + A8MPO |
| 1282 | CAD ~ Intercept.Low + AdipA1 + A12TNF |
| 1283 | CAD ~ Intercept.Low + NTproBNP + A8MPO |
| 1284 | CAD ~ Intercept.Low + NTproBNP + A12TNF |
| 1285 | CAD ~ Intercept.Low + PlGF + A8MPO |
| 1286 | CAD ~ Intercept.Low + PlGF + A12TNF |
| 1287 | CAD ~ Intercept.Low + AdipA1 + NTproBNP + PlGF |
| 1288 | CAD ~ Intercept.Low + AdipA1 + NTproBNP + A8MPO |
| 1289 | CAD ~ Intercept.Low + AdipA1 + NTproBNP + A12TNF |
| 1290 | CAD ~ Intercept.Low + AdipA1 + PlGF + A8MPO |
| 1291 | CAD ~ Intercept.Low + AdipA1 + PlGF + A12TNF |
| 1292 | CAD ~ Intercept.Low + AdipA1 + A8MPO + A12TNF |
| 1293 | CAD ~ Intercept.Low + NTproBNP + PlGF + A8MPO |
| 1294 | CAD ~ Intercept.Low + NTproBNP + PlGF + A12TNF |
| 1295 | CAD ~ Intercept.Low + NTproBNP + A8MPO + A12TNF |
| 1296 | CAD ~ Intercept.Low + PlGF + A8MPO + A12TNF |
| 1297 | CAD ~ Intercept.Low + AdipA1 + NTproBNP + PlGF |
| 1298 | CAD ~ Intercept.Low + AdipA1 + NTproBNP + A8MPO |
| 1299 | CAD ~ Intercept.Low + AdipA1 + NTproBNP + A12TNF |
| 1300 | CAD ~ Intercept.Low + AdipA1 + PlGF + A8MPO |
| 1301 | CAD ~ Intercept.Low + AdipA1 + PlGF + A12TNF |
| 1302 | CAD ~ Intercept.Low + AdipA1 + A8MPO + A12TNF |
| 1303 | CAD ~ Intercept.Low + NTproBNP + PlGF + A8MPO |
| 1304 | CAD ~ Intercept.Low + NTproBNP + PlGF + A12TNF |
| 1305 | CAD ~ Intercept.Low + NTproBNP + A8MPO + A12TNF |
| 1306 | CAD ~ Intercept.Low + PlGF + A8MPO + A12TNF |
| 1307 | CAD ~ Intercept.Low + AdipA1 + NTproBNP + PlGF + A8MPO |
| 1308 | CAD ~ Intercept.Low + AdipA1 + NTproBNP + PlGF + A12TNF |
| 1309 | CAD ~ Intercept.Low + AdipA1 + NTproBNP + A8MPO + A12TNF |
| 1310 | CAD ~ Intercept.Low + AdipA1 + PlGF + A8MPO + A12TNF |
| 1311 | CAD ~ Intercept.Low + NTproBNP + PlGF + A8MPO + A12TNF |
| 1312 | CAD ~ Intercept.Low + A8MPO + A12TNF |
| 1313 | CAD ~ Intercept.Low + AdipA1 + NTproBNP |
| 1314 | CAD ~ Intercept.Low + AdipA1 + PlGF |
| 1315 | CAD ~ Intercept.Low + NTproBNP + PlGF |
| 1316 | CAD ~ Intercept.Low + AdipA1 + A8MPO |
| 1317 | CAD ~ Intercept.Low + AdipA1 + A12TNF |
| 1318 | CAD ~ Intercept.Low + NTproBNP + A8MPO |
| 1319 | CAD ~ Intercept.Low + NTproBNP + A12TNF |
| 1320 | CAD ~ Intercept.Low + PlGF + A8MPO |
| 1321 | CAD ~ Intercept.Low + PlGF + A12TNF |
| 1322 | CAD ~ Intercept.Low + AdipA1 + NTproBNP + PlGF |
| 1323 | CAD ~ Intercept.Low + AdipA1 + NTproBNP + A8MPO |
| 1324 | CAD ~ Intercept.Low + AdipA1 + NTproBNP + A12TNF |
| 1325 | CAD ~ Intercept.Low + AdipA1 + PlGF + A8MPO |
| 1326 | CAD ~ Intercept.Low + AdipA1 + PlGF + A12TNF |
| 1327 | CAD ~ Intercept.Low + AdipA1 + A8MPO + A12TNF |
| 1328 | CAD ~ Intercept.Low + NTproBNP + PlGF + A8MPO |
| 1329 | CAD ~ Intercept.Low + NTproBNP + PlGF + A12TNF |
| 1330 | CAD ~ Intercept.Low + NTproBNP + A8MPO + A12TNF |
| 1331 | CAD ~ Intercept.Low + PlGF + A8MPO + A12TNF |
| 1332 | CAD ~ Intercept.Low + AdipA1 + NTproBNP + PlGF |
| 1333 | CAD ~ Intercept.Low + AdipA1 + NTproBNP + A8MPO |
| 1334 | CAD ~ Intercept.Low + AdipA1 + NTproBNP + A12TNF |
| 1335 | CAD ~ Intercept.Low + AdipA1 + PlGF + A8MPO |
| 1336 | CAD ~ Intercept.Low + AdipA1 + PlGF + A12TNF |
| 1337 | CAD ~ Intercept.Low + AdipA1 + A8MPO + A12TNF |
| 1338 | CAD ~ Intercept.Low + NTproBNP + PlGF + A8MPO |
| 1339 | CAD ~ Intercept.Low + NTproBNP + PlGF + A12TNF |
| 1340 | CAD ~ Intercept.Low + NTproBNP + A8MPO + A12TNF |
| 1341 | CAD ~ Intercept.Low + PlGF + A8MPO + A12TNF |
| 1342 | CAD ~ Intercept.Low + AdipA1 + NTproBNP + PlGF + A8MPO |
| 1343 | CAD ~ Intercept.Low + AdipA1 + NTproBNP + PlGF + A12TNF |
| 1344 | CAD ~ Intercept.Low + AdipA1 + NTproBNP + A8MPO + A12TNF |
| 1345 | CAD ~ Intercept.Low + AdipA1 + PlGF + A8MPO + A12TNF |
| 1346 | CAD ~ Intercept.Low + NTproBNP + PlGF + A8MPO + A12TNF |
| 1347 | CAD ~ Intercept.Low + AdipA1 + NTproBNP + PlGF |
| 1348 | CAD ~ Intercept.Low + AdipA1 + NTproBNP + A8MPO |
| 1349 | CAD ~ Intercept.Low + AdipA1 + NTproBNP + A12TNF |
| 1350 | CAD ~ Intercept.Low + AdipA1 + PlGF + A8MPO |
| 1351 | CAD ~ Intercept.Low + AdipA1 + PlGF + A12TNF |
| 1352 | CAD ~ Intercept.Low + AdipA1 + A8MPO + A12TNF |
| 1353 | CAD ~ Intercept.Low + NTproBNP + PlGF + A8MPO |
| 1354 | CAD ~ Intercept.Low + NTproBNP + PlGF + A12TNF |
| 1355 | CAD ~ Intercept.Low + NTproBNP + A8MPO + A12TNF |
| 1356 | CAD ~ Intercept.Low + PlGF + A8MPO + A12TNF |
| 1357 | CAD ~ Intercept.Low + AdipA1 + NTproBNP + PlGF + A8MPO |
| 1358 | CAD ~ Intercept.Low + AdipA1 + NTproBNP + PlGF + A12TNF |
| 1359 | CAD ~ Intercept.Low + AdipA1 + NTproBNP + A8MPO + A12TNF |
| 1360 | CAD ~ Intercept.Low + AdipA1 + PlGF + A8MPO + A12TNF |
| 1361 | CAD ~ Intercept.Low + NTproBNP + PlGF + A8MPO + A12TNF |
| 1362 | CAD ~ Intercept.Low + AdipA1 + NTproBNP + PlGF + A8MPO |
| 1363 | CAD ~ Intercept.Low + AdipA1 + NTproBNP + PlGF + A12TNF |
| 1364 | CAD ~ Intercept.Low + AdipA1 + NTproBNP + A8MPO + A12TNF |
| 1365 | CAD ~ Intercept.Low + AdipA1 + PlGF + A8MPO + A12TNF |
| 1366 | CAD ~ Intercept.Low + NTproBNP + PlGF + A8MPO + A12TNF |
| 1367 | CAD ~ Intercept.Low + AdipA1 + NTproBNP + PlGF + A8MPO + A12TNF |
| 1368 | CAD ~ Intercept.Low + NTproBNP |
| 1369 | CAD ~ Intercept.Low + AdipA1 |
| 1370 | CAD ~ Intercept.Low + PlGF |
| 1371 | CAD ~ Intercept.Low + A8MPO |
| 1372 | CAD ~ Intercept.Low + A12TNF |
| 1373 | CAD ~ Intercept.Low + A8MPO + A12TNF |
| 1374 | CAD ~ Intercept.Low + AdipA1 + NTproBNP |
| 1375 | CAD ~ Intercept.Low + AdipA1 + PlGF |
| 1376 | CAD ~ Intercept.Low + NTproBNP + PlGF |
| 1377 | CAD ~ Intercept.Low + AdipA1 + A8MPO |
| 1378 | CAD ~ Intercept.Low + AdipA1 + A12TNF |
| 1379 | CAD ~ Intercept.Low + NTproBNP + A8MPO |
| 1380 | CAD ~ Intercept.Low + NTproBNP + A12TNF |
| 1381 | CAD ~ Intercept.Low + PlGF + A8MPO |
| 1382 | CAD ~ Intercept.Low + PlGF + A12TNF |
| 1383 | CAD ~ Intercept.Low + A8MPO + A12TNF |
| 1384 | CAD ~ Intercept.Low + AdipA1 + NTproBNP |
| 1385 | CAD ~ Intercept.Low + AdipA1 + PlGF |
| 1386 | CAD ~ Intercept.Low + NTproBNP + PlGF |
| 1387 | CAD ~ Intercept.Low + AdipA1 + A8MPO |
| 1388 | CAD ~ Intercept.Low + AdipA1 + A12TNF |
| 1389 | CAD ~ Intercept.Low + NTproBNP + A8MPO |
| 1390 | CAD ~ Intercept.Low + NTproBNP + A12TNF |
| 1391 | CAD ~ Intercept.Low + PlGF + A8MPO |
| 1392 | CAD ~ Intercept.Low + PlGF + A12TNF |
| 1393 | CAD ~ Intercept.Low + AdipA1 + NTproBNP + PlGF |
| 1394 | CAD ~ Intercept.Low + AdipA1 + NTproBNP + A8MPO |
| 1395 | CAD ~ Intercept.Low + AdipA1 + NTproBNP + A12TNF |
| 1396 | CAD ~ Intercept.Low + AdipA1 + PlGF + A8MPO |
| 1397 | CAD ~ Intercept.Low + AdipA1 + PlGF + A12TNF |
| 1398 | CAD ~ Intercept.Low + AdipA1 + A8MPO + A12TNF |
| 1399 | CAD ~ Intercept.Low + NTproBNP + PlGF + A8MPO |
| 1400 | CAD ~ Intercept.Low + NTproBNP + PlGF + A12TNF |
| 1401 | CAD ~ Intercept.Low + NTproBNP + A8MPO + A12TNF |
| 1402 | CAD ~ Intercept.Low + PlGF + A8MPO + A12TNF |
| 1403 | CAD ~ Intercept.Low + A8MPO + A12TNF |
| 1404 | CAD ~ Intercept.Low + AdipA1 + NTproBNP |
| 1405 | CAD ~ Intercept.Low + AdipA1 + PlGF |
| 1406 | CAD ~ Intercept.Low + AdipA1 + A8MPO |
| 1407 | CAD ~ Intercept.Low + NTproBNP + PlGF |
| 1408 | CAD ~ Intercept.Low + AdipA1 + A12TNF |
| 1409 | CAD ~ Intercept.Low + NTproBNP + A8MPO |
| 1410 | CAD ~ Intercept.Low + NTproBNP + A12TNF |
| 1411 | CAD ~ Intercept.Low + PlGF + A8MPO |
| 1412 | CAD ~ Intercept.Low + PlGF + A12TNF |
| 1413 | CAD ~ Intercept.Low + AdipA1 + NTproBNP + PlGF |
| 1414 | CAD ~ Intercept.Low + AdipA1 + NTproBNP + A8MPO |
| 1415 | CAD ~ Intercept.Low + AdipA1 + NTproBNP + A12TNF |
| 1416 | CAD ~ Intercept.Low + AdipA1 + PlGF + A8MPO |
| 1417 | CAD ~ Intercept.Low + AdipA1 + PlGF + A12TNF |
| 1418 | CAD ~ Intercept.Low + AdipA1 + A8MPO + A12TNF |
| 1419 | CAD ~ Intercept.Low + NTproBNP + PlGF + A8MPO |
| 1420 | CAD ~ Intercept.Low + NTproBNP + PlGF + A12TNF |
| 1421 | CAD ~ Intercept.Low + NTproBNP + A8MPO + A12TNF |
| 1422 | CAD ~ Intercept.Low + PlGF + A8MPO + A12TNF |
| 1423 | CAD ~ Intercept.Low + AdipA1 + NTproBNP + PlGF |
| 1424 | CAD ~ Intercept.Low + AdipA1 + NTproBNP + A8MPO |
| 1425 | CAD ~ Intercept.Low + AdipA1 + NTproBNP + A12TNF |
| 1426 | CAD ~ Intercept.Low + AdipA1 + PlGF + A8MPO |
| 1427 | CAD ~ Intercept.Low + AdipA1 + PlGF + A12TNF |
| 1428 | CAD ~ Intercept.Low + AdipA1 + A8MPO + A12TNF |
| 1429 | CAD ~ Intercept.Low + NTproBNP + PlGF + A8MPO |
| 1430 | CAD ~ Intercept.Low + NTproBNP + PlGF + A12TNF |
| 1431 | CAD ~ Intercept.Low + NTproBNP + A8MPO + A12TNF |
| 1432 | CAD ~ Intercept.Low + PlGF + A8MPO + A12TNF |
| 1433 | CAD ~ Intercept.Low + AdipA1 + NTproBNP + PlGF + A8MPO |
| 1434 | CAD ~ Intercept.Low + AdipA1 + NTproBNP + PlGF + A12TNF |
| 1435 | CAD ~ Intercept.Low + AdipA1 + NTproBNP + A8MPO + A12TNF |
| 1436 | CAD ~ Intercept.Low + AdipA1 + PlGF + A8MPO + A12TNF |
| 1437 | CAD ~ Intercept.Low + NTproBNP + PlGF + A8MPO + A12TNF |
| 1438 | CAD ~ Intercept.Low + A8MPO + A12TNF |
| 1439 | CAD ~ Intercept.Low + AdipA1 + NTproBNP |
| 1440 | CAD ~ Intercept.Low + AdipA1 + PlGF |
| 1441 | CAD ~ Intercept.Low + AdipA1 + A8MPO |
| 1442 | CAD ~ Intercept.Low + NTproBNP + PlGF |
| 1443 | CAD ~ Intercept.Low + AdipA1 + A12TNF |
| 1444 | CAD ~ Intercept.Low + NTproBNP + A8MPO |
| 1445 | CAD ~ Intercept.Low + NTproBNP + A12TNF |
| 1446 | CAD ~ Intercept.Low + PlGF + A8MPO |
| 1447 | CAD ~ Intercept.Low + PlGF + A12TNF |
| 1448 | CAD ~ Intercept.Low + AdipA1 + NTproBNP + PlGF |
| 1449 | CAD ~ Intercept.Low + AdipA1 + NTproBNP + A8MPO |
| 1450 | CAD ~ Intercept.Low + AdipA1 + NTproBNP + A12TNF |
| 1451 | CAD ~ Intercept.Low + AdipA1 + PlGF + A8MPO |
| 1452 | CAD ~ Intercept.Low + AdipA1 + PlGF + A12TNF |
| 1453 | CAD ~ Intercept.Low + AdipA1 + A8MPO + A12TNF |
| 1454 | CAD ~ Intercept.Low + NTproBNP + PlGF + A8MPO |
| 1455 | CAD ~ Intercept.Low + NTproBNP + PlGF + A12TNF |
| 1456 | CAD ~ Intercept.Low + NTproBNP + A8MPO + A12TNF |
| 1457 | CAD ~ Intercept.Low + PlGF + A8MPO + A12TNF |
| 1458 | CAD ~ Intercept.Low + AdipA1 + NTproBNP + PlGF |
| 1459 | CAD ~ Intercept.Low + AdipA1 + NTproBNP + A8MPO |
| 1460 | CAD ~ Intercept.Low + AdipA1 + NTproBNP + A12TNF |
| 1461 | CAD ~ Intercept.Low + AdipA1 + PlGF + A8MPO |
| 1462 | CAD ~ Intercept.Low + AdipA1 + PlGF + A12TNF |
| 1463 | CAD ~ Intercept.Low + AdipA1 + A8MPO + A12TNF |
| 1464 | CAD ~ Intercept.Low + NTproBNP + PlGF + A8MPO |
| 1465 | CAD ~ Intercept.Low + NTproBNP + PlGF + A12TNF |
| 1466 | CAD ~ Intercept.Low + NTproBNP + A8MPO + A12TNF |
| 1467 | CAD ~ Intercept.Low + PlGF + A8MPO + A12TNF |
| 1468 | CAD ~ Intercept.Low + AdipA1 + NTproBNP + PlGF + A8MPO |
| 1469 | CAD ~ Intercept.Low + AdipA1 + NTproBNP + PlGF + A12TNF |
| 1470 | CAD ~ Intercept.Low + AdipA1 + NTproBNP + A8MPO + A12TNF |
| 1471 | CAD ~ Intercept.Low + AdipA1 + PlGF + A8MPO + A12TNF |
| 1472 | CAD ~ Intercept.Low + NTproBNP + PlGF + A8MPO + A12TNF |
| 1473 | CAD ~ Intercept.Low + AdipA1 + NTproBNP + PlGF |
| 1474 | CAD ~ Intercept.Low + AdipA1 + NTproBNP + A8MPO |
| 1475 | CAD ~ Intercept.Low + AdipA1 + NTproBNP + A12TNF |
| 1476 | CAD ~ Intercept.Low + AdipA1 + PlGF + A8MPO |
| 1477 | CAD ~ Intercept.Low + AdipA1 + PlGF + A12TNF |
| 1478 | CAD ~ Intercept.Low + AdipA1 + A8MPO + A12TNF |
| 1479 | CAD ~ Intercept.Low + NTproBNP + PlGF + A8MPO |
| 1480 | CAD ~ Intercept.Low + NTproBNP + PlGF + A12TNF |
| 1481 | CAD ~ Intercept.Low + NTproBNP + A8MPO + A12TNF |
| 1482 | CAD ~ Intercept.Low + PlGF + A8MPO + A12TNF |
| 1483 | CAD ~ Intercept.Low + AdipA1 + NTproBNP + PlGF + A8MPO |
| 1484 | CAD ~ Intercept.Low + AdipA1 + NTproBNP + PlGF + A12TNF |
| 1485 | CAD ~ Intercept.Low + AdipA1 + NTproBNP + A8MPO + A12TNF |
| 1486 | CAD ~ Intercept.Low + AdipA1 + PlGF + A8MPO + A12TNF |
| 1487 | CAD ~ Intercept.Low + NTproBNP + PlGF + A8MPO + A12TNF |
| 1488 | CAD ~ Intercept.Low + AdipA1 + NTproBNP + PlGF + A8MPO |
| 1489 | CAD ~ Intercept.Low + AdipA1 + NTproBNP + PlGF + A12TNF |
| 1490 | CAD ~ Intercept.Low + AdipA1 + NTproBNP + A8MPO + A12TNF |
| 1491 | CAD ~ Intercept.Low + AdipA1 + PlGF + A8MPO + A12TNF |
| 1492 | CAD ~ Intercept.Low + NTproBNP + PlGF + A8MPO + A12TNF |
| 1493 | CAD ~ Intercept.Low + AdipA1 + NTproBNP + PlGF + A8MPO + A12TNF |
| 1494 | CAD ~ Intercept.Low + A8MPO + A12TNF |
| 1495 | CAD ~ Intercept.Low + AdipA1 + NTproBNP |
| 1496 | CAD ~ Intercept.Low + AdipA1 + PlGF |
| 1497 | CAD ~ Intercept.Low + AdipA1 + A8MPO |
| 1498 | CAD ~ Intercept.Low + NTproBNP + PlGF |
| 1499 | CAD ~ Intercept.Low + AdipA1 + A12TNF |
| 1500 | CAD ~ Intercept.Low + NTproBNP + A8MPO |
| 1501 | CAD ~ Intercept.Low + NTproBNP + A12TNF |
| 1502 | CAD ~ Intercept.Low + PlGF + A8MPO |
| 1503 | CAD ~ Intercept.Low + PlGF + A12TNF |
| 1504 | CAD ~ Intercept.Low + AdipA1 + NTproBNP + PlGF |
| 1505 | CAD ~ Intercept.Low + AdipA1 + NTproBNP + A8MPO |
| 1506 | CAD ~ Intercept.Low + AdipA1 + NTproBNP + A12TNF |
| 1507 | CAD ~ Intercept.Low + AdipA1 + PlGF + A8MPO |
| 1508 | CAD ~ Intercept.Low + AdipA1 + PlGF + A12TNF |
| 1509 | CAD ~ Intercept.Low + AdipA1 + A8MPO + A12TNF |
| 1510 | CAD ~ Intercept.Low + NTproBNP + PlGF + A8MPO |
| 1511 | CAD ~ Intercept.Low + NTproBNP + PlGF + A12TNF |
| 1512 | CAD ~ Intercept.Low + NTproBNP + A8MPO + A12TNF |
| 1513 | CAD ~ Intercept.Low + PlGF + A8MPO + A12TNF |
| 1514 | CAD ~ Intercept.Low + AdipA1 + NTproBNP + PlGF |
| 1515 | CAD ~ Intercept.Low + AdipA1 + NTproBNP + A8MPO |
| 1516 | CAD ~ Intercept.Low + AdipA1 + NTproBNP + A12TNF |
| 1517 | CAD ~ Intercept.Low + AdipA1 + PlGF + A8MPO |
| 1518 | CAD ~ Intercept.Low + AdipA1 + PlGF + A12TNF |
| 1519 | CAD ~ Intercept.Low + AdipA1 + A8MPO + A12TNF |
| 1520 | CAD ~ Intercept.Low + NTproBNP + PlGF + A8MPO |
| 1521 | CAD ~ Intercept.Low + NTproBNP + PlGF + A12TNF |
| 1522 | CAD ~ Intercept.Low + NTproBNP + A8MPO + A12TNF |
| 1523 | CAD ~ Intercept.Low + PlGF + A8MPO + A12TNF |
| 1524 | CAD ~ Intercept.Low + AdipA1 + NTproBNP + PlGF + A8MPO |
| 1525 | CAD ~ Intercept.Low + AdipA1 + NTproBNP + PlGF + A12TNF |
| 1526 | CAD ~ Intercept.Low + AdipA1 + NTproBNP + A8MPO + A12TNF |
| 1527 | CAD ~ Intercept.Low + AdipA1 + PlGF + A8MPO + A12TNF |
| 1528 | CAD ~ Intercept.Low + NTproBNP + PlGF + A8MPO + A12TNF |
| 1529 | CAD ~ Intercept.Low + AdipA1 + NTproBNP + PlGF |
| 1530 | CAD ~ Intercept.Low + AdipA1 + NTproBNP + A8MPO |
| 1531 | CAD ~ Intercept.Low + AdipA1 + NTproBNP + A12TNF |
| 1532 | CAD ~ Intercept.Low + AdipA1 + PlGF + A8MPO |
| 1533 | CAD ~ Intercept.Low + AdipA1 + PlGF + A12TNF |
| 1534 | CAD ~ Intercept.Low + AdipA1 + A8MPO + A12TNF |
| 1535 | CAD ~ Intercept.Low + NTproBNP + PlGF + A8MPO |
| 1536 | CAD ~ Intercept.Low + NTproBNP + PlGF + A12TNF |
| 1537 | CAD ~ Intercept.Low + NTproBNP + A8MPO + A12TNF |
| 1538 | CAD ~ Intercept.Low + PlGF + A8MPO + A12TNF |
| 1539 | CAD ~ Intercept.Low + AdipA1 + NTproBNP + PlGF + A8MPO |
| 1540 | CAD ~ Intercept.Low + AdipA1 + NTproBNP + PlGF + A12TNF |
| 1541 | CAD ~ Intercept.Low + AdipA1 + NTproBNP + A8MPO + A12TNF |
| 1542 | CAD ~ Intercept.Low + AdipA1 + PlGF + A8MPO + A12TNF |
| 1543 | CAD ~ Intercept.Low + NTproBNP + PlGF + A8MPO + A12TNF |
| 1544 | CAD ~ Intercept.Low + AdipA1 + NTproBNP + PlGF + A8MPO |
| 1545 | CAD ~ Intercept.Low + AdipA1 + NTproBNP + PlGF + A12TNF |
| 1546 | CAD ~ Intercept.Low + AdipA1 + NTproBNP + A8MPO + A12TNF |
| 1547 | CAD ~ Intercept.Low + AdipA1 + PlGF + A8MPO + A12TNF |
| 1548 | CAD ~ Intercept.Low + NTproBNP + PlGF + A8MPO + A12TNF |
| 1549 | CAD ~ Intercept.Low + AdipA1 + NTproBNP + PlGF + A8MPO + A12TNF |
| 1550 | CAD ~ Intercept.Low + AdipA1 + NTproBNP + PlGF |
| 1551 | CAD ~ Intercept.Low + AdipA1 + NTproBNP + A8MPO |
| 1552 | CAD ~ Intercept.Low + AdipA1 + NTproBNP + A12TNF |
| 1553 | CAD ~ Intercept.Low + AdipA1 + PlGF + A8MPO |
| 1554 | CAD ~ Intercept.Low + AdipA1 + PlGF + A12TNF |
| 1555 | CAD ~ Intercept.Low + AdipA1 + A8MPO + A12TNF |
| 1556 | CAD ~ Intercept.Low + NTproBNP + PlGF + A8MPO |
| 1557 | CAD ~ Intercept.Low + NTproBNP + PlGF + A12TNF |
| 1558 | CAD ~ Intercept.Low + NTproBNP + A8MPO + A12TNF |
| 1559 | CAD ~ Intercept.Low + PlGF + A8MPO + A12TNF |
| 1560 | CAD ~ Intercept.Low + AdipA1 + NTproBNP + PlGF + A8MPO |
| 1561 | CAD ~ Intercept.Low + AdipA1 + NTproBNP + PlGF + A12TNF |
| 1562 | CAD ~ Intercept.Low + AdipA1 + NTproBNP + A8MPO + A12TNF |
| 1563 | CAD ~ Intercept.Low + AdipA1 + PlGF + A8MPO + A12TNF |
| 1564 | CAD ~ Intercept.Low + NTproBNP + PlGF + A8MPO + A12TNF |
| 1565 | CAD ~ Intercept.Low + AdipA1 + NTproBNP + PlGF + A8MPO |
| 1566 | CAD ~ Intercept.Low + AdipA1 + NTproBNP + PlGF + A12TNF |
| 1567 | CAD ~ Intercept.Low + AdipA1 + NTproBNP + A8MPO + A12TNF |
| 1568 | CAD ~ Intercept.Low + AdipA1 + PlGF + A8MPO + A12TNF |
| 1569 | CAD ~ Intercept.Low + NTproBNP + PlGF + A8MPO + A12TNF |
| 1570 | CAD ~ Intercept.Low + AdipA1 + NTproBNP + PlGF + A8MPO + A12TNF |
| 1571 | CAD ~ Intercept.Low + AdipA1 + NTproBNP + PlGF + A8MPO |
| 1572 | CAD ~ Intercept.Low + AdipA1 + NTproBNP + PlGF + A12TNF |
| 1573 | CAD ~ Intercept.Low + AdipA1 + NTproBNP + A8MPO + A12TNF |
| 1574 | CAD ~ Intercept.Low + AdipA1 + PlGF + A8MPO + A12TNF |
| 1575 | CAD ~ Intercept.Low + NTproBNP + PlGF + A8MPO + A12TNF |
| 1576 | CAD ~ Intercept.Low + AdipA1 + NTproBNP + PlGF + A8MPO + A12TNF |
| 1577 | CAD ~ Intercept.Low + AdipA1 + NTproBNP + PlGF + A8MPO + A12TNF |
| 1578 | CAD ~ Intercept.Low + AdipA1 + NTproBNP + PlGF + A8MPO |
| 1579 | CAD ~ Intercept.Low + AdipA1 + NTproBNP + PlGF + A12TNF |
| 1580 | CAD ~ Intercept.Low + AdipA1 + NTproBNP + A8MPO + A12TNF |
| 1581 | CAD ~ Intercept.Low + AdipA1 + PlGF + A8MPO + A12TNF |
| 1582 | CAD ~ Intercept.Low + NTproBNP + PlGF + A8MPO + A12TNF |
| 1583 | CAD ~ Intercept.Low + AdipA1 + NTproBNP + PlGF + A8MPO + A12TNF |
| 1584 | CAD ~ Intercept.Low + AdipA1 + NTproBNP + PlGF + A8MPO + A12TNF |
| 1585 | CAD ~ Intercept.Low + AdipA1 + NTproBNP + PlGF + A8MPO + A12TNF |
| 1586 | CAD ~ Intercept.Low + NTproBNP |
| 1587 | CAD ~ Intercept.Low + AdipA1 |
| 1588 | CAD ~ Intercept.Low + PlGF |
| 1589 | CAD ~ Intercept.Low + A8MPO |
| 1590 | CAD ~ Intercept.Low + A12TNF |
| 1591 | CAD ~ Intercept.Low + NTproBNP |
| 1592 | CAD ~ Intercept.Low + AdipA1 |
| 1593 | CAD ~ Intercept.Low + PlGF |
| 1594 | CAD ~ Intercept.Low + A8MPO |
| 1595 | CAD ~ Intercept.Low + A12TNF |
| 1596 | CAD ~ Intercept.Low + A8MPO + A12TNF |
| 1597 | CAD ~ Intercept.Low + AdipA1 + NTproBNP |
| 1598 | CAD ~ Intercept.Low + NTproBNP + PlGF |
| 1599 | CAD ~ Intercept.Low + AdipA1 + PlGF |
| 1600 | CAD ~ Intercept.Low + AdipA1 + A8MPO |
| 1601 | CAD ~ Intercept.Low + AdipA1 + A12TNF |
| 1602 | CAD ~ Intercept.Low + NTproBNP + A8MPO |
| 1603 | CAD ~ Intercept.Low + NTproBNP + A12TNF |
| 1604 | CAD ~ Intercept.Low + PlGF + A8MPO |
| 1605 | CAD ~ Intercept.Low + PlGF + A12TNF |
| 1606 | CAD ~ Intercept.Low + NTproBNP |
| 1607 | CAD ~ Intercept.Low + AdipA1 |
| 1608 | CAD ~ Intercept.Low + PlGF |
| 1609 | CAD ~ Intercept.Low + A8MPO |
| 1610 | CAD ~ Intercept.Low + A12TNF |
| 1611 | CAD ~ Intercept.Low + A8MPO + A12TNF |
| 1612 | CAD ~ Intercept.Low + AdipA1 + NTproBNP |
| 1613 | CAD ~ Intercept.Low + NTproBNP + PlGF |
| 1614 | CAD ~ Intercept.Low + AdipA1 + PlGF |
| 1615 | CAD ~ Intercept.Low + AdipA1 + A8MPO |
| 1616 | CAD ~ Intercept.Low + AdipA1 + A12TNF |
| 1617 | CAD ~ Intercept.Low + NTproBNP + A8MPO |
| 1618 | CAD ~ Intercept.Low + NTproBNP + A12TNF |
| 1619 | CAD ~ Intercept.Low + PlGF + A8MPO |
| 1620 | CAD ~ Intercept.Low + PlGF + A12TNF |
| 1621 | CAD ~ Intercept.Low + A8MPO + A12TNF |
| 1622 | CAD ~ Intercept.Low + AdipA1 + NTproBNP |
| 1623 | CAD ~ Intercept.Low + NTproBNP + PlGF |
| 1624 | CAD ~ Intercept.Low + AdipA1 + PlGF |
| 1625 | CAD ~ Intercept.Low + AdipA1 + A8MPO |
| 1626 | CAD ~ Intercept.Low + AdipA1 + A12TNF |
| 1627 | CAD ~ Intercept.Low + NTproBNP + A8MPO |
| 1628 | CAD ~ Intercept.Low + NTproBNP + A12TNF |
| 1629 | CAD ~ Intercept.Low + PlGF + A8MPO |
| 1630 | CAD ~ Intercept.Low + PlGF + A12TNF |
| 1631 | CAD ~ Intercept.Low + AdipA1 + NTproBNP + PlGF |
| 1632 | CAD ~ Intercept.Low + AdipA1 + NTproBNP + A8MPO |
| 1633 | CAD ~ Intercept.Low + AdipA1 + NTproBNP + A12TNF |
| 1634 | CAD ~ Intercept.Low + AdipA1 + PlGF + A8MPO |
| 1635 | CAD ~ Intercept.Low + AdipA1 + PlGF + A12TNF |
| 1636 | CAD ~ Intercept.Low + AdipA1 + A8MPO + A12TNF |
| 1637 | CAD ~ Intercept.Low + NTproBNP + PlGF + A8MPO |
| 1638 | CAD ~ Intercept.Low + NTproBNP + PlGF + A12TNF |
| 1639 | CAD ~ Intercept.Low + NTproBNP + A8MPO + A12TNF |
| 1640 | CAD ~ Intercept.Low + PlGF + A8MPO + A12TNF |
| 1641 | CAD ~ Intercept.Low + NTproBNP |
| 1642 | CAD ~ Intercept.Low + AdipA1 |
| 1643 | CAD ~ Intercept.Low + PlGF |
| 1644 | CAD ~ Intercept.Low + A8MPO |
| 1645 | CAD ~ Intercept.Low + A12TNF |
| 1646 | CAD ~ Intercept.Low + A8MPO + A12TNF |
| 1647 | CAD ~ Intercept.Low + AdipA1 + NTproBNP |
| 1648 | CAD ~ Intercept.Low + NTproBNP + PlGF |
| 1649 | CAD ~ Intercept.Low + AdipA1 + PlGF |
| 1650 | CAD ~ Intercept.Low + AdipA1 + A8MPO |
| 1651 | CAD ~ Intercept.Low + AdipA1 + A12TNF |
| 1652 | CAD ~ Intercept.Low + NTproBNP + A8MPO |
| 1653 | CAD ~ Intercept.Low + NTproBNP + A12TNF |
| 1654 | CAD ~ Intercept.Low + PlGF + A8MPO |
| 1655 | CAD ~ Intercept.Low + PlGF + A12TNF |
| 1656 | CAD ~ Intercept.Low + A8MPO + A12TNF |
| 1657 | CAD ~ Intercept.Low + AdipA1 + NTproBNP |
| 1658 | CAD ~ Intercept.Low + NTproBNP + PlGF |
| 1659 | CAD ~ Intercept.Low + AdipA1 + PlGF |
| 1660 | CAD ~ Intercept.Low + AdipA1 + A8MPO |
| 1661 | CAD ~ Intercept.Low + AdipA1 + A12TNF |
| 1662 | CAD ~ Intercept.Low + NTproBNP + A8MPO |
| 1663 | CAD ~ Intercept.Low + NTproBNP + A12TNF |
| 1664 | CAD ~ Intercept.Low + PlGF + A8MPO |
| 1665 | CAD ~ Intercept.Low + PlGF + A12TNF |
| 1666 | CAD ~ Intercept.Low + AdipA1 + NTproBNP + PlGF |
| 1667 | CAD ~ Intercept.Low + AdipA1 + NTproBNP + A8MPO |
| 1668 | CAD ~ Intercept.Low + AdipA1 + NTproBNP + A12TNF |
| 1669 | CAD ~ Intercept.Low + AdipA1 + PlGF + A8MPO |
| 1670 | CAD ~ Intercept.Low + AdipA1 + PlGF + A12TNF |
| 1671 | CAD ~ Intercept.Low + AdipA1 + A8MPO + A12TNF |
| 1672 | CAD ~ Intercept.Low + NTproBNP + PlGF + A8MPO |
| 1673 | CAD ~ Intercept.Low + NTproBNP + PlGF + A12TNF |
| 1674 | CAD ~ Intercept.Low + NTproBNP + A8MPO + A12TNF |
| 1675 | CAD ~ Intercept.Low + PlGF + A8MPO + A12TNF |
| 1676 | CAD ~ Intercept.Low + A8MPO + A12TNF |
| 1677 | CAD ~ Intercept.Low + AdipA1 + NTproBNP |
| 1678 | CAD ~ Intercept.Low + NTproBNP + PlGF |
| 1679 | CAD ~ Intercept.Low + AdipA1 + PlGF |
| 1680 | CAD ~ Intercept.Low + AdipA1 + A8MPO |
| 1681 | CAD ~ Intercept.Low + AdipA1 + A12TNF |
| 1682 | CAD ~ Intercept.Low + NTproBNP + A8MPO |
| 1683 | CAD ~ Intercept.Low + NTproBNP + A12TNF |
| 1684 | CAD ~ Intercept.Low + PlGF + A8MPO |
| 1685 | CAD ~ Intercept.Low + PlGF + A12TNF |
| 1686 | CAD ~ Intercept.Low + AdipA1 + NTproBNP + PlGF |
| 1687 | CAD ~ Intercept.Low + AdipA1 + NTproBNP + A8MPO |
| 1688 | CAD ~ Intercept.Low + AdipA1 + NTproBNP + A12TNF |
| 1689 | CAD ~ Intercept.Low + AdipA1 + PlGF + A8MPO |
| 1690 | CAD ~ Intercept.Low + AdipA1 + PlGF + A12TNF |
| 1691 | CAD ~ Intercept.Low + AdipA1 + A8MPO + A12TNF |
| 1692 | CAD ~ Intercept.Low + NTproBNP + PlGF + A8MPO |
| 1693 | CAD ~ Intercept.Low + NTproBNP + PlGF + A12TNF |
| 1694 | CAD ~ Intercept.Low + NTproBNP + A8MPO + A12TNF |
| 1695 | CAD ~ Intercept.Low + PlGF + A8MPO + A12TNF |
| 1696 | CAD ~ Intercept.Low + AdipA1 + NTproBNP + PlGF |
| 1697 | CAD ~ Intercept.Low + AdipA1 + NTproBNP + A8MPO |
| 1698 | CAD ~ Intercept.Low + AdipA1 + NTproBNP + A12TNF |
| 1699 | CAD ~ Intercept.Low + AdipA1 + PlGF + A8MPO |
| 1700 | CAD ~ Intercept.Low + AdipA1 + PlGF + A12TNF |
| 1701 | CAD ~ Intercept.Low + AdipA1 + A8MPO + A12TNF |
| 1702 | CAD ~ Intercept.Low + NTproBNP + PlGF + A8MPO |
| 1703 | CAD ~ Intercept.Low + NTproBNP + PlGF + A12TNF |
| 1704 | CAD ~ Intercept.Low + NTproBNP + A8MPO + A12TNF |
| 1705 | CAD ~ Intercept.Low + PlGF + A8MPO + A12TNF |
| 1706 | CAD ~ Intercept.Low + AdipA1 + NTproBNP + PlGF + A8MPO |
| 1707 | CAD ~ Intercept.Low + AdipA1 + NTproBNP + PlGF + A12TNF |
| 1708 | CAD ~ Intercept.Low + AdipA1 + NTproBNP + A8MPO + A12TNF |
| 1709 | CAD ~ Intercept.Low + AdipA1 + PlGF + A8MPO + A12TNF |
| 1710 | CAD ~ Intercept.Low + NTproBNP + PlGF + A8MPO + A12TNF |
| 1711 | CAD ~ Intercept.Low + NTproBNP |
| 1712 | CAD ~ Intercept.Low + AdipA1 |
| 1713 | CAD ~ Intercept.Low + PlGF |
| 1714 | CAD ~ Intercept.Low + A8MPO |
| 1715 | CAD ~ Intercept.Low + A12TNF |
| 1716 | CAD ~ Intercept.Low + A8MPO + A12TNF |
| 1717 | CAD ~ Intercept.Low + AdipA1 + NTproBNP |
| 1718 | CAD ~ Intercept.Low + NTproBNP + PlGF |
| 1719 | CAD ~ Intercept.Low + AdipA1 + PlGF |
| 1720 | CAD ~ Intercept.Low + AdipA1 + A8MPO |
| 1721 | CAD ~ Intercept.Low + AdipA1 + A12TNF |
| 1722 | CAD ~ Intercept.Low + NTproBNP + A8MPO |
| 1723 | CAD ~ Intercept.Low + NTproBNP + A12TNF |
| 1724 | CAD ~ Intercept.Low + PlGF + A8MPO |
| 1725 | CAD ~ Intercept.Low + PlGF + A12TNF |
| 1726 | CAD ~ Intercept.Low + A8MPO + A12TNF |
| 1727 | CAD ~ Intercept.Low + AdipA1 + NTproBNP |
| 1728 | CAD ~ Intercept.Low + NTproBNP + PlGF |
| 1729 | CAD ~ Intercept.Low + AdipA1 + PlGF |
| 1730 | CAD ~ Intercept.Low + AdipA1 + A8MPO |
| 1731 | CAD ~ Intercept.Low + AdipA1 + A12TNF |
| 1732 | CAD ~ Intercept.Low + NTproBNP + A8MPO |
| 1733 | CAD ~ Intercept.Low + NTproBNP + A12TNF |
| 1734 | CAD ~ Intercept.Low + PlGF + A8MPO |
| 1735 | CAD ~ Intercept.Low + PlGF + A12TNF |
| 1736 | CAD ~ Intercept.Low + AdipA1 + NTproBNP + PlGF |
| 1737 | CAD ~ Intercept.Low + AdipA1 + NTproBNP + A8MPO |
| 1738 | CAD ~ Intercept.Low + AdipA1 + NTproBNP + A12TNF |
| 1739 | CAD ~ Intercept.Low + AdipA1 + PlGF + A8MPO |
| 1740 | CAD ~ Intercept.Low + AdipA1 + PlGF + A12TNF |
| 1741 | CAD ~ Intercept.Low + AdipA1 + A8MPO + A12TNF |
| 1742 | CAD ~ Intercept.Low + NTproBNP + PlGF + A8MPO |
| 1743 | CAD ~ Intercept.Low + NTproBNP + PlGF + A12TNF |
| 1744 | CAD ~ Intercept.Low + NTproBNP + A8MPO + A12TNF |
| 1745 | CAD ~ Intercept.Low + PlGF + A8MPO + A12TNF |
| 1746 | CAD ~ Intercept.Low + A8MPO + A12TNF |
| 1747 | CAD ~ Intercept.Low + AdipA1 + NTproBNP |
| 1748 | CAD ~ Intercept.Low + NTproBNP + PlGF |
| 1749 | CAD ~ Intercept.Low + AdipA1 + PlGF |
| 1750 | CAD ~ Intercept.Low + AdipA1 + A8MPO |
| 1751 | CAD ~ Intercept.Low + AdipA1 + A12TNF |
| 1752 | CAD ~ Intercept.Low + NTproBNP + A8MPO |
| 1753 | CAD ~ Intercept.Low + NTproBNP + A12TNF |
| 1754 | CAD ~ Intercept.Low + PlGF + A8MPO |
| 1755 | CAD ~ Intercept.Low + PlGF + A12TNF |
| 1756 | CAD ~ Intercept.Low + AdipA1 + NTproBNP + PlGF |
| 1757 | CAD ~ Intercept.Low + AdipA1 + NTproBNP + A8MPO |
| 1758 | CAD ~ Intercept.Low + AdipA1 + NTproBNP + A12TNF |
| 1759 | CAD ~ Intercept.Low + AdipA1 + PlGF + A8MPO |
| 1760 | CAD ~ Intercept.Low + AdipA1 + PlGF + A12TNF |
| 1761 | CAD ~ Intercept.Low + AdipA1 + A8MPO + A12TNF |
| 1762 | CAD ~ Intercept.Low + NTproBNP + PlGF + A8MPO |
| 1763 | CAD ~ Intercept.Low + NTproBNP + PlGF + A12TNF |
| 1764 | CAD ~ Intercept.Low + NTproBNP + A8MPO + A12TNF |
| 1765 | CAD ~ Intercept.Low + PlGF + A8MPO + A12TNF |
| 1766 | CAD ~ Intercept.Low + AdipA1 + NTproBNP + PlGF |
| 1767 | CAD ~ Intercept.Low + AdipA1 + NTproBNP + A8MPO |
| 1768 | CAD ~ Intercept.Low + AdipA1 + NTproBNP + A12TNF |
| 1769 | CAD ~ Intercept.Low + AdipA1 + PlGF + A8MPO |
| 1770 | CAD ~ Intercept.Low + AdipA1 + PlGF + A12TNF |
| 1771 | CAD ~ Intercept.Low + AdipA1 + A8MPO + A12TNF |
| 1772 | CAD ~ Intercept.Low + NTproBNP + PlGF + A8MPO |
| 1773 | CAD ~ Intercept.Low + NTproBNP + PlGF + A12TNF |
| 1774 | CAD ~ Intercept.Low + NTproBNP + A8MPO + A12TNF |
| 1775 | CAD ~ Intercept.Low + PlGF + A8MPO + A12TNF |
| 1776 | CAD ~ Intercept.Low + AdipA1 + NTproBNP + PlGF + A8MPO |
| 1777 | CAD ~ Intercept.Low + AdipA1 + NTproBNP + PlGF + A12TNF |
| 1778 | CAD ~ Intercept.Low + AdipA1 + NTproBNP + A8MPO + A12TNF |
| 1779 | CAD ~ Intercept.Low + AdipA1 + PlGF + A8MPO + A12TNF |
| 1780 | CAD ~ Intercept.Low + NTproBNP + PlGF + A8MPO + A12TNF |
| 1781 | CAD ~ Intercept.Low + A8MPO + A12TNF |
| 1782 | CAD ~ Intercept.Low + AdipA1 + NTproBNP |
| 1783 | CAD ~ Intercept.Low + NTproBNP + PlGF |
| 1784 | CAD ~ Intercept.Low + AdipA1 + PlGF |
| 1785 | CAD ~ Intercept.Low + AdipA1 + A8MPO |
| 1786 | CAD ~ Intercept.Low + AdipA1 + A12TNF |
| 1787 | CAD ~ Intercept.Low + NTproBNP + A8MPO |
| 1788 | CAD ~ Intercept.Low + NTproBNP + A12TNF |
| 1789 | CAD ~ Intercept.Low + PlGF + A8MPO |
| 1790 | CAD ~ Intercept.Low + PlGF + A12TNF |
| 1791 | CAD ~ Intercept.Low + AdipA1 + NTproBNP + PlGF |
| 1792 | CAD ~ Intercept.Low + AdipA1 + NTproBNP + A8MPO |
| 1793 | CAD ~ Intercept.Low + AdipA1 + NTproBNP + A12TNF |
| 1794 | CAD ~ Intercept.Low + AdipA1 + PlGF + A8MPO |
| 1795 | CAD ~ Intercept.Low + AdipA1 + PlGF + A12TNF |
| 1796 | CAD ~ Intercept.Low + AdipA1 + A8MPO + A12TNF |
| 1797 | CAD ~ Intercept.Low + NTproBNP + PlGF + A8MPO |
| 1798 | CAD ~ Intercept.Low + NTproBNP + PlGF + A12TNF |
| 1799 | CAD ~ Intercept.Low + NTproBNP + A8MPO + A12TNF |
| 1800 | CAD ~ Intercept.Low + PlGF + A8MPO + A12TNF |
| 1801 | CAD ~ Intercept.Low + AdipA1 + NTproBNP + PlGF |
| 1802 | CAD ~ Intercept.Low + AdipA1 + NTproBNP + A8MPO |
| 1803 | CAD ~ Intercept.Low + AdipA1 + NTproBNP + A12TNF |
| 1804 | CAD ~ Intercept.Low + AdipA1 + PlGF + A8MPO |
| 1805 | CAD ~ Intercept.Low + AdipA1 + PlGF + A12TNF |
| 1806 | CAD ~ Intercept.Low + AdipA1 + A8MPO + A12TNF |
| 1807 | CAD ~ Intercept.Low + NTproBNP + PlGF + A8MPO |
| 1808 | CAD ~ Intercept.Low + NTproBNP + PlGF + A12TNF |
| 1809 | CAD ~ Intercept.Low + NTproBNP + A8MPO + A12TNF |
| 1810 | CAD ~ Intercept.Low + PlGF + A8MPO + A12TNF |
| 1811 | CAD ~ Intercept.Low + AdipA1 + NTproBNP + PlGF + A8MPO |
| 1812 | CAD ~ Intercept.Low + AdipA1 + NTproBNP + PlGF + A12TNF |
| 1813 | CAD ~ Intercept.Low + AdipA1 + NTproBNP + A8MPO + A12TNF |
| 1814 | CAD ~ Intercept.Low + AdipA1 + PlGF + A8MPO + A12TNF |
| 1815 | CAD ~ Intercept.Low + NTproBNP + PlGF + A8MPO + A12TNF |
| 1816 | CAD ~ Intercept.Low + AdipA1 + NTproBNP + PlGF |
| 1817 | CAD ~ Intercept.Low + AdipA1 + NTproBNP + A8MPO |
| 1818 | CAD ~ Intercept.Low + AdipA1 + NTproBNP + A12TNF |
| 1819 | CAD ~ Intercept.Low + AdipA1 + PlGF + A8MPO |
| 1820 | CAD ~ Intercept.Low + AdipA1 + PlGF + A12TNF |
| 1821 | CAD ~ Intercept.Low + AdipA1 + A8MPO + A12TNF |
| 1822 | CAD ~ Intercept.Low + NTproBNP + PlGF + A8MPO |
| 1823 | CAD ~ Intercept.Low + NTproBNP + PlGF + A12TNF |
| 1824 | CAD ~ Intercept.Low + NTproBNP + A8MPO + A12TNF |
| 1825 | CAD ~ Intercept.Low + PlGF + A8MPO + A12TNF |
| 1826 | CAD ~ Intercept.Low + AdipA1 + NTproBNP + PlGF + A8MPO |
| 1827 | CAD ~ Intercept.Low + AdipA1 + NTproBNP + PlGF + A12TNF |
| 1828 | CAD ~ Intercept.Low + AdipA1 + NTproBNP + A8MPO + A12TNF |
| 1829 | CAD ~ Intercept.Low + AdipA1 + PlGF + A8MPO + A12TNF |
| 1830 | CAD ~ Intercept.Low + NTproBNP + PlGF + A8MPO + A12TNF |
| 1831 | CAD ~ Intercept.Low + AdipA1 + NTproBNP + PlGF + A8MPO |
| 1832 | CAD ~ Intercept.Low + AdipA1 + NTproBNP + PlGF + A12TNF |
| 1833 | CAD ~ Intercept.Low + AdipA1 + NTproBNP + A8MPO + A12TNF |
| 1834 | CAD ~ Intercept.Low + AdipA1 + PlGF + A8MPO + A12TNF |
| 1835 | CAD ~ Intercept.Low + NTproBNP + PlGF + A8MPO + A12TNF |
| 1836 | CAD ~ Intercept.Low + AdipA1 + NTproBNP + PlGF + A8MPO + A12TNF |
| 1837 | CAD ~ Intercept.Low + NTproBNP |
| 1838 | CAD ~ Intercept.Low + AdipA1 |
| 1839 | CAD ~ Intercept.Low + PlGF |
| 1840 | CAD ~ Intercept.Low + A8MPO |
| 1841 | CAD ~ Intercept.Low + A12TNF |
| 1842 | CAD ~ Intercept.Low + A8MPO + A12TNF |
| 1843 | CAD ~ Intercept.Low + AdipA1 + NTproBNP |
| 1844 | CAD ~ Intercept.Low + NTproBNP + PlGF |
| 1845 | CAD ~ Intercept.Low + AdipA1 + PlGF |
| 1846 | CAD ~ Intercept.Low + AdipA1 + A8MPO |
| 1847 | CAD ~ Intercept.Low + AdipA1 + A12TNF |
| 1848 | CAD ~ Intercept.Low + NTproBNP + A8MPO |
| 1849 | CAD ~ Intercept.Low + NTproBNP + A12TNF |
| 1850 | CAD ~ Intercept.Low + PlGF + A8MPO |
| 1851 | CAD ~ Intercept.Low + PlGF + A12TNF |
| 1852 | CAD ~ Intercept.Low + A8MPO + A12TNF |
| 1853 | CAD ~ Intercept.Low + AdipA1 + NTproBNP |
| 1854 | CAD ~ Intercept.Low + NTproBNP + PlGF |
| 1855 | CAD ~ Intercept.Low + AdipA1 + PlGF |
| 1856 | CAD ~ Intercept.Low + AdipA1 + A8MPO |
| 1857 | CAD ~ Intercept.Low + AdipA1 + A12TNF |
| 1858 | CAD ~ Intercept.Low + NTproBNP + A8MPO |
| 1859 | CAD ~ Intercept.Low + NTproBNP + A12TNF |
| 1860 | CAD ~ Intercept.Low + PlGF + A8MPO |
| 1861 | CAD ~ Intercept.Low + PlGF + A12TNF |
| 1862 | CAD ~ Intercept.Low + AdipA1 + NTproBNP + PlGF |
| 1863 | CAD ~ Intercept.Low + AdipA1 + NTproBNP + A8MPO |
| 1864 | CAD ~ Intercept.Low + AdipA1 + NTproBNP + A12TNF |
| 1865 | CAD ~ Intercept.Low + AdipA1 + PlGF + A8MPO |
| 1866 | CAD ~ Intercept.Low + AdipA1 + PlGF + A12TNF |
| 1867 | CAD ~ Intercept.Low + AdipA1 + A8MPO + A12TNF |
| 1868 | CAD ~ Intercept.Low + NTproBNP + PlGF + A8MPO |
| 1869 | CAD ~ Intercept.Low + NTproBNP + PlGF + A12TNF |
| 1870 | CAD ~ Intercept.Low + NTproBNP + A8MPO + A12TNF |
| 1871 | CAD ~ Intercept.Low + PlGF + A8MPO + A12TNF |
| 1872 | CAD ~ Intercept.Low + A8MPO + A12TNF |
| 1873 | CAD ~ Intercept.Low + AdipA1 + NTproBNP |
| 1874 | CAD ~ Intercept.Low + NTproBNP + PlGF |
| 1875 | CAD ~ Intercept.Low + AdipA1 + PlGF |
| 1876 | CAD ~ Intercept.Low + AdipA1 + A8MPO |
| 1877 | CAD ~ Intercept.Low + AdipA1 + A12TNF |
| 1878 | CAD ~ Intercept.Low + NTproBNP + A8MPO |
| 1879 | CAD ~ Intercept.Low + NTproBNP + A12TNF |
| 1880 | CAD ~ Intercept.Low + PlGF + A8MPO |
| 1881 | CAD ~ Intercept.Low + PlGF + A12TNF |
| 1882 | CAD ~ Intercept.Low + AdipA1 + NTproBNP + PlGF |
| 1883 | CAD ~ Intercept.Low + AdipA1 + NTproBNP + A8MPO |
| 1884 | CAD ~ Intercept.Low + AdipA1 + NTproBNP + A12TNF |
| 1885 | CAD ~ Intercept.Low + AdipA1 + PlGF + A8MPO |
| 1886 | CAD ~ Intercept.Low + AdipA1 + PlGF + A12TNF |
| 1887 | CAD ~ Intercept.Low + AdipA1 + A8MPO + A12TNF |
| 1888 | CAD ~ Intercept.Low + NTproBNP + PlGF + A8MPO |
| 1889 | CAD ~ Intercept.Low + NTproBNP + PlGF + A12TNF |
| 1890 | CAD ~ Intercept.Low + NTproBNP + A8MPO + A12TNF |
| 1891 | CAD ~ Intercept.Low + PlGF + A8MPO + A12TNF |
| 1892 | CAD ~ Intercept.Low + AdipA1 + NTproBNP + PlGF |
| 1893 | CAD ~ Intercept.Low + AdipA1 + NTproBNP + A8MPO |
| 1894 | CAD ~ Intercept.Low + AdipA1 + NTproBNP + A12TNF |
| 1895 | CAD ~ Intercept.Low + AdipA1 + PlGF + A8MPO |
| 1896 | CAD ~ Intercept.Low + AdipA1 + PlGF + A12TNF |
| 1897 | CAD ~ Intercept.Low + AdipA1 + A8MPO + A12TNF |
| 1898 | CAD ~ Intercept.Low + NTproBNP + PlGF + A8MPO |
| 1899 | CAD ~ Intercept.Low + NTproBNP + PlGF + A12TNF |
| 1900 | CAD ~ Intercept.Low + NTproBNP + A8MPO + A12TNF |
| 1901 | CAD ~ Intercept.Low + PlGF + A8MPO + A12TNF |
| 1902 | CAD ~ Intercept.Low + AdipA1 + NTproBNP + PlGF + A8MPO |
| 1903 | CAD ~ Intercept.Low + AdipA1 + NTproBNP + PlGF + A12TNF |
| 1904 | CAD ~ Intercept.Low + AdipA1 + NTproBNP + A8MPO + A12TNF |
| 1905 | CAD ~ Intercept.Low + AdipA1 + PlGF + A8MPO + A12TNF |
| 1906 | CAD ~ Intercept.Low + NTproBNP + PlGF + A8MPO + A12TNF |
| 1907 | CAD ~ Intercept.Low + A8MPO + A12TNF |
| 1908 | CAD ~ Intercept.Low + AdipA1 + NTproBNP |
| 1909 | CAD ~ Intercept.Low + AdipA1 + PlGF |
| 1910 | CAD ~ Intercept.Low + NTproBNP + PlGF |
| 1911 | CAD ~ Intercept.Low + AdipA1 + A8MPO |
| 1912 | CAD ~ Intercept.Low + AdipA1 + A12TNF |
| 1913 | CAD ~ Intercept.Low + NTproBNP + A8MPO |
| 1914 | CAD ~ Intercept.Low + NTproBNP + A12TNF |
| 1915 | CAD ~ Intercept.Low + PlGF + A8MPO |
| 1916 | CAD ~ Intercept.Low + PlGF + A12TNF |
| 1917 | CAD ~ Intercept.Low + AdipA1 + NTproBNP + PlGF |
| 1918 | CAD ~ Intercept.Low + AdipA1 + NTproBNP + A8MPO |
| 1919 | CAD ~ Intercept.Low + AdipA1 + NTproBNP + A12TNF |
| 1920 | CAD ~ Intercept.Low + AdipA1 + PlGF + A8MPO |
| 1921 | CAD ~ Intercept.Low + AdipA1 + PlGF + A12TNF |
| 1922 | CAD ~ Intercept.Low + AdipA1 + A8MPO + A12TNF |
| 1923 | CAD ~ Intercept.Low + NTproBNP + PlGF + A8MPO |
| 1924 | CAD ~ Intercept.Low + NTproBNP + PlGF + A12TNF |
| 1925 | CAD ~ Intercept.Low + NTproBNP + A8MPO + A12TNF |
| 1926 | CAD ~ Intercept.Low + PlGF + A8MPO + A12TNF |
| 1927 | CAD ~ Intercept.Low + AdipA1 + NTproBNP + PlGF |
| 1928 | CAD ~ Intercept.Low + AdipA1 + NTproBNP + A8MPO |
| 1929 | CAD ~ Intercept.Low + AdipA1 + NTproBNP + A12TNF |
| 1930 | CAD ~ Intercept.Low + AdipA1 + PlGF + A8MPO |
| 1931 | CAD ~ Intercept.Low + AdipA1 + PlGF + A12TNF |
| 1932 | CAD ~ Intercept.Low + AdipA1 + A8MPO + A12TNF |
| 1933 | CAD ~ Intercept.Low + NTproBNP + PlGF + A8MPO |
| 1934 | CAD ~ Intercept.Low + NTproBNP + PlGF + A12TNF |
| 1935 | CAD ~ Intercept.Low + NTproBNP + A8MPO + A12TNF |
| 1936 | CAD ~ Intercept.Low + PlGF + A8MPO + A12TNF |
| 1937 | CAD ~ Intercept.Low + AdipA1 + NTproBNP + PlGF + A8MPO |
| 1938 | CAD ~ Intercept.Low + AdipA1 + NTproBNP + PlGF + A12TNF |
| 1939 | CAD ~ Intercept.Low + AdipA1 + NTproBNP + A8MPO + A12TNF |
| 1940 | CAD ~ Intercept.Low + AdipA1 + PlGF + A8MPO + A12TNF |
| 1941 | CAD ~ Intercept.Low + NTproBNP + PlGF + A8MPO + A12TNF |
| 1942 | CAD ~ Intercept.Low + AdipA1 + NTproBNP + PlGF |
| 1943 | CAD ~ Intercept.Low + AdipA1 + NTproBNP + A8MPO |
| 1944 | CAD ~ Intercept.Low + AdipA1 + NTproBNP + A12TNF |
| 1945 | CAD ~ Intercept.Low + AdipA1 + PlGF + A8MPO |
| 1946 | CAD ~ Intercept.Low + AdipA1 + PlGF + A12TNF |
| 1947 | CAD ~ Intercept.Low + AdipA1 + A8MPO + A12TNF |
| 1948 | CAD ~ Intercept.Low + NTproBNP + PlGF + A8MPO |
| 1949 | CAD ~ Intercept.Low + NTproBNP + PlGF + A12TNF |
| 1950 | CAD ~ Intercept.Low + NTproBNP + A8MPO + A12TNF |
| 1951 | CAD ~ Intercept.Low + PlGF + A8MPO + A12TNF |
| 1952 | CAD ~ Intercept.Low + AdipA1 + NTproBNP + PlGF + A8MPO |
| 1953 | CAD ~ Intercept.Low + AdipA1 + NTproBNP + PlGF + A12TNF |
| 1954 | CAD ~ Intercept.Low + AdipA1 + NTproBNP + A8MPO + A12TNF |
| 1955 | CAD ~ Intercept.Low + AdipA1 + PlGF + A8MPO + A12TNF |
| 1956 | CAD ~ Intercept.Low + NTproBNP + PlGF + A8MPO + A12TNF |
| 1957 | CAD ~ Intercept.Low + AdipA1 + NTproBNP + PlGF + A8MPO |
| 1958 | CAD ~ Intercept.Low + AdipA1 + NTproBNP + PlGF + A12TNF |
| 1959 | CAD ~ Intercept.Low + AdipA1 + NTproBNP + A8MPO + A12TNF |
| 1960 | CAD ~ Intercept.Low + AdipA1 + PlGF + A8MPO + A12TNF |
| 1961 | CAD ~ Intercept.Low + NTproBNP + PlGF + A8MPO + A12TNF |
| 1962 | CAD ~ Intercept.Low + AdipA1 + NTproBNP + PlGF + A8MPO + A12TNF |
| 1963 | CAD ~ Intercept.Low + A8MPO + A12TNF |
| 1964 | CAD ~ Intercept.Low + AdipA1 + NTproBNP |
| 1965 | CAD ~ Intercept.Low + AdipA1 + PlGF |
| 1966 | CAD ~ Intercept.Low + NTproBNP + PlGF |
| 1967 | CAD ~ Intercept.Low + AdipA1 + A8MPO |
| 1968 | CAD ~ Intercept.Low + AdipA1 + A12TNF |
| 1969 | CAD ~ Intercept.Low + NTproBNP + A8MPO |
| 1970 | CAD ~ Intercept.Low + NTproBNP + A12TNF |
| 1971 | CAD ~ Intercept.Low + PlGF + A8MPO |
| 1972 | CAD ~ Intercept.Low + PlGF + A12TNF |
| 1973 | CAD ~ Intercept.Low + AdipA1 + NTproBNP + PlGF |
| 1974 | CAD ~ Intercept.Low + AdipA1 + NTproBNP + A8MPO |
| 1975 | CAD ~ Intercept.Low + AdipA1 + NTproBNP + A12TNF |
| 1976 | CAD ~ Intercept.Low + AdipA1 + PlGF + A8MPO |
| 1977 | CAD ~ Intercept.Low + AdipA1 + PlGF + A12TNF |
| 1978 | CAD ~ Intercept.Low + AdipA1 + A8MPO + A12TNF |
| 1979 | CAD ~ Intercept.Low + NTproBNP + PlGF + A8MPO |
| 1980 | CAD ~ Intercept.Low + NTproBNP + PlGF + A12TNF |
| 1981 | CAD ~ Intercept.Low + NTproBNP + A8MPO + A12TNF |
| 1982 | CAD ~ Intercept.Low + PlGF + A8MPO + A12TNF |
| 1983 | CAD ~ Intercept.Low + AdipA1 + NTproBNP + PlGF |
| 1984 | CAD ~ Intercept.Low + AdipA1 + NTproBNP + A8MPO |
| 1985 | CAD ~ Intercept.Low + AdipA1 + NTproBNP + A12TNF |
| 1986 | CAD ~ Intercept.Low + AdipA1 + PlGF + A8MPO |
| 1987 | CAD ~ Intercept.Low + AdipA1 + PlGF + A12TNF |
| 1988 | CAD ~ Intercept.Low + AdipA1 + A8MPO + A12TNF |
| 1989 | CAD ~ Intercept.Low + NTproBNP + PlGF + A8MPO |
| 1990 | CAD ~ Intercept.Low + NTproBNP + PlGF + A12TNF |
| 1991 | CAD ~ Intercept.Low + NTproBNP + A8MPO + A12TNF |
| 1992 | CAD ~ Intercept.Low + PlGF + A8MPO + A12TNF |
| 1993 | CAD ~ Intercept.Low + AdipA1 + NTproBNP + PlGF + A8MPO |
| 1994 | CAD ~ Intercept.Low + AdipA1 + NTproBNP + PlGF + A12TNF |
| 1995 | CAD ~ Intercept.Low + AdipA1 + NTproBNP + A8MPO + A12TNF |
| 1996 | CAD ~ Intercept.Low + AdipA1 + PlGF + A8MPO + A12TNF |
| 1997 | CAD ~ Intercept.Low + NTproBNP + PlGF + A8MPO + A12TNF |
| 1998 | CAD ~ Intercept.Low + AdipA1 + NTproBNP + PlGF |
| 1999 | CAD ~ Intercept.Low + AdipA1 + NTproBNP + A8MPO |
| 2000 | CAD ~ Intercept.Low + AdipA1 + NTproBNP + A12TNF |
| 2001 | CAD ~ Intercept.Low + AdipA1 + PlGF + A8MPO |
| 2002 | CAD ~ Intercept.Low + AdipA1 + PlGF + A12TNF |
| 2003 | CAD ~ Intercept.Low + AdipA1 + A8MPO + A12TNF |
| 2004 | CAD ~ Intercept.Low + NTproBNP + PlGF + A8MPO |
| 2005 | CAD ~ Intercept.Low + NTproBNP + PlGF + A12TNF |
| 2006 | CAD ~ Intercept.Low + NTproBNP + A8MPO + A12TNF |
| 2007 | CAD ~ Intercept.Low + PlGF + A8MPO + A12TNF |
| 2008 | CAD ~ Intercept.Low + AdipA1 + NTproBNP + PlGF + A8MPO |
| 2009 | CAD ~ Intercept.Low + AdipA1 + NTproBNP + PlGF + A12TNF |
| 2010 | CAD ~ Intercept.Low + AdipA1 + NTproBNP + A8MPO + A12TNF |
| 2011 | CAD ~ Intercept.Low + AdipA1 + PlGF + A8MPO + A12TNF |
| 2012 | CAD ~ Intercept.Low + NTproBNP + PlGF + A8MPO + A12TNF |
| 2013 | CAD ~ Intercept.Low + AdipA1 + NTproBNP + PlGF + A8MPO |
| 2014 | CAD ~ Intercept.Low + AdipA1 + NTproBNP + PlGF + A12TNF |
| 2015 | CAD ~ Intercept.Low + AdipA1 + NTproBNP + A8MPO + A12TNF |
| 2016 | CAD ~ Intercept.Low + AdipA1 + PlGF + A8MPO + A12TNF |
| 2017 | CAD ~ Intercept.Low + NTproBNP + PlGF + A8MPO + A12TNF |
| 2018 | CAD ~ Intercept.Low + AdipA1 + NTproBNP + PlGF + A8MPO + A12TNF |
| 2019 | CAD ~ Intercept.Low + NTproBNP |
| 2020 | CAD ~ Intercept.Low + AdipA1 |
| 2021 | CAD ~ Intercept.Low + PlGF |
| 2022 | CAD ~ Intercept.Low + A8MPO |
| 2023 | CAD ~ Intercept.Low + A12TNF |
| 2024 | CAD ~ Intercept.Low + NTproBNP |
| 2025 | CAD ~ Intercept.Low + AdipA1 |
| 2026 | CAD ~ Intercept.Low + PlGF |
| 2027 | CAD ~ Intercept.Low + A8MPO |
| 2028 | CAD ~ Intercept.Low + A12TNF |
| 2029 | CAD ~ Intercept.Low + NTproBNP |
| 2030 | CAD ~ Intercept.Low + AdipA1 |
| 2031 | CAD ~ Intercept.Low + PlGF |
| 2032 | CAD ~ Intercept.Low + A8MPO |
| 2033 | CAD ~ Intercept.Low + A12TNF |
| 2034 | CAD ~ Intercept.Low + NTproBNP + PlGF |
| 2035 | CAD ~ Intercept.Low + AdipA1 + NTproBNP |
| 2036 | CAD ~ Intercept.Low + AdipA1 + PlGF |
| 2037 | CAD ~ Intercept.Low + AdipA1 + A8MPO |
| 2038 | CAD ~ Intercept.Low + AdipA1 + A12TNF |
| 2039 | CAD ~ Intercept.Low + NTproBNP + A8MPO |
| 2040 | CAD ~ Intercept.Low + NTproBNP + A12TNF |
| 2041 | CAD ~ Intercept.Low + PlGF + A8MPO |
| 2042 | CAD ~ Intercept.Low + PlGF + A12TNF |
| 2043 | CAD ~ Intercept.Low + A8MPO + A12TNF |
| 2044 | CAD ~ Intercept.Low + NTproBNP |
| 2045 | CAD ~ Intercept.Low + AdipA1 |
| 2046 | CAD ~ Intercept.Low + PlGF |
| 2047 | CAD ~ Intercept.Low + A8MPO |
| 2048 | CAD ~ Intercept.Low + A12TNF |
| 2049 | CAD ~ Intercept.Low + NTproBNP |
| 2050 | CAD ~ Intercept.Low + AdipA1 |
| 2051 | CAD ~ Intercept.Low + PlGF |
| 2052 | CAD ~ Intercept.Low + A8MPO |
| 2053 | CAD ~ Intercept.Low + A12TNF |
| 2054 | CAD ~ Intercept.Low + NTproBNP + PlGF |
| 2055 | CAD ~ Intercept.Low + AdipA1 + NTproBNP |
| 2056 | CAD ~ Intercept.Low + AdipA1 + PlGF |
| 2057 | CAD ~ Intercept.Low + AdipA1 + A8MPO |
| 2058 | CAD ~ Intercept.Low + AdipA1 + A12TNF |
| 2059 | CAD ~ Intercept.Low + NTproBNP + A8MPO |
| 2060 | CAD ~ Intercept.Low + NTproBNP + A12TNF |
| 2061 | CAD ~ Intercept.Low + PlGF + A8MPO |
| 2062 | CAD ~ Intercept.Low + PlGF + A12TNF |
| 2063 | CAD ~ Intercept.Low + A8MPO + A12TNF |
| 2064 | CAD ~ Intercept.Low + NTproBNP |
| 2065 | CAD ~ Intercept.Low + AdipA1 |
| 2066 | CAD ~ Intercept.Low + PlGF |
| 2067 | CAD ~ Intercept.Low + A8MPO |
| 2068 | CAD ~ Intercept.Low + A12TNF |
| 2069 | CAD ~ Intercept.Low + NTproBNP + PlGF |
| 2070 | CAD ~ Intercept.Low + AdipA1 + NTproBNP |
| 2071 | CAD ~ Intercept.Low + AdipA1 + PlGF |
| 2072 | CAD ~ Intercept.Low + AdipA1 + A8MPO |
| 2073 | CAD ~ Intercept.Low + AdipA1 + A12TNF |
| 2074 | CAD ~ Intercept.Low + NTproBNP + A8MPO |
| 2075 | CAD ~ Intercept.Low + NTproBNP + A12TNF |
| 2076 | CAD ~ Intercept.Low + PlGF + A8MPO |
| 2077 | CAD ~ Intercept.Low + PlGF + A12TNF |
| 2078 | CAD ~ Intercept.Low + A8MPO + A12TNF |
| 2079 | CAD ~ Intercept.Low + NTproBNP + PlGF |
| 2080 | CAD ~ Intercept.Low + AdipA1 + NTproBNP |
| 2081 | CAD ~ Intercept.Low + AdipA1 + PlGF |
| 2082 | CAD ~ Intercept.Low + AdipA1 + A8MPO |
| 2083 | CAD ~ Intercept.Low + AdipA1 + A12TNF |
| 2084 | CAD ~ Intercept.Low + NTproBNP + A8MPO |
| 2085 | CAD ~ Intercept.Low + NTproBNP + A12TNF |
| 2086 | CAD ~ Intercept.Low + PlGF + A8MPO |
| 2087 | CAD ~ Intercept.Low + PlGF + A12TNF |
| 2088 | CAD ~ Intercept.Low + A8MPO + A12TNF |
| 2089 | CAD ~ Intercept.Low + AdipA1 + NTproBNP + PlGF |
| 2090 | CAD ~ Intercept.Low + AdipA1 + NTproBNP + A8MPO |
| 2091 | CAD ~ Intercept.Low + AdipA1 + NTproBNP + A12TNF |
| 2092 | CAD ~ Intercept.Low + AdipA1 + PlGF + A8MPO |
| 2093 | CAD ~ Intercept.Low + AdipA1 + PlGF + A12TNF |
| 2094 | CAD ~ Intercept.Low + AdipA1 + A8MPO + A12TNF |
| 2095 | CAD ~ Intercept.Low + NTproBNP + PlGF + A8MPO |
| 2096 | CAD ~ Intercept.Low + NTproBNP + PlGF + A12TNF |
| 2097 | CAD ~ Intercept.Low + NTproBNP + A8MPO + A12TNF |
| 2098 | CAD ~ Intercept.Low + PlGF + A8MPO + A12TNF |
| 2099 | CAD ~ Intercept.Low + NTproBNP |
| 2100 | CAD ~ Intercept.Low + AdipA1 |
| 2101 | CAD ~ Intercept.Low + PlGF |
| 2102 | CAD ~ Intercept.Low + A8MPO |
| 2103 | CAD ~ Intercept.Low + A12TNF |
| 2104 | CAD ~ Intercept.Low + NTproBNP |
| 2105 | CAD ~ Intercept.Low + AdipA1 |
| 2106 | CAD ~ Intercept.Low + PlGF |
| 2107 | CAD ~ Intercept.Low + A8MPO |
| 2108 | CAD ~ Intercept.Low + A12TNF |
| 2109 | CAD ~ Intercept.Low + NTproBNP + PlGF |
| 2110 | CAD ~ Intercept.Low + AdipA1 + NTproBNP |
| 2111 | CAD ~ Intercept.Low + AdipA1 + PlGF |
| 2112 | CAD ~ Intercept.Low + AdipA1 + A8MPO |
| 2113 | CAD ~ Intercept.Low + AdipA1 + A12TNF |
| 2114 | CAD ~ Intercept.Low + NTproBNP + A8MPO |
| 2115 | CAD ~ Intercept.Low + NTproBNP + A12TNF |
| 2116 | CAD ~ Intercept.Low + PlGF + A8MPO |
| 2117 | CAD ~ Intercept.Low + PlGF + A12TNF |
| 2118 | CAD ~ Intercept.Low + A8MPO + A12TNF |
| 2119 | CAD ~ Intercept.Low + NTproBNP |
| 2120 | CAD ~ Intercept.Low + AdipA1 |
| 2121 | CAD ~ Intercept.Low + PlGF |
| 2122 | CAD ~ Intercept.Low + A8MPO |
| 2123 | CAD ~ Intercept.Low + A12TNF |
| 2124 | CAD ~ Intercept.Low + NTproBNP + PlGF |
| 2125 | CAD ~ Intercept.Low + AdipA1 + NTproBNP |
| 2126 | CAD ~ Intercept.Low + AdipA1 + PlGF |
| 2127 | CAD ~ Intercept.Low + AdipA1 + A8MPO |
| 2128 | CAD ~ Intercept.Low + AdipA1 + A12TNF |
| 2129 | CAD ~ Intercept.Low + NTproBNP + A8MPO |
| 2130 | CAD ~ Intercept.Low + NTproBNP + A12TNF |
| 2131 | CAD ~ Intercept.Low + PlGF + A8MPO |
| 2132 | CAD ~ Intercept.Low + PlGF + A12TNF |
| 2133 | CAD ~ Intercept.Low + A8MPO + A12TNF |
| 2134 | CAD ~ Intercept.Low + NTproBNP + PlGF |
| 2135 | CAD ~ Intercept.Low + AdipA1 + NTproBNP |
| 2136 | CAD ~ Intercept.Low + AdipA1 + PlGF |
| 2137 | CAD ~ Intercept.Low + AdipA1 + A8MPO |
| 2138 | CAD ~ Intercept.Low + AdipA1 + A12TNF |
| 2139 | CAD ~ Intercept.Low + NTproBNP + A8MPO |
| 2140 | CAD ~ Intercept.Low + NTproBNP + A12TNF |
| 2141 | CAD ~ Intercept.Low + PlGF + A8MPO |
| 2142 | CAD ~ Intercept.Low + PlGF + A12TNF |
| 2143 | CAD ~ Intercept.Low + A8MPO + A12TNF |
| 2144 | CAD ~ Intercept.Low + AdipA1 + NTproBNP + PlGF |
| 2145 | CAD ~ Intercept.Low + AdipA1 + NTproBNP + A8MPO |
| 2146 | CAD ~ Intercept.Low + AdipA1 + NTproBNP + A12TNF |
| 2147 | CAD ~ Intercept.Low + AdipA1 + PlGF + A8MPO |
| 2148 | CAD ~ Intercept.Low + AdipA1 + PlGF + A12TNF |
| 2149 | CAD ~ Intercept.Low + AdipA1 + A8MPO + A12TNF |
| 2150 | CAD ~ Intercept.Low + NTproBNP + PlGF + A8MPO |
| 2151 | CAD ~ Intercept.Low + NTproBNP + PlGF + A12TNF |
| 2152 | CAD ~ Intercept.Low + NTproBNP + A8MPO + A12TNF |
| 2153 | CAD ~ Intercept.Low + PlGF + A8MPO + A12TNF |
| 2154 | CAD ~ Intercept.Low + NTproBNP |
| 2155 | CAD ~ Intercept.Low + AdipA1 |
| 2156 | CAD ~ Intercept.Low + PlGF |
| 2157 | CAD ~ Intercept.Low + A8MPO |
| 2158 | CAD ~ Intercept.Low + A12TNF |
| 2159 | CAD ~ Intercept.Low + NTproBNP + PlGF |
| 2160 | CAD ~ Intercept.Low + AdipA1 + NTproBNP |
| 2161 | CAD ~ Intercept.Low + AdipA1 + PlGF |
| 2162 | CAD ~ Intercept.Low + AdipA1 + A8MPO |
| 2163 | CAD ~ Intercept.Low + AdipA1 + A12TNF |
| 2164 | CAD ~ Intercept.Low + NTproBNP + A8MPO |
| 2165 | CAD ~ Intercept.Low + NTproBNP + A12TNF |
| 2166 | CAD ~ Intercept.Low + PlGF + A8MPO |
| 2167 | CAD ~ Intercept.Low + PlGF + A12TNF |
| 2168 | CAD ~ Intercept.Low + A8MPO + A12TNF |
| 2169 | CAD ~ Intercept.Low + NTproBNP + PlGF |
| 2170 | CAD ~ Intercept.Low + AdipA1 + NTproBNP |
| 2171 | CAD ~ Intercept.Low + AdipA1 + PlGF |
| 2172 | CAD ~ Intercept.Low + AdipA1 + A8MPO |
| 2173 | CAD ~ Intercept.Low + AdipA1 + A12TNF |
| 2174 | CAD ~ Intercept.Low + NTproBNP + A8MPO |
| 2175 | CAD ~ Intercept.Low + NTproBNP + A12TNF |
| 2176 | CAD ~ Intercept.Low + PlGF + A8MPO |
| 2177 | CAD ~ Intercept.Low + PlGF + A12TNF |
| 2178 | CAD ~ Intercept.Low + A8MPO + A12TNF |
| 2179 | CAD ~ Intercept.Low + AdipA1 + NTproBNP + PlGF |
| 2180 | CAD ~ Intercept.Low + AdipA1 + NTproBNP + A8MPO |
| 2181 | CAD ~ Intercept.Low + AdipA1 + NTproBNP + A12TNF |
| 2182 | CAD ~ Intercept.Low + AdipA1 + PlGF + A8MPO |
| 2183 | CAD ~ Intercept.Low + AdipA1 + PlGF + A12TNF |
| 2184 | CAD ~ Intercept.Low + AdipA1 + A8MPO + A12TNF |
| 2185 | CAD ~ Intercept.Low + NTproBNP + PlGF + A8MPO |
| 2186 | CAD ~ Intercept.Low + NTproBNP + PlGF + A12TNF |
| 2187 | CAD ~ Intercept.Low + NTproBNP + A8MPO + A12TNF |
| 2188 | CAD ~ Intercept.Low + PlGF + A8MPO + A12TNF |
| 2189 | CAD ~ Intercept.Low + NTproBNP + PlGF |
| 2190 | CAD ~ Intercept.Low + AdipA1 + NTproBNP |
| 2191 | CAD ~ Intercept.Low + AdipA1 + PlGF |
| 2192 | CAD ~ Intercept.Low + AdipA1 + A8MPO |
| 2193 | CAD ~ Intercept.Low + AdipA1 + A12TNF |
| 2194 | CAD ~ Intercept.Low + NTproBNP + A8MPO |
| 2195 | CAD ~ Intercept.Low + NTproBNP + A12TNF |
| 2196 | CAD ~ Intercept.Low + PlGF + A8MPO |
| 2197 | CAD ~ Intercept.Low + PlGF + A12TNF |
| 2198 | CAD ~ Intercept.Low + A8MPO + A12TNF |
| 2199 | CAD ~ Intercept.Low + AdipA1 + NTproBNP + PlGF |
| 2200 | CAD ~ Intercept.Low + AdipA1 + NTproBNP + A8MPO |
| 2201 | CAD ~ Intercept.Low + AdipA1 + NTproBNP + A12TNF |
| 2202 | CAD ~ Intercept.Low + AdipA1 + PlGF + A8MPO |
| 2203 | CAD ~ Intercept.Low + AdipA1 + PlGF + A12TNF |
| 2204 | CAD ~ Intercept.Low + AdipA1 + A8MPO + A12TNF |
| 2205 | CAD ~ Intercept.Low + NTproBNP + PlGF + A8MPO |
| 2206 | CAD ~ Intercept.Low + NTproBNP + PlGF + A12TNF |
| 2207 | CAD ~ Intercept.Low + NTproBNP + A8MPO + A12TNF |
| 2208 | CAD ~ Intercept.Low + PlGF + A8MPO + A12TNF |
| 2209 | CAD ~ Intercept.Low + AdipA1 + NTproBNP + PlGF |
| 2210 | CAD ~ Intercept.Low + AdipA1 + NTproBNP + A8MPO |
| 2211 | CAD ~ Intercept.Low + AdipA1 + NTproBNP + A12TNF |
| 2212 | CAD ~ Intercept.Low + AdipA1 + PlGF + A8MPO |
| 2213 | CAD ~ Intercept.Low + AdipA1 + PlGF + A12TNF |
| 2214 | CAD ~ Intercept.Low + AdipA1 + A8MPO + A12TNF |
| 2215 | CAD ~ Intercept.Low + NTproBNP + PlGF + A8MPO |
| 2216 | CAD ~ Intercept.Low + NTproBNP + PlGF + A12TNF |
| 2217 | CAD ~ Intercept.Low + NTproBNP + A8MPO + A12TNF |
| 2218 | CAD ~ Intercept.Low + PlGF + A8MPO + A12TNF |
| 2219 | CAD ~ Intercept.Low + AdipA1 + NTproBNP + PlGF + A8MPO |
| 2220 | CAD ~ Intercept.Low + AdipA1 + NTproBNP + PlGF + A12TNF |
| 2221 | CAD ~ Intercept.Low + AdipA1 + NTproBNP + A8MPO + A12TNF |
| 2222 | CAD ~ Intercept.Low + AdipA1 + PlGF + A8MPO + A12TNF |
| 2223 | CAD ~ Intercept.Low + NTproBNP + PlGF + A8MPO + A12TNF |
| 2224 | CAD ~ Intercept.Low + NTproBNP |
| 2225 | CAD ~ Intercept.Low + AdipA1 |
| 2226 | CAD ~ Intercept.Low + PlGF |
| 2227 | CAD ~ Intercept.Low + A8MPO |
| 2228 | CAD ~ Intercept.Low + A12TNF |
| 2229 | CAD ~ Intercept.Low + NTproBNP |
| 2230 | CAD ~ Intercept.Low + AdipA1 |
| 2231 | CAD ~ Intercept.Low + PlGF |
| 2232 | CAD ~ Intercept.Low + A8MIPO |
| 2233 | CAD ~ Intercept.Low + A12TNF |
| 2234 | CAD ~ Intercept.Low + NTproBNP + PlGF |
| 2235 | CAD ~ Intercept.Low + AdipA1 + NTproBNP |
| 2236 | CAD ~ Intercept.Low + AdipA1 + PlGF |
| 2237 | CAD ~ Intercept.Low + AdipA1 + A8MPO |
| 2238 | CAD ~ Intercept.Low + AdipA1 + A12TNF |
| 2239 | CAD ~ Intercept.Low + NTproBNP + A8MPO |
| 2240 | CAD ~ Intercept.Low + NTproBNP + A12TNF |
| 2241 | CAD ~ Intercept.Low + PlGF + A8MPO |
| 2242 | CAD ~ Intercept.Low + PlGF + A12TNF |
| 2243 | CAD ~ Intercept.Low + A8MPO + A12TNF |
| 2244 | CAD ~ Intercept.Low + NTproBNP |
| 2245 | CAD ~ Intercept.Low + AdipA1 |
| 2246 | CAD ~ Intercept.Low + PlGF |
| 2247 | CAD ~ Intercept.Low + A8MPO |
| 2248 | CAD ~ Intercept.Low + A12TNF |
| 2249 | CAD ~ Intercept.Low + NTproBNP + PlGF |
| 2250 | CAD ~ Intercept.Low + AdipA1 + NTproBNP |
| 2251 | CAD ~ Intercept.Low + AdipA1 + PlGF |
| 2252 | CAD ~ Intercept.Low + AdipA1 + A8MPO |
| 2253 | CAD ~ Intercept.Low + AdipA1 + A12TNF |
| 2254 | CAD ~ Intercept.Low + NTproBNP + A8MPO |
| 2255 | CAD ~ Intercept.Low + NTproBNP + A12TNF |
| 2256 | CAD ~ Intercept.Low + PlGF + A8MPO |
| 2257 | CAD ~ Intercept.Low + PlGF + A12TNF |
| 2258 | CAD ~ Intercept.Low + A8MPO + A12TNF |
| 2259 | CAD ~ Intercept.Low + NTproBNP + PlGF |
| 2260 | CAD ~ Intercept.Low + AdipA1 + NTproBNP |
| 2261 | CAD ~ Intercept.Low + AdipA1 + PlGF |
| 2262 | CAD ~ Intercept.Low + AdipA1 + A8MPO |
| 2263 | CAD ~ Intercept.Low + AdipA1 + A12TNF |
| 2264 | CAD ~ Intercept.Low + NTproBNP + A8MPO |
| 2265 | CAD ~ Intercept.Low + NTproBNP + A12TNF |
| 2266 | CAD ~ Intercept.Low + PlGF + A8MPO |
| 2267 | CAD ~ Intercept.Low + PlGF + A12TNF |
| 2268 | CAD ~ Intercept.Low + A8MPO + A12TNF |
| 2269 | CAD ~ Intercept.Low + AdipA1 + NTproBNP + PlGF |
| 2270 | CAD ~ Intercept.Low + AdipA1 + NTproBNP + A8MPO |
| 2271 | CAD ~ Intercept.Low + AdipA1 + NTproBNP + A12TNF |
| 2272 | CAD ~ Intercept.Low + AdipA1 + PlGF + A8MPO |
| 2273 | CAD ~ Intercept.Low + AdipA1 + PlGF + A12TNF |
| 2274 | CAD ~ Intercept.Low + AdipA1 + A8MPO + A12TNF |
| 2275 | CAD ~ Intercept.Low + NTproBNP + PlGF + A8MPO |
| 2276 | CAD ~ Intercept.Low + NTproBNP + PlGF + A12TNF |
| 2277 | CAD ~ Intercept.Low + NTproBNP + A8MPO + A12TNF |
| 2278 | CAD ~ Intercept.Low + PlGF + A8MPO + A12TNF |
| 2279 | CAD ~ Intercept.Low + NTproBNP |
| 2280 | CAD ~ Intercept.Low + AdipA1 |
| 2281 | CAD ~ Intercept.Low + PlGF |
| 2282 | CAD ~ Intercept.Low + A8MPO |
| 2283 | CAD ~ Intercept.Low + A12TNF |
| 2284 | CAD ~ Intercept.Low + NTproBNP + PlGF |
| 2285 | CAD ~ Intercept.Low + AdipA1 + NTproBNP |
| 2286 | CAD ~ Intercept.Low + AdipA1 + PlGF |
| 2287 | CAD ~ Intercept.Low + AdipA1 + A8MPO |
| 2288 | CAD ~ Intercept.Low + AdipA1 + A12TNF |
| 2289 | CAD ~ Intercept.Low + NTproBNP + A8MPO |
| 2290 | CAD ~ Intercept.Low + NTproBNP + A12TNF |
| 2291 | CAD ~ Intercept.Low + PlGF + A8MPO |
| 2292 | CAD ~ Intercept.Low + PlGF + A12TNF |
| 2293 | CAD ~ Intercept.Low + A8MPO + A12TNF |
| 2294 | CAD ~ Intercept.Low + NTproBNP + PlGF |
| 2295 | CAD ~ Intercept.Low + AdipA1 + NTproBNP |
| 2296 | CAD ~ Intercept.Low + AdipA1 + PlGF |
| 2297 | CAD ~ Intercept.Low + AdipA1 + A8MPO |
| 2298 | CAD ~ Intercept.Low + AdipA1 + A12TNF |
| 2299 | CAD ~ Intercept.Low + NTproBNP + A8MPO |
| 2300 | CAD ~ Intercept.Low + NTproBNP + A12TNF |
| 2301 | CAD ~ Intercept.Low + PlGF + A8MPO |
| 2302 | CAD ~ Intercept.Low + PlGF + A12TNF |
| 2303 | CAD ~ Intercept.Low + A8MPO + A12TNF |
| 2304 | CAD ~ Intercept.Low + AdipA1 + NTproBNP + PlGF |
| 2305 | CAD ~ Intercept.Low + AdipA1 + NTproBNP + A8MPO |
| 2306 | CAD ~ Intercept.Low + AdipA1 + NTproBNP + A12TNF |
| 2307 | CAD ~ Intercept.Low + AdipA1 + PlGF + A8MPO |
| 2308 | CAD ~ Intercept.Low + AdipA1 + PlGF + A12TNF |
| 2309 | CAD ~ Intercept.Low + AdipA1 + A8MPO + A12TNF |
| 2310 | CAD ~ Intercept.Low + NTproBNP + PlGF + A8MPO |
| 2311 | CAD ~ Intercept.Low + NTproBNP + PlGF + A12TNF |
| 2312 | CAD ~ Intercept.Low + NTproBNP + A8MPO + A12TNF |
| 2313 | CAD ~ Intercept.Low + PlGF + A8MPO + A12TNF |
| 2314 | CAD ~ Intercept.Low + NTproBNP + PlGF |
| 2315 | CAD ~ Intercept.Low + AdipA1 + NTproBNP |
| 2316 | CAD ~ Intercept.Low + AdipA1 + PlGF |
| 2317 | CAD ~ Intercept.Low + AdipA1 + A8MPO |
| 2318 | CAD ~ Intercept.Low + AdipA1 + A12TNF |
| 2319 | CAD ~ Intercept.Low + NTproBNP + A8MPO |
| 2320 | CAD ~ Intercept.Low + NTproBNP + A12TNF |
| 2321 | CAD ~ Intercept.Low + PlGF + A8MPO |
| 2322 | CAD ~ Intercept.Low + PlGF + A12TNF |
| 2323 | CAD ~ Intercept.Low + A8MPO + A12TNF |
| 2324 | CAD ~ Intercept.Low + AdipA1 + NTproBNP + PlGF |
| 2325 | CAD ~ Intercept.Low + AdipA1 + NTproBNP + A8MPO |
| 2326 | CAD ~ Intercept.Low + AdipA1 + NTproBNP + A12TNF |
| 2327 | CAD ~ Intercept.Low + AdipA1 + PlGF + A8MPO |
| 2328 | CAD ~ Intercept.Low + AdipA1 + PlGF + A12TNF |
| 2329 | CAD ~ Intercept.Low + AdipA1 + A8MPO + A12TNF |
| 2330 | CAD ~ Intercept.Low + NTproBNP + PlGF + A8MPO |
| 2331 | CAD ~ Intercept.Low + NTproBNP + PlGF + A12TNF |
| 2332 | CAD ~ Intercept.Low + NTproBNP + A8MPO + A12TNF |
| 2333 | CAD ~ Intercept.Low + PlGF + A8MPO + A12TNF |
| 2334 | CAD ~ Intercept.Low + AdipA1 + NTproBNP + PlGF |
| 2335 | CAD ~ Intercept.Low + AdipA1 + NTproBNP + A8MPO |
| 2336 | CAD ~ Intercept.Low + AdipA1 + NTproBNP + A12TNF |
| 2337 | CAD ~ Intercept.Low + AdipA1 + PlGF + A8MPO |
| 2338 | CAD ~ Intercept.Low + AdipA1 + PlGF + A12TNF |
| 2339 | CAD ~ Intercept.Low + AdipA1 + A8MPO + A12TNF |
| 2340 | CAD ~ Intercept.Low + NTproBNP + PlGF + A8MPO |
| 2341 | CAD ~ Intercept.Low + NTproBNP + PlGF + A12TNF |
| 2342 | CAD ~ Intercept.Low + NTproBNP + A8MPO + A12TNF |
| 2343 | CAD ~ Intercept.Low + PlGF + A8MPO + A12TNF |
| 2344 | CAD ~ Intercept.Low + AdipA1 + NTproBNP + PlGF + A8MPO |
| 2345 | CAD ~ Intercept.Low + AdipA1 + NTproBNP + PlGF + A12TNF |
| 2346 | CAD ~ Intercept.Low + AdipA1 + NTproBNP + A8MPO + A12TNF |
| 2347 | CAD ~ Intercept.Low + AdipA1 + PlGF + A8MPO + A12TNF |
| 2348 | CAD ~ Intercept.Low + NTproBNP + PlGF + A8MPO + A12TNF |
| 2349 | CAD ~ Intercept.Low + NTproBNP |
| 2350 | CAD ~ Intercept.Low + AdipA1 |
| 2351 | CAD ~ Intercept.Low + PlGF |
| 2352 | CAD ~ Intercept.Low + A8MPO |
| 2353 | CAD ~ Intercept.Low + A12TNF |
| 2354 | CAD ~ Intercept.Low + A8MPO + A12TNF |
| 2355 | CAD ~ Intercept.Low + NTproBNP + PlGF |
| 2356 | CAD ~ Intercept.Low + AdipA1 + NTproBNP |
| 2357 | CAD ~ Intercept.Low + AdipA1 + PlGF |
| 2358 | CAD ~ Intercept.Low + AdipA1 + A8MPO |
| 2359 | CAD ~ Intercept.Low + AdipA1 + A12TNF |
| 2360 | CAD ~ Intercept.Low + NTproBNP + A8MPO |
| 2361 | CAD ~ Intercept.Low + NTproBNP + A12TNF |
| 2362 | CAD ~ Intercept.Low + PlGF + A8MPO |
| 2363 | CAD ~ Intercept.Low + PlGF + A12TNF |
| 2364 | CAD ~ Intercept.Low + A8MPO + A12TNF |
| 2365 | CAD ~ Intercept.Low + NTproBNP + PlGF |
| 2366 | CAD ~ Intercept.Low + AdipA1 + NTproBNP |
| 2367 | CAD ~ Intercept.Low + AdipA1 + PlGF |
| 2368 | CAD ~ Intercept.Low + AdipA1 + A8MPO |
| 2369 | CAD ~ Intercept.Low + AdipA1 + A12TNF |
| 2370 | CAD ~ Intercept.Low + NTproBNP + A8MPO |
| 2371 | CAD ~ Intercept.Low + NTproBNP + A12TNF |
| 2372 | CAD ~ Intercept.Low + PlGF + A8MPO |
| 2373 | CAD ~ Intercept.Low + PlGF + A12TNF |
| 2374 | CAD ~ Intercept.Low + AdipA1 + NTproBNP + PlGF |
| 2375 | CAD ~ Intercept.Low + AdipA1 + NTproBNP + A8MPO |
| 2376 | CAD ~ Intercept.Low + AdipA1 + NTproBNP + A12TNF |
| 2377 | CAD ~ Intercept.Low + AdipA1 + PlGF + A8MPO |
| 2378 | CAD ~ Intercept.Low + AdipA1 + PlGF + A12TNF |
| 2379 | CAD ~ Intercept.Low + AdipA1 + A8MPO + A12TNF |
| 2380 | CAD ~ Intercept.Low + NTproBNP + PlGF + A8MPO |
| 2381 | CAD ~ Intercept.Low + NTproBNP + PlGF + A12TNF |
| 2382 | CAD ~ Intercept.Low + NTproBNP + A8MPO + A12TNF |
| 2383 | CAD ~ Intercept.Low + PlGF + A8MPO + A12TNF |
| 2384 | CAD ~ Intercept.Low + A8MPO + A12TNF |
| 2385 | CAD ~ Intercept.Low + AdipA1 + NTproBNP |
| 2386 | CAD ~ Intercept.Low + NTproBNP + PlGF |
| 2387 | CAD ~ Intercept.Low + AdipA1 + PlGF |
| 2388 | CAD ~ Intercept.Low + AdipA1 + A8MPO |
| 2389 | CAD ~ Intercept.Low + AdipA1 + A12TNF |
| 2390 | CAD ~ Intercept.Low + NTproBNP + A8MPO |
| 2391 | CAD ~ Intercept.Low + NTproBNP + A12TNF |
| 2392 | CAD ~ Intercept.Low + PlGF + A8MPO |
| 2393 | CAD ~ Intercept.Low + PlGF + A12TNF |
| 2394 | CAD ~ Intercept.Low + AdipA1 + NTproBNP + PlGF |
| 2395 | CAD ~ Intercept.Low + AdipA1 + NTproBNP + A8MPO |
| 2396 | CAD ~ Intercept.Low + AdipA1 + NTproBNP + A12TNF |
| 2397 | CAD ~ Intercept.Low + AdipA1 + PlGF + A8MPO |
| 2398 | CAD ~ Intercept.Low + AdipA1 + PlGF + A12TNF |
| 2399 | CAD ~ Intercept.Low + AdipA1 + A8MPO + A12TNF |
| 2400 | CAD ~ Intercept.Low + NTproBNP + PlGF + A8MPO |
| 2401 | CAD ~ Intercept.Low + NTproBNP + PlGF + A12TNF |
| 2402 | CAD ~ Intercept.Low + NTproBNP + A8MPO + A12TNF |
| 2403 | CAD ~ Intercept.Low + PlGF + A8MPO + A12TNF |
| 2404 | CAD ~ Intercept.Low + AdipA1 + NTproBNP + PlGF |
| 2405 | CAD ~ Intercept.Low + AdipA1 + NTproBNP + A8MPO |
| 2406 | CAD ~ Intercept.Low + AdipA1 + NTproBNP + A12TNF |
| 2407 | CAD ~ Intercept.Low + AdipA1 + PlGF + A8MPO |
| 2408 | CAD ~ Intercept.Low + AdipA1 + PlGF + A12TNF |
| 2409 | CAD ~ Intercept.Low + AdipA1 + A8MPO + A12TNF |
| 2410 | CAD ~ Intercept.Low + NTproBNP + PlGF + A8MPO |
| 2411 | CAD ~ Intercept.Low + NTproBNP + PlGF + A12TNF |
| 2412 | CAD ~ Intercept.Low + NTproBNP + A8MPO + A12TNF |
| 2413 | CAD ~ Intercept.Low + PlGF + A8MPO + A12TNF |
| 2414 | CAD ~ Intercept.Low + AdipA1 + NTproBNP + PlGF + A8MPO |
| 2415 | CAD ~ Intercept.Low + AdipA1 + NTproBNP + PlGF + A12TNF |
| 2416 | CAD ~ Intercept.Low + AdipA1 + NTproBNP + A8MPO + A12TNF |
| 2417 | CAD ~ Intercept.Low + AdipA1 + PlGF + A8MPO + A12TNF |
| 2418 | CAD ~ Intercept.Low + NTproBNP + PlGF + A8MPO + A12TNF |
| 2419 | CAD ~ Intercept.Low + A8MPO + A12TNF |
| 2420 | CAD ~ Intercept.Low + AdipA1 + NTproBNP |
| 2421 | CAD ~ Intercept.Low + NTproBNP + PlGF |
| 2422 | CAD ~ Intercept.Low + AdipA1 + PlGF |
| 2423 | CAD ~ Intercept.Low + AdipA1 + A8MPO |
| 2424 | CAD ~ Intercept.Low + AdipA1 + A12TNF |
| 2425 | CAD ~ Intercept.Low + NTproBNP + A8MPO |
| 2426 | CAD ~ Intercept.Low + NTproBNP + A12TNF |
| 2427 | CAD ~ Intercept.Low + PlGF + A8MPO |
| 2428 | CAD ~ Intercept.Low + PlGF + A12TNF |
| 2429 | CAD ~ Intercept.Low + AdipA1 + NTproBNP + PlGF |
| 2430 | CAD ~ Intercept.Low + AdipA1 + NTproBNP + A8MPO |
| 2431 | CAD ~ Intercept.Low + AdipA1 + NTproBNP + A12TNF |
| 2432 | CAD ~ Intercept.Low + AdipA1 + PlGF + A8MPO |
| 2433 | CAD ~ Intercept.Low + AdipA1 + PlGF + A12TNF |
| 2434 | CAD ~ Intercept.Low + AdipA1 + A8MPO + A12TNF |
| 2435 | CAD ~ Intercept.Low + NTproBNP + PlGF + A8MPO |
| 2436 | CAD ~ Intercept.Low + NTproBNP + PlGF + A12TNF |
| 2437 | CAD ~ Intercept.Low + NTproBNP + A8MPO + A12TNF |
| 2438 | CAD ~ Intercept.Low + PlGF + A8MPO + A12TNF |
| 2439 | CAD ~ Intercept.Low + AdipA1 + NTproBNP + PlGF |
| 2440 | CAD ~ Intercept.Low + AdipA1 + NTproBNP + A8MPO |
| 2441 | CAD ~ Intercept.Low + AdipA1 + NTproBNP + A12TNF |
| 2442 | CAD ~ Intercept.Low + AdipA1 + PlGF + A8MPO |
| 2443 | CAD ~ Intercept.Low + AdipA1 + PlGF + A12TNF |
| 2444 | CAD ~ Intercept.Low + AdipA1 + A8MPO + A12TNF |
| 2445 | CAD ~ Intercept.Low + NTproBNP + PlGF + A8MPO |
| 2446 | CAD ~ Intercept.Low + NTproBNP + PlGF + A12TNF |
| 2447 | CAD ~ Intercept.Low + NTproBNP + A8MPO + A12TNF |
| 2448 | CAD ~ Intercept.Low + PlGF + A8MPO + A12TNF |
| 2449 | CAD ~ Intercept.Low + AdipA1 + NTproBNP + PlGF + A8MPO |
| 2450 | CAD ~ Intercept.Low + AdipA1 + NTproBNP + PlGF + A12TNF |
| 2451 | CAD ~ Intercept.Low + AdipA1 + NTproBNP + A8MPO + A12TNF |
| 2452 | CAD ~ Intercept.Low + AdipA1 + PlGF + A8MPO + A12TNF |
| 2453 | CAD ~ Intercept.Low + NTproBNP + PlGF + A8MPO + A12TNF |
| 2454 | CAD ~ Intercept.Low + AdipA1 + NTproBNP + PlGF |
| 2455 | CAD ~ Intercept.Low + AdipA1 + NTproBNP + A8MPO |
| 2456 | CAD ~ Intercept.Low + AdipA1 + NTproBNP + A12TNF |
| 2457 | CAD ~ Intercept.Low + AdipA1 + PlGF + A8MPO |
| 2458 | CAD ~ Intercept.Low + AdipA1 + PlGF + A12TNF |
| 2459 | CAD ~ Intercept.Low + AdipA1 + A8MPO + A12TNF |
| 2460 | CAD ~ Intercept.Low + NTproBNP + PlGF + A8MPO |
| 2461 | CAD ~ Intercept.Low + NTproBNP + PlGF + A12TNF |
| 2462 | CAD ~ Intercept.Low + NTproBNP + A8MPO + A12TNF |
| 2463 | CAD ~ Intercept.Low + PlGF + A8MPO + A12TNF |
| 2464 | CAD ~ Intercept.Low + AdipA1 + NTproBNP + PlGF + A8MPO |
| 2465 | CAD ~ Intercept.Low + AdipA1 + NTproBNP + PlGF + A12TNF |
| 2466 | CAD ~ Intercept.Low + AdipA1 + NTproBNP + A8MPO + A12TNF |
| 2467 | CAD ~ Intercept.Low + AdipA1 + PlGF + A8MPO + A12TNF |
| 2468 | CAD ~ Intercept.Low + NTproBNP + PlGF + A8MPO + A12TNF |
| 2469 | CAD ~ Intercept.Low + AdipA1 + NTproBNP + PlGF + A8MPO |
| 2470 | CAD ~ Intercept.Low + AdipA1 + NTproBNP + PlGF + A12TNF |
| 2471 | CAD ~ Intercept.Low + AdipA1 + NTproBNP + A8MPO + A12TNF |
| 2472 | CAD ~ Intercept.Low + AdipA1 + PlGF + A8MPO + A12TNF |
| 2473 | CAD ~ Intercept.Low + NTproBNP + PlGF + A8MPO + A12TNF |
| 2474 | CAD ~ Intercept.Low + AdipA1 + NTproBNP + PlGF + A8MPO + A12TNF |
| 2475 | CAD ~ Intercept.Low + AdipA1 + NTproBNP + PlGF |
| 2476 | CAD ~ Intercept.Low + AdipA1 + NTproBNP + A8MPO |
| 2477 | CAD ~ Intercept.Low + AdipA1 + NTproBNP + A12TNF |
| 2478 | CAD ~ Intercept.Low + AdipA1 + PlGF + A8MPO |
| 2479 | CAD ~ Intercept.Low + AdipA1 + PlGF + A12TNF |
| 2480 | CAD ~ Intercept.Low + AdipA1 + A8MPO + A12TNF |
| 2481 | CAD ~ Intercept.Low + NTproBNP + PlGF + A8MPO |
| 2482 | CAD ~ Intercept.Low + NTproBNP + PlGF + A12TNF |
| 2483 | CAD ~ Intercept.Low + NTproBNP + A8MPO + A12TNF |
| 2484 | CAD ~ Intercept.Low + PlGF + A8MPO + A12TNF |
| 2485 | CAD ~ Intercept.Low + AdipA1 + NTproBNP + PlGF + A8MPO |
| 2486 | CAD ~ Intercept.Low + AdipA1 + NTproBNP + PlGF + A12TNF |
| 2487 | CAD ~ Intercept.Low + AdipA1 + NTproBNP + A8MPO + A12TNF |
| 2488 | CAD ~ Intercept.Low + AdipA1 + PlGF + A8MPO + A12TNF |
| 2489 | CAD ~ Intercept.Low + NTproBNP + PlGF + A8MPO + A12TNF |
| 2490 | CAD ~ Intercept.Low + AdipA1 + NTproBNP + PlGF + A8MPO |
| 2491 | CAD ~ Intercept.Low + AdipA1 + NTproBNP + PlGF + A12TNF |
| 2492 | CAD ~ Intercept.Low + AdipA1 + NTproBNP + A8MPO + A12TNF |
| 2493 | CAD ~ Intercept.Low + AdipA1 + PlGF + A8MPO + A12TNF |
| 2494 | CAD ~ Intercept.Low + NTproBNP + PlGF + A8MPO + A12TNF |
| 2495 | CAD ~ Intercept.Low + AdipA1 + NTproBNP + PlGF + A8MPO + A12TNF |
| 2496 | CAD ~ Intercept.Low + AdipA1 + NTproBNP + PlGF + A8MPO |
| 2497 | CAD ~ Intercept.Low + AdipA1 + NTproBNP + PlGF + A12TNF |
| 2498 | CAD ~ Intercept.Low + AdipA1 + NTproBNP + A8MPO + A12TNF |
| 2499 | CAD ~ Intercept.Low + AdipA1 + PlGF + A8MPO + A12TNF |
| 2500 | CAD ~ Intercept.Low + NTproBNP + PlGF + A8MPO + A12TNF |
| 2501 | CAD ~ Intercept.Low + AdipA1 + NTproBNP + PlGF + A8MPO + A12TNF |
| 2502 | CAD ~ Intercept.Low + AdipA1 + NTproBNP + PlGF + A8MPO + A12TNF |
| 2503 | CAD ~ Intercept.Low + NTproBNP |
| 2504 | CAD ~ Intercept.Low + AdipA1 |
| 2505 | CAD ~ Intercept.Low + PlGF |
| 2506 | CAD ~ Intercept.Low + A8MPO |
| 2507 | CAD ~ Intercept.Low + A12TNF |
| 2508 | CAD ~ Intercept.Low + NTproBNP |
| 2509 | CAD ~ Intercept.Low + AdipA1 |
| 2510 | CAD ~ Intercept.Low + PlGF |
| 2511 | CAD ~ Intercept.Low + A8MPO |
| 2512 | CAD ~ Intercept.Low + A12TNF |
| 2513 | CAD ~ Intercept.Low + NTproBNP |
| 2514 | CAD ~ Intercept.Low + AdipA1 |
| 2515 | CAD ~ Intercept.Low + PlGF |
| 2516 | CAD ~ Intercept.Low + A8MPO |
| 2517 | CAD ~ Intercept.Low + A12TNF |
| 2518 | CAD ~ Intercept.Low + NTproBNP + PlGF |
| 2519 | CAD ~ Intercept.Low + AdipA1 + NTproBNP |
| 2520 | CAD ~ Intercept.Low + AdipA1 + PlGF |
| 2521 | CAD ~ Intercept.Low + AdipA1 + A8MPO |
| 2522 | CAD ~ Intercept.Low + AdipA1 + A12TNF |
| 2523 | CAD ~ Intercept.Low + NTproBNP + A8MPO |
| 2524 | CAD ~ Intercept.Low + NTproBNP + A12TNF |
| 2525 | CAD ~ Intercept.Low + PlGF + A8MPO |
| 2526 | CAD ~ Intercept.Low + PlGF + A12TNF |
| 2527 | CAD ~ Intercept.Low + A8MPO + A12TNF |
| 2528 | CAD ~ Intercept.Low + NTproBNP |
| 2529 | CAD ~ Intercept.Low + AdipA1 |
| 2530 | CAD ~ Intercept.Low + PlGF |
| 2531 | CAD ~ Intercept.Low + A8MPO |
| 2532 | CAD ~ Intercept.Low + A12TNF |
| 2533 | CAD ~ Intercept.Low + NTproBNP |
| 2534 | CAD ~ Intercept.Low + AdipA1 |
| 2535 | CAD ~ Intercept.Low + PlGF |
| 2536 | CAD ~ Intercept.Low + A8MPO |
| 2537 | CAD ~ Intercept.Low + A12TNF |
| 2538 | CAD ~ Intercept.Low + NTproBNP + PlGF |
| 2539 | CAD ~ Intercept.Low + AdipA1 + NTproBNP |
| 2540 | CAD ~ Intercept.Low + AdipA1 + PlGF |
| 2541 | CAD ~ Intercept.Low + AdipA1 + A8MPO |
| 2542 | CAD ~ Intercept.Low + AdipA1 + A12TNF |
| 2543 | CAD ~ Intercept.Low + NTproBNP + A8MPO |
| 2544 | CAD ~ Intercept.Low + NTproBNP + A12TNF |
| 2545 | CAD ~ Intercept.Low + PlGF + A8MPO |
| 2546 | CAD ~ Intercept.Low + PlGF + A12TNF |
| 2547 | CAD ~ Intercept.Low + A8MPO + A12TNF |
| 2548 | CAD ~ Intercept.Low + NTproBNP |
| 2549 | CAD ~ Intercept.Low + AdipA1 |
| 2550 | CAD ~ Intercept.Low + PlGF |
| 2551 | CAD ~ Intercept.Low + A8MPO |
| 2552 | CAD ~ Intercept.Low + A12TNF |
| 2553 | CAD ~ Intercept.Low + NTproBNP + PlGF |
| 2554 | CAD ~ Intercept.Low + AdipA1 + NTproBNP |
| 2555 | CAD ~ Intercept.Low + AdipA1 + PlGF |
| 2556 | CAD ~ Intercept.Low + AdipA1 + A8MPO |
| 2557 | CAD ~ Intercept.Low + AdipA1 + A12TNF |
| 2558 | CAD ~ Intercept.Low + NTproBNP + A8MPO |
| 2559 | CAD ~ Intercept.Low + NTproBNP + A12TNF |
| 2560 | CAD ~ Intercept.Low + PlGF + A8MPO |
| 2561 | CAD ~ Intercept.Low + PlGF + A12TNF |
| 2562 | CAD ~ Intercept.Low + A8MPO + A12TNF |
| 2563 | CAD ~ Intercept.Low + NTproBNP + PlGF |
| 2564 | CAD ~ Intercept.Low + AdipA1 + NTproBNP |
| 2565 | CAD ~ Intercept.Low + AdipA1 + PlGF |
| 2566 | CAD ~ Intercept.Low + AdipA1 + A8MPO |
| 2567 | CAD ~ Intercept.Low + AdipA1 + A12TNF |
| 2568 | CAD ~ Intercept.Low + NTproBNP + A8MPO |
| 2569 | CAD ~ Intercept.Low + NTproBNP + A12TNF |
| 2570 | CAD ~ Intercept.Low + PlGF + A8MPO |
| 2571 | CAD ~ Intercept.Low + PlGF + A12TNF |
| 2572 | CAD ~ Intercept.Low + A8MPO + A12TNF |
| 2573 | CAD ~ Intercept.Low + AdipA1 + NTproBNP + PlGF |
| 2574 | CAD ~ Intercept.Low + AdipA1 + NTproBNP + A8MPO |
| 2575 | CAD ~ Intercept.Low + AdipA1 + NTproBNP + A12TNF |
| 2576 | CAD ~ Intercept.Low + AdipA1 + PlGF + A8MPO |
| 2577 | CAD ~ Intercept.Low + AdipA1 + PlGF + A12TNF |
| 2578 | CAD ~ Intercept.Low + AdipA1 + A8MPO + A12TNF |
| 2579 | CAD ~ Intercept.Low + NTproBNP + PlGF + A8MPO |
| 2580 | CAD ~ Intercept.Low + NTproBNP + PlGF + A12TNF |
| 2581 | CAD ~ Intercept.Low + NTproBNP + A8MPO + A12TNF |
| 2582 | CAD ~ Intercept.Low + PlGF + A8MPO + A12TNF |
| 2583 | CAD ~ Intercept.Low + NTproBNP |
| 2584 | CAD ~ Intercept.Low + AdipA1 |
| 2585 | CAD ~ Intercept.Low + PlGF |
| 2586 | CAD ~ Intercept.Low + A8MPO |
| 2587 | CAD ~ Intercept.Low + A12TNF |
| 2588 | CAD ~ Intercept.Low + NTproBNP |
| 2589 | CAD ~ Intercept.Low + AdipA1 |
| 2590 | CAD ~ Intercept.Low + PlGF |
| 2591 | CAD ~ Intercept.Low + A8MPO |
| 2592 | CAD ~ Intercept.Low + A12TNF |
| 2593 | CAD ~ Intercept.Low + NTproBNP + PlGF |
| 2594 | CAD ~ Intercept.Low + AdipA1 + NTproBNP |
| 2595 | CAD ~ Intercept.Low + AdipA1 + PlGF |
| 2596 | CAD ~ Intercept.Low + AdipA1 + A8MPO |
| 2597 | CAD ~ Intercept.Low + AdipA1 + A12TNF |
| 2598 | CAD ~ Intercept.Low + NTproBNP + A8MPO |
| 2599 | CAD ~ Intercept.Low + NTproBNP + A12TNF |
| 2600 | CAD ~ Intercept.Low + PlGF + A8MPO |
| 2601 | CAD ~ Intercept.Low + PlGF + A12TNF |
| 2602 | CAD ~ Intercept.Low + A8MPO + A12TNF |
| 2603 | CAD ~ Intercept.Low + NTproBNP |
| 2604 | CAD ~ Intercept.Low + AdipA1 |
| 2605 | CAD ~ Intercept.Low + PlGF |
| 2606 | CAD ~ Intercept.Low + A8MPO |
| 2607 | CAD ~ Intercept.Low + A12TNF |
| 2608 | CAD ~ Intercept.Low + NTproBNP + PlGF |
| 2609 | CAD ~ Intercept.Low + AdipA1 + NTproBNP |
| 2610 | CAD ~ Intercept.Low + AdipA1 + PlGF |
| 2611 | CAD ~ Intercept.Low + AdipA1 + A8MPO |
| 2612 | CAD ~ Intercept.Low + AdipA1 + A12TNF |
| 2613 | CAD ~ Intercept.Low + NTproBNP + A8MPO |
| 2614 | CAD ~ Intercept.Low + NTproBNP + A12TNF |
| 2615 | CAD ~ Intercept.Low + PlGF + A8MPO |
| 2616 | CAD ~ Intercept.Low + PlGF + A12TNF |
| 2617 | CAD ~ Intercept.Low + A8MPO + A12TNF |
| 2618 | CAD ~ Intercept.Low + NTproBNP + PlGF |
| 2619 | CAD ~ Intercept.Low + AdipA1 + NTproBNP |
| 2620 | CAD ~ Intercept.Low + AdipA1 + PlGF |
| 2621 | CAD ~ Intercept.Low + AdipA1 + A8MPO |
| 2622 | CAD ~ Intercept.Low + AdipA1 + A12TNF |
| 2623 | CAD ~ Intercept.Low + NTproBNP + A8MPO |
| 2624 | CAD ~ Intercept.Low + NTproBNP + A12TNF |
| 2625 | CAD ~ Intercept.Low + PlGF + A8MPO |
| 2626 | CAD ~ Intercept.Low + PlGF + A12TNF |
| 2627 | CAD ~ Intercept.Low + A8MPO + A12TNF |
| 2628 | CAD ~ Intercept.Low + AdipA1 + NTproBNP + PlGF |
| 2629 | CAD ~ Intercept.Low + AdipA1 + NTproBNP + A8MPO |
| 2630 | CAD ~ Intercept.Low + AdipA1 + NTproBNP + A12TNF |
| 2631 | CAD ~ Intercept.Low + AdipA1 + PlGF + A8MPO |
| 2632 | CAD ~ Intercept.Low + AdipA1 + PlGF + A12TNF |
| 2633 | CAD ~ Intercept.Low + AdipA1 + A8MPO + A12TNF |
| 2634 | CAD ~ Intercept.Low + NTproBNP + PlGF + A8MPO |
| 2635 | CAD ~ Intercept.Low + NTproBNP + PlGF + A12TNF |
| 2636 | CAD ~ Intercept.Low + NTproBNP + A8MPO + A12TNF |
| 2637 | CAD ~ Intercept.Low + PlGF + A8MPO + A12TNF |
| 2638 | CAD ~ Intercept.Low + NTproBNP |
| 2639 | CAD ~ Intercept.Low + AdipA1 |
| 2640 | CAD ~ Intercept.Low + PlGF |
| 2641 | CAD ~ Intercept.Low + A8MPO |
| 2642 | CAD ~ Intercept.Low + A12TNF |
| 2643 | CAD ~ Intercept.Low + NTproBNP + PlGF |
| 2644 | CAD ~ Intercept.Low + AdipA1 + NTproBNP |
| 2645 | CAD ~ Intercept.Low + AdipA1 + PlGF |
| 2646 | CAD ~ Intercept.Low + AdipA1 + A8MPO |
| 2647 | CAD ~ Intercept.Low + AdipA1 + A12TNF |
| 2648 | CAD ~ Intercept.Low + NTproBNP + A8MPO |
| 2649 | CAD ~ Intercept.Low + NTproBNP + A12TNF |
| 2650 | CAD ~ Intercept.Low + PlGF + A8MPO |
| 2651 | CAD ~ Intercept.Low + PlGF + A12TNF |
| 2652 | CAD ~ Intercept.Low + A8MPO + A12TNF |
| 2653 | CAD ~ Intercept.Low + NTproBNP + PlGF |
| 2654 | CAD ~ Intercept.Low + AdipA1 + NTproBNP |
| 2655 | CAD ~ Intercept.Low + AdipA1 + PlGF |
| 2656 | CAD ~ Intercept.Low + AdipA1 + A8MPO |
| 2657 | CAD ~ Intercept.Low + AdipA1 + A12TNF |
| 2658 | CAD ~ Intercept.Low + NTproBNP + A8MPO |
| 2659 | CAD ~ Intercept.Low + NTproBNP + A12TNF |
| 2660 | CAD ~ Intercept.Low + PlGF + A8MPO |
| 2661 | CAD ~ Intercept.Low + PlGF + A12TNF |
| 2662 | CAD ~ Intercept.Low + A8MPO + A12TNF |
| 2663 | CAD ~ Intercept.Low + AdipA1 + NTproBNP + PlGF |
| 2664 | CAD ~ Intercept.Low + AdipA1 + NTproBNP + A8MPO |
| 2665 | CAD ~ Intercept.Low + AdipA1 + NTproBNP + A12TNF |
| 2666 | CAD ~ Intercept.Low + AdipA1 + PlGF + A8MPO |
| 2667 | CAD ~ Intercept.Low + AdipA1 + PlGF + A12TNF |
| 2668 | CAD ~ Intercept.Low + AdipA1 + A8MPO + A12TNF |
| 2669 | CAD ~ Intercept.Low + NTproBNP + PlGF + A8MPO |
| 2670 | CAD ~ Intercept.Low + NTproBNP + PlGF + A12TNF |
| 2671 | CAD ~ Intercept.Low + NTproBNP + A8MPO + A12TNF |
| 2672 | CAD ~ Intercept.Low + PlGF + A8MPO + A12TNF |
| 2673 | CAD ~ Intercept.Low + NTproBNP + PlGF |
| 2674 | CAD ~ Intercept.Low + AdipA1 + NTproBNP |
| 2675 | CAD ~ Intercept.Low + AdipA1 + PlGF |
| 2676 | CAD ~ Intercept.Low + AdipA1 + A8MPO |
| 2677 | CAD ~ Intercept.Low + AdipA1 + A12TNF |
| 2678 | CAD ~ Intercept.Low + NTproBNP + A8MPO |
| 2679 | CAD ~ Intercept.Low + NTproBNP + A12TNF |
| 2680 | CAD ~ Intercept.Low + PlGF + A8MPO |
| 2681 | CAD ~ Intercept.Low + PlGF + A12TNF |
| 2682 | CAD ~ Intercept.Low + A8MPO + A12TNF |
| 2683 | CAD ~ Intercept.Low + AdipA1 + NTproBNP + PlGF |
| 2684 | CAD ~ Intercept.Low + AdipA1 + NTproBNP + A8MPO |
| 2685 | CAD ~ Intercept.Low + AdipA1 + NTproBNP + A12TNF |
| 2686 | CAD ~ Intercept.Low + AdipA1 + PlGF + A8MPO |
| 2687 | CAD ~ Intercept.Low + AdipA1 + PlGF + A12TNF |
| 2688 | CAD ~ Intercept.Low + AdipA1 + A8MPO + A12TNF |
| 2689 | CAD ~ Intercept.Low + NTproBNP + PlGF + A8MPO |
| 2690 | CAD ~ Intercept.Low + NTproBNP + PlGF + A12TNF |
| 2691 | CAD ~ Intercept.Low + NTproBNP + A8MPO + A12TNF |
| 2692 | CAD ~ Intercept.Low + PlGF + A8MPO + A12TNF |
| 2693 | CAD ~ Intercept.Low + AdipA1 + NTproBNP + PlGF |
| 2694 | CAD ~ Intercept.Low + AdipA1 + NTproBNP + A8MPO |
| 2695 | CAD ~ Intercept.Low + AdipA1 + NTproBNP + A12TNF |
| 2696 | CAD ~ Intercept.Low + AdipA1 + PlGF + A8MPO |
| 2697 | CAD ~ Intercept.Low + AdipA1 + PlGF + A12TNF |
| 2698 | CAD ~ Intercept.Low + AdipA1 + A8MPO + A12TNF |
| 2699 | CAD ~ Intercept.Low + NTproBNP + PlGF + A8MPO |
| 2700 | CAD ~ Intercept.Low + NTproBNP + PlGF + A12TNF |
| 2701 | CAD ~ Intercept.Low + NTproBNP + A8MPO + A12TNF |
| 2702 | CAD ~ Intercept.Low + PlGF + A8MPO + A12TNF |
| 2703 | CAD ~ Intercept.Low + AdipA1 + NTproBNP + PlGF + A8MPO |
| 2704 | CAD ~ Intercept.Low + AdipA1 + NTproBNP + PlGF + A12TNF |
| 2705 | CAD ~ Intercept.Low + AdipA1 + NTproBNP + A8MPO + A12TNF |
| 2706 | CAD ~ Intercept.Low + AdipA1 + PlGF + A8MPO + A12TNF |
| 2707 | CAD ~ Intercept.Low + NTproBNP + PlGF + A8MPO + A12TNF |
| 2708 | CAD ~ Intercept.Low + NTproBNP |
| 2709 | CAD ~ Intercept.Low + AdipA1 |
| 2710 | CAD ~ Intercept.Low + PlGF |
| 2711 | CAD ~ Intercept.Low + A8MPO |
| 2712 | CAD ~ Intercept.Low + A12TNF |
| 2713 | CAD ~ Intercept.Low + NTproBNP |
| 2714 | CAD ~ Intercept.Low + AdipA1 |
| 2715 | CAD ~ Intercept.Low + PlGF |
| 2716 | CAD ~ Intercept.Low + A8MPO |
| 2717 | CAD ~ Intercept.Low + A12TNF |
| 2718 | CAD ~ Intercept.Low + NTproBNP + PlGF |
| 2719 | CAD ~ Intercept.Low + AdipA1 + NTproBNP |
| 2720 | CAD ~ Intercept.Low + AdipA1 + PlGF |
| 2721 | CAD ~ Intercept.Low + AdipA1 + A8MPO |
| 2722 | CAD ~ Intercept.Low + AdipA1 + A12TNF |
| 2723 | CAD ~ Intercept.Low + NTproBNP + A8MPO |
| 2724 | CAD ~ Intercept.Low + NTproBNP + A12TNF |
| 2725 | CAD ~ Intercept.Low + PlGF + A8MPO |
| 2726 | CAD ~ Intercept.Low + PlGF + A12TNF |
| 2727 | CAD ~ Intercept.Low + A8MPO + A12TNF |
| 2728 | CAD ~ Intercept.Low + NTproBNP |
| 2729 | CAD ~ Intercept.Low + AdipA1 |
| 2730 | CAD ~ Intercept.Low + PlGF |
| 2731 | CAD ~ Intercept.Low + A8MPO |
| 2732 | CAD ~ Intercept.Low + A12TNF |
| 2733 | CAD ~ Intercept.Low + NTproBNP + PlGF |
| 2734 | CAD ~ Intercept.Low + AdipA1 + NTproBNP |
| 2735 | CAD ~ Intercept.Low + AdipA1 + PlGF |
| 2736 | CAD ~ Intercept.Low + AdipA1 + A8MPO |
| 2737 | CAD ~ Intercept.Low + AdipA1 + A12TNF |
| 2738 | CAD ~ Intercept.Low + NTproBNP + A8MPO |
| 2739 | CAD ~ Intercept.Low + NTproBNP + A12TNF |
| 2740 | CAD ~ Intercept.Low + PlGF + A8MPO |
| 2741 | CAD ~ Intercept.Low + PlGF + A12TNF |
| 2742 | CAD ~ Intercept.Low + A8MPO + A12TNF |
| 2743 | CAD ~ Intercept.Low + NTproBNP + PlGF |
| 2744 | CAD ~ Intercept.Low + AdipA1 + NTproBNP |
| 2745 | CAD ~ Intercept.Low + AdipA1 + PlGF |
| 2746 | CAD ~ Intercept.Low + AdipA1 + A8MPO |
| 2747 | CAD ~ Intercept.Low + AdipA1 + A12TNF |
| 2748 | CAD ~ Intercept.Low + NTproBNP + A8MPO |
| 2749 | CAD ~ Intercept.Low + NTproBNP + A12TNF |
| 2750 | CAD ~ Intercept.Low + PlGF + A8MPO |
| 2751 | CAD ~ Intercept.Low + PlGF + A12TNF |
| 2752 | CAD ~ Intercept.Low + A8MPO + A12TNF |
| 2753 | CAD ~ Intercept.Low + AdipA1 + NTproBNP + PlGF |
| 2754 | CAD ~ Intercept.Low + AdipA1 + NTproBNP + A8MPO |
| 2755 | CAD ~ Intercept.Low + AdipA1 + NTproBNP + A12TNF |
| 2756 | CAD ~ Intercept.Low + AdipA1 + PlGF + A8MPO |
| 2757 | CAD ~ Intercept.Low + AdipA1 + PlGF + A12TNF |
| 2758 | CAD ~ Intercept.Low + AdipA1 + A8MPO + A12TNF |
| 2759 | CAD ~ Intercept.Low + NTproBNP + PlGF + A8MPO |
| 2760 | CAD ~ Intercept.Low + NTproBNP + PlGF + A12TNF |
| 2761 | CAD ~ Intercept.Low + NTproBNP + A8MPO + A12TNF |
| 2762 | CAD ~ Intercept.Low + PlGF + A8MPO + A12TNF |
| 2763 | CAD ~ Intercept.Low + NTproBNP |
| 2764 | CAD ~ Intercept.Low + AdipA1 |
| 2765 | CAD ~ Intercept.Low + PlGF |
| 2766 | CAD ~ Intercept.Low + A8MPO |
| 2767 | CAD ~ Intercept.Low + A12TNF |
| 2768 | CAD ~ Intercept.Low + NTproBNP + PlGF |
| 2769 | CAD ~ Intercept.Low + AdipA1 + NTproBNP |
| 2770 | CAD ~ Intercept.Low + AdipA1 + PlGF |
| 2771 | CAD ~ Intercept.Low + AdipA1 + A8MPO |
| 2772 | CAD ~ Intercept.Low + AdipA1 + A12TNF |
| 2773 | CAD ~ Intercept.Low + NTproBNP + A8MPO |
| 2774 | CAD ~ Intercept.Low + NTproBNP + A12TNF |
| 2775 | CAD ~ Intercept.Low + PlGF + A8MPO |
| 2776 | CAD ~ Intercept.Low + PlGF + A12TNF |
| 2777 | CAD ~ Intercept.Low + A8MPO + A12TNF |
| 2778 | CAD ~ Intercept.Low + NTproBNP + PlGF |
| 2779 | CAD ~ Intercept.Low + AdipA1 + NTproBNP |
| 2780 | CAD ~ Intercept.Low + AdipA1 + PlGF |
| 2781 | CAD ~ Intercept.Low + AdipA1 + A8MPO |
| 2782 | CAD ~ Intercept.Low + AdipA1 + A12TNF |
| 2783 | CAD ~ Intercept.Low + NTproBNP + A8MPO |
| 2784 | CAD ~ Intercept.Low + NTproBNP + A12TNF |
| 2785 | CAD ~ Intercept.Low + PlGF + A8MPO |
| 2786 | CAD ~ Intercept.Low + PlGF + A12TNF |
| 2787 | CAD ~ Intercept.Low + A8MPO + A12TNF |
| 2788 | CAD ~ Intercept.Low + AdipA1 + NTproBNP + PlGF |
| 2789 | CAD ~ Intercept.Low + AdipA1 + NTproBNP + A8MPO |
| 2790 | CAD ~ Intercept.Low + AdipA1 + NTproBNP + A12TNF |
| 2791 | CAD ~ Intercept.Low + AdipA1 + PlGF + A8MPO |
| 2792 | CAD ~ Intercept.Low + AdipA1 + PlGF + A12TNF |
| 2793 | CAD ~ Intercept.Low + AdipA1 + A8MPO + A12TNF |
| 2794 | CAD ~ Intercept.Low + NTproBNP + PlGF + A8MPO |
| 2795 | CAD ~ Intercept.Low + NTproBNP + PlGF + A12TNF |
| 2796 | CAD ~ Intercept.Low + NTproBNP + A8MPO + A12TNF |
| 2797 | CAD ~ Intercept.Low + PlGF + A8MPO + A12TNF |
| 2798 | CAD ~ Intercept.Low + NTproBNP + PlGF |
| 2799 | CAD ~ Intercept.Low + AdipA1 + NTproBNP |
| 2800 | CAD ~ Intercept.Low + AdipA1 + PlGF |
| 2801 | CAD ~ Intercept.Low + AdipA1 + A8MPO |
| 2802 | CAD ~ Intercept.Low + AdipA1 + A12TNF |
| 2803 | CAD ~ Intercept.Low + NTproBNP + A8MPO |
| 2804 | CAD ~ Intercept.Low + NTproBNP + A12TNF |
| 2805 | CAD ~ Intercept.Low + PlGF + A8MPO |
| 2806 | CAD ~ Intercept.Low + PlGF + A12TNF |
| 2807 | CAD ~ Intercept.Low + A8MPO + A12TNF |
| 2808 | CAD ~ Intercept.Low + AdipA1 + NTproBNP + PlGF |
| 2809 | CAD ~ Intercept.Low + AdipA1 + NTproBNP + A8MPO |
| 2810 | CAD ~ Intercept.Low + AdipA1 + NTproBNP + A12TNF |
| 2811 | CAD ~ Intercept.Low + AdipA1 + PlGF + A8MPO |
| 2812 | CAD ~ Intercept.Low + AdipA1 + PlGF + A12TNF |
| 2813 | CAD ~ Intercept.Low + AdipA1 + A8MPO + A12TNF |
| 2814 | CAD ~ Intercept.Low + NTproBNP + PlGF + A8MPO |
| 2815 | CAD ~ Intercept.Low + NTproBNP + PlGF + A12TNF |
| 2816 | CAD ~ Intercept.Low + NTproBNP + A8MPO + A12TNF |
| 2817 | CAD ~ Intercept.Low + PlGF + A8MPO + A12TNF |
| 2818 | CAD ~ Intercept.Low + AdipA1 + NTproBNP + PlGF |
| 2819 | CAD ~ Intercept.Low + AdipA1 + NTproBNP + A8MPO |
| 2820 | CAD ~ Intercept.Low + AdipA1 + NTproBNP + A12TNF |
| 2821 | CAD ~ Intercept.Low + AdipA1 + PlGF + A8MPO |
| 2822 | CAD ~ Intercept.Low + AdipA1 + PlGF + A12TNF |
| 2823 | CAD ~ Intercept.Low + AdipA1 + A8MPO + A12TNF |
| 2824 | CAD ~ Intercept.Low + NTproBNP + PlGF + A8MPO |
| 2825 | CAD ~ Intercept.Low + NTproBNP + PlGF + A12TNF |
| 2826 | CAD ~ Intercept.Low + NTproBNP + A8MPO + A12TNF |
| 2827 | CAD ~ Intercept.Low + PlGF + A8MPO + A12TNF |
| 2828 | CAD ~ Intercept.Low + AdipA1 + NTproBNP + PlGF + A8MPO |
| 2829 | CAD ~ Intercept.Low + AdipA1 + NTproBNP + PlGF + A12TNF |
| 2830 | CAD ~ Intercept.Low + AdipA1 + NTproBNP + A8MPO + A12TNF |
| 2831 | CAD ~ Intercept.Low + AdipA1 + PlGF + A8MPO + A12TNF |
| 2832 | CAD ~ Intercept.Low + NTproBNP + PlGF + A8MPO + A12TNF |
| 2833 | CAD ~ Intercept.Low + NTproBNP |
| 2834 | CAD ~ Intercept.Low + AdipA1 |
| 2835 | CAD ~ Intercept.Low + PlGF |
| 2836 | CAD ~ Intercept.Low + A8MPO |
| 2837 | CAD ~ Intercept.Low + A12TNF |
| 2838 | CAD ~ Intercept.Low + NTproBNP + PlGF |
| 2839 | CAD ~ Intercept.Low + AdipA1 + NTproBNP |
| 2840 | CAD ~ Intercept.Low + AdipA1 + PlGF |
| 2841 | CAD ~ Intercept.Low + AdipA1 + A8MPO |
| 2842 | CAD ~ Intercept.Low + AdipA1 + A12TNF |
| 2843 | CAD ~ Intercept.Low + NTproBNP + A8MPO |
| 2844 | CAD ~ Intercept.Low + NTproBNP + A12TNF |
| 2845 | CAD ~ Intercept.Low + PlGF + A8MPO |
| 2846 | CAD ~ Intercept.Low + PlGF + A12TNF |
| 2847 | CAD ~ Intercept.Low + A8MPO + A12TNF |
| 2848 | CAD ~ Intercept.Low + NTproBNP + PlGF |
| 2849 | CAD ~ Intercept.Low + AdipA1 + NTproBNP |
| 2850 | CAD ~ Intercept.Low + AdipA1 + PlGF |
| 2851 | CAD ~ Intercept.Low + AdipA1 + A8MPO |
| 2852 | CAD ~ Intercept.Low + AdipA1 + A12TNF |
| 2853 | CAD ~ Intercept.Low + NTproBNP + A8MPO |
| 2854 | CAD ~ Intercept.Low + NTproBNP + A12TNF |
| 2855 | CAD ~ Intercept.Low + PlGF + A8MPO |
| 2856 | CAD ~ Intercept.Low + PlGF + A12TNF |
| 2857 | CAD ~ Intercept.Low + A8MPO + A12TNF |
| 2858 | CAD ~ Intercept.Low + AdipA1 + NTproBNP + PlGF |
| 2859 | CAD ~ Intercept.Low + AdipA1 + NTproBNP + A8MPO |
| 2860 | CAD ~ Intercept.Low + AdipA1 + NTproBNP + A12TNF |
| 2861 | CAD ~ Intercept.Low + AdipA1 + PlGF + A8MPO |
| 2862 | CAD ~ Intercept.Low + AdipA1 + PlGF + A12TNF |
| 2863 | CAD ~ Intercept.Low + AdipA1 + A8MPO + A12TNF |
| 2864 | CAD ~ Intercept.Low + NTproBNP + PlGF + A8MPO |
| 2865 | CAD ~ Intercept.Low + NTproBNP + PlGF + A12TNF |
| 2866 | CAD ~ Intercept.Low + NTproBNP + A8MPO + A12TNF |
| 2867 | CAD ~ Intercept.Low + PlGF + A8MPO + A12TNF |
| 2868 | CAD ~ Intercept.Low + NTproBNP + PlGF |
| 2869 | CAD ~ Intercept.Low + AdipA1 + NTproBNP |
| 2870 | CAD ~ Intercept.Low + AdipA1 + PlGF |
| 2871 | CAD ~ Intercept.Low + AdipA1 + A8MPO |
| 2872 | CAD ~ Intercept.Low + AdipA1 + A12TNF |
| 2873 | CAD ~ Intercept.Low + NTproBNP + A8MPO |
| 2874 | CAD ~ Intercept.Low + NTproBNP + A12TNF |
| 2875 | CAD ~ Intercept.Low + PlGF + A8MPO |
| 2876 | CAD ~ Intercept.Low + PlGF + A12TNF |
| 2877 | CAD ~ Intercept.Low + A8MPO + A12TNF |
| 2878 | CAD ~ Intercept.Low + AdipA1 + NTproBNP + PlGF |
| 2879 | CAD ~ Intercept.Low + AdipA1 + NTproBNP + A8MPO |
| 2880 | CAD ~ Intercept.Low + AdipA1 + NTproBNP + A12TNF |
| 2881 | CAD ~ Intercept.Low + AdipA1 + PlGF + A8MPO |
| 2882 | CAD ~ Intercept.Low + AdipA1 + PlGF + A12TNF |
| 2883 | CAD ~ Intercept.Low + AdipA1 + A8MPO + A12TNF |
| 2884 | CAD ~ Intercept.Low + NTproBNP + PlGF + A8MPO |
| 2885 | CAD ~ Intercept.Low + NTproBNP + PlGF + A12TNF |
| 2886 | CAD ~ Intercept.Low + NTproBNP + A8MPO + A12TNF |
| 2887 | CAD ~ Intercept.Low + PlGF + A8MPO + A12TNF |
| 2888 | CAD ~ Intercept.Low + AdipA1 + NTproBNP + PlGF |
| 2889 | CAD ~ Intercept.Low + AdipA1 + NTproBNP + A8MPO |
| 2890 | CAD ~ Intercept.Low + AdipA1 + NTproBNP + A12TNF |
| 2891 | CAD ~ Intercept.Low + AdipA1 + PlGF + A8MPO |
| 2892 | CAD ~ Intercept.Low + AdipA1 + PlGF + A12TNF |
| 2893 | CAD ~ Intercept.Low + AdipA1 + A8MPO + A12TNF |
| 2894 | CAD ~ Intercept.Low + NTproBNP + PlGF + A8MPO |
| 2895 | CAD ~ Intercept.Low + NTproBNP + PlGF + A12TNF |
| 2896 | CAD ~ Intercept.Low + NTproBNP + A8MPO + A12TNF |
| 2897 | CAD ~ Intercept.Low + PlGF + A8MPO + A12TNF |
| 2898 | CAD ~ Intercept.Low + AdipA1 + NTproBNP + PlGF + A8MPO |
| 2899 | CAD ~ Intercept.Low + AdipA1 + NTproBNP + PlGF + A12TNF |
| 2900 | CAD ~ Intercept.Low + AdipA1 + NTproBNP + A8MPO + A12TNF |
| 2901 | CAD ~ Intercept.Low + AdipA1 + PlGF + A8MPO + A12TNF |
| 2902 | CAD ~ Intercept.Low + NTproBNP + PlGF + A8MPO + A12TNF |
| 2903 | CAD ~ Intercept.Low + NTproBNP |
| 2904 | CAD ~ Intercept.Low + AdipA1 |
| 2905 | CAD ~ Intercept.Low + PlGF |
| 2906 | CAD ~ Intercept.Low + A8MPO |
| 2907 | CAD ~ Intercept.Low + A12TNF |
| 2908 | CAD ~ Intercept.Low + NTproBNP |
| 2909 | CAD ~ Intercept.Low + AdipA1 |
| 2910 | CAD ~ Intercept.Low + PlGF |
| 2911 | CAD ~ Intercept.Low + A8MPO |
| 2912 | CAD ~ Intercept.Low + A12TNF |
| 2913 | CAD ~ Intercept.Low + NTproBNP |
| 2914 | CAD ~ Intercept.Low + AdipA1 |
| 2915 | CAD ~ Intercept.Low + PlGF |
| 2916 | CAD ~ Intercept.Low + A8MPO |
| 2917 | CAD ~ Intercept.Low + A12TNF |
| 2918 | CAD ~ Intercept.Low + NTproBNP |
| 2919 | CAD ~ Intercept.Low + AdipA1 |
| 2920 | CAD ~ Intercept.Low + PlGF |
| 2921 | CAD ~ Intercept.Low + A8MPO |
| 2922 | CAD ~ Intercept.Low + A12TNF |
| 2923 | CAD ~ Intercept.Low + NTproBNP + PlGF |
| 2924 | CAD ~ Intercept.Low + AdipA1 + NTproBNP |
| 2925 | CAD ~ Intercept.Low + AdipA1 + PlGF |
| 2926 | CAD ~ Intercept.Low + AdipA1 + A8MPO |
| 2927 | CAD ~ Intercept.Low + AdipA1 + A12TNF |
| 2928 | CAD ~ Intercept.Low + NTproBNP + A8MPO |
| 2929 | CAD ~ Intercept.Low + NTproBNP + A12TNF |
| 2930 | CAD ~ Intercept.Low + PlGF + A8MPO |
| 2931 | CAD ~ Intercept.Low + PlGF + A12TNF |
| 2932 | CAD ~ Intercept.Low + A8MPO + A12TNF |
| 2933 | CAD ~ Intercept.Low + NTproBNP |
| 2934 | CAD ~ Intercept.Low + AdipA1 |
| 2935 | CAD ~ Intercept.Low + PlGF |
| 2936 | CAD ~ Intercept.Low + A8MPO |
| 2937 | CAD ~ Intercept.Low + A12TNF |
| 2938 | CAD ~ Intercept.Low + NTproBNP |
| 2939 | CAD ~ Intercept.Low + AdipA1 |
| 2940 | CAD ~ Intercept.Low + PlGF |
| 2941 | CAD ~ Intercept.Low + A8MPO |
| 2942 | CAD ~ Intercept.Low + A12TNF |
| 2943 | CAD ~ Intercept.Low + NTproBNP |
| 2944 | CAD ~ Intercept.Low + AdipA1 |
| 2945 | CAD ~ Intercept.Low + PlGF |
| 2946 | CAD ~ Intercept.Low + A8MPO |
| 2947 | CAD ~ Intercept.Low + A12TNF |
| 2948 | CAD ~ Intercept.Low + NTproBNP + PlGF |
| 2949 | CAD ~ Intercept.Low + AdipA1 + NTproBNP |
| 2950 | CAD ~ Intercept.Low + AdipA1 + PlGF |
| 2951 | CAD ~ Intercept.Low + AdipA1 + A8MPO |
| 2952 | CAD ~ Intercept.Low + AdipA1 + A12TNF |
| 2953 | CAD ~ Intercept.Low + NTproBNP + A8MPO |
| 2954 | CAD ~ Intercept.Low + NTproBNP + A12TNF |
| 2955 | CAD ~ Intercept.Low + PlGF + A8MPO |
| 2956 | CAD ~ Intercept.Low + PlGF + A12TNF |
| 2957 | CAD ~ Intercept.Low + A8MPO + A12TNF |
| 2958 | CAD ~ Intercept.Low + NTproBNP |
| 2959 | CAD ~ Intercept.Low + AdipA1 |
| 2960 | CAD ~ Intercept.Low + PlGF |
| 2961 | CAD ~ Intercept.Low + A8MPO |
| 2962 | CAD ~ Intercept.Low + A12TNF |
| 2963 | CAD ~ Intercept.Low + NTproBNP |
| 2964 | CAD ~ Intercept.Low + AdipA1 |
| 2965 | CAD ~ Intercept.Low + PlGF |
| 2966 | CAD ~ Intercept.Low + A8MPO |
| 2967 | CAD ~ Intercept.Low + A12TNF |
| 2968 | CAD ~ Intercept.Low + NTproBNP + PlGF |
| 2969 | CAD ~ Intercept.Low + AdipA1 + NTproBNP |
| 2970 | CAD ~ Intercept.Low + AdipA1 + PlGF |
| 2971 | CAD ~ Intercept.Low + AdipA1 + A8MPO |
| 2972 | CAD ~ Intercept.Low + AdipA1 + A12TNF |
| 2973 | CAD ~ Intercept.Low + NTproBNP + A8MPO |
| 2974 | CAD ~ Intercept.Low + NTproBNP + A12TNF |
| 2975 | CAD ~ Intercept.Low + PlGF + A8MPO |
| 2976 | CAD ~ Intercept.Low + PlGF + A12TNF |
| 2977 | CAD ~ Intercept.Low + A8MPO + A12TNF |
| 2978 | CAD ~ Intercept.Low + NTproBNP |
| 2979 | CAD ~ Intercept.Low + AdipA1 |
| 2980 | CAD ~ Intercept.Low + PlGF |
| 2981 | CAD ~ Intercept.Low + A8MPO |
| 2982 | CAD ~ Intercept.Low + A12TNF |
| 2983 | CAD ~ Intercept.Low + NTproBNP + PlGF |
| 2984 | CAD ~ Intercept.Low + AdipA1 + NTproBNP |
| 2985 | CAD ~ Intercept.Low + AdipA1 + PlGF |
| 2986 | CAD ~ Intercept.Low + AdipA1 + A8MPO |
| 2987 | CAD ~ Intercept.Low + AdipA1 + A12TNF |
| 2988 | CAD ~ Intercept.Low + NTproBNP + A8MPO |
| 2989 | CAD ~ Intercept.Low + NTproBNP + A12TNF |
| 2990 | CAD ~ Intercept.Low + PlGF + A8MPO |
| 2991 | CAD ~ Intercept.Low + PlGF + A12TNF |
| 2992 | CAD ~ Intercept.Low + A8MPO + A12TNF |
| 2993 | CAD ~ Intercept.Low + NTproBNP + PlGF |
| 2994 | CAD ~ Intercept.Low + AdipA1 + NTproBNP |
| 2995 | CAD ~ Intercept.Low + AdipA1 + PlGF |
| 2996 | CAD ~ Intercept.Low + AdipA1 + A8MPO |
| 2997 | CAD ~ Intercept.Low + AdipA1 + A12TNF |
| 2998 | CAD ~ Intercept.Low + NTproBNP + A8MPO |
| 2999 | CAD ~ Intercept.Low + NTproBNP + A12TNF |
| 3000 | CAD ~ Intercept.Low + PlGF + A8MPO |
| 3001 | CAD ~ Intercept.Low + PlGF + A12TNF |
| 3002 | CAD ~ Intercept.Low + A8MPO + A12TNF |
| 3003 | CAD ~ Intercept.Low + AdipA1 + NTproBNP + PlGF |
| 3004 | CAD ~ Intercept.Low + AdipA1 + NTproBNP + A8MPO |
| 3005 | CAD ~ Intercept.Low + AdipA1 + NTproBNP + A12TNF |
| 3006 | CAD ~ Intercept.Low + AdipA1 + PlGF + A8MPO |
| 3007 | CAD ~ Intercept.Low + AdipA1 + PlGF + A12TNF |
| 3008 | CAD ~ Intercept.Low + AdipA1 + A8MPO + A12TNF |
| 3009 | CAD ~ Intercept.Low + NTproBNP + PlGF + A8MPO |
| 3010 | CAD ~ Intercept.Low + NTproBNP + PlGF + A12TNF |
| 3011 | CAD ~ Intercept.Low + NTproBNP + A8MPO + A12TNF |
| 3012 | CAD ~ Intercept.Low + PlGF + A8MPO + A12TNF |
| 3013 | CAD ~ Intercept.Low + NTproBNP |
| 3014 | CAD ~ Intercept.Low + AdipA1 |
| 3015 | CAD ~ Intercept.Low + PlGF |
| 3016 | CAD ~ Intercept.Low + A8MPO |
| 3017 | CAD ~ Intercept.Low + A12TNF |
| 3018 | CAD ~ Intercept.Low + NTproBNP |
| 3019 | CAD ~ Intercept.Low + AdipA1 |
| 3020 | CAD ~ Intercept.Low + PlGF |
| 3021 | CAD ~ Intercept.Low + A8MPO |
| 3022 | CAD ~ Intercept.Low + A12TNF |
| 3023 | CAD ~ Intercept.Low + NTproBNP |
| 3024 | CAD ~ Intercept.Low + AdipA1 |
| 3025 | CAD ~ Intercept.Low + PlGF |
| 3026 | CAD ~ Intercept.Low + A8MPO |
| 3027 | CAD ~ Intercept.Low + A12TNF |
| 3028 | CAD ~ Intercept.Low + NTproBNP + PlGF |
| 3029 | CAD ~ Intercept.Low + AdipA1 + NTproBNP |
| 3030 | CAD ~ Intercept.Low + AdipA1 + PlGF |
| 3031 | CAD ~ Intercept.Low + AdipA1 + A8MPO |
| 3032 | CAD ~ Intercept.Low + AdipA1 + A12TNF |
| 3033 | CAD ~ Intercept.Low + NTproBNP + A8MPO |
| 3034 | CAD ~ Intercept.Low + NTproBNP + A12TNF |
| 3035 | CAD ~ Intercept.Low + PlGF + A8MPO |
| 3036 | CAD ~ Intercept.Low + PlGF + A12TNF |
| 3037 | CAD ~ Intercept.Low + A8MPO + A12TNF |
| 3038 | CAD ~ Intercept.Low + NTproBNP |
| 3039 | CAD ~ Intercept.Low + AdipA1 |
| 3040 | CAD ~ Intercept.Low + PlGF |
| 3041 | CAD ~ Intercept.Low + A8MPO |
| 3042 | CAD ~ Intercept.Low + A12TNF |
| 3043 | CAD ~ Intercept.Low + NTproBNP |
| 3044 | CAD ~ Intercept.Low + AdipA1 |
| 3045 | CAD ~ Intercept.Low + PlGF |
| 3046 | CAD ~ Intercept.Low + A8MPO |
| 3047 | CAD ~ Intercept.Low + A12TNF |
| 3048 | CAD ~ Intercept.Low + NTproBNP + PlGF |
| 3049 | CAD ~ Intercept.Low + AdipA1 + NTproBNP |
| 3050 | CAD ~ Intercept.Low + AdipA1 + PlGF |
| 3051 | CAD ~ Intercept.Low + AdipA1 + A8MPO |
| 3052 | CAD ~ Intercept.Low + AdipA1 + A12TNF |
| 3053 | CAD ~ Intercept.Low + NTproBNP + A8MPO |
| 3054 | CAD ~ Intercept.Low + NTproBNP + A12TNF |
| 3055 | CAD ~ Intercept.Low + PlGF + A8MPO |
| 3056 | CAD ~ Intercept.Low + PlGF + A12TNF |
| 3057 | CAD ~ Intercept.Low + A8MPO + A12TNF |
| 3058 | CAD ~ Intercept.Low + NTproBNP |
| 3059 | CAD ~ Intercept.Low + AdipA1 |
| 3060 | CAD ~ Intercept.Low + PlGF |
| 3061 | CAD ~ Intercept.Low + A8MPO |
| 3062 | CAD ~ Intercept.Low + A12TNF |
| 3063 | CAD ~ Intercept.Low + NTproBNP + PlGF |
| 3064 | CAD ~ Intercept.Low + AdipA1 + NTproBNP |
| 3065 | CAD ~ Intercept.Low + AdipA1 + PlGF |
| 3066 | CAD ~ Intercept.Low + AdipA1 + A8MPO |
| 3067 | CAD ~ Intercept.Low + AdipA1 + A12TNF |
| 3068 | CAD ~ Intercept.Low + NTproBNP + A8MPO |
| 3069 | CAD ~ Intercept.Low + NTproBNP + A12TNF |
| 3070 | CAD ~ Intercept.Low + PlGF + A8MPO |
| 3071 | CAD ~ Intercept.Low + PlGF + A12TNF |
| 3072 | CAD ~ Intercept.Low + A8MPO + A12TNF |
| 3073 | CAD ~ Intercept.Low + NTproBNP + PlGF |
| 3074 | CAD ~ Intercept.Low + AdipA1 + NTproBNP |
| 3075 | CAD ~ Intercept.Low + AdipA1 + PlGF |
| 3076 | CAD ~ Intercept.Low + AdipA1 + A8MPO |
| 3077 | CAD ~ Intercept.Low + AdipA1 + A12TNF |
| 3078 | CAD ~ Intercept.Low + NTproBNP + A8MPO |
| 3079 | CAD ~ Intercept.Low + NTproBNP + A12TNF |
| 3080 | CAD ~ Intercept.Low + PlGF + A8MPO |
| 3081 | CAD ~ Intercept.Low + PlGF + A12TNF |
| 3082 | CAD ~ Intercept.Low + A8MPO + A12TNF |
| 3083 | CAD ~ Intercept.Low + AdipA1 + NTproBNP + PlGF |
| 3084 | CAD ~ Intercept.Low + AdipA1 + NTproBNP + A8MPO |
| 3085 | CAD ~ Intercept.Low + AdipA1 + NTproBNP + A12TNF |
| 3086 | CAD ~ Intercept.Low + AdipA1 + PlGF + A8MPO |
| 3087 | CAD ~ Intercept.Low + AdipA1 + PlGF + A12TNF |
| 3088 | CAD ~ Intercept.Low + AdipA1 + A8MPO + A12TNF |
| 3089 | CAD ~ Intercept.Low + NTproBNP + PlGF + A8MPO |
| 3090 | CAD ~ Intercept.Low + NTproBNP + PlGF + A12TNF |
| 3091 | CAD ~ Intercept.Low + NTproBNP + A8MPO + A12TNF |
| 3092 | CAD ~ Intercept.Low + PlGF + A8MPO + A12TNF |
| 3093 | CAD ~ Intercept.Low + NTproBNP |
| 3094 | CAD ~ Intercept.Low + AdipA1 |
| 3095 | CAD ~ Intercept.Low + PlGF |
| 3096 | CAD ~ Intercept.Low + A8MPO |
| 3097 | CAD ~ Intercept.Low + A12TNF |
| 3098 | CAD ~ Intercept.Low + NTproBNP |
| 3099 | CAD ~ Intercept.Low + AdipA1 |
| 3100 | CAD ~ Intercept.Low + PlGF |
| 3101 | CAD ~ Intercept.Low + A8MPO |
| 3102 | CAD ~ Intercept.Low + A12TNF |
| 3103 | CAD ~ Intercept.Low + NTproBNP + PlGF |
| 3104 | CAD ~ Intercept.Low + AdipA1 + NTproBNP |
| 3105 | CAD ~ Intercept.Low + AdipA1 + PlGF |
| 3106 | CAD ~ Intercept.Low + AdipA1 + A8MPO |
| 3107 | CAD ~ Intercept.Low + AdipA1 + A12TNF |
| 3108 | CAD ~ Intercept.Low + NTproBNP + A8MPO |
| 3109 | CAD ~ Intercept.Low + NTproBNP + A12TNF |
| 3110 | CAD ~ Intercept.Low + PlGF + A8MPO |
| 3111 | CAD ~ Intercept.Low + PlGF + A12TNF |
| 3112 | CAD ~ Intercept.Low + A8MPO + A12TNF |
| 3113 | CAD ~ Intercept.Low + NTproBNP |
| 3114 | CAD ~ Intercept.Low + AdipA1 |
| 3115 | CAD ~ Intercept.Low + PlGF |
| 3116 | CAD ~ Intercept.Low + A8MPO |
| 3117 | CAD ~ Intercept.Low + A12TNF |
| 3118 | CAD ~ Intercept.Low + NTproBNP + PlGF |
| 3119 | CAD ~ Intercept.Low + AdipA1 + NTproBNP |
| 3120 | CAD ~ Intercept.Low + AdipA1 + PlGF |
| 3121 | CAD ~ Intercept.Low + AdipA1 + A8MPO |
| 3122 | CAD ~ Intercept.Low + AdipA1 + A12TNF |
| 3123 | CAD ~ Intercept.Low + NTproBNP + A8MPO |
| 3124 | CAD ~ Intercept.Low + NTproBNP + A12TNF |
| 3125 | CAD ~ Intercept.Low + PlGF + A8MPO |
| 3126 | CAD ~ Intercept.Low + PlGF + A12TNF |
| 3127 | CAD ~ Intercept.Low + A8MPO + A12TNF |
| 3128 | CAD ~ Intercept.Low + NTproBNP + PlGF |
| 3129 | CAD ~ Intercept.Low + AdipA1 + NTproBNP |
| 3130 | CAD ~ Intercept.Low + AdipA1 + PlGF |
| 3131 | CAD ~ Intercept.Low + AdipA1 + A8MPO |
| 3132 | CAD ~ Intercept.Low + AdipA1 + A12TNF |
| 3133 | CAD ~ Intercept.Low + NTproBNP + A8MPO |
| 3134 | CAD ~ Intercept.Low + NTproBNP + A12TNF |
| 3135 | CAD ~ Intercept.Low + PlGF + A8MPO |
| 3136 | CAD ~ Intercept.Low + PlGF + A12TNF |
| 3137 | CAD ~ Intercept.Low + A8MPO + A12TNF |
| 3138 | CAD ~ Intercept.Low + AdipA1 + NTproBNP + PlGF |
| 3139 | CAD ~ Intercept.Low + AdipA1 + NTproBNP + A8MPO |
| 3140 | CAD ~ Intercept.Low + AdipA1 + NTproBNP + A12TNF |
| 3141 | CAD ~ Intercept.Low + AdipA1 + PlGF + A8MPO |
| 3142 | CAD ~ Intercept.Low + AdipA1 + PlGF + A12TNF |
| 3143 | CAD ~ Intercept.Low + AdipA1 + A8MPO + A12TNF |
| 3144 | CAD ~ Intercept.Low + NTproBNP + PlGF + A8MPO |
| 3145 | CAD ~ Intercept.Low + NTproBNP + PlGF + A12TNF |
| 3146 | CAD ~ Intercept.Low + NTproBNP + A8MPO + A12TNF |
| 3147 | CAD ~ Intercept.Low + PlGF + A8MPO + A12TNF |
| 3148 | CAD ~ Intercept.Low + NTproBNP |
| 3149 | CAD ~ Intercept.Low + AdipA1 |
| 3150 | CAD ~ Intercept.Low + PlGF |
| 3151 | CAD ~ Intercept.Low + A8MPO |
| 3152 | CAD ~ Intercept.Low + A12TNF |
| 3153 | CAD ~ Intercept.Low + NTproBNP + PlGF |
| 3154 | CAD ~ Intercept.Low + AdipA1 + NTproBNP |
| 3155 | CAD ~ Intercept.Low + AdipA1 + PlGF |
| 3156 | CAD ~ Intercept.Low + AdipA1 + A8MPO |
| 3157 | CAD ~ Intercept.Low + AdipA1 + A12TNF |
| 3158 | CAD ~ Intercept.Low + NTproBNP + A8MPO |
| 3159 | CAD ~ Intercept.Low + NTproBNP + A12TNF |
| 3160 | CAD ~ Intercept.Low + PlGF + A8MPO |
| 3161 | CAD ~ Intercept.Low + PlGF + A12TNF |
| 3162 | CAD ~ Intercept.Low + A8MPO + A12TNF |
| 3163 | CAD ~ Intercept.Low + NTproBNP + PlGF |
| 3164 | CAD ~ Intercept.Low + AdipA1 + NTproBNP |
| 3165 | CAD ~ Intercept.Low + AdipA1 + PlGF |
| 3166 | CAD ~ Intercept.Low + AdipA1 + A8MPO |
| 3167 | CAD ~ Intercept.Low + AdipA1 + A12TNF |
| 3168 | CAD ~ Intercept.Low + NTproBNP + A8MPO |
| 3169 | CAD ~ Intercept.Low + NTproBNP + A12TNF |
| 3170 | CAD ~ Intercept.Low + PlGF + A8MPO |
| 3171 | CAD ~ Intercept.Low + PlGF + A12TNF |
| 3172 | CAD ~ Intercept.Low + A8MPO + A12TNF |
| 3173 | CAD ~ Intercept.Low + AdipA1 + NTproBNP + PlGF |
| 3174 | CAD ~ Intercept.Low + AdipA1 + NTproBNP + A8MPO |
| 3175 | CAD ~ Intercept.Low + AdipA1 + NTproBNP + A12TNF |
| 3176 | CAD ~ Intercept.Low + AdipA1 + PlGF + A8MPO |
| 3177 | CAD ~ Intercept.Low + AdipA1 + PlGF + A12TNF |
| 3178 | CAD ~ Intercept.Low + AdipA1 + A8MPO + A12TNF |
| 3179 | CAD ~ Intercept.Low + NTproBNP + PlGF + A8MPO |
| 3180 | CAD ~ Intercept.Low + NTproBNP + PlGF + A12TNF |
| 3181 | CAD ~ Intercept.Low + NTproBNP + A8MPO + A12TNF |
| 3182 | CAD ~ Intercept.Low + PlGF + A8MPO + A12TNF |
| 3183 | CAD ~ Intercept.Low + NTproBNP + PlGF |
| 3184 | CAD ~ Intercept.Low + AdipA1 + NTproBNP |
| 3185 | CAD ~ Intercept.Low + AdipA1 + PlGF |
| 3186 | CAD ~ Intercept.Low + AdipA1 + A8MPO |
| 3187 | CAD ~ Intercept.Low + AdipA1 + A12TNF |
| 3188 | CAD ~ Intercept.Low + NTproBNP + A8MPO |
| 3189 | CAD ~ Intercept.Low + NTproBNP + A12TNF |
| 3190 | CAD ~ Intercept.Low + PlGF + A8MPO |
| 3191 | CAD ~ Intercept.Low + PlGF + A12TNF |
| 3192 | CAD ~ Intercept.Low + A8MPO + A12TNF |
| 3193 | CAD ~ Intercept.Low + AdipA1 + NTproBNP + PlGF |
| 3194 | CAD ~ Intercept.Low + AdipA1 + NTproBNP + A8MPO |
| 3195 | CAD ~ Intercept.Low + AdipA1 + NTproBNP + A12TNF |
| 3196 | CAD ~ Intercept.Low + AdipA1 + PlGF + A8MPO |
| 3197 | CAD ~ Intercept.Low + AdipA1 + PlGF + A12TNF |
| 3198 | CAD ~ Intercept.Low + AdipA1 + A8MPO + A12TNF |
| 3199 | CAD ~ Intercept.Low + NTproBNP + PlGF + A8MPO |
| 3200 | CAD ~ Intercept.Low + NTproBNP + PlGF + A12TNF |
| 3201 | CAD ~ Intercept.Low + NTproBNP + A8MPO + A12TNF |
| 3202 | CAD ~ Intercept.Low + PlGF + A8MPO + A12TNF |
| 3203 | CAD ~ Intercept.Low + AdipA1 + NTproBNP + PlGF |
| 3204 | CAD ~ Intercept.Low + AdipA1 + NTproBNP + A8MPO |
| 3205 | CAD ~ Intercept.Low + AdipA1 + NTproBNP + A12TNF |
| 3206 | CAD ~ Intercept.Low + AdipA1 + PlGF + A8MPO |
| 3207 | CAD ~ Intercept.Low + AdipA1 + PlGF + A12TNF |
| 3208 | CAD ~ Intercept.Low + AdipA1 + A8MPO + A12TNF |
| 3209 | CAD ~ Intercept.Low + NTproBNP + PlGF + A8MPO |
| 3210 | CAD ~ Intercept.Low + NTproBNP + PlGF + A12TNF |
| 3211 | CAD ~ Intercept.Low + NTproBNP + A8MPO + A12TNF |
| 3212 | CAD ~ Intercept.Low + PlGF + A8MPO + A12TNF |
| 3213 | CAD ~ Intercept.Low + AdipA1 + NTproBNP + PlGF + A8MPO |
| 3214 | CAD ~ Intercept.Low + AdipA1 + NTproBNP + PlGF + A12TNF |
| 3215 | CAD ~ Intercept.Low + AdipA1 + NTproBNP + A8MPO + A12TNF |
| 3216 | CAD ~ Intercept.Low + AdipA1 + PlGF + A8MPO + A12TNF |
| 3217 | CAD ~ Intercept.Low + NTproBNP + PlGF + A8MPO + A12TNF |
| 3218 | CAD ~ Intercept.Low + NTproBNP + PlGF |
| 3219 | CAD ~ Intercept.Low + AdipA1 + NTproBNP |
| 3220 | CAD ~ Intercept.Low + AdipA1 + PlGF |
| 3221 | CAD ~ Intercept.Low + AdipA1 + A8MPO |
| 3222 | CAD ~ Intercept.Low + AdipA1 + A12TNF |
| 3223 | CAD ~ Intercept.Low + NTproBNP + A8MPO |
| 3224 | CAD ~ Intercept.Low + NTproBNP + A12TNF |
| 3225 | CAD ~ Intercept.Low + PlGF + A8MPO |
| 3226 | CAD ~ Intercept.Low + PlGF + A12TNF |
| 3227 | CAD ~ Intercept.Low + A8MPO + A12TNF |
| 3228 | CAD ~ Intercept.Low + AdipA1 + NTproBNP + PlGF |
| 3229 | CAD ~ Intercept.Low + AdipA1 + NTproBNP + A8MPO |
| 3230 | CAD ~ Intercept.Low + AdipA1 + NTproBNP + A12TNF |
| 3231 | CAD ~ Intercept.Low + AdipA1 + PlGF + A8MPO |
| 3232 | CAD ~ Intercept.Low + AdipA1 + PlGF + A12TNF |
| 3233 | CAD ~ Intercept.Low + AdipA1 + A8MPO + A12TNF |
| 3234 | CAD ~ Intercept.Low + NTproBNP + PlGF + A8MPO |
| 3235 | CAD ~ Intercept.Low + NTproBNP + PlGF + A12TNF |
| 3236 | CAD ~ Intercept.Low + NTproBNP + A8MPO + A12TNF |
| 3237 | CAD ~ Intercept.Low + PlGF + A8MPO + A12TNF |
| 3238 | CAD ~ Intercept.Low + AdipA1 + NTproBNP + PlGF |
| 3239 | CAD ~ Intercept.Low + AdipA1 + NTproBNP + A8MPO |
| 3240 | CAD ~ Intercept.Low + AdipA1 + NTproBNP + A12TNF |
| 3241 | CAD ~ Intercept.Low + AdipA1 + PlGF + A8MPO |
| 3242 | CAD ~ Intercept.Low + AdipA1 + PlGF + A12TNF |
| 3243 | CAD ~ Intercept.Low + AdipA1 + A8MPO + A12TNF |
| 3244 | CAD ~ Intercept.Low + NTproBNP + PlGF + A8MPO |
| 3245 | CAD ~ Intercept.Low + NTproBNP + PlGF + A12TNF |
| 3246 | CAD ~ Intercept.Low + NTproBNP + A8MPO + A12TNF |
| 3247 | CAD ~ Intercept.Low + PlGF + A8MPO + A12TNF |
| 3248 | CAD ~ Intercept.Low + AdipA1 + NTproBNP + PlGF + A8MPO |
| 3249 | CAD ~ Intercept.Low + AdipA1 + NTproBNP + PlGF + A12TNF |
| 3250 | CAD ~ Intercept.Low + AdipA1 + NTproBNP + A8MPO + A12TNF |
| 3251 | CAD ~ Intercept.Low + AdipA1 + PlGF + A8MPO + A12TNF |
| 3252 | CAD ~ Intercept.Low + NTproBNP + PlGF + A8MPO + A12TNF |
| 3253 | CAD ~ Intercept.Low + AdipA1 + NTproBNP + PlGF |
| 3254 | CAD ~ Intercept.Low + AdipA1 + NTproBNP + A8MPO |
| 3255 | CAD ~ Intercept.Low + AdipA1 + NTproBNP + A12TNF |
| 3256 | CAD ~ Intercept.Low + AdipA1 + PlGF + A8MPO |
| 3257 | CAD ~ Intercept.Low + AdipA1 + PlGF + A12TNF |
| 3258 | CAD ~ Intercept.Low + AdipA1 + A8MPO + A12TNF |
| 3259 | CAD ~ Intercept.Low + NTproBNP + PlGF + A8MPO |
| 3260 | CAD ~ Intercept.Low + NTproBNP + PlGF + A12TNF |
| 3261 | CAD ~ Intercept.Low + NTproBNP + A8MPO + A12TNF |
| 3262 | CAD ~ Intercept.Low + PlGF + A8MPO + A12TNF |
| 3263 | CAD ~ Intercept.Low + AdipA1 + NTproBNP + PlGF + A8MPO |
| 3264 | CAD ~ Intercept.Low + AdipA1 + NTproBNP + PlGF + A12TNF |
| 3265 | CAD ~ Intercept.Low + AdipA1 + NTproBNP + A8MPO + A12TNF |
| 3266 | CAD ~ Intercept.Low + AdipA1 + PlGF + A8MPO + A12TNF |
| 3267 | CAD ~ Intercept.Low + NTproBNP + PlGF + A8MPO + A12TNF |
| 3268 | CAD ~ Intercept.Low + AdipA1 + NTproBNP + PlGF + A8MPO |
| 3269 | CAD ~ Intercept.Low + AdipA1 + NTproBNP + PlGF + A12TNF |
| 3270 | CAD ~ Intercept.Low + AdipA1 + NTproBNP + A8MPO + A12TNF |
| 3271 | CAD ~ Intercept.Low + AdipA1 + PlGF + A8MPO + A12TNF |
| 3272 | CAD ~ Intercept.Low + NTproBNP + PlGF + A8MPO + A12TNF |
| 3273 | NA |
| 3274 | CAD ~ Intercept.Low + AdipA1 + NTproBNP + PlGF + A8MPO + A12TNF |
| 3275 | CAD ~ Intercept.Low + NTproBNP |
| 3276 | CAD ~ Intercept.Low + AdipA1 |
| 3277 | CAD ~ Intercept.Low + PlGF |
| 3278 | CAD ~ Intercept.Low + A8MPO |
| 3279 | CAD ~ Intercept.Low + A12TNF |
| 3280 | CAD ~ Intercept.Low + NTproBNP |
| 3281 | CAD ~ Intercept.Low + AdipA1 |
| 3282 | CAD ~ Intercept.Low + PlGF |
| 3283 | CAD ~ Intercept.Low + A8MPO |
| 3284 | CAD ~ Intercept.Low + A12TNF |
| 3285 | CAD ~ Intercept.Low + NTproBNP |
| 3286 | CAD ~ Intercept.Low + AdipA1 |
| 3287 | CAD ~ Intercept.Low + PlGF |
| 3288 | CAD ~ Intercept.Low + A8MPO |
| 3289 | CAD ~ Intercept.Low + A12TNF |
| 3290 | CAD ~ Intercept.Low + NTproBNP |
| 3291 | CAD ~ Intercept.Low + AdipA1 |
| 3292 | CAD ~ Intercept.Low + PlGF |
| 3293 | CAD ~ Intercept.Low + A8MPO |
| 3294 | CAD ~ Intercept.Low + A12TNF |
| 3295 | CAD ~ Intercept.Low + NTproBNP + PlGF |
| 3296 | CAD ~ Intercept.Low + AdipA1 + NTproBNP |
| 3297 | CAD ~ Intercept.Low + AdipA1 + PlGF |
| 3298 | CAD ~ Intercept.Low + AdipA1 + A8MPO |
| 3299 | CAD ~ Intercept.Low + AdipA1 + A12TNF |
| 3300 | CAD ~ Intercept.Low + NTproBNP + A8MPO |
| 3301 | CAD ~ Intercept.Low + NTproBNP + A12TNF |
| 3302 | CAD ~ Intercept.Low + PlGF + A8MPO |
| 3303 | CAD ~ Intercept.Low + PlGF + A12TNF |
| 3304 | CAD ~ Intercept.Low + A8MPO + A12TNF |
| 3305 | CAD ~ Intercept.Low + NTproBNP |
| 3306 | CAD ~ Intercept.Low + AdipA1 |
| 3307 | CAD ~ Intercept.Low + PlGF |
| 3308 | CAD ~ Intercept.Low + A8MPO |
| 3309 | CAD ~ Intercept.Low + A12TNF |
| 3310 | CAD ~ Intercept.Low + NTproBNP |
| 3311 | CAD ~ Intercept.Low + AdipA1 |
| 3312 | CAD ~ Intercept.Low + PlGF |
| 3313 | CAD ~ Intercept.Low + A8MPO |
| 3314 | CAD ~ Intercept.Low + A12TNF |
| 3315 | CAD ~ Intercept.Low + NTproBNP |
| 3316 | CAD ~ Intercept.Low + AdipA1 |
| 3317 | CAD ~ Intercept.Low + PlGF |
| 3318 | CAD ~ Intercept.Low + A8MPO |
| 3319 | CAD ~ Intercept.Low + A12TNF |
| 3320 | CAD ~ Intercept.Low + NTproBNP + PlGF |
| 3321 | CAD ~ Intercept.Low + AdipA1 + NTproBNP |
| 3322 | CAD ~ Intercept.Low + AdipA1 + PlGF |
| 3323 | CAD ~ Intercept.Low + AdipA1 + A8MPO |
| 3324 | CAD ~ Intercept.Low + AdipA1 + A12TNF |
| 3325 | CAD ~ Intercept.Low + NTproBNP + A8MPO |
| 3326 | CAD ~ Intercept.Low + NTproBNP + A12TNF |
| 3327 | CAD ~ Intercept.Low + PlGF + A8MPO |
| 3328 | CAD ~ Intercept.Low + PlGF + A12TNF |
| 3329 | CAD ~ Intercept.Low + A8MPO + A12TNF |
| 3330 | CAD ~ Intercept.Low + NTproBNP |
| 3331 | CAD ~ Intercept.Low + AdipA1 |
| 3332 | CAD ~ Intercept.Low + PlGF |
| 3333 | CAD ~ Intercept.Low + A8MPO |
| 3334 | CAD ~ Intercept.Low + A12TNF |
| 3335 | CAD ~ Intercept.Low + NTproBNP |
| 3336 | CAD ~ Intercept.Low + AdipA1 |
| 3337 | CAD ~ Intercept.Low + PlGF |
| 3338 | CAD ~ Intercept.Low + A8MPO |
| 3339 | CAD ~ Intercept.Low + A12TNF |
| 3340 | CAD ~ Intercept.Low + NTproBNP + PlGF |
| 3341 | CAD ~ Intercept.Low + AdipA1 + NTproBNP |
| 3342 | CAD ~ Intercept.Low + AdipA1 + PlGF |
| 3343 | CAD ~ Intercept.Low + AdipA1 + A8MPO |
| 3344 | CAD ~ Intercept.Low + AdipA1 + A12TNF |
| 3345 | CAD ~ Intercept.Low + NTproBNP + A8MPO |
| 3346 | CAD ~ Intercept.Low + NTproBNP + A12TNF |
| 3347 | CAD ~ Intercept.Low + PlGF + A8MPO |
| 3348 | CAD ~ Intercept.Low + PlGF + A12TNF |
| 3349 | CAD ~ Intercept.Low + A8MPO + A12TNF |
| 3350 | CAD ~ Intercept.Low + NTproBNP |
| 3351 | CAD ~ Intercept.Low + AdipA1 |
| 3352 | CAD ~ Intercept.Low + PlGF |
| 3353 | CAD ~ Intercept.Low + A8MPO |
| 3354 | CAD ~ Intercept.Low + A12TNF |
| 3355 | CAD ~ Intercept.Low + NTproBNP + PlGF |
| 3356 | CAD ~ Intercept.Low + AdipA1 + NTproBNP |
| 3357 | CAD ~ Intercept.Low + AdipA1 + PlGF |
| 3358 | CAD ~ Intercept.Low + AdipA1 + A8MPO |
| 3359 | CAD ~ Intercept.Low + AdipA1 + A12TNF |
| 3360 | CAD ~ Intercept.Low + NTproBNP + A8MPO |
| 3361 | CAD ~ Intercept.Low + NTproBNP + A12TNF |
| 3362 | CAD ~ Intercept.Low + PlGF + A8MPO |
| 3363 | CAD ~ Intercept.Low + PlGF + A12TNF |
| 3364 | CAD ~ Intercept.Low + A8MPO + A12TNF |
| 3365 | CAD ~ Intercept.Low + NTproBNP + PlGF |
| 3366 | CAD ~ Intercept.Low + AdipA1 + NTproBNP |
| 3367 | CAD ~ Intercept.Low + AdipA1 + PlGF |
| 3368 | CAD ~ Intercept.Low + AdipA1 + A8MPO |
| 3369 | CAD ~ Intercept.Low + AdipA1 + A12TNF |
| 3370 | CAD ~ Intercept.Low + NTproBNP + A8MPO |
| 3371 | CAD ~ Intercept.Low + NTproBNP + A12TNF |
| 3372 | CAD ~ Intercept.Low + PlGF + A8MPO |
| 3373 | CAD ~ Intercept.Low + PlGF + A12TNF |
| 3374 | CAD ~ Intercept.Low + A8MPO + A12TNF |
| 3375 | CAD ~ Intercept.Low + AdipA1 + NTproBNP + PlGF |
| 3376 | CAD ~ Intercept.Low + AdipA1 + NTproBNP + A8MPO |
| 3377 | CAD ~ Intercept.Low + AdipA1 + NTproBNP + A12TNF |
| 3378 | CAD ~ Intercept.Low + AdipA1 + PlGF + A8MPO |
| 3379 | CAD ~ Intercept.Low + AdipA1 + PlGF + A12TNF |
| 3380 | CAD ~ Intercept.Low + AdipA1 + A8MPO + A12TNF |
| 3381 | CAD ~ Intercept.Low + NTproBNP + PlGF + A8MPO |
| 3382 | CAD ~ Intercept.Low + NTproBNP + PlGF + A12TNF |
| 3383 | CAD ~ Intercept.Low + NTproBNP + A8MPO + A12TNF |
| 3384 | CAD ~ Intercept.Low + PlGF + A8MPO + A12TNF |
| 3385 | CAD ~ Intercept.Low + NTproBNP |
| 3386 | CAD ~ Intercept.Low + AdipA1 |
| 3387 | CAD ~ Intercept.Low + PlGF |
| 3388 | CAD ~ Intercept.Low + A8MPO |
| 3389 | CAD ~ Intercept.Low + A12TNF |
| 3390 | CAD ~ Intercept.Low + NTproBNP |
| 3391 | CAD ~ Intercept.Low + AdipA1 |
| 3392 | CAD ~ Intercept.Low + PlGF |
| 3393 | CAD ~ Intercept.Low + A8MPO |
| 3394 | CAD ~ Intercept.Low + A12TNF |
| 3395 | CAD ~ Intercept.Low + NTproBNP |
| 3396 | CAD ~ Intercept.Low + AdipA1 |
| 3397 | CAD ~ Intercept.Low + PlGF |
| 3398 | CAD ~ Intercept.Low + A8MPO |
| 3399 | CAD ~ Intercept.Low + A12TNF |
| 3400 | CAD ~ Intercept.Low + NTproBNP + PlGF |
| 3401 | CAD ~ Intercept.Low + AdipA1 + NTproBNP |
| 3402 | CAD ~ Intercept.Low + AdipA1 + PlGF |
| 3403 | CAD ~ Intercept.Low + AdipA1 + A8MPO |
| 3404 | CAD ~ Intercept.Low + AdipA1 + A12TNF |
| 3405 | CAD ~ Intercept.Low + NTproBNP + A8MPO |
| 3406 | CAD ~ Intercept.Low + NTproBNP + A12TNF |
| 3407 | CAD ~ Intercept.Low + PlGF + A8MPO |
| 3408 | CAD ~ Intercept.Low + PlGF + A12TNF |
| 3409 | CAD ~ Intercept.Low + A8MPO + A12TNF |
| 3410 | CAD ~ Intercept.Low + NTproBNP |
| 3411 | CAD ~ Intercept.Low + AdipA1 |
| 3412 | CAD ~ Intercept.Low + PlGF |
| 3413 | CAD ~ Intercept.Low + A8MPO |
| 3414 | CAD ~ Intercept.Low + A12TNF |
| 3415 | CAD ~ Intercept.Low + NTproBNP |
| 3416 | CAD ~ Intercept.Low + AdipA1 |
| 3417 | CAD ~ Intercept.Low + PlGF |
| 3418 | CAD ~ Intercept.Low + A8MPO |
| 3419 | CAD ~ Intercept.Low + A12TNF |
| 3420 | CAD ~ Intercept.Low + NTproBNP + PlGF |
| 3421 | CAD ~ Intercept.Low + AdipA1 + NTproBNP |
| 3422 | CAD ~ Intercept.Low + AdipA1 + PlGF |
| 3423 | CAD ~ Intercept.Low + AdipA1 + A8MPO |
| 3424 | CAD ~ Intercept.Low + AdipA1 + A12TNF |
| 3425 | CAD ~ Intercept.Low + NTproBNP + A8MPO |
| 3426 | CAD ~ Intercept.Low + NTproBNP + A12TNF |
| 3427 | CAD ~ Intercept.Low + PlGF + A8MPO |
| 3428 | CAD ~ Intercept.Low + PlGF + A12TNF |
| 3429 | CAD ~ Intercept.Low + A8MPO + A12TNF |
| 3430 | CAD ~ Intercept.Low + NTproBNP |
| 3431 | CAD ~ Intercept.Low + AdipA1 |
| 3432 | CAD ~ Intercept.Low + PlGF |
| 3433 | CAD ~ Intercept.Low + A8MPO |
| 3434 | CAD ~ Intercept.Low + A12TNF |
| 3435 | CAD ~ Intercept.Low + NTproBNP + PlGF |
| 3436 | CAD ~ Intercept.Low + AdipA1 + NTproBNP |
| 3437 | CAD ~ Intercept.Low + AdipA1 + PlGF |
| 3438 | CAD ~ Intercept.Low + AdipA1 + A8MPO |
| 3439 | CAD ~ Intercept.Low + AdipA1 + A12TNF |
| 3440 | CAD ~ Intercept.Low + NTproBNP + A8MPO |
| 3441 | CAD ~ Intercept.Low + NTproBNP + A12TNF |
| 3442 | CAD ~ Intercept.Low + PlGF + A8MPO |
| 3443 | CAD ~ Intercept.Low + PlGF + A12TNF |
| 3444 | CAD ~ Intercept.Low + A8MPO + A12TNF |
| 3445 | CAD ~ Intercept.Low + NTproBNP + PlGF |
| 3446 | CAD ~ Intercept.Low + AdipA1 + NTproBNP |
| 3447 | CAD ~ Intercept.Low + AdipA1 + PlGF |
| 3448 | CAD ~ Intercept.Low + AdipA1 + A8MPO |
| 3449 | CAD ~ Intercept.Low + AdipA1 + A12TNF |
| 3450 | CAD ~ Intercept.Low + NTproBNP + A8MPO |
| 3451 | CAD ~ Intercept.Low + NTproBNP + A12TNF |
| 3452 | CAD ~ Intercept.Low + PlGF + A8MPO |
| 3453 | CAD ~ Intercept.Low + PlGF + A12TNF |
| 3454 | CAD ~ Intercept.Low + A8MPO + A12TNF |
| 3455 | CAD ~ Intercept.Low + AdipA1 + NTproBNP + PlGF |
| 3456 | CAD ~ Intercept.Low + AdipA1 + NTproBNP + A8MPO |
| 3457 | CAD ~ Intercept.Low + AdipA1 + NTproBNP + A12TNF |
| 3458 | CAD ~ Intercept.Low + AdipA1 + PlGF + A8MPO |
| 3459 | CAD ~ Intercept.Low + AdipA1 + PlGF + A12TNF |
| 3460 | CAD ~ Intercept.Low + AdipA1 + A8MPO + A12TNF |
| 3461 | CAD ~ Intercept.Low + NTproBNP + PlGF + A8MPO |
| 3462 | CAD ~ Intercept.Low + NTproBNP + PlGF + A12TNF |
| 3463 | CAD ~ Intercept.Low + NTproBNP + A8MPO + A12TNF |
| 3464 | CAD ~ Intercept.Low + PlGF + A8MPO + A12TNF |
| 3465 | CAD ~ Intercept.Low + NTproBNP |
| 3466 | CAD ~ Intercept.Low + AdipA1 |
| 3467 | CAD ~ Intercept.Low + PlGF |
| 3468 | CAD ~ Intercept.Low + A8MPO |
| 3469 | CAD ~ Intercept.Low + A12TNF |
| 3470 | CAD ~ Intercept.Low + NTproBNP |
| 3471 | CAD ~ Intercept.Low + AdipA1 |
| 3472 | CAD ~ Intercept.Low + PlGF |
| 3473 | CAD ~ Intercept.Low + A8MPO |
| 3474 | CAD ~ Intercept.Low + A12TNF |
| 3475 | CAD ~ Intercept.Low + NTproBNP + PlGF |
| 3476 | CAD ~ Intercept.Low + AdipA1 + NTproBNP |
| 3477 | CAD ~ Intercept.Low + AdipA1 + PlGF |
| 3478 | CAD ~ Intercept.Low + AdipA1 + A8MPO |
| 3479 | CAD ~ Intercept.Low + AdipA1 + A12TNF |
| 3480 | CAD ~ Intercept.Low + NTproBNP + A8MPO |
| 3481 | CAD ~ Intercept.Low + NTproBNP + A12TNF |
| 3482 | CAD ~ Intercept.Low + PlGF + A8MPO |
| 3483 | CAD ~ Intercept.Low + PlGF + A12TNF |
| 3484 | CAD ~ Intercept.Low + A8MPO + A12TNF |
| 3485 | CAD ~ Intercept.Low + NTproBNP |
| 3486 | CAD ~ Intercept.Low + AdipA1 |
| 3487 | CAD ~ Intercept.Low + PlGF |
| 3488 | CAD ~ Intercept.Low + A8MPO |
| 3489 | CAD ~ Intercept.Low + A12TNF |
| 3490 | CAD ~ Intercept.Low + NTproBNP + PlGF |
| 3491 | CAD ~ Intercept.Low + AdipA1 + NTproBNP |
| 3492 | CAD ~ Intercept.Low + AdipA1 + PlGF |
| 3493 | CAD ~ Intercept.Low + AdipA1 + A8MPO |
| 3494 | CAD ~ Intercept.Low + AdipA1 + A12TNF |
| 3495 | CAD ~ Intercept.Low + NTproBNP + A8MPO |
| 3496 | CAD ~ Intercept.Low + NTproBNP + A12TNF |
| 3497 | CAD ~ Intercept.Low + PlGF + A8MPO |
| 3498 | CAD ~ Intercept.Low + PlGF + A12TNF |
| 3499 | CAD ~ Intercept.Low + A8MPO + A12TNF |
| 3500 | CAD ~ Intercept.Low + NTproBNP + PlGF |
| 3501 | CAD ~ Intercept.Low + AdipA1 + NTproBNP |
| 3502 | CAD ~ Intercept.Low + AdipA1 + PlGF |
| 3503 | CAD ~ Intercept.Low + AdipA1 + A8MPO |
| 3504 | CAD ~ Intercept.Low + AdipA1 + A12TNF |
| 3505 | CAD ~ Intercept.Low + NTproBNP + A8MPO |
| 3506 | CAD ~ Intercept.Low + NTproBNP + A12TNF |
| 3507 | CAD ~ Intercept.Low + PlGF + A8MPO |
| 3508 | CAD ~ Intercept.Low + PlGF + A12TNF |
| 3509 | CAD ~ Intercept.Low + A8MPO + A12TNF |
| 3510 | CAD ~ Intercept.Low + AdipA1 + NTproBNP + PlGF |
| 3511 | CAD ~ Intercept.Low + AdipA1 + NTproBNP + A8MPO |
| 3512 | CAD ~ Intercept.Low + AdipA1 + NTproBNP + A12TNF |
| 3513 | CAD ~ Intercept.Low + AdipA1 + PlGF + A8MPO |
| 3514 | CAD ~ Intercept.Low + AdipA1 + PlGF + A12TNF |
| 3515 | CAD ~ Intercept.Low + AdipA1 + A8MPO + A12TNF |
| 3516 | CAD ~ Intercept.Low + NTproBNP + PlGF + A8MPO |
| 3517 | CAD ~ Intercept.Low + NTproBNP + PlGF + A12TNF |
| 3518 | CAD ~ Intercept.Low + NTproBNP + A8MPO + A12TNF |
| 3519 | CAD ~ Intercept.Low + PlGF + A8MPO + A12TNF |
| 3520 | CAD ~ Intercept.Low + NTproBNP |
| 3521 | CAD ~ Intercept.Low + AdipA1 |
| 3522 | CAD ~ Intercept.Low + PlGF |
| 3523 | CAD ~ Intercept.Low + A8MPO |
| 3524 | CAD ~ Intercept.Low + A12TNF |
| 3525 | CAD ~ Intercept.Low + NTproBNP |
| 3526 | CAD ~ Intercept.Low + AdipA1 |
| 3527 | CAD ~ Intercept.Low + PlGF |
| 3528 | CAD ~ Intercept.Low + A8MPO |
| 3529 | CAD ~ Intercept.Low + A12TNF |
| 3530 | CAD ~ Intercept.Low + NTproBNP |
| 3531 | CAD ~ Intercept.Low + AdipA1 |
| 3532 | CAD ~ Intercept.Low + PlGF |
| 3533 | CAD ~ Intercept.Low + A8MPO |
| 3534 | CAD ~ Intercept.Low + A12TNF |
| 3535 | CAD ~ Intercept.Low + NTproBNP |
| 3536 | CAD ~ Intercept.Low + AdipA1 |
| 3537 | CAD ~ Intercept.Low + PlGF |
| 3538 | CAD ~ Intercept.Low + A8MPO |
| 3539 | CAD ~ Intercept.Low + A12TNF |
| 3540 | CAD ~ Intercept.Low + NTproBNP |
| 3541 | CAD ~ Intercept.Low + AdipA1 |
| 3542 | CAD ~ Intercept.Low + PlGF |
| 3543 | CAD ~ Intercept.Low + A8MPO |
| 3544 | CAD ~ Intercept.Low + A12TNF |
| 3545 | CAD ~ Intercept.Low + NTproBNP + PlGF |
| 3546 | CAD ~ Intercept.Low + AdipA1 + NTproBNP |
| 3547 | CAD ~ Intercept.Low + AdipA1 + PlGF |
| 3548 | CAD ~ Intercept.Low + AdipA1 + A8MPO |
| 3549 | CAD ~ Intercept.Low + AdipA1 + A12TNF |
| 3550 | CAD ~ Intercept.Low + NTproBNP + A8MPO |
| 3551 | CAD ~ Intercept.Low + NTproBNP + A12TNF |
| 3552 | CAD ~ Intercept.Low + PlGF + A8MPO |
| 3553 | CAD ~ Intercept.Low + PlGF + A12TNF |
| 3554 | CAD ~ Intercept.Low + A8MPO + A12TNF |
| 3555 | CAD ~ Intercept.Low + NTproBNP |
| 3556 | CAD ~ Intercept.Low + AdipA1 |
| 3557 | CAD ~ Intercept.Low + PlGF |
| 3558 | CAD ~ Intercept.Low + A8MPO |
| 3559 | CAD ~ Intercept.Low + A12TNF |
| 3560 | CAD ~ Intercept.Low + NTproBNP |
| 3561 | CAD ~ Intercept.Low + AdipA1 |
| 3562 | CAD ~ Intercept.Low + PlGF |
| 3563 | CAD ~ Intercept.Low + A8MPO |
| 3564 | CAD ~ Intercept.Low + A12TNF |
| 3565 | CAD ~ Intercept.Low + NTproBNP |
| 3566 | CAD ~ Intercept.Low + AdipA1 |
| 3567 | CAD ~ Intercept.Low + PlGF |
| 3568 | CAD ~ Intercept.Low + A8MPO |
| 3569 | CAD ~ Intercept.Low + A12TNF |
| 3570 | CAD ~ Intercept.Low + NTproBNP |
| 3571 | CAD ~ Intercept.Low + AdipA1 |
| 3572 | CAD ~ Intercept.Low + PlGF |
| 3573 | CAD ~ Intercept.Low + A8MPO |
| 3574 | CAD ~ Intercept.Low + A12TNF |
| 3575 | CAD ~ Intercept.Low + NTproBNP + PlGF |
| 3576 | CAD ~ Intercept.Low + AdipA1 + NTproBNP |
| 3577 | CAD ~ Intercept.Low + AdipA1 + PlGF |
| 3578 | CAD ~ Intercept.Low + AdipA1 + A8MPO |
| 3579 | CAD ~ Intercept.Low + AdipA1 + A12TNF |
| 3580 | CAD ~ Intercept.Low + NTproBNP + A8MPO |
| 3581 | CAD ~ Intercept.Low + NTproBNP + A12TNF |
| 3582 | CAD ~ Intercept.Low + PlGF + A8MPO |
| 3583 | CAD ~ Intercept.Low + PlGF + A12TNF |
| 3584 | CAD ~ Intercept.Low + A8MPO + A12TNF |
| 3585 | CAD ~ Intercept.Low + NTproBNP |
| 3586 | CAD ~ Intercept.Low + AdipA1 |
| 3587 | CAD ~ Intercept.Low + PlGF |
| 3588 | CAD ~ Intercept.Low + A8MPO |
| 3589 | CAD ~ Intercept.Low + A12TNF |
| 3590 | CAD ~ Intercept.Low + NTproBNP |
| 3591 | CAD ~ Intercept.Low + AdipA1 |
| 3592 | CAD ~ Intercept.Low + PlGF |
| 3593 | CAD ~ Intercept.Low + A8MPO |
| 3594 | CAD ~ Intercept.Low + A12TNF |
| 3595 | CAD ~ Intercept.Low + NTproBNP |
| 3596 | CAD ~ Intercept.Low + AdipA1 |
| 3597 | CAD ~ Intercept.Low + PlGF |
| 3598 | CAD ~ Intercept.Low + A8MPO |
| 3599 | CAD ~ Intercept.Low + A12TNF |
| 3600 | CAD ~ Intercept.Low + NTproBNP + PlGF |
| 3601 | CAD ~ Intercept.Low + AdipA1 + NTproBNP |
| 3602 | CAD ~ Intercept.Low + AdipA1 + PlGF |
| 3603 | CAD ~ Intercept.Low + AdipA1 + A8MPO |
| 3604 | CAD ~ Intercept.Low + AdipA1 + A12TNF |
| 3605 | CAD ~ Intercept.Low + NTproBNP + A8MPO |
| 3606 | CAD ~ Intercept.Low + NTproBNP + A12TNF |
| 3607 | CAD ~ Intercept.Low + PlGF + A8MPO |
| 3608 | CAD ~ Intercept.Low + PlGF + A12TNF |
| 3609 | CAD ~ Intercept.Low + A8MPO + A12TNF |
| 3610 | CAD ~ Intercept.Low + NTproBNP |
| 3611 | CAD ~ Intercept.Low + AdipA1 |
| 3612 | CAD ~ Intercept.Low + PlGF |
| 3613 | CAD ~ Intercept.Low + A8MPO |
| 3614 | CAD ~ Intercept.Low + A12TNF |
| 3615 | CAD ~ Intercept.Low + NTproBNP |
| 3616 | CAD ~ Intercept.Low + AdipA1 |
| 3617 | CAD ~ Intercept.Low + PlGF |
| 3618 | CAD ~ Intercept.Low + A8MPO |
| 3619 | CAD ~ Intercept.Low + A12TNF |
| 3620 | CAD ~ Intercept.Low + NTproBNP + PlGF |
| 3621 | CAD ~ Intercept.Low + AdipA1 + NTproBNP |
| 3622 | CAD ~ Intercept.Low + AdipA1 + PlGF |
| 3623 | CAD ~ Intercept.Low + AdipA1 + A8MPO |
| 3624 | CAD ~ Intercept.Low + AdipA1 + A12TNF |
| 3625 | CAD ~ Intercept.Low + NTproBNP + A8MPO |
| 3626 | CAD ~ Intercept.Low + NTproBNP + A12TNF |
| 3627 | CAD ~ Intercept.Low + PlGF + A8MPO |
| 3628 | CAD ~ Intercept.Low + PlGF + A12TNF |
| 3629 | CAD ~ Intercept.Low + A8MPO + A12TNF |
| 3630 | CAD ~ Intercept.Low + NTproBNP |
| 3631 | CAD ~ Intercept.Low + AdipA1 |
| 3632 | CAD ~ Intercept.Low + PlGF |
| 3633 | CAD ~ Intercept.Low + A8MPO |
| 3634 | CAD ~ Intercept.Low + A12TNF |
| 3635 | CAD ~ Intercept.Low + NTproBNP + PlGF |
| 3636 | CAD ~ Intercept.Low + AdipA1 + NTproBNP |
| 3637 | CAD ~ Intercept.Low + AdipA1 + PlGF |
| 3638 | CAD ~ Intercept.Low + AdipA1 + A8MPO |
| 3639 | CAD ~ Intercept.Low + AdipA1 + A12TNF |
| 3640 | CAD ~ Intercept.Low + NTproBNP + A8MPO |
| 3641 | CAD ~ Intercept.Low + NTproBNP + A12TNF |
| 3642 | CAD ~ Intercept.Low + PlGF + A8MPO |
| 3643 | CAD ~ Intercept.Low + PlGF + A12TNF |
| 3644 | CAD ~ Intercept.Low + A8MPO + A12TNF |
| 3645 | CAD ~ Intercept.Low + NTproBNP + PlGF |
| 3646 | CAD ~ Intercept.Low + AdipA1 + NTproBNP |
| 3647 | CAD ~ Intercept.Low + AdipA1 + PlGF |
| 3648 | CAD ~ Intercept.Low + AdipA1 + A8MPO |
| 3649 | CAD ~ Intercept.Low + AdipA1 + A12TNF |
| 3650 | CAD ~ Intercept.Low + NTproBNP + A8MPO |
| 3651 | CAD ~ Intercept.Low + NTproBNP + A12TNF |
| 3652 | CAD ~ Intercept.Low + PlGF + A8MPO |
| 3653 | CAD ~ Intercept.Low + PlGF + A12TNF |
| 3654 | CAD ~ Intercept.Low + A8MPO + A12TNF |
| 3655 | CAD ~ Intercept.Low + AdipA1 + NTproBNP + PlGF |
| 3656 | CAD ~ Intercept.Low + AdipA1 + NTproBNP + A8MPO |
| 3657 | CAD ~ Intercept.Low + AdipA1 + NTproBNP + A12TNF |
| 3658 | CAD ~ Intercept.Low + AdipA1 + PlGF + A8MPO |
| 3659 | CAD ~ Intercept.Low + AdipA1 + PlGF + A12TNF |
| 3660 | CAD ~ Intercept.Low + AdipA1 + A8MPO + A12TNF |
| 3661 | CAD ~ Intercept.Low + NTproBNP + PlGF + A8MPO |
| 3662 | CAD ~ Intercept.Low + NTproBNP + PlGF + A12TNF |
| 3663 | CAD ~ Intercept.Low + NTproBNP + A8MPO + A12TNF |
| 3664 | CAD ~ Intercept.Low + PlGF + A8MPO + A12TNF |
| 3665 | CAD ~ Intercept.Low + NTproBNP |
| 3666 | CAD ~ Intercept.Low + AdipA1 |
| 3667 | CAD ~ Intercept.Low + PlGF |
| 3668 | CAD ~ Intercept.Low + A8MPO |
| 3669 | CAD ~ Intercept.Low + A12TNF |
| 3670 | CAD ~ Intercept.Low + NTproBNP + PlGF |
| 3671 | CAD ~ Intercept.Low + AdipA1 + NTproBNP |
| 3672 | CAD ~ Intercept.Low + AdipA1 + PlGF |
| 3673 | CAD ~ Intercept.Low + AdipA1 + A8MPO |
| 3674 | CAD ~ Intercept.Low + AdipA1 + A12TNF |
| 3675 | CAD ~ Intercept.Low + NTproBNP + A8MPO |
| 3676 | CAD ~ Intercept.Low + NTproBNP + A12TNF |
| 3677 | CAD ~ Intercept.Low + PlGF + A8MPO |
| 3678 | CAD ~ Intercept.Low + PlGF + A12TNF |
| 3679 | CAD ~ Intercept.Low + A8MPO + A12TNF |
| 3680 | CAD ~ Intercept.Low + NTproBNP + PlGF |
| 3681 | CAD ~ Intercept.Low + AdipA1 + NTproBNP |
| 3682 | CAD ~ Intercept.Low + AdipA1 + PlGF |
| 3683 | CAD ~ Intercept.Low + AdipA1 + A8MPO |
| 3684 | CAD ~ Intercept.Low + AdipA1 + A12TNF |
| 3685 | CAD ~ Intercept.Low + NTproBNP + A8MPO |
| 3686 | CAD ~ Intercept.Low + NTproBNP + A12TNF |
| 3687 | CAD ~ Intercept.Low + PlGF + A8MPO |
| 3688 | CAD ~ Intercept.Low + PlGF + A12TNF |
| 3689 | CAD ~ Intercept.Low + A8MPO + A12TNF |
| 3690 | CAD ~ Intercept.Low + AdipA1 + NTproBNP + PlGF |
| 3691 | CAD ~ Intercept.Low + AdipA1 + NTproBNP + A8MPO |
| 3692 | CAD ~ Intercept.Low + AdipA1 + NTproBNP + A12TNF |
| 3693 | CAD ~ Intercept.Low + AdipA1 + PlGF + A8MPO |
| 3694 | CAD ~ Intercept.Low + AdipA1 + PlGF + A12TNF |
| 3695 | CAD ~ Intercept.Low + AdipA1 + A8MPO + A12TNF |
| 3696 | CAD ~ Intercept.Low + NTproBNP + PlGF + A8MPO |
| 3697 | CAD ~ Intercept.Low + NTproBNP + PlGF + A12TNF |
| 3698 | CAD ~ Intercept.Low + NTproBNP + A8MPO + A12TNF |
| 3699 | CAD ~ Intercept.Low + PlGF + A8MPO + A12TNF |
| 3700 | CAD ~ Intercept.Low + NTproBNP + PlGF |
| 3701 | CAD ~ Intercept.Low + AdipA1 + NTproBNP |
| 3702 | CAD ~ Intercept.Low + AdipA1 + PlGF |
| 3703 | CAD ~ Intercept.Low + AdipA1 + A8MPO |
| 3704 | CAD ~ Intercept.Low + AdipA1 + A12TNF |
| 3705 | CAD ~ Intercept.Low + NTproBNP + A8MPO |
| 3706 | CAD ~ Intercept.Low + NTproBNP + A12TNF |
| 3707 | CAD ~ Intercept.Low + PlGF + A8MPO |
| 3708 | CAD ~ Intercept.Low + PlGF + A12TNF |
| 3709 | CAD ~ Intercept.Low + A8MPO + A12TNF |
| 3710 | CAD ~ Intercept.Low + AdipA1 + NTproBNP + PlGF |
| 3711 | CAD ~ Intercept.Low + AdipA1 + NTproBNP + A8MPO |
| 3712 | CAD ~ Intercept.Low + AdipA1 + NTproBNP + A12TNF |
| 3713 | CAD ~ Intercept.Low + AdipA1 + PlGF + A8MPO |
| 3714 | CAD ~ Intercept.Low + AdipA1 + PlGF + A12TNF |
| 3715 | CAD ~ Intercept.Low + AdipA1 + A8MPO + A12TNF |
| 3716 | CAD ~ Intercept.Low + NTproBNP + PlGF + A8MPO |
| 3717 | CAD ~ Intercept.Low + NTproBNP + PlGF + A12TNF |
| 3718 | CAD ~ Intercept.Low + NTproBNP + A8MPO + A12TNF |
| 3719 | CAD ~ Intercept.Low + PlGF + A8MPO + A12TNF |
| 3720 | CAD ~ Intercept.Low + AdipA1 + NTproBNP + PlGF |
| 3721 | CAD ~ Intercept.Low + AdipA1 + NTproBNP + A8MPO |
| 3722 | CAD ~ Intercept.Low + AdipA1 + NTproBNP + A12TNF |
| 3723 | CAD ~ Intercept.Low + AdipA1 + PlGF + A8MPO |
| 3724 | CAD ~ Intercept.Low + AdipA1 + PlGF + A12TNF |
| 3725 | CAD ~ Intercept.Low + AdipA1 + A8MPO + A12TNF |
| 3726 | CAD ~ Intercept.Low + NTproBNP + PlGF + A8MPO |
| 3727 | CAD ~ Intercept.Low + NTproBNP + PlGF + A12TNF |
| 3728 | CAD ~ Intercept.Low + NTproBNP + A8MPO + A12TNF |
| 3729 | CAD ~ Intercept.Low + PlGF + A8MPO + A12TNF |
| 3730 | NA |
| 3731 | NA |
| 3732 | NA |
| 3733 | NA |
| 3734 | NA |
| 3735 | NA |
| 3736 | NA |
| 3737 | CAD ~ Intercept.Low + AdipA1 + NTproBNP + PlGF + A8MPO |
| 3738 | CAD ~ Intercept.Low + AdipA1 + NTproBNP + PlGF + A12TNF |
| 3739 | CAD ~ Intercept.Low + AdipA1 + NTproBNP + A8MPO + A12TNF |
| 3740 | CAD ~ Intercept.Low + AdipA1 + PlGF + A8MPO + A12TNF |
| 3741 | CAD ~ Intercept.Low + NTproBNP + PlGF + A8MPO + A12TNF |
| 3742 | CAD ~ Intercept.Low + NTproBNP |
| 3743 | CAD ~ Intercept.Low + AdipA1 |
| 3744 | CAD ~ Intercept.Low + PlGF |
| 3745 | CAD ~ Intercept.Low + A8MPO |
| 3746 | CAD ~ Intercept.Low + A12TNF |
| 3747 | CAD ~ Intercept.Low + NTproBNP |
| 3748 | CAD ~ Intercept.Low + AdipA1 |
| 3749 | CAD ~ Intercept.Low + PlGF |
| 3750 | CAD ~ Intercept.Low + A8MPO |
| 3751 | CAD ~ Intercept.Low + A12TNF |
| 3752 | CAD ~ Intercept.Low + NTproBNP |
| 3753 | CAD ~ Intercept.Low + AdipA1 |
| 3754 | CAD ~ Intercept.Low + PlGF |
| 3755 | CAD ~ Intercept.Low + A8MPO |
| 3756 | CAD ~ Intercept.Low + A12TNF |
| 3757 | CAD ~ Intercept.Low + NTproBNP |
| 3758 | CAD ~ Intercept.Low + AdipA1 |
| 3759 | CAD ~ Intercept.Low + PlGF |
| 3760 | CAD ~ Intercept.Low + A8MPO |
| 3761 | CAD ~ Intercept.Low + A12TNF |
| 3762 | CAD ~ Intercept.Low + NTproBNP |
| 3763 | CAD ~ Intercept.Low + AdipA1 |
| 3764 | CAD ~ Intercept.Low + PlGF |
| 3765 | CAD ~ Intercept.Low + A8MPO |
| 3766 | CAD ~ Intercept.Low + A12TNF |
| 3767 | CAD ~ Intercept.Low + NTproBNP + PlGF |
| 3768 | CAD ~ Intercept.Low + AdipA1 + NTproBNP |
| 3769 | CAD ~ Intercept.Low + AdipA1 + PlGF |
| 3770 | CAD ~ Intercept.Low + AdipA1 + A8MPO |
| 3771 | CAD ~ Intercept.Low + AdipA1 + A12TNF |
| 3772 | CAD ~ Intercept.Low + NTproBNP + A8MPO |
| 3773 | CAD ~ Intercept.Low + NTproBNP + A12TNF |
| 3774 | CAD ~ Intercept.Low + PlGF + A8MPO |
| 3775 | CAD ~ Intercept.Low + PlGF + A12TNF |
| 3776 | CAD ~ Intercept.Low + A8MPO + A12TNF |
| 3777 | CAD ~ Intercept.Low + NTproBNP |
| 3778 | CAD ~ Intercept.Low + AdipA1 |
| 3779 | CAD ~ Intercept.Low + PlGF |
| 3780 | CAD ~ Intercept.Low + A8MPO |
| 3781 | CAD ~ Intercept.Low + A12TNF |
| 3782 | CAD ~ Intercept.Low + NTproBNP |
| 3783 | CAD ~ Intercept.Low + AdipA1 |
| 3784 | CAD ~ Intercept.Low + PlGF |
| 3785 | CAD ~ Intercept.Low + A8MPO |
| 3786 | CAD ~ Intercept.Low + A12TNF |
| 3787 | CAD ~ Intercept.Low + NTproBNP |
| 3788 | CAD ~ Intercept.Low + AdipA1 |
| 3789 | CAD ~ Intercept.Low + PlGF |
| 3790 | CAD ~ Intercept.Low + A8MPO |
| 3791 | CAD ~ Intercept.Low + A12TNF |
| 3792 | CAD ~ Intercept.Low + NTproBNP |
| 3793 | CAD ~ Intercept.Low + AdipA1 |
| 3794 | CAD ~ Intercept.Low + PlGF |
| 3795 | CAD ~ Intercept.Low + A8MPO |
| 3796 | CAD ~ Intercept.Low + A12TNF |
| 3797 | CAD ~ Intercept.Low + NTproBNP + PlGF |
| 3798 | CAD ~ Intercept.Low + AdipA1 + NTproBNP |
| 3799 | CAD ~ Intercept.Low + AdipA1 + PlGF |
| 3800 | CAD ~ Intercept.Low + AdipA1 + A8MPO |
| 3801 | CAD ~ Intercept.Low + AdipA1 + A12TNF |
| 3802 | CAD ~ Intercept.Low + NTproBNP + A8MPO |
| 3803 | CAD ~ Intercept.Low + NTproBNP + A12TNF |
| 3804 | CAD ~ Intercept.Low + PlGF + A8MPO |
| 3805 | CAD ~ Intercept.Low + PlGF + A12TNF |
| 3806 | CAD ~ Intercept.Low + A8MPO + A12TNF |
| 3807 | CAD ~ Intercept.Low + NTproBNP |
| 3808 | CAD ~ Intercept.Low + AdipA1 |
| 3809 | CAD ~ Intercept.Low + PlGF |
| 3810 | CAD ~ Intercept.Low + A8MPO |
| 3811 | CAD ~ Intercept.Low + A12TNF |
| 3812 | CAD ~ Intercept.Low + NTproBNP |
| 3813 | CAD ~ Intercept.Low + AdipA1 |
| 3814 | CAD ~ Intercept.Low + PlGF |
| 3815 | CAD ~ Intercept.Low + A8MPO |
| 3816 | CAD ~ Intercept.Low + A12TNF |
| 3817 | CAD ~ Intercept.Low + NTproBNP |
| 3818 | CAD ~ Intercept.Low + AdipA1 |
| 3819 | CAD ~ Intercept.Low + PlGF |
| 3820 | CAD ~ Intercept.Low + A8MPO |
| 3821 | CAD ~ Intercept.Low + A12TNF |
| 3822 | CAD ~ Intercept.Low + NTproBNP + PlGF |
| 3823 | CAD ~ Intercept.Low + AdipA1 + NTproBNP |
| 3824 | CAD ~ Intercept.Low + AdipA1 + PlGF |
| 3825 | CAD ~ Intercept.Low + AdipA1 + A8MPO |
| 3826 | CAD ~ Intercept.Low + AdipA1 + A12TNF |
| 3827 | CAD ~ Intercept.Low + NTproBNP + A8MPO |
| 3828 | CAD ~ Intercept.Low + NTproBNP + A12TNF |
| 3829 | CAD ~ Intercept.Low + PlGF + A8MPO |
| 3830 | CAD ~ Intercept.Low + PlGF + A12TNF |
| 3831 | CAD ~ Intercept.Low + A8MPO + A12TNF |
| 3832 | CAD ~ Intercept.Low + NTproBNP |
| 3833 | CAD ~ Intercept.Low + AdipA1 |
| 3834 | CAD ~ Intercept.Low + PlGF |
| 3835 | CAD ~ Intercept.Low + A8MPO |
| 3836 | CAD ~ Intercept.Low + A12TNF |
| 3837 | CAD ~ Intercept.Low + NTproBNP |
| 3838 | CAD ~ Intercept.Low + AdipA1 |
| 3839 | CAD ~ Intercept.Low + PlGF |
| 3840 | CAD ~ Intercept.Low + A8MPO |
| 3841 | CAD ~ Intercept.Low + A12TNF |
| 3842 | CAD ~ Intercept.Low + NTproBNP |
| 3843 | CAD ~ Intercept.Low + AdipA1 |
| 3844 | CAD ~ Intercept.Low + PlGF |
| 3845 | CAD ~ Intercept.Low + A8MPO |
| 3846 | CAD ~ Intercept.Low + A12TNF |
| 3847 | CAD ~ Intercept.Low + NTproBNP |
| 3848 | CAD ~ Intercept.Low + AdipA1 |
| 3849 | CAD ~ Intercept.Low + PlGF |
| 3850 | CAD ~ Intercept.Low + A8MPO |
| 3851 | CAD ~ Intercept.Low + A12TNF |
| 3852 | CAD ~ Intercept.Low + NTproBNP |
| 3853 | CAD ~ Intercept.Low + AdipA1 |
| 3854 | CAD ~ Intercept.Low + PlGF |
| 3855 | CAD ~ Intercept.Low + A8MPO |
| 3856 | CAD ~ Intercept.Low + A12TNF |
| 3857 | CAD ~ Intercept.Low + NTproBNP |
| 3858 | CAD ~ Intercept.Low + AdipA1 |
| 3859 | CAD ~ Intercept.Low + PlGF |
| 3860 | CAD ~ Intercept.Low + A8MPO |
| 3861 | CAD ~ Intercept.Low + A12TNF |
| 3862 | CAD ~ Intercept.Low + NTproBNP + PlGF |
| 3863 | CAD ~ Intercept.Low + AdipA1 + NTproBNP |
| 3864 | CAD ~ Intercept.Low + AdipA1 + PlGF |
| 3865 | CAD ~ Intercept.Low + AdipA1 + A8MPO |
| 3866 | CAD ~ Intercept.Low + AdipA1 + A12TNF |
| 3867 | CAD ~ Intercept.Low + NTproBNP + A8MPO |
| 3868 | CAD ~ Intercept.Low + NTproBNP + A12TNF |
| 3869 | CAD ~ Intercept.Low + PlGF + A8MPO |
| 3870 | CAD ~ Intercept.Low + PlGF + A12TNF |
| 3871 | CAD ~ Intercept.Low + A8MPO + A12TNF |
| 3872 | CAD ~ Intercept.Low + NTproBNP |
| 3873 | CAD ~ Intercept.Low + AdipA1 |
| 3874 | CAD ~ Intercept.Low + PlGF |
| 3875 | CAD ~ Intercept.Low + A8MPO |
| 3876 | CAD ~ Intercept.Low + A12TNF |
| 3877 | CAD ~ Intercept.Low + NTproBNP |
| 3878 | CAD ~ Intercept.Low + AdipA1 |
| 3879 | CAD ~ Intercept.Low + PlGF |
| 3880 | CAD ~ Intercept.Low + A8MPO |
| 3881 | CAD ~ Intercept.Low + A12TNF |
| 3882 | CAD ~ Intercept.Low + NTproBNP + PlGF |
| 3883 | CAD ~ Intercept.Low + AdipA1 + NTproBNP |
| 3884 | CAD ~ Intercept.Low + AdipA1 + PlGF |
| 3885 | CAD ~ Intercept.Low + AdipA1 + A8MPO |
| 3886 | CAD ~ Intercept.Low + AdipA1 + A12TNF |
| 3887 | CAD ~ Intercept.Low + NTproBNP + A8MPO |
| 3888 | CAD ~ Intercept.Low + NTproBNP + A12TNF |
| 3889 | CAD ~ Intercept.Low + PlGF + A8MPO |
| 3890 | CAD ~ Intercept.Low + PlGF + A12TNF |
| 3891 | CAD ~ Intercept.Low + A8MPO + A12TNF |
| 3892 | CAD ~ Intercept.Low + NTproBNP |
| 3893 | CAD ~ Intercept.Low + AdipA1 |
| 3894 | CAD ~ Intercept.Low + PlGF |
| 3895 | CAD ~ Intercept.Low + A8MPO |
| 3896 | CAD ~ Intercept.Low + A12TNF |
| 3897 | CAD ~ Intercept.Low + NTproBNP + PlGF |
| 3898 | CAD ~ Intercept.Low + AdipA1 + NTproBNP |
| 3899 | CAD ~ Intercept.Low + AdipA1 + PlGF |
| 3900 | CAD ~ Intercept.Low + AdipA1 + A8MPO |
| 3901 | CAD ~ Intercept.Low + AdipA1 + A12TNF |
| 3902 | CAD ~ Intercept.Low + NTproBNP + A8MPO |
| 3903 | CAD ~ Intercept.Low + NTproBNP + A12TNF |
| 3904 | CAD ~ Intercept.Low + PlGF + A8MPO |
| 3905 | CAD ~ Intercept.Low + PlGF + A12TNF |
| 3906 | CAD ~ Intercept.Low + A8MPO + A12TNF |
| 3907 | CAD ~ Intercept.Low + NTproBNP + PlGF |
| 3908 | CAD ~ Intercept.Low + AdipA1 + NTproBNP |
| 3909 | CAD ~ Intercept.Low + AdipA1 + PlGF |
| 3910 | CAD ~ Intercept.Low + AdipA1 + A8MPO |
| 3911 | CAD ~ Intercept.Low + AdipA1 + A12TNF |
| 3912 | CAD ~ Intercept.Low + NTproBNP + A8MPO |
| 3913 | CAD ~ Intercept.Low + NTproBNP + A12TNF |
| 3914 | CAD ~ Intercept.Low + PlGF + A8MPO |
| 3915 | CAD ~ Intercept.Low + PlGF + A12TNF |
| 3916 | CAD ~ Intercept.Low + A8MPO + A12TNF |
| 3917 | NA |
| 3918 | NA |
| 3919 | NA |
| 3920 | NA |
| 3921 | NA |
| 3922 | NA |
| 3923 | NA |
| 3924 | NA |
| 3925 | NA |
| 3926 | NA |
| 3927 | NA |
| 3928 | NA |
| 3929 | NA |
| 3930 | NA |
| 3931 | NA |
| 3932 | NA |
| 3933 | NA |
| 3934 | NA |
| 3935 | NA |
| 3936 | NA |
| 3937 | NA |
| 3938 | CAD ~ Intercept.Low + AdipA1 + NTproBNP + PlGF |
| 3939 | CAD ~ Intercept.Low + AdipA1 + NTproBNP + A8MPO |
| 3940 | CAD ~ Intercept.Low + AdipA1 + NTproBNP + A12TNF |
| 3941 | CAD ~ Intercept.Low + AdipA1 + PlGF + A8MPO |
| 3942 | CAD ~ Intercept.Low + AdipA1 + PlGF + A12TNF |
| 3943 | CAD ~ Intercept.Low + AdipA1 + A8MPO + A12TNF |
| 3944 | CAD ~ Intercept.Low + NTproBNP + PlGF + A8MPO |
| 3945 | CAD ~ Intercept.Low + NTproBNP + PlGF + A12TNF |
| 3946 | CAD ~ Intercept.Low + NTproBNP + A8MPO + A12TNF |
| 3947 | CAD ~ Intercept.Low + PlGF + A8MPO + A12TNF |
| 3948 | CAD ~ Intercept.Low + NTproBNP |
| 3949 | CAD ~ Intercept.Low + AdipA1 |
| 3950 | CAD ~ Intercept.Low + PlGF |
| 3951 | CAD ~ Intercept.Low + A8MPO |
| 3952 | CAD ~ Intercept.Low + A12TNF |
| 3953 | CAD ~ Intercept.Low + NTproBNP |
| 3954 | CAD ~ Intercept.Low + AdipA1 |
| 3955 | CAD ~ Intercept.Low + PlGF |
| 3956 | CAD ~ Intercept.Low + A8MPO |
| 3957 | CAD ~ Intercept.Low + A12TNF |
| 3958 | CAD ~ Intercept.Low + NTproBNP |
| 3959 | CAD ~ Intercept.Low + AdipA1 |
| 3960 | CAD ~ Intercept.Low + PlGF |
| 3961 | CAD ~ Intercept.Low + A8MPO |
| 3962 | CAD ~ Intercept.Low + A12TNF |
| 3963 | CAD ~ Intercept.Low + NTproBNP |
| 3964 | CAD ~ Intercept.Low + AdipA1 |
| 3965 | CAD ~ Intercept.Low + PlGF |
| 3966 | CAD ~ Intercept.Low + A8MPO |
| 3967 | CAD ~ Intercept.Low + A12TNF |
| 3968 | CAD ~ Intercept.Low + NTproBNP |
| 3969 | CAD ~ Intercept.Low + AdipA1 |
| 3970 | CAD ~ Intercept.Low + PlGF |
| 3971 | CAD ~ Intercept.Low + A8MPO |
| 3972 | CAD ~ Intercept.Low + A12TNF |
| 3973 | CAD ~ Intercept.Low + NTproBNP |
| 3974 | CAD ~ Intercept.Low + AdipA1 |
| 3975 | CAD ~ Intercept.Low + PlGF |
| 3976 | CAD ~ Intercept.Low + A8MPO |
| 3977 | CAD ~ Intercept.Low + A12TNF |
| 3978 | CAD ~ Intercept.Low + NTproBNP |
| 3979 | CAD ~ Intercept.Low + AdipA1 |
| 3980 | CAD ~ Intercept.Low + PlGF |
| 3981 | CAD ~ Intercept.Low + A8MPO |
| 3982 | CAD ~ Intercept.Low + A12TNF |
| 3983 | NA |
| 3984 | NA |
| 3985 | NA |
| 3986 | NA |
| 3987 | NA |
| 3988 | NA |
| 3989 | NA |
| 3990 | NA |
| 3991 | NA |
| 3992 | NA |
| 3993 | NA |
| 3994 | NA |
| 3995 | NA |
| 3996 | NA |
| 3997 | NA |
| 3998 | NA |
| 3999 | NA |
| 4000 | NA |
| 4001 | NA |
| 4002 | NA |
| 4003 | NA |
| 4004 | NA |
| 4005 | NA |
| 4006 | NA |
| 4007 | NA |
| 4008 | NA |
| 4009 | NA |
| 4010 | NA |
| 4011 | NA |
| 4012 | NA |
| 4013 | NA |
| 4014 | NA |
| 4015 | NA |
| 4016 | NA |
| 4017 | NA |
| 4018 | CAD ~ Intercept.Low + NTproBNP + PlGF |
| 4019 | CAD ~ Intercept.Low + NTproBNP + A8MPO |
| 4020 | CAD ~ Intercept.Low + NTproBNP + A12TNF |
| 4021 | CAD ~ Intercept.Low + PlGF + A8MPO |
| 4022 | CAD ~ Intercept.Low + PlGF + A12TNF |
| 4023 | CAD ~ Intercept.Low + A8MPO + A12TNF |
| 4024 | CAD ~ Intercept.Low + AdipA1 + NTproBNP |
| 4025 | CAD ~ Intercept.Low + AdipA1 + PlGF |
| 4026 | CAD ~ Intercept.Low + AdipA1 + A8MPO |
| 4027 | CAD ~ Intercept.Low + AdipA1 + A12TNF |
| 4028 | NA |
| 4029 | NA |
| 4030 | NA |
| 4031 | NA |
| 4032 | NA |
| 4033 | NA |
| 4034 | NA |
| 4035 | NA |
| 4036 | NA |
| 4037 | NA |
| 4038 | NA |
| 4039 | NA |
| 4040 | NA |
| 4041 | NA |
| 4042 | NA |
| 4043 | NA |
| 4044 | NA |
| 4045 | NA |
| 4046 | NA |
| 4047 | NA |
| 4048 | NA |
| 4049 | NA |
| 4050 | NA |
| 4051 | NA |
| 4052 | NA |
| 4053 | NA |
| 4054 | NA |
| 4055 | NA |
| 4056 | NA |
| 4057 | NA |
| 4058 | NA |
| 4059 | NA |
| 4060 | NA |
| 4061 | NA |
| 4062 | NA |
| 4063 | CAD ~ Intercept.Low + AdipA1 |
| 4064 | CAD ~ Intercept.Low + NTproBNP |
| 4065 | CAD ~ Intercept.Low + PlGF |
| 4066 | CAD ~ Intercept.Low + A8MPO |
| 4067 | CAD ~ Intercept.Low + A12TNF |
| 4068 | NA |
| 4069 | NA |
| 4070 | NA |
| 4071 | NA |
| 4072 | NA |
| 4073 | NA |
| 4074 | NA |
| 4075 | NA |
| 4076 | NA |
| 4077 | NA |
| 4078 | NA |
| 4079 | NA |
| 4080 | NA |
| 4081 | NA |
| 4082 | NA |
| 4083 | NA |
| 4084 | NA |
| 4085 | NA |
| 4086 | NA |
| 4087 | NA |
| 4088 | NA |
| 4089 | NA |
| 4090 | NA |
| 4091 | NA |
| 4092 | NA |
| 4093 | NA |
| 4094 | NA |
| 4095 | NA |

TABLE 26B

| | Intercept.Hiqh | Beta.SexMale | Beta.Age | Beta.APOB | Beta.corin | Beta.HSP70 | Beta.RBP4 | Beta.SERPINA12 | Beta.LowLevel |
|---|---|---|---|---|---|---|---|---|---|
| 1 | −5.29177 | 1.345188 | 0.069961 | 0.261727 | 0.029239 | −0.12978 | −0.05482 | −0.20628 | 0.760096 |
| 2 | −3.68958 | 0 | 0.057643 | 0.290584 | 0.100058 | −0.09737 | −0.06544 | −0.21635 | 0.868073 |
| 3 | −5.35257 | 1.35256 | 0.070578 | 0.256481 | 0.043435 | −0.13515 | −0.05432 | −0.17998 | 0.734446 |
| 4 | −5.33156 | 1.370597 | 0.070232 | 0.254283 | 0.026935 | −0.10443 | −0.05756 | −0.20745 | 0.748267 |
| 5 | −5.3061 | 1.352024 | 0.069948 | 0.268648 | 0.041034 | −0.12817 | −0.05834 | −0.20277 | 0.749805 |
| 6 | −5.5685 | 1.325938 | 0.074675 | 0.230328 | 0.028748 | −0.1111 | −0.0661 | −0.2152 | 0.747368 |
| 7 | −5.16644 | 1.434343 | 0.06632 | 0.235535 | 0.018495 | −0.12242 | −0.06974 | −0.18382 | 0.663772 |
| 8 | −5.18842 | 1.350846 | 0.067308 | 0.234542 | −0.00876 | −0.1742 | −0.05766 | 0 | 0.722893 |
| 9 | −5.27103 | 1.347612 | 0.069767 | 0.25461 | 0.02144 | −0.13294 | 0 | −0.20748 | 0.766195 |
| 10 | −5.28533 | 1.329705 | 0.069699 | 0.268296 | 0.006833 | 0 | −0.05983 | −0.22182 | 0.752048 |
| 11 | −5.29835 | 1.351054 | 0.069992 | 0.26362 | 0 | −0.12526 | −0.05246 | −0.20401 | 0.760865 |
| 12 | −5.07902 | 1.367809 | 0.066019 | 0 | 0.049443 | −0.14244 | −0.03676 | −0.18908 | 0.724765 |
| 13 | −0.76315 | 1.037869 | 0 | 0.135168 | 0.027777 | −0.11632 | −0.04365 | −0.15913 | 0.872533 |
| 14 | −3.74125 | 0 | 0.058176 | 0.287584 | 0.113256 | −0.1045 | −0.06415 | −0.18012 | 0.845635 |
| 15 | −3.69839 | 0 | 0.057603 | 0.280846 | 0.098542 | −0.06592 | −0.06931 | −0.21998 | 0.849655 |
| 16 | −3.71163 | 0 | 0.057766 | 0.301222 | 0.114188 | −0.09409 | −0.07016 | −0.21309 | 0.858118 |
| 17 | −4.03192 | 0 | 0.063633 | 0.260553 | 0.094363 | −0.08496 | −0.07665 | −0.22854 | 0.89344 |
| 18 | −3.50129 | 0 | 0.053641 | 0.265595 | 0.093089 | −0.08824 | −0.08329 | −0.19554 | 0.76698 |
| 19 | −3.61201 | 0 | 0.055452 | 0.266817 | 0.060243 | −0.14249 | −0.06742 | 0 | 0.833214 |
| 20 | −3.66249 | 0 | 0.057402 | 0.278964 | 0.089578 | −0.09955 | 0 | −0.21745 | 0.874804 |
| 21 | −3.70832 | 0 | 0.057718 | 0.295718 | 0.080522 | 0 | −0.06772 | −0.22885 | 0.86013 |
| 22 | −3.68867 | 0 | 0.057567 | 0.296457 | 0 | −0.0795 | −0.05617 | −0.20838 | 0.872655 |
| 23 | −3.40905 | 0 | 0.052847 | 0 | 0.120146 | −0.11154 | −0.03721 | −0.19948 | 0.834952 |
| 24 | −0.11734 | 0 | 0 | 0.17203 | 0.087843 | −0.09899 | −0.05549 | −0.17822 | 0.957053 |
| 25 | −5.43022 | 1.391924 | 0.07117 | 0.244129 | 0.044762 | −0.1019 | −0.05779 | −0.17195 | 0.709965 |
| 26 | −5.36524 | 1.358776 | 0.070555 | 0.263277 | 0.054086 | −0.13344 | −0.05766 | −0.1771 | 0.723788 |
| 27 | −5.3502 | 1.381941 | 0.070216 | 0.260705 | 0.040541 | −0.0994 | −0.06166 | −0.2039 | 0.736272 |
| 28 | −5.61159 | 1.335815 | 0.074883 | 0.226262 | 0.041408 | −0.11586 | −0.06561 | −0.19121 | 0.71126 |
| 29 | −5.5977 | 1.347508 | 0.07483 | 0.225021 | 0.026498 | −0.08903 | −0.06837 | −0.21597 | 0.734935 |
| 30 | −5.61177 | 1.336649 | 0.074964 | 0.23534 | 0.043389 | −0.10852 | −0.07032 | −0.20998 | 0.727891 |
| 31 | −5.25408 | 1.443765 | 0.067217 | 0.229644 | 0.03237 | −0.12671 | −0.06932 | −0.15966 | 0.622205 |
| 32 | −5.21273 | 1.459722 | 0.066646 | 0.227486 | 0.017143 | −0.09922 | −0.07195 | −0.18425 | 0.644982 |
| 33 | −5.20017 | 1.442911 | 0.06651 | 0.241822 | 0.030532 | −0.12125 | −0.07338 | −0.18004 | 0.641855 |
| 34 | −5.4243 | 1.406504 | 0.070778 | 0.217664 | 0.021582 | −0.11102 | −0.07458 | −0.1941 | 0.655592 |
| 35 | −5.2358 | 1.349262 | 0.068008 | 0.234904 | 0.00842 | −0.17469 | −0.05695 | 0 | 0.715683 |
| 36 | −5.22974 | 1.377083 | 0.067597 | 0.227414 | −0.00883 | −0.14928 | −0.06076 | 0 | 0.709674 |
| 37 | −5.2021 | 1.358044 | 0.067299 | 0.240965 | 3.99E−05 | −0.17109 | −0.06041 | 0 | 0.713384 |
| 38 | −5.47665 | 1.33768 | 0.07194 | 0.202239 | −0.01401 | −0.15559 | −0.07022 | 0 | 0.689091 |
| 39 | −5.10597 | 1.440484 | 0.064496 | 0.213729 | −0.01775 | −0.16281 | −0.07015 | 0 | 0.638636 |
| 40 | −5.33027 | 1.354671 | 0.070361 | 0.249432 | 0.035864 | −0.13802 | 0 | −0.18109 | 0.741085 |
| 41 | −5.31292 | 1.37349 | 0.070076 | 0.246707 | 0.01833 | −0.10782 | 0 | −0.20873 | 0.754069 |
| 42 | −5.27991 | 1.354828 | 0.069669 | 0.261434 | 0.03336 | −0.13125 | 0 | −0.20384 | 0.756004 |
| 43 | −5.55399 | 1.329275 | 0.074594 | 0.220983 | 0.019399 | −0.11415 | 0 | −0.21698 | 0.753984 |
| 44 | −5.14134 | 1.438098 | 0.066073 | 0.224975 | 0.008532 | −0.12536 | 0 | −0.1845 | 0.669639 |
| 45 | −5.16437 | 1.353033 | 0.067074 | 0.226702 | −0.01698 | −0.17754 | 0 | 0 | 0.72939 |
| 46 | −5.34534 | 1.337341 | 0.070271 | 0.262924 | 0.019963 | 0 | −0.0591 | −0.19577 | 0.723729 |
| 47 | −5.32006 | 1.356404 | 0.069972 | 0.260376 | 0.008916 | 0 | −0.06169 | −0.22031 | 0.746164 |
| 48 | −5.29924 | 1.33599 | 0.069681 | 0.275129 | 0.018902 | 0 | −0.06298 | −0.2178 | 0.741658 |
| 49 | −5.56102 | 1.313845 | 0.074435 | 0.23714 | 0.010191 | 0 | −0.06953 | −0.22833 | 0.743168 |
| 50 | −5.16549 | 1.419913 | 0.066143 | 0.242634 | −0.00196 | 0 | −0.07337 | −0.1988 | 0.655102 |
| 51 | −5.17296 | 1.331237 | 0.066747 | 0.241758 | −0.0439 | 0 | −0.06452 | 0 | 0.705454 |
| 52 | −5.26071 | 1.331333 | 0.069455 | 0.259985 | −0.00228 | 0 | 0 | −0.22349 | 0.757889 |
| 53 | −5.36172 | 1.361051 | 0.070611 | 0.259133 | 0 | −0.12849 | −0.05088 | −0.17653 | 0.734545 |
| 54 | −5.33846 | 1.375921 | 0.070276 | 0.256127 | 0 | −0.10032 | −0.0553 | −0.2055 | 0.749088 |
| 55 | −5.31516 | 1.360525 | 0.069982 | 0.271097 | 0 | −0.12188 | −0.05522 | −0.19925 | 0.75009 |
| 56 | −5.57521 | 1.331505 | 0.074709 | 0.23245 | 0 | −0.10689 | −0.06376 | −0.21268 | 0.748227 |
| 57 | −5.16912 | 1.438053 | 0.066321 | 0.236833 | 0 | −0.11962 | −0.06822 | −0.18219 | 0.664927 |
| 58 | −5.18689 | 1.349025 | 0.067302 | 0.233953 | 0 | −0.17591 | −0.0584 | 0 | 0.722587 |
| 59 | −5.27661 | 1.351785 | 0.069797 | 0.256309 | 0 | −0.1295 | 0 | −0.20575 | 0.766559 |
| 60 | −5.28679 | 1.33119 | 0.069707 | 0.268723 | 0 | 0 | −0.05922 | −0.22112 | 0.752312 |
| 61 | −5.15212 | 1.376601 | 0.066822 | 0 | 0.062355 | −0.14697 | −0.0363 | −0.16501 | 0.699165 |
| 62 | −5.12055 | 1.390217 | 0.066375 | 0 | 0.047638 | −0.1186 | −0.03978 | −0.19061 | 0.716074 |
| 63 | −5.09083 | 1.377366 | 0.065902 | 0 | 0.060195 | −0.14091 | −0.04063 | −0.18492 | 0.709395 |
| 64 | −5.37712 | 1.347178 | 0.071147 | 0 | 0.048955 | −0.12473 | −0.04786 | −0.19955 | 0.722027 |
| 65 | −4.99096 | 1.453465 | 0.063046 | 0 | 0.037735 | −0.13528 | −0.05023 | −0.16986 | 0.63441 |
| 66 | −5.00988 | 1.37346 | 0.064043 | 0 | 0.009506 | −0.18269 | −0.04038 | 0 | 0.694844 |
| 67 | −5.06435 | 1.36751 | 0.065897 | 0 | 0.044507 | −0.14356 | 0 | −0.19002 | 0.729274 |
| 68 | −5.06971 | 1.351044 | 0.065683 | 0 | 0.026379 | 0 | −0.03975 | −0.20566 | 0.71507 |
| 69 | −5.08985 | 1.377669 | 0.066063 | 0 | 0 | −0.13522 | −0.03286 | −0.1846 | 0.725802 |
| 70 | −0.78875 | 1.045781 | 0 | 0.134406 | 0.039327 | −0.12022 | −0.04124 | −0.12973 | 0.846986 |
| 71 | −0.78442 | 1.059672 | 0 | 0.125479 | 0.029443 | −0.08628 | −0.04791 | −0.15976 | 0.859822 |
| 72 | −0.78488 | 1.049423 | 0 | 0.141556 | 0.038625 | −0.11443 | −0.04131 | −0.15499 | 0.864776 |
| 73 | −0.79625 | 1.017771 | 0 | 0.095443 | 0.026146 | −0.095 | −0.06764 | −0.16428 | 0.802539 |
| 74 | −0.82411 | 1.157554 | 0 | 0.128809 | 0.010805 | −0.11314 | −0.05952 | −0.15181 | 0.872249 |
| 75 | −0.82077 | 1.059337 | 0 | 0.120268 | −0.0096 | −0.14781 | −0.04534 | 0 | 0.847498 |
| 76 | −0.75754 | 1.041221 | 0 | 0.128332 | 0.022384 | −0.11786 | 0 | −0.15992 | 0.87675 |
| 77 | −0.77702 | 1.028722 | 0 | 0.143954 | 0.004273 | 0 | −0.04651 | −0.17179 | 0.861498 |
| 78 | −0.76837 | 1.044306 | 0 | 0.137997 | 0 | −0.11116 | −0.04143 | −0.15615 | 0.873202 |
| 79 | −0.78509 | 1.058149 | 0 | 0 | 0.043001 | −0.12624 | −0.02954 | −0.15273 | 0.852629 |
| 80 | −3.76954 | 0 | 0.05825 | 0.272781 | 0.115245 | −0.06262 | −0.06927 | −0.17185 | 0.811754 |
| 81 | −3.7637 | 0 | 0.05832 | 0.298112 | 0.125908 | −0.10128 | −0.0686 | −0.17776 | 0.835677 |
| 82 | −3.72104 | 0 | 0.057677 | 0.291212 | 0.114572 | −0.05783 | −0.07496 | −0.2169 | 0.836077 |
| 83 | −4.06118 | 0 | 0.063716 | 0.258318 | 0.10652 | −0.09056 | −0.07497 | −0.19511 | 0.86313 |
| 84 | −4.03953 | 0 | 0.063608 | 0.253812 | 0.09296 | −0.05586 | −0.08006 | −0.23108 | 0.876964 |
| 85 | −4.09161 | 0 | 0.064229 | 0.270567 | 0.111891 | −0.0804 | −0.08242 | −0.22319 | 0.876918 |
| 86 | −3.5794 | 0 | 0.054448 | 0.262108 | 0.105837 | −0.09305 | −0.082 | −0.16094 | 0.72808 |
| 87 | −3.52916 | 0 | 0.053805 | 0.255226 | 0.092432 | −0.0587 | −0.08606 | −0.19747 | 0.73526 |
| 88 | −3.54608 | 0 | 0.054047 | 0.27664 | 0.107489 | −0.08502 | −0.0887 | −0.19288 | 0.747492 |
| 89 | −3.79704 | 0 | 0.058801 | 0.248082 | 0.092473 | −0.08166 | −0.08705 | −0.2072 | 0.790597 |
| 90 | −3.66009 | 0 | 0.056184 | 0.270394 | 0.078805 | −0.14303 | −0.06615 | 0 | 0.832196 |
| 91 | −3.62064 | 0 | 0.055396 | 0.256309 | 0.060429 | −0.11204 | −0.07178 | 0 | 0.812373 |
| 92 | −3.633 | 0 | 0.055579 | 0.276436 | 0.070778 | −0.13745 | −0.07108 | 0 | 0.824064 |
| 93 | −3.95628 | 0 | 0.061242 | 0.234047 | 0.051407 | −0.12905 | −0.08061 | 0 | 0.837682 |
| 94 | −3.46327 | 0 | 0.052217 | 0.247722 | 0.054903 | −0.12836 | −0.08423 | 0 | 0.741697 |
| 95 | −3.71311 | 0 | 0.057915 | 0.276502 | 0.103156 | −0.10651 | 0 | −0.18129 | 0.852852 |
| 96 | −3.67257 | 0 | 0.057395 | 0.268312 | 0.087024 | −0.06819 | 0 | −0.22116 | 0.855913 |
| 97 | −3.67647 | 0 | 0.057406 | 0.288797 | 0.103466 | −0.09632 | 0 | −0.21391 | 0.864961 |
| 98 | −4.01147 | 0 | 0.06352 | 0.246952 | 0.082859 | −0.0868 | 0 | −0.23062 | 0.900176 |
| 99 | −3.4671 | 0 | 0.053312 | 0.249754 | 0.080632 | −0.08977 | 0 | −0.19641 | 0.77263 |
| 100 | −3.58405 | 0 | 0.055207 | 0.254583 | 0.04966 | −0.14446 | 0 | 0 | 0.840441 |
| 101 | −3.75835 | 0 | 0.058192 | 0.292334 | 0.092343 | 0 | −0.06643 | −0.19303 | 0.834853 |
| 102 | −3.70827 | 0 | 0.057633 | 0.284855 | 0.085313 | 0 | −0.07082 | −0.22877 | 0.847084 |
| 103 | −3.72896 | 0 | 0.057819 | 0.306175 | 0.095213 | 0 | −0.07229 | −0.22488 | 0.850227 |
| 104 | −4.04599 | 0 | 0.063666 | 0.265978 | 0.077679 | 0 | −0.07806 | −0.23883 | 0.886861 |
| 105 | −3.51978 | 0 | 0.053713 | 0.270288 | 0.076189 | 0 | −0.08463 | −0.20686 | 0.757574 |
| 106 | −3.62542 | 0 | 0.055266 | 0.271844 | 0.027128 | 0 | −0.07049 | 0 | 0.816499 |
| 107 | −3.68019 | 0 | 0.057459 | 0.283271 | 0.069238 | 0 | 0 | −0.23019 | 0.866479 |
| 108 | −3.73959 | 0 | 0.05809 | 0.293863 | 0 | −0.08448 | −0.05379 | −0.17109 | 0.848382 |
| 109 | −3.7012 | 0 | 0.057594 | 0.286896 | 0 | −0.04862 | −0.05994 | −0.21259 | 0.854606 |
| 110 | −3.7085 | 0 | 0.05763 | 0.307177 | 0 | −0.07381 | −0.0599 | −0.20285 | 0.860824 |
| 111 | −4.03219 | 0 | 0.063582 | 0.266988 | 0 | −0.06875 | −0.06831 | −0.22069 | 0.898996 |
| 112 | −3.49084 | 0 | 0.053442 | 0.271143 | 0 | −0.0723 | −0.07484 | −0.18701 | 0.774131 |
| 113 | −3.60786 | 0 | 0.055374 | 0.270599 | 0 | −0.12923 | −0.06161 | 0 | 0.836461 |
| 114 | −3.66671 | 0 | 0.057386 | 0.285896 | 0 | −0.08309 | 0 | −0.21004 | 0.878033 |
| 115 | −3.70385 | 0 | 0.05763 | 0.299916 | 0 | 0 | −0.05963 | −0.22002 | 0.865309 |
| 116 | −3.46666 | 0 | 0.05346 | 0 | 0.13232 | −0.11684 | −0.03658 | −0.16614 | 0.811598 |
| 117 | −3.42745 | 0 | 0.053006 | 0 | 0.118838 | −0.08179 | −0.04121 | −0.20283 | 0.820986 |
| 118 | −3.42078 | 0 | 0.052761 | 0 | 0.132576 | −0.10854 | −0.04166 | −0.19516 | 0.819177 |
| 119 | −3.77486 | 0 | 0.059278 | 0 | 0.115455 | −0.10051 | −0.04976 | −0.21242 | 0.869826 |
| 120 | −3.26475 | 0 | 0.049564 | 0 | 0.111682 | −0.10093 | −0.05395 | −0.18281 | 0.738736 |
| 121 | −3.36687 | 0 | 0.051338 | 0 | 0.079131 | −0.15061 | −0.04051 | 0 | 0.806238 |
| 122 | −3.39946 | 0 | 0.052808 | 0 | 0.114193 | −0.11167 | 0 | −0.20039 | 0.839304 |
| 123 | −3.42799 | 0 | 0.052876 | 0 | 0.098464 | 0 | −0.0376 | −0.21261 | 0.824836 |
| 124 | −3.41317 | 0 | 0.05282 | 0 | 0 | −0.09054 | −0.02593 | −0.18839 | 0.839603 |
| 125 | −0.13658 | 0 | 0 | 0.171119 | 0.099299 | −0.10294 | −0.0532 | −0.14169 | 0.935227 |
| 126 | −0.12794 | 0 | 0 | 0.162284 | 0.090626 | −0.06458 | −0.05998 | −0.18042 | 0.941853 |
| 127 | −0.13407 | 0 | 0 | 0.17986 | 0.099515 | −0.09469 | −0.05422 | −0.17437 | 0.949573 |
| 128 | −0.13815 | 0 | 0 | 0.130671 | 0.081616 | −0.08267 | −0.07772 | −0.18402 | 0.929566 |
| 129 | −0.1303 | 0 | 0 | 0.167927 | 0.075508 | −0.09063 | −0.07265 | −0.17066 | 0.935785 |
| 130 | −0.16671 | 0 | 0 | 0.158422 | 0.048713 | −0.13002 | −0.05792 | 0 | 0.930146 |
| 131 | −0.10717 | 0 | 0 | 0.163369 | 0.08093 | −0.10021 | 0 | −0.1793 | 0.961962 |
| 132 | −0.13345 | 0 | 0 | 0.178718 | 0.066245 | 0 | −0.05703 | −0.18838 | 0.945823 |
| 133 | −0.12112 | 0 | 0 | 0.18122 | 0 | −0.0812 | −0.04829 | −0.16923 | 0.960497 |
| 134 | −0.12827 | 0 | 0 | 0 | 0.107366 | −0.1102 | −0.03736 | −0.17119 | 0.934124 |
| 135 | −5.48716 | 1.417484 | 0.071289 | 0.22219 | 0.035461 | −0.10876 | −0.07884 | −0.18943 | 0.62239 |
| 136 | −5.44935 | 1.403292 | 0.071155 | 0.250132 | 0.057909 | −0.09695 | −0.06177 | −0.16816 | 0.695566 |
| 137 | −5.6696 | 1.370803 | 0.075171 | 0.217364 | 0.042461 | −0.08738 | −0.06834 | −0.18317 | 0.680439 |
| 138 | −5.65339 | 1.345786 | 0.075163 | 0.231028 | 0.054284 | −0.11286 | −0.06967 | −0.18714 | 0.690164 |
| 139 | −5.64511 | 1.362437 | 0.075113 | 0.229515 | 0.042738 | −0.08241 | −0.0733 | −0.2107 | 0.712898 |
| 140 | −5.35503 | 1.482373 | 0.068039 | 0.216953 | 0.034492 | −0.09705 | −0.07231 | −0.15274 | 0.577481 |
| 141 | −5.29022 | 1.452416 | 0.06743 | 0.235287 | 0.04312 | −0.125 | −0.07288 | −0.15644 | 0.596788 |
| 142 | −5.25529 | 1.472659 | 0.06688 | 0.232879 | 0.030668 | −0.09535 | −0.07599 | −0.18026 | 0.617452 |
| 143 | −5.48835 | 1.418871 | 0.071192 | 0.213391 | 0.033881 | −0.1151 | −0.07418 | −0.17152 | 0.606258 |
| 144 | −5.45817 | 1.429935 | 0.070924 | 0.211726 | 0.020035 | −0.09033 | −0.07665 | −0.19398 | 0.635879 |
| 145 | −5.31149 | 1.387246 | 0.068665 | 0.224922 | 0.013463 | −0.13971 | −0.06081 | 0 | 0.696808 |
| 146 | −5.24778 | 1.356211 | 0.067981 | 0.241547 | 0.016866 | −0.17175 | −0.0597 | 0 | 0.706008 |
| 147 | −5.24863 | 1.388756 | 0.067602 | 0.233174 | 0.001409 | −0.14277 | −0.06392 | 0 | 0.698225 |
| 148 | −5.50857 | 1.33799 | 0.072318 | 0.203358 | 0.00131 | −0.1555 | −0.06943 | 0 | 0.675926 |
| 149 | −5.50851 | 1.359714 | 0.072109 | 0.19729 | −0.01398 | −0.13464 | −0.07266 | 0 | 0.673478 |
| 150 | −5.51846 | 1.348774 | 0.072234 | 0.207429 | −0.00331 | −0.15062 | −0.0738 | 0 | 0.670614 |
| 151 | −5.16679 | 1.441927 | 0.065283 | 0.212866 | −0.00102 | −0.16239 | −0.06975 | 0 | 0.618637 |
| 152 | −5.15584 | 1.466058 | 0.064871 | 0.205999 | −0.01713 | −0.13974 | −0.07276 | 0 | 0.618299 |
| 153 | −5.13819 | 1.449924 | 0.064672 | 0.219748 | −0.00823 | −0.16005 | −0.07328 | 0 | 0.617823 |
| 154 | −5.36954 | 1.415374 | 0.068848 | 0.194411 | −0.01806 | −0.15161 | −0.07642 | 0 | 0.613653 |
| 155 | −5.40996 | 1.394649 | 0.070993 | 0.236481 | 0.036205 | −0.10488 | 0 | −0.17317 | 0.716515 |
| 156 | −5.33765 | 1.361207 | 0.070256 | 0.256166 | 0.046646 | −0.13622 | 0 | −0.17813 | 0.730583 |
| 157 | −5.32617 | 1.385335 | 0.069977 | 0.253012 | 0.032044 | −0.10274 | 0 | −0.20502 | 0.742082 |
| 158 | −5.59464 | 1.338619 | 0.074769 | 0.216989 | 0.032262 | −0.11864 | 0 | −0.19291 | 0.718489 |
| 159 | −5.58496 | 1.351366 | 0.074778 | 0.215235 | 0.016319 | −0.09232 | 0 | −0.21779 | 0.740992 |
| 160 | −5.58997 | 1.340438 | 0.074773 | 0.226029 | 0.034561 | −0.11113 | 0 | −0.21171 | 0.735145 |
| 161 | −5.22569 | 1.447119 | 0.066928 | 0.219202 | 0.022546 | −0.12936 | 0 | −0.16028 | 0.629214 |
| 162 | −5.19129 | 1.463828 | 0.06646 | 0.216598 | 0.006467 | −0.10228 | 0 | −0.18501 | 0.65039 |
| 163 | −5.16905 | 1.447179 | 0.066167 | 0.231112 | 0.020775 | −0.12399 | 0 | −0.18056 | 0.647732 |
| 164 | −5.40496 | 1.410546 | 0.070631 | 0.206295 | 0.010965 | −0.11384 | 0 | −0.19532 | 0.661649 |
| 165 | −5.21062 | 1.351262 | 0.067754 | 0.227226 | 0.000543 | −0.17775 | 0 | 0 | 0.722571 |
| 166 | −5.20744 | 1.379737 | 0.067397 | 0.219084 | −0.01787 | −0.15285 | 0 | 0 | 0.71577 |
| 167 | −5.17312 | 1.360462 | 0.066989 | 0.233164 | −0.00797 | −0.17428 | 0 | 0 | 0.720019 |
| 168 | −5.45753 | 1.340958 | 0.071801 | 0.192082 | −0.02394 | −0.15891 | 0 | 0 | 0.695979 |
| 169 | −5.07813 | 1.444228 | 0.064217 | 0.20304 | −0.02765 | −0.16579 | 0 | 0 | 0.645428 |
| 170 | −5.41561 | 1.378121 | 0.070861 | 0.249924 | 0.027108 | 0 | −0.06133 | −0.18408 | 0.707755 |
| 171 | −5.35815 | 1.343044 | 0.070251 | 0.269557 | 0.030846 | 0 | −0.06205 | −0.19244 | 0.71285 |
| 172 | −5.33718 | 1.367479 | 0.069945 | 0.266666 | 0.02352 | 0 | −0.06535 | −0.21592 | 0.735046 |
| 173 | −5.60261 | 1.3237 | 0.074595 | 0.233061 | 0.022134 | 0 | −0.06886 | −0.20436 | 0.705027 |
| 174 | −5.58714 | 1.336007 | 0.074618 | 0.230899 | 0.011578 | 0 | −0.07126 | −0.22693 | 0.73584 |
| 175 | −5.60315 | 1.324038 | 0.074705 | 0.241987 | 0.025275 | 0 | −0.07309 | −0.22237 | 0.723249 |
| 176 | −5.25404 | 1.42924 | 0.067023 | 0.23659 | 0.011165 | 0 | −0.07271 | −0.17482 | 0.609899 |
| 177 | −5.2024 | 1.446455 | 0.066411 | 0.2339 | 0.000395 | 0 | −0.07495 | −0.19666 | 0.643502 |
| 178 | −5.20017 | 1.427839 | 0.066345 | 0.248662 | 0.010231 | 0 | −0.07666 | −0.19457 | 0.632178 |
| 179 | −5.41874 | 1.394129 | 0.070556 | 0.224707 | 0.003178 | 0 | −0.07747 | −0.20731 | 0.649704 |
| 180 | −5.21628 | 1.329591 | 0.067365 | 0.24161 | −0.02736 | 0 | −0.0634 | 0 | 0.696805 |
| 181 | −5.20673 | 1.357942 | 0.067039 | 0.234587 | −0.03878 | 0 | −0.06671 | 0 | 0.701374 |
| 182 | −5.186 | 1.33765 | 0.06674 | 0.248104 | −0.03483 | 0 | −0.06668 | 0 | 0.696428 |
| 183 | −5.45783 | 1.321342 | 0.071356 | 0.210147 | −0.04501 | 0 | −0.07526 | 0 | 0.674805 |
| 184 | −5.1008 | 1.421741 | 0.064103 | 0.221342 | −0.05005 | 0 | −0.07517 | 0 | 0.620585 |
| 185 | −5.31933 | 1.338746 | 0.070006 | 0.254723 | 0.011172 | 0 | 0 | −0.19729 | 0.730141 |
| 186 | −5.29828 | 1.358473 | 0.069775 | 0.251867 | −0.00096 | 0 | 0 | −0.22208 | 0.751727 |
| 187 | −5.26898 | 1.338047 | 0.069348 | 0.266776 | 0.010001 | 0 | 0 | −0.2193 | 0.747558 |
| 188 | −5.54383 | 1.316455 | 0.074319 | 0.226898 | −0.00019 | 0 | 0 | −0.23053 | 0.749448 |
| 189 | −5.13755 | 1.422851 | 0.065858 | 0.230989 | −0.01296 | 0 | 0 | −0.19987 | 0.660449 |
| 190 | −5.14331 | 1.332268 | 0.06644 | 0.232118 | −0.05385 | 0 | 0 | 0 | 0.711494 |
| 191 | −5.44113 | 1.4004 | 0.071229 | 0.247039 | 0 | −0.0952 | −0.0541 | −0.16866 | 0.70996 |
| 192 | −5.37658 | 1.369651 | 0.070586 | 0.266332 | 0 | −0.12521 | −0.05361 | −0.17246 | 0.722875 |
| 193 | −5.3606 | 1.390193 | 0.070275 | 0.263262 | 0 | −0.09335 | −0.05848 | −0.20062 | 0.736523 |
| 194 | −5.62067 | 1.343714 | 0.074918 | 0.22917 | 0 | −0.10987 | −0.06229 | −0.18753 | 0.711344 |
| 195 | −5.6046 | 1.352585 | 0.074876 | 0.227044 | 0 | −0.08517 | −0.06613 | −0.21379 | 0.735872 |
| 196 | −5.62148 | 1.345418 | 0.074998 | 0.238401 | 0 | −0.10225 | −0.06712 | −0.20568 | 0.727947 |
| 197 | −5.25922 | 1.450098 | 0.067223 | 0.231798 | 0 | −0.12188 | −0.06668 | −0.15673 | 0.623205 |
| 198 | −5.21564 | 1.463102 | 0.066656 | 0.228754 | 0 | −0.09664 | −0.0705 | −0.18284 | 0.646151 |
| 199 | −5.205 | 1.449249 | 0.06651 | 0.243804 | 0 | −0.11666 | −0.07099 | −0.17707 | 0.642911 |
| 200 | −5.42823 | 1.410737 | 0.070792 | 0.219277 | 0 | −0.10785 | −0.07279 | −0.1921 | 0.656872 |
| 201 | −5.23746 | 1.351018 | 0.068015 | 0.235436 | 0 | −0.17308 | −0.05626 | 0 | 0.715718 |
| 202 | −5.22799 | 1.375294 | 0.067588 | 0.226797 | 0 | −0.15095 | −0.06153 | 0 | 0.70937 |
| 203 | −5.20211 | 1.358053 | 0.067299 | 0.240968 | 0 | −0.17108 | −0.0604 | 0 | 0.713385 |
| 204 | −5.47394 | 1.334825 | 0.071925 | 0.201135 | 0 | −0.15824 | −0.07141 | 0 | 0.688324 |
| 205 | −5.10424 | 1.436804 | 0.064501 | 0.21239 | 0 | −0.16622 | −0.07166 | 0 | 0.637324 |
| 206 | −5.33912 | 1.361486 | 0.070401 | 0.252131 | 0 | −0.13233 | 0 | −0.17815 | 0.740795 |
| 207 | −5.31821 | 1.376994 | 0.070111 | 0.24824 | 0 | −0.10491 | 0 | −0.20734 | 0.754479 |
| 208 | −5.28863 | 1.361515 | 0.069712 | 0.263869 | 0 | −0.12596 | 0 | −0.20089 | 0.755921 |
| 209 | −5.55897 | 1.332905 | 0.07462 | 0.222717 | 0 | −0.11122 | 0 | −0.21523 | 0.754403 |
| 210 | −5.14293 | 1.43975 | 0.066078 | 0.225722 | 0 | −0.12403 | 0 | −0.18373 | 0.670109 |
| 211 | −5.16065 | 1.349603 | 0.067055 | 0.225291 | 0 | −0.18095 | 0 | 0 | 0.72896 |
| 212 | −5.34938 | 1.341552 | 0.070287 | 0.264121 | 0 | 0 | −0.05736 | −0.19368 | 0.724052 |
| 213 | −5.3224 | 1.358352 | 0.069988 | 0.260949 | 0 | 0 | −0.06087 | −0.21944 | 0.746482 |
| 214 | −5.30318 | 1.340239 | 0.069697 | 0.27621 | 0 | 0 | −0.06139 | −0.21569 | 0.742007 |
| 215 | −5.56331 | 1.315956 | 0.074448 | 0.237843 | 0 | 0 | −0.06863 | −0.22719 | 0.743534 |
| 216 | −5.16525 | 1.419485 | 0.066144 | 0.242505 | 0 | 0 | −0.07354 | −0.19902 | 0.654952 |
| 217 | −5.16618 | 1.32117 | 0.066716 | 0.238805 | 0 | 0 | −0.06879 | 0 | 0.702778 |
| 218 | −5.26012 | 1.330848 | 0.069451 | 0.259802 | 0 | 0 | 0 | −0.22373 | 0.75782 |
| 219 | −5.23735 | 1.412138 | 0.067608 | 0 | 0.063965 | −0.1153 | −0.04026 | −0.15833 | 0.678951 |
| 220 | −5.16328 | 1.38558 | 0.066706 | 0 | 0.072081 | −0.14523 | −0.04003 | −0.16172 | 0.683089 |
| 221 | −5.13803 | 1.404085 | 0.06628 | 0 | 0.060123 | −0.11405 | −0.04442 | −0.18628 | 0.698999 |
| 222 | −5.42899 | 1.35809 | 0.071493 | 0 | 0.060474 | −0.12872 | −0.04751 | −0.17743 | 0.686486 |
| 223 | −5.40611 | 1.366627 | 0.071338 | 0 | 0.046954 | −0.10336 | −0.05035 | −0.20059 | 0.711907 |
| 224 | −5.41815 | 1.361165 | 0.071336 | 0 | 0.062899 | −0.12216 | −0.05299 | −0.19393 | 0.697877 |
| 225 | −5.08988 | 1.463962 | 0.06412 | 0 | 0.050389 | −0.13871 | −0.05016 | −0.14784 | 0.593213 |
| 226 | −5.03796 | 1.476095 | 0.063441 | 0 | 0.036772 | −0.113 | −0.05294 | −0.17064 | 0.619711 |
| 227 | −5.02867 | 1.465124 | 0.063212 | 0 | 0.048745 | −0.13385 | −0.05413 | −0.16556 | 0.605251 |
| 228 | −5.25109 | 1.42482 | 0.067607 | 0 | 0.040642 | −0.12418 | −0.05584 | −0.18061 | 0.634437 |
| 229 | −5.06553 | 1.374373 | 0.064833 | 0 | 0.025977 | −0.18265 | −0.03958 | 0 | 0.685811 |
| 230 | −5.0525 | 1.396359 | 0.064396 | 0 | 0.009552 | −0.1591 | −0.04391 | 0 | 0.683965 |
| 231 | −5.02192 | 1.38278 | 0.063957 | 0 | 0.017927 | −0.17979 | −0.04348 | 0 | 0.680947 |
| 232 | −5.31782 | 1.357367 | 0.06908 | 0 | 0.004156 | −0.1649 | −0.05377 | 0 | 0.672755 |
| 233 | −4.9555 | 1.460155 | 0.061714 | 0 | 0.000181 | −0.17161 | −0.05226 | 0 | 0.6146 |
| 234 | −5.1367 | 1.376235 | 0.066688 | 0 | 0.057585 | −0.14798 | 0 | −0.16587 | 0.704042 |
| 235 | −5.10647 | 1.390134 | 0.066269 | 0 | 0.041982 | −0.11987 | 0 | −0.19168 | 0.720522 |
| 236 | −5.07253 | 1.37716 | 0.06573 | 0 | 0.055145 | −0.142 | 0 | −0.1858 | 0.714082 |
| 237 | −5.36605 | 1.347289 | 0.071108 | 0 | 0.042344 | −0.12581 | 0 | −0.20121 | 0.727763 |
| 238 | −4.9728 | 1.45374 | 0.062898 | 0 | 0.030763 | −0.13616 | 0 | −0.17072 | 0.639882 |
| 239 | −4.99293 | 1.373068 | 0.063899 | 0 | 0.003934 | −0.18406 | 0 | 0 | 0.699983 |
| 240 | −5.14243 | 1.359916 | 0.066448 | 0 | 0.038557 | 0 | −0.03909 | −0.18172 | 0.686641 |
| 241 | −5.10409 | 1.374344 | 0.066013 | 0 | 0.028438 | 0 | −0.0424 | −0.20469 | 0.712846 |
| 242 | −5.08165 | 1.360082 | 0.065569 | 0 | 0.037298 | 0 | −0.04329 | −0.20104 | 0.699446 |
| 243 | −5.36437 | 1.334111 | 0.070789 | 0 | 0.029336 | 0 | −0.04972 | −0.21363 | 0.716046 |
| 244 | −4.98494 | 1.43721 | 0.062757 | 0 | 0.016471 | 0 | −0.05197 | −0.18574 | 0.623303 |
| 245 | −4.99044 | 1.352018 | 0.063394 | 0 | −0.02592 | 0 | −0.0447 | 0 | 0.675605 |
| 246 | −5.05301 | 1.350054 | 0.065539 | 0 | 0.020819 | 0 | 0 | −0.20681 | 0.719509 |
| 247 | −5.1657 | 1.388736 | 0.066871 | 0 | 0 | −0.138 | −0.03149 | −0.1593 | 0.699011 |
| 248 | −5.13177 | 1.399566 | 0.066433 | 0 | 0 | −0.11177 | −0.03586 | −0.18656 | 0.717243 |
| 249 | −5.10415 | 1.389638 | 0.065948 | 0 | 0 | −0.13219 | −0.03617 | −0.17897 | 0.709484 |
| 250 | −5.38718 | 1.356737 | 0.071178 | 0 | 0 | −0.11793 | −0.04389 | −0.1947 | 0.723099 |
| 251 | −4.99584 | 1.460983 | 0.063036 | 0 | 0 | −0.1299 | −0.04714 | −0.1661 | 0.636582 |
| 252 | −5.01161 | 1.37545 | 0.064049 | 0 | 0 | −0.1809 | −0.03957 | 0 | 0.695161 |
| 253 | −5.07556 | 1.376384 | 0.065948 | 0 | 0 | −0.1369 | 0 | −0.18583 | 0.729746 |
| 254 | −5.07494 | 1.356615 | 0.065704 | 0 | 0 | 0 | −0.03752 | −0.20261 | 0.715944 |
| 255 | −0.82904 | 1.079898 | 0 | 0.120852 | 0.044853 | −0.07915 | −0.04605 | −0.12013 | 0.81905 |
| 256 | −0.80871 | 1.057306 | 0 | 0.140944 | 0.049362 | −0.11882 | −0.03879 | −0.12596 | 0.841067 |
| 257 | −0.81111 | 1.07517 | 0 | 0.131571 | 0.0426 | −0.07977 | −0.04663 | −0.15551 | 0.849437 |
| 258 | −0.82793 | 1.028944 | 0 | 0.095062 | 0.036339 | −0.09702 | −0.0646 | −0.14037 | 0.766641 |
| 259 | −0.8152 | 1.038104 | 0 | 0.087777 | 0.027056 | −0.06996 | −0.06954 | −0.16464 | 0.789868 |
| 260 | −0.83816 | 1.037774 | 0 | 0.097962 | 0.03897 | −0.09039 | −0.06539 | −0.15786 | 0.771879 |
| 261 | −0.8561 | 1.166035 | 0 | 0.126682 | 0.024174 | −0.11727 | −0.05606 | −0.12032 | 0.837766 |
| 262 | −0.84458 | 1.18285 | 0 | 0.118442 | 0.012783 | −0.08282 | −0.06414 | −0.15235 | 0.862793 |
| 263 | −0.85166 | 1.171982 | 0 | 0.134649 | 0.023467 | −0.11273 | −0.05716 | −0.14672 | 0.859135 |
| 264 | −0.83779 | 1.10931 | 0 | 0.098403 | 0.015981 | −0.09759 | −0.07352 | −0.15783 | 0.809934 |
| 265 | −0.82633 | 1.057708 | 0 | 0.123845 | 0.007982 | −0.14633 | −0.04275 | 0 | 0.838484 |
| 266 | −0.84221 | 1.081429 | 0 | 0.110803 | −0.00759 | −0.11818 | −0.04952 | 0 | 0.83485 |
| 267 | −0.84103 | 1.071043 | 0 | 0.126617 | 0.000486 | −0.14461 | −0.04318 | 0 | 0.840651 |
| 268 | −0.86279 | 1.041646 | 0 | 0.080133 | −0.01387 | −0.12301 | −0.06853 | 0 | 0.765211 |
| 269 | −0.87738 | 1.175335 | 0 | 0.11536 | −0.02357 | −0.14381 | −0.05913 | 0 | 0.849323 |
| 270 | −0.78323 | 1.048876 | 0 | 0.128052 | 0.034298 | −0.1217 | 0 | −0.13043 | 0.85145 |
| 271 | −0.77824 | 1.063389 | 0 | 0.117677 | 0.023186 | −0.08802 | 0 | −0.16056 | 0.863973 |
| 272 | −0.7793 | 1.05274 | 0 | 0.13551 | 0.033978 | −0.11587 | 0 | −0.15573 | 0.869301 |
| 273 | −0.79 | 1.022301 | 0 | 0.084294 | 0.018041 | −0.09621 | 0 | −0.16526 | 0.805283 |
| 274 | −0.81735 | 1.162133 | 0 | 0.118878 | 0.003825 | −0.11457 | 0 | −0.15218 | 0.875895 |
| 275 | −0.81493 | 1.063002 | 0 | 0.112965 | −0.01536 | −0.1495 | 0 | 0 | 0.851915 |
| 276 | −0.80422 | 1.036408 | 0 | 0.143234 | 0.015344 | 0 | −0.04424 | −0.14248 | 0.833605 |
| 277 | −0.79175 | 1.051498 | 0 | 0.13257 | 0.01198 | 0 | −0.05008 | −0.16948 | 0.855461 |
| 278 | −0.79792 | 1.039375 | 0 | 0.150312 | 0.015387 | 0 | −0.04409 | −0.16714 | 0.853814 |
| 279 | −0.80823 | 1.011978 | 0 | 0.103107 | 0.007021 | 0 | −0.06877 | −0.17325 | 0.793405 |
| 280 | −0.83753 | 1.147413 | 0 | 0.137032 | −0.01107 | 0 | −0.06148 | −0.16448 | 0.861196 |
| 281 | −0.84321 | 1.047481 | 0 | 0.129895 | −0.04368 | 0 | −0.04916 | 0 | 0.828695 |
| 282 | −0.77103 | 1.031919 | 0 | 0.136483 | −0.00184 | 0 | 0 | −0.17279 | 0.865624 |
| 283 | −0.79622 | 1.054693 | 0 | 0.138444 | 0 | −0.11306 | −0.03812 | −0.12556 | 0.847263 |
| 284 | −0.78997 | 1.066421 | 0 | 0.128391 | 0 | −0.08088 | −0.04547 | −0.15672 | 0.860446 |
| 285 | −0.79248 | 1.058475 | 0 | 0.145442 | 0 | −0.10729 | −0.03851 | −0.15059 | 0.865029 |
| 286 | −0.80086 | 1.023579 | 0 | 0.098251 | 0 | −0.09023 | −0.06551 | −0.16137 | 0.803553 |
| 287 | −0.82586 | 1.159982 | 0 | 0.129943 | 0 | −0.1112 | −0.0587 | −0.15062 | 0.872955 |
| 288 | −0.81927 | 1.057108 | 0 | 0.119193 | 0 | −0.14993 | −0.04614 | 0 | 0.84708 |
| 289 | −0.76199 | 1.046282 | 0 | 0.130906 | 0 | −0.11362 | 0 | −0.15747 | 0.877141 |
| 290 | −0.77773 | 1.029795 | 0 | 0.144353 | 0 | 0 | −0.04614 | −0.17121 | 0.861694 |
| 291 | −0.81112 | 1.065791 | 0 | 0 | 0.054595 | −0.12972 | −0.02727 | −0.12442 | 0.826138 |
| 292 | −0.80355 | 1.077974 | 0 | 0 | 0.043354 | −0.09632 | −0.03431 | −0.15374 | 0.84311 |
| 293 | −0.8088 | 1.071155 | 0 | 0 | 0.054117 | −0.12476 | −0.02803 | −0.14826 | 0.841954 |
| 294 | −0.81023 | 1.03207 | 0 | 0 | 0.037618 | −0.10255 | −0.05614 | −0.15974 | 0.791315 |
| 295 | −0.84583 | 1.174893 | 0 | 0 | 0.02556 | −0.12232 | −0.04516 | −0.14599 | 0.851412 |
| 296 | −0.83793 | 1.077488 | 0 | 0 | 0.00478 | −0.15539 | −0.03257 | 0 | 0.831254 |
| 297 | −0.78042 | 1.05965 | 0 | 0 | 0.038855 | −0.12677 | 0 | −0.15347 | 0.856228 |
| 298 | −0.80105 | 1.049057 | 0 | 0 | 0.018737 | 0 | −0.03099 | −0.1659 | 0.839253 |
| 299 | −0.79387 | 1.068817 | 0 | 0 | 0 | −0.11856 | −0.02567 | −0.14773 | 0.853111 |
| 300 | −3.87765 | 0 | 0.059671 | 0.258271 | 0.109218 | −0.07704 | −0.0934 | −0.20336 | 0.763097 |
| 301 | −3.79557 | 0 | 0.058366 | 0.282895 | 0.130246 | −0.05465 | −0.07474 | −0.1685 | 0.795674 |
| 302 | −4.08129 | 0 | 0.063665 | 0.248179 | 0.108518 | −0.05173 | −0.07904 | −0.18561 | 0.828476 |
| 303 | −4.12204 | 0 | 0.064351 | 0.268182 | 0.121769 | −0.08559 | −0.0805 | −0.19149 | 0.846625 |
| 304 | −4.09927 | 0 | 0.064154 | 0.263695 | 0.112215 | −0.04535 | −0.08685 | −0.22588 | 0.858028 |
| 305 | −3.65753 | 0 | 0.055065 | 0.247152 | 0.108497 | −0.05387 | −0.0858 | −0.15345 | 0.661346 |
| 306 | −3.62747 | 0 | 0.054894 | 0.272491 | 0.118715 | −0.08951 | −0.08713 | −0.15888 | 0.705581 |
| 307 | −3.58471 | 0 | 0.054277 | 0.265387 | 0.108149 | −0.05121 | −0.09228 | −0.19472 | 0.705284 |
| 308 | −3.84487 | 0 | 0.059057 | 0.245886 | 0.104224 | −0.08573 | −0.08554 | −0.17477 | 0.745859 |
| 309 | −3.81044 | 0 | 0.058757 | 0.240487 | 0.09162 | −0.0537 | −0.08988 | −0.20819 | 0.761812 |
| 310 | −3.68658 | 0 | 0.056313 | 0.25688 | 0.0846 | −0.09976 | −0.07178 | 0 | 0.803861 |
| 311 | −3.68026 | 0 | 0.056305 | 0.280293 | 0.088783 | −0.13843 | −0.06976 | 0 | 0.823018 |
| 312 | −3.64407 | 0 | 0.055506 | 0.265315 | 0.072451 | −0.10237 | −0.07616 | 0 | 0.799207 |
| 313 | −3.98549 | 0 | 0.061631 | 0.238303 | 0.068289 | −0.12852 | −0.07906 | 0 | 0.833668 |
| 314 | −3.96275 | 0 | 0.061165 | 0.226803 | 0.051734 | −0.10156 | −0.08421 | 0 | 0.817776 |
| 315 | −4.01246 | 0 | 0.06183 | 0.243439 | 0.064129 | −0.12176 | −0.08516 | 0 | 0.823235 |
| 316 | −3.51895 | 0 | 0.052968 | 0.250273 | 0.072881 | −0.1272 | −0.08315 | 0 | 0.729496 |
| 317 | −3.49405 | 0 | 0.052408 | 0.236868 | 0.055596 | −0.09919 | −0.08752 | 0 | 0.707676 |
| 318 | −3.50628 | 0 | 0.052606 | 0.257691 | 0.066399 | −0.12346 | −0.08868 | 0 | 0.722281 |
| 319 | −3.76146 | 0 | 0.057231 | 0.227649 | 0.051754 | −0.12122 | −0.08954 | 0 | 0.749159 |
| 320 | −3.74193 | 0 | 0.058017 | 0.260562 | 0.103849 | −0.0646 | 0 | −0.1732 | 0.818527 |
| 321 | −3.72808 | 0 | 0.057948 | 0.286259 | 0.115592 | −0.10332 | 0 | −0.17871 | 0.843056 |
| 322 | −3.68641 | 0 | 0.057341 | 0.277562 | 0.102647 | −0.06023 | 0 | −0.21772 | 0.842263 |
| 323 | −4.03887 | 0 | 0.063571 | 0.245311 | 0.095369 | −0.09226 | 0 | −0.19728 | 0.870514 |
| 324 | −4.01999 | 0 | 0.063516 | 0.239483 | 0.080455 | −0.05768 | 0 | −0.23316 | 0.882762 |
| 325 | −4.06015 | 0 | 0.063957 | 0.25559 | 0.10046 | −0.08213 | 0 | −0.22494 | 0.884235 |
| 326 | −3.54217 | 0 | 0.054075 | 0.246837 | 0.093635 | −0.0945 | 0 | −0.16198 | 0.734851 |
| 327 | −3.4975 | 0 | 0.053526 | 0.238924 | 0.079202 | −0.06003 | 0 | −0.19845 | 0.740389 |
| 328 | −3.50427 | 0 | 0.053602 | 0.259298 | 0.094615 | −0.08645 | 0 | −0.19332 | 0.752603 |
| 329 | −3.7686 | 0 | 0.058577 | 0.232038 | 0.079841 | −0.08303 | 0 | −0.20885 | 0.7964 |
| 330 | −3.631 | 0 | 0.055916 | 0.258772 | 0.068583 | −0.14493 | 0 | 0 | 0.839717 |
| 331 | −3.59326 | 0 | 0.055172 | 0.243064 | 0.048814 | −0.1141 | 0 | 0 | 0.81903 |
| 332 | −3.59862 | 0 | 0.055239 | 0.263657 | 0.060214 | −0.13946 | 0 | 0 | 0.831415 |
| 333 | −3.93303 | 0 | 0.061099 | 0.219349 | 0.03942 | −0.1307 | 0 | 0 | 0.844955 |
| 334 | −3.42846 | 0 | 0.051889 | 0.231671 | 0.042461 | −0.12959 | 0 | 0 | 0.748342 |
| 335 | −3.77536 | 0 | 0.05822 | 0.276164 | 0.102755 | 0 | −0.07052 | −0.17985 | 0.808794 |
| 336 | −3.7799 | 0 | 0.058321 | 0.302618 | 0.105481 | 0 | −0.07072 | −0.19002 | 0.824851 |
| 337 | −3.72852 | 0 | 0.057685 | 0.294873 | 0.103004 | 0 | −0.07627 | −0.22446 | 0.83432 |
| 338 | −4.07322 | 0 | 0.063692 | 0.263527 | 0.088924 | 0 | −0.07636 | −0.20551 | 0.854433 |
| 339 | −4.04701 | 0 | 0.063624 | 0.257647 | 0.081903 | 0 | −0.08096 | −0.23822 | 0.875316 |
| 340 | −4.10362 | 0 | 0.064225 | 0.275697 | 0.095998 | 0 | −0.0836 | −0.2325 | 0.869924 |
| 341 | −3.59806 | 0 | 0.054492 | 0.266392 | 0.088071 | 0 | −0.08333 | −0.17259 | 0.715301 |
| 342 | −3.53645 | 0 | 0.053802 | 0.258758 | 0.081085 | 0 | −0.08684 | −0.20523 | 0.73232 |
| 343 | −3.56409 | 0 | 0.054108 | 0.280984 | 0.091063 | 0 | −0.08986 | −0.20351 | 0.737495 |
| 344 | −3.81141 | 0 | 0.058826 | 0.253094 | 0.076825 | 0 | −0.08806 | −0.21707 | 0.782067 |
| 345 | −3.66915 | 0 | 0.055913 | 0.274544 | 0.045314 | 0 | −0.06914 | 0 | 0.814133 |
| 346 | −3.62581 | 0 | 0.055208 | 0.261041 | 0.034471 | 0 | −0.07413 | 0 | 0.804108 |
| 347 | −3.64524 | 0 | 0.055383 | 0.281424 | 0.038561 | 0 | −0.07396 | 0 | 0.80802 |
| 348 | −3.96281 | 0 | 0.060967 | 0.239538 | 0.021619 | 0 | −0.08256 | 0 | 0.821464 |
| 349 | −3.48062 | 0 | 0.052098 | 0.252109 | 0.025983 | 0 | −0.08599 | 0 | 0.723101 |
| 350 | −3.72921 | 0 | 0.057913 | 0.280434 | 0.081501 | 0 | 0 | −0.1944 | 0.841671 |
| 351 | −3.68194 | 0 | 0.057415 | 0.271807 | 0.073079 | 0 | 0 | −0.23023 | 0.853047 |
| 352 | −3.69274 | 0 | 0.05744 | 0.292933 | 0.083682 | 0 | 0 | −0.22593 | 0.856647 |
| 353 | −4.02498 | 0 | 0.063541 | 0.251813 | 0.065697 | 0 | 0 | −0.24107 | 0.893202 |
| 354 | −3.48528 | 0 | 0.053375 | 0.253793 | 0.063292 | 0 | 0 | −0.20787 | 0.762688 |
| 355 | −3.59536 | 0 | 0.054987 | 0.258324 | 0.015488 | 0 | 0 | 0 | 0.823263 |
| 356 | −3.77301 | 0 | 0.058216 | 0.279467 | 0 | −0.04283 | −0.05843 | −0.16327 | 0.814372 |
| 357 | −3.7601 | 0 | 0.058147 | 0.304303 | 0 | −0.07909 | −0.05745 | −0.16672 | 0.836144 |
| 358 | −3.72293 | 0 | 0.057627 | 0.297512 | 0 | −0.03807 | −0.06437 | −0.20713 | 0.838808 |
| 359 | −4.06076 | 0 | 0.06363 | 0.265267 | 0 | −0.07253 | −0.06561 | −0.18622 | 0.866987 |
| 360 | −4.04298 | 0 | 0.063613 | 0.260216 | 0 | −0.03998 | −0.0716 | −0.22371 | 0.88287 |
| 361 | −4.08868 | 0 | 0.064091 | 0.277815 | 0 | −0.06124 | −0.07313 | −0.21228 | 0.880198 |
| 362 | −3.56943 | 0 | 0.054229 | 0.268059 | 0 | −0.07509 | −0.07243 | −0.15115 | 0.73324 |
| 363 | −3.52102 | 0 | 0.053653 | 0.260943 | 0 | −0.04309 | −0.07748 | −0.18947 | 0.743235 |
| 364 | −3.53468 | 0 | 0.053801 | 0.282501 | 0 | −0.06657 | −0.0791 | −0.18187 | 0.752256 |
| 365 | −3.7907 | 0 | 0.058666 | 0.254028 | 0 | −0.06601 | −0.07887 | −0.19875 | 0.797659 |
| 366 | −3.65671 | 0 | 0.056095 | 0.275052 | 0 | −0.12601 | −0.05868 | 0 | 0.834101 |
| 367 | −3.61845 | 0 | 0.055352 | 0.260181 | 0 | −0.09907 | −0.06584 | 0 | 0.815717 |
| 368 | −3.62777 | 0 | 0.055476 | 0.280623 | 0 | −0.12182 | −0.06451 | 0 | 0.826768 |
| 369 | −3.95297 | 0 | 0.061185 | 0.237841 | 0 | −0.11801 | −0.07587 | 0 | 0.841628 |
| 370 | −3.45395 | 0 | 0.052074 | 0.251365 | 0 | −0.11672 | −0.0791 | 0 | 0.74656 |
| 371 | −3.71743 | 0 | 0.057868 | 0.284084 | 0 | −0.08777 | 0 | −0.17277 | 0.854211 |
| 372 | −3.67982 | 0 | 0.057434 | 0.275329 | 0 | −0.05243 | 0 | −0.21435 | 0.859575 |
| 373 | −3.68021 | 0 | 0.057355 | 0.296194 | 0 | −0.07742 | 0 | −0.20438 | 0.866383 |
| 374 | −4.01547 | 0 | 0.063511 | 0.253937 | 0 | −0.07233 | 0 | −0.22344 | 0.904316 |
| 375 | −3.46356 | 0 | 0.053203 | 0.256005 | 0 | −0.07575 | 0 | −0.18888 | 0.778134 |
| 376 | −3.58369 | 0 | 0.055175 | 0.258704 | 0 | −0.13322 | 0 | 0 | 0.842595 |
| 377 | −3.75333 | 0 | 0.058077 | 0.296999 | 0 | 0 | −0.05732 | −0.18298 | 0.839051 |
| 378 | −3.70789 | 0 | 0.057609 | 0.28938 | 0 | 0 | −0.06205 | −0.21991 | 0.852219 |
| 379 | −3.72212 | 0 | 0.057677 | 0.310497 | 0 | 0 | −0.063 | −0.21351 | 0.854164 |
| 380 | −4.04337 | 0 | 0.063609 | 0.27062 | 0 | 0 | −0.07077 | −0.23022 | 0.892711 |
| 381 | −3.5073 | 0 | 0.053522 | 0.274294 | 0 | 0 | −0.07728 | −0.19763 | 0.76517 |
| 382 | −3.62207 | 0 | 0.055226 | 0.273445 | 0 | 0 | −0.06755 | 0 | 0.818916 |
| 383 | −3.68059 | 0 | 0.057428 | 0.288381 | 0 | 0 | 0 | −0.22231 | 0.87033 |
| 384 | −3.51236 | 0 | 0.053871 | 0 | 0.13428 | −0.07658 | −0.04211 | −0.15859 | 0.78363 |
| 385 | −3.47914 | 0 | 0.053395 | 0 | 0.143384 | −0.11369 | −0.04073 | −0.16299 | 0.795272 |
| 386 | −3.44124 | 0 | 0.052896 | 0 | 0.133182 | −0.07465 | −0.04644 | −0.19843 | 0.80146 |
| 387 | −3.80745 | 0 | 0.059406 | 0 | 0.126734 | −0.1046 | −0.04863 | −0.18142 | 0.839436 |
| 388 | −3.78805 | 0 | 0.059369 | 0 | 0.113786 | −0.07206 | −0.05348 | −0.21476 | 0.856454 |
| 389 | −3.81919 | 0 | 0.059587 | 0 | 0.132166 | −0.09627 | −0.05514 | −0.20611 | 0.848235 |
| 390 | −3.34839 | 0 | 0.050454 | 0 | 0.123404 | −0.10408 | −0.05338 | −0.15112 | 0.699126 |
| 391 | −3.30013 | 0 | 0.049903 | 0 | 0.110997 | −0.0724 | −0.05746 | −0.18459 | 0.713022 |
| 392 | −3.30483 | 0 | 0.049816 | 0 | 0.124963 | −0.09745 | −0.0583 | −0.17907 | 0.709608 |
| 393 | −3.56309 | 0 | 0.054839 | 0 | 0.111305 | −0.09554 | −0.05969 | −0.19423 | 0.771178 |
| 394 | −3.41415 | 0 | 0.052023 | 0 | 0.097196 | −0.15 | −0.03962 | 0 | 0.802287 |
| 395 | −3.38491 | 0 | 0.051461 | 0 | 0.079034 | −0.12165 | −0.04519 | 0 | 0.789437 |
| 396 | −3.38111 | 0 | 0.051322 | 0 | 0.089007 | −0.14607 | −0.04397 | 0 | 0.791973 |
| 397 | −3.73528 | 0 | 0.057594 | 0 | 0.070755 | −0.1381 | −0.05586 | 0 | 0.823101 |
| 398 | −3.25151 | 0 | 0.048615 | 0 | 0.073052 | −0.13551 | −0.05678 | 0 | 0.717151 |
| 399 | −3.45613 | 0 | 0.053404 | 0 | 0.126605 | −0.117 | 0 | −0.16704 | 0.816141 |
| 400 | −3.41865 | 0 | 0.052992 | 0 | 0.111886 | −0.0819 | 0 | −0.20391 | 0.825308 |
| 401 | −3.40666 | 0 | 0.052659 | 0 | 0.126422 | −0.10866 | 0 | −0.19595 | 0.823678 |
| 402 | −3.7698 | 0 | 0.059347 | 0 | 0.107478 | −0.10043 | 0 | −0.2143 | 0.875044 |
| 403 | −3.25314 | 0 | 0.049534 | 0 | 0.103093 | −0.10054 | 0 | −0.18394 | 0.743588 |
| 404 | −3.3562 | 0 | 0.051294 | 0 | 0.072686 | −0.15081 | 0 | 0 | 0.811249 |
| 405 | −3.48463 | 0 | 0.053444 | 0 | 0.109726 | 0 | −0.037 | −0.17939 | 0.798761 |
| 406 | −3.43672 | 0 | 0.052984 | 0 | 0.102882 | 0 | −0.04143 | −0.21283 | 0.816916 |
| 407 | −3.43868 | 0 | 0.052772 | 0 | 0.111355 | 0 | −0.04197 | −0.2077 | 0.808926 |
| 408 | −3.78738 | 0 | 0.059232 | 0 | 0.096408 | 0 | −0.04968 | −0.22329 | 0.860641 |
| 409 | −3.28272 | 0 | 0.049578 | 0 | 0.092801 | 0 | −0.05366 | −0.19453 | 0.726866 |
| 410 | −3.3803 | 0 | 0.05111 | 0 | 0.044632 | 0 | −0.04117 | 0 | 0.787686 |
| 411 | −3.41866 | 0 | 0.052841 | 0 | 0.092408 | 0 | 0 | −0.21354 | 0.829037 |
| 412 | −3.47202 | 0 | 0.053421 | 0 | 0 | −0.09399 | −0.0244 | −0.15411 | 0.813929 |
| 413 | −3.43429 | 0 | 0.05303 | 0 | 0 | −0.06131 | −0.0296 | −0.19237 | 0.826086 |
| 414 | −3.42551 | 0 | 0.052716 | 0 | 0 | −0.08547 | −0.02987 | −0.1817 | 0.821643 |
| 415 | −3.77572 | 0 | 0.059202 | 0 | 0 | −0.08109 | −0.03898 | −0.20143 | 0.875305 |
| 416 | −3.25563 | 0 | 0.049362 | 0 | 0 | −0.0821 | −0.04331 | −0.17153 | 0.746685 |
| 417 | −3.365 | 0 | 0.051284 | 0 | 0 | −0.13327 | −0.03266 | 0 | 0.810228 |
| 418 | −3.40654 | 0 | 0.052795 | 0 | 0 | −0.09137 | 0 | −0.18942 | 0.842432 |
| 419 | −3.42714 | 0 | 0.052827 | 0 | 0 | 0 | −0.02793 | −0.20065 | 0.830525 |
| 420 | −0.15902 | 0 | 0 | 0.157404 | 0.106323 | −0.05538 | −0.05842 | −0.13143 | 0.90565 |
| 421 | −0.15213 | 0 | 0 | 0.178728 | 0.109862 | −0.09921 | −0.05185 | −0.13829 | 0.928715 |
| 422 | −0.14806 | 0 | 0 | 0.170014 | 0.104715 | −0.05476 | −0.0599 | −0.17674 | 0.931277 |
| 423 | −0.16063 | 0 | 0 | 0.130693 | 0.092164 | −0.08432 | −0.07456 | −0.15337 | 0.900878 |
| 424 | −0.1474 | 0 | 0 | 0.123148 | 0.083916 | −0.05209 | −0.0796 | −0.1849 | 0.914426 |
| 425 | −0.16868 | 0 | 0 | 0.134403 | 0.09572 | −0.0747 | −0.07661 | −0.1778 | 0.904501 |
| 426 | −0.15687 | 0 | 0 | 0.16556 | 0.088219 | −0.09386 | −0.06933 | −0.13149 | 0.904029 |
| 427 | −0.14166 | 0 | 0 | 0.157694 | 0.077899 | −0.05575 | −0.07695 | −0.17199 | 0.917163 |
| 428 | −0.15051 | 0 | 0 | 0.175774 | 0.089312 | −0.08724 | −0.07323 | −0.16701 | 0.92408 |
| 429 | −0.14483 | 0 | 0 | 0.136173 | 0.076773 | −0.08084 | −0.08365 | −0.17739 | 0.914722 |
| 430 | −0.16965 | 0 | 0 | 0.162375 | 0.067245 | −0.12829 | −0.05539 | 0 | 0.926987 |
| 431 | −0.17779 | 0 | 0 | 0.148034 | 0.051697 | −0.09644 | −0.06236 | 0 | 0.914432 |
| 432 | −0.18166 | 0 | 0 | 0.166158 | 0.059431 | −0.12442 | −0.05666 | 0 | 0.923364 |
| 433 | −0.1951 | 0 | 0 | 0.115734 | 0.039851 | −0.10939 | −0.08 | 0 | 0.891022 |
| 434 | −0.17647 | 0 | 0 | 0.156706 | 0.038865 | −0.11998 | −0.07387 | 0 | 0.911726 |
| 435 | −0.12671 | 0 | 0 | 0.16307 | 0.092837 | −0.10419 | 0 | −0.1428 | 0.940349 |
| 436 | −0.11691 | 0 | 0 | 0.152616 | 0.082761 | −0.06593 | 0 | −0.18146 | 0.946514 |
| 437 | −0.12356 | 0 | 0 | 0.171501 | 0.093062 | −0.09603 | 0 | −0.17536 | 0.955026 |
| 438 | −0.12735 | 0 | 0 | 0.118573 | 0.072892 | −0.08349 | 0 | −0.1856 | 0.932156 |
| 439 | −0.11857 | 0 | 0 | 0.156565 | 0.067351 | −0.09144 | 0 | −0.17156 | 0.939344 |
| 440 | −0.15602 | 0 | 0 | 0.149026 | 0.041213 | −0.13112 | 0 | 0 | 0.935394 |
| 441 | −0.15437 | 0 | 0 | 0.177775 | 0.077054 | 0 | −0.05488 | −0.15193 | 0.921739 |
| 442 | −0.13695 | 0 | 0 | 0.167106 | 0.076406 | 0 | −0.061 | −0.18736 | 0.937215 |
| 443 | −0.14957 | 0 | 0 | 0.186368 | 0.078805 | 0 | −0.05586 | −0.18383 | 0.938661 |
| 444 | −0.15194 | 0 | 0 | 0.137147 | 0.064208 | 0 | −0.07823 | −0.1912 | 0.918904 |
| 445 | −0.14604 | 0 | 0 | 0.17346 | 0.056854 | 0 | −0.0735 | −0.18012 | 0.924431 |
| 446 | −0.19159 | 0 | 0 | 0.165497 | 0.016536 | 0 | −0.05966 | 0 | 0.911735 |
| 447 | −0.1232 | 0 | 0 | 0.169635 | 0.058854 | 0 | 0 | −0.18952 | 0.950569 |
| 448 | −0.14177 | 0 | 0 | 0.181548 | 0 | −0.08315 | −0.04519 | −0.1317 | 0.937291 |
| 449 | −0.13191 | 0 | 0 | 0.171446 | 0 | −0.04645 | −0.05229 | −0.17151 | 0.945167 |
| 450 | −0.13898 | 0 | 0 | 0.190267 | 0 | −0.07467 | −0.04648 | −0.16341 | 0.95176 |
| 451 | −0.1409 | 0 | 0 | 0.139613 | 0 | −0.0669 | −0.0712 | −0.17526 | 0.934764 |
| 452 | −0.13205 | 0 | 0 | 0.175606 | 0 | −0.07614 | −0.06697 | −0.16229 | 0.941644 |
| 453 | −0.16728 | 0 | 0 | 0.163922 | 0 | −0.11835 | −0.05373 | 0 | 0.932897 |
| 454 | −0.11192 | 0 | 0 | 0.172747 | 0 | −0.08359 | 0 | −0.17081 | 0.964648 |
| 455 | −0.13408 | 0 | 0 | 0.185093 | 0 | 0 | −0.05115 | −0.1796 | 0.950289 |
| 456 | −0.14839 | 0 | 0 | 0 | 0.118863 | −0.11359 | −0.03545 | −0.13628 | 0.91118 |
| 457 | −0.13683 | 0 | 0 | 0 | 0.108554 | −0.07625 | −0.04237 | −0.17326 | 0.92265 |
| 458 | −0.1463 | 0 | 0 | 0 | 0.119553 | −0.10645 | −0.03631 | −0.16703 | 0.922769 |
| 459 | −0.14406 | 0 | 0 | 0 | 0.097319 | −0.09258 | −0.06268 | −0.17842 | 0.916389 |
| 460 | −0.14419 | 0 | 0 | 0 | 0.093771 | −0.10035 | −0.05477 | −0.16484 | 0.9102 |
| 461 | −0.17405 | 0 | 0 | 0 | 0.067445 | −0.13827 | −0.04076 | 0 | 0.910704 |
| 462 | −0.1208 | 0 | 0 | 0 | 0.101754 | −0.11036 | 0 | −0.17214 | 0.938392 |
| 463 | −0.14661 | 0 | 0 | 0 | 0.084328 | 0 | −0.03767 | −0.18174 | 0.920575 |
| 464 | −5.41214 | 1.495741 | 0.068418 | 0.220937 | 0.046618 | −0.09305 | −0.07642 | −0.14925 | 0.537546 |
| 465 | −5.71828 | 1.38577 | 0.075439 | 0.221196 | 0.057119 | −0.08099 | −0.07312 | −0.17822 | 0.651559 |
| 466 | −5.56636 | 1.456412 | 0.07161 | 0.203983 | 0.03553 | −0.08924 | −0.07682 | −0.16389 | 0.555831 |
| 467 | −5.55394 | 1.430205 | 0.071715 | 0.217272 | 0.045803 | −0.11209 | −0.07842 | −0.16795 | 0.567206 |
| 468 | −5.53039 | 1.445092 | 0.071472 | 0.215482 | 0.035188 | −0.08537 | −0.08136 | −0.18898 | 0.59496 |
| 469 | −5.43109 | 1.427304 | 0.069349 | 0.19897 | −0.00732 | −0.14715 | −0.08027 | 0 | 0.580756 |
| 470 | −5.32993 | 1.399194 | 0.068659 | 0.230972 | 0.024518 | −0.13321 | −0.06406 | 0 | 0.684382 |
| 471 | −5.56602 | 1.370469 | 0.072697 | 0.196713 | 0.006061 | −0.1261 | −0.0723 | 0 | 0.650765 |
| 472 | −5.54962 | 1.348907 | 0.072607 | 0.208493 | 0.011227 | −0.15059 | −0.07298 | 0 | 0.656556 |
| 473 | −5.2612 | 1.479319 | 0.066097 | 0.201916 | 0.004243 | −0.13056 | −0.07319 | 0 | 0.581203 |
| 474 | −5.55656 | 1.375079 | 0.072421 | 0.201758 | −0.00203 | −0.12589 | −0.07674 | 0 | 0.651129 |
| 475 | −5.19996 | 1.451569 | 0.065463 | 0.21874 | 0.008082 | −0.15946 | −0.07294 | 0 | 0.595627 |
| 476 | −5.19831 | 1.479798 | 0.065114 | 0.211053 | −0.00643 | −0.13416 | −0.07622 | 0 | 0.591568 |
| 477 | −5.41182 | 1.419417 | 0.069261 | 0.194733 | −0.00316 | −0.15119 | −0.07591 | 0 | 0.588577 |
| 478 | −5.40808 | 1.438718 | 0.06905 | 0.188926 | −0.01747 | −0.13156 | −0.0787 | 0 | 0.59135 |
| 479 | −5.46063 | 1.422083 | 0.071031 | 0.210708 | 0.025226 | −0.11115 | 0 | −0.19057 | 0.628752 |
| 480 | −5.42367 | 1.406444 | 0.070895 | 0.242411 | 0.049501 | −0.09982 | 0 | −0.16928 | 0.702212 |
| 481 | −5.65399 | 1.374159 | 0.075083 | 0.207546 | 0.032321 | −0.09027 | 0 | −0.18491 | 0.687303 |
| 482 | −5.62947 | 1.348945 | 0.074945 | 0.221784 | 0.045613 | −0.11524 | 0 | −0.18881 | 0.698133 |
| 483 | −5.32989 | 1.486172 | 0.06781 | 0.206068 | 0.023746 | −0.09961 | 0 | −0.15348 | 0.584439 |
| 484 | −5.62552 | 1.366899 | 0.074956 | 0.2197 | 0.033025 | −0.08525 | 0 | −0.21241 | 0.719295 |
| 485 | −5.25585 | 1.456177 | 0.067047 | 0.224735 | 0.033498 | −0.12745 | 0 | −0.15696 | 0.603977 |
| 486 | −5.2281 | 1.477401 | 0.066603 | 0.221821 | 0.020189 | −0.09818 | 0 | −0.18085 | 0.622842 |
| 487 | −5.46537 | 1.422371 | 0.070998 | 0.202115 | 0.023403 | −0.11765 | 0 | −0.17269 | 0.613313 |
| 488 | −5.44178 | 1.43437 | 0.070824 | 0.200014 | 0.008686 | −0.09327 | 0 | −0.19523 | 0.641184 |
| 489 | −5.28801 | 1.389905 | 0.068445 | 0.216651 | 0.004566 | −0.14296 | 0 | 0 | 0.7034 |
| 490 | −5.21757 | 1.358416 | 0.06765 | 0.233926 | 0.009202 | −0.17467 | 0 | 0 | 0.713057 |
| 491 | −5.22146 | 1.391717 | 0.067326 | 0.2249 | −0.00734 | −0.14623 | 0 | 0 | 0.70442 |
| 492 | −5.4879 | 1.340918 | 0.072156 | 0.193364 | −0.00826 | −0.15856 | 0 | 0 | 0.6833 |
| 493 | −5.49082 | 1.363493 | 0.071992 | 0.1867 | −0.0247 | −0.1382 | 0 | 0 | 0.679638 |
| 494 | −5.49251 | 1.352266 | 0.071993 | 0.19745 | −0.01271 | −0.15352 | 0 | 0 | 0.678075 |
| 495 | −5.13633 | 1.445405 | 0.064967 | 0.202301 | −0.01065 | −0.16511 | 0 | 0 | 0.62632 |
| 496 | −5.13121 | 1.470153 | 0.064646 | 0.194956 | −0.02774 | −0.14281 | 0 | 0 | 0.62453 |
| 497 | −5.10474 | 1.453996 | 0.064304 | 0.209066 | −0.01792 | −0.1628 | 0 | 0 | 0.62477 |
| 498 | −5.3465 | 1.419476 | 0.068656 | 0.182739 | −0.02881 | −0.15455 | 0 | 0 | 0.62051 |
| 499 | −5.48132 | 1.404489 | 0.07106 | 0.228946 | 0.017396 | 0 | −0.08121 | −0.20198 | 0.615459 |
| 500 | −5.43359 | 1.389244 | 0.070842 | 0.255749 | 0.041232 | 0 | −0.06486 | −0.1795 | 0.694235 |
| 501 | −5.65587 | 1.359139 | 0.074914 | 0.222955 | 0.027957 | 0 | −0.07081 | −0.19339 | 0.681673 |
| 502 | −5.64373 | 1.333179 | 0.074864 | 0.237619 | 0.035454 | 0 | −0.07228 | −0.19962 | 0.683455 |
| 503 | −5.34101 | 1.469003 | 0.067751 | 0.223024 | 0.018113 | 0 | −0.07476 | −0.16435 | 0.575653 |
| 504 | −5.63275 | 1.350699 | 0.074887 | 0.235064 | 0.029088 | 0 | −0.07551 | −0.22059 | 0.714885 |
| 505 | −5.29184 | 1.437358 | 0.067259 | 0.241843 | 0.022056 | 0 | −0.0759 | −0.17121 | 0.583263 |
| 506 | −5.24363 | 1.458917 | 0.066636 | 0.239064 | 0.014681 | 0 | −0.0786 | −0.19194 | 0.616459 |
| 507 | −5.48241 | 1.406254 | 0.070937 | 0.220387 | 0.014846 | 0 | −0.07689 | −0.18478 | 0.597498 |
| 508 | −5.44631 | 1.418146 | 0.070688 | 0.217841 | 0.004899 | 0 | −0.07909 | −0.2051 | 0.636461 |
| 509 | −5.28176 | 1.36825 | 0.068031 | 0.231466 | −0.01478 | 0 | −0.06592 | 0 | 0.690675 |
| 510 | −5.22842 | 1.335875 | 0.067351 | 0.248114 | −0.01871 | 0 | −0.06558 | 0 | 0.687392 |
| 511 | −5.22354 | 1.369031 | 0.067038 | 0.240325 | −0.02741 | 0 | −0.06923 | 0 | 0.691755 |
| 512 | −5.48601 | 1.321433 | 0.07167 | 0.210927 | −0.02991 | 0 | −0.07411 | 0 | 0.661215 |
| 513 | −5.48515 | 1.342975 | 0.071571 | 0.204633 | −0.04071 | 0 | −0.07717 | 0 | 0.667774 |
| 514 | −5.49852 | 1.331704 | 0.071646 | 0.215176 | −0.03367 | 0 | −0.07794 | 0 | 0.65668 |
| 515 | −5.15836 | 1.422911 | 0.064824 | 0.220052 | −0.03372 | 0 | −0.07436 | 0 | 0.598718 |
| 516 | −5.13774 | 1.447798 | 0.064404 | 0.213353 | −0.04494 | 0 | −0.07706 | 0 | 0.610865 |
| 517 | −5.13364 | 1.4304 | 0.064295 | 0.226911 | −0.04024 | 0 | −0.07776 | 0 | 0.599081 |
| 518 | −5.35694 | 1.398597 | 0.06836 | 0.202303 | −0.04809 | 0 | −0.0806 | 0 | 0.597901 |
| 519 | −5.39264 | 1.38014 | 0.070649 | 0.241465 | 0.017438 | 0 | 0 | −0.18573 | 0.714103 |
| 520 | −5.32675 | 1.344818 | 0.069901 | 0.261347 | 0.022268 | 0 | 0 | −0.19385 | 0.719385 |
| 521 | −5.30992 | 1.370099 | 0.069662 | 0.25814 | 0.013848 | 0 | 0 | −0.21749 | 0.740601 |
| 522 | −5.58309 | 1.325839 | 0.074447 | 0.22291 | 0.012047 | 0 | 0 | −0.20641 | 0.711919 |
| 523 | −5.57217 | 1.339089 | 0.074535 | 0.220412 | 0.000357 | 0 | 0 | −0.2292 | 0.741614 |
| 524 | −5.57861 | 1.32723 | 0.074476 | 0.231858 | 0.015582 | 0 | 0 | −0.22444 | 0.730283 |
| 525 | −5.22313 | 1.431875 | 0.0667 | 0.225124 | 0.000387 | 0 | 0 | −0.17581 | 0.616401 |
| 526 | −5.17859 | 1.449707 | 0.066193 | 0.222156 | −0.01128 | 0 | 0 | −0.19783 | 0.648473 |
| 527 | −5.16624 | 1.431348 | 0.065964 | 0.236887 | −0.0005 | 0 | 0 | −0.19544 | 0.63752 |
| 528 | −5.397 | 1.39742 | 0.070376 | 0.212458 | −0.00834 | 0 | 0 | −0.20888 | 0.655265 |
| 529 | −5.18581 | 1.330565 | 0.067042 | 0.232246 | −0.03683 | 0 | 0 | 0 | 0.703314 |
| 530 | −5.1796 | 1.359436 | 0.066775 | 0.22474 | −0.04949 | 0 | 0 | 0 | 0.706999 |
| 531 | −5.1516 | 1.339015 | 0.066357 | 0.238647 | −0.04446 | 0 | 0 | 0 | 0.702617 |
| 532 | −5.43426 | 1.323481 | 0.071155 | 0.198533 | −0.05638 | 0 | 0 | 0 | 0.680995 |
| 533 | −5.06889 | 1.42437 | 0.063768 | 0.209066 | −0.06134 | 0 | 0 | 0 | 0.626669 |
| 534 | −5.49396 | 1.424731 | 0.071306 | 0.224707 | 0 | −0.10359 | −0.07611 | −0.18576 | 0.623251 |
| 535 | −5.46373 | 1.414491 | 0.071226 | 0.253601 | 0 | −0.08848 | −0.05731 | −0.16354 | 0.693939 |
| 536 | −5.68014 | 1.378729 | 0.075228 | 0.220447 | 0 | −0.08135 | −0.06479 | −0.17965 | 0.680426 |
| 537 | −5.66502 | 1.356548 | 0.07519 | 0.234679 | 0 | −0.1051 | −0.06571 | −0.18181 | 0.688687 |
| 538 | −5.36172 | 1.488904 | 0.068066 | 0.219395 | 0 | −0.09199 | −0.0694 | −0.14983 | 0.578467 |
| 539 | −5.65606 | 1.370973 | 0.07517 | 0.232634 | 0 | −0.07635 | −0.07003 | −0.20668 | 0.712912 |
| 540 | −5.29796 | 1.461116 | 0.06744 | 0.237937 | 0 | −0.11857 | −0.06953 | −0.15225 | 0.596816 |
| 541 | −5.26113 | 1.478883 | 0.066899 | 0.234984 | 0 | −0.09082 | −0.07352 | −0.17745 | 0.618494 |
| 542 | −5.49473 | 1.425408 | 0.071212 | 0.215811 | 0 | −0.11018 | −0.07142 | −0.16833 | 0.60716 |
| 543 | −5.46224 | 1.433807 | 0.070946 | 0.21328 | 0 | −0.08738 | −0.07495 | −0.19224 | 0.637214 |
| 544 | −5.31465 | 1.389943 | 0.068683 | 0.225807 | 0 | −0.13725 | −0.05968 | 0 | 0.696736 |
| 545 | −5.25117 | 1.359814 | 0.067994 | 0.242561 | 0 | −0.16849 | −0.05839 | 0 | 0.705824 |
| 546 | −5.24892 | 1.389049 | 0.067603 | 0.233268 | 0 | −0.14251 | −0.0638 | 0 | 0.698249 |
| 547 | −5.50885 | 1.338261 | 0.07232 | 0.203456 | 0 | −0.15526 | −0.06932 | 0 | 0.675949 |
| 548 | −5.50557 | 1.356926 | 0.07209 | 0.19617 | 0 | −0.13724 | −0.07388 | 0 | 0.672707 |
| 549 | −5.51781 | 1.348078 | 0.072231 | 0.20717 | 0 | −0.15125 | −0.07406 | 0 | 0.670494 |
| 550 | −5.16664 | 1.441716 | 0.065283 | 0.212794 | 0 | −0.16259 | −0.06984 | 0 | 0.618605 |
| 551 | −5.15385 | 1.4626 | 0.064869 | 0.204665 | 0 | −0.14297 | −0.07424 | 0 | 0.617035 |
| 552 | −5.13721 | 1.448168 | 0.064673 | 0.219153 | 0 | −0.16164 | −0.07395 | 0 | 0.617393 |
| 553 | −5.36714 | 1.411695 | 0.068841 | 0.192953 | 0 | −0.15501 | −0.07796 | 0 | 0.612129 |
| 554 | −5.41994 | 1.401279 | 0.071052 | 0.23938 | 0 | −0.09925 | 0 | −0.17041 | 0.716169 |
| 555 | −5.34935 | 1.370298 | 0.070304 | 0.259406 | 0 | −0.12889 | 0 | −0.17402 | 0.729306 |
| 556 | −5.3356 | 1.391614 | 0.070037 | 0.255482 | 0 | −0.09777 | 0 | −0.20234 | 0.742012 |
| 557 | −5.60253 | 1.34458 | 0.074803 | 0.219755 | 0 | −0.11384 | 0 | −0.18995 | 0.718255 |
| 558 | −5.58957 | 1.354379 | 0.074808 | 0.216745 | 0 | −0.08986 | 0 | −0.2164 | 0.741463 |
| 559 | −5.59875 | 1.347168 | 0.07481 | 0.228959 | 0 | −0.106 | 0 | −0.20818 | 0.734826 |
| 560 | −5.23027 | 1.451381 | 0.066943 | 0.22109 | 0 | −0.1259 | 0 | −0.15821 | 0.629711 |
| 561 | −5.19264 | 1.465057 | 0.066467 | 0.217191 | 0 | −0.10127 | 0 | −0.18447 | 0.65079 |
| 562 | −5.17334 | 1.451325 | 0.06618 | 0.232805 | 0 | −0.12078 | 0 | −0.1785 | 0.648277 |
| 563 | −5.40733 | 1.412621 | 0.070642 | 0.207303 | 0 | −0.11218 | 0 | −0.19428 | 0.662225 |
| 564 | −5.21075 | 1.351372 | 0.067755 | 0.227268 | 0 | −0.17764 | 0 | 0 | 0.722568 |
| 565 | −5.20315 | 1.376236 | 0.06737 | 0.217544 | 0 | −0.15636 | 0 | 0 | 0.715299 |
| 566 | −5.17131 | 1.35881 | 0.06698 | 0.232536 | 0 | −0.17589 | 0 | 0 | 0.719926 |
| 567 | −5.45204 | 1.336249 | 0.071767 | 0.189788 | 0 | −0.16356 | 0 | 0 | 0.694862 |
| 568 | −5.07406 | 1.4387 | 0.064208 | 0.200459 | 0 | −0.17125 | 0 | 0 | 0.643622 |
| 569 | −5.42264 | 1.383812 | 0.070906 | 0.251594 | 0 | 0 | −0.05888 | −0.18147 | 0.707871 |
| 570 | −5.36432 | 1.349748 | 0.070271 | 0.271259 | 0 | 0 | −0.05951 | −0.18902 | 0.71274 |
| 571 | −5.34342 | 1.372789 | 0.069984 | 0.268039 | 0 | 0 | −0.06331 | −0.21345 | 0.735256 |
| 572 | −5.60735 | 1.328205 | 0.074615 | 0.234529 | 0 | 0 | −0.06695 | −0.20187 | 0.705215 |
| 573 | −5.59022 | 1.338437 | 0.074639 | 0.231713 | 0 | 0 | −0.0702 | −0.22571 | 0.736239 |
| 574 | −5.60856 | 1.329517 | 0.074725 | 0.243638 | 0 | 0 | −0.07107 | −0.21925 | 0.723429 |
| 575 | −5.25561 | 1.431617 | 0.067024 | 0.237302 | 0 | 0 | −0.07172 | −0.17352 | 0.610425 |
| 576 | −5.20247 | 1.446542 | 0.066411 | 0.233927 | 0 | 0 | −0.07491 | −0.19661 | 0.643531 |
| 577 | −5.20155 | 1.430158 | 0.066344 | 0.249287 | 0 | 0 | −0.07579 | −0.1933 | 0.632681 |
| 578 | −5.41928 | 1.394803 | 0.070558 | 0.224928 | 0 | 0 | −0.07719 | −0.20693 | 0.649923 |
| 579 | −5.2112 | 1.323281 | 0.067338 | 0.239852 | 0 | 0 | −0.06597 | 0 | 0.696014 |
| 580 | −5.19909 | 1.349103 | 0.066989 | 0.231959 | 0 | 0 | −0.07054 | 0 | 0.699448 |
| 581 | −5.18032 | 1.329391 | 0.066715 | 0.245874 | 0 | 0 | −0.06991 | 0 | 0.694796 |
| 582 | −5.4496 | 1.311338 | 0.071298 | 0.206729 | 0 | 0 | −0.07955 | 0 | 0.671298 |
| 583 | −5.09726 | 1.410129 | 0.064117 | 0.217629 | 0 | 0 | −0.07998 | 0 | 0.615406 |
| 584 | −5.32209 | 1.341056 | 0.07002 | 0.255588 | 0 | 0 | 0 | −0.19609 | 0.730214 |
| 585 | −5.29799 | 1.358268 | 0.069773 | 0.251788 | 0 | 0 | 0 | −0.22218 | 0.7517 |
| 586 | −5.27157 | 1.340239 | 0.069362 | 0.267514 | 0 | 0 | 0 | −0.21815 | 0.747654 |
| 587 | −5.54378 | 1.316418 | 0.074318 | 0.226881 | 0 | 0 | 0 | −0.23055 | 0.749443 |
| 588 | −5.1353 | 1.420087 | 0.065851 | 0.229866 | 0 | 0 | 0 | −0.20139 | 0.659541 |
| 589 | −5.13194 | 1.320143 | 0.066367 | 0.22736 | 0 | 0 | 0 | 0 | 0.708633 |
| 590 | −5.31535 | 1.439169 | 0.068059 | 0 | 0.053741 | −0.12168 | −0.06071 | −0.17551 | 0.595105 |
| 591 | −5.2574 | 1.426075 | 0.067541 | 0 | 0.07605 | −0.1107 | −0.04484 | −0.15419 | 0.658948 |
| 592 | −5.49112 | 1.390485 | 0.071897 | 0 | 0.06152 | −0.10085 | −0.05066 | −0.17048 | 0.658876 |
| 593 | −5.46934 | 1.371351 | 0.071681 | 0 | 0.072733 | −0.12569 | −0.05243 | −0.17316 | 0.660494 |
| 594 | −5.19607 | 1.499202 | 0.065102 | 0 | 0.052708 | −0.10989 | −0.05397 | −0.14198 | 0.553904 |
| 595 | −5.45304 | 1.384832 | 0.071544 | 0 | 0.062453 | −0.09703 | −0.05632 | −0.19475 | 0.684775 |
| 596 | −5.13188 | 1.475612 | 0.064337 | 0 | 0.060151 | −0.13658 | −0.05407 | −0.14448 | 0.560111 |
| 597 | −5.08634 | 1.491783 | 0.063686 | 0 | 0.049206 | −0.10921 | −0.05737 | −0.16606 | 0.585017 |
| 598 | −5.32347 | 1.438075 | 0.068154 | 0 | 0.051916 | −0.12752 | −0.05574 | −0.15978 | 0.585759 |
| 599 | −5.28531 | 1.446171 | 0.067802 | 0 | 0.039336 | −0.10394 | −0.0583 | −0.18079 | 0.617804 |
| 600 | −5.14618 | 1.408336 | 0.065616 | 0 | 0.031068 | −0.14931 | −0.04406 | 0 | 0.669437 |
| 601 | −5.07639 | 1.383593 | 0.06473 | 0 | 0.033977 | −0.17983 | −0.04273 | 0 | 0.671115 |
| 602 | −5.07108 | 1.409838 | 0.064349 | 0 | 0.019341 | −0.15317 | −0.04759 | 0 | 0.668241 |
| 603 | −5.35498 | 1.359862 | 0.069503 | 0 | 0.018933 | −0.16452 | −0.05283 | 0 | 0.657739 |
| 604 | −5.34913 | 1.377176 | 0.069268 | 0 | 0.00413 | −0.1445 | −0.05654 | 0 | 0.658807 |
| 605 | −5.35825 | 1.370972 | 0.069296 | 0 | 0.014873 | −0.16019 | −0.05808 | 0 | 0.649877 |
| 606 | −5.02516 | 1.463598 | 0.062607 | 0 | 0.016128 | −0.17082 | −0.05187 | 0 | 0.592781 |
| 607 | −5.00576 | 1.482753 | 0.062143 | 0 | 0.000816 | −0.14939 | −0.05547 | 0 | 0.597702 |
| 608 | −4.99148 | 1.471945 | 0.061879 | 0 | 0.009318 | −0.16861 | −0.0557 | 0 | 0.587406 |
| 609 | −5.22207 | 1.433383 | 0.066194 | 0 | −0.00051 | −0.16074 | −0.05969 | 0 | 0.599081 |
| 610 | −5.22225 | 1.412096 | 0.067489 | 0 | 0.058291 | −0.11639 | 0 | −0.15937 | 0.683969 |
| 611 | −5.1442 | 1.385265 | 0.066522 | 0 | 0.067205 | −0.1462 | 0 | −0.16256 | 0.688195 |
| 612 | −5.1202 | 1.404133 | 0.066123 | 0 | 0.054323 | −0.11532 | 0 | −0.18727 | 0.703508 |
| 613 | −5.41644 | 1.357944 | 0.071434 | 0 | 0.054029 | −0.12969 | 0 | −0.17899 | 0.692661 |
| 614 | −5.39605 | 1.367092 | 0.071316 | 0 | 0.039576 | −0.10457 | 0 | −0.20237 | 0.717363 |
| 615 | −5.40173 | 1.361326 | 0.071222 | 0 | 0.05647 | −0.12295 | 0 | −0.19559 | 0.704016 |
| 616 | −5.0694 | 1.464062 | 0.063942 | 0 | 0.043505 | −0.1395 | 0 | −0.14861 | 0.599561 |
| 617 | −5.022 | 1.476665 | 0.063332 | 0 | 0.029081 | −0.11391 | 0 | −0.17165 | 0.624892 |
| 618 | −5.0061 | 1.465497 | 0.063001 | 0 | 0.041793 | −0.13458 | 0 | −0.16634 | 0.610865 |
| 619 | −5.23703 | 1.425321 | 0.067538 | 0 | 0.032762 | −0.12503 | 0 | −0.18203 | 0.640353 |
| 620 | −5.04798 | 1.373947 | 0.064676 | 0 | 0.020725 | −0.18389 | 0 | 0 | 0.691199 |
| 621 | −5.03585 | 1.396205 | 0.064264 | 0 | 0.003203 | −0.16066 | 0 | 0 | 0.688982 |
| 622 | −5.00162 | 1.382423 | 0.063766 | 0 | 0.012342 | −0.18111 | 0 | 0 | 0.686269 |
| 623 | −5.30353 | 1.357514 | 0.069006 | 0 | −0.00342 | −0.16639 | 0 | 0 | 0.678964 |
| 624 | −4.93516 | 1.46059 | 0.061542 | 0 | −0.00714 | −0.17272 | 0 | 0 | 0.62092 |
| 625 | −5.21763 | 1.396443 | 0.0672 | 0 | 0.045367 | 0 | −0.04242 | −0.17156 | 0.675704 |
| 626 | −5.15434 | 1.368403 | 0.066346 | 0 | 0.048409 | 0 | −0.04247 | −0.17804 | 0.670216 |
| 627 | −5.12012 | 1.38791 | 0.065909 | 0 | 0.041737 | 0 | −0.04671 | −0.19962 | 0.696642 |
| 628 | −5.41471 | 1.344892 | 0.071086 | 0 | 0.040397 | 0 | −0.0492 | −0.19145 | 0.678352 |
| 629 | −5.3894 | 1.35369 | 0.071004 | 0 | 0.030641 | 0 | −0.05203 | −0.2127 | 0.711946 |
| 630 | −5.40444 | 1.347631 | 0.070959 | 0 | 0.043574 | 0 | −0.05423 | −0.20729 | 0.691289 |
| 631 | −5.08551 | 1.447523 | 0.063825 | 0 | 0.028645 | 0 | −0.05174 | −0.16382 | 0.578166 |
| 632 | −5.02077 | 1.460739 | 0.063074 | 0 | 0.01881 | 0 | −0.05442 | −0.18413 | 0.616742 |
| 633 | −5.0244 | 1.448245 | 0.062947 | 0 | 0.027568 | 0 | −0.05553 | −0.18093 | 0.592861 |
| 634 | −5.23947 | 1.411062 | 0.067253 | 0 | 0.021224 | 0 | −0.05715 | −0.19467 | 0.626345 |
| 635 | −5.04265 | 1.352619 | 0.064117 | 0 | −0.00985 | 0 | −0.04359 | 0 | 0.665333 |
| 636 | −5.02351 | 1.375303 | 0.063721 | 0 | −0.02112 | 0 | −0.0478 | 0 | 0.674163 |
| 637 | −5.00279 | 1.360687 | 0.063324 | 0 | −0.01731 | 0 | −0.04734 | 0 | 0.661901 |
| 638 | −5.29191 | 1.339506 | 0.068353 | 0 | −0.02749 | 0 | −0.05685 | 0 | 0.6565 |
| 639 | −4.94429 | 1.439194 | 0.061198 | 0 | −0.0325 | 0 | −0.05504 | 0 | 0.594 |
| 640 | −5.12516 | 1.358935 | 0.066295 | 0 | 0.033214 | 0 | 0 | −0.18278 | 0.691456 |
| 641 | −5.08828 | 1.373582 | 0.065889 | 0 | 0.022156 | 0 | 0 | −0.206 | 0.717172 |
| 642 | −5.0613 | 1.359205 | 0.065374 | 0 | 0.031682 | 0 | 0 | −0.20212 | 0.704001 |
| 643 | −5.35188 | 1.333599 | 0.070734 | 0 | 0.02224 | 0 | 0 | −0.21548 | 0.721587 |
| 644 | −4.96559 | 1.436815 | 0.062597 | 0 | 0.009078 | 0 | 0 | −0.18678 | 0.628536 |
| 645 | −4.97049 | 1.350615 | 0.063218 | 0 | −0.03246 | 0 | 0 | 0 | 0.680666 |
| 646 | −5.25243 | 1.424182 | 0.067681 | 0 | 0 | −0.10636 | −0.03507 | −0.15291 | 0.678604 |
| 647 | −5.17931 | 1.399876 | 0.066757 | 0 | 0 | −0.1349 | −0.03478 | −0.15465 | 0.681478 |
| 648 | −5.15261 | 1.416122 | 0.066349 | 0 | 0 | −0.10558 | −0.0398 | −0.18065 | 0.698996 |
| 649 | −5.44124 | 1.369734 | 0.07152 | 0 | 0 | −0.12047 | −0.0427 | −0.1714 | 0.686198 |
| 650 | −5.41645 | 1.375659 | 0.071381 | 0 | 0 | −0.09689 | −0.04637 | −0.19621 | 0.713194 |
| 651 | −5.43092 | 1.373931 | 0.071354 | 0 | 0 | −0.11348 | −0.0484 | −0.18698 | 0.697275 |
| 652 | −5.09763 | 1.473767 | 0.064121 | 0 | 0 | −0.13164 | −0.0461 | −0.14275 | 0.594583 |
| 653 | −5.0432 | 1.483284 | 0.063443 | 0 | 0 | −0.10779 | −0.04983 | −0.1672 | 0.621977 |
| 654 | −5.03604 | 1.475115 | 0.063205 | 0 | 0 | −0.12691 | −0.05036 | −0.16028 | 0.606731 |
| 655 | −5.25736 | 1.432785 | 0.067611 | 0 | 0 | −0.11852 | −0.05247 | −0.17641 | 0.6366 |
| 656 | −5.07118 | 1.379833 | 0.06486 | 0 | 0 | −0.17784 | −0.03746 | 0 | 0.685858 |
| 657 | −5.05433 | 1.398303 | 0.064405 | 0 | 0 | −0.15735 | −0.04307 | 0 | 0.684277 |
| 658 | −5.02537 | 1.386611 | 0.063971 | 0 | 0 | −0.1764 | −0.04204 | 0 | 0.681287 |
| 659 | −5.31857 | 1.358229 | 0.069083 | 0 | 0 | −0.16414 | −0.05341 | 0 | 0.672965 |
| 660 | −4.95552 | 1.460193 | 0.061714 | 0 | 0 | −0.17158 | −0.05225 | 0 | 0.614613 |
| 661 | −5.15117 | 1.387449 | 0.066749 | 0 | 0 | −0.13949 | 0 | −0.16043 | 0.703245 |
| 662 | −5.11761 | 1.398346 | 0.066329 | 0 | 0 | −0.11366 | 0 | −0.18796 | 0.721155 |
| 663 | −5.08679 | 1.388365 | 0.065792 | 0 | 0 | −0.13383 | 0 | −0.18019 | 0.713614 |
| 664 | −5.37544 | 1.355485 | 0.071135 | 0 | 0 | −0.11979 | 0 | −0.19685 | 0.728268 |
| 665 | −4.97776 | 1.45981 | 0.062898 | 0 | 0 | −0.13168 | 0 | −0.16757 | 0.641356 |
| 666 | −4.9938 | 1.373891 | 0.063903 | 0 | 0 | −0.1833 | 0 | 0 | 0.70007 |
| 667 | −5.15013 | 1.367842 | 0.066477 | 0 | 0 | 0 | −0.03591 | −0.17726 | 0.687047 |
| 668 | −5.11073 | 1.380371 | 0.066051 | 0 | 0 | 0 | −0.03989 | −0.20161 | 0.713707 |
| 669 | −5.08911 | 1.36816 | 0.065593 | 0 | 0 | 0 | −0.04032 | −0.19643 | 0.699936 |
| 670 | −5.36991 | 1.340113 | 0.070806 | 0 | 0 | 0 | −0.04721 | −0.21004 | 0.716891 |
| 671 | −4.98663 | 1.440724 | 0.062749 | 0 | 0 | 0 | −0.05055 | −0.18368 | 0.624491 |
| 672 | −4.98645 | 1.346149 | 0.063377 | 0 | 0 | 0 | −0.04716 | 0 | 0.674066 |
| 673 | −5.0579 | 1.354486 | 0.065562 | 0 | 0 | 0 | 0 | −0.20432 | 0.719992 |
| 674 | −0.88211 | 1.127699 | 0 | 0.100072 | 0.029813 | −0.09405 | −0.07135 | −0.15126 | 0.7745 |
| 675 | −0.8557 | 1.096427 | 0 | 0.127122 | 0.057953 | −0.07312 | −0.04474 | −0.11508 | 0.809138 |
| 676 | −0.8686 | 1.062419 | 0 | 0.08473 | 0.040564 | −0.063 | −0.06604 | −0.13215 | 0.733096 |
| 677 | −0.86822 | 1.048879 | 0 | 0.097406 | 0.047712 | −0.09263 | −0.06257 | −0.13533 | 0.736513 |
| 678 | −0.86357 | 1.062227 | 0 | 0.089802 | 0.04159 | −0.0608 | −0.06834 | −0.15806 | 0.753579 |
| 679 | −0.89706 | 1.204494 | 0 | 0.11256 | 0.030564 | −0.07622 | −0.06102 | −0.11013 | 0.811652 |
| 680 | −0.88284 | 1.181488 | 0 | 0.13264 | 0.035903 | −0.11708 | −0.05356 | −0.1151 | 0.825814 |
| 681 | −0.87695 | 1.201976 | 0 | 0.124078 | 0.02761 | −0.07772 | −0.06293 | −0.14725 | 0.847608 |
| 682 | −0.87406 | 1.121416 | 0 | 0.097659 | 0.027376 | −0.10013 | −0.06982 | −0.13159 | 0.768887 |
| 683 | −0.85709 | 1.132886 | 0 | 0.090236 | 0.017163 | −0.07137 | −0.07623 | −0.15804 | 0.799069 |
| 684 | −0.86008 | 1.08949 | 0 | 0.111824 | 0.016467 | −0.10341 | −0.04741 | 0 | 0.816514 |
| 685 | −0.84526 | 1.069507 | 0 | 0.130545 | 0.017552 | −0.14381 | −0.04036 | 0 | 0.833585 |
| 686 | −0.86747 | 1.097162 | 0 | 0.116747 | 0.004334 | −0.11043 | −0.04824 | 0 | 0.825221 |
| 687 | −0.87363 | 1.0433 | 0 | 0.083407 | 0.000896 | −0.12124 | −0.0659 | 0 | 0.748696 |
| 688 | −0.88262 | 1.06154 | 0 | 0.072623 | −0.01248 | −0.09908 | −0.07035 | 0 | 0.751214 |
| 689 | −0.90175 | 1.061201 | 0 | 0.083323 | −0.0017 | −0.11667 | −0.06669 | 0 | 0.73682 |
| 690 | −0.88746 | 1.175347 | 0 | 0.117968 | −0.00466 | −0.14183 | −0.05599 | 0 | 0.834477 |
| 691 | −0.89876 | 1.200205 | 0 | 0.10519 | −0.02139 | −0.11394 | −0.06379 | 0 | 0.838918 |
| 692 | −0.90331 | 1.19071 | 0 | 0.121514 | −0.01103 | −0.14166 | −0.05709 | 0 | 0.837856 |
| 693 | −0.89902 | 1.129276 | 0 | 0.084238 | −0.0215 | −0.12553 | −0.07323 | 0 | 0.776967 |
| 694 | −0.82267 | 1.083411 | 0 | 0.113407 | 0.038804 | −0.08083 | 0 | −0.12086 | 0.823696 |
| 695 | −0.80328 | 1.060345 | 0 | 0.135339 | 0.045069 | −0.1202 | 0 | −0.12662 | 0.84582 |
| 696 | −0.80482 | 1.078991 | 0 | 0.124354 | 0.037086 | −0.08145 | 0 | −0.15624 | 0.853877 |
| 697 | −0.82132 | 1.033032 | 0 | 0.084499 | 0.028692 | −0.09827 | 0 | −0.14139 | 0.770137 |
| 698 | −0.80851 | 1.042724 | 0 | 0.076125 | 0.018314 | −0.07134 | 0 | −0.16557 | 0.792397 |
| 699 | −0.83131 | 1.042383 | 0 | 0.0874 | 0.031914 | −0.09147 | 0 | −0.15887 | 0.775953 |
| 700 | −0.84906 | 1.170255 | 0 | 0.117553 | 0.017629 | −0.11872 | 0 | −0.1207 | 0.842417 |
| 701 | −0.83742 | 1.187613 | 0 | 0.1075 | 0.004931 | −0.0844 | 0 | −0.1527 | 0.865686 |
| 702 | −0.84487 | 1.176997 | 0 | 0.125497 | 0.017216 | −0.11405 | 0 | −0.14709 | 0.863534 |
| 703 | −0.83111 | 1.114068 | 0 | 0.08604 | 0.007531 | −0.09869 | 0 | −0.1584 | 0.81172 |
| 704 | −0.82069 | 1.061127 | 0 | 0.117123 | 0.002694 | −0.14796 | 0 | 0 | 0.843052 |
| 705 | −0.83585 | 1.085441 | 0 | 0.102534 | −0.01411 | −0.12004 | 0 | 0 | 0.839161 |
| 706 | −0.83523 | 1.074632 | 0 | 0.120143 | −0.00459 | −0.14624 | 0 | 0 | 0.845344 |
| 707 | −0.8563 | 1.046773 | 0 | 0.068648 | −0.02225 | −0.12438 | 0 | 0 | 0.768323 |
| 708 | −0.87044 | 1.180319 | 0 | 0.105358 | −0.03078 | −0.14531 | 0 | 0 | 0.853571 |
| 709 | −0.83538 | 1.071848 | 0 | 0.127365 | 0.029177 | 0 | −0.04802 | −0.12869 | 0.815104 |
| 710 | −0.82351 | 1.047039 | 0 | 0.149686 | 0.025442 | 0 | −0.04173 | −0.13833 | 0.827417 |
| 711 | −0.81681 | 1.06659 | 0 | 0.138385 | 0.026449 | 0 | −0.04862 | −0.16434 | 0.84627 |
| 712 | −0.84069 | 1.022865 | 0 | 0.1027 | 0.01713 | 0 | −0.06582 | −0.14932 | 0.75606 |
| 713 | −0.82111 | 1.032275 | 0 | 0.093725 | 0.012902 | 0 | −0.07049 | −0.1716 | 0.786832 |
| 714 | −0.84876 | 1.031081 | 0 | 0.105379 | 0.020581 | 0 | −0.06635 | −0.16612 | 0.763144 |
| 715 | −0.87118 | 1.155247 | 0 | 0.134973 | 0.001876 | 0 | −0.05818 | −0.13301 | 0.823526 |
| 716 | −0.8512 | 1.174233 | 0 | 0.124993 | −0.00342 | 0 | −0.06564 | −0.16194 | 0.858949 |
| 717 | −0.86474 | 1.160756 | 0 | 0.142701 | 0.001625 | 0 | −0.05906 | −0.15893 | 0.846875 |
| 718 | −0.8502 | 1.102221 | 0 | 0.106014 | −0.00314 | 0 | −0.07453 | −0.16747 | 0.799838 |
| 719 | −0.84914 | 1.045762 | 0 | 0.133223 | −0.02581 | 0 | −0.04661 | 0 | 0.819131 |
| 720 | −0.85592 | 1.070248 | 0 | 0.11923 | −0.03495 | 0 | −0.05255 | 0 | 0.8254 |
| 721 | −0.86188 | 1.058143 | 0 | 0.13627 | −0.03301 | 0 | −0.04699 | 0 | 0.822415 |
| 722 | −0.8823 | 1.033623 | 0 | 0.088828 | −0.04185 | 0 | −0.07009 | 0 | 0.749234 |
| 723 | −0.89962 | 1.161854 | 0 | 0.124114 | −0.05544 | 0 | −0.06178 | 0 | 0.829845 |
| 724 | −0.7983 | 1.039386 | 0 | 0.136245 | 0.00961 | 0 | 0 | −0.14341 | 0.838002 |
| 725 | −0.78532 | 1.055013 | 0 | 0.124323 | 0.005026 | 0 | 0 | −0.17049 | 0.859494 |
| 726 | −0.79196 | 1.042616 | 0 | 0.143665 | 0.010056 | 0 | 0 | −0.16811 | 0.858315 |
| 727 | −0.80179 | 1.016351 | 0 | 0.091586 | −0.0016 | 0 | 0 | −0.17437 | 0.796031 |
| 728 | −0.83049 | 1.151692 | 0 | 0.126583 | −0.01863 | 0 | 0 | −0.16505 | 0.86464 |
| 729 | −0.83685 | 1.050838 | 0 | 0.121681 | −0.05047 | 0 | 0 | 0 | 0.832994 |
| 730 | −0.83757 | 1.089799 | 0 | 0.125323 | 0 | −0.07126 | −0.04231 | −0.11566 | 0.818986 |
| 731 | −0.81848 | 1.068568 | 0 | 0.145943 | 0 | −0.10983 | −0.03525 | −0.12043 | 0.840552 |
| 732 | −0.81959 | 1.085052 | 0 | 0.135708 | 0 | −0.07204 | −0.04346 | −0.15076 | 0.849316 |
| 733 | −0.83455 | 1.03693 | 0 | 0.098986 | 0 | −0.09053 | −0.06166 | −0.13632 | 0.767257 |
| 734 | −0.81998 | 1.044088 | 0 | 0.09059 | 0 | −0.06506 | −0.06727 | −0.16173 | 0.790891 |
| 735 | −0.84566 | 1.046607 | 0 | 0.102245 | 0 | −0.08328 | −0.06257 | −0.15319 | 0.772327 |
| 736 | −0.86012 | 1.171323 | 0 | 0.129214 | 0 | −0.11301 | −0.05421 | −0.11761 | 0.838779 |
| 737 | −0.84664 | 1.185654 | 0 | 0.11975 | 0 | −0.08053 | −0.06314 | −0.15099 | 0.863585 |
| 738 | −0.85581 | 1.177322 | 0 | 0.137101 | 0 | −0.10855 | −0.05552 | −0.14399 | 0.860062 |
| 739 | −0.84036 | 1.112859 | 0 | 0.100122 | 0 | −0.09472 | −0.07228 | −0.156 | 0.810991 |
| 740 | −0.82766 | 1.059565 | 0 | 0.124737 | 0 | −0.14459 | −0.04209 | 0 | 0.838597 |
| 741 | −0.84102 | 1.079679 | 0 | 0.109974 | 0 | −0.11985 | −0.05016 | 0 | 0.834551 |
| 742 | −0.84111 | 1.071155 | 0 | 0.126672 | 0 | −0.1445 | −0.04314 | 0 | 0.840665 |
| 743 | −0.86086 | 1.038585 | 0 | 0.078459 | 0 | −0.12596 | −0.06969 | 0 | 0.764267 |
| 744 | −0.87426 | 1.170094 | 0 | 0.112666 | 0 | −0.14874 | −0.06099 | 0 | 0.847456 |
| 745 | −0.79014 | 1.056471 | 0 | 0.132014 | 0 | −0.11533 | 0 | −0.12673 | 0.851427 |
| 746 | −0.78287 | 1.068564 | 0 | 0.120292 | 0 | −0.08368 | 0 | −0.15812 | 0.86434 |
| 747 | −0.78634 | 1.060492 | 0 | 0.139332 | 0 | −0.10946 | 0 | −0.15179 | 0.869261 |
| 748 | −0.79331 | 1.02623 | 0 | 0.086445 | 0 | −0.09288 | 0 | −0.16323 | 0.80597 |
| 749 | −0.818 | 1.162973 | 0 | 0.119322 | 0 | −0.11387 | 0 | −0.15175 | 0.876131 |
| 750 | −0.81236 | 1.05953 | 0 | 0.111022 | 0 | −0.15297 | 0 | 0 | 0.851342 |
| 751 | −0.8068 | 1.040168 | 0 | 0.14469 | 0 | 0 | −0.04291 | −0.14043 | 0.834063 |
| 752 | −0.79386 | 1.054524 | 0 | 0.133627 | 0 | 0 | −0.04899 | −0.1679 | 0.855862 |
| 753 | −0.80065 | 1.043316 | 0 | 0.151729 | 0 | 0 | −0.04286 | −0.16496 | 0.854232 |
| 754 | −0.80931 | 1.013661 | 0 | 0.103782 | 0 | 0 | −0.06816 | −0.17229 | 0.793835 |
| 755 | −0.83596 | 1.144675 | 0 | 0.135966 | 0 | 0 | −0.06239 | −0.16601 | 0.860224 |
| 756 | −0.83783 | 1.036364 | 0 | 0.125276 | 0 | 0 | −0.05329 | 0 | 0.825202 |
| 757 | −0.7707 | 1.031466 | 0 | 0.136285 | 0 | 0 | 0 | −0.17305 | 0.865551 |
| 758 | −0.84778 | 1.097152 | 0 | 0 | 0.058449 | −0.08872 | −0.03287 | −0.11564 | 0.802166 |
| 759 | −0.83327 | 1.078611 | 0 | 0 | 0.064894 | −0.12861 | −0.02556 | −0.12045 | 0.816826 |
| 760 | −0.8322 | 1.094877 | 0 | 0 | 0.056652 | −0.09048 | −0.03367 | −0.14912 | 0.829922 |
| 761 | −0.84221 | 1.043224 | 0 | 0 | 0.047816 | −0.10431 | −0.05322 | −0.13651 | 0.754985 |
| 762 | −0.82704 | 1.050891 | 0 | 0 | 0.03737 | −0.07726 | −0.05887 | −0.16041 | 0.781075 |
| 763 | −0.85313 | 1.052752 | 0 | 0 | 0.050838 | −0.09814 | −0.05438 | −0.15328 | 0.759088 |
| 764 | −0.87778 | 1.182913 | 0 | 0 | 0.038682 | −0.12603 | −0.04209 | −0.11583 | 0.816491 |
| 765 | −0.86345 | 1.198403 | 0 | 0 | 0.026182 | −0.09195 | −0.0506 | −0.14694 | 0.845584 |
| 766 | −0.87573 | 1.190697 | 0 | 0 | 0.038665 | −0.12202 | −0.0434 | −0.14068 | 0.834803 |
| 767 | −0.85288 | 1.123106 | 0 | 0 | 0.027688 | −0.10514 | −0.0616 | −0.15327 | 0.796932 |
| 768 | −0.84537 | 1.076664 | 0 | 0 | 0.022729 | −0.15393 | −0.02974 | 0 | 0.820037 |
| 769 | −0.85708 | 1.097426 | 0 | 0 | 0.005426 | −0.12587 | −0.03733 | 0 | 0.8212 |
| 770 | −0.85984 | 1.090511 | 0 | 0 | 0.015472 | −0.15266 | −0.03106 | 0 | 0.821823 |
| 771 | −0.87258 | 1.053449 | 0 | 0 | −0.00347 | −0.1286 | −0.05895 | 0 | 0.757449 |
| 772 | −0.89486 | 1.1914 | 0 | 0 | −0.00953 | −0.15063 | −0.04635 | 0 | 0.832263 |
| 773 | −0.80666 | 1.067174 | 0 | 0 | 0.050811 | −0.13025 | 0 | −0.12504 | 0.829792 |
| 774 | −0.7981 | 1.07976 | 0 | 0 | 0.038237 | −0.09694 | 0 | −0.15458 | 0.846945 |
| 775 | −0.80426 | 1.072608 | 0 | 0 | 0.050636 | −0.12526 | 0 | −0.14894 | 0.845571 |
| 776 | −0.8034 | 1.034559 | 0 | 0 | 0.029522 | −0.10265 | 0 | −0.16104 | 0.794959 |
| 777 | −0.83923 | 1.177454 | 0 | 0 | 0.019209 | −0.12265 | 0 | −0.14662 | 0.855674 |
| 778 | −0.83283 | 1.079241 | 0 | 0 | 1.86E−05 | −0.15611 | 0 | 0 | 0.835239 |
| 779 | −0.8287 | 1.056453 | 0 | 0 | 0.029984 | 0 | −0.02889 | −0.1377 | 0.810225 |
| 780 | −0.8125 | 1.069617 | 0 | 0 | 0.024773 | 0 | −0.03544 | −0.16409 | 0.837182 |
| 781 | −0.82399 | 1.061116 | 0 | 0 | 0.030042 | 0 | −0.02947 | −0.16099 | 0.828396 |
| 782 | −0.82411 | 1.026858 | 0 | 0 | 0.017864 | 0 | −0.05609 | −0.16896 | 0.780444 |
| 783 | −0.86124 | 1.164331 | 0 | 0 | 0.003017 | 0 | −0.04577 | −0.15907 | 0.837906 |
| 784 | −0.86228 | 1.06569 | 0 | 0 | −0.02962 | 0 | −0.03477 | 0 | 0.810073 |
| 785 | −0.79604 | 1.050414 | 0 | 0 | 0.014192 | 0 | 0 | −0.16675 | 0.842874 |
| 786 | −0.8224 | 1.079064 | 0 | 0 | 0 | −0.12016 | −0.02238 | −0.11817 | 0.825727 |
| 787 | −0.81233 | 1.088575 | 0 | 0 | 0 | −0.08868 | −0.03026 | −0.1489 | 0.843574 |
| 788 | −0.82033 | 1.084732 | 0 | 0 | 0 | −0.11515 | −0.02372 | −0.14163 | 0.841553 |
| 789 | −0.81745 | 1.041092 | 0 | 0 | 0 | −0.09591 | −0.05254 | −0.15526 | 0.792355 |
| 790 | −0.85038 | 1.18098 | 0 | 0 | 0 | −0.1179 | −0.04289 | −0.14294 | 0.852735 |
| 791 | −0.83874 | 1.078681 | 0 | 0 | 0 | −0.15437 | −0.03211 | 0 | 0.831405 |
| 792 | −0.78898 | 1.069168 | 0 | 0 | 0 | −0.11967 | 0 | −0.14882 | 0.85625 |
| 793 | −0.80445 | 1.054016 | 0 | 0 | 0 | 0 | −0.02919 | −0.16322 | 0.839885 |
| 794 | −0.13378 | 0 | 0 | 0 | 0 | −0.08901 | −0.02699 | −0.15915 | 0.936931 |
| 795 | −3.73365 | 0 | 0.055758 | 0.255711 | 0.122353 | −0.04625 | −0.09177 | −0.15141 | 0.61632 |
| 796 | −4.14406 | 0 | 0.06424 | 0.257775 | 0.125542 | −0.04114 | −0.08555 | −0.18087 | 0.805844 |
| 797 | −3.89094 | 0 | 0.059161 | 0.235066 | 0.106663 | −0.04888 | −0.08904 | −0.16544 | 0.681092 |
| 798 | −3.92756 | 0 | 0.059944 | 0.255436 | 0.118779 | −0.08046 | −0.09161 | −0.17229 | 0.714248 |
| 799 | −3.89776 | 0 | 0.059627 | 0.250097 | 0.109562 | −0.04442 | −0.09702 | −0.20412 | 0.724326 |
| 800 | −3.83802 | 0 | 0.058054 | 0.236834 | 0.064693 | −0.11426 | −0.09481 | 0 | 0.721503 |
| 801 | −3.71092 | 0 | 0.056428 | 0.266474 | 0.097183 | −0.09025 | −0.07623 | 0 | 0.789616 |
| 802 | −4.00451 | 0 | 0.061651 | 0.229827 | 0.074082 | −0.089 | −0.08337 | 0 | 0.805978 |
| 803 | −4.042 | 0 | 0.062231 | 0.247847 | 0.079915 | −0.12148 | −0.08357 | 0 | 0.819174 |
| 804 | −3.58772 | 0 | 0.053533 | 0.236185 | 0.078504 | −0.08594 | −0.08752 | 0 | 0.671564 |
| 805 | −4.02122 | 0 | 0.061734 | 0.235649 | 0.065765 | −0.08876 | −0.08954 | 0 | 0.799777 |
| 806 | −3.56288 | 0 | 0.053365 | 0.260311 | 0.083807 | −0.12249 | −0.08759 | 0 | 0.708647 |
| 807 | −3.55073 | 0 | 0.052907 | 0.245733 | 0.06812 | −0.09003 | −0.09266 | 0 | 0.676994 |
| 808 | −3.79378 | 0 | 0.05757 | 0.231194 | 0.068196 | −0.11985 | −0.08817 | 0 | 0.733642 |
| 809 | −3.77737 | 0 | 0.057199 | 0.219819 | 0.052474 | −0.09424 | −0.0926 | 0 | 0.717646 |
| 810 | −3.83944 | 0 | 0.059298 | 0.24027 | 0.096274 | −0.07824 | 0 | −0.20456 | 0.768337 |
| 811 | −3.75963 | 0 | 0.05801 | 0.26949 | 0.118466 | −0.05671 | 0 | −0.16958 | 0.802385 |
| 812 | −4.05888 | 0 | 0.063529 | 0.234398 | 0.096176 | −0.05327 | 0 | −0.18791 | 0.834952 |
| 813 | −4.08958 | 0 | 0.064058 | 0.253815 | 0.110646 | −0.08719 | 0 | −0.19343 | 0.854627 |
| 814 | −3.62191 | 0 | 0.054734 | 0.231303 | 0.095254 | −0.05487 | 0 | −0.15473 | 0.667802 |
| 815 | −4.06852 | 0 | 0.063901 | 0.24764 | 0.099626 | −0.04712 | 0 | −0.22754 | 0.863941 |
| 816 | −3.58315 | 0 | 0.054413 | 0.255774 | 0.106135 | −0.09084 | 0 | −0.15962 | 0.711959 |
| 817 | −3.54539 | 0 | 0.053882 | 0.247238 | 0.09432 | −0.05247 | 0 | −0.19523 | 0.709424 |
| 818 | −3.81302 | 0 | 0.058782 | 0.23036 | 0.091823 | −0.08704 | 0 | −0.17656 | 0.752788 |
| 819 | −3.78438 | 0 | 0.058573 | 0.223997 | 0.078194 | −0.05486 | 0 | −0.20984 | 0.766536 |
| 820 | −3.65721 | 0 | 0.056058 | 0.24408 | 0.072976 | −0.10174 | 0 | 0 | 0.810766 |
| 821 | −3.64482 | 0 | 0.055943 | 0.268115 | 0.078574 | −0.14035 | 0 | 0 | 0.830695 |
| 822 | −3.60961 | 0 | 0.055179 | 0.251261 | 0.06085 | −0.10456 | 0 | 0 | 0.805881 |
| 823 | −3.96067 | 0 | 0.061457 | 0.224291 | 0.056686 | −0.13015 | 0 | 0 | 0.841264 |
| 824 | −3.93986 | 0 | 0.061033 | 0.211353 | 0.038779 | −0.10321 | 0 | 0 | 0.823973 |
| 825 | −3.9802 | 0 | 0.061557 | 0.227736 | 0.052494 | −0.12338 | 0 | 0 | 0.831106 |
| 826 | −3.48114 | 0 | 0.052593 | 0.234822 | 0.060745 | −0.12849 | 0 | 0 | 0.736895 |
| 827 | −3.46106 | 0 | 0.052117 | 0.220256 | 0.042364 | −0.10021 | 0 | 0 | 0.713668 |
| 828 | −3.46548 | 0 | 0.052189 | 0.240483 | 0.053728 | −0.1246 | 0 | 0 | 0.728664 |
| 829 | −3.73116 | 0 | 0.056988 | 0.211009 | 0.038852 | −0.12232 | 0 | 0 | 0.755784 |
| 830 | −3.89074 | 0 | 0.059672 | 0.262872 | 0.094293 | 0 | −0.0942 | −0.21229 | 0.753835 |
| 831 | −3.79968 | 0 | 0.058326 | 0.285939 | 0.119371 | 0 | −0.0758 | −0.17535 | 0.793558 |
| 832 | −4.08514 | 0 | 0.063632 | 0.251378 | 0.098427 | 0 | −0.07974 | −0.19184 | 0.826897 |
| 833 | −4.13239 | 0 | 0.064297 | 0.273047 | 0.104992 | 0 | −0.08165 | −0.20098 | 0.837586 |
| 834 | −3.66138 | 0 | 0.055016 | 0.250032 | 0.098128 | 0 | −0.08636 | −0.16027 | 0.658079 |
| 835 | −4.10411 | 0 | 0.064146 | 0.266911 | 0.103264 | 0 | −0.08751 | −0.2315 | 0.85712 |
| 836 | −3.64622 | 0 | 0.054936 | 0.276302 | 0.101421 | 0 | −0.08825 | −0.1699 | 0.691964 |
| 837 | −3.58994 | 0 | 0.054256 | 0.268501 | 0.098237 | 0 | −0.09289 | −0.20134 | 0.703045 |
| 838 | −3.85846 | 0 | 0.059044 | 0.25061 | 0.087902 | 0 | −0.08655 | −0.18478 | 0.734687 |
| 839 | −3.81633 | 0 | 0.058744 | 0.244012 | 0.081208 | 0 | −0.09045 | −0.21499 | 0.759389 |
| 840 | −3.68509 | 0 | 0.056053 | 0.260539 | 0.061466 | 0 | −0.07378 | 0 | 0.797338 |
| 841 | −3.68883 | 0 | 0.056032 | 0.284323 | 0.056017 | 0 | −0.07254 | 0 | 0.805369 |
| 842 | −3.64712 | 0 | 0.055311 | 0.27001 | 0.048624 | 0 | −0.07831 | 0 | 0.792748 |
| 843 | −3.98857 | 0 | 0.061301 | 0.243213 | 0.038666 | 0 | −0.08094 | 0 | 0.817274 |
| 844 | −3.96414 | 0 | 0.060931 | 0.2315 | 0.028229 | 0 | −0.0857 | 0 | 0.80988 |
| 845 | −4.01742 | 0 | 0.061539 | 0.248826 | 0.035747 | 0 | −0.08686 | 0 | 0.80743 |
| 846 | −3.53313 | 0 | 0.052791 | 0.253955 | 0.04392 | 0 | −0.08489 | 0 | 0.709747 |
| 847 | −3.49897 | 0 | 0.052229 | 0.240904 | 0.033134 | 0 | −0.08867 | 0 | 0.699043 |
| 848 | −3.52344 | 0 | 0.052488 | 0.261683 | 0.038357 | 0 | −0.09021 | 0 | 0.703328 |
| 849 | −3.77217 | 0 | 0.057019 | 0.232556 | 0.024317 | 0 | −0.09088 | 0 | 0.731068 |
| 850 | −3.74742 | 0 | 0.057979 | 0.263518 | 0.090761 | 0 | 0 | −0.18145 | 0.815268 |
| 851 | −3.74331 | 0 | 0.057932 | 0.289954 | 0.094419 | 0 | 0 | −0.19118 | 0.831788 |
| 852 | −3.69339 | 0 | 0.057338 | 0.280784 | 0.090356 | 0 | 0 | −0.22559 | 0.840206 |
| 853 | −4.05038 | 0 | 0.063535 | 0.249966 | 0.077347 | 0 | 0 | −0.20782 | 0.861406 |
| 854 | −4.0273 | 0 | 0.063526 | 0.242985 | 0.068943 | 0 | 0 | −0.2405 | 0.880847 |
| 855 | −4.0716 | 0 | 0.06394 | 0.260178 | 0.084101 | 0 | 0 | −0.23441 | 0.87686 |
| 856 | −3.56079 | 0 | 0.054113 | 0.250489 | 0.075463 | 0 | 0 | −0.17376 | 0.721464 |
| 857 | −3.50464 | 0 | 0.053516 | 0.242059 | 0.067497 | 0 | 0 | −0.20635 | 0.737116 |
| 858 | −3.5221 | 0 | 0.053656 | 0.262981 | 0.077755 | 0 | 0 | −0.20408 | 0.742035 |
| 859 | −3.78274 | 0 | 0.058592 | 0.236538 | 0.063882 | 0 | 0 | −0.2188 | 0.787382 |
| 860 | −3.63821 | 0 | 0.055614 | 0.261715 | 0.034131 | 0 | 0 | 0 | 0.821187 |
| 861 | −3.59684 | 0 | 0.054955 | 0.246805 | 0.021905 | 0 | 0 | 0 | 0.810331 |
| 862 | −3.609 | 0 | 0.055012 | 0.267413 | 0.026915 | 0 | 0 | 0 | 0.814876 |
| 863 | −3.93805 | 0 | 0.060795 | 0.223863 | 0.008944 | 0 | 0 | 0 | 0.828206 |
| 864 | −3.44482 | 0 | 0.051747 | 0.235032 | 0.012901 | 0 | 0 | 0 | 0.729124 |
| 865 | −3.86978 | 0 | 0.05947 | 0.264951 | 0 | −0.05848 | −0.08404 | −0.19189 | 0.767217 |
| 866 | −3.79899 | 0 | 0.058299 | 0.289615 | 0 | −0.03268 | −0.06286 | −0.15783 | 0.795056 |
| 867 | −4.08516 | 0 | 0.063646 | 0.255287 | 0 | −0.03365 | −0.06919 | −0.17706 | 0.832239 |
| 868 | −4.1187 | 0 | 0.064183 | 0.275753 | 0 | −0.06502 | −0.07043 | −0.17994 | 0.847852 |
| 869 | −3.65102 | 0 | 0.054907 | 0.253542 | 0 | −0.03595 | −0.07568 | −0.14394 | 0.667089 |
| 870 | −4.10106 | 0 | 0.064095 | 0.271089 | 0 | −0.02652 | −0.07722 | −0.21538 | 0.861268 |
| 871 | −3.61774 | 0 | 0.054642 | 0.278555 | 0 | −0.06924 | −0.07659 | −0.14694 | 0.707465 |
| 872 | −3.57668 | 0 | 0.054094 | 0.271594 | 0 | −0.03314 | −0.08233 | −0.18418 | 0.710634 |
| 873 | −3.83855 | 0 | 0.058897 | 0.252282 | 0 | −0.06828 | −0.07633 | −0.16513 | 0.751051 |
| 874 | −3.80649 | 0 | 0.058669 | 0.246488 | 0 | −0.03832 | −0.08158 | −0.20022 | 0.769655 |
| 875 | −3.68758 | 0 | 0.056287 | 0.261977 | 0 | −0.08236 | −0.06357 | 0 | 0.805291 |
| 876 | −3.67602 | 0 | 0.05619 | 0.285157 | 0 | −0.11914 | −0.06167 | 0 | 0.823709 |
| 877 | −3.64139 | 0 | 0.055445 | 0.269713 | 0 | −0.08687 | −0.06934 | 0 | 0.801808 |
| 878 | −3.98236 | 0 | 0.061558 | 0.243119 | 0 | −0.11418 | −0.07284 | 0 | 0.83665 |
| 879 | −3.96089 | 0 | 0.061133 | 0.230592 | 0 | −0.09062 | −0.07933 | 0 | 0.821844 |
| 880 | −4.00793 | 0 | 0.061743 | 0.248157 | 0 | −0.1079 | −0.07965 | 0 | 0.826945 |
| 881 | −3.51043 | 0 | 0.052816 | 0.254781 | 0 | −0.1119 | −0.0764 | 0 | 0.732839 |
| 882 | −3.48561 | 0 | 0.052284 | 0.24061 | 0 | −0.08763 | −0.08222 | 0 | 0.712966 |
| 883 | −3.49632 | 0 | 0.052442 | 0.261927 | 0 | −0.10932 | −0.08262 | 0 | 0.726468 |
| 884 | −3.75444 | 0 | 0.057127 | 0.231359 | 0 | −0.1103 | −0.08481 | 0 | 0.754145 |
| 885 | −3.75083 | 0 | 0.058043 | 0.26852 | 0 | −0.04624 | 0 | −0.16514 | 0.819891 |
| 886 | −3.73209 | 0 | 0.057869 | 0.294094 | 0 | −0.0824 | 0 | −0.16831 | 0.842179 |
| 887 | −3.69464 | 0 | 0.057367 | 0.285309 | 0 | −0.04196 | 0 | −0.20873 | 0.843863 |
| 888 | −4.04292 | 0 | 0.063536 | 0.25306 | 0 | −0.07586 | 0 | −0.18902 | 0.872987 |
| 889 | −4.02659 | 0 | 0.063555 | 0.24634 | 0 | −0.04368 | 0 | −0.22649 | 0.88732 |
| 890 | −4.06305 | 0 | 0.063891 | 0.263883 | 0 | −0.06464 | 0 | −0.21489 | 0.88608 |
| 891 | −3.53998 | 0 | 0.053957 | 0.253762 | 0 | −0.07837 | 0 | −0.15317 | 0.738436 |
| 892 | −3.49582 | 0 | 0.053456 | 0.245261 | 0 | −0.04645 | 0 | −0.19144 | 0.746622 |
| 893 | −3.5012 | 0 | 0.053468 | 0.266345 | 0 | −0.06994 | 0 | −0.18353 | 0.755991 |
| 894 | −3.76805 | 0 | 0.058514 | 0.238449 | 0 | −0.06939 | 0 | −0.20137 | 0.801789 |
| 895 | −3.63227 | 0 | 0.055882 | 0.264152 | 0 | −0.12973 | 0 | 0 | 0.840583 |
| 896 | −3.59453 | 0 | 0.055168 | 0.247202 | 0 | −0.1033 | 0 | 0 | 0.821302 |
| 897 | −3.59815 | 0 | 0.055195 | 0.268457 | 0 | −0.12577 | 0 | 0 | 0.833059 |
| 898 | −3.93266 | 0 | 0.061077 | 0.222968 | 0 | −0.12206 | 0 | 0 | 0.847609 |
| 899 | −3.42427 | 0 | 0.051813 | 0.235287 | 0 | −0.12041 | 0 | 0 | 0.751681 |
| 900 | −3.7764 | 0 | 0.05819 | 0.281408 | 0 | 0 | −0.06014 | −0.16943 | 0.812144 |
| 901 | −3.77281 | 0 | 0.058156 | 0.30728 | 0 | 0 | −0.06062 | −0.17771 | 0.827433 |
| 902 | −3.72736 | 0 | 0.057627 | 0.299613 | 0 | 0 | −0.06597 | −0.2128 | 0.837472 |
| 903 | −4.07026 | 0 | 0.063613 | 0.268702 | 0 | 0 | −0.0681 | −0.19574 | 0.859246 |
| 904 | −4.04784 | 0 | 0.063619 | 0.262512 | 0 | 0 | −0.07303 | −0.22952 | 0.881168 |
| 905 | −4.09788 | 0 | 0.064095 | 0.281085 | 0 | 0 | −0.07511 | −0.22054 | 0.874351 |
| 906 | −3.58559 | 0 | 0.054285 | 0.270852 | 0 | 0 | −0.07489 | −0.16187 | 0.721938 |
| 907 | −3.52674 | 0 | 0.053656 | 0.263096 | 0 | 0 | −0.07887 | −0.19593 | 0.740318 |
| 908 | −3.54985 | 0 | 0.053869 | 0.285334 | 0 | 0 | −0.08122 | −0.19151 | 0.743671 |
| 909 | −3.80299 | 0 | 0.058697 | 0.257392 | 0 | 0 | −0.0809 | −0.2079 | 0.789596 |
| 910 | −3.6653 | 0 | 0.055861 | 0.277142 | 0 | 0 | −0.06435 | 0 | 0.816759 |
| 911 | −3.62326 | 0 | 0.055181 | 0.263048 | 0 | 0 | −0.07035 | 0 | 0.806809 |
| 912 | −3.64055 | 0 | 0.055323 | 0.283551 | 0 | 0 | −0.06992 | 0 | 0.810796 |
| 913 | −3.96069 | 0 | 0.060947 | 0.241028 | 0 | 0 | −0.08037 | 0 | 0.82393 |
| 914 | −3.47464 | 0 | 0.052024 | 0.253741 | 0 | 0 | −0.08333 | 0 | 0.726483 |
| 915 | −3.72979 | 0 | 0.057866 | 0.286251 | 0 | 0 | 0 | −0.18521 | 0.844548 |
| 916 | −3.68591 | 0 | 0.057438 | 0.277285 | 0 | 0 | 0 | −0.22232 | 0.856921 |
| 917 | −3.69251 | 0 | 0.05738 | 0.298612 | 0 | 0 | 0 | −0.21564 | 0.859356 |
| 918 | −4.02595 | 0 | 0.063523 | 0.256925 | 0 | 0 | 0 | −0.23351 | 0.897601 |
| 919 | −3.47946 | 0 | 0.053269 | 0.258402 | 0 | 0 | 0 | −0.20003 | 0.768515 |
| 920 | −3.59455 | 0 | 0.054975 | 0.259626 | 0 | 0 | 0 | 0 | 0.824504 |
| 921 | −3.63126 | 0 | 0.055442 | 0 | 0.127412 | −0.09082 | −0.0649 | −0.18929 | 0.735855 |
| 922 | −3.5305 | 0 | 0.053811 | 0 | 0.147646 | −0.06935 | −0.04701 | −0.15442 | 0.760799 |
| 923 | −3.83812 | 0 | 0.059556 | 0 | 0.128185 | −0.06615 | −0.05337 | −0.17264 | 0.808744 |
| 924 | −3.85297 | 0 | 0.059747 | 0 | 0.141285 | −0.0998 | −0.05367 | −0.17706 | 0.817341 |
| 925 | −3.43949 | 0 | 0.051348 | 0 | 0.125906 | −0.06582 | −0.05835 | −0.14411 | 0.640026 |
| 926 | −3.83392 | 0 | 0.059648 | 0 | 0.13232 | −0.06239 | −0.05972 | −0.20827 | 0.831687 |
| 927 | −3.39358 | 0 | 0.050769 | 0 | 0.135161 | −0.09995 | −0.05749 | −0.14856 | 0.666098 |
| 928 | −3.35348 | 0 | 0.050261 | 0 | 0.125719 | −0.06528 | −0.06239 | −0.18061 | 0.673283 |
| 929 | −3.6139 | 0 | 0.055139 | 0 | 0.122221 | −0.09823 | −0.05867 | −0.16415 | 0.726354 |
| 930 | −3.58289 | 0 | 0.054934 | 0 | 0.110254 | −0.06794 | −0.06317 | −0.19512 | 0.74668 |
| 931 | −3.45534 | 0 | 0.052417 | 0 | 0.102643 | −0.10839 | −0.04584 | 0 | 0.778504 |
| 932 | −3.4272 | 0 | 0.051988 | 0 | 0.106329 | −0.1457 | −0.04302 | 0 | 0.787228 |
| 933 | −3.40324 | 0 | 0.051456 | 0 | 0.090393 | −0.11306 | −0.04924 | 0 | 0.771273 |
| 934 | −3.7615 | 0 | 0.057901 | 0 | 0.087396 | −0.13693 | −0.0544 | 0 | 0.81621 |
| 935 | −3.74735 | 0 | 0.057622 | 0 | 0.070478 | −0.1111 | −0.0599 | 0 | 0.805923 |
| 936 | −3.78087 | 0 | 0.057975 | 0 | 0.08366 | −0.13149 | −0.06008 | 0 | 0.803996 |
| 937 | −3.30806 | 0 | 0.049347 | 0 | 0.090303 | −0.13351 | −0.05597 | 0 | 0.701575 |
| 938 | −3.28955 | 0 | 0.048965 | 0 | 0.073322 | −0.10741 | −0.06088 | 0 | 0.688795 |
| 939 | −3.29284 | 0 | 0.048912 | 0 | 0.084216 | −0.13041 | −0.06041 | 0 | 0.689129 |
| 940 | −3.55332 | 0 | 0.053773 | 0 | 0.069809 | −0.12938 | −0.06441 | 0 | 0.735462 |
| 941 | −3.50208 | 0 | 0.053838 | 0 | 0.127257 | −0.07667 | 0 | −0.15975 | 0.78824 |
| 942 | −3.46431 | 0 | 0.053278 | 0 | 0.137488 | −0.11383 | 0 | −0.16381 | 0.8 |
| 943 | −3.42753 | 0 | 0.052814 | 0 | 0.125984 | −0.07479 | 0 | −0.19933 | 0.805801 |
| 944 | −3.80079 | 0 | 0.059447 | 0 | 0.119089 | −0.10457 | 0 | −0.18327 | 0.844908 |
| 945 | −3.78399 | 0 | 0.059463 | 0 | 0.104752 | −0.07188 | 0 | −0.21677 | 0.861205 |
| 946 | −3.80726 | 0 | 0.059558 | 0 | 0.124409 | −0.09606 | 0 | −0.20784 | 0.85382 |
| 947 | −3.33438 | 0 | 0.050387 | 0 | 0.115019 | −0.10381 | 0 | −0.15229 | 0.704597 |
| 948 | −3.29064 | 0 | 0.049916 | 0 | 0.10151 | −0.07181 | 0 | −0.18589 | 0.717474 |
| 949 | −3.28823 | 0 | 0.049711 | 0 | 0.116305 | −0.0969 | 0 | −0.18002 | 0.714421 |
| 950 | −3.55556 | 0 | 0.054891 | 0 | 0.101821 | −0.09508 | 0 | −0.19605 | 0.776096 |
| 951 | −3.40245 | 0 | 0.051959 | 0 | 0.091066 | −0.15024 | 0 | 0 | 0.807375 |
| 952 | −3.37468 | 0 | 0.051437 | 0 | 0.071533 | −0.12185 | 0 | 0 | 0.794361 |
| 953 | −3.3666 | 0 | 0.051224 | 0 | 0.082546 | −0.14628 | 0 | 0 | 0.797114 |
| 954 | −3.72827 | 0 | 0.057649 | 0 | 0.061828 | −0.13814 | 0 | 0 | 0.828978 |
| 955 | −3.23831 | 0 | 0.048571 | 0 | 0.064043 | −0.13517 | 0 | 0 | 0.722944 |
| 956 | −3.51746 | 0 | 0.053793 | 0 | 0.119468 | 0 | −0.04227 | −0.16753 | 0.779444 |
| 957 | −3.49675 | 0 | 0.053372 | 0 | 0.121207 | 0 | −0.04106 | −0.17571 | 0.782258 |
| 958 | −3.4481 | 0 | 0.05285 | 0 | 0.118646 | 0 | −0.04665 | −0.20744 | 0.798174 |
| 959 | −3.818 | 0 | 0.059306 | 0 | 0.107147 | 0 | −0.04858 | −0.1922 | 0.828193 |
| 960 | −3.79428 | 0 | 0.059324 | 0 | 0.099982 | 0 | −0.05335 | −0.22301 | 0.853329 |
| 961 | −3.82942 | 0 | 0.059496 | 0 | 0.113726 | 0 | −0.05488 | −0.2161 | 0.8383 |
| 962 | −3.36707 | 0 | 0.050452 | 0 | 0.10402 | 0 | −0.05317 | −0.16294 | 0.683942 |
| 963 | −3.30615 | 0 | 0.049839 | 0 | 0.097277 | 0 | −0.05708 | −0.19329 | 0.708584 |
| 964 | −3.32287 | 0 | 0.049827 | 0 | 0.106555 | 0 | −0.05783 | −0.19011 | 0.696894 |
| 965 | −3.57605 | 0 | 0.054787 | 0 | 0.093507 | 0 | −0.0593 | −0.20453 | 0.759883 |
| 966 | −3.42516 | 0 | 0.051746 | 0 | 0.062748 | 0 | −0.04036 | 0 | 0.78285 |
| 967 | −3.38832 | 0 | 0.051205 | 0 | 0.051157 | 0 | −0.04565 | 0 | 0.779594 |
| 968 | −3.39397 | 0 | 0.051091 | 0 | 0.055322 | 0 | −0.04455 | 0 | 0.773733 |
| 969 | −3.73991 | 0 | 0.057243 | 0 | 0.039523 | 0 | −0.05595 | 0 | 0.804831 |
| 970 | −3.26814 | 0 | 0.048443 | 0 | 0.04296 | 0 | −0.05654 | 0 | 0.696583 |
| 971 | −3.47444 | 0 | 0.053393 | 0 | 0.103909 | 0 | 0 | −0.18031 | 0.803139 |
| 972 | −3.42821 | 0 | 0.052974 | 0 | 0.09585 | 0 | 0 | −0.21392 | 0.82109 |
| 973 | −3.42493 | 0 | 0.052674 | 0 | 0.105104 | 0 | 0 | −0.20851 | 0.813246 |
| 974 | −3.7826 | 0 | 0.059303 | 0 | 0.088476 | 0 | 0 | −0.22513 | 0.865672 |
| 975 | −3.27178 | 0 | 0.049556 | 0 | 0.08434 | 0 | 0 | −0.19561 | 0.731517 |
| 976 | −3.36972 | 0 | 0.051066 | 0 | 0.037933 | 0 | 0 | 0 | 0.792553 |
| 977 | −3.52166 | 0 | 0.053894 | 0 | 0 | −0.05402 | −0.02919 | −0.14713 | 0.785811 |
| 978 | −3.48547 | 0 | 0.053343 | 0 | 0 | −0.08898 | −0.0282 | −0.14887 | 0.795114 |
| 979 | −3.45009 | 0 | 0.052921 | 0 | 0 | −0.05208 | −0.03418 | −0.18541 | 0.803858 |
| 980 | −3.80884 | 0 | 0.05931 | 0 | 0 | −0.08363 | −0.037 | −0.1695 | 0.842659 |
| 981 | −3.7915 | 0 | 0.059342 | 0 | 0 | −0.053 | −0.04242 | −0.20437 | 0.862574 |
| 982 | −3.81931 | 0 | 0.059454 | 0 | 0 | −0.07402 | −0.04398 | −0.19169 | 0.850344 |
| 983 | −3.34142 | 0 | 0.050255 | 0 | 0 | −0.0835 | −0.04173 | −0.13872 | 0.704638 |
| 984 | −3.29258 | 0 | 0.049737 | 0 | 0 | −0.0539 | −0.04656 | −0.17387 | 0.721972 |
| 985 | −3.29781 | 0 | 0.04961 | 0 | 0 | −0.07634 | −0.04692 | −0.16519 | 0.714613 |
| 986 | −3.5567 | 0 | 0.054677 | 0 | 0 | −0.07701 | −0.04916 | −0.18298 | 0.778905 |
| 987 | −3.41548 | 0 | 0.05199 | 0 | 0 | −0.12919 | −0.03024 | 0 | 0.804197 |
| 988 | −3.3847 | 0 | 0.051437 | 0 | 0 | −0.1047 | −0.03712 | 0 | 0.793557 |
| 989 | −3.38007 | 0 | 0.051271 | 0 | 0 | −0.12656 | −0.03563 | 0 | 0.795104 |
| 990 | −3.73154 | 0 | 0.057519 | 0 | 0 | −0.12299 | −0.04889 | 0 | 0.828052 |
| 991 | −3.24132 | 0 | 0.048451 | 0 | 0 | −0.1201 | −0.0495 | 0 | 0.723336 |
| 992 | −3.46501 | 0 | 0.053386 | 0 | 0 | −0.09477 | 0 | −0.15507 | 0.816822 |
| 993 | −3.42782 | 0 | 0.053019 | 0 | 0 | −0.06226 | 0 | −0.19359 | 0.828981 |
| 994 | −3.41567 | 0 | 0.052648 | 0 | 0 | −0.08633 | 0 | −0.18272 | 0.824658 |
| 995 | −3.77191 | 0 | 0.059262 | 0 | 0 | −0.08213 | 0 | −0.20355 | 0.879087 |
| 996 | −3.24757 | 0 | 0.049359 | 0 | 0 | −0.083 | 0 | −0.1732 | 0.749976 |
| 997 | −3.35669 | 0 | 0.051254 | 0 | 0 | −0.13456 | 0 | 0 | 0.814004 |
| 998 | −3.48484 | 0 | 0.053391 | 0 | 0 | 0 | −0.02645 | −0.16632 | 0.802983 |
| 999 | −3.43979 | 0 | 0.052994 | 0 | 0 | 0 | −0.03094 | −0.20091 | 0.822497 |
| 1000 | −3.43817 | 0 | 0.052709 | 0 | 0 | 0 | −0.0316 | −0.19317 | 0.813072 |
| 1001 | −3.78492 | 0 | 0.059156 | 0 | 0 | 0 | −0.0404 | −0.21156 | 0.866916 |
| 1002 | −3.27078 | 0 | 0.049384 | 0 | 0 | 0 | −0.04454 | −0.18252 | 0.735681 |
| 1003 | −3.37704 | 0 | 0.051075 | 0 | 0 | 0 | −0.03635 | 0 | 0.791535 |
| 1004 | −3.42034 | 0 | 0.052803 | 0 | 0 | 0 | 0 | −0.20188 | 0.833372 |
| 1005 | −0.17777 | 0 | 0 | 0.139063 | 0.091931 | −0.07415 | −0.08399 | −0.17186 | 0.885191 |
| 1006 | −0.17947 | 0 | 0 | 0.164766 | 0.120027 | −0.04606 | −0.05842 | −0.1268 | 0.894271 |
| 1007 | −0.18351 | 0 | 0 | 0.120593 | 0.098292 | −0.04177 | −0.07588 | −0.14311 | 0.867416 |
| 1008 | −0.19015 | 0 | 0 | 0.133971 | 0.104495 | −0.07653 | −0.07353 | −0.14851 | 0.875718 |
| 1009 | −0.18442 | 0 | 0 | 0.151384 | 0.094888 | −0.0458 | −0.07411 | −0.12011 | 0.863728 |
| 1010 | −0.18241 | 0 | 0 | 0.126624 | 0.100128 | −0.03806 | −0.07984 | −0.17865 | 0.884213 |
| 1011 | −0.17698 | 0 | 0 | 0.17286 | 0.100798 | −0.09072 | −0.06977 | −0.12757 | 0.89164 |
| 1012 | −0.16614 | 0 | 0 | 0.16525 | 0.093384 | −0.04666 | −0.07875 | −0.1687 | 0.900182 |
| 1013 | −0.17256 | 0 | 0 | 0.13577 | 0.088066 | −0.08244 | −0.07987 | −0.14386 | 0.87896 |
| 1014 | −0.15542 | 0 | 0 | 0.128516 | 0.078988 | −0.04901 | −0.08643 | −0.17768 | 0.896441 |
| 1015 | −0.1868 | 0 | 0 | 0.149445 | 0.077492 | −0.07919 | −0.06044 | 0 | 0.903548 |
| 1016 | −0.1839 | 0 | 0 | 0.170137 | 0.077208 | −0.12355 | −0.05399 | 0 | 0.92135 |
| 1017 | −0.19612 | 0 | 0 | 0.155371 | 0.064213 | −0.08551 | −0.06199 | 0 | 0.904325 |
| 1018 | −0.20053 | 0 | 0 | 0.120019 | 0.055837 | −0.10719 | −0.07714 | 0 | 0.883784 |
| 1019 | −0.20536 | 0 | 0 | 0.107889 | 0.042499 | −0.08003 | −0.08182 | 0 | 0.875037 |
| 1020 | −0.22265 | 0 | 0 | 0.119821 | 0.053108 | −0.09991 | −0.07902 | 0 | 0.868086 |
| 1021 | −0.18359 | 0 | 0 | 0.159616 | 0.058399 | −0.11731 | −0.07066 | 0 | 0.903137 |
| 1022 | −0.18936 | 0 | 0 | 0.145962 | 0.041158 | −0.08565 | −0.07824 | 0 | 0.891961 |
| 1023 | −0.19503 | 0 | 0 | 0.164597 | 0.052381 | −0.11512 | −0.07447 | 0 | 0.90042 |
| 1024 | −0.19839 | 0 | 0 | 0.123199 | 0.037168 | −0.10693 | −0.08519 | 0 | 0.880169 |
| 1025 | −0.14793 | 0 | 0 | 0.148234 | 0.098726 | −0.05676 | 0 | −0.13257 | 0.910671 |
| 1026 | −0.14204 | 0 | 0 | 0.170927 | 0.103866 | −0.10057 | 0 | −0.13932 | 0.934364 |
| 1027 | −0.13639 | 0 | 0 | 0.160354 | 0.097245 | −0.05629 | 0 | −0.17761 | 0.936412 |
| 1028 | −0.14993 | 0 | 0 | 0.119275 | 0.083899 | −0.08523 | 0 | −0.15509 | 0.904077 |
| 1029 | −0.13602 | 0 | 0 | 0.110597 | 0.0745 | −0.05299 | 0 | −0.18636 | 0.916823 |
| 1030 | −0.15688 | 0 | 0 | 0.122108 | 0.087778 | −0.07576 | 0 | −0.1793 | 0.908364 |
| 1031 | −0.14508 | 0 | 0 | 0.155085 | 0.080579 | −0.09485 | 0 | −0.13256 | 0.908611 |
| 1032 | −0.12943 | 0 | 0 | 0.145615 | 0.068902 | −0.05641 | 0 | −0.17279 | 0.91986 |
| 1033 | −0.13821 | 0 | 0 | 0.163894 | 0.081182 | −0.08814 | 0 | −0.16781 | 0.927965 |
| 1034 | −0.13322 | 0 | 0 | 0.123323 | 0.067978 | −0.08143 | 0 | −0.17868 | 0.916554 |
| 1035 | −0.15935 | 0 | 0 | 0.153732 | 0.060299 | −0.1295 | 0 | 0 | 0.932337 |
| 1036 | −0.16637 | 0 | 0 | 0.137654 | 0.043453 | −0.09762 | 0 | 0 | 0.919344 |
| 1037 | −0.17069 | 0 | 0 | 0.157101 | 0.05234 | −0.12571 | 0 | 0 | 0.929098 |
| 1038 | −0.18359 | 0 | 0 | 0.102913 | 0.030523 | −0.11008 | 0 | 0 | 0.894163 |
| 1039 | −0.16429 | 0 | 0 | 0.144903 | 0.030043 | −0.12054 | 0 | 0 | 0.916044 |
| 1040 | −0.16682 | 0 | 0 | 0.161447 | 0.094297 | 0 | −0.05931 | −0.13713 | 0.901512 |
| 1041 | −0.16947 | 0 | 0 | 0.185202 | 0.088317 | 0 | −0.05365 | −0.14798 | 0.915279 |
| 1042 | −0.15542 | 0 | 0 | 0.174287 | 0.092677 | 0 | −0.06087 | −0.18253 | 0.927879 |
| 1043 | −0.17531 | 0 | 0 | 0.137037 | 0.074611 | 0 | −0.07516 | −0.16055 | 0.888892 |
| 1044 | −0.1547 | 0 | 0 | 0.127493 | 0.072768 | 0 | −0.07996 | −0.1897 | 0.910272 |
| 1045 | −0.18119 | 0 | 0 | 0.140345 | 0.079965 | 0 | −0.07721 | −0.18408 | 0.894628 |
| 1046 | −0.1742 | 0 | 0 | 0.170921 | 0.069082 | 0 | −0.07036 | −0.14106 | 0.889964 |
| 1047 | −0.1497 | 0 | 0 | 0.161515 | 0.066198 | 0 | −0.07739 | −0.17804 | 0.913015 |
| 1048 | −0.16608 | 0 | 0 | 0.180985 | 0.071395 | 0 | −0.07411 | −0.17581 | 0.912027 |
| 1049 | −0.15926 | 0 | 0 | 0.141957 | 0.060235 | 0 | −0.08403 | −0.18467 | 0.903232 |
| 1050 | −0.19502 | 0 | 0 | 0.169245 | 0.035476 | 0 | −0.05731 | 0 | 0.908032 |
| 1051 | −0.19383 | 0 | 0 | 0.153883 | 0.027597 | 0 | −0.06365 | 0 | 0.904921 |
| 1052 | −0.20529 | 0 | 0 | 0.173176 | 0.028573 | 0 | −0.05864 | 0 | 0.905893 |
| 1053 | −0.21665 | 0 | 0 | 0.122798 | 0.01398 | 0 | −0.08046 | 0 | 0.873784 |
| 1054 | −0.20107 | 0 | 0 | 0.162268 | 0.010685 | 0 | −0.07474 | 0 | 0.892793 |
| 1055 | −0.1444 | 0 | 0 | 0.169301 | 0.07012 | 0 | 0 | −0.15313 | 0.926694 |
| 1056 | −0.12594 | 0 | 0 | 0.157197 | 0.068091 | 0 | 0 | −0.18849 | 0.941708 |
| 1057 | −0.13897 | 0 | 0 | 0.177596 | 0.071816 | 0 | 0 | −0.18493 | 0.943965 |
| 1058 | −0.14115 | 0 | 0 | 0.12479 | 0.055185 | 0 | 0 | −0.19278 | 0.92138 |
| 1059 | −0.13442 | 0 | 0 | 0.16173 | 0.048418 | 0 | 0 | −0.18101 | 0.927749 |
| 1060 | −0.18082 | 0 | 0 | 0.155533 | 0.008449 | 0 | 0 | 0 | 0.916834 |
| 1061 | −0.16502 | 0 | 0 | 0.168089 | 0 | −0.03481 | −0.04945 | −0.12146 | 0.906896 |
| 1062 | −0.15858 | 0 | 0 | 0.190214 | 0 | −0.07738 | −0.04345 | −0.12651 | 0.92921 |
| 1063 | −0.15346 | 0 | 0 | 0.18057 | 0 | −0.03407 | −0.05149 | −0.1655 | 0.932684 |
| 1064 | −0.16455 | 0 | 0 | 0.140804 | 0 | −0.06682 | −0.06726 | −0.14352 | 0.904849 |
| 1065 | −0.15036 | 0 | 0 | 0.131963 | 0 | −0.03586 | −0.07267 | −0.17619 | 0.919565 |
| 1066 | −0.17299 | 0 | 0 | 0.145383 | 0 | −0.05632 | −0.06962 | −0.16654 | 0.908392 |
| 1067 | −0.16001 | 0 | 0 | 0.174405 | 0 | −0.07713 | −0.06273 | −0.12172 | 0.908666 |
| 1068 | −0.14341 | 0 | 0 | 0.165418 | 0 | −0.04085 | −0.0709 | −0.16363 | 0.92326 |
| 1069 | −0.15373 | 0 | 0 | 0.184922 | 0 | −0.07033 | −0.06665 | −0.15651 | 0.928602 |
| 1070 | −0.14654 | 0 | 0 | 0.144307 | 0 | −0.06631 | −0.07792 | −0.16867 | 0.921015 |
| 1071 | −0.17148 | 0 | 0 | 0.169912 | 0 | −0.11237 | −0.04971 | 0 | 0.928741 |
| 1072 | −0.17846 | 0 | 0 | 0.153705 | 0 | −0.08411 | −0.05786 | 0 | 0.91713 |
| 1073 | −0.18271 | 0 | 0 | 0.172926 | 0 | −0.11035 | −0.0518 | 0 | 0.925844 |
| 1074 | −0.19508 | 0 | 0 | 0.120587 | 0 | −0.1004 | −0.07673 | 0 | 0.89454 |
| 1075 | −0.17624 | 0 | 0 | 0.160968 | 0 | −0.1113 | −0.0708 | 0 | 0.91529 |
| 1076 | −0.13302 | 0 | 0 | 0.173828 | 0 | −0.08542 | 0 | −0.13324 | 0.941628 |
| 1077 | −0.12188 | 0 | 0 | 0.161956 | 0 | −0.0491 | 0 | −0.17314 | 0.949157 |
| 1078 | −0.12964 | 0 | 0 | 0.182327 | 0 | −0.07701 | 0 | −0.1649 | 0.956402 |
| 1079 | −0.13058 | 0 | 0 | 0.127145 | 0 | −0.0693 | 0 | −0.17765 | 0.936718 |
| 1080 | −0.12103 | 0 | 0 | 0.163937 | 0 | −0.07844 | 0 | −0.16396 | 0.944291 |
| 1081 | −0.15718 | 0 | 0 | 0.15421 | 0 | −0.12106 | 0 | 0 | 0.937476 |
| 1082 | −0.15576 | 0 | 0 | 0.185274 | 0 | 0 | −0.04816 | −0.14193 | 0.92565 |
| 1083 | −0.13817 | 0 | 0 | 0.174027 | 0 | 0 | −0.05397 | −0.17761 | 0.941392 |
| 1084 | −0.15088 | 0 | 0 | 0.193939 | 0 | 0 | −0.04916 | −0.17284 | 0.942521 |
| 1085 | −0.15195 | 0 | 0 | 0.143451 | 0 | 0 | −0.07282 | −0.18272 | 0.924939 |
| 1086 | −0.14536 | 0 | 0 | 0.178806 | 0 | 0 | −0.06894 | −0.17217 | 0.930544 |
| 1087 | −0.19098 | 0 | 0 | 0.167283 | 0 | 0 | −0.05809 | 0 | 0.913404 |
| 1088 | −0.12473 | 0 | 0 | 0.176049 | 0 | 0 | 0 | −0.18154 | 0.954211 |
| 1089 | −0.16838 | 0 | 0 | 0 | 0.123774 | −0.06637 | −0.04153 | −0.12639 | 0.886277 |
| 1090 | −0.16551 | 0 | 0 | 0 | 0.129862 | −0.11027 | −0.0342 | −0.13277 | 0.900459 |
| 1091 | −0.15832 | 0 | 0 | 0 | 0.123062 | −0.06732 | −0.04224 | −0.16909 | 0.908149 |
| 1092 | −0.1671 | 0 | 0 | 0 | 0.107933 | −0.09381 | −0.05967 | −0.14878 | 0.887074 |
| 1093 | −0.1519 | 0 | 0 | 0 | 0.098151 | −0.06192 | −0.06545 | −0.17933 | 0.904076 |
| 1094 | −0.17528 | 0 | 0 | 0 | 0.112396 | −0.08492 | −0.0614 | −0.17209 | 0.88918 |
| 1095 | −0.17103 | 0 | 0 | 0 | 0.106045 | −0.10288 | −0.05204 | −0.1277 | 0.877817 |
| 1096 | −0.15338 | 0 | 0 | 0 | 0.094708 | −0.06584 | −0.06005 | −0.16607 | 0.89573 |
| 1097 | −0.16694 | 0 | 0 | 0 | 0.10831 | −0.09704 | −0.05459 | −0.16099 | 0.892744 |
| 1098 | −0.15322 | 0 | 0 | 0 | 0.092302 | −0.09014 | −0.06851 | −0.17211 | 0.898433 |
| 1099 | −0.1787 | 0 | 0 | 0 | 0.086332 | −0.13662 | −0.03825 | 0 | 0.905086 |
| 1100 | −0.1838 | 0 | 0 | 0 | 0.068762 | −0.10504 | −0.0459 | 0 | 0.898277 |
| 1101 | −0.19011 | 0 | 0 | 0 | 0.079038 | −0.13329 | −0.03959 | 0 | 0.900357 |
| 1102 | −0.19802 | 0 | 0 | 0 | 0.054646 | −0.11666 | −0.06663 | 0 | 0.881668 |
| 1103 | −0.18725 | 0 | 0 | 0 | 0.056725 | −0.12675 | −0.0571 | 0 | 0.889272 |
| 1104 | −0.14121 | 0 | 0 | 0 | 0.113665 | −0.11383 | 0 | −0.13717 | 0.915476 |
| 1105 | −0.12835 | 0 | 0 | 0 | 0.101859 | −0.07642 | 0 | −0.1743 | 0.927022 |
| 1106 | −0.13884 | 0 | 0 | 0 | 0.114531 | −0.10671 | 0 | −0.1679 | 0.927201 |
| 1107 | −0.13434 | 0 | 0 | 0 | 0.088419 | −0.09223 | 0 | −0.18017 | 0.919818 |
| 1108 | −0.13417 | 0 | 0 | 0 | 0.085983 | −0.10005 | 0 | −0.16586 | 0.914525 |
| 1109 | −0.16593 | 0 | 0 | 0 | 0.061092 | −0.13844 | 0 | 0 | 0.915481 |
| 1110 | −0.16842 | 0 | 0 | 0 | 0.095295 | 0 | −0.03593 | −0.14692 | 0.895215 |
| 1111 | −0.14772 | 0 | 0 | 0 | 0.092437 | 0 | −0.04257 | −0.18088 | 0.916427 |
| 1112 | −0.16419 | 0 | 0 | 0 | 0.097295 | 0 | −0.03679 | −0.17698 | 0.909278 |
| 1113 | −0.15991 | 0 | 0 | 0 | 0.07859 | 0 | −0.06215 | −0.18584 | 0.903469 |
| 1114 | −0.16221 | 0 | 0 | 0 | 0.07383 | 0 | −0.05452 | −0.17452 | 0.89658 |
| 1115 | −0.20088 | 0 | 0 | 0 | 0.034406 | 0 | −0.04111 | 0 | 0.890116 |
| 1116 | −0.13908 | 0 | 0 | 0 | 0.078566 | 0 | 0 | −0.18267 | 0.924809 |
| 1117 | −5.31339 | 1.493782 | 0.06643 | 0.206354 | 0.01489 | −0.12492 | −0.07688 | 0 | 0.545744 |
| 1118 | −5.61525 | 1.386124 | 0.073006 | 0.201052 | 0.017969 | −0.11761 | −0.07637 | 0 | 0.624595 |
| 1119 | −5.486 | 1.454804 | 0.069726 | 0.18717 | 0.001783 | −0.12378 | −0.07875 | 0 | 0.545582 |
| 1120 | −5.47559 | 1.431903 | 0.069771 | 0.198932 | 0.006564 | −0.14651 | −0.07982 | 0 | 0.551681 |
| 1121 | −5.48154 | 1.454733 | 0.069622 | 0.192641 | −0.00569 | −0.12447 | −0.0829 | 0 | 0.549539 |
| 1122 | −5.38099 | 1.500031 | 0.068097 | 0.209907 | 0.036058 | −0.09533 | 0 | −0.14988 | 0.544781 |
| 1123 | −5.696 | 1.389569 | 0.075252 | 0.21134 | 0.047411 | −0.08341 | 0 | −0.17989 | 0.658958 |
| 1124 | −5.54559 | 1.460301 | 0.071458 | 0.192283 | 0.024144 | −0.09171 | 0 | −0.16511 | 0.562465 |
| 1125 | −5.52407 | 1.434124 | 0.071416 | 0.205878 | 0.035641 | −0.11425 | 0 | −0.16906 | 0.574697 |
| 1126 | −5.50737 | 1.450195 | 0.071268 | 0.203614 | 0.024179 | −0.08786 | 0 | −0.19012 | 0.60031 |
| 1127 | −5.4014 | 1.43172 | 0.069054 | 0.187335 | −0.01774 | −0.14967 | 0 | 0 | 0.58793 |
| 1128 | −5.30124 | 1.402131 | 0.068359 | 0.222794 | 0.015948 | −0.13632 | 0 | 0 | 0.691109 |
| 1129 | −5.54656 | 1.374008 | 0.072555 | 0.18622 | −0.00448 | −0.12933 | 0 | 0 | 0.657484 |
| 1130 | −5.2338 | 1.483284 | 0.065838 | 0.190901 | −0.00632 | −0.13322 | 0 | 0 | 0.58859 |
| 1131 | −5.52214 | 1.352003 | 0.072343 | 0.198675 | 0.002159 | −0.15326 | 0 | 0 | 0.664606 |
| 1132 | −5.53245 | 1.379167 | 0.072208 | 0.191321 | −0.01219 | −0.12903 | 0 | 0 | 0.657639 |
| 1133 | −5.16363 | 1.455324 | 0.065057 | 0.208203 | −0.00133 | −0.16196 | 0 | 0 | 0.603581 |
| 1134 | −5.16826 | 1.484308 | 0.064804 | 0.200017 | −0.01679 | −0.13698 | 0 | 0 | 0.597975 |
| 1135 | −5.38604 | 1.423128 | 0.069031 | 0.183177 | −0.01363 | −0.15388 | 0 | 0 | 0.596261 |
| 1136 | −5.38758 | 1.44319 | 0.068897 | 0.176923 | −0.02892 | −0.13462 | 0 | 0 | 0.597322 |
| 1137 | −5.39678 | 1.481956 | 0.068123 | 0.226734 | 0.031002 | 0 | −0.07847 | −0.16014 | 0.536425 |
| 1138 | −5.70306 | 1.373827 | 0.075178 | 0.226466 | 0.043854 | 0 | −0.07496 | −0.18745 | 0.654125 |
| 1139 | −5.55055 | 1.444148 | 0.07132 | 0.209816 | 0.020658 | 0 | −0.07884 | −0.17431 | 0.556549 |
| 1140 | −5.5482 | 1.417059 | 0.071461 | 0.223892 | 0.027134 | 0 | −0.0806 | −0.18058 | 0.557344 |
| 1141 | −5.51651 | 1.432806 | 0.07122 | 0.221268 | 0.021005 | 0 | −0.08327 | −0.19922 | 0.596458 |
| 1142 | −5.41797 | 1.409787 | 0.068862 | 0.206428 | −0.03674 | 0 | −0.08369 | 0 | 0.564408 |
| 1143 | −5.29879 | 1.379814 | 0.068029 | 0.23746 | −0.00255 | 0 | −0.06855 | 0 | 0.680048 |
| 1144 | −5.53686 | 1.353301 | 0.072112 | 0.203444 | −0.01896 | 0 | −0.07615 | 0 | 0.648211 |
| 1145 | −5.2342 | 1.460895 | 0.065532 | 0.20869 | −0.02195 | 0 | −0.0767 | 0 | 0.57654 |
| 1146 | −5.5266 | 1.331736 | 0.071963 | 0.215888 | −0.01941 | 0 | −0.0768 | 0 | 0.642038 |
| 1147 | −5.53097 | 1.35771 | 0.071886 | 0.208879 | −0.02721 | 0 | −0.08033 | 0 | 0.648016 |
| 1148 | −5.19317 | 1.431963 | 0.065035 | 0.225393 | −0.02441 | 0 | −0.07702 | 0 | 0.57476 |
| 1149 | −5.17799 | 1.460867 | 0.064638 | 0.218085 | −0.03324 | 0 | −0.07994 | 0 | 0.585424 |
| 1150 | −5.39611 | 1.402321 | 0.068717 | 0.202314 | −0.03341 | 0 | −0.07974 | 0 | 0.571786 |
| 1151 | −5.3866 | 1.421724 | 0.068549 | 0.196228 | −0.04364 | 0 | −0.08237 | 0 | 0.585631 |
| 1152 | −5.45242 | 1.408453 | 0.070768 | 0.216626 | 0.006363 | 0 | 0 | −0.2034 | 0.621429 |
| 1153 | −5.40519 | 1.391752 | 0.070545 | 0.247309 | 0.031807 | 0 | 0 | −0.18102 | 0.700674 |
| 1154 | −5.63832 | 1.361798 | 0.074801 | 0.212513 | 0.016908 | 0 | 0 | −0.19551 | 0.688274 |
| 1155 | −5.31397 | 1.472066 | 0.067497 | 0.211406 | 0.006515 | 0 | 0 | −0.16543 | 0.582231 |
| 1156 | −5.61723 | 1.335791 | 0.074611 | 0.227573 | 0.025985 | 0 | 0 | −0.20158 | 0.691209 |
| 1157 | −5.61096 | 1.354522 | 0.0747 | 0.224664 | 0.018515 | 0 | 0 | −0.22269 | 0.721128 |
| 1158 | −5.25506 | 1.440453 | 0.066843 | 0.230291 | 0.011531 | 0 | 0 | −0.17205 | 0.589943 |
| 1159 | −5.2141 | 1.462879 | 0.066326 | 0.227197 | 0.003278 | 0 | 0 | −0.19289 | 0.621424 |
| 1160 | −5.45724 | 1.409078 | 0.070713 | 0.208263 | 0.003539 | 0 | 0 | −0.18626 | 0.604054 |
| 1161 | −5.42792 | 1.421781 | 0.07056 | 0.205427 | −0.00736 | 0 | 0 | −0.20673 | 0.641359 |
| 1162 | −5.25411 | 1.369922 | 0.067755 | 0.221908 | −0.02517 | 0 | 0 | 0 | 0.69687 |
| 1163 | −5.19312 | 1.337143 | 0.066951 | 0.238938 | −0.02788 | 0 | 0 | 0 | 0.694076 |
| 1164 | −5.19178 | 1.370956 | 0.066701 | 0.230717 | −0.03772 | 0 | 0 | 0 | 0.6975 |
| 1165 | −5.46128 | 1.323339 | 0.071452 | 0.19957 | −0.0408 | 0 | 0 | 0 | 0.667983 |
| 1166 | −5.46351 | 1.345584 | 0.0714 | 0.192791 | −0.05289 | 0 | 0 | 0 | 0.673243 |
| 1167 | −5.46857 | 1.334262 | 0.071348 | 0.203922 | −0.04439 | 0 | 0 | 0 | 0.66363 |
| 1168 | −5.12425 | 1.425418 | 0.064458 | 0.208034 | −0.04461 | 0 | 0 | 0 | 0.605784 |
| 1169 | −5.10954 | 1.450745 | 0.06413 | 0.200972 | −0.05686 | 0 | 0 | 0 | 0.616447 |
| 1170 | −5.0965 | 1.433464 | 0.063876 | 0.21474 | −0.05126 | 0 | 0 | 0 | 0.605359 |
| 1171 | −5.33026 | 1.4016 | 0.068116 | 0.189279 | −0.0601 | 0 | 0 | 0 | 0.603961 |
| 1172 | −5.42297 | 1.504764 | 0.068468 | 0.223985 | 0 | −0.08632 | −0.0727 | −0.14504 | 0.536833 |
| 1173 | −5.73258 | 1.396828 | 0.075499 | 0.22515 | 0 | −0.07311 | −0.06878 | −0.17298 | 0.649185 |
| 1174 | −5.57405 | 1.463092 | 0.07165 | 0.206626 | 0 | −0.08414 | −0.07382 | −0.16076 | 0.556731 |
| 1175 | −5.56339 | 1.439397 | 0.071739 | 0.220376 | 0 | −0.10546 | −0.07492 | −0.16325 | 0.566685 |
| 1176 | −5.53812 | 1.452154 | 0.071507 | 0.218079 | 0 | −0.08031 | −0.07858 | −0.18553 | 0.595825 |
| 1177 | −5.42999 | 1.425765 | 0.069346 | 0.198388 | 0 | −0.14854 | −0.08086 | 0 | 0.580315 |
| 1178 | −5.33593 | 1.404199 | 0.068693 | 0.23249 | 0 | −0.12875 | −0.06213 | 0 | 0.683644 |
| 1179 | −5.56747 | 1.371686 | 0.072706 | 0.197173 | 0 | −0.12502 | −0.07178 | 0 | 0.650803 |
| 1180 | −5.26202 | 1.480163 | 0.066101 | 0.20223 | 0 | −0.12978 | −0.07283 | 0 | 0.581279 |
| 1181 | −5.55201 | 1.351295 | 0.072617 | 0.209322 | 0 | −0.14849 | −0.07212 | 0 | 0.656504 |
| 1182 | −5.55612 | 1.374661 | 0.072418 | 0.201597 | 0 | −0.12627 | −0.07691 | 0 | 0.651065 |
| 1183 | −5.20137 | 1.453292 | 0.065467 | 0.219284 | 0 | −0.15792 | −0.0723 | 0 | 0.595666 |
| 1184 | −5.1974 | 1.478467 | 0.065112 | 0.210572 | 0 | −0.13537 | −0.07675 | 0 | 0.591263 |
| 1185 | −5.41128 | 1.418767 | 0.069258 | 0.194489 | 0 | −0.15178 | −0.07617 | 0 | 0.588452 |
| 1186 | −5.40553 | 1.435238 | 0.069039 | 0.187488 | 0 | −0.13482 | −0.08021 | 0 | 0.589855 |
| 1187 | −5.4665 | 1.427035 | 0.071055 | 0.212912 | 0 | −0.10738 | 0 | −0.18791 | 0.629132 |
| 1188 | −5.43781 | 1.415659 | 0.070976 | 0.246056 | 0 | −0.09232 | 0 | −0.16523 | 0.700335 |
| 1189 | −5.66276 | 1.379981 | 0.075132 | 0.21042 | 0 | −0.08551 | 0 | −0.18214 | 0.687047 |
| 1190 | −5.33544 | 1.490503 | 0.067839 | 0.208171 | 0 | −0.09602 | 0 | −0.15146 | 0.584928 |
| 1191 | −5.64068 | 1.357679 | 0.074982 | 0.225495 | 0 | −0.10855 | 0 | −0.18422 | 0.696367 |
| 1192 | −5.63493 | 1.373229 | 0.07501 | 0.222604 | 0 | −0.08041 | 0 | −0.20922 | 0.719013 |
| 1193 | −5.26353 | 1.462684 | 0.067075 | 0.227341 | 0 | −0.12232 | 0 | −0.15366 | 0.603663 |
| 1194 | −5.23286 | 1.481326 | 0.066626 | 0.223551 | 0 | −0.09511 | 0 | −0.17897 | 0.623376 |
| 1195 | −5.47062 | 1.426733 | 0.071021 | 0.204189 | 0 | −0.11416 | 0 | −0.17045 | 0.613747 |
| 1196 | −5.44381 | 1.435987 | 0.070836 | 0.20084 | 0 | −0.09196 | 0 | −0.19446 | 0.64172 |
| 1197 | −5.28926 | 1.390789 | 0.068453 | 0.217024 | 0 | −0.14209 | 0 | 0 | 0.703335 |
| 1198 | −5.21987 | 1.360325 | 0.067662 | 0.234608 | 0 | −0.17284 | 0 | 0 | 0.712858 |
| 1199 | −5.21962 | 1.390244 | 0.067315 | 0.224301 | 0 | −0.14767 | 0 | 0 | 0.704363 |
| 1200 | −5.48588 | 1.339268 | 0.072145 | 0.192607 | 0 | −0.16014 | 0 | 0 | 0.683231 |
| 1201 | −5.48476 | 1.358756 | 0.07195 | 0.18429 | 0 | −0.14293 | 0 | 0 | 0.678423 |
| 1202 | −5.48948 | 1.349682 | 0.071975 | 0.196257 | 0 | −0.15599 | 0 | 0 | 0.677746 |
| 1203 | −5.13421 | 1.443272 | 0.064958 | 0.201355 | 0 | −0.16719 | 0 | 0 | 0.626091 |
| 1204 | −5.12668 | 1.464768 | 0.064628 | 0.192292 | 0 | −0.14821 | 0 | 0 | 0.622686 |
| 1205 | −5.10163 | 1.450314 | 0.064295 | 0.207463 | 0 | −0.16635 | 0 | 0 | 0.624001 |
| 1206 | −5.34143 | 1.413824 | 0.06863 | 0.179894 | 0 | −0.16013 | 0 | 0 | 0.6183 |
| 1207 | −5.48443 | 1.408341 | 0.071068 | 0.230086 | 0 | 0 | −0.07977 | −0.19974 | 0.616059 |
| 1208 | −5.44455 | 1.398117 | 0.070907 | 0.258057 | 0 | 0 | −0.06139 | −0.17528 | 0.693154 |
| 1209 | −5.66325 | 1.364878 | 0.07496 | 0.224839 | 0 | 0 | −0.0683 | −0.19046 | 0.681573 |
| 1210 | −5.34475 | 1.472828 | 0.06777 | 0.224221 | 0 | 0 | −0.07312 | −0.16239 | 0.576239 |
| 1211 | −5.65108 | 1.340699 | 0.074884 | 0.23985 | 0 | 0 | −0.06949 | −0.1953 | 0.682716 |
| 1212 | −5.64047 | 1.357094 | 0.074931 | 0.236998 | 0 | 0 | −0.0731 | −0.21718 | 0.714774 |
| 1213 | −5.29533 | 1.442209 | 0.067261 | 0.243138 | 0 | 0 | −0.07405 | −0.16848 | 0.583654 |
| 1214 | −5.24648 | 1.462238 | 0.066647 | 0.239987 | 0 | 0 | −0.07732 | −0.19022 | 0.617002 |
| 1215 | −5.48507 | 1.409347 | 0.070946 | 0.221382 | 0 | 0 | −0.07559 | −0.18302 | 0.598054 |
| 1216 | −5.44734 | 1.419191 | 0.070694 | 0.21819 | 0 | 0 | −0.07864 | −0.20456 | 0.636791 |
| 1217 | −5.27801 | 1.364893 | 0.068003 | 0.230518 | 0 | 0 | −0.06733 | 0 | 0.690626 |
| 1218 | −5.22482 | 1.331428 | 0.067333 | 0.246975 | 0 | 0 | −0.06726 | 0 | 0.687136 |
| 1219 | −5.21773 | 1.362559 | 0.067001 | 0.238574 | 0 | 0 | −0.07179 | 0 | 0.690959 |
| 1220 | −5.47976 | 1.314659 | 0.071628 | 0.208742 | 0 | 0 | −0.07688 | 0 | 0.660088 |
| 1221 | −5.4764 | 1.333906 | 0.071502 | 0.201538 | 0 | 0 | −0.08113 | 0 | 0.665049 |
| 1222 | −5.49205 | 1.323912 | 0.071604 | 0.212657 | 0 | 0 | −0.08092 | 0 | 0.654686 |
| 1223 | −5.15402 | 1.415044 | 0.064812 | 0.217666 | 0 | 0 | −0.07751 | 0 | 0.596672 |
| 1224 | −5.13269 | 1.43743 | 0.064391 | 0.209989 | 0 | 0 | −0.08143 | 0 | 0.606791 |
| 1225 | −5.12972 | 1.420753 | 0.064298 | 0.224042 | 0 | 0 | −0.08147 | 0 | 0.595788 |
| 1226 | −5.35128 | 1.387726 | 0.068332 | 0.198563 | 0 | 0 | −0.08516 | 0 | 0.59251 |
| 1227 | −5.39784 | 1.383718 | 0.070685 | 0.242851 | 0 | 0 | 0 | −0.18398 | 0.714025 |
| 1228 | −5.33231 | 1.349542 | 0.069928 | 0.262935 | 0 | 0 | 0 | −0.19131 | 0.719081 |
| 1229 | −5.31423 | 1.373139 | 0.069693 | 0.259183 | 0 | 0 | 0 | −0.21598 | 0.740618 |
| 1230 | −5.58603 | 1.328234 | 0.074462 | 0.223924 | 0 | 0 | 0 | −0.20502 | 0.711915 |
| 1231 | −5.57227 | 1.339162 | 0.074536 | 0.220443 | 0 | 0 | 0 | −0.22916 | 0.741624 |
| 1232 | −5.5825 | 1.330507 | 0.074495 | 0.233135 | 0 | 0 | 0 | −0.22246 | 0.730229 |
| 1233 | −5.2232 | 1.431955 | 0.0667 | 0.225157 | 0 | 0 | 0 | −0.17576 | 0.616416 |
| 1234 | −5.17616 | 1.447296 | 0.06618 | 0.22115 | 0 | 0 | 0 | −0.19908 | 0.647704 |
| 1235 | −5.16615 | 1.431238 | 0.065963 | 0.236846 | 0 | 0 | 0 | −0.1955 | 0.637499 |
| 1236 | −5.39524 | 1.395695 | 0.070367 | 0.211715 | 0 | 0 | 0 | −0.20988 | 0.654741 |
| 1237 | −5.17695 | 1.322222 | 0.066982 | 0.229134 | 0 | 0 | 0 | 0 | 0.702595 |
| 1238 | −5.16723 | 1.348389 | 0.066681 | 0.22034 | 0 | 0 | 0 | 0 | 0.704856 |
| 1239 | −5.14162 | 1.328683 | 0.066293 | 0.234929 | 0 | 0 | 0 | 0 | 0.700917 |
| 1240 | −5.42143 | 1.311231 | 0.071054 | 0.193063 | 0 | 0 | 0 | 0 | 0.67697 |
| 1241 | −5.06096 | 1.41046 | 0.063742 | 0.203131 | 0 | 0 | 0 | 0 | 0.620732 |
| 1242 | −5.26218 | 1.515033 | 0.06554 | 0 | 0.063699 | −0.10583 | −0.05861 | −0.13849 | 0.506135 |
| 1243 | −5.54133 | 1.408759 | 0.072116 | 0 | 0.075432 | −0.09469 | −0.05644 | −0.16535 | 0.624536 |
| 1244 | −5.40548 | 1.47311 | 0.068695 | 0 | 0.053604 | −0.10195 | −0.05903 | −0.15307 | 0.539628 |
| 1245 | −5.39193 | 1.45264 | 0.06864 | 0 | 0.063079 | −0.12417 | −0.06058 | −0.15603 | 0.540276 |
| 1246 | −5.36101 | 1.464516 | 0.068325 | 0 | 0.053661 | −0.09899 | −0.06371 | −0.17529 | 0.570601 |
| 1247 | −5.28533 | 1.447808 | 0.066654 | 0 | 0.010122 | −0.15616 | −0.06408 | 0 | 0.560692 |
| 1248 | −5.16568 | 1.422335 | 0.065569 | 0 | 0.041483 | −0.14336 | −0.04793 | 0 | 0.651688 |
| 1249 | −5.41426 | 1.389927 | 0.069935 | 0 | 0.023529 | −0.13579 | −0.05615 | 0 | 0.634021 |
| 1250 | −5.12361 | 1.497406 | 0.063541 | 0 | 0.021344 | −0.14001 | −0.05616 | 0 | 0.559196 |
| 1251 | −5.3949 | 1.373436 | 0.06971 | 0 | 0.02875 | −0.15983 | −0.05713 | 0 | 0.633397 |
| 1252 | −5.39733 | 1.394891 | 0.069525 | 0 | 0.016028 | −0.13623 | −0.06146 | 0 | 0.631965 |
| 1253 | −5.0637 | 1.475727 | 0.062792 | 0 | 0.024668 | −0.16754 | −0.05545 | 0 | 0.562454 |
| 1254 | −5.05353 | 1.498438 | 0.062405 | 0 | 0.011072 | −0.14389 | −0.05936 | 0 | 0.564791 |
| 1255 | −5.27031 | 1.439216 | 0.066669 | 0 | 0.01385 | −0.16006 | −0.05909 | 0 | 0.572277 |
| 1256 | −5.26047 | 1.454561 | 0.066428 | 0 | 1.16E−05 | −0.14107 | −0.06241 | 0 | 0.579244 |
| 1257 | −5.2958 | 1.439713 | 0.067911 | 0 | 0.046032 | −0.1222 | 0 | −0.17692 | 0.601263 |
| 1258 | −5.23831 | 1.426101 | 0.067368 | 0 | 0.070261 | −0.11176 | 0 | −0.15519 | 0.664094 |
| 1259 | −5.47919 | 1.390748 | 0.071853 | 0 | 0.054136 | −0.10186 | 0 | −0.17217 | 0.664958 |
| 1260 | −5.17731 | 1.499704 | 0.06496 | 0 | 0.044864 | −0.11058 | 0 | −0.14294 | 0.560332 |
| 1261 | −5.45157 | 1.371177 | 0.07155 | 0 | 0.066439 | −0.1264 | 0 | −0.17475 | 0.667187 |
| 1262 | −5.43761 | 1.385406 | 0.071448 | 0 | 0.055217 | −0.09796 | 0 | −0.1965 | 0.690362 |
| 1263 | −5.10678 | 1.475736 | 0.064093 | 0 | 0.053274 | −0.13722 | 0 | −0.14521 | 0.566745 |
| 1264 | −5.06603 | 1.492487 | 0.063515 | 0 | 0.041544 | −0.10996 | 0 | −0.16699 | 0.590267 |
| 1265 | −5.3068 | 1.438254 | 0.068051 | 0 | 0.04415 | −0.12826 | 0 | −0.1611 | 0.592449 |
| 1266 | −5.27328 | 1.447031 | 0.067766 | 0 | 0.030717 | −0.10483 | 0 | −0.18233 | 0.6232 |
| 1267 | −5.12857 | 1.408281 | 0.065468 | 0 | 0.024855 | −0.15072 | 0 | 0 | 0.674837 |
| 1268 | −5.05532 | 1.383175 | 0.064524 | 0 | 0.028705 | −0.18102 | 0 | 0 | 0.676714 |
| 1269 | −5.05098 | 1.409739 | 0.06417 | 0 | 0.013012 | −0.1547 | 0 | 0 | 0.673359 |
| 1270 | −5.33958 | 1.359805 | 0.069412 | 0 | 0.011723 | −0.16585 | 0 | 0 | 0.66433 |
| 1271 | −5.33558 | 1.377703 | 0.069208 | 0 | −0.00423 | −0.14616 | 0 | 0 | 0.66457 |
| 1272 | −5.33879 | 1.371052 | 0.06915 | 0 | 0.007543 | −0.1614 | 0 | 0 | 0.656449 |
| 1273 | −5.00281 | 1.463883 | 0.062406 | 0 | 0.009078 | −0.17181 | 0 | 0 | 0.599836 |
| 1274 | −4.98727 | 1.483499 | 0.062005 | 0 | −0.00726 | −0.15055 | 0 | 0 | 0.603661 |
| 1275 | −4.96683 | 1.472405 | 0.061644 | 0 | 0.002033 | −0.16957 | 0 | 0 | 0.593961 |
| 1276 | −5.20519 | 1.434088 | 0.066092 | 0 | −0.00899 | −0.1619 | 0 | 0 | 0.605697 |
| 1277 | −5.30391 | 1.424821 | 0.067704 | 0 | 0.034552 | 0 | −0.0615 | −0.18885 | 0.585707 |
| 1278 | −5.2367 | 1.410115 | 0.067132 | 0 | 0.058258 | 0 | −0.04667 | −0.16672 | 0.656613 |
| 1279 | −5.47099 | 1.377191 | 0.07152 | 0 | 0.045854 | 0 | −0.05201 | −0.18174 | 0.659463 |
| 1280 | −5.17537 | 1.483651 | 0.064688 | 0 | 0.035333 | 0 | −0.05504 | −0.15456 | 0.55067 |
| 1281 | −5.45468 | 1.357698 | 0.071268 | 0 | 0.052936 | 0 | −0.05351 | −0.18654 | 0.651712 |
| 1282 | −5.43438 | 1.371594 | 0.071197 | 0 | 0.047234 | 0 | −0.05743 | −0.20586 | 0.686082 |
| 1283 | −5.13007 | 1.458639 | 0.064079 | 0 | 0.038482 | 0 | −0.05531 | −0.16002 | 0.543644 |
| 1284 | −5.06805 | 1.47594 | 0.063314 | 0 | 0.031871 | 0 | −0.05852 | −0.17882 | 0.582456 |
| 1285 | −5.31177 | 1.423999 | 0.067772 | 0 | 0.032094 | 0 | −0.05688 | −0.17382 | 0.574524 |
| 1286 | −5.26634 | 1.432644 | 0.06743 | 0 | 0.022896 | 0 | −0.05944 | −0.19294 | 0.617296 |
| 1287 | −5.11049 | 1.387151 | 0.064872 | 0 | 0.0022 | 0 | −0.04738 | 0 | 0.66221 |
| 1288 | −5.05458 | 1.361275 | 0.06404 | 0 | −0.00175 | 0 | −0.04628 | 0 | 0.650653 |
| 1289 | −5.04049 | 1.388316 | 0.063675 | 0 | −0.0104 | 0 | −0.05104 | 0 | 0.660065 |
| 1290 | −5.3258 | 1.341645 | 0.068723 | 0 | −0.01278 | 0 | −0.05561 | 0 | 0.641165 |
| 1291 | −5.31791 | 1.358752 | 0.068582 | 0 | −0.02352 | 0 | −0.0594 | 0 | 0.651744 |
| 1292 | −5.33188 | 1.352545 | 0.068572 | 0 | −0.01628 | 0 | −0.06038 | 0 | 0.633593 |
| 1293 | −5.01153 | 1.442244 | 0.062041 | 0 | −0.01679 | 0 | −0.05437 | 0 | 0.570324 |
| 1294 | −4.97998 | 1.462276 | 0.061532 | 0 | −0.02775 | 0 | −0.05786 | 0 | 0.588405 |
| 1295 | −4.98213 | 1.45029 | 0.061399 | 0 | −0.02309 | 0 | −0.05802 | 0 | 0.565958 |
| 1296 | −5.20208 | 1.414843 | 0.065557 | 0 | −0.03112 | 0 | −0.06197 | 0 | 0.580993 |
| 1297 | −5.20116 | 1.395834 | 0.067066 | 0 | 0.039153 | 0 | 0 | −0.1728 | 0.680586 |
| 1298 | −5.13344 | 1.367493 | 0.066141 | 0 | 0.043012 | 0 | 0 | −0.17906 | 0.675202 |
| 1299 | −5.10055 | 1.387308 | 0.065732 | 0 | 0.035367 | 0 | 0 | −0.20083 | 0.700959 |
| 1300 | −5.40091 | 1.344185 | 0.071014 | 0 | 0.033517 | 0 | 0 | −0.19317 | 0.684319 |
| 1301 | −5.37815 | 1.353538 | 0.07097 | 0 | 0.022749 | 0 | 0 | −0.21469 | 0.717185 |
| 1302 | −5.38668 | 1.347249 | 0.07083 | 0 | 0.036776 | 0 | 0 | −0.2091 | 0.697272 |
| 1303 | −5.0641 | 1.447033 | 0.063638 | 0 | 0.021368 | 0 | 0 | −0.16476 | 0.584262 |
| 1304 | −5.00385 | 1.460654 | 0.062956 | 0 | 0.010707 | 0 | 0 | −0.18531 | 0.621655 |
| 1305 | −5.00073 | 1.447972 | 0.062724 | 0 | 0.020246 | 0 | 0 | −0.18188 | 0.598231 |
| 1306 | −5.22439 | 1.410948 | 0.067172 | 0 | 0.012972 | 0 | 0 | −0.19624 | 0.631963 |
| 1307 | −5.02238 | 1.351307 | 0.063931 | 0 | −0.01598 | 0 | 0 | 0 | 0.670668 |
| 1308 | −5.00415 | 1.374142 | 0.063559 | 0 | −0.02842 | 0 | 0 | 0 | 0.679 |
| 1309 | −4.97967 | 1.359381 | 0.063101 | 0 | −0.0238 | 0 | 0 | 0 | 0.667094 |
| 1310 | −5.27519 | 1.338644 | 0.068252 | 0 | −0.03592 | 0 | 0 | 0 | 0.662356 |
| 1311 | −4.92208 | 1.438693 | 0.061005 | 0 | −0.04055 | 0 | 0 | 0 | 0.600074 |
| 1312 | −5.32467 | 1.45012 | 0.068062 | 0 | 0 | −0.11419 | −0.0566 | −0.1694 | 0.595983 |
| 1313 | −5.27617 | 1.440583 | 0.067627 | 0 | 0 | −0.10028 | −0.03911 | −0.14727 | 0.656324 |
| 1314 | −5.50461 | 1.402025 | 0.071945 | 0 | 0 | −0.09263 | −0.04552 | −0.16477 | 0.658464 |
| 1315 | −5.20543 | 1.509128 | 0.065125 | 0 | 0 | −0.10263 | −0.04954 | −0.13702 | 0.555102 |
| 1316 | −5.48416 | 1.385866 | 0.071695 | 0 | 0 | −0.1158 | −0.04719 | −0.16523 | 0.657777 |
| 1317 | −5.46697 | 1.397323 | 0.071585 | 0 | 0 | −0.0886 | −0.05157 | −0.18818 | 0.684101 |
| 1318 | −5.14284 | 1.487623 | 0.064349 | 0 | 0 | −0.1281 | −0.04948 | −0.13802 | 0.559939 |
| 1319 | −5.09484 | 1.501652 | 0.063698 | 0 | 0 | −0.10232 | −0.05345 | −0.16102 | 0.586391 |
| 1320 | −5.33228 | 1.448097 | 0.068164 | 0 | 0 | −0.1204 | −0.05151 | −0.15437 | 0.586894 |
| 1321 | −5.29178 | 1.453748 | 0.067817 | 0 | 0 | −0.09846 | −0.05492 | −0.17697 | 0.62015 |
| 1322 | −5.15367 | 1.414599 | 0.065661 | 0 | 0 | −0.14386 | −0.04144 | 0 | 0.669179 |
| 1323 | −5.08415 | 1.390858 | 0.064768 | 0 | 0 | −0.1735 | −0.0401 | 0 | 0.67062 |
| 1324 | −5.07506 | 1.413851 | 0.064368 | 0 | 0 | −0.14962 | −0.046 | 0 | 0.668509 |
| 1325 | −5.35886 | 1.363843 | 0.06952 | 0 | 0 | −0.16112 | −0.05124 | 0 | 0.65799 |
| 1326 | −5.3499 | 1.37801 | 0.069272 | 0 | 0 | −0.14376 | −0.05617 | 0 | 0.65902 |
| 1327 | −5.36106 | 1.374152 | 0.069306 | 0 | 0 | −0.15745 | −0.0569 | 0 | 0.650301 |
| 1328 | −5.02766 | 1.466966 | 0.062612 | 0 | 0 | −0.16787 | −0.05052 | 0 | 0.593237 |
| 1329 | −5.00584 | 1.482918 | 0.062143 | 0 | 0 | −0.14924 | −0.0554 | 0 | 0.59776 |
| 1330 | −4.99263 | 1.473935 | 0.061878 | 0 | 0 | −0.16688 | −0.05494 | 0 | 0.587859 |
| 1331 | −5.22201 | 1.433279 | 0.066194 | 0 | 0 | −0.16083 | −0.05973 | 0 | 0.59904 |
| 1332 | −5.23773 | 1.423029 | 0.067568 | 0 | 0 | −0.10802 | 0 | −0.15425 | 0.683025 |
| 1333 | −5.16169 | 1.398564 | 0.066594 | 0 | 0 | −0.13637 | 0 | −0.1558 | 0.685957 |
| 1334 | −5.13526 | 1.414935 | 0.066202 | 0 | 0 | −0.10746 | 0 | −0.18203 | 0.703015 |
| 1335 | −5.42836 | 1.36828 | 0.07146 | 0 | 0 | −0.12216 | 0 | −0.17341 | 0.691811 |
| 1336 | −5.40532 | 1.374611 | 0.071353 | 0 | 0 | −0.09895 | 0 | −0.19851 | 0.718112 |
| 1337 | −5.41458 | 1.372668 | 0.071248 | 0 | 0 | −0.11502 | 0 | −0.18916 | 0.702858 |
| 1338 | −5.07758 | 1.472457 | 0.063955 | 0 | 0 | −0.13327 | 0 | −0.14412 | 0.600255 |
| 1339 | −5.02693 | 1.48228 | 0.063339 | 0 | 0 | −0.1097 | 0 | −0.16885 | 0.62644 |
| 1340 | −5.01404 | 1.473969 | 0.06301 | 0 | 0 | −0.12851 | 0 | −0.16173 | 0.611711 |
| 1341 | −5.24275 | 1.431655 | 0.067544 | 0 | 0 | −0.12037 | 0 | −0.17855 | 0.641814 |
| 1342 | −5.05329 | 1.378303 | 0.064705 | 0 | 0 | −0.17996 | 0 | 0 | 0.690986 |
| 1343 | −5.03658 | 1.396854 | 0.064268 | 0 | 0 | −0.16005 | 0 | 0 | 0.689054 |
| 1344 | −5.00454 | 1.385057 | 0.063781 | 0 | 0 | −0.17872 | 0 | 0 | 0.686367 |
| 1345 | −5.30283 | 1.356812 | 0.069004 | 0 | 0 | −0.16703 | 0 | 0 | 0.678825 |
| 1346 | −4.93423 | 1.459118 | 0.061542 | 0 | 0 | −0.17407 | 0 | 0 | 0.620493 |
| 1347 | −5.22869 | 1.40571 | 0.067264 | 0 | 0 | 0 | −0.0385 | −0.16673 | 0.675654 |
| 1348 | −5.16417 | 1.378578 | 0.066377 | 0 | 0 | 0 | −0.0387 | −0.17212 | 0.669749 |
| 1349 | −5.13028 | 1.396981 | 0.065962 | 0 | 0 | 0 | −0.04327 | −0.19475 | 0.69679 |
| 1350 | −5.42236 | 1.353027 | 0.071106 | 0 | 0 | 0 | −0.04582 | −0.18651 | 0.678428 |
| 1351 | −5.39615 | 1.360007 | 0.071036 | 0 | 0 | 0 | −0.04928 | −0.20915 | 0.712797 |
| 1352 | −5.41258 | 1.356925 | 0.070971 | 0 | 0 | 0 | −0.05087 | −0.20147 | 0.691179 |
| 1353 | −5.08923 | 1.453483 | 0.063822 | 0 | 0 | 0 | −0.04931 | −0.16021 | 0.579396 |
| 1354 | −5.02338 | 1.464752 | 0.063077 | 0 | 0 | 0 | −0.05274 | −0.18191 | 0.618019 |
| 1355 | −5.0278 | 1.454316 | 0.062938 | 0 | 0 | 0 | −0.05328 | −0.17725 | 0.59412 |
| 1356 | −5.24237 | 1.415479 | 0.067254 | 0 | 0 | 0 | −0.05531 | −0.19197 | 0.6277 |
| 1357 | −5.0407 | 1.350363 | 0.064105 | 0 | 0 | 0 | −0.04448 | 0 | 0.665078 |
| 1358 | −5.01963 | 1.370563 | 0.063699 | 0 | 0 | 0 | −0.04985 | 0 | 0.673141 |
| 1359 | −4.99984 | 1.356657 | 0.06331 | 0 | 0 | 0 | −0.0489 | 0 | 0.661128 |
| 1360 | −5.28743 | 1.333391 | 0.068328 | 0 | 0 | 0 | −0.05946 | 0 | 0.654453 |
| 1361 | −4.94227 | 1.43173 | 0.061213 | 0 | 0 | 0 | −0.05813 | 0 | 0.590732 |
| 1362 | −5.13302 | 1.365819 | 0.06633 | 0 | 0 | 0 | 0 | −0.17881 | 0.691438 |
| 1363 | −5.0942 | 1.378306 | 0.065924 | 0 | 0 | 0 | 0 | −0.2035 | 0.717646 |
| 1364 | −5.06896 | 1.366095 | 0.065407 | 0 | 0 | 0 | 0 | −0.19809 | 0.70411 |
| 1365 | −5.3565 | 1.338146 | 0.070748 | 0 | 0 | 0 | 0 | −0.21266 | 0.722015 |
| 1366 | −4.96683 | 1.43875 | 0.062595 | 0 | 0 | 0 | 0 | −0.18561 | 0.629107 |
| 1367 | −4.96405 | 1.343183 | 0.063183 | 0 | 0 | 0 | 0 | 0 | 0.679082 |
| 1368 | −0.93114 | 1.226068 | 0 | 0.118195 | 0.045044 | −0.07112 | −0.05985 | −0.10364 | 0.795137 |
| 1369 | −0.91891 | 1.087658 | 0 | 0.086527 | 0.053667 | −0.05469 | −0.0652 | −0.12637 | 0.691942 |
| 1370 | −0.91689 | 1.159035 | 0 | 0.086868 | 0.032337 | −0.06483 | −0.07209 | −0.12248 | 0.735669 |
| 1371 | −0.91795 | 1.140571 | 0 | 0.099187 | 0.039693 | −0.09654 | −0.06787 | −0.12617 | 0.73258 |
| 1372 | −0.90788 | 1.155091 | 0 | 0.091579 | 0.03271 | −0.06356 | −0.07512 | −0.15133 | 0.757487 |
| 1373 | −0.94057 | 1.147988 | 0 | 0.086697 | −0.00798 | −0.12 | −0.07161 | 0 | 0.74412 |
| 1374 | −0.88502 | 1.106134 | 0 | 0.118424 | 0.029174 | −0.09599 | −0.04595 | 0 | 0.808443 |
| 1375 | −0.90753 | 1.073335 | 0 | 0.074319 | 0.007759 | −0.08651 | −0.06734 | 0 | 0.721449 |
| 1376 | −0.92181 | 1.211497 | 0 | 0.105354 | 0.004385 | −0.09857 | −0.06114 | 0 | 0.814992 |
| 1377 | −0.91169 | 1.063037 | 0 | 0.086404 | 0.011799 | −0.11542 | −0.06405 | 0 | 0.721056 |
| 1378 | −0.92855 | 1.08528 | 0 | 0.075197 | 0.000962 | −0.08836 | −0.06934 | 0 | 0.716354 |
| 1379 | −0.91249 | 1.191525 | 0 | 0.124455 | 0.00738 | −0.14016 | −0.05362 | 0 | 0.824841 |
| 1380 | −0.92992 | 1.22043 | 0 | 0.111012 | −0.00723 | −0.10707 | −0.06283 | 0 | 0.82472 |
| 1381 | −0.91306 | 1.132727 | 0 | 0.087532 | −0.00549 | −0.12372 | −0.07012 | 0 | 0.757512 |
| 1382 | −0.91953 | 1.151958 | 0 | 0.076215 | −0.01992 | −0.10032 | −0.07583 | 0 | 0.764277 |
| 1383 | −0.8749 | 1.132962 | 0 | 0.088244 | 0.022272 | −0.095 | 0 | −0.15192 | 0.777683 |
| 1384 | −0.84931 | 1.100057 | 0 | 0.120173 | 0.052671 | −0.07474 | 0 | −0.11573 | 0.814095 |
| 1385 | −0.86114 | 1.066471 | 0 | 0.073712 | 0.03224 | −0.06441 | 0 | −0.13314 | 0.736808 |
| 1386 | −0.88924 | 1.208956 | 0 | 0.102424 | 0.02297 | −0.07775 | 0 | −0.11057 | 0.815933 |
| 1387 | −0.86103 | 1.052975 | 0 | 0.087361 | 0.041056 | −0.09376 | 0 | −0.13635 | 0.741367 |
| 1388 | −0.85637 | 1.067123 | 0 | 0.078386 | 0.033868 | −0.06207 | 0 | −0.15896 | 0.757099 |
| 1389 | −0.87582 | 1.186078 | 0 | 0.124239 | 0.030114 | −0.11844 | 0 | −0.11545 | 0.831286 |
| 1390 | −0.86965 | 1.207348 | 0 | 0.113657 | 0.020504 | −0.07918 | 0 | −0.14757 | 0.851074 |
| 1391 | −0.86679 | 1.125721 | 0 | 0.086091 | 0.019402 | −0.1013 | 0 | −0.13228 | 0.771958 |
| 1392 | −0.85024 | 1.137762 | 0 | 0.077251 | 0.008 | −0.07264 | 0 | −0.15854 | 0.800011 |
| 1393 | −0.85366 | 1.093318 | 0 | 0.104056 | 0.010272 | −0.10522 | 0 | 0 | 0.821191 |
| 1394 | −0.83971 | 1.072837 | 0 | 0.124625 | 0.012966 | −0.14537 | 0 | 0 | 0.838427 |
| 1395 | −0.861 | 1.101188 | 0 | 0.109149 | −0.00146 | −0.11228 | 0 | 0 | 0.829811 |
| 1396 | −0.86696 | 1.048009 | 0 | 0.072508 | −0.007 | −0.12267 | 0 | 0 | 0.752366 |
| 1397 | −0.87576 | 1.066729 | 0 | 0.060652 | −0.02134 | −0.10056 | 0 | 0 | 0.754075 |
| 1398 | −0.89472 | 1.06631 | 0 | 0.072487 | −0.00918 | −0.11804 | 0 | 0 | 0.741113 |
| 1399 | −0.88039 | 1.179975 | 0 | 0.108758 | −0.0113 | −0.14337 | 0 | 0 | 0.839459 |
| 1400 | −0.89147 | 1.205381 | 0 | 0.094181 | −0.02936 | −0.11549 | 0 | 0 | 0.8424 |
| 1401 | −0.89636 | 1.196026 | 0 | 0.112321 | −0.01763 | −0.14313 | 0 | 0 | 0.842747 |
| 1402 | −0.89205 | 1.134683 | 0 | 0.07179 | −0.03022 | −0.12673 | 0 | 0 | 0.779554 |
| 1403 | −0.89365 | 1.119805 | 0 | 0.107371 | 0.011212 | 0 | −0.0722 | −0.16018 | 0.763783 |
| 1404 | −0.86058 | 1.088097 | 0 | 0.133349 | 0.043418 | 0 | −0.04654 | −0.12284 | 0.806219 |
| 1405 | −0.87302 | 1.056335 | 0 | 0.090047 | 0.028164 | 0 | −0.06694 | −0.13818 | 0.731029 |
| 1406 | −0.87972 | 1.041938 | 0 | 0.104779 | 0.029097 | 0 | −0.06363 | −0.14363 | 0.726061 |
| 1407 | −0.90265 | 1.195834 | 0 | 0.118573 | 0.015991 | 0 | −0.06238 | −0.11844 | 0.8078 |
| 1408 | −0.86731 | 1.05597 | 0 | 0.095161 | 0.029232 | 0 | −0.06908 | −0.16399 | 0.75223 |
| 1409 | −0.89774 | 1.169632 | 0 | 0.140641 | 0.013457 | 0 | −0.05566 | −0.12746 | 0.809903 |
| 1410 | −0.88223 | 1.192843 | 0 | 0.130252 | 0.012465 | 0 | −0.06426 | −0.15599 | 0.844259 |
| 1411 | −0.88749 | 1.113823 | 0 | 0.105273 | 0.008117 | 0 | −0.07093 | −0.14122 | 0.756618 |
| 1412 | −0.86298 | 1.126293 | 0 | 0.096134 | 0.003011 | 0 | −0.07708 | −0.16547 | 0.79586 |
| 1413 | −0.87074 | 1.078689 | 0 | 0.119366 | −0.00731 | 0 | −0.05009 | 0 | 0.810018 |
| 1414 | −0.86684 | 1.056627 | 0 | 0.139877 | −0.01592 | 0 | −0.04424 | 0 | 0.814309 |
| 1415 | −0.87855 | 1.085382 | 0 | 0.125035 | −0.02129 | 0 | −0.05113 | 0 | 0.817798 |
| 1416 | −0.89275 | 1.035075 | 0 | 0.091911 | −0.02656 | 0 | −0.06755 | 0 | 0.73285 |
| 1417 | −0.89422 | 1.052914 | 0 | 0.079978 | −0.03523 | 0 | −0.07169 | 0 | 0.743589 |
| 1418 | −0.919 | 1.052172 | 0 | 0.091815 | −0.02845 | 0 | −0.06818 | 0 | 0.721875 |
| 1419 | −0.9097 | 1.161656 | 0 | 0.126587 | −0.03611 | 0 | −0.05874 | 0 | 0.814349 |
| 1420 | −0.91185 | 1.187931 | 0 | 0.112783 | −0.04695 | 0 | −0.06583 | 0 | 0.829533 |
| 1421 | −0.92447 | 1.176159 | 0 | 0.130022 | −0.0424 | 0 | −0.05977 | 0 | 0.817542 |
| 1422 | −0.91933 | 1.119426 | 0 | 0.092617 | −0.0494 | 0 | −0.07459 | 0 | 0.759217 |
| 1423 | −0.82879 | 1.075191 | 0 | 0.119551 | 0.022487 | 0 | 0 | −0.12964 | 0.819658 |
| 1424 | −0.81768 | 1.050041 | 0 | 0.143474 | 0.020475 | 0 | 0 | −0.13922 | 0.832178 |
| 1425 | −0.81028 | 1.070264 | 0 | 0.130787 | 0.020277 | 0 | 0 | −0.16528 | 0.85064 |
| 1426 | −0.83388 | 1.026811 | 0 | 0.091762 | 0.008965 | 0 | 0 | −0.15049 | 0.759424 |
| 1427 | −0.81431 | 1.036711 | 0 | 0.081818 | 0.003635 | 0 | 0 | −0.17267 | 0.78925 |
| 1428 | −0.84173 | 1.035638 | 0 | 0.094487 | 0.013023 | 0 | 0 | −0.16728 | 0.767196 |
| 1429 | −0.86386 | 1.159227 | 0 | 0.125337 | −0.00524 | 0 | 0 | −0.13361 | 0.827962 |
| 1430 | −0.84386 | 1.178638 | 0 | 0.11369 | −0.01184 | 0 | 0 | −0.16247 | 0.861641 |
| 1431 | −0.85769 | 1.165558 | 0 | 0.133018 | −0.00519 | 0 | 0 | −0.15951 | 0.851119 |
| 1432 | −0.84332 | 1.106749 | 0 | 0.093305 | −0.01206 | 0 | 0 | −0.16818 | 0.801487 |
| 1433 | −0.84298 | 1.04896 | 0 | 0.125627 | −0.03206 | 0 | 0 | 0 | 0.82362 |
| 1434 | −0.8492 | 1.073932 | 0 | 0.110263 | −0.04241 | 0 | 0 | 0 | 0.829544 |
| 1435 | −0.85556 | 1.061532 | 0 | 0.128933 | −0.03912 | 0 | 0 | 0 | 0.827065 |
| 1436 | −0.87552 | 1.03844 | 0 | 0.076812 | −0.05101 | 0 | 0 | 0 | 0.752224 |
| 1437 | −0.89231 | 1.166367 | 0 | 0.113359 | −0.06354 | 0 | 0 | 0 | 0.833862 |
| 1438 | −0.88747 | 1.134432 | 0 | 0.103365 | 0 | −0.08872 | −0.06926 | −0.14764 | 0.77553 |
| 1439 | −0.86738 | 1.109301 | 0 | 0.132772 | 0 | −0.06301 | −0.04043 | −0.10893 | 0.807443 |
| 1440 | −0.87604 | 1.071193 | 0 | 0.088964 | 0 | −0.05593 | −0.06264 | −0.12786 | 0.733487 |
| 1441 | −0.87765 | 1.05954 | 0 | 0.102655 | 0 | −0.08407 | −0.05915 | −0.12965 | 0.73602 |
| 1442 | −0.90212 | 1.210867 | 0 | 0.115676 | 0 | −0.07095 | −0.05858 | −0.10689 | 0.812594 |
| 1443 | −0.87175 | 1.071662 | 0 | 0.09421 | 0 | −0.05331 | −0.06524 | −0.15318 | 0.753694 |
| 1444 | −0.88931 | 1.189408 | 0 | 0.13638 | 0 | −0.11076 | −0.05106 | −0.11089 | 0.826386 |
| 1445 | −0.88184 | 1.208065 | 0 | 0.126893 | 0 | −0.07284 | −0.06096 | −0.14409 | 0.848415 |
| 1446 | −0.87863 | 1.127384 | 0 | 0.100613 | 0 | −0.09532 | −0.06769 | −0.12843 | 0.77 |
| 1447 | −0.85983 | 1.136651 | 0 | 0.092027 | 0 | −0.06829 | −0.07485 | −0.15615 | 0.800181 |
| 1448 | −0.86293 | 1.093246 | 0 | 0.113601 | 0 | −0.09993 | −0.04602 | 0 | 0.816478 |
| 1449 | −0.84829 | 1.073586 | 0 | 0.132499 | 0 | −0.13999 | −0.03905 | 0 | 0.833551 |
| 1450 | −0.86819 | 1.098161 | 0 | 0.11722 | 0 | −0.10949 | −0.0479 | 0 | 0.825301 |
| 1451 | −0.87377 | 1.043501 | 0 | 0.083514 | 0 | −0.12105 | −0.06583 | 0 | 0.748727 |
| 1452 | −0.88088 | 1.058788 | 0 | 0.071157 | 0 | −0.10175 | −0.0714 | 0 | 0.750387 |
| 1453 | −0.90149 | 1.060823 | 0 | 0.083112 | 0 | −0.11703 | −0.06682 | 0 | 0.736749 |
| 1454 | −0.88678 | 1.174309 | 0 | 0.11744 | 0 | −0.14281 | −0.05635 | 0 | 0.834269 |
| 1455 | −0.89595 | 1.19554 | 0 | 0.102791 | 0 | −0.11845 | −0.06551 | 0 | 0.837293 |
| 1456 | −0.90169 | 1.188263 | 0 | 0.120256 | 0 | −0.14393 | −0.0579 | 0 | 0.83729 |
| 1457 | −0.89633 | 1.124555 | 0 | 0.081662 | 0 | −0.12998 | −0.07495 | 0 | 0.775012 |
| 1458 | −0.83053 | 1.09176 | 0 | 0.117811 | 0 | −0.07384 | 0 | −0.11694 | 0.823386 |
| 1459 | −0.81269 | 1.070371 | 0 | 0.140423 | 0 | −0.11183 | 0 | −0.12149 | 0.844956 |
| 1460 | −0.81259 | 1.08734 | 0 | 0.128431 | 0 | −0.07458 | 0 | −0.15204 | 0.853536 |
| 1461 | −0.82678 | 1.03922 | 0 | 0.087938 | 0 | −0.09308 | 0 | −0.13816 | 0.77057 |
| 1462 | −0.81188 | 1.046689 | 0 | 0.078254 | 0 | −0.06797 | 0 | −0.16358 | 0.793097 |
| 1463 | −0.8377 | 1.049455 | 0 | 0.091282 | 0 | −0.08556 | 0 | −0.155 | 0.776208 |
| 1464 | −0.85217 | 1.174027 | 0 | 0.119599 | 0 | −0.11557 | 0 | −0.1187 | 0.843059 |
| 1465 | −0.83826 | 1.188668 | 0 | 0.108061 | 0 | −0.0835 | 0 | −0.15217 | 0.86598 |
| 1466 | −0.84807 | 1.180797 | 0 | 0.127495 | 0 | −0.11094 | 0 | −0.14505 | 0.864126 |
| 1467 | −0.83237 | 1.115714 | 0 | 0.086927 | 0 | −0.09733 | 0 | −0.15754 | 0.812218 |
| 1468 | −0.82117 | 1.061738 | 0 | 0.117461 | 0 | −0.14736 | 0 | 0 | 0.84307 |
| 1469 | −0.83347 | 1.082283 | 0 | 0.100784 | 0 | −0.12321 | 0 | 0 | 0.838676 |
| 1470 | −0.83443 | 1.073595 | 0 | 0.119568 | 0 | −0.14727 | 0 | 0 | 0.84525 |
| 1471 | −0.85304 | 1.041978 | 0 | 0.065677 | 0 | −0.12921 | 0 | 0 | 0.766817 |
| 1472 | −0.86607 | 1.173654 | 0 | 0.101452 | 0 | −0.15188 | 0 | 0 | 0.851255 |
| 1473 | −0.84062 | 1.078974 | 0 | 0.12997 | 0 | 0 | −0.04537 | −0.12503 | 0.815343 |
| 1474 | −0.82803 | 1.053374 | 0 | 0.152069 | 0 | 0 | −0.03971 | −0.13479 | 0.827721 |
| 1475 | −0.82184 | 1.073424 | 0 | 0.140658 | 0 | 0 | −0.04644 | −0.16066 | 0.846422 |
| 1476 | −0.84345 | 1.026926 | 0 | 0.104368 | 0 | 0 | −0.06434 | −0.147 | 0.75673 |
| 1477 | −0.82323 | 1.035416 | 0 | 0.094907 | 0 | 0 | −0.06933 | −0.1699 | 0.78747 |
| 1478 | −0.85234 | 1.036179 | 0 | 0.107409 | 0 | 0 | −0.06474 | −0.16317 | 0.763819 |
| 1479 | −0.87145 | 1.155699 | 0 | 0.135154 | 0 | 0 | −0.05803 | −0.13275 | 0.82365 |
| 1480 | −0.85069 | 1.173394 | 0 | 0.124679 | 0 | 0 | −0.06593 | −0.1624 | 0.858694 |
| 1481 | −0.865 | 1.161166 | 0 | 0.142857 | 0 | 0 | −0.05894 | −0.1587 | 0.846975 |
| 1482 | −0.84976 | 1.101459 | 0 | 0.10571 | 0 | 0 | −0.07479 | −0.1679 | 0.799554 |
| 1483 | −0.84566 | 1.039124 | 0 | 0.130479 | 0 | 0 | −0.04901 | 0 | 0.817853 |
| 1484 | −0.85123 | 1.061226 | 0 | 0.11571 | 0 | 0 | −0.05591 | 0 | 0.823159 |
| 1485 | −0.85748 | 1.049663 | 0 | 0.13279 | 0 | 0 | −0.0499 | 0 | 0.820397 |
| 1486 | −0.87786 | 1.023487 | 0 | 0.084167 | 0 | 0 | −0.07393 | 0 | 0.744864 |
| 1487 | −0.89399 | 1.148103 | 0 | 0.118121 | 0 | 0 | −0.06668 | 0 | 0.823498 |
| 1488 | −0.80004 | 1.041698 | 0 | 0.137296 | 0 | 0 | 0 | −0.14211 | 0.838219 |
| 1489 | −0.78627 | 1.056257 | 0 | 0.124845 | 0 | 0 | 0 | −0.16982 | 0.859638 |
| 1490 | −0.79387 | 1.045133 | 0 | 0.144729 | 0 | 0 | 0 | −0.16665 | 0.858513 |
| 1491 | −0.80153 | 1.015973 | 0 | 0.091411 | 0 | 0 | 0 | −0.17459 | 0.795933 |
| 1492 | −0.82765 | 1.147162 | 0 | 0.124557 | 0 | 0 | 0 | −0.16766 | 0.863065 |
| 1493 | −0.82999 | 1.038216 | 0 | 0.115481 | 0 | 0 | 0 | 0 | 0.82921 |
| 1494 | −0.8981 | 1.142031 | 0 | 0 | 0.041926 | −0.10159 | −0.05997 | −0.14675 | 0.759941 |
| 1495 | −0.87672 | 1.114856 | 0 | 0 | 0.071665 | −0.08331 | −0.03202 | −0.11043 | 0.788806 |
| 1496 | −0.88018 | 1.074662 | 0 | 0 | 0.05059 | −0.06994 | −0.0558 | −0.12882 | 0.724416 |
| 1497 | −0.88357 | 1.063706 | 0 | 0 | 0.059518 | −0.10006 | −0.05165 | −0.13149 | 0.723098 |
| 1498 | −0.91529 | 1.218911 | 0 | 0 | 0.043365 | −0.08481 | −0.04827 | −0.10647 | 0.794462 |
| 1499 | −0.87637 | 1.075624 | 0 | 0 | 0.052162 | −0.0684 | −0.058 | −0.15376 | 0.743357 |
| 1500 | −0.90709 | 1.199467 | 0 | 0 | 0.050829 | −0.1258 | −0.04009 | −0.11057 | 0.800475 |
| 1501 | −0.89824 | 1.218766 | 0 | 0 | 0.0414 | −0.08716 | −0.0498 | −0.14156 | 0.826816 |
| 1502 | −0.8894 | 1.135108 | 0 | 0 | 0.039025 | −0.1074 | −0.05812 | −0.12789 | 0.755404 |
| 1503 | −0.86996 | 1.145277 | 0 | 0 | 0.027676 | −0.07866 | −0.06521 | −0.15381 | 0.788835 |
| 1504 | −0.87642 | 1.105738 | 0 | 0 | 0.029554 | −0.11125 | −0.03516 | 0 | 0.801075 |
| 1505 | −0.86629 | 1.089729 | 0 | 0 | 0.032839 | −0.15178 | −0.028 | 0 | 0.811922 |
| 1506 | −0.88394 | 1.11434 | 0 | 0 | 0.017863 | −0.11879 | −0.03657 | 0 | 0.809131 |
| 1507 | −0.88482 | 1.055946 | 0 | 0 | 0.011635 | −0.12697 | −0.05599 | 0 | 0.739471 |
| 1508 | −0.89074 | 1.071856 | 0 | 0 | −0.00326 | −0.10439 | −0.06157 | 0 | 0.745372 |
| 1509 | −0.91251 | 1.073652 | 0 | 0 | 0.009348 | −0.12257 | −0.05743 | 0 | 0.727537 |
| 1510 | −0.90655 | 1.191907 | 0 | 0 | 0.009476 | −0.14878 | −0.04305 | 0 | 0.81532 |
| 1511 | −0.91386 | 1.214327 | 0 | 0 | −0.00878 | −0.12071 | −0.05185 | 0 | 0.824927 |
| 1512 | −0.92286 | 1.207996 | 0 | 0 | 0.003709 | −0.14866 | −0.04477 | 0 | 0.817534 |
| 1513 | −0.9101 | 1.141179 | 0 | 0 | −0.0107 | −0.13107 | −0.06317 | 0 | 0.767566 |
| 1514 | −0.84227 | 1.098891 | 0 | 0 | 0.053496 | −0.08936 | 0 | −0.11637 | 0.806342 |
| 1515 | −0.829 | 1.079929 | 0 | 0 | 0.061757 | −0.12911 | 0 | −0.12102 | 0.820485 |
| 1516 | −0.82674 | 1.096679 | 0 | 0 | 0.05223 | −0.0911 | 0 | −0.14989 | 0.833835 |
| 1517 | −0.83518 | 1.045398 | 0 | 0 | 0.04023 | −0.10452 | 0 | −0.13774 | 0.759214 |
| 1518 | −0.81978 | 1.053507 | 0 | 0 | 0.028518 | −0.07745 | 0 | −0.16171 | 0.784455 |
| 1519 | −0.84588 | 1.055203 | 0 | 0 | 0.043851 | −0.09819 | 0 | −0.15458 | 0.763767 |
| 1520 | −0.87114 | 1.185285 | 0 | 0 | 0.032805 | −0.12645 | 0 | −0.11637 | 0.821379 |
| 1521 | −0.85624 | 1.201194 | 0 | 0 | 0.018755 | −0.0923 | 0 | −0.14764 | 0.849425 |
| 1522 | −0.86919 | 1.193397 | 0 | 0 | 0.033145 | −0.1223 | 0 | −0.1413 | 0.839445 |
| 1523 | −0.84541 | 1.125882 | 0 | 0 | 0.018989 | −0.10508 | 0 | −0.15429 | 0.800152 |
| 1524 | −0.8406 | 1.078273 | 0 | 0 | 0.018506 | −0.15463 | 0 | 0 | 0.823994 |
| 1525 | −0.85125 | 1.099484 | 0 | 0 | −0.00025 | −0.12667 | 0 | 0 | 0.825378 |
| 1526 | −0.85488 | 1.092193 | 0 | 0 | 0.01137 | −0.15336 | 0 | 0 | 0.825822 |
| 1527 | −0.86552 | 1.056488 | 0 | 0 | −0.01222 | −0.12896 | 0 | 0 | 0.761363 |
| 1528 | −0.88802 | 1.194289 | 0 | 0 | −0.01633 | −0.15113 | 0 | 0 | 0.83703 |
| 1529 | −0.85558 | 1.088783 | 0 | 0 | 0.041721 | 0 | −0.03395 | −0.12485 | 0.796741 |
| 1530 | −0.85024 | 1.068337 | 0 | 0 | 0.040281 | 0 | −0.02719 | −0.13341 | 0.800395 |
| 1531 | −0.83951 | 1.085973 | 0 | 0 | 0.03921 | 0 | −0.03475 | −0.15868 | 0.8251 |
| 1532 | −0.85684 | 1.037678 | 0 | 0 | 0.028057 | 0 | −0.05329 | −0.14574 | 0.742594 |
| 1533 | −0.83428 | 1.045297 | 0 | 0 | 0.022393 | 0 | −0.05893 | −0.16772 | 0.77705 |
| 1534 | −0.86562 | 1.046546 | 0 | 0 | 0.031764 | 0 | −0.05425 | −0.16183 | 0.748446 |
| 1535 | −0.8949 | 1.171656 | 0 | 0 | 0.015856 | 0 | −0.04291 | −0.12896 | 0.799486 |
| 1536 | −0.87158 | 1.18928 | 0 | 0 | 0.008991 | 0 | −0.05106 | −0.15707 | 0.840205 |
| 1537 | −0.89085 | 1.178967 | 0 | 0 | 0.016138 | 0 | −0.04397 | −0.15332 | 0.8198 |
| 1538 | −0.86724 | 1.116253 | 0 | 0 | 0.00797 | 0 | −0.06142 | −0.16316 | 0.784854 |
| 1539 | −0.87011 | 1.064778 | 0 | 0 | −0.01139 | 0 | −0.03206 | 0 | 0.798237 |
| 1540 | −0.87239 | 1.086185 | 0 | 0 | −0.02258 | 0 | −0.03907 | 0 | 0.809962 |
| 1541 | −0.88275 | 1.07767 | 0 | 0 | −0.01842 | 0 | −0.03335 | 0 | 0.800871 |
| 1542 | −0.89379 | 1.046034 | 0 | 0 | −0.03161 | 0 | −0.05921 | 0 | 0.73982 |
| 1543 | −0.91881 | 1.177513 | 0 | 0 | −0.0416 | 0 | −0.04751 | 0 | 0.810434 |
| 1544 | −0.82389 | 1.057726 | 0 | 0 | 0.02579 | 0 | 0 | −0.13844 | 0.813927 |
| 1545 | −0.80682 | 1.071249 | 0 | 0 | 0.01928 | 0 | 0 | −0.16503 | 0.84098 |
| 1546 | −0.81914 | 1.06247 | 0 | 0 | 0.026181 | 0 | 0 | −0.16179 | 0.832062 |
| 1547 | −0.81706 | 1.0292 | 0 | 0 | 0.009563 | 0 | 0 | −0.1703 | 0.784109 |
| 1548 | −0.8544 | 1.166663 | 0 | 0 | −0.00361 | 0 | 0 | −0.1598 | 0.842122 |
| 1549 | −0.8567 | 1.067167 | 0 | 0 | −0.03505 | 0 | 0 | 0 | 0.814116 |
| 1550 | −0.85976 | 1.110934 | 0 | 0 | 0 | −0.07885 | −0.02736 | −0.10938 | 0.801372 |
| 1551 | −0.84723 | 1.094494 | 0 | 0 | 0 | −0.11726 | −0.02042 | −0.11265 | 0.815111 |
| 1552 | −0.84433 | 1.1089 | 0 | 0 | 0 | −0.08063 | −0.02901 | −0.14236 | 0.829115 |
| 1553 | −0.85169 | 1.054583 | 0 | 0 | 0 | −0.09604 | −0.04868 | −0.13083 | 0.755216 |
| 1554 | −0.83413 | 1.059766 | 0 | 0 | 0 | −0.07071 | −0.05521 | −0.15611 | 0.782188 |
| 1555 | −0.86382 | 1.06525 | 0 | 0 | 0 | −0.08919 | −0.05009 | −0.14681 | 0.758939 |
| 1556 | −0.88481 | 1.19188 | 0 | 0 | 0 | −0.11946 | −0.03864 | −0.1112 | 0.817556 |
| 1557 | −0.86805 | 1.204486 | 0 | 0 | 0 | −0.08742 | −0.0482 | −0.14394 | 0.846912 |
| 1558 | −0.88321 | 1.200019 | 0 | 0 | 0 | −0.11539 | −0.04032 | −0.13588 | 0.835726 |
| 1559 | −0.85775 | 1.129682 | 0 | 0 | 0 | −0.10034 | −0.05903 | −0.14991 | 0.798455 |
| 1560 | −0.84954 | 1.082351 | 0 | 0 | 0 | −0.1491 | −0.0276 | 0 | 0.820027 |
| 1561 | −0.858 | 1.098765 | 0 | 0 | 0 | −0.12471 | −0.0368 | 0 | 0.821361 |
| 1562 | −0.86261 | 1.094382 | 0 | 0 | 0 | −0.14938 | −0.02973 | 0 | 0.822037 |
| 1563 | −0.87205 | 1.052614 | 0 | 0 | 0 | −0.12932 | −0.0593 | 0 | 0.757247 |
| 1564 | −0.89345 | 1.189136 | 0 | 0 | 0 | −0.15257 | −0.04724 | 0 | 0.83163 |
| 1565 | −0.81802 | 1.079382 | 0 | 0 | 0 | −0.12115 | 0 | −0.11906 | 0.828804 |
| 1566 | −0.80649 | 1.088975 | 0 | 0 | 0 | −0.09004 | 0 | −0.15018 | 0.84699 |
| 1567 | −0.8158 | 1.085135 | 0 | 0 | 0 | −0.11609 | 0 | −0.14258 | 0.844647 |
| 1568 | −0.80942 | 1.041556 | 0 | 0 | 0 | −0.09736 | 0 | −0.15744 | 0.795675 |
| 1569 | −0.84293 | 1.181955 | 0 | 0 | 0 | −0.11926 | 0 | −0.14428 | 0.856526 |
| 1570 | −0.83283 | 1.079246 | 0 | 0 | 0 | −0.1561 | 0 | 0 | 0.835239 |
| 1571 | −0.8342 | 1.064214 | 0 | 0 | 0 | 0 | −0.02602 | −0.13349 | 0.810696 |
| 1572 | −0.81716 | 1.076178 | 0 | 0 | 0 | 0 | −0.03297 | −0.16067 | 0.837785 |
| 1573 | −0.82979 | 1.069218 | 0 | 0 | 0 | 0 | −0.02689 | −0.15654 | 0.828817 |
| 1574 | −0.82712 | 1.031404 | 0 | 0 | 0 | 0 | −0.0543 | −0.16643 | 0.781361 |
| 1575 | −0.86171 | 1.16511 | 0 | 0 | 0 | 0 | −0.04549 | −0.15863 | 0.838133 |
| 1576 | −0.85822 | 1.0577 | 0 | 0 | 0 | 0 | −0.03791 | 0 | 0.808047 |
| 1577 | −0.79887 | 1.054139 | 0 | 0 | 0 | 0 | 0 | −0.16466 | 0.843206 |
| 1578 | −0.15564 | 0 | 0 | 0 | 0 | −0.09044 | −0.02414 | −0.12328 | 0.911882 |
| 1579 | −0.14235 | 0 | 0 | 0 | 0 | −0.05505 | −0.0315 | −0.16155 | 0.925522 |
| 1580 | −0.1533 | 0 | 0 | 0 | 0 | −0.08308 | −0.02568 | −0.15275 | 0.923611 |
| 1581 | −0.14787 | 0 | 0 | 0 | 0 | −0.0742 | −0.05315 | −0.1672 | 0.921548 |
| 1582 | −0.1472 | 0 | 0 | 0 | 0 | −0.08272 | −0.04614 | −0.15375 | 0.916383 |
| 1583 | −0.17527 | 0 | 0 | 0 | 0 | −0.12236 | −0.0339 | 0 | 0.913722 |
| 1584 | −0.12813 | 0 | 0 | 0 | 0 | −0.08995 | 0 | −0.16033 | 0.94 |
| 1585 | −0.14815 | 0 | 0 | 0 | 0 | 0 | −0.02908 | −0.16983 | 0.925187 |
| 1586 | −3.65788 | 0 | 0.054165 | 0.244601 | 0.090637 | −0.0769 | −0.09282 | 0 | 0.630358 |
| 1587 | −4.06448 | 0 | 0.062232 | 0.239224 | 0.087914 | −0.07629 | −0.08867 | 0 | 0.786788 |
| 1588 | −3.83356 | 0 | 0.057684 | 0.221666 | 0.073929 | −0.08159 | −0.09194 | 0 | 0.678801 |
| 1589 | −3.8716 | 0 | 0.0584 | 0.240274 | 0.080008 | −0.11294 | −0.09342 | 0 | 0.703487 |
| 1590 | −3.86397 | 0 | 0.058069 | 0.228216 | 0.066412 | −0.08285 | −0.09851 | 0 | 0.678623 |
| 1591 | −3.69119 | 0 | 0.055321 | 0.237983 | 0.108491 | −0.04709 | 0 | −0.15241 | 0.621779 |
| 1592 | −4.11127 | 0 | 0.063954 | 0.242195 | 0.113057 | −0.04261 | 0 | −0.18287 | 0.812416 |
| 1593 | −3.86031 | 0 | 0.058914 | 0.219039 | 0.093235 | −0.04973 | 0 | −0.16736 | 0.68712 |
| 1594 | −3.88682 | 0 | 0.059534 | 0.23801 | 0.10604 | −0.08157 | 0 | −0.17378 | 0.720743 |
| 1595 | −3.8622 | 0 | 0.059298 | 0.231327 | 0.095652 | −0.04542 | 0 | −0.20527 | 0.727721 |
| 1596 | −3.80004 | 0 | 0.057697 | 0.218646 | 0.051701 | −0.11524 | 0 | 0 | 0.727825 |
| 1597 | −3.67425 | 0 | 0.056066 | 0.252748 | 0.08556 | −0.09233 | 0 | 0 | 0.796527 |
| 1598 | −3.55094 | 0 | 0.053188 | 0.220132 | 0.065238 | −0.08687 | 0 | 0 | 0.678359 |
| 1599 | −3.97903 | 0 | 0.061473 | 0.215078 | 0.061213 | −0.09059 | 0 | 0 | 0.81232 |
| 1600 | −4.00833 | 0 | 0.061928 | 0.232785 | 0.068612 | −0.12304 | 0 | 0 | 0.827394 |
| 1601 | −3.98912 | 0 | 0.06147 | 0.218884 | 0.053184 | −0.09046 | 0 | 0 | 0.806257 |
| 1602 | −3.51904 | 0 | 0.0529 | 0.243664 | 0.071457 | −0.12366 | 0 | 0 | 0.715828 |
| 1603 | −3.5116 | 0 | 0.052525 | 0.227636 | 0.054591 | −0.09102 | 0 | 0 | 0.682399 |
| 1604 | −3.76041 | 0 | 0.057278 | 0.215155 | 0.055596 | −0.12102 | 0 | 0 | 0.741048 |
| 1605 | −3.74882 | 0 | 0.056987 | 0.202707 | 0.038799 | −0.09512 | 0 | 0 | 0.723079 |
| 1606 | −3.73596 | 0 | 0.055701 | 0.258171 | 0.113428 | 0 | −0.09216 | −0.15714 | 0.613871 |
| 1607 | −4.14609 | 0 | 0.064199 | 0.260385 | 0.117551 | 0 | −0.08604 | −0.18568 | 0.805151 |
| 1608 | −3.89345 | 0 | 0.059105 | 0.237944 | 0.09726 | 0 | −0.08944 | −0.17133 | 0.678724 |
| 1609 | −3.94015 | 0 | 0.059914 | 0.259661 | 0.103254 | 0 | −0.09237 | −0.18142 | 0.702356 |
| 1610 | −3.90142 | 0 | 0.059597 | 0.253037 | 0.100932 | 0 | −0.09741 | −0.20957 | 0.722685 |
| 1611 | −3.84768 | 0 | 0.057832 | 0.241364 | 0.038579 | 0 | −0.09588 | 0 | 0.703128 |
| 1612 | −3.70818 | 0 | 0.056175 | 0.270056 | 0.076158 | 0 | −0.07802 | 0 | 0.784719 |
| 1613 | −3.58551 | 0 | 0.05329 | 0.239328 | 0.058942 | 0 | −0.0884 | 0 | 0.665664 |
| 1614 | −4.00085 | 0 | 0.061385 | 0.233543 | 0.053643 | 0 | −0.08461 | 0 | 0.800955 |
| 1615 | −4.04397 | 0 | 0.061891 | 0.252611 | 0.051582 | 0 | −0.08517 | 0 | 0.803043 |
| 1616 | −4.02054 | 0 | 0.061503 | 0.240105 | 0.045099 | 0 | −0.09078 | 0 | 0.794082 |
| 1617 | −3.57763 | 0 | 0.053198 | 0.263478 | 0.0556 | 0 | −0.08907 | 0 | 0.688102 |
| 1618 | −3.55352 | 0 | 0.052722 | 0.249492 | 0.047638 | 0 | −0.0936 | 0 | 0.669907 |
| 1619 | −3.80181 | 0 | 0.057316 | 0.235558 | 0.040962 | 0 | −0.0895 | 0 | 0.715146 |
| 1620 | −3.77941 | 0 | 0.056983 | 0.223981 | 0.030986 | 0 | −0.09348 | 0 | 0.709188 |
| 1621 | −3.8524 | 0 | 0.059292 | 0.244368 | 0.081048 | 0 | 0 | −0.21357 | 0.758579 |
| 1622 | −3.76342 | 0 | 0.057961 | 0.272171 | 0.106989 | 0 | 0 | −0.1767 | 0.79996 |
| 1623 | −3.62587 | 0 | 0.054683 | 0.233877 | 0.084627 | 0 | 0 | −0.16166 | 0.664225 |
| 1624 | −4.06268 | 0 | 0.063493 | 0.237334 | 0.08572 | 0 | 0 | −0.19431 | 0.833125 |
| 1625 | −4.09945 | 0 | 0.063993 | 0.258144 | 0.093452 | 0 | 0 | −0.20304 | 0.845189 |
| 1626 | −4.07317 | 0 | 0.063887 | 0.250605 | 0.090234 | 0 | 0 | −0.23337 | 0.862819 |
| 1627 | −3.60204 | 0 | 0.054453 | 0.258953 | 0.088442 | 0 | 0 | −0.17076 | 0.697706 |
| 1628 | −3.55052 | 0 | 0.053855 | 0.249999 | 0.084064 | 0 | 0 | −0.20199 | 0.706864 |
| 1629 | −3.82656 | 0 | 0.058763 | 0.234589 | 0.075213 | 0 | 0 | −0.18665 | 0.741068 |
| 1630 | −3.7902 | 0 | 0.058555 | 0.227216 | 0.067521 | 0 | 0 | −0.21674 | 0.763836 |
| 1631 | −3.65459 | 0 | 0.055777 | 0.246935 | 0.049015 | 0 | 0 | 0 | 0.803801 |
| 1632 | −3.65179 | 0 | 0.055643 | 0.270988 | 0.044831 | 0 | 0 | 0 | 0.812548 |
| 1633 | −3.61122 | 0 | 0.054958 | 0.255053 | 0.036039 | 0 | 0 | 0 | 0.798956 |
| 1634 | −3.96256 | 0 | 0.061103 | 0.228298 | 0.026472 | 0 | 0 | 0 | 0.824336 |
| 1635 | −3.94012 | 0 | 0.060775 | 0.21529 | 0.014634 | 0 | 0 | 0 | 0.815626 |
| 1636 | −3.98402 | 0 | 0.061243 | 0.232218 | 0.02337 | 0 | 0 | 0 | 0.81477 |
| 1637 | −3.49486 | 0 | 0.052403 | 0.237562 | 0.031218 | 0 | 0 | 0 | 0.716439 |
| 1638 | −3.46515 | 0 | 0.05192 | 0.223538 | 0.019401 | 0 | 0 | 0 | 0.704606 |
| 1639 | −3.48205 | 0 | 0.052055 | 0.24349 | 0.025023 | 0 | 0 | 0 | 0.709064 |
| 1640 | −3.74102 | 0 | 0.056756 | 0.21506 | 0.010939 | 0 | 0 | 0 | 0.737087 |
| 1641 | −3.73 | 0 | 0.0556 | 0.262352 | 0 | −0.02623 | −0.08044 | −0.13986 | 0.617608 |
| 1642 | −4.14689 | 0 | 0.064164 | 0.265641 | 0 | −0.02064 | −0.07478 | −0.16966 | 0.806038 |
| 1643 | −3.88788 | 0 | 0.059058 | 0.241755 | 0 | −0.03135 | −0.0793 | −0.15603 | 0.68685 |
| 1644 | −3.92066 | 0 | 0.059731 | 0.262353 | 0 | −0.06044 | −0.08142 | −0.16007 | 0.715742 |
| 1645 | −3.89355 | 0 | 0.059492 | 0.256991 | 0 | −0.02621 | −0.08735 | −0.19311 | 0.728951 |
| 1646 | −3.83024 | 0 | 0.057925 | 0.241449 | 0 | −0.10053 | −0.08913 | 0 | 0.725951 |
| 1647 | −3.71256 | 0 | 0.056394 | 0.271869 | 0 | −0.07036 | −0.06721 | 0 | 0.788864 |
| 1648 | −3.5824 | 0 | 0.053429 | 0.241147 | 0 | −0.07016 | −0.08003 | 0 | 0.674728 |
| 1649 | −4.00464 | 0 | 0.061623 | 0.234965 | 0 | −0.074 | −0.07643 | 0 | 0.808597 |
| 1650 | −4.03769 | 0 | 0.062124 | 0.253409 | 0 | −0.10454 | −0.07676 | 0 | 0.821098 |
| 1651 | −4.01873 | 0 | 0.061681 | 0.240482 | 0 | −0.07486 | −0.08377 | 0 | 0.80347 |
| 1652 | −3.55498 | 0 | 0.053202 | 0.265178 | 0 | −0.10473 | −0.08002 | 0 | 0.710014 |
| 1653 | −3.54192 | 0 | 0.052767 | 0.250197 | 0 | −0.07591 | −0.0863 | 0 | 0.681566 |
| 1654 | −3.78707 | 0 | 0.057451 | 0.235837 | 0 | −0.10567 | −0.08198 | 0 | 0.737393 |
| 1655 | −3.77126 | 0 | 0.057114 | 0.223578 | 0 | −0.08334 | −0.08772 | 0 | 0.723043 |
| 1656 | −3.83921 | 0 | 0.059199 | 0.247943 | 0 | −0.06169 | 0 | −0.19432 | 0.771019 |
| 1657 | −3.77033 | 0 | 0.05803 | 0.277961 | 0 | −0.03613 | 0 | −0.1596 | 0.800664 |
| 1658 | −3.62295 | 0 | 0.054671 | 0.238643 | 0 | −0.0389 | 0 | −0.1462 | 0.671885 |
| 1659 | −4.06681 | 0 | 0.063555 | 0.242261 | 0 | −0.03695 | 0 | −0.17999 | 0.837473 |
| 1660 | −4.09265 | 0 | 0.063969 | 0.262638 | 0 | −0.06819 | 0 | −0.18267 | 0.854392 |
| 1661 | −4.07567 | 0 | 0.063907 | 0.256055 | 0 | −0.03007 | 0 | −0.21797 | 0.865937 |
| 1662 | −3.58253 | 0 | 0.054283 | 0.26327 | 0 | −0.07242 | 0 | −0.14884 | 0.712498 |
| 1663 | −3.54531 | 0 | 0.053803 | 0.254612 | 0 | −0.03643 | 0 | −0.18594 | 0.713422 |
| 1664 | −3.81334 | 0 | 0.058706 | 0.237478 | 0 | −0.07151 | 0 | −0.16789 | 0.756374 |
| 1665 | −3.7857 | 0 | 0.058549 | 0.230385 | 0 | −0.04159 | 0 | −0.20285 | 0.772659 |
| 1666 | −3.66263 | 0 | 0.056083 | 0.249917 | 0 | −0.0863 | 0 | 0 | 0.811302 |
| 1667 | −3.64627 | 0 | 0.055898 | 0.273963 | 0 | −0.12283 | 0 | 0 | 0.830371 |
| 1668 | −3.6115 | 0 | 0.055173 | 0.25626 | 0 | −0.09113 | 0 | 0 | 0.807489 |
| 1669 | −3.96116 | 0 | 0.061427 | 0.229275 | 0 | −0.11804 | 0 | 0 | 0.843134 |
| 1670 | −3.94067 | 0 | 0.061031 | 0.214899 | 0 | −0.09483 | 0 | 0 | 0.826742 |
| 1671 | −3.97971 | 0 | 0.06152 | 0.232609 | 0 | −0.11179 | 0 | 0 | 0.833523 |
| 1672 | −3.47853 | 0 | 0.052517 | 0.239684 | 0 | −0.11552 | 0 | 0 | 0.73897 |
| 1673 | −3.4577 | 0 | 0.052057 | 0.223915 | 0 | −0.09124 | 0 | 0 | 0.71732 |
| 1674 | −3.46136 | 0 | 0.052102 | 0.245015 | 0 | −0.11291 | 0 | 0 | 0.731513 |
| 1675 | −3.72851 | 0 | 0.056939 | 0.214464 | 0 | −0.114 | 0 | 0 | 0.759142 |
| 1676 | −3.88029 | 0 | 0.059483 | 0.267861 | 0 | 0 | −0.08568 | −0.19981 | 0.759561 |
| 1677 | −3.80095 | 0 | 0.05827 | 0.291206 | 0 | 0 | −0.06411 | −0.16247 | 0.793809 |
| 1678 | −3.65368 | 0 | 0.054877 | 0.25512 | 0 | 0 | −0.07672 | −0.14909 | 0.664468 |
| 1679 | −4.08726 | 0 | 0.063621 | 0.257026 | 0 | 0 | −0.07029 | −0.18164 | 0.83094 |
| 1680 | −4.12666 | 0 | 0.064151 | 0.27882 | 0 | 0 | −0.07244 | −0.18828 | 0.840583 |
| 1681 | −4.10358 | 0 | 0.064088 | 0.272688 | 0 | 0 | −0.07808 | −0.21916 | 0.860574 |
| 1682 | −3.6331 | 0 | 0.054694 | 0.280961 | 0 | 0 | −0.0787 | −0.15674 | 0.696421 |
| 1683 | −3.58019 | 0 | 0.054083 | 0.273303 | 0 | 0 | −0.08333 | −0.18909 | 0.708828 |
| 1684 | −3.84999 | 0 | 0.058899 | 0.255383 | 0 | 0 | −0.07838 | −0.17427 | 0.741204 |
| 1685 | −3.81075 | 0 | 0.05866 | 0.248587 | 0 | 0 | −0.08271 | −0.20575 | 0.767246 |
| 1686 | −3.685 | 0 | 0.056052 | 0.263985 | 0 | 0 | −0.0672 | 0 | 0.799388 |
| 1687 | −3.68404 | 0 | 0.055963 | 0.287287 | 0 | 0 | −0.06683 | 0 | 0.807665 |
| 1688 | −3.64392 | 0 | 0.055274 | 0.272634 | 0 | 0 | −0.07319 | 0 | 0.795404 |
| 1689 | −3.98592 | 0 | 0.061271 | 0.245814 | 0 | 0 | −0.07711 | 0 | 0.820242 |
| 1690 | −3.96248 | 0 | 0.06092 | 0.233391 | 0 | 0 | −0.08279 | 0 | 0.812799 |
| 1691 | −4.01401 | 0 | 0.061502 | 0.251265 | 0 | 0 | −0.08346 | 0 | 0.810813 |
| 1692 | −3.52584 | 0 | 0.052697 | 0.256591 | 0 | 0 | −0.08045 | 0 | 0.713538 |
| 1693 | −3.49264 | 0 | 0.052155 | 0.242953 | 0 | 0 | −0.08524 | 0 | 0.703088 |
| 1694 | −3.51566 | 0 | 0.052389 | 0.264012 | 0 | 0 | −0.08636 | 0 | 0.70733 |
| 1695 | −3.76783 | 0 | 0.056972 | 0.234186 | 0 | 0 | −0.08847 | 0 | 0.73446 |
| 1696 | −3.7537 | 0 | 0.058006 | 0.270052 | 0 | 0 | 0 | −0.17186 | 0.817433 |
| 1697 | −3.74343 | 0 | 0.057857 | 0.296161 | 0 | 0 | 0 | −0.17985 | 0.833113 |
| 1698 | −3.69853 | 0 | 0.057355 | 0.287037 | 0 | 0 | 0 | −0.21503 | 0.842307 |
| 1699 | −4.05163 | 0 | 0.063504 | 0.255845 | 0 | 0 | 0 | −0.19903 | 0.864829 |
| 1700 | −4.03126 | 0 | 0.063554 | 0.248337 | 0 | 0 | 0 | −0.23287 | 0.885348 |
| 1701 | −4.07149 | 0 | 0.063878 | 0.266566 | 0 | 0 | 0 | −0.22366 | 0.879849 |
| 1702 | −3.55569 | 0 | 0.054001 | 0.255812 | 0 | 0 | 0 | −0.1644 | 0.726427 |
| 1703 | −3.50127 | 0 | 0.053449 | 0.247039 | 0 | 0 | 0 | −0.19843 | 0.743319 |
| 1704 | −3.51593 | 0 | 0.053523 | 0.268441 | 0 | 0 | 0 | −0.19369 | 0.746727 |
| 1705 | −3.77985 | 0 | 0.058531 | 0.241251 | 0 | 0 | 0 | −0.21101 | 0.793108 |
| 1706 | −3.63772 | 0 | 0.055602 | 0.264483 | 0 | 0 | 0 | 0 | 0.822809 |
| 1707 | −3.59678 | 0 | 0.054955 | 0.248627 | 0 | 0 | 0 | 0 | 0.811885 |
| 1708 | −3.60775 | 0 | 0.054993 | 0.269582 | 0 | 0 | 0 | 0 | 0.816559 |
| 1709 | −3.93773 | 0 | 0.060793 | 0.224671 | 0 | 0 | 0 | 0 | 0.82915 |
| 1710 | −3.44285 | 0 | 0.051723 | 0.236135 | 0 | 0 | 0 | 0 | 0.730696 |
| 1711 | −3.51747 | 0 | 0.051983 | 0 | 0.138716 | −0.05833 | −0.06306 | −0.14172 | 0.583745 |
| 1712 | −3.88772 | 0 | 0.059865 | 0 | 0.14457 | −0.05624 | −0.05911 | −0.16711 | 0.779285 |
| 1713 | −3.67047 | 0 | 0.055459 | 0 | 0.124315 | −0.06145 | −0.06316 | −0.15537 | 0.667101 |
| 1714 | −3.68578 | 0 | 0.055778 | 0 | 0.136139 | −0.09261 | −0.0636 | −0.16097 | 0.686189 |
| 1715 | −3.66021 | 0 | 0.055574 | 0 | 0.127676 | −0.05901 | −0.06897 | −0.18977 | 0.700871 |
| 1716 | −3.62199 | 0 | 0.054415 | 0 | 0.082919 | −0.12249 | −0.0688 | 0 | 0.700975 |
| 1717 | −3.47441 | 0 | 0.052404 | 0 | 0.114227 | −0.09982 | −0.04984 | 0 | 0.757972 |
| 1718 | −3.38908 | 0 | 0.050156 | 0 | 0.09551 | −0.09349 | −0.06156 | 0 | 0.649915 |
| 1719 | −3.78842 | 0 | 0.058054 | 0 | 0.092503 | −0.09786 | −0.05942 | 0 | 0.790854 |
| 1720 | −3.80655 | 0 | 0.058272 | 0 | 0.099021 | −0.13041 | −0.05852 | 0 | 0.796238 |
| 1721 | −3.79695 | 0 | 0.058011 | 0 | 0.084721 | −0.09935 | −0.06476 | 0 | 0.783039 |
| 1722 | −3.35124 | 0 | 0.049654 | 0 | 0.100592 | −0.12831 | −0.05959 | 0 | 0.670609 |
| 1723 | −3.34677 | 0 | 0.049409 | 0 | 0.085622 | −0.09867 | −0.06501 | 0 | 0.649672 |
| 1724 | −3.58424 | 0 | 0.054057 | 0 | 0.085852 | −0.12743 | −0.06306 | 0 | 0.716926 |
| 1725 | −3.57518 | 0 | 0.053866 | 0 | 0.070007 | −0.10276 | −0.06817 | 0 | 0.707928 |
| 1726 | −3.61736 | 0 | 0.055399 | 0 | 0.118009 | −0.09015 | 0 | −0.19092 | 0.740722 |
| 1727 | −3.51544 | 0 | 0.05371 | 0 | 0.140436 | −0.06945 | 0 | −0.15546 | 0.765455 |
| 1728 | −3.42704 | 0 | 0.051322 | 0 | 0.116304 | −0.0652 | 0 | −0.14556 | 0.64517 |
| 1729 | −3.83171 | 0 | 0.059613 | 0 | 0.119249 | −0.06597 | 0 | −0.17474 | 0.813742 |
| 1730 | −3.83994 | 0 | 0.059696 | 0 | 0.133818 | −0.09965 | 0 | −0.17883 | 0.82325 |
| 1731 | −3.82287 | 0 | 0.059642 | 0 | 0.123474 | −0.06211 | 0 | −0.21007 | 0.836507 |
| 1732 | −3.37477 | 0 | 0.050632 | 0 | 0.126713 | −0.09953 | 0 | −0.1496 | 0.671669 |
| 1733 | −3.33905 | 0 | 0.050202 | 0 | 0.116097 | −0.06454 | 0 | −0.18169 | 0.677496 |
| 1734 | −3.60367 | 0 | 0.055148 | 0 | 0.113005 | −0.09787 | 0 | −0.16598 | 0.731872 |
| 1735 | −3.57748 | 0 | 0.055024 | 0 | 0.099855 | −0.06725 | 0 | −0.19704 | 0.750732 |
| 1736 | −3.44336 | 0 | 0.052367 | 0 | 0.09509 | −0.10864 | 0 | 0 | 0.783548 |
| 1737 | −3.41166 | 0 | 0.051869 | 0 | 0.100177 | −0.14593 | 0 | 0 | 0.79247 |
| 1738 | −3.3888 | 0 | 0.051373 | 0 | 0.082919 | −0.11329 | 0 | 0 | 0.776241 |
| 1739 | −3.75291 | 0 | 0.057925 | 0 | 0.078906 | −0.13704 | 0 | 0 | 0.822204 |
| 1740 | −3.741 | 0 | 0.057695 | 0 | 0.060507 | −0.11107 | 0 | 0 | 0.811145 |
| 1741 | −3.76778 | 0 | 0.057943 | 0 | 0.075138 | −0.13146 | 0 | 0 | 0.810251 |
| 1742 | −3.29249 | 0 | 0.049265 | 0 | 0.081611 | −0.13332 | 0 | 0 | 0.707824 |
| 1743 | −3.27812 | 0 | 0.048958 | 0 | 0.063381 | −0.10687 | 0 | 0 | 0.694084 |
| 1744 | −3.27529 | 0 | 0.048803 | 0 | 0.075232 | −0.12998 | 0 | 0 | 0.695017 |
| 1745 | −3.54375 | 0 | 0.053806 | 0 | 0.059601 | −0.12898 | 0 | 0 | 0.741141 |
| 1746 | −3.64315 | 0 | 0.055366 | 0 | 0.110243 | 0 | −0.06431 | −0.19868 | 0.723426 |
| 1747 | −3.53393 | 0 | 0.053721 | 0 | 0.134246 | 0 | −0.04715 | −0.16243 | 0.757432 |
| 1748 | −3.44195 | 0 | 0.051243 | 0 | 0.113496 | 0 | −0.05796 | −0.15168 | 0.635581 |
| 1749 | −3.84047 | 0 | 0.059464 | 0 | 0.115717 | 0 | −0.05322 | −0.1798 | 0.80606 |
| 1750 | −3.86184 | 0 | 0.059615 | 0 | 0.122346 | 0 | −0.05342 | −0.18705 | 0.805482 |
| 1751 | −3.83721 | 0 | 0.059572 | 0 | 0.120358 | 0 | −0.05952 | −0.21523 | 0.829358 |
| 1752 | −3.41301 | 0 | 0.050776 | 0 | 0.116292 | 0 | −0.0571 | −0.15974 | 0.649981 |
| 1753 | −3.35758 | 0 | 0.050181 | 0 | 0.113314 | 0 | −0.06194 | −0.1883 | 0.66948 |
| 1754 | −3.6263 | 0 | 0.055054 | 0 | 0.104055 | 0 | −0.05834 | −0.17443 | 0.712411 |
| 1755 | −3.58723 | 0 | 0.054856 | 0 | 0.097404 | 0 | −0.06274 | −0.20284 | 0.742752 |
| 1756 | −3.45307 | 0 | 0.052114 | 0 | 0.077906 | 0 | −0.0463 | 0 | 0.771026 |
| 1757 | −3.43843 | 0 | 0.051719 | 0 | 0.072552 | 0 | −0.04367 | 0 | 0.767941 |
| 1758 | −3.40453 | 0 | 0.051191 | 0 | 0.064402 | 0 | −0.04968 | 0 | 0.76293 |
| 1759 | −3.76394 | 0 | 0.057518 | 0 | 0.056623 | 0 | −0.05456 | 0 | 0.797973 |
| 1760 | −3.74619 | 0 | 0.05731 | 0 | 0.045186 | 0 | −0.05991 | 0 | 0.79657 |
| 1761 | −3.78388 | 0 | 0.057604 | 0 | 0.053657 | 0 | −0.06002 | 0 | 0.785582 |
| 1762 | −3.32298 | 0 | 0.049145 | 0 | 0.060501 | 0 | −0.05592 | 0 | 0.680103 |
| 1763 | −3.29237 | 0 | 0.048718 | 0 | 0.049243 | 0 | −0.06045 | 0 | 0.678699 |
| 1764 | −3.31014 | 0 | 0.048754 | 0 | 0.055051 | 0 | −0.06002 | 0 | 0.668022 |
| 1765 | −3.56224 | 0 | 0.053489 | 0 | 0.041091 | 0 | −0.06405 | 0 | 0.715223 |
| 1766 | −3.50759 | 0 | 0.053765 | 0 | 0.112387 | 0 | 0 | −0.16871 | 0.783884 |
| 1767 | −3.48239 | 0 | 0.053262 | 0 | 0.115217 | 0 | 0 | −0.17655 | 0.786788 |
| 1768 | −3.43472 | 0 | 0.052773 | 0 | 0.111359 | 0 | 0 | −0.20837 | 0.802335 |
| 1769 | −3.81172 | 0 | 0.059351 | 0 | 0.099545 | 0 | 0 | −0.19403 | 0.833455 |
| 1770 | −3.79054 | 0 | 0.05942 | 0 | 0.091001 | 0 | 0 | −0.22499 | 0.857927 |
| 1771 | −3.81796 | 0 | 0.059474 | 0 | 0.106025 | 0 | 0 | −0.2178 | 0.843714 |
| 1772 | −3.35396 | 0 | 0.050397 | 0 | 0.095738 | 0 | 0 | −0.16407 | 0.689153 |
| 1773 | −3.2972 | 0 | 0.049858 | 0 | 0.087955 | 0 | 0 | −0.19451 | 0.712892 |
| 1774 | −3.30713 | 0 | 0.049734 | 0 | 0.098044 | 0 | 0 | −0.19101 | 0.701538 |
| 1775 | −3.56908 | 0 | 0.054845 | 0 | 0.084201 | 0 | 0 | −0.20626 | 0.764589 |
| 1776 | −3.41366 | 0 | 0.051685 | 0 | 0.056384 | 0 | 0 | 0 | 0.787777 |
| 1777 | −3.37825 | 0 | 0.051183 | 0 | 0.043449 | 0 | 0 | 0 | 0.784339 |
| 1778 | −3.37973 | 0 | 0.050996 | 0 | 0.048586 | 0 | 0 | 0 | 0.778693 |
| 1779 | −3.73307 | 0 | 0.057298 | 0 | 0.0305 | 0 | 0 | 0 | 0.810476 |
| 1780 | −3.25558 | 0 | 0.048406 | 0 | 0.033948 | 0 | 0 | 0 | 0.70219 |
| 1781 | −3.6263 | 0 | 0.05525 | 0 | 0 | −0.06946 | −0.05365 | −0.17474 | 0.739542 |
| 1782 | −3.54296 | 0 | 0.053848 | 0 | 0 | −0.04497 | −0.03363 | −0.14077 | 0.759356 |
| 1783 | −3.43521 | 0 | 0.051197 | 0 | 0 | −0.0453 | −0.04602 | −0.13207 | 0.646153 |
| 1784 | −3.84305 | 0 | 0.059517 | 0 | 0 | −0.04528 | −0.04106 | −0.16124 | 0.812113 |
| 1785 | −3.85399 | 0 | 0.059602 | 0 | 0 | −0.07637 | −0.04187 | −0.16218 | 0.816896 |
| 1786 | −3.83817 | 0 | 0.059584 | 0 | 0 | −0.04055 | −0.04813 | −0.19429 | 0.833843 |
| 1787 | −3.38995 | 0 | 0.050582 | 0 | 0 | −0.07727 | −0.0453 | −0.13395 | 0.667869 |
| 1788 | −3.34921 | 0 | 0.050107 | 0 | 0 | −0.04454 | −0.05057 | −0.16716 | 0.678825 |
| 1789 | −3.60881 | 0 | 0.054969 | 0 | 0 | −0.07812 | −0.04723 | −0.15184 | 0.731752 |
| 1790 | −3.5783 | 0 | 0.054812 | 0 | 0 | −0.04969 | −0.05243 | −0.18443 | 0.75548 |
| 1791 | −3.46093 | 0 | 0.052444 | 0 | 0 | −0.08739 | −0.03557 | 0 | 0.779786 |
| 1792 | −3.42991 | 0 | 0.051962 | 0 | 0 | −0.12284 | −0.03332 | 0 | 0.787639 |
| 1793 | −3.40457 | 0 | 0.051443 | 0 | 0 | −0.09378 | −0.04059 | 0 | 0.774242 |
| 1794 | −3.75918 | 0 | 0.057821 | 0 | 0 | −0.11877 | −0.04599 | 0 | 0.819356 |
| 1795 | −3.74505 | 0 | 0.057573 | 0 | 0 | −0.09624 | −0.05276 | 0 | 0.81113 |
| 1796 | −3.7775 | 0 | 0.057889 | 0 | 0 | −0.11354 | −0.05258 | 0 | 0.807981 |
| 1797 | −3.30091 | 0 | 0.049201 | 0 | 0 | −0.11474 | −0.04714 | 0 | 0.705391 |
| 1798 | −3.28003 | 0 | 0.048818 | 0 | 0 | −0.09218 | −0.0534 | 0 | 0.695553 |
| 1799 | −3.28415 | 0 | 0.048754 | 0 | 0 | −0.11263 | −0.0524 | 0 | 0.694088 |
| 1800 | −3.54476 | 0 | 0.053638 | 0 | 0 | −0.11474 | −0.05751 | 0 | 0.741878 |
| 1801 | −3.51428 | 0 | 0.053869 | 0 | 0 | −0.0549 | 0 | −0.14837 | 0.788901 |
| 1802 | −3.47545 | 0 | 0.053268 | 0 | 0 | −0.08979 | 0 | −0.14987 | 0.79823 |
| 1803 | −3.44009 | 0 | 0.052864 | 0 | 0 | −0.05306 | 0 | −0.18661 | 0.806851 |
| 1804 | −3.80384 | 0 | 0.059346 | 0 | 0 | −0.08462 | 0 | −0.17153 | 0.846621 |
| 1805 | −3.78827 | 0 | 0.05942 | 0 | 0 | −0.05409 | 0 | −0.20668 | 0.866031 |
| 1806 | −3.81028 | 0 | 0.059441 | 0 | 0 | −0.0749 | 0 | −0.1938 | 0.854417 |
| 1807 | −3.33154 | 0 | 0.050222 | 0 | 0 | −0.08442 | 0 | −0.14038 | 0.708466 |
| 1808 | −3.28618 | 0 | 0.049769 | 0 | 0 | −0.05473 | 0 | −0.17574 | 0.724883 |
| 1809 | −3.28577 | 0 | 0.049548 | 0 | 0 | −0.0771 | 0 | −0.1668 | 0.717933 |
| 1810 | −3.55173 | 0 | 0.054741 | 0 | 0 | −0.07801 | 0 | −0.18537 | 0.782328 |
| 1811 | −3.40667 | 0 | 0.051944 | 0 | 0 | −0.13037 | 0 | 0 | 0.807952 |
| 1812 | −3.37651 | 0 | 0.051421 | 0 | 0 | −0.10617 | 0 | 0 | 0.797307 |
| 1813 | −3.36869 | 0 | 0.051197 | 0 | 0 | −0.12782 | 0 | 0 | 0.799008 |
| 1814 | −3.7261 | 0 | 0.057579 | 0 | 0 | −0.1247 | 0 | 0 | 0.832674 |
| 1815 | −3.23135 | 0 | 0.048436 | 0 | 0 | −0.12145 | 0 | 0 | 0.727677 |
| 1816 | −3.5238 | 0 | 0.053822 | 0 | 0 | 0 | −0.0303 | −0.15428 | 0.782631 |
| 1817 | −3.49759 | 0 | 0.053309 | 0 | 0 | 0 | −0.02999 | −0.16036 | 0.784641 |
| 1818 | −3.45345 | 0 | 0.052871 | 0 | 0 | 0 | −0.03524 | −0.19263 | 0.801349 |
| 1819 | −3.81625 | 0 | 0.059221 | 0 | 0 | 0 | −0.03845 | −0.17943 | 0.832875 |
| 1820 | −3.79513 | 0 | 0.059298 | 0 | 0 | 0 | −0.04332 | −0.21132 | 0.859676 |
| 1821 | −3.82636 | 0 | 0.05938 | 0 | 0 | 0 | −0.04498 | −0.20074 | 0.842227 |
| 1822 | −3.3566 | 0 | 0.050261 | 0 | 0 | 0 | −0.04306 | −0.14959 | 0.691368 |
| 1823 | −3.29699 | 0 | 0.04969 | 0 | 0 | 0 | −0.04727 | −0.18127 | 0.717777 |
| 1824 | −3.31209 | 0 | 0.049626 | 0 | 0 | 0 | −0.04785 | −0.17529 | 0.703793 |
| 1825 | −3.56726 | 0 | 0.054639 | 0 | 0 | 0 | −0.05024 | −0.1926 | 0.768525 |
| 1826 | −3.42347 | 0 | 0.051726 | 0 | 0 | 0 | −0.03382 | 0 | 0.786169 |
| 1827 | −3.3866 | 0 | 0.051194 | 0 | 0 | 0 | −0.04001 | 0 | 0.783506 |
| 1828 | −3.39099 | 0 | 0.05106 | 0 | 0 | 0 | −0.03891 | 0 | 0.777607 |
| 1829 | −3.73669 | 0 | 0.057212 | 0 | 0 | 0 | −0.05179 | 0 | 0.809086 |
| 1830 | −3.25981 | 0 | 0.048341 | 0 | 0 | 0 | −0.05198 | 0 | 0.702031 |
| 1831 | −3.47762 | 0 | 0.053356 | 0 | 0 | 0 | 0 | −0.16748 | 0.80591 |
| 1832 | −3.43332 | 0 | 0.052986 | 0 | 0 | 0 | 0 | −0.20233 | 0.825365 |
| 1833 | −3.42812 | 0 | 0.052641 | 0 | 0 | 0 | 0 | −0.19439 | 0.816058 |
| 1834 | −3.78129 | 0 | 0.05922 | 0 | 0 | 0 | 0 | −0.2139 | 0.870596 |
| 1835 | −3.2631 | 0 | 0.049387 | 0 | 0 | 0 | 0 | −0.18437 | 0.738794 |
| 1836 | −3.36813 | 0 | 0.051042 | 0 | 0 | 0 | 0 | 0 | 0.795368 |
| 1837 | −0.2123 | 0 | 0 | 0.157746 | 0.109461 | −0.03673 | −0.07604 | −0.11543 | 0.840193 |
| 1838 | −0.2201 | 0 | 0 | 0.123539 | 0.112868 | −0.02829 | −0.07637 | −0.13723 | 0.832769 |
| 1839 | −0.20079 | 0 | 0 | 0.125616 | 0.094239 | −0.03851 | −0.08229 | −0.13233 | 0.835879 |
| 1840 | −0.20578 | 0 | 0 | 0.138111 | 0.1014 | −0.07559 | −0.08022 | −0.13932 | 0.846985 |
| 1841 | −0.19385 | 0 | 0 | 0.131232 | 0.095799 | −0.03669 | −0.08811 | −0.17222 | 0.859458 |
| 1842 | −0.22893 | 0 | 0 | 0.126486 | 0.051945 | −0.09854 | −0.08572 | 0 | 0.851773 |
| 1843 | −0.20526 | 0 | 0 | 0.157003 | 0.090594 | −0.06875 | −0.06008 | 0 | 0.893979 |
| 1844 | −0.20545 | 0 | 0 | 0.146342 | 0.067841 | −0.06715 | −0.07554 | 0 | 0.870582 |
| 1845 | −0.21779 | 0 | 0 | 0.110875 | 0.064992 | −0.06387 | −0.0784 | 0 | 0.857903 |
| 1846 | −0.22764 | 0 | 0 | 0.123781 | 0.067563 | −0.09824 | −0.0761 | 0 | 0.86109 |
| 1847 | −0.2377 | 0 | 0 | 0.11145 | 0.057242 | −0.06471 | −0.08183 | 0 | 0.846387 |
| 1848 | −0.20193 | 0 | 0 | 0.167386 | 0.071169 | −0.11306 | −0.07111 | 0 | 0.892543 |
| 1849 | −0.21275 | 0 | 0 | 0.153212 | 0.055676 | −0.0752 | −0.07984 | 0 | 0.874246 |
| 1850 | −0.20645 | 0 | 0 | 0.127455 | 0.054168 | −0.1045 | −0.08178 | 0 | 0.869388 |
| 1851 | −0.21051 | 0 | 0 | 0.11527 | 0.039557 | −0.07611 | −0.0879 | 0 | 0.860754 |
| 1852 | −0.16524 | 0 | 0 | 0.125559 | 0.083525 | −0.0749 | 0 | −0.17311 | 0.887637 |
| 1853 | −0.16785 | 0 | 0 | 0.155456 | 0.112861 | −0.04763 | 0 | −0.12777 | 0.899743 |
| 1854 | −0.17174 | 0 | 0 | 0.140246 | 0.086233 | −0.04655 | 0 | −0.12125 | 0.867621 |
| 1855 | −0.17188 | 0 | 0 | 0.108807 | 0.089247 | −0.04276 | 0 | −0.1448 | 0.870803 |
| 1856 | −0.1786 | 0 | 0 | 0.122294 | 0.096981 | −0.07765 | 0 | −0.15015 | 0.880161 |
| 1857 | −0.16999 | 0 | 0 | 0.113471 | 0.091429 | −0.03923 | 0 | −0.17991 | 0.887379 |
| 1858 | −0.16475 | 0 | 0 | 0.161841 | 0.09325 | −0.09181 | 0 | −0.12858 | 0.896624 |
| 1859 | −0.15316 | 0 | 0 | 0.152372 | 0.084365 | −0.04739 | 0 | −0.16931 | 0.902814 |
| 1860 | −0.16076 | 0 | 0 | 0.12377 | 0.079744 | −0.08321 | 0 | −0.14539 | 0.882022 |
| 1861 | −0.14359 | 0 | 0 | 0.115174 | 0.069444 | −0.04948 | 0 | −0.17882 | 0.897297 |
| 1862 | −0.17541 | 0 | 0 | 0.139774 | 0.069589 | −0.08055 | 0 | 0 | 0.908687 |
| 1863 | −0.17342 | 0 | 0 | 0.161788 | 0.070703 | −0.12493 | 0 | 0 | 0.927188 |
| 1864 | −0.18415 | 0 | 0 | 0.145099 | 0.056382 | −0.08695 | 0 | 0 | 0.909717 |
| 1865 | −0.18932 | 0 | 0 | 0.10794 | 0.046999 | −0.10807 | 0 | 0 | 0.887272 |
| 1866 | −0.19341 | 0 | 0 | 0.094659 | 0.032709 | −0.08074 | 0 | 0 | 0.877871 |
| 1867 | −0.21023 | 0 | 0 | 0.106873 | 0.044426 | −0.10096 | 0 | 0 | 0.872412 |
| 1868 | −0.17148 | 0 | 0 | 0.148775 | 0.050261 | −0.11821 | 0 | 0 | 0.908214 |
| 1869 | −0.17681 | 0 | 0 | 0.133498 | 0.031699 | −0.08597 | 0 | 0 | 0.895318 |
| 1870 | −0.18232 | 0 | 0 | 0.152304 | 0.043478 | −0.11586 | 0 | 0 | 0.905036 |
| 1871 | −0.1861 | 0 | 0 | 0.109906 | 0.027646 | −0.1073 | 0 | 0 | 0.882944 |
| 1872 | −0.19141 | 0 | 0 | 0.144329 | 0.076772 | 0 | −0.08442 | −0.17826 | 0.873474 |
| 1873 | −0.18573 | 0 | 0 | 0.168287 | 0.110024 | 0 | −0.05926 | −0.13147 | 0.891219 |
| 1874 | −0.19096 | 0 | 0 | 0.154371 | 0.085378 | 0 | −0.07449 | −0.12485 | 0.860092 |
| 1875 | −0.18919 | 0 | 0 | 0.123963 | 0.089495 | 0 | −0.07622 | −0.14683 | 0.864411 |
| 1876 | −0.20354 | 0 | 0 | 0.139822 | 0.088497 | 0 | −0.07421 | −0.15488 | 0.864455 |
| 1877 | −0.18739 | 0 | 0 | 0.129908 | 0.091996 | 0 | −0.08019 | −0.1821 | 0.881845 |
| 1878 | −0.19428 | 0 | 0 | 0.177833 | 0.082272 | 0 | −0.07084 | −0.13663 | 0.876488 |
| 1879 | −0.1727 | 0 | 0 | 0.168491 | 0.083671 | 0 | −0.07913 | −0.17364 | 0.896875 |
| 1880 | −0.18803 | 0 | 0 | 0.141357 | 0.071372 | 0 | −0.08037 | −0.15121 | 0.865654 |
| 1881 | −0.1626 | 0 | 0 | 0.132291 | 0.068735 | 0 | −0.08663 | −0.18236 | 0.892258 |
| 1882 | −0.19968 | 0 | 0 | 0.15432 | 0.057765 | 0 | −0.06166 | 0 | 0.896776 |
| 1883 | −0.20833 | 0 | 0 | 0.176926 | 0.046492 | 0 | −0.05616 | 0 | 0.902827 |
| 1884 | −0.20972 | 0 | 0 | 0.160902 | 0.042822 | 0 | −0.06337 | 0 | 0.896941 |
| 1885 | −0.22186 | 0 | 0 | 0.126865 | 0.030582 | 0 | −0.07778 | 0 | 0.866691 |
| 1886 | −0.21898 | 0 | 0 | 0.113369 | 0.023211 | 0 | −0.08221 | 0 | 0.866365 |
| 1887 | −0.2422 | 0 | 0 | 0.126474 | 0.029463 | 0 | −0.07974 | 0 | 0.852323 |
| 1888 | −0.20817 | 0 | 0 | 0.164957 | 0.030789 | 0 | −0.07184 | 0 | 0.883794 |
| 1889 | −0.20431 | 0 | 0 | 0.150501 | 0.020659 | 0 | −0.07867 | 0 | 0.882951 |
| 1890 | −0.21892 | 0 | 0 | 0.169838 | 0.025456 | 0 | −0.07548 | 0 | 0.881128 |
| 1891 | −0.22081 | 0 | 0 | 0.129263 | 0.012492 | 0 | −0.08546 | 0 | 0.861632 |
| 1892 | −0.15578 | 0 | 0 | 0.1521 | 0.086282 | 0 | 0 | −0.13839 | 0.906367 |
| 1893 | −0.15926 | 0 | 0 | 0.176979 | 0.081788 | 0 | 0 | −0.14913 | 0.920768 |
| 1894 | −0.14379 | 0 | 0 | 0.164445 | 0.084708 | 0 | 0 | −0.18353 | 0.932858 |
| 1895 | −0.16462 | 0 | 0 | 0.125361 | 0.066041 | 0 | 0 | −0.16229 | 0.891966 |
| 1896 | −0.14338 | 0 | 0 | 0.114798 | 0.063056 | 0 | 0 | −0.1912 | 0.912556 |
| 1897 | −0.16942 | 0 | 0 | 0.127845 | 0.071625 | 0 | 0 | −0.18563 | 0.898359 |
| 1898 | −0.16253 | 0 | 0 | 0.160077 | 0.061135 | 0 | 0 | −0.14217 | 0.894239 |
| 1899 | −0.13757 | 0 | 0 | 0.149227 | 0.056983 | 0 | 0 | −0.17884 | 0.91556 |
| 1900 | −0.15391 | 0 | 0 | 0.168745 | 0.06294 | 0 | 0 | −0.17665 | 0.91565 |
| 1901 | −0.14775 | 0 | 0 | 0.128836 | 0.05122 | 0 | 0 | −0.18592 | 0.904905 |
| 1902 | −0.18461 | 0 | 0 | 0.16004 | 0.027956 | 0 | 0 | 0 | 0.913227 |
| 1903 | −0.18243 | 0 | 0 | 0.143105 | 0.018821 | 0 | 0 | 0 | 0.909641 |
| 1904 | −0.19424 | 0 | 0 | 0.163545 | 0.020769 | 0 | 0 | 0 | 0.911474 |
| 1905 | −0.20512 | 0 | 0 | 0.109648 | 0.004293 | 0 | 0 | 0 | 0.87686 |
| 1906 | −0.18904 | 0 | 0 | 0.149969 | 0.001503 | 0 | 0 | 0 | 0.896885 |
| 1907 | −0.18126 | 0 | 0 | 0.149259 | 0 | −0.05693 | −0.07754 | −0.16063 | 0.889978 |
| 1908 | −0.18739 | 0 | 0 | 0.176712 | 0 | −0.02309 | −0.04891 | −0.11471 | 0.892581 |
| 1909 | −0.18815 | 0 | 0 | 0.130814 | 0 | −0.02337 | −0.06775 | −0.13319 | 0.870804 |
| 1910 | −0.18815 | 0 | 0 | 0.160586 | 0 | −0.02817 | −0.06669 | −0.11017 | 0.868123 |
| 1911 | −0.19564 | 0 | 0 | 0.145919 | 0 | −0.05674 | −0.06596 | −0.1364 | 0.878055 |
| 1912 | −0.18735 | 0 | 0 | 0.137596 | 0 | −0.01891 | −0.07227 | −0.16709 | 0.887266 |
| 1913 | −0.18189 | 0 | 0 | 0.182973 | 0 | −0.07171 | −0.06243 | −0.11585 | 0.894227 |
| 1914 | −0.16948 | 0 | 0 | 0.174626 | 0 | −0.02919 | −0.07168 | −0.15789 | 0.904619 |
| 1915 | −0.17551 | 0 | 0 | 0.14504 | 0 | −0.066 | −0.07331 | −0.13384 | 0.884001 |
| 1916 | −0.15723 | 0 | 0 | 0.136597 | 0 | −0.03401 | −0.08034 | −0.16895 | 0.90288 |
| 1917 | −0.18959 | 0 | 0 | 0.157734 | 0 | −0.0614 | −0.05374 | 0 | 0.904266 |
| 1918 | −0.18643 | 0 | 0 | 0.178815 | 0 | −0.10539 | −0.04781 | 0 | 0.922236 |
| 1919 | −0.19738 | 0 | 0 | 0.162501 | 0 | −0.0704 | −0.05672 | 0 | 0.906455 |
| 1920 | −0.20133 | 0 | 0 | 0.126756 | 0 | −0.09471 | −0.07258 | 0 | 0.886916 |
| 1921 | −0.20538 | 0 | 0 | 0.112887 | 0 | −0.07036 | −0.07828 | 0 | 0.878642 |
| 1922 | −0.22323 | 0 | 0 | 0.12661 | 0 | −0.08812 | −0.075 | 0 | 0.871669 |
| 1923 | −0.18438 | 0 | 0 | 0.165841 | 0 | −0.10423 | −0.0661 | 0 | 0.906225 |
| 1924 | −0.18903 | 0 | 0 | 0.150402 | 0 | −0.0764 | −0.07494 | 0 | 0.895786 |
| 1925 | −0.19549 | 0 | 0 | 0.170375 | 0 | −0.10369 | −0.07041 | 0 | 0.903777 |
| 1926 | −0.19798 | 0 | 0 | 0.127556 | 0 | −0.09881 | −0.08231 | 0 | 0.884065 |
| 1927 | −0.15515 | 0 | 0 | 0.159292 | 0 | −0.03734 | 0 | −0.12312 | 0.911269 |
| 1928 | −0.14978 | 0 | 0 | 0.182955 | 0 | −0.0796 | 0 | −0.12796 | 0.933983 |
| 1929 | −0.14301 | 0 | 0 | 0.171363 | 0 | −0.03673 | 0 | −0.16701 | 0.937168 |
| 1930 | −0.15442 | 0 | 0 | 0.129163 | 0 | −0.06918 | 0 | −0.14598 | 0.907512 |
| 1931 | −0.13948 | 0 | 0 | 0.119069 | 0 | −0.03843 | 0 | −0.17853 | 0.921378 |
| 1932 | −0.1618 | 0 | 0 | 0.132955 | 0 | −0.0587 | 0 | −0.16883 | 0.911629 |
| 1933 | −0.14907 | 0 | 0 | 0.163786 | 0 | −0.07947 | 0 | −0.1235 | 0.912382 |
| 1934 | −0.13193 | 0 | 0 | 0.153004 | 0 | −0.04315 | 0 | −0.1653 | 0.925063 |
| 1935 | −0.14222 | 0 | 0 | 0.172978 | 0 | −0.07263 | 0 | −0.1581 | 0.931737 |
| 1936 | −0.13545 | 0 | 0 | 0.130944 | 0 | −0.06851 | 0 | −0.17085 | 0.921994 |
| 1937 | −0.16199 | 0 | 0 | 0.161223 | 0 | −0.115 | 0 | 0 | 0.933484 |
| 1938 | −0.16766 | 0 | 0 | 0.14298 | 0 | −0.08708 | 0 | 0 | 0.921407 |
| 1939 | −0.1725 | 0 | 0 | 0.163765 | 0 | −0.11306 | 0 | 0 | 0.930913 |
| 1940 | −0.18393 | 0 | 0 | 0.106911 | 0 | −0.1031 | 0 | 0 | 0.896815 |
| 1941 | −0.16453 | 0 | 0 | 0.148476 | 0 | −0.11374 | 0 | 0 | 0.918668 |
| 1942 | −0.16967 | 0 | 0 | 0.169975 | 0 | 0 | −0.05072 | −0.12578 | 0.90415 |
| 1943 | −0.17164 | 0 | 0 | 0.193755 | 0 | 0 | −0.04627 | −0.13597 | 0.91829 |
| 1944 | −0.15782 | 0 | 0 | 0.182597 | 0 | 0 | −0.05274 | −0.16995 | 0.93044 |
| 1945 | −0.176 | 0 | 0 | 0.144431 | 0 | 0 | −0.06892 | −0.15077 | 0.894391 |
| 1946 | −0.15526 | 0 | 0 | 0.134226 | 0 | 0 | −0.07359 | −0.18036 | 0.916166 |
| 1947 | −0.18219 | 0 | 0 | 0.148608 | 0 | 0 | −0.07098 | −0.17277 | 0.900204 |
| 1948 | −0.17417 | 0 | 0 | 0.177373 | 0 | 0 | −0.06486 | −0.13145 | 0.895862 |
| 1949 | −0.14929 | 0 | 0 | 0.167451 | 0 | 0 | −0.07193 | −0.16907 | 0.919466 |
| 1950 | −0.16619 | 0 | 0 | 0.187778 | 0 | 0 | −0.06849 | −0.16546 | 0.917774 |
| 1951 | −0.15845 | 0 | 0 | 0.147728 | 0 | 0 | −0.0793 | −0.17632 | 0.910149 |
| 1952 | −0.19424 | 0 | 0 | 0.173068 | 0 | 0 | −0.054 | 0 | 0.910488 |
| 1953 | −0.19304 | 0 | 0 | 0.156709 | 0 | 0 | −0.061 | 0 | 0.907246 |
| 1954 | −0.20448 | 0 | 0 | 0.176278 | 0 | 0 | −0.05602 | 0 | 0.908266 |
| 1955 | −0.21598 | 0 | 0 | 0.124386 | 0 | 0 | −0.07923 | 0 | 0.87565 |
| 1956 | −0.20049 | 0 | 0 | 0.163398 | 0 | 0 | −0.07383 | 0 | 0.894251 |
| 1957 | −0.14682 | 0 | 0 | 0.176984 | 0 | 0 | 0 | −0.14382 | 0.929788 |
| 1958 | −0.12819 | 0 | 0 | 0.164268 | 0 | 0 | 0 | −0.17962 | 0.945155 |
| 1959 | −0.14137 | 0 | 0 | 0.185455 | 0 | 0 | 0 | −0.17469 | 0.946993 |
| 1960 | −0.14177 | 0 | 0 | 0.130707 | 0 | 0 | 0 | −0.18538 | 0.92652 |
| 1961 | −0.1345 | 0 | 0 | 0.16672 | 0 | 0 | 0 | −0.17413 | 0.932774 |
| 1962 | −0.18065 | 0 | 0 | 0.156574 | 0 | 0 | 0 | 0 | 0.917644 |
| 1963 | −0.18711 | 0 | 0 | 0 | 0.108401 | −0.08349 | −0.06842 | −0.16657 | 0.866319 |
| 1964 | −0.19054 | 0 | 0 | 0 | 0.137762 | −0.05788 | −0.04128 | −0.1216 | 0.870545 |
| 1965 | −0.18832 | 0 | 0 | 0 | 0.112184 | −0.051 | −0.06223 | −0.13887 | 0.857127 |
| 1966 | −0.19616 | 0 | 0 | 0 | 0.110812 | −0.05503 | −0.05835 | −0.11673 | 0.842144 |
| 1967 | −0.19744 | 0 | 0 | 0 | 0.12111 | −0.08628 | −0.05844 | −0.14394 | 0.859747 |
| 1968 | −0.18768 | 0 | 0 | 0 | 0.115149 | −0.04835 | −0.06533 | −0.17285 | 0.871772 |
| 1969 | −0.19387 | 0 | 0 | 0 | 0.119192 | −0.09961 | −0.05166 | −0.12393 | 0.8594 |
| 1970 | −0.18059 | 0 | 0 | 0 | 0.110954 | −0.05719 | −0.06095 | −0.1624 | 0.872723 |
| 1971 | −0.18147 | 0 | 0 | 0 | 0.103494 | −0.09124 | −0.065 | −0.1399 | 0.861904 |
| 1972 | −0.16236 | 0 | 0 | 0 | 0.093166 | −0.05829 | −0.07225 | −0.17241 | 0.883171 |
| 1973 | −0.19436 | 0 | 0 | 0 | 0.094466 | −0.08796 | −0.04427 | 0 | 0.885467 |
| 1974 | −0.19439 | 0 | 0 | 0 | 0.097004 | −0.13238 | −0.03691 | 0 | 0.895357 |
| 1975 | −0.20327 | 0 | 0 | 0 | 0.082091 | −0.095 | −0.04545 | 0 | 0.884704 |
| 1976 | −0.20466 | 0 | 0 | 0 | 0.071051 | −0.11462 | −0.06347 | 0 | 0.87255 |
| 1977 | −0.20738 | 0 | 0 | 0 | 0.055802 | −0.08712 | −0.06937 | 0 | 0.868017 |
| 1978 | −0.22614 | 0 | 0 | 0 | 0.069134 | −0.10759 | −0.06545 | 0 | 0.856791 |
| 1979 | −0.1959 | 0 | 0 | 0 | 0.076036 | −0.12415 | −0.05401 | 0 | 0.878202 |
| 1980 | −0.19849 | 0 | 0 | 0 | 0.057506 | −0.09277 | −0.06255 | 0 | 0.873293 |
| 1981 | −0.20808 | 0 | 0 | 0 | 0.071175 | −0.12204 | −0.05692 | 0 | 0.872543 |
| 1982 | −0.20381 | 0 | 0 | 0 | 0.052169 | −0.1136 | −0.07159 | 0 | 0.867575 |
| 1983 | −0.15981 | 0 | 0 | 0 | 0.117265 | −0.06665 | 0 | −0.12742 | 0.890866 |
| 1984 | −0.15842 | 0 | 0 | 0 | 0.125246 | −0.11061 | 0 | −0.13358 | 0.90492 |
| 1985 | −0.1496 | 0 | 0 | 0 | 0.116915 | −0.06763 | 0 | −0.17 | 0.912745 |
| 1986 | −0.15755 | 0 | 0 | 0 | 0.099569 | −0.09363 | 0 | −0.15053 | 0.890923 |
| 1987 | −0.14164 | 0 | 0 | 0 | 0.088519 | −0.06156 | 0 | −0.18107 | 0.907219 |
| 1988 | −0.1649 | 0 | 0 | 0 | 0.104368 | −0.08481 | 0 | −0.17378 | 0.893689 |
| 1989 | −0.16108 | 0 | 0 | 0 | 0.09881 | −0.10283 | 0 | −0.12871 | 0.882752 |
| 1990 | −0.14269 | 0 | 0 | 0 | 0.085932 | −0.06533 | 0 | −0.16712 | 0.899428 |
| 1991 | −0.15663 | 0 | 0 | 0 | 0.100849 | −0.09682 | 0 | −0.16194 | 0.897471 |
| 1992 | −0.14247 | 0 | 0 | 0 | 0.083058 | −0.08957 | 0 | −0.17368 | 0.901516 |
| 1993 | −0.171 | 0 | 0 | 0 | 0.080566 | −0.1369 | 0 | 0 | 0.909787 |
| 1994 | −0.17473 | 0 | 0 | 0 | 0.061431 | −0.10521 | 0 | 0 | 0.903075 |
| 1995 | −0.18203 | 0 | 0 | 0 | 0.073254 | −0.1336 | 0 | 0 | 0.905284 |
| 1996 | −0.18761 | 0 | 0 | 0 | 0.044852 | −0.11635 | 0 | 0 | 0.885571 |
| 1997 | −0.17669 | 0 | 0 | 0 | 0.048193 | −0.12642 | 0 | 0 | 0.894336 |
| 1998 | −0.17791 | 0 | 0 | 0 | 0.109906 | 0 | −0.04179 | −0.13278 | 0.880695 |
| 1999 | −0.18524 | 0 | 0 | 0 | 0.106908 | 0 | −0.03488 | −0.14293 | 0.884281 |
| 2000 | −0.16765 | 0 | 0 | 0 | 0.108889 | 0 | −0.04256 | −0.17572 | 0.903113 |
| 2001 | −0.18382 | 0 | 0 | 0 | 0.089138 | 0 | −0.05928 | −0.15624 | 0.872785 |
| 2002 | −0.16082 | 0 | 0 | 0 | 0.085397 | 0 | −0.06512 | −0.1846 | 0.898518 |
| 2003 | −0.18991 | 0 | 0 | 0 | 0.095224 | 0 | −0.06111 | −0.17868 | 0.876878 |
| 2004 | −0.19063 | 0 | 0 | 0 | 0.085752 | 0 | −0.05203 | −0.13748 | 0.861319 |
| 2005 | −0.16326 | 0 | 0 | 0 | 0.081333 | 0 | −0.05975 | −0.17268 | 0.890147 |
| 2006 | −0.1849 | 0 | 0 | 0 | 0.089076 | 0 | −0.05441 | −0.17004 | 0.87819 |
| 2007 | −0.16991 | 0 | 0 | 0 | 0.074514 | 0 | −0.06792 | −0.17957 | 0.884548 |
| 2008 | −0.20603 | 0 | 0 | 0 | 0.053677 | 0 | −0.03885 | 0 | 0.883872 |
| 2009 | −0.20153 | 0 | 0 | 0 | 0.043369 | 0 | −0.04615 | 0 | 0.887108 |
| 2010 | −0.2159 | 0 | 0 | 0 | 0.047208 | 0 | −0.04024 | 0 | 0.880355 |
| 2011 | −0.2213 | 0 | 0 | 0 | 0.028054 | 0 | −0.06599 | 0 | 0.862407 |
| 2012 | −0.21373 | 0 | 0 | 0 | 0.02787 | 0 | −0.05678 | 0 | 0.868368 |
| 2013 | −0.16117 | 0 | 0 | 0 | 0.089925 | 0 | 0 | −0.14781 | 0.899482 |
| 2014 | −0.13923 | 0 | 0 | 0 | 0.085618 | 0 | 0 | −0.1819 | 0.920733 |
| 2015 | −0.15666 | 0 | 0 | 0 | 0.092061 | 0 | 0 | −0.17786 | 0.913682 |
| 2016 | −0.15012 | 0 | 0 | 0 | 0.069702 | 0 | 0 | −0.18752 | 0.906986 |
| 2017 | −0.15224 | 0 | 0 | 0 | 0.066066 | 0 | 0 | −0.17547 | 0.900866 |
| 2018 | −0.19271 | 0 | 0 | 0 | 0.027816 | 0 | 0 | 0 | 0.894888 |
| 2019 | −5.27999 | 1.498105 | 0.066079 | 0.195356 | 0.004613 | −0.1273 | 0 | 0 | 0.553525 |
| 2020 | −5.58925 | 1.389893 | 0.072768 | 0.190717 | 0.008007 | −0.12041 | 0 | 0 | 0.631842 |
| 2021 | −5.46237 | 1.458937 | 0.069534 | 0.175223 | −0.00958 | −0.12644 | 0 | 0 | 0.552574 |
| 2022 | −5.44309 | 1.435857 | 0.069439 | 0.187406 | −0.00361 | −0.14882 | 0 | 0 | 0.559825 |
| 2023 | −5.45487 | 1.459584 | 0.069374 | 0.180652 | −0.01679 | −0.12709 | 0 | 0 | 0.555616 |
| 2024 | −5.28423 | 1.474912 | 0.065859 | 0.212838 | −0.01023 | 0 | −0.07981 | 0 | 0.542789 |
| 2025 | −5.58438 | 1.368455 | 0.072433 | 0.20759 | −0.00544 | 0 | −0.07936 | 0 | 0.62489 |
| 2026 | −5.45683 | 1.437263 | 0.069151 | 0.193921 | −0.02292 | 0 | −0.08177 | 0 | 0.543254 |
| 2027 | −5.46017 | 1.414241 | 0.06924 | 0.206037 | −0.02311 | 0 | −0.08293 | 0 | 0.534151 |
| 2028 | −5.45706 | 1.437003 | 0.069109 | 0.199554 | −0.03058 | 0 | −0.0858 | 0 | 0.545913 |
| 2029 | −5.36381 | 1.485589 | 0.067778 | 0.215019 | 0.019673 | 0 | 0 | −0.16107 | 0.543317 |
| 2030 | −5.67893 | 1.377065 | 0.074966 | 0.216091 | 0.033409 | 0 | 0 | −0.18945 | 0.661399 |
| 2031 | −5.52815 | 1.44733 | 0.071145 | 0.197493 | 0.008501 | 0 | 0 | −0.17584 | 0.562816 |
| 2032 | −5.51625 | 1.420399 | 0.071134 | 0.211694 | 0.016231 | 0 | 0 | −0.18195 | 0.564447 |
| 2033 | −5.49152 | 1.437245 | 0.070989 | 0.208764 | 0.009222 | 0 | 0 | −0.20068 | 0.601533 |
| 2034 | −5.20389 | 1.463889 | 0.065232 | 0.196574 | −0.03362 | 0 | 0 | 0 | 0.583409 |
| 2035 | −5.26612 | 1.381882 | 0.067675 | 0.228164 | −0.01249 | 0 | 0 | 0 | 0.686405 |
| 2036 | −5.51409 | 1.355821 | 0.071924 | 0.191884 | −0.03078 | 0 | 0 | 0 | 0.654353 |
| 2037 | −5.49546 | 1.333987 | 0.071649 | 0.204873 | −0.02967 | 0 | 0 | 0 | 0.649669 |
| 2038 | −5.50336 | 1.360854 | 0.071623 | 0.197404 | −0.03865 | 0 | 0 | 0 | 0.654032 |
| 2039 | −5.15356 | 1.434839 | 0.064582 | 0.213493 | −0.03502 | 0 | 0 | 0 | 0.582142 |
| 2040 | −5.14472 | 1.464367 | 0.064283 | 0.205837 | −0.04482 | 0 | 0 | 0 | 0.591241 |
| 2041 | −5.36708 | 1.405058 | 0.068441 | 0.189509 | −0.04503 | 0 | 0 | 0 | 0.57877 |
| 2042 | −5.36288 | 1.425056 | 0.06835 | 0.183062 | −0.05631 | 0 | 0 | 0 | 0.590865 |
| 2043 | −5.38509 | 1.413277 | 0.068521 | 0.193572 | −0.04833 | 0 | 0 | 0 | 0.570958 |
| 2044 | −5.31692 | 1.496805 | 0.06645 | 0.207388 | 0 | −0.12221 | −0.07569 | 0 | 0.545387 |
| 2045 | −5.61974 | 1.389849 | 0.073031 | 0.202385 | 0 | −0.11442 | −0.07498 | 0 | 0.624067 |
| 2046 | −5.48637 | 1.45516 | 0.069728 | 0.18731 | 0 | −0.12346 | −0.0786 | 0 | 0.545628 |
| 2047 | −5.47686 | 1.433296 | 0.069776 | 0.199425 | 0 | −0.14528 | −0.07931 | 0 | 0.551737 |
| 2048 | −5.48058 | 1.453565 | 0.069618 | 0.19218 | 0 | −0.12553 | −0.08336 | 0 | 0.54922 |
| 2049 | −5.39106 | 1.506711 | 0.068156 | 0.212877 | 0 | −0.08998 | 0 | −0.1466 | 0.543871 |
| 2050 | −5.70928 | 1.398392 | 0.075316 | 0.215333 | 0 | −0.07667 | 0 | −0.17544 | 0.656503 |
| 2051 | −5.55162 | 1.464675 | 0.071493 | 0.194509 | 0 | −0.08814 | 0 | −0.16294 | 0.562921 |
| 2052 | −5.53296 | 1.441008 | 0.071453 | 0.208878 | 0 | −0.10896 | 0 | −0.16536 | 0.573909 |
| 2053 | −5.51362 | 1.45484 | 0.071303 | 0.205809 | 0 | −0.08428 | 0 | −0.18772 | 0.600719 |
| 2054 | −5.23229 | 1.482076 | 0.065828 | 0.19032 | 0 | −0.13441 | 0 | 0 | 0.588533 |
| 2055 | −5.30586 | 1.405269 | 0.06839 | 0.224011 | 0 | −0.13333 | 0 | 0 | 0.690474 |
| 2056 | −5.54534 | 1.373143 | 0.072547 | 0.185804 | 0 | −0.13016 | 0 | 0 | 0.657487 |
| 2057 | −5.52269 | 1.352448 | 0.072345 | 0.198868 | 0 | −0.15284 | 0 | 0 | 0.66457 |
| 2058 | −5.52931 | 1.376754 | 0.072187 | 0.190155 | 0 | −0.13136 | 0 | 0 | 0.657362 |
| 2059 | −5.16333 | 1.455052 | 0.065055 | 0.208092 | 0 | −0.16222 | 0 | 0 | 0.603589 |
| 2060 | −5.165 | 1.480976 | 0.064789 | 0.198469 | 0 | −0.14024 | 0 | 0 | 0.597325 |
| 2061 | −5.38312 | 1.420424 | 0.069015 | 0.181883 | 0 | −0.15649 | 0 | 0 | 0.595848 |
| 2062 | −5.38217 | 1.43766 | 0.068864 | 0.174018 | 0 | −0.14016 | 0 | 0 | 0.595012 |
| 2063 | −5.39785 | 1.428133 | 0.069035 | 0.185616 | 0 | −0.15312 | 0 | 0 | 0.587037 |
| 2064 | −5.40445 | 1.488673 | 0.068166 | 0.228605 | 0 | 0 | −0.07581 | −0.15661 | 0.535986 |
| 2065 | −5.71482 | 1.38321 | 0.075237 | 0.229253 | 0 | 0 | −0.07138 | −0.18249 | 0.651998 |
| 2066 | −5.55539 | 1.448461 | 0.07135 | 0.211239 | 0 | 0 | −0.07697 | −0.17202 | 0.557039 |
| 2067 | −5.55354 | 1.422955 | 0.071477 | 0.225607 | 0 | 0 | −0.07838 | −0.17713 | 0.557351 |
| 2068 | −5.52135 | 1.437484 | 0.071246 | 0.222679 | 0 | 0 | −0.08148 | −0.19666 | 0.596926 |
| 2069 | −5.22956 | 1.455856 | 0.0655 | 0.20713 | 0 | 0 | −0.07876 | 0 | 0.575965 |
| 2070 | −5.2981 | 1.379218 | 0.068024 | 0.237308 | 0 | 0 | −0.06878 | 0 | 0.680111 |
| 2071 | −5.53186 | 1.348996 | 0.072073 | 0.202072 | 0 | 0 | −0.07794 | 0 | 0.648043 |
| 2072 | −5.5224 | 1.327174 | 0.071938 | 0.214502 | 0 | 0 | −0.07847 | 0 | 0.641734 |
| 2073 | −5.52468 | 1.351372 | 0.071839 | 0.206849 | 0 | 0 | −0.08277 | 0 | 0.646952 |
| 2074 | −5.18932 | 1.426092 | 0.065021 | 0.223752 | 0 | 0 | −0.0792 | 0 | 0.573906 |
| 2075 | −5.17307 | 1.452944 | 0.064618 | 0.21571 | 0 | 0 | −0.08302 | 0 | 0.583395 |
| 2076 | −5.39062 | 1.394657 | 0.068685 | 0.199809 | 0 | 0 | −0.08283 | 0 | 0.569604 |
| 2077 | −5.38003 | 1.411848 | 0.068505 | 0.192818 | 0 | 0 | −0.08656 | 0 | 0.581261 |
| 2078 | −5.41276 | 1.40117 | 0.068836 | 0.203618 | 0 | 0 | −0.08699 | 0 | 0.561176 |
| 2079 | −5.31561 | 1.473408 | 0.067507 | 0.211969 | 0 | 0 | 0 | −0.16471 | 0.582395 |
| 2080 | −5.41506 | 1.398402 | 0.070611 | 0.249609 | 0 | 0 | 0 | −0.17766 | 0.699539 |
| 2081 | −5.64326 | 1.365185 | 0.074833 | 0.213965 | 0 | 0 | 0 | −0.19367 | 0.688096 |
| 2082 | −5.62358 | 1.341149 | 0.074636 | 0.229634 | 0 | 0 | 0 | −0.19834 | 0.690363 |
| 2083 | −5.6165 | 1.358464 | 0.074735 | 0.226212 | 0 | 0 | 0 | −0.22045 | 0.720897 |
| 2084 | −5.25754 | 1.442916 | 0.066852 | 0.23119 | 0 | 0 | 0 | −0.1706 | 0.590038 |
| 2085 | −5.2149 | 1.463598 | 0.066331 | 0.227469 | 0 | 0 | 0 | −0.1925 | 0.621523 |
| 2086 | −5.45802 | 1.409797 | 0.070717 | 0.208569 | 0 | 0 | 0 | −0.18583 | 0.604159 |
| 2087 | −5.4261 | 1.42025 | 0.070547 | 0.204757 | 0 | 0 | 0 | −0.20757 | 0.640892 |
| 2088 | −5.45386 | 1.409818 | 0.070774 | 0.217163 | 0 | 0 | 0 | −0.20256 | 0.621591 |
| 2089 | −5.24647 | 1.364337 | 0.067693 | 0.219788 | 0 | 0 | 0 | 0 | 0.696982 |
| 2090 | −5.18609 | 1.330653 | 0.066903 | 0.23672 | 0 | 0 | 0 | 0 | 0.693976 |
| 2091 | −5.18158 | 1.36226 | 0.066623 | 0.227573 | 0 | 0 | 0 | 0 | 0.696692 |
| 2092 | −5.45098 | 1.314298 | 0.071376 | 0.195754 | 0 | 0 | 0 | 0 | 0.666792 |
| 2093 | −5.44987 | 1.334097 | 0.071284 | 0.187648 | 0 | 0 | 0 | 0 | 0.669919 |
| 2094 | −5.45778 | 1.324235 | 0.071267 | 0.199729 | 0 | 0 | 0 | 0 | 0.661429 |
| 2095 | −5.11585 | 1.41525 | 0.06441 | 0.203898 | 0 | 0 | 0 | 0 | 0.603483 |
| 2096 | −5.1 | 1.437942 | 0.064075 | 0.195443 | 0 | 0 | 0 | 0 | 0.61161 |
| 2097 | −5.08828 | 1.421482 | 0.063839 | 0.209969 | 0 | 0 | 0 | 0 | 0.601561 |
| 2098 | −5.32009 | 1.388338 | 0.068045 | 0.183292 | 0 | 0 | 0 | 0 | 0.597583 |
| 2099 | −5.18419 | 1.513848 | 0.063929 | 0 | 0.03121 | −0.13447 | −0.0604 | 0 | 0.516078 |
| 2100 | −5.46486 | 1.408253 | 0.070197 | 0 | 0.035146 | −0.12782 | −0.06112 | 0 | 0.60227 |
| 2101 | −5.34704 | 1.472128 | 0.067212 | 0 | 0.018663 | −0.1331 | −0.06257 | 0 | 0.531926 |
| 2102 | −5.33688 | 1.454275 | 0.067149 | 0 | 0.023292 | −0.1552 | −0.06359 | 0 | 0.529122 |
| 2103 | −5.33723 | 1.472823 | 0.066987 | 0 | 0.011621 | −0.13407 | −0.06724 | 0 | 0.532041 |
| 2104 | −5.23853 | 1.515553 | 0.065331 | 0 | 0.05586 | −0.10632 | 0 | −0.1394 | 0.512891 |
| 2105 | −5.524 | 1.408993 | 0.071999 | 0 | 0.068172 | −0.0954 | 0 | −0.16705 | 0.630934 |
| 2106 | −5.39018 | 1.473674 | 0.06862 | 0 | 0.044902 | −0.10259 | 0 | −0.15452 | 0.54609 |
| 2107 | −5.36981 | 1.452762 | 0.068462 | 0 | 0.055422 | −0.12462 | 0 | −0.15737 | 0.547359 |
| 2108 | −5.34371 | 1.465451 | 0.068214 | 0 | 0.045201 | −0.09954 | 0 | −0.17679 | 0.575984 |
| 2109 | −5.10273 | 1.498135 | 0.063373 | 0 | 0.013319 | −0.14096 | 0 | 0 | 0.566179 |
| 2110 | −5.14417 | 1.422288 | 0.065367 | 0 | 0.035312 | −0.14472 | 0 | 0 | 0.657208 |
| 2111 | −5.39926 | 1.39035 | 0.069854 | 0 | 0.015388 | −0.13725 | 0 | 0 | 0.640282 |
| 2112 | −5.37426 | 1.373264 | 0.069546 | 0 | 0.021759 | −0.16091 | 0 | 0 | 0.640453 |
| 2113 | −5.37866 | 1.395384 | 0.069394 | 0 | 0.00795 | −0.13762 | 0 | 0 | 0.637859 |
| 2114 | −5.03668 | 1.475988 | 0.062525 | 0 | 0.017656 | −0.1684 | 0 | 0 | 0.569856 |
| 2115 | −5.03075 | 1.499228 | 0.062206 | 0 | 0.003078 | −0.14488 | 0 | 0 | 0.570947 |
| 2116 | −5.25124 | 1.439635 | 0.066538 | 0 | 0.005671 | −0.1611 | 0 | 0 | 0.579621 |
| 2117 | −5.24533 | 1.455629 | 0.066355 | 0 | −0.0092 | −0.1423 | 0 | 0 | 0.585195 |
| 2118 | −5.26314 | 1.448449 | 0.066476 | 0 | 0.001791 | −0.15702 | 0 | 0 | 0.567565 |
| 2119 | −5.24037 | 1.499056 | 0.065125 | 0 | 0.046959 | 0 | −0.05933 | −0.15035 | 0.503628 |
| 2120 | −5.51963 | 1.39512 | 0.071737 | 0 | 0.060846 | 0 | −0.05724 | −0.1757 | 0.626697 |
| 2121 | −5.38249 | 1.458978 | 0.068272 | 0 | 0.037647 | 0 | −0.05982 | −0.16442 | 0.539403 |
| 2122 | −5.38104 | 1.43805 | 0.068269 | 0 | 0.043499 | 0 | −0.06124 | −0.16941 | 0.527702 |
| 2123 | −5.33994 | 1.450455 | 0.067938 | 0 | 0.038042 | 0 | −0.06438 | −0.18655 | 0.571115 |
| 2124 | −5.08915 | 1.476653 | 0.06284 | 0 | −0.00554 | 0 | −0.05799 | 0 | 0.553054 |
| 2125 | −5.12899 | 1.400849 | 0.064834 | 0 | 0.013577 | 0 | −0.05082 | 0 | 0.646123 |
| 2126 | −5.37744 | 1.370868 | 0.069211 | 0 | −0.00232 | 0 | −0.05853 | 0 | 0.63054 |
| 2127 | −5.36591 | 1.354752 | 0.068942 | 0 | −0.00254 | 0 | −0.05917 | 0 | 0.616625 |
| 2128 | −5.36418 | 1.375944 | 0.068846 | 0 | −0.01027 | 0 | −0.06359 | 0 | 0.627382 |
| 2129 | −5.05292 | 1.453829 | 0.062276 | 0 | −0.00806 | 0 | −0.0575 | 0 | 0.538947 |
| 2130 | −5.02622 | 1.477401 | 0.061795 | 0 | −0.0166 | 0 | −0.06129 | 0 | 0.55674 |
| 2131 | −5.24785 | 1.420265 | 0.065988 | 0 | −0.01686 | 0 | −0.0611 | 0 | 0.553095 |
| 2132 | −5.23072 | 1.435697 | 0.065772 | 0 | −0.027 | 0 | −0.06444 | 0 | 0.572012 |
| 2133 | −5.26581 | 1.428642 | 0.066033 | 0 | −0.01996 | 0 | −0.06571 | 0 | 0.541764 |
| 2134 | −5.15607 | 1.483611 | 0.064542 | 0 | 0.027156 | 0 | 0 | −0.15566 | 0.556811 |
| 2135 | −5.21626 | 1.409607 | 0.066943 | 0 | 0.051982 | 0 | 0 | −0.16792 | 0.66156 |
| 2136 | −5.45817 | 1.376931 | 0.071467 | 0 | 0.038034 | 0 | 0 | −0.1836 | 0.665302 |
| 2137 | −5.43575 | 1.357038 | 0.071124 | 0 | 0.046309 | 0 | 0 | −0.18825 | 0.658241 |
| 2138 | −5.41784 | 1.37165 | 0.071088 | 0 | 0.039606 | 0 | 0 | −0.20778 | 0.69148 |
| 2139 | −5.10418 | 1.458195 | 0.063828 | 0 | 0.031253 | 0 | 0 | −0.16089 | 0.550028 |
| 2140 | −5.04686 | 1.476033 | 0.063133 | 0 | 0.023857 | 0 | 0 | −0.17989 | 0.587427 |
| 2141 | −5.29431 | 1.423633 | 0.067661 | 0 | 0.023995 | 0 | 0 | −0.17527 | 0.580899 |
| 2142 | −5.25352 | 1.432896 | 0.067385 | 0 | 0.013894 | 0 | 0 | −0.19463 | 0.622382 |
| 2143 | −5.28344 | 1.424819 | 0.067546 | 0 | 0.026553 | 0 | 0 | −0.19037 | 0.591645 |
| 2144 | −5.09063 | 1.386262 | 0.064699 | 0 | −0.00484 | 0 | 0 | 0 | 0.667415 |
| 2145 | −5.03096 | 1.360023 | 0.063805 | 0 | −0.00783 | 0 | 0 | 0 | 0.656151 |
| 2146 | −5.01786 | 1.387281 | 0.063466 | 0 | −0.01762 | 0 | 0 | 0 | 0.664933 |
| 2147 | −5.30832 | 1.340706 | 0.068608 | 0 | −0.02074 | 0 | 0 | 0 | 0.647439 |
| 2148 | −5.30214 | 1.358266 | 0.068496 | 0 | −0.03275 | 0 | 0 | 0 | 0.657084 |
| 2149 | −5.31036 | 1.351759 | 0.068402 | 0 | −0.02437 | 0 | 0 | 0 | 0.639878 |
| 2150 | −4.98765 | 1.441726 | 0.061824 | 0 | −0.0245 | 0 | 0 | 0 | 0.577155 |
| 2151 | −4.95987 | 1.462085 | 0.061376 | 0 | −0.0365 | 0 | 0 | 0 | 0.59405 |
| 2152 | −4.95589 | 1.449877 | 0.061147 | 0 | −0.03107 | 0 | 0 | 0 | 0.572273 |
| 2153 | −5.1835 | 1.414612 | 0.065436 | 0 | −0.0403 | 0 | 0 | 0 | 0.587187 |
| 2154 | −5.27651 | 1.527271 | 0.065595 | 0 | 0 | −0.09717 | −0.05358 | −0.13213 | 0.504781 |
| 2155 | −5.55856 | 1.423429 | 0.072159 | 0 | 0 | −0.08486 | −0.05075 | −0.15768 | 0.620636 |
| 2156 | −5.41558 | 1.483185 | 0.068725 | 0 | 0 | −0.0947 | −0.05448 | −0.14784 | 0.540663 |
| 2157 | −5.40435 | 1.465288 | 0.068656 | 0 | 0 | −0.11548 | −0.05581 | −0.14896 | 0.53911 |
| 2158 | −5.3713 | 1.475251 | 0.068347 | 0 | 0 | −0.09163 | −0.05947 | −0.16948 | 0.571423 |
| 2159 | −5.12764 | 1.501683 | 0.063559 | 0 | 0 | −0.13626 | −0.05432 | 0 | 0.559474 |
| 2160 | −5.17644 | 1.430811 | 0.065634 | 0 | 0 | −0.1361 | −0.04469 | 0 | 0.650252 |
| 2161 | −5.41953 | 1.394722 | 0.069962 | 0 | 0 | −0.13174 | −0.05411 | 0 | 0.634078 |
| 2162 | −5.40108 | 1.379657 | 0.069733 | 0 | 0 | −0.15462 | −0.05492 | 0 | 0.633025 |
| 2163 | −5.40051 | 1.398234 | 0.069539 | 0 | 0 | −0.13335 | −0.06016 | 0 | 0.632351 |
| 2164 | −5.06839 | 1.480993 | 0.062808 | 0 | 0 | −0.16299 | −0.05349 | 0 | 0.562474 |
| 2165 | −5.05504 | 1.500733 | 0.062406 | 0 | 0 | −0.14187 | −0.05844 | 0 | 0.565267 |
| 2166 | −5.27257 | 1.442101 | 0.066675 | 0 | 0 | −0.15757 | −0.05792 | 0 | 0.572774 |
| 2167 | −5.26047 | 1.454563 | 0.066428 | 0 | 0 | −0.14106 | −0.06241 | 0 | 0.579245 |
| 2168 | −5.28676 | 1.449959 | 0.066656 | 0 | 0 | −0.1543 | −0.06326 | 0 | 0.561232 |
| 2169 | −5.18658 | 1.508047 | 0.06499 | 0 | 0 | −0.10426 | 0 | −0.13862 | 0.560887 |
| 2170 | −5.25809 | 1.439409 | 0.067469 | 0 | 0 | −0.1019 | 0 | −0.14863 | 0.660941 |
| 2171 | −5.49192 | 1.400794 | 0.071896 | 0 | 0 | −0.09443 | 0 | −0.16695 | 0.664072 |
| 2172 | −5.46682 | 1.384329 | 0.071575 | 0 | 0 | −0.11721 | 0 | −0.16731 | 0.663911 |
| 2173 | −5.45118 | 1.396296 | 0.071492 | 0 | 0 | −0.09034 | 0 | −0.1905 | 0.689241 |
| 2174 | −5.11869 | 1.486271 | 0.064126 | 0 | 0 | −0.12958 | 0 | −0.13939 | 0.565959 |
| 2175 | −5.07467 | 1.500697 | 0.063538 | 0 | 0 | −0.10403 | 0 | −0.16263 | 0.591041 |
| 2176 | −5.31532 | 1.446681 | 0.068065 | 0 | 0 | −0.12209 | 0 | −0.15638 | 0.592965 |
| 2177 | −5.27884 | 1.452851 | 0.067778 | 0 | 0 | −0.10045 | 0 | −0.17924 | 0.624825 |
| 2178 | −5.30515 | 1.448957 | 0.067925 | 0 | 0 | −0.11568 | 0 | −0.17156 | 0.601538 |
| 2179 | −5.13541 | 1.413262 | 0.065511 | 0 | 0 | −0.14623 | 0 | 0 | 0.674361 |
| 2180 | −5.06312 | 1.389302 | 0.064569 | 0 | 0 | −0.17555 | 0 | 0 | 0.675956 |
| 2181 | −5.0542 | 1.412424 | 0.064188 | 0 | 0 | −0.15225 | 0 | 0 | 0.673411 |
| 2182 | −5.34226 | 1.362248 | 0.069424 | 0 | 0 | −0.1637 | 0 | 0 | 0.664355 |
| 2183 | −5.33471 | 1.376862 | 0.069204 | 0 | 0 | −0.14694 | 0 | 0 | 0.664386 |
| 2184 | −5.34046 | 1.372648 | 0.069157 | 0 | 0 | −0.15997 | 0 | 0 | 0.656579 |
| 2185 | −5.00458 | 1.465758 | 0.062412 | 0 | 0 | −0.17011 | 0 | 0 | 0.599977 |
| 2186 | −4.98629 | 1.482051 | 0.062004 | 0 | 0 | −0.1519 | 0 | 0 | 0.603221 |
| 2187 | −4.96717 | 1.472835 | 0.061645 | 0 | 0 | −0.16918 | 0 | 0 | 0.594037 |
| 2188 | −5.20391 | 1.432266 | 0.06609 | 0 | 0 | −0.16358 | 0 | 0 | 0.605056 |
| 2189 | −5.18192 | 1.490948 | 0.064713 | 0 | 0 | 0 | −0.05192 | −0.15043 | 0.551619 |
| 2190 | −5.25166 | 1.42223 | 0.067213 | 0 | 0 | 0 | −0.04199 | −0.16016 | 0.654727 |
| 2191 | −5.48147 | 1.386462 | 0.071567 | 0 | 0 | 0 | −0.04797 | −0.17653 | 0.659039 |
| 2192 | −5.46475 | 1.368787 | 0.071278 | 0 | 0 | 0 | −0.0495 | −0.17957 | 0.650102 |
| 2193 | −5.44511 | 1.381781 | 0.071235 | 0 | 0 | 0 | −0.05363 | −0.19985 | 0.685454 |
| 2194 | −5.13608 | 1.466889 | 0.064081 | 0 | 0 | 0 | −0.05221 | −0.15491 | 0.544188 |
| 2195 | −5.07354 | 1.482952 | 0.063325 | 0 | 0 | 0 | −0.05584 | −0.17478 | 0.583491 |
| 2196 | −5.31677 | 1.430573 | 0.067779 | 0 | 0 | 0 | −0.05414 | −0.16972 | 0.57558 |
| 2197 | −5.27016 | 1.437429 | 0.067442 | 0 | 0 | 0 | −0.05737 | −0.19018 | 0.618714 |
| 2198 | −5.3093 | 1.432321 | 0.067705 | 0 | 0 | 0 | −0.05873 | −0.1841 | 0.586676 |
| 2199 | −5.11105 | 1.387646 | 0.064876 | 0 | 0 | 0 | −0.04718 | 0 | 0.662211 |
| 2200 | −5.05421 | 1.360866 | 0.064038 | 0 | 0 | 0 | −0.04643 | 0 | 0.650636 |
| 2201 | −5.03834 | 1.38591 | 0.063661 | 0 | 0 | 0 | −0.05199 | 0 | 0.659781 |
| 2202 | −5.32326 | 1.338738 | 0.068708 | 0 | 0 | 0 | −0.05678 | 0 | 0.640738 |
| 2203 | −5.31358 | 1.353536 | 0.068555 | 0 | 0 | 0 | −0.0617 | 0 | 0.650228 |
| 2204 | −5.32894 | 1.348777 | 0.068556 | 0 | 0 | 0 | −0.0618 | 0 | 0.632736 |
| 2205 | −5.00932 | 1.438351 | 0.062035 | 0 | 0 | 0 | −0.05592 | 0 | 0.569368 |
| 2206 | −4.97738 | 1.455953 | 0.061533 | 0 | 0 | 0 | −0.06055 | 0 | 0.58597 |
| 2207 | −4.97989 | 1.444824 | 0.061402 | 0 | 0 | 0 | −0.06012 | 0 | 0.564146 |
| 2208 | −5.19904 | 1.407831 | 0.065551 | 0 | 0 | 0 | −0.06493 | 0 | 0.577598 |
| 2209 | −5.21199 | 1.403868 | 0.067131 | 0 | 0 | 0 | 0 | −0.16848 | 0.680138 |
| 2210 | −5.14397 | 1.376576 | 0.066187 | 0 | 0 | 0 | 0 | −0.17365 | 0.674322 |
| 2211 | −5.11055 | 1.395004 | 0.06579 | 0 | 0 | 0 | 0 | −0.19657 | 0.70078 |
| 2212 | −5.40793 | 1.350941 | 0.071031 | 0 | 0 | 0 | 0 | −0.18893 | 0.684024 |
| 2213 | −5.38353 | 1.358214 | 0.070994 | 0 | 0 | 0 | 0 | −0.21194 | 0.717639 |
| 2214 | −5.39455 | 1.355063 | 0.070847 | 0 | 0 | 0 | 0 | −0.20403 | 0.696774 |
| 2215 | −5.06765 | 1.451478 | 0.063642 | 0 | 0 | 0 | 0 | −0.162 | 0.584946 |
| 2216 | −5.00565 | 1.462932 | 0.062959 | 0 | 0 | 0 | 0 | −0.18401 | 0.622299 |
| 2217 | −5.00407 | 1.452417 | 0.062725 | 0 | 0 | 0 | 0 | −0.17911 | 0.598954 |
| 2218 | −5.22645 | 1.413634 | 0.067175 | 0 | 0 | 0 | 0 | −0.19454 | 0.632685 |
| 2219 | −5.01851 | 1.347613 | 0.063906 | 0 | 0 | 0 | 0 | 0 | 0.670445 |
| 2220 | −4.99776 | 1.367714 | 0.063519 | 0 | 0 | 0 | 0 | 0 | 0.677883 |
| 2221 | −4.97443 | 1.353789 | 0.06307 | 0 | 0 | 0 | 0 | 0 | 0.666283 |
| 2222 | −5.26837 | 1.330647 | 0.068213 | 0 | 0 | 0 | 0 | 0 | 0.659993 |
| 2223 | −4.91793 | 1.429372 | 0.06101 | 0 | 0 | 0 | 0 | 0 | 0.596405 |
| 2224 | −0.95349 | 1.233873 | 0 | 0.111701 | 0.019203 | −0.09157 | −0.06 | 0 | 0.801939 |
| 2225 | −0.95506 | 1.098116 | 0 | 0.076815 | 0.020371 | −0.07657 | −0.06642 | 0 | 0.683638 |
| 2226 | −0.94876 | 1.167083 | 0 | 0.078045 | 0.002044 | −0.08724 | −0.07252 | 0 | 0.731429 |
| 2227 | −0.9544 | 1.152208 | 0 | 0.089831 | 0.006803 | −0.11853 | −0.06846 | 0 | 0.724674 |
| 2228 | −0.96823 | 1.174569 | 0 | 0.078212 | −0.00515 | −0.09055 | −0.07513 | 0 | 0.724136 |
| 2229 | −0.92323 | 1.231128 | 0 | 0.108439 | 0.038259 | −0.07254 | 0 | −0.10403 | 0.800109 |
| 2230 | −0.91092 | 1.091894 | 0 | 0.075625 | 0.046321 | −0.05598 | 0 | −0.1273 | 0.69677 |
| 2231 | −0.90902 | 1.163403 | 0 | 0.074775 | 0.023565 | −0.06613 | 0 | −0.12319 | 0.738347 |
| 2232 | −0.91013 | 1.145252 | 0 | 0.088072 | 0.032599 | −0.09758 | 0 | −0.1269 | 0.737167 |
| 2233 | −0.90068 | 1.160683 | 0 | 0.078775 | 0.02444 | −0.06466 | 0 | −0.15186 | 0.759254 |
| 2234 | −0.91404 | 1.216369 | 0 | 0.095154 | −0.00319 | −0.10014 | 0 | 0 | 0.819484 |
| 2235 | −0.87857 | 1.110005 | 0 | 0.111259 | 0.023766 | −0.09778 | 0 | 0 | 0.813429 |
| 2236 | −0.9001 | 1.077961 | 0 | 0.06301 | −0.00068 | −0.08806 | 0 | 0 | 0.725232 |
| 2237 | −0.90445 | 1.067678 | 0 | 0.076114 | 0.004801 | −0.11683 | 0 | 0 | 0.726015 |
| 2238 | −0.92118 | 1.090579 | 0 | 0.063589 | −0.007 | −0.08985 | 0 | 0 | 0.720158 |
| 2239 | −0.90545 | 1.196462 | 0 | 0.116031 | 0.001422 | −0.14167 | 0 | 0 | 0.830574 |
| 2240 | −0.92249 | 1.226045 | 0 | 0.100582 | −0.01458 | −0.10861 | 0 | 0 | 0.828673 |
| 2241 | −0.90569 | 1.137667 | 0 | 0.075828 | −0.01364 | −0.12503 | 0 | 0 | 0.761075 |
| 2242 | −0.91243 | 1.157468 | 0 | 0.063176 | −0.02919 | −0.10158 | 0 | 0 | 0.766015 |
| 2243 | −0.93316 | 1.153775 | 0 | 0.074817 | −0.01596 | −0.12118 | 0 | 0 | 0.74786 |
| 2244 | −0.93544 | 1.21709 | 0 | 0.123779 | 0.031425 | 0 | −0.06105 | −0.11116 | 0.791806 |
| 2245 | −0.92139 | 1.081302 | 0 | 0.09131 | 0.042845 | 0 | −0.0659 | −0.13146 | 0.691412 |
| 2246 | −0.92136 | 1.1522 | 0 | 0.092187 | 0.019831 | 0 | −0.07291 | −0.1289 | 0.733147 |
| 2247 | −0.93063 | 1.132208 | 0 | 0.106426 | 0.020846 | 0 | −0.06884 | −0.13516 | 0.719355 |
| 2248 | −0.91193 | 1.148172 | 0 | 0.096894 | 0.020067 | 0 | −0.07578 | −0.15773 | 0.755452 |
| 2249 | −0.93128 | 1.199851 | 0 | 0.112171 | −0.01761 | 0 | −0.06294 | 0 | 0.808971 |
| 2250 | −0.89345 | 1.09504 | 0 | 0.125776 | 0.006993 | 0 | −0.04849 | 0 | 0.803614 |
| 2251 | −0.91553 | 1.064478 | 0 | 0.080845 | −0.01185 | 0 | −0.06861 | 0 | 0.71728 |
| 2252 | −0.92884 | 1.053912 | 0 | 0.094696 | −0.01461 | 0 | −0.06562 | 0 | 0.70582 |
| 2253 | −0.93672 | 1.075967 | 0 | 0.082077 | −0.01944 | 0 | −0.07053 | 0 | 0.711579 |
| 2254 | −0.934 | 1.17688 | 0 | 0.132726 | −0.02382 | 0 | −0.05642 | 0 | 0.803377 |
| 2255 | −0.94065 | 1.207624 | 0 | 0.118228 | −0.03113 | 0 | −0.06475 | 0 | 0.816594 |
| 2256 | −0.933 | 1.122655 | 0 | 0.095772 | −0.03286 | 0 | −0.07162 | 0 | 0.739619 |
| 2257 | −0.93132 | 1.142113 | 0 | 0.083291 | −0.04255 | 0 | −0.07697 | 0 | 0.75592 |
| 2258 | −0.95915 | 1.137339 | 0 | 0.094754 | −0.03477 | 0 | −0.07291 | 0 | 0.726251 |
| 2259 | −0.89467 | 1.199988 | 0 | 0.108154 | 0.007874 | 0 | 0 | −0.11906 | 0.811899 |
| 2260 | −0.85397 | 1.091606 | 0 | 0.126086 | 0.037534 | 0 | 0 | −0.12371 | 0.811124 |
| 2261 | −0.86545 | 1.06021 | 0 | 0.078819 | 0.019339 | 0 | 0 | −0.13933 | 0.734643 |
| 2262 | −0.87234 | 1.045983 | 0 | 0.0944 | 0.021935 | 0 | 0 | −0.14481 | 0.730885 |
| 2263 | −0.86001 | 1.060758 | 0 | 0.083556 | 0.02102 | 0 | 0 | −0.16503 | 0.755722 |
| 2264 | −0.89045 | 1.174064 | 0 | 0.131715 | 0.00709 | 0 | 0 | −0.12805 | 0.815209 |
| 2265 | −0.87475 | 1.197925 | 0 | 0.119495 | 0.004818 | 0 | 0 | −0.15649 | 0.847576 |
| 2266 | −0.88003 | 1.117927 | 0 | 0.093361 | −0.00033 | 0 | 0 | −0.14207 | 0.759526 |
| 2267 | −0.85599 | 1.130893 | 0 | 0.082903 | −0.00663 | 0 | 0 | −0.16609 | 0.796673 |
| 2268 | −0.88625 | 1.12494 | 0 | 0.095222 | 0.003208 | 0 | 0 | −0.16098 | 0.766925 |
| 2269 | −0.86402 | 1.082281 | 0 | 0.111037 | −0.01435 | 0 | 0 | 0 | 0.814569 |
| 2270 | −0.86078 | 1.059842 | 0 | 0.133127 | −0.02147 | 0 | 0 | 0 | 0.819155 |
| 2271 | −0.87174 | 1.089164 | 0 | 0.11682 | −0.02805 | 0 | 0 | 0 | 0.822277 |
| 2272 | −0.88579 | 1.039528 | 0 | 0.080489 | −0.03519 | 0 | 0 | 0 | 0.736377 |
| 2273 | −0.88715 | 1.057794 | 0 | 0.0676 | −0.04484 | 0 | 0 | 0 | 0.746321 |
| 2274 | −0.91171 | 1.057106 | 0 | 0.080503 | −0.03676 | 0 | 0 | 0 | 0.72612 |
| 2275 | −0.90229 | 1.165945 | 0 | 0.116671 | −0.04356 | 0 | 0 | 0 | 0.819111 |
| 2276 | −0.90429 | 1.192584 | 0 | 0.101216 | −0.05572 | 0 | 0 | 0 | 0.832785 |
| 2277 | −0.91718 | 1.181117 | 0 | 0.120071 | −0.04993 | 0 | 0 | 0 | 0.822244 |
| 2278 | −0.91207 | 1.124439 | 0 | 0.079678 | −0.05884 | 0 | 0 | 0 | 0.761676 |
| 2279 | −0.93938 | 1.235429 | 0 | 0.122751 | 0 | −0.06342 | −0.05662 | −0.09859 | 0.794777 |
| 2280 | −0.92988 | 1.099547 | 0 | 0.092202 | 0 | −0.04538 | −0.06122 | −0.12028 | 0.690341 |
| 2281 | −0.92231 | 1.165873 | 0 | 0.090253 | 0 | −0.05925 | −0.06948 | −0.11895 | 0.736691 |
| 2282 | −0.92532 | 1.149336 | 0 | 0.103568 | 0 | −0.08952 | −0.06511 | −0.12134 | 0.732922 |
| 2283 | −0.91385 | 1.16239 | 0 | 0.095079 | 0 | −0.05775 | −0.07277 | −0.14743 | 0.758312 |
| 2284 | −0.92247 | 1.212435 | 0 | 0.105837 | 0 | −0.09766 | −0.06079 | 0 | 0.815103 |
| 2285 | −0.89035 | 1.112791 | 0 | 0.121544 | 0 | −0.08986 | −0.04375 | 0 | 0.807586 |
| 2286 | −0.90879 | 1.075067 | 0 | 0.075219 | 0 | −0.0849 | −0.06668 | 0 | 0.72159 |
| 2287 | −0.91368 | 1.065698 | 0 | 0.087857 | 0 | −0.11293 | −0.06318 | 0 | 0.721144 |
| 2288 | −0.9287 | 1.085495 | 0 | 0.075313 | 0 | −0.08815 | −0.06926 | 0 | 0.716388 |
| 2289 | −0.91366 | 1.193163 | 0 | 0.125288 | 0 | −0.13863 | −0.05309 | 0 | 0.824958 |
| 2290 | −0.92886 | 1.218867 | 0 | 0.110197 | 0 | −0.10856 | −0.06336 | 0 | 0.824403 |
| 2291 | −0.91228 | 1.131512 | 0 | 0.086878 | 0 | −0.12486 | −0.07056 | 0 | 0.757221 |
| 2292 | −0.91706 | 1.147617 | 0 | 0.073887 | 0 | −0.10448 | −0.07745 | 0 | 0.762489 |
| 2293 | −0.93942 | 1.146227 | 0 | 0.085713 | 0 | −0.12162 | −0.07219 | 0 | 0.74366 |
| 2294 | −0.89329 | 1.213632 | 0 | 0.105037 | 0 | −0.07373 | 0 | −0.10812 | 0.816548 |
| 2295 | −0.86051 | 1.111419 | 0 | 0.125974 | 0 | −0.06533 | 0 | −0.11008 | 0.812154 |
| 2296 | −0.86732 | 1.073303 | 0 | 0.077481 | 0 | −0.05869 | 0 | −0.12972 | 0.737088 |
| 2297 | −0.86948 | 1.061956 | 0 | 0.092353 | 0 | −0.08628 | 0 | −0.13141 | 0.740751 |
| 2298 | −0.86328 | 1.074616 | 0 | 0.082393 | 0 | −0.05586 | 0 | −0.15495 | 0.757122 |
| 2299 | −0.88153 | 1.192529 | 0 | 0.127714 | 0 | −0.11305 | 0 | −0.11188 | 0.831554 |
| 2300 | −0.87345 | 1.211721 | 0 | 0.11599 | 0 | −0.0755 | 0 | −0.1452 | 0.851598 |
| 2301 | −0.87018 | 1.129883 | 0 | 0.088387 | 0 | −0.09785 | 0 | −0.13003 | 0.772716 |
| 2302 | −0.85157 | 1.139486 | 0 | 0.07817 | 0 | −0.07118 | 0 | −0.15765 | 0.800541 |
| 2303 | −0.87906 | 1.137881 | 0 | 0.090942 | 0 | −0.09096 | 0 | −0.14919 | 0.778396 |
| 2304 | −0.85556 | 1.095595 | 0 | 0.105312 | 0 | −0.103 | 0 | 0 | 0.821106 |
| 2305 | −0.84209 | 1.075768 | 0 | 0.12623 | 0 | −0.14249 | 0 | 0 | 0.838291 |
| 2306 | −0.86074 | 1.100861 | 0 | 0.108969 | 0 | −0.1126 | 0 | 0 | 0.829793 |
| 2307 | −0.86582 | 1.046478 | 0 | 0.07158 | 0 | −0.12418 | 0 | 0 | 0.75214 |
| 2308 | −0.87262 | 1.062136 | 0 | 0.057869 | 0 | −0.10521 | 0 | 0 | 0.752651 |
| 2309 | −0.89324 | 1.064319 | 0 | 0.07123 | 0 | −0.12002 | 0 | 0 | 0.740757 |
| 2310 | −0.87863 | 1.177526 | 0 | 0.107341 | 0 | −0.14578 | 0 | 0 | 0.839023 |
| 2311 | −0.88734 | 1.199147 | 0 | 0.090522 | 0 | −0.1218 | 0 | 0 | 0.840246 |
| 2312 | −0.89361 | 1.192243 | 0 | 0.110085 | 0 | −0.14683 | 0 | 0 | 0.841933 |
| 2313 | −0.88807 | 1.128184 | 0 | 0.067831 | 0 | −0.1331 | 0 | 0 | 0.776809 |
| 2314 | −0.90515 | 1.199582 | 0 | 0.120042 | 0 | 0 | −0.06101 | −0.11633 | 0.808457 |
| 2315 | −0.86903 | 1.098883 | 0 | 0.137111 | 0 | 0 | −0.04298 | −0.11714 | 0.805225 |
| 2316 | −0.87795 | 1.06309 | 0 | 0.092657 | 0 | 0 | −0.06441 | −0.13456 | 0.731488 |
| 2317 | −0.88493 | 1.049042 | 0 | 0.107675 | 0 | 0 | −0.06138 | −0.1395 | 0.726404 |
| 2318 | −0.87287 | 1.063383 | 0 | 0.097891 | 0 | 0 | −0.06673 | −0.15986 | 0.752485 |
| 2319 | −0.89989 | 1.172927 | 0 | 0.141944 | 0 | 0 | −0.05464 | −0.12554 | 0.810457 |
| 2320 | −0.88434 | 1.195945 | 0 | 0.131393 | 0 | 0 | −0.06329 | −0.15423 | 0.844742 |
| 2321 | −0.88867 | 1.115753 | 0 | 0.106067 | 0 | 0 | −0.07026 | −0.14009 | 0.757149 |
| 2322 | −0.86342 | 1.127026 | 0 | 0.096413 | 0 | 0 | −0.07682 | −0.16506 | 0.796095 |
| 2323 | −0.89544 | 1.122594 | 0 | 0.108492 | 0 | 0 | −0.07136 | −0.15856 | 0.764446 |
| 2324 | −0.8696 | 1.076791 | 0 | 0.118641 | 0 | 0 | −0.05079 | 0 | 0.809917 |
| 2325 | −0.86457 | 1.052503 | 0 | 0.138194 | 0 | 0 | −0.04561 | 0 | 0.813803 |
| 2326 | −0.87541 | 1.079817 | 0 | 0.122909 | 0 | 0 | −0.05301 | 0 | 0.816995 |
| 2327 | −0.88947 | 1.028511 | 0 | 0.088954 | 0 | 0 | −0.06996 | 0 | 0.731052 |
| 2328 | −0.89011 | 1.044171 | 0 | 0.076208 | 0 | 0 | −0.07498 | 0 | 0.740391 |
| 2329 | −0.91543 | 1.04514 | 0 | 0.088553 | 0 | 0 | −0.07056 | 0 | 0.71974 |
| 2330 | −0.90559 | 1.152609 | 0 | 0.122697 | 0 | 0 | −0.06188 | 0 | 0.811463 |
| 2331 | −0.90672 | 1.176257 | 0 | 0.107896 | 0 | 0 | −0.07002 | 0 | 0.824818 |
| 2332 | −0.91938 | 1.165592 | 0 | 0.12544 | 0 | 0 | −0.06329 | 0 | 0.813995 |
| 2333 | −0.91478 | 1.107394 | 0 | 0.087124 | 0 | 0 | −0.07892 | 0 | 0.752872 |
| 2334 | −0.83313 | 1.080574 | 0 | 0.121903 | 0 | 0 | 0 | −0.12677 | 0.819705 |
| 2335 | −0.82157 | 1.055029 | 0 | 0.145663 | 0 | 0 | 0 | −0.13632 | 0.832246 |
| 2336 | −0.81438 | 1.075374 | 0 | 0.132823 | 0 | 0 | 0 | −0.16242 | 0.850633 |
| 2337 | −0.83541 | 1.028905 | 0 | 0.092752 | 0 | 0 | 0 | −0.14926 | 0.759762 |
| 2338 | −0.81493 | 1.037582 | 0 | 0.0822 | 0 | 0 | 0 | −0.17219 | 0.789434 |
| 2339 | −0.84411 | 1.038802 | 0 | 0.09594 | 0 | 0 | 0 | −0.16538 | 0.76758 |
| 2340 | −0.86304 | 1.157985 | 0 | 0.124764 | 0 | 0 | 0 | −0.13435 | 0.827642 |
| 2341 | −0.84198 | 1.175783 | 0 | 0.112451 | 0 | 0 | 0 | −0.16408 | 0.860777 |
| 2342 | −0.85681 | 1.164279 | 0 | 0.132451 | 0 | 0 | 0 | −0.16026 | 0.850823 |
| 2343 | −0.84156 | 1.103859 | 0 | 0.092004 | 0 | 0 | 0 | −0.16987 | 0.800393 |
| 2344 | −0.83824 | 1.04086 | 0 | 0.121696 | 0 | 0 | 0 | 0 | 0.822245 |
| 2345 | −0.84297 | 1.063182 | 0 | 0.105257 | 0 | 0 | 0 | 0 | 0.826988 |
| 2346 | −0.84986 | 1.051692 | 0 | 0.12418 | 0 | 0 | 0 | 0 | 0.824926 |
| 2347 | −0.86966 | 1.026261 | 0 | 0.070396 | 0 | 0 | 0 | 0 | 0.746865 |
| 2348 | −0.88518 | 1.150839 | 0 | 0.105561 | 0 | 0 | 0 | 0 | 0.826759 |
| 2349 | −0.95207 | 1.241254 | 0 | 0 | 0.058158 | −0.07993 | −0.04736 | −0.10002 | 0.773465 |
| 2350 | −0.9316 | 1.100367 | 0 | 0 | 0.063866 | −0.06189 | −0.05522 | −0.12308 | 0.681503 |
| 2351 | −0.92958 | 1.170847 | 0 | 0 | 0.042533 | −0.07174 | −0.06164 | −0.11936 | 0.725297 |
| 2352 | −0.93432 | 1.15462 | 0 | 0 | 0.051674 | −0.10372 | −0.05668 | −0.12257 | 0.717224 |
| 2353 | −0.92172 | 1.167964 | 0 | 0 | 0.043521 | −0.07093 | −0.06448 | −0.14709 | 0.745692 |
| 2354 | −0.95257 | 1.160375 | 0 | 0 | 0.003438 | −0.12566 | −0.06193 | 0 | 0.733113 |
| 2355 | −0.93832 | 1.225601 | 0 | 0 | 0.016819 | −0.10556 | −0.04933 | 0 | 0.798993 |
| 2356 | −0.90352 | 1.123522 | 0 | 0 | 0.042654 | −0.10452 | −0.03412 | 0 | 0.789637 |
| 2357 | −0.9168 | 1.084173 | 0 | 0 | 0.017089 | −0.09197 | −0.05843 | 0 | 0.714397 |
| 2358 | −0.92401 | 1.076269 | 0 | 0 | 0.023084 | −0.1214 | −0.0545 | 0 | 0.710001 |
| 2359 | −0.93753 | 1.096123 | 0 | 0 | 0.010692 | −0.09401 | −0.06079 | 0 | 0.70921 |
| 2360 | −0.93408 | 1.209195 | 0 | 0 | 0.02214 | −0.14718 | −0.04112 | 0 | 0.80165 |
| 2361 | −0.94707 | 1.235556 | 0 | 0 | 0.006069 | −0.1142 | −0.0512 | 0 | 0.807479 |
| 2362 | −0.92567 | 1.145376 | 0 | 0 | 0.005605 | −0.12943 | −0.05978 | 0 | 0.746337 |
| 2363 | −0.92882 | 1.162442 | 0 | 0 | −0.01036 | −0.10557 | −0.06666 | 0 | 0.757141 |
| 2364 | −0.89022 | 1.145067 | 0 | 0 | 0.034223 | −0.10146 | 0 | −0.14782 | 0.76428 |
| 2365 | −0.90765 | 1.221615 | 0 | 0 | 0.036197 | −0.08523 | 0 | −0.10712 | 0.799322 |
| 2366 | −0.87124 | 1.11661 | 0 | 0 | 0.067434 | −0.08393 | 0 | −0.1111 | 0.793108 |
| 2367 | −0.87234 | 1.076882 | 0 | 0 | 0.042186 | −0.07023 | 0 | −0.13008 | 0.728761 |
| 2368 | −0.8761 | 1.065792 | 0 | 0 | 0.052964 | −0.10022 | 0 | −0.1327 | 0.728412 |
| 2369 | −0.86865 | 1.078327 | 0 | 0 | 0.044376 | −0.06852 | 0 | −0.15504 | 0.747635 |
| 2370 | −0.90054 | 1.201944 | 0 | 0 | 0.045786 | −0.1262 | 0 | −0.11108 | 0.805818 |
| 2371 | −0.89093 | 1.221822 | 0 | 0 | 0.034832 | −0.08745 | 0 | −0.14225 | 0.831023 |
| 2372 | −0.88155 | 1.137569 | 0 | 0 | 0.03089 | −0.1075 | 0 | −0.12888 | 0.759637 |
| 2373 | −0.86227 | 1.148198 | 0 | 0 | 0.018129 | −0.07866 | 0 | −0.15482 | 0.7912 |
| 2374 | −0.87066 | 1.107723 | 0 | 0 | 0.024243 | −0.11205 | 0 | 0 | 0.805486 |
| 2375 | −0.86171 | 1.091256 | 0 | 0 | 0.02927 | −0.15246 | 0 | 0 | 0.815882 |
| 2376 | −0.8781 | 1.116364 | 0 | 0 | 0.012884 | −0.11962 | 0 | 0 | 0.813386 |
| 2377 | −0.87764 | 1.05863 | 0 | 0 | 0.003502 | −0.12743 | 0 | 0 | 0.743906 |
| 2378 | −0.88329 | 1.075022 | 0 | 0 | −0.01264 | −0.1048 | 0 | 0 | 0.74898 |
| 2379 | −0.90504 | 1.076607 | 0 | 0 | 0.001578 | −0.12297 | 0 | 0 | 0.732424 |
| 2380 | −0.89979 | 1.194569 | 0 | 0 | 0.003356 | −0.14936 | 0 | 0 | 0.820564 |
| 2381 | −0.90645 | 1.217478 | 0 | 0 | −0.01658 | −0.1212 | 0 | 0 | 0.829251 |
| 2382 | −0.91609 | 1.210984 | 0 | 0 | −0.00234 | −0.14918 | 0 | 0 | 0.822635 |
| 2383 | −0.90241 | 1.144531 | 0 | 0 | −0.01996 | −0.13125 | 0 | 0 | 0.771329 |
| 2384 | −0.91156 | 1.134288 | 0 | 0 | 0.022714 | 0 | −0.05972 | −0.15591 | 0.74707 |
| 2385 | −0.88304 | 1.106062 | 0 | 0 | 0.055913 | 0 | −0.03304 | −0.11894 | 0.784306 |
| 2386 | −0.92221 | 1.209627 | 0 | 0 | 0.027892 | 0 | −0.04876 | −0.11534 | 0.789112 |
| 2387 | −0.88568 | 1.068615 | 0 | 0 | 0.037419 | 0 | −0.05593 | −0.13525 | 0.721551 |
| 2388 | −0.89696 | 1.057219 | 0 | 0 | 0.040299 | 0 | −0.05165 | −0.14011 | 0.710645 |
| 2389 | −0.88128 | 1.069394 | 0 | 0 | 0.038871 | 0 | −0.05799 | −0.1601 | 0.741093 |
| 2390 | −0.92404 | 1.187041 | 0 | 0 | 0.027837 | 0 | −0.04093 | −0.12339 | 0.781541 |
| 2391 | −0.90498 | 1.209002 | 0 | 0 | 0.025181 | 0 | −0.05018 | −0.15089 | 0.821845 |
| 2392 | −0.90479 | 1.127694 | 0 | 0 | 0.019265 | 0 | −0.05809 | −0.13781 | 0.740976 |
| 2393 | −0.8772 | 1.138674 | 0 | 0 | 0.012721 | 0 | −0.06516 | −0.16159 | 0.784509 |
| 2394 | −0.88854 | 1.094748 | 0 | 0 | 0.004988 | 0 | −0.03677 | 0 | 0.792992 |
| 2395 | −0.88994 | 1.076903 | 0 | 0 | −0.00103 | 0 | −0.03043 | 0 | 0.789885 |
| 2396 | −0.89671 | 1.102426 | 0 | 0 | −0.00858 | 0 | −0.03832 | 0 | 0.799708 |
| 2397 | −0.90568 | 1.048301 | 0 | 0 | −0.01601 | 0 | −0.05639 | 0 | 0.721872 |
| 2398 | −0.9036 | 1.063618 | 0 | 0 | −0.02634 | 0 | −0.06185 | 0 | 0.736697 |
| 2399 | −0.93151 | 1.065176 | 0 | 0 | −0.01761 | 0 | −0.05774 | 0 | 0.710681 |
| 2400 | −0.9306 | 1.177783 | 0 | 0 | −0.02217 | 0 | −0.0444 | 0 | 0.792605 |
| 2401 | −0.92838 | 1.201677 | 0 | 0 | −0.03483 | 0 | −0.05268 | 0 | 0.813887 |
| 2402 | −0.9458 | 1.192981 | 0 | 0 | −0.02789 | 0 | −0.04601 | 0 | 0.794602 |
| 2403 | −0.93209 | 1.131599 | 0 | 0 | −0.03872 | 0 | −0.06327 | 0 | 0.747998 |
| 2404 | −0.84985 | 1.090407 | 0 | 0 | 0.036413 | 0 | 0 | −0.12569 | 0.800885 |
| 2405 | −0.84564 | 1.06959 | 0 | 0 | 0.036755 | 0 | 0 | −0.1341 | 0.804137 |
| 2406 | −0.83384 | 1.08766 | 0 | 0 | 0.034432 | 0 | 0 | −0.15956 | 0.828985 |
| 2407 | −0.8496 | 1.039725 | 0 | 0 | 0.020245 | 0 | 0 | −0.14703 | 0.746822 |
| 2408 | −0.82687 | 1.04778 | 0 | 0 | 0.013329 | 0 | 0 | −0.16906 | 0.780399 |
| 2409 | −0.85818 | 1.048943 | 0 | 0 | 0.024564 | 0 | 0 | −0.16319 | 0.753199 |
| 2410 | −0.88802 | 1.173849 | 0 | 0 | 0.009673 | 0 | 0 | −0.12962 | 0.804314 |
| 2411 | −0.86419 | 1.191823 | 0 | 0 | 0.001309 | 0 | 0 | −0.15784 | 0.843958 |
| 2412 | −0.88408 | 1.181484 | 0 | 0 | 0.010337 | 0 | 0 | −0.15405 | 0.824431 |
| 2413 | −0.85954 | 1.118857 | 0 | 0 | −0.00089 | 0 | 0 | −0.16421 | 0.788112 |
| 2414 | −0.86486 | 1.066186 | 0 | 0 | −0.01625 | 0 | 0 | 0 | 0.802286 |
| 2415 | −0.86622 | 1.087968 | 0 | 0 | −0.02886 | 0 | 0 | 0 | 0.814108 |
| 2416 | −0.87733 | 1.079138 | 0 | 0 | −0.02321 | 0 | 0 | 0 | 0.804963 |
| 2417 | −0.8864 | 1.048787 | 0 | 0 | −0.04081 | 0 | 0 | 0 | 0.743781 |
| 2418 | −0.91163 | 1.180027 | 0 | 0 | −0.04895 | 0 | 0 | 0 | 0.815183 |
| 2419 | −0.90637 | 1.152202 | 0 | 0 | 0 | −0.09434 | −0.05649 | −0.14136 | 0.760757 |
| 2420 | −0.89226 | 1.131861 | 0 | 0 | 0 | −0.07132 | −0.0261 | −0.10234 | 0.785731 |
| 2421 | −0.92309 | 1.228495 | 0 | 0 | 0 | −0.0776 | −0.04424 | −0.10158 | 0.795271 |
| 2422 | −0.89012 | 1.086417 | 0 | 0 | 0 | −0.06142 | −0.05084 | −0.12315 | 0.72447 |
| 2423 | −0.89642 | 1.078146 | 0 | 0 | 0 | −0.08973 | −0.04667 | −0.124 | 0.721545 |
| 2424 | −0.88742 | 1.08838 | 0 | 0 | 0 | −0.05934 | −0.05349 | −0.14728 | 0.742936 |
| 2425 | −0.91708 | 1.211338 | 0 | 0 | 0 | −0.11717 | −0.03605 | −0.10425 | 0.800421 |
| 2426 | −0.90624 | 1.228456 | 0 | 0 | 0 | −0.08013 | −0.04642 | −0.13653 | 0.827457 |
| 2427 | −0.89652 | 1.144212 | 0 | 0 | 0 | −0.10077 | −0.05451 | −0.12313 | 0.756518 |
| 2428 | −0.87475 | 1.151753 | 0 | 0 | 0 | −0.07386 | −0.06258 | −0.15056 | 0.790405 |
| 2429 | −0.88196 | 1.112955 | 0 | 0 | 0 | −0.10517 | −0.03229 | 0 | 0.800543 |
| 2430 | −0.87255 | 1.097941 | 0 | 0 | 0 | −0.14484 | −0.02525 | 0 | 0.811305 |
| 2431 | −0.88716 | 1.11876 | 0 | 0 | 0 | −0.11504 | −0.03502 | 0 | 0.809252 |
| 2432 | −0.88681 | 1.058784 | 0 | 0 | 0 | −0.12457 | −0.05484 | 0 | 0.739729 |
| 2433 | −0.89024 | 1.071076 | 0 | 0 | 0 | −0.10507 | −0.0619 | 0 | 0.745178 |
| 2434 | −0.91411 | 1.07593 | 0 | 0 | 0 | −0.12065 | −0.0566 | 0 | 0.727801 |
| 2435 | −0.90808 | 1.194153 | 0 | 0 | 0 | −0.14684 | −0.04218 | 0 | 0.815601 |
| 2436 | −0.91259 | 1.212283 | 0 | 0 | 0 | −0.12252 | −0.05268 | 0 | 0.824353 |
| 2437 | −0.92347 | 1.208876 | 0 | 0 | 0 | −0.14792 | −0.04445 | 0 | 0.817668 |
| 2438 | −0.90861 | 1.138639 | 0 | 0 | 0 | −0.13323 | −0.06421 | 0 | 0.766702 |
| 2439 | −0.85419 | 1.111329 | 0 | 0 | 0 | −0.08009 | 0 | −0.11048 | 0.805015 |
| 2440 | −0.84322 | 1.094848 | 0 | 0 | 0 | −0.11809 | 0 | −0.11343 | 0.818109 |
| 2441 | −0.83871 | 1.109392 | 0 | 0 | 0 | −0.08181 | 0 | −0.14351 | 0.832574 |
| 2442 | −0.84368 | 1.054857 | 0 | 0 | 0 | −0.09745 | 0 | −0.13286 | 0.759204 |
| 2443 | −0.82554 | 1.060198 | 0 | 0 | 0 | −0.07236 | 0 | −0.15836 | 0.785246 |
| 2444 | −0.85563 | 1.065834 | 0 | 0 | 0 | −0.09034 | 0 | −0.14887 | 0.763345 |
| 2445 | −0.87762 | 1.192774 | 0 | 0 | 0 | −0.12079 | 0 | −0.11237 | 0.821968 |
| 2446 | −0.8598 | 1.205479 | 0 | 0 | 0 | −0.089 | 0 | −0.14545 | 0.850273 |
| 2447 | −0.87605 | 1.201224 | 0 | 0 | 0 | −0.11652 | 0 | −0.13709 | 0.839939 |
| 2448 | −0.84897 | 1.130348 | 0 | 0 | 0 | −0.10175 | 0 | −0.15194 | 0.801155 |
| 2449 | −0.84431 | 1.082841 | 0 | 0 | 0 | −0.1506 | 0 | 0 | 0.823761 |
| 2450 | −0.8512 | 1.099423 | 0 | 0 | 0 | −0.12673 | 0 | 0 | 0.825373 |
| 2451 | −0.85709 | 1.094993 | 0 | 0 | 0 | −0.15089 | 0 | 0 | 0.825853 |
| 2452 | −0.86349 | 1.053592 | 0 | 0 | 0 | −0.13156 | 0 | 0 | 0.760685 |
| 2453 | −0.88537 | 1.19048 | 0 | 0 | 0 | −0.15454 | 0 | 0 | 0.836075 |
| 2454 | −0.86361 | 1.099474 | 0 | 0 | 0 | 0 | −0.02976 | −0.11936 | 0.796626 |
| 2455 | −0.85804 | 1.078921 | 0 | 0 | 0 | 0 | −0.02374 | −0.12755 | 0.80025 |
| 2456 | −0.84747 | 1.096582 | 0 | 0 | 0 | 0 | −0.03128 | −0.15299 | 0.824946 |
| 2457 | −0.86176 | 1.044749 | 0 | 0 | 0 | 0 | −0.05051 | −0.14178 | 0.74338 |
| 2458 | −0.8382 | 1.051044 | 0 | 0 | 0 | 0 | −0.05663 | −0.16463 | 0.778012 |
| 2459 | −0.87165 | 1.054926 | 0 | 0 | 0 | 0 | −0.05144 | −0.15709 | 0.749068 |
| 2460 | −0.89742 | 1.175638 | 0 | 0 | 0 | 0 | −0.04142 | −0.12667 | 0.800316 |
| 2461 | −0.87303 | 1.191576 | 0 | 0 | 0 | 0 | −0.05019 | −0.1558 | 0.840788 |
| 2462 | −0.89365 | 1.183214 | 0 | 0 | 0 | 0 | −0.04261 | −0.15092 | 0.820557 |
| 2463 | −0.86845 | 1.118285 | 0 | 0 | 0 | 0 | −0.06065 | −0.16201 | 0.785496 |
| 2464 | −0.8684 | 1.061673 | 0 | 0 | 0 | 0 | −0.03325 | 0 | 0.797836 |
| 2465 | −0.8691 | 1.080046 | 0 | 0 | 0 | 0 | −0.0415 | 0 | 0.808712 |
| 2466 | −0.88002 | 1.072655 | 0 | 0 | 0 | 0 | −0.03513 | 0 | 0.799987 |
| 2467 | −0.89 | 1.037841 | 0 | 0 | 0 | 0 | −0.0626 | 0 | 0.736806 |
| 2468 | −0.914 | 1.166655 | 0 | 0 | 0 | 0 | −0.05176 | 0 | 0.806183 |
| 2469 | −0.8291 | 1.06435 | 0 | 0 | 0 | 0 | 0 | −0.13471 | 0.81403 |
| 2470 | −0.8108 | 1.076307 | 0 | 0 | 0 | 0 | 0 | −0.16228 | 0.841272 |
| 2471 | −0.82461 | 1.069457 | 0 | 0 | 0 | 0 | 0 | −0.1578 | 0.832141 |
| 2472 | −0.8188 | 1.031615 | 0 | 0 | 0 | 0 | 0 | −0.16891 | 0.78457 |
| 2473 | −0.85378 | 1.16574 | 0 | 0 | 0 | 0 | 0 | −0.16034 | 0.841877 |
| 2474 | −0.85124 | 1.057763 | 0 | 0 | 0 | 0 | 0 | 0 | 0.812071 |
| 2475 | −0.17624 | 0 | 0 | 0 | 0 | −0.04295 | −0.02922 | −0.11378 | 0.886334 |
| 2476 | −0.17435 | 0 | 0 | 0 | 0 | −0.08512 | −0.02284 | −0.11776 | 0.898877 |
| 2477 | −0.16565 | 0 | 0 | 0 | 0 | −0.04369 | −0.03091 | −0.15475 | 0.908318 |
| 2478 | −0.17234 | 0 | 0 | 0 | 0 | −0.07375 | −0.04921 | −0.13647 | 0.890454 |
| 2479 | −0.15573 | 0 | 0 | 0 | 0 | −0.04337 | −0.05565 | −0.16839 | 0.909418 |
| 2480 | −0.18116 | 0 | 0 | 0 | 0 | −0.06386 | −0.05143 | −0.15799 | 0.891977 |
| 2481 | −0.17568 | 0 | 0 | 0 | 0 | −0.08313 | −0.04237 | −0.11527 | 0.882152 |
| 2482 | −0.15626 | 0 | 0 | 0 | 0 | −0.04806 | −0.05111 | −0.15521 | 0.902309 |
| 2483 | −0.17198 | 0 | 0 | 0 | 0 | −0.07695 | −0.04512 | −0.14747 | 0.896615 |
| 2484 | −0.15589 | 0 | 0 | 0 | 0 | −0.07303 | −0.05996 | −0.16099 | 0.905102 |
| 2485 | −0.18171 | 0 | 0 | 0 | 0 | −0.1165 | −0.02964 | 0 | 0.906084 |
| 2486 | −0.18502 | 0 | 0 | 0 | 0 | −0.0889 | −0.03879 | 0 | 0.901254 |
| 2487 | −0.19211 | 0 | 0 | 0 | 0 | −0.11492 | −0.03213 | 0 | 0.90253 |
| 2488 | −0.19819 | 0 | 0 | 0 | 0 | −0.10453 | −0.06109 | 0 | 0.885997 |
| 2489 | −0.18734 | 0 | 0 | 0 | 0 | −0.11423 | −0.05163 | 0 | 0.893858 |
| 2490 | −0.15051 | 0 | 0 | 0 | 0 | −0.09133 | 0 | −0.12432 | 0.91486 |
| 2491 | −0.13571 | 0 | 0 | 0 | 0 | −0.05618 | 0 | −0.16293 | 0.928804 |
| 2492 | −0.14784 | 0 | 0 | 0 | 0 | −0.08394 | 0 | −0.15381 | 0.926726 |
| 2493 | −0.13909 | 0 | 0 | 0 | 0 | −0.07539 | 0 | −0.1697 | 0.924259 |
| 2494 | −0.13847 | 0 | 0 | 0 | 0 | −0.08375 | 0 | −0.15543 | 0.919632 |
| 2495 | −0.16834 | 0 | 0 | 0 | 0 | −0.12373 | 0 | 0 | 0.917565 |
| 2496 | −0.17101 | 0 | 0 | 0 | 0 | 0 | −0.02636 | −0.1338 | 0.898657 |
| 2497 | −0.14986 | 0 | 0 | 0 | 0 | 0 | −0.03283 | −0.16832 | 0.920647 |
| 2498 | −0.16674 | 0 | 0 | 0 | 0 | 0 | −0.02759 | −0.16261 | 0.912643 |
| 2499 | −0.16034 | 0 | 0 | 0 | 0 | 0 | −0.05421 | −0.17493 | 0.910091 |
| 2500 | −0.16197 | 0 | 0 | 0 | 0 | 0 | −0.04747 | −0.16376 | 0.903705 |
| 2501 | −0.19985 | 0 | 0 | 0 | 0 | 0 | −0.03733 | 0 | 0.893188 |
| 2502 | −0.1422 | 0 | 0 | 0 | 0 | 0 | 0 | −0.17122 | 0.928309 |
| 2503 | −3.61501 | 0 | 0.053728 | 0.226878 | 0.077014 | −0.07771 | 0 | 0 | 0.636393 |
| 2504 | −4.02974 | 0 | 0.061921 | 0.223011 | 0.075357 | −0.07789 | 0 | 0 | 0.79335 |
| 2505 | −3.80102 | 0 | 0.057411 | 0.20518 | 0.060257 | −0.08238 | 0 | 0 | 0.684965 |
| 2506 | −3.83066 | 0 | 0.057995 | 0.222642 | 0.067273 | −0.11394 | 0 | 0 | 0.710645 |
| 2507 | −3.82792 | 0 | 0.057744 | 0.209247 | 0.052598 | −0.08366 | 0 | 0 | 0.683139 |
| 2508 | −3.65415 | 0 | 0.053925 | 0.247472 | 0.073059 | 0 | −0.09348 | 0 | 0.62599 |
| 2509 | −4.05967 | 0 | 0.061984 | 0.24269 | 0.070321 | 0 | −0.08966 | 0 | 0.783882 |
| 2510 | −3.82985 | 0 | 0.057429 | 0.224951 | 0.055336 | 0 | −0.09262 | 0 | 0.673632 |
| 2511 | −3.87906 | 0 | 0.058142 | 0.244188 | 0.054028 | 0 | −0.09444 | 0 | 0.684472 |
| 2512 | −3.86377 | 0 | 0.057852 | 0.231996 | 0.047398 | 0 | −0.09916 | 0 | 0.672128 |
| 2513 | −3.69365 | 0 | 0.055264 | 0.240181 | 0.09934 | 0 | 0 | −0.15825 | 0.61905 |
| 2514 | −4.11324 | 0 | 0.063909 | 0.244612 | 0.104719 | 0 | 0 | −0.18786 | 0.811525 |
| 2515 | −3.86294 | 0 | 0.058857 | 0.221679 | 0.083648 | 0 | 0 | −0.17333 | 0.684505 |
| 2516 | −3.89951 | 0 | 0.0595 | 0.241756 | 0.090239 | 0 | 0 | −0.18297 | 0.708304 |
| 2517 | −3.86579 | 0 | 0.059263 | 0.234012 | 0.086779 | 0 | 0 | −0.21082 | 0.725847 |
| 2518 | −3.5485 | 0 | 0.052937 | 0.222708 | 0.045306 | 0 | 0 | 0 | 0.67203 |
| 2519 | −3.67049 | 0 | 0.055794 | 0.25561 | 0.06369 | 0 | 0 | 0 | 0.791156 |
| 2520 | −3.97472 | 0 | 0.061193 | 0.218199 | 0.040242 | 0 | 0 | 0 | 0.806868 |
| 2521 | −4.00947 | 0 | 0.06157 | 0.236717 | 0.03965 | 0 | 0 | 0 | 0.810735 |
| 2522 | −3.98748 | 0 | 0.061219 | 0.222687 | 0.031825 | 0 | 0 | 0 | 0.800123 |
| 2523 | −3.53373 | 0 | 0.052726 | 0.245931 | 0.04267 | 0 | 0 | 0 | 0.694555 |
| 2524 | −3.51382 | 0 | 0.052326 | 0.230717 | 0.033594 | 0 | 0 | 0 | 0.674877 |
| 2525 | −3.76806 | 0 | 0.057011 | 0.218736 | 0.02795 | 0 | 0 | 0 | 0.721871 |
| 2526 | −3.75014 | 0 | 0.056753 | 0.206227 | 0.016931 | 0 | 0 | 0 | 0.71424 |
| 2527 | −3.80931 | 0 | 0.057462 | 0.222391 | 0.025077 | 0 | 0 | 0 | 0.708837 |
| 2528 | −3.65527 | 0 | 0.054074 | 0.25 | 0 | −0.05872 | −0.08433 | 0 | 0.630292 |
| 2529 | −4.06462 | 0 | 0.062183 | 0.24523 | 0 | −0.05857 | −0.08099 | 0 | 0.787375 |
| 2530 | −3.82956 | 0 | 0.057605 | 0.226701 | 0 | −0.06673 | −0.08503 | 0 | 0.682423 |
| 2531 | −3.86496 | 0 | 0.05826 | 0.245612 | 0 | −0.09612 | −0.08641 | 0 | 0.705413 |
| 2532 | −3.85743 | 0 | 0.057965 | 0.232993 | 0 | −0.06906 | −0.09255 | 0 | 0.683467 |
| 2533 | −3.6964 | 0 | 0.055279 | 0.246118 | 0 | −0.02904 | 0 | −0.14205 | 0.621844 |
| 2534 | −4.12026 | 0 | 0.063952 | 0.251375 | 0 | −0.02378 | 0 | −0.17251 | 0.811404 |
| 2535 | −3.8636 | 0 | 0.058889 | 0.226434 | 0 | −0.03423 | 0 | −0.15893 | 0.691235 |
| 2536 | −3.88825 | 0 | 0.059431 | 0.246148 | 0 | −0.06348 | 0 | −0.16274 | 0.720812 |
| 2537 | −3.86518 | 0 | 0.059257 | 0.239186 | 0 | −0.02931 | 0 | −0.19552 | 0.730997 |
| 2538 | −3.55148 | 0 | 0.053158 | 0.225459 | 0 | −0.07354 | 0 | 0 | 0.68028 |
| 2539 | −3.6816 | 0 | 0.056101 | 0.259263 | 0 | −0.07432 | 0 | 0 | 0.794985 |
| 2540 | −3.98258 | 0 | 0.061486 | 0.220393 | 0 | −0.07795 | 0 | 0 | 0.813982 |
| 2541 | −4.0088 | 0 | 0.06188 | 0.238841 | 0 | −0.10824 | 0 | 0 | 0.828153 |
| 2542 | −3.9905 | 0 | 0.061464 | 0.223848 | 0 | −0.07895 | 0 | 0 | 0.808699 |
| 2543 | −3.51779 | 0 | 0.052824 | 0.249229 | 0 | −0.10824 | 0 | 0 | 0.716122 |
| 2544 | −3.50869 | 0 | 0.05246 | 0.232374 | 0 | −0.07945 | 0 | 0 | 0.685564 |
| 2545 | −3.75879 | 0 | 0.057224 | 0.219877 | 0 | −0.10931 | 0 | 0 | 0.743445 |
| 2546 | −3.74696 | 0 | 0.056953 | 0.206164 | 0 | −0.08693 | 0 | 0 | 0.726744 |
| 2547 | −3.79753 | 0 | 0.057636 | 0.223339 | 0 | −0.10406 | 0 | 0 | 0.730802 |
| 2548 | −3.7312 | 0 | 0.055568 | 0.26352 | 0 | 0 | −0.08114 | −0.14355 | 0.616085 |
| 2549 | −4.14766 | 0 | 0.064142 | 0.266754 | 0 | 0 | −0.07538 | −0.17242 | 0.805645 |
| 2550 | −3.88943 | 0 | 0.059023 | 0.243281 | 0 | 0 | −0.08015 | −0.16033 | 0.684933 |
| 2551 | −3.93061 | 0 | 0.059722 | 0.264964 | 0 | 0 | −0.08305 | −0.16803 | 0.706353 |
| 2552 | −3.89565 | 0 | 0.059475 | 0.258451 | 0 | 0 | −0.08804 | −0.19682 | 0.727732 |
| 2553 | −3.58085 | 0 | 0.053231 | 0.242805 | 0 | 0 | −0.08235 | 0 | 0.669061 |
| 2554 | −3.70898 | 0 | 0.056177 | 0.273899 | 0 | 0 | −0.07019 | 0 | 0.785059 |
| 2555 | −4.00082 | 0 | 0.061389 | 0.23701 | 0 | 0 | −0.07917 | 0 | 0.803627 |
| 2556 | −4.04036 | 0 | 0.061844 | 0.256012 | 0 | 0 | −0.08036 | 0 | 0.806005 |
| 2557 | −4.01824 | 0 | 0.061483 | 0.243096 | 0 | 0 | −0.08643 | 0 | 0.797503 |
| 2558 | −3.56988 | 0 | 0.053091 | 0.266641 | 0 | 0 | −0.08357 | 0 | 0.691324 |
| 2559 | −3.54605 | 0 | 0.05263 | 0.252344 | 0 | 0 | −0.08876 | 0 | 0.67417 |
| 2560 | −3.79644 | 0 | 0.057253 | 0.238218 | 0 | 0 | −0.08548 | 0 | 0.719035 |
| 2561 | −3.77495 | 0 | 0.056938 | 0.226016 | 0 | 0 | −0.09036 | 0 | 0.713252 |
| 2562 | −3.84172 | 0 | 0.057764 | 0.243943 | 0 | 0 | −0.09219 | 0 | 0.707459 |
| 2563 | −3.62541 | 0 | 0.054633 | 0.239955 | 0 | 0 | 0 | −0.15181 | 0.668939 |
| 2564 | −3.77181 | 0 | 0.057991 | 0.279272 | 0 | 0 | 0 | −0.16478 | 0.799223 |
| 2565 | −4.06868 | 0 | 0.063523 | 0.243788 | 0 | 0 | 0 | −0.18507 | 0.835968 |
| 2566 | −4.0998 | 0 | 0.06392 | 0.265104 | 0 | 0 | 0 | −0.19147 | 0.846738 |
| 2567 | −4.078 | 0 | 0.063893 | 0.257505 | 0 | 0 | 0 | −0.22229 | 0.865087 |
| 2568 | −3.59759 | 0 | 0.054325 | 0.264951 | 0 | 0 | 0 | −0.15914 | 0.700723 |
| 2569 | −3.54859 | 0 | 0.053784 | 0.256041 | 0 | 0 | 0 | −0.19135 | 0.711293 |
| 2570 | −3.82433 | 0 | 0.058695 | 0.240016 | 0 | 0 | 0 | −0.17748 | 0.745868 |
| 2571 | −3.78975 | 0 | 0.058533 | 0.23223 | 0 | 0 | 0 | −0.20886 | 0.769915 |
| 2572 | −3.8493 | 0 | 0.059199 | 0.250317 | 0 | 0 | 0 | −0.20269 | 0.76275 |
| 2573 | −3.65796 | 0 | 0.055813 | 0.25083 | 0 | 0 | 0 | 0 | 0.805023 |
| 2574 | −3.65132 | 0 | 0.055626 | 0.274442 | 0 | 0 | 0 | 0 | 0.813896 |
| 2575 | −3.61162 | 0 | 0.054961 | 0.257927 | 0 | 0 | 0 | 0 | 0.800631 |
| 2576 | −3.96236 | 0 | 0.0611 | 0.230622 | 0 | 0 | 0 | 0 | 0.826097 |
| 2577 | −3.94019 | 0 | 0.06078 | 0.216585 | 0 | 0 | 0 | 0 | 0.817049 |
| 2578 | −3.98338 | 0 | 0.061236 | 0.234347 | 0 | 0 | 0 | 0 | 0.816728 |
| 2579 | −3.49217 | 0 | 0.052365 | 0.240115 | 0 | 0 | 0 | 0 | 0.718801 |
| 2580 | −3.46297 | 0 | 0.051894 | 0.225174 | 0 | 0 | 0 | 0 | 0.706823 |
| 2581 | −3.47894 | 0 | 0.052014 | 0.245624 | 0 | 0 | 0 | 0 | 0.711469 |
| 2582 | −3.73987 | 0 | 0.056744 | 0.216018 | 0 | 0 | 0 | 0 | 0.738516 |
| 2583 | −3.46289 | 0 | 0.050763 | 0 | 0.106952 | −0.08478 | −0.06575 | 0 | 0.597715 |
| 2584 | −3.8387 | 0 | 0.058426 | 0 | 0.106261 | −0.08616 | −0.06408 | 0 | 0.764997 |
| 2585 | −3.63317 | 0 | 0.054343 | 0 | 0.091069 | −0.08951 | −0.06782 | 0 | 0.666023 |
| 2586 | −3.65459 | 0 | 0.0547 | 0 | 0.097573 | −0.12037 | −0.06739 | 0 | 0.678616 |
| 2587 | −3.65596 | 0 | 0.054586 | 0 | 0.084183 | −0.09187 | −0.07301 | 0 | 0.662016 |
| 2588 | −3.50032 | 0 | 0.051888 | 0 | 0.12895 | −0.05753 | 0 | −0.14304 | 0.588835 |
| 2589 | −3.87481 | 0 | 0.059829 | 0 | 0.135809 | −0.05594 | 0 | −0.16907 | 0.784428 |
| 2590 | −3.66166 | 0 | 0.055499 | 0 | 0.113941 | −0.06073 | 0 | −0.15742 | 0.671722 |
| 2591 | −3.66965 | 0 | 0.055701 | 0 | 0.126958 | −0.09204 | 0 | −0.16269 | 0.69182 |
| 2592 | −3.64879 | 0 | 0.055574 | 0 | 0.117291 | −0.05811 | 0 | −0.19147 | 0.704404 |
| 2593 | −3.37472 | 0 | 0.050107 | 0 | 0.085515 | −0.09301 | 0 | 0 | 0.655645 |
| 2594 | −3.45802 | 0 | 0.052291 | 0 | 0.106739 | −0.1001 | 0 | 0 | 0.763064 |
| 2595 | −3.77962 | 0 | 0.058085 | 0 | 0.082666 | −0.0979 | 0 | 0 | 0.796063 |
| 2596 | −3.79201 | 0 | 0.05821 | 0 | 0.090896 | −0.13045 | 0 | 0 | 0.802661 |
| 2597 | −3.78439 | 0 | 0.057995 | 0 | 0.075224 | −0.09929 | 0 | 0 | 0.788432 |
| 2598 | −3.33123 | 0 | 0.049507 | 0 | 0.09193 | −0.12803 | 0 | 0 | 0.677047 |
| 2599 | −3.33091 | 0 | 0.049338 | 0 | 0.075695 | −0.09805 | 0 | 0 | 0.654943 |
| 2600 | −3.57208 | 0 | 0.054048 | 0 | 0.076028 | −0.12718 | 0 | 0 | 0.723099 |
| 2601 | −3.56742 | 0 | 0.053931 | 0 | 0.058911 | −0.10214 | 0 | 0 | 0.712566 |
| 2602 | −3.60693 | 0 | 0.054366 | 0 | 0.072886 | −0.12197 | 0 | 0 | 0.706719 |
| 2603 | −3.51848 | 0 | 0.051872 | 0 | 0.127664 | 0 | −0.06259 | −0.14832 | 0.580152 |
| 2604 | −3.88805 | 0 | 0.05976 | 0 | 0.13398 | 0 | −0.0589 | −0.17306 | 0.777612 |
| 2605 | −3.67122 | 0 | 0.055341 | 0 | 0.112785 | 0 | −0.06274 | −0.16204 | 0.663597 |
| 2606 | −3.69761 | 0 | 0.055683 | 0 | 0.118701 | 0 | −0.06306 | −0.17041 | 0.67124 |
| 2607 | −3.66221 | 0 | 0.055476 | 0 | 0.116449 | 0 | −0.06848 | −0.1963 | 0.697726 |
| 2608 | −3.3853 | 0 | 0.049859 | 0 | 0.074542 | 0 | −0.06121 | 0 | 0.64311 |
| 2609 | −3.47087 | 0 | 0.052105 | 0 | 0.091358 | 0 | −0.05027 | 0 | 0.751894 |
| 2610 | −3.78308 | 0 | 0.057731 | 0 | 0.070513 | 0 | −0.05947 | 0 | 0.784918 |
| 2611 | −3.80801 | 0 | 0.057878 | 0 | 0.069361 | 0 | −0.05852 | 0 | 0.777765 |
| 2612 | −3.79326 | 0 | 0.057691 | 0 | 0.061975 | 0 | −0.0647 | 0 | 0.775572 |
| 2613 | −3.36761 | 0 | 0.049477 | 0 | 0.071636 | 0 | −0.05935 | 0 | 0.648346 |
| 2614 | −3.34779 | 0 | 0.049159 | 0 | 0.063416 | 0 | −0.06449 | 0 | 0.640923 |
| 2615 | −3.59164 | 0 | 0.053751 | 0 | 0.057585 | 0 | −0.06285 | 0 | 0.69636 |
| 2616 | −3.57466 | 0 | 0.053573 | 0 | 0.046914 | 0 | −0.06768 | 0 | 0.698088 |
| 2617 | −3.6304 | 0 | 0.054126 | 0 | 0.055462 | 0 | −0.06827 | 0 | 0.680048 |
| 2618 | −3.43024 | 0 | 0.051227 | 0 | 0.104068 | 0 | 0 | −0.15306 | 0.64056 |
| 2619 | −3.51931 | 0 | 0.053627 | 0 | 0.12697 | 0 | 0 | −0.16351 | 0.761886 |
| 2620 | −3.8345 | 0 | 0.059527 | 0 | 0.106842 | 0 | 0 | −0.18187 | 0.81089 |
| 2621 | −3.84937 | 0 | 0.059572 | 0 | 0.114933 | 0 | 0 | −0.18879 | 0.811196 |
| 2622 | −3.82652 | 0 | 0.059571 | 0 | 0.111569 | 0 | 0 | −0.21701 | 0.834037 |
| 2623 | −3.39532 | 0 | 0.050654 | 0 | 0.107962 | 0 | 0 | −0.16075 | 0.655326 |
| 2624 | −3.34376 | 0 | 0.05013 | 0 | 0.103872 | 0 | 0 | −0.18931 | 0.673569 |
| 2625 | −3.61684 | 0 | 0.055074 | 0 | 0.094995 | 0 | 0 | −0.17619 | 0.717661 |
| 2626 | −3.5823 | 0 | 0.054952 | 0 | 0.08721 | 0 | 0 | −0.20467 | 0.746676 |
| 2627 | −3.63003 | 0 | 0.055334 | 0 | 0.101034 | 0 | 0 | −0.20024 | 0.72814 |
| 2628 | −3.44142 | 0 | 0.052068 | 0 | 0.070171 | 0 | 0 | 0 | 0.775848 |
| 2629 | −3.42327 | 0 | 0.051606 | 0 | 0.066153 | 0 | 0 | 0 | 0.772983 |
| 2630 | −3.39034 | 0 | 0.051111 | 0 | 0.056691 | 0 | 0 | 0 | 0.767673 |
| 2631 | −3.75569 | 0 | 0.057545 | 0 | 0.048062 | 0 | 0 | 0 | 0.803713 |
| 2632 | −3.74008 | 0 | 0.057384 | 0 | 0.03515 | 0 | 0 | 0 | 0.801569 |
| 2633 | −3.77128 | 0 | 0.057576 | 0 | 0.044986 | 0 | 0 | 0 | 0.7916 |
| 2634 | −3.30835 | 0 | 0.049074 | 0 | 0.051803 | 0 | 0 | 0 | 0.686074 |
| 2635 | −3.28147 | 0 | 0.048716 | 0 | 0.039389 | 0 | 0 | 0 | 0.683848 |
| 2636 | −3.29352 | 0 | 0.048658 | 0 | 0.046044 | 0 | 0 | 0 | 0.673742 |
| 2637 | −3.55327 | 0 | 0.053527 | 0 | 0.030948 | 0 | 0 | 0 | 0.720659 |
| 2638 | −3.5194 | 0 | 0.051878 | 0 | 0 | −0.03591 | −0.04998 | −0.12754 | 0.584604 |
| 2639 | −3.89434 | 0 | 0.0598 | 0 | 0 | −0.03311 | −0.04652 | −0.15273 | 0.777771 |
| 2640 | −3.66803 | 0 | 0.055336 | 0 | 0 | −0.04133 | −0.05109 | −0.14341 | 0.673261 |
| 2641 | −3.68314 | 0 | 0.055594 | 0 | 0 | −0.06999 | −0.05162 | −0.14589 | 0.686848 |
| 2642 | −3.65836 | 0 | 0.05544 | 0 | 0 | −0.03804 | −0.05732 | −0.17568 | 0.705143 |
| 2643 | −3.38522 | 0 | 0.050057 | 0 | 0 | −0.07437 | −0.0519 | 0 | 0.653427 |
| 2644 | −3.48329 | 0 | 0.052461 | 0 | 0 | −0.07661 | −0.03915 | 0 | 0.756632 |
| 2645 | −3.78942 | 0 | 0.05802 | 0 | 0 | −0.07928 | −0.0502 | 0 | 0.793613 |
| 2646 | −3.80493 | 0 | 0.058182 | 0 | 0 | −0.10967 | −0.04984 | 0 | 0.797525 |
| 2647 | −3.79564 | 0 | 0.05796 | 0 | 0 | −0.08153 | −0.057 | 0 | 0.787054 |
| 2648 | −3.34722 | 0 | 0.049531 | 0 | 0 | −0.10726 | −0.0502 | 0 | 0.671903 |
| 2649 | −3.3391 | 0 | 0.049274 | 0 | 0 | −0.08098 | −0.05667 | 0 | 0.655043 |
| 2650 | −3.57745 | 0 | 0.053922 | 0 | 0 | −0.10976 | −0.0547 | 0 | 0.721262 |
| 2651 | −3.56744 | 0 | 0.053749 | 0 | 0 | −0.08824 | −0.0611 | 0 | 0.714948 |
| 2652 | −3.61461 | 0 | 0.054277 | 0 | 0 | −0.10503 | −0.06112 | 0 | 0.706027 |
| 2653 | −3.42646 | 0 | 0.051195 | 0 | 0 | −0.04607 | 0 | −0.13405 | 0.649708 |
| 2654 | −3.53217 | 0 | 0.053778 | 0 | 0 | −0.04587 | 0 | −0.14203 | 0.762569 |
| 2655 | −3.83794 | 0 | 0.059562 | 0 | 0 | −0.04627 | 0 | −0.16355 | 0.815809 |
| 2656 | −3.84422 | 0 | 0.059571 | 0 | 0 | −0.07722 | 0 | −0.16426 | 0.821188 |
| 2657 | −3.82949 | 0 | 0.059586 | 0 | 0 | −0.04147 | 0 | −0.19656 | 0.837367 |
| 2658 | −3.3763 | 0 | 0.050494 | 0 | 0 | −0.07806 | 0 | −0.13559 | 0.671822 |
| 2659 | −3.33881 | 0 | 0.05008 | 0 | 0 | −0.04522 | 0 | −0.16896 | 0.681634 |
| 2660 | −3.60165 | 0 | 0.054996 | 0 | 0 | −0.07912 | 0 | −0.15419 | 0.735724 |
| 2661 | −3.57502 | 0 | 0.054908 | 0 | 0 | −0.05059 | 0 | −0.18698 | 0.758116 |
| 2662 | −3.61616 | 0 | 0.055238 | 0 | 0 | −0.07022 | 0 | −0.17709 | 0.74297 |
| 2663 | −3.45149 | 0 | 0.052405 | 0 | 0 | −0.08877 | 0 | 0 | 0.783636 |
| 2664 | −3.4181 | 0 | 0.051874 | 0 | 0 | −0.12402 | 0 | 0 | 0.791558 |
| 2665 | −3.39297 | 0 | 0.05138 | 0 | 0 | −0.09524 | 0 | 0 | 0.778052 |
| 2666 | −3.75235 | 0 | 0.057851 | 0 | 0 | −0.12038 | 0 | 0 | 0.824138 |
| 2667 | −3.74009 | 0 | 0.057648 | 0 | 0 | −0.09806 | 0 | 0 | 0.815147 |
| 2668 | −3.76674 | 0 | 0.057872 | 0 | 0 | −0.11506 | 0 | 0 | 0.812921 |
| 2669 | −3.28891 | 0 | 0.04915 | 0 | 0 | −0.11611 | 0 | 0 | 0.710239 |
| 2670 | −3.27172 | 0 | 0.048838 | 0 | 0 | −0.0935 | 0 | 0 | 0.699351 |
| 2671 | −3.27025 | 0 | 0.048681 | 0 | 0 | −0.11383 | 0 | 0 | 0.698571 |
| 2672 | −3.53791 | 0 | 0.053694 | 0 | 0 | −0.1163 | 0 | 0 | 0.746077 |
| 2673 | −3.43654 | 0 | 0.051122 | 0 | 0 | 0 | −0.04657 | −0.138 | 0.642548 |
| 2674 | −3.54378 | 0 | 0.053774 | 0 | 0 | 0 | −0.03448 | −0.14667 | 0.757184 |
| 2675 | −3.84373 | 0 | 0.059444 | 0 | 0 | 0 | −0.04179 | −0.16682 | 0.809945 |
| 2676 | −3.85985 | 0 | 0.059496 | 0 | 0 | 0 | −0.0429 | −0.17111 | 0.80746 |
| 2677 | −3.83942 | 0 | 0.059526 | 0 | 0 | 0 | −0.04865 | −0.19954 | 0.832156 |
| 2678 | −3.40478 | 0 | 0.050592 | 0 | 0 | 0 | −0.04629 | −0.14395 | 0.654792 |
| 2679 | −3.35159 | 0 | 0.050049 | 0 | 0 | 0 | −0.05103 | −0.17321 | 0.675765 |
| 2680 | −3.6186 | 0 | 0.054905 | 0 | 0 | 0 | −0.04836 | −0.16127 | 0.719602 |
| 2681 | −3.58119 | 0 | 0.054752 | 0 | 0 | 0 | −0.05304 | −0.19091 | 0.751783 |
| 2682 | −3.63523 | 0 | 0.055196 | 0 | 0 | 0 | −0.05435 | −0.18323 | 0.72937 |
| 2683 | −3.45653 | 0 | 0.052161 | 0 | 0 | 0 | −0.03795 | 0 | 0.773286 |
| 2684 | −3.43765 | 0 | 0.051709 | 0 | 0 | 0 | −0.03649 | 0 | 0.770618 |
| 2685 | −3.40414 | 0 | 0.051197 | 0 | 0 | 0 | −0.04301 | 0 | 0.766386 |
| 2686 | −3.76121 | 0 | 0.057485 | 0 | 0 | 0 | −0.04878 | 0 | 0.801857 |
| 2687 | −3.74393 | 0 | 0.057293 | 0 | 0 | 0 | −0.05503 | 0 | 0.801072 |
| 2688 | −3.78073 | 0 | 0.057572 | 0 | 0 | 0 | −0.05487 | 0 | 0.790168 |
| 2689 | −3.31538 | 0 | 0.049046 | 0 | 0 | 0 | −0.04964 | 0 | 0.685073 |
| 2690 | −3.28438 | 0 | 0.048624 | 0 | 0 | 0 | −0.05514 | 0 | 0.684625 |
| 2691 | −3.30183 | 0 | 0.04865 | 0 | 0 | 0 | −0.05443 | 0 | 0.673565 |
| 2692 | −3.55569 | 0 | 0.053417 | 0 | 0 | 0 | −0.05976 | 0 | 0.720788 |
| 2693 | −3.51642 | 0 | 0.053799 | 0 | 0 | 0 | 0 | −0.1557 | 0.785691 |
| 2694 | −3.48734 | 0 | 0.053233 | 0 | 0 | 0 | 0 | −0.16155 | 0.787739 |
| 2695 | −3.44338 | 0 | 0.052814 | 0 | 0 | 0 | 0 | −0.19402 | 0.804286 |
| 2696 | −3.8114 | 0 | 0.059261 | 0 | 0 | 0 | 0 | −0.18166 | 0.836743 |
| 2697 | −3.79211 | 0 | 0.059379 | 0 | 0 | 0 | 0 | −0.21384 | 0.863042 |
| 2698 | −3.81741 | 0 | 0.059369 | 0 | 0 | 0 | 0 | −0.20303 | 0.846213 |
| 2699 | −3.34716 | 0 | 0.050234 | 0 | 0 | 0 | 0 | −0.15143 | 0.694988 |
| 2700 | −3.29088 | 0 | 0.049725 | 0 | 0 | 0 | 0 | −0.1833 | 0.720568 |
| 2701 | −3.30043 | 0 | 0.049569 | 0 | 0 | 0 | 0 | −0.17706 | 0.706955 |
| 2702 | −3.56268 | 0 | 0.054709 | 0 | 0 | 0 | 0 | −0.19517 | 0.771718 |
| 2703 | −3.414 | 0 | 0.051676 | 0 | 0 | 0 | 0 | 0 | 0.790026 |
| 2704 | −3.37804 | 0 | 0.051176 | 0 | 0 | 0 | 0 | 0 | 0.787211 |
| 2705 | −3.37893 | 0 | 0.05098 | 0 | 0 | 0 | 0 | 0 | 0.781522 |
| 2706 | −3.73112 | 0 | 0.057272 | 0 | 0 | 0 | 0 | 0 | 0.813471 |
| 2707 | −3.24999 | 0 | 0.04833 | 0 | 0 | 0 | 0 | 0 | 0.706155 |
| 2708 | −0.23087 | 0 | 0 | 0.153233 | 0.082475 | −0.05675 | −0.07731 | 0 | 0.85009 |
| 2709 | −0.25131 | 0 | 0 | 0.114305 | 0.078872 | −0.04908 | −0.07849 | 0 | 0.827082 |
| 2710 | −0.22821 | 0 | 0 | 0.118366 | 0.063222 | −0.05932 | −0.0842 | 0 | 0.835643 |
| 2711 | −0.23722 | 0 | 0 | 0.130357 | 0.067427 | −0.09644 | −0.08222 | 0 | 0.840144 |
| 2712 | −0.24729 | 0 | 0 | 0.118082 | 0.055403 | −0.06226 | −0.08946 | 0 | 0.823539 |
| 2713 | −0.19909 | 0 | 0 | 0.145673 | 0.100874 | −0.03752 | 0 | −0.11646 | 0.843898 |
| 2714 | −0.20767 | 0 | 0 | 0.111018 | 0.104532 | −0.0295 | 0 | −0.13871 | 0.836857 |
| 2715 | −0.1884 | 0 | 0 | 0.113253 | 0.085032 | −0.03909 | 0 | −0.13386 | 0.838302 |
| 2716 | −0.19318 | 0 | 0 | 0.125402 | 0.093463 | −0.0765 | 0 | −0.14082 | 0.850778 |
| 2717 | −0.18126 | 0 | 0 | 0.116913 | 0.086607 | −0.03727 | 0 | −0.17318 | 0.86001 |
| 2718 | −0.19259 | 0 | 0 | 0.134954 | 0.058857 | −0.06781 | 0 | 0 | 0.874785 |
| 2719 | −0.19339 | 0 | 0 | 0.147302 | 0.083189 | −0.07034 | 0 | 0 | 0.899592 |
| 2720 | −0.20585 | 0 | 0 | 0.098538 | 0.055572 | −0.06483 | 0 | 0 | 0.861381 |
| 2721 | −0.21562 | 0 | 0 | 0.111518 | 0.059393 | −0.09944 | 0 | 0 | 0.865786 |
| 2722 | −0.22478 | 0 | 0 | 0.097772 | 0.048105 | −0.0658 | 0 | 0 | 0.850094 |
| 2723 | −0.18939 | 0 | 0 | 0.156023 | 0.063036 | −0.11415 | 0 | 0 | 0.897976 |
| 2724 | −0.19952 | 0 | 0 | 0.14 | 0.046135 | −0.0757 | 0 | 0 | 0.877615 |
| 2725 | −0.19417 | 0 | 0 | 0.115033 | 0.045224 | −0.10519 | 0 | 0 | 0.873075 |
| 2726 | −0.19814 | 0 | 0 | 0.101569 | 0.029526 | −0.07626 | 0 | 0 | 0.862457 |
| 2727 | −0.21582 | 0 | 0 | 0.112556 | 0.042655 | −0.09918 | 0 | 0 | 0.85503 |
| 2728 | −0.21739 | 0 | 0 | 0.16016 | 0.101882 | 0 | −0.07635 | −0.11915 | 0.837384 |
| 2729 | −0.22363 | 0 | 0 | 0.125915 | 0.106922 | 0 | −0.07665 | −0.1397 | 0.831323 |
| 2730 | −0.20624 | 0 | 0 | 0.128439 | 0.086279 | 0 | −0.08249 | −0.1359 | 0.832781 |
| 2731 | −0.22041 | 0 | 0 | 0.143176 | 0.086051 | 0 | −0.08076 | −0.14588 | 0.833364 |
| 2732 | −0.199 | 0 | 0 | 0.134093 | 0.088162 | 0 | −0.08828 | −0.17562 | 0.856633 |
| 2733 | −0.2164 | 0 | 0 | 0.149955 | 0.051763 | 0 | −0.07604 | 0 | 0.864888 |
| 2734 | −0.21599 | 0 | 0 | 0.161523 | 0.073429 | 0 | −0.06132 | 0 | 0.888843 |
| 2735 | −0.22789 | 0 | 0 | 0.115275 | 0.049711 | 0 | −0.07886 | 0 | 0.852581 |
| 2736 | −0.24712 | 0 | 0 | 0.130255 | 0.04437 | 0 | −0.07696 | 0 | 0.845147 |
| 2737 | −0.248 | 0 | 0 | 0.116102 | 0.041628 | 0 | −0.08234 | 0 | 0.840683 |
| 2738 | −0.22607 | 0 | 0 | 0.172342 | 0.0446 | 0 | −0.07243 | 0 | 0.872259 |
| 2739 | −0.22542 | 0 | 0 | 0.157312 | 0.037822 | 0 | −0.08031 | 0 | 0.866826 |
| 2740 | −0.22863 | 0 | 0 | 0.133279 | 0.030078 | 0 | −0.0823 | 0 | 0.850796 |
| 2741 | −0.22404 | 0 | 0 | 0.119917 | 0.021563 | 0 | −0.08799 | 0 | 0.851914 |
| 2742 | −0.24991 | 0 | 0 | 0.132103 | 0.029285 | 0 | −0.08617 | 0 | 0.833471 |
| 2743 | −0.17839 | 0 | 0 | 0.143086 | 0.076517 | 0 | 0 | −0.12603 | 0.863836 |
| 2744 | −0.17419 | 0 | 0 | 0.158853 | 0.102394 | 0 | 0 | −0.1326 | 0.896521 |
| 2745 | −0.17764 | 0 | 0 | 0.112084 | 0.080164 | 0 | 0 | −0.14858 | 0.867691 |
| 2746 | −0.19202 | 0 | 0 | 0.127938 | 0.080587 | 0 | 0 | −0.15659 | 0.868751 |
| 2747 | −0.17507 | 0 | 0 | 0.116701 | 0.082937 | 0 | 0 | −0.18344 | 0.884901 |
| 2748 | −0.18219 | 0 | 0 | 0.166452 | 0.074368 | 0 | 0 | −0.13772 | 0.881123 |
| 2749 | −0.15983 | 0 | 0 | 0.155458 | 0.074414 | 0 | 0 | −0.17428 | 0.899376 |
| 2750 | −0.17634 | 0 | 0 | 0.129089 | 0.062806 | 0 | 0 | −0.15274 | 0.868509 |
| 2751 | −0.15086 | 0 | 0 | 0.118788 | 0.059018 | 0 | 0 | −0.18347 | 0.893025 |
| 2752 | −0.179 | 0 | 0 | 0.130608 | 0.068073 | 0 | 0 | −0.17951 | 0.875739 |
| 2753 | −0.18832 | 0 | 0 | 0.144339 | 0.04936 | 0 | 0 | 0 | 0.901711 |
| 2754 | −0.19772 | 0 | 0 | 0.168011 | 0.039295 | 0 | 0 | 0 | 0.908502 |
| 2755 | −0.1978 | 0 | 0 | 0.15027 | 0.034348 | 0 | 0 | 0 | 0.902131 |
| 2756 | −0.21064 | 0 | 0 | 0.114449 | 0.021377 | 0 | 0 | 0 | 0.870072 |
| 2757 | −0.20707 | 0 | 0 | 0.099904 | 0.013085 | 0 | 0 | 0 | 0.869098 |
| 2758 | −0.22982 | 0 | 0 | 0.113238 | 0.02023 | 0 | 0 | 0 | 0.856511 |
| 2759 | −0.19623 | 0 | 0 | 0.153618 | 0.022255 | 0 | 0 | 0 | 0.888544 |
| 2760 | −0.19188 | 0 | 0 | 0.137713 | 0.010967 | 0 | 0 | 0 | 0.886179 |
| 2761 | −0.2064 | 0 | 0 | 0.157058 | 0.016086 | 0 | 0 | 0 | 0.885482 |
| 2762 | −0.20862 | 0 | 0 | 0.11563 | 0.002714 | 0 | 0 | 0 | 0.864293 |
| 2763 | −0.21837 | 0 | 0 | 0.16839 | 0 | −0.01672 | −0.06777 | −0.10328 | 0.841325 |
| 2764 | −0.22705 | 0 | 0 | 0.135706 | 0 | −0.00738 | −0.06782 | −0.12475 | 0.833092 |
| 2765 | −0.20439 | 0 | 0 | 0.13511 | 0 | −0.02112 | −0.07495 | −0.12213 | 0.840581 |
| 2766 | −0.21066 | 0 | 0 | 0.149217 | 0 | −0.05674 | −0.07313 | −0.12704 | 0.849914 |
| 2767 | −0.19775 | 0 | 0 | 0.141503 | 0 | −0.01881 | −0.08116 | −0.16066 | 0.863807 |
| 2768 | −0.20688 | 0 | 0 | 0.153327 | 0 | −0.05221 | −0.07009 | 0 | 0.873142 |
| 2769 | −0.20932 | 0 | 0 | 0.166699 | 0 | −0.04811 | −0.05275 | 0 | 0.892563 |
| 2770 | −0.21931 | 0 | 0 | 0.118301 | 0 | −0.04949 | −0.07292 | 0 | 0.860411 |
| 2771 | −0.22928 | 0 | 0 | 0.1323 | 0 | −0.08329 | −0.07103 | 0 | 0.86356 |
| 2772 | −0.23846 | 0 | 0 | 0.118535 | 0 | −0.05188 | −0.07746 | 0 | 0.849788 |
| 2773 | −0.20393 | 0 | 0 | 0.174954 | 0 | −0.09736 | −0.06569 | 0 | 0.89426 |
| 2774 | −0.21304 | 0 | 0 | 0.159335 | 0 | −0.06304 | −0.0755 | 0 | 0.877866 |
| 2775 | −0.20683 | 0 | 0 | 0.13368 | 0 | −0.09266 | −0.07759 | 0 | 0.872979 |
| 2776 | −0.21003 | 0 | 0 | 0.119785 | 0 | −0.06736 | −0.08478 | 0 | 0.864908 |
| 2777 | −0.22927 | 0 | 0 | 0.132843 | 0 | −0.08744 | −0.08191 | 0 | 0.855647 |
| 2778 | −0.17642 | 0 | 0 | 0.149323 | 0 | −0.03042 | 0 | −0.11211 | 0.871236 |
| 2779 | −0.1772 | 0 | 0 | 0.168045 | 0 | −0.02563 | 0 | −0.11623 | 0.897398 |
| 2780 | −0.17719 | 0 | 0 | 0.118906 | 0 | −0.02585 | 0 | −0.13566 | 0.87376 |
| 2781 | −0.1848 | 0 | 0 | 0.134242 | 0 | −0.05908 | 0 | −0.13876 | 0.881888 |
| 2782 | −0.17557 | 0 | 0 | 0.12436 | 0 | −0.02148 | 0 | −0.16927 | 0.889979 |
| 2783 | −0.17058 | 0 | 0 | 0.172055 | 0 | −0.07407 | 0 | −0.11757 | 0.898444 |
| 2784 | −0.15735 | 0 | 0 | 0.161661 | 0 | −0.03145 | 0 | −0.15945 | 0.906607 |
| 2785 | −0.16432 | 0 | 0 | 0.132677 | 0 | −0.06826 | 0 | −0.1362 | 0.886315 |
| 2786 | −0.14595 | 0 | 0 | 0.122753 | 0 | −0.03622 | 0 | −0.17108 | 0.90293 |
| 2787 | −0.16931 | 0 | 0 | 0.135429 | 0 | −0.05911 | 0 | −0.16276 | 0.891714 |
| 2788 | −0.1791 | 0 | 0 | 0.148068 | 0 | −0.06431 | 0 | 0 | 0.908939 |
| 2789 | −0.17691 | 0 | 0 | 0.170625 | 0 | −0.10799 | 0 | 0 | 0.92742 |
| 2790 | −0.18621 | 0 | 0 | 0.152117 | 0 | −0.0734 | 0 | 0 | 0.911255 |
| 2791 | −0.19055 | 0 | 0 | 0.114036 | 0 | −0.09744 | 0 | 0 | 0.889788 |
| 2792 | −0.19383 | 0 | 0 | 0.098815 | 0 | −0.0732 | 0 | 0 | 0.88062 |
| 2793 | −0.21125 | 0 | 0 | 0.112999 | 0 | −0.09091 | 0 | 0 | 0.87525 |
| 2794 | −0.17289 | 0 | 0 | 0.15457 | 0 | −0.10684 | 0 | 0 | 0.910549 |
| 2795 | −0.17701 | 0 | 0 | 0.137206 | 0 | −0.07877 | 0 | 0 | 0.898159 |
| 2796 | −0.18331 | 0 | 0 | 0.157581 | 0 | −0.10622 | 0 | 0 | 0.907643 |
| 2797 | −0.18611 | 0 | 0 | 0.113351 | 0 | −0.10119 | 0 | 0 | 0.885779 |
| 2798 | −0.19205 | 0 | 0 | 0.161905 | 0 | 0 | −0.06742 | −0.1137 | 0.865547 |
| 2799 | −0.19031 | 0 | 0 | 0.178059 | 0 | 0 | −0.04976 | −0.11755 | 0.891088 |
| 2800 | −0.1912 | 0 | 0 | 0.132229 | 0 | 0 | −0.06837 | −0.13577 | 0.868897 |
| 2801 | −0.20529 | 0 | 0 | 0.148999 | 0 | 0 | −0.06737 | −0.14254 | 0.869102 |
| 2802 | −0.18969 | 0 | 0 | 0.138824 | 0 | 0 | −0.07276 | −0.16928 | 0.885948 |
| 2803 | −0.1953 | 0 | 0 | 0.185564 | 0 | 0 | −0.06444 | −0.12481 | 0.881399 |
| 2804 | −0.1735 | 0 | 0 | 0.176101 | 0 | 0 | −0.07239 | −0.16172 | 0.902184 |
| 2805 | −0.18783 | 0 | 0 | 0.148207 | 0 | 0 | −0.07479 | −0.14133 | 0.872194 |
| 2806 | −0.16219 | 0 | 0 | 0.138548 | 0 | 0 | −0.08105 | −0.17305 | 0.899324 |
| 2807 | −0.19162 | 0 | 0 | 0.152086 | 0 | 0 | −0.07871 | −0.16706 | 0.880035 |
| 2808 | −0.19956 | 0 | 0 | 0.160067 | 0 | 0 | −0.05614 | 0 | 0.898658 |
| 2809 | −0.20764 | 0 | 0 | 0.181937 | 0 | 0 | −0.05197 | 0 | 0.90529 |
| 2810 | −0.20891 | 0 | 0 | 0.165306 | 0 | 0 | −0.05944 | 0 | 0.899486 |
| 2811 | −0.22086 | 0 | 0 | 0.130315 | 0 | 0 | −0.0751 | 0 | 0.86971 |
| 2812 | −0.21802 | 0 | 0 | 0.115878 | 0 | 0 | −0.08012 | 0 | 0.869111 |
| 2813 | −0.24119 | 0 | 0 | 0.129989 | 0 | 0 | −0.07731 | 0 | 0.855539 |
| 2814 | −0.20704 | 0 | 0 | 0.168137 | 0 | 0 | −0.06923 | 0 | 0.8868 |
| 2815 | −0.20326 | 0 | 0 | 0.152607 | 0 | 0 | −0.07689 | 0 | 0.885546 |
| 2816 | −0.21797 | 0 | 0 | 0.172547 | 0 | 0 | −0.07333 | 0 | 0.883826 |
| 2817 | −0.22006 | 0 | 0 | 0.130646 | 0 | 0 | −0.08442 | 0 | 0.863534 |
| 2818 | −0.1599 | 0 | 0 | 0.161015 | 0 | 0 | 0 | −0.12778 | 0.90833 |
| 2819 | −0.16264 | 0 | 0 | 0.185942 | 0 | 0 | 0 | −0.13777 | 0.922911 |
| 2820 | −0.14746 | 0 | 0 | 0.173224 | 0 | 0 | 0 | −0.17184 | 0.934758 |
| 2821 | −0.16598 | 0 | 0 | 0.132508 | 0 | 0 | 0 | −0.15351 | 0.896702 |
| 2822 | −0.1446 | 0 | 0 | 0.121249 | 0 | 0 | 0 | −0.183 | 0.91769 |
| 2823 | −0.17115 | 0 | 0 | 0.135951 | 0 | 0 | 0 | −0.17534 | 0.903129 |
| 2824 | −0.16338 | 0 | 0 | 0.166333 | 0 | 0 | 0 | −0.13354 | 0.899128 |
| 2825 | −0.13803 | 0 | 0 | 0.15487 | 0 | 0 | 0 | −0.17103 | 0.920983 |
| 2826 | −0.15486 | 0 | 0 | 0.175409 | 0 | 0 | 0 | −0.16735 | 0.920488 |
| 2827 | −0.14761 | 0 | 0 | 0.134112 | 0 | 0 | 0 | −0.17873 | 0.9107 |
| 2828 | −0.18448 | 0 | 0 | 0.163452 | 0 | 0 | 0 | 0 | 0.914979 |
| 2829 | −0.18222 | 0 | 0 | 0.145315 | 0 | 0 | 0 | 0 | 0.911154 |
| 2830 | −0.19401 | 0 | 0 | 0.166131 | 0 | 0 | 0 | 0 | 0.913072 |
| 2831 | −0.20497 | 0 | 0 | 0.110181 | 0 | 0 | 0 | 0 | 0.87743 |
| 2832 | −0.18898 | 0 | 0 | 0.150145 | 0 | 0 | 0 | 0 | 0.897085 |
| 2833 | −0.22702 | 0 | 0 | 0 | 0.125864 | −0.04625 | −0.05931 | −0.11224 | 0.812112 |
| 2834 | −0.22589 | 0 | 0 | 0 | 0.127378 | −0.03795 | −0.06226 | −0.13296 | 0.820274 |
| 2835 | −0.20814 | 0 | 0 | 0 | 0.10798 | −0.0471 | −0.06877 | −0.12875 | 0.822146 |
| 2836 | −0.21577 | 0 | 0 | 0 | 0.117594 | −0.08434 | −0.06489 | −0.13551 | 0.827279 |
| 2837 | −0.20194 | 0 | 0 | 0 | 0.110819 | −0.04616 | −0.07337 | −0.1668 | 0.843364 |
| 2838 | −0.21617 | 0 | 0 | 0 | 0.083555 | −0.07442 | −0.06044 | 0 | 0.849442 |
| 2839 | −0.21445 | 0 | 0 | 0 | 0.108122 | −0.0784 | −0.04367 | 0 | 0.871638 |
| 2840 | −0.22102 | 0 | 0 | 0 | 0.078374 | −0.07112 | −0.06588 | 0 | 0.849265 |
| 2841 | −0.23258 | 0 | 0 | 0 | 0.083844 | −0.10606 | −0.06222 | 0 | 0.847635 |
| 2842 | −0.24031 | 0 | 0 | 0 | 0.071607 | −0.07229 | −0.06906 | 0 | 0.837652 |
| 2843 | −0.2169 | 0 | 0 | 0 | 0.089495 | −0.1199 | −0.05364 | 0 | 0.861503 |
| 2844 | −0.2242 | 0 | 0 | 0 | 0.073049 | −0.08279 | −0.06327 | 0 | 0.850175 |
| 2845 | −0.21332 | 0 | 0 | 0 | 0.069349 | −0.11128 | −0.06795 | 0 | 0.854416 |
| 2846 | −0.21492 | 0 | 0 | 0 | 0.053174 | −0.08269 | −0.07527 | 0 | 0.85074 |
| 2847 | −0.23519 | 0 | 0 | 0 | 0.068065 | −0.10538 | −0.07168 | 0 | 0.836733 |
| 2848 | −0.18515 | 0 | 0 | 0 | 0.102402 | −0.05471 | 0 | −0.11792 | 0.846594 |
| 2849 | −0.18178 | 0 | 0 | 0 | 0.131823 | −0.05829 | 0 | −0.1225 | 0.875411 |
| 2850 | −0.17793 | 0 | 0 | 0 | 0.103023 | −0.05082 | 0 | −0.14066 | 0.860983 |
| 2851 | −0.18733 | 0 | 0 | 0 | 0.113572 | −0.08631 | 0 | −0.14562 | 0.864671 |
| 2852 | −0.17664 | 0 | 0 | 0 | 0.10628 | −0.04822 | 0 | −0.17444 | 0.875757 |
| 2853 | −0.1837 | 0 | 0 | 0 | 0.112312 | −0.09966 | 0 | −0.12489 | 0.864827 |
| 2854 | −0.16938 | 0 | 0 | 0 | 0.102407 | −0.05672 | 0 | −0.16331 | 0.876668 |
| 2855 | −0.17068 | 0 | 0 | 0 | 0.094837 | −0.09092 | 0 | −0.14152 | 0.865894 |
| 2856 | −0.15136 | 0 | 0 | 0 | 0.083146 | −0.05752 | 0 | −0.17393 | 0.885273 |
| 2857 | −0.17565 | 0 | 0 | 0 | 0.099612 | −0.08308 | 0 | −0.16812 | 0.870124 |
| 2858 | −0.18535 | 0 | 0 | 0 | 0.087496 | −0.0883 | 0 | 0 | 0.890362 |
| 2859 | −0.18679 | 0 | 0 | 0 | 0.091818 | −0.13279 | 0 | 0 | 0.90021 |
| 2860 | −0.19401 | 0 | 0 | 0 | 0.075323 | −0.09534 | 0 | 0 | 0.889741 |
| 2861 | −0.19457 | 0 | 0 | 0 | 0.06189 | −0.11451 | 0 | 0 | 0.876741 |
| 2862 | −0.19651 | 0 | 0 | 0 | 0.045463 | −0.08678 | 0 | 0 | 0.871525 |
| 2863 | −0.21511 | 0 | 0 | 0 | 0.06006 | −0.10761 | 0 | 0 | 0.861738 |
| 2864 | −0.18554 | 0 | 0 | 0 | 0.068266 | −0.12414 | 0 | 0 | 0.883712 |
| 2865 | −0.18736 | 0 | 0 | 0 | 0.04811 | −0.09217 | 0 | 0 | 0.877621 |
| 2866 | −0.19726 | 0 | 0 | 0 | 0.062877 | −0.12186 | 0 | 0 | 0.878011 |
| 2867 | −0.19241 | 0 | 0 | 0 | 0.04201 | −0.11302 | 0 | 0 | 0.87141 |
| 2868 | −0.20429 | 0 | 0 | 0 | 0.099727 | 0 | −0.05819 | −0.12205 | 0.837273 |
| 2869 | −0.19864 | 0 | 0 | 0 | 0.125694 | 0 | −0.04164 | −0.12712 | 0.865976 |
| 2870 | −0.19541 | 0 | 0 | 0 | 0.101826 | 0 | −0.06205 | −0.14311 | 0.853009 |
| 2871 | −0.21294 | 0 | 0 | 0 | 0.103779 | 0 | −0.05826 | −0.15066 | 0.845989 |
| 2872 | −0.19423 | 0 | 0 | 0 | 0.105236 | 0 | −0.06518 | −0.17689 | 0.868153 |
| 2873 | −0.21349 | 0 | 0 | 0 | 0.0995 | 0 | −0.05176 | −0.13322 | 0.841643 |
| 2874 | −0.18902 | 0 | 0 | 0 | 0.099444 | 0 | −0.0607 | −0.168 | 0.86795 |
| 2875 | −0.19917 | 0 | 0 | 0 | 0.085641 | 0 | −0.0646 | −0.14747 | 0.846108 |
| 2876 | −0.17126 | 0 | 0 | 0 | 0.081377 | 0 | −0.0718 | −0.17753 | 0.877546 |
| 2877 | −0.20305 | 0 | 0 | 0 | 0.091944 | 0 | −0.06799 | −0.17319 | 0.852012 |
| 2878 | −0.20881 | 0 | 0 | 0 | 0.073277 | 0 | −0.04467 | 0 | 0.87727 |
| 2879 | −0.22098 | 0 | 0 | 0 | 0.065305 | 0 | −0.03781 | 0 | 0.874232 |
| 2880 | −0.21871 | 0 | 0 | 0 | 0.059181 | 0 | −0.04591 | 0 | 0.875472 |
| 2881 | −0.22778 | 0 | 0 | 0 | 0.045 | 0 | −0.06306 | 0 | 0.85334 |
| 2882 | −0.22238 | 0 | 0 | 0 | 0.035522 | 0 | −0.06892 | 0 | 0.857979 |
| 2883 | −0.24753 | 0 | 0 | 0 | 0.044657 | 0 | −0.06519 | 0 | 0.838763 |
| 2884 | −0.22239 | 0 | 0 | 0 | 0.04774 | 0 | −0.05408 | 0 | 0.856714 |
| 2885 | −0.21504 | 0 | 0 | 0 | 0.035936 | 0 | −0.06209 | 0 | 0.862873 |
| 2886 | −0.23393 | 0 | 0 | 0 | 0.043522 | 0 | −0.05679 | 0 | 0.851086 |
| 2887 | −0.22809 | 0 | 0 | 0 | 0.026838 | 0 | −0.07087 | 0 | 0.846972 |
| 2888 | −0.16934 | 0 | 0 | 0 | 0.103269 | 0 | 0 | −0.13383 | 0.88521 |
| 2889 | −0.17805 | 0 | 0 | 0 | 0.102058 | 0 | 0 | −0.14377 | 0.888719 |
| 2890 | −0.15892 | 0 | 0 | 0 | 0.102566 | 0 | 0 | −0.17665 | 0.907637 |
| 2891 | −0.1742 | 0 | 0 | 0 | 0.080759 | 0 | 0 | −0.15795 | 0.876702 |
| 2892 | −0.15051 | 0 | 0 | 0 | 0.075783 | 0 | 0 | −0.18628 | 0.901691 |
| 2893 | −0.17949 | 0 | 0 | 0 | 0.087091 | 0 | 0 | −0.18034 | 0.881439 |
| 2894 | −0.18074 | 0 | 0 | 0 | 0.078485 | 0 | 0 | −0.13847 | 0.866161 |
| 2895 | −0.15259 | 0 | 0 | 0 | 0.072645 | 0 | 0 | −0.17364 | 0.893831 |
| 2896 | −0.17464 | 0 | 0 | 0 | 0.081583 | 0 | 0 | −0.17095 | 0.882888 |
| 2897 | −0.15914 | 0 | 0 | 0 | 0.065345 | 0 | 0 | −0.18105 | 0.88771 |
| 2898 | −0.19824 | 0 | 0 | 0 | 0.04766 | 0 | 0 | 0 | 0.888572 |
| 2899 | −0.19247 | 0 | 0 | 0 | 0.035851 | 0 | 0 | 0 | 0.891841 |
| 2900 | −0.20776 | 0 | 0 | 0 | 0.041076 | 0 | 0 | 0 | 0.885277 |
| 2901 | −0.2108 | 0 | 0 | 0 | 0.018201 | 0 | 0 | 0 | 0.86645 |
| 2902 | −0.20326 | 0 | 0 | 0 | 0.01929 | 0 | 0 | 0 | 0.873431 |
| 2903 | −5.24821 | 1.478394 | 0.065471 | 0.200851 | −0.02152 | 0 | 0 | 0 | 0.550133 |
| 2904 | −5.55543 | 1.371412 | 0.072154 | 0.196383 | −0.01651 | 0 | 0 | 0 | 0.63176 |
| 2905 | −5.43061 | 1.440413 | 0.068922 | 0.180993 | −0.03532 | 0 | 0 | 0 | 0.549656 |
| 2906 | −5.42485 | 1.417363 | 0.068869 | 0.193379 | −0.03434 | 0 | 0 | 0 | 0.541769 |
| 2907 | −5.42759 | 1.440923 | 0.06882 | 0.186555 | −0.04278 | 0 | 0 | 0 | 0.551453 |
| 2908 | −5.28132 | 1.499 | 0.066088 | 0.195754 | 0 | −0.12643 | 0 | 0 | 0.553364 |
| 2909 | −5.59156 | 1.391489 | 0.072782 | 0.191437 | 0 | −0.11895 | 0 | 0 | 0.631524 |
| 2910 | −5.46001 | 1.457103 | 0.069518 | 0.174296 | 0 | −0.12822 | 0 | 0 | 0.552395 |
| 2911 | −5.44221 | 1.43512 | 0.069435 | 0.187073 | 0 | −0.14951 | 0 | 0 | 0.559837 |
| 2912 | −5.45125 | 1.456284 | 0.069352 | 0.178994 | 0 | −0.1303 | 0 | 0 | 0.554815 |
| 2913 | −5.28153 | 1.4725 | 0.065839 | 0.212161 | 0 | 0 | −0.08072 | 0 | 0.542991 |
| 2914 | −5.58285 | 1.367167 | 0.072422 | 0.207211 | 0 | 0 | −0.07983 | 0 | 0.625064 |
| 2915 | −5.45152 | 1.432009 | 0.06911 | 0.192201 | 0 | 0 | −0.08392 | 0 | 0.542561 |
| 2916 | −5.45574 | 1.408747 | 0.069214 | 0.204352 | 0 | 0 | −0.08495 | 0 | 0.533337 |
| 2917 | −5.45144 | 1.429803 | 0.069072 | 0.197212 | 0 | 0 | −0.08857 | 0 | 0.543887 |
| 2918 | −5.36972 | 1.489714 | 0.067817 | 0.216589 | 0 | 0 | 0 | −0.1588 | 0.542851 |
| 2919 | −5.68904 | 1.383997 | 0.075023 | 0.21878 | 0 | 0 | 0 | −0.18557 | 0.659437 |
| 2920 | −5.53047 | 1.449061 | 0.071161 | 0.198249 | 0 | 0 | 0 | −0.17487 | 0.562968 |
| 2921 | −5.52023 | 1.423821 | 0.071153 | 0.213023 | 0 | 0 | 0 | −0.17985 | 0.564277 |
| 2922 | −5.49405 | 1.439232 | 0.071005 | 0.209561 | 0 | 0 | 0 | −0.19953 | 0.601671 |
| 2923 | −5.19494 | 1.456373 | 0.065162 | 0.193458 | 0 | 0 | 0 | 0 | 0.582799 |
| 2924 | −5.26205 | 1.379042 | 0.067642 | 0.227194 | 0 | 0 | 0 | 0 | 0.686825 |
| 2925 | −5.50474 | 1.349008 | 0.071848 | 0.189024 | 0 | 0 | 0 | 0 | 0.654257 |
| 2926 | −5.48757 | 1.327177 | 0.071593 | 0.202193 | 0 | 0 | 0 | 0 | 0.649551 |
| 2927 | −5.49253 | 1.352092 | 0.071535 | 0.193759 | 0 | 0 | 0 | 0 | 0.652821 |
| 2928 | −5.14578 | 1.426629 | 0.064534 | 0.210403 | 0 | 0 | 0 | 0 | 0.581284 |
| 2929 | −5.13536 | 1.453982 | 0.064222 | 0.201666 | 0 | 0 | 0 | 0 | 0.588835 |
| 2930 | −5.35734 | 1.394973 | 0.068371 | 0.185167 | 0 | 0 | 0 | 0 | 0.576215 |
| 2931 | −5.35165 | 1.412634 | 0.068261 | 0.177435 | 0 | 0 | 0 | 0 | 0.58545 |
| 2932 | −5.37548 | 1.402242 | 0.068453 | 0.188853 | 0 | 0 | 0 | 0 | 0.567121 |
| 2933 | −5.15828 | 1.514541 | 0.063691 | 0 | 0.023279 | −0.13522 | 0 | 0 | 0.523468 |
| 2934 | −5.44445 | 1.408546 | 0.070042 | 0 | 0.027286 | −0.129 | 0 | 0 | 0.608811 |
| 2935 | −5.3292 | 1.47297 | 0.067105 | 0 | 0.009576 | −0.13408 | 0 | 0 | 0.538826 |
| 2936 | −5.31231 | 1.454563 | 0.06694 | 0 | 0.015228 | −0.15596 | 0 | 0 | 0.53688 |
| 2937 | −5.31701 | 1.473835 | 0.066841 | 0 | 0.002589 | −0.13499 | 0 | 0 | 0.538015 |
| 2938 | −5.14808 | 1.492727 | 0.06323 | 0 | 0.005246 | 0 | −0.06177 | 0 | 0.511667 |
| 2939 | −5.42653 | 1.38877 | 0.069488 | 0 | 0.010687 | 0 | −0.06282 | 0 | 0.601597 |
| 2940 | −5.30984 | 1.452585 | 0.066491 | 0 | −0.00681 | 0 | −0.06411 | 0 | 0.528454 |
| 2941 | −5.31571 | 1.434847 | 0.066496 | 0 | −0.00693 | 0 | −0.06499 | 0 | 0.508963 |
| 2942 | −5.30496 | 1.453316 | 0.066327 | 0 | −0.01424 | 0 | −0.06865 | 0 | 0.526931 |
| 2943 | −5.21631 | 1.499087 | 0.064913 | 0 | 0.038847 | 0 | 0 | −0.15138 | 0.510098 |
| 2944 | −5.50152 | 1.394932 | 0.071611 | 0 | 0.053265 | 0 | 0 | −0.17754 | 0.632883 |
| 2945 | −5.36678 | 1.459039 | 0.068193 | 0 | 0.028642 | 0 | 0 | −0.16598 | 0.545531 |
| 2946 | −5.35827 | 1.437687 | 0.068084 | 0 | 0.035574 | 0 | 0 | −0.17084 | 0.534552 |
| 2947 | −5.32193 | 1.450879 | 0.067818 | 0 | 0.029297 | 0 | 0 | −0.18816 | 0.576257 |
| 2948 | −5.06719 | 1.476624 | 0.06266 | 0 | −0.01412 | 0 | 0 | 0 | 0.559718 |
| 2949 | −5.10539 | 1.400032 | 0.064608 | 0 | 0.006641 | 0 | 0 | 0 | 0.651377 |
| 2950 | −5.36073 | 1.370454 | 0.06911 | 0 | −0.0112 | 0 | 0 | 0 | 0.63639 |
| 2951 | −5.34352 | 1.35382 | 0.068758 | 0 | −0.0102 | 0 | 0 | 0 | 0.623435 |
| 2952 | −5.34361 | 1.375584 | 0.068691 | 0 | −0.01911 | 0 | 0 | 0 | 0.632898 |
| 2953 | −5.02464 | 1.453338 | 0.061996 | 0 | −0.01569 | 0 | 0 | 0 | 0.546136 |
| 2954 | −5.00209 | 1.477341 | 0.061581 | 0 | −0.02522 | 0 | 0 | 0 | 0.562576 |
| 2955 | −5.22743 | 1.419877 | 0.065842 | 0 | −0.02565 | 0 | 0 | 0 | 0.560056 |
| 2956 | −5.2141 | 1.435824 | 0.065681 | 0 | −0.03689 | 0 | 0 | 0 | 0.577496 |
| 2957 | −5.2423 | 1.428468 | 0.06584 | 0 | −0.02892 | 0 | 0 | 0 | 0.548313 |
| 2958 | −5.19179 | 1.52021 | 0.063972 | 0 | 0 | −0.129 | −0.05788 | 0 | 0.515148 |
| 2959 | −5.47336 | 1.415647 | 0.070237 | 0 | 0 | −0.12179 | −0.05836 | 0 | 0.600952 |
| 2960 | −5.35055 | 1.475895 | 0.067227 | 0 | 0 | −0.12984 | −0.06094 | 0 | 0.532327 |
| 2961 | −5.34141 | 1.459272 | 0.067164 | 0 | 0 | −0.15096 | −0.06174 | 0 | 0.529157 |
| 2962 | −5.33898 | 1.475231 | 0.066992 | 0 | 0 | −0.13197 | −0.06628 | 0 | 0.532612 |
| 2963 | −5.25322 | 1.526123 | 0.065399 | 0 | 0 | −0.09857 | 0 | −0.13374 | 0.511047 |
| 2964 | −5.5412 | 1.422091 | 0.072048 | 0 | 0 | −0.08633 | 0 | −0.15993 | 0.626663 |
| 2965 | −5.39952 | 1.481986 | 0.068648 | 0 | 0 | −0.09637 | 0 | −0.15002 | 0.546578 |
| 2966 | −5.38251 | 1.463739 | 0.068491 | 0 | 0 | −0.11686 | 0 | −0.15103 | 0.545668 |
| 2967 | −5.35357 | 1.474332 | 0.068241 | 0 | 0 | −0.09322 | 0 | −0.17178 | 0.576276 |
| 2968 | −5.10569 | 1.500759 | 0.063388 | 0 | 0 | −0.13856 | 0 | 0 | 0.566201 |
| 2969 | −5.15475 | 1.429446 | 0.065436 | 0 | 0 | −0.13839 | 0 | 0 | 0.655603 |
| 2970 | −5.40303 | 1.393439 | 0.069874 | 0 | 0 | −0.13453 | 0 | 0 | 0.640179 |
| 2971 | −5.37964 | 1.377933 | 0.069569 | 0 | 0 | −0.15688 | 0 | 0 | 0.639898 |
| 2972 | −5.38048 | 1.397018 | 0.069403 | 0 | 0 | −0.13616 | 0 | 0 | 0.637974 |
| 2973 | −5.04085 | 1.479716 | 0.062545 | 0 | 0 | −0.16507 | 0 | 0 | 0.569633 |
| 2974 | −5.0313 | 1.499856 | 0.062207 | 0 | 0 | −0.14431 | 0 | 0 | 0.571047 |
| 2975 | −5.25232 | 1.4408 | 0.066541 | 0 | 0 | −0.16006 | 0 | 0 | 0.57976 |
| 2976 | −5.24401 | 1.453817 | 0.066352 | 0 | 0 | −0.144 | 0 | 0 | 0.584506 |
| 2977 | −5.26346 | 1.448824 | 0.066477 | 0 | 0 | −0.15669 | 0 | 0 | 0.567641 |
| 2978 | −5.25147 | 1.508999 | 0.065178 | 0 | 0 | 0 | −0.05544 | −0.14461 | 0.502718 |
| 2979 | −5.5343 | 1.407941 | 0.071787 | 0 | 0 | 0 | −0.05243 | −0.16828 | 0.623189 |
| 2980 | −5.39009 | 1.4667 | 0.068304 | 0 | 0 | 0 | −0.05648 | −0.15992 | 0.540098 |
| 2981 | −5.38898 | 1.447343 | 0.068281 | 0 | 0 | 0 | −0.05779 | −0.16351 | 0.527438 |
| 2982 | −5.3475 | 1.458758 | 0.067961 | 0 | 0 | 0 | −0.06123 | −0.18156 | 0.571684 |
| 2983 | −5.08801 | 1.475389 | 0.062833 | 0 | 0 | 0 | −0.05851 | 0 | 0.552935 |
| 2984 | −5.13278 | 1.403972 | 0.064862 | 0 | 0 | 0 | −0.04964 | 0 | 0.645734 |
| 2985 | −5.37688 | 1.370342 | 0.069208 | 0 | 0 | 0 | −0.05875 | 0 | 0.630527 |
| 2986 | −5.36537 | 1.354152 | 0.068939 | 0 | 0 | 0 | −0.05939 | 0 | 0.616605 |
| 2987 | −5.36202 | 1.373561 | 0.068833 | 0 | 0 | 0 | −0.06451 | 0 | 0.627039 |
| 2988 | −5.05155 | 1.451908 | 0.062271 | 0 | 0 | 0 | −0.05821 | 0 | 0.5387 |
| 2989 | −5.0239 | 1.473497 | 0.061788 | 0 | 0 | 0 | −0.06282 | 0 | 0.555784 |
| 2990 | −5.24527 | 1.416399 | 0.065976 | 0 | 0 | 0 | −0.06266 | 0 | 0.552056 |
| 2991 | −5.22745 | 1.429627 | 0.065759 | 0 | 0 | 0 | −0.06707 | 0 | 0.569375 |
| 2992 | −5.26319 | 1.423976 | 0.066024 | 0 | 0 | 0 | −0.06749 | 0 | 0.540122 |
| 2993 | −5.16194 | 1.4892 | 0.064567 | 0 | 0 | 0 | 0 | −0.1524 | 0.557272 |
| 2994 | −5.23161 | 1.420421 | 0.067034 | 0 | 0 | 0 | 0 | −0.1619 | 0.659352 |
| 2995 | −5.46753 | 1.384605 | 0.071507 | 0 | 0 | 0 | 0 | −0.17912 | 0.664602 |
| 2996 | −5.44586 | 1.366719 | 0.071143 | 0 | 0 | 0 | 0 | −0.18199 | 0.656271 |
| 2997 | −5.42783 | 1.380137 | 0.071127 | 0 | 0 | 0 | 0 | −0.20257 | 0.690584 |
| 2998 | −5.11043 | 1.46488 | 0.063843 | 0 | 0 | 0 | 0 | −0.15666 | 0.5501 |
| 2999 | −5.05187 | 1.481251 | 0.06315 | 0 | 0 | 0 | 0 | −0.17679 | 0.587989 |
| 3000 | −5.29864 | 1.42853 | 0.067669 | 0 | 0 | 0 | 0 | −0.17212 | 0.581452 |
| 3001 | −5.25608 | 1.43578 | 0.067393 | 0 | 0 | 0 | 0 | −0.1929 | 0.623159 |
| 3002 | −5.28842 | 1.430542 | 0.067554 | 0 | 0 | 0 | 0 | −0.18662 | 0.592113 |
| 3003 | −5.08922 | 1.385169 | 0.064688 | 0 | 0 | 0 | 0 | 0 | 0.667464 |
| 3004 | −5.02891 | 1.358172 | 0.063792 | 0 | 0 | 0 | 0 | 0 | 0.656168 |
| 3005 | −5.01337 | 1.383186 | 0.063436 | 0 | 0 | 0 | 0 | 0 | 0.664618 |
| 3006 | −5.30363 | 1.335981 | 0.068581 | 0 | 0 | 0 | 0 | 0 | 0.646968 |
| 3007 | −5.29528 | 1.351024 | 0.068453 | 0 | 0 | 0 | 0 | 0 | 0.655182 |
| 3008 | −5.30508 | 1.346116 | 0.06837 | 0 | 0 | 0 | 0 | 0 | 0.638858 |
| 3009 | −4.98339 | 1.436048 | 0.061806 | 0 | 0 | 0 | 0 | 0 | 0.576066 |
| 3010 | −4.95515 | 1.453786 | 0.061367 | 0 | 0 | 0 | 0 | 0 | 0.591158 |
| 3011 | −4.9514 | 1.442521 | 0.061136 | 0 | 0 | 0 | 0 | 0 | 0.570176 |
| 3012 | −5.17841 | 1.405567 | 0.065421 | 0 | 0 | 0 | 0 | 0 | 0.583119 |
| 3013 | −0.94563 | 1.239253 | 0 | 0.101967 | 0.012386 | −0.09309 | 0 | 0 | 0.807051 |
| 3014 | −0.94706 | 1.102802 | 0 | 0.065768 | 0.012879 | −0.07809 | 0 | 0 | 0.688555 |
| 3015 | −0.94089 | 1.172042 | 0 | 0.065853 | −0.00678 | −0.0886 | 0 | 0 | 0.734458 |
| 3016 | −0.94652 | 1.157451 | 0 | 0.07865 | −0.0006 | −0.11981 | 0 | 0 | 0.729625 |
| 3017 | −0.96083 | 1.180593 | 0 | 0.065435 | −0.01369 | −0.0918 | 0 | 0 | 0.726514 |
| 3018 | −0.96084 | 1.222084 | 0 | 0.118085 | −0.0012 | 0 | −0.06165 | 0 | 0.797179 |
| 3019 | −0.96005 | 1.088857 | 0 | 0.082894 | 0.002946 | 0 | −0.06752 | 0 | 0.682006 |
| 3020 | −0.95672 | 1.157342 | 0 | 0.08436 | −0.01741 | 0 | −0.07362 | 0 | 0.726805 |
| 3021 | −0.9729 | 1.141428 | 0 | 0.097701 | −0.01964 | 0 | −0.06989 | 0 | 0.706222 |
| 3022 | −0.97696 | 1.164328 | 0 | 0.084755 | −0.02561 | 0 | −0.07613 | 0 | 0.718005 |
| 3023 | −0.92737 | 1.221903 | 0 | 0.113766 | 0.024148 | 0 | 0 | −0.11174 | 0.796644 |
| 3024 | −0.91329 | 1.085419 | 0 | 0.080256 | 0.035039 | 0 | 0 | −0.13253 | 0.696214 |
| 3025 | −0.91336 | 1.156313 | 0 | 0.079897 | 0.010602 | 0 | 0 | −0.12974 | 0.735708 |
| 3026 | −0.92261 | 1.136777 | 0 | 0.094991 | 0.013278 | 0 | 0 | −0.13605 | 0.723872 |
| 3027 | −0.90459 | 1.153572 | 0 | 0.083894 | 0.011349 | 0 | 0 | −0.15839 | 0.757187 |
| 3028 | −0.9233 | 1.204334 | 0 | 0.101549 | −0.02587 | 0 | 0 | 0 | 0.813266 |
| 3029 | −0.88671 | 1.098744 | 0 | 0.118125 | 0.000746 | 0 | 0 | 0 | 0.808522 |
| 3030 | −0.90793 | 1.068854 | 0 | 0.0692 | −0.02096 | 0 | 0 | 0 | 0.720939 |
| 3031 | −0.92134 | 1.058414 | 0 | 0.083937 | −0.02239 | 0 | 0 | 0 | 0.710718 |
| 3032 | −0.92917 | 1.081062 | 0 | 0.070143 | −0.02818 | 0 | 0 | 0 | 0.715326 |
| 3033 | −0.92664 | 1.181589 | 0 | 0.123589 | −0.03065 | 0 | 0 | 0 | 0.808933 |
| 3034 | −0.93297 | 1.212806 | 0 | 0.107283 | −0.0393 | 0 | 0 | 0 | 0.820368 |
| 3035 | −0.92537 | 1.12728 | 0 | 0.083589 | −0.04171 | 0 | 0 | 0 | 0.743015 |
| 3036 | −0.92398 | 1.147198 | 0 | 0.069877 | −0.0525 | 0 | 0 | 0 | 0.757549 |
| 3037 | −0.95148 | 1.142855 | 0 | 0.082422 | −0.04354 | 0 | 0 | 0 | 0.729933 |
| 3038 | −0.95669 | 1.237926 | 0 | 0.113808 | 0 | −0.08766 | −0.05861 | 0 | 0.801624 |
| 3039 | −0.95874 | 1.102737 | 0 | 0.079228 | 0 | −0.07237 | −0.0649 | 0 | 0.683212 |
| 3040 | −0.94906 | 1.167528 | 0 | 0.078281 | 0 | −0.08682 | −0.07235 | 0 | 0.7315 |
| 3041 | −0.95548 | 1.153719 | 0 | 0.090661 | 0 | −0.11714 | −0.06797 | 0 | 0.724799 |
| 3042 | −0.96748 | 1.173442 | 0 | 0.07759 | 0 | −0.09161 | −0.07551 | 0 | 0.72387 |
| 3043 | −0.9306 | 1.238816 | 0 | 0.112755 | 0 | −0.06589 | 0 | −0.09973 | 0.799579 |
| 3044 | −0.92077 | 1.101911 | 0 | 0.081096 | 0 | −0.04779 | 0 | −0.12202 | 0.69522 |
| 3045 | −0.91315 | 1.168297 | 0 | 0.077502 | 0 | −0.062 | 0 | −0.1206 | 0.739091 |
| 3046 | −0.91643 | 1.152297 | 0 | 0.092016 | 0 | −0.09174 | 0 | −0.1229 | 0.737307 |
| 3047 | −0.9053 | 1.166001 | 0 | 0.081661 | 0 | −0.06025 | 0 | −0.14893 | 0.759851 |
| 3048 | −0.91353 | 1.215704 | 0 | 0.094764 | 0 | −0.10081 | 0 | 0 | 0.819418 |
| 3049 | −0.88317 | 1.115259 | 0 | 0.114111 | 0 | −0.09267 | 0 | 0 | 0.812556 |
| 3050 | −0.89999 | 1.077814 | 0 | 0.062923 | 0 | −0.08821 | 0 | 0 | 0.72522 |
| 3051 | −0.9053 | 1.068734 | 0 | 0.076763 | 0 | −0.1158 | 0 | 0 | 0.726029 |
| 3052 | −0.92003 | 1.089057 | 0 | 0.062653 | 0 | −0.09136 | 0 | 0 | 0.719929 |
| 3053 | −0.90569 | 1.196767 | 0 | 0.116209 | 0 | −0.14137 | 0 | 0 | 0.830587 |
| 3054 | −0.92023 | 1.223004 | 0 | 0.098755 | 0 | −0.11167 | 0 | 0 | 0.82809 |
| 3055 | −0.90366 | 1.134708 | 0 | 0.074053 | 0 | −0.1279 | 0 | 0 | 0.760379 |
| 3056 | −0.90861 | 1.15124 | 0 | 0.059436 | 0 | −0.10779 | 0 | 0 | 0.76336 |
| 3057 | −0.93075 | 1.150344 | 0 | 0.072663 | 0 | −0.1245 | 0 | 0 | 0.746976 |
| 3058 | −0.94099 | 1.224515 | 0 | 0.126646 | 0 | 0 | −0.05861 | −0.10687 | 0.791795 |
| 3059 | −0.93004 | 1.091985 | 0 | 0.095326 | 0 | 0 | −0.06248 | −0.12564 | 0.690176 |
| 3060 | −0.92451 | 1.156891 | 0 | 0.094039 | 0 | 0 | −0.0712 | −0.12627 | 0.733939 |
| 3061 | −0.93407 | 1.137285 | 0 | 0.108523 | 0 | 0 | −0.06728 | −0.13214 | 0.720083 |
| 3062 | −0.91544 | 1.153205 | 0 | 0.098805 | 0 | 0 | −0.07423 | −0.15486 | 0.756127 |
| 3063 | −0.92893 | 1.195478 | 0 | 0.110377 | 0 | 0 | −0.06451 | 0 | 0.808242 |
| 3064 | −0.89464 | 1.096875 | 0 | 0.126457 | 0 | 0 | −0.04788 | 0 | 0.803488 |
| 3065 | −0.91379 | 1.061455 | 0 | 0.079594 | 0 | 0 | −0.06971 | 0 | 0.716912 |
| 3066 | −0.92678 | 1.050235 | 0 | 0.093031 | 0 | 0 | −0.06683 | 0 | 0.705246 |
| 3067 | −0.93398 | 1.071002 | 0 | 0.079938 | 0 | 0 | −0.07218 | 0 | 0.710574 |
| 3068 | −0.93089 | 1.170886 | 0 | 0.130164 | 0 | 0 | −0.05836 | 0 | 0.802184 |
| 3069 | −0.93661 | 1.199889 | 0 | 0.114976 | 0 | 0 | −0.06732 | 0 | 0.814656 |
| 3070 | −0.92941 | 1.114537 | 0 | 0.092126 | 0 | 0 | −0.07447 | 0 | 0.736698 |
| 3071 | −0.92702 | 1.13158 | 0 | 0.078733 | 0 | 0 | −0.08076 | 0 | 0.751004 |
| 3072 | −0.95514 | 1.128749 | 0 | 0.090768 | 0 | 0 | −0.07573 | 0 | 0.723026 |
| 3073 | −0.89598 | 1.201803 | 0 | 0.108979 | 0 | 0 | 0 | −0.11801 | 0.812196 |
| 3074 | −0.86174 | 1.100703 | 0 | 0.12987 | 0 | 0 | 0 | −0.11871 | 0.809983 |
| 3075 | −0.86901 | 1.064779 | 0 | 0.080877 | 0 | 0 | 0 | −0.13681 | 0.734949 |
| 3076 | −0.87646 | 1.051244 | 0 | 0.096862 | 0 | 0 | 0 | −0.14165 | 0.731041 |
| 3077 | −0.86418 | 1.06598 | 0 | 0.085804 | 0 | 0 | 0 | −0.16202 | 0.755867 |
| 3078 | −0.89165 | 1.17576 | 0 | 0.13249 | 0 | 0 | 0 | −0.12703 | 0.815453 |
| 3079 | −0.87561 | 1.199093 | 0 | 0.12 | 0 | 0 | 0 | −0.1558 | 0.847748 |
| 3080 | −0.87998 | 1.117849 | 0 | 0.093325 | 0 | 0 | 0 | −0.14212 | 0.759505 |
| 3081 | −0.85497 | 1.129296 | 0 | 0.082213 | 0 | 0 | 0 | −0.167 | 0.796144 |
| 3082 | −0.88679 | 1.125724 | 0 | 0.095581 | 0 | 0 | 0 | −0.16051 | 0.767107 |
| 3083 | −0.8616 | 1.07863 | 0 | 0.109375 | 0 | 0 | 0 | 0 | 0.81445 |
| 3084 | −0.85743 | 1.054396 | 0 | 0.130531 | 0 | 0 | 0 | 0 | 0.818646 |
| 3085 | −0.86725 | 1.081993 | 0 | 0.113562 | 0 | 0 | 0 | 0 | 0.82137 |
| 3086 | −0.88112 | 1.030958 | 0 | 0.076073 | 0 | 0 | 0 | 0 | 0.734034 |
| 3087 | −0.88153 | 1.046829 | 0 | 0.06215 | 0 | 0 | 0 | 0 | 0.742183 |
| 3088 | −0.90678 | 1.048177 | 0 | 0.075759 | 0 | 0 | 0 | 0 | 0.723439 |
| 3089 | −0.89683 | 1.155201 | 0 | 0.111369 | 0 | 0 | 0 | 0 | 0.815853 |
| 3090 | −0.89761 | 1.178922 | 0 | 0.094616 | 0 | 0 | 0 | 0 | 0.827269 |
| 3091 | −0.9107 | 1.168952 | 0 | 0.113928 | 0 | 0 | 0 | 0 | 0.818266 |
| 3092 | −0.90616 | 1.110244 | 0 | 0.072386 | 0 | 0 | 0 | 0 | 0.754057 |
| 3093 | −0.97271 | 1.248758 | 0 | 0 | 0.032175 | −0.09891 | −0.04839 | 0 | 0.781395 |
| 3094 | −0.9655 | 1.109464 | 0 | 0 | 0.030087 | −0.08239 | −0.05772 | 0 | 0.674752 |
| 3095 | −0.95948 | 1.178018 | 0 | 0 | 0.011708 | −0.09271 | −0.06326 | 0 | 0.722547 |
| 3096 | −0.96814 | 1.165243 | 0 | 0 | 0.018411 | −0.1243 | −0.05852 | 0 | 0.711474 |
| 3097 | −0.97839 | 1.185431 | 0 | 0 | 0.004941 | −0.096 | −0.06623 | 0 | 0.715514 |
| 3098 | −0.94434 | 1.244241 | 0 | 0 | 0.051882 | −0.08029 | 0 | −0.10063 | 0.778892 |
| 3099 | −0.92323 | 1.102599 | 0 | 0 | 0.056461 | −0.06209 | 0 | −0.12428 | 0.68694 |
| 3100 | −0.92111 | 1.173438 | 0 | 0 | 0.033451 | −0.07187 | 0 | −0.12039 | 0.729094 |
| 3101 | −0.92601 | 1.157248 | 0 | 0 | 0.04448 | −0.10377 | 0 | −0.12358 | 0.722729 |
| 3102 | −0.91366 | 1.171307 | 0 | 0 | 0.034928 | −0.07083 | 0 | −0.14813 | 0.748853 |
| 3103 | −0.9306 | 1.228622 | 0 | 0 | 0.009489 | −0.10612 | 0 | 0 | 0.804132 |
| 3104 | −0.8978 | 1.12548 | 0 | 0 | 0.038103 | −0.10533 | 0 | 0 | 0.794165 |
| 3105 | −0.90887 | 1.086879 | 0 | 0 | 0.008271 | −0.09249 | 0 | 0 | 0.718879 |
| 3106 | −0.91638 | 1.078827 | 0 | 0 | 0.015909 | −0.12188 | 0 | 0 | 0.715508 |
| 3107 | −0.92962 | 1.099287 | 0 | 0 | 0.002268 | −0.09446 | 0 | 0 | 0.713698 |
| 3108 | −0.92742 | 1.211939 | 0 | 0 | 0.016803 | −0.14777 | 0 | 0 | 0.80731 |
| 3109 | −0.93955 | 1.238886 | 0 | 0 | −0.00099 | −0.1147 | 0 | 0 | 0.812113 |
| 3110 | −0.91769 | 1.148347 | 0 | 0 | −0.00295 | −0.12976 | 0 | 0 | 0.750996 |
| 3111 | −0.92094 | 1.165946 | 0 | 0 | −0.02033 | −0.10575 | 0 | 0 | 0.760028 |
| 3112 | −0.94453 | 1.163934 | 0 | 0 | −0.00499 | −0.12588 | 0 | 0 | 0.737851 |
| 3113 | −0.95763 | 1.231516 | 0 | 0 | 0.043548 | 0 | −0.04777 | −0.10814 | 0.76858 |
| 3114 | −0.93502 | 1.093854 | 0 | 0 | 0.05217 | 0 | −0.05527 | −0.12861 | 0.680242 |
| 3115 | −0.93517 | 1.163833 | 0 | 0 | 0.029284 | 0 | −0.06168 | −0.12617 | 0.721789 |
| 3116 | −0.94892 | 1.146355 | 0 | 0 | 0.0323 | 0 | −0.0566 | −0.13185 | 0.701658 |
| 3117 | −0.92694 | 1.160878 | 0 | 0 | 0.030016 | 0 | −0.06436 | −0.15388 | 0.742615 |
| 3118 | −0.9491 | 1.213461 | 0 | 0 | −0.00581 | 0 | −0.05013 | 0 | 0.791436 |
| 3119 | −0.91348 | 1.112135 | 0 | 0 | 0.0195 | 0 | −0.03573 | 0 | 0.783029 |
| 3120 | −0.92591 | 1.075528 | 0 | 0 | −0.00297 | 0 | −0.05878 | 0 | 0.709341 |
| 3121 | −0.94298 | 1.06768 | 0 | 0 | −0.00358 | 0 | −0.05493 | 0 | 0.692728 |
| 3122 | −0.9469 | 1.087056 | 0 | 0 | −0.01015 | 0 | −0.06107 | 0 | 0.703333 |
| 3123 | −0.95742 | 1.194035 | 0 | 0 | −0.0093 | 0 | −0.04258 | 0 | 0.777359 |
| 3124 | −0.95923 | 1.222298 | 0 | 0 | −0.01842 | 0 | −0.05202 | 0 | 0.797578 |
| 3125 | −0.94738 | 1.135526 | 0 | 0 | −0.02192 | 0 | −0.06005 | 0 | 0.726415 |
| 3126 | −0.94187 | 1.15274 | 0 | 0 | −0.0333 | 0 | −0.06676 | 0 | 0.747648 |
| 3127 | −0.97285 | 1.149935 | 0 | 0 | −0.0235 | 0 | −0.06208 | 0 | 0.713213 |
| 3128 | −0.91443 | 1.212134 | 0 | 0 | 0.020475 | 0 | 0 | −0.11606 | 0.793867 |
| 3129 | −0.87737 | 1.107735 | 0 | 0 | 0.051348 | 0 | 0 | −0.11972 | 0.788587 |
| 3130 | −0.87773 | 1.070732 | 0 | 0 | 0.028796 | 0 | 0 | −0.13655 | 0.72584 |
| 3131 | −0.88929 | 1.059257 | 0 | 0 | 0.03351 | 0 | 0 | −0.1414 | 0.716019 |
| 3132 | −0.87345 | 1.072048 | 0 | 0 | 0.030887 | 0 | 0 | −0.16142 | 0.745376 |
| 3133 | −0.91726 | 1.189383 | 0 | 0 | 0.022474 | 0 | 0 | −0.12403 | 0.786861 |
| 3134 | −0.89751 | 1.211855 | 0 | 0 | 0.018368 | 0 | 0 | −0.15165 | 0.825994 |
| 3135 | −0.89674 | 1.130014 | 0 | 0 | 0.010937 | 0 | 0 | −0.13886 | 0.745211 |
| 3136 | −0.86934 | 1.141413 | 0 | 0 | 0.003008 | 0 | 0 | −0.16261 | 0.786859 |
| 3137 | −0.90348 | 1.137232 | 0 | 0 | 0.014832 | 0 | 0 | −0.15705 | 0.751513 |
| 3138 | −0.88249 | 1.09655 | 0 | 0 | −0.00085 | 0 | 0 | 0 | 0.797376 |
| 3139 | −0.88488 | 1.07829 | 0 | 0 | −0.00524 | 0 | 0 | 0 | 0.79398 |
| 3140 | −0.89055 | 1.104228 | 0 | 0 | −0.01418 | 0 | 0 | 0 | 0.803945 |
| 3141 | −0.89819 | 1.050751 | 0 | 0 | −0.02459 | 0 | 0 | 0 | 0.726327 |
| 3142 | −0.89593 | 1.066528 | 0 | 0 | −0.03614 | 0 | 0 | 0 | 0.740287 |
| 3143 | −0.92376 | 1.067955 | 0 | 0 | −0.02591 | 0 | 0 | 0 | 0.715651 |
| 3144 | −0.92351 | 1.180178 | 0 | 0 | −0.02882 | 0 | 0 | 0 | 0.797828 |
| 3145 | −0.92071 | 1.204429 | 0 | 0 | −0.04311 | 0 | 0 | 0 | 0.818139 |
| 3146 | −0.9387 | 1.195655 | 0 | 0 | −0.03455 | 0 | 0 | 0 | 0.799718 |
| 3147 | −0.92409 | 1.134635 | 0 | 0 | −0.0484 | 0 | 0 | 0 | 0.751829 |
| 3148 | −0.96364 | 1.254072 | 0 | 0 | 0 | −0.07036 | −0.04257 | −0.09315 | 0.772091 |
| 3149 | −0.94565 | 1.115617 | 0 | 0 | 0 | −0.05121 | −0.04971 | −0.11544 | 0.678828 |
| 3150 | −0.93727 | 1.180452 | 0 | 0 | 0 | −0.06464 | −0.05758 | −0.11445 | 0.726259 |
| 3151 | −0.94483 | 1.166879 | 0 | 0 | 0 | −0.09488 | −0.05242 | −0.11595 | 0.716812 |
| 3152 | −0.93034 | 1.17837 | 0 | 0 | 0 | −0.06346 | −0.06079 | −0.14162 | 0.746275 |
| 3153 | −0.94108 | 1.229432 | 0 | 0 | 0 | −0.10218 | −0.04773 | 0 | 0.799199 |
| 3154 | −0.91198 | 1.13394 | 0 | 0 | 0 | −0.09583 | −0.03048 | 0 | 0.787755 |
| 3155 | −0.91979 | 1.088297 | 0 | 0 | 0 | −0.08851 | −0.05671 | 0 | 0.714558 |
| 3156 | −0.92832 | 1.081967 | 0 | 0 | 0 | −0.11667 | −0.05247 | 0 | 0.709798 |
| 3157 | −0.93937 | 1.098723 | 0 | 0 | 0 | −0.09182 | −0.05983 | 0 | 0.709473 |
| 3158 | −0.93798 | 1.21442 | 0 | 0 | 0 | −0.1427 | −0.03928 | 0 | 0.801606 |
| 3159 | −0.94805 | 1.23696 | 0 | 0 | 0 | −0.11299 | −0.05068 | 0 | 0.807662 |
| 3160 | −0.92655 | 1.146715 | 0 | 0 | 0 | −0.1283 | −0.05924 | 0 | 0.746565 |
| 3161 | −0.92741 | 1.160009 | 0 | 0 | 0 | −0.10768 | −0.06767 | 0 | 0.756284 |
| 3162 | −0.95313 | 1.161199 | 0 | 0 | 0 | −0.12498 | −0.06163 | 0 | 0.733262 |
| 3163 | −0.91475 | 1.229484 | 0 | 0 | 0 | −0.0791 | 0 | −0.10298 | 0.799712 |
| 3164 | −0.88695 | 1.132346 | 0 | 0 | 0 | −0.0724 | 0 | −0.10332 | 0.789433 |
| 3165 | −0.88122 | 1.086583 | 0 | 0 | 0 | −0.06298 | 0 | −0.12526 | 0.728634 |
| 3166 | −0.88823 | 1.078484 | 0 | 0 | 0 | −0.09088 | 0 | −0.12591 | 0.726591 |
| 3167 | −0.87859 | 1.08901 | 0 | 0 | 0 | −0.06066 | 0 | −0.14942 | 0.747046 |
| 3168 | −0.91022 | 1.212426 | 0 | 0 | 0 | −0.1183 | 0 | −0.10528 | 0.805254 |
| 3169 | −0.89812 | 1.229783 | 0 | 0 | 0 | −0.08142 | 0 | −0.13793 | 0.831308 |
| 3170 | −0.88759 | 1.144711 | 0 | 0 | 0 | −0.10217 | 0 | −0.12504 | 0.760386 |
| 3171 | −0.86561 | 1.152397 | 0 | 0 | 0 | −0.07547 | 0 | −0.15265 | 0.792224 |
| 3172 | −0.89737 | 1.153244 | 0 | 0 | 0 | −0.09546 | 0 | −0.14333 | 0.764763 |
| 3173 | −0.87563 | 1.113547 | 0 | 0 | 0 | −0.10694 | 0 | 0 | 0.804861 |
| 3174 | −0.86774 | 1.098468 | 0 | 0 | 0 | −0.14614 | 0 | 0 | 0.814968 |
| 3175 | −0.88062 | 1.119495 | 0 | 0 | 0 | −0.11684 | 0 | 0 | 0.813344 |
| 3176 | −0.87829 | 1.059473 | 0 | 0 | 0 | −0.12669 | 0 | 0 | 0.743966 |
| 3177 | −0.88121 | 1.072046 | 0 | 0 | 0 | −0.1075 | 0 | 0 | 0.748248 |
| 3178 | −0.90533 | 1.076985 | 0 | 0 | 0 | −0.12264 | 0 | 0 | 0.732459 |
| 3179 | −0.90038 | 1.195351 | 0 | 0 | 0 | −0.14866 | 0 | 0 | 0.820628 |
| 3180 | −0.90379 | 1.213686 | 0 | 0 | 0 | −0.12469 | 0 | 0 | 0.828264 |
| 3181 | −0.91568 | 1.210441 | 0 | 0 | 0 | −0.14966 | 0 | 0 | 0.822573 |
| 3182 | −0.89939 | 1.139851 | 0 | 0 | 0 | −0.13537 | 0 | 0 | 0.769759 |
| 3183 | −0.9269 | 1.216441 | 0 | 0 | 0 | 0 | −0.04604 | −0.11152 | 0.789959 |
| 3184 | −0.89466 | 1.120619 | 0 | 0 | 0 | 0 | −0.02809 | −0.11129 | 0.782353 |
| 3185 | −0.89265 | 1.078079 | 0 | 0 | 0 | 0 | −0.0521 | −0.13024 | 0.721889 |
| 3186 | −0.90483 | 1.067713 | 0 | 0 | 0 | 0 | −0.0481 | −0.13417 | 0.710525 |
| 3187 | −0.88917 | 1.079809 | 0 | 0 | 0 | 0 | −0.05446 | −0.1544 | 0.741081 |
| 3188 | −0.92892 | 1.194148 | 0 | 0 | 0 | 0 | −0.03857 | −0.11926 | 0.782227 |
| 3189 | −0.90958 | 1.215539 | 0 | 0 | 0 | 0 | −0.04801 | −0.1472 | 0.822517 |
| 3190 | −0.90785 | 1.132518 | 0 | 0 | 0 | 0 | −0.05624 | −0.13502 | 0.742024 |
| 3191 | −0.87921 | 1.141922 | 0 | 0 | 0 | 0 | −0.0639 | −0.15979 | 0.785416 |
| 3192 | −0.91555 | 1.140255 | 0 | 0 | 0 | 0 | −0.05775 | −0.15251 | 0.748096 |
| 3193 | −0.88938 | 1.096109 | 0 | 0 | 0 | 0 | −0.03624 | 0 | 0.793004 |
| 3194 | −0.88977 | 1.076622 | 0 | 0 | 0 | 0 | −0.03052 | 0 | 0.789869 |
| 3195 | −0.89533 | 1.100065 | 0 | 0 | 0 | 0 | −0.03915 | 0 | 0.799471 |
| 3196 | −0.90347 | 1.044066 | 0 | 0 | 0 | 0 | −0.05809 | 0 | 0.720965 |
| 3197 | −0.90024 | 1.056673 | 0 | 0 | 0 | 0 | −0.06471 | 0 | 0.73446 |
| 3198 | −0.92903 | 1.060497 | 0 | 0 | 0 | 0 | −0.05945 | 0 | 0.709586 |
| 3199 | −0.92774 | 1.171954 | 0 | 0 | 0 | 0 | −0.04663 | 0 | 0.791154 |
| 3200 | −0.92413 | 1.192585 | 0 | 0 | 0 | 0 | −0.05627 | 0 | 0.810721 |
| 3201 | −0.94203 | 1.185665 | 0 | 0 | 0 | 0 | −0.04863 | 0 | 0.792674 |
| 3202 | −0.928 | 1.121575 | 0 | 0 | 0 | 0 | −0.06729 | 0 | 0.743389 |
| 3203 | −0.85756 | 1.099651 | 0 | 0 | 0 | 0 | 0 | −0.12075 | 0.800384 |
| 3204 | −0.85336 | 1.079152 | 0 | 0 | 0 | 0 | 0 | −0.12861 | 0.803555 |
| 3205 | −0.8414 | 1.09686 | 0 | 0 | 0 | 0 | 0 | −0.15443 | 0.828509 |
| 3206 | −0.85343 | 1.044795 | 0 | 0 | 0 | 0 | 0 | −0.14411 | 0.747291 |
| 3207 | −0.82939 | 1.051175 | 0 | 0 | 0 | 0 | 0 | −0.16718 | 0.78095 |
| 3208 | −0.86317 | 1.055353 | 0 | 0 | 0 | 0 | 0 | −0.15943 | 0.753513 |
| 3209 | −0.88972 | 1.176255 | 0 | 0 | 0 | 0 | 0 | −0.12818 | 0.80473 |
| 3210 | −0.86443 | 1.192154 | 0 | 0 | 0 | 0 | 0 | −0.15765 | 0.844037 |
| 3211 | −0.88603 | 1.184162 | 0 | 0 | 0 | 0 | 0 | −0.15248 | 0.824823 |
| 3212 | −0.8594 | 1.118631 | 0 | 0 | 0 | 0 | 0 | −0.16435 | 0.788041 |
| 3213 | −0.86211 | 1.061783 | 0 | 0 | 0 | 0 | 0 | 0 | 0.801907 |
| 3214 | −0.86146 | 1.080173 | 0 | 0 | 0 | 0 | 0 | 0 | 0.812762 |
| 3215 | −0.87347 | 1.072869 | 0 | 0 | 0 | 0 | 0 | 0 | 0.804105 |
| 3216 | −0.88096 | 1.038263 | 0 | 0 | 0 | 0 | 0 | 0 | 0.739972 |
| 3217 | −0.90515 | 1.167335 | 0 | 0 | 0 | 0 | 0 | 0 | 0.810528 |
| 3218 | −0.20127 | 0 | 0 | 0 | 0 | −0.03476 | −0.04792 | −0.10447 | 0.846351 |
| 3219 | −0.2007 | 0 | 0 | 0 | 0 | −0.03219 | −0.02872 | −0.10667 | 0.866787 |
| 3220 | −0.19404 | 0 | 0 | 0 | 0 | −0.03043 | −0.05106 | −0.1268 | 0.860236 |
| 3221 | −0.20475 | 0 | 0 | 0 | 0 | −0.06385 | −0.04779 | −0.12906 | 0.860405 |
| 3222 | −0.19399 | 0 | 0 | 0 | 0 | −0.02688 | −0.05484 | −0.15867 | 0.873999 |
| 3223 | −0.2008 | 0 | 0 | 0 | 0 | −0.07756 | −0.04138 | −0.10935 | 0.860709 |
| 3224 | −0.18567 | 0 | 0 | 0 | 0 | −0.03684 | −0.05097 | −0.14875 | 0.876561 |
| 3225 | −0.1856 | 0 | 0 | 0 | 0 | −0.07228 | −0.05549 | −0.1275 | 0.866774 |
| 3226 | −0.16497 | 0 | 0 | 0 | 0 | −0.04095 | −0.06346 | −0.1615 | 0.890199 |
| 3227 | −0.19221 | 0 | 0 | 0 | 0 | −0.06358 | −0.05906 | −0.15258 | 0.870267 |
| 3228 | −0.19831 | 0 | 0 | 0 | 0 | −0.06661 | −0.03459 | 0 | 0.885293 |
| 3229 | −0.19841 | 0 | 0 | 0 | 0 | −0.10998 | −0.02796 | 0 | 0.894822 |
| 3230 | −0.20539 | 0 | 0 | 0 | 0 | −0.07605 | −0.0376 | 0 | 0.886476 |
| 3231 | −0.20603 | 0 | 0 | 0 | 0 | −0.09899 | −0.05632 | 0 | 0.875729 |
| 3232 | −0.20752 | 0 | 0 | 0 | 0 | −0.07464 | −0.06367 | 0 | 0.872449 |
| 3233 | −0.22729 | 0 | 0 | 0 | 0 | −0.09252 | −0.05901 | 0 | 0.860456 |
| 3234 | −0.1975 | 0 | 0 | 0 | 0 | −0.10736 | −0.0468 | 0 | 0.881312 |
| 3235 | −0.1984 | 0 | 0 | 0 | 0 | −0.08 | −0.05694 | 0 | 0.878166 |
| 3236 | −0.20935 | 0 | 0 | 0 | 0 | −0.10669 | −0.05035 | 0 | 0.876113 |
| 3237 | −0.20351 | 0 | 0 | 0 | 0 | −0.10236 | −0.06657 | 0 | 0.872583 |
| 3238 | −0.16985 | 0 | 0 | 0 | 0 | −0.04405 | 0 | −0.11507 | 0.889738 |
| 3239 | −0.16943 | 0 | 0 | 0 | 0 | −0.08594 | 0 | −0.11869 | 0.901876 |
| 3240 | −0.15901 | 0 | 0 | 0 | 0 | −0.04476 | 0 | −0.15601 | 0.911737 |
| 3241 | −0.16389 | 0 | 0 | 0 | 0 | −0.07494 | 0 | −0.13887 | 0.893625 |
| 3242 | −0.14642 | 0 | 0 | 0 | 0 | −0.04465 | 0 | −0.17096 | 0.911889 |
| 3243 | −0.17197 | 0 | 0 | 0 | 0 | −0.06499 | 0 | −0.16035 | 0.895672 |
| 3244 | −0.16726 | 0 | 0 | 0 | 0 | −0.08426 | 0 | −0.11687 | 0.885952 |
| 3245 | −0.14686 | 0 | 0 | 0 | 0 | −0.04908 | 0 | −0.15706 | 0.904995 |
| 3246 | −0.16316 | 0 | 0 | 0 | 0 | −0.07791 | 0 | −0.14904 | 0.900285 |
| 3247 | −0.14604 | 0 | 0 | 0 | 0 | −0.07409 | 0 | −0.16345 | 0.907288 |
| 3248 | −0.17554 | 0 | 0 | 0 | 0 | −0.11777 | 0 | 0 | 0.909766 |
| 3249 | −0.17716 | 0 | 0 | 0 | 0 | −0.0905 | 0 | 0 | 0.905179 |
| 3250 | −0.18541 | 0 | 0 | 0 | 0 | −0.11621 | 0 | 0 | 0.906443 |
| 3251 | −0.18843 | 0 | 0 | 0 | 0 | −0.10624 | 0 | 0 | 0.888997 |
| 3252 | −0.17767 | 0 | 0 | 0 | 0 | −0.11562 | 0 | 0 | 0.897882 |
| 3253 | −0.18204 | 0 | 0 | 0 | 0 | 0 | −0.0303 | −0.11879 | 0.882644 |
| 3254 | −0.18892 | 0 | 0 | 0 | 0 | 0 | −0.02491 | −0.12761 | 0.886181 |
| 3255 | −0.17135 | 0 | 0 | 0 | 0 | 0 | −0.03194 | −0.1601 | 0.904984 |
| 3256 | −0.18516 | 0 | 0 | 0 | 0 | 0 | −0.05036 | −0.14401 | 0.878382 |
| 3257 | −0.16177 | 0 | 0 | 0 | 0 | 0 | −0.05631 | −0.1731 | 0.90497 |
| 3258 | −0.1918 | 0 | 0 | 0 | 0 | 0 | −0.05232 | −0.16458 | 0.882117 |
| 3259 | −0.19127 | 0 | 0 | 0 | 0 | 0 | −0.04392 | −0.12511 | 0.867688 |
| 3260 | −0.16336 | 0 | 0 | 0 | 0 | 0 | −0.05181 | −0.16118 | 0.897464 |
| 3261 | −0.18596 | 0 | 0 | 0 | 0 | 0 | −0.04629 | −0.1566 | 0.884089 |
| 3262 | −0.16935 | 0 | 0 | 0 | 0 | 0 | −0.06084 | −0.16883 | 0.892472 |
| 3263 | −0.20529 | 0 | 0 | 0 | 0 | 0 | −0.03306 | 0 | 0.886802 |
| 3264 | −0.2005 | 0 | 0 | 0 | 0 | 0 | −0.04132 | 0 | 0.890409 |
| 3265 | −0.21493 | 0 | 0 | 0 | 0 | 0 | −0.03536 | 0 | 0.88362 |
| 3266 | −0.22005 | 0 | 0 | 0 | 0 | 0 | −0.063 | 0 | 0.865907 |
| 3267 | −0.21241 | 0 | 0 | 0 | 0 | 0 | −0.05392 | 0 | 0.871873 |
| 3268 | −0.16555 | 0 | 0 | 0 | 0 | 0 | 0 | −0.13504 | 0.901725 |
| 3269 | −0.1431 | 0 | 0 | 0 | 0 | 0 | 0 | −0.1699 | 0.923899 |
| 3270 | −0.16099 | 0 | 0 | 0 | 0 | 0 | 0 | −0.16386 | 0.915836 |
| 3271 | −0.15155 | 0 | 0 | 0 | 0 | 0 | 0 | −0.1776 | 0.912672 |
| 3272 | −0.15323 | 0 | 0 | 0 | 0 | 0 | 0 | −0.16561 | 0.906823 |
| 3273 | −6.23446 | 1.54497 | 0.076754 | 0.184711 | 0.040916 | −0.08973 | −0.091 | −0.15739 | 0 |
| 3274 | −0.19249 | 0 | 0 | 0 | 0 | 0 | 0 | 0 | 0.897088 |
| 3275 | −3.61125 | 0 | 0.053483 | 0.229258 | 0.059078 | 0 | 0 | 0 | 0.631612 |
| 3276 | −4.02438 | 0 | 0.061659 | 0.225986 | 0.05721 | 0 | 0 | 0 | 0.790038 |
| 3277 | −3.79716 | 0 | 0.05715 | 0.207984 | 0.041388 | 0 | 0 | 0 | 0.679428 |
| 3278 | −3.83818 | 0 | 0.057731 | 0.225844 | 0.040861 | 0 | 0 | 0 | 0.690953 |
| 3279 | −3.82726 | 0 | 0.057513 | 0.212478 | 0.033178 | 0 | 0 | 0 | 0.67628 |
| 3280 | −3.61906 | 0 | 0.053723 | 0.233066 | 0 | −0.06199 | 0 | 0 | 0.635469 |
| 3281 | −4.0347 | 0 | 0.06193 | 0.229613 | 0 | −0.06238 | 0 | 0 | 0.793002 |
| 3282 | −3.80203 | 0 | 0.057394 | 0.210315 | 0 | −0.07012 | 0 | 0 | 0.687285 |
| 3283 | −3.83006 | 0 | 0.057934 | 0.228406 | 0 | −0.09958 | 0 | 0 | 0.711364 |
| 3284 | −3.82666 | 0 | 0.057706 | 0.214082 | 0 | −0.07252 | 0 | 0 | 0.68645 |
| 3285 | −3.65202 | 0 | 0.053886 | 0.25149 | 0 | 0 | −0.08611 | 0 | 0.626809 |
| 3286 | −4.06017 | 0 | 0.061983 | 0.247092 | 0 | 0 | −0.08298 | 0 | 0.784956 |
| 3287 | −3.82666 | 0 | 0.057396 | 0.228461 | 0 | 0 | −0.08708 | 0 | 0.67726 |
| 3288 | −3.87314 | 0 | 0.058066 | 0.247633 | 0 | 0 | −0.08932 | 0 | 0.688011 |
| 3289 | −3.85837 | 0 | 0.057793 | 0.235108 | 0 | 0 | −0.09456 | 0 | 0.676677 |
| 3290 | −3.69742 | 0 | 0.055241 | 0.247105 | 0 | 0 | 0 | −0.14617 | 0.62007 |
| 3291 | −4.12082 | 0 | 0.063923 | 0.252403 | 0 | 0 | 0 | −0.17571 | 0.810907 |
| 3292 | −3.86497 | 0 | 0.058846 | 0.227785 | 0 | 0 | 0 | −0.16364 | 0.689055 |
| 3293 | −3.89786 | 0 | 0.05941 | 0.248225 | 0 | 0 | 0 | −0.17112 | 0.710766 |
| 3294 | −3.86708 | 0 | 0.059232 | 0.240494 | 0 | 0 | 0 | −0.19969 | 0.729543 |
| 3295 | −3.54861 | 0 | 0.052935 | 0.226346 | 0 | 0 | 0 | 0 | 0.674179 |
| 3296 | −3.67605 | 0 | 0.05585 | 0.260372 | 0 | 0 | 0 | 0 | 0.790851 |
| 3297 | −3.97718 | 0 | 0.061222 | 0.221614 | 0 | 0 | 0 | 0 | 0.808571 |
| 3298 | −4.00933 | 0 | 0.061563 | 0.240199 | 0 | 0 | 0 | 0 | 0.812509 |
| 3299 | −3.98811 | 0 | 0.06123 | 0.225536 | 0 | 0 | 0 | 0 | 0.802245 |
| 3300 | −3.53129 | 0 | 0.052685 | 0.24936 | 0 | 0 | 0 | 0 | 0.69656 |
| 3301 | −3.5113 | 0 | 0.052293 | 0.23356 | 0 | 0 | 0 | 0 | 0.677618 |
| 3302 | −3.76648 | 0 | 0.056993 | 0.221104 | 0 | 0 | 0 | 0 | 0.724207 |
| 3303 | −3.74897 | 0 | 0.056744 | 0.207685 | 0 | 0 | 0 | 0 | 0.716331 |
| 3304 | −3.80736 | 0 | 0.05744 | 0.224637 | 0 | 0 | 0 | 0 | 0.711406 |
| 3305 | −3.44397 | 0 | 0.050648 | 0 | 0.096955 | −0.08417 | 0 | 0 | 0.60354 |
| 3306 | −3.82372 | 0 | 0.058368 | 0 | 0.096906 | −0.08615 | 0 | 0 | 0.770411 |
| 3307 | −3.62218 | 0 | 0.054358 | 0 | 0.080054 | −0.08896 | 0 | 0 | 0.670994 |
| 3308 | −3.637 | 0 | 0.05461 | 0 | 0.087887 | −0.11998 | 0 | 0 | 0.684985 |
| 3309 | −3.643 | 0 | 0.054574 | 0 | 0.073261 | −0.09114 | 0 | 0 | 0.666352 |
| 3310 | −3.45771 | 0 | 0.050471 | 0 | 0.08785 | 0 | −0.06529 | 0 | 0.592394 |
| 3311 | −3.83177 | 0 | 0.058112 | 0 | 0.086814 | 0 | −0.06405 | 0 | 0.760982 |
| 3312 | −3.62749 | 0 | 0.054029 | 0 | 0.071049 | 0 | −0.06742 | 0 | 0.659976 |
| 3313 | −3.66207 | 0 | 0.0544 | 0 | 0.070512 | 0 | −0.06698 | 0 | 0.657244 |
| 3314 | −3.65325 | 0 | 0.054292 | 0 | 0.063402 | 0 | −0.07245 | 0 | 0.653927 |
| 3315 | −3.50212 | 0 | 0.051789 | 0 | 0.118095 | 0 | 0 | −0.14957 | 0.585101 |
| 3316 | −3.87561 | 0 | 0.059731 | 0 | 0.125285 | 0 | 0 | −0.175 | 0.782592 |
| 3317 | −3.66306 | 0 | 0.055391 | 0 | 0.102618 | 0 | 0 | −0.164 | 0.66807 |
| 3318 | −3.68248 | 0 | 0.055619 | 0 | 0.10969 | 0 | 0 | −0.17207 | 0.676659 |
| 3319 | −3.65134 | 0 | 0.055485 | 0 | 0.106284 | 0 | 0 | −0.19789 | 0.701174 |
| 3320 | −3.37179 | 0 | 0.049822 | 0 | 0.064664 | 0 | 0 | 0 | 0.648623 |
| 3321 | −3.45488 | 0 | 0.051997 | 0 | 0.083668 | 0 | 0 | 0 | 0.756713 |
| 3322 | −3.77474 | 0 | 0.057767 | 0 | 0.060634 | 0 | 0 | 0 | 0.789869 |
| 3323 | −3.79411 | 0 | 0.057824 | 0 | 0.061127 | 0 | 0 | 0 | 0.783929 |
| 3324 | −3.78109 | 0 | 0.057679 | 0 | 0.052363 | 0 | 0 | 0 | 0.780732 |
| 3325 | −3.34884 | 0 | 0.049347 | 0 | 0.062952 | 0 | 0 | 0 | 0.654524 |
| 3326 | −3.33264 | 0 | 0.049097 | 0 | 0.053557 | 0 | 0 | 0 | 0.646059 |
| 3327 | −3.58037 | 0 | 0.053751 | 0 | 0.047824 | 0 | 0 | 0 | 0.702203 |
| 3328 | −3.56742 | 0 | 0.053643 | 0 | 0.035953 | 0 | 0 | 0 | 0.702579 |
| 3329 | −3.61632 | 0 | 0.05409 | 0 | 0.045453 | 0 | 0 | 0 | 0.685579 |
| 3330 | −3.4647 | 0 | 0.050717 | 0 | 0 | −0.06345 | −0.05538 | 0 | 0.597127 |
| 3331 | −3.84189 | 0 | 0.058397 | 0 | 0 | −0.06493 | −0.05448 | 0 | 0.764665 |
| 3332 | −3.62921 | 0 | 0.054249 | 0 | 0 | −0.07132 | −0.05867 | 0 | 0.670198 |
| 3333 | −3.65016 | 0 | 0.054573 | 0 | 0 | −0.10008 | −0.05846 | 0 | 0.680208 |
| 3334 | −3.64977 | 0 | 0.054473 | 0 | 0 | −0.07444 | −0.06503 | 0 | 0.667571 |
| 3335 | −3.50689 | 0 | 0.051822 | 0 | 0 | −0.03651 | 0 | −0.12952 | 0.588213 |
| 3336 | −3.88426 | 0 | 0.059776 | 0 | 0 | −0.03393 | 0 | −0.15507 | 0.781546 |
| 3337 | −3.66191 | 0 | 0.055388 | 0 | 0 | −0.04214 | 0 | −0.14602 | 0.676466 |
| 3338 | −3.67126 | 0 | 0.055552 | 0 | 0 | −0.07078 | 0 | −0.14826 | 0.690925 |
| 3339 | −3.65015 | 0 | 0.055462 | 0 | 0 | −0.03869 | 0 | −0.17817 | 0.707424 |
| 3340 | −3.37417 | 0 | 0.050034 | 0 | 0 | −0.07565 | 0 | 0 | 0.65786 |
| 3341 | −3.47041 | 0 | 0.052373 | 0 | 0 | −0.07798 | 0 | 0 | 0.760562 |
| 3342 | −3.78218 | 0 | 0.058052 | 0 | 0 | −0.08099 | 0 | 0 | 0.797805 |
| 3343 | −3.79307 | 0 | 0.058138 | 0 | 0 | −0.11111 | 0 | 0 | 0.802634 |
| 3344 | −3.78521 | 0 | 0.057955 | 0 | 0 | −0.08316 | 0 | 0 | 0.791215 |
| 3345 | −3.33133 | 0 | 0.049423 | 0 | 0 | −0.10851 | 0 | 0 | 0.676964 |
| 3346 | −3.32678 | 0 | 0.049234 | 0 | 0 | −0.08215 | 0 | 0 | 0.658904 |
| 3347 | −3.56821 | 0 | 0.053936 | 0 | 0 | −0.11133 | 0 | 0 | 0.726083 |
| 3348 | −3.5623 | 0 | 0.053835 | 0 | 0 | −0.08975 | 0 | 0 | 0.718078 |
| 3349 | −3.60268 | 0 | 0.054256 | 0 | 0 | −0.10637 | 0 | 0 | 0.710351 |
| 3350 | −3.51942 | 0 | 0.051804 | 0 | 0 | 0 | −0.05031 | −0.13218 | 0.58219 |
| 3351 | −3.89374 | 0 | 0.059731 | 0 | 0 | 0 | −0.04692 | −0.15675 | 0.776743 |
| 3352 | −3.66812 | 0 | 0.055253 | 0 | 0 | 0 | −0.05156 | −0.14854 | 0.670386 |
| 3353 | −3.69175 | 0 | 0.055524 | 0 | 0 | 0 | −0.05237 | −0.15424 | 0.675048 |
| 3354 | −3.65915 | 0 | 0.055372 | 0 | 0 | 0 | −0.05764 | −0.18058 | 0.702797 |
| 3355 | −3.38178 | 0 | 0.049813 | 0 | 0 | 0 | −0.05329 | 0 | 0.647149 |
| 3356 | −3.47778 | 0 | 0.052192 | 0 | 0 | 0 | −0.04109 | 0 | 0.752006 |
| 3357 | −3.78381 | 0 | 0.05774 | 0 | 0 | 0 | −0.05203 | 0 | 0.788038 |
| 3358 | −3.80591 | 0 | 0.057848 | 0 | 0 | 0 | −0.05203 | 0 | 0.780988 |
| 3359 | −3.792 | 0 | 0.057685 | 0 | 0 | 0 | −0.05863 | 0 | 0.779845 |
| 3360 | −3.36169 | 0 | 0.049395 | 0 | 0 | 0 | −0.05224 | 0 | 0.652192 |
| 3361 | −3.34082 | 0 | 0.049075 | 0 | 0 | 0 | −0.05797 | 0 | 0.646421 |
| 3362 | −3.58536 | 0 | 0.053676 | 0 | 0 | 0 | −0.05694 | 0 | 0.701546 |
| 3363 | −3.5685 | 0 | 0.05351 | 0 | 0 | 0 | −0.06269 | 0 | 0.704165 |
| 3364 | −3.6239 | 0 | 0.054053 | 0 | 0 | 0 | −0.06282 | 0 | 0.685881 |
| 3365 | −3.4281 | 0 | 0.051124 | 0 | 0 | 0 | 0 | −0.14012 | 0.64599 |
| 3366 | −3.53295 | 0 | 0.053704 | 0 | 0 | 0 | 0 | −0.1481 | 0.760341 |
| 3367 | −3.83881 | 0 | 0.059492 | 0 | 0 | 0 | 0 | −0.16931 | 0.813565 |
| 3368 | −3.85017 | 0 | 0.059467 | 0 | 0 | 0 | 0 | −0.17336 | 0.811664 |
| 3369 | −3.83084 | 0 | 0.059529 | 0 | 0 | 0 | 0 | −0.20198 | 0.835621 |
| 3370 | −3.39161 | 0 | 0.050511 | 0 | 0 | 0 | 0 | −0.14576 | 0.658555 |
| 3371 | −3.34143 | 0 | 0.050024 | 0 | 0 | 0 | 0 | −0.17514 | 0.678488 |
| 3372 | −3.61188 | 0 | 0.054938 | 0 | 0 | 0 | 0 | −0.1638 | 0.723317 |
| 3373 | −3.57822 | 0 | 0.054852 | 0 | 0 | 0 | 0 | −0.19362 | 0.754282 |
| 3374 | −3.62546 | 0 | 0.055189 | 0 | 0 | 0 | 0 | −0.18572 | 0.73262 |
| 3375 | −3.44675 | 0 | 0.05212 | 0 | 0 | 0 | 0 | 0 | 0.777107 |
| 3376 | −3.42516 | 0 | 0.051615 | 0 | 0 | 0 | 0 | 0 | 0.774579 |
| 3377 | −3.39206 | 0 | 0.051128 | 0 | 0 | 0 | 0 | 0 | 0.770121 |
| 3378 | −3.75429 | 0 | 0.057515 | 0 | 0 | 0 | 0 | 0 | 0.806474 |
| 3379 | −3.73894 | 0 | 0.057369 | 0 | 0 | 0 | 0 | 0 | 0.804826 |
| 3380 | −3.76979 | 0 | 0.057553 | 0 | 0 | 0 | 0 | 0 | 0.794927 |
| 3381 | −3.30359 | 0 | 0.049 | 0 | 0 | 0 | 0 | 0 | 0.689703 |
| 3382 | −3.27619 | 0 | 0.048645 | 0 | 0 | 0 | 0 | 0 | 0.688252 |
| 3383 | −3.28815 | 0 | 0.048581 | 0 | 0 | 0 | 0 | 0 | 0.677874 |
| 3384 | −3.54906 | 0 | 0.053474 | 0 | 0 | 0 | 0 | 0 | 0.724595 |
| 3385 | −0.21751 | 0 | 0 | 0.14094 | 0.07353 | −0.05753 | 0 | 0 | 0.854108 |
| 3386 | −0.23855 | 0 | 0 | 0.101375 | 0.070174 | −0.05034 | 0 | 0 | 0.831394 |
| 3387 | −0.21553 | 0 | 0 | 0.105714 | 0.053604 | −0.05979 | 0 | 0 | 0.838462 |
| 3388 | −0.22419 | 0 | 0 | 0.117253 | 0.058745 | −0.09738 | 0 | 0 | 0.84448 |
| 3389 | −0.23423 | 0 | 0 | 0.103465 | 0.045617 | −0.06266 | 0 | 0 | 0.825004 |
| 3390 | −0.23972 | 0 | 0 | 0.156375 | 0.068992 | 0 | −0.07779 | 0 | 0.845692 |
| 3391 | −0.25841 | 0 | 0 | 0.11785 | 0.067138 | 0 | −0.07898 | 0 | 0.824178 |
| 3392 | −0.23783 | 0 | 0 | 0.121996 | 0.049231 | 0 | −0.08442 | 0 | 0.830406 |
| 3393 | −0.2581 | 0 | 0 | 0.135735 | 0.045231 | 0 | −0.08289 | 0 | 0.821429 |
| 3394 | −0.25783 | 0 | 0 | 0.121997 | 0.040754 | 0 | −0.08964 | 0 | 0.817048 |
| 3395 | −0.20428 | 0 | 0 | 0.147994 | 0.093077 | 0 | 0 | −0.12025 | 0.840953 |
| 3396 | −0.2113 | 0 | 0 | 0.113382 | 0.098257 | 0 | 0 | −0.14128 | 0.835309 |
| 3397 | −0.19394 | 0 | 0 | 0.11597 | 0.076903 | 0 | 0 | −0.13744 | 0.83512 |
| 3398 | −0.20796 | 0 | 0 | 0.130251 | 0.0778 | 0 | 0 | −0.14743 | 0.836912 |
| 3399 | −0.18649 | 0 | 0 | 0.119694 | 0.078763 | 0 | 0 | −0.1766 | 0.85712 |
| 3400 | −0.20368 | 0 | 0 | 0.138323 | 0.042543 | 0 | 0 | 0 | 0.868914 |
| 3401 | −0.20417 | 0 | 0 | 0.151561 | 0.065427 | 0 | 0 | 0 | 0.894238 |
| 3402 | −0.21602 | 0 | 0 | 0.102756 | 0.039954 | 0 | 0 | 0 | 0.855926 |
| 3403 | −0.23515 | 0 | 0 | 0.117702 | 0.035656 | 0 | 0 | 0 | 0.849669 |
| 3404 | −0.23518 | 0 | 0 | 0.102265 | 0.032009 | 0 | 0 | 0 | 0.844246 |
| 3405 | −0.21374 | 0 | 0 | 0.16049 | 0.035965 | 0 | 0 | 0 | 0.877297 |
| 3406 | −0.21234 | 0 | 0 | 0.143831 | 0.027964 | 0 | 0 | 0 | 0.870068 |
| 3407 | −0.21648 | 0 | 0 | 0.120509 | 0.020836 | 0 | 0 | 0 | 0.854267 |
| 3408 | −0.21172 | 0 | 0 | 0.105981 | 0.011363 | 0 | 0 | 0 | 0.853591 |
| 3409 | −0.23696 | 0 | 0 | 0.11789 | 0.019586 | 0 | 0 | 0 | 0.836556 |
| 3410 | −0.23405 | 0 | 0 | 0.16176 | 0 | −0.03893 | −0.07091 | 0 | 0.850069 |
| 3411 | −0.25423 | 0 | 0 | 0.123715 | 0 | −0.0318 | −0.07242 | 0 | 0.827714 |
| 3412 | −0.22923 | 0 | 0 | 0.125311 | 0 | −0.04555 | −0.07915 | 0 | 0.838736 |
| 3413 | −0.23882 | 0 | 0 | 0.1384 | 0 | −0.08191 | −0.07729 | 0 | 0.842579 |
| 3414 | −0.24758 | 0 | 0 | 0.124736 | 0 | −0.05029 | −0.08535 | 0 | 0.827577 |
| 3415 | −0.20619 | 0 | 0 | 0.156451 | 0 | −0.01892 | 0 | −0.10516 | 0.844476 |
| 3416 | −0.2154 | 0 | 0 | 0.123312 | 0 | −0.00984 | 0 | −0.12707 | 0.836802 |
| 3417 | −0.19269 | 0 | 0 | 0.122462 | 0 | −0.0233 | 0 | −0.12457 | 0.842392 |
| 3418 | −0.19875 | 0 | 0 | 0.13633 | 0 | −0.05899 | 0 | −0.12936 | 0.853008 |
| 3419 | −0.18574 | 0 | 0 | 0.12687 | 0 | −0.02093 | 0 | −0.16267 | 0.86382 |
| 3420 | −0.19475 | 0 | 0 | 0.141524 | 0 | −0.05474 | 0 | 0 | 0.876705 |
| 3421 | −0.19855 | 0 | 0 | 0.157254 | 0 | −0.05104 | 0 | 0 | 0.897716 |
| 3422 | −0.20787 | 0 | 0 | 0.10542 | 0 | −0.05238 | 0 | 0 | 0.863429 |
| 3423 | −0.2178 | 0 | 0 | 0.119588 | 0 | −0.08608 | 0 | 0 | 0.867664 |
| 3424 | −0.22602 | 0 | 0 | 0.104213 | 0 | −0.05481 | 0 | 0 | 0.852806 |
| 3425 | −0.19208 | 0 | 0 | 0.163376 | 0 | −0.10004 | 0 | 0 | 0.899081 |
| 3426 | −0.20042 | 0 | 0 | 0.145571 | 0 | −0.06545 | 0 | 0 | 0.880473 |
| 3427 | −0.19504 | 0 | 0 | 0.120559 | 0 | −0.09522 | 0 | 0 | 0.875882 |
| 3428 | −0.19813 | 0 | 0 | 0.105163 | 0 | −0.06966 | 0 | 0 | 0.86552 |
| 3429 | −0.21659 | 0 | 0 | 0.118139 | 0 | −0.08991 | 0 | 0 | 0.858073 |
| 3430 | −0.22057 | 0 | 0 | 0.169182 | 0 | 0 | −0.06818 | −0.10534 | 0.839948 |
| 3431 | −0.2279 | 0 | 0 | 0.136172 | 0 | 0 | −0.06802 | −0.12556 | 0.832694 |
| 3432 | −0.20729 | 0 | 0 | 0.136248 | 0 | 0 | −0.07541 | −0.12456 | 0.838609 |
| 3433 | −0.22144 | 0 | 0 | 0.151822 | 0 | 0 | −0.07438 | −0.13342 | 0.83894 |
| 3434 | −0.2003 | 0 | 0 | 0.142574 | 0 | 0 | −0.08153 | −0.1629 | 0.862138 |
| 3435 | −0.21554 | 0 | 0 | 0.154966 | 0 | 0 | −0.07156 | 0 | 0.86804 |
| 3436 | −0.21668 | 0 | 0 | 0.168756 | 0 | 0 | −0.05468 | 0 | 0.889047 |
| 3437 | −0.22725 | 0 | 0 | 0.120484 | 0 | 0 | −0.07434 | 0 | 0.855612 |
| 3438 | −0.24621 | 0 | 0 | 0.135484 | 0 | 0 | −0.07332 | 0 | 0.848563 |
| 3439 | −0.247 | 0 | 0 | 0.120841 | 0 | 0 | −0.07887 | 0 | 0.844222 |
| 3440 | −0.22516 | 0 | 0 | 0.176943 | 0 | 0 | −0.06871 | 0 | 0.875289 |
| 3441 | −0.22418 | 0 | 0 | 0.161231 | 0 | 0 | −0.07711 | 0 | 0.870289 |
| 3442 | −0.22735 | 0 | 0 | 0.136552 | 0 | 0 | −0.07982 | 0 | 0.854179 |
| 3443 | −0.22285 | 0 | 0 | 0.122209 | 0 | 0 | −0.08619 | 0 | 0.854935 |
| 3444 | −0.24873 | 0 | 0 | 0.135491 | 0 | 0 | −0.08384 | 0 | 0.836975 |
| 3445 | −0.18054 | 0 | 0 | 0.150561 | 0 | 0 | 0 | −0.11593 | 0.868415 |
| 3446 | −0.18026 | 0 | 0 | 0.169324 | 0 | 0 | 0 | −0.11942 | 0.895751 |
| 3447 | −0.18046 | 0 | 0 | 0.120308 | 0 | 0 | 0 | −0.13854 | 0.871633 |
| 3448 | −0.19458 | 0 | 0 | 0.137084 | 0 | 0 | 0 | −0.1452 | 0.872629 |
| 3449 | −0.17815 | 0 | 0 | 0.125613 | 0 | 0 | 0 | −0.17177 | 0.888468 |
| 3450 | −0.18417 | 0 | 0 | 0.174214 | 0 | 0 | 0 | −0.12686 | 0.885143 |
| 3451 | −0.16159 | 0 | 0 | 0.163037 | 0 | 0 | 0 | −0.16357 | 0.903941 |
| 3452 | −0.1769 | 0 | 0 | 0.135583 | 0 | 0 | 0 | −0.14395 | 0.87406 |
| 3453 | −0.15116 | 0 | 0 | 0.124628 | 0 | 0 | 0 | −0.17542 | 0.899091 |
| 3454 | −0.17993 | 0 | 0 | 0.138041 | 0 | 0 | 0 | −0.16943 | 0.881364 |
| 3455 | −0.18911 | 0 | 0 | 0.149948 | 0 | 0 | 0 | 0 | 0.903069 |
| 3456 | −0.19782 | 0 | 0 | 0.172831 | 0 | 0 | 0 | 0 | 0.910283 |
| 3457 | −0.19776 | 0 | 0 | 0.154343 | 0 | 0 | 0 | 0 | 0.903998 |
| 3458 | −0.21021 | 0 | 0 | 0.117084 | 0 | 0 | 0 | 0 | 0.872134 |
| 3459 | −0.2067 | 0 | 0 | 0.101461 | 0 | 0 | 0 | 0 | 0.870646 |
| 3460 | −0.22938 | 0 | 0 | 0.115884 | 0 | 0 | 0 | 0 | 0.858666 |
| 3461 | −0.19574 | 0 | 0 | 0.156153 | 0 | 0 | 0 | 0 | 0.890588 |
| 3462 | −0.19148 | 0 | 0 | 0.13895 | 0 | 0 | 0 | 0 | 0.887527 |
| 3463 | −0.20602 | 0 | 0 | 0.15898 | 0 | 0 | 0 | 0 | 0.88714 |
| 3464 | −0.20848 | 0 | 0 | 0.115954 | 0 | 0 | 0 | 0 | 0.864702 |
| 3465 | −0.24458 | 0 | 0 | 0 | 0.098817 | −0.06446 | −0.06122 | 0 | 0.822345 |
| 3466 | −0.25552 | 0 | 0 | 0 | 0.093098 | −0.05687 | −0.06555 | 0 | 0.816191 |
| 3467 | −0.23428 | 0 | 0 | 0 | 0.076727 | −0.06604 | −0.07164 | 0 | 0.823058 |
| 3468 | −0.24522 | 0 | 0 | 0 | 0.083526 | −0.10332 | −0.06793 | 0 | 0.822348 |
| 3469 | −0.25259 | 0 | 0 | 0 | 0.070071 | −0.06913 | −0.07632 | 0 | 0.811123 |
| 3470 | −0.21557 | 0 | 0 | 0 | 0.117708 | −0.04598 | 0 | −0.11332 | 0.816843 |
| 3471 | −0.21483 | 0 | 0 | 0 | 0.118942 | −0.03796 | 0 | −0.13461 | 0.824996 |
| 3472 | −0.19677 | 0 | 0 | 0 | 0.098448 | −0.04656 | 0 | −0.13044 | 0.825412 |
| 3473 | −0.20433 | 0 | 0 | 0 | 0.109376 | −0.08418 | 0 | −0.13711 | 0.832137 |
| 3474 | −0.19027 | 0 | 0 | 0 | 0.10115 | −0.04551 | 0 | −0.16821 | 0.845557 |
| 3475 | −0.20486 | 0 | 0 | 0 | 0.074718 | −0.07414 | 0 | 0 | 0.854304 |
| 3476 | −0.20528 | 0 | 0 | 0 | 0.101781 | −0.0789 | 0 | 0 | 0.876815 |
| 3477 | −0.21022 | 0 | 0 | 0 | 0.068593 | −0.07104 | 0 | 0 | 0.853307 |
| 3478 | −0.22194 | 0 | 0 | 0 | 0.07542 | −0.10624 | 0 | 0 | 0.852904 |
| 3479 | −0.22869 | 0 | 0 | 0 | 0.061926 | −0.07226 | 0 | 0 | 0.842069 |
| 3480 | −0.20635 | 0 | 0 | 0 | 0.082016 | −0.12004 | 0 | 0 | 0.867486 |
| 3481 | −0.2126 | 0 | 0 | 0 | 0.063806 | −0.08233 | 0 | 0 | 0.85482 |
| 3482 | −0.20201 | 0 | 0 | 0 | 0.05993 | −0.11104 | 0 | 0 | 0.859018 |
| 3483 | −0.20337 | 0 | 0 | 0 | 0.042432 | −0.08187 | 0 | 0 | 0.853476 |
| 3484 | −0.22315 | 0 | 0 | 0 | 0.058189 | −0.10507 | 0 | 0 | 0.841205 |
| 3485 | −0.23368 | 0 | 0 | 0 | 0.116607 | 0 | −0.05918 | −0.11663 | 0.808078 |
| 3486 | −0.23077 | 0 | 0 | 0 | 0.119706 | 0 | −0.0622 | −0.13607 | 0.817885 |
| 3487 | −0.21505 | 0 | 0 | 0 | 0.098548 | 0 | −0.0685 | −0.13282 | 0.817896 |
| 3488 | −0.23267 | 0 | 0 | 0 | 0.101036 | 0 | −0.06464 | −0.14234 | 0.810976 |
| 3489 | −0.20872 | 0 | 0 | 0 | 0.101545 | 0 | −0.07306 | −0.17074 | 0.839217 |
| 3490 | −0.22857 | 0 | 0 | 0 | 0.066255 | 0 | −0.06027 | 0 | 0.842584 |
| 3491 | −0.2269 | 0 | 0 | 0 | 0.089248 | 0 | −0.04424 | 0 | 0.864908 |
| 3492 | −0.2324 | 0 | 0 | 0 | 0.061921 | 0 | −0.06569 | 0 | 0.842827 |
| 3493 | −0.25398 | 0 | 0 | 0 | 0.059745 | 0 | −0.06215 | 0 | 0.829294 |
| 3494 | −0.25199 | 0 | 0 | 0 | 0.054823 | 0 | −0.0689 | 0 | 0.830604 |
| 3495 | −0.24302 | 0 | 0 | 0 | 0.062208 | 0 | −0.0539 | 0 | 0.838926 |
| 3496 | −0.23854 | 0 | 0 | 0 | 0.053979 | 0 | −0.06295 | 0 | 0.841254 |
| 3497 | −0.23743 | 0 | 0 | 0 | 0.044547 | 0 | −0.06754 | 0 | 0.833597 |
| 3498 | −0.22991 | 0 | 0 | 0 | 0.034237 | 0 | −0.07462 | 0 | 0.840544 |
| 3499 | −0.2581 | 0 | 0 | 0 | 0.044657 | 0 | −0.07124 | 0 | 0.816155 |
| 3500 | −0.19331 | 0 | 0 | 0 | 0.091373 | 0 | 0 | −0.12318 | 0.841685 |
| 3501 | −0.18988 | 0 | 0 | 0 | 0.119577 | 0 | 0 | −0.12807 | 0.870754 |
| 3502 | −0.18499 | 0 | 0 | 0 | 0.092659 | 0 | 0 | −0.14487 | 0.856863 |
| 3503 | −0.20277 | 0 | 0 | 0 | 0.096118 | 0 | 0 | −0.15234 | 0.850948 |
| 3504 | −0.18316 | 0 | 0 | 0 | 0.096304 | 0 | 0 | −0.17846 | 0.872132 |
| 3505 | −0.20337 | 0 | 0 | 0 | 0.092526 | 0 | 0 | −0.13419 | 0.846973 |
| 3506 | −0.17782 | 0 | 0 | 0 | 0.090948 | 0 | 0 | −0.16886 | 0.871895 |
| 3507 | −0.18838 | 0 | 0 | 0 | 0.07701 | 0 | 0 | −0.14903 | 0.850122 |
| 3508 | −0.16021 | 0 | 0 | 0 | 0.071482 | 0 | 0 | −0.17895 | 0.879727 |
| 3509 | −0.19159 | 0 | 0 | 0 | 0.083137 | 0 | 0 | −0.17469 | 0.855881 |
| 3510 | −0.19979 | 0 | 0 | 0 | 0.066119 | 0 | 0 | 0 | 0.882076 |
| 3511 | −0.21327 | 0 | 0 | 0 | 0.059762 | 0 | 0 | 0 | 0.879083 |
| 3512 | −0.20947 | 0 | 0 | 0 | 0.052125 | 0 | 0 | 0 | 0.880426 |
| 3513 | −0.2176 | 0 | 0 | 0 | 0.035759 | 0 | 0 | 0 | 0.857629 |
| 3514 | −0.21147 | 0 | 0 | 0 | 0.025153 | 0 | 0 | 0 | 0.861547 |
| 3515 | −0.23647 | 0 | 0 | 0 | 0.03533 | 0 | 0 | 0 | 0.84377 |
| 3516 | −0.21213 | 0 | 0 | 0 | 0.03986 | 0 | 0 | 0 | 0.862142 |
| 3517 | −0.20396 | 0 | 0 | 0 | 0.026607 | 0 | 0 | 0 | 0.867227 |
| 3518 | −0.22321 | 0 | 0 | 0 | 0.035066 | 0 | 0 | 0 | 0.856549 |
| 3519 | −0.21669 | 0 | 0 | 0 | 0.016699 | 0 | 0 | 0 | 0.850925 |
| 3520 | −5.2412 | 1.473481 | 0.065413 | 0.198986 | 0 | 0 | 0 | 0 | 0.550788 |
| 3521 | −5.55002 | 1.367612 | 0.072112 | 0.194921 | 0 | 0 | 0 | 0 | 0.632455 |
| 3522 | −5.42073 | 1.432529 | 0.06884 | 0.17759 | 0 | 0 | 0 | 0 | 0.548819 |
| 3523 | −5.41627 | 1.409411 | 0.068806 | 0.190166 | 0 | 0 | 0 | 0 | 0.540958 |
| 3524 | −5.41737 | 1.431141 | 0.068739 | 0.182374 | 0 | 0 | 0 | 0 | 0.548933 |
| 3525 | −5.12135 | 1.492746 | 0.062983 | 0 | −0.00317 | 0 | 0 | 0 | 0.518742 |
| 3526 | −5.40469 | 1.388371 | 0.069315 | 0 | 0.002199 | 0 | 0 | 0 | 0.607773 |
| 3527 | −5.29109 | 1.452665 | 0.066374 | 0 | −0.01643 | 0 | 0 | 0 | 0.534909 |
| 3528 | −5.29014 | 1.434425 | 0.066277 | 0 | −0.01555 | 0 | 0 | 0 | 0.516438 |
| 3529 | −5.2836 | 1.453538 | 0.066168 | 0 | −0.02386 | 0 | 0 | 0 | 0.532526 |
| 3530 | −5.16495 | 1.519201 | 0.063733 | 0 | 0 | −0.13106 | 0 | 0 | 0.522468 |
| 3531 | −5.45187 | 1.414207 | 0.070075 | 0 | 0 | −0.12421 | 0 | 0 | 0.60748 |
| 3532 | −5.33124 | 1.474865 | 0.067114 | 0 | 0 | −0.13237 | 0 | 0 | 0.538944 |
| 3533 | −5.31585 | 1.457787 | 0.066955 | 0 | 0 | −0.15313 | 0 | 0 | 0.536699 |
| 3534 | −5.31749 | 1.474362 | 0.066843 | 0 | 0 | −0.13451 | 0 | 0 | 0.538117 |
| 3535 | −5.1495 | 1.493953 | 0.06324 | 0 | 0 | 0 | −0.06131 | 0 | 0.511536 |
| 3536 | −5.42941 | 1.391299 | 0.069507 | 0 | 0 | 0 | −0.0619 | 0 | 0.60118 |
| 3537 | −5.30839 | 1.451027 | 0.066482 | 0 | 0 | 0 | −0.06475 | 0 | 0.528274 |
| 3538 | −5.31439 | 1.4332 | 0.066485 | 0 | 0 | 0 | −0.06559 | 0 | 0.508764 |
| 3539 | −5.30261 | 1.449981 | 0.066316 | 0 | 0 | 0 | −0.06994 | 0 | 0.526066 |
| 3540 | −5.22705 | 1.507256 | 0.064971 | 0 | 0 | 0 | 0 | −0.14654 | 0.508895 |
| 3541 | −5.51574 | 1.406097 | 0.071664 | 0 | 0 | 0 | 0 | −0.17086 | 0.629232 |
| 3542 | −5.37316 | 1.464879 | 0.06822 | 0 | 0 | 0 | 0 | −0.16246 | 0.545834 |
| 3543 | −5.36599 | 1.445247 | 0.068104 | 0 | 0 | 0 | 0 | −0.16591 | 0.533903 |
| 3544 | −5.32859 | 1.457213 | 0.067842 | 0 | 0 | 0 | 0 | −0.18422 | 0.576442 |
| 3545 | −5.06378 | 1.473423 | 0.062638 | 0 | 0 | 0 | 0 | 0 | 0.55957 |
| 3546 | −5.10754 | 1.401562 | 0.064624 | 0 | 0 | 0 | 0 | 0 | 0.651116 |
| 3547 | −5.35774 | 1.367922 | 0.069091 | 0 | 0 | 0 | 0 | 0 | 0.636421 |
| 3548 | −5.34097 | 1.351413 | 0.068743 | 0 | 0 | 0 | 0 | 0 | 0.623483 |
| 3549 | −5.33894 | 1.371165 | 0.068661 | 0 | 0 | 0 | 0 | 0 | 0.632431 |
| 3550 | −5.0212 | 1.449601 | 0.061977 | 0 | 0 | 0 | 0 | 0 | 0.545869 |
| 3551 | −4.99745 | 1.471431 | 0.06156 | 0 | 0 | 0 | 0 | 0 | 0.561376 |
| 3552 | −5.22271 | 1.414015 | 0.065818 | 0 | 0 | 0 | 0 | 0 | 0.558755 |
| 3553 | −5.2087 | 1.427587 | 0.065658 | 0 | 0 | 0 | 0 | 0 | 0.574107 |
| 3554 | −5.2374 | 1.421732 | 0.065818 | 0 | 0 | 0 | 0 | 0 | 0.546237 |
| 3555 | −0.95278 | 1.227156 | 0 | 0.107978 | −0.00871 | 0 | 0 | 0 | 0.802141 |
| 3556 | −0.9519 | 1.093372 | 0 | 0.071588 | −0.00523 | 0 | 0 | 0 | 0.686874 |
| 3557 | −0.94867 | 1.16196 | 0 | 0.071866 | −0.02684 | 0 | 0 | 0 | 0.729719 |
| 3558 | −0.96478 | 1.146465 | 0 | 0.086078 | −0.02779 | 0 | 0 | 0 | 0.711074 |
| 3559 | −0.96934 | 1.170034 | 0 | 0.071672 | −0.03487 | 0 | 0 | 0 | 0.72036 |
| 3560 | −0.94781 | 1.241772 | 0 | 0.103473 | 0 | −0.09052 | 0 | 0 | 0.806772 |
| 3561 | −0.9495 | 1.105645 | 0 | 0.067453 | 0 | −0.07537 | 0 | 0 | 0.688237 |
| 3562 | −0.93984 | 1.170601 | 0 | 0.064995 | 0 | −0.09001 | 0 | 0 | 0.734226 |
| 3563 | −0.94642 | 1.157321 | 0 | 0.078571 | 0 | −0.11994 | 0 | 0 | 0.729617 |
| 3564 | −0.95876 | 1.177677 | 0 | 0.063622 | 0 | −0.09468 | 0 | 0 | 0.725817 |
| 3565 | −0.96065 | 1.221787 | 0 | 0.117963 | 0 | 0 | −0.06174 | 0 | 0.797185 |
| 3566 | −0.96056 | 1.089632 | 0 | 0.08321 | 0 | 0 | −0.06727 | 0 | 0.681959 |
| 3567 | −0.95442 | 1.152973 | 0 | 0.082521 | 0 | 0 | −0.07517 | 0 | 0.725942 |
| 3568 | −0.97032 | 1.136516 | 0 | 0.095465 | 0 | 0 | −0.07146 | 0 | 0.705175 |
| 3569 | −0.97366 | 1.157883 | 0 | 0.081919 | 0 | 0 | −0.07823 | 0 | 0.716208 |
| 3570 | −0.93189 | 1.22746 | 0 | 0.116278 | 0 | 0 | 0 | −0.10841 | 0.796499 |
| 3571 | −0.92069 | 1.093989 | 0 | 0.084013 | 0 | 0 | 0 | −0.12773 | 0.69508 |
| 3572 | −0.91514 | 1.158792 | 0 | 0.081014 | 0 | 0 | 0 | −0.12832 | 0.736136 |
| 3573 | −0.92492 | 1.139956 | 0 | 0.09648 | 0 | 0 | 0 | −0.1341 | 0.724279 |
| 3574 | −0.90666 | 1.156365 | 0 | 0.085113 | 0 | 0 | 0 | −0.15674 | 0.757563 |
| 3575 | −0.91955 | 1.198021 | 0 | 0.098562 | 0 | 0 | 0 | 0 | 0.812296 |
| 3576 | −0.88685 | 1.098936 | 0 | 0.118209 | 0 | 0 | 0 | 0 | 0.808503 |
| 3577 | −0.90466 | 1.063593 | 0 | 0.066686 | 0 | 0 | 0 | 0 | 0.720295 |
| 3578 | −0.91797 | 1.052874 | 0 | 0.081071 | 0 | 0 | 0 | 0 | 0.70993 |
| 3579 | −0.92495 | 1.073994 | 0 | 0.066634 | 0 | 0 | 0 | 0 | 0.713908 |
| 3580 | −0.92231 | 1.174055 | 0 | 0.119862 | 0 | 0 | 0 | 0 | 0.807602 |
| 3581 | −0.92748 | 1.203257 | 0 | 0.102601 | 0 | 0 | 0 | 0 | 0.818031 |
| 3582 | −0.92043 | 1.117087 | 0 | 0.07844 | 0 | 0 | 0 | 0 | 0.739366 |
| 3583 | −0.91826 | 1.134322 | 0 | 0.063586 | 0 | 0 | 0 | 0 | 0.751364 |
| 3584 | −0.94611 | 1.132269 | 0 | 0.076855 | 0 | 0 | 0 | 0 | 0.725947 |
| 3585 | −0.96491 | 1.252014 | 0 | 0 | 0.025702 | −0.09947 | 0 | 0 | 0.787045 |
| 3586 | −0.95701 | 1.112109 | 0 | 0 | 0.022244 | −0.0829 | 0 | 0 | 0.680361 |
| 3587 | −0.95093 | 1.181099 | 0 | 0 | 0.002339 | −0.09303 | 0 | 0 | 0.726679 |
| 3588 | −0.95971 | 1.168355 | 0 | 0 | 0.010693 | −0.12466 | 0 | 0 | 0.717339 |
| 3589 | −0.97019 | 1.189251 | 0 | 0 | −0.00425 | −0.0962 | 0 | 0 | 0.719061 |
| 3590 | −0.98135 | 1.236367 | 0 | 0 | 0.011015 | 0 | −0.04915 | 0 | 0.775016 |
| 3591 | −0.97155 | 1.100268 | 0 | 0 | 0.012096 | 0 | −0.05802 | 0 | 0.672193 |
| 3592 | −0.96857 | 1.168255 | 0 | 0 | −0.00819 | 0 | −0.06347 | 0 | 0.716889 |
| 3593 | −0.98843 | 1.154626 | 0 | 0 | −0.00823 | 0 | −0.05884 | 0 | 0.690764 |
| 3594 | −0.98828 | 1.17522 | 0 | 0 | −0.0159 | 0 | −0.06633 | 0 | 0.70824 |
| 3595 | −0.94977 | 1.234349 | 0 | 0 | 0.037035 | 0 | 0 | −0.10884 | 0.773926 |
| 3596 | −0.92656 | 1.096049 | 0 | 0 | 0.044571 | 0 | 0 | −0.12986 | 0.685654 |
| 3597 | −0.92658 | 1.166279 | 0 | 0 | 0.020025 | 0 | 0 | −0.12724 | 0.725538 |
| 3598 | −0.94041 | 1.148909 | 0 | 0 | 0.024892 | 0 | 0 | −0.13295 | 0.70723 |
| 3599 | −0.91874 | 1.164121 | 0 | 0 | 0.021267 | 0 | 0 | −0.15495 | 0.745818 |
| 3600 | −0.94121 | 1.216208 | 0 | 0 | −0.01354 | 0 | 0 | 0 | 0.796475 |
| 3601 | −0.90749 | 1.113956 | 0 | 0 | 0.014416 | 0 | 0 | 0 | 0.787544 |
| 3602 | −0.91782 | 1.078053 | 0 | 0 | −0.01217 | 0 | 0 | 0 | 0.713763 |
| 3603 | −0.93508 | 1.070099 | 0 | 0 | −0.01126 | 0 | 0 | 0 | 0.6983 |
| 3604 | −0.93881 | 1.090063 | 0 | 0 | −0.01904 | 0 | 0 | 0 | 0.707833 |
| 3605 | −0.95045 | 1.196574 | 0 | 0 | −0.01522 | 0 | 0 | 0 | 0.783034 |
| 3606 | −0.95148 | 1.225285 | 0 | 0 | −0.02599 | 0 | 0 | 0 | 0.802164 |
| 3607 | −0.93912 | 1.138255 | 0 | 0 | −0.03089 | 0 | 0 | 0 | 0.731097 |
| 3608 | −0.93374 | 1.155923 | 0 | 0 | −0.04365 | 0 | 0 | 0 | 0.750561 |
| 3609 | −0.96453 | 1.153269 | 0 | 0 | −0.03243 | 0 | 0 | 0 | 0.718062 |
| 3610 | −0.97861 | 1.255985 | 0 | 0 | 0 | −0.09255 | −0.04567 | 0 | 0.780314 |
| 3611 | −0.97144 | 1.116872 | 0 | 0 | 0 | −0.07636 | −0.05504 | 0 | 0.673734 |
| 3612 | −0.96136 | 1.180757 | 0 | 0 | 0 | −0.09036 | −0.06212 | 0 | 0.722846 |
| 3613 | −0.97142 | 1.169672 | 0 | 0 | 0 | −0.12061 | −0.05693 | 0 | 0.711493 |
| 3614 | −0.97918 | 1.186603 | 0 | 0 | 0 | −0.09501 | −0.06579 | 0 | 0.715713 |
| 3615 | −0.95544 | 1.255401 | 0 | 0 | 0 | −0.0716 | 0 | −0.09442 | 0.777141 |
| 3616 | −0.93641 | 1.115894 | 0 | 0 | 0 | −0.05247 | 0 | −0.11742 | 0.68416 |
| 3617 | −0.92759 | 1.180922 | 0 | 0 | 0 | −0.0662 | 0 | −0.11648 | 0.729765 |
| 3618 | −0.93563 | 1.167652 | 0 | 0 | 0 | −0.09605 | 0 | −0.11779 | 0.722031 |
| 3619 | −0.92096 | 1.179517 | 0 | 0 | 0 | −0.06473 | 0 | −0.14366 | 0.749209 |
| 3620 | −0.93232 | 1.230742 | 0 | 0 | 0 | −0.10418 | 0 | 0 | 0.804166 |
| 3621 | −0.90597 | 1.134616 | 0 | 0 | 0 | −0.09738 | 0 | 0 | 0.792035 |
| 3622 | −0.91043 | 1.088847 | 0 | 0 | 0 | −0.09078 | 0 | 0 | 0.718925 |
| 3623 | −0.91957 | 1.082695 | 0 | 0 | 0 | −0.11854 | 0 | 0 | 0.715238 |
| 3624 | −0.93003 | 1.099829 | 0 | 0 | 0 | −0.09398 | 0 | 0 | 0.713743 |
| 3625 | −0.93063 | 1.215818 | 0 | 0 | 0 | −0.14431 | 0 | 0 | 0.807074 |
| 3626 | −0.93938 | 1.238664 | 0 | 0 | 0 | −0.11491 | 0 | 0 | 0.812091 |
| 3627 | −0.91718 | 1.147652 | 0 | 0 | 0 | −0.13037 | 0 | 0 | 0.75089 |
| 3628 | −0.91793 | 1.161227 | 0 | 0 | 0 | −0.10999 | 0 | 0 | 0.758354 |
| 3629 | −0.94366 | 1.162759 | 0 | 0 | 0 | −0.1269 | 0 | 0 | 0.737661 |
| 3630 | −0.96594 | 1.242242 | 0 | 0 | 0 | 0 | −0.04399 | −0.10199 | 0.76795 |
| 3631 | −0.94626 | 1.107597 | 0 | 0 | 0 | 0 | −0.05056 | −0.12127 | 0.678197 |
| 3632 | −0.94015 | 1.1711 | 0 | 0 | 0 | 0 | −0.05877 | −0.12214 | 0.722739 |
| 3633 | −0.95477 | 1.154667 | 0 | 0 | 0 | 0 | −0.05381 | −0.12702 | 0.702285 |
| 3634 | −0.93259 | 1.168798 | 0 | 0 | 0 | 0 | −0.0617 | −0.14943 | 0.743322 |
| 3635 | −0.94825 | 1.211952 | 0 | 0 | 0 | 0 | −0.05072 | 0 | 0.791263 |
| 3636 | −0.91706 | 1.117509 | 0 | 0 | 0 | 0 | −0.03388 | 0 | 0.782423 |
| 3637 | −0.92544 | 1.074725 | 0 | 0 | 0 | 0 | −0.0591 | 0 | 0.70927 |
| 3638 | −0.94241 | 1.066713 | 0 | 0 | 0 | 0 | −0.05527 | 0 | 0.692643 |
| 3639 | −0.94534 | 1.084295 | 0 | 0 | 0 | 0 | −0.06206 | 0 | 0.702901 |
| 3640 | −0.95606 | 1.191584 | 0 | 0 | 0 | 0 | −0.04344 | 0 | 0.777056 |
| 3641 | −0.95658 | 1.217487 | 0 | 0 | 0 | 0 | −0.05375 | 0 | 0.796659 |
| 3642 | −0.94466 | 1.129778 | 0 | 0 | 0 | 0 | −0.0623 | 0 | 0.724718 |
| 3643 | −0.93813 | 1.144018 | 0 | 0 | 0 | 0 | −0.07025 | 0 | 0.744003 |
| 3644 | −0.96977 | 1.143741 | 0 | 0 | 0 | 0 | −0.06431 | 0 | 0.711356 |
| 3645 | −0.91822 | 1.217087 | 0 | 0 | 0 | 0 | 0 | −0.11321 | 0.794335 |
| 3646 | −0.88893 | 1.120935 | 0 | 0 | 0 | 0 | 0 | −0.11252 | 0.786191 |
| 3647 | −0.88352 | 1.077972 | 0 | 0 | 0 | 0 | 0 | −0.13261 | 0.725991 |
| 3648 | −0.89631 | 1.067902 | 0 | 0 | 0 | 0 | 0 | −0.13634 | 0.715634 |
| 3649 | −0.88013 | 1.080226 | 0 | 0 | 0 | 0 | 0 | −0.15679 | 0.745206 |
| 3650 | −0.92155 | 1.195036 | 0 | 0 | 0 | 0 | 0 | −0.12063 | 0.787161 |
| 3651 | −0.90113 | 1.216543 | 0 | 0 | 0 | 0 | 0 | −0.1489 | 0.826352 |
| 3652 | −0.89862 | 1.132741 | 0 | 0 | 0 | 0 | 0 | −0.13725 | 0.745761 |
| 3653 | −0.86985 | 1.142178 | 0 | 0 | 0 | 0 | 0 | −0.16218 | 0.787074 |
| 3654 | −0.90627 | 1.141085 | 0 | 0 | 0 | 0 | 0 | −0.15477 | 0.752103 |
| 3655 | −0.88233 | 1.096318 | 0 | 0 | 0 | 0 | 0 | 0 | 0.797383 |
| 3656 | −0.88395 | 1.076864 | 0 | 0 | 0 | 0 | 0 | 0 | 0.793965 |
| 3657 | −0.88802 | 1.100363 | 0 | 0 | 0 | 0 | 0 | 0 | 0.803693 |
| 3658 | −0.89443 | 1.044284 | 0 | 0 | 0 | 0 | 0 | 0 | 0.725043 |
| 3659 | −0.89082 | 1.057049 | 0 | 0 | 0 | 0 | 0 | 0 | 0.737247 |
| 3660 | −0.91975 | 1.061131 | 0 | 0 | 0 | 0 | 0 | 0 | 0.714185 |
| 3661 | −0.91929 | 1.17266 | 0 | 0 | 0 | 0 | 0 | 0 | 0.796231 |
| 3662 | −0.91475 | 1.193246 | 0 | 0 | 0 | 0 | 0 | 0 | 0.814438 |
| 3663 | −0.93348 | 1.186708 | 0 | 0 | 0 | 0 | 0 | 0 | 0.797643 |
| 3664 | −0.91834 | 1.122129 | 0 | 0 | 0 | 0 | 0 | 0 | 0.746129 |
| 3665 | −0.23509 | 0 | 0 | 0 | 0 | −0.02363 | −0.04807 | −0.09758 | 0.811843 |
| 3666 | −0.23442 | 0 | 0 | 0 | 0 | −0.01491 | −0.05068 | −0.11807 | 0.819215 |
| 3667 | −0.21275 | 0 | 0 | 0 | 0 | −0.02752 | −0.0586 | −0.11646 | 0.826916 |
| 3668 | −0.22248 | 0 | 0 | 0 | 0 | −0.06286 | −0.05481 | −0.12059 | 0.828783 |
| 3669 | −0.20731 | 0 | 0 | 0 | 0 | −0.02588 | −0.06356 | −0.15269 | 0.847077 |
| 3670 | −0.21848 | 0 | 0 | 0 | 0 | −0.05625 | −0.05234 | 0 | 0.851847 |
| 3671 | −0.22005 | 0 | 0 | 0 | 0 | −0.05426 | −0.0335 | 0 | 0.868458 |
| 3672 | −0.22308 | 0 | 0 | 0 | 0 | −0.05407 | −0.05786 | 0 | 0.851766 |
| 3673 | −0.2352 | 0 | 0 | 0 | 0 | −0.08782 | −0.05448 | 0 | 0.849275 |
| 3674 | −0.24152 | 0 | 0 | 0 | 0 | −0.05654 | −0.06233 | 0 | 0.841174 |
| 3675 | −0.22024 | 0 | 0 | 0 | 0 | −0.10042 | −0.04555 | 0 | 0.862319 |
| 3676 | −0.22523 | 0 | 0 | 0 | 0 | −0.06702 | −0.05646 | 0 | 0.854001 |
| 3677 | −0.21422 | 0 | 0 | 0 | 0 | −0.09634 | −0.06132 | 0 | 0.858286 |
| 3678 | −0.21448 | 0 | 0 | 0 | 0 | −0.07109 | −0.07012 | 0 | 0.856036 |
| 3679 | −0.23615 | 0 | 0 | 0 | 0 | −0.09103 | −0.06549 | 0 | 0.84081 |
| 3680 | −0.19185 | 0 | 0 | 0 | 0 | −0.03584 | 0 | −0.10635 | 0.849797 |
| 3681 | −0.19432 | 0 | 0 | 0 | 0 | −0.03324 | 0 | −0.10784 | 0.870341 |
| 3682 | −0.18487 | 0 | 0 | 0 | 0 | −0.0317 | 0 | −0.12929 | 0.863484 |
| 3683 | −0.19598 | 0 | 0 | 0 | 0 | −0.065 | 0 | −0.13133 | 0.864464 |
| 3684 | −0.18415 | 0 | 0 | 0 | 0 | −0.0281 | 0 | −0.16109 | 0.877348 |
| 3685 | −0.19235 | 0 | 0 | 0 | 0 | −0.07863 | 0 | −0.11085 | 0.864937 |
| 3686 | −0.17598 | 0 | 0 | 0 | 0 | −0.03774 | 0 | −0.15048 | 0.879615 |
| 3687 | −0.1759 | 0 | 0 | 0 | 0 | −0.07345 | 0 | −0.12992 | 0.869906 |
| 3688 | −0.15487 | 0 | 0 | 0 | 0 | −0.04196 | 0 | −0.16403 | 0.891457 |
| 3689 | −0.18182 | 0 | 0 | 0 | 0 | −0.06459 | 0 | −0.15498 | 0.873272 |
| 3690 | −0.19096 | 0 | 0 | 0 | 0 | −0.06814 | 0 | 0 | 0.889299 |
| 3691 | −0.19251 | 0 | 0 | 0 | 0 | −0.11116 | 0 | 0 | 0.89853 |
| 3692 | −0.19757 | 0 | 0 | 0 | 0 | −0.07757 | 0 | 0 | 0.890583 |
| 3693 | −0.19673 | 0 | 0 | 0 | 0 | −0.1007 | 0 | 0 | 0.879235 |
| 3694 | −0.19734 | 0 | 0 | 0 | 0 | −0.07645 | 0 | 0 | 0.875047 |
| 3695 | −0.21708 | 0 | 0 | 0 | 0 | −0.09424 | 0 | 0 | 0.864571 |
| 3696 | −0.18831 | 0 | 0 | 0 | 0 | −0.10886 | 0 | 0 | 0.885848 |
| 3697 | −0.18817 | 0 | 0 | 0 | 0 | −0.08135 | 0 | 0 | 0.881434 |
| 3698 | −0.19958 | 0 | 0 | 0 | 0 | −0.10805 | 0 | 0 | 0.880628 |
| 3699 | −0.19279 | 0 | 0 | 0 | 0 | −0.10386 | 0 | 0 | 0.875305 |
| 3700 | −0.20621 | 0 | 0 | 0 | 0 | 0 | −0.04849 | −0.10856 | 0.842925 |
| 3701 | −0.20486 | 0 | 0 | 0 | 0 | 0 | −0.02952 | −0.11039 | 0.864364 |
| 3702 | −0.19807 | 0 | 0 | 0 | 0 | 0 | −0.05157 | −0.12997 | 0.857542 |
| 3703 | −0.2158 | 0 | 0 | 0 | 0 | 0 | −0.04876 | −0.13559 | 0.849774 |
| 3704 | −0.19741 | 0 | 0 | 0 | 0 | 0 | −0.05523 | −0.16158 | 0.871855 |
| 3705 | −0.21566 | 0 | 0 | 0 | 0 | 0 | −0.04279 | −0.11845 | 0.846171 |
| 3706 | −0.19091 | 0 | 0 | 0 | 0 | 0 | −0.05146 | −0.15326 | 0.873122 |
| 3707 | −0.19945 | 0 | 0 | 0 | 0 | 0 | −0.05651 | −0.1352 | 0.853181 |
| 3708 | −0.17113 | 0 | 0 | 0 | 0 | 0 | −0.06393 | −0.16609 | 0.885551 |
| 3709 | −0.2041 | 0 | 0 | 0 | 0 | 0 | −0.05978 | −0.15928 | 0.858539 |
| 3710 | −0.20911 | 0 | 0 | 0 | 0 | 0 | −0.03661 | 0 | 0.878898 |
| 3711 | −0.22061 | 0 | 0 | 0 | 0 | 0 | −0.03121 | 0 | 0.876587 |
| 3712 | −0.21798 | 0 | 0 | 0 | 0 | 0 | −0.03976 | 0 | 0.878378 |
| 3713 | −0.22654 | 0 | 0 | 0 | 0 | 0 | −0.05829 | 0 | 0.857291 |
| 3714 | −0.22099 | 0 | 0 | 0 | 0 | 0 | −0.0651 | 0 | 0.862017 |
| 3715 | −0.24628 | 0 | 0 | 0 | 0 | 0 | −0.06076 | 0 | 0.843011 |
| 3716 | −0.22102 | 0 | 0 | 0 | 0 | 0 | −0.04927 | 0 | 0.860803 |
| 3717 | −0.21345 | 0 | 0 | 0 | 0 | 0 | −0.0584 | 0 | 0.867117 |
| 3718 | −0.2327 | 0 | 0 | 0 | 0 | 0 | −0.05249 | 0 | 0.855105 |
| 3719 | −0.22661 | 0 | 0 | 0 | 0 | 0 | −0.06817 | 0 | 0.850831 |
| 3720 | −0.17556 | 0 | 0 | 0 | 0 | 0 | 0 | −0.12026 | 0.886023 |
| 3721 | −0.18368 | 0 | 0 | 0 | 0 | 0 | 0 | −0.12874 | 0.889294 |
| 3722 | −0.16463 | 0 | 0 | 0 | 0 | 0 | 0 | −0.16153 | 0.908389 |
| 3723 | −0.17668 | 0 | 0 | 0 | 0 | 0 | 0 | −0.14659 | 0.881438 |
| 3724 | −0.15252 | 0 | 0 | 0 | 0 | 0 | 0 | −0.17583 | 0.907311 |
| 3725 | −0.18259 | 0 | 0 | 0 | 0 | 0 | 0 | −0.16712 | 0.88573 |
| 3726 | −0.18281 | 0 | 0 | 0 | 0 | 0 | 0 | −0.1269 | 0.871344 |
| 3727 | −0.15401 | 0 | 0 | 0 | 0 | 0 | 0 | −0.16317 | 0.900041 |
| 3728 | −0.17712 | 0 | 0 | 0 | 0 | 0 | 0 | −0.15834 | 0.887669 |
| 3729 | −0.15958 | 0 | 0 | 0 | 0 | 0 | 0 | −0.17144 | 0.894483 |
| 3730 | −4.65598 | 0 | 0.065406 | 0.217247 | 0.122812 | −0.03126 | −0.10332 | −0.16681 | 0 |
| 3731 | −6.16581 | 1.547595 | 0.075122 | 0.169514 | 0.006414 | −0.11898 | −0.09298 | 0 | 0 |
| 3732 | −6.20901 | 1.548581 | 0.076517 | 0.171638 | 0.027524 | −0.09104 | 0 | −0.1589 | 0 |
| 3733 | −6.2111 | 1.529122 | 0.076369 | 0.190145 | 0.02563 | 0 | −0.092 | −0.16702 | 0 |
| 3734 | −6.24572 | 1.553195 | 0.07683 | 0.18762 | 0 | −0.08383 | −0.08757 | −0.15348 | 0 |
| 3735 | −6.07358 | 1.559482 | 0.074014 | 0 | 0.056764 | −0.10012 | −0.07569 | −0.14742 | 0 |
| 3736 | −1.40605 | 1.276739 | 0 | 0.051262 | 0.042153 | −0.05075 | −0.07986 | −0.11725 | 0 |
| 3737 | −0.19865 | 0 | 0 | 0 | 0 | 0 | 0 | 0 | 0.890616 |
| 3738 | −0.19241 | 0 | 0 | 0 | 0 | 0 | 0 | 0 | 0.894262 |
| 3739 | −0.20781 | 0 | 0 | 0 | 0 | 0 | 0 | 0 | 0.887647 |
| 3740 | −0.21027 | 0 | 0 | 0 | 0 | 0 | 0 | 0 | 0.868678 |
| 3741 | −0.20271 | 0 | 0 | 0 | 0 | 0 | 0 | 0 | 0.875728 |
| 3742 | −3.61463 | 0 | 0.053512 | 0.233904 | 0 | 0 | 0 | 0 | 0.631647 |
| 3743 | −4.02878 | 0 | 0.061703 | 0.23083 | 0 | 0 | 0 | 0 | 0.790296 |
| 3744 | −3.79791 | 0 | 0.05716 | 0.21142 | 0 | 0 | 0 | 0 | 0.681717 |
| 3745 | −3.83692 | 0 | 0.057711 | 0.229346 | 0 | 0 | 0 | 0 | 0.693109 |
| 3746 | −3.82614 | 0 | 0.057504 | 0.215415 | 0 | 0 | 0 | 0 | 0.679163 |
| 3747 | −3.43978 | 0 | 0.05037 | 0 | 0.07797 | 0 | 0 | 0 | 0.598006 |
| 3748 | −3.81733 | 0 | 0.05806 | 0 | 0.07739 | 0 | 0 | 0 | 0.766121 |
| 3749 | −3.61733 | 0 | 0.054054 | 0 | 0.060189 | 0 | 0 | 0 | 0.664732 |
| 3750 | −3.64574 | 0 | 0.054326 | 0 | 0.06086 | 0 | 0 | 0 | 0.663311 |
| 3751 | −3.641 | 0 | 0.054288 | 0 | 0.05258 | 0 | 0 | 0 | 0.658136 |
| 3752 | −3.44961 | 0 | 0.050635 | 0 | 0 | −0.06457 | 0 | 0 | 0.601769 |
| 3753 | −3.82941 | 0 | 0.058352 | 0 | 0 | −0.06645 | 0 | 0 | 0.768984 |
| 3754 | −3.62093 | 0 | 0.054283 | 0 | 0 | −0.07276 | 0 | 0 | 0.673968 |
| 3755 | −3.63605 | 0 | 0.054513 | 0 | 0 | −0.10145 | 0 | 0 | 0.685235 |
| 3756 | −3.63981 | 0 | 0.054486 | 0 | 0 | −0.07572 | 0 | 0 | 0.670507 |
| 3757 | −3.45962 | 0 | 0.050483 | 0 | 0 | 0 | −0.05637 | 0 | 0.592986 |
| 3758 | −3.83522 | 0 | 0.058142 | 0 | 0 | 0 | −0.05575 | 0 | 0.761524 |
| 3759 | −3.62442 | 0 | 0.053993 | 0 | 0 | 0 | −0.05995 | 0 | 0.664409 |
| 3760 | −3.65719 | 0 | 0.054335 | 0 | 0 | 0 | −0.06016 | 0 | 0.661306 |
| 3761 | −3.64809 | 0 | 0.054237 | 0 | 0 | 0 | −0.06613 | 0 | 0.659666 |
| 3762 | −3.50715 | 0 | 0.051751 | 0 | 0 | 0 | 0 | −0.13427 | 0.585726 |
| 3763 | −3.88374 | 0 | 0.059708 | 0 | 0 | 0 | 0 | −0.15923 | 0.78047 |
| 3764 | −3.6623 | 0 | 0.055305 | 0 | 0 | 0 | 0 | −0.15129 | 0.673474 |
| 3765 | −3.68032 | 0 | 0.055488 | 0 | 0 | 0 | 0 | −0.15676 | 0.678915 |
| 3766 | −3.65114 | 0 | 0.055397 | 0 | 0 | 0 | 0 | −0.18318 | 0.705007 |
| 3767 | −3.37089 | 0 | 0.049792 | 0 | 0 | 0 | 0 | 0 | 0.651445 |
| 3768 | −3.46449 | 0 | 0.052099 | 0 | 0 | 0 | 0 | 0 | 0.755874 |
| 3769 | −3.77657 | 0 | 0.057772 | 0 | 0 | 0 | 0 | 0 | 0.792056 |
| 3770 | −3.79392 | 0 | 0.057803 | 0 | 0 | 0 | 0 | 0 | 0.785966 |
| 3771 | −3.78139 | 0 | 0.057678 | 0 | 0 | 0 | 0 | 0 | 0.783831 |
| 3772 | −3.34611 | 0 | 0.049292 | 0 | 0 | 0 | 0 | 0 | 0.657063 |
| 3773 | −3.32857 | 0 | 0.049036 | 0 | 0 | 0 | 0 | 0 | 0.65017 |
| 3774 | −3.57643 | 0 | 0.053693 | 0 | 0 | 0 | 0 | 0 | 0.705998 |
| 3775 | −3.56352 | 0 | 0.053596 | 0 | 0 | 0 | 0 | 0 | 0.707016 |
| 3776 | −3.61228 | 0 | 0.054035 | 0 | 0 | 0 | 0 | 0 | 0.689896 |
| 3777 | −0.2265 | 0 | 0 | 0.143902 | 0.059749 | 0 | 0 | 0 | 0.849534 |
| 3778 | −0.24576 | 0 | 0 | 0.10486 | 0.057997 | 0 | 0 | 0 | 0.828338 |
| 3779 | −0.22525 | 0 | 0 | 0.109165 | 0.039427 | 0 | 0 | 0 | 0.833137 |
| 3780 | −0.24526 | 0 | 0 | 0.122341 | 0.036109 | 0 | 0 | 0 | 0.82547 |
| 3781 | −0.24485 | 0 | 0 | 0.107232 | 0.030689 | 0 | 0 | 0 | 0.81848 |
| 3782 | −0.22148 | 0 | 0 | 0.14928 | 0 | −0.04145 | 0 | 0 | 0.853697 |
| 3783 | −0.24208 | 0 | 0 | 0.110453 | 0 | −0.03472 | 0 | 0 | 0.831656 |
| 3784 | −0.2171 | 0 | 0 | 0.112023 | 0 | −0.04803 | 0 | 0 | 0.840932 |
| 3785 | −0.2263 | 0 | 0 | 0.124749 | 0 | −0.08454 | 0 | 0 | 0.846275 |
| 3786 | −0.23501 | 0 | 0 | 0.109335 | 0 | −0.05263 | 0 | 0 | 0.828224 |
| 3787 | −0.24005 | 0 | 0 | 0.16305 | 0 | 0 | −0.072 | 0 | 0.846897 |
| 3788 | −0.25878 | 0 | 0 | 0.125217 | 0 | 0 | −0.07335 | 0 | 0.825678 |
| 3789 | −0.23679 | 0 | 0 | 0.127018 | 0 | 0 | −0.08023 | 0 | 0.833986 |
| 3790 | −0.25701 | 0 | 0 | 0.140845 | 0 | 0 | −0.07931 | 0 | 0.825117 |
| 3791 | −0.25648 | 0 | 0 | 0.126563 | 0 | 0 | −0.0864 | 0 | 0.821156 |
| 3792 | −0.20861 | 0 | 0 | 0.157229 | 0 | 0 | 0 | −0.10751 | 0.842898 |
| 3793 | −0.21649 | 0 | 0 | 0.123869 | 0 | 0 | 0 | −0.12816 | 0.836267 |
| 3794 | −0.19583 | 0 | 0 | 0.123589 | 0 | 0 | 0 | −0.12725 | 0.840197 |
| 3795 | −0.2098 | 0 | 0 | 0.138713 | 0 | 0 | 0 | −0.13601 | 0.841586 |
| 3796 | −0.18853 | 0 | 0 | 0.127951 | 0 | 0 | 0 | −0.16515 | 0.861953 |
| 3797 | −0.20365 | 0 | 0 | 0.142878 | 0 | 0 | 0 | 0 | 0.871301 |
| 3798 | −0.20598 | 0 | 0 | 0.158965 | 0 | 0 | 0 | 0 | 0.894001 |
| 3799 | −0.21605 | 0 | 0 | 0.107381 | 0 | 0 | 0 | 0 | 0.85831 |
| 3800 | −0.23487 | 0 | 0 | 0.122301 | 0 | 0 | 0 | 0 | 0.852258 |
| 3801 | −0.23482 | 0 | 0 | 0.106278 | 0 | 0 | 0 | 0 | 0.8469 |
| 3802 | −0.21353 | 0 | 0 | 0.16464 | 0 | 0 | 0 | 0 | 0.879537 |
| 3803 | −0.21182 | 0 | 0 | 0.147078 | 0 | 0 | 0 | 0 | 0.872564 |
| 3804 | −0.21585 | 0 | 0 | 0.122957 | 0 | 0 | 0 | 0 | 0.856531 |
| 3805 | −0.21123 | 0 | 0 | 0.107291 | 0 | 0 | 0 | 0 | 0.855175 |
| 3806 | −0.23638 | 0 | 0 | 0.120349 | 0 | 0 | 0 | 0 | 0.838857 |
| 3807 | −0.23286 | 0 | 0 | 0 | 0.090209 | −0.06428 | 0 | 0 | 0.827505 |
| 3808 | −0.24404 | 0 | 0 | 0 | 0.084034 | −0.05703 | 0 | 0 | 0.821205 |
| 3809 | −0.22254 | 0 | 0 | 0 | 0.066613 | −0.06556 | 0 | 0 | 0.826738 |
| 3810 | −0.23329 | 0 | 0 | 0 | 0.074422 | −0.10331 | 0 | 0 | 0.827777 |
| 3811 | −0.24045 | 0 | 0 | 0 | 0.059535 | −0.06853 | 0 | 0 | 0.813981 |
| 3812 | −0.2549 | 0 | 0 | 0 | 0.08396 | 0 | −0.06113 | 0 | 0.816754 |
| 3813 | −0.2639 | 0 | 0 | 0 | 0.079998 | 0 | −0.06554 | 0 | 0.812227 |
| 3814 | −0.24522 | 0 | 0 | 0 | 0.061623 | 0 | −0.07129 | 0 | 0.816714 |
| 3815 | −0.2681 | 0 | 0 | 0 | 0.060527 | 0 | −0.06777 | 0 | 0.801175 |
| 3816 | −0.26456 | 0 | 0 | 0 | 0.054344 | 0 | −0.07589 | 0 | 0.803304 |
| 3817 | −0.22226 | 0 | 0 | 0 | 0.108479 | 0 | 0 | −0.11768 | 0.812775 |
| 3818 | −0.21971 | 0 | 0 | 0 | 0.111207 | 0 | 0 | −0.13771 | 0.822577 |
| 3819 | −0.20365 | 0 | 0 | 0 | 0.089106 | 0 | 0 | −0.13444 | 0.821201 |
| 3820 | −0.22125 | 0 | 0 | 0 | 0.092758 | 0 | 0 | −0.14392 | 0.815833 |
| 3821 | −0.197 | 0 | 0 | 0 | 0.091945 | 0 | 0 | −0.17208 | 0.841485 |
| 3822 | −0.21732 | 0 | 0 | 0 | 0.057452 | 0 | 0 | 0 | 0.847393 |
| 3823 | −0.21774 | 0 | 0 | 0 | 0.082633 | 0 | 0 | 0 | 0.869975 |
| 3824 | −0.22158 | 0 | 0 | 0 | 0.052091 | 0 | 0 | 0 | 0.846855 |
| 3825 | −0.2433 | 0 | 0 | 0 | 0.05106 | 0 | 0 | 0 | 0.834591 |
| 3826 | −0.24038 | 0 | 0 | 0 | 0.044963 | 0 | 0 | 0 | 0.83501 |
| 3827 | −0.23256 | 0 | 0 | 0 | 0.054508 | 0 | 0 | 0 | 0.844804 |
| 3828 | −0.227 | 0 | 0 | 0 | 0.044712 | 0 | 0 | 0 | 0.845923 |
| 3829 | −0.22615 | 0 | 0 | 0 | 0.035091 | 0 | 0 | 0 | 0.838222 |
| 3830 | −0.21832 | 0 | 0 | 0 | 0.023602 | 0 | 0 | 0 | 0.843423 |
| 3831 | −0.2461 | 0 | 0 | 0 | 0.034641 | 0 | 0 | 0 | 0.820694 |
| 3832 | −5.12035 | 1.492011 | 0.062975 | 0 | 0 | 0 | 0 | 0 | 0.518863 |
| 3833 | −5.40535 | 1.38889 | 0.06932 | 0 | 0 | 0 | 0 | 0 | 0.607662 |
| 3834 | −5.28717 | 1.448935 | 0.066349 | 0 | 0 | 0 | 0 | 0 | 0.534616 |
| 3835 | −5.28656 | 1.430742 | 0.066256 | 0 | 0 | 0 | 0 | 0 | 0.516201 |
| 3836 | −5.27881 | 1.44799 | 0.066141 | 0 | 0 | 0 | 0 | 0 | 0.531286 |
| 3837 | −0.95133 | 1.225058 | 0 | 0.106978 | 0 | 0 | 0 | 0 | 0.802242 |
| 3838 | −0.95094 | 1.092023 | 0 | 0.070953 | 0 | 0 | 0 | 0 | 0.686976 |
| 3839 | −0.94488 | 1.155308 | 0 | 0.068694 | 0 | 0 | 0 | 0 | 0.728396 |
| 3840 | −0.96088 | 1.139628 | 0 | 0.08256 | 0 | 0 | 0 | 0 | 0.709704 |
| 3841 | −0.96458 | 1.161397 | 0 | 0.06735 | 0 | 0 | 0 | 0 | 0.71791 |
| 3842 | −0.97339 | 1.2394 | 0 | 0 | 0.004127 | 0 | 0 | 0 | 0.780587 |
| 3843 | −0.96293 | 1.102809 | 0 | 0 | 0.003859 | 0 | 0 | 0 | 0.677773 |
| 3844 | −0.95986 | 1.171107 | 0 | 0 | −0.01788 | 0 | 0 | 0 | 0.720987 |
| 3845 | −0.97976 | 1.157573 | 0 | 0 | −0.01643 | 0 | 0 | 0 | 0.69669 |
| 3846 | −0.97987 | 1.178811 | 0 | 0 | −0.02552 | 0 | 0 | 0 | 0.711855 |
| 3847 | −0.97002 | 1.257634 | 0 | 0 | 0 | −0.09428 | 0 | 0 | 0.785905 |
| 3848 | −0.9617 | 1.117495 | 0 | 0 | 0 | −0.07833 | 0 | 0 | 0.679441 |
| 3849 | −0.95134 | 1.181639 | 0 | 0 | 0 | −0.09255 | 0 | 0 | 0.726731 |
| 3850 | −0.96176 | 1.170884 | 0 | 0 | 0 | −0.12247 | 0 | 0 | 0.717263 |
| 3851 | −0.96946 | 1.188261 | 0 | 0 | 0 | −0.09707 | 0 | 0 | 0.718902 |
| 3852 | −0.98323 | 1.239213 | 0 | 0 | 0 | 0 | −0.04814 | 0 | 0.774782 |
| 3853 | −0.97383 | 1.103646 | 0 | 0 | 0 | 0 | −0.05686 | 0 | 0.671865 |
| 3854 | −0.96739 | 1.166086 | 0 | 0 | 0 | 0 | −0.06432 | 0 | 0.71655 |
| 3855 | −0.98721 | 1.152435 | 0 | 0 | 0 | 0 | −0.05961 | 0 | 0.690461 |
| 3856 | −0.98602 | 1.170971 | 0 | 0 | 0 | 0 | −0.06785 | 0 | 0.707287 |
| 3857 | −0.95743 | 1.243308 | 0 | 0 | 0 | 0 | 0 | −0.10351 | 0.773016 |
| 3858 | −0.93681 | 1.107671 | 0 | 0 | 0 | 0 | 0 | −0.12347 | 0.683575 |
| 3859 | −0.93026 | 1.171229 | 0 | 0 | 0 | 0 | 0 | −0.12443 | 0.726142 |
| 3860 | −0.94527 | 1.15525 | 0 | 0 | 0 | 0 | 0 | −0.12914 | 0.707517 |
| 3861 | −0.923 | 1.169666 | 0 | 0 | 0 | 0 | 0 | −0.15173 | 0.746252 |
| 3862 | −0.93899 | 1.212726 | 0 | 0 | 0 | 0 | 0 | 0 | 0.796186 |
| 3863 | −0.9104 | 1.117882 | 0 | 0 | 0 | 0 | 0 | 0 | 0.786923 |
| 3864 | −0.91571 | 1.074784 | 0 | 0 | 0 | 0 | 0 | 0 | 0.713519 |
| 3865 | −0.93313 | 1.06708 | 0 | 0 | 0 | 0 | 0 | 0 | 0.698122 |
| 3866 | −0.93561 | 1.084929 | 0 | 0 | 0 | 0 | 0 | 0 | 0.707106 |
| 3867 | −0.94796 | 1.192616 | 0 | 0 | 0 | 0 | 0 | 0 | 0.782722 |
| 3868 | −0.94733 | 1.218587 | 0 | 0 | 0 | 0 | 0 | 0 | 0.801054 |
| 3869 | −0.93485 | 1.13018 | 0 | 0 | 0 | 0 | 0 | 0 | 0.728844 |
| 3870 | −0.92828 | 1.144505 | 0 | 0 | 0 | 0 | 0 | 0 | 0.745764 |
| 3871 | −0.95985 | 1.144794 | 0 | 0 | 0 | 0 | 0 | 0 | 0.715642 |
| 3872 | −0.24923 | 0 | 0 | 0 | 0 | −0.0434 | −0.05211 | 0 | 0.820984 |
| 3873 | −0.25941 | 0 | 0 | 0 | 0 | −0.03687 | −0.05682 | 0 | 0.815835 |
| 3874 | −0.23584 | 0 | 0 | 0 | 0 | −0.04957 | −0.06418 | 0 | 0.826319 |
| 3875 | −0.2479 | 0 | 0 | 0 | 0 | −0.08556 | −0.0604 | 0 | 0.823977 |
| 3876 | −0.25341 | 0 | 0 | 0 | 0 | −0.0542 | −0.0699 | 0 | 0.815427 |
| 3877 | −0.22543 | 0 | 0 | 0 | 0 | −0.02461 | 0 | −0.09937 | 0.81564 |
| 3878 | −0.2248 | 0 | 0 | 0 | 0 | −0.01612 | 0 | −0.12042 | 0.823274 |
| 3879 | −0.20253 | 0 | 0 | 0 | 0 | −0.0286 | 0 | −0.11903 | 0.829441 |
| 3880 | −0.2123 | 0 | 0 | 0 | 0 | −0.064 | 0 | −0.12295 | 0.832762 |
| 3881 | −0.19667 | 0 | 0 | 0 | 0 | −0.0268 | 0 | −0.15513 | 0.848689 |
| 3882 | −0.20841 | 0 | 0 | 0 | 0 | −0.05772 | 0 | 0 | 0.855881 |
| 3883 | −0.21279 | 0 | 0 | 0 | 0 | −0.05571 | 0 | 0 | 0.87263 |
| 3884 | −0.21317 | 0 | 0 | 0 | 0 | −0.0559 | 0 | 0 | 0.855241 |
| 3885 | −0.22555 | 0 | 0 | 0 | 0 | −0.08952 | 0 | 0 | 0.853805 |
| 3886 | −0.23077 | 0 | 0 | 0 | 0 | −0.05835 | 0 | 0 | 0.844814 |
| 3887 | −0.21102 | 0 | 0 | 0 | 0 | −0.10188 | 0 | 0 | 0.867323 |
| 3888 | −0.21471 | 0 | 0 | 0 | 0 | −0.06831 | 0 | 0 | 0.857737 |
| 3889 | −0.20374 | 0 | 0 | 0 | 0 | −0.09798 | 0 | 0 | 0.862017 |
| 3890 | −0.20365 | 0 | 0 | 0 | 0 | −0.0725 | 0 | 0 | 0.857621 |
| 3891 | −0.22488 | 0 | 0 | 0 | 0 | −0.09257 | 0 | 0 | 0.844397 |
| 3892 | −0.23829 | 0 | 0 | 0 | 0 | 0 | −0.04842 | −0.10032 | 0.809697 |
| 3893 | −0.23618 | 0 | 0 | 0 | 0 | 0 | −0.05092 | −0.11961 | 0.818265 |
| 3894 | −0.21664 | 0 | 0 | 0 | 0 | 0 | −0.05896 | −0.11943 | 0.824127 |
| 3895 | −0.23474 | 0 | 0 | 0 | 0 | 0 | −0.05567 | −0.12724 | 0.816011 |
| 3896 | −0.21094 | 0 | 0 | 0 | 0 | 0 | −0.06382 | −0.15555 | 0.844521 |
| 3897 | −0.22789 | 0 | 0 | 0 | 0 | 0 | −0.05351 | 0 | 0.846075 |
| 3898 | −0.2284 | 0 | 0 | 0 | 0 | 0 | −0.03514 | 0 | 0.864049 |
| 3899 | −0.23179 | 0 | 0 | 0 | 0 | 0 | −0.05903 | 0 | 0.846248 |
| 3900 | −0.25318 | 0 | 0 | 0 | 0 | 0 | −0.05627 | 0 | 0.832913 |
| 3901 | −0.2509 | 0 | 0 | 0 | 0 | 0 | −0.06343 | 0 | 0.834758 |
| 3902 | −0.24234 | 0 | 0 | 0 | 0 | 0 | −0.04788 | 0 | 0.842221 |
| 3903 | −0.23723 | 0 | 0 | 0 | 0 | 0 | −0.05762 | 0 | 0.845565 |
| 3904 | −0.23579 | 0 | 0 | 0 | 0 | 0 | −0.06308 | 0 | 0.838163 |
| 3905 | −0.22815 | 0 | 0 | 0 | 0 | 0 | −0.07116 | 0 | 0.845183 |
| 3906 | −0.25663 | 0 | 0 | 0 | 0 | 0 | −0.06695 | 0 | 0.820866 |
| 3907 | −0.19686 | 0 | 0 | 0 | 0 | 0 | 0 | −0.11059 | 0.846265 |
| 3908 | −0.19844 | 0 | 0 | 0 | 0 | 0 | 0 | −0.11173 | 0.867896 |
| 3909 | −0.18896 | 0 | 0 | 0 | 0 | 0 | 0 | −0.13262 | 0.860687 |
| 3910 | −0.207 | 0 | 0 | 0 | 0 | 0 | 0 | −0.13803 | 0.853759 |
| 3911 | −0.18763 | 0 | 0 | 0 | 0 | 0 | 0 | −0.16416 | 0.875122 |
| 3912 | −0.20716 | 0 | 0 | 0 | 0 | 0 | 0 | −0.12014 | 0.850282 |
| 3913 | −0.18127 | 0 | 0 | 0 | 0 | 0 | 0 | −0.15512 | 0.876109 |
| 3914 | −0.18981 | 0 | 0 | 0 | 0 | 0 | 0 | −0.13778 | 0.856119 |
| 3915 | −0.16112 | 0 | 0 | 0 | 0 | 0 | 0 | −0.16874 | 0.886689 |
| 3916 | −0.19378 | 0 | 0 | 0 | 0 | 0 | 0 | −0.16182 | 0.861404 |
| 3917 | −4.61006 | 0 | 0.064136 | 0.203329 | 0.087205 | −0.06006 | −0.10595 | 0 | 0 |
| 3918 | −4.61874 | 0 | 0.065034 | 0.196767 | 0.105734 | −0.03094 | 0 | −0.1688 | 0 |
| 3919 | −4.65372 | 0 | 0.065318 | 0.218935 | 0.116602 | 0 | −0.10329 | −0.17037 | 0 |
| 3920 | −4.65852 | 0 | 0.065334 | 0.224565 | 0 | −0.01096 | −0.0909 | −0.15466 | 0 |
| 3921 | −4.43506 | 0 | 0.061829 | 0 | 0.137897 | −0.04165 | −0.07767 | −0.15654 | 0 |
| 3922 | −0.67896 | 0 | 0 | 0.086192 | 0.111972 | −0.00661 | −0.08963 | −0.13471 | 0 |
| 3923 | −6.13795 | 1.55127 | 0.074848 | 0.15625 | −0.00702 | −0.12053 | 0 | 0 | 0 |
| 3924 | −6.12794 | 1.526585 | 0.074475 | 0.17566 | −0.01747 | 0 | −0.09453 | 0 | 0 |
| 3925 | −6.18446 | 1.532114 | 0.076119 | 0.176481 | 0.011664 | 0 | 0 | −0.1687 | 0 |
| 3926 | −6.16749 | 1.548916 | 0.075134 | 0.170014 | 0 | −0.11782 | −0.09244 | 0 | 0 |
| 3927 | −6.21772 | 1.55387 | 0.076583 | 0.17411 | 0 | −0.08695 | 0 | −0.15623 | 0 |
| 3928 | −6.219 | 1.53498 | 0.076429 | 0.191848 | 0 | 0 | −0.08972 | −0.16401 | 0 |
| 3929 | −6.02647 | 1.561734 | 0.072753 | 0 | 0.02167 | −0.12682 | −0.07903 | 0 | 0 |
| 3930 | −6.05632 | 1.559359 | 0.073906 | 0 | 0.045729 | −0.09987 | 0 | −0.14928 | 0 |
| 3931 | −6.04243 | 1.541699 | 0.0735 | 0 | 0.040617 | 0 | −0.07551 | −0.15767 | 0 |
| 3932 | −6.08738 | 1.570871 | 0.074086 | 0 | 0 | −0.09234 | −0.0709 | −0.14157 | 0 |
| 3933 | −1.43761 | 1.288083 | 0 | 0.044026 | 0.011371 | −0.06972 | −0.08132 | 0 | 0 |
| 3934 | −1.39728 | 1.280463 | 0 | 0.037879 | 0.033419 | −0.05195 | 0 | −0.11832 | 0 |
| 3935 | −1.4063 | 1.268694 | 0 | 0.055553 | 0.032172 | 0 | −0.08032 | −0.12176 | 0 |
| 3936 | −1.4138 | 1.285836 | 0 | 0.05603 | 0 | −0.04353 | −0.07691 | −0.11229 | 0 |
| 3937 | −1.40902 | 1.282502 | 0 | 0 | 0.048615 | −0.05491 | −0.07402 | −0.11554 | 0 |
| 3938 | −0.2016 | 0 | 0 | 0 | 0 | 0 | 0 | 0 | 0.882879 |
| 3939 | −0.21424 | 0 | 0 | 0 | 0 | 0 | 0 | 0 | 0.880472 |
| 3940 | −0.20998 | 0 | 0 | 0 | 0 | 0 | 0 | 0 | 0.88246 |
| 3941 | −0.2172 | 0 | 0 | 0 | 0 | 0 | 0 | 0 | 0.860615 |
| 3942 | −0.21088 | 0 | 0 | 0 | 0 | 0 | 0 | 0 | 0.86437 |
| 3943 | −0.23605 | 0 | 0 | 0 | 0 | 0 | 0 | 0 | 0.84695 |
| 3944 | −0.21177 | 0 | 0 | 0 | 0 | 0 | 0 | 0 | 0.865186 |
| 3945 | −0.20327 | 0 | 0 | 0 | 0 | 0 | 0 | 0 | 0.870233 |
| 3946 | −0.22289 | 0 | 0 | 0 | 0 | 0 | 0 | 0 | 0.859508 |
| 3947 | −0.21601 | 0 | 0 | 0 | 0 | 0 | 0 | 0 | 0.85328 |
| 3948 | −3.44463 | 0 | 0.050402 | 0 | 0 | 0 | 0 | 0 | 0.597552 |
| 3949 | −3.8226 | 0 | 0.058095 | 0 | 0 | 0 | 0 | 0 | 0.765733 |
| 3950 | −3.61635 | 0 | 0.054028 | 0 | 0 | 0 | 0 | 0 | 0.667984 |
| 3951 | −3.64349 | 0 | 0.05428 | 0 | 0 | 0 | 0 | 0 | 0.666028 |
| 3952 | −3.63824 | 0 | 0.054249 | 0 | 0 | 0 | 0 | 0 | 0.662438 |
| 3953 | −0.22773 | 0 | 0 | 0.150372 | 0 | 0 | 0 | 0 | 0.850284 |
| 3954 | −0.24688 | 0 | 0 | 0.111852 | 0 | 0 | 0 | 0 | 0.829414 |
| 3955 | −0.22494 | 0 | 0 | 0.113547 | 0 | 0 | 0 | 0 | 0.835899 |
| 3956 | −0.24483 | 0 | 0 | 0.126767 | 0 | 0 | 0 | 0 | 0.82824 |
| 3957 | −0.24419 | 0 | 0 | 0.110968 | 0 | 0 | 0 | 0 | 0.821524 |
| 3958 | −0.24325 | 0 | 0 | 0 | 0.075316 | 0 | 0 | 0 | 0.821853 |
| 3959 | −0.25245 | 0 | 0 | 0 | 0.070775 | 0 | 0 | 0 | 0.817181 |
| 3960 | −0.23349 | 0 | 0 | 0 | 0.05158 | 0 | 0 | 0 | 0.820431 |
| 3961 | −0.25625 | 0 | 0 | 0 | 0.051237 | 0 | 0 | 0 | 0.806562 |
| 3962 | −0.25239 | 0 | 0 | 0 | 0.043795 | 0 | 0 | 0 | 0.806273 |
| 3963 | −0.97416 | 1.240448 | 0 | 0 | 0 | 0 | 0 | 0 | 0.780454 |
| 3964 | −0.96371 | 1.103876 | 0 | 0 | 0 | 0 | 0 | 0 | 0.677639 |
| 3965 | −0.95702 | 1.16639 | 0 | 0 | 0 | 0 | 0 | 0 | 0.720301 |
| 3966 | −0.97708 | 1.153236 | 0 | 0 | 0 | 0 | 0 | 0 | 0.696216 |
| 3967 | −0.97591 | 1.172048 | 0 | 0 | 0 | 0 | 0 | 0 | 0.710388 |
| 3968 | −0.23894 | 0 | 0 | 0 | 0 | −0.04479 | 0 | 0 | 0.825434 |
| 3969 | −0.24903 | 0 | 0 | 0 | 0 | −0.03864 | 0 | 0 | 0.820306 |
| 3970 | −0.22501 | 0 | 0 | 0 | 0 | −0.05112 | 0 | 0 | 0.829277 |
| 3971 | −0.23692 | 0 | 0 | 0 | 0 | −0.08723 | 0 | 0 | 0.828639 |
| 3972 | −0.24206 | 0 | 0 | 0 | 0 | −0.05561 | 0 | 0 | 0.81744 |
| 3973 | −0.25602 | 0 | 0 | 0 | 0 | 0 | −0.05296 | 0 | 0.817168 |
| 3974 | −0.26473 | 0 | 0 | 0 | 0 | 0 | −0.0576 | 0 | 0.813182 |
| 3975 | −0.24417 | 0 | 0 | 0 | 0 | 0 | −0.06505 | 0 | 0.820866 |
| 3976 | −0.26715 | 0 | 0 | 0 | 0 | 0 | −0.062 | 0 | 0.805148 |
| 3977 | −0.26311 | 0 | 0 | 0 | 0 | 0 | −0.07068 | 0 | 0.808209 |
| 3978 | −0.2287 | 0 | 0 | 0 | 0 | 0 | 0 | −0.10224 | 0.813416 |
| 3979 | −0.22665 | 0 | 0 | 0 | 0 | 0 | 0 | −0.12211 | 0.822259 |
| 3980 | −0.20652 | 0 | 0 | 0 | 0 | 0 | 0 | −0.12213 | 0.826549 |
| 3981 | −0.22463 | 0 | 0 | 0 | 0 | 0 | 0 | −0.12976 | 0.819817 |
| 3982 | −0.20038 | 0 | 0 | 0 | 0 | 0 | 0 | −0.15811 | 0.846065 |
| 3983 | −4.57292 | 0 | 0.063766 | 0.182608 | 0.069993 | −0.05982 | 0 | 0 | 0 |
| 3984 | −4.60104 | 0 | 0.063879 | 0.205707 | 0.073378 | 0 | −0.10593 | 0 | 0 |
| 3985 | −4.61676 | 0 | 0.064952 | 0.198308 | 0.099545 | 0 | 0 | −0.1723 | 0 |
| 3986 | −4.6107 | 0 | 0.064097 | 0.209174 | 0 | −0.04266 | −0.09688 | 0 | 0 |
| 3987 | −4.62913 | 0 | 0.065074 | 0.205642 | 0 | −0.01314 | 0 | −0.15806 | 0 |
| 3988 | −4.65752 | 0 | 0.065302 | 0.225037 | 0 | 0 | −0.0911 | −0.15611 | 0 |
| 3989 | −4.41183 | 0 | 0.060948 | 0 | 0.102287 | −0.06701 | −0.08225 | 0 | 0 |
| 3990 | −4.42442 | 0 | 0.061829 | 0 | 0.124605 | −0.04004 | 0 | −0.15898 | 0 |
| 3991 | −4.43021 | 0 | 0.06168 | 0 | 0.129726 | 0 | −0.07707 | −0.1609 | 0 |
| 3992 | −4.44038 | 0 | 0.06177 | 0 | 0 | −0.01899 | −0.06339 | −0.14191 | 0 |
| 3993 | −0.70679 | 0 | 0 | 0.079151 | 0.078951 | −0.02526 | −0.0924 | 0 | 0 |
| 3994 | −0.66639 | 0 | 0 | 0.071782 | 0.102195 | −0.00708 | 0 | −0.13667 | 0 |
| 3995 | −0.67958 | 0 | 0 | 0.086684 | 0.110544 | 0 | −0.08965 | −0.13527 | 0 |
| 3996 | −0.68561 | 0 | 0 | 0.098668 | 0 | 0.014101 | −0.08125 | −0.1216 | 0 |
| 3997 | −0.6784 | 0 | 0 | 0 | 0.122199 | −0.01251 | −0.08007 | −0.13217 | 0 |
| 3998 | −6.09849 | 1.529419 | 0.074181 | 0.161555 | −0.03171 | 0 | 0 | 0 | 0 |
| 3999 | −6.13578 | 1.549894 | 0.074829 | 0.155569 | 0 | −0.12184 | 0 | 0 | 0 |
| 4000 | −6.12254 | 1.522334 | 0.074427 | 0.174356 | 0 | 0 | −0.09616 | 0 | 0 |
| 4001 | −6.1886 | 1.534695 | 0.076153 | 0.1775 | 0 | 0 | 0 | −0.1673 | 0 |
| 4002 | −6.00703 | 1.561871 | 0.072611 | 0 | 0.01018 | −0.12694 | 0 | 0 | 0 |
| 4003 | −5.98074 | 1.538877 | 0.071975 | 0 | −0.00284 | 0 | −0.07926 | 0 | 0 |
| 4004 | −6.02529 | 1.541178 | 0.073396 | 0 | 0.029443 | 0 | 0 | −0.15955 | 0 |
| 4005 | −6.0316 | 1.566236 | 0.072785 | 0 | 0 | −0.12303 | −0.07713 | 0 | 0 |
| 4006 | −6.06867 | 1.568362 | 0.073975 | 0 | 0 | −0.09346 | 0 | −0.14444 | 0 |
| 4007 | −6.05354 | 1.55082 | 0.073571 | 0 | 0 | 0 | −0.07195 | −0.15261 | 0 |
| 4008 | −1.42909 | 1.292356 | 0 | 0.030467 | 0.002302 | −0.07113 | 0 | 0 | 0 |
| 4009 | −1.43893 | 1.276909 | 0 | 0.049402 | −0.00429 | 0 | −0.08206 | 0 | 0 |
| 4010 | −1.39731 | 1.27214 | 0 | 0.04205 | 0.023063 | 0 | 0 | −0.12295 | 0 |
| 4011 | −1.43944 | 1.290541 | 0 | 0.045453 | 0 | −0.06742 | −0.0805 | 0 | 0 |
| 4012 | −1.40364 | 1.28752 | 0 | 0.042033 | 0 | −0.04615 | 0 | −0.11438 | 0 |
| 4013 | −1.41235 | 1.276756 | 0 | 0.058845 | 0 | 0 | −0.07793 | −0.11728 | 0 |
| 4014 | −1.43987 | 1.29302 | 0 | 0 | 0.017296 | −0.07296 | −0.07639 | 0 | 0 |
| 4015 | −1.40001 | 1.284605 | 0 | 0 | 0.038772 | −0.05494 | 0 | −0.117 | 0 |
| 4016 | −1.40945 | 1.274175 | 0 | 0 | 0.038345 | 0 | −0.07394 | −0.12023 | 0 |
| 4017 | −1.41834 | 1.293723 | 0 | 0 | 0 | −0.04692 | −0.06996 | −0.10955 | 0 |
| 4018 | −0.21789 | 0 | 0 | 0 | 0 | 0 | 0 | 0 | 0.849983 |
| 4019 | −0.23305 | 0 | 0 | 0 | 0 | 0 | 0 | 0 | 0.847109 |
| 4020 | −0.22677 | 0 | 0 | 0 | 0 | 0 | 0 | 0 | 0.849212 |
| 4021 | −0.22543 | 0 | 0 | 0 | 0 | 0 | 0 | 0 | 0.841624 |
| 4022 | −0.21745 | 0 | 0 | 0 | 0 | 0 | 0 | 0 | 0.846581 |
| 4023 | −0.24549 | 0 | 0 | 0 | 0 | 0 | 0 | 0 | 0.824203 |
| 4024 | −0.22102 | 0 | 0 | 0 | 0 | 0 | 0 | 0 | 0.868218 |
| 4025 | −0.22196 | 0 | 0 | 0 | 0 | 0 | 0 | 0 | 0.849553 |
| 4026 | −0.24349 | 0 | 0 | 0 | 0 | 0 | 0 | 0 | 0.837314 |
| 4027 | −0.24024 | 0 | 0 | 0 | 0 | 0 | 0 | 0 | 0.838228 |
| 4028 | −4.5643 | 0 | 0.063515 | 0.184702 | 0.05609 | 0 | 0 | 0 | 0 |
| 4029 | −4.57916 | 0 | 0.063807 | 0.189065 | 0 | −0.04556 | 0 | 0 | 0 |
| 4030 | −4.60339 | 0 | 0.063903 | 0.210237 | 0 | 0 | −0.09788 | 0 | 0 |
| 4031 | −4.62789 | 0 | 0.065035 | 0.206094 | 0 | 0 | 0 | −0.15981 | 0 |
| 4032 | −4.40009 | 0 | 0.060933 | 0 | 0.088329 | −0.06564 | 0 | 0 | 0 |
| 4033 | −4.40002 | 0 | 0.060629 | 0 | 0.086958 | 0 | −0.08148 | 0 | 0 |
| 4034 | −4.42044 | 0 | 0.061696 | 0 | 0.1168 | 0 | 0 | −0.16315 | 0 |
| 4035 | −4.41396 | 0 | 0.060914 | 0 | 0 | −0.04654 | −0.07123 | 0 | 0 |
| 4036 | −4.43292 | 0 | 0.061799 | 0 | 0 | −0.01937 | 0 | −0.14522 | 0 |
| 4037 | −4.43771 | 0 | 0.061698 | 0 | 0 | 0 | −0.06347 | −0.14424 | 0 |
| 4038 | −0.69417 | 0 | 0 | 0.064325 | 0.068359 | −0.02567 | 0 | 0 | 0 |
| 4039 | −0.70952 | 0 | 0 | 0.080757 | 0.072812 | 0 | −0.09247 | 0 | 0 |
| 4040 | −0.66704 | 0 | 0 | 0.072293 | 0.100655 | 0 | 0 | −0.13727 | 0 |
| 4041 | −0.70976 | 0 | 0 | 0.088735 | 0 | −0.00814 | −0.08617 | 0 | 0 |
| 4042 | −0.67355 | 0 | 0 | 0.084108 | 0 | 0.011968 | 0 | −0.12454 | 0 |
| 4043 | −0.68444 | 0 | 0 | 0.097935 | 0 | 0 | −0.08097 | −0.12006 | 0 |
| 4044 | −0.70585 | 0 | 0 | 0 | 0.08883 | −0.02986 | −0.08372 | 0 | 0 |
| 4045 | −0.66701 | 0 | 0 | 0 | 0.111493 | −0.01191 | 0 | −0.13441 | 0 |
| 4046 | −0.67955 | 0 | 0 | 0 | 0.119594 | 0 | −0.08 | −0.13318 | 0 |
| 4047 | −6.08687 | 1.521958 | 0.074071 | 0.158471 | 0 | 0 | 0 | 0 | 0 |
| 4048 | −5.96128 | 1.538397 | 0.071838 | 0 | −0.01467 | 0 | 0 | 0 | 0 |
| 4049 | −6.00976 | 1.563935 | 0.07263 | 0 | 0 | −0.12512 | 0 | 0 | 0 |
| 4050 | −5.97993 | 1.538189 | 0.071969 | 0 | 0 | 0 | −0.07952 | 0 | 0 |
| 4051 | −6.0342 | 1.54773 | 0.073456 | 0 | 0 | 0 | 0 | −0.15578 | 0 |
| 4052 | −1.43014 | 1.2808 | 0 | 0.035634 | −0.01392 | 0 | 0 | 0 | 0 |
| 4053 | −1.42947 | 1.29284 | 0 | 0.030782 | 0 | −0.07065 | 0 | 0 | 0 |
| 4054 | −1.43823 | 1.275797 | 0 | 0.048905 | 0 | 0 | −0.08239 | 0 | 0 |
| 4055 | −1.40183 | 1.277833 | 0 | 0.044686 | 0 | 0 | 0 | −0.11972 | 0 |
| 4056 | −1.43106 | 1.295655 | 0 | 0 | 0.006863 | −0.07329 | 0 | 0 | 0 |
| 4057 | −1.44141 | 1.281755 | 0 | 0 | 0.001618 | 0 | −0.07647 | 0 | 0 |
| 4058 | −1.40029 | 1.276178 | 0 | 0 | 0.028388 | 0 | 0 | −0.12173 | 0 |
| 4059 | −1.44279 | 1.297048 | 0 | 0 | 0 | −0.06957 | −0.07487 | 0 | 0 |
| 4060 | −1.40783 | 1.293439 | 0 | 0 | 0 | −0.04847 | 0 | −0.11217 | 0 |
| 4061 | −1.41692 | 1.284247 | 0 | 0 | 0 | 0 | −0.07062 | −0.11473 | 0 |
| 4062 | −0.68566 | 0 | 0 | 0 | 0 | 0.009572 | −0.06908 | −0.11717 | 0 |
| 4063 | −0.25445 | 0 | 0 | 0 | 0 | 0 | 0 | 0 | 0.817555 |
| 4064 | −0.2458 | 0 | 0 | 0 | 0 | 0 | 0 | 0 | 0.821538 |
| 4065 | −0.23348 | 0 | 0 | 0 | 0 | 0 | 0 | 0 | 0.823678 |
| 4066 | −0.2563 | 0 | 0 | 0 | 0 | 0 | 0 | 0 | 0.809548 |
| 4067 | −0.25189 | 0 | 0 | 0 | 0 | 0 | 0 | 0 | 0.810099 |
| 4068 | −4.57094 | 0 | 0.063595 | 0.189725 | 0 | 0 | 0 | 0 | 0 |
| 4069 | −4.38948 | 0 | 0.060634 | 0 | 0.073323 | 0 | 0 | 0 | 0 |
| 4070 | −4.40486 | 0 | 0.060925 | 0 | 0 | −0.0477 | 0 | 0 | 0 |
| 4071 | −4.4046 | 0 | 0.060677 | 0 | 0 | 0 | −0.07178 | 0 | 0 |
| 4072 | −4.43042 | 0 | 0.061725 | 0 | 0 | 0 | 0 | −0.14761 | 0 |
| 4073 | −0.69693 | 0 | 0 | 0.065899 | 0.062071 | 0 | 0 | 0 | 0 |
| 4074 | −0.69749 | 0 | 0 | 0.073293 | 0 | −0.01067 | 0 | 0 | 0 |
| 4075 | −0.71062 | 0 | 0 | 0.08903 | 0 | 0 | −0.08635 | 0 | 0 |
| 4076 | −0.6726 | 0 | 0 | 0.083542 | 0 | 0 | 0 | −0.12323 | 0 |
| 4077 | −0.69436 | 0 | 0 | 0 | 0.077092 | −0.02933 | 0 | 0 | 0 |
| 4078 | −0.70906 | 0 | 0 | 0 | 0.081802 | 0 | −0.08356 | 0 | 0 |
| 4079 | −0.66811 | 0 | 0 | 0 | 0.109004 | 0 | 0 | −0.13537 | 0 |
| 4080 | −5.95661 | 1.534886 | 0.071795 | 0 | 0 | 0 | 0 | 0 | 0 |
| 4081 | −1.42775 | 1.27725 | 0 | 0.033841 | 0 | 0 | 0 | 0 | 0 |
| 4082 | −1.4324 | 1.284179 | 0 | 0 | −0.00911 | 0 | 0 | 0 | 0 |
| 4083 | −1.43228 | 1.297226 | 0 | 0 | 0 | −0.07192 | 0 | 0 | 0 |
| 4084 | −1.44169 | 1.282196 | 0 | 0 | 0 | 0 | −0.07632 | 0 | 0 |
| 4085 | −1.40615 | 1.283579 | 0 | 0 | 0 | 0 | 0 | −0.1176 | 0 |
| 4086 | −0.70911 | 0 | 0 | 0 | 0 | −0.01092 | −0.07525 | 0 | 0 |
| 4087 | −0.67514 | 0 | 0 | 0 | 0 | 0.008392 | 0 | −0.12042 | 0 |
| 4088 | −0.68486 | 0 | 0 | 0 | 0 | 0 | −0.06896 | −0.11618 | 0 |
| 4089 | −4.39568 | 0 | 0.060691 | 0 | 0 | 0 | 0 | 0 | 0 |
| 4090 | −0.69857 | 0 | 0 | 0.073626 | 0 | 0 | 0 | 0 | 0 |
| 4091 | −0.69752 | 0 | 0 | 0 | 0.070149 | 0 | 0 | 0 | 0 |
| 4092 | −1.43075 | 1.281711 | 0 | 0 | 0 | 0 | 0 | 0 | 0 |
| 4093 | −0.69825 | 0 | 0 | 0 | 0 | −0.01267 | 0 | 0 | 0 |
| 4094 | −0.71024 | 0 | 0 | 0 | 0 | 0 | −0.07543 | 0 | 0 |
| 4095 | −0.67447 | 0 | 0 | 0 | 0 | 0 | 0 | −0.11954 | 0 |

| | Intercept.Low | Beta.AdipA1 | Beta.NTproBNP | Beta.PIGF | Beta.A8MPO | Beta.A12TNF | NumVar | AICc | AUC |
|---|---|---|---|---|---|---|---|---|---|
| 1 | −0.38017 | −0.47149 | 0.43946 | 0.184707 | 0.175726 | 0.194494 | 15 | 496.9587 | 0.810571 |
| 2 | −0.38017 | −0.47149 | 0.43946 | 0.184707 | 0.175726 | 0.194494 | 14 | 526.3189 | 0.775661 |
| 3 | −0.3784 | −0.48038 | 0.429094 | 0.184036 | 0.235336 | 0 | 14 | 497.7061 | 0.806529 |
| 4 | −0.37944 | −0.47686 | 0.432955 | 0.204998 | 0 | 0.247828 | 14 | 496.1622 | 0.808305 |
| 5 | −0.37057 | −0.48826 | 0.469023 | 0 | 0.198485 | 0.194003 | 14 | 496.1191 | 0.809999 |
| 6 | −0.36821 | −0.31823 | 0 | 0.248938 | 0.16128 | 0.17389 | 14 | 501.6984 | 0.801894 |
| 7 | −0.36844 | 0 | 0.31303 | 0.214129 | 0.186465 | 0.210249 | 14 | 506.4877 | 0.797056 |
| 8 | −0.38017 | −0.47149 | 0.43946 | 0.184707 | 0.175726 | 0.194494 | 14 | 500.6472 | 0.801323 |
| 9 | −0.38017 | −0.47149 | 0.43946 | 0.184707 | 0.175726 | 0.194494 | 14 | 494.8548 | 0.809734 |
| 10 | −0.38017 | −0.47149 | 0.43946 | 0.184707 | 0.175726 | 0.194494 | 14 | 495.7586 | 0.809101 |
| 11 | −0.38017 | −0.47149 | 0.43946 | 0.184707 | 0.175726 | 0.194494 | 14 | 494.5644 | 0.81053 |
| 12 | −0.38017 | −0.47149 | 0.43946 | 0.184707 | 0.175726 | 0.194494 | 14 | 499.4317 | 0.804242 |
| 13 | −0.38017 | −0.47149 | 0.43946 | 0.184707 | 0.175726 | 0.194494 | 14 | 536.7701 | 0.760044 |
| 14 | −0.3784 | −0.48038 | 0.429094 | 0.184036 | 0.235336 | 0 | 13 | 527.7625 | 0.77115 |
| 15 | −0.37944 | −0.47686 | 0.432955 | 0.204998 | 0 | 0.247828 | 13 | 527.0351 | 0.773436 |
| 16 | −0.37057 | −0.48826 | 0.469023 | 0 | 0.198485 | 0.194003 | 13 | 526.012 | 0.771333 |
| 17 | −0.36821 | −0.31823 | 0 | 0.248938 | 0.16128 | 0.17389 | 13 | 530.4349 | 0.769864 |
| 18 | −0.36844 | 0 | 0.31303 | 0.214129 | 0.186465 | 0.210249 | 13 | 542.1632 | 0.750939 |
| 19 | −0.38017 | −0.47149 | 0.43946 | 0.184707 | 0.175726 | 0.194494 | 13 | 530.8114 | 0.76525 |
| 20 | −0.38017 | −0.47149 | 0.43946 | 0.184707 | 0.175726 | 0.194494 | 13 | 524.4468 | 0.775192 |
| 21 | −0.38017 | −0.47149 | 0.43946 | 0.184707 | 0.175726 | 0.194494 | 13 | 524.6732 | 0.774988 |
| 22 | −0.38017 | −0.47149 | 0.43946 | 0.184707 | 0.175726 | 0.194494 | 13 | 524.6262 | 0.77413 |
| 23 | −0.38017 | −0.47149 | 0.43946 | 0.184707 | 0.175726 | 0.194494 | 13 | 530.381 | 0.767148 |
| 24 | −0.38017 | −0.47149 | 0.43946 | 0.184707 | 0.175726 | 0.194494 | 13 | 556.108 | 0.722481 |
| 25 | −0.37665 | −0.49016 | 0.415897 | 0.213769 | 0 | 0 | 13 | 498.7704 | 0.803813 |
| 26 | −0.3692 | −0.49878 | 0.458935 | 0 | 0.257995 | 0 | 13 | 496.8199 | 0.807039 |
| 27 | −0.36789 | −0.4958 | 0.464972 | 0 | 0 | 0.255069 | 13 | 495.7233 | 0.806957 |
| 28 | −0.36683 | −0.32775 | 0 | 0.247282 | 0.215748 | 0 | 13 | 502.2371 | 0.798342 |
| 29 | −0.36769 | −0.32552 | 0 | 0.266766 | 0 | 0.223296 | 13 | 500.701 | 0.801343 |
| 30 | −0.35346 | −0.32601 | 0 | 0 | 0.190682 | 0.171912 | 13 | 502.1243 | 0.799669 |
| 31 | −0.36573 | 0 | 0.297911 | 0.217151 | 0.249518 | 0 | 13 | 507.4843 | 0.794239 |
| 32 | −0.36751 | 0 | 0.304766 | 0.234822 | 0 | 0.26618 | 13 | 505.8791 | 0.795362 |
| 33 | −0.35702 | 0 | 0.341819 | 0 | 0.21209 | 0.213821 | 13 | 506.2119 | 0.795831 |
| 34 | −0.36116 | 0 | 0 | 0.256551 | 0.172681 | 0.188346 | 13 | 507.0009 | 0.7943 |
| 35 | −0.3784 | −0.48038 | 0.429094 | 0.184036 | 0.235336 | 0 | 13 | 500.1127 | 0.799935 |
| 36 | −0.37944 | −0.47686 | 0.432955 | 0.204998 | 0 | 0.247828 | 13 | 499.9563 | 0.801057 |
| 37 | −0.37057 | −0.48826 | 0.469023 | 0 | 0.198485 | 0.194003 | 13 | 499.7069 | 0.80069 |
| 38 | −0.36821 | −0.31823 | 0 | 0.248938 | 0.16128 | 0.17389 | 13 | 506.0869 | 0.793504 |
| 39 | −0.36844 | 0 | 0.31303 | 0.214129 | 0.186465 | 0.210249 | 13 | 509.1258 | 0.791299 |
| 40 | −0.3784 | −0.48038 | 0.429094 | 0.184036 | 0.235336 | 0 | 13 | 495.6305 | 0.806263 |
| 41 | −0.37944 | −0.47686 | 0.432955 | 0.204998 | 0 | 0.247828 | 13 | 494.1308 | 0.808693 |
| 42 | −0.37057 | −0.48826 | 0.469023 | 0 | 0.198485 | 0.194003 | 13 | 494.0932 | 0.808366 |
| 43 | −0.36821 | −0.31823 | 0 | 0.248938 | 0.16128 | 0.17389 | 13 | 499.7961 | 0.80165 |
| 44 | −0.36844 | 0 | 0.31303 | 0.214129 | 0.186465 | 0.210249 | 13 | 504.6509 | 0.796423 |
| 45 | −0.38017 | −0.47149 | 0.43946 | 0.184707 | 0.175726 | 0.194494 | 13 | 498.6243 | 0.801568 |
| 46 | −0.3784 | −0.48038 | 0.429094 | 0.184036 | 0.235336 | 0 | 13 | 496.6614 | 0.805794 |
| 47 | −0.37944 | −0.47686 | 0.432955 | 0.204998 | 0 | 0.247828 | 13 | 494.5587 | 0.808427 |
| 48 | −0.37057 | −0.48826 | 0.469023 | 0 | 0.198485 | 0.194003 | 13 | 494.9389 | 0.808386 |
| 49 | −0.36821 | −0.31823 | 0 | 0.248938 | 0.16128 | 0.17389 | 13 | 500.2356 | 0.801343 |
| 50 | −0.36844 | 0 | 0.31303 | 0.214129 | 0.186465 | 0.210249 | 13 | 505.2323 | 0.795239 |
| 51 | −0.38017 | −0.47149 | 0.43946 | 0.184707 | 0.175726 | 0.194494 | 13 | 500.5542 | 0.800139 |
| 52 | −0.38017 | −0.47149 | 0.43946 | 0.184707 | 0.175726 | 0.194494 | 13 | 493.7576 | 0.808284 |
| 53 | −0.3784 | −0.48038 | 0.429094 | 0.184036 | 0.235336 | 0 | 13 | 495.4136 | 0.806753 |
| 54 | −0.37944 | −0.47686 | 0.432955 | 0.204998 | 0 | 0.247828 | 13 | 493.7939 | 0.80855 |
| 55 | −0.37057 | −0.48826 | 0.469023 | 0 | 0.198485 | 0.194003 | 13 | 493.8142 | 0.809836 |
| 56 | −0.36821 | −0.31823 | 0 | 0.248938 | 0.16128 | 0.17389 | 13 | 499.3383 | 0.801874 |
| 57 | −0.36844 | 0 | 0.31303 | 0.214129 | 0.186465 | 0.210249 | 13 | 504.0958 | 0.797138 |
| 58 | −0.38017 | −0.47149 | 0.43946 | 0.184707 | 0.175726 | 0.194494 | 13 | 498.2373 | 0.801527 |
| 59 | −0.38017 | −0.47149 | 0.43946 | 0.184707 | 0.175726 | 0.194494 | 13 | 492.4697 | 0.809428 |
| 60 | −0.38017 | −0.47149 | 0.43946 | 0.184707 | 0.175726 | 0.194494 | 13 | 493.3464 | 0.808958 |
| 61 | −0.3784 | −0.48038 | 0.429094 | 0.184036 | 0.235336 | 0 | 13 | 500.0748 | 0.801405 |
| 62 | −0.37944 | −0.47686 | 0.432955 | 0.204998 | 0 | 0.247828 | 13 | 498.4185 | 0.802997 |
| 63 | −0.37057 | −0.48826 | 0.469023 | 0 | 0.198485 | 0.194003 | 13 | 498.8505 | 0.802446 |
| 64 | −0.36821 | −0.31823 | 0 | 0.248938 | 0.16128 | 0.17389 | 13 | 503.2676 | 0.795933 |
| 65 | −0.36844 | 0 | 0.31303 | 0.214129 | 0.186465 | 0.210249 | 13 | 508.1868 | 0.791258 |
| 66 | −0.38017 | −0.47149 | 0.43946 | 0.184707 | 0.175726 | 0.194494 | 13 | 502.2823 | 0.797158 |
| 67 | −0.38017 | −0.47149 | 0.43946 | 0.184707 | 0.175726 | 0.194494 | 13 | 497.1735 | 0.803977 |
| 68 | −0.38017 | −0.47149 | 0.43946 | 0.184707 | 0.175726 | 0.194494 | 13 | 498.5734 | 0.802446 |
| 69 | −0.38017 | −0.47149 | 0.43946 | 0.184707 | 0.175726 | 0.194494 | 13 | 497.1818 | 0.803058 |
| 70 | −0.3784 | −0.48038 | 0.429094 | 0.184036 | 0.235336 | 0 | 13 | 538.4468 | 0.755165 |
| 71 | −0.37944 | −0.47686 | 0.432955 | 0.204998 | 0 | 0.247828 | 13 | 536.6147 | 0.757533 |
| 72 | −0.37057 | −0.48826 | 0.469023 | 0 | 0.198485 | 0.194003 | 13 | 536.2558 | 0.757125 |
| 73 | −0.36821 | −0.31823 | 0 | 0.248938 | 0.16128 | 0.17389 | 13 | 550.2259 | 0.738588 |
| 74 | −0.36844 | 0 | 0.31303 | 0.214129 | 0.186465 | 0.210249 | 13 | 541.8345 | 0.751899 |
| 75 | −0.38017 | −0.47149 | 0.43946 | 0.184707 | 0.175726 | 0.194494 | 13 | 538.5433 | 0.753532 |
| 76 | −0.38017 | −0.47149 | 0.43946 | 0.184707 | 0.175726 | 0.194494 | 13 | 534.5904 | 0.758186 |
| 77 | −0.38017 | −0.47149 | 0.43946 | 0.184707 | 0.175726 | 0.194494 | 13 | 535.515 | 0.756247 |
| 78 | −0.38017 | −0.47149 | 0.43946 | 0.184707 | 0.175726 | 0.194494 | 13 | 534.4151 | 0.759962 |
| 79 | −0.38017 | −0.47149 | 0.43946 | 0.184707 | 0.175726 | 0.194494 | 13 | 535.9064 | 0.756839 |
| 80 | −0.37665 | −0.49016 | 0.415897 | 0.213769 | 0 | 0 | 12 | 531.3024 | 0.765168 |
| 81 | −0.3692 | −0.49878 | 0.458935 | 0 | 0.257995 | 0 | 12 | 527.3884 | 0.767863 |
| 82 | −0.36789 | −0.4958 | 0.464972 | 0 | 0 | 0.255069 | 12 | 527.4271 | 0.768108 |
| 83 | −0.36683 | −0.32775 | 0 | 0.247282 | 0.215748 | 0 | 12 | 531.7313 | 0.76476 |
| 84 | −0.36769 | −0.32552 | 0 | 0.266766 | 0 | 0.223296 | 12 | 530.7524 | 0.768537 |
| 85 | −0.35346 | −0.32601 | 0 | 0 | 0.190682 | 0.171912 | 12 | 531.7124 | 0.763208 |
| 86 | −0.36573 | 0 | 0.297911 | 0.217151 | 0.249518 | 0 | 12 | 544.1023 | 0.744713 |
| 87 | −0.36751 | 0 | 0.304766 | 0.234822 | 0 | 0.26618 | 12 | 543.2999 | 0.748571 |
| 88 | −0.35702 | 0 | 0.341819 | 0 | 0.21209 | 0.213821 | 12 | 542.6464 | 0.746836 |
| 89 | −0.36116 | 0 | 0 | 0.256551 | 0.172681 | 0.188346 | 12 | 541.1198 | 0.750633 |
| 90 | −0.3784 | −0.48038 | 0.429094 | 0.184036 | 0.235336 | 0 | 12 | 530.3822 | 0.763535 |
| 91 | −0.37944 | −0.47686 | 0.432955 | 0.204998 | 0 | 0.247828 | 12 | 531.7492 | 0.762412 |
| 92 | −0.37057 | −0.48826 | 0.469023 | 0 | 0.198485 | 0.194003 | 12 | 530.4268 | 0.761657 |
| 93 | −0.36821 | −0.31823 | 0 | 0.248938 | 0.16128 | 0.17389 | 12 | 535.9203 | 0.758064 |
| 94 | −0.36844 | 0 | 0.31303 | 0.214129 | 0.186465 | 0.210249 | 12 | 545.6417 | 0.74263 |
| 95 | −0.3784 | −0.48038 | 0.429094 | 0.184036 | 0.235336 | 0 | 12 | 525.9034 | 0.771476 |
| 96 | −0.37944 | −0.47686 | 0.432955 | 0.204998 | 0 | 0.247828 | 12 | 525.267 | 0.772905 |
| 97 | −0.37057 | −0.48826 | 0.469023 | 0 | 0.198485 | 0.194003 | 12 | 524.2504 | 0.771885 |
| 98 | −0.36821 | −0.31823 | 0 | 0.248938 | 0.16128 | 0.17389 | 12 | 528.8143 | 0.769435 |
| 99 | −0.36844 | 0 | 0.31303 | 0.214129 | 0.186465 | 0.210249 | 12 | 540.6887 | 0.750674 |
| 100 | −0.38017 | −0.47149 | 0.43946 | 0.184707 | 0.175726 | 0.194494 | 12 | 529.0153 | 0.764719 |
| 101 | −0.3784 | −0.48038 | 0.429094 | 0.184036 | 0.235336 | 0 | 12 | 526.2754 | 0.770109 |
| 102 | −0.37944 | −0.47686 | 0.432955 | 0.204998 | 0 | 0.247828 | 12 | 525.0102 | 0.773069 |
| 103 | −0.37057 | −0.48826 | 0.469023 | 0 | 0.198485 | 0.194003 | 12 | 524.3623 | 0.770884 |
| 104 | −0.36821 | −0.31823 | 0 | 0.248938 | 0.16128 | 0.17389 | 12 | 528.6602 | 0.769027 |
| 105 | −0.36844 | 0 | 0.31303 | 0.214129 | 0.186465 | 0.210249 | 12 | 540.4513 | 0.749837 |
| 106 | −0.38017 | −0.47149 | 0.43946 | 0.184707 | 0.175726 | 0.194494 | 12 | 530.1375 | 0.763188 |
| 107 | −0.38017 | −0.47149 | 0.43946 | 0.184707 | 0.175726 | 0.194494 | 12 | 522.8739 | 0.774212 |
| 108 | −0.3784 | −0.48038 | 0.429094 | 0.184036 | 0.235336 | 0 | 12 | 526.3108 | 0.769721 |
| 109 | −0.37944 | −0.47686 | 0.432955 | 0.204998 | 0 | 0.247828 | 12 | 525.3539 | 0.771946 |
| 110 | −0.37057 | −0.48826 | 0.469023 | 0 | 0.198485 | 0.194003 | 12 | 524.5836 | 0.769843 |
| 111 | −0.36821 | −0.31823 | 0 | 0.248938 | 0.16128 | 0.17389 | 12 | 528.7097 | 0.769047 |
| 112 | −0.36844 | 0 | 0.31303 | 0.214129 | 0.186465 | 0.210249 | 12 | 540.4519 | 0.751082 |
| 113 | −0.38017 | −0.47149 | 0.43946 | 0.184707 | 0.175726 | 0.194494 | 12 | 528.7155 | 0.763474 |
| 114 | −0.38017 | −0.47149 | 0.43946 | 0.184707 | 0.175726 | 0.194494 | 12 | 522.6521 | 0.773987 |
| 115 | −0.38017 | −0.47149 | 0.43946 | 0.184707 | 0.175726 | 0.194494 | 12 | 522.7801 | 0.773293 |
| 116 | −0.3784 | −0.48038 | 0.429094 | 0.184036 | 0.235336 | 0 | 12 | 531.8083 | 0.761677 |
| 117 | −0.37944 | −0.47686 | 0.432955 | 0.204998 | 0 | 0.247828 | 12 | 530.7406 | 0.765801 |
| 118 | −0.37057 | −0.48826 | 0.469023 | 0 | 0.198485 | 0.194003 | 12 | 530.538 | 0.761432 |
| 119 | −0.36821 | −0.31823 | 0 | 0.248938 | 0.16128 | 0.17389 | 12 | 533.4287 | 0.762759 |
| 120 | −0.36844 | 0 | 0.31303 | 0.214129 | 0.186465 | 0.210249 | 12 | 545.4058 | 0.740262 |
| 121 | −0.38017 | −0.47149 | 0.43946 | 0.184707 | 0.175726 | 0.194494 | 12 | 534.0036 | 0.758268 |
| 122 | −0.38017 | −0.47149 | 0.43946 | 0.184707 | 0.175726 | 0.194494 | 12 | 528.1783 | 0.767373 |
| 123 | −0.38017 | −0.47149 | 0.43946 | 0.184707 | 0.175726 | 0.194494 | 12 | 529.0409 | 0.765719 |
| 124 | −0.38017 | −0.47149 | 0.43946 | 0.184707 | 0.175726 | 0.194494 | 12 | 529.0871 | 0.765597 |
| 125 | −0.3784 | −0.48038 | 0.429094 | 0.184036 | 0.235336 | 0 | 12 | 558.3567 | 0.718296 |
| 126 | −0.37944 | −0.47686 | 0.432955 | 0.204998 | 0 | 0.247828 | 12 | 557.1061 | 0.71799 |
| 127 | −0.37057 | −0.48826 | 0.469023 | 0 | 0.198485 | 0.194003 | 12 | 556.2439 | 0.720296 |
| 128 | −0.36821 | −0.31823 | 0 | 0.248938 | 0.16128 | 0.17389 | 12 | 569.2139 | 0.702964 |
| 129 | −0.36844 | 0 | 0.31303 | 0.214129 | 0.186465 | 0.210249 | 12 | 567.618 | 0.70323 |
| 130 | −0.38017 | −0.47149 | 0.43946 | 0.184707 | 0.175726 | 0.194494 | 12 | 559.0265 | 0.715193 |
| 131 | −0.38017 | −0.47149 | 0.43946 | 0.184707 | 0.175726 | 0.194494 | 12 | 554.1352 | 0.719949 |
| 132 | −0.38017 | −0.47149 | 0.43946 | 0.184707 | 0.175726 | 0.194494 | 12 | 554.6064 | 0.719582 |
| 133 | −0.38017 | −0.47149 | 0.43946 | 0.184707 | 0.175726 | 0.194494 | 12 | 554.3672 | 0.720562 |
| 134 | −0.38017 | −0.47149 | 0.43946 | 0.184707 | 0.175726 | 0.194494 | 12 | 556.3438 | 0.721868 |
| 135 | −0.34604 | 0 | 0 | 0 | 0.20186 | 0.189915 | 12 | 507.5383 | 0.792075 |
| 136 | −0.36484 | −0.51223 | 0.448932 | 0 | 0 | 0 | 12 | 498.4676 | 0.802915 |
| 137 | −0.36535 | −0.34122 | 0 | 0.272545 | 0 | 0 | 12 | 502.9396 | 0.796199 |
| 138 | −0.35247 | −0.33749 | 0 | 0 | 0.244058 | 0 | 12 | 502.5501 | 0.796382 |
| 139 | −0.3509 | −0.33426 | 0 | 0 | 0 | 0.230316 | 12 | 501.5906 | 0.797812 |
| 140 | −0.3635 | 0 | 0.280897 | 0.247949 | 0 | 0 | 12 | 508.76 | 0.790074 |
| 141 | −0.35435 | 0 | 0.326983 | 0 | 0.276248 | 0 | 12 | 507.2209 | 0.792095 |
| 142 | −0.35429 | 0 | 0.335115 | 0 | 0 | 0.278056 | 12 | 506.0588 | 0.792994 |
| 143 | −0.35918 | 0 | 0 | 0.257296 | 0.231108 | 0 | 12 | 507.7044 | 0.790626 |
| 144 | −0.36046 | 0 | 0 | 0.27483 | 0 | 0.241127 | 12 | 506.2244 | 0.79185 |
| 145 | −0.37665 | −0.49016 | 0.415897 | 0.213769 | 0 | 0 | 12 | 500.8475 | 0.796525 |
| 146 | −0.3692 | −0.49878 | 0.458935 | 0 | 0.257995 | 0 | 12 | 499.1567 | 0.79822 |
| 147 | −0.36789 | −0.4958 | 0.464972 | 0 | 0 | 0.255069 | 12 | 499.4132 | 0.798853 |
| 148 | −0.36683 | −0.32775 | 0 | 0.247282 | 0.215748 | 0 | 12 | 505.378 | 0.790768 |
| 149 | −0.36769 | −0.32552 | 0 | 0.266766 | 0 | 0.223296 | 12 | 505.1816 | 0.791605 |
| 150 | −0.35346 | −0.32601 | 0 | 0 | 0.190682 | 0.171912 | 12 | 506.3154 | 0.790156 |
| 151 | −0.36573 | 0 | 0.297911 | 0.217151 | 0.249518 | 0 | 12 | 509.0179 | 0.787033 |
| 152 | −0.36751 | 0 | 0.304766 | 0.234822 | 0 | 0.26618 | 12 | 508.5901 | 0.78887 |
| 153 | −0.35702 | 0 | 0.341819 | 0 | 0.21209 | 0.213821 | 12 | 508.7398 | 0.789094 |
| 154 | −0.36116 | 0 | 0 | 0.256551 | 0.172681 | 0.188346 | 12 | 510.2861 | 0.785991 |
| 155 | −0.37665 | −0.49016 | 0.415897 | 0.213769 | 0 | 0 | 12 | 496.7782 | 0.80414 |
| 156 | −0.3692 | −0.49878 | 0.458935 | 0 | 0.257995 | 0 | 12 | 494.8187 | 0.805916 |
| 157 | −0.36789 | −0.4958 | 0.464972 | 0 | 0 | 0.255069 | 12 | 493.7799 | 0.80661 |
| 158 | −0.36683 | −0.32775 | 0 | 0.247282 | 0.215748 | 0 | 12 | 500.3623 | 0.798281 |
| 159 | −0.36769 | −0.32552 | 0 | 0.266766 | 0 | 0.223296 | 12 | 498.8715 | 0.800384 |
| 160 | −0.35346 | −0.32601 | 0 | 0 | 0.190682 | 0.171912 | 12 | 500.3216 | 0.799036 |
| 161 | −0.36573 | 0 | 0.297911 | 0.217151 | 0.249518 | 0 | 12 | 505.6764 | 0.793361 |
| 162 | −0.36751 | 0 | 0.304766 | 0.234822 | 0 | 0.26618 | 12 | 504.1143 | 0.7943 |
| 163 | −0.35702 | 0 | 0.341819 | 0 | 0.21209 | 0.213821 | 12 | 504.4655 | 0.794055 |
| 164 | −0.36116 | 0 | 0 | 0.256551 | 0.172681 | 0.188346 | 12 | 505.2876 | 0.793055 |
| 165 | −0.3784 | −0.48038 | 0.429094 | 0.184036 | 0.235336 | 0 | 12 | 498.112 | 0.799955 |
| 166 | −0.37944 | −0.47686 | 0.432955 | 0.204998 | 0 | 0.247828 | 12 | 498.0143 | 0.800098 |
| 167 | −0.37057 | −0.48826 | 0.469023 | 0 | 0.198485 | 0.194003 | 12 | 497.7535 | 0.800894 |
| 168 | −0.36821 | −0.31823 | 0 | 0.248938 | 0.16128 | 0.17389 | 12 | 504.304 | 0.792422 |
| 169 | −0.36844 | 0 | 0.31303 | 0.214129 | 0.186465 | 0.210249 | 12 | 507.3432 | 0.789564 |
| 170 | −0.37665 | −0.49016 | 0.415897 | 0.213769 | 0 | 0 | 12 | 497.1759 | 0.802834 |
| 171 | −0.3692 | −0.49878 | 0.458935 | 0 | 0.257995 | 0 | 12 | 495.7917 | 0.805345 |
| 172 | −0.36789 | −0.4958 | 0.464972 | 0 | 0 | 0.255069 | 12 | 494.0875 | 0.807019 |
| 173 | −0.36683 | −0.32775 | 0 | 0.247282 | 0.215748 | 0 | 12 | 500.8987 | 0.797158 |
| 174 | −0.36769 | −0.32552 | 0 | 0.266766 | 0 | 0.223296 | 12 | 498.932 | 0.800751 |
| 175 | −0.35346 | −0.32601 | 0 | 0 | 0.190682 | 0.171912 | 12 | 500.6699 | 0.797873 |
| 176 | −0.36573 | 0 | 0.297911 | 0.217151 | 0.249518 | 0 | 12 | 506.3576 | 0.791115 |
| 177 | −0.36751 | 0 | 0.304766 | 0.234822 | 0 | 0.26618 | 12 | 504.2626 | 0.794137 |
| 178 | −0.35702 | 0 | 0.341819 | 0 | 0.21209 | 0.213821 | 12 | 504.9829 | 0.7933 |
| 179 | −0.36116 | 0 | 0 | 0.256551 | 0.172681 | 0.188346 | 12 | 505.592 | 0.792973 |
| 180 | −0.3784 | −0.48038 | 0.429094 | 0.184036 | 0.235336 | 0 | 12 | 500.0917 | 0.79722 |
| 181 | −0.37944 | −0.47686 | 0.432955 | 0.204998 | 0 | 0.247828 | 12 | 499.287 | 0.799486 |
| 182 | −0.37057 | −0.48826 | 0.469023 | 0 | 0.198485 | 0.194003 | 12 | 499.5905 | 0.798036 |
| 183 | −0.36821 | −0.31823 | 0 | 0.248938 | 0.16128 | 0.17389 | 12 | 505.6387 | 0.790544 |
| 184 | −0.36844 | 0 | 0.31303 | 0.214129 | 0.186465 | 0.210249 | 12 | 508.855 | 0.78738 |
| 185 | −0.3784 | −0.48038 | 0.429094 | 0.184036 | 0.235336 | 0 | 12 | 494.6845 | 0.805243 |
| 186 | −0.37944 | −0.47686 | 0.432955 | 0.204998 | 0 | 0.247828 | 12 | 492.6199 | 0.808162 |
| 187 | −0.37057 | −0.48826 | 0.469023 | 0 | 0.198485 | 0.194003 | 12 | 493.0132 | 0.807059 |
| 188 | −0.36821 | −0.31823 | 0 | 0.248938 | 0.16128 | 0.17389 | 12 | 498.4242 | 0.800078 |
| 189 | −0.36844 | 0 | 0.31303 | 0.214129 | 0.186465 | 0.210249 | 12 | 503.492 | 0.795015 |
| 190 | −0.38017 | −0.47149 | 0.43946 | 0.184707 | 0.175726 | 0.194494 | 12 | 498.667 | 0.799628 |
| 191 | −0.37665 | −0.49016 | 0.415897 | 0.213769 | 0 | 0 | 12 | 496.5188 | 0.80316 |
| 192 | −0.3692 | −0.49878 | 0.458935 | 0 | 0.257995 | 0 | 12 | 494.6301 | 0.806488 |
| 193 | −0.36789 | −0.4958 | 0.464972 | 0 | 0 | 0.255069 | 12 | 493.4489 | 0.806917 |
| 194 | −0.36683 | −0.32775 | 0 | 0.247282 | 0.215748 | 0 | 12 | 499.9705 | 0.798097 |
| 195 | −0.36769 | −0.32552 | 0 | 0.266766 | 0 | 0.223296 | 12 | 498.3662 | 0.800833 |
| 196 | −0.35346 | −0.32601 | 0 | 0 | 0.190682 | 0.171912 | 12 | 499.8703 | 0.799281 |
| 197 | −0.36573 | 0 | 0.297911 | 0.217151 | 0.249518 | 0 | 12 | 505.1748 | 0.793892 |
| 198 | −0.36751 | 0 | 0.304766 | 0.234822 | 0 | 0.26618 | 12 | 503.5176 | 0.795443 |
| 199 | −0.35702 | 0 | 0.341819 | 0 | 0.21209 | 0.213821 | 12 | 503.895 | 0.795586 |
| 200 | −0.36116 | 0 | 0 | 0.256551 | 0.172681 | 0.188346 | 12 | 504.6515 | 0.793892 |
| 201 | −0.3784 | −0.48038 | 0.429094 | 0.184036 | 0.235336 | 0 | 12 | 497.7361 | 0.799771 |
| 202 | −0.37944 | −0.47686 | 0.432955 | 0.204998 | 0 | 0.247828 | 12 | 497.5803 | 0.800853 |
| 203 | −0.37057 | −0.48826 | 0.469023 | 0 | 0.198485 | 0.194003 | 12 | 497.3254 | 0.80069 |
| 204 | −0.36821 | −0.31823 | 0 | 0.248938 | 0.16128 | 0.17389 | 12 | 503.7197 | 0.793647 |
| 205 | −0.36844 | 0 | 0.31303 | 0.214129 | 0.186465 | 0.210249 | 12 | 506.7676 | 0.791218 |
| 206 | −0.3784 | −0.48038 | 0.429094 | 0.184036 | 0.235336 | 0 | 12 | 493.3335 | 0.805998 |
| 207 | −0.37944 | −0.47686 | 0.432955 | 0.204998 | 0 | 0.247828 | 12 | 491.7713 | 0.808284 |
| 208 | −0.37057 | −0.48826 | 0.469023 | 0 | 0.198485 | 0.194003 | 12 | 491.7851 | 0.808448 |
| 209 | −0.36821 | −0.31823 | 0 | 0.248938 | 0.16128 | 0.17389 | 12 | 497.44 | 0.801343 |
| 210 | −0.36844 | 0 | 0.31303 | 0.214129 | 0.186465 | 0.210249 | 12 | 502.2744 | 0.796362 |
| 211 | −0.38017 | −0.47149 | 0.43946 | 0.184707 | 0.175726 | 0.194494 | 12 | 496.2633 | 0.801466 |
| 212 | −0.3784 | −0.48038 | 0.429094 | 0.184036 | 0.235336 | 0 | 12 | 494.3068 | 0.805569 |
| 213 | −0.37944 | −0.47686 | 0.432955 | 0.204998 | 0 | 0.247828 | 12 | 492.1825 | 0.808203 |
| 214 | −0.37057 | −0.48826 | 0.469023 | 0 | 0.198485 | 0.194003 | 12 | 492.5817 | 0.807856 |
| 215 | −0.36821 | −0.31823 | 0 | 0.248938 | 0.16128 | 0.17389 | 12 | 497.8612 | 0.801588 |
| 216 | −0.36844 | 0 | 0.31303 | 0.214129 | 0.186465 | 0.210249 | 12 | 502.851 | 0.795198 |
| 217 | −0.38017 | −0.47149 | 0.43946 | 0.184707 | 0.175726 | 0.194494 | 12 | 498.3187 | 0.799833 |
| 218 | −0.38017 | −0.47149 | 0.43946 | 0.184707 | 0.175726 | 0.194494 | 12 | 491.3764 | 0.808223 |
| 219 | −0.37665 | −0.49016 | 0.415897 | 0.213769 | 0 | 0 | 12 | 500.78 | 0.798914 |
| 220 | −0.3692 | −0.49878 | 0.458935 | 0 | 0.257995 | 0 | 12 | 499.4375 | 0.799241 |
| 221 | −0.36789 | −0.4958 | 0.464972 | 0 | 0 | 0.255069 | 12 | 498.2171 | 0.801037 |
| 222 | −0.36683 | −0.32775 | 0 | 0.247282 | 0.215748 | 0 | 12 | 503.7373 | 0.792647 |
| 223 | −0.36769 | −0.32552 | 0 | 0.266766 | 0 | 0.223296 | 12 | 502.1334 | 0.795076 |
| 224 | −0.35346 | −0.32601 | 0 | 0 | 0.190682 | 0.171912 | 12 | 503.8893 | 0.793626 |
| 225 | −0.36573 | 0 | 0.297911 | 0.217151 | 0.249518 | 0 | 12 | 509.0619 | 0.788584 |
| 226 | −0.36751 | 0 | 0.304766 | 0.234822 | 0 | 0.26618 | 12 | 507.3636 | 0.790523 |
| 227 | −0.35702 | 0 | 0.341819 | 0 | 0.21209 | 0.213821 | 12 | 508.1366 | 0.789686 |
| 228 | −0.36116 | 0 | 0 | 0.256551 | 0.172681 | 0.188346 | 12 | 508.2281 | 0.788686 |
| 229 | −0.3784 | −0.48038 | 0.429094 | 0.184036 | 0.235336 | 0 | 12 | 501.8159 | 0.79626 |
| 230 | −0.37944 | −0.47686 | 0.432955 | 0.204998 | 0 | 0.247828 | 12 | 501.4088 | 0.795239 |
| 231 | −0.37057 | −0.48826 | 0.469023 | 0 | 0.198485 | 0.194003 | 12 | 501.5719 | 0.796015 |
| 232 | −0.36821 | −0.31823 | 0 | 0.248938 | 0.16128 | 0.17389 | 12 | 506.8555 | 0.789176 |
| 233 | −0.36844 | 0 | 0.31303 | 0.214129 | 0.186465 | 0.210249 | 12 | 510.2002 | 0.785808 |
| 234 | −0.3784 | −0.48038 | 0.429094 | 0.184036 | 0.235336 | 0 | 12 | 497.8466 | 0.800874 |
| 235 | −0.37944 | −0.47686 | 0.432955 | 0.204998 | 0 | 0.247828 | 12 | 496.2216 | 0.802364 |
| 236 | −0.37057 | −0.48826 | 0.469023 | 0 | 0.198485 | 0.194003 | 12 | 496.6606 | 0.802384 |
| 237 | −0.36821 | −0.31823 | 0 | 0.248938 | 0.16128 | 0.17389 | 12 | 501.1576 | 0.796485 |
| 238 | −0.36844 | 0 | 0.31303 | 0.214129 | 0.186465 | 0.210249 | 12 | 506.1084 | 0.791463 |
| 239 | −0.38017 | −0.47149 | 0.43946 | 0.184707 | 0.175726 | 0.194494 | 12 | 500.0942 | 0.797587 |
| 240 | −0.3784 | −0.48038 | 0.429094 | 0.184036 | 0.235336 | 0 | 12 | 499.3652 | 0.800282 |
| 241 | −0.37944 | −0.47686 | 0.432955 | 0.204998 | 0 | 0.247828 | 12 | 497.121 | 0.801588 |
| 242 | −0.37057 | −0.48826 | 0.469023 | 0 | 0.198485 | 0.194003 | 12 | 498.0134 | 0.800527 |
| 243 | −0.36821 | −0.31823 | 0 | 0.248938 | 0.16128 | 0.17389 | 12 | 502.117 | 0.795464 |
| 244 | −0.36844 | 0 | 0.31303 | 0.214129 | 0.186465 | 0.210249 | 12 | 507.2613 | 0.789339 |
| 245 | −0.38017 | −0.47149 | 0.43946 | 0.184707 | 0.175726 | 0.194494 | 12 | 502.5344 | 0.795198 |
| 246 | −0.38017 | −0.47149 | 0.43946 | 0.184707 | 0.175726 | 0.194494 | 12 | 496.3763 | 0.802446 |
| 247 | −0.3784 | −0.48038 | 0.429094 | 0.184036 | 0.235336 | 0 | 12 | 497.9563 | 0.80071 |
| 248 | −0.37944 | −0.47686 | 0.432955 | 0.204998 | 0 | 0.247828 | 12 | 496.1895 | 0.802119 |
| 249 | −0.37057 | −0.48826 | 0.469023 | 0 | 0.198485 | 0.194003 | 12 | 496.716 | 0.801935 |
| 250 | −0.36821 | −0.31823 | 0 | 0.248938 | 0.16128 | 0.17389 | 12 | 501.0518 | 0.796199 |
| 251 | −0.36844 | 0 | 0.31303 | 0.214129 | 0.186465 | 0.210249 | 12 | 505.9062 | 0.791565 |
| 252 | −0.38017 | −0.47149 | 0.43946 | 0.184707 | 0.175726 | 0.194494 | 12 | 499.9073 | 0.797485 |
| 253 | −0.38017 | −0.47149 | 0.43946 | 0.184707 | 0.175726 | 0.194494 | 12 | 494.9271 | 0.802956 |
| 254 | −0.38017 | −0.47149 | 0.43946 | 0.184707 | 0.175726 | 0.194494 | 12 | 496.2407 | 0.802711 |
| 255 | −0.37665 | −0.49016 | 0.415897 | 0.213769 | 0 | 0 | 12 | 540.8306 | 0.748061 |
| 256 | −0.3692 | −0.49878 | 0.458935 | 0 | 0.257995 | 0 | 12 | 537.8271 | 0.752184 |
| 257 | −0.36789 | −0.4958 | 0.464972 | 0 | 0 | 0.255069 | 12 | 536.6497 | 0.754308 |
| 258 | −0.36683 | −0.32775 | 0 | 0.247282 | 0.215748 | 0 | 12 | 551.276 | 0.734913 |
| 259 | −0.36769 | −0.32552 | 0 | 0.266766 | 0 | 0.223296 | 12 | 549.6328 | 0.736812 |
| 260 | −0.35346 | −0.32601 | 0 | 0 | 0.190682 | 0.171912 | 12 | 551.9638 | 0.73328 |
| 261 | −0.36573 | 0 | 0.297911 | 0.217151 | 0.249518 | 0 | 12 | 544.0853 | 0.744345 |
| 262 | −0.36751 | 0 | 0.304766 | 0.234822 | 0 | 0.26618 | 12 | 541.7777 | 0.748857 |
| 263 | −0.35702 | 0 | 0.341819 | 0 | 0.21209 | 0.213821 | 12 | 542.0654 | 0.747142 |
| 264 | −0.36116 | 0 | 0 | 0.256551 | 0.172681 | 0.188346 | 12 | 550.2015 | 0.736526 |
| 265 | −0.3784 | −0.48038 | 0.429094 | 0.184036 | 0.235336 | 0 | 12 | 538.9286 | 0.750204 |
| 266 | −0.37944 | −0.47686 | 0.432955 | 0.204998 | 0 | 0.247828 | 12 | 538.4815 | 0.750715 |
| 267 | −0.37057 | −0.48826 | 0.469023 | 0 | 0.198485 | 0.194003 | 12 | 537.9002 | 0.750102 |
| 268 | −0.36821 | −0.31823 | 0 | 0.248938 | 0.16128 | 0.17389 | 12 | 552.5488 | 0.730177 |
| 269 | −0.36844 | 0 | 0.31303 | 0.214129 | 0.186465 | 0.210249 | 12 | 543.2977 | 0.744202 |
| 270 | −0.3784 | −0.48038 | 0.429094 | 0.184036 | 0.235336 | 0 | 12 | 536.2751 | 0.753368 |
| 271 | −0.37944 | −0.47686 | 0.432955 | 0.204998 | 0 | 0.247828 | 12 | 534.5219 | 0.755818 |
| 272 | −0.37057 | −0.48826 | 0.469023 | 0 | 0.198485 | 0.194003 | 12 | 534.0873 | 0.755839 |
| 273 | −0.36821 | −0.31823 | 0 | 0.248938 | 0.16128 | 0.17389 | 12 | 548.4167 | 0.735853 |
| 274 | −0.36844 | 0 | 0.31303 | 0.214129 | 0.186465 | 0.210249 | 12 | 539.9083 | 0.749714 |
| 275 | −0.38017 | −0.47149 | 0.43946 | 0.184707 | 0.175726 | 0.194494 | 12 | 536.4224 | 0.752368 |
| 276 | −0.3784 | −0.48038 | 0.429094 | 0.184036 | 0.235336 | 0 | 12 | 537.3122 | 0.75149 |
| 277 | −0.37944 | −0.47686 | 0.432955 | 0.204998 | 0 | 0.247828 | 12 | 534.8751 | 0.755634 |
| 278 | −0.37057 | −0.48826 | 0.469023 | 0 | 0.198485 | 0.194003 | 12 | 535.0134 | 0.75445 |
| 279 | −0.36821 | −0.31823 | 0 | 0.248938 | 0.16128 | 0.17389 | 12 | 548.6553 | 0.736016 |
| 280 | −0.36844 | 0 | 0.31303 | 0.214129 | 0.186465 | 0.210249 | 12 | 540.5737 | 0.749143 |
| 281 | −0.38017 | −0.47149 | 0.43946 | 0.184707 | 0.175726 | 0.194494 | 12 | 538.0868 | 0.750061 |
| 282 | −0.38017 | −0.47149 | 0.43946 | 0.184707 | 0.175726 | 0.194494 | 12 | 533.4026 | 0.75541 |
| 283 | −0.3784 | −0.48038 | 0.429094 | 0.184036 | 0.235336 | 0 | 12 | 536.1863 | 0.755063 |
| 284 | −0.37944 | −0.47686 | 0.432955 | 0.204998 | 0 | 0.247828 | 12 | 534.3006 | 0.757023 |
| 285 | −0.37057 | −0.48826 | 0.469023 | 0 | 0.198485 | 0.194003 | 12 | 533.9915 | 0.756819 |
| 286 | −0.36821 | −0.31823 | 0 | 0.248938 | 0.16128 | 0.17389 | 12 | 547.8999 | 0.738486 |
| 287 | −0.36844 | 0 | 0.31303 | 0.214129 | 0.186465 | 0.210249 | 12 | 539.4624 | 0.75149 |
| 288 | −0.38017 | −0.47149 | 0.43946 | 0.184707 | 0.175726 | 0.194494 | 12 | 536.1693 | 0.753654 |
| 289 | −0.38017 | −0.47149 | 0.43946 | 0.184707 | 0.175726 | 0.194494 | 12 | 532.2482 | 0.758431 |
| 290 | −0.38017 | −0.47149 | 0.43946 | 0.184707 | 0.175726 | 0.194494 | 12 | 533.135 | 0.756553 |
| 291 | −0.3784 | −0.48038 | 0.429094 | 0.184036 | 0.235336 | 0 | 12 | 537.6084 | 0.753123 |
| 292 | −0.37944 | −0.47686 | 0.432955 | 0.204998 | 0 | 0.247828 | 12 | 535.5793 | 0.755063 |
| 293 | −0.37057 | −0.48826 | 0.469023 | 0 | 0.198485 | 0.194003 | 12 | 535.5644 | 0.754614 |
| 294 | −0.36821 | −0.31823 | 0 | 0.248938 | 0.16128 | 0.17389 | 12 | 548.6585 | 0.736485 |
| 295 | −0.36844 | 0 | 0.31303 | 0.214129 | 0.186465 | 0.210249 | 12 | 540.8765 | 0.748877 |
| 296 | −0.38017 | −0.47149 | 0.43946 | 0.184707 | 0.175726 | 0.194494 | 12 | 537.4083 | 0.752572 |
| 297 | −0.38017 | −0.47149 | 0.43946 | 0.184707 | 0.175726 | 0.194494 | 12 | 533.6353 | 0.757002 |
| 298 | −0.38017 | −0.47149 | 0.43946 | 0.184707 | 0.175726 | 0.194494 | 12 | 534.9041 | 0.754328 |
| 299 | −0.38017 | −0.47149 | 0.43946 | 0.184707 | 0.175726 | 0.194494 | 12 | 533.6725 | 0.757574 |
| 300 | −0.34604 | 0 | 0 | 0 | 0.20186 | 0.189915 | 11 | 542.6062 | 0.744161 |
| 301 | −0.36484 | −0.51223 | 0.448932 | 0 | 0 | 0 | 11 | 531.8912 | 0.761004 |
| 302 | −0.36535 | −0.34122 | 0 | 0.272545 | 0 | 0 | 11 | 534.6561 | 0.759636 |
| 303 | −0.35247 | −0.33749 | 0 | 0 | 0.244058 | 0 | 11 | 532.8627 | 0.759411 |
| 304 | −0.3509 | −0.33426 | 0 | 0 | 0 | 0.230316 | 11 | 532.7881 | 0.761024 |
| 305 | −0.3635 | 0 | 0.280897 | 0.247949 | 0 | 0 | 11 | 548.2008 | 0.734995 |
| 306 | −0.35435 | 0 | 0.326983 | 0 | 0.276248 | 0 | 11 | 544.6255 | 0.740936 |
| 307 | −0.35429 | 0 | 0.335115 | 0 | 0 | 0.278056 | 11 | 544.5923 | 0.741017 |
| 308 | −0.35918 | 0 | 0 | 0.257296 | 0.231108 | 0 | 11 | 542.8526 | 0.745631 |
| 309 | −0.36046 | 0 | 0 | 0.27483 | 0 | 0.241127 | 11 | 541.9474 | 0.747918 |
| 310 | −0.37665 | −0.49016 | 0.415897 | 0.213769 | 0 | 0 | 11 | 533.548 | 0.757574 |
| 311 | −0.3692 | −0.49878 | 0.458935 | 0 | 0.257995 | 0 | 11 | 529.9753 | 0.760922 |
| 312 | −0.36789 | −0.4958 | 0.464972 | 0 | 0 | 0.255069 | 11 | 532.0698 | 0.755594 |
| 313 | −0.36683 | −0.32775 | 0 | 0.247282 | 0.215748 | 0 | 11 | 535.3525 | 0.756267 |
| 314 | −0.36769 | −0.32552 | 0 | 0.266766 | 0 | 0.223296 | 11 | 536.4128 | 0.756431 |
| 315 | −0.35346 | −0.32601 | 0 | 0 | 0.190682 | 0.171912 | 11 | 537.0207 | 0.752001 |
| 316 | −0.36573 | 0 | 0.297911 | 0.217151 | 0.249518 | 0 | 11 | 545.8808 | 0.738486 |
| 317 | −0.36751 | 0 | 0.304766 | 0.234822 | 0 | 0.26618 | 11 | 546.919 | 0.739588 |
| 318 | −0.35702 | 0 | 0.341819 | 0 | 0.21209 | 0.213821 | 11 | 546.0781 | 0.738935 |
| 319 | −0.36116 | 0 | 0 | 0.256551 | 0.172681 | 0.188346 | 11 | 545.4007 | 0.740487 |
| 320 | −0.37665 | −0.49016 | 0.415897 | 0.213769 | 0 | 0 | 11 | 529.5715 | 0.76478 |
| 321 | −0.3692 | −0.49878 | 0.458935 | 0 | 0.257995 | 0 | 11 | 525.6336 | 0.768414 |
| 322 | −0.36789 | −0.4958 | 0.464972 | 0 | 0 | 0.255069 | 11 | 525.7912 | 0.766903 |
| 323 | −0.36683 | −0.32775 | 0 | 0.247282 | 0.215748 | 0 | 11 | 530.1121 | 0.764331 |
| 324 | −0.36769 | −0.32552 | 0 | 0.266766 | 0 | 0.223296 | 11 | 529.2396 | 0.766311 |
| 325 | −0.35346 | −0.32601 | 0 | 0 | 0.190682 | 0.171912 | 11 | 530.2442 | 0.762535 |
| 326 | −0.36573 | 0 | 0.297911 | 0.217151 | 0.249518 | 0 | 11 | 542.6377 | 0.743978 |
| 327 | −0.36751 | 0 | 0.304766 | 0.234822 | 0 | 0.26618 | 11 | 541.9205 | 0.746264 |
| 328 | −0.35702 | 0 | 0.341819 | 0 | 0.21209 | 0.213821 | 11 | 541.3213 | 0.745897 |
| 329 | −0.36116 | 0 | 0 | 0.256551 | 0.172681 | 0.188346 | 11 | 539.773 | 0.750245 |
| 330 | −0.3784 | −0.48038 | 0.429094 | 0.184036 | 0.235336 | 0 | 11 | 528.5968 | 0.763698 |
| 331 | −0.37944 | −0.47686 | 0.432955 | 0.204998 | 0 | 0.247828 | 11 | 530.0687 | 0.760942 |
| 332 | −0.37057 | −0.48826 | 0.469023 | 0 | 0.198485 | 0.194003 | 11 | 528.7241 | 0.761514 |
| 333 | −0.36821 | −0.31823 | 0 | 0.248938 | 0.16128 | 0.17389 | 11 | 534.4352 | 0.758207 |
| 334 | −0.36844 | 0 | 0.31303 | 0.214129 | 0.186465 | 0.210249 | 11 | 544.2356 | 0.742487 |
| 335 | −0.37665 | −0.49016 | 0.415897 | 0.213769 | 0 | 0 | 11 | 529.2822 | 0.764801 |
| 336 | −0.3692 | −0.49878 | 0.458935 | 0 | 0.257995 | 0 | 11 | 525.895 | 0.767843 |
| 337 | −0.36789 | −0.4958 | 0.464972 | 0 | 0 | 0.255069 | 11 | 525.3589 | 0.767945 |
| 338 | −0.36683 | −0.32775 | 0 | 0.247282 | 0.215748 | 0 | 11 | 530.0756 | 0.763923 |
| 339 | −0.36769 | −0.32552 | 0 | 0.266766 | 0 | 0.223296 | 11 | 528.669 | 0.768741 |
| 340 | −0.35346 | −0.32601 | 0 | 0 | 0.190682 | 0.171912 | 11 | 529.9219 | 0.762576 |
| 341 | −0.36573 | 0 | 0.297911 | 0.217151 | 0.249518 | 0 | 11 | 542.504 | 0.743181 |
| 342 | −0.36751 | 0 | 0.304766 | 0.234822 | 0 | 0.26618 | 11 | 541.2526 | 0.747142 |
| 343 | −0.35702 | 0 | 0.341819 | 0 | 0.21209 | 0.213821 | 11 | 540.9312 | 0.745713 |
| 344 | −0.36116 | 0 | 0 | 0.256551 | 0.172681 | 0.188346 | 11 | 539.3523 | 0.748938 |
| 345 | −0.3784 | −0.48038 | 0.429094 | 0.184036 | 0.235336 | 0 | 11 | 529.7688 | 0.761596 |
| 346 | −0.37944 | −0.47686 | 0.432955 | 0.204998 | 0 | 0.247828 | 11 | 530.4673 | 0.761575 |
| 347 | −0.37057 | −0.48826 | 0.469023 | 0 | 0.198485 | 0.194003 | 11 | 529.6937 | 0.760779 |
| 348 | −0.36821 | −0.31823 | 0 | 0.248938 | 0.16128 | 0.17389 | 11 | 535.0318 | 0.756778 |
| 349 | −0.36844 | 0 | 0.31303 | 0.214129 | 0.186465 | 0.210249 | 11 | 544.7628 | 0.740242 |
| 350 | −0.3784 | −0.48038 | 0.429094 | 0.184036 | 0.235336 | 0 | 11 | 524.4881 | 0.770211 |
| 351 | −0.37944 | −0.47686 | 0.432955 | 0.204998 | 0 | 0.247828 | 11 | 523.3022 | 0.77213 |
| 352 | −0.37057 | −0.48826 | 0.469023 | 0 | 0.198485 | 0.194003 | 11 | 522.6728 | 0.770231 |
| 353 | −0.36821 | −0.31823 | 0 | 0.248938 | 0.16128 | 0.17389 | 11 | 527.1022 | 0.768394 |
| 354 | −0.36844 | 0 | 0.31303 | 0.214129 | 0.186465 | 0.210249 | 11 | 539.0379 | 0.748285 |
| 355 | −0.38017 | −0.47149 | 0.43946 | 0.184707 | 0.175726 | 0.194494 | 11 | 528.4292 | 0.763637 |
| 356 | −0.37665 | −0.49016 | 0.415897 | 0.213769 | 0 | 0 | 11 | 529.9187 | 0.763433 |
| 357 | −0.3692 | −0.49878 | 0.458935 | 0 | 0.257995 | 0 | 11 | 526.2023 | 0.765821 |
| 358 | −0.36789 | −0.4958 | 0.464972 | 0 | 0 | 0.255069 | 11 | 526.0376 | 0.766352 |
| 359 | −0.36683 | −0.32775 | 0 | 0.247282 | 0.215748 | 0 | 11 | 530.2227 | 0.763004 |
| 360 | −0.36769 | −0.32552 | 0 | 0.266766 | 0 | 0.223296 | 11 | 529.0409 | 0.767822 |
| 361 | −0.35346 | −0.32601 | 0 | 0 | 0.190682 | 0.171912 | 11 | 530.3073 | 0.760983 |
| 362 | −0.36573 | 0 | 0.297911 | 0.217151 | 0.249518 | 0 | 11 | 542.6251 | 0.742936 |
| 363 | −0.36751 | 0 | 0.304766 | 0.234822 | 0 | 0.26618 | 11 | 541.6101 | 0.746979 |
| 364 | −0.35702 | 0 | 0.341819 | 0 | 0.21209 | 0.213821 | 11 | 541.2064 | 0.745692 |
| 365 | −0.36116 | 0 | 0 | 0.256551 | 0.172681 | 0.188346 | 11 | 539.4339 | 0.749959 |
| 366 | −0.3784 | −0.48038 | 0.429094 | 0.184036 | 0.235336 | 0 | 11 | 528.5176 | 0.762351 |
| 367 | −0.37944 | −0.47686 | 0.432955 | 0.204998 | 0 | 0.247828 | 11 | 529.6871 | 0.762106 |
| 368 | −0.37057 | −0.48826 | 0.469023 | 0 | 0.198485 | 0.194003 | 11 | 528.4781 | 0.760759 |
| 369 | −0.36821 | −0.31823 | 0 | 0.248938 | 0.16128 | 0.17389 | 11 | 533.7854 | 0.757819 |
| 370 | −0.36844 | 0 | 0.31303 | 0.214129 | 0.186465 | 0.210249 | 11 | 543.5444 | 0.742855 |
| 371 | −0.3784 | −0.48038 | 0.429094 | 0.184036 | 0.235336 | 0 | 11 | 524.3364 | 0.770129 |
| 372 | −0.37944 | −0.47686 | 0.432955 | 0.204998 | 0 | 0.247828 | 11 | 523.4726 | 0.772089 |
| 373 | −0.37057 | −0.48826 | 0.469023 | 0 | 0.198485 | 0.194003 | 11 | 522.6941 | 0.769904 |
| 374 | −0.36821 | −0.31823 | 0 | 0.248938 | 0.16128 | 0.17389 | 11 | 526.9774 | 0.769231 |
| 375 | −0.36844 | 0 | 0.31303 | 0.214129 | 0.186465 | 0.210249 | 11 | 538.8484 | 0.74953 |
| 376 | −0.38017 | −0.47149 | 0.43946 | 0.184707 | 0.175726 | 0.194494 | 11 | 526.8635 | 0.763923 |
| 377 | −0.3784 | −0.48038 | 0.429094 | 0.184036 | 0.235336 | 0 | 11 | 524.5723 | 0.769292 |
| 378 | −0.37944 | −0.47686 | 0.432955 | 0.204998 | 0 | 0.247828 | 11 | 523.2078 | 0.771538 |
| 379 | −0.37057 | −0.48826 | 0.469023 | 0 | 0.198485 | 0.194003 | 11 | 522.7056 | 0.769537 |
| 380 | −0.36821 | −0.31823 | 0 | 0.248938 | 0.16128 | 0.17389 | 11 | 526.7744 | 0.768475 |
| 381 | −0.36844 | 0 | 0.31303 | 0.214129 | 0.186465 | 0.210249 | 11 | 538.57 | 0.750245 |
| 382 | −0.38017 | −0.47149 | 0.43946 | 0.184707 | 0.175726 | 0.194494 | 11 | 527.8511 | 0.763127 |
| 383 | −0.38017 | −0.47149 | 0.43946 | 0.184707 | 0.175726 | 0.194494 | 11 | 520.892 | 0.773416 |
| 384 | −0.37665 | −0.49016 | 0.415897 | 0.213769 | 0 | 0 | 11 | 534.8195 | 0.756533 |
| 385 | −0.3692 | −0.49878 | 0.458935 | 0 | 0.257995 | 0 | 11 | 531.8894 | 0.757431 |
| 386 | −0.36789 | −0.4958 | 0.464972 | 0 | 0 | 0.255069 | 11 | 531.5788 | 0.758533 |
| 387 | −0.36683 | −0.32775 | 0 | 0.247282 | 0.215748 | 0 | 11 | 534.7311 | 0.757696 |
| 388 | −0.36769 | −0.32552 | 0 | 0.266766 | 0 | 0.223296 | 11 | 533.5185 | 0.760371 |
| 389 | −0.35346 | −0.32601 | 0 | 0 | 0.190682 | 0.171912 | 11 | 535.1465 | 0.755369 |
| 390 | −0.36573 | 0 | 0.297911 | 0.217151 | 0.249518 | 0 | 11 | 547.3015 | 0.733934 |
| 391 | −0.36751 | 0 | 0.304766 | 0.234822 | 0 | 0.26618 | 11 | 546.1824 | 0.737669 |
| 392 | −0.35702 | 0 | 0.341819 | 0 | 0.21209 | 0.213821 | 11 | 546.3647 | 0.734423 |
| 393 | −0.36116 | 0 | 0 | 0.256551 | 0.172681 | 0.188346 | 11 | 543.7543 | 0.740977 |
| 394 | −0.3784 | −0.48038 | 0.429094 | 0.184036 | 0.235336 | 0 | 11 | 533.7875 | 0.755879 |
| 395 | −0.37944 | −0.47686 | 0.432955 | 0.204998 | 0 | 0.247828 | 11 | 534.5892 | 0.754797 |
| 396 | −0.37057 | −0.48826 | 0.469023 | 0 | 0.198485 | 0.194003 | 11 | 534.0234 | 0.754022 |
| 397 | −0.36821 | −0.31823 | 0 | 0.248938 | 0.16128 | 0.17389 | 11 | 538.013 | 0.752429 |
| 398 | −0.36844 | 0 | 0.31303 | 0.214129 | 0.186465 | 0.210249 | 11 | 548.2757 | 0.733586 |
| 399 | −0.3784 | −0.48038 | 0.429094 | 0.184036 | 0.235336 | 0 | 11 | 529.6326 | 0.761922 |
| 400 | −0.37944 | −0.47686 | 0.432955 | 0.204998 | 0 | 0.247828 | 11 | 528.6128 | 0.76525 |
| 401 | −0.37057 | −0.48826 | 0.469023 | 0 | 0.198485 | 0.194003 | 11 | 528.4114 | 0.761453 |
| 402 | −0.36821 | −0.31823 | 0 | 0.248938 | 0.16128 | 0.17389 | 11 | 531.4097 | 0.762228 |
| 403 | −0.36844 | 0 | 0.31303 | 0.214129 | 0.186465 | 0.210249 | 11 | 543.448 | 0.740466 |
| 404 | −0.38017 | −0.47149 | 0.43946 | 0.184707 | 0.175726 | 0.194494 | 11 | 531.8694 | 0.758431 |
| 405 | −0.3784 | −0.48038 | 0.429094 | 0.184036 | 0.235336 | 0 | 11 | 530.6101 | 0.760922 |
| 406 | −0.37944 | −0.47686 | 0.432955 | 0.204998 | 0 | 0.247828 | 11 | 528.958 | 0.765658 |
| 407 | −0.37057 | −0.48826 | 0.469023 | 0 | 0.198485 | 0.194003 | 11 | 529.1943 | 0.76082 |
| 408 | −0.36821 | −0.31823 | 0 | 0.248938 | 0.16128 | 0.17389 | 11 | 531.9489 | 0.760595 |
| 409 | −0.36844 | 0 | 0.31303 | 0.214129 | 0.186465 | 0.210249 | 11 | 543.9556 | 0.738241 |
| 410 | −0.38017 | −0.47149 | 0.43946 | 0.184707 | 0.175726 | 0.194494 | 11 | 533.6124 | 0.754695 |
| 411 | −0.38017 | −0.47149 | 0.43946 | 0.184707 | 0.175726 | 0.194494 | 11 | 526.8753 | 0.765781 |
| 412 | −0.3784 | −0.48038 | 0.429094 | 0.184036 | 0.235336 | 0 | 11 | 530.7832 | 0.75937 |
| 413 | −0.37944 | −0.47686 | 0.432955 | 0.204998 | 0 | 0.247828 | 11 | 529.4531 | 0.763433 |
| 414 | −0.37057 | −0.48826 | 0.469023 | 0 | 0.198485 | 0.194003 | 11 | 529.5313 | 0.759901 |
| 415 | −0.36821 | −0.31823 | 0 | 0.248938 | 0.16128 | 0.17389 | 11 | 532.102 | 0.760922 |
| 416 | −0.36844 | 0 | 0.31303 | 0.214129 | 0.186465 | 0.210249 | 11 | 544.0568 | 0.73916 |
| 417 | −0.38017 | −0.47149 | 0.43946 | 0.184707 | 0.175726 | 0.194494 | 11 | 532.1663 | 0.757513 |
| 418 | −0.38017 | −0.47149 | 0.43946 | 0.184707 | 0.175726 | 0.194494 | 11 | 526.8265 | 0.765842 |
| 419 | −0.38017 | −0.47149 | 0.43946 | 0.184707 | 0.175726 | 0.194494 | 11 | 527.4548 | 0.764576 |
| 420 | −0.37665 | −0.49016 | 0.415897 | 0.213769 | 0 | 0 | 11 | 562.6302 | 0.707986 |
| 421 | −0.3692 | −0.49878 | 0.458935 | 0 | 0.257995 | 0 | 11 | 558.4106 | 0.714703 |
| 422 | −0.36789 | −0.4958 | 0.464972 | 0 | 0 | 0.255069 | 11 | 558.0278 | 0.713662 |
| 423 | −0.36683 | −0.32775 | 0 | 0.247282 | 0.215748 | 0 | 11 | 570.9054 | 0.697595 |
| 424 | −0.36769 | −0.32552 | 0 | 0.266766 | 0 | 0.223296 | 11 | 569.7095 | 0.696962 |
| 425 | −0.35346 | −0.32601 | 0 | 0 | 0.190682 | 0.171912 | 11 | 572.0795 | 0.694125 |
| 426 | −0.36573 | 0 | 0.297911 | 0.217151 | 0.249518 | 0 | 11 | 570.6337 | 0.69486 |
| 427 | −0.36751 | 0 | 0.304766 | 0.234822 | 0 | 0.26618 | 11 | 569.1028 | 0.697616 |
| 428 | −0.35702 | 0 | 0.341819 | 0 | 0.21209 | 0.213821 | 11 | 568.7843 | 0.697085 |
| 429 | −0.36116 | 0 | 0 | 0.256551 | 0.172681 | 0.188346 | 11 | 574.0129 | 0.689429 |
| 430 | −0.3784 | −0.48038 | 0.429094 | 0.184036 | 0.235336 | 0 | 11 | 559.4847 | 0.711334 |
| 431 | −0.37944 | −0.47686 | 0.432955 | 0.204998 | 0 | 0.247828 | 11 | 560.1887 | 0.709599 |
| 432 | −0.37057 | −0.48826 | 0.469023 | 0 | 0.198485 | 0.194003 | 11 | 559.0553 | 0.710538 |
| 433 | −0.36821 | −0.31823 | 0 | 0.248938 | 0.16128 | 0.17389 | 11 | 572.8037 | 0.689327 |
| 434 | −0.36844 | 0 | 0.31303 | 0.214129 | 0.186465 | 0.210249 | 11 | 570.1958 | 0.69586 |
| 435 | −0.3784 | −0.48038 | 0.429094 | 0.184036 | 0.235336 | 0 | 11 | 556.3832 | 0.716683 |
| 436 | −0.37944 | −0.47686 | 0.432955 | 0.204998 | 0 | 0.247828 | 11 | 555.2423 | 0.716254 |
| 437 | −0.37057 | −0.48826 | 0.469023 | 0 | 0.198485 | 0.194003 | 11 | 554.29 | 0.717418 |
| 438 | −0.36821 | −0.31823 | 0 | 0.248938 | 0.16128 | 0.17389 | 11 | 567.6678 | 0.698269 |
| 439 | −0.36844 | 0 | 0.31303 | 0.214129 | 0.186465 | 0.210249 | 11 | 565.9977 | 0.701209 |
| 440 | −0.38017 | −0.47149 | 0.43946 | 0.184707 | 0.175726 | 0.194494 | 11 | 557.1319 | 0.713049 |
| 441 | −0.3784 | −0.48038 | 0.429094 | 0.184036 | 0.235336 | 0 | 11 | 556.9663 | 0.716397 |
| 442 | −0.37944 | −0.47686 | 0.432955 | 0.204998 | 0 | 0.247828 | 11 | 555.1364 | 0.717112 |
| 443 | −0.37057 | −0.48826 | 0.469023 | 0 | 0.198485 | 0.194003 | 11 | 554.7167 | 0.716948 |
| 444 | −0.36821 | −0.31823 | 0 | 0.248938 | 0.16128 | 0.17389 | 11 | 567.5102 | 0.700188 |
| 445 | −0.36844 | 0 | 0.31303 | 0.214129 | 0.186465 | 0.210249 | 11 | 566.0387 | 0.700923 |
| 446 | −0.38017 | −0.47149 | 0.43946 | 0.184707 | 0.175726 | 0.194494 | 11 | 558.2383 | 0.708272 |
| 447 | −0.38017 | −0.47149 | 0.43946 | 0.184707 | 0.175726 | 0.194494 | 11 | 552.691 | 0.717745 |
| 448 | −0.3784 | −0.48038 | 0.429094 | 0.184036 | 0.235336 | 0 | 11 | 556.8298 | 0.716887 |
| 449 | −0.37944 | −0.47686 | 0.432955 | 0.204998 | 0 | 0.247828 | 11 | 555.4379 | 0.71703 |
| 450 | −0.37057 | −0.48826 | 0.469023 | 0 | 0.198485 | 0.194003 | 11 | 554.7275 | 0.716744 |
| 451 | −0.36821 | −0.31823 | 0 | 0.248938 | 0.16128 | 0.17389 | 11 | 567.4417 | 0.701311 |
| 452 | −0.36844 | 0 | 0.31303 | 0.214129 | 0.186465 | 0.210249 | 11 | 565.7659 | 0.70274 |
| 453 | −0.38017 | −0.47149 | 0.43946 | 0.184707 | 0.175726 | 0.194494 | 11 | 556.8865 | 0.713743 |
| 454 | −0.38017 | −0.47149 | 0.43946 | 0.184707 | 0.175726 | 0.194494 | 11 | 552.3341 | 0.719072 |
| 455 | −0.38017 | −0.47149 | 0.43946 | 0.184707 | 0.175726 | 0.194494 | 11 | 552.6411 | 0.71848 |
| 456 | −0.3784 | −0.48038 | 0.429094 | 0.184036 | 0.235336 | 0 | 11 | 558.6233 | 0.717602 |
| 457 | −0.37944 | −0.47686 | 0.432955 | 0.204998 | 0 | 0.247828 | 11 | 557.0974 | 0.717642 |
| 458 | −0.37057 | −0.48826 | 0.469023 | 0 | 0.198485 | 0.194003 | 11 | 556.7534 | 0.716173 |
| 459 | −0.36821 | −0.31823 | 0 | 0.248938 | 0.16128 | 0.17389 | 11 | 568.4436 | 0.701841 |
| 460 | −0.36844 | 0 | 0.31303 | 0.214129 | 0.186465 | 0.210249 | 11 | 567.7867 | 0.698146 |
| 461 | −0.38017 | −0.47149 | 0.43946 | 0.184707 | 0.175726 | 0.194494 | 11 | 558.9217 | 0.711763 |
| 462 | −0.38017 | −0.47149 | 0.43946 | 0.184707 | 0.175726 | 0.194494 | 11 | 554.1872 | 0.720541 |
| 463 | −0.38017 | −0.47149 | 0.43946 | 0.184707 | 0.175726 | 0.194494 | 11 | 555.0975 | 0.718255 |
| 464 | −0.34954 | 0 | 0.311734 | 0 | 0 | 0 | 11 | 509.1141 | 0.788543 |
| 465 | −0.34838 | −0.35299 | 0 | 0 | 0 | 0 | 11 | 503.8896 | 0.793137 |
| 466 | −0.35731 | 0 | 0 | 0.283735 | 0 | 0 | 11 | 508.6533 | 0.786338 |
| 467 | −0.34421 | 0 | 0 | 0 | 0.260301 | 0 | 11 | 508.1679 | 0.788298 |
| 468 | −0.34349 | 0 | 0 | 0 | 0 | 0.25144 | 11 | 507.2185 | 0.79085 |
| 469 | −0.34604 | 0 | 0 | 0 | 0.20186 | 0.189915 | 11 | 510.662 | 0.783542 |
| 470 | −0.36484 | −0.51223 | 0.448932 | 0 | 0 | 0 | 11 | 500.4309 | 0.79477 |
| 471 | −0.36535 | −0.34122 | 0 | 0.272545 | 0 | 0 | 11 | 505.7332 | 0.788278 |
| 472 | −0.35247 | −0.33749 | 0 | 0 | 0.244058 | 0 | 11 | 505.5545 | 0.788298 |
| 473 | −0.3635 | 0 | 0.280897 | 0.247949 | 0 | 0 | 11 | 510.0417 | 0.784501 |
| 474 | −0.3509 | −0.33426 | 0 | 0 | 0 | 0.230316 | 11 | 505.8684 | 0.789299 |
| 475 | −0.35435 | 0 | 0.326983 | 0 | 0.276248 | 0 | 11 | 508.6725 | 0.786053 |
| 476 | −0.35429 | 0 | 0.335115 | 0 | 0 | 0.278056 | 11 | 508.6482 | 0.786747 |
| 477 | −0.35918 | 0 | 0 | 0.257296 | 0.231108 | 0 | 11 | 509.8964 | 0.783501 |
| 478 | −0.36046 | 0 | 0 | 0.27483 | 0 | 0.241127 | 11 | 509.5526 | 0.784705 |
| 479 | −0.34604 | 0 | 0 | 0 | 0.20186 | 0.189915 | 11 | 505.9325 | 0.78985 |
| 480 | −0.36484 | −0.51223 | 0.448932 | 0 | 0 | 0 | 11 | 496.5616 | 0.801997 |
| 481 | −0.36535 | −0.34122 | 0 | 0.272545 | 0 | 0 | 11 | 501.1472 | 0.795158 |
| 482 | −0.35247 | −0.33749 | 0 | 0 | 0.244058 | 0 | 11 | 500.7712 | 0.795811 |
| 483 | −0.3635 | 0 | 0.280897 | 0.247949 | 0 | 0 | 11 | 507.0393 | 0.789013 |
| 484 | −0.3509 | −0.33426 | 0 | 0 | 0 | 0.230316 | 11 | 499.8758 | 0.796913 |
| 485 | −0.35435 | 0 | 0.326983 | 0 | 0.276248 | 0 | 11 | 505.5017 | 0.792034 |
| 486 | −0.35429 | 0 | 0.335115 | 0 | 0 | 0.278056 | 11 | 504.3925 | 0.792361 |
| 487 | −0.35918 | 0 | 0 | 0.257296 | 0.231108 | 0 | 11 | 506.0199 | 0.789217 |
| 488 | −0.36046 | 0 | 0 | 0.27483 | 0 | 0.241127 | 11 | 504.5835 | 0.791422 |
| 489 | −0.37665 | −0.49016 | 0.415897 | 0.213769 | 0 | 0 | 11 | 498.9393 | 0.796219 |
| 490 | −0.3692 | −0.49878 | 0.458935 | 0 | 0.257995 | 0 | 11 | 497.2243 | 0.798546 |
| 491 | −0.36789 | −0.4958 | 0.464972 | 0 | 0 | 0.255069 | 11 | 497.5485 | 0.798832 |
| 492 | −0.36683 | −0.32775 | 0 | 0.247282 | 0.215748 | 0 | 11 | 503.6127 | 0.789727 |
| 493 | −0.36769 | −0.32552 | 0 | 0.266766 | 0 | 0.223296 | 11 | 503.4787 | 0.790748 |
| 494 | −0.35346 | −0.32601 | 0 | 0 | 0.190682 | 0.171912 | 11 | 504.626 | 0.789768 |
| 495 | −0.36573 | 0 | 0.297911 | 0.217151 | 0.249518 | 0 | 11 | 507.2607 | 0.78742 |
| 496 | −0.36751 | 0 | 0.304766 | 0.234822 | 0 | 0.26618 | 11 | 506.8884 | 0.788135 |
| 497 | −0.35702 | 0 | 0.341819 | 0 | 0.21209 | 0.213821 | 11 | 507.0398 | 0.787972 |
| 498 | −0.36116 | 0 | 0 | 0.256551 | 0.172681 | 0.188346 | 11 | 508.6592 | 0.785461 |
| 499 | −0.34604 | 0 | 0 | 0 | 0.20186 | 0.189915 | 11 | 506.1394 | 0.789462 |
| 500 | −0.36484 | −0.51223 | 0.448932 | 0 | 0 | 0 | 11 | 496.842 | 0.802099 |
| 501 | −0.36535 | −0.34122 | 0 | 0.272545 | 0 | 0 | 11 | 501.1883 | 0.795321 |
| 502 | −0.35247 | −0.33749 | 0 | 0 | 0.244058 | 0 | 11 | 501.2107 | 0.793851 |
| 503 | −0.3635 | 0 | 0.280897 | 0.247949 | 0 | 0 | 11 | 507.1562 | 0.787972 |
| 504 | −0.3509 | −0.33426 | 0 | 0 | 0 | 0.230316 | 11 | 499.778 | 0.797526 |
| 505 | −0.35435 | 0 | 0.326983 | 0 | 0.276248 | 0 | 11 | 506.1088 | 0.78985 |
| 506 | −0.35429 | 0 | 0.335115 | 0 | 0 | 0.278056 | 11 | 504.427 | 0.792259 |
| 507 | −0.35918 | 0 | 0 | 0.257296 | 0.231108 | 0 | 11 | 506.4086 | 0.788237 |
| 508 | −0.36046 | 0 | 0 | 0.27483 | 0 | 0.241127 | 11 | 504.5215 | 0.791605 |
| 509 | −0.37665 | −0.49016 | 0.415897 | 0.213769 | 0 | 0 | 11 | 500.0235 | 0.79628 |
| 510 | −0.3692 | −0.49878 | 0.458935 | 0 | 0.257995 | 0 | 11 | 499.1148 | 0.796097 |
| 511 | −0.36789 | −0.4958 | 0.464972 | 0 | 0 | 0.255069 | 11 | 498.6496 | 0.797669 |
| 512 | −0.36683 | −0.32775 | 0 | 0.247282 | 0.215748 | 0 | 11 | 504.9741 | 0.788645 |
| 513 | −0.36769 | −0.32552 | 0 | 0.266766 | 0 | 0.223296 | 11 | 504.2837 | 0.79034 |
| 514 | −0.35346 | −0.32601 | 0 | 0 | 0.190682 | 0.171912 | 11 | 505.8092 | 0.788707 |
| 515 | −0.36573 | 0 | 0.297911 | 0.217151 | 0.249518 | 0 | 11 | 508.7905 | 0.785175 |
| 516 | −0.36751 | 0 | 0.304766 | 0.234822 | 0 | 0.26618 | 11 | 507.8045 | 0.7864 |
| 517 | −0.35702 | 0 | 0.341819 | 0 | 0.21209 | 0.213821 | 11 | 508.453 | 0.785869 |
| 518 | −0.36116 | 0 | 0 | 0.256551 | 0.172681 | 0.188346 | 11 | 509.8081 | 0.78299 |
| 519 | −0.37665 | −0.49016 | 0.415897 | 0.213769 | 0 | 0 | 11 | 495.2677 | 0.802772 |
| 520 | −0.3692 | −0.49878 | 0.458935 | 0 | 0.257995 | 0 | 11 | 493.8862 | 0.80412 |
| 521 | −0.36789 | −0.4958 | 0.464972 | 0 | 0 | 0.255069 | 11 | 492.2327 | 0.805753 |
| 522 | −0.36683 | −0.32775 | 0 | 0.247282 | 0.215748 | 0 | 11 | 499.1114 | 0.796035 |
| 523 | −0.36769 | −0.32552 | 0 | 0.266766 | 0 | 0.223296 | 11 | 497.185 | 0.79922 |
| 524 | −0.35346 | −0.32601 | 0 | 0 | 0.190682 | 0.171912 | 11 | 498.9488 | 0.797117 |
| 525 | −0.36573 | 0 | 0.297911 | 0.217151 | 0.249518 | 0 | 11 | 504.6417 | 0.791197 |
| 526 | −0.36751 | 0 | 0.304766 | 0.234822 | 0 | 0.26618 | 11 | 502.5838 | 0.793545 |
| 527 | −0.35702 | 0 | 0.341819 | 0 | 0.21209 | 0.213821 | 11 | 503.3284 | 0.792279 |
| 528 | −0.36116 | 0 | 0 | 0.256551 | 0.172681 | 0.188346 | 11 | 503.9664 | 0.791401 |
| 529 | −0.3784 | −0.48038 | 0.429094 | 0.184036 | 0.235336 | 0 | 11 | 498.2187 | 0.797607 |
| 530 | −0.37944 | −0.47686 | 0.432955 | 0.204998 | 0 | 0.247828 | 11 | 497.4709 | 0.799649 |
| 531 | −0.37057 | −0.48826 | 0.469023 | 0 | 0.198485 | 0.194003 | 11 | 497.7667 | 0.798016 |
| 532 | −0.36821 | −0.31823 | 0 | 0.248938 | 0.16128 | 0.17389 | 11 | 503.9818 | 0.790483 |
| 533 | −0.36844 | 0 | 0.31303 | 0.214129 | 0.186465 | 0.210249 | 11 | 507.1958 | 0.786685 |
| 534 | −0.34604 | 0 | 0 | 0 | 0.20186 | 0.189915 | 11 | 505.2779 | 0.791626 |
| 535 | −0.36484 | −0.51223 | 0.448932 | 0 | 0 | 0 | 11 | 496.3376 | 0.802936 |
| 536 | −0.36535 | −0.34122 | 0 | 0.272545 | 0 | 0 | 11 | 500.7116 | 0.795198 |
| 537 | −0.35247 | −0.33749 | 0 | 0 | 0.244058 | 0 | 11 | 500.4013 | 0.795688 |
| 538 | −0.3635 | 0 | 0.280897 | 0.247949 | 0 | 0 | 11 | 506.4927 | 0.789258 |
| 539 | −0.3509 | −0.33426 | 0 | 0 | 0 | 0.230316 | 11 | 499.3652 | 0.797485 |
| 540 | −0.35435 | 0 | 0.326983 | 0 | 0.276248 | 0 | 11 | 505.0014 | 0.792442 |
| 541 | −0.35429 | 0 | 0.335115 | 0 | 0 | 0.278056 | 11 | 503.7751 | 0.792687 |
| 542 | −0.35918 | 0 | 0 | 0.257296 | 0.231108 | 0 | 11 | 505.4355 | 0.789809 |
| 543 | −0.36046 | 0 | 0 | 0.27483 | 0 | 0.241127 | 11 | 503.9036 | 0.791952 |
| 544 | −0.37665 | −0.49016 | 0.415897 | 0.213769 | 0 | 0 | 11 | 498.5116 | 0.796648 |
| 545 | −0.3692 | −0.49878 | 0.458935 | 0 | 0.257995 | 0 | 11 | 496.8283 | 0.797995 |
| 546 | −0.36789 | −0.4958 | 0.464972 | 0 | 0 | 0.255069 | 11 | 497.0649 | 0.798812 |
| 547 | −0.36683 | −0.32775 | 0 | 0.247282 | 0.215748 | 0 | 11 | 503.0297 | 0.790748 |
| 548 | −0.36769 | −0.32552 | 0 | 0.266766 | 0 | 0.223296 | 11 | 502.8473 | 0.791463 |
| 549 | −0.35346 | −0.32601 | 0 | 0 | 0.190682 | 0.171912 | 11 | 503.9677 | 0.790197 |
| 550 | −0.36573 | 0 | 0.297911 | 0.217151 | 0.249518 | 0 | 11 | 506.6694 | 0.787073 |
| 551 | −0.36751 | 0 | 0.304766 | 0.234822 | 0 | 0.26618 | 11 | 506.2633 | 0.789013 |
| 552 | −0.35702 | 0 | 0.341819 | 0 | 0.21209 | 0.213821 | 11 | 506.3964 | 0.789421 |
| 553 | −0.36116 | 0 | 0 | 0.256551 | 0.172681 | 0.188346 | 11 | 507.962 | 0.786073 |
| 554 | −0.37665 | −0.49016 | 0.415897 | 0.213769 | 0 | 0 | 11 | 494.5157 | 0.803691 |
| 555 | −0.3692 | −0.49878 | 0.458935 | 0 | 0.257995 | 0 | 11 | 492.6139 | 0.80561 |
| 556 | −0.36789 | −0.4958 | 0.464972 | 0 | 0 | 0.255069 | 11 | 491.499 | 0.806345 |
| 557 | −0.36683 | −0.32775 | 0 | 0.247282 | 0.215748 | 0 | 11 | 498.0842 | 0.798118 |
| 558 | −0.36769 | −0.32552 | 0 | 0.266766 | 0 | 0.223296 | 11 | 496.541 | 0.800302 |
| 559 | −0.35346 | −0.32601 | 0 | 0 | 0.190682 | 0.171912 | 11 | 498.0546 | 0.798526 |
| 560 | −0.36573 | 0 | 0.297911 | 0.217151 | 0.249518 | 0 | 11 | 503.3632 | 0.793157 |
| 561 | −0.36751 | 0 | 0.304766 | 0.234822 | 0 | 0.26618 | 11 | 501.7687 | 0.794055 |
| 562 | −0.35702 | 0 | 0.341819 | 0 | 0.21209 | 0.213821 | 11 | 502.1472 | 0.79379 |
| 563 | −0.36116 | 0 | 0 | 0.256551 | 0.172681 | 0.188346 | 11 | 502.9475 | 0.792953 |
| 564 | −0.3784 | −0.48038 | 0.429094 | 0.184036 | 0.235336 | 0 | 11 | 495.7635 | 0.799955 |
| 565 | −0.37944 | −0.47686 | 0.432955 | 0.204998 | 0 | 0.247828 | 11 | 495.6885 | 0.800241 |
| 566 | −0.37057 | −0.48826 | 0.469023 | 0 | 0.198485 | 0.194003 | 11 | 495.4096 | 0.801078 |
| 567 | −0.36821 | −0.31823 | 0 | 0.248938 | 0.16128 | 0.17389 | 11 | 501.9978 | 0.792483 |
| 568 | −0.36844 | 0 | 0.31303 | 0.214129 | 0.186465 | 0.210249 | 11 | 505.0519 | 0.789748 |
| 569 | −0.37665 | −0.49016 | 0.415897 | 0.213769 | 0 | 0 | 11 | 494.8768 | 0.802323 |
| 570 | −0.3692 | −0.49878 | 0.458935 | 0 | 0.257995 | 0 | 11 | 493.5081 | 0.804855 |
| 571 | −0.36789 | −0.4958 | 0.464972 | 0 | 0 | 0.255069 | 11 | 491.7764 | 0.806712 |
| 572 | −0.36683 | −0.32775 | 0 | 0.247282 | 0.215748 | 0 | 11 | 498.5842 | 0.796709 |
| 573 | −0.36769 | −0.32552 | 0 | 0.266766 | 0 | 0.223296 | 11 | 496.5927 | 0.800588 |
| 574 | −0.35346 | −0.32601 | 0 | 0 | 0.190682 | 0.171912 | 11 | 498.3661 | 0.797464 |
| 575 | −0.36573 | 0 | 0.297911 | 0.217151 | 0.249518 | 0 | 11 | 504.018 | 0.791279 |
| 576 | −0.36751 | 0 | 0.304766 | 0.234822 | 0 | 0.26618 | 11 | 501.9141 | 0.794116 |
| 577 | −0.35702 | 0 | 0.341819 | 0 | 0.21209 | 0.213821 | 11 | 502.6419 | 0.793382 |
| 578 | −0.36116 | 0 | 0 | 0.256551 | 0.172681 | 0.188346 | 11 | 503.2443 | 0.793055 |
| 579 | −0.3784 | −0.48038 | 0.429094 | 0.184036 | 0.235336 | 0 | 11 | 497.7994 | 0.796954 |
| 580 | −0.37944 | −0.47686 | 0.432955 | 0.204998 | 0 | 0.247828 | 11 | 497.0512 | 0.799731 |
| 581 | −0.37057 | −0.48826 | 0.469023 | 0 | 0.198485 | 0.194003 | 11 | 497.3348 | 0.798281 |
| 582 | −0.36821 | −0.31823 | 0 | 0.248938 | 0.16128 | 0.17389 | 11 | 503.4477 | 0.790891 |
| 583 | −0.36844 | 0 | 0.31303 | 0.214129 | 0.186465 | 0.210249 | 11 | 506.7039 | 0.787604 |
| 584 | −0.3784 | −0.48038 | 0.429094 | 0.184036 | 0.235336 | 0 | 11 | 492.3445 | 0.80514 |
| 585 | −0.37944 | −0.47686 | 0.432955 | 0.204998 | 0 | 0.247828 | 11 | 490.2715 | 0.808203 |
| 586 | −0.37057 | −0.48826 | 0.469023 | 0 | 0.198485 | 0.194003 | 11 | 490.6716 | 0.807427 |
| 587 | −0.36821 | −0.31823 | 0 | 0.248938 | 0.16128 | 0.17389 | 11 | 496.0757 | 0.800098 |
| 588 | −0.36844 | 0 | 0.31303 | 0.214129 | 0.186465 | 0.210249 | 11 | 501.1556 | 0.795117 |
| 589 | −0.38017 | −0.47149 | 0.43946 | 0.184707 | 0.175726 | 0.194494 | 11 | 496.5413 | 0.799016 |
| 590 | −0.34604 | 0 | 0 | 0 | 0.20186 | 0.189915 | 11 | 508.9393 | 0.787318 |
| 591 | −0.36484 | −0.51223 | 0.448932 | 0 | 0 | 0 | 11 | 500.6943 | 0.797832 |
| 592 | −0.36535 | −0.34122 | 0 | 0.272545 | 0 | 0 | 11 | 504.2042 | 0.789993 |
| 593 | −0.35247 | −0.33749 | 0 | 0 | 0.244058 | 0 | 11 | 504.2327 | 0.790033 |
| 594 | −0.3635 | 0 | 0.280897 | 0.247949 | 0 | 0 | 11 | 509.9967 | 0.785461 |
| 595 | −0.3509 | −0.33426 | 0 | 0 | 0 | 0.230316 | 11 | 503.2004 | 0.792606 |
| 596 | −0.35435 | 0 | 0.326983 | 0 | 0.276248 | 0 | 11 | 508.998 | 0.787257 |
| 597 | −0.35429 | 0 | 0.335115 | 0 | 0 | 0.278056 | 11 | 507.7385 | 0.787237 |
| 598 | −0.35918 | 0 | 0 | 0.257296 | 0.231108 | 0 | 11 | 508.8575 | 0.7864 |
| 599 | −0.36046 | 0 | 0 | 0.27483 | 0 | 0.241127 | 11 | 507.3052 | 0.787339 |
| 600 | −0.37665 | −0.49016 | 0.415897 | 0.213769 | 0 | 0 | 11 | 502.2906 | 0.79283 |
| 601 | −0.3692 | −0.49878 | 0.458935 | 0 | 0.257995 | 0 | 11 | 501.0941 | 0.794014 |
| 602 | −0.36789 | −0.4958 | 0.464972 | 0 | 0 | 0.255069 | 11 | 501.0714 | 0.794423 |
| 603 | −0.36683 | −0.32775 | 0 | 0.247282 | 0.215748 | 0 | 11 | 506.2266 | 0.786461 |
| 604 | −0.36769 | −0.32552 | 0 | 0.266766 | 0 | 0.223296 | 11 | 505.8436 | 0.786604 |
| 605 | −0.35346 | −0.32601 | 0 | 0 | 0.190682 | 0.171912 | 11 | 507.2707 | 0.786951 |
| 606 | −0.36573 | 0 | 0.297911 | 0.217151 | 0.249518 | 0 | 11 | 510.1197 | 0.784583 |
| 607 | −0.36751 | 0 | 0.304766 | 0.234822 | 0 | 0.26618 | 11 | 509.4791 | 0.783419 |
| 608 | −0.35702 | 0 | 0.341819 | 0 | 0.21209 | 0.213821 | 11 | 510.0216 | 0.784338 |
| 609 | −0.36116 | 0 | 0 | 0.256551 | 0.172681 | 0.188346 | 11 | 510.878 | 0.782786 |
| 610 | −0.37665 | −0.49016 | 0.415897 | 0.213769 | 0 | 0 | 11 | 498.6216 | 0.798506 |
| 611 | −0.3692 | −0.49878 | 0.458935 | 0 | 0.257995 | 0 | 11 | 497.2752 | 0.799486 |
| 612 | −0.36789 | −0.4958 | 0.464972 | 0 | 0 | 0.255069 | 11 | 496.0985 | 0.801262 |
| 613 | −0.36683 | −0.32775 | 0 | 0.247282 | 0.215748 | 0 | 11 | 501.6572 | 0.793382 |
| 614 | −0.36769 | −0.32552 | 0 | 0.266766 | 0 | 0.223296 | 11 | 500.0867 | 0.794423 |
| 615 | −0.35346 | −0.32601 | 0 | 0 | 0.190682 | 0.171912 | 11 | 501.8737 | 0.793484 |
| 616 | −0.36573 | 0 | 0.297911 | 0.217151 | 0.249518 | 0 | 11 | 507.0168 | 0.788441 |
| 617 | −0.36751 | 0 | 0.304766 | 0.234822 | 0 | 0.26618 | 11 | 505.3519 | 0.789931 |
| 618 | −0.35702 | 0 | 0.341819 | 0 | 0.21209 | 0.213821 | 11 | 506.1382 | 0.789523 |
| 619 | −0.36116 | 0 | 0 | 0.256551 | 0.172681 | 0.188346 | 11 | 506.2582 | 0.788033 |
| 620 | −0.3784 | −0.48038 | 0.429094 | 0.184036 | 0.235336 | 0 | 11 | 499.6526 | 0.796097 |
| 621 | −0.37944 | −0.47686 | 0.432955 | 0.204998 | 0 | 0.247828 | 11 | 499.2905 | 0.795566 |
| 622 | −0.37057 | −0.48826 | 0.469023 | 0 | 0.198485 | 0.194003 | 11 | 499.4473 | 0.796219 |
| 623 | −0.36821 | −0.31823 | 0 | 0.248938 | 0.16128 | 0.17389 | 11 | 504.8599 | 0.789217 |
| 624 | −0.36844 | 0 | 0.31303 | 0.214129 | 0.186465 | 0.210249 | 11 | 508.1863 | 0.786685 |
| 625 | −0.37665 | −0.49016 | 0.415897 | 0.213769 | 0 | 0 | 11 | 499.4718 | 0.797526 |
| 626 | −0.3692 | −0.49878 | 0.458935 | 0 | 0.257995 | 0 | 11 | 498.7432 | 0.797791 |
| 627 | −0.36789 | −0.4958 | 0.464972 | 0 | 0 | 0.255069 | 11 | 496.8855 | 0.799935 |
| 628 | −0.36683 | −0.32775 | 0 | 0.247282 | 0.215748 | 0 | 11 | 502.7074 | 0.791952 |
| 629 | −0.36769 | −0.32552 | 0 | 0.266766 | 0 | 0.223296 | 11 | 500.6326 | 0.794076 |
| 630 | −0.35346 | −0.32601 | 0 | 0 | 0.190682 | 0.171912 | 11 | 502.745 | 0.792626 |
| 631 | −0.36573 | 0 | 0.297911 | 0.217151 | 0.249518 | 0 | 11 | 508.2555 | 0.786522 |
| 632 | −0.36751 | 0 | 0.304766 | 0.234822 | 0 | 0.26618 | 11 | 506.0367 | 0.788155 |
| 633 | −0.35702 | 0 | 0.341819 | 0 | 0.21209 | 0.213821 | 11 | 507.2322 | 0.786563 |
| 634 | −0.36116 | 0 | 0 | 0.256551 | 0.172681 | 0.188346 | 11 | 507.1251 | 0.786563 |
| 635 | −0.3784 | −0.48038 | 0.429094 | 0.184036 | 0.235336 | 0 | 11 | 502.1243 | 0.793259 |
| 636 | −0.37944 | −0.47686 | 0.432955 | 0.204998 | 0 | 0.247828 | 11 | 501.0657 | 0.794259 |
| 637 | −0.37057 | −0.48826 | 0.469023 | 0 | 0.198485 | 0.194003 | 11 | 501.8041 | 0.793239 |
| 638 | −0.36821 | −0.31823 | 0 | 0.248938 | 0.16128 | 0.17389 | 11 | 506.729 | 0.786155 |
| 639 | −0.36844 | 0 | 0.31303 | 0.214129 | 0.186465 | 0.210249 | 11 | 510.2573 | 0.782072 |
| 640 | −0.3784 | −0.48038 | 0.429094 | 0.184036 | 0.235336 | 0 | 11 | 497.1956 | 0.799771 |
| 641 | −0.37944 | −0.47686 | 0.432955 | 0.204998 | 0 | 0.247828 | 11 | 494.9828 | 0.801629 |
| 642 | −0.37057 | −0.48826 | 0.469023 | 0 | 0.198485 | 0.194003 | 11 | 495.8833 | 0.800486 |
| 643 | −0.36821 | −0.31823 | 0 | 0.248938 | 0.16128 | 0.17389 | 11 | 500.063 | 0.795362 |
| 644 | −0.36844 | 0 | 0.31303 | 0.214129 | 0.186465 | 0.210249 | 11 | 505.2378 | 0.788972 |
| 645 | −0.38017 | −0.47149 | 0.43946 | 0.184707 | 0.175726 | 0.194494 | 11 | 500.4245 | 0.795137 |
| 646 | −0.37665 | −0.49016 | 0.415897 | 0.213769 | 0 | 0 | 11 | 498.7063 | 0.798179 |
| 647 | −0.3692 | −0.49878 | 0.458935 | 0 | 0.257995 | 0 | 11 | 497.4429 | 0.799281 |
| 648 | −0.36789 | −0.4958 | 0.464972 | 0 | 0 | 0.255069 | 11 | 496.1133 | 0.801241 |
| 649 | −0.36683 | −0.32775 | 0 | 0.247282 | 0.215748 | 0 | 11 | 501.6418 | 0.792585 |
| 650 | −0.36769 | −0.32552 | 0 | 0.266766 | 0 | 0.223296 | 11 | 499.9366 | 0.794831 |
| 651 | −0.35346 | −0.32601 | 0 | 0 | 0.190682 | 0.171912 | 11 | 501.818 | 0.793667 |
| 652 | −0.36573 | 0 | 0.297911 | 0.217151 | 0.249518 | 0 | 11 | 506.8934 | 0.788237 |
| 653 | −0.36751 | 0 | 0.304766 | 0.234822 | 0 | 0.26618 | 11 | 505.1102 | 0.789319 |
| 654 | −0.35702 | 0 | 0.341819 | 0 | 0.21209 | 0.213821 | 11 | 505.9582 | 0.789564 |
| 655 | −0.36116 | 0 | 0 | 0.256551 | 0.172681 | 0.188346 | 11 | 505.9973 | 0.787931 |
| 656 | −0.3784 | −0.48038 | 0.429094 | 0.184036 | 0.235336 | 0 | 11 | 499.5164 | 0.795872 |
| 657 | −0.37944 | −0.47686 | 0.432955 | 0.204998 | 0 | 0.247828 | 11 | 499.067 | 0.795341 |
| 658 | −0.37057 | −0.48826 | 0.469023 | 0 | 0.198485 | 0.194003 | 11 | 499.2472 | 0.796485 |
| 659 | −0.36821 | −0.31823 | 0 | 0.248938 | 0.16128 | 0.17389 | 11 | 504.5083 | 0.789299 |
| 660 | −0.36844 | 0 | 0.31303 | 0.214129 | 0.186465 | 0.210249 | 11 | 507.8517 | 0.785787 |
| 661 | −0.3784 | −0.48038 | 0.429094 | 0.184036 | 0.235336 | 0 | 11 | 495.7244 | 0.800527 |
| 662 | −0.37944 | −0.47686 | 0.432955 | 0.204998 | 0 | 0.247828 | 11 | 493.9928 | 0.802017 |
| 663 | −0.37057 | −0.48826 | 0.469023 | 0 | 0.198485 | 0.194003 | 11 | 494.521 | 0.80167 |
| 664 | −0.36821 | −0.31823 | 0 | 0.248938 | 0.16128 | 0.17389 | 11 | 498.9342 | 0.796219 |
| 665 | −0.36844 | 0 | 0.31303 | 0.214129 | 0.186465 | 0.210249 | 11 | 503.8276 | 0.791707 |
| 666 | −0.38017 | −0.47149 | 0.43946 | 0.184707 | 0.175726 | 0.194494 | 11 | 497.7469 | 0.797383 |
| 667 | −0.3784 | −0.48038 | 0.429094 | 0.184036 | 0.235336 | 0 | 11 | 497.1212 | 0.799424 |
| 668 | −0.37944 | −0.47686 | 0.432955 | 0.204998 | 0 | 0.247828 | 11 | 494.8288 | 0.800976 |
| 669 | −0.37057 | −0.48826 | 0.469023 | 0 | 0.198485 | 0.194003 | 11 | 495.7635 | 0.800159 |
| 670 | −0.36821 | −0.31823 | 0 | 0.248938 | 0.16128 | 0.17389 | 11 | 499.8299 | 0.795137 |
| 671 | −0.36844 | 0 | 0.31303 | 0.214129 | 0.186465 | 0.210249 | 11 | 504.9327 | 0.788768 |
| 672 | −0.38017 | −0.47149 | 0.43946 | 0.184707 | 0.175726 | 0.194494 | 11 | 500.2386 | 0.794953 |
| 673 | −0.38017 | −0.47149 | 0.43946 | 0.184707 | 0.175726 | 0.194494 | 11 | 494.0586 | 0.802221 |
| 674 | −0.34604 | 0 | 0 | 0 | 0.20186 | 0.189915 | 11 | 552.2119 | 0.730973 |
| 675 | −0.36484 | −0.51223 | 0.448932 | 0 | 0 | 0 | 11 | 540.9972 | 0.743896 |
| 676 | −0.36535 | −0.34122 | 0 | 0.272545 | 0 | 0 | 11 | 552.7958 | 0.727054 |
| 677 | −0.35247 | −0.33749 | 0 | 0 | 0.244058 | 0 | 11 | 552.816 | 0.728687 |
| 678 | −0.3509 | −0.33426 | 0 | 0 | 0 | 0.230316 | 11 | 552.0145 | 0.729463 |
| 679 | −0.3635 | 0 | 0.280897 | 0.247949 | 0 | 0 | 11 | 546.705 | 0.737731 |
| 680 | −0.35435 | 0 | 0.326983 | 0 | 0.276248 | 0 | 11 | 544.2878 | 0.740283 |
| 681 | −0.35429 | 0 | 0.335115 | 0 | 0 | 0.278056 | 11 | 542.6122 | 0.744794 |
| 682 | −0.35918 | 0 | 0 | 0.257296 | 0.231108 | 0 | 11 | 551.6188 | 0.730851 |
| 683 | −0.36046 | 0 | 0 | 0.27483 | 0 | 0.241127 | 11 | 549.752 | 0.734403 |
| 684 | −0.37665 | −0.49016 | 0.415897 | 0.213769 | 0 | 0 | 11 | 540.9835 | 0.742283 |
| 685 | −0.3692 | −0.49878 | 0.458935 | 0 | 0.257995 | 0 | 11 | 538.2109 | 0.747264 |
| 686 | −0.36789 | −0.4958 | 0.464972 | 0 | 0 | 0.255069 | 11 | 538.3875 | 0.746734 |
| 687 | −0.36683 | −0.32775 | 0 | 0.247282 | 0.215748 | 0 | 11 | 552.4466 | 0.727891 |
| 688 | −0.36769 | −0.32552 | 0 | 0.266766 | 0 | 0.223296 | 11 | 552.0423 | 0.726503 |
| 689 | −0.35346 | −0.32601 | 0 | 0 | 0.190682 | 0.171912 | 11 | 554.0397 | 0.726972 |
| 690 | −0.36573 | 0 | 0.297911 | 0.217151 | 0.249518 | 0 | 11 | 544.2245 | 0.740752 |
| 691 | −0.36751 | 0 | 0.304766 | 0.234822 | 0 | 0.26618 | 11 | 543.3433 | 0.742487 |
| 692 | −0.35702 | 0 | 0.341819 | 0 | 0.21209 | 0.213821 | 11 | 543.3585 | 0.741773 |
| 693 | −0.36116 | 0 | 0 | 0.256551 | 0.172681 | 0.188346 | 11 | 552.1675 | 0.727585 |
| 694 | −0.37665 | −0.49016 | 0.415897 | 0.213769 | 0 | 0 | 11 | 538.7503 | 0.74704 |
| 695 | −0.3692 | −0.49878 | 0.458935 | 0 | 0.257995 | 0 | 11 | 535.666 | 0.751082 |
| 696 | −0.36789 | −0.4958 | 0.464972 | 0 | 0 | 0.255069 | 11 | 534.5782 | 0.752919 |
| 697 | −0.36683 | −0.32775 | 0 | 0.247282 | 0.215748 | 0 | 11 | 549.4505 | 0.732198 |
| 698 | −0.36769 | −0.32552 | 0 | 0.266766 | 0 | 0.223296 | 11 | 547.8992 | 0.733872 |
| 699 | −0.35346 | −0.32601 | 0 | 0 | 0.190682 | 0.171912 | 11 | 550.1627 | 0.730055 |
| 700 | −0.36573 | 0 | 0.297911 | 0.217151 | 0.249518 | 0 | 11 | 542.1404 | 0.743773 |
| 701 | −0.36751 | 0 | 0.304766 | 0.234822 | 0 | 0.26618 | 11 | 539.9638 | 0.748081 |
| 702 | −0.35702 | 0 | 0.341819 | 0 | 0.21209 | 0.213821 | 11 | 540.1423 | 0.746101 |
| 703 | −0.36116 | 0 | 0 | 0.256551 | 0.172681 | 0.188346 | 11 | 548.5453 | 0.734505 |
| 704 | −0.3784 | −0.48038 | 0.429094 | 0.184036 | 0.235336 | 0 | 11 | 536.81 | 0.749571 |
| 705 | −0.37944 | −0.47686 | 0.432955 | 0.204998 | 0 | 0.247828 | 11 | 536.4492 | 0.749551 |
| 706 | −0.37057 | −0.48826 | 0.469023 | 0 | 0.198485 | 0.194003 | 11 | 535.7898 | 0.749898 |
| 707 | −0.36821 | −0.31823 | 0 | 0.248938 | 0.16128 | 0.17389 | 11 | 550.8085 | 0.72883 |
| 708 | −0.36844 | 0 | 0.31303 | 0.214129 | 0.186465 | 0.210249 | 11 | 541.4103 | 0.743631 |
| 709 | −0.37665 | −0.49016 | 0.415897 | 0.213769 | 0 | 0 | 11 | 539.0297 | 0.74604 |
| 710 | −0.3692 | −0.49878 | 0.458935 | 0 | 0.257995 | 0 | 11 | 536.7134 | 0.748918 |
| 711 | −0.36789 | −0.4958 | 0.464972 | 0 | 0 | 0.255069 | 11 | 534.859 | 0.752041 |
| 712 | −0.36683 | −0.32775 | 0 | 0.247282 | 0.215748 | 0 | 11 | 549.7773 | 0.731443 |
| 713 | −0.36769 | −0.32552 | 0 | 0.266766 | 0 | 0.223296 | 11 | 547.7264 | 0.734873 |
| 714 | −0.35346 | −0.32601 | 0 | 0 | 0.190682 | 0.171912 | 11 | 550.3651 | 0.730198 |
| 715 | −0.36573 | 0 | 0.297911 | 0.217151 | 0.249518 | 0 | 11 | 542.9488 | 0.741017 |
| 716 | −0.36751 | 0 | 0.304766 | 0.234822 | 0 | 0.26618 | 11 | 540.0336 | 0.747183 |
| 717 | −0.35702 | 0 | 0.341819 | 0 | 0.21209 | 0.213821 | 11 | 540.8452 | 0.744733 |
| 718 | −0.36116 | 0 | 0 | 0.256551 | 0.172681 | 0.188346 | 11 | 548.7141 | 0.734709 |
| 719 | −0.3784 | −0.48038 | 0.429094 | 0.184036 | 0.235336 | 0 | 11 | 538.4787 | 0.746366 |
| 720 | −0.37944 | −0.47686 | 0.432955 | 0.204998 | 0 | 0.247828 | 11 | 537.3701 | 0.748183 |
| 721 | −0.37057 | −0.48826 | 0.469023 | 0 | 0.198485 | 0.194003 | 11 | 537.4181 | 0.747203 |
| 722 | −0.36821 | −0.31823 | 0 | 0.248938 | 0.16128 | 0.17389 | 11 | 551.6006 | 0.72689 |
| 723 | −0.36844 | 0 | 0.31303 | 0.214129 | 0.186465 | 0.210249 | 11 | 542.8106 | 0.741487 |
| 724 | −0.3784 | −0.48038 | 0.429094 | 0.184036 | 0.235336 | 0 | 11 | 535.2069 | 0.750102 |
| 725 | −0.37944 | −0.47686 | 0.432955 | 0.204998 | 0 | 0.247828 | 11 | 532.8432 | 0.754144 |
| 726 | −0.37057 | −0.48826 | 0.469023 | 0 | 0.198485 | 0.194003 | 11 | 532.9092 | 0.753491 |
| 727 | −0.36821 | −0.31823 | 0 | 0.248938 | 0.16128 | 0.17389 | 11 | 546.901 | 0.733974 |
| 728 | −0.36844 | 0 | 0.31303 | 0.214129 | 0.186465 | 0.210249 | 11 | 538.7124 | 0.746305 |
| 729 | −0.38017 | −0.47149 | 0.43946 | 0.184707 | 0.175726 | 0.194494 | 11 | 536.0474 | 0.748469 |
| 730 | −0.37665 | −0.49016 | 0.415897 | 0.213769 | 0 | 0 | 11 | 538.6384 | 0.747979 |
| 731 | −0.3692 | −0.49878 | 0.458935 | 0 | 0.257995 | 0 | 11 | 535.6705 | 0.752225 |
| 732 | −0.36789 | −0.4958 | 0.464972 | 0 | 0 | 0.255069 | 11 | 534.4434 | 0.753573 |
| 733 | −0.36683 | −0.32775 | 0 | 0.247282 | 0.215748 | 0 | 11 | 549.0351 | 0.734689 |
| 734 | −0.36769 | −0.32552 | 0 | 0.266766 | 0 | 0.223296 | 11 | 547.3436 | 0.736914 |
| 735 | −0.35346 | −0.32601 | 0 | 0 | 0.190682 | 0.171912 | 11 | 549.7401 | 0.733097 |
| 736 | −0.36573 | 0 | 0.297911 | 0.217151 | 0.249518 | 0 | 11 | 541.7843 | 0.743753 |
| 737 | −0.36751 | 0 | 0.304766 | 0.234822 | 0 | 0.26618 | 11 | 539.4424 | 0.748938 |
| 738 | −0.35702 | 0 | 0.341819 | 0 | 0.21209 | 0.213821 | 11 | 539.7616 | 0.747346 |
| 739 | −0.36116 | 0 | 0 | 0.256551 | 0.172681 | 0.188346 | 11 | 547.8742 | 0.736077 |
| 740 | −0.3784 | −0.48038 | 0.429094 | 0.184036 | 0.235336 | 0 | 11 | 536.5854 | 0.750388 |
| 741 | −0.37944 | −0.47686 | 0.432955 | 0.204998 | 0 | 0.247828 | 11 | 536.1378 | 0.750796 |
| 742 | −0.37057 | −0.48826 | 0.469023 | 0 | 0.198485 | 0.194003 | 11 | 535.5517 | 0.750082 |
| 743 | −0.36821 | −0.31823 | 0 | 0.248938 | 0.16128 | 0.17389 | 11 | 550.2169 | 0.729891 |
| 744 | −0.36844 | 0 | 0.31303 | 0.214129 | 0.186465 | 0.210249 | 11 | 540.9957 | 0.744406 |
| 745 | −0.3784 | −0.48038 | 0.429094 | 0.184036 | 0.235336 | 0 | 11 | 534.0192 | 0.753654 |
| 746 | −0.37944 | −0.47686 | 0.432955 | 0.204998 | 0 | 0.247828 | 11 | 532.2155 | 0.755941 |
| 747 | −0.37057 | −0.48826 | 0.469023 | 0 | 0.198485 | 0.194003 | 11 | 531.8301 | 0.755839 |
| 748 | −0.36821 | −0.31823 | 0 | 0.248938 | 0.16128 | 0.17389 | 11 | 546.0947 | 0.736383 |
| 749 | −0.36844 | 0 | 0.31303 | 0.214129 | 0.186465 | 0.210249 | 11 | 537.561 | 0.749755 |
| 750 | −0.38017 | −0.47149 | 0.43946 | 0.184707 | 0.175726 | 0.194494 | 11 | 534.0936 | 0.752572 |
| 751 | −0.3784 | −0.48038 | 0.429094 | 0.184036 | 0.235336 | 0 | 11 | 534.983 | 0.751572 |
| 752 | −0.37944 | −0.47686 | 0.432955 | 0.204998 | 0 | 0.247828 | 11 | 532.5383 | 0.756063 |
| 753 | −0.37057 | −0.48826 | 0.469023 | 0 | 0.198485 | 0.194003 | 11 | 532.6843 | 0.754818 |
| 754 | −0.36821 | −0.31823 | 0 | 0.248938 | 0.16128 | 0.17389 | 11 | 546.311 | 0.735934 |
| 755 | −0.36844 | 0 | 0.31303 | 0.214129 | 0.186465 | 0.210249 | 11 | 538.2355 | 0.749122 |
| 756 | −0.38017 | −0.47149 | 0.43946 | 0.184707 | 0.175726 | 0.194494 | 11 | 535.9046 | 0.750082 |
| 757 | −0.38017 | −0.47149 | 0.43946 | 0.184707 | 0.175726 | 0.194494 | 11 | 531.0544 | 0.755267 |
| 758 | −0.37665 | −0.49016 | 0.415897 | 0.213769 | 0 | 0 | 11 | 539.7448 | 0.745713 |
| 759 | −0.3692 | −0.49878 | 0.458935 | 0 | 0.257995 | 0 | 11 | 537.1631 | 0.747897 |
| 760 | −0.36789 | −0.4958 | 0.464972 | 0 | 0 | 0.255069 | 11 | 535.7709 | 0.751429 |
| 761 | −0.36683 | −0.32775 | 0 | 0.247282 | 0.215748 | 0 | 11 | 549.7379 | 0.732566 |
| 762 | −0.36769 | −0.32552 | 0 | 0.266766 | 0 | 0.223296 | 11 | 547.9761 | 0.735179 |
| 763 | −0.35346 | −0.32601 | 0 | 0 | 0.190682 | 0.171912 | 11 | 550.4735 | 0.731096 |
| 764 | −0.36573 | 0 | 0.297911 | 0.217151 | 0.249518 | 0 | 11 | 543.126 | 0.740793 |
| 765 | −0.36751 | 0 | 0.304766 | 0.234822 | 0 | 0.26618 | 11 | 540.642 | 0.745917 |
| 766 | −0.35702 | 0 | 0.341819 | 0 | 0.21209 | 0.213821 | 11 | 541.2683 | 0.744733 |
| 767 | −0.36116 | 0 | 0 | 0.256551 | 0.172681 | 0.188346 | 11 | 548.7179 | 0.734709 |
| 768 | −0.3784 | −0.48038 | 0.429094 | 0.184036 | 0.235336 | 0 | 11 | 537.905 | 0.748693 |
| 769 | −0.37944 | −0.47686 | 0.432955 | 0.204998 | 0 | 0.247828 | 11 | 537.1996 | 0.749285 |
| 770 | −0.37057 | −0.48826 | 0.469023 | 0 | 0.198485 | 0.194003 | 11 | 536.924 | 0.748387 |
| 771 | −0.36821 | −0.31823 | 0 | 0.248938 | 0.16128 | 0.17389 | 11 | 550.7848 | 0.72981 |
| 772 | −0.36844 | 0 | 0.31303 | 0.214129 | 0.186465 | 0.210249 | 11 | 542.1041 | 0.742181 |
| 773 | −0.3784 | −0.48038 | 0.429094 | 0.184036 | 0.235336 | 0 | 11 | 535.3539 | 0.753246 |
| 774 | −0.37944 | −0.47686 | 0.432955 | 0.204998 | 0 | 0.247828 | 11 | 533.3827 | 0.755573 |
| 775 | −0.37057 | −0.48826 | 0.469023 | 0 | 0.198485 | 0.194003 | 11 | 533.3156 | 0.753879 |
| 776 | −0.36821 | −0.31823 | 0 | 0.248938 | 0.16128 | 0.17389 | 11 | 546.7168 | 0.734648 |
| 777 | −0.36844 | 0 | 0.31303 | 0.214129 | 0.186465 | 0.210249 | 11 | 538.7981 | 0.747795 |
| 778 | −0.38017 | −0.47149 | 0.43946 | 0.184707 | 0.175726 | 0.194494 | 11 | 535.197 | 0.752144 |
| 779 | −0.3784 | −0.48038 | 0.429094 | 0.184036 | 0.235336 | 0 | 11 | 536.7245 | 0.749367 |
| 780 | −0.37944 | −0.47686 | 0.432955 | 0.204998 | 0 | 0.247828 | 11 | 534.0389 | 0.753307 |
| 781 | −0.37057 | −0.48826 | 0.469023 | 0 | 0.198485 | 0.194003 | 11 | 534.583 | 0.750061 |
| 782 | −0.36821 | −0.31823 | 0 | 0.248938 | 0.16128 | 0.17389 | 11 | 547.2607 | 0.734036 |
| 783 | −0.36844 | 0 | 0.31303 | 0.214129 | 0.186465 | 0.210249 | 11 | 539.8491 | 0.7451 |
| 784 | −0.38017 | −0.47149 | 0.43946 | 0.184707 | 0.175726 | 0.194494 | 11 | 537.204 | 0.747571 |
| 785 | −0.38017 | −0.47149 | 0.43946 | 0.184707 | 0.175726 | 0.194494 | 11 | 532.6783 | 0.754389 |
| 786 | −0.3784 | −0.48038 | 0.429094 | 0.184036 | 0.235336 | 0 | 11 | 535.4984 | 0.752491 |
| 787 | −0.37944 | −0.47686 | 0.432955 | 0.204998 | 0 | 0.247828 | 11 | 533.38 | 0.755777 |
| 788 | −0.37057 | −0.48826 | 0.469023 | 0 | 0.198485 | 0.194003 | 11 | 533.4508 | 0.754512 |
| 789 | −0.36821 | −0.31823 | 0 | 0.248938 | 0.16128 | 0.17389 | 11 | 546.4272 | 0.736812 |
| 790 | −0.36844 | 0 | 0.31303 | 0.214129 | 0.186465 | 0.210249 | 11 | 538.5818 | 0.748775 |
| 791 | −0.38017 | −0.47149 | 0.43946 | 0.184707 | 0.175726 | 0.194494 | 11 | 535.0617 | 0.752491 |
| 792 | −0.38017 | −0.47149 | 0.43946 | 0.184707 | 0.175726 | 0.194494 | 11 | 531.4087 | 0.757513 |
| 793 | −0.38017 | −0.47149 | 0.43946 | 0.184707 | 0.175726 | 0.194494 | 11 | 532.5849 | 0.754614 |
| 794 | −0.38017 | −0.47149 | 0.43946 | 0.184707 | 0.175726 | 0.194494 | 11 | 554.981 | 0.718745 |
| 795 | −0.34954 | 0 | 0.311734 | 0 | 0 | 0 | 10 | 549.7743 | 0.728891 |
| 796 | −0.34838 | −0.35299 | 0 | 0 | 0 | 0 | 10 | 536.8024 | 0.751327 |
| 797 | −0.35731 | 0 | 0 | 0.283735 | 0 | 0 | 10 | 546.5068 | 0.737465 |
| 798 | −0.34421 | 0 | 0 | 0 | 0.260301 | 0 | 10 | 544.2941 | 0.73771 |
| 799 | −0.34349 | 0 | 0 | 0 | 0 | 0.25144 | 10 | 544.2333 | 0.740058 |
| 800 | −0.34604 | 0 | 0 | 0 | 0.20186 | 0.189915 | 10 | 546.7917 | 0.734709 |
| 801 | −0.36484 | −0.51223 | 0.448932 | 0 | 0 | 0 | 10 | 534.0529 | 0.752286 |
| 802 | −0.36535 | −0.34122 | 0 | 0.272545 | 0 | 0 | 10 | 537.8115 | 0.751 |
| 803 | −0.35247 | −0.33749 | 0 | 0 | 0.244058 | 0 | 10 | 536.3938 | 0.750204 |
| 804 | −0.3635 | 0 | 0.280897 | 0.247949 | 0 | 0 | 10 | 549.6745 | 0.728952 |
| 805 | −0.3509 | −0.33426 | 0 | 0 | 0 | 0.230316 | 10 | 538.2761 | 0.748775 |
| 806 | −0.35435 | 0 | 0.326983 | 0 | 0.276248 | 0 | 10 | 546.3839 | 0.733893 |
| 807 | −0.35429 | 0 | 0.335115 | 0 | 0 | 0.278056 | 10 | 548.1574 | 0.733464 |
| 808 | −0.35918 | 0 | 0 | 0.257296 | 0.231108 | 0 | 10 | 545.4368 | 0.736608 |
| 809 | −0.36046 | 0 | 0 | 0.27483 | 0 | 0.241127 | 10 | 546.3167 | 0.738302 |
| 810 | −0.34604 | 0 | 0 | 0 | 0.20186 | 0.189915 | 10 | 541.4428 | 0.744059 |
| 811 | −0.36484 | −0.51223 | 0.448932 | 0 | 0 | 0 | 10 | 530.2903 | 0.75888 |
| 812 | −0.36535 | −0.34122 | 0 | 0.272545 | 0 | 0 | 10 | 533.1613 | 0.759105 |
| 813 | −0.35247 | −0.33749 | 0 | 0 | 0.244058 | 0 | 10 | 531.3881 | 0.758084 |
| 814 | −0.3635 | 0 | 0.280897 | 0.247949 | 0 | 0 | 10 | 546.8576 | 0.734321 |
| 815 | −0.3509 | −0.33426 | 0 | 0 | 0 | 0.230316 | 10 | 531.4575 | 0.759697 |
| 816 | −0.35435 | 0 | 0.326983 | 0 | 0.276248 | 0 | 10 | 543.3037 | 0.740038 |
| 817 | −0.35429 | 0 | 0.335115 | 0 | 0 | 0.278056 | 10 | 543.3851 | 0.739833 |
| 818 | −0.35918 | 0 | 0 | 0.257296 | 0.231108 | 0 | 10 | 541.5083 | 0.743957 |
| 819 | −0.36046 | 0 | 0 | 0.27483 | 0 | 0.241127 | 10 | 540.701 | 0.745529 |
| 820 | −0.37665 | −0.49016 | 0.415897 | 0.213769 | 0 | 0 | 10 | 531.9043 | 0.757431 |
| 821 | −0.3692 | −0.49878 | 0.458935 | 0 | 0.257995 | 0 | 10 | 528.2813 | 0.760595 |
| 822 | −0.36789 | −0.4958 | 0.464972 | 0 | 0 | 0.255069 | 10 | 530.4997 | 0.754001 |
| 823 | −0.36683 | −0.32775 | 0 | 0.247282 | 0.215748 | 0 | 10 | 533.8654 | 0.755532 |
| 824 | −0.36769 | −0.32552 | 0 | 0.266766 | 0 | 0.223296 | 10 | 535.0447 | 0.75494 |
| 825 | −0.35346 | −0.32601 | 0 | 0 | 0.190682 | 0.171912 | 10 | 535.6668 | 0.751368 |
| 826 | −0.36573 | 0 | 0.297911 | 0.217151 | 0.249518 | 0 | 10 | 544.486 | 0.738078 |
| 827 | −0.36751 | 0 | 0.304766 | 0.234822 | 0 | 0.26618 | 10 | 545.6213 | 0.737976 |
| 828 | −0.35702 | 0 | 0.341819 | 0 | 0.21209 | 0.213821 | 10 | 544.8009 | 0.738751 |
| 829 | −0.36116 | 0 | 0 | 0.256551 | 0.172681 | 0.188346 | 10 | 544.1684 | 0.740487 |
| 830 | −0.34604 | 0 | 0 | 0 | 0.20186 | 0.189915 | 10 | 540.8196 | 0.744794 |
| 831 | −0.36484 | −0.51223 | 0.448932 | 0 | 0 | 0 | 10 | 529.8305 | 0.759942 |
| 832 | −0.36535 | −0.34122 | 0 | 0.272545 | 0 | 0 | 10 | 532.5705 | 0.759554 |
| 833 | −0.35247 | −0.33749 | 0 | 0 | 0.244058 | 0 | 10 | 531.1813 | 0.757002 |
| 834 | −0.3635 | 0 | 0.280897 | 0.247949 | 0 | 0 | 10 | 546.1437 | 0.734668 |
| 835 | −0.3509 | −0.33426 | 0 | 0 | 0 | 0.230316 | 10 | 530.6522 | 0.759717 |
| 836 | −0.35435 | 0 | 0.326983 | 0 | 0.276248 | 0 | 10 | 543.016 | 0.738568 |
| 837 | −0.35429 | 0 | 0.335115 | 0 | 0 | 0.278056 | 10 | 542.5109 | 0.741242 |
| 838 | −0.35918 | 0 | 0 | 0.257296 | 0.231108 | 0 | 10 | 541.1803 | 0.743059 |
| 839 | −0.36046 | 0 | 0 | 0.27483 | 0 | 0.241127 | 10 | 539.8865 | 0.747754 |
| 840 | −0.37665 | −0.49016 | 0.415897 | 0.213769 | 0 | 0 | 10 | 532.0926 | 0.757411 |
| 841 | −0.3692 | −0.49878 | 0.458935 | 0 | 0.257995 | 0 | 10 | 529.3108 | 0.758288 |
| 842 | −0.36789 | −0.4958 | 0.464972 | 0 | 0 | 0.255069 | 10 | 530.6614 | 0.756716 |
| 843 | −0.36683 | −0.32775 | 0 | 0.247282 | 0.215748 | 0 | 10 | 534.4888 | 0.754226 |
| 844 | −0.36769 | −0.32552 | 0 | 0.266766 | 0 | 0.223296 | 10 | 535.0099 | 0.754634 |
| 845 | −0.35346 | −0.32601 | 0 | 0 | 0.190682 | 0.171912 | 10 | 536.0354 | 0.751143 |
| 846 | −0.36573 | 0 | 0.297911 | 0.217151 | 0.249518 | 0 | 10 | 545.0179 | 0.736261 |
| 847 | −0.36751 | 0 | 0.304766 | 0.234822 | 0 | 0.26618 | 10 | 545.4942 | 0.737792 |
| 848 | −0.35702 | 0 | 0.341819 | 0 | 0.21209 | 0.213821 | 10 | 545.1446 | 0.736016 |
| 849 | −0.36116 | 0 | 0 | 0.256551 | 0.172681 | 0.188346 | 10 | 544.4166 | 0.737792 |
| 850 | −0.37665 | −0.49016 | 0.415897 | 0.213769 | 0 | 0 | 10 | 527.6062 | 0.764005 |
| 851 | −0.3692 | −0.49878 | 0.458935 | 0 | 0.257995 | 0 | 10 | 524.2112 | 0.767148 |
| 852 | −0.36789 | −0.4958 | 0.464972 | 0 | 0 | 0.255069 | 10 | 523.7808 | 0.766128 |
| 853 | −0.36683 | −0.32775 | 0 | 0.247282 | 0.215748 | 0 | 10 | 528.518 | 0.763535 |
| 854 | −0.36769 | −0.32552 | 0 | 0.266766 | 0 | 0.223296 | 10 | 527.2077 | 0.76672 |
| 855 | −0.35346 | −0.32601 | 0 | 0 | 0.190682 | 0.171912 | 10 | 528.5131 | 0.761085 |
| 856 | −0.36573 | 0 | 0.297911 | 0.217151 | 0.249518 | 0 | 10 | 541.0999 | 0.743488 |
| 857 | −0.36751 | 0 | 0.304766 | 0.234822 | 0 | 0.26618 | 10 | 539.9217 | 0.745529 |
| 858 | −0.35702 | 0 | 0.341819 | 0 | 0.21209 | 0.213821 | 10 | 539.6644 | 0.743998 |
| 859 | −0.36116 | 0 | 0 | 0.256551 | 0.172681 | 0.188346 | 10 | 538.0612 | 0.748938 |
| 860 | −0.3784 | −0.48038 | 0.429094 | 0.184036 | 0.235336 | 0 | 10 | 528.0689 | 0.761371 |
| 861 | −0.37944 | −0.47686 | 0.432955 | 0.204998 | 0 | 0.247828 | 10 | 528.8631 | 0.760902 |
| 862 | −0.37057 | −0.48826 | 0.469023 | 0 | 0.198485 | 0.194003 | 10 | 528.0776 | 0.760167 |
| 863 | −0.36821 | −0.31823 | 0 | 0.248938 | 0.16128 | 0.17389 | 10 | 533.6229 | 0.755777 |
| 864 | −0.36844 | 0 | 0.31303 | 0.214129 | 0.186465 | 0.210249 | 10 | 543.4264 | 0.739098 |
| 865 | −0.34604 | 0 | 0 | 0 | 0.20186 | 0.189915 | 10 | 541.2348 | 0.743712 |
| 866 | −0.36484 | −0.51223 | 0.448932 | 0 | 0 | 0 | 10 | 530.8218 | 0.756941 |
| 867 | −0.36535 | −0.34122 | 0 | 0.272545 | 0 | 0 | 10 | 533.2139 | 0.758003 |
| 868 | −0.35247 | −0.33749 | 0 | 0 | 0.244058 | 0 | 10 | 531.666 | 0.757227 |
| 869 | −0.3635 | 0 | 0.280897 | 0.247949 | 0 | 0 | 10 | 546.7999 | 0.733934 |
| 870 | −0.3509 | −0.33426 | 0 | 0 | 0 | 0.230316 | 10 | 531.4209 | 0.759125 |
| 871 | −0.35435 | 0 | 0.326983 | 0 | 0.276248 | 0 | 10 | 543.4225 | 0.7382 |
| 872 | −0.35429 | 0 | 0.335115 | 0 | 0 | 0.278056 | 10 | 543.1938 | 0.739874 |
| 873 | −0.35918 | 0 | 0 | 0.257296 | 0.231108 | 0 | 10 | 541.3823 | 0.743467 |
| 874 | −0.36046 | 0 | 0 | 0.27483 | 0 | 0.241127 | 10 | 540.2808 | 0.746877 |
| 875 | −0.37665 | −0.49016 | 0.415897 | 0.213769 | 0 | 0 | 10 | 531.7844 | 0.758003 |
| 876 | −0.3692 | −0.49878 | 0.458935 | 0 | 0.257995 | 0 | 10 | 528.2822 | 0.75937 |
| 877 | −0.36789 | −0.4958 | 0.464972 | 0 | 0 | 0.255069 | 10 | 530.172 | 0.755777 |
| 878 | −0.36683 | −0.32775 | 0 | 0.247282 | 0.215748 | 0 | 10 | 533.41 | 0.755471 |
| 879 | −0.36769 | −0.32552 | 0 | 0.266766 | 0 | 0.223296 | 10 | 534.3124 | 0.755308 |
| 880 | −0.35346 | −0.32601 | 0 | 0 | 0.190682 | 0.171912 | 10 | 535.0445 | 0.751776 |
| 881 | −0.36573 | 0 | 0.297911 | 0.217151 | 0.249518 | 0 | 10 | 544.006 | 0.737506 |
| 882 | −0.36751 | 0 | 0.304766 | 0.234822 | 0 | 0.26618 | 10 | 544.8598 | 0.738466 |
| 883 | −0.35702 | 0 | 0.341819 | 0 | 0.21209 | 0.213821 | 10 | 544.1346 | 0.738506 |
| 884 | −0.36116 | 0 | 0 | 0.256551 | 0.172681 | 0.188346 | 10 | 543.3096 | 0.740119 |
| 885 | −0.37665 | −0.49016 | 0.415897 | 0.213769 | 0 | 0 | 10 | 528.0502 | 0.763433 |
| 886 | −0.3692 | −0.49878 | 0.458935 | 0 | 0.257995 | 0 | 10 | 524.3092 | 0.765781 |
| 887 | −0.36789 | −0.4958 | 0.464972 | 0 | 0 | 0.255069 | 10 | 524.2554 | 0.76525 |
| 888 | −0.36683 | −0.32775 | 0 | 0.247282 | 0.215748 | 0 | 10 | 528.4766 | 0.763596 |
| 889 | −0.36769 | −0.32552 | 0 | 0.266766 | 0 | 0.223296 | 10 | 527.4064 | 0.766679 |
| 890 | −0.35346 | −0.32601 | 0 | 0 | 0.190682 | 0.171912 | 10 | 528.6961 | 0.760799 |
| 891 | −0.36573 | 0 | 0.297911 | 0.217151 | 0.249518 | 0 | 10 | 541.0107 | 0.742998 |
| 892 | −0.36751 | 0 | 0.304766 | 0.234822 | 0 | 0.26618 | 10 | 540.0933 | 0.745529 |
| 893 | −0.35702 | 0 | 0.341819 | 0 | 0.21209 | 0.213821 | 10 | 539.7168 | 0.745325 |
| 894 | −0.36116 | 0 | 0 | 0.256551 | 0.172681 | 0.188346 | 10 | 537.9555 | 0.750327 |
| 895 | −0.3784 | −0.48038 | 0.429094 | 0.184036 | 0.235336 | 0 | 10 | 526.6517 | 0.76329 |
| 896 | −0.37944 | −0.47686 | 0.432955 | 0.204998 | 0 | 0.247828 | 10 | 527.9419 | 0.760677 |
| 897 | −0.37057 | −0.48826 | 0.469023 | 0 | 0.198485 | 0.194003 | 10 | 526.7011 | 0.76131 |
| 898 | −0.36821 | −0.31823 | 0 | 0.248938 | 0.16128 | 0.17389 | 10 | 532.2462 | 0.758207 |
| 899 | −0.36844 | 0 | 0.31303 | 0.214129 | 0.186465 | 0.210249 | 10 | 542.0716 | 0.742222 |
| 900 | −0.37665 | −0.49016 | 0.415897 | 0.213769 | 0 | 0 | 10 | 527.7627 | 0.762882 |
| 901 | −0.3692 | −0.49878 | 0.458935 | 0 | 0.257995 | 0 | 10 | 524.4309 | 0.765393 |
| 902 | −0.36789 | −0.4958 | 0.464972 | 0 | 0 | 0.255069 | 10 | 523.8483 | 0.766066 |
| 903 | −0.36683 | −0.32775 | 0 | 0.247282 | 0.215748 | 0 | 10 | 528.3683 | 0.762718 |
| 904 | −0.36769 | −0.32552 | 0 | 0.266766 | 0 | 0.223296 | 10 | 526.8661 | 0.767883 |
| 905 | −0.35346 | −0.32601 | 0 | 0 | 0.190682 | 0.171912 | 10 | 528.3283 | 0.761596 |
| 906 | −0.36573 | 0 | 0.297911 | 0.217151 | 0.249518 | 0 | 10 | 540.8159 | 0.741487 |
| 907 | −0.36751 | 0 | 0.304766 | 0.234822 | 0 | 0.26618 | 10 | 539.4625 | 0.745529 |
| 908 | −0.35702 | 0 | 0.341819 | 0 | 0.21209 | 0.213821 | 10 | 539.2938 | 0.744794 |
| 909 | −0.36116 | 0 | 0 | 0.256551 | 0.172681 | 0.188346 | 10 | 537.5117 | 0.749428 |
| 910 | −0.3784 | −0.48038 | 0.429094 | 0.184036 | 0.235336 | 0 | 10 | 527.6243 | 0.761187 |
| 911 | −0.37944 | −0.47686 | 0.432955 | 0.204998 | 0 | 0.247828 | 10 | 528.2506 | 0.761596 |
| 912 | −0.37057 | −0.48826 | 0.469023 | 0 | 0.198485 | 0.194003 | 10 | 527.5046 | 0.759983 |
| 913 | −0.36821 | −0.31823 | 0 | 0.248938 | 0.16128 | 0.17389 | 10 | 532.756 | 0.756614 |
| 914 | −0.36844 | 0 | 0.31303 | 0.214129 | 0.186465 | 0.210249 | 10 | 542.5066 | 0.740528 |
| 915 | −0.3784 | −0.48038 | 0.429094 | 0.184036 | 0.235336 | 0 | 10 | 522.6821 | 0.768639 |
| 916 | −0.37944 | −0.47686 | 0.432955 | 0.204998 | 0 | 0.247828 | 10 | 521.3933 | 0.77117 |
| 917 | −0.37057 | −0.48826 | 0.469023 | 0 | 0.198485 | 0.194003 | 10 | 520.8987 | 0.768496 |
| 918 | −0.36821 | −0.31823 | 0 | 0.248938 | 0.16128 | 0.17389 | 10 | 525.1208 | 0.768353 |
| 919 | −0.36844 | 0 | 0.31303 | 0.214129 | 0.186465 | 0.210249 | 10 | 537.0479 | 0.748408 |
| 920 | −0.38017 | −0.47149 | 0.43946 | 0.184707 | 0.175726 | 0.194494 | 10 | 526.1337 | 0.763555 |
| 921 | −0.34604 | 0 | 0 | 0 | 0.20186 | 0.189915 | 10 | 545.6832 | 0.734158 |
| 922 | −0.36484 | −0.51223 | 0.448932 | 0 | 0 | 0 | 10 | 535.8391 | 0.750041 |
| 923 | −0.36535 | −0.34122 | 0 | 0.272545 | 0 | 0 | 10 | 537.3221 | 0.752225 |
| 924 | −0.35247 | −0.33749 | 0 | 0 | 0.244058 | 0 | 10 | 536.2903 | 0.750122 |
| 925 | −0.3635 | 0 | 0.280897 | 0.247949 | 0 | 0 | 10 | 550.8899 | 0.725951 |
| 926 | −0.3509 | −0.33426 | 0 | 0 | 0 | 0.230316 | 10 | 535.987 | 0.75149 |
| 927 | −0.35435 | 0 | 0.326983 | 0 | 0.276248 | 0 | 10 | 548.2691 | 0.728993 |
| 928 | −0.35429 | 0 | 0.335115 | 0 | 0 | 0.278056 | 10 | 547.9095 | 0.729585 |
| 929 | −0.35918 | 0 | 0 | 0.257296 | 0.231108 | 0 | 10 | 545.4887 | 0.73622 |
| 930 | −0.36046 | 0 | 0 | 0.27483 | 0 | 0.241127 | 10 | 544.3345 | 0.738323 |
| 931 | −0.37665 | −0.49016 | 0.415897 | 0.213769 | 0 | 0 | 10 | 536.5023 | 0.750694 |
| 932 | −0.3692 | −0.49878 | 0.458935 | 0 | 0.257995 | 0 | 10 | 533.7965 | 0.750919 |
| 933 | −0.36789 | −0.4958 | 0.464972 | 0 | 0 | 0.255069 | 10 | 535.2841 | 0.749122 |
| 934 | −0.36683 | −0.32775 | 0 | 0.247282 | 0.215748 | 0 | 10 | 537.6609 | 0.749673 |
| 935 | −0.36769 | −0.32552 | 0 | 0.266766 | 0 | 0.223296 | 10 | 538.2887 | 0.749898 |
| 936 | −0.35346 | −0.32601 | 0 | 0 | 0.190682 | 0.171912 | 10 | 539.5011 | 0.746162 |
| 937 | −0.36573 | 0 | 0.297911 | 0.217151 | 0.249518 | 0 | 10 | 548.6779 | 0.729401 |
| 938 | −0.36751 | 0 | 0.304766 | 0.234822 | 0 | 0.26618 | 10 | 549.1997 | 0.729748 |
| 939 | −0.35702 | 0 | 0.341819 | 0 | 0.21209 | 0.213821 | 10 | 549.13 | 0.72687 |
| 940 | −0.36116 | 0 | 0 | 0.256551 | 0.172681 | 0.188346 | 10 | 547.3683 | 0.732709 |
| 941 | −0.37665 | −0.49016 | 0.415897 | 0.213769 | 0 | 0 | 10 | 532.7355 | 0.756716 |
| 942 | −0.3692 | −0.49878 | 0.458935 | 0 | 0.257995 | 0 | 10 | 529.7856 | 0.757635 |
| 943 | −0.36789 | −0.4958 | 0.464972 | 0 | 0 | 0.255069 | 10 | 529.5401 | 0.758043 |
| 944 | −0.36683 | −0.32775 | 0 | 0.247282 | 0.215748 | 0 | 10 | 532.7299 | 0.758023 |
| 945 | −0.36769 | −0.32552 | 0 | 0.266766 | 0 | 0.223296 | 10 | 531.5854 | 0.760657 |
| 946 | −0.35346 | −0.32601 | 0 | 0 | 0.190682 | 0.171912 | 10 | 533.2322 | 0.755287 |
| 947 | −0.36573 | 0 | 0.297911 | 0.217151 | 0.249518 | 0 | 10 | 545.3692 | 0.735608 |
| 948 | −0.36751 | 0 | 0.304766 | 0.234822 | 0 | 0.26618 | 10 | 544.3106 | 0.737976 |
| 949 | −0.35702 | 0 | 0.341819 | 0 | 0.21209 | 0.213821 | 10 | 544.4996 | 0.734995 |
| 950 | −0.36116 | 0 | 0 | 0.256551 | 0.172681 | 0.188346 | 10 | 541.9241 | 0.742283 |
| 951 | −0.3784 | −0.48038 | 0.429094 | 0.184036 | 0.235336 | 0 | 10 | 531.676 | 0.756349 |
| 952 | −0.37944 | −0.47686 | 0.432955 | 0.204998 | 0 | 0.247828 | 10 | 532.5416 | 0.754716 |
| 953 | −0.37057 | −0.48826 | 0.469023 | 0 | 0.198485 | 0.194003 | 10 | 531.9571 | 0.753573 |
| 954 | −0.36821 | −0.31823 | 0 | 0.248938 | 0.16128 | 0.17389 | 10 | 536.1203 | 0.752797 |
| 955 | −0.36844 | 0 | 0.31303 | 0.214129 | 0.186465 | 0.210249 | 10 | 546.3972 | 0.733199 |
| 956 | −0.37665 | −0.49016 | 0.415897 | 0.213769 | 0 | 0 | 10 | 533.0078 | 0.755798 |
| 957 | −0.3692 | −0.49878 | 0.458935 | 0 | 0.257995 | 0 | 10 | 530.6823 | 0.756124 |
| 958 | −0.36789 | −0.4958 | 0.464972 | 0 | 0 | 0.255069 | 10 | 529.7444 | 0.757533 |
| 959 | −0.36683 | −0.32775 | 0 | 0.247282 | 0.215748 | 0 | 10 | 533.3592 | 0.755512 |
| 960 | −0.36769 | −0.32552 | 0 | 0.266766 | 0 | 0.223296 | 10 | 531.654 | 0.760371 |
| 961 | −0.35346 | −0.32601 | 0 | 0 | 0.190682 | 0.171912 | 10 | 533.6485 | 0.753042 |
| 962 | −0.36573 | 0 | 0.297911 | 0.217151 | 0.249518 | 0 | 10 | 545.9464 | 0.73279 |
| 963 | −0.36751 | 0 | 0.304766 | 0.234822 | 0 | 0.26618 | 10 | 544.3361 | 0.736261 |
| 964 | −0.35702 | 0 | 0.341819 | 0 | 0.21209 | 0.213821 | 10 | 544.9027 | 0.732954 |
| 965 | −0.36116 | 0 | 0 | 0.256551 | 0.172681 | 0.188346 | 10 | 542.2502 | 0.739323 |
| 966 | −0.3784 | −0.48038 | 0.429094 | 0.184036 | 0.235336 | 0 | 10 | 533.4261 | 0.752144 |
| 967 | −0.37944 | −0.47686 | 0.432955 | 0.204998 | 0 | 0.247828 | 10 | 533.5617 | 0.75296 |
| 968 | −0.37057 | −0.48826 | 0.469023 | 0 | 0.198485 | 0.194003 | 10 | 533.5803 | 0.750939 |
| 969 | −0.36821 | −0.31823 | 0 | 0.248938 | 0.16128 | 0.17389 | 10 | 537.3972 | 0.749775 |
| 970 | −0.36844 | 0 | 0.31303 | 0.214129 | 0.186465 | 0.210249 | 10 | 547.6314 | 0.730626 |
| 971 | −0.3784 | −0.48038 | 0.429094 | 0.184036 | 0.235336 | 0 | 10 | 528.4713 | 0.760248 |
| 972 | −0.37944 | −0.47686 | 0.432955 | 0.204998 | 0 | 0.247828 | 10 | 526.8651 | 0.764699 |
| 973 | −0.37057 | −0.48826 | 0.469023 | 0 | 0.198485 | 0.194003 | 10 | 527.1038 | 0.760187 |
| 974 | −0.36821 | −0.31823 | 0 | 0.248938 | 0.16128 | 0.17389 | 10 | 529.9622 | 0.760248 |
| 975 | −0.36844 | 0 | 0.31303 | 0.214129 | 0.186465 | 0.210249 | 10 | 542.0258 | 0.738302 |
| 976 | −0.38017 | −0.47149 | 0.43946 | 0.184707 | 0.175726 | 0.194494 | 10 | 531.5181 | 0.755308 |
| 977 | −0.37665 | −0.49016 | 0.415897 | 0.213769 | 0 | 0 | 10 | 533.8646 | 0.754042 |
| 978 | −0.3692 | −0.49878 | 0.458935 | 0 | 0.257995 | 0 | 10 | 531.1458 | 0.755063 |
| 979 | −0.36789 | −0.4958 | 0.464972 | 0 | 0 | 0.255069 | 10 | 530.6135 | 0.756329 |
| 980 | −0.36683 | −0.32775 | 0 | 0.247282 | 0.215748 | 0 | 10 | 533.6497 | 0.756308 |
| 981 | −0.36769 | −0.32552 | 0 | 0.266766 | 0 | 0.223296 | 10 | 532.193 | 0.759758 |
| 982 | −0.35346 | −0.32601 | 0 | 0 | 0.190682 | 0.171912 | 10 | 534.1986 | 0.752981 |
| 983 | −0.36573 | 0 | 0.297911 | 0.217151 | 0.249518 | 0 | 10 | 546.2114 | 0.732913 |
| 984 | −0.36751 | 0 | 0.304766 | 0.234822 | 0 | 0.26618 | 10 | 544.8497 | 0.737506 |
| 985 | −0.35702 | 0 | 0.341819 | 0 | 0.21209 | 0.213821 | 10 | 545.3195 | 0.733301 |
| 986 | −0.36116 | 0 | 0 | 0.256551 | 0.172681 | 0.188346 | 10 | 542.4325 | 0.740262 |
| 987 | −0.3784 | −0.48038 | 0.429094 | 0.184036 | 0.235336 | 0 | 10 | 532.2363 | 0.754287 |
| 988 | −0.37944 | −0.47686 | 0.432955 | 0.204998 | 0 | 0.247828 | 10 | 532.7806 | 0.75345 |
| 989 | −0.37057 | −0.48826 | 0.469023 | 0 | 0.198485 | 0.194003 | 10 | 532.3628 | 0.752511 |
| 990 | −0.36821 | −0.31823 | 0 | 0.248938 | 0.16128 | 0.17389 | 10 | 536.1157 | 0.751756 |
| 991 | −0.36844 | 0 | 0.31303 | 0.214129 | 0.186465 | 0.210249 | 10 | 546.4186 | 0.733872 |
| 992 | −0.3784 | −0.48038 | 0.429094 | 0.184036 | 0.235336 | 0 | 10 | 528.5449 | 0.760126 |
| 993 | −0.37944 | −0.47686 | 0.432955 | 0.204998 | 0 | 0.247828 | 10 | 527.2524 | 0.763065 |
| 994 | −0.37057 | −0.48826 | 0.469023 | 0 | 0.198485 | 0.194003 | 10 | 527.3306 | 0.760044 |
| 995 | −0.36821 | −0.31823 | 0 | 0.248938 | 0.16128 | 0.17389 | 10 | 529.9912 | 0.761065 |
| 996 | −0.36844 | 0 | 0.31303 | 0.214129 | 0.186465 | 0.210249 | 10 | 541.9956 | 0.739017 |
| 997 | −0.38017 | −0.47149 | 0.43946 | 0.184707 | 0.175726 | 0.194494 | 10 | 529.9913 | 0.757615 |
| 998 | −0.3784 | −0.48038 | 0.429094 | 0.184036 | 0.235336 | 0 | 10 | 529.244 | 0.759411 |
| 999 | −0.37944 | −0.47686 | 0.432955 | 0.204998 | 0 | 0.247828 | 10 | 527.4689 | 0.763188 |
| 1000 | −0.37057 | −0.48826 | 0.469023 | 0 | 0.198485 | 0.194003 | 10 | 527.8663 | 0.759779 |
| 1001 | −0.36821 | −0.31823 | 0 | 0.248938 | 0.16128 | 0.17389 | 10 | 530.3735 | 0.760146 |
| 1002 | −0.36844 | 0 | 0.31303 | 0.214129 | 0.186465 | 0.210249 | 10 | 542.3579 | 0.737812 |
| 1003 | −0.38017 | −0.47149 | 0.43946 | 0.184707 | 0.175726 | 0.194494 | 10 | 531.4706 | 0.754961 |
| 1004 | −0.38017 | −0.47149 | 0.43946 | 0.184707 | 0.175726 | 0.194494 | 10 | 525.241 | 0.764903 |
| 1005 | −0.34604 | 0 | 0 | 0 | 0.20186 | 0.189915 | 10 | 577.2253 | 0.681304 |
| 1006 | −0.36484 | −0.51223 | 0.448932 | 0 | 0 | 0 | 10 | 563.8018 | 0.702148 |
| 1007 | −0.36535 | −0.34122 | 0 | 0.272545 | 0 | 0 | 10 | 574.2639 | 0.685959 |
| 1008 | −0.35247 | −0.33749 | 0 | 0 | 0.244058 | 0 | 10 | 573.6039 | 0.68998 |
| 1009 | −0.3635 | 0 | 0.280897 | 0.247949 | 0 | 0 | 10 | 575.7501 | 0.682059 |
| 1010 | −0.3509 | −0.33426 | 0 | 0 | 0 | 0.230316 | 10 | 573.4853 | 0.684958 |
| 1011 | −0.35435 | 0 | 0.326983 | 0 | 0.276248 | 0 | 10 | 571.8656 | 0.686122 |
| 1012 | −0.35429 | 0 | 0.335115 | 0 | 0 | 0.278056 | 10 | 571.2168 | 0.688817 |
| 1013 | −0.35918 | 0 | 0 | 0.257296 | 0.231108 | 0 | 10 | 576.2697 | 0.683897 |
| 1014 | −0.36046 | 0 | 0 | 0.27483 | 0 | 0.241127 | 10 | 574.9715 | 0.683162 |
| 1015 | −0.37665 | −0.49016 | 0.415897 | 0.213769 | 0 | 0 | 10 | 563.3489 | 0.701127 |
| 1016 | −0.3692 | −0.49878 | 0.458935 | 0 | 0.257995 | 0 | 10 | 559.4615 | 0.707353 |
| 1017 | −0.36789 | −0.4958 | 0.464972 | 0 | 0 | 0.255069 | 10 | 561.0162 | 0.703556 |
| 1018 | −0.36683 | −0.32775 | 0 | 0.247282 | 0.215748 | 0 | 10 | 572.8546 | 0.689041 |
| 1019 | −0.36769 | −0.32552 | 0 | 0.266766 | 0 | 0.223296 | 10 | 573.405 | 0.682386 |
| 1020 | −0.35346 | −0.32601 | 0 | 0 | 0.190682 | 0.171912 | 10 | 575.438 | 0.683631 |
| 1021 | −0.36573 | 0 | 0.297911 | 0.217151 | 0.249518 | 0 | 10 | 571.3598 | 0.691736 |
| 1022 | −0.36751 | 0 | 0.304766 | 0.234822 | 0 | 0.26618 | 10 | 571.8166 | 0.688756 |
| 1023 | −0.35702 | 0 | 0.341819 | 0 | 0.21209 | 0.213821 | 10 | 571.2693 | 0.691716 |
| 1024 | −0.36116 | 0 | 0 | 0.256551 | 0.172681 | 0.188346 | 10 | 577.1882 | 0.679916 |
| 1025 | −0.37665 | −0.49016 | 0.415897 | 0.213769 | 0 | 0 | 10 | 560.7774 | 0.706394 |
| 1026 | −0.3692 | −0.49878 | 0.458935 | 0 | 0.257995 | 0 | 10 | 556.4547 | 0.713274 |
| 1027 | −0.36789 | −0.4958 | 0.464972 | 0 | 0 | 0.255069 | 10 | 556.2019 | 0.711559 |
| 1028 | −0.36683 | −0.32775 | 0 | 0.247282 | 0.215748 | 0 | 10 | 569.3289 | 0.694512 |
| 1029 | −0.36769 | −0.32552 | 0 | 0.266766 | 0 | 0.223296 | 10 | 568.2493 | 0.692716 |
| 1030 | −0.35346 | −0.32601 | 0 | 0 | 0.190682 | 0.171912 | 10 | 570.5658 | 0.690409 |
| 1031 | −0.36573 | 0 | 0.297911 | 0.217151 | 0.249518 | 0 | 10 | 568.9817 | 0.693553 |
| 1032 | −0.36751 | 0 | 0.304766 | 0.234822 | 0 | 0.26618 | 10 | 567.6087 | 0.694329 |
| 1033 | −0.35702 | 0 | 0.341819 | 0 | 0.21209 | 0.213821 | 10 | 567.2163 | 0.693594 |
| 1034 | −0.36116 | 0 | 0 | 0.256551 | 0.172681 | 0.188346 | 10 | 572.656 | 0.685448 |
| 1035 | −0.3784 | −0.48038 | 0.429094 | 0.184036 | 0.235336 | 0 | 10 | 557.5819 | 0.70964 |
| 1036 | −0.37944 | −0.47686 | 0.432955 | 0.204998 | 0 | 0.247828 | 10 | 558.4061 | 0.70717 |
| 1037 | −0.37057 | −0.48826 | 0.469023 | 0 | 0.198485 | 0.194003 | 10 | 557.1772 | 0.708374 |
| 1038 | −0.36821 | −0.31823 | 0 | 0.248938 | 0.16128 | 0.17389 | 10 | 571.3658 | 0.687163 |
| 1039 | −0.36844 | 0 | 0.31303 | 0.214129 | 0.186465 | 0.210249 | 10 | 568.6474 | 0.693941 |
| 1040 | −0.37665 | −0.49016 | 0.415897 | 0.213769 | 0 | 0 | 10 | 560.5974 | 0.707843 |
| 1041 | −0.3692 | −0.49878 | 0.458935 | 0 | 0.257995 | 0 | 10 | 557.0015 | 0.71309 |
| 1042 | −0.36789 | −0.4958 | 0.464972 | 0 | 0 | 0.255069 | 10 | 555.9904 | 0.712498 |
| 1043 | −0.36683 | −0.32775 | 0 | 0.247282 | 0.215748 | 0 | 10 | 569.2639 | 0.694941 |
| 1044 | −0.36769 | −0.32552 | 0 | 0.266766 | 0 | 0.223296 | 10 | 567.6523 | 0.695717 |
| 1045 | −0.35346 | −0.32601 | 0 | 0 | 0.190682 | 0.171912 | 10 | 570.3052 | 0.692716 |
| 1046 | −0.36573 | 0 | 0.297911 | 0.217151 | 0.249518 | 0 | 10 | 569.1485 | 0.691512 |
| 1047 | −0.36751 | 0 | 0.304766 | 0.234822 | 0 | 0.26618 | 10 | 567.0822 | 0.696146 |
| 1048 | −0.35702 | 0 | 0.341819 | 0 | 0.21209 | 0.213821 | 10 | 567.1967 | 0.693104 |
| 1049 | −0.36116 | 0 | 0 | 0.256551 | 0.172681 | 0.188346 | 10 | 572.3262 | 0.686837 |
| 1050 | −0.3784 | −0.48038 | 0.429094 | 0.184036 | 0.235336 | 0 | 10 | 558.6954 | 0.7077 |
| 1051 | −0.37944 | −0.47686 | 0.432955 | 0.204998 | 0 | 0.247828 | 10 | 558.7403 | 0.705802 |
| 1052 | −0.37057 | −0.48826 | 0.469023 | 0 | 0.198485 | 0.194003 | 10 | 558.1941 | 0.706067 |
| 1053 | −0.36821 | −0.31823 | 0 | 0.248938 | 0.16128 | 0.17389 | 10 | 571.6516 | 0.686204 |
| 1054 | −0.36844 | 0 | 0.31303 | 0.214129 | 0.186465 | 0.210249 | 10 | 569.2686 | 0.691042 |
| 1055 | −0.3784 | −0.48038 | 0.429094 | 0.184036 | 0.235336 | 0 | 10 | 555.0508 | 0.714152 |
| 1056 | −0.37944 | −0.47686 | 0.432955 | 0.204998 | 0 | 0.247828 | 10 | 553.3222 | 0.714764 |
| 1057 | −0.37057 | −0.48826 | 0.469023 | 0 | 0.198485 | 0.194003 | 10 | 552.8208 | 0.714621 |
| 1058 | −0.36821 | −0.31823 | 0 | 0.248938 | 0.16128 | 0.17389 | 10 | 566.0106 | 0.695921 |
| 1059 | −0.36844 | 0 | 0.31303 | 0.214129 | 0.186465 | 0.210249 | 10 | 564.4681 | 0.698391 |
| 1060 | −0.38017 | −0.47149 | 0.43946 | 0.184707 | 0.175726 | 0.194494 | 10 | 556.4059 | 0.707435 |
| 1061 | −0.37665 | −0.49016 | 0.415897 | 0.213769 | 0 | 0 | 10 | 561.2531 | 0.706904 |
| 1062 | −0.3692 | −0.49878 | 0.458935 | 0 | 0.257995 | 0 | 10 | 557.1126 | 0.712355 |
| 1063 | −0.36789 | −0.4958 | 0.464972 | 0 | 0 | 0.255069 | 10 | 556.6332 | 0.711293 |
| 1064 | −0.36683 | −0.32775 | 0 | 0.247282 | 0.215748 | 0 | 10 | 569.3258 | 0.697411 |
| 1065 | −0.36769 | −0.32552 | 0 | 0.266766 | 0 | 0.223296 | 10 | 568.0022 | 0.695227 |
| 1066 | −0.35346 | −0.32601 | 0 | 0 | 0.190682 | 0.171912 | 10 | 570.5721 | 0.693328 |
| 1067 | −0.36573 | 0 | 0.297911 | 0.217151 | 0.249518 | 0 | 10 | 569.0009 | 0.692022 |
| 1068 | −0.36751 | 0 | 0.304766 | 0.234822 | 0 | 0.26618 | 10 | 567.3144 | 0.696819 |
| 1069 | −0.35702 | 0 | 0.341819 | 0 | 0.21209 | 0.213821 | 10 | 567.1705 | 0.694451 |
| 1070 | −0.36116 | 0 | 0 | 0.256551 | 0.172681 | 0.188346 | 10 | 572.2214 | 0.687592 |
| 1071 | −0.3784 | −0.48038 | 0.429094 | 0.184036 | 0.235336 | 0 | 10 | 557.5662 | 0.710211 |
| 1072 | −0.37944 | −0.47686 | 0.432955 | 0.204998 | 0 | 0.247828 | 10 | 558.1084 | 0.709619 |
| 1073 | −0.37057 | −0.48826 | 0.469023 | 0 | 0.198485 | 0.194003 | 10 | 557.0503 | 0.709517 |
| 1074 | −0.36821 | −0.31823 | 0 | 0.248938 | 0.16128 | 0.17389 | 10 | 570.6336 | 0.689613 |
| 1075 | −0.36844 | 0 | 0.31303 | 0.214129 | 0.186465 | 0.210249 | 10 | 568.0163 | 0.695043 |
| 1076 | −0.3784 | −0.48038 | 0.429094 | 0.184036 | 0.235336 | 0 | 10 | 554.7897 | 0.71458 |
| 1077 | −0.37944 | −0.47686 | 0.432955 | 0.204998 | 0 | 0.247828 | 10 | 553.4973 | 0.715438 |
| 1078 | −0.37057 | −0.48826 | 0.469023 | 0 | 0.198485 | 0.194003 | 10 | 552.7064 | 0.715213 |
| 1079 | −0.36821 | −0.31823 | 0 | 0.248938 | 0.16128 | 0.17389 | 10 | 565.8121 | 0.698085 |
| 1080 | −0.36844 | 0 | 0.31303 | 0.214129 | 0.186465 | 0.210249 | 10 | 564.0783 | 0.69933 |
| 1081 | −0.38017 | −0.47149 | 0.43946 | 0.184707 | 0.175726 | 0.194494 | 10 | 554.9666 | 0.71213 |
| 1082 | −0.3784 | −0.48038 | 0.429094 | 0.184036 | 0.235336 | 0 | 10 | 555.1703 | 0.714499 |
| 1083 | −0.37944 | −0.47686 | 0.432955 | 0.204998 | 0 | 0.247828 | 10 | 553.3278 | 0.716397 |
| 1084 | −0.37057 | −0.48826 | 0.469023 | 0 | 0.198485 | 0.194003 | 10 | 552.9489 | 0.71556 |
| 1085 | −0.36821 | −0.31823 | 0 | 0.248938 | 0.16128 | 0.17389 | 10 | 565.5668 | 0.699616 |
| 1086 | −0.36844 | 0 | 0.31303 | 0.214129 | 0.186465 | 0.210249 | 10 | 564.0168 | 0.699902 |
| 1087 | −0.38017 | −0.47149 | 0.43946 | 0.184707 | 0.175726 | 0.194494 | 10 | 555.9479 | 0.708742 |
| 1088 | −0.38017 | −0.47149 | 0.43946 | 0.184707 | 0.175726 | 0.194494 | 10 | 550.6796 | 0.717214 |
| 1089 | −0.37665 | −0.49016 | 0.415897 | 0.213769 | 0 | 0 | 10 | 562.5512 | 0.708885 |
| 1090 | −0.3692 | −0.49878 | 0.458935 | 0 | 0.257995 | 0 | 10 | 558.9431 | 0.712273 |
| 1091 | −0.36789 | −0.4958 | 0.464972 | 0 | 0 | 0.255069 | 10 | 558.2807 | 0.711293 |
| 1092 | −0.36683 | −0.32775 | 0 | 0.247282 | 0.215748 | 0 | 10 | 570.18 | 0.697105 |
| 1093 | −0.36769 | −0.32552 | 0 | 0.266766 | 0 | 0.223296 | 10 | 568.7978 | 0.694798 |
| 1094 | −0.35346 | −0.32601 | 0 | 0 | 0.190682 | 0.171912 | 10 | 571.4504 | 0.692675 |
| 1095 | −0.36573 | 0 | 0.297911 | 0.217151 | 0.249518 | 0 | 10 | 570.7949 | 0.691369 |
| 1096 | −0.36751 | 0 | 0.304766 | 0.234822 | 0 | 0.26618 | 10 | 569.0177 | 0.693757 |
| 1097 | −0.35702 | 0 | 0.341819 | 0 | 0.21209 | 0.213821 | 10 | 569.2366 | 0.691859 |
| 1098 | −0.36116 | 0 | 0 | 0.256551 | 0.172681 | 0.188346 | 10 | 573.4085 | 0.685714 |
| 1099 | −0.3784 | −0.48038 | 0.429094 | 0.184036 | 0.235336 | 0 | 10 | 559.5378 | 0.709579 |
| 1100 | −0.37944 | −0.47686 | 0.432955 | 0.204998 | 0 | 0.247828 | 10 | 559.8458 | 0.706639 |
| 1101 | −0.37057 | −0.48826 | 0.469023 | 0 | 0.198485 | 0.194003 | 10 | 559.2049 | 0.707762 |
| 1102 | −0.36821 | −0.31823 | 0 | 0.248938 | 0.16128 | 0.17389 | 10 | 571.744 | 0.68994 |
| 1103 | −0.36844 | 0 | 0.31303 | 0.214129 | 0.186465 | 0.210249 | 10 | 570.0926 | 0.692022 |
| 1104 | −0.3784 | −0.48038 | 0.429094 | 0.184036 | 0.235336 | 0 | 10 | 556.4796 | 0.717234 |
| 1105 | −0.37944 | −0.47686 | 0.432955 | 0.204998 | 0 | 0.247828 | 10 | 555.0317 | 0.716683 |
| 1106 | −0.37057 | −0.48826 | 0.469023 | 0 | 0.198485 | 0.194003 | 10 | 554.619 | 0.715376 |
| 1107 | −0.36821 | −0.31823 | 0 | 0.248938 | 0.16128 | 0.17389 | 10 | 566.6703 | 0.698208 |
| 1108 | −0.36844 | 0 | 0.31303 | 0.214129 | 0.186465 | 0.210249 | 10 | 565.9012 | 0.697636 |
| 1109 | −0.38017 | −0.47149 | 0.43946 | 0.184707 | 0.175726 | 0.194494 | 10 | 556.8375 | 0.710048 |
| 1110 | −0.3784 | −0.48038 | 0.429094 | 0.184036 | 0.235336 | 0 | 10 | 557.4848 | 0.715111 |
| 1111 | −0.37944 | −0.47686 | 0.432955 | 0.204998 | 0 | 0.247828 | 10 | 555.315 | 0.716561 |
| 1112 | −0.37057 | −0.48826 | 0.469023 | 0 | 0.198485 | 0.194003 | 10 | 555.4864 | 0.713355 |
| 1113 | −0.36821 | −0.31823 | 0 | 0.248938 | 0.16128 | 0.17389 | 10 | 566.9411 | 0.699902 |
| 1114 | −0.36844 | 0 | 0.31303 | 0.214129 | 0.186465 | 0.210249 | 10 | 566.4226 | 0.695125 |
| 1115 | −0.38017 | −0.47149 | 0.43946 | 0.184707 | 0.175726 | 0.194494 | 10 | 558.3846 | 0.707006 |
| 1116 | −0.38017 | −0.47149 | 0.43946 | 0.184707 | 0.175726 | 0.194494 | 10 | 552.9774 | 0.716438 |
| 1117 | −0.34954 | 0 | 0.311734 | 0 | 0 | 0 | 10 | 510.2992 | 0.782296 |
| 1118 | −0.34838 | −0.35299 | 0 | 0 | 0 | 0 | 10 | 506.5011 | 0.785542 |
| 1119 | −0.35731 | 0 | 0 | 0.283735 | 0 | 0 | 10 | 510.5404 | 0.78001 |
| 1120 | −0.34421 | 0 | 0 | 0 | 0.260301 | 0 | 10 | 510.2552 | 0.781316 |
| 1121 | −0.34349 | 0 | 0 | 0 | 0 | 0.25144 | 10 | 510.3644 | 0.782255 |
| 1122 | −0.34954 | 0 | 0.311734 | 0 | 0 | 0 | 10 | 507.4947 | 0.786747 |
| 1123 | −0.34838 | −0.35299 | 0 | 0 | 0 | 0 | 10 | 502.2091 | 0.791769 |
| 1124 | −0.35731 | 0 | 0 | 0.283735 | 0 | 0 | 10 | 507.0536 | 0.786175 |
| 1125 | −0.34421 | 0 | 0 | 0 | 0.260301 | 0 | 10 | 506.5899 | 0.787196 |
| 1126 | −0.34349 | 0 | 0 | 0 | 0 | 0.25144 | 10 | 505.6958 | 0.789441 |
| 1127 | −0.34604 | 0 | 0 | 0 | 0.20186 | 0.189915 | 10 | 509.1378 | 0.783419 |
| 1128 | −0.36484 | −0.51223 | 0.448932 | 0 | 0 | 0 | 10 | 498.6007 | 0.795076 |
| 1129 | −0.36535 | −0.34122 | 0 | 0.272545 | 0 | 0 | 10 | 504.0565 | 0.787461 |
| 1130 | −0.3635 | 0 | 0.280897 | 0.247949 | 0 | 0 | 10 | 508.3814 | 0.784317 |
| 1131 | −0.35247 | −0.33749 | 0 | 0 | 0.244058 | 0 | 10 | 503.8807 | 0.787257 |
| 1132 | −0.3509 | −0.33426 | 0 | 0 | 0 | 0.230316 | 10 | 504.2711 | 0.788686 |
| 1133 | −0.35435 | 0 | 0.326983 | 0 | 0.276248 | 0 | 10 | 506.9982 | 0.785767 |
| 1134 | −0.35429 | 0 | 0.335115 | 0 | 0 | 0.278056 | 10 | 507.0363 | 0.786379 |
| 1135 | −0.35918 | 0 | 0 | 0.257296 | 0.231108 | 0 | 10 | 508.2912 | 0.783766 |
| 1136 | −0.36046 | 0 | 0 | 0.27483 | 0 | 0.241127 | 10 | 508.0047 | 0.784562 |
| 1137 | −0.34954 | 0 | 0.311734 | 0 | 0 | 0 | 10 | 507.4927 | 0.785971 |
| 1138 | −0.34838 | −0.35299 | 0 | 0 | 0 | 0 | 10 | 502.0968 | 0.792197 |
| 1139 | −0.35731 | 0 | 0 | 0.283735 | 0 | 0 | 10 | 506.9753 | 0.785848 |
| 1140 | −0.34421 | 0 | 0 | 0 | 0.260301 | 0 | 10 | 506.8664 | 0.785379 |
| 1141 | −0.34349 | 0 | 0 | 0 | 0 | 0.25144 | 10 | 505.4897 | 0.789054 |
| 1142 | −0.34604 | 0 | 0 | 0 | 0.20186 | 0.189915 | 10 | 510.133 | 0.781112 |
| 1143 | −0.36484 | −0.51223 | 0.448932 | 0 | 0 | 0 | 10 | 499.5173 | 0.794545 |
| 1144 | −0.36535 | −0.34122 | 0 | 0.272545 | 0 | 0 | 10 | 504.703 | 0.78644 |
| 1145 | −0.3635 | 0 | 0.280897 | 0.247949 | 0 | 0 | 10 | 509.1118 | 0.782153 |
| 1146 | −0.35247 | −0.33749 | 0 | 0 | 0.244058 | 0 | 10 | 505.0917 | 0.786318 |
| 1147 | −0.3509 | −0.33426 | 0 | 0 | 0 | 0.230316 | 10 | 504.8426 | 0.788298 |
| 1148 | −0.35435 | 0 | 0.326983 | 0 | 0.276248 | 0 | 10 | 508.4231 | 0.783582 |
| 1149 | −0.35429 | 0 | 0.335115 | 0 | 0 | 0.278056 | 10 | 507.7901 | 0.785011 |
| 1150 | −0.35918 | 0 | 0 | 0.257296 | 0.231108 | 0 | 10 | 509.453 | 0.779867 |
| 1151 | −0.36046 | 0 | 0 | 0.27483 | 0 | 0.241127 | 10 | 508.65 | 0.78248 |
| 1152 | −0.34604 | 0 | 0 | 0 | 0.20186 | 0.189915 | 10 | 504.6124 | 0.788645 |
| 1153 | −0.36484 | −0.51223 | 0.448932 | 0 | 0 | 0 | 10 | 495.0154 | 0.801874 |
| 1154 | −0.36535 | −0.34122 | 0 | 0.272545 | 0 | 0 | 10 | 499.471 | 0.794014 |
| 1155 | −0.3635 | 0 | 0.280897 | 0.247949 | 0 | 0 | 10 | 505.5119 | 0.787482 |
| 1156 | −0.35247 | −0.33749 | 0 | 0 | 0.244058 | 0 | 10 | 499.5103 | 0.79332 |
| 1157 | −0.3509 | −0.33426 | 0 | 0 | 0 | 0.230316 | 10 | 498.1355 | 0.796668 |
| 1158 | −0.35435 | 0 | 0.326983 | 0 | 0.276248 | 0 | 10 | 504.4766 | 0.788849 |
| 1159 | −0.35429 | 0 | 0.335115 | 0 | 0 | 0.278056 | 10 | 502.841 | 0.791034 |
| 1160 | −0.35918 | 0 | 0 | 0.257296 | 0.231108 | 0 | 10 | 504.8077 | 0.787339 |
| 1161 | −0.36046 | 0 | 0 | 0.27483 | 0 | 0.241127 | 10 | 502.9596 | 0.790605 |
| 1162 | −0.37665 | −0.49016 | 0.415897 | 0.213769 | 0 | 0 | 10 | 498.2274 | 0.796015 |
| 1163 | −0.3692 | −0.49878 | 0.458935 | 0 | 0.257995 | 0 | 10 | 497.3042 | 0.79577 |
| 1164 | −0.36789 | −0.4958 | 0.464972 | 0 | 0 | 0.255069 | 10 | 496.9034 | 0.797444 |
| 1165 | −0.36683 | −0.32775 | 0 | 0.247282 | 0.215748 | 0 | 10 | 503.3268 | 0.787318 |
| 1166 | −0.36769 | −0.32552 | 0 | 0.266766 | 0 | 0.223296 | 10 | 502.6989 | 0.790258 |
| 1167 | −0.35346 | −0.32601 | 0 | 0 | 0.190682 | 0.171912 | 10 | 504.2318 | 0.787686 |
| 1168 | −0.36573 | 0 | 0.297911 | 0.217151 | 0.249518 | 0 | 10 | 507.1486 | 0.783929 |
| 1169 | −0.36751 | 0 | 0.304766 | 0.234822 | 0 | 0.26618 | 10 | 506.2148 | 0.786175 |
| 1170 | −0.35702 | 0 | 0.341819 | 0 | 0.21209 | 0.213821 | 10 | 506.8683 | 0.784583 |
| 1171 | −0.36116 | 0 | 0 | 0.256551 | 0.172681 | 0.188346 | 10 | 508.2971 | 0.78197 |
| 1172 | −0.34954 | 0 | 0.311734 | 0 | 0 | 0 | 10 | 506.9476 | 0.788257 |
| 1173 | −0.34838 | −0.35299 | 0 | 0 | 0 | 0 | 10 | 501.7931 | 0.792279 |
| 1174 | −0.35731 | 0 | 0 | 0.283735 | 0 | 0 | 10 | 506.4242 | 0.786788 |
| 1175 | −0.34421 | 0 | 0 | 0 | 0.260301 | 0 | 10 | 505.9987 | 0.787972 |
| 1176 | −0.34349 | 0 | 0 | 0 | 0 | 0.25144 | 10 | 504.9886 | 0.790442 |
| 1177 | −0.34604 | 0 | 0 | 0 | 0.20186 | 0.189915 | 10 | 508.35 | 0.783501 |
| 1178 | −0.36484 | −0.51223 | 0.448932 | 0 | 0 | 0 | 10 | 498.1566 | 0.795076 |
| 1179 | −0.36535 | −0.34122 | 0 | 0.272545 | 0 | 0 | 10 | 503.4197 | 0.788176 |
| 1180 | −0.3635 | 0 | 0.280897 | 0.247949 | 0 | 0 | 10 | 507.727 | 0.784562 |
| 1181 | −0.35247 | −0.33749 | 0 | 0 | 0.244058 | 0 | 10 | 503.2477 | 0.788196 |
| 1182 | −0.3509 | −0.33426 | 0 | 0 | 0 | 0.230316 | 10 | 503.5526 | 0.78938 |
| 1183 | −0.35435 | 0 | 0.326983 | 0 | 0.276248 | 0 | 10 | 506.3613 | 0.785828 |
| 1184 | −0.35429 | 0 | 0.335115 | 0 | 0 | 0.278056 | 10 | 506.3352 | 0.786788 |
| 1185 | −0.35918 | 0 | 0 | 0.257296 | 0.231108 | 0 | 10 | 507.5811 | 0.783684 |
| 1186 | −0.36046 | 0 | 0 | 0.27483 | 0 | 0.241127 | 10 | 507.2593 | 0.78446 |
| 1187 | −0.34604 | 0 | 0 | 0 | 0.20186 | 0.189915 | 10 | 503.6613 | 0.790258 |
| 1188 | −0.36484 | −0.51223 | 0.448932 | 0 | 0 | 0 | 10 | 494.4066 | 0.801894 |
| 1189 | −0.36535 | −0.34122 | 0 | 0.272545 | 0 | 0 | 10 | 498.9017 | 0.794811 |
| 1190 | −0.3635 | 0 | 0.280897 | 0.247949 | 0 | 0 | 10 | 504.7621 | 0.788829 |
| 1191 | −0.35247 | −0.33749 | 0 | 0 | 0.244058 | 0 | 10 | 498.597 | 0.795056 |
| 1192 | −0.3509 | −0.33426 | 0 | 0 | 0 | 0.230316 | 10 | 497.6337 | 0.797015 |
| 1193 | −0.35435 | 0 | 0.326983 | 0 | 0.276248 | 0 | 10 | 503.2641 | 0.791401 |
| 1194 | −0.35429 | 0 | 0.335115 | 0 | 0 | 0.278056 | 10 | 502.1047 | 0.792157 |
| 1195 | −0.35918 | 0 | 0 | 0.257296 | 0.231108 | 0 | 10 | 503.7421 | 0.788482 |
| 1196 | −0.36046 | 0 | 0 | 0.27483 | 0 | 0.241127 | 10 | 502.2727 | 0.79132 |
| 1197 | −0.37665 | −0.49016 | 0.415897 | 0.213769 | 0 | 0 | 10 | 496.6246 | 0.796199 |
| 1198 | −0.3692 | −0.49878 | 0.458935 | 0 | 0.257995 | 0 | 10 | 494.9142 | 0.798669 |
| 1199 | −0.36789 | −0.4958 | 0.464972 | 0 | 0 | 0.255069 | 10 | 495.2362 | 0.798751 |
| 1200 | −0.36683 | −0.32775 | 0 | 0.247282 | 0.215748 | 0 | 10 | 501.3016 | 0.789809 |
| 1201 | −0.36769 | −0.32552 | 0 | 0.266766 | 0 | 0.223296 | 10 | 501.2073 | 0.791115 |
| 1202 | −0.35346 | −0.32601 | 0 | 0 | 0.190682 | 0.171912 | 10 | 502.322 | 0.790095 |
| 1203 | −0.36573 | 0 | 0.297911 | 0.217151 | 0.249518 | 0 | 10 | 504.953 | 0.787461 |
| 1204 | −0.36751 | 0 | 0.304766 | 0.234822 | 0 | 0.26618 | 10 | 504.6295 | 0.787808 |
| 1205 | −0.35702 | 0 | 0.341819 | 0 | 0.21209 | 0.213821 | 10 | 504.7479 | 0.788074 |
| 1206 | −0.36116 | 0 | 0 | 0.256551 | 0.172681 | 0.188346 | 10 | 506.406 | 0.785358 |
| 1207 | −0.34604 | 0 | 0 | 0 | 0.20186 | 0.189915 | 10 | 503.8452 | 0.789707 |
| 1208 | −0.36484 | −0.51223 | 0.448932 | 0 | 0 | 0 | 10 | 494.6404 | 0.802078 |
| 1209 | −0.36535 | −0.34122 | 0 | 0.272545 | 0 | 0 | 10 | 498.9259 | 0.794749 |
| 1210 | −0.3635 | 0 | 0.280897 | 0.247949 | 0 | 0 | 10 | 504.8633 | 0.788257 |
| 1211 | −0.35247 | −0.33749 | 0 | 0 | 0.244058 | 0 | 10 | 498.9826 | 0.793239 |
| 1212 | −0.3509 | −0.33426 | 0 | 0 | 0 | 0.230316 | 10 | 497.5206 | 0.797281 |
| 1213 | −0.35435 | 0 | 0.326983 | 0 | 0.276248 | 0 | 10 | 503.8277 | 0.789768 |
| 1214 | −0.35429 | 0 | 0.335115 | 0 | 0 | 0.278056 | 10 | 502.1262 | 0.792157 |
| 1215 | −0.35918 | 0 | 0 | 0.257296 | 0.231108 | 0 | 10 | 504.1083 | 0.788257 |
| 1216 | −0.36046 | 0 | 0 | 0.27483 | 0 | 0.241127 | 10 | 502.2071 | 0.791728 |
| 1217 | −0.37665 | −0.49016 | 0.415897 | 0.213769 | 0 | 0 | 10 | 497.7234 | 0.79626 |
| 1218 | −0.3692 | −0.49878 | 0.458935 | 0 | 0.257995 | 0 | 10 | 496.8252 | 0.795831 |
| 1219 | −0.36789 | −0.4958 | 0.464972 | 0 | 0 | 0.255069 | 10 | 496.3903 | 0.797852 |
| 1220 | −0.36683 | −0.32775 | 0 | 0.247282 | 0.215748 | 0 | 10 | 502.7271 | 0.78791 |
| 1221 | −0.36769 | −0.32552 | 0 | 0.266766 | 0 | 0.223296 | 10 | 502.0952 | 0.79132 |
| 1222 | −0.35346 | −0.32601 | 0 | 0 | 0.190682 | 0.171912 | 10 | 503.5824 | 0.788441 |
| 1223 | −0.36573 | 0 | 0.297911 | 0.217151 | 0.249518 | 0 | 10 | 506.5638 | 0.784889 |
| 1224 | −0.36751 | 0 | 0.304766 | 0.234822 | 0 | 0.26618 | 10 | 505.6461 | 0.785951 |
| 1225 | −0.35702 | 0 | 0.341819 | 0 | 0.21209 | 0.213821 | 10 | 506.2655 | 0.785971 |
| 1226 | −0.36116 | 0 | 0 | 0.256551 | 0.172681 | 0.188346 | 10 | 507.6758 | 0.783011 |
| 1227 | −0.37665 | −0.49016 | 0.415897 | 0.213769 | 0 | 0 | 10 | 492.9723 | 0.802732 |
| 1228 | −0.3692 | −0.49878 | 0.458935 | 0 | 0.257995 | 0 | 10 | 491.6043 | 0.803324 |
| 1229 | −0.36789 | −0.4958 | 0.464972 | 0 | 0 | 0.255069 | 10 | 489.9297 | 0.80559 |
| 1230 | −0.36683 | −0.32775 | 0 | 0.247282 | 0.215748 | 0 | 10 | 496.8055 | 0.796035 |
| 1231 | −0.36769 | −0.32552 | 0 | 0.266766 | 0 | 0.223296 | 10 | 494.8689 | 0.7992 |
| 1232 | −0.35346 | −0.32601 | 0 | 0 | 0.190682 | 0.171912 | 10 | 496.6498 | 0.796648 |
| 1233 | −0.36573 | 0 | 0.297911 | 0.217151 | 0.249518 | 0 | 10 | 502.3256 | 0.791177 |
| 1234 | −0.36751 | 0 | 0.304766 | 0.234822 | 0 | 0.26618 | 10 | 500.2768 | 0.793279 |
| 1235 | −0.35702 | 0 | 0.341819 | 0 | 0.21209 | 0.213821 | 10 | 501.0123 | 0.79232 |
| 1236 | −0.36116 | 0 | 0 | 0.256551 | 0.172681 | 0.188346 | 10 | 501.6553 | 0.791463 |
| 1237 | −0.3784 | −0.48038 | 0.429094 | 0.184036 | 0.235336 | 0 | 10 | 496.0057 | 0.797281 |
| 1238 | −0.37944 | −0.47686 | 0.432955 | 0.204998 | 0 | 0.247828 | 10 | 495.3408 | 0.799751 |
| 1239 | −0.37057 | −0.48826 | 0.469023 | 0 | 0.198485 | 0.194003 | 10 | 495.6036 | 0.798199 |
| 1240 | −0.36821 | −0.31823 | 0 | 0.248938 | 0.16128 | 0.17389 | 10 | 501.9158 | 0.790646 |
| 1241 | −0.36844 | 0 | 0.31303 | 0.214129 | 0.186465 | 0.210249 | 10 | 505.1802 | 0.786747 |
| 1242 | −0.34954 | 0 | 0.311734 | 0 | 0 | 0 | 10 | 510.4961 | 0.784828 |
| 1243 | −0.34838 | −0.35299 | 0 | 0 | 0 | 0 | 10 | 505.3043 | 0.787584 |
| 1244 | −0.35731 | 0 | 0 | 0.283735 | 0 | 0 | 10 | 509.568 | 0.783317 |
| 1245 | −0.34421 | 0 | 0 | 0 | 0.260301 | 0 | 10 | 509.4696 | 0.784195 |
| 1246 | −0.34349 | 0 | 0 | 0 | 0 | 0.25144 | 10 | 508.4485 | 0.784664 |
| 1247 | −0.34604 | 0 | 0 | 0 | 0.20186 | 0.189915 | 10 | 511.4179 | 0.780745 |
| 1248 | −0.36484 | −0.51223 | 0.448932 | 0 | 0 | 0 | 10 | 502.0843 | 0.791095 |
| 1249 | −0.36535 | −0.34122 | 0 | 0.272545 | 0 | 0 | 10 | 506.4327 | 0.784032 |
| 1250 | −0.3635 | 0 | 0.280897 | 0.247949 | 0 | 0 | 10 | 510.8747 | 0.779989 |
| 1251 | −0.35247 | −0.33749 | 0 | 0 | 0.244058 | 0 | 10 | 506.5859 | 0.783848 |
| 1252 | −0.3509 | −0.33426 | 0 | 0 | 0 | 0.230316 | 10 | 506.694 | 0.784256 |
| 1253 | −0.35435 | 0 | 0.326983 | 0 | 0.276248 | 0 | 10 | 509.9736 | 0.782725 |
| 1254 | −0.35429 | 0 | 0.335115 | 0 | 0 | 0.278056 | 10 | 509.7136 | 0.782521 |
| 1255 | −0.35918 | 0 | 0 | 0.257296 | 0.231108 | 0 | 10 | 510.5439 | 0.781194 |
| 1256 | −0.36046 | 0 | 0 | 0.27483 | 0 | 0.241127 | 10 | 510.028 | 0.781031 |
| 1257 | −0.34604 | 0 | 0 | 0 | 0.20186 | 0.189915 | 10 | 507.0689 | 0.786032 |
| 1258 | −0.36484 | −0.51223 | 0.448932 | 0 | 0 | 0 | 10 | 498.6138 | 0.797158 |
| 1259 | −0.36535 | −0.34122 | 0 | 0.272545 | 0 | 0 | 10 | 502.1959 | 0.78985 |
| 1260 | −0.3635 | 0 | 0.280897 | 0.247949 | 0 | 0 | 10 | 508.0331 | 0.785114 |
| 1261 | −0.35247 | −0.33749 | 0 | 0 | 0.244058 | 0 | 10 | 502.2433 | 0.789584 |
| 1262 | −0.3509 | −0.33426 | 0 | 0 | 0 | 0.230316 | 10 | 501.262 | 0.791871 |
| 1263 | −0.35435 | 0 | 0.326983 | 0 | 0.276248 | 0 | 10 | 507.0327 | 0.786216 |
| 1264 | −0.35429 | 0 | 0.335115 | 0 | 0 | 0.278056 | 10 | 505.8156 | 0.787094 |
| 1265 | −0.35918 | 0 | 0 | 0.257296 | 0.231108 | 0 | 10 | 506.9202 | 0.786032 |
| 1266 | −0.36046 | 0 | 0 | 0.27483 | 0 | 0.241127 | 10 | 505.4022 | 0.786726 |
| 1267 | −0.37665 | −0.49016 | 0.415897 | 0.213769 | 0 | 0 | 10 | 500.2062 | 0.792381 |
| 1268 | −0.3692 | −0.49878 | 0.458935 | 0 | 0.257995 | 0 | 10 | 498.9933 | 0.794382 |
| 1269 | −0.36789 | −0.4958 | 0.464972 | 0 | 0 | 0.255069 | 10 | 499.0251 | 0.794647 |
| 1270 | −0.36683 | −0.32775 | 0 | 0.247282 | 0.215748 | 0 | 10 | 504.2501 | 0.786379 |
| 1271 | −0.36769 | −0.32552 | 0 | 0.266766 | 0 | 0.223296 | 10 | 503.9197 | 0.787216 |
| 1272 | −0.35346 | −0.32601 | 0 | 0 | 0.190682 | 0.171912 | 10 | 505.3658 | 0.786685 |
| 1273 | −0.36573 | 0 | 0.297911 | 0.217151 | 0.249518 | 0 | 10 | 508.1329 | 0.78444 |
| 1274 | −0.36751 | 0 | 0.304766 | 0.234822 | 0 | 0.26618 | 10 | 507.5408 | 0.784134 |
| 1275 | −0.35702 | 0 | 0.341819 | 0 | 0.21209 | 0.213821 | 10 | 508.0834 | 0.784154 |
| 1276 | −0.36116 | 0 | 0 | 0.256551 | 0.172681 | 0.188346 | 10 | 509.0051 | 0.782888 |
| 1277 | −0.34604 | 0 | 0 | 0 | 0.20186 | 0.189915 | 10 | 507.8393 | 0.784358 |
| 1278 | −0.36484 | −0.51223 | 0.448932 | 0 | 0 | 0 | 10 | 499.3515 | 0.796587 |
| 1279 | −0.36535 | −0.34122 | 0 | 0.272545 | 0 | 0 | 10 | 502.7037 | 0.788747 |
| 1280 | −0.3635 | 0 | 0.280897 | 0.247949 | 0 | 0 | 10 | 508.6613 | 0.783582 |
| 1281 | −0.35247 | −0.33749 | 0 | 0 | 0.244058 | 0 | 10 | 503.1978 | 0.789237 |
| 1282 | −0.3509 | −0.33426 | 0 | 0 | 0 | 0.230316 | 10 | 501.647 | 0.791605 |
| 1283 | −0.35435 | 0 | 0.326983 | 0 | 0.276248 | 0 | 10 | 508.1957 | 0.783929 |
| 1284 | −0.35429 | 0 | 0.335115 | 0 | 0 | 0.278056 | 10 | 506.391 | 0.785991 |
| 1285 | −0.35918 | 0 | 0 | 0.257296 | 0.231108 | 0 | 10 | 507.8622 | 0.783399 |
| 1286 | −0.36046 | 0 | 0 | 0.27483 | 0 | 0.241127 | 10 | 505.8655 | 0.785236 |
| 1287 | −0.37665 | −0.49016 | 0.415897 | 0.213769 | 0 | 0 | 10 | 501.767 | 0.79087 |
| 1288 | −0.3692 | −0.49878 | 0.458935 | 0 | 0.257995 | 0 | 10 | 501.3828 | 0.791177 |
| 1289 | −0.36789 | −0.4958 | 0.464972 | 0 | 0 | 0.255069 | 10 | 500.6371 | 0.792545 |
| 1290 | −0.36683 | −0.32775 | 0 | 0.247282 | 0.215748 | 0 | 10 | 506.135 | 0.783746 |
| 1291 | −0.36769 | −0.32552 | 0 | 0.266766 | 0 | 0.223296 | 10 | 505.2381 | 0.78544 |
| 1292 | −0.35346 | −0.32601 | 0 | 0 | 0.190682 | 0.171912 | 10 | 507.0865 | 0.783787 |
| 1293 | −0.36573 | 0 | 0.297911 | 0.217151 | 0.249518 | 0 | 10 | 510.2064 | 0.781031 |
| 1294 | −0.36751 | 0 | 0.304766 | 0.234822 | 0 | 0.26618 | 10 | 508.9963 | 0.781337 |
| 1295 | −0.35702 | 0 | 0.341819 | 0 | 0.21209 | 0.213821 | 10 | 510.0532 | 0.7805 |
| 1296 | −0.36116 | 0 | 0 | 0.256551 | 0.172681 | 0.188346 | 10 | 510.7097 | 0.778785 |
| 1297 | −0.37665 | −0.49016 | 0.415897 | 0.213769 | 0 | 0 | 10 | 497.3675 | 0.797771 |
| 1298 | −0.3692 | −0.49878 | 0.458935 | 0 | 0.257995 | 0 | 10 | 496.6377 | 0.797771 |
| 1299 | −0.36789 | −0.4958 | 0.464972 | 0 | 0 | 0.255069 | 10 | 494.8242 | 0.799914 |
| 1300 | −0.36683 | −0.32775 | 0 | 0.247282 | 0.215748 | 0 | 10 | 500.6807 | 0.792034 |
| 1301 | −0.36769 | −0.32552 | 0 | 0.266766 | 0 | 0.223296 | 10 | 498.6395 | 0.794035 |
| 1302 | −0.35346 | −0.32601 | 0 | 0 | 0.190682 | 0.171912 | 10 | 500.7787 | 0.792116 |
| 1303 | −0.36573 | 0 | 0.297911 | 0.217151 | 0.249518 | 0 | 10 | 506.2631 | 0.785869 |
| 1304 | −0.36751 | 0 | 0.304766 | 0.234822 | 0 | 0.26618 | 10 | 504.0767 | 0.788237 |
| 1305 | −0.35702 | 0 | 0.341819 | 0 | 0.21209 | 0.213821 | 10 | 505.2849 | 0.787277 |
| 1306 | −0.36116 | 0 | 0 | 0.256551 | 0.172681 | 0.188346 | 10 | 505.2066 | 0.786134 |
| 1307 | −0.3784 | −0.48038 | 0.429094 | 0.184036 | 0.235336 | 0 | 10 | 500.0344 | 0.793055 |
| 1308 | −0.37944 | −0.47686 | 0.432955 | 0.204998 | 0 | 0.247828 | 10 | 499.0235 | 0.794974 |
| 1309 | −0.37057 | −0.48826 | 0.469023 | 0 | 0.198485 | 0.194003 | 10 | 499.7547 | 0.792973 |
| 1310 | −0.36821 | −0.31823 | 0 | 0.248938 | 0.16128 | 0.17389 | 10 | 504.8097 | 0.785828 |
| 1311 | −0.36844 | 0 | 0.31303 | 0.214129 | 0.186465 | 0.210249 | 10 | 508.3129 | 0.782725 |
| 1312 | −0.34604 | 0 | 0 | 0 | 0.20186 | 0.189915 | 10 | 506.8315 | 0.786808 |
| 1313 | −0.36484 | −0.51223 | 0.448932 | 0 | 0 | 0 | 10 | 498.7685 | 0.79722 |
| 1314 | −0.36535 | −0.34122 | 0 | 0.272545 | 0 | 0 | 10 | 502.1483 | 0.788727 |
| 1315 | −0.3635 | 0 | 0.280897 | 0.247949 | 0 | 0 | 10 | 507.8759 | 0.785624 |
| 1316 | −0.35247 | −0.33749 | 0 | 0 | 0.244058 | 0 | 10 | 502.2865 | 0.790074 |
| 1317 | −0.3509 | −0.33426 | 0 | 0 | 0 | 0.230316 | 10 | 501.1557 | 0.792545 |
| 1318 | −0.35435 | 0 | 0.326983 | 0 | 0.276248 | 0 | 10 | 506.9406 | 0.786869 |
| 1319 | −0.35429 | 0 | 0.335115 | 0 | 0 | 0.278056 | 10 | 505.5945 | 0.787767 |
| 1320 | −0.35918 | 0 | 0 | 0.257296 | 0.231108 | 0 | 10 | 506.7335 | 0.785685 |
| 1321 | −0.36046 | 0 | 0 | 0.27483 | 0 | 0.241127 | 10 | 505.0987 | 0.78738 |
| 1322 | −0.37665 | −0.49016 | 0.415897 | 0.213769 | 0 | 0 | 10 | 500.0435 | 0.792442 |
| 1323 | −0.3692 | −0.49878 | 0.458935 | 0 | 0.257995 | 0 | 10 | 498.8626 | 0.794484 |
| 1324 | −0.36789 | −0.4958 | 0.464972 | 0 | 0 | 0.255069 | 10 | 498.7828 | 0.794443 |
| 1325 | −0.36683 | −0.32775 | 0 | 0.247282 | 0.215748 | 0 | 10 | 503.9373 | 0.786808 |
| 1326 | −0.36769 | −0.32552 | 0 | 0.266766 | 0 | 0.223296 | 10 | 503.5288 | 0.786767 |
| 1327 | −0.35346 | −0.32601 | 0 | 0 | 0.190682 | 0.171912 | 10 | 504.9716 | 0.786706 |
| 1328 | −0.36573 | 0 | 0.297911 | 0.217151 | 0.249518 | 0 | 10 | 507.8234 | 0.784787 |
| 1329 | −0.36751 | 0 | 0.304766 | 0.234822 | 0 | 0.26618 | 10 | 507.1631 | 0.783378 |
| 1330 | −0.35702 | 0 | 0.341819 | 0 | 0.21209 | 0.213821 | 10 | 507.7122 | 0.784481 |
| 1331 | −0.36116 | 0 | 0 | 0.256551 | 0.172681 | 0.188346 | 10 | 508.5619 | 0.782664 |
| 1332 | −0.37665 | −0.49016 | 0.415897 | 0.213769 | 0 | 0 | 10 | 496.536 | 0.797893 |
| 1333 | −0.3692 | −0.49878 | 0.458935 | 0 | 0.257995 | 0 | 10 | 495.2688 | 0.799669 |
| 1334 | −0.36789 | −0.4958 | 0.464972 | 0 | 0 | 0.255069 | 10 | 493.9837 | 0.80069 |
| 1335 | −0.36683 | −0.32775 | 0 | 0.247282 | 0.215748 | 0 | 10 | 499.5448 | 0.792912 |
| 1336 | −0.36769 | −0.32552 | 0 | 0.266766 | 0 | 0.223296 | 10 | 497.8794 | 0.794178 |
| 1337 | −0.35346 | −0.32601 | 0 | 0 | 0.190682 | 0.171912 | 10 | 499.7823 | 0.792932 |
| 1338 | −0.36573 | 0 | 0.297911 | 0.217151 | 0.249518 | 0 | 10 | 504.8362 | 0.787543 |
| 1339 | −0.36751 | 0 | 0.304766 | 0.234822 | 0 | 0.26618 | 10 | 503.0959 | 0.789993 |
| 1340 | −0.35702 | 0 | 0.341819 | 0 | 0.21209 | 0.213821 | 10 | 503.9481 | 0.788972 |
| 1341 | −0.36116 | 0 | 0 | 0.256551 | 0.172681 | 0.188346 | 10 | 504.0193 | 0.787992 |
| 1342 | −0.3784 | −0.48038 | 0.429094 | 0.184036 | 0.235336 | 0 | 10 | 497.3679 | 0.79624 |
| 1343 | −0.37944 | −0.47686 | 0.432955 | 0.204998 | 0 | 0.247828 | 10 | 496.9752 | 0.795484 |
| 1344 | −0.37057 | −0.48826 | 0.469023 | 0 | 0.198485 | 0.194003 | 10 | 497.1426 | 0.796219 |
| 1345 | −0.36821 | −0.31823 | 0 | 0.248938 | 0.16128 | 0.17389 | 10 | 502.5447 | 0.789217 |
| 1346 | −0.36844 | 0 | 0.31303 | 0.214129 | 0.186465 | 0.210249 | 10 | 505.8741 | 0.786869 |
| 1347 | −0.37665 | −0.49016 | 0.415897 | 0.213769 | 0 | 0 | 10 | 497.2986 | 0.797567 |
| 1348 | −0.3692 | −0.49878 | 0.458935 | 0 | 0.257995 | 0 | 10 | 496.593 | 0.797934 |
| 1349 | −0.36789 | −0.4958 | 0.464972 | 0 | 0 | 0.255069 | 10 | 494.6915 | 0.799853 |
| 1350 | −0.36683 | −0.32775 | 0 | 0.247282 | 0.215748 | 0 | 10 | 500.5077 | 0.791728 |
| 1351 | −0.36769 | −0.32552 | 0 | 0.266766 | 0 | 0.223296 | 10 | 498.383 | 0.793912 |
| 1352 | −0.35346 | −0.32601 | 0 | 0 | 0.190682 | 0.171912 | 10 | 500.5661 | 0.792769 |
| 1353 | −0.36573 | 0 | 0.297911 | 0.217151 | 0.249518 | 0 | 10 | 505.9997 | 0.786155 |
| 1354 | −0.36751 | 0 | 0.304766 | 0.234822 | 0 | 0.26618 | 10 | 503.7464 | 0.788441 |
| 1355 | −0.35702 | 0 | 0.341819 | 0 | 0.21209 | 0.213821 | 10 | 504.9725 | 0.786461 |
| 1356 | −0.36116 | 0 | 0 | 0.256551 | 0.172681 | 0.188346 | 10 | 504.8422 | 0.786461 |
| 1357 | −0.3784 | −0.48038 | 0.429094 | 0.184036 | 0.235336 | 0 | 10 | 499.8157 | 0.793075 |
| 1358 | −0.37944 | −0.47686 | 0.432955 | 0.204998 | 0 | 0.247828 | 10 | 498.7842 | 0.794443 |
| 1359 | −0.37057 | −0.48826 | 0.469023 | 0 | 0.198485 | 0.194003 | 10 | 499.5116 | 0.792973 |
| 1360 | −0.36821 | −0.31823 | 0 | 0.248938 | 0.16128 | 0.17389 | 10 | 504.4733 | 0.786073 |
| 1361 | −0.36844 | 0 | 0.31303 | 0.214129 | 0.186465 | 0.210249 | 10 | 508.0266 | 0.781847 |
| 1362 | −0.3784 | −0.48038 | 0.429094 | 0.184036 | 0.235336 | 0 | 10 | 494.9577 | 0.798996 |
| 1363 | −0.37944 | −0.47686 | 0.432955 | 0.204998 | 0 | 0.247828 | 10 | 492.7013 | 0.801037 |
| 1364 | −0.37057 | −0.48826 | 0.469023 | 0 | 0.198485 | 0.194003 | 10 | 493.6389 | 0.800588 |
| 1365 | −0.36821 | −0.31823 | 0 | 0.248938 | 0.16128 | 0.17389 | 10 | 497.7825 | 0.795423 |
| 1366 | −0.36844 | 0 | 0.31303 | 0.214129 | 0.186465 | 0.210249 | 10 | 502.9278 | 0.789135 |
| 1367 | −0.38017 | −0.47149 | 0.43946 | 0.184707 | 0.175726 | 0.194494 | 10 | 498.192 | 0.795198 |
| 1368 | −0.34954 | 0 | 0.311734 | 0 | 0 | 0 | 10 | 547.7977 | 0.734403 |
| 1369 | −0.34838 | −0.35299 | 0 | 0 | 0 | 0 | 10 | 555.1728 | 0.719459 |
| 1370 | −0.35731 | 0 | 0 | 0.283735 | 0 | 0 | 10 | 553.422 | 0.72393 |
| 1371 | −0.34421 | 0 | 0 | 0 | 0.260301 | 0 | 10 | 553.4915 | 0.725972 |
| 1372 | −0.34349 | 0 | 0 | 0 | 0 | 0.25144 | 10 | 552.4178 | 0.727135 |
| 1373 | −0.34604 | 0 | 0 | 0 | 0.20186 | 0.189915 | 10 | 553.9356 | 0.723175 |
| 1374 | −0.36484 | −0.51223 | 0.448932 | 0 | 0 | 0 | 10 | 541.011 | 0.740344 |
| 1375 | −0.36535 | −0.34122 | 0 | 0.272545 | 0 | 0 | 10 | 553.6545 | 0.721154 |
| 1376 | −0.3635 | 0 | 0.280897 | 0.247949 | 0 | 0 | 10 | 546.5241 | 0.734587 |
| 1377 | −0.35247 | −0.33749 | 0 | 0 | 0.244058 | 0 | 10 | 553.8237 | 0.725012 |
| 1378 | −0.3509 | −0.33426 | 0 | 0 | 0 | 0.230316 | 10 | 554.1737 | 0.720746 |
| 1379 | −0.35435 | 0 | 0.326983 | 0 | 0.276248 | 0 | 10 | 544.2775 | 0.737384 |
| 1380 | −0.35429 | 0 | 0.335115 | 0 | 0 | 0.278056 | 10 | 544.012 | 0.737833 |
| 1381 | −0.35918 | 0 | 0 | 0.257296 | 0.231108 | 0 | 10 | 552.3845 | 0.724788 |
| 1382 | −0.36046 | 0 | 0 | 0.27483 | 0 | 0.241127 | 10 | 551.8039 | 0.724604 |
| 1383 | −0.34604 | 0 | 0 | 0 | 0.20186 | 0.189915 | 10 | 550.5639 | 0.728809 |
| 1384 | −0.36484 | −0.51223 | 0.448932 | 0 | 0 | 0 | 10 | 538.9378 | 0.742589 |
| 1385 | −0.36535 | −0.34122 | 0 | 0.272545 | 0 | 0 | 10 | 551.0392 | 0.725625 |
| 1386 | −0.3635 | 0 | 0.280897 | 0.247949 | 0 | 0 | 10 | 544.8759 | 0.736547 |
| 1387 | −0.35247 | −0.33749 | 0 | 0 | 0.244058 | 0 | 10 | 551.0016 | 0.725829 |
| 1388 | −0.3509 | −0.33426 | 0 | 0 | 0 | 0.230316 | 10 | 550.3076 | 0.726503 |
| 1389 | −0.35435 | 0 | 0.326983 | 0 | 0.276248 | 0 | 10 | 542.345 | 0.74018 |
| 1390 | −0.35429 | 0 | 0.335115 | 0 | 0 | 0.278056 | 10 | 540.8185 | 0.743365 |
| 1391 | −0.35918 | 0 | 0 | 0.257296 | 0.231108 | 0 | 10 | 549.9284 | 0.728728 |
| 1392 | −0.36046 | 0 | 0 | 0.27483 | 0 | 0.241127 | 10 | 548.1903 | 0.732504 |
| 1393 | −0.37665 | −0.49016 | 0.415897 | 0.213769 | 0 | 0 | 10 | 538.9569 | 0.74163 |
| 1394 | −0.3692 | −0.49878 | 0.458935 | 0 | 0.257995 | 0 | 10 | 536.1014 | 0.746693 |
| 1395 | −0.36789 | −0.4958 | 0.464972 | 0 | 0 | 0.255069 | 10 | 536.3744 | 0.745937 |
| 1396 | −0.36683 | −0.32775 | 0 | 0.247282 | 0.215748 | 0 | 10 | 550.69 | 0.727074 |
| 1397 | −0.36769 | −0.32552 | 0 | 0.266766 | 0 | 0.223296 | 10 | 550.3766 | 0.724624 |
| 1398 | −0.35346 | −0.32601 | 0 | 0 | 0.190682 | 0.171912 | 10 | 552.3108 | 0.724175 |
| 1399 | −0.36573 | 0 | 0.297911 | 0.217151 | 0.249518 | 0 | 10 | 542.3188 | 0.740078 |
| 1400 | −0.36751 | 0 | 0.304766 | 0.234822 | 0 | 0.26618 | 10 | 541.5699 | 0.740711 |
| 1401 | −0.35702 | 0 | 0.341819 | 0 | 0.21209 | 0.213821 | 10 | 541.4767 | 0.739343 |
| 1402 | −0.36116 | 0 | 0 | 0.256551 | 0.172681 | 0.188346 | 10 | 550.5607 | 0.726625 |
| 1403 | −0.34604 | 0 | 0 | 0 | 0.20186 | 0.189915 | 10 | 550.7113 | 0.729422 |
| 1404 | −0.36484 | −0.51223 | 0.448932 | 0 | 0 | 0 | 10 | 539.1563 | 0.742038 |
| 1405 | −0.36535 | −0.34122 | 0 | 0.272545 | 0 | 0 | 10 | 550.8417 | 0.725298 |
| 1406 | −0.35247 | −0.33749 | 0 | 0 | 0.244058 | 0 | 10 | 551.2899 | 0.726543 |
| 1407 | −0.3635 | 0 | 0.280897 | 0.247949 | 0 | 0 | 10 | 544.9086 | 0.736832 |
| 1408 | −0.3509 | −0.33426 | 0 | 0 | 0 | 0.230316 | 10 | 550.0403 | 0.727482 |
| 1409 | −0.35435 | 0 | 0.326983 | 0 | 0.276248 | 0 | 10 | 543.1955 | 0.737608 |
| 1410 | −0.35429 | 0 | 0.335115 | 0 | 0 | 0.278056 | 10 | 540.8379 | 0.743692 |
| 1411 | −0.35918 | 0 | 0 | 0.257296 | 0.231108 | 0 | 10 | 550.2132 | 0.729463 |
| 1412 | −0.36046 | 0 | 0 | 0.27483 | 0 | 0.241127 | 10 | 547.8994 | 0.733199 |
| 1413 | −0.37665 | −0.49016 | 0.415897 | 0.213769 | 0 | 0 | 10 | 539.6268 | 0.741997 |
| 1414 | −0.3692 | −0.49878 | 0.458935 | 0 | 0.257995 | 0 | 10 | 537.7514 | 0.743467 |
| 1415 | −0.36789 | −0.4958 | 0.464972 | 0 | 0 | 0.255069 | 10 | 537.1671 | 0.744733 |
| 1416 | −0.36683 | −0.32775 | 0 | 0.247282 | 0.215748 | 0 | 10 | 551.494 | 0.724441 |
| 1417 | −0.36769 | −0.32552 | 0 | 0.266766 | 0 | 0.223296 | 10 | 550.6401 | 0.72393 |
| 1418 | −0.35346 | −0.32601 | 0 | 0 | 0.190682 | 0.171912 | 10 | 553.0071 | 0.723093 |
| 1419 | −0.36573 | 0 | 0.297911 | 0.217151 | 0.249518 | 0 | 10 | 543.73 | 0.736628 |
| 1420 | −0.36751 | 0 | 0.304766 | 0.234822 | 0 | 0.26618 | 10 | 542.2033 | 0.738629 |
| 1421 | −0.35702 | 0 | 0.341819 | 0 | 0.21209 | 0.213821 | 10 | 542.8711 | 0.737343 |
| 1422 | −0.36116 | 0 | 0 | 0.256551 | 0.172681 | 0.188346 | 10 | 551.3179 | 0.724379 |
| 1423 | −0.37665 | −0.49016 | 0.415897 | 0.213769 | 0 | 0 | 10 | 537.0063 | 0.745917 |
| 1424 | −0.3692 | −0.49878 | 0.458935 | 0 | 0.257995 | 0 | 10 | 534.6157 | 0.747652 |
| 1425 | −0.36789 | −0.4958 | 0.464972 | 0 | 0 | 0.255069 | 10 | 532.8451 | 0.751511 |
| 1426 | −0.36683 | −0.32775 | 0 | 0.247282 | 0.215748 | 0 | 10 | 548.007 | 0.730055 |
| 1427 | −0.36769 | −0.32552 | 0 | 0.266766 | 0 | 0.223296 | 10 | 546.0434 | 0.732892 |
| 1428 | −0.35346 | −0.32601 | 0 | 0 | 0.190682 | 0.171912 | 10 | 548.6151 | 0.728177 |
| 1429 | −0.36573 | 0 | 0.297911 | 0.217151 | 0.249518 | 0 | 10 | 541.0693 | 0.740323 |
| 1430 | −0.36751 | 0 | 0.304766 | 0.234822 | 0 | 0.26618 | 10 | 538.2775 | 0.745754 |
| 1431 | −0.35702 | 0 | 0.341819 | 0 | 0.21209 | 0.213821 | 10 | 538.9845 | 0.742406 |
| 1432 | −0.36116 | 0 | 0 | 0.256551 | 0.172681 | 0.188346 | 10 | 547.1116 | 0.731953 |
| 1433 | −0.3784 | −0.48038 | 0.429094 | 0.184036 | 0.235336 | 0 | 10 | 536.4386 | 0.745652 |
| 1434 | −0.37944 | −0.47686 | 0.432955 | 0.204998 | 0 | 0.247828 | 10 | 535.412 | 0.746897 |
| 1435 | −0.37057 | −0.48826 | 0.469023 | 0 | 0.198485 | 0.194003 | 10 | 535.3866 | 0.745999 |
| 1436 | −0.36821 | −0.31823 | 0 | 0.248938 | 0.16128 | 0.17389 | 10 | 549.9253 | 0.725543 |
| 1437 | −0.36844 | 0 | 0.31303 | 0.214129 | 0.186465 | 0.210249 | 10 | 541.0002 | 0.73916 |
| 1438 | −0.34604 | 0 | 0 | 0 | 0.20186 | 0.189915 | 10 | 549.9698 | 0.731137 |
| 1439 | −0.36484 | −0.51223 | 0.448932 | 0 | 0 | 0 | 10 | 538.9439 | 0.743896 |
| 1440 | −0.36535 | −0.34122 | 0 | 0.272545 | 0 | 0 | 10 | 550.6129 | 0.727829 |
| 1441 | −0.35247 | −0.33749 | 0 | 0 | 0.244058 | 0 | 10 | 550.6872 | 0.728891 |
| 1442 | −0.3635 | 0 | 0.280897 | 0.247949 | 0 | 0 | 10 | 544.4644 | 0.737118 |
| 1443 | −0.3509 | −0.33426 | 0 | 0 | 0 | 0.230316 | 10 | 549.8402 | 0.729156 |
| 1444 | −0.35435 | 0 | 0.326983 | 0 | 0.276248 | 0 | 10 | 542.0769 | 0.740119 |
| 1445 | −0.35429 | 0 | 0.335115 | 0 | 0 | 0.278056 | 10 | 540.3579 | 0.744855 |
| 1446 | −0.35918 | 0 | 0 | 0.257296 | 0.231108 | 0 | 10 | 549.3649 | 0.730361 |
| 1447 | −0.36046 | 0 | 0 | 0.27483 | 0 | 0.241127 | 10 | 547.4602 | 0.734587 |
| 1448 | −0.37665 | −0.49016 | 0.415897 | 0.213769 | 0 | 0 | 10 | 538.6897 | 0.742242 |
| 1449 | −0.3692 | −0.49878 | 0.458935 | 0 | 0.257995 | 0 | 10 | 535.9203 | 0.747366 |
| 1450 | −0.36789 | −0.4958 | 0.464972 | 0 | 0 | 0.255069 | 10 | 536.0729 | 0.746713 |
| 1451 | −0.36683 | −0.32775 | 0 | 0.247282 | 0.215748 | 0 | 10 | 550.1306 | 0.727952 |
| 1452 | −0.36769 | −0.32552 | 0 | 0.266766 | 0 | 0.223296 | 10 | 549.7396 | 0.726686 |
| 1453 | −0.35346 | −0.32601 | 0 | 0 | 0.190682 | 0.171912 | 10 | 551.7239 | 0.726952 |
| 1454 | −0.36573 | 0 | 0.297911 | 0.217151 | 0.249518 | 0 | 10 | 541.9103 | 0.740895 |
| 1455 | −0.36751 | 0 | 0.304766 | 0.234822 | 0 | 0.26618 | 10 | 541.0657 | 0.742508 |
| 1456 | −0.35702 | 0 | 0.341819 | 0 | 0.21209 | 0.213821 | 10 | 541.0526 | 0.741671 |
| 1457 | −0.36116 | 0 | 0 | 0.256551 | 0.172681 | 0.188346 | 10 | 549.8914 | 0.727523 |
| 1458 | −0.37665 | −0.49016 | 0.415897 | 0.213769 | 0 | 0 | 10 | 536.552 | 0.746325 |
| 1459 | −0.3692 | −0.49878 | 0.458935 | 0 | 0.257995 | 0 | 10 | 533.5107 | 0.751123 |
| 1460 | −0.36789 | −0.4958 | 0.464972 | 0 | 0 | 0.255069 | 10 | 532.3705 | 0.753042 |
| 1461 | −0.36683 | −0.32775 | 0 | 0.247282 | 0.215748 | 0 | 10 | 547.2016 | 0.732362 |
| 1462 | −0.36769 | −0.32552 | 0 | 0.266766 | 0 | 0.223296 | 10 | 545.6103 | 0.734015 |
| 1463 | −0.35346 | −0.32601 | 0 | 0 | 0.190682 | 0.171912 | 10 | 547.9305 | 0.730443 |
| 1464 | −0.36573 | 0 | 0.297911 | 0.217151 | 0.249518 | 0 | 10 | 539.8497 | 0.743937 |
| 1465 | −0.36751 | 0 | 0.304766 | 0.234822 | 0 | 0.26618 | 10 | 537.6497 | 0.74804 |
| 1466 | −0.35702 | 0 | 0.341819 | 0 | 0.21209 | 0.213821 | 10 | 537.8504 | 0.746734 |
| 1467 | −0.36116 | 0 | 0 | 0.256551 | 0.172681 | 0.188346 | 10 | 546.2339 | 0.734423 |
| 1468 | −0.3784 | −0.48038 | 0.429094 | 0.184036 | 0.235336 | 0 | 10 | 534.4945 | 0.749592 |
| 1469 | −0.37944 | −0.47686 | 0.432955 | 0.204998 | 0 | 0.247828 | 10 | 534.1497 | 0.749633 |
| 1470 | −0.37057 | −0.48826 | 0.469023 | 0 | 0.198485 | 0.194003 | 10 | 533.4755 | 0.749755 |
| 1471 | −0.36821 | −0.31823 | 0 | 0.248938 | 0.16128 | 0.17389 | 10 | 548.5354 | 0.728503 |
| 1472 | −0.36844 | 0 | 0.31303 | 0.214129 | 0.186465 | 0.210249 | 10 | 539.1744 | 0.743916 |
| 1473 | −0.37665 | −0.49016 | 0.415897 | 0.213769 | 0 | 0 | 10 | 536.7824 | 0.746529 |
| 1474 | −0.3692 | −0.49878 | 0.458935 | 0 | 0.257995 | 0 | 10 | 534.4505 | 0.748591 |
| 1475 | −0.36789 | −0.4958 | 0.464972 | 0 | 0 | 0.255069 | 10 | 532.6001 | 0.752225 |
| 1476 | −0.36683 | −0.32775 | 0 | 0.247282 | 0.215748 | 0 | 10 | 547.486 | 0.731627 |
| 1477 | −0.36769 | −0.32552 | 0 | 0.266766 | 0 | 0.223296 | 10 | 545.4243 | 0.735036 |
| 1478 | −0.35346 | −0.32601 | 0 | 0 | 0.190682 | 0.171912 | 10 | 548.0851 | 0.730708 |
| 1479 | −0.36573 | 0 | 0.297911 | 0.217151 | 0.249518 | 0 | 10 | 540.633 | 0.740895 |
| 1480 | −0.36751 | 0 | 0.304766 | 0.234822 | 0 | 0.26618 | 10 | 537.7185 | 0.747101 |
| 1481 | −0.35702 | 0 | 0.341819 | 0 | 0.21209 | 0.213821 | 10 | 538.5293 | 0.744713 |
| 1482 | −0.36116 | 0 | 0 | 0.256551 | 0.172681 | 0.188346 | 10 | 546.3988 | 0.734607 |
| 1483 | −0.3784 | −0.48038 | 0.429094 | 0.184036 | 0.235336 | 0 | 10 | 536.221 | 0.746468 |
| 1484 | −0.37944 | −0.47686 | 0.432955 | 0.204998 | 0 | 0.247828 | 10 | 535.1608 | 0.748224 |
| 1485 | −0.37057 | −0.48826 | 0.469023 | 0 | 0.198485 | 0.194003 | 10 | 535.1965 | 0.747652 |
| 1486 | −0.36821 | −0.31823 | 0 | 0.248938 | 0.16128 | 0.17389 | 10 | 549.4426 | 0.725727 |
| 1487 | −0.36844 | 0 | 0.31303 | 0.214129 | 0.186465 | 0.210249 | 10 | 540.7635 | 0.741385 |
| 1488 | −0.3784 | −0.48038 | 0.429094 | 0.184036 | 0.235336 | 0 | 10 | 532.8984 | 0.750327 |
| 1489 | −0.37944 | −0.47686 | 0.432955 | 0.204998 | 0 | 0.247828 | 10 | 530.5292 | 0.754348 |
| 1490 | −0.37057 | −0.48826 | 0.469023 | 0 | 0.198485 | 0.194003 | 10 | 530.6014 | 0.75345 |
| 1491 | −0.36821 | −0.31823 | 0 | 0.248938 | 0.16128 | 0.17389 | 10 | 544.5851 | 0.733913 |
| 1492 | −0.36844 | 0 | 0.31303 | 0.214129 | 0.186465 | 0.210249 | 10 | 536.4256 | 0.746346 |
| 1493 | −0.38017 | −0.47149 | 0.43946 | 0.184707 | 0.175726 | 0.194494 | 10 | 533.9562 | 0.747428 |
| 1494 | −0.34604 | 0 | 0 | 0 | 0.20186 | 0.189915 | 10 | 550.7942 | 0.728932 |
| 1495 | −0.36484 | −0.51223 | 0.448932 | 0 | 0 | 0 | 10 | 540.0674 | 0.740609 |
| 1496 | −0.36535 | −0.34122 | 0 | 0.272545 | 0 | 0 | 10 | 551.1287 | 0.726094 |
| 1497 | −0.35247 | −0.33749 | 0 | 0 | 0.244058 | 0 | 10 | 551.3507 | 0.727176 |
| 1498 | −0.3635 | 0 | 0.280897 | 0.247949 | 0 | 0 | 10 | 545.501 | 0.736404 |
| 1499 | −0.3509 | −0.33426 | 0 | 0 | 0 | 0.230316 | 10 | 550.4231 | 0.726584 |
| 1500 | −0.35435 | 0 | 0.326983 | 0 | 0.276248 | 0 | 10 | 543.4894 | 0.736363 |
| 1501 | −0.35429 | 0 | 0.335115 | 0 | 0 | 0.278056 | 10 | 541.6245 | 0.742896 |
| 1502 | −0.35918 | 0 | 0 | 0.257296 | 0.231108 | 0 | 10 | 550.159 | 0.729136 |
| 1503 | −0.36046 | 0 | 0 | 0.27483 | 0 | 0.241127 | 10 | 548.1668 | 0.73177 |
| 1504 | −0.37665 | −0.49016 | 0.415897 | 0.213769 | 0 | 0 | 10 | 539.761 | 0.741283 |
| 1505 | −0.3692 | −0.49878 | 0.458935 | 0 | 0.257995 | 0 | 10 | 537.3557 | 0.74506 |
| 1506 | −0.36789 | −0.4958 | 0.464972 | 0 | 0 | 0.255069 | 10 | 537.2476 | 0.744753 |
| 1507 | −0.36683 | −0.32775 | 0 | 0.247282 | 0.215748 | 0 | 10 | 550.764 | 0.728279 |
| 1508 | −0.36769 | −0.32552 | 0 | 0.266766 | 0 | 0.223296 | 10 | 550.2089 | 0.725951 |
| 1509 | −0.35346 | −0.32601 | 0 | 0 | 0.190682 | 0.171912 | 10 | 552.3553 | 0.724808 |
| 1510 | −0.36573 | 0 | 0.297911 | 0.217151 | 0.249518 | 0 | 10 | 543.1225 | 0.738261 |
| 1511 | −0.36751 | 0 | 0.304766 | 0.234822 | 0 | 0.26618 | 10 | 541.9958 | 0.740691 |
| 1512 | −0.35702 | 0 | 0.341819 | 0 | 0.21209 | 0.213821 | 10 | 542.32 | 0.73918 |
| 1513 | −0.36116 | 0 | 0 | 0.256551 | 0.172681 | 0.188346 | 10 | 550.4946 | 0.726952 |
| 1514 | −0.37665 | −0.49016 | 0.415897 | 0.213769 | 0 | 0 | 10 | 537.569 | 0.745223 |
| 1515 | −0.3692 | −0.49878 | 0.458935 | 0 | 0.257995 | 0 | 10 | 534.9298 | 0.747326 |
| 1516 | −0.36789 | −0.4958 | 0.464972 | 0 | 0 | 0.255069 | 10 | 533.6018 | 0.750796 |
| 1517 | −0.36683 | −0.32775 | 0 | 0.247282 | 0.215748 | 0 | 10 | 547.7879 | 0.731075 |
| 1518 | −0.36769 | −0.32552 | 0 | 0.266766 | 0 | 0.223296 | 10 | 546.1147 | 0.733444 |
| 1519 | −0.35346 | −0.32601 | 0 | 0 | 0.190682 | 0.171912 | 10 | 548.5452 | 0.728952 |
| 1520 | −0.36573 | 0 | 0.297911 | 0.217151 | 0.249518 | 0 | 10 | 541.0443 | 0.740385 |
| 1521 | −0.36751 | 0 | 0.304766 | 0.234822 | 0 | 0.26618 | 10 | 538.6687 | 0.746223 |
| 1522 | −0.35702 | 0 | 0.341819 | 0 | 0.21209 | 0.213821 | 10 | 539.2029 | 0.744427 |
| 1523 | −0.36116 | 0 | 0 | 0.256551 | 0.172681 | 0.188346 | 10 | 546.9042 | 0.733117 |
| 1524 | −0.3784 | −0.48038 | 0.429094 | 0.184036 | 0.235336 | 0 | 10 | 535.7025 | 0.747836 |
| 1525 | −0.37944 | −0.47686 | 0.432955 | 0.204998 | 0 | 0.247828 | 10 | 535.0671 | 0.74902 |
| 1526 | −0.37057 | −0.48826 | 0.469023 | 0 | 0.198485 | 0.194003 | 10 | 534.7329 | 0.747244 |
| 1527 | −0.36821 | −0.31823 | 0 | 0.248938 | 0.16128 | 0.17389 | 10 | 548.9309 | 0.728054 |
| 1528 | −0.36844 | 0 | 0.31303 | 0.214129 | 0.186465 | 0.210249 | 10 | 540.0787 | 0.742651 |
| 1529 | −0.37665 | −0.49016 | 0.415897 | 0.213769 | 0 | 0 | 10 | 538.1232 | 0.744039 |
| 1530 | −0.3692 | −0.49878 | 0.458935 | 0 | 0.257995 | 0 | 10 | 536.3067 | 0.744733 |
| 1531 | −0.36789 | −0.4958 | 0.464972 | 0 | 0 | 0.255069 | 10 | 534.1803 | 0.749224 |
| 1532 | −0.36683 | −0.32775 | 0 | 0.247282 | 0.215748 | 0 | 10 | 548.4098 | 0.729075 |
| 1533 | −0.36769 | −0.32552 | 0 | 0.266766 | 0 | 0.223296 | 10 | 546.2029 | 0.733158 |
| 1534 | −0.35346 | −0.32601 | 0 | 0 | 0.190682 | 0.171912 | 10 | 549.0467 | 0.729116 |
| 1535 | −0.36573 | 0 | 0.297911 | 0.217151 | 0.249518 | 0 | 10 | 542.2205 | 0.737812 |
| 1536 | −0.36751 | 0 | 0.304766 | 0.234822 | 0 | 0.26618 | 10 | 539.078 | 0.743855 |
| 1537 | −0.35702 | 0 | 0.341819 | 0 | 0.21209 | 0.213821 | 10 | 540.2858 | 0.741058 |
| 1538 | −0.36116 | 0 | 0 | 0.256551 | 0.172681 | 0.188346 | 10 | 547.4074 | 0.73128 |
| 1539 | −0.3784 | −0.48038 | 0.429094 | 0.184036 | 0.235336 | 0 | 10 | 537.705 | 0.744855 |
| 1540 | −0.37944 | −0.47686 | 0.432955 | 0.204998 | 0 | 0.247828 | 10 | 536.2983 | 0.746958 |
| 1541 | −0.37057 | −0.48826 | 0.469023 | 0 | 0.198485 | 0.194003 | 10 | 536.7048 | 0.745305 |
| 1542 | −0.36821 | −0.31823 | 0 | 0.248938 | 0.16128 | 0.17389 | 10 | 550.0061 | 0.725604 |
| 1543 | −0.36844 | 0 | 0.31303 | 0.214129 | 0.186465 | 0.210249 | 10 | 541.8439 | 0.738731 |
| 1544 | −0.3784 | −0.48038 | 0.429094 | 0.184036 | 0.235336 | 0 | 10 | 534.5149 | 0.748755 |
| 1545 | −0.37944 | −0.47686 | 0.432955 | 0.204998 | 0 | 0.247828 | 10 | 531.8856 | 0.753307 |
| 1546 | −0.37057 | −0.48826 | 0.469023 | 0 | 0.198485 | 0.194003 | 10 | 532.3781 | 0.74953 |
| 1547 | −0.36821 | −0.31823 | 0 | 0.248938 | 0.16128 | 0.17389 | 10 | 545.3535 | 0.732076 |
| 1548 | −0.36844 | 0 | 0.31303 | 0.214129 | 0.186465 | 0.210249 | 10 | 537.8118 | 0.744713 |
| 1549 | −0.38017 | −0.47149 | 0.43946 | 0.184707 | 0.175726 | 0.194494 | 10 | 535.0461 | 0.747734 |
| 1550 | −0.37665 | −0.49016 | 0.415897 | 0.213769 | 0 | 0 | 10 | 537.6997 | 0.745488 |
| 1551 | −0.3692 | −0.49878 | 0.458935 | 0 | 0.257995 | 0 | 10 | 535.1854 | 0.747775 |
| 1552 | −0.36789 | −0.4958 | 0.464972 | 0 | 0 | 0.255069 | 10 | 533.7112 | 0.750959 |
| 1553 | −0.36683 | −0.32775 | 0 | 0.247282 | 0.215748 | 0 | 10 | 547.6116 | 0.731198 |
| 1554 | −0.36769 | −0.32552 | 0 | 0.266766 | 0 | 0.223296 | 10 | 545.7751 | 0.735036 |
| 1555 | −0.35346 | −0.32601 | 0 | 0 | 0.190682 | 0.171912 | 10 | 548.3733 | 0.730443 |
| 1556 | −0.36573 | 0 | 0.297911 | 0.217151 | 0.249518 | 0 | 10 | 540.934 | 0.740078 |
| 1557 | −0.36751 | 0 | 0.304766 | 0.234822 | 0 | 0.26618 | 10 | 538.3822 | 0.745692 |
| 1558 | −0.35702 | 0 | 0.341819 | 0 | 0.21209 | 0.213821 | 10 | 539.0757 | 0.744917 |
| 1559 | −0.36116 | 0 | 0 | 0.256551 | 0.172681 | 0.188346 | 10 | 546.4664 | 0.734607 |
| 1560 | −0.3784 | −0.48038 | 0.429094 | 0.184036 | 0.235336 | 0 | 10 | 535.6325 | 0.748816 |
| 1561 | −0.37944 | −0.47686 | 0.432955 | 0.204998 | 0 | 0.247828 | 10 | 534.886 | 0.749224 |
| 1562 | −0.37057 | −0.48826 | 0.469023 | 0 | 0.198485 | 0.194003 | 10 | 534.628 | 0.748101 |
| 1563 | −0.36821 | −0.31823 | 0 | 0.248938 | 0.16128 | 0.17389 | 10 | 548.4697 | 0.72983 |
| 1564 | −0.36844 | 0 | 0.31303 | 0.214129 | 0.186465 | 0.210249 | 10 | 539.7958 | 0.742406 |
| 1565 | −0.3784 | −0.48038 | 0.429094 | 0.184036 | 0.235336 | 0 | 10 | 533.2466 | 0.752429 |
| 1566 | −0.37944 | −0.47686 | 0.432955 | 0.204998 | 0 | 0.247828 | 10 | 531.1839 | 0.755594 |
| 1567 | −0.37057 | −0.48826 | 0.469023 | 0 | 0.198485 | 0.194003 | 10 | 531.207 | 0.75396 |
| 1568 | −0.36821 | −0.31823 | 0 | 0.248938 | 0.16128 | 0.17389 | 10 | 544.4734 | 0.735056 |
| 1569 | −0.36844 | 0 | 0.31303 | 0.214129 | 0.186465 | 0.210249 | 10 | 536.5127 | 0.747816 |
| 1570 | −0.38017 | −0.47149 | 0.43946 | 0.184707 | 0.175726 | 0.194494 | 10 | 532.8809 | 0.752144 |
| 1571 | −0.3784 | −0.48038 | 0.429094 | 0.184036 | 0.235336 | 0 | 10 | 534.4833 | 0.749102 |
| 1572 | −0.37944 | −0.47686 | 0.432955 | 0.204998 | 0 | 0.247828 | 10 | 531.7734 | 0.753083 |
| 1573 | −0.37057 | −0.48826 | 0.469023 | 0 | 0.198485 | 0.194003 | 10 | 532.3423 | 0.74951 |
| 1574 | −0.36821 | −0.31823 | 0 | 0.248938 | 0.16128 | 0.17389 | 10 | 544.972 | 0.734403 |
| 1575 | −0.36844 | 0 | 0.31303 | 0.214129 | 0.186465 | 0.210249 | 10 | 537.5338 | 0.745019 |
| 1576 | −0.38017 | −0.47149 | 0.43946 | 0.184707 | 0.175726 | 0.194494 | 10 | 534.9657 | 0.747366 |
| 1577 | −0.38017 | −0.47149 | 0.43946 | 0.184707 | 0.175726 | 0.194494 | 10 | 530.3792 | 0.754287 |
| 1578 | −0.3784 | −0.48038 | 0.429094 | 0.184036 | 0.235336 | 0 | 10 | 557.5194 | 0.713907 |
| 1579 | −0.37944 | −0.47686 | 0.432955 | 0.204998 | 0 | 0.247828 | 10 | 555.7853 | 0.715968 |
| 1580 | −0.37057 | −0.48826 | 0.469023 | 0 | 0.198485 | 0.194003 | 10 | 555.6726 | 0.714029 |
| 1581 | −0.36821 | −0.31823 | 0 | 0.248938 | 0.16128 | 0.17389 | 10 | 566.9668 | 0.698575 |
| 1582 | −0.36844 | 0 | 0.31303 | 0.214129 | 0.186465 | 0.210249 | 10 | 566.256 | 0.696779 |
| 1583 | −0.38017 | −0.47149 | 0.43946 | 0.184707 | 0.175726 | 0.194494 | 10 | 557.0165 | 0.71064 |
| 1584 | −0.38017 | −0.47149 | 0.43946 | 0.184707 | 0.175726 | 0.194494 | 10 | 552.7672 | 0.718071 |
| 1585 | −0.38017 | −0.47149 | 0.43946 | 0.184707 | 0.175726 | 0.194494 | 10 | 553.4193 | 0.716213 |
| 1586 | −0.34954 | 0 | 0.311734 | 0 | 0 | 0 | 9 | 551.2231 | 0.722154 |
| 1587 | −0.34838 | −0.35299 | 0 | 0 | 0 | 0 | 9 | 539.803 | 0.743631 |
| 1588 | −0.35731 | 0 | 0 | 0.283735 | 0 | 0 | 9 | 548.6675 | 0.730422 |
| 1589 | −0.34421 | 0 | 0 | 0 | 0.260301 | 0 | 9 | 546.8469 | 0.730994 |
| 1590 | −0.34349 | 0 | 0 | 0 | 0 | 0.25144 | 9 | 548.4876 | 0.73128 |
| 1591 | −0.34954 | 0 | 0.311734 | 0 | 0 | 0 | 9 | 548.6006 | 0.72734 |
| 1592 | −0.34838 | −0.35299 | 0 | 0 | 0 | 0 | 9 | 535.4831 | 0.750959 |
| 1593 | −0.35731 | 0 | 0 | 0.283735 | 0 | 0 | 9 | 545.2817 | 0.736302 |
| 1594 | −0.34421 | 0 | 0 | 0 | 0.260301 | 0 | 9 | 543.1246 | 0.73771 |
| 1595 | −0.34349 | 0 | 0 | 0 | 0 | 0.25144 | 9 | 543.1959 | 0.739119 |
| 1596 | −0.34604 | 0 | 0 | 0 | 0.20186 | 0.189915 | 9 | 545.7203 | 0.734505 |
| 1597 | −0.36484 | −0.51223 | 0.448932 | 0 | 0 | 0 | 9 | 532.5224 | 0.751613 |
| 1598 | −0.3635 | 0 | 0.280897 | 0.247949 | 0 | 0 | 9 | 548.4163 | 0.729136 |
| 1599 | −0.36535 | −0.34122 | 0 | 0.272545 | 0 | 0 | 9 | 536.4605 | 0.751021 |
| 1600 | −0.35247 | −0.33749 | 0 | 0 | 0.244058 | 0 | 9 | 535.0354 | 0.751 |
| 1601 | −0.3509 | −0.33426 | 0 | 0 | 0 | 0.230316 | 9 | 537.0625 | 0.747285 |
| 1602 | −0.35435 | 0 | 0.326983 | 0 | 0.276248 | 0 | 9 | 545.1178 | 0.734097 |
| 1603 | −0.35429 | 0 | 0.335115 | 0 | 0 | 0.278056 | 9 | 547.0084 | 0.730545 |
| 1604 | −0.35918 | 0 | 0 | 0.257296 | 0.231108 | 0 | 9 | 544.2046 | 0.736894 |
| 1605 | −0.36046 | 0 | 0 | 0.27483 | 0 | 0.241127 | 9 | 545.1932 | 0.737976 |
| 1606 | −0.34954 | 0 | 0.311734 | 0 | 0 | 0 | 9 | 547.6854 | 0.728299 |
| 1607 | −0.34838 | −0.35299 | 0 | 0 | 0 | 0 | 9 | 534.6682 | 0.751062 |
| 1608 | −0.35731 | 0 | 0 | 0.283735 | 0 | 0 | 9 | 544.4374 | 0.736649 |
| 1609 | −0.34421 | 0 | 0 | 0 | 0.260301 | 0 | 9 | 542.5898 | 0.737139 |
| 1610 | −0.34349 | 0 | 0 | 0 | 0 | 0.25144 | 9 | 542.1286 | 0.739568 |
| 1611 | −0.34604 | 0 | 0 | 0 | 0.20186 | 0.189915 | 9 | 545.7162 | 0.731953 |
| 1612 | −0.36484 | −0.51223 | 0.448932 | 0 | 0 | 0 | 9 | 532.487 | 0.75196 |
| 1613 | −0.3635 | 0 | 0.280897 | 0.247949 | 0 | 0 | 9 | 548.0715 | 0.728524 |
| 1614 | −0.36535 | −0.34122 | 0 | 0.272545 | 0 | 0 | 9 | 536.2356 | 0.750694 |
| 1615 | −0.35247 | −0.33749 | 0 | 0 | 0.244058 | 0 | 9 | 535.4373 | 0.748122 |
| 1616 | −0.3509 | −0.33426 | 0 | 0 | 0 | 0.230316 | 9 | 536.7081 | 0.748081 |
| 1617 | −0.35435 | 0 | 0.326983 | 0 | 0.276248 | 0 | 9 | 545.469 | 0.731239 |
| 1618 | −0.35429 | 0 | 0.335115 | 0 | 0 | 0.278056 | 9 | 546.6231 | 0.731382 |
| 1619 | −0.35918 | 0 | 0 | 0.257296 | 0.231108 | 0 | 9 | 544.4594 | 0.732994 |
| 1620 | −0.36046 | 0 | 0 | 0.27483 | 0 | 0.241127 | 9 | 544.849 | 0.736077 |
| 1621 | −0.34604 | 0 | 0 | 0 | 0.20186 | 0.189915 | 9 | 539.7079 | 0.742651 |
| 1622 | −0.36484 | −0.51223 | 0.448932 | 0 | 0 | 0 | 9 | 528.2821 | 0.758329 |
| 1623 | −0.3635 | 0 | 0.280897 | 0.247949 | 0 | 0 | 9 | 544.8436 | 0.734444 |
| 1624 | −0.36535 | −0.34122 | 0 | 0.272545 | 0 | 0 | 9 | 531.1224 | 0.758411 |
| 1625 | −0.35247 | −0.33749 | 0 | 0 | 0.244058 | 0 | 9 | 529.7649 | 0.7559 |
| 1626 | −0.3509 | −0.33426 | 0 | 0 | 0 | 0.230316 | 9 | 529.3685 | 0.75884 |
| 1627 | −0.35435 | 0 | 0.326983 | 0 | 0.276248 | 0 | 9 | 541.7511 | 0.738588 |
| 1628 | −0.35429 | 0 | 0.335115 | 0 | 0 | 0.278056 | 9 | 541.3488 | 0.739282 |
| 1629 | −0.35918 | 0 | 0 | 0.257296 | 0.231108 | 0 | 9 | 539.891 | 0.743365 |
| 1630 | −0.36046 | 0 | 0 | 0.27483 | 0 | 0.241127 | 9 | 538.6846 | 0.746713 |
| 1631 | −0.37665 | −0.49016 | 0.415897 | 0.213769 | 0 | 0 | 9 | 530.5183 | 0.756451 |
| 1632 | −0.3692 | −0.49878 | 0.458935 | 0 | 0.257995 | 0 | 9 | 527.7008 | 0.758452 |
| 1633 | −0.36789 | −0.4958 | 0.464972 | 0 | 0 | 0.255069 | 9 | 529.1658 | 0.754695 |
| 1634 | −0.36683 | −0.32775 | 0 | 0.247282 | 0.215748 | 0 | 9 | 533.0759 | 0.753756 |
| 1635 | −0.36769 | −0.32552 | 0 | 0.266766 | 0 | 0.223296 | 9 | 533.7073 | 0.754797 |
| 1636 | −0.35346 | −0.32601 | 0 | 0 | 0.190682 | 0.171912 | 9 | 534.7541 | 0.749918 |
| 1637 | −0.36573 | 0 | 0.297911 | 0.217151 | 0.249518 | 0 | 9 | 543.6924 | 0.735322 |
| 1638 | −0.36751 | 0 | 0.304766 | 0.234822 | 0 | 0.26618 | 9 | 544.2523 | 0.736077 |
| 1639 | −0.35702 | 0 | 0.341819 | 0 | 0.21209 | 0.213821 | 9 | 543.9333 | 0.735138 |
| 1640 | −0.36116 | 0 | 0 | 0.256551 | 0.172681 | 0.188346 | 9 | 543.2477 | 0.73669 |
| 1641 | −0.34954 | 0 | 0.311734 | 0 | 0 | 0 | 9 | 548.6726 | 0.726441 |
| 1642 | −0.34838 | −0.35299 | 0 | 0 | 0 | 0 | 9 | 535.7106 | 0.749204 |
| 1643 | −0.35731 | 0 | 0 | 0.283735 | 0 | 0 | 9 | 545.1093 | 0.735587 |
| 1644 | −0.34421 | 0 | 0 | 0 | 0.260301 | 0 | 9 | 543.1302 | 0.737567 |
| 1645 | −0.34349 | 0 | 0 | 0 | 0 | 0.25144 | 9 | 542.8988 | 0.738364 |
| 1646 | −0.34604 | 0 | 0 | 0 | 0.20186 | 0.189915 | 9 | 544.8658 | 0.734832 |
| 1647 | −0.36484 | −0.51223 | 0.448932 | 0 | 0 | 0 | 9 | 532.5094 | 0.751613 |
| 1648 | −0.3635 | 0 | 0.280897 | 0.247949 | 0 | 0 | 9 | 547.8986 | 0.728891 |
| 1649 | −0.36535 | −0.34122 | 0 | 0.272545 | 0 | 0 | 9 | 535.9631 | 0.750061 |
| 1650 | −0.35247 | −0.33749 | 0 | 0 | 0.244058 | 0 | 9 | 534.633 | 0.749326 |
| 1651 | −0.3509 | −0.33426 | 0 | 0 | 0 | 0.230316 | 9 | 536.349 | 0.748367 |
| 1652 | −0.35435 | 0 | 0.326983 | 0 | 0.276248 | 0 | 9 | 544.6921 | 0.734097 |
| 1653 | −0.35429 | 0 | 0.335115 | 0 | 0 | 0.278056 | 9 | 546.2652 | 0.732627 |
| 1654 | −0.35918 | 0 | 0 | 0.257296 | 0.231108 | 0 | 9 | 543.5415 | 0.736057 |
| 1655 | −0.36046 | 0 | 0 | 0.27483 | 0 | 0.241127 | 9 | 544.2629 | 0.737874 |
| 1656 | −0.34604 | 0 | 0 | 0 | 0.20186 | 0.189915 | 9 | 539.8996 | 0.742732 |
| 1657 | −0.36484 | −0.51223 | 0.448932 | 0 | 0 | 0 | 9 | 529.0514 | 0.756839 |
| 1658 | −0.3635 | 0 | 0.280897 | 0.247949 | 0 | 0 | 9 | 545.2865 | 0.733239 |
| 1659 | −0.36535 | −0.34122 | 0 | 0.272545 | 0 | 0 | 9 | 531.5719 | 0.757431 |
| 1660 | −0.35247 | −0.33749 | 0 | 0 | 0.244058 | 0 | 9 | 530.037 | 0.756308 |
| 1661 | −0.3509 | −0.33426 | 0 | 0 | 0 | 0.230316 | 9 | 529.9291 | 0.757309 |
| 1662 | −0.35435 | 0 | 0.326983 | 0 | 0.276248 | 0 | 9 | 541.9185 | 0.737996 |
| 1663 | −0.35429 | 0 | 0.335115 | 0 | 0 | 0.278056 | 9 | 541.8064 | 0.739446 |
| 1664 | −0.35918 | 0 | 0 | 0.257296 | 0.231108 | 0 | 9 | 539.8867 | 0.741936 |
| 1665 | −0.36046 | 0 | 0 | 0.27483 | 0 | 0.241127 | 9 | 538.8945 | 0.74657 |
| 1666 | −0.37665 | −0.49016 | 0.415897 | 0.213769 | 0 | 0 | 9 | 530.0363 | 0.756676 |
| 1667 | −0.3692 | −0.49878 | 0.458935 | 0 | 0.257995 | 0 | 9 | 526.4908 | 0.757737 |
| 1668 | −0.36789 | −0.4958 | 0.464972 | 0 | 0 | 0.255069 | 9 | 528.5142 | 0.754797 |
| 1669 | −0.36683 | −0.32775 | 0 | 0.247282 | 0.215748 | 0 | 9 | 531.8412 | 0.755349 |
| 1670 | −0.36769 | −0.32552 | 0 | 0.266766 | 0 | 0.223296 | 9 | 532.8829 | 0.754716 |
| 1671 | −0.35346 | −0.32601 | 0 | 0 | 0.190682 | 0.171912 | 9 | 533.6129 | 0.751531 |
| 1672 | −0.36573 | 0 | 0.297911 | 0.217151 | 0.249518 | 0 | 9 | 542.5116 | 0.737465 |
| 1673 | −0.36751 | 0 | 0.304766 | 0.234822 | 0 | 0.26618 | 9 | 543.4877 | 0.737874 |
| 1674 | −0.35702 | 0 | 0.341819 | 0 | 0.21209 | 0.213821 | 9 | 542.7638 | 0.737363 |
| 1675 | −0.36116 | 0 | 0 | 0.256551 | 0.172681 | 0.188346 | 9 | 542.0122 | 0.740303 |
| 1676 | −0.34604 | 0 | 0 | 0 | 0.20186 | 0.189915 | 9 | 539.2674 | 0.742385 |
| 1677 | −0.36484 | −0.51223 | 0.448932 | 0 | 0 | 0 | 9 | 528.6327 | 0.756798 |
| 1678 | −0.3635 | 0 | 0.280897 | 0.247949 | 0 | 0 | 9 | 544.6351 | 0.734076 |
| 1679 | −0.36535 | −0.34122 | 0 | 0.272545 | 0 | 0 | 9 | 531.0309 | 0.757778 |
| 1680 | −0.35247 | −0.33749 | 0 | 0 | 0.244058 | 0 | 9 | 529.7626 | 0.756676 |
| 1681 | −0.3509 | −0.33426 | 0 | 0 | 0 | 0.230316 | 9 | 529.2006 | 0.758697 |
| 1682 | −0.35435 | 0 | 0.326983 | 0 | 0.276248 | 0 | 9 | 541.5774 | 0.736955 |
| 1683 | −0.35429 | 0 | 0.335115 | 0 | 0 | 0.278056 | 9 | 541.0115 | 0.739629 |
| 1684 | −0.35918 | 0 | 0 | 0.257296 | 0.231108 | 0 | 9 | 539.5213 | 0.742242 |
| 1685 | −0.36046 | 0 | 0 | 0.27483 | 0 | 0.241127 | 9 | 538.1313 | 0.746591 |
| 1686 | −0.37665 | −0.49016 | 0.415897 | 0.213769 | 0 | 0 | 9 | 530.118 | 0.757309 |
| 1687 | −0.3692 | −0.49878 | 0.458935 | 0 | 0.257995 | 0 | 9 | 527.2923 | 0.75737 |
| 1688 | −0.36789 | −0.4958 | 0.464972 | 0 | 0 | 0.255069 | 9 | 528.5776 | 0.756022 |
| 1689 | −0.36683 | −0.32775 | 0 | 0.247282 | 0.215748 | 0 | 9 | 532.3324 | 0.753777 |
| 1690 | −0.36769 | −0.32552 | 0 | 0.266766 | 0 | 0.223296 | 9 | 532.7938 | 0.755247 |
| 1691 | −0.35346 | −0.32601 | 0 | 0 | 0.190682 | 0.171912 | 9 | 533.8636 | 0.750817 |
| 1692 | −0.36573 | 0 | 0.297911 | 0.217151 | 0.249518 | 0 | 9 | 542.9044 | 0.736261 |
| 1693 | −0.36751 | 0 | 0.304766 | 0.234822 | 0 | 0.26618 | 9 | 543.3065 | 0.737404 |
| 1694 | −0.35702 | 0 | 0.341819 | 0 | 0.21209 | 0.213821 | 9 | 542.9922 | 0.735587 |
| 1695 | −0.36116 | 0 | 0 | 0.256551 | 0.172681 | 0.188346 | 9 | 542.1851 | 0.73818 |
| 1696 | −0.37665 | −0.49016 | 0.415897 | 0.213769 | 0 | 0 | 9 | 525.9534 | 0.762861 |
| 1697 | −0.3692 | −0.49878 | 0.458935 | 0 | 0.257995 | 0 | 9 | 522.6189 | 0.765393 |
| 1698 | −0.36789 | −0.4958 | 0.464972 | 0 | 0 | 0.255069 | 9 | 522.1259 | 0.764535 |
| 1699 | −0.36683 | −0.32775 | 0 | 0.247282 | 0.215748 | 0 | 9 | 526.6993 | 0.762351 |
| 1700 | −0.36769 | −0.32552 | 0 | 0.266766 | 0 | 0.223296 | 9 | 525.2915 | 0.766414 |
| 1701 | −0.35346 | −0.32601 | 0 | 0 | 0.190682 | 0.171912 | 9 | 526.7889 | 0.760146 |
| 1702 | −0.36573 | 0 | 0.297911 | 0.217151 | 0.249518 | 0 | 9 | 539.2817 | 0.74161 |
| 1703 | −0.36751 | 0 | 0.304766 | 0.234822 | 0 | 0.26618 | 9 | 538.0061 | 0.746019 |
| 1704 | −0.35702 | 0 | 0.341819 | 0 | 0.21209 | 0.213821 | 9 | 537.8807 | 0.743018 |
| 1705 | −0.36116 | 0 | 0 | 0.256551 | 0.172681 | 0.188346 | 9 | 536.1088 | 0.748734 |
| 1706 | −0.3784 | −0.48038 | 0.429094 | 0.184036 | 0.235336 | 0 | 9 | 525.8834 | 0.761841 |
| 1707 | −0.37944 | −0.47686 | 0.432955 | 0.204998 | 0 | 0.247828 | 9 | 526.6196 | 0.760759 |
| 1708 | −0.37057 | −0.48826 | 0.469023 | 0 | 0.198485 | 0.194003 | 9 | 525.8561 | 0.760105 |
| 1709 | −0.36821 | −0.31823 | 0 | 0.248938 | 0.16128 | 0.17389 | 9 | 531.3455 | 0.755614 |
| 1710 | −0.36844 | 0 | 0.31303 | 0.214129 | 0.186465 | 0.210249 | 9 | 541.157 | 0.739772 |
| 1711 | −0.34954 | 0 | 0.311734 | 0 | 0 | 0 | 9 | 552.8294 | 0.718275 |
| 1712 | −0.34838 | −0.35299 | 0 | 0 | 0 | 0 | 9 | 539.8827 | 0.742977 |
| 1713 | −0.35731 | 0 | 0 | 0.283735 | 0 | 0 | 9 | 548.7966 | 0.728442 |
| 1714 | −0.34421 | 0 | 0 | 0 | 0.260301 | 0 | 9 | 547.3427 | 0.728319 |
| 1715 | −0.34349 | 0 | 0 | 0 | 0 | 0.25144 | 9 | 547.0371 | 0.728769 |
| 1716 | −0.34604 | 0 | 0 | 0 | 0.20186 | 0.189915 | 9 | 549.1417 | 0.725951 |
| 1717 | −0.36484 | −0.51223 | 0.448932 | 0 | 0 | 0 | 9 | 537.4037 | 0.743753 |
| 1718 | −0.3635 | 0 | 0.280897 | 0.247949 | 0 | 0 | 9 | 552.0119 | 0.720235 |
| 1719 | −0.36535 | −0.34122 | 0 | 0.272545 | 0 | 0 | 9 | 539.8686 | 0.742875 |
| 1720 | −0.35247 | −0.33749 | 0 | 0 | 0.244058 | 0 | 9 | 539.0962 | 0.742365 |
| 1721 | −0.3509 | −0.33426 | 0 | 0 | 0 | 0.230316 | 9 | 540.5161 | 0.741058 |
| 1722 | −0.35435 | 0 | 0.326983 | 0 | 0.276248 | 0 | 9 | 549.6013 | 0.722562 |
| 1723 | −0.35429 | 0 | 0.335115 | 0 | 0 | 0.278056 | 9 | 550.8074 | 0.720786 |
| 1724 | −0.35918 | 0 | 0 | 0.257296 | 0.231108 | 0 | 9 | 547.5909 | 0.728279 |
| 1725 | −0.36046 | 0 | 0 | 0.27483 | 0 | 0.241127 | 9 | 548.0507 | 0.729034 |
| 1726 | −0.34604 | 0 | 0 | 0 | 0.20186 | 0.189915 | 9 | 543.9682 | 0.733791 |
| 1727 | −0.36484 | −0.51223 | 0.448932 | 0 | 0 | 0 | 9 | 533.8412 | 0.750592 |
| 1728 | −0.3635 | 0 | 0.280897 | 0.247949 | 0 | 0 | 9 | 549.0675 | 0.726748 |
| 1729 | −0.36535 | −0.34122 | 0 | 0.272545 | 0 | 0 | 9 | 535.4217 | 0.751164 |
| 1730 | −0.35247 | −0.33749 | 0 | 0 | 0.244058 | 0 | 9 | 534.387 | 0.749898 |
| 1731 | −0.3509 | −0.33426 | 0 | 0 | 0 | 0.230316 | 9 | 534.1775 | 0.751307 |
| 1732 | −0.35435 | 0 | 0.326983 | 0 | 0.276248 | 0 | 9 | 546.4255 | 0.729626 |
| 1733 | −0.35429 | 0 | 0.335115 | 0 | 0 | 0.278056 | 9 | 546.1433 | 0.72983 |
| 1734 | −0.35918 | 0 | 0 | 0.257296 | 0.231108 | 0 | 9 | 543.675 | 0.7362 |
| 1735 | −0.36046 | 0 | 0 | 0.27483 | 0 | 0.241127 | 9 | 542.5956 | 0.737976 |
| 1736 | −0.37665 | −0.49016 | 0.415897 | 0.213769 | 0 | 0 | 9 | 534.4957 | 0.750653 |
| 1737 | −0.3692 | −0.49878 | 0.458935 | 0 | 0.257995 | 0 | 9 | 531.751 | 0.751102 |
| 1738 | −0.36789 | −0.4958 | 0.464972 | 0 | 0 | 0.255069 | 9 | 533.3148 | 0.748918 |
| 1739 | −0.36683 | −0.32775 | 0 | 0.247282 | 0.215748 | 0 | 9 | 535.7772 | 0.750347 |
| 1740 | −0.36769 | −0.32552 | 0 | 0.266766 | 0 | 0.223296 | 9 | 536.4942 | 0.749041 |
| 1741 | −0.35346 | −0.32601 | 0 | 0 | 0.190682 | 0.171912 | 9 | 537.7024 | 0.746121 |
| 1742 | −0.36573 | 0 | 0.297911 | 0.217151 | 0.249518 | 0 | 9 | 546.8197 | 0.729708 |
| 1743 | −0.36751 | 0 | 0.304766 | 0.234822 | 0 | 0.26618 | 9 | 547.4198 | 0.729197 |
| 1744 | −0.35702 | 0 | 0.341819 | 0 | 0.21209 | 0.213821 | 9 | 547.335 | 0.726768 |
| 1745 | −0.36116 | 0 | 0 | 0.256551 | 0.172681 | 0.188346 | 9 | 545.6589 | 0.733668 |
| 1746 | −0.34604 | 0 | 0 | 0 | 0.20186 | 0.189915 | 9 | 544.1504 | 0.731912 |
| 1747 | −0.36484 | −0.51223 | 0.448932 | 0 | 0 | 0 | 9 | 533.9776 | 0.750429 |
| 1748 | −0.3635 | 0 | 0.280897 | 0.247949 | 0 | 0 | 9 | 549.0011 | 0.725911 |
| 1749 | −0.36535 | −0.34122 | 0 | 0.272545 | 0 | 0 | 9 | 535.4229 | 0.751286 |
| 1750 | −0.35247 | −0.33749 | 0 | 0 | 0.244058 | 0 | 9 | 534.8876 | 0.748857 |
| 1751 | −0.3509 | −0.33426 | 0 | 0 | 0 | 0.230316 | 9 | 534.0516 | 0.751511 |
| 1752 | −0.35435 | 0 | 0.326983 | 0 | 0.276248 | 0 | 9 | 546.8878 | 0.726115 |
| 1753 | −0.35429 | 0 | 0.335115 | 0 | 0 | 0.278056 | 9 | 546.0165 | 0.727911 |
| 1754 | −0.35918 | 0 | 0 | 0.257296 | 0.231108 | 0 | 9 | 544.0662 | 0.733995 |
| 1755 | −0.36046 | 0 | 0 | 0.27483 | 0 | 0.241127 | 9 | 542.4673 | 0.737363 |
| 1756 | −0.37665 | −0.49016 | 0.415897 | 0.213769 | 0 | 0 | 9 | 535.257 | 0.749653 |
| 1757 | −0.3692 | −0.49878 | 0.458935 | 0 | 0.257995 | 0 | 9 | 533.3873 | 0.748816 |
| 1758 | −0.36789 | −0.4958 | 0.464972 | 0 | 0 | 0.255069 | 9 | 534.1352 | 0.747816 |
| 1759 | −0.36683 | −0.32775 | 0 | 0.247282 | 0.215748 | 0 | 9 | 537.0517 | 0.746448 |
| 1760 | −0.36769 | −0.32552 | 0 | 0.266766 | 0 | 0.223296 | 9 | 537.1158 | 0.747856 |
| 1761 | −0.35346 | −0.32601 | 0 | 0 | 0.190682 | 0.171912 | 9 | 538.7949 | 0.742528 |
| 1762 | −0.36573 | 0 | 0.297911 | 0.217151 | 0.249518 | 0 | 9 | 548.0251 | 0.726645 |
| 1763 | −0.36751 | 0 | 0.304766 | 0.234822 | 0 | 0.26618 | 9 | 547.9814 | 0.727687 |
| 1764 | −0.35702 | 0 | 0.341819 | 0 | 0.21209 | 0.213821 | 9 | 548.4215 | 0.723277 |
| 1765 | −0.36116 | 0 | 0 | 0.256551 | 0.172681 | 0.188346 | 9 | 546.6248 | 0.729238 |
| 1766 | −0.37665 | −0.49016 | 0.415897 | 0.213769 | 0 | 0 | 9 | 530.9573 | 0.756247 |
| 1767 | −0.3692 | −0.49878 | 0.458935 | 0 | 0.257995 | 0 | 9 | 528.6142 | 0.756002 |
| 1768 | −0.36789 | −0.4958 | 0.464972 | 0 | 0 | 0.255069 | 9 | 527.74 | 0.757635 |
| 1769 | −0.36683 | −0.32775 | 0 | 0.247282 | 0.215748 | 0 | 9 | 531.3904 | 0.755941 |
| 1770 | −0.36769 | −0.32552 | 0 | 0.266766 | 0 | 0.223296 | 9 | 529.7512 | 0.759962 |
| 1771 | −0.35346 | −0.32601 | 0 | 0 | 0.190682 | 0.171912 | 9 | 531.7634 | 0.753205 |
| 1772 | −0.36573 | 0 | 0.297911 | 0.217151 | 0.249518 | 0 | 9 | 544.043 | 0.73326 |
| 1773 | −0.36751 | 0 | 0.304766 | 0.234822 | 0 | 0.26618 | 9 | 542.4899 | 0.736935 |
| 1774 | −0.35702 | 0 | 0.341819 | 0 | 0.21209 | 0.213821 | 9 | 543.0621 | 0.732055 |
| 1775 | −0.36116 | 0 | 0 | 0.256551 | 0.172681 | 0.188346 | 9 | 540.4459 | 0.739507 |
| 1776 | −0.3784 | −0.48038 | 0.429094 | 0.184036 | 0.235336 | 0 | 9 | 531.3548 | 0.752205 |
| 1777 | −0.37944 | −0.47686 | 0.432955 | 0.204998 | 0 | 0.247828 | 9 | 531.5515 | 0.752205 |
| 1778 | −0.37057 | −0.48826 | 0.469023 | 0 | 0.198485 | 0.194003 | 9 | 531.5529 | 0.750429 |
| 1779 | −0.36821 | −0.31823 | 0 | 0.248938 | 0.16128 | 0.17389 | 9 | 535.5391 | 0.749592 |
| 1780 | −0.36844 | 0 | 0.31303 | 0.214129 | 0.186465 | 0.210249 | 9 | 545.7805 | 0.730871 |
| 1781 | −0.34604 | 0 | 0 | 0 | 0.20186 | 0.189915 | 9 | 544.7286 | 0.733893 |
| 1782 | −0.36484 | −0.51223 | 0.448932 | 0 | 0 | 0 | 9 | 535.2206 | 0.748448 |
| 1783 | −0.3635 | 0 | 0.280897 | 0.247949 | 0 | 0 | 9 | 549.8796 | 0.724257 |
| 1784 | −0.36535 | −0.34122 | 0 | 0.272545 | 0 | 0 | 9 | 536.3008 | 0.750388 |
| 1785 | −0.35247 | −0.33749 | 0 | 0 | 0.244058 | 0 | 9 | 535.5706 | 0.748163 |
| 1786 | −0.3509 | −0.33426 | 0 | 0 | 0 | 0.230316 | 9 | 535.0722 | 0.749 |
| 1787 | −0.35435 | 0 | 0.326983 | 0 | 0.276248 | 0 | 9 | 547.4767 | 0.727176 |
| 1788 | −0.35429 | 0 | 0.335115 | 0 | 0 | 0.278056 | 9 | 546.908 | 0.728544 |
| 1789 | −0.35918 | 0 | 0 | 0.257296 | 0.231108 | 0 | 9 | 544.4077 | 0.734628 |
| 1790 | −0.36046 | 0 | 0 | 0.27483 | 0 | 0.241127 | 9 | 543.0233 | 0.737874 |
| 1791 | −0.37665 | −0.49016 | 0.415897 | 0.213769 | 0 | 0 | 9 | 535.0617 | 0.748755 |
| 1792 | −0.3692 | −0.49878 | 0.458935 | 0 | 0.257995 | 0 | 9 | 532.4402 | 0.750102 |
| 1793 | −0.36789 | −0.4958 | 0.464972 | 0 | 0 | 0.255069 | 9 | 533.674 | 0.749081 |
| 1794 | −0.36683 | −0.32775 | 0 | 0.247282 | 0.215748 | 0 | 9 | 536.0107 | 0.74902 |
| 1795 | −0.36769 | −0.32552 | 0 | 0.266766 | 0 | 0.223296 | 9 | 536.4181 | 0.748734 |
| 1796 | −0.35346 | −0.32601 | 0 | 0 | 0.190682 | 0.171912 | 9 | 537.8149 | 0.744508 |
| 1797 | −0.36573 | 0 | 0.297911 | 0.217151 | 0.249518 | 0 | 9 | 547.0935 | 0.728973 |
| 1798 | −0.36751 | 0 | 0.304766 | 0.234822 | 0 | 0.26618 | 9 | 547.376 | 0.729463 |
| 1799 | −0.35702 | 0 | 0.341819 | 0 | 0.21209 | 0.213821 | 9 | 547.4635 | 0.726298 |
| 1800 | −0.36116 | 0 | 0 | 0.256551 | 0.172681 | 0.188346 | 9 | 545.5063 | 0.732607 |
| 1801 | −0.37665 | −0.49016 | 0.415897 | 0.213769 | 0 | 0 | 9 | 531.6931 | 0.754042 |
| 1802 | −0.3692 | −0.49878 | 0.458935 | 0 | 0.257995 | 0 | 9 | 528.9643 | 0.75543 |
| 1803 | −0.36789 | −0.4958 | 0.464972 | 0 | 0 | 0.255069 | 9 | 528.4811 | 0.755818 |
| 1804 | −0.36683 | −0.32775 | 0 | 0.247282 | 0.215748 | 0 | 9 | 531.5504 | 0.756002 |
| 1805 | −0.36769 | −0.32552 | 0 | 0.266766 | 0 | 0.223296 | 9 | 530.1534 | 0.759207 |
| 1806 | −0.35346 | −0.32601 | 0 | 0 | 0.190682 | 0.171912 | 9 | 532.1731 | 0.753042 |
| 1807 | −0.36573 | 0 | 0.297911 | 0.217151 | 0.249518 | 0 | 9 | 544.1646 | 0.733219 |
| 1808 | −0.36751 | 0 | 0.304766 | 0.234822 | 0 | 0.26618 | 9 | 542.861 | 0.7372 |
| 1809 | −0.35702 | 0 | 0.341819 | 0 | 0.21209 | 0.213821 | 9 | 543.3302 | 0.733403 |
| 1810 | −0.36116 | 0 | 0 | 0.256551 | 0.172681 | 0.188346 | 9 | 540.4829 | 0.739997 |
| 1811 | −0.3784 | −0.48038 | 0.429094 | 0.184036 | 0.235336 | 0 | 9 | 530.0727 | 0.754695 |
| 1812 | −0.37944 | −0.47686 | 0.432955 | 0.204998 | 0 | 0.247828 | 9 | 530.6798 | 0.752981 |
| 1813 | −0.37057 | −0.48826 | 0.469023 | 0 | 0.198485 | 0.194003 | 9 | 530.2442 | 0.75296 |
| 1814 | −0.36821 | −0.31823 | 0 | 0.248938 | 0.16128 | 0.17389 | 9 | 534.1605 | 0.751776 |
| 1815 | −0.36844 | 0 | 0.31303 | 0.214129 | 0.186465 | 0.210249 | 9 | 544.4712 | 0.733444 |
| 1816 | −0.37665 | −0.49016 | 0.415897 | 0.213769 | 0 | 0 | 9 | 531.8421 | 0.75394 |
| 1817 | −0.3692 | −0.49878 | 0.458935 | 0 | 0.257995 | 0 | 9 | 529.5711 | 0.753756 |
| 1818 | −0.36789 | −0.4958 | 0.464972 | 0 | 0 | 0.255069 | 9 | 528.574 | 0.755941 |
| 1819 | −0.36683 | −0.32775 | 0 | 0.247282 | 0.215748 | 0 | 9 | 531.9923 | 0.755226 |
| 1820 | −0.36769 | −0.32552 | 0 | 0.266766 | 0 | 0.223296 | 9 | 530.1627 | 0.759064 |
| 1821 | −0.35346 | −0.32601 | 0 | 0 | 0.190682 | 0.171912 | 9 | 532.4175 | 0.752409 |
| 1822 | −0.36573 | 0 | 0.297911 | 0.217151 | 0.249518 | 0 | 9 | 544.5689 | 0.731647 |
| 1823 | −0.36751 | 0 | 0.304766 | 0.234822 | 0 | 0.26618 | 9 | 542.8358 | 0.73573 |
| 1824 | −0.35702 | 0 | 0.341819 | 0 | 0.21209 | 0.213821 | 9 | 543.5801 | 0.731565 |
| 1825 | −0.36116 | 0 | 0 | 0.256551 | 0.172681 | 0.188346 | 9 | 540.6952 | 0.738139 |
| 1826 | −0.3784 | −0.48038 | 0.429094 | 0.184036 | 0.235336 | 0 | 9 | 531.4837 | 0.751449 |
| 1827 | −0.37944 | −0.47686 | 0.432955 | 0.204998 | 0 | 0.247828 | 9 | 531.5042 | 0.752225 |
| 1828 | −0.37057 | −0.48826 | 0.469023 | 0 | 0.198485 | 0.194003 | 9 | 531.5668 | 0.750674 |
| 1829 | −0.36821 | −0.31823 | 0 | 0.248938 | 0.16128 | 0.17389 | 9 | 535.2524 | 0.749551 |
| 1830 | −0.36844 | 0 | 0.31303 | 0.214129 | 0.186465 | 0.210249 | 9 | 545.5158 | 0.730851 |
| 1831 | −0.3784 | −0.48038 | 0.429094 | 0.184036 | 0.235336 | 0 | 9 | 527.0514 | 0.759493 |
| 1832 | −0.37944 | −0.47686 | 0.432955 | 0.204998 | 0 | 0.247828 | 9 | 525.3107 | 0.763494 |
| 1833 | −0.37057 | −0.48826 | 0.469023 | 0 | 0.198485 | 0.194003 | 9 | 525.7113 | 0.759881 |
| 1834 | −0.36821 | −0.31823 | 0 | 0.248938 | 0.16128 | 0.17389 | 9 | 528.3103 | 0.760105 |
| 1835 | −0.36844 | 0 | 0.31303 | 0.214129 | 0.186465 | 0.210249 | 9 | 540.3433 | 0.739058 |
| 1836 | −0.38017 | −0.47149 | 0.43946 | 0.184707 | 0.175726 | 0.194494 | 9 | 529.3619 | 0.754634 |
| 1837 | −0.34954 | 0 | 0.311734 | 0 | 0 | 0 | 9 | 578.3621 | 0.671301 |
| 1838 | −0.34838 | −0.35299 | 0 | 0 | 0 | 0 | 9 | 578.1699 | 0.670709 |
| 1839 | −0.35731 | 0 | 0 | 0.283735 | 0 | 0 | 9 | 580.4341 | 0.66881 |
| 1840 | −0.34421 | 0 | 0 | 0 | 0.260301 | 0 | 9 | 579.4286 | 0.67371 |
| 1841 | −0.34349 | 0 | 0 | 0 | 0 | 0.25144 | 9 | 579.1404 | 0.669484 |
| 1842 | −0.34604 | 0 | 0 | 0 | 0.20186 | 0.189915 | 9 | 580.2193 | 0.673301 |
| 1843 | −0.36484 | −0.51223 | 0.448932 | 0 | 0 | 0 | 9 | 564.3958 | 0.695084 |
| 1844 | −0.3635 | 0 | 0.280897 | 0.247949 | 0 | 0 | 9 | 576.0567 | 0.676057 |
| 1845 | −0.36535 | −0.34122 | 0 | 0.272545 | 0 | 0 | 9 | 575.7555 | 0.67571 |
| 1846 | −0.35247 | −0.33749 | 0 | 0 | 0.244058 | 0 | 9 | 575.4056 | 0.682631 |
| 1847 | −0.3509 | −0.33426 | 0 | 0 | 0 | 0.230316 | 9 | 576.9523 | 0.673853 |
| 1848 | −0.35435 | 0 | 0.326983 | 0 | 0.276248 | 0 | 9 | 572.4965 | 0.683795 |
| 1849 | −0.35429 | 0 | 0.335115 | 0 | 0 | 0.278056 | 9 | 573.8612 | 0.682896 |
| 1850 | −0.35918 | 0 | 0 | 0.257296 | 0.231108 | 0 | 9 | 577.733 | 0.676098 |
| 1851 | −0.36046 | 0 | 0 | 0.27483 | 0 | 0.241127 | 9 | 578.2281 | 0.67077 |
| 1852 | −0.34604 | 0 | 0 | 0 | 0.20186 | 0.189915 | 9 | 575.9336 | 0.675486 |
| 1853 | −0.36484 | −0.51223 | 0.448932 | 0 | 0 | 0 | 9 | 561.9883 | 0.699453 |
| 1854 | −0.3635 | 0 | 0.280897 | 0.247949 | 0 | 0 | 9 | 574.2358 | 0.679977 |
| 1855 | −0.36535 | −0.34122 | 0 | 0.272545 | 0 | 0 | 9 | 572.7657 | 0.683591 |
| 1856 | −0.35247 | −0.33749 | 0 | 0 | 0.244058 | 0 | 9 | 572.0598 | 0.684366 |
| 1857 | −0.3509 | −0.33426 | 0 | 0 | 0 | 0.230316 | 9 | 572.0892 | 0.681447 |
| 1858 | −0.35435 | 0 | 0.326983 | 0 | 0.276248 | 0 | 9 | 570.262 | 0.684611 |
| 1859 | −0.35429 | 0 | 0.335115 | 0 | 0 | 0.278056 | 9 | 569.804 | 0.686081 |
| 1860 | −0.35918 | 0 | 0 | 0.257296 | 0.231108 | 0 | 9 | 574.8626 | 0.680345 |
| 1861 | −0.36046 | 0 | 0 | 0.27483 | 0 | 0.241127 | 9 | 573.7234 | 0.679916 |
| 1862 | −0.37665 | −0.49016 | 0.415897 | 0.213769 | 0 | 0 | 9 | 561.5682 | 0.698228 |
| 1863 | −0.3692 | −0.49878 | 0.458935 | 0 | 0.257995 | 0 | 9 | 557.5735 | 0.704536 |
| 1864 | −0.36789 | −0.4958 | 0.464972 | 0 | 0 | 0.255069 | 9 | 559.2648 | 0.700882 |
| 1865 | −0.36683 | −0.32775 | 0 | 0.247282 | 0.215748 | 0 | 9 | 571.3839 | 0.686734 |
| 1866 | −0.36769 | −0.32552 | 0 | 0.266766 | 0 | 0.223296 | 9 | 572.0525 | 0.678711 |
| 1867 | −0.35346 | −0.32601 | 0 | 0 | 0.190682 | 0.171912 | 9 | 574.0317 | 0.679671 |
| 1868 | −0.36573 | 0 | 0.297911 | 0.217151 | 0.249518 | 0 | 9 | 569.7766 | 0.691246 |
| 1869 | −0.36751 | 0 | 0.304766 | 0.234822 | 0 | 0.26618 | 9 | 570.3978 | 0.68555 |
| 1870 | −0.35702 | 0 | 0.341819 | 0 | 0.21209 | 0.213821 | 9 | 569.7717 | 0.688082 |
| 1871 | −0.36116 | 0 | 0 | 0.256551 | 0.172681 | 0.188346 | 9 | 575.9281 | 0.676568 |
| 1872 | −0.34604 | 0 | 0 | 0 | 0.20186 | 0.189915 | 9 | 575.4852 | 0.67816 |
| 1873 | −0.36484 | −0.51223 | 0.448932 | 0 | 0 | 0 | 9 | 561.718 | 0.701474 |
| 1874 | −0.3635 | 0 | 0.280897 | 0.247949 | 0 | 0 | 9 | 573.6692 | 0.680671 |
| 1875 | −0.36535 | −0.34122 | 0 | 0.272545 | 0 | 0 | 9 | 572.1483 | 0.684856 |
| 1876 | −0.35247 | −0.33749 | 0 | 0 | 0.244058 | 0 | 9 | 571.891 | 0.687388 |
| 1877 | −0.3509 | −0.33426 | 0 | 0 | 0 | 0.230316 | 9 | 571.3438 | 0.684877 |
| 1878 | −0.35435 | 0 | 0.326983 | 0 | 0.276248 | 0 | 9 | 570.3735 | 0.683815 |
| 1879 | −0.35429 | 0 | 0.335115 | 0 | 0 | 0.278056 | 9 | 569.1452 | 0.688266 |
| 1880 | −0.35918 | 0 | 0 | 0.257296 | 0.231108 | 0 | 9 | 574.6438 | 0.680385 |
| 1881 | −0.36046 | 0 | 0 | 0.27483 | 0 | 0.241127 | 9 | 572.922 | 0.681529 |
| 1882 | −0.37665 | −0.49016 | 0.415897 | 0.213769 | 0 | 0 | 9 | 561.658 | 0.700024 |
| 1883 | −0.3692 | −0.49878 | 0.458935 | 0 | 0.257995 | 0 | 9 | 558.6189 | 0.704352 |
| 1884 | −0.36789 | −0.4958 | 0.464972 | 0 | 0 | 0.255069 | 9 | 559.4282 | 0.701903 |
| 1885 | −0.36683 | −0.32775 | 0 | 0.247282 | 0.215748 | 0 | 9 | 571.6896 | 0.685448 |
| 1886 | −0.36769 | −0.32552 | 0 | 0.266766 | 0 | 0.223296 | 9 | 571.7509 | 0.68061 |
| 1887 | −0.35346 | −0.32601 | 0 | 0 | 0.190682 | 0.171912 | 9 | 574.1499 | 0.681263 |
| 1888 | −0.36573 | 0 | 0.297911 | 0.217151 | 0.249518 | 0 | 9 | 570.4087 | 0.686755 |
| 1889 | −0.36751 | 0 | 0.304766 | 0.234822 | 0 | 0.26618 | 9 | 570.2506 | 0.686489 |
| 1890 | −0.35702 | 0 | 0.341819 | 0 | 0.21209 | 0.213821 | 9 | 570.289 | 0.686081 |
| 1891 | −0.36116 | 0 | 0 | 0.256551 | 0.172681 | 0.188346 | 9 | 576.0395 | 0.673853 |
| 1892 | −0.37665 | −0.49016 | 0.415897 | 0.213769 | 0 | 0 | 9 | 558.7912 | 0.706231 |
| 1893 | −0.3692 | −0.49878 | 0.458935 | 0 | 0.257995 | 0 | 9 | 555.1044 | 0.709701 |
| 1894 | −0.36789 | −0.4958 | 0.464972 | 0 | 0 | 0.255069 | 9 | 554.2127 | 0.711028 |
| 1895 | −0.36683 | −0.32775 | 0 | 0.247282 | 0.215748 | 0 | 9 | 567.7349 | 0.692124 |
| 1896 | −0.36769 | −0.32552 | 0 | 0.266766 | 0 | 0.223296 | 9 | 566.2333 | 0.691879 |
| 1897 | −0.35346 | −0.32601 | 0 | 0 | 0.190682 | 0.171912 | 9 | 568.8394 | 0.688021 |
| 1898 | −0.36573 | 0 | 0.297911 | 0.217151 | 0.249518 | 0 | 9 | 567.5489 | 0.69043 |
| 1899 | −0.36751 | 0 | 0.304766 | 0.234822 | 0 | 0.26618 | 9 | 565.6284 | 0.693573 |
| 1900 | −0.35702 | 0 | 0.341819 | 0 | 0.21209 | 0.213821 | 9 | 565.6787 | 0.690103 |
| 1901 | −0.36116 | 0 | 0 | 0.256551 | 0.172681 | 0.188346 | 9 | 571.0124 | 0.682162 |
| 1902 | −0.3784 | −0.48038 | 0.429094 | 0.184036 | 0.235336 | 0 | 9 | 556.8559 | 0.70619 |
| 1903 | −0.37944 | −0.47686 | 0.432955 | 0.204998 | 0 | 0.247828 | 9 | 557.0128 | 0.704148 |
| 1904 | −0.37057 | −0.48826 | 0.469023 | 0 | 0.198485 | 0.194003 | 9 | 556.3809 | 0.70425 |
| 1905 | −0.36821 | −0.31823 | 0 | 0.248938 | 0.16128 | 0.17389 | 9 | 570.2612 | 0.683427 |
| 1906 | −0.36844 | 0 | 0.31303 | 0.214129 | 0.186465 | 0.210249 | 9 | 567.7707 | 0.68898 |
| 1907 | −0.34604 | 0 | 0 | 0 | 0.20186 | 0.189915 | 9 | 575.7062 | 0.679099 |
| 1908 | −0.36484 | −0.51223 | 0.448932 | 0 | 0 | 0 | 9 | 562.733 | 0.69733 |
| 1909 | −0.36535 | −0.34122 | 0 | 0.272545 | 0 | 0 | 9 | 572.8163 | 0.684183 |
| 1910 | −0.3635 | 0 | 0.280897 | 0.247949 | 0 | 0 | 9 | 574.2554 | 0.679385 |
| 1911 | −0.35247 | −0.33749 | 0 | 0 | 0.244058 | 0 | 9 | 572.2859 | 0.687633 |
| 1912 | −0.3509 | −0.33426 | 0 | 0 | 0 | 0.230316 | 9 | 572.0886 | 0.683264 |
| 1913 | −0.35435 | 0 | 0.326983 | 0 | 0.276248 | 0 | 9 | 570.4849 | 0.68453 |
| 1914 | −0.35429 | 0 | 0.335115 | 0 | 0 | 0.278056 | 9 | 569.7024 | 0.689082 |
| 1915 | −0.35918 | 0 | 0 | 0.257296 | 0.231108 | 0 | 9 | 574.6792 | 0.680692 |
| 1916 | −0.36046 | 0 | 0 | 0.27483 | 0 | 0.241127 | 9 | 573.2424 | 0.682019 |
| 1917 | −0.37665 | −0.49016 | 0.415897 | 0.213769 | 0 | 0 | 9 | 561.5913 | 0.700555 |
| 1918 | −0.3692 | −0.49878 | 0.458935 | 0 | 0.257995 | 0 | 9 | 557.7048 | 0.706149 |
| 1919 | −0.36789 | −0.4958 | 0.464972 | 0 | 0 | 0.255069 | 9 | 559.0983 | 0.702964 |
| 1920 | −0.36683 | −0.32775 | 0 | 0.247282 | 0.215748 | 0 | 9 | 570.8572 | 0.68947 |
| 1921 | −0.36769 | −0.32552 | 0 | 0.266766 | 0 | 0.223296 | 9 | 571.2875 | 0.682243 |
| 1922 | −0.35346 | −0.32601 | 0 | 0 | 0.190682 | 0.171912 | 9 | 573.4148 | 0.684121 |
| 1923 | −0.36573 | 0 | 0.297911 | 0.217151 | 0.249518 | 0 | 9 | 569.3876 | 0.689164 |
| 1924 | −0.36751 | 0 | 0.304766 | 0.234822 | 0 | 0.26618 | 9 | 569.6865 | 0.688286 |
| 1925 | −0.35702 | 0 | 0.341819 | 0 | 0.21209 | 0.213821 | 9 | 569.2326 | 0.690225 |
| 1926 | −0.36116 | 0 | 0 | 0.256551 | 0.172681 | 0.188346 | 9 | 575.0325 | 0.678283 |
| 1927 | −0.37665 | −0.49016 | 0.415897 | 0.213769 | 0 | 0 | 9 | 559.3078 | 0.706067 |
| 1928 | −0.3692 | −0.49878 | 0.458935 | 0 | 0.257995 | 0 | 9 | 555.0858 | 0.710538 |
| 1929 | −0.36789 | −0.4958 | 0.464972 | 0 | 0 | 0.255069 | 9 | 554.7173 | 0.71015 |
| 1930 | −0.36683 | −0.32775 | 0 | 0.247282 | 0.215748 | 0 | 9 | 567.656 | 0.694553 |
| 1931 | −0.36769 | −0.32552 | 0 | 0.266766 | 0 | 0.223296 | 9 | 566.4456 | 0.692471 |
| 1932 | −0.35346 | −0.32601 | 0 | 0 | 0.190682 | 0.171912 | 9 | 568.963 | 0.689776 |
| 1933 | −0.36573 | 0 | 0.297911 | 0.217151 | 0.249518 | 0 | 9 | 567.2696 | 0.690491 |
| 1934 | −0.36751 | 0 | 0.304766 | 0.234822 | 0 | 0.26618 | 9 | 565.7387 | 0.693002 |
| 1935 | −0.35702 | 0 | 0.341819 | 0 | 0.21209 | 0.213821 | 9 | 565.5155 | 0.69241 |
| 1936 | −0.36116 | 0 | 0 | 0.256551 | 0.172681 | 0.188346 | 9 | 570.7835 | 0.684489 |
| 1937 | −0.3784 | −0.48038 | 0.429094 | 0.184036 | 0.235336 | 0 | 9 | 555.6201 | 0.70913 |
| 1938 | −0.37944 | −0.47686 | 0.432955 | 0.204998 | 0 | 0.247828 | 9 | 556.29 | 0.706802 |
| 1939 | −0.37057 | −0.48826 | 0.469023 | 0 | 0.198485 | 0.194003 | 9 | 555.1368 | 0.707721 |
| 1940 | −0.36821 | −0.31823 | 0 | 0.248938 | 0.16128 | 0.17389 | 9 | 569.1677 | 0.687163 |
| 1941 | −0.36844 | 0 | 0.31303 | 0.214129 | 0.186465 | 0.210249 | 9 | 566.4455 | 0.693451 |
| 1942 | −0.37665 | −0.49016 | 0.415897 | 0.213769 | 0 | 0 | 9 | 559.0861 | 0.707108 |
| 1943 | −0.3692 | −0.49878 | 0.458935 | 0 | 0.257995 | 0 | 9 | 555.4084 | 0.709987 |
| 1944 | −0.36789 | −0.4958 | 0.464972 | 0 | 0 | 0.255069 | 9 | 554.4623 | 0.710701 |
| 1945 | −0.36683 | −0.32775 | 0 | 0.247282 | 0.215748 | 0 | 9 | 567.4838 | 0.695554 |
| 1946 | −0.36769 | −0.32552 | 0 | 0.266766 | 0 | 0.223296 | 9 | 565.8462 | 0.69439 |
| 1947 | −0.35346 | −0.32601 | 0 | 0 | 0.190682 | 0.171912 | 9 | 568.6099 | 0.69243 |
| 1948 | −0.36573 | 0 | 0.297911 | 0.217151 | 0.249518 | 0 | 9 | 567.3021 | 0.689613 |
| 1949 | −0.36751 | 0 | 0.304766 | 0.234822 | 0 | 0.26618 | 9 | 565.1959 | 0.695554 |
| 1950 | −0.35702 | 0 | 0.341819 | 0 | 0.21209 | 0.213821 | 9 | 565.3807 | 0.693431 |
| 1951 | −0.36116 | 0 | 0 | 0.256551 | 0.172681 | 0.188346 | 9 | 570.3786 | 0.68602 |
| 1952 | −0.3784 | −0.48038 | 0.429094 | 0.184036 | 0.235336 | 0 | 9 | 556.5296 | 0.707925 |
| 1953 | −0.37944 | −0.47686 | 0.432955 | 0.204998 | 0 | 0.247828 | 9 | 556.5277 | 0.705965 |
| 1954 | −0.37057 | −0.48826 | 0.469023 | 0 | 0.198485 | 0.194003 | 9 | 555.9863 | 0.706047 |
| 1955 | −0.36821 | −0.31823 | 0 | 0.248938 | 0.16128 | 0.17389 | 9 | 569.3862 | 0.686387 |
| 1956 | −0.36844 | 0 | 0.31303 | 0.214129 | 0.186465 | 0.210249 | 9 | 566.9951 | 0.690715 |
| 1957 | −0.3784 | −0.48038 | 0.429094 | 0.184036 | 0.235336 | 0 | 9 | 553.2001 | 0.713192 |
| 1958 | −0.37944 | −0.47686 | 0.432955 | 0.204998 | 0 | 0.247828 | 9 | 551.4439 | 0.713886 |
| 1959 | −0.37057 | −0.48826 | 0.469023 | 0 | 0.198485 | 0.194003 | 9 | 550.9953 | 0.712967 |
| 1960 | −0.36821 | −0.31823 | 0 | 0.248938 | 0.16128 | 0.17389 | 9 | 564.0021 | 0.696574 |
| 1961 | −0.36844 | 0 | 0.31303 | 0.214129 | 0.186465 | 0.210249 | 9 | 562.3982 | 0.697105 |
| 1962 | −0.38017 | −0.47149 | 0.43946 | 0.184707 | 0.175726 | 0.194494 | 9 | 554.1284 | 0.707211 |
| 1963 | −0.34604 | 0 | 0 | 0 | 0.20186 | 0.189915 | 9 | 576.752 | 0.678058 |
| 1964 | −0.36484 | −0.51223 | 0.448932 | 0 | 0 | 0 | 9 | 563.97 | 0.700535 |
| 1965 | −0.36535 | −0.34122 | 0 | 0.272545 | 0 | 0 | 9 | 573.3431 | 0.685734 |
| 1966 | −0.3635 | 0 | 0.280897 | 0.247949 | 0 | 0 | 9 | 575.5629 | 0.678466 |
| 1967 | −0.35247 | −0.33749 | 0 | 0 | 0.244058 | 0 | 9 | 573.0067 | 0.687919 |
| 1968 | −0.3509 | −0.33426 | 0 | 0 | 0 | 0.230316 | 9 | 572.7035 | 0.683529 |
| 1969 | −0.35435 | 0 | 0.326983 | 0 | 0.276248 | 0 | 9 | 572.2917 | 0.682264 |
| 1970 | −0.35429 | 0 | 0.335115 | 0 | 0 | 0.278056 | 9 | 571.3971 | 0.68457 |
| 1971 | −0.35918 | 0 | 0 | 0.257296 | 0.231108 | 0 | 9 | 575.7034 | 0.678977 |
| 1972 | −0.36046 | 0 | 0 | 0.27483 | 0 | 0.241127 | 9 | 574.2152 | 0.678977 |
| 1973 | −0.37665 | −0.49016 | 0.415897 | 0.213769 | 0 | 0 | 9 | 563.0963 | 0.700127 |
| 1974 | −0.3692 | −0.49878 | 0.458935 | 0 | 0.257995 | 0 | 9 | 559.774 | 0.705884 |
| 1975 | −0.36789 | −0.4958 | 0.464972 | 0 | 0 | 0.255069 | 9 | 560.9075 | 0.699575 |
| 1976 | −0.36683 | −0.32775 | 0 | 0.247282 | 0.215748 | 0 | 9 | 571.9263 | 0.688817 |
| 1977 | −0.36769 | −0.32552 | 0 | 0.266766 | 0 | 0.223296 | 9 | 572.2165 | 0.6821 |
| 1978 | −0.35346 | −0.32601 | 0 | 0 | 0.190682 | 0.171912 | 9 | 574.5139 | 0.682958 |
| 1979 | −0.36573 | 0 | 0.297911 | 0.217151 | 0.249518 | 0 | 9 | 571.3869 | 0.688123 |
| 1980 | −0.36751 | 0 | 0.304766 | 0.234822 | 0 | 0.26618 | 9 | 571.4659 | 0.684877 |
| 1981 | −0.35702 | 0 | 0.341819 | 0 | 0.21209 | 0.213821 | 9 | 571.435 | 0.68604 |
| 1982 | −0.36116 | 0 | 0 | 0.256551 | 0.172681 | 0.188346 | 9 | 576.3225 | 0.675547 |
| 1983 | −0.37665 | −0.49016 | 0.415897 | 0.213769 | 0 | 0 | 9 | 560.5092 | 0.707272 |
| 1984 | −0.3692 | −0.49878 | 0.458935 | 0 | 0.257995 | 0 | 9 | 556.8198 | 0.71111 |
| 1985 | −0.36789 | −0.4958 | 0.464972 | 0 | 0 | 0.255069 | 9 | 556.2467 | 0.709844 |
| 1986 | −0.36683 | −0.32775 | 0 | 0.247282 | 0.215748 | 0 | 9 | 568.3882 | 0.69441 |
| 1987 | −0.36769 | −0.32552 | 0 | 0.266766 | 0 | 0.223296 | 9 | 567.1146 | 0.692777 |
| 1988 | −0.35346 | −0.32601 | 0 | 0 | 0.190682 | 0.171912 | 9 | 569.6991 | 0.690144 |
| 1989 | −0.36573 | 0 | 0.297911 | 0.217151 | 0.249518 | 0 | 9 | 568.8995 | 0.689919 |
| 1990 | −0.36751 | 0 | 0.304766 | 0.234822 | 0 | 0.26618 | 9 | 567.2532 | 0.692144 |
| 1991 | −0.35702 | 0 | 0.341819 | 0 | 0.21209 | 0.213821 | 9 | 567.3821 | 0.69096 |
| 1992 | −0.36116 | 0 | 0 | 0.256551 | 0.172681 | 0.188346 | 9 | 571.7936 | 0.681692 |
| 1993 | −0.3784 | −0.48038 | 0.429094 | 0.184036 | 0.235336 | 0 | 9 | 557.4568 | 0.708966 |
| 1994 | −0.37944 | −0.47686 | 0.432955 | 0.204998 | 0 | 0.247828 | 9 | 557.8597 | 0.705537 |
| 1995 | −0.37057 | −0.48826 | 0.469023 | 0 | 0.198485 | 0.194003 | 9 | 557.1394 | 0.706231 |
| 1996 | −0.36821 | −0.31823 | 0 | 0.248938 | 0.16128 | 0.17389 | 9 | 570.0901 | 0.687939 |
| 1997 | −0.36844 | 0 | 0.31303 | 0.214129 | 0.186465 | 0.210249 | 9 | 568.2835 | 0.689735 |
| 1998 | −0.37665 | −0.49016 | 0.415897 | 0.213769 | 0 | 0 | 9 | 560.6775 | 0.707496 |
| 1999 | −0.3692 | −0.49878 | 0.458935 | 0 | 0.257995 | 0 | 9 | 557.7901 | 0.708762 |
| 2000 | −0.36789 | −0.4958 | 0.464972 | 0 | 0 | 0.255069 | 9 | 556.4222 | 0.709599 |
| 2001 | −0.36683 | −0.32775 | 0 | 0.247282 | 0.215748 | 0 | 9 | 568.7354 | 0.69488 |
| 2002 | −0.36769 | −0.32552 | 0 | 0.266766 | 0 | 0.223296 | 9 | 566.8818 | 0.694288 |
| 2003 | −0.35346 | −0.32601 | 0 | 0 | 0.190682 | 0.171912 | 9 | 569.8704 | 0.690307 |
| 2004 | −0.36573 | 0 | 0.297911 | 0.217151 | 0.249518 | 0 | 9 | 569.5175 | 0.688184 |
| 2005 | −0.36751 | 0 | 0.304766 | 0.234822 | 0 | 0.26618 | 9 | 567.1498 | 0.690511 |
| 2006 | −0.35702 | 0 | 0.341819 | 0 | 0.21209 | 0.213821 | 9 | 567.8622 | 0.688837 |
| 2007 | −0.36116 | 0 | 0 | 0.256551 | 0.172681 | 0.188346 | 9 | 571.9118 | 0.680732 |
| 2008 | −0.3784 | −0.48038 | 0.429094 | 0.184036 | 0.235336 | 0 | 9 | 558.9969 | 0.706741 |
| 2009 | −0.37944 | −0.47686 | 0.432955 | 0.204998 | 0 | 0.247828 | 9 | 558.5993 | 0.702536 |
| 2010 | −0.37057 | −0.48826 | 0.469023 | 0 | 0.198485 | 0.194003 | 9 | 558.6042 | 0.702658 |
| 2011 | −0.36821 | −0.31823 | 0 | 0.248938 | 0.16128 | 0.17389 | 9 | 570.7881 | 0.686796 |
| 2012 | −0.36844 | 0 | 0.31303 | 0.214129 | 0.186465 | 0.210249 | 9 | 569.3689 | 0.687143 |
| 2013 | −0.3784 | −0.48038 | 0.429094 | 0.184036 | 0.235336 | 0 | 9 | 555.3785 | 0.7136 |
| 2014 | −0.37944 | −0.47686 | 0.432955 | 0.204998 | 0 | 0.247828 | 9 | 553.284 | 0.715621 |
| 2015 | −0.37057 | −0.48826 | 0.469023 | 0 | 0.198485 | 0.194003 | 9 | 553.3896 | 0.712314 |
| 2016 | −0.36821 | −0.31823 | 0 | 0.248938 | 0.16128 | 0.17389 | 9 | 565.194 | 0.696044 |
| 2017 | −0.36844 | 0 | 0.31303 | 0.214129 | 0.186465 | 0.210249 | 9 | 564.5656 | 0.69488 |
| 2018 | −0.38017 | −0.47149 | 0.43946 | 0.184707 | 0.175726 | 0.194494 | 9 | 556.3376 | 0.70521 |
| 2019 | −0.34954 | 0 | 0.311734 | 0 | 0 | 0 | 9 | 508.7334 | 0.781623 |
| 2020 | −0.34838 | −0.35299 | 0 | 0 | 0 | 0 | 9 | 504.929 | 0.784685 |
| 2021 | −0.35731 | 0 | 0 | 0.283735 | 0 | 0 | 9 | 509.0263 | 0.780357 |
| 2022 | −0.34421 | 0 | 0 | 0 | 0.260301 | 0 | 9 | 508.7531 | 0.7805 |
| 2023 | −0.34349 | 0 | 0 | 0 | 0 | 0.25144 | 9 | 508.9283 | 0.78248 |
| 2024 | −0.34954 | 0 | 0.311734 | 0 | 0 | 0 | 9 | 509.2996 | 0.780867 |
| 2025 | −0.34838 | −0.35299 | 0 | 0 | 0 | 0 | 9 | 505.3541 | 0.784685 |
| 2026 | −0.35731 | 0 | 0 | 0.283735 | 0 | 0 | 9 | 509.5209 | 0.778295 |
| 2027 | −0.34421 | 0 | 0 | 0 | 0.260301 | 0 | 9 | 509.7533 | 0.777723 |
| 2028 | −0.34349 | 0 | 0 | 0 | 0 | 0.25144 | 9 | 509.3645 | 0.780153 |
| 2029 | −0.34954 | 0 | 0.311734 | 0 | 0 | 0 | 9 | 505.9431 | 0.785195 |
| 2030 | −0.34838 | −0.35299 | 0 | 0 | 0 | 0 | 9 | 500.4818 | 0.790605 |
| 2031 | −0.35731 | 0 | 0 | 0.283735 | 0 | 0 | 9 | 505.4462 | 0.783827 |
| 2032 | −0.34421 | 0 | 0 | 0 | 0.260301 | 0 | 9 | 505.3633 | 0.784971 |
| 2033 | −0.34349 | 0 | 0 | 0 | 0 | 0.25144 | 9 | 504.0366 | 0.787788 |
| 2034 | −0.3635 | 0 | 0.280897 | 0.247949 | 0 | 0 | 9 | 507.5486 | 0.780826 |
| 2035 | −0.36484 | −0.51223 | 0.448932 | 0 | 0 | 0 | 9 | 497.792 | 0.794525 |
| 2036 | −0.36535 | −0.34122 | 0 | 0.272545 | 0 | 0 | 9 | 503.1311 | 0.786114 |
| 2037 | −0.35247 | −0.33749 | 0 | 0 | 0.244058 | 0 | 9 | 503.5228 | 0.785216 |
| 2038 | −0.3509 | −0.33426 | 0 | 0 | 0 | 0.230316 | 9 | 503.3484 | 0.787135 |
| 2039 | −0.35435 | 0 | 0.326983 | 0 | 0.276248 | 0 | 9 | 506.8562 | 0.782929 |
| 2040 | −0.35429 | 0 | 0.335115 | 0 | 0 | 0.278056 | 9 | 506.281 | 0.784358 |
| 2041 | −0.35918 | 0 | 0 | 0.257296 | 0.231108 | 0 | 9 | 507.956 | 0.780091 |
| 2042 | −0.36046 | 0 | 0 | 0.27483 | 0 | 0.241127 | 9 | 507.2087 | 0.781255 |
| 2043 | −0.34604 | 0 | 0 | 0 | 0.20186 | 0.189915 | 9 | 508.7114 | 0.780234 |
| 2044 | −0.34954 | 0 | 0.311734 | 0 | 0 | 0 | 9 | 508.0311 | 0.782296 |
| 2045 | −0.34838 | −0.35299 | 0 | 0 | 0 | 0 | 9 | 504.2402 | 0.785644 |
| 2046 | −0.35731 | 0 | 0 | 0.283735 | 0 | 0 | 9 | 508.2563 | 0.779928 |
| 2047 | −0.34421 | 0 | 0 | 0 | 0.260301 | 0 | 9 | 507.9741 | 0.781418 |
| 2048 | −0.34349 | 0 | 0 | 0 | 0 | 0.25144 | 9 | 508.0824 | 0.782255 |
| 2049 | −0.34954 | 0 | 0.311734 | 0 | 0 | 0 | 9 | 505.3005 | 0.78693 |
| 2050 | −0.34838 | −0.35299 | 0 | 0 | 0 | 0 | 9 | 500.0772 | 0.791483 |
| 2051 | −0.35731 | 0 | 0 | 0.283735 | 0 | 0 | 9 | 504.8098 | 0.785767 |
| 2052 | −0.34421 | 0 | 0 | 0 | 0.260301 | 0 | 9 | 504.3951 | 0.786624 |
| 2053 | −0.34349 | 0 | 0 | 0 | 0 | 0.25144 | 9 | 503.4525 | 0.789135 |
| 2054 | −0.3635 | 0 | 0.280897 | 0.247949 | 0 | 0 | 9 | 506.1 | 0.784562 |
| 2055 | −0.36484 | −0.51223 | 0.448932 | 0 | 0 | 0 | 9 | 496.3342 | 0.794586 |
| 2056 | −0.36535 | −0.34122 | 0 | 0.272545 | 0 | 0 | 9 | 501.7736 | 0.787359 |
| 2057 | −0.35247 | −0.33749 | 0 | 0 | 0.244058 | 0 | 9 | 501.5967 | 0.787135 |
| 2058 | −0.3509 | −0.33426 | 0 | 0 | 0 | 0.230316 | 9 | 501.9978 | 0.788747 |
| 2059 | −0.35435 | 0 | 0.326983 | 0 | 0.276248 | 0 | 9 | 504.714 | 0.785787 |
| 2060 | −0.35429 | 0 | 0.335115 | 0 | 0 | 0.278056 | 9 | 504.7732 | 0.786338 |
| 2061 | −0.35918 | 0 | 0 | 0.257296 | 0.231108 | 0 | 9 | 506.0209 | 0.783929 |
| 2062 | −0.36046 | 0 | 0 | 0.27483 | 0 | 0.241127 | 9 | 505.7832 | 0.784113 |
| 2063 | −0.34604 | 0 | 0 | 0 | 0.20186 | 0.189915 | 9 | 506.8776 | 0.782909 |
| 2064 | −0.34954 | 0 | 0.311734 | 0 | 0 | 0 | 9 | 505.2767 | 0.785828 |
| 2065 | −0.34838 | −0.35299 | 0 | 0 | 0 | 0 | 9 | 499.9456 | 0.792136 |
| 2066 | −0.35731 | 0 | 0 | 0.283735 | 0 | 0 | 9 | 504.7212 | 0.784664 |
| 2067 | −0.34421 | 0 | 0 | 0 | 0.260301 | 0 | 9 | 504.6353 | 0.785011 |
| 2068 | −0.34349 | 0 | 0 | 0 | 0 | 0.25144 | 9 | 503.237 | 0.789135 |
| 2069 | −0.3635 | 0 | 0.280897 | 0.247949 | 0 | 0 | 9 | 506.8646 | 0.782378 |
| 2070 | −0.36484 | −0.51223 | 0.448932 | 0 | 0 | 0 | 9 | 497.2334 | 0.794566 |
| 2071 | −0.36535 | −0.34122 | 0 | 0.272545 | 0 | 0 | 9 | 502.4459 | 0.78742 |
| 2072 | −0.35247 | −0.33749 | 0 | 0 | 0.244058 | 0 | 9 | 502.8369 | 0.786338 |
| 2073 | −0.3509 | −0.33426 | 0 | 0 | 0 | 0.230316 | 9 | 502.616 | 0.788298 |
| 2074 | −0.35435 | 0 | 0.326983 | 0 | 0.276248 | 0 | 9 | 506.1859 | 0.783582 |
| 2075 | −0.35429 | 0 | 0.335115 | 0 | 0 | 0.278056 | 9 | 505.5928 | 0.78495 |
| 2076 | −0.35918 | 0 | 0 | 0.257296 | 0.231108 | 0 | 9 | 507.2571 | 0.780336 |
| 2077 | −0.36046 | 0 | 0 | 0.27483 | 0 | 0.241127 | 9 | 506.5158 | 0.78201 |
| 2078 | −0.34604 | 0 | 0 | 0 | 0.20186 | 0.189915 | 9 | 507.9571 | 0.780561 |
| 2079 | −0.3635 | 0 | 0.280897 | 0.247949 | 0 | 0 | 9 | 503.2305 | 0.787604 |
| 2080 | −0.36484 | −0.51223 | 0.448932 | 0 | 0 | 0 | 9 | 492.7999 | 0.801609 |
| 2081 | −0.36535 | −0.34122 | 0 | 0.272545 | 0 | 0 | 9 | 497.2065 | 0.79381 |
| 2082 | −0.35247 | −0.33749 | 0 | 0 | 0.244058 | 0 | 9 | 497.2735 | 0.793177 |
| 2083 | −0.3509 | −0.33426 | 0 | 0 | 0 | 0.230316 | 9 | 495.8751 | 0.796219 |
| 2084 | −0.35435 | 0 | 0.326983 | 0 | 0.276248 | 0 | 9 | 502.2019 | 0.788686 |
| 2085 | −0.35429 | 0 | 0.335115 | 0 | 0 | 0.278056 | 9 | 500.5574 | 0.790932 |
| 2086 | −0.35918 | 0 | 0 | 0.257296 | 0.231108 | 0 | 9 | 502.5242 | 0.787461 |
| 2087 | −0.36046 | 0 | 0 | 0.27483 | 0 | 0.241127 | 9 | 500.6791 | 0.790503 |
| 2088 | −0.34604 | 0 | 0 | 0 | 0.20186 | 0.189915 | 9 | 502.331 | 0.788666 |
| 2089 | −0.37665 | −0.49016 | 0.415897 | 0.213769 | 0 | 0 | 9 | 495.99 | 0.796423 |
| 2090 | −0.3692 | −0.49878 | 0.458935 | 0 | 0.257995 | 0 | 9 | 495.0794 | 0.795811 |
| 2091 | −0.36789 | −0.4958 | 0.464972 | 0 | 0 | 0.255069 | 9 | 494.7279 | 0.797546 |
| 2092 | −0.36683 | −0.32775 | 0 | 0.247282 | 0.215748 | 0 | 9 | 501.1723 | 0.787482 |
| 2093 | −0.36769 | −0.32552 | 0 | 0.266766 | 0 | 0.223296 | 9 | 500.6323 | 0.79034 |
| 2094 | −0.35346 | −0.32601 | 0 | 0 | 0.190682 | 0.171912 | 9 | 502.1043 | 0.786828 |
| 2095 | −0.36573 | 0 | 0.297911 | 0.217151 | 0.249518 | 0 | 9 | 505.0225 | 0.784134 |
| 2096 | −0.36751 | 0 | 0.304766 | 0.234822 | 0 | 0.26618 | 9 | 504.1861 | 0.785542 |
| 2097 | −0.35702 | 0 | 0.341819 | 0 | 0.21209 | 0.213821 | 9 | 504.7955 | 0.784399 |
| 2098 | −0.36116 | 0 | 0 | 0.256551 | 0.172681 | 0.188346 | 9 | 506.3034 | 0.781602 |
| 2099 | −0.34954 | 0 | 0.311734 | 0 | 0 | 0 | 9 | 511.2852 | 0.77803 |
| 2100 | −0.34838 | −0.35299 | 0 | 0 | 0 | 0 | 9 | 507.3569 | 0.781153 |
| 2101 | −0.35731 | 0 | 0 | 0.283735 | 0 | 0 | 9 | 511.0197 | 0.777213 |
| 2102 | −0.34421 | 0 | 0 | 0 | 0.260301 | 0 | 9 | 511.0533 | 0.77807 |
| 2103 | −0.34349 | 0 | 0 | 0 | 0 | 0.25144 | 9 | 510.9781 | 0.778193 |
| 2104 | −0.34954 | 0 | 0.311734 | 0 | 0 | 0 | 9 | 508.6254 | 0.784032 |
| 2105 | −0.34838 | −0.35299 | 0 | 0 | 0 | 0 | 9 | 503.4019 | 0.787767 |
| 2106 | −0.35731 | 0 | 0 | 0.283735 | 0 | 0 | 9 | 507.7103 | 0.782786 |
| 2107 | −0.34421 | 0 | 0 | 0 | 0.260301 | 0 | 9 | 507.631 | 0.783807 |
| 2108 | −0.34349 | 0 | 0 | 0 | 0 | 0.25144 | 9 | 506.6556 | 0.783725 |
| 2109 | −0.3635 | 0 | 0.280897 | 0.247949 | 0 | 0 | 9 | 508.9782 | 0.780418 |
| 2110 | −0.36484 | −0.51223 | 0.448932 | 0 | 0 | 0 | 9 | 500.0736 | 0.79085 |
| 2111 | −0.36535 | −0.34122 | 0 | 0.272545 | 0 | 0 | 9 | 504.5353 | 0.784113 |
| 2112 | −0.35247 | −0.33749 | 0 | 0 | 0.244058 | 0 | 9 | 504.6982 | 0.784236 |
| 2113 | −0.3509 | −0.33426 | 0 | 0 | 0 | 0.230316 | 9 | 504.873 | 0.784521 |
| 2114 | −0.35435 | 0 | 0.326983 | 0 | 0.276248 | 0 | 9 | 508.0635 | 0.78197 |
| 2115 | −0.35429 | 0 | 0.335115 | 0 | 0 | 0.278056 | 9 | 507.8593 | 0.782807 |
| 2116 | −0.35918 | 0 | 0 | 0.257296 | 0.231108 | 0 | 9 | 508.6933 | 0.780296 |
| 2117 | −0.36046 | 0 | 0 | 0.27483 | 0 | 0.241127 | 9 | 508.2294 | 0.780663 |
| 2118 | −0.34604 | 0 | 0 | 0 | 0.20186 | 0.189915 | 9 | 509.6421 | 0.779949 |
| 2119 | −0.34954 | 0 | 0.311734 | 0 | 0 | 0 | 9 | 509.1351 | 0.782113 |
| 2120 | −0.34838 | −0.35299 | 0 | 0 | 0 | 0 | 9 | 503.7534 | 0.786665 |
| 2121 | −0.35731 | 0 | 0 | 0.283735 | 0 | 0 | 9 | 508.1363 | 0.78099 |
| 2122 | −0.34421 | 0 | 0 | 0 | 0.260301 | 0 | 9 | 508.4577 | 0.78201 |
| 2123 | −0.34349 | 0 | 0 | 0 | 0 | 0.25144 | 9 | 506.9737 | 0.783684 |
| 2124 | −0.3635 | 0 | 0.280897 | 0.247949 | 0 | 0 | 9 | 510.2238 | 0.778152 |
| 2125 | −0.36484 | −0.51223 | 0.448932 | 0 | 0 | 0 | 9 | 501.4732 | 0.790319 |
| 2126 | −0.36535 | −0.34122 | 0 | 0.272545 | 0 | 0 | 9 | 505.6734 | 0.781643 |
| 2127 | −0.35247 | −0.33749 | 0 | 0 | 0.244058 | 0 | 9 | 506.4348 | 0.781255 |
| 2128 | −0.3509 | −0.33426 | 0 | 0 | 0 | 0.230316 | 9 | 505.9571 | 0.78199 |
| 2129 | −0.35435 | 0 | 0.326983 | 0 | 0.276248 | 0 | 9 | 510.0255 | 0.778826 |
| 2130 | −0.35429 | 0 | 0.335115 | 0 | 0 | 0.278056 | 9 | 509.1514 | 0.779193 |
| 2131 | −0.35918 | 0 | 0 | 0.257296 | 0.231108 | 0 | 9 | 510.4012 | 0.776294 |
| 2132 | −0.36046 | 0 | 0 | 0.27483 | 0 | 0.241127 | 9 | 509.4052 | 0.778275 |
| 2133 | −0.34604 | 0 | 0 | 0 | 0.20186 | 0.189915 | 9 | 511.189 | 0.776131 |
| 2134 | −0.3635 | 0 | 0.280897 | 0.247949 | 0 | 0 | 9 | 506.7438 | 0.783174 |
| 2135 | −0.36484 | −0.51223 | 0.448932 | 0 | 0 | 0 | 9 | 497.3229 | 0.796382 |
| 2136 | −0.36535 | −0.34122 | 0 | 0.272545 | 0 | 0 | 9 | 500.7446 | 0.788849 |
| 2137 | −0.35247 | −0.33749 | 0 | 0 | 0.244058 | 0 | 9 | 501.2553 | 0.7884 |
| 2138 | −0.3509 | −0.33426 | 0 | 0 | 0 | 0.230316 | 9 | 499.7559 | 0.790564 |
| 2139 | −0.35435 | 0 | 0.326983 | 0 | 0.276248 | 0 | 9 | 506.2791 | 0.783337 |
| 2140 | −0.35429 | 0 | 0.335115 | 0 | 0 | 0.278056 | 9 | 504.5156 | 0.785542 |
| 2141 | −0.35918 | 0 | 0 | 0.257296 | 0.231108 | 0 | 9 | 505.9735 | 0.783378 |
| 2142 | −0.36046 | 0 | 0 | 0.27483 | 0 | 0.241127 | 9 | 504.011 | 0.785052 |
| 2143 | −0.34604 | 0 | 0 | 0 | 0.20186 | 0.189915 | 9 | 506.0132 | 0.784093 |
| 2144 | −0.37665 | −0.49016 | 0.415897 | 0.213769 | 0 | 0 | 9 | 499.7518 | 0.790911 |
| 2145 | −0.3692 | −0.49878 | 0.458935 | 0 | 0.257995 | 0 | 9 | 499.3526 | 0.79083 |
| 2146 | −0.36789 | −0.4958 | 0.464972 | 0 | 0 | 0.255069 | 9 | 498.664 | 0.792994 |
| 2147 | −0.36683 | −0.32775 | 0 | 0.247282 | 0.215748 | 0 | 9 | 504.2295 | 0.784154 |
| 2148 | −0.36769 | −0.32552 | 0 | 0.266766 | 0 | 0.223296 | 9 | 503.3886 | 0.785175 |
| 2149 | −0.35346 | −0.32601 | 0 | 0 | 0.190682 | 0.171912 | 9 | 505.2488 | 0.783378 |
| 2150 | −0.36573 | 0 | 0.297911 | 0.217151 | 0.249518 | 0 | 9 | 508.2849 | 0.780296 |
| 2151 | −0.36751 | 0 | 0.304766 | 0.234822 | 0 | 0.26618 | 9 | 507.1233 | 0.781031 |
| 2152 | −0.35702 | 0 | 0.341819 | 0 | 0.21209 | 0.213821 | 9 | 508.1793 | 0.780541 |
| 2153 | −0.36116 | 0 | 0 | 0.256551 | 0.172681 | 0.188346 | 9 | 508.9045 | 0.778601 |
| 2154 | −0.34954 | 0 | 0.311734 | 0 | 0 | 0 | 9 | 508.4991 | 0.784011 |
| 2155 | −0.34838 | −0.35299 | 0 | 0 | 0 | 0 | 9 | 503.4144 | 0.786073 |
| 2156 | −0.35731 | 0 | 0 | 0.283735 | 0 | 0 | 9 | 507.487 | 0.782419 |
| 2157 | −0.34421 | 0 | 0 | 0 | 0.260301 | 0 | 9 | 507.4717 | 0.783889 |
| 2158 | −0.34349 | 0 | 0 | 0 | 0 | 0.25144 | 9 | 506.3705 | 0.784624 |
| 2159 | −0.3635 | 0 | 0.280897 | 0.247949 | 0 | 0 | 9 | 508.6244 | 0.78052 |
| 2160 | −0.36484 | −0.51223 | 0.448932 | 0 | 0 | 0 | 9 | 499.924 | 0.79087 |
| 2161 | −0.36535 | −0.34122 | 0 | 0.272545 | 0 | 0 | 9 | 504.1891 | 0.783909 |
| 2162 | −0.35247 | −0.33749 | 0 | 0 | 0.244058 | 0 | 9 | 504.3643 | 0.783521 |
| 2163 | −0.3509 | −0.33426 | 0 | 0 | 0 | 0.230316 | 9 | 504.4292 | 0.784338 |
| 2164 | −0.35435 | 0 | 0.326983 | 0 | 0.276248 | 0 | 9 | 507.7359 | 0.782562 |
| 2165 | −0.35429 | 0 | 0.335115 | 0 | 0 | 0.278056 | 9 | 507.4386 | 0.7825 |
| 2166 | −0.35918 | 0 | 0 | 0.257296 | 0.231108 | 0 | 9 | 508.2742 | 0.781296 |
| 2167 | −0.36046 | 0 | 0 | 0.27483 | 0 | 0.241127 | 9 | 507.7436 | 0.781031 |
| 2168 | −0.34604 | 0 | 0 | 0 | 0.20186 | 0.189915 | 9 | 509.1415 | 0.780806 |
| 2169 | −0.3635 | 0 | 0.280897 | 0.247949 | 0 | 0 | 9 | 505.8917 | 0.783644 |
| 2170 | −0.36484 | −0.51223 | 0.448932 | 0 | 0 | 0 | 9 | 496.6651 | 0.796505 |
| 2171 | −0.36535 | −0.34122 | 0 | 0.272545 | 0 | 0 | 9 | 500.1152 | 0.788829 |
| 2172 | −0.35247 | −0.33749 | 0 | 0 | 0.244058 | 0 | 9 | 500.2695 | 0.789891 |
| 2173 | −0.3509 | −0.33426 | 0 | 0 | 0 | 0.230316 | 9 | 499.1912 | 0.791667 |
| 2174 | −0.35435 | 0 | 0.326983 | 0 | 0.276248 | 0 | 9 | 504.9528 | 0.785787 |
| 2175 | −0.35429 | 0 | 0.335115 | 0 | 0 | 0.278056 | 9 | 503.6549 | 0.787584 |
| 2176 | −0.35918 | 0 | 0 | 0.257296 | 0.231108 | 0 | 9 | 504.7761 | 0.785685 |
| 2177 | −0.36046 | 0 | 0 | 0.27483 | 0 | 0.241127 | 9 | 503.1854 | 0.786522 |
| 2178 | −0.34604 | 0 | 0 | 0 | 0.20186 | 0.189915 | 9 | 504.9385 | 0.785767 |
| 2179 | −0.37665 | −0.49016 | 0.415897 | 0.213769 | 0 | 0 | 9 | 497.9664 | 0.792524 |
| 2180 | −0.3692 | −0.49878 | 0.458935 | 0 | 0.257995 | 0 | 9 | 496.7698 | 0.794178 |
| 2181 | −0.36789 | −0.4958 | 0.464972 | 0 | 0 | 0.255069 | 9 | 496.7532 | 0.794627 |
| 2182 | −0.36683 | −0.32775 | 0 | 0.247282 | 0.215748 | 0 | 9 | 501.9761 | 0.786563 |
| 2183 | −0.36769 | −0.32552 | 0 | 0.266766 | 0 | 0.223296 | 9 | 501.6367 | 0.787012 |
| 2184 | −0.35346 | −0.32601 | 0 | 0 | 0.190682 | 0.171912 | 9 | 503.0858 | 0.786624 |
| 2185 | −0.36573 | 0 | 0.297911 | 0.217151 | 0.249518 | 0 | 9 | 505.8548 | 0.784603 |
| 2186 | −0.36751 | 0 | 0.304766 | 0.234822 | 0 | 0.26618 | 9 | 505.2604 | 0.784256 |
| 2187 | −0.35702 | 0 | 0.341819 | 0 | 0.21209 | 0.213821 | 9 | 505.7994 | 0.784276 |
| 2188 | −0.36116 | 0 | 0 | 0.256551 | 0.172681 | 0.188346 | 9 | 506.727 | 0.783133 |
| 2189 | −0.3635 | 0 | 0.280897 | 0.247949 | 0 | 0 | 9 | 506.4675 | 0.783684 |
| 2190 | −0.36484 | −0.51223 | 0.448932 | 0 | 0 | 0 | 9 | 497.3037 | 0.795954 |
| 2191 | −0.36535 | −0.34122 | 0 | 0.272545 | 0 | 0 | 9 | 500.5679 | 0.788543 |
| 2192 | −0.35247 | −0.33749 | 0 | 0 | 0.244058 | 0 | 9 | 501.1156 | 0.788666 |
| 2193 | −0.3509 | −0.33426 | 0 | 0 | 0 | 0.230316 | 9 | 499.5222 | 0.791115 |
| 2194 | −0.35435 | 0 | 0.326983 | 0 | 0.276248 | 0 | 9 | 506.0211 | 0.783807 |
| 2195 | −0.35429 | 0 | 0.335115 | 0 | 0 | 0.278056 | 9 | 504.1812 | 0.785114 |
| 2196 | −0.35918 | 0 | 0 | 0.257296 | 0.231108 | 0 | 9 | 505.6536 | 0.783174 |
| 2197 | −0.36046 | 0 | 0 | 0.27483 | 0 | 0.241127 | 9 | 503.6194 | 0.785665 |
| 2198 | −0.34604 | 0 | 0 | 0 | 0.20186 | 0.189915 | 9 | 505.6439 | 0.784338 |
| 2199 | −0.37665 | −0.49016 | 0.415897 | 0.213769 | 0 | 0 | 9 | 499.483 | 0.790932 |
| 2200 | −0.3692 | −0.49878 | 0.458935 | 0 | 0.257995 | 0 | 9 | 499.0986 | 0.791156 |
| 2201 | −0.36789 | −0.4958 | 0.464972 | 0 | 0 | 0.255069 | 9 | 498.3611 | 0.792361 |
| 2202 | −0.36683 | −0.32775 | 0 | 0.247282 | 0.215748 | 0 | 9 | 503.8636 | 0.783807 |
| 2203 | −0.36769 | −0.32552 | 0 | 0.266766 | 0 | 0.223296 | 9 | 502.9976 | 0.785338 |
| 2204 | −0.35346 | −0.32601 | 0 | 0 | 0.190682 | 0.171912 | 9 | 504.8236 | 0.783766 |
| 2205 | −0.36573 | 0 | 0.297911 | 0.217151 | 0.249518 | 0 | 9 | 507.9448 | 0.781051 |
| 2206 | −0.36751 | 0 | 0.304766 | 0.234822 | 0 | 0.26618 | 9 | 506.7737 | 0.780928 |
| 2207 | −0.35702 | 0 | 0.341819 | 0 | 0.21209 | 0.213821 | 9 | 507.8122 | 0.780826 |
| 2208 | −0.36116 | 0 | 0 | 0.256551 | 0.172681 | 0.188346 | 9 | 508.5043 | 0.77852 |
| 2209 | −0.37665 | −0.49016 | 0.415897 | 0.213769 | 0 | 0 | 9 | 495.1907 | 0.796668 |
| 2210 | −0.3692 | −0.49878 | 0.458935 | 0 | 0.257995 | 0 | 9 | 494.4852 | 0.797322 |
| 2211 | −0.36789 | −0.4958 | 0.464972 | 0 | 0 | 0.255069 | 9 | 492.6282 | 0.799404 |
| 2212 | −0.36683 | −0.32775 | 0 | 0.247282 | 0.215748 | 0 | 9 | 498.4772 | 0.791258 |
| 2213 | −0.36769 | −0.32552 | 0 | 0.266766 | 0 | 0.223296 | 9 | 496.3922 | 0.794035 |
| 2214 | −0.35346 | −0.32601 | 0 | 0 | 0.190682 | 0.171912 | 9 | 498.5927 | 0.791891 |
| 2215 | −0.36573 | 0 | 0.297911 | 0.217151 | 0.249518 | 0 | 9 | 504.0126 | 0.78591 |
| 2216 | −0.36751 | 0 | 0.304766 | 0.234822 | 0 | 0.26618 | 9 | 501.8007 | 0.788135 |
| 2217 | −0.35702 | 0 | 0.341819 | 0 | 0.21209 | 0.213821 | 9 | 503.0312 | 0.787196 |
| 2218 | −0.36116 | 0 | 0 | 0.256551 | 0.172681 | 0.188346 | 9 | 502.9348 | 0.785808 |
| 2219 | −0.3784 | −0.48038 | 0.429094 | 0.184036 | 0.235336 | 0 | 9 | 497.7701 | 0.792892 |
| 2220 | −0.37944 | −0.47686 | 0.432955 | 0.204998 | 0 | 0.247828 | 9 | 496.8025 | 0.794463 |
| 2221 | −0.37057 | −0.48826 | 0.469023 | 0 | 0.198485 | 0.194003 | 9 | 497.5155 | 0.792912 |
| 2222 | −0.36821 | −0.31823 | 0 | 0.248938 | 0.16128 | 0.17389 | 9 | 502.6298 | 0.786359 |
| 2223 | −0.36844 | 0 | 0.31303 | 0.214129 | 0.186465 | 0.210249 | 9 | 506.1635 | 0.782133 |
| 2224 | −0.34954 | 0 | 0.311734 | 0 | 0 | 0 | 9 | 547.4342 | 0.729891 |
| 2225 | −0.34838 | −0.35299 | 0 | 0 | 0 | 0 | 9 | 555.8457 | 0.713253 |
| 2226 | −0.35731 | 0 | 0 | 0.283735 | 0 | 0 | 9 | 553.8665 | 0.719623 |
| 2227 | −0.34421 | 0 | 0 | 0 | 0.260301 | 0 | 9 | 554.0883 | 0.72242 |
| 2228 | −0.34349 | 0 | 0 | 0 | 0 | 0.25144 | 9 | 554.2242 | 0.718827 |
| 2229 | −0.34954 | 0 | 0.311734 | 0 | 0 | 0 | 9 | 545.99 | 0.732709 |
| 2230 | −0.34838 | −0.35299 | 0 | 0 | 0 | 0 | 9 | 553.4477 | 0.71703 |
| 2231 | −0.35731 | 0 | 0 | 0.283735 | 0 | 0 | 9 | 551.8191 | 0.722705 |
| 2232 | −0.34421 | 0 | 0 | 0 | 0.260301 | 0 | 9 | 551.8128 | 0.722562 |
| 2233 | −0.34349 | 0 | 0 | 0 | 0 | 0.25144 | 9 | 550.8856 | 0.725359 |
| 2234 | −0.3635 | 0 | 0.280897 | 0.247949 | 0 | 0 | 9 | 544.7375 | 0.734199 |
| 2235 | −0.36484 | −0.51223 | 0.448932 | 0 | 0 | 0 | 9 | 539.0019 | 0.739119 |
| 2236 | −0.36535 | −0.34122 | 0 | 0.272545 | 0 | 0 | 9 | 551.9663 | 0.720194 |
| 2237 | −0.35247 | −0.33749 | 0 | 0 | 0.244058 | 0 | 9 | 552.0783 | 0.723359 |
| 2238 | −0.3509 | −0.33426 | 0 | 0 | 0 | 0.230316 | 9 | 552.5351 | 0.718745 |
| 2239 | −0.35435 | 0 | 0.326983 | 0 | 0.276248 | 0 | 9 | 542.3738 | 0.736996 |
| 2240 | −0.35429 | 0 | 0.335115 | 0 | 0 | 0.278056 | 9 | 542.2605 | 0.736057 |
| 2241 | −0.35918 | 0 | 0 | 0.257296 | 0.231108 | 0 | 9 | 550.7474 | 0.724094 |
| 2242 | −0.36046 | 0 | 0 | 0.27483 | 0 | 0.241127 | 9 | 550.29 | 0.723706 |
| 2243 | −0.34604 | 0 | 0 | 0 | 0.20186 | 0.189915 | 9 | 552.3436 | 0.72144 |
| 2244 | −0.34954 | 0 | 0.311734 | 0 | 0 | 0 | 9 | 545.9741 | 0.732464 |
| 2245 | −0.34838 | −0.35299 | 0 | 0 | 0 | 0 | 9 | 553.1675 | 0.718806 |
| 2246 | −0.35731 | 0 | 0 | 0.283735 | 0 | 0 | 9 | 551.5242 | 0.723971 |
| 2247 | −0.34421 | 0 | 0 | 0 | 0.260301 | 0 | 9 | 552.0689 | 0.725053 |
| 2248 | −0.34349 | 0 | 0 | 0 | 0 | 0.25144 | 9 | 550.5098 | 0.725359 |
| 2249 | −0.3635 | 0 | 0.280897 | 0.247949 | 0 | 0 | 9 | 545.1317 | 0.731749 |
| 2250 | −0.36484 | −0.51223 | 0.448932 | 0 | 0 | 0 | 9 | 539.5657 | 0.738119 |
| 2251 | −0.36535 | −0.34122 | 0 | 0.272545 | 0 | 0 | 9 | 552.0713 | 0.720133 |
| 2252 | −0.35247 | −0.33749 | 0 | 0 | 0.244058 | 0 | 9 | 552.7973 | 0.722297 |
| 2253 | −0.3509 | −0.33426 | 0 | 0 | 0 | 0.230316 | 9 | 552.6314 | 0.719929 |
| 2254 | −0.35435 | 0 | 0.326983 | 0 | 0.276248 | 0 | 9 | 543.7931 | 0.734076 |
| 2255 | −0.35429 | 0 | 0.335115 | 0 | 0 | 0.278056 | 9 | 542.7823 | 0.73526 |
| 2256 | −0.35918 | 0 | 0 | 0.257296 | 0.231108 | 0 | 9 | 551.5279 | 0.721133 |
| 2257 | −0.36046 | 0 | 0 | 0.27483 | 0 | 0.241127 | 9 | 550.4628 | 0.722767 |
| 2258 | −0.34604 | 0 | 0 | 0 | 0.20186 | 0.189915 | 9 | 553.0148 | 0.721011 |
| 2259 | −0.3635 | 0 | 0.280897 | 0.247949 | 0 | 0 | 9 | 543.1336 | 0.737384 |
| 2260 | −0.36484 | −0.51223 | 0.448932 | 0 | 0 | 0 | 9 | 537.1506 | 0.741324 |
| 2261 | −0.36535 | −0.34122 | 0 | 0.272545 | 0 | 0 | 9 | 549.1335 | 0.723644 |
| 2262 | −0.35247 | −0.33749 | 0 | 0 | 0.244058 | 0 | 9 | 549.5272 | 0.724563 |
| 2263 | −0.3509 | −0.33426 | 0 | 0 | 0 | 0.230316 | 9 | 548.3798 | 0.725625 |
| 2264 | −0.35435 | 0 | 0.326983 | 0 | 0.276248 | 0 | 9 | 541.316 | 0.736342 |
| 2265 | −0.35429 | 0 | 0.335115 | 0 | 0 | 0.278056 | 9 | 539.0983 | 0.741548 |
| 2266 | −0.35918 | 0 | 0 | 0.257296 | 0.231108 | 0 | 9 | 548.5774 | 0.726196 |
| 2267 | −0.36046 | 0 | 0 | 0.27483 | 0 | 0.241127 | 9 | 546.3871 | 0.730769 |
| 2268 | −0.34604 | 0 | 0 | 0 | 0.20186 | 0.189915 | 9 | 549.1132 | 0.727135 |
| 2269 | −0.37665 | −0.49016 | 0.415897 | 0.213769 | 0 | 0 | 9 | 537.6675 | 0.740099 |
| 2270 | −0.3692 | −0.49878 | 0.458935 | 0 | 0.257995 | 0 | 9 | 535.7177 | 0.742385 |
| 2271 | −0.36789 | −0.4958 | 0.464972 | 0 | 0 | 0.255069 | 9 | 535.2251 | 0.744304 |
| 2272 | −0.36683 | −0.32775 | 0 | 0.247282 | 0.215748 | 0 | 9 | 549.8023 | 0.722603 |
| 2273 | −0.36769 | −0.32552 | 0 | 0.266766 | 0 | 0.223296 | 9 | 549.0344 | 0.72246 |
| 2274 | −0.35346 | −0.32601 | 0 | 0 | 0.190682 | 0.171912 | 9 | 551.341 | 0.720501 |
| 2275 | −0.36573 | 0 | 0.297911 | 0.217151 | 0.249518 | 0 | 9 | 541.9001 | 0.735465 |
| 2276 | −0.36751 | 0 | 0.304766 | 0.234822 | 0 | 0.26618 | 9 | 540.4978 | 0.737914 |
| 2277 | −0.35702 | 0 | 0.341819 | 0 | 0.21209 | 0.213821 | 9 | 541.065 | 0.736077 |
| 2278 | −0.36116 | 0 | 0 | 0.256551 | 0.172681 | 0.188346 | 9 | 549.7738 | 0.722256 |
| 2279 | −0.34954 | 0 | 0.311734 | 0 | 0 | 0 | 9 | 545.6786 | 0.733995 |
| 2280 | −0.34838 | −0.35299 | 0 | 0 | 0 | 0 | 9 | 553.1245 | 0.719337 |
| 2281 | −0.35731 | 0 | 0 | 0.283735 | 0 | 0 | 9 | 551.2239 | 0.723971 |
| 2282 | −0.34421 | 0 | 0 | 0 | 0.260301 | 0 | 9 | 551.3389 | 0.7254 |
| 2283 | −0.34349 | 0 | 0 | 0 | 0 | 0.25144 | 9 | 550.2225 | 0.72687 |
| 2284 | −0.3635 | 0 | 0.280897 | 0.247949 | 0 | 0 | 9 | 544.2413 | 0.734648 |
| 2285 | −0.36484 | −0.51223 | 0.448932 | 0 | 0 | 0 | 9 | 538.7969 | 0.739854 |
| 2286 | −0.36535 | −0.34122 | 0 | 0.272545 | 0 | 0 | 9 | 551.3753 | 0.721072 |
| 2287 | −0.35247 | −0.33749 | 0 | 0 | 0.244058 | 0 | 9 | 551.5514 | 0.725645 |
| 2288 | −0.3509 | −0.33426 | 0 | 0 | 0 | 0.230316 | 9 | 551.8894 | 0.720664 |
| 2289 | −0.35435 | 0 | 0.326983 | 0 | 0.276248 | 0 | 9 | 541.9978 | 0.737486 |
| 2290 | −0.35429 | 0 | 0.335115 | 0 | 0 | 0.278056 | 9 | 541.732 | 0.737996 |
| 2291 | −0.35918 | 0 | 0 | 0.257296 | 0.231108 | 0 | 9 | 550.1028 | 0.724747 |
| 2292 | −0.36046 | 0 | 0 | 0.27483 | 0 | 0.241127 | 9 | 549.5539 | 0.724788 |
| 2293 | −0.34604 | 0 | 0 | 0 | 0.20186 | 0.189915 | 9 | 551.6568 | 0.723624 |
| 2294 | −0.3635 | 0 | 0.280897 | 0.247949 | 0 | 0 | 9 | 542.6343 | 0.736445 |
| 2295 | −0.36484 | −0.51223 | 0.448932 | 0 | 0 | 0 | 9 | 536.8716 | 0.742365 |
| 2296 | −0.36535 | −0.34122 | 0 | 0.272545 | 0 | 0 | 9 | 548.8392 | 0.725911 |
| 2297 | −0.35247 | −0.33749 | 0 | 0 | 0.244058 | 0 | 9 | 548.8562 | 0.726033 |
| 2298 | −0.3509 | −0.33426 | 0 | 0 | 0 | 0.230316 | 9 | 548.1174 | 0.72734 |
| 2299 | −0.35435 | 0 | 0.326983 | 0 | 0.276248 | 0 | 9 | 540.1351 | 0.740017 |
| 2300 | −0.35429 | 0 | 0.335115 | 0 | 0 | 0.278056 | 9 | 538.5683 | 0.743161 |
| 2301 | −0.35918 | 0 | 0 | 0.257296 | 0.231108 | 0 | 9 | 547.6754 | 0.72836 |
| 2302 | −0.36046 | 0 | 0 | 0.27483 | 0 | 0.241127 | 9 | 545.9112 | 0.732586 |
| 2303 | −0.34604 | 0 | 0 | 0 | 0.20186 | 0.189915 | 9 | 548.321 | 0.727911 |
| 2304 | −0.37665 | −0.49016 | 0.415897 | 0.213769 | 0 | 0 | 9 | 536.6813 | 0.741957 |
| 2305 | −0.3692 | −0.49878 | 0.458935 | 0 | 0.257995 | 0 | 9 | 533.831 | 0.746244 |
| 2306 | −0.36789 | −0.4958 | 0.464972 | 0 | 0 | 0.255069 | 9 | 534.0902 | 0.745937 |
| 2307 | −0.36683 | −0.32775 | 0 | 0.247282 | 0.215748 | 0 | 9 | 548.4099 | 0.727115 |
| 2308 | −0.36769 | −0.32552 | 0 | 0.266766 | 0 | 0.223296 | 9 | 548.1318 | 0.724808 |
| 2309 | −0.35346 | −0.32601 | 0 | 0 | 0.190682 | 0.171912 | 9 | 550.0337 | 0.724461 |
| 2310 | −0.36573 | 0 | 0.297911 | 0.217151 | 0.249518 | 0 | 9 | 540.0454 | 0.740201 |
| 2311 | −0.36751 | 0 | 0.304766 | 0.234822 | 0 | 0.26618 | 9 | 539.3587 | 0.74067 |
| 2312 | −0.35702 | 0 | 0.341819 | 0 | 0.21209 | 0.213821 | 9 | 539.2185 | 0.740242 |
| 2313 | −0.36116 | 0 | 0 | 0.256551 | 0.172681 | 0.188346 | 9 | 548.3558 | 0.72638 |
| 2314 | −0.3635 | 0 | 0.280897 | 0.247949 | 0 | 0 | 9 | 542.6456 | 0.73669 |
| 2315 | −0.36484 | −0.51223 | 0.448932 | 0 | 0 | 0 | 9 | 537.0253 | 0.742181 |
| 2316 | −0.36535 | −0.34122 | 0 | 0.272545 | 0 | 0 | 9 | 548.624 | 0.726013 |
| 2317 | −0.35247 | −0.33749 | 0 | 0 | 0.244058 | 0 | 9 | 549.0779 | 0.726829 |
| 2318 | −0.3509 | −0.33426 | 0 | 0 | 0 | 0.230316 | 9 | 547.8287 | 0.728279 |
| 2319 | −0.35435 | 0 | 0.326983 | 0 | 0.276248 | 0 | 9 | 540.9265 | 0.737384 |
| 2320 | −0.35429 | 0 | 0.335115 | 0 | 0 | 0.278056 | 9 | 538.5666 | 0.743508 |
| 2321 | −0.35918 | 0 | 0 | 0.257296 | 0.231108 | 0 | 9 | 547.9345 | 0.729259 |
| 2322 | −0.36046 | 0 | 0 | 0.27483 | 0 | 0.241127 | 9 | 545.6158 | 0.733076 |
| 2323 | −0.34604 | 0 | 0 | 0 | 0.20186 | 0.189915 | 9 | 548.4377 | 0.729095 |
| 2324 | −0.37665 | −0.49016 | 0.415897 | 0.213769 | 0 | 0 | 9 | 537.3471 | 0.74214 |
| 2325 | −0.3692 | −0.49878 | 0.458935 | 0 | 0.257995 | 0 | 9 | 535.4892 | 0.743345 |
| 2326 | −0.36789 | −0.4958 | 0.464972 | 0 | 0 | 0.255069 | 9 | 534.9223 | 0.744488 |
| 2327 | −0.36683 | −0.32775 | 0 | 0.247282 | 0.215748 | 0 | 9 | 549.2737 | 0.724175 |
| 2328 | −0.36769 | −0.32552 | 0 | 0.266766 | 0 | 0.223296 | 9 | 548.4682 | 0.724481 |
| 2329 | −0.35346 | −0.32601 | 0 | 0 | 0.190682 | 0.171912 | 9 | 550.7956 | 0.722787 |
| 2330 | −0.36573 | 0 | 0.297911 | 0.217151 | 0.249518 | 0 | 9 | 541.5616 | 0.736445 |
| 2331 | −0.36751 | 0 | 0.304766 | 0.234822 | 0 | 0.26618 | 9 | 540.1134 | 0.738996 |
| 2332 | −0.35702 | 0 | 0.341819 | 0 | 0.21209 | 0.213821 | 9 | 540.7424 | 0.738016 |
| 2333 | −0.36116 | 0 | 0 | 0.256551 | 0.172681 | 0.188346 | 9 | 549.2533 | 0.723951 |
| 2334 | −0.37665 | −0.49016 | 0.415897 | 0.213769 | 0 | 0 | 9 | 534.7631 | 0.746285 |
| 2335 | −0.3692 | −0.49878 | 0.458935 | 0 | 0.257995 | 0 | 9 | 532.3661 | 0.747632 |
| 2336 | −0.36789 | −0.4958 | 0.464972 | 0 | 0 | 0.255069 | 9 | 530.5945 | 0.751756 |
| 2337 | −0.36683 | −0.32775 | 0 | 0.247282 | 0.215748 | 0 | 9 | 545.7294 | 0.729993 |
| 2338 | −0.36769 | −0.32552 | 0 | 0.266766 | 0 | 0.223296 | 9 | 543.7601 | 0.732954 |
| 2339 | −0.35346 | −0.32601 | 0 | 0 | 0.190682 | 0.171912 | 9 | 546.3453 | 0.728687 |
| 2340 | −0.36573 | 0 | 0.297911 | 0.217151 | 0.249518 | 0 | 9 | 538.7872 | 0.740058 |
| 2341 | −0.36751 | 0 | 0.304766 | 0.234822 | 0 | 0.26618 | 9 | 536.0049 | 0.745713 |
| 2342 | −0.35702 | 0 | 0.341819 | 0 | 0.21209 | 0.213821 | 9 | 536.7025 | 0.742794 |
| 2343 | −0.36116 | 0 | 0 | 0.256551 | 0.172681 | 0.188346 | 9 | 544.8398 | 0.731974 |
| 2344 | −0.3784 | −0.48038 | 0.429094 | 0.184036 | 0.235336 | 0 | 9 | 534.2454 | 0.744937 |
| 2345 | −0.37944 | −0.47686 | 0.432955 | 0.204998 | 0 | 0.247828 | 9 | 533.2869 | 0.74608 |
| 2346 | −0.37057 | −0.48826 | 0.469023 | 0 | 0.198485 | 0.194003 | 9 | 533.2366 | 0.745692 |
| 2347 | −0.36821 | −0.31823 | 0 | 0.248938 | 0.16128 | 0.17389 | 9 | 547.8781 | 0.724236 |
| 2348 | −0.36844 | 0 | 0.31303 | 0.214129 | 0.186465 | 0.210249 | 9 | 539.0736 | 0.73918 |
| 2349 | −0.34954 | 0 | 0.311734 | 0 | 0 | 0 | 9 | 546.7363 | 0.731953 |
| 2350 | −0.34838 | −0.35299 | 0 | 0 | 0 | 0 | 9 | 553.5655 | 0.717724 |
| 2351 | −0.35731 | 0 | 0 | 0.283735 | 0 | 0 | 9 | 551.8214 | 0.724236 |
| 2352 | −0.34421 | 0 | 0 | 0 | 0.260301 | 0 | 9 | 552.0937 | 0.72342 |
| 2353 | −0.34349 | 0 | 0 | 0 | 0 | 0.25144 | 9 | 550.8901 | 0.725033 |
| 2354 | −0.34604 | 0 | 0 | 0 | 0.20186 | 0.189915 | 9 | 552.3348 | 0.721705 |
| 2355 | −0.3635 | 0 | 0.280897 | 0.247949 | 0 | 0 | 9 | 545.2216 | 0.732749 |
| 2356 | −0.36484 | −0.51223 | 0.448932 | 0 | 0 | 0 | 9 | 539.9433 | 0.736975 |
| 2357 | −0.36535 | −0.34122 | 0 | 0.272545 | 0 | 0 | 9 | 551.8773 | 0.720256 |
| 2358 | −0.35247 | −0.33749 | 0 | 0 | 0.244058 | 0 | 9 | 552.2186 | 0.723481 |
| 2359 | −0.3509 | −0.33426 | 0 | 0 | 0 | 0.230316 | 9 | 552.407 | 0.718582 |
| 2360 | −0.35435 | 0 | 0.326983 | 0 | 0.276248 | 0 | 9 | 543.339 | 0.734464 |
| 2361 | −0.35429 | 0 | 0.335115 | 0 | 0 | 0.278056 | 9 | 542.8041 | 0.735669 |
| 2362 | −0.35918 | 0 | 0 | 0.257296 | 0.231108 | 0 | 9 | 550.7961 | 0.723461 |
| 2363 | −0.36046 | 0 | 0 | 0.27483 | 0 | 0.241127 | 9 | 550.049 | 0.723685 |
| 2364 | −0.34604 | 0 | 0 | 0 | 0.20186 | 0.189915 | 9 | 548.9943 | 0.727095 |
| 2365 | −0.3635 | 0 | 0.280897 | 0.247949 | 0 | 0 | 9 | 543.5309 | 0.73571 |
| 2366 | −0.36484 | −0.51223 | 0.448932 | 0 | 0 | 0 | 9 | 537.917 | 0.740283 |
| 2367 | −0.36535 | −0.34122 | 0 | 0.272545 | 0 | 0 | 9 | 549.2563 | 0.724624 |
| 2368 | −0.35247 | −0.33749 | 0 | 0 | 0.244058 | 0 | 9 | 549.4166 | 0.725135 |
| 2369 | −0.3509 | −0.33426 | 0 | 0 | 0 | 0.230316 | 9 | 548.5886 | 0.724829 |
| 2370 | −0.35435 | 0 | 0.326983 | 0 | 0.276248 | 0 | 9 | 541.4189 | 0.736098 |
| 2371 | −0.35429 | 0 | 0.335115 | 0 | 0 | 0.278056 | 9 | 539.6745 | 0.74114 |
| 2372 | −0.35918 | 0 | 0 | 0.257296 | 0.231108 | 0 | 9 | 548.3227 | 0.726666 |
| 2373 | −0.36046 | 0 | 0 | 0.27483 | 0 | 0.241127 | 9 | 546.4529 | 0.731137 |
| 2374 | −0.37665 | −0.49016 | 0.415897 | 0.213769 | 0 | 0 | 9 | 537.6397 | 0.740262 |
| 2375 | −0.3692 | −0.49878 | 0.458935 | 0 | 0.257995 | 0 | 9 | 535.1723 | 0.74461 |
| 2376 | −0.36789 | −0.4958 | 0.464972 | 0 | 0 | 0.255069 | 9 | 535.1401 | 0.744284 |
| 2377 | −0.36683 | −0.32775 | 0 | 0.247282 | 0.215748 | 0 | 9 | 548.8946 | 0.727482 |
| 2378 | −0.36769 | −0.32552 | 0 | 0.266766 | 0 | 0.223296 | 9 | 548.4365 | 0.724257 |
| 2379 | −0.35346 | −0.32601 | 0 | 0 | 0.190682 | 0.171912 | 9 | 550.5156 | 0.723502 |
| 2380 | −0.36573 | 0 | 0.297911 | 0.217151 | 0.249518 | 0 | 9 | 541.0873 | 0.738609 |
| 2381 | −0.36751 | 0 | 0.304766 | 0.234822 | 0 | 0.26618 | 9 | 540.0795 | 0.739997 |
| 2382 | −0.35702 | 0 | 0.341819 | 0 | 0.21209 | 0.213821 | 9 | 540.3077 | 0.738935 |
| 2383 | −0.36116 | 0 | 0 | 0.256551 | 0.172681 | 0.188346 | 9 | 548.7535 | 0.725972 |
| 2384 | −0.34604 | 0 | 0 | 0 | 0.20186 | 0.189915 | 9 | 549.4672 | 0.725788 |
| 2385 | −0.36484 | −0.51223 | 0.448932 | 0 | 0 | 0 | 9 | 538.4061 | 0.739017 |
| 2386 | −0.3635 | 0 | 0.280897 | 0.247949 | 0 | 0 | 9 | 543.8657 | 0.734668 |
| 2387 | −0.36535 | −0.34122 | 0 | 0.272545 | 0 | 0 | 9 | 549.2935 | 0.724522 |
| 2388 | −0.35247 | −0.33749 | 0 | 0 | 0.244058 | 0 | 9 | 549.9937 | 0.725563 |
| 2389 | −0.3509 | −0.33426 | 0 | 0 | 0 | 0.230316 | 9 | 548.5746 | 0.725911 |
| 2390 | −0.35435 | 0 | 0.326983 | 0 | 0.276248 | 0 | 9 | 542.6293 | 0.733158 |
| 2391 | −0.35429 | 0 | 0.335115 | 0 | 0 | 0.278056 | 9 | 540.0284 | 0.740262 |
| 2392 | −0.35918 | 0 | 0 | 0.257296 | 0.231108 | 0 | 9 | 548.9283 | 0.72587 |
| 2393 | −0.36046 | 0 | 0 | 0.27483 | 0 | 0.241127 | 9 | 546.4494 | 0.730728 |
| 2394 | −0.37665 | −0.49016 | 0.415897 | 0.213769 | 0 | 0 | 9 | 538.5955 | 0.738976 |
| 2395 | −0.3692 | −0.49878 | 0.458935 | 0 | 0.257995 | 0 | 9 | 537.1551 | 0.74016 |
| 2396 | −0.36789 | −0.4958 | 0.464972 | 0 | 0 | 0.255069 | 9 | 536.2427 | 0.743059 |
| 2397 | −0.36683 | −0.32775 | 0 | 0.247282 | 0.215748 | 0 | 9 | 549.9816 | 0.723522 |
| 2398 | −0.36769 | −0.32552 | 0 | 0.266766 | 0 | 0.223296 | 9 | 548.944 | 0.723216 |
| 2399 | −0.35346 | −0.32601 | 0 | 0 | 0.190682 | 0.171912 | 9 | 551.4939 | 0.720868 |
| 2400 | −0.36573 | 0 | 0.297911 | 0.217151 | 0.249518 | 0 | 9 | 542.8546 | 0.734158 |
| 2401 | −0.36751 | 0 | 0.304766 | 0.234822 | 0 | 0.26618 | 9 | 541.0414 | 0.736751 |
| 2402 | −0.35702 | 0 | 0.341819 | 0 | 0.21209 | 0.213821 | 9 | 542.0635 | 0.735077 |
| 2403 | −0.36116 | 0 | 0 | 0.256551 | 0.172681 | 0.188346 | 9 | 549.8159 | 0.72291 |
| 2404 | −0.37665 | −0.49016 | 0.415897 | 0.213769 | 0 | 0 | 9 | 535.9893 | 0.743631 |
| 2405 | −0.3692 | −0.49878 | 0.458935 | 0 | 0.257995 | 0 | 9 | 534.117 | 0.744488 |
| 2406 | −0.36789 | −0.4958 | 0.464972 | 0 | 0 | 0.255069 | 9 | 532.0532 | 0.748326 |
| 2407 | −0.36683 | −0.32775 | 0 | 0.247282 | 0.215748 | 0 | 9 | 546.4955 | 0.728442 |
| 2408 | −0.36769 | −0.32552 | 0 | 0.266766 | 0 | 0.223296 | 9 | 544.3758 | 0.731688 |
| 2409 | −0.35346 | −0.32601 | 0 | 0 | 0.190682 | 0.171912 | 9 | 547.151 | 0.727625 |
| 2410 | −0.36573 | 0 | 0.297911 | 0.217151 | 0.249518 | 0 | 9 | 540.1813 | 0.736975 |
| 2411 | −0.36751 | 0 | 0.304766 | 0.234822 | 0 | 0.26618 | 9 | 537.1434 | 0.744018 |
| 2412 | −0.35702 | 0 | 0.341819 | 0 | 0.21209 | 0.213821 | 9 | 538.2599 | 0.740548 |
| 2413 | −0.36116 | 0 | 0 | 0.256551 | 0.172681 | 0.188346 | 9 | 545.6253 | 0.729932 |
| 2414 | −0.3784 | −0.48038 | 0.429094 | 0.184036 | 0.235336 | 0 | 9 | 535.5543 | 0.743263 |
| 2415 | −0.37944 | −0.47686 | 0.432955 | 0.204998 | 0 | 0.247828 | 9 | 534.2166 | 0.746591 |
| 2416 | −0.37057 | −0.48826 | 0.469023 | 0 | 0.198485 | 0.194003 | 9 | 534.5662 | 0.744896 |
| 2417 | −0.36821 | −0.31823 | 0 | 0.248938 | 0.16128 | 0.17389 | 9 | 548.1926 | 0.724236 |
| 2418 | −0.36844 | 0 | 0.31303 | 0.214129 | 0.186465 | 0.210249 | 9 | 539.867 | 0.738057 |
| 2419 | −0.34604 | 0 | 0 | 0 | 0.20186 | 0.189915 | 9 | 548.6587 | 0.728401 |
| 2420 | −0.36484 | −0.51223 | 0.448932 | 0 | 0 | 0 | 9 | 538.1929 | 0.740568 |
| 2421 | −0.3635 | 0 | 0.280897 | 0.247949 | 0 | 0 | 9 | 543.3715 | 0.735424 |
| 2422 | −0.36535 | −0.34122 | 0 | 0.272545 | 0 | 0 | 9 | 549.0553 | 0.726441 |
| 2423 | −0.35247 | −0.33749 | 0 | 0 | 0.244058 | 0 | 9 | 549.363 | 0.726339 |
| 2424 | −0.3509 | −0.33426 | 0 | 0 | 0 | 0.230316 | 9 | 548.3655 | 0.727237 |
| 2425 | −0.35435 | 0 | 0.326983 | 0 | 0.276248 | 0 | 9 | 541.4198 | 0.736404 |
| 2426 | −0.35429 | 0 | 0.335115 | 0 | 0 | 0.278056 | 9 | 539.4815 | 0.742161 |
| 2427 | −0.35918 | 0 | 0 | 0.257296 | 0.231108 | 0 | 9 | 548.0033 | 0.729156 |
| 2428 | −0.36046 | 0 | 0 | 0.27483 | 0 | 0.241127 | 9 | 545.9466 | 0.731933 |
| 2429 | −0.37665 | −0.49016 | 0.415897 | 0.213769 | 0 | 0 | 9 | 537.55 | 0.741201 |
| 2430 | −0.3692 | −0.49878 | 0.458935 | 0 | 0.257995 | 0 | 9 | 535.1624 | 0.74461 |
| 2431 | −0.36789 | −0.4958 | 0.464972 | 0 | 0 | 0.255069 | 9 | 534.9902 | 0.744958 |
| 2432 | −0.36683 | −0.32775 | 0 | 0.247282 | 0.215748 | 0 | 9 | 548.4916 | 0.728095 |
| 2433 | −0.36769 | −0.32552 | 0 | 0.266766 | 0 | 0.223296 | 9 | 547.9255 | 0.725666 |
| 2434 | −0.35346 | −0.32601 | 0 | 0 | 0.190682 | 0.171912 | 9 | 550.0786 | 0.724808 |
| 2435 | −0.36573 | 0 | 0.297911 | 0.217151 | 0.249518 | 0 | 9 | 540.8459 | 0.737996 |
| 2436 | −0.36751 | 0 | 0.304766 | 0.234822 | 0 | 0.26618 | 9 | 539.7181 | 0.741038 |
| 2437 | −0.35702 | 0 | 0.341819 | 0 | 0.21209 | 0.213821 | 9 | 540.0368 | 0.739037 |
| 2438 | −0.36116 | 0 | 0 | 0.256551 | 0.172681 | 0.188346 | 9 | 548.2203 | 0.726666 |
| 2439 | −0.37665 | −0.49016 | 0.415897 | 0.213769 | 0 | 0 | 9 | 535.5142 | 0.745243 |
| 2440 | −0.3692 | −0.49878 | 0.458935 | 0 | 0.257995 | 0 | 9 | 532.9545 | 0.747366 |
| 2441 | −0.36789 | −0.4958 | 0.464972 | 0 | 0 | 0.255069 | 9 | 531.5372 | 0.750674 |
| 2442 | −0.36683 | −0.32775 | 0 | 0.247282 | 0.215748 | 0 | 9 | 545.6389 | 0.730096 |
| 2443 | −0.36769 | −0.32552 | 0 | 0.266766 | 0 | 0.223296 | 9 | 543.8978 | 0.733137 |
| 2444 | −0.35346 | −0.32601 | 0 | 0 | 0.190682 | 0.171912 | 9 | 546.4226 | 0.729197 |
| 2445 | −0.36573 | 0 | 0.297911 | 0.217151 | 0.249518 | 0 | 9 | 538.85 | 0.739935 |
| 2446 | −0.36751 | 0 | 0.304766 | 0.234822 | 0 | 0.26618 | 9 | 536.4135 | 0.746489 |
| 2447 | −0.35702 | 0 | 0.341819 | 0 | 0.21209 | 0.213821 | 9 | 537.0101 | 0.74459 |
| 2448 | −0.36116 | 0 | 0 | 0.256551 | 0.172681 | 0.188346 | 9 | 544.6504 | 0.732892 |
| 2449 | −0.3784 | −0.48038 | 0.429094 | 0.184036 | 0.235336 | 0 | 9 | 533.4474 | 0.747938 |
| 2450 | −0.37944 | −0.47686 | 0.432955 | 0.204998 | 0 | 0.247828 | 9 | 532.7827 | 0.749081 |
| 2451 | −0.37057 | −0.48826 | 0.469023 | 0 | 0.198485 | 0.194003 | 9 | 532.4595 | 0.747285 |
| 2452 | −0.36821 | −0.31823 | 0 | 0.248938 | 0.16128 | 0.17389 | 9 | 546.6598 | 0.727952 |
| 2453 | −0.36844 | 0 | 0.31303 | 0.214129 | 0.186465 | 0.210249 | 9 | 537.8174 | 0.742753 |
| 2454 | −0.37665 | −0.49016 | 0.415897 | 0.213769 | 0 | 0 | 9 | 535.9822 | 0.744141 |
| 2455 | −0.3692 | −0.49878 | 0.458935 | 0 | 0.257995 | 0 | 9 | 534.1581 | 0.744529 |
| 2456 | −0.36789 | −0.4958 | 0.464972 | 0 | 0 | 0.255069 | 9 | 532.0235 | 0.749 |
| 2457 | −0.36683 | −0.32775 | 0 | 0.247282 | 0.215748 | 0 | 9 | 546.1931 | 0.728789 |
| 2458 | −0.36769 | −0.32552 | 0 | 0.266766 | 0 | 0.223296 | 9 | 543.9613 | 0.733444 |
| 2459 | −0.35346 | −0.32601 | 0 | 0 | 0.190682 | 0.171912 | 9 | 546.8496 | 0.728993 |
| 2460 | −0.36573 | 0 | 0.297911 | 0.217151 | 0.249518 | 0 | 9 | 539.9577 | 0.737506 |
| 2461 | −0.36751 | 0 | 0.304766 | 0.234822 | 0 | 0.26618 | 9 | 536.8005 | 0.743426 |
| 2462 | −0.35702 | 0 | 0.341819 | 0 | 0.21209 | 0.213821 | 9 | 538.0236 | 0.741181 |
| 2463 | −0.36116 | 0 | 0 | 0.256551 | 0.172681 | 0.188346 | 9 | 545.1286 | 0.731504 |
| 2464 | −0.3784 | −0.48038 | 0.429094 | 0.184036 | 0.235336 | 0 | 9 | 535.4322 | 0.74459 |
| 2465 | −0.37944 | −0.47686 | 0.432955 | 0.204998 | 0 | 0.247828 | 9 | 534.0593 | 0.74657 |
| 2466 | −0.37057 | −0.48826 | 0.469023 | 0 | 0.198485 | 0.194003 | 9 | 534.4504 | 0.744978 |
| 2467 | −0.36821 | −0.31823 | 0 | 0.248938 | 0.16128 | 0.17389 | 9 | 547.8133 | 0.725359 |
| 2468 | −0.36844 | 0 | 0.31303 | 0.214129 | 0.186465 | 0.210249 | 9 | 539.7136 | 0.738833 |
| 2469 | −0.3784 | −0.48038 | 0.429094 | 0.184036 | 0.235336 | 0 | 9 | 532.2866 | 0.748959 |
| 2470 | −0.37944 | −0.47686 | 0.432955 | 0.204998 | 0 | 0.247828 | 9 | 529.6323 | 0.752838 |
| 2471 | −0.37057 | −0.48826 | 0.469023 | 0 | 0.198485 | 0.194003 | 9 | 530.1516 | 0.749551 |
| 2472 | −0.36821 | −0.31823 | 0 | 0.248938 | 0.16128 | 0.17389 | 9 | 543.077 | 0.732341 |
| 2473 | −0.36844 | 0 | 0.31303 | 0.214129 | 0.186465 | 0.210249 | 9 | 535.5286 | 0.744794 |
| 2474 | −0.38017 | −0.47149 | 0.43946 | 0.184707 | 0.175726 | 0.194494 | 9 | 532.8726 | 0.747101 |
| 2475 | −0.37665 | −0.49016 | 0.415897 | 0.213769 | 0 | 0 | 9 | 561.5751 | 0.706149 |
| 2476 | −0.3692 | −0.49878 | 0.458935 | 0 | 0.257995 | 0 | 9 | 558.1215 | 0.70966 |
| 2477 | −0.36789 | −0.4958 | 0.464972 | 0 | 0 | 0.255069 | 9 | 557.3042 | 0.708211 |
| 2478 | −0.36683 | −0.32775 | 0 | 0.247282 | 0.215748 | 0 | 9 | 568.9306 | 0.695207 |
| 2479 | −0.36769 | −0.32552 | 0 | 0.266766 | 0 | 0.223296 | 9 | 567.3655 | 0.694084 |
| 2480 | −0.35346 | −0.32601 | 0 | 0 | 0.190682 | 0.171912 | 9 | 570.3077 | 0.691103 |
| 2481 | −0.36573 | 0 | 0.297911 | 0.217151 | 0.249518 | 0 | 9 | 569.5228 | 0.687326 |
| 2482 | −0.36751 | 0 | 0.304766 | 0.234822 | 0 | 0.26618 | 9 | 567.533 | 0.691083 |
| 2483 | −0.35702 | 0 | 0.341819 | 0 | 0.21209 | 0.213821 | 9 | 568.0106 | 0.690838 |
| 2484 | −0.36116 | 0 | 0 | 0.256551 | 0.172681 | 0.188346 | 9 | 571.8997 | 0.683223 |
| 2485 | −0.3784 | −0.48038 | 0.429094 | 0.184036 | 0.235336 | 0 | 9 | 557.9278 | 0.707945 |
| 2486 | −0.37944 | −0.47686 | 0.432955 | 0.204998 | 0 | 0.247828 | 9 | 557.9897 | 0.70572 |
| 2487 | −0.37057 | −0.48826 | 0.469023 | 0 | 0.198485 | 0.194003 | 9 | 557.4863 | 0.706047 |
| 2488 | −0.36821 | −0.31823 | 0 | 0.248938 | 0.16128 | 0.17389 | 9 | 569.7407 | 0.689531 |
| 2489 | −0.36844 | 0 | 0.31303 | 0.214129 | 0.186465 | 0.210249 | 9 | 568.1067 | 0.690164 |
| 2490 | −0.3784 | −0.48038 | 0.429094 | 0.184036 | 0.235336 | 0 | 9 | 555.3168 | 0.714294 |
| 2491 | −0.37944 | −0.47686 | 0.432955 | 0.204998 | 0 | 0.247828 | 9 | 553.6426 | 0.715581 |
| 2492 | −0.37057 | −0.48826 | 0.469023 | 0 | 0.198485 | 0.194003 | 9 | 553.4807 | 0.712927 |
| 2493 | −0.36821 | −0.31823 | 0 | 0.248938 | 0.16128 | 0.17389 | 9 | 565.0835 | 0.696942 |
| 2494 | −0.36844 | 0 | 0.31303 | 0.214129 | 0.186465 | 0.210249 | 9 | 564.2835 | 0.696656 |
| 2495 | −0.38017 | −0.47149 | 0.43946 | 0.184707 | 0.175726 | 0.194494 | 9 | 554.8956 | 0.70966 |
| 2496 | −0.3784 | −0.48038 | 0.429094 | 0.184036 | 0.235336 | 0 | 9 | 556.0179 | 0.712069 |
| 2497 | −0.37944 | −0.47686 | 0.432955 | 0.204998 | 0 | 0.247828 | 9 | 553.7929 | 0.714539 |
| 2498 | −0.37057 | −0.48826 | 0.469023 | 0 | 0.198485 | 0.194003 | 9 | 554.0593 | 0.710171 |
| 2499 | −0.36821 | −0.31823 | 0 | 0.248938 | 0.16128 | 0.17389 | 9 | 565.2275 | 0.697983 |
| 2500 | −0.36844 | 0 | 0.31303 | 0.214129 | 0.186465 | 0.210249 | 9 | 564.6462 | 0.694349 |
| 2501 | −0.38017 | −0.47149 | 0.43946 | 0.184707 | 0.175726 | 0.194494 | 9 | 556.2141 | 0.706537 |
| 2502 | −0.38017 | −0.47149 | 0.43946 | 0.184707 | 0.175726 | 0.194494 | 9 | 551.2542 | 0.715662 |
| 2503 | −0.34954 | 0 | 0.311734 | 0 | 0 | 0 | 8 | 550.1207 | 0.721746 |
| 2504 | −0.34838 | −0.35299 | 0 | 0 | 0 | 0 | 8 | 538.6069 | 0.743079 |
| 2505 | −0.35731 | 0 | 0 | 0.283735 | 0 | 0 | 8 | 547.5647 | 0.729504 |
| 2506 | −0.34421 | 0 | 0 | 0 | 0.260301 | 0 | 8 | 545.7743 | 0.731014 |
| 2507 | −0.34349 | 0 | 0 | 0 | 0 | 0.25144 | 8 | 547.5462 | 0.729381 |
| 2508 | −0.34954 | 0 | 0.311734 | 0 | 0 | 0 | 8 | 549.5272 | 0.721501 |
| 2509 | −0.34838 | −0.35299 | 0 | 0 | 0 | 0 | 8 | 538.0845 | 0.743222 |
| 2510 | −0.35731 | 0 | 0 | 0.283735 | 0 | 0 | 8 | 547.0345 | 0.729381 |
| 2511 | −0.34421 | 0 | 0 | 0 | 0.260301 | 0 | 8 | 545.7782 | 0.727278 |
| 2512 | −0.34349 | 0 | 0 | 0 | 0 | 0.25144 | 8 | 546.8819 | 0.729054 |
| 2513 | −0.34954 | 0 | 0.311734 | 0 | 0 | 0 | 8 | 546.551 | 0.726584 |
| 2514 | −0.34838 | −0.35299 | 0 | 0 | 0 | 0 | 8 | 533.3915 | 0.749694 |
| 2515 | −0.35731 | 0 | 0 | 0.283735 | 0 | 0 | 8 | 543.2522 | 0.735281 |
| 2516 | −0.34421 | 0 | 0 | 0 | 0.260301 | 0 | 8 | 541.4705 | 0.736159 |
| 2517 | −0.34349 | 0 | 0 | 0 | 0 | 0.25144 | 8 | 541.1316 | 0.7382 |
| 2518 | −0.3635 | 0 | 0.280897 | 0.247949 | 0 | 0 | 8 | 546.863 | 0.727891 |
| 2519 | −0.36484 | −0.51223 | 0.448932 | 0 | 0 | 0 | 8 | 531.0235 | 0.751143 |
| 2520 | −0.36535 | −0.34122 | 0 | 0.272545 | 0 | 0 | 8 | 534.9439 | 0.749959 |
| 2521 | −0.35247 | −0.33749 | 0 | 0 | 0.244058 | 0 | 8 | 534.149 | 0.747938 |
| 2522 | −0.3509 | −0.33426 | 0 | 0 | 0 | 0.230316 | 8 | 535.5563 | 0.746346 |
| 2523 | −0.35435 | 0 | 0.326983 | 0 | 0.276248 | 0 | 8 | 544.2678 | 0.729626 |
| 2524 | −0.35429 | 0 | 0.335115 | 0 | 0 | 0.278056 | 8 | 545.5261 | 0.729606 |
| 2525 | −0.35918 | 0 | 0 | 0.257296 | 0.231108 | 0 | 8 | 543.2902 | 0.733341 |
| 2526 | −0.36046 | 0 | 0 | 0.27483 | 0 | 0.241127 | 8 | 543.7767 | 0.735301 |
| 2527 | −0.34604 | 0 | 0 | 0 | 0.20186 | 0.189915 | 8 | 544.7032 | 0.731729 |
| 2528 | −0.34954 | 0 | 0.311734 | 0 | 0 | 0 | 8 | 549.6602 | 0.721052 |
| 2529 | −0.34838 | −0.35299 | 0 | 0 | 0 | 0 | 8 | 538.1791 | 0.74212 |
| 2530 | −0.35731 | 0 | 0 | 0.283735 | 0 | 0 | 8 | 546.8685 | 0.729075 |
| 2531 | −0.34421 | 0 | 0 | 0 | 0.260301 | 0 | 8 | 545.1402 | 0.730892 |
| 2532 | −0.34349 | 0 | 0 | 0 | 0 | 0.25144 | 8 | 546.6121 | 0.730381 |
| 2533 | −0.34954 | 0 | 0.311734 | 0 | 0 | 0 | 8 | 547.2876 | 0.725196 |
| 2534 | −0.34838 | −0.35299 | 0 | 0 | 0 | 0 | 8 | 534.2079 | 0.749551 |
| 2535 | −0.35731 | 0 | 0 | 0.283735 | 0 | 0 | 8 | 543.7132 | 0.734383 |
| 2536 | −0.34421 | 0 | 0 | 0 | 0.260301 | 0 | 8 | 541.7729 | 0.73624 |
| 2537 | −0.34349 | 0 | 0 | 0 | 0 | 0.25144 | 8 | 541.6738 | 0.737261 |
| 2538 | −0.3635 | 0 | 0.280897 | 0.247949 | 0 | 0 | 8 | 546.5177 | 0.728585 |
| 2539 | −0.36484 | −0.51223 | 0.448932 | 0 | 0 | 0 | 8 | 530.8501 | 0.751409 |
| 2540 | −0.36535 | −0.34122 | 0 | 0.272545 | 0 | 0 | 8 | 534.5081 | 0.75049 |
| 2541 | −0.35247 | −0.33749 | 0 | 0 | 0.244058 | 0 | 8 | 533.1721 | 0.749898 |
| 2542 | −0.3509 | −0.33426 | 0 | 0 | 0 | 0.230316 | 8 | 535.0456 | 0.747305 |
| 2543 | −0.35435 | 0 | 0.326983 | 0 | 0.276248 | 0 | 8 | 543.3004 | 0.733301 |
| 2544 | −0.35429 | 0 | 0.335115 | 0 | 0 | 0.278056 | 8 | 545.0101 | 0.731096 |
| 2545 | −0.35918 | 0 | 0 | 0.257296 | 0.231108 | 0 | 8 | 542.2125 | 0.736057 |
| 2546 | −0.36046 | 0 | 0 | 0.27483 | 0 | 0.241127 | 8 | 543.0674 | 0.737649 |
| 2547 | −0.34604 | 0 | 0 | 0 | 0.20186 | 0.189915 | 8 | 543.6989 | 0.734138 |
| 2548 | −0.34954 | 0 | 0.311734 | 0 | 0 | 0 | 8 | 546.4846 | 0.726421 |
| 2549 | −0.34838 | −0.35299 | 0 | 0 | 0 | 0 | 8 | 533.4966 | 0.749265 |
| 2550 | −0.35731 | 0 | 0 | 0.283735 | 0 | 0 | 8 | 542.9477 | 0.735587 |
| 2551 | −0.34421 | 0 | 0 | 0 | 0.260301 | 0 | 8 | 541.2167 | 0.735505 |
| 2552 | −0.34349 | 0 | 0 | 0 | 0 | 0.25144 | 8 | 540.7104 | 0.738016 |
| 2553 | −0.3635 | 0 | 0.280897 | 0.247949 | 0 | 0 | 8 | 546.1217 | 0.728585 |
| 2554 | −0.36484 | −0.51223 | 0.448932 | 0 | 0 | 0 | 8 | 530.7164 | 0.751552 |
| 2555 | −0.36535 | −0.34122 | 0 | 0.272545 | 0 | 0 | 8 | 534.2246 | 0.750102 |
| 2556 | −0.35247 | −0.33749 | 0 | 0 | 0.244058 | 0 | 8 | 533.4167 | 0.74753 |
| 2557 | −0.3509 | −0.33426 | 0 | 0 | 0 | 0.230316 | 8 | 534.6332 | 0.748081 |
| 2558 | −0.35435 | 0 | 0.326983 | 0 | 0.276248 | 0 | 8 | 543.4934 | 0.73132 |
| 2559 | −0.35429 | 0 | 0.335115 | 0 | 0 | 0.278056 | 8 | 544.5728 | 0.73128 |
| 2560 | −0.35918 | 0 | 0 | 0.257296 | 0.231108 | 0 | 8 | 542.3556 | 0.733239 |
| 2561 | −0.36046 | 0 | 0 | 0.27483 | 0 | 0.241127 | 8 | 542.6811 | 0.736363 |
| 2562 | −0.34604 | 0 | 0 | 0 | 0.20186 | 0.189915 | 8 | 543.5981 | 0.732055 |
| 2563 | −0.3635 | 0 | 0.280897 | 0.247949 | 0 | 0 | 8 | 543.1741 | 0.732872 |
| 2564 | −0.36484 | −0.51223 | 0.448932 | 0 | 0 | 0 | 8 | 526.9151 | 0.756839 |
| 2565 | −0.36535 | −0.34122 | 0 | 0.272545 | 0 | 0 | 8 | 529.4413 | 0.757574 |
| 2566 | −0.35247 | −0.33749 | 0 | 0 | 0.244058 | 0 | 8 | 528.2043 | 0.755206 |
| 2567 | −0.3509 | −0.33426 | 0 | 0 | 0 | 0.230316 | 8 | 527.7586 | 0.757206 |
| 2568 | −0.35435 | 0 | 0.326983 | 0 | 0.276248 | 0 | 8 | 540.1484 | 0.736526 |
| 2569 | −0.35429 | 0 | 0.335115 | 0 | 0 | 0.278056 | 8 | 539.6773 | 0.738711 |
| 2570 | −0.35918 | 0 | 0 | 0.257296 | 0.231108 | 0 | 8 | 538.1 | 0.741324 |
| 2571 | −0.36046 | 0 | 0 | 0.27483 | 0 | 0.241127 | 8 | 536.801 | 0.745897 |
| 2572 | −0.34604 | 0 | 0 | 0 | 0.20186 | 0.189915 | 8 | 538.0009 | 0.74114 |
| 2573 | −0.37665 | −0.49016 | 0.415897 | 0.213769 | 0 | 0 | 8 | 528.4651 | 0.7569 |
| 2574 | −0.3692 | −0.49878 | 0.458935 | 0 | 0.257995 | 0 | 8 | 525.6204 | 0.757696 |
| 2575 | −0.36789 | −0.4958 | 0.464972 | 0 | 0 | 0.255069 | 8 | 527.0246 | 0.755083 |
| 2576 | −0.36683 | −0.32775 | 0 | 0.247282 | 0.215748 | 0 | 8 | 530.8833 | 0.753879 |
| 2577 | −0.36769 | −0.32552 | 0 | 0.266766 | 0 | 0.223296 | 8 | 531.4727 | 0.754675 |
| 2578 | −0.35346 | −0.32601 | 0 | 0 | 0.190682 | 0.171912 | 8 | 532.5497 | 0.74949 |
| 2579 | −0.36573 | 0 | 0.297911 | 0.217151 | 0.249518 | 0 | 8 | 541.5266 | 0.735301 |
| 2580 | −0.36751 | 0 | 0.304766 | 0.234822 | 0 | 0.26618 | 8 | 542.0326 | 0.736016 |
| 2581 | −0.35702 | 0 | 0.341819 | 0 | 0.21209 | 0.213821 | 8 | 541.7372 | 0.734607 |
| 2582 | −0.36116 | 0 | 0 | 0.256551 | 0.172681 | 0.188346 | 8 | 541.0053 | 0.737404 |
| 2583 | −0.34954 | 0 | 0.311734 | 0 | 0 | 0 | 8 | 553.911 | 0.711988 |
| 2584 | −0.34838 | −0.35299 | 0 | 0 | 0 | 0 | 8 | 542.2451 | 0.73526 |
| 2585 | −0.35731 | 0 | 0 | 0.283735 | 0 | 0 | 8 | 550.5375 | 0.720194 |
| 2586 | −0.34421 | 0 | 0 | 0 | 0.260301 | 0 | 8 | 549.3805 | 0.72144 |
| 2587 | −0.34349 | 0 | 0 | 0 | 0 | 0.25144 | 8 | 550.5711 | 0.719929 |
| 2588 | −0.34954 | 0 | 0.311734 | 0 | 0 | 0 | 8 | 551.1111 | 0.718459 |
| 2589 | −0.34838 | −0.35299 | 0 | 0 | 0 | 0 | 8 | 538.098 | 0.742365 |
| 2590 | −0.35731 | 0 | 0 | 0.283735 | 0 | 0 | 8 | 547.0923 | 0.728258 |
| 2591 | −0.34421 | 0 | 0 | 0 | 0.260301 | 0 | 8 | 545.6382 | 0.728422 |
| 2592 | −0.34349 | 0 | 0 | 0 | 0 | 0.25144 | 8 | 545.4292 | 0.729585 |
| 2593 | −0.3635 | 0 | 0.280897 | 0.247949 | 0 | 0 | 8 | 550.278 | 0.720623 |
| 2594 | −0.36484 | −0.51223 | 0.448932 | 0 | 0 | 0 | 8 | 535.4754 | 0.744284 |
| 2595 | −0.36535 | −0.34122 | 0 | 0.272545 | 0 | 0 | 8 | 538.0991 | 0.743651 |
| 2596 | −0.35247 | −0.33749 | 0 | 0 | 0.244058 | 0 | 8 | 537.3032 | 0.743712 |
| 2597 | −0.3509 | −0.33426 | 0 | 0 | 0 | 0.230316 | 8 | 538.831 | 0.74065 |
| 2598 | −0.35435 | 0 | 0.326983 | 0 | 0.276248 | 0 | 8 | 547.8258 | 0.72344 |
| 2599 | −0.35429 | 0 | 0.335115 | 0 | 0 | 0.278056 | 8 | 549.1228 | 0.721889 |
| 2600 | −0.35918 | 0 | 0 | 0.257296 | 0.231108 | 0 | 8 | 545.8891 | 0.730749 |
| 2601 | −0.36046 | 0 | 0 | 0.27483 | 0 | 0.241127 | 8 | 546.4431 | 0.730218 |
| 2602 | −0.34604 | 0 | 0 | 0 | 0.20186 | 0.189915 | 8 | 547.5383 | 0.726543 |
| 2603 | −0.34954 | 0 | 0.311734 | 0 | 0 | 0 | 8 | 550.8944 | 0.717214 |
| 2604 | −0.34838 | −0.35299 | 0 | 0 | 0 | 0 | 8 | 537.9163 | 0.743406 |
| 2605 | −0.35731 | 0 | 0 | 0.283735 | 0 | 0 | 8 | 546.8899 | 0.727074 |
| 2606 | −0.34421 | 0 | 0 | 0 | 0.260301 | 0 | 8 | 545.8735 | 0.726666 |
| 2607 | −0.34349 | 0 | 0 | 0 | 0 | 0.25144 | 8 | 545.1079 | 0.728585 |
| 2608 | −0.3635 | 0 | 0.280897 | 0.247949 | 0 | 0 | 8 | 550.5797 | 0.718622 |
| 2609 | −0.36484 | −0.51223 | 0.448932 | 0 | 0 | 0 | 8 | 536.05 | 0.74312 |
| 2610 | −0.36535 | −0.34122 | 0 | 0.272545 | 0 | 0 | 8 | 538.4856 | 0.742794 |
| 2611 | −0.35247 | −0.33749 | 0 | 0 | 0.244058 | 0 | 8 | 538.398 | 0.738854 |
| 2612 | −0.3509 | −0.33426 | 0 | 0 | 0 | 0.230316 | 8 | 539.1764 | 0.739262 |
| 2613 | −0.35435 | 0 | 0.326983 | 0 | 0.276248 | 0 | 8 | 548.8811 | 0.719439 |
| 2614 | −0.35429 | 0 | 0.335115 | 0 | 0 | 0.278056 | 8 | 549.4739 | 0.719949 |
| 2615 | −0.35918 | 0 | 0 | 0.257296 | 0.231108 | 0 | 8 | 546.8368 | 0.724298 |
| 2616 | −0.36046 | 0 | 0 | 0.27483 | 0 | 0.241127 | 8 | 546.784 | 0.727891 |
| 2617 | −0.34604 | 0 | 0 | 0 | 0.20186 | 0.189915 | 8 | 548.2999 | 0.722399 |
| 2618 | −0.3635 | 0 | 0.280897 | 0.247949 | 0 | 0 | 8 | 547.2034 | 0.72689 |
| 2619 | −0.36484 | −0.51223 | 0.448932 | 0 | 0 | 0 | 8 | 532.0125 | 0.750408 |
| 2620 | −0.36535 | −0.34122 | 0 | 0.272545 | 0 | 0 | 8 | 533.5519 | 0.751531 |
| 2621 | −0.35247 | −0.33749 | 0 | 0 | 0.244058 | 0 | 8 | 533.0135 | 0.749408 |
| 2622 | −0.3509 | −0.33426 | 0 | 0 | 0 | 0.230316 | 8 | 532.2705 | 0.750531 |
| 2623 | −0.35435 | 0 | 0.326983 | 0 | 0.276248 | 0 | 8 | 545.0696 | 0.727095 |
| 2624 | −0.35429 | 0 | 0.335115 | 0 | 0 | 0.278056 | 8 | 544.2737 | 0.727401 |
| 2625 | −0.35918 | 0 | 0 | 0.257296 | 0.231108 | 0 | 8 | 542.2792 | 0.735036 |
| 2626 | −0.36046 | 0 | 0 | 0.27483 | 0 | 0.241127 | 8 | 540.7522 | 0.7372 |
| 2627 | −0.34604 | 0 | 0 | 0 | 0.20186 | 0.189915 | 8 | 542.4572 | 0.73279 |
| 2628 | −0.37665 | −0.49016 | 0.415897 | 0.213769 | 0 | 0 | 8 | 533.2873 | 0.749347 |
| 2629 | −0.3692 | −0.49878 | 0.458935 | 0 | 0.257995 | 0 | 8 | 531.381 | 0.748428 |
| 2630 | −0.36789 | −0.4958 | 0.464972 | 0 | 0 | 0.255069 | 8 | 532.2028 | 0.747305 |
| 2631 | −0.36683 | −0.32775 | 0 | 0.247282 | 0.215748 | 0 | 8 | 535.2033 | 0.746917 |
| 2632 | −0.36769 | −0.32552 | 0 | 0.266766 | 0 | 0.223296 | 8 | 535.3532 | 0.747244 |
| 2633 | −0.35346 | −0.32601 | 0 | 0 | 0.190682 | 0.171912 | 8 | 537.0283 | 0.742508 |
| 2634 | −0.36573 | 0 | 0.297911 | 0.217151 | 0.249518 | 0 | 8 | 546.1971 | 0.726788 |
| 2635 | −0.36751 | 0 | 0.304766 | 0.234822 | 0 | 0.26618 | 8 | 546.2248 | 0.727829 |
| 2636 | −0.35702 | 0 | 0.341819 | 0 | 0.21209 | 0.213821 | 8 | 546.6514 | 0.723502 |
| 2637 | −0.36116 | 0 | 0 | 0.256551 | 0.172681 | 0.188346 | 8 | 544.9407 | 0.730218 |
| 2638 | −0.34954 | 0 | 0.311734 | 0 | 0 | 0 | 8 | 552.1467 | 0.716071 |
| 2639 | −0.34838 | −0.35299 | 0 | 0 | 0 | 0 | 8 | 539.2676 | 0.740201 |
| 2640 | −0.35731 | 0 | 0 | 0.283735 | 0 | 0 | 8 | 547.788 | 0.725727 |
| 2641 | −0.34421 | 0 | 0 | 0 | 0.260301 | 0 | 8 | 546.6104 | 0.725992 |
| 2642 | −0.34349 | 0 | 0 | 0 | 0 | 0.25144 | 8 | 546.1174 | 0.727932 |
| 2643 | −0.3635 | 0 | 0.280897 | 0.247949 | 0 | 0 | 8 | 550.5361 | 0.71901 |
| 2644 | −0.36484 | −0.51223 | 0.448932 | 0 | 0 | 0 | 8 | 536.212 | 0.743447 |
| 2645 | −0.36535 | −0.34122 | 0 | 0.272545 | 0 | 0 | 8 | 538.3188 | 0.74261 |
| 2646 | −0.35247 | −0.33749 | 0 | 0 | 0.244058 | 0 | 8 | 537.6749 | 0.741793 |
| 2647 | −0.3509 | −0.33426 | 0 | 0 | 0 | 0.230316 | 8 | 538.8755 | 0.740323 |
| 2648 | −0.35435 | 0 | 0.326983 | 0 | 0.276248 | 0 | 8 | 548.2288 | 0.722522 |
| 2649 | −0.35429 | 0 | 0.335115 | 0 | 0 | 0.278056 | 8 | 549.1927 | 0.721705 |
| 2650 | −0.35918 | 0 | 0 | 0.257296 | 0.231108 | 0 | 8 | 545.9745 | 0.729667 |
| 2651 | −0.36046 | 0 | 0 | 0.27483 | 0 | 0.241127 | 8 | 546.2208 | 0.728279 |
| 2652 | −0.34604 | 0 | 0 | 0 | 0.20186 | 0.189915 | 8 | 547.4953 | 0.724931 |
| 2653 | −0.3635 | 0 | 0.280897 | 0.247949 | 0 | 0 | 8 | 547.9175 | 0.724951 |
| 2654 | −0.36484 | −0.51223 | 0.448932 | 0 | 0 | 0 | 8 | 533.1154 | 0.74902 |
| 2655 | −0.36535 | −0.34122 | 0 | 0.272545 | 0 | 0 | 8 | 534.2783 | 0.750592 |
| 2656 | −0.35247 | −0.33749 | 0 | 0 | 0.244058 | 0 | 8 | 533.5521 | 0.747366 |
| 2657 | −0.3509 | −0.33426 | 0 | 0 | 0 | 0.230316 | 8 | 533.1311 | 0.749816 |
| 2658 | −0.35435 | 0 | 0.326983 | 0 | 0.276248 | 0 | 8 | 545.4999 | 0.727319 |
| 2659 | −0.35429 | 0 | 0.335115 | 0 | 0 | 0.278056 | 8 | 544.999 | 0.729034 |
| 2660 | −0.35918 | 0 | 0 | 0.257296 | 0.231108 | 0 | 8 | 542.464 | 0.735077 |
| 2661 | −0.36046 | 0 | 0 | 0.27483 | 0 | 0.241127 | 8 | 541.1518 | 0.737588 |
| 2662 | −0.34604 | 0 | 0 | 0 | 0.20186 | 0.189915 | 8 | 542.8692 | 0.733586 |
| 2663 | −0.37665 | −0.49016 | 0.415897 | 0.213769 | 0 | 0 | 8 | 532.9779 | 0.748714 |
| 2664 | −0.3692 | −0.49878 | 0.458935 | 0 | 0.257995 | 0 | 8 | 530.3319 | 0.750082 |
| 2665 | −0.36789 | −0.4958 | 0.464972 | 0 | 0 | 0.255069 | 8 | 531.6371 | 0.747734 |
| 2666 | −0.36683 | −0.32775 | 0 | 0.247282 | 0.215748 | 0 | 8 | 534.048 | 0.749265 |
| 2667 | −0.36769 | −0.32552 | 0 | 0.266766 | 0 | 0.223296 | 8 | 534.5507 | 0.749204 |
| 2668 | −0.35346 | −0.32601 | 0 | 0 | 0.190682 | 0.171912 | 8 | 535.938 | 0.745611 |
| 2669 | −0.36573 | 0 | 0.297911 | 0.217151 | 0.249518 | 0 | 8 | 545.1465 | 0.729156 |
| 2670 | −0.36751 | 0 | 0.304766 | 0.234822 | 0 | 0.26618 | 8 | 545.5163 | 0.728585 |
| 2671 | −0.35702 | 0 | 0.341819 | 0 | 0.21209 | 0.213821 | 8 | 545.5826 | 0.726707 |
| 2672 | −0.36116 | 0 | 0 | 0.256551 | 0.172681 | 0.188346 | 8 | 543.7183 | 0.733301 |
| 2673 | −0.3635 | 0 | 0.280897 | 0.247949 | 0 | 0 | 8 | 547.8207 | 0.724134 |
| 2674 | −0.36484 | −0.51223 | 0.448932 | 0 | 0 | 0 | 8 | 533.1528 | 0.748755 |
| 2675 | −0.36535 | −0.34122 | 0 | 0.272545 | 0 | 0 | 8 | 534.235 | 0.749857 |
| 2676 | −0.35247 | −0.33749 | 0 | 0 | 0.244058 | 0 | 8 | 533.8541 | 0.746652 |
| 2677 | −0.3509 | −0.33426 | 0 | 0 | 0 | 0.230316 | 8 | 532.9723 | 0.748938 |
| 2678 | −0.35435 | 0 | 0.326983 | 0 | 0.276248 | 0 | 8 | 545.7843 | 0.725461 |
| 2679 | −0.35429 | 0 | 0.335115 | 0 | 0 | 0.278056 | 8 | 544.8441 | 0.727217 |
| 2680 | −0.35918 | 0 | 0 | 0.257296 | 0.231108 | 0 | 8 | 542.7195 | 0.733097 |
| 2681 | −0.36046 | 0 | 0 | 0.27483 | 0 | 0.241127 | 8 | 541.0015 | 0.736445 |
| 2682 | −0.34604 | 0 | 0 | 0 | 0.20186 | 0.189915 | 8 | 542.932 | 0.732749 |
| 2683 | −0.37665 | −0.49016 | 0.415897 | 0.213769 | 0 | 0 | 8 | 533.5205 | 0.74853 |
| 2684 | −0.3692 | −0.49878 | 0.458935 | 0 | 0.257995 | 0 | 8 | 531.5971 | 0.74804 |
| 2685 | −0.36789 | −0.4958 | 0.464972 | 0 | 0 | 0.255069 | 8 | 532.2464 | 0.746897 |
| 2686 | −0.36683 | −0.32775 | 0 | 0.247282 | 0.215748 | 0 | 8 | 535.0827 | 0.747162 |
| 2687 | −0.36769 | −0.32552 | 0 | 0.266766 | 0 | 0.223296 | 8 | 535.0434 | 0.747673 |
| 2688 | −0.35346 | −0.32601 | 0 | 0 | 0.190682 | 0.171912 | 8 | 536.8036 | 0.74214 |
| 2689 | −0.36573 | 0 | 0.297911 | 0.217151 | 0.249518 | 0 | 8 | 546.1067 | 0.72587 |
| 2690 | −0.36751 | 0 | 0.304766 | 0.234822 | 0 | 0.26618 | 8 | 545.9486 | 0.728401 |
| 2691 | −0.35702 | 0 | 0.341819 | 0 | 0.21209 | 0.213821 | 8 | 546.4484 | 0.723992 |
| 2692 | −0.36116 | 0 | 0 | 0.256551 | 0.172681 | 0.188346 | 8 | 544.5271 | 0.729585 |
| 2693 | −0.37665 | −0.49016 | 0.415897 | 0.213769 | 0 | 0 | 8 | 529.7105 | 0.753593 |
| 2694 | −0.3692 | −0.49878 | 0.458935 | 0 | 0.257995 | 0 | 8 | 527.4345 | 0.753634 |
| 2695 | −0.36789 | −0.4958 | 0.464972 | 0 | 0 | 0.255069 | 8 | 526.4823 | 0.755696 |
| 2696 | −0.36683 | −0.32775 | 0 | 0.247282 | 0.215748 | 0 | 8 | 529.9396 | 0.756267 |
| 2697 | −0.36769 | −0.32552 | 0 | 0.266766 | 0 | 0.223296 | 8 | 528.1651 | 0.759187 |
| 2698 | −0.35346 | −0.32601 | 0 | 0 | 0.190682 | 0.171912 | 8 | 530.4356 | 0.752205 |
| 2699 | −0.36573 | 0 | 0.297911 | 0.217151 | 0.249518 | 0 | 8 | 542.5687 | 0.73228 |
| 2700 | −0.36751 | 0 | 0.304766 | 0.234822 | 0 | 0.26618 | 8 | 540.8873 | 0.736771 |
| 2701 | −0.35702 | 0 | 0.341819 | 0 | 0.21209 | 0.213821 | 8 | 541.6339 | 0.731341 |
| 2702 | −0.36116 | 0 | 0 | 0.256551 | 0.172681 | 0.188346 | 8 | 538.7918 | 0.738364 |
| 2703 | −0.3784 | −0.48038 | 0.429094 | 0.184036 | 0.235336 | 0 | 8 | 529.3822 | 0.752368 |
| 2704 | −0.37944 | −0.47686 | 0.432955 | 0.204998 | 0 | 0.247828 | 8 | 529.4649 | 0.752674 |
| 2705 | −0.37057 | −0.48826 | 0.469023 | 0 | 0.198485 | 0.194003 | 8 | 529.5121 | 0.749959 |
| 2706 | −0.36821 | −0.31823 | 0 | 0.248938 | 0.16128 | 0.17389 | 8 | 533.3703 | 0.749347 |
| 2707 | −0.36844 | 0 | 0.31303 | 0.214129 | 0.186465 | 0.210249 | 8 | 543.6346 | 0.731116 |
| 2708 | −0.34954 | 0 | 0.311734 | 0 | 0 | 0 | 8 | 578.5495 | 0.666503 |
| 2709 | −0.34838 | −0.35299 | 0 | 0 | 0 | 0 | 8 | 579.4711 | 0.663053 |
| 2710 | −0.35731 | 0 | 0 | 0.283735 | 0 | 0 | 8 | 581.4172 | 0.660522 |
| 2711 | −0.34421 | 0 | 0 | 0 | 0.260301 | 0 | 8 | 580.77 | 0.671056 |
| 2712 | −0.34349 | 0 | 0 | 0 | 0 | 0.25144 | 8 | 582.2221 | 0.661216 |
| 2713 | −0.34954 | 0 | 0.311734 | 0 | 0 | 0 | 8 | 576.9324 | 0.667667 |
| 2714 | −0.34838 | −0.35299 | 0 | 0 | 0 | 0 | 8 | 576.7384 | 0.666463 |
| 2715 | −0.35731 | 0 | 0 | 0.283735 | 0 | 0 | 8 | 579.1316 | 0.666973 |
| 2716 | −0.34421 | 0 | 0 | 0 | 0.260301 | 0 | 8 | 578.0839 | 0.667504 |
| 2717 | −0.34349 | 0 | 0 | 0 | 0 | 0.25144 | 8 | 577.9933 | 0.665626 |
| 2718 | −0.3635 | 0 | 0.280897 | 0.247949 | 0 | 0 | 8 | 574.6154 | 0.675547 |
| 2719 | −0.36484 | −0.51223 | 0.448932 | 0 | 0 | 0 | 8 | 562.6473 | 0.693492 |
| 2720 | −0.36535 | −0.34122 | 0 | 0.272545 | 0 | 0 | 8 | 574.3614 | 0.673628 |
| 2721 | −0.35247 | −0.33749 | 0 | 0 | 0.244058 | 0 | 8 | 573.9647 | 0.677895 |
| 2722 | −0.3509 | −0.33426 | 0 | 0 | 0 | 0.230316 | 8 | 575.6562 | 0.670158 |
| 2723 | −0.35435 | 0 | 0.326983 | 0 | 0.276248 | 0 | 8 | 570.9608 | 0.681937 |
| 2724 | −0.35429 | 0 | 0.335115 | 0 | 0 | 0.278056 | 8 | 572.5178 | 0.678466 |
| 2725 | −0.35918 | 0 | 0 | 0.257296 | 0.231108 | 0 | 8 | 576.4214 | 0.673363 |
| 2726 | −0.36046 | 0 | 0 | 0.27483 | 0 | 0.241127 | 8 | 577.0756 | 0.668198 |
| 2727 | −0.34604 | 0 | 0 | 0 | 0.20186 | 0.189915 | 8 | 579.0266 | 0.668443 |
| 2728 | −0.34954 | 0 | 0.311734 | 0 | 0 | 0 | 8 | 576.2433 | 0.670464 |
| 2729 | −0.34838 | −0.35299 | 0 | 0 | 0 | 0 | 8 | 575.9966 | 0.671689 |
| 2730 | −0.35731 | 0 | 0 | 0.283735 | 0 | 0 | 8 | 578.3282 | 0.668279 |
| 2731 | −0.34421 | 0 | 0 | 0 | 0.260301 | 0 | 8 | 577.7437 | 0.671342 |
| 2732 | −0.34349 | 0 | 0 | 0 | 0 | 0.25144 | 8 | 577.0232 | 0.668974 |
| 2733 | −0.3635 | 0 | 0.280897 | 0.247949 | 0 | 0 | 8 | 574.2519 | 0.674057 |
| 2734 | −0.36484 | −0.51223 | 0.448932 | 0 | 0 | 0 | 8 | 562.5996 | 0.694696 |
| 2735 | −0.36535 | −0.34122 | 0 | 0.272545 | 0 | 0 | 8 | 573.9069 | 0.675363 |
| 2736 | −0.35247 | −0.33749 | 0 | 0 | 0.244058 | 0 | 8 | 574.1172 | 0.680283 |
| 2737 | −0.3509 | −0.33426 | 0 | 0 | 0 | 0.230316 | 8 | 575.1235 | 0.672607 |
| 2738 | −0.35435 | 0 | 0.326983 | 0 | 0.276248 | 0 | 8 | 571.5058 | 0.680304 |
| 2739 | −0.35429 | 0 | 0.335115 | 0 | 0 | 0.278056 | 8 | 572.1755 | 0.67912 |
| 2740 | −0.35918 | 0 | 0 | 0.257296 | 0.231108 | 0 | 8 | 576.5657 | 0.672567 |
| 2741 | −0.36046 | 0 | 0 | 0.27483 | 0 | 0.241127 | 8 | 576.559 | 0.667626 |
| 2742 | −0.34604 | 0 | 0 | 0 | 0.20186 | 0.189915 | 8 | 578.9545 | 0.668565 |
| 2743 | −0.3635 | 0 | 0.280897 | 0.247949 | 0 | 0 | 8 | 572.1935 | 0.678956 |
| 2744 | −0.36484 | −0.51223 | 0.448932 | 0 | 0 | 0 | 8 | 559.9499 | 0.698943 |
| 2745 | −0.36535 | −0.34122 | 0 | 0.272545 | 0 | 0 | 8 | 570.6895 | 0.683386 |
| 2746 | −0.35247 | −0.33749 | 0 | 0 | 0.244058 | 0 | 8 | 570.3954 | 0.681427 |
| 2747 | −0.3509 | −0.33426 | 0 | 0 | 0 | 0.230316 | 8 | 569.9877 | 0.680896 |
| 2748 | −0.35435 | 0 | 0.326983 | 0 | 0.276248 | 0 | 8 | 568.8232 | 0.680916 |
| 2749 | −0.35429 | 0 | 0.335115 | 0 | 0 | 0.278056 | 8 | 567.7711 | 0.684877 |
| 2750 | −0.35918 | 0 | 0 | 0.257296 | 0.231108 | 0 | 8 | 573.282 | 0.67618 |
| 2751 | −0.36046 | 0 | 0 | 0.27483 | 0 | 0.241127 | 8 | 571.7102 | 0.67865 |
| 2752 | −0.34604 | 0 | 0 | 0 | 0.20186 | 0.189915 | 8 | 574.2372 | 0.672689 |
| 2753 | −0.37665 | −0.49016 | 0.415897 | 0.213769 | 0 | 0 | 8 | 559.9298 | 0.696697 |
| 2754 | −0.3692 | −0.49878 | 0.458935 | 0 | 0.257995 | 0 | 8 | 556.7967 | 0.701964 |
| 2755 | −0.36789 | −0.4958 | 0.464972 | 0 | 0 | 0.255069 | 8 | 557.7329 | 0.698657 |
| 2756 | −0.36683 | −0.32775 | 0 | 0.247282 | 0.215748 | 0 | 8 | 570.269 | 0.683162 |
| 2757 | −0.36769 | −0.32552 | 0 | 0.266766 | 0 | 0.223296 | 8 | 570.4414 | 0.677119 |
| 2758 | −0.35346 | −0.32601 | 0 | 0 | 0.190682 | 0.171912 | 8 | 572.7963 | 0.675935 |
| 2759 | −0.36573 | 0 | 0.297911 | 0.217151 | 0.249518 | 0 | 8 | 568.8815 | 0.685795 |
| 2760 | −0.36751 | 0 | 0.304766 | 0.234822 | 0 | 0.26618 | 8 | 568.8717 | 0.683427 |
| 2761 | −0.35702 | 0 | 0.341819 | 0 | 0.21209 | 0.213821 | 8 | 568.8444 | 0.682651 |
| 2762 | −0.36116 | 0 | 0 | 0.256551 | 0.172681 | 0.188346 | 8 | 574.8212 | 0.670995 |
| 2763 | −0.34954 | 0 | 0.311734 | 0 | 0 | 0 | 8 | 577.1799 | 0.66881 |
| 2764 | −0.34838 | −0.35299 | 0 | 0 | 0 | 0 | 8 | 577.0481 | 0.670096 |
| 2765 | −0.35731 | 0 | 0 | 0.283735 | 0 | 0 | 8 | 578.9753 | 0.666667 |
| 2766 | −0.34421 | 0 | 0 | 0 | 0.260301 | 0 | 8 | 578.1123 | 0.671362 |
| 2767 | −0.34349 | 0 | 0 | 0 | 0 | 0.25144 | 8 | 577.7219 | 0.669668 |
| 2768 | −0.3635 | 0 | 0.280897 | 0.247949 | 0 | 0 | 8 | 574.2266 | 0.674996 |
| 2769 | −0.36484 | −0.51223 | 0.448932 | 0 | 0 | 0 | 8 | 562.8721 | 0.694227 |
| 2770 | −0.36535 | −0.34122 | 0 | 0.272545 | 0 | 0 | 8 | 573.8914 | 0.675302 |
| 2771 | −0.35247 | −0.33749 | 0 | 0 | 0.244058 | 0 | 8 | 573.5775 | 0.682958 |
| 2772 | −0.3509 | −0.33426 | 0 | 0 | 0 | 0.230316 | 8 | 575.0058 | 0.674608 |
| 2773 | −0.35435 | 0 | 0.326983 | 0 | 0.276248 | 0 | 8 | 570.7087 | 0.681733 |
| 2774 | −0.35429 | 0 | 0.335115 | 0 | 0 | 0.278056 | 8 | 571.8916 | 0.681427 |
| 2775 | −0.35918 | 0 | 0 | 0.257296 | 0.231108 | 0 | 8 | 575.7551 | 0.674996 |
| 2776 | −0.36046 | 0 | 0 | 0.27483 | 0 | 0.241127 | 8 | 576.1216 | 0.67028 |
| 2777 | −0.34604 | 0 | 0 | 0 | 0.20186 | 0.189915 | 8 | 578.2185 | 0.670464 |
| 2778 | −0.3635 | 0 | 0.280897 | 0.247949 | 0 | 0 | 8 | 572.6368 | 0.678671 |
| 2779 | −0.36484 | −0.51223 | 0.448932 | 0 | 0 | 0 | 8 | 560.8161 | 0.696962 |
| 2780 | −0.36535 | −0.34122 | 0 | 0.272545 | 0 | 0 | 8 | 571.2038 | 0.681508 |
| 2781 | −0.35247 | −0.33749 | 0 | 0 | 0.244058 | 0 | 8 | 570.6404 | 0.684019 |
| 2782 | −0.3509 | −0.33426 | 0 | 0 | 0 | 0.230316 | 8 | 570.5771 | 0.680528 |
| 2783 | −0.35435 | 0 | 0.326983 | 0 | 0.276248 | 0 | 8 | 568.7861 | 0.68259 |
| 2784 | −0.35429 | 0 | 0.335115 | 0 | 0 | 0.278056 | 8 | 568.1821 | 0.685918 |
| 2785 | −0.35918 | 0 | 0 | 0.257296 | 0.231108 | 0 | 8 | 573.179 | 0.677384 |
| 2786 | −0.36046 | 0 | 0 | 0.27483 | 0 | 0.241127 | 8 | 571.8997 | 0.679732 |
| 2787 | −0.34604 | 0 | 0 | 0 | 0.20186 | 0.189915 | 8 | 574.3137 | 0.67469 |
| 2788 | −0.37665 | −0.49016 | 0.415897 | 0.213769 | 0 | 0 | 8 | 559.743 | 0.698534 |
| 2789 | −0.3692 | −0.49878 | 0.458935 | 0 | 0.257995 | 0 | 8 | 555.7668 | 0.704087 |
| 2790 | −0.36789 | −0.4958 | 0.464972 | 0 | 0 | 0.255069 | 8 | 557.2969 | 0.700372 |
| 2791 | −0.36683 | −0.32775 | 0 | 0.247282 | 0.215748 | 0 | 8 | 569.3351 | 0.686653 |
| 2792 | −0.36769 | −0.32552 | 0 | 0.266766 | 0 | 0.223296 | 8 | 569.8987 | 0.678507 |
| 2793 | −0.35346 | −0.32601 | 0 | 0 | 0.190682 | 0.171912 | 8 | 571.9627 | 0.680304 |
| 2794 | −0.36573 | 0 | 0.297911 | 0.217151 | 0.249518 | 0 | 8 | 567.7563 | 0.688939 |
| 2795 | −0.36751 | 0 | 0.304766 | 0.234822 | 0 | 0.26618 | 8 | 568.2369 | 0.685571 |
| 2796 | −0.35702 | 0 | 0.341819 | 0 | 0.21209 | 0.213821 | 8 | 567.6912 | 0.687551 |
| 2797 | −0.36116 | 0 | 0 | 0.256551 | 0.172681 | 0.188346 | 8 | 573.7466 | 0.675629 |
| 2798 | −0.3635 | 0 | 0.280897 | 0.247949 | 0 | 0 | 8 | 572.0824 | 0.67912 |
| 2799 | −0.36484 | −0.51223 | 0.448932 | 0 | 0 | 0 | 8 | 560.5326 | 0.697554 |
| 2800 | −0.36535 | −0.34122 | 0 | 0.272545 | 0 | 0 | 8 | 570.6181 | 0.684121 |
| 2801 | −0.35247 | −0.33749 | 0 | 0 | 0.244058 | 0 | 8 | 570.3608 | 0.686612 |
| 2802 | −0.3509 | −0.33426 | 0 | 0 | 0 | 0.230316 | 8 | 569.8722 | 0.683284 |
| 2803 | −0.35435 | 0 | 0.326983 | 0 | 0.276248 | 0 | 8 | 568.7488 | 0.682937 |
| 2804 | −0.35429 | 0 | 0.335115 | 0 | 0 | 0.278056 | 8 | 567.5361 | 0.688163 |
| 2805 | −0.35918 | 0 | 0 | 0.257296 | 0.231108 | 0 | 8 | 572.8656 | 0.679201 |
| 2806 | −0.36046 | 0 | 0 | 0.27483 | 0 | 0.241127 | 8 | 571.1072 | 0.680773 |
| 2807 | −0.34604 | 0 | 0 | 0 | 0.20186 | 0.189915 | 8 | 573.7876 | 0.677446 |
| 2808 | −0.37665 | −0.49016 | 0.415897 | 0.213769 | 0 | 0 | 8 | 559.7173 | 0.699841 |
| 2809 | −0.3692 | −0.49878 | 0.458935 | 0 | 0.257995 | 0 | 8 | 556.57 | 0.703985 |
| 2810 | −0.36789 | −0.4958 | 0.464972 | 0 | 0 | 0.255069 | 8 | 557.3485 | 0.70029 |
| 2811 | −0.36683 | −0.32775 | 0 | 0.247282 | 0.215748 | 0 | 8 | 569.5275 | 0.686265 |
| 2812 | −0.36769 | −0.32552 | 0 | 0.266766 | 0 | 0.223296 | 8 | 569.5503 | 0.680835 |
| 2813 | −0.35346 | −0.32601 | 0 | 0 | 0.190682 | 0.171912 | 8 | 571.9814 | 0.681018 |
| 2814 | −0.36573 | 0 | 0.297911 | 0.217151 | 0.249518 | 0 | 8 | 568.2474 | 0.686489 |
| 2815 | −0.36751 | 0 | 0.304766 | 0.234822 | 0 | 0.26618 | 8 | 568.0385 | 0.686347 |
| 2816 | −0.35702 | 0 | 0.341819 | 0 | 0.21209 | 0.213821 | 8 | 568.0973 | 0.685224 |
| 2817 | −0.36116 | 0 | 0 | 0.256551 | 0.172681 | 0.188346 | 8 | 573.8016 | 0.673322 |
| 2818 | −0.37665 | −0.49016 | 0.415897 | 0.213769 | 0 | 0 | 8 | 557.19 | 0.705639 |
| 2819 | −0.3692 | −0.49878 | 0.458935 | 0 | 0.257995 | 0 | 8 | 553.4482 | 0.707864 |
| 2820 | −0.36789 | −0.4958 | 0.464972 | 0 | 0 | 0.255069 | 8 | 552.5963 | 0.70866 |
| 2821 | −0.36683 | −0.32775 | 0 | 0.247282 | 0.215748 | 0 | 8 | 565.8775 | 0.692165 |
| 2822 | −0.36769 | −0.32552 | 0 | 0.266766 | 0 | 0.223296 | 8 | 564.34 | 0.691838 |
| 2823 | −0.35346 | −0.32601 | 0 | 0 | 0.190682 | 0.171912 | 8 | 567.0601 | 0.688429 |
| 2824 | −0.36573 | 0 | 0.297911 | 0.217151 | 0.249518 | 0 | 8 | 565.6402 | 0.687857 |
| 2825 | −0.36751 | 0 | 0.304766 | 0.234822 | 0 | 0.26618 | 8 | 563.6713 | 0.692655 |
| 2826 | −0.35702 | 0 | 0.341819 | 0 | 0.21209 | 0.213821 | 8 | 563.7919 | 0.68994 |
| 2827 | −0.36116 | 0 | 0 | 0.256551 | 0.172681 | 0.188346 | 8 | 569.0031 | 0.681467 |
| 2828 | −0.3784 | −0.48038 | 0.429094 | 0.184036 | 0.235336 | 0 | 8 | 554.677 | 0.706108 |
| 2829 | −0.37944 | −0.47686 | 0.432955 | 0.204998 | 0 | 0.247828 | 8 | 554.7933 | 0.703556 |
| 2830 | −0.37057 | −0.48826 | 0.469023 | 0 | 0.198485 | 0.194003 | 8 | 554.1687 | 0.703291 |
| 2831 | −0.36821 | −0.31823 | 0 | 0.248938 | 0.16128 | 0.17389 | 8 | 568.0097 | 0.683305 |
| 2832 | −0.36844 | 0 | 0.31303 | 0.214129 | 0.186465 | 0.210249 | 8 | 565.5176 | 0.689082 |
| 2833 | −0.34954 | 0 | 0.311734 | 0 | 0 | 0 | 8 | 578.4019 | 0.667136 |
| 2834 | −0.34838 | −0.35299 | 0 | 0 | 0 | 0 | 8 | 577.3636 | 0.66977 |
| 2835 | −0.35731 | 0 | 0 | 0.283735 | 0 | 0 | 8 | 579.6634 | 0.666218 |
| 2836 | −0.34421 | 0 | 0 | 0 | 0.260301 | 0 | 8 | 578.9771 | 0.671301 |
| 2837 | −0.34349 | 0 | 0 | 0 | 0 | 0.25144 | 8 | 578.5065 | 0.668096 |
| 2838 | −0.3635 | 0 | 0.280897 | 0.247949 | 0 | 0 | 8 | 575.7717 | 0.673526 |
| 2839 | −0.36484 | −0.51223 | 0.448932 | 0 | 0 | 0 | 8 | 564.3864 | 0.69339 |
| 2840 | −0.36535 | −0.34122 | 0 | 0.272545 | 0 | 0 | 8 | 574.668 | 0.677344 |
| 2841 | −0.35247 | −0.33749 | 0 | 0 | 0.244058 | 0 | 8 | 574.6077 | 0.681406 |
| 2842 | −0.3509 | −0.33426 | 0 | 0 | 0 | 0.230316 | 8 | 575.8825 | 0.671423 |
| 2843 | −0.35435 | 0 | 0.326983 | 0 | 0.276248 | 0 | 8 | 572.7938 | 0.680651 |
| 2844 | −0.35429 | 0 | 0.335115 | 0 | 0 | 0.278056 | 8 | 573.7515 | 0.676568 |
| 2845 | −0.35918 | 0 | 0 | 0.257296 | 0.231108 | 0 | 8 | 577.0053 | 0.673628 |
| 2846 | −0.36046 | 0 | 0 | 0.27483 | 0 | 0.241127 | 8 | 577.2216 | 0.667279 |
| 2847 | −0.34604 | 0 | 0 | 0 | 0.20186 | 0.189915 | 8 | 579.4822 | 0.671607 |
| 2848 | −0.3635 | 0 | 0.280897 | 0.247949 | 0 | 0 | 8 | 573.8045 | 0.678038 |
| 2849 | −0.36484 | −0.51223 | 0.448932 | 0 | 0 | 0 | 8 | 561.9584 | 0.699106 |
| 2850 | −0.36535 | −0.34122 | 0 | 0.272545 | 0 | 0 | 8 | 571.6383 | 0.684142 |
| 2851 | −0.35247 | −0.33749 | 0 | 0 | 0.244058 | 0 | 8 | 571.2369 | 0.684346 |
| 2852 | −0.3509 | −0.33426 | 0 | 0 | 0 | 0.230316 | 8 | 571.0648 | 0.679344 |
| 2853 | −0.35435 | 0 | 0.326983 | 0 | 0.276248 | 0 | 8 | 570.4245 | 0.680569 |
| 2854 | −0.35429 | 0 | 0.335115 | 0 | 0 | 0.278056 | 8 | 569.6833 | 0.682019 |
| 2855 | −0.35918 | 0 | 0 | 0.257296 | 0.231108 | 0 | 8 | 574.0543 | 0.677139 |
| 2856 | −0.36046 | 0 | 0 | 0.27483 | 0 | 0.241127 | 8 | 572.7132 | 0.677425 |
| 2857 | −0.34604 | 0 | 0 | 0 | 0.20186 | 0.189915 | 8 | 575.1813 | 0.673914 |
| 2858 | −0.37665 | −0.49016 | 0.415897 | 0.213769 | 0 | 0 | 8 | 561.1218 | 0.69835 |
| 2859 | −0.3692 | −0.49878 | 0.458935 | 0 | 0.257995 | 0 | 8 | 557.7104 | 0.704312 |
| 2860 | −0.36789 | −0.4958 | 0.464972 | 0 | 0 | 0.255069 | 8 | 558.9476 | 0.69833 |
| 2861 | −0.36683 | −0.32775 | 0 | 0.247282 | 0.215748 | 0 | 8 | 570.2432 | 0.686551 |
| 2862 | −0.36769 | −0.32552 | 0 | 0.266766 | 0 | 0.223296 | 8 | 570.6557 | 0.680283 |
| 2863 | −0.35346 | −0.32601 | 0 | 0 | 0.190682 | 0.171912 | 8 | 572.8809 | 0.680508 |
| 2864 | −0.36573 | 0 | 0.297911 | 0.217151 | 0.249518 | 0 | 8 | 569.5582 | 0.68702 |
| 2865 | −0.36751 | 0 | 0.304766 | 0.234822 | 0 | 0.26618 | 8 | 569.7851 | 0.681855 |
| 2866 | −0.35702 | 0 | 0.341819 | 0 | 0.21209 | 0.213821 | 8 | 569.6554 | 0.684611 |
| 2867 | −0.36116 | 0 | 0 | 0.256551 | 0.172681 | 0.188346 | 8 | 574.8184 | 0.674363 |
| 2868 | −0.3635 | 0 | 0.280897 | 0.247949 | 0 | 0 | 8 | 573.606 | 0.677323 |
| 2869 | −0.36484 | −0.51223 | 0.448932 | 0 | 0 | 0 | 8 | 562.0366 | 0.69929 |
| 2870 | −0.36535 | −0.34122 | 0 | 0.272545 | 0 | 0 | 8 | 571.343 | 0.684877 |
| 2871 | −0.35247 | −0.33749 | 0 | 0 | 0.244058 | 0 | 8 | 571.4837 | 0.686428 |
| 2872 | −0.3509 | −0.33426 | 0 | 0 | 0 | 0.230316 | 8 | 570.6822 | 0.682733 |
| 2873 | −0.35435 | 0 | 0.326983 | 0 | 0.276248 | 0 | 8 | 571.0023 | 0.678875 |
| 2874 | −0.35429 | 0 | 0.335115 | 0 | 0 | 0.278056 | 8 | 569.4668 | 0.683162 |
| 2875 | −0.35918 | 0 | 0 | 0.257296 | 0.231108 | 0 | 8 | 574.2613 | 0.676139 |
| 2876 | −0.36046 | 0 | 0 | 0.27483 | 0 | 0.241127 | 8 | 572.2965 | 0.677609 |
| 2877 | −0.34604 | 0 | 0 | 0 | 0.20186 | 0.189915 | 8 | 575.1928 | 0.672832 |
| 2878 | −0.37665 | −0.49016 | 0.415897 | 0.213769 | 0 | 0 | 8 | 561.5801 | 0.699045 |
| 2879 | −0.3692 | −0.49878 | 0.458935 | 0 | 0.257995 | 0 | 8 | 559.1879 | 0.701903 |
| 2880 | −0.36789 | −0.4958 | 0.464972 | 0 | 0 | 0.255069 | 8 | 559.5213 | 0.697738 |
| 2881 | −0.36683 | −0.32775 | 0 | 0.247282 | 0.215748 | 0 | 8 | 570.9568 | 0.686551 |
| 2882 | −0.36769 | −0.32552 | 0 | 0.266766 | 0 | 0.223296 | 8 | 570.7133 | 0.679793 |
| 2883 | −0.35346 | −0.32601 | 0 | 0 | 0.190682 | 0.171912 | 8 | 573.42 | 0.679671 |
| 2884 | −0.36573 | 0 | 0.297911 | 0.217151 | 0.249518 | 0 | 8 | 570.636 | 0.682917 |
| 2885 | −0.36751 | 0 | 0.304766 | 0.234822 | 0 | 0.26618 | 8 | 570.0616 | 0.680528 |
| 2886 | −0.35702 | 0 | 0.341819 | 0 | 0.21209 | 0.213821 | 8 | 570.6571 | 0.681529 |
| 2887 | −0.36116 | 0 | 0 | 0.256551 | 0.172681 | 0.188346 | 8 | 575.3555 | 0.670811 |
| 2888 | −0.37665 | −0.49016 | 0.415897 | 0.213769 | 0 | 0 | 8 | 558.6701 | 0.706476 |
| 2889 | −0.3692 | −0.49878 | 0.458935 | 0 | 0.257995 | 0 | 8 | 555.7053 | 0.706639 |
| 2890 | −0.36789 | −0.4958 | 0.464972 | 0 | 0 | 0.255069 | 8 | 554.4235 | 0.707088 |
| 2891 | −0.36683 | −0.32775 | 0 | 0.247282 | 0.215748 | 0 | 8 | 566.9716 | 0.692124 |
| 2892 | −0.36769 | −0.32552 | 0 | 0.266766 | 0 | 0.223296 | 8 | 565.2255 | 0.69194 |
| 2893 | −0.35346 | −0.32601 | 0 | 0 | 0.190682 | 0.171912 | 8 | 568.1484 | 0.687 |
| 2894 | −0.36573 | 0 | 0.297911 | 0.217151 | 0.249518 | 0 | 8 | 567.6543 | 0.686387 |
| 2895 | −0.36751 | 0 | 0.304766 | 0.234822 | 0 | 0.26618 | 8 | 565.4111 | 0.690103 |
| 2896 | −0.35702 | 0 | 0.341819 | 0 | 0.21209 | 0.213821 | 8 | 566.0368 | 0.686714 |
| 2897 | −0.36116 | 0 | 0 | 0.256551 | 0.172681 | 0.188346 | 8 | 570.3196 | 0.678834 |
| 2898 | −0.3784 | −0.48038 | 0.429094 | 0.184036 | 0.235336 | 0 | 8 | 556.9548 | 0.704924 |
| 2899 | −0.37944 | −0.47686 | 0.432955 | 0.204998 | 0 | 0.247828 | 8 | 556.6484 | 0.70129 |
| 2900 | −0.37057 | −0.48826 | 0.469023 | 0 | 0.198485 | 0.194003 | 8 | 556.5784 | 0.700596 |
| 2901 | −0.36821 | −0.31823 | 0 | 0.248938 | 0.16128 | 0.17389 | 8 | 569.1585 | 0.683774 |
| 2902 | −0.36844 | 0 | 0.31303 | 0.214129 | 0.186465 | 0.210249 | 8 | 567.5864 | 0.686551 |
| 2903 | −0.34954 | 0 | 0.311734 | 0 | 0 | 0 | 8 | 507.8214 | 0.779949 |
| 2904 | −0.34838 | −0.35299 | 0 | 0 | 0 | 0 | 8 | 503.8728 | 0.784154 |
| 2905 | −0.35731 | 0 | 0 | 0.283735 | 0 | 0 | 8 | 508.0997 | 0.777131 |
| 2906 | −0.34421 | 0 | 0 | 0 | 0.260301 | 0 | 8 | 508.347 | 0.777703 |
| 2907 | −0.34349 | 0 | 0 | 0 | 0 | 0.25144 | 8 | 508.0212 | 0.779214 |
| 2908 | −0.34954 | 0 | 0.311734 | 0 | 0 | 0 | 8 | 506.4817 | 0.781684 |
| 2909 | −0.34838 | −0.35299 | 0 | 0 | 0 | 0 | 8 | 502.6804 | 0.784501 |
| 2910 | −0.35731 | 0 | 0 | 0.283735 | 0 | 0 | 8 | 506.7798 | 0.780581 |
| 2911 | −0.34421 | 0 | 0 | 0 | 0.260301 | 0 | 8 | 506.5008 | 0.780704 |
| 2912 | −0.34349 | 0 | 0 | 0 | 0 | 0.25144 | 8 | 506.6965 | 0.781806 |
| 2913 | −0.34954 | 0 | 0.311734 | 0 | 0 | 0 | 8 | 507.0544 | 0.780745 |
| 2914 | −0.34838 | −0.35299 | 0 | 0 | 0 | 0 | 8 | 503.1031 | 0.784583 |
| 2915 | −0.35731 | 0 | 0 | 0.283735 | 0 | 0 | 8 | 507.3086 | 0.778295 |
| 2916 | −0.34421 | 0 | 0 | 0 | 0.260301 | 0 | 8 | 507.5428 | 0.777968 |
| 2917 | −0.34349 | 0 | 0 | 0 | 0 | 0.25144 | 8 | 507.1858 | 0.780418 |
| 2918 | −0.34954 | 0 | 0.311734 | 0 | 0 | 0 | 8 | 503.7176 | 0.784848 |
| 2919 | −0.34838 | −0.35299 | 0 | 0 | 0 | 0 | 8 | 498.3064 | 0.790932 |
| 2920 | −0.35731 | 0 | 0 | 0.283735 | 0 | 0 | 8 | 503.1981 | 0.783562 |
| 2921 | −0.34421 | 0 | 0 | 0 | 0.260301 | 0 | 8 | 503.1293 | 0.785011 |
| 2922 | −0.34349 | 0 | 0 | 0 | 0 | 0.25144 | 8 | 501.7895 | 0.787788 |
| 2923 | −0.3635 | 0 | 0.280897 | 0.247949 | 0 | 0 | 8 | 505.3834 | 0.781112 |
| 2924 | −0.36484 | −0.51223 | 0.448932 | 0 | 0 | 0 | 8 | 495.5504 | 0.794525 |
| 2925 | −0.36535 | −0.34122 | 0 | 0.272545 | 0 | 0 | 8 | 500.9503 | 0.786236 |
| 2926 | −0.35247 | −0.33749 | 0 | 0 | 0.244058 | 0 | 8 | 501.339 | 0.785624 |
| 2927 | −0.3509 | −0.33426 | 0 | 0 | 0 | 0.230316 | 8 | 501.2129 | 0.787155 |
| 2928 | −0.35435 | 0 | 0.326983 | 0 | 0.276248 | 0 | 8 | 504.7013 | 0.782786 |
| 2929 | −0.35429 | 0 | 0.335115 | 0 | 0 | 0.278056 | 8 | 504.188 | 0.783644 |
| 2930 | −0.35918 | 0 | 0 | 0.257296 | 0.231108 | 0 | 8 | 505.8651 | 0.77954 |
| 2931 | −0.36046 | 0 | 0 | 0.27483 | 0 | 0.241127 | 8 | 505.2083 | 0.781194 |
| 2932 | −0.34604 | 0 | 0 | 0 | 0.20186 | 0.189915 | 8 | 506.6481 | 0.780112 |
| 2933 | −0.34954 | 0 | 0.311734 | 0 | 0 | 0 | 8 | 509.4783 | 0.778479 |
| 2934 | −0.34838 | −0.35299 | 0 | 0 | 0 | 0 | 8 | 505.562 | 0.780786 |
| 2935 | −0.35731 | 0 | 0 | 0.283735 | 0 | 0 | 8 | 509.2552 | 0.776172 |
| 2936 | −0.34421 | 0 | 0 | 0 | 0.260301 | 0 | 8 | 509.3005 | 0.777478 |
| 2937 | −0.34349 | 0 | 0 | 0 | 0 | 0.25144 | 8 | 509.2864 | 0.777274 |
| 2938 | −0.34954 | 0 | 0.311734 | 0 | 0 | 0 | 8 | 510.558 | 0.775825 |
| 2939 | −0.34838 | −0.35299 | 0 | 0 | 0 | 0 | 8 | 506.478 | 0.778989 |
| 2940 | −0.35731 | 0 | 0 | 0.283735 | 0 | 0 | 8 | 510.2604 | 0.773742 |
| 2941 | −0.34421 | 0 | 0 | 0 | 0.260301 | 0 | 8 | 510.8403 | 0.774069 |
| 2942 | −0.34349 | 0 | 0 | 0 | 0 | 0.25144 | 8 | 510.2487 | 0.775376 |
| 2943 | −0.34954 | 0 | 0.311734 | 0 | 0 | 0 | 8 | 507.3058 | 0.781316 |
| 2944 | −0.34838 | −0.35299 | 0 | 0 | 0 | 0 | 8 | 501.8938 | 0.786277 |
| 2945 | −0.35731 | 0 | 0 | 0.283735 | 0 | 0 | 8 | 506.3218 | 0.780847 |
| 2946 | −0.34421 | 0 | 0 | 0 | 0.260301 | 0 | 8 | 506.6609 | 0.7815 |
| 2947 | −0.34349 | 0 | 0 | 0 | 0 | 0.25144 | 8 | 505.2225 | 0.782725 |
| 2948 | −0.3635 | 0 | 0.280897 | 0.247949 | 0 | 0 | 8 | 508.3847 | 0.777927 |
| 2949 | −0.36484 | −0.51223 | 0.448932 | 0 | 0 | 0 | 8 | 499.5286 | 0.79036 |
| 2950 | −0.36535 | −0.34122 | 0 | 0.272545 | 0 | 0 | 8 | 503.8426 | 0.781439 |
| 2951 | −0.35247 | −0.33749 | 0 | 0 | 0.244058 | 0 | 8 | 504.6096 | 0.780745 |
| 2952 | −0.3509 | −0.33426 | 0 | 0 | 0 | 0.230316 | 8 | 504.2015 | 0.78148 |
| 2953 | −0.35435 | 0 | 0.326983 | 0 | 0.276248 | 0 | 8 | 508.1756 | 0.777233 |
| 2954 | −0.35429 | 0 | 0.335115 | 0 | 0 | 0.278056 | 8 | 507.3568 | 0.778805 |
| 2955 | −0.35918 | 0 | 0 | 0.257296 | 0.231108 | 0 | 8 | 508.6136 | 0.775804 |
| 2956 | −0.36046 | 0 | 0 | 0.27483 | 0 | 0.241127 | 8 | 507.671 | 0.777703 |
| 2957 | −0.34604 | 0 | 0 | 0 | 0.20186 | 0.189915 | 8 | 509.4717 | 0.775661 |
| 2958 | −0.34954 | 0 | 0.311734 | 0 | 0 | 0 | 8 | 509.1056 | 0.778356 |
| 2959 | −0.34838 | −0.35299 | 0 | 0 | 0 | 0 | 8 | 505.196 | 0.781194 |
| 2960 | −0.35731 | 0 | 0 | 0.283735 | 0 | 0 | 8 | 508.7928 | 0.77709 |
| 2961 | −0.34421 | 0 | 0 | 0 | 0.260301 | 0 | 8 | 508.8421 | 0.778172 |
| 2962 | −0.34349 | 0 | 0 | 0 | 0 | 0.25144 | 8 | 508.7353 | 0.777948 |
| 2963 | −0.34954 | 0 | 0.311734 | 0 | 0 | 0 | 8 | 506.595 | 0.782745 |
| 2964 | −0.34838 | −0.35299 | 0 | 0 | 0 | 0 | 8 | 501.4731 | 0.786543 |
| 2965 | −0.35731 | 0 | 0 | 0.283735 | 0 | 0 | 8 | 505.6012 | 0.782358 |
| 2966 | −0.34421 | 0 | 0 | 0 | 0.260301 | 0 | 8 | 505.6005 | 0.782888 |
| 2967 | −0.34349 | 0 | 0 | 0 | 0 | 0.25144 | 8 | 504.5499 | 0.78346 |
| 2968 | −0.3635 | 0 | 0.280897 | 0.247949 | 0 | 0 | 8 | 506.7383 | 0.78052 |
| 2969 | −0.36484 | −0.51223 | 0.448932 | 0 | 0 | 0 | 8 | 497.9108 | 0.790442 |
| 2970 | −0.36535 | −0.34122 | 0 | 0.272545 | 0 | 0 | 8 | 502.2996 | 0.784195 |
| 2971 | −0.35247 | −0.33749 | 0 | 0 | 0.244058 | 0 | 8 | 502.4812 | 0.784072 |
| 2972 | −0.3509 | −0.33426 | 0 | 0 | 0 | 0.230316 | 8 | 502.6246 | 0.784624 |
| 2973 | −0.35435 | 0 | 0.326983 | 0 | 0.276248 | 0 | 8 | 505.8343 | 0.781725 |
| 2974 | −0.35429 | 0 | 0.335115 | 0 | 0 | 0.278056 | 8 | 505.6067 | 0.782847 |
| 2975 | −0.35918 | 0 | 0 | 0.257296 | 0.231108 | 0 | 8 | 506.4425 | 0.780316 |
| 2976 | −0.36046 | 0 | 0 | 0.27483 | 0 | 0.241127 | 8 | 505.9827 | 0.780581 |
| 2977 | −0.34604 | 0 | 0 | 0 | 0.20186 | 0.189915 | 8 | 507.3891 | 0.779949 |
| 2978 | −0.34954 | 0 | 0.311734 | 0 | 0 | 0 | 8 | 507.043 | 0.781276 |
| 2979 | −0.34838 | −0.35299 | 0 | 0 | 0 | 0 | 8 | 501.7636 | 0.785828 |
| 2980 | −0.35731 | 0 | 0 | 0.283735 | 0 | 0 | 8 | 505.9863 | 0.780867 |
| 2981 | −0.34421 | 0 | 0 | 0 | 0.260301 | 0 | 8 | 506.345 | 0.781459 |
| 2982 | −0.34349 | 0 | 0 | 0 | 0 | 0.25144 | 8 | 504.8272 | 0.783133 |
| 2983 | −0.3635 | 0 | 0.280897 | 0.247949 | 0 | 0 | 8 | 507.9729 | 0.778132 |
| 2984 | −0.36484 | −0.51223 | 0.448932 | 0 | 0 | 0 | 8 | 499.234 | 0.790217 |
| 2985 | −0.36535 | −0.34122 | 0 | 0.272545 | 0 | 0 | 8 | 503.4206 | 0.7815 |
| 2986 | −0.35247 | −0.33749 | 0 | 0 | 0.244058 | 0 | 8 | 504.1821 | 0.781194 |
| 2987 | −0.3509 | −0.33426 | 0 | 0 | 0 | 0.230316 | 8 | 503.7123 | 0.78199 |
| 2988 | −0.35435 | 0 | 0.326983 | 0 | 0.276248 | 0 | 8 | 507.7775 | 0.778948 |
| 2989 | −0.35429 | 0 | 0.335115 | 0 | 0 | 0.278056 | 8 | 506.9204 | 0.779091 |
| 2990 | −0.35918 | 0 | 0 | 0.257296 | 0.231108 | 0 | 8 | 508.171 | 0.776233 |
| 2991 | −0.36046 | 0 | 0 | 0.27483 | 0 | 0.241127 | 8 | 507.2109 | 0.77756 |
| 2992 | −0.34604 | 0 | 0 | 0 | 0.20186 | 0.189915 | 8 | 508.9685 | 0.77607 |
| 2993 | −0.3635 | 0 | 0.280897 | 0.247949 | 0 | 0 | 8 | 504.5446 | 0.783787 |
| 2994 | −0.36484 | −0.51223 | 0.448932 | 0 | 0 | 0 | 8 | 495.2596 | 0.796015 |
| 2995 | −0.36535 | −0.34122 | 0 | 0.272545 | 0 | 0 | 8 | 498.5946 | 0.788645 |
| 2996 | −0.35247 | −0.33749 | 0 | 0 | 0.244058 | 0 | 8 | 499.1576 | 0.788155 |
| 2997 | −0.3509 | −0.33426 | 0 | 0 | 0 | 0.230316 | 8 | 497.6156 | 0.790605 |
| 2998 | −0.35435 | 0 | 0.326983 | 0 | 0.276248 | 0 | 8 | 504.0988 | 0.783684 |
| 2999 | −0.35429 | 0 | 0.335115 | 0 | 0 | 0.278056 | 8 | 502.3045 | 0.785277 |
| 3000 | −0.35918 | 0 | 0 | 0.257296 | 0.231108 | 0 | 8 | 503.763 | 0.78299 |
| 3001 | −0.36046 | 0 | 0 | 0.27483 | 0 | 0.241127 | 8 | 501.772 | 0.785358 |
| 3002 | −0.34604 | 0 | 0 | 0 | 0.20186 | 0.189915 | 8 | 503.8128 | 0.783827 |
| 3003 | −0.37665 | −0.49016 | 0.415897 | 0.213769 | 0 | 0 | 8 | 497.5003 | 0.79087 |
| 3004 | −0.3692 | −0.49878 | 0.458935 | 0 | 0.257995 | 0 | 8 | 497.1042 | 0.790728 |
| 3005 | −0.36789 | −0.4958 | 0.464972 | 0 | 0 | 0.255069 | 8 | 496.4352 | 0.792953 |
| 3006 | −0.36683 | −0.32775 | 0 | 0.247282 | 0.215748 | 0 | 8 | 502.0108 | 0.783929 |
| 3007 | −0.36769 | −0.32552 | 0 | 0.266766 | 0 | 0.223296 | 8 | 501.2214 | 0.785175 |
| 3008 | −0.35346 | −0.32601 | 0 | 0 | 0.190682 | 0.171912 | 8 | 503.0441 | 0.783419 |
| 3009 | −0.36573 | 0 | 0.297911 | 0.217151 | 0.249518 | 0 | 8 | 506.0807 | 0.780398 |
| 3010 | −0.36751 | 0 | 0.304766 | 0.234822 | 0 | 0.26618 | 8 | 504.9784 | 0.78099 |
| 3011 | −0.35702 | 0 | 0.341819 | 0 | 0.21209 | 0.213821 | 8 | 506.0056 | 0.780214 |
| 3012 | −0.36116 | 0 | 0 | 0.256551 | 0.172681 | 0.188346 | 8 | 506.7854 | 0.778315 |
| 3013 | −0.34954 | 0 | 0.311734 | 0 | 0 | 0 | 8 | 545.6677 | 0.729565 |
| 3014 | −0.34838 | −0.35299 | 0 | 0 | 0 | 0 | 8 | 554.186 | 0.712171 |
| 3015 | −0.35731 | 0 | 0 | 0.283735 | 0 | 0 | 8 | 552.3184 | 0.718214 |
| 3016 | −0.34421 | 0 | 0 | 0 | 0.260301 | 0 | 8 | 552.4651 | 0.71997 |
| 3017 | −0.34349 | 0 | 0 | 0 | 0 | 0.25144 | 8 | 552.7443 | 0.716213 |
| 3018 | −0.34954 | 0 | 0.311734 | 0 | 0 | 0 | 8 | 545.9633 | 0.728095 |
| 3019 | −0.34838 | −0.35299 | 0 | 0 | 0 | 0 | 8 | 554.153 | 0.713131 |
| 3020 | −0.35731 | 0 | 0 | 0.283735 | 0 | 0 | 8 | 552.3318 | 0.71801 |
| 3021 | −0.34421 | 0 | 0 | 0 | 0.260301 | 0 | 8 | 553.1672 | 0.721562 |
| 3022 | −0.34349 | 0 | 0 | 0 | 0 | 0.25144 | 8 | 552.7558 | 0.717479 |
| 3023 | −0.34954 | 0 | 0.311734 | 0 | 0 | 0 | 8 | 544.2172 | 0.730871 |
| 3024 | −0.34838 | −0.35299 | 0 | 0 | 0 | 0 | 8 | 551.4869 | 0.71654 |
| 3025 | −0.35731 | 0 | 0 | 0.283735 | 0 | 0 | 8 | 549.9686 | 0.721991 |
| 3026 | −0.34421 | 0 | 0 | 0 | 0.260301 | 0 | 8 | 550.4414 | 0.721603 |
| 3027 | −0.34349 | 0 | 0 | 0 | 0 | 0.25144 | 8 | 549.0226 | 0.723828 |
| 3028 | −0.3635 | 0 | 0.280897 | 0.247949 | 0 | 0 | 8 | 543.4071 | 0.731749 |
| 3029 | −0.36484 | −0.51223 | 0.448932 | 0 | 0 | 0 | 8 | 537.6207 | 0.738159 |
| 3030 | −0.36535 | −0.34122 | 0 | 0.272545 | 0 | 0 | 8 | 550.4397 | 0.717785 |
| 3031 | −0.35247 | −0.33749 | 0 | 0 | 0.244058 | 0 | 8 | 551.1145 | 0.720194 |
| 3032 | −0.3509 | −0.33426 | 0 | 0 | 0 | 0.230316 | 8 | 551.0496 | 0.716785 |
| 3033 | −0.35435 | 0 | 0.326983 | 0 | 0.276248 | 0 | 8 | 541.9639 | 0.731708 |
| 3034 | −0.35429 | 0 | 0.335115 | 0 | 0 | 0.278056 | 8 | 541.0958 | 0.734709 |
| 3035 | −0.35918 | 0 | 0 | 0.257296 | 0.231108 | 0 | 8 | 549.9542 | 0.72048 |
| 3036 | −0.36046 | 0 | 0 | 0.27483 | 0 | 0.241127 | 8 | 549.0057 | 0.720603 |
| 3037 | −0.34604 | 0 | 0 | 0 | 0.20186 | 0.189915 | 8 | 551.4834 | 0.718214 |
| 3038 | −0.34954 | 0 | 0.311734 | 0 | 0 | 0 | 8 | 545.2122 | 0.729851 |
| 3039 | −0.34838 | −0.35299 | 0 | 0 | 0 | 0 | 8 | 553.6284 | 0.71358 |
| 3040 | −0.35731 | 0 | 0 | 0.283735 | 0 | 0 | 8 | 551.6136 | 0.719541 |
| 3041 | −0.34421 | 0 | 0 | 0 | 0.260301 | 0 | 8 | 551.839 | 0.722236 |
| 3042 | −0.34349 | 0 | 0 | 0 | 0 | 0.25144 | 8 | 551.9732 | 0.718929 |
| 3043 | −0.34954 | 0 | 0.311734 | 0 | 0 | 0 | 8 | 543.8564 | 0.732464 |
| 3044 | −0.34838 | −0.35299 | 0 | 0 | 0 | 0 | 8 | 551.3706 | 0.717561 |
| 3045 | −0.35731 | 0 | 0 | 0.283735 | 0 | 0 | 8 | 549.6117 | 0.722685 |
| 3046 | −0.34421 | 0 | 0 | 0 | 0.260301 | 0 | 8 | 549.6486 | 0.722644 |
| 3047 | −0.34349 | 0 | 0 | 0 | 0 | 0.25144 | 8 | 548.6821 | 0.72489 |
| 3048 | −0.3635 | 0 | 0.280897 | 0.247949 | 0 | 0 | 8 | 542.4851 | 0.73424 |
| 3049 | −0.36484 | −0.51223 | 0.448932 | 0 | 0 | 0 | 8 | 536.7956 | 0.739364 |
| 3050 | −0.36535 | −0.34122 | 0 | 0.272545 | 0 | 0 | 8 | 549.713 | 0.720296 |
| 3051 | −0.35247 | −0.33749 | 0 | 0 | 0.244058 | 0 | 8 | 549.8271 | 0.723318 |
| 3052 | −0.3509 | −0.33426 | 0 | 0 | 0 | 0.230316 | 8 | 550.2861 | 0.718459 |
| 3053 | −0.35435 | 0 | 0.326983 | 0 | 0.276248 | 0 | 8 | 540.1206 | 0.736935 |
| 3054 | −0.35429 | 0 | 0.335115 | 0 | 0 | 0.278056 | 8 | 540.0252 | 0.736261 |
| 3055 | −0.35918 | 0 | 0 | 0.257296 | 0.231108 | 0 | 8 | 548.5104 | 0.723787 |
| 3056 | −0.36046 | 0 | 0 | 0.27483 | 0 | 0.241127 | 8 | 548.111 | 0.723195 |
| 3057 | −0.34604 | 0 | 0 | 0 | 0.20186 | 0.189915 | 8 | 550.1125 | 0.721195 |
| 3058 | −0.34954 | 0 | 0.311734 | 0 | 0 | 0 | 8 | 543.8042 | 0.732076 |
| 3059 | −0.34838 | −0.35299 | 0 | 0 | 0 | 0 | 8 | 551.0701 | 0.719255 |
| 3060 | −0.35731 | 0 | 0 | 0.283735 | 0 | 0 | 8 | 549.3045 | 0.724216 |
| 3061 | −0.34421 | 0 | 0 | 0 | 0.260301 | 0 | 8 | 549.8533 | 0.724951 |
| 3062 | −0.34349 | 0 | 0 | 0 | 0 | 0.25144 | 8 | 548.2912 | 0.725543 |
| 3063 | −0.3635 | 0 | 0.280897 | 0.247949 | 0 | 0 | 8 | 542.9061 | 0.731872 |
| 3064 | −0.36484 | −0.51223 | 0.448932 | 0 | 0 | 0 | 8 | 537.3166 | 0.737955 |
| 3065 | −0.36535 | −0.34122 | 0 | 0.272545 | 0 | 0 | 8 | 549.8308 | 0.720256 |
| 3066 | −0.35247 | −0.33749 | 0 | 0 | 0.244058 | 0 | 8 | 550.5634 | 0.721685 |
| 3067 | −0.3509 | −0.33426 | 0 | 0 | 0 | 0.230316 | 8 | 550.4124 | 0.720051 |
| 3068 | −0.35435 | 0 | 0.326983 | 0 | 0.276248 | 0 | 8 | 541.5899 | 0.733934 |
| 3069 | −0.35429 | 0 | 0.335115 | 0 | 0 | 0.278056 | 8 | 540.6138 | 0.735281 |
| 3070 | −0.35918 | 0 | 0 | 0.257296 | 0.231108 | 0 | 8 | 549.3729 | 0.721093 |
| 3071 | −0.36046 | 0 | 0 | 0.27483 | 0 | 0.241127 | 8 | 548.3736 | 0.721501 |
| 3072 | −0.34604 | 0 | 0 | 0 | 0.20186 | 0.189915 | 8 | 550.8698 | 0.721113 |
| 3073 | −0.3635 | 0 | 0.280897 | 0.247949 | 0 | 0 | 8 | 540.8855 | 0.736669 |
| 3074 | −0.36484 | −0.51223 | 0.448932 | 0 | 0 | 0 | 8 | 535.0127 | 0.74214 |
| 3075 | −0.36535 | −0.34122 | 0 | 0.272545 | 0 | 0 | 8 | 546.9117 | 0.723665 |
| 3076 | −0.35247 | −0.33749 | 0 | 0 | 0.244058 | 0 | 8 | 547.3152 | 0.724992 |
| 3077 | −0.3509 | −0.33426 | 0 | 0 | 0 | 0.230316 | 8 | 546.1642 | 0.726013 |
| 3078 | −0.35435 | 0 | 0.326983 | 0 | 0.276248 | 0 | 8 | 539.067 | 0.736322 |
| 3079 | −0.35429 | 0 | 0.335115 | 0 | 0 | 0.278056 | 8 | 536.847 | 0.741426 |
| 3080 | −0.35918 | 0 | 0 | 0.257296 | 0.231108 | 0 | 8 | 546.3241 | 0.726217 |
| 3081 | −0.36046 | 0 | 0 | 0.27483 | 0 | 0.241127 | 8 | 544.1375 | 0.730708 |
| 3082 | −0.34604 | 0 | 0 | 0 | 0.20186 | 0.189915 | 8 | 546.8609 | 0.727197 |
| 3083 | −0.37665 | −0.49016 | 0.415897 | 0.213769 | 0 | 0 | 8 | 535.4324 | 0.739915 |
| 3084 | −0.3692 | −0.49878 | 0.458935 | 0 | 0.257995 | 0 | 8 | 533.5052 | 0.742059 |
| 3085 | −0.36789 | −0.4958 | 0.464972 | 0 | 0 | 0.255069 | 8 | 533.0413 | 0.74459 |
| 3086 | −0.36683 | −0.32775 | 0 | 0.247282 | 0.215748 | 0 | 8 | 547.6623 | 0.722869 |
| 3087 | −0.36769 | −0.32552 | 0 | 0.266766 | 0 | 0.223296 | 8 | 546.9649 | 0.721521 |
| 3088 | −0.35346 | −0.32601 | 0 | 0 | 0.190682 | 0.171912 | 8 | 549.2107 | 0.720154 |
| 3089 | −0.36573 | 0 | 0.297911 | 0.217151 | 0.249518 | 0 | 8 | 539.8173 | 0.735424 |
| 3090 | −0.36751 | 0 | 0.304766 | 0.234822 | 0 | 0.26618 | 8 | 538.5215 | 0.737772 |
| 3091 | −0.35702 | 0 | 0.341819 | 0 | 0.21209 | 0.213821 | 8 | 539.0304 | 0.736751 |
| 3092 | −0.36116 | 0 | 0 | 0.256551 | 0.172681 | 0.188346 | 8 | 547.8354 | 0.722154 |
| 3093 | −0.34954 | 0 | 0.311734 | 0 | 0 | 0 | 8 | 546.281 | 0.728279 |
| 3094 | −0.34838 | −0.35299 | 0 | 0 | 0 | 0 | 8 | 554.1339 | 0.713621 |
| 3095 | −0.35731 | 0 | 0 | 0.283735 | 0 | 0 | 8 | 552.1716 | 0.719276 |
| 3096 | −0.34421 | 0 | 0 | 0 | 0.260301 | 0 | 8 | 552.5699 | 0.720419 |
| 3097 | −0.34349 | 0 | 0 | 0 | 0 | 0.25144 | 8 | 552.5309 | 0.716846 |
| 3098 | −0.34954 | 0 | 0.311734 | 0 | 0 | 0 | 8 | 544.7883 | 0.730749 |
| 3099 | −0.34838 | −0.35299 | 0 | 0 | 0 | 0 | 8 | 551.7237 | 0.71554 |
| 3100 | −0.35731 | 0 | 0 | 0.283735 | 0 | 0 | 8 | 550.0825 | 0.722848 |
| 3101 | −0.34421 | 0 | 0 | 0 | 0.260301 | 0 | 8 | 550.2738 | 0.720848 |
| 3102 | −0.34349 | 0 | 0 | 0 | 0 | 0.25144 | 8 | 549.2057 | 0.724257 |
| 3103 | −0.3635 | 0 | 0.280897 | 0.247949 | 0 | 0 | 8 | 543.3014 | 0.7323 |
| 3104 | −0.36484 | −0.51223 | 0.448932 | 0 | 0 | 0 | 8 | 537.8443 | 0.737261 |
| 3105 | −0.36535 | −0.34122 | 0 | 0.272545 | 0 | 0 | 8 | 550.0853 | 0.719929 |
| 3106 | −0.35247 | −0.33749 | 0 | 0 | 0.244058 | 0 | 8 | 550.3638 | 0.722481 |
| 3107 | −0.3509 | −0.33426 | 0 | 0 | 0 | 0.230316 | 8 | 550.6611 | 0.717275 |
| 3108 | −0.35435 | 0 | 0.326983 | 0 | 0.276248 | 0 | 8 | 541.314 | 0.735016 |
| 3109 | −0.35429 | 0 | 0.335115 | 0 | 0 | 0.278056 | 8 | 540.9115 | 0.734485 |
| 3110 | −0.35918 | 0 | 0 | 0.257296 | 0.231108 | 0 | 8 | 549.027 | 0.722889 |
| 3111 | −0.36046 | 0 | 0 | 0.27483 | 0 | 0.241127 | 8 | 548.4084 | 0.722501 |
| 3112 | −0.34604 | 0 | 0 | 0 | 0.20186 | 0.189915 | 8 | 550.6112 | 0.720705 |
| 3113 | −0.34954 | 0 | 0.311734 | 0 | 0 | 0 | 8 | 545.0705 | 0.729381 |
| 3114 | −0.34838 | −0.35299 | 0 | 0 | 0 | 0 | 8 | 551.6722 | 0.716213 |
| 3115 | −0.35731 | 0 | 0 | 0.283735 | 0 | 0 | 8 | 550.0448 | 0.72344 |
| 3116 | −0.34421 | 0 | 0 | 0 | 0.260301 | 0 | 8 | 550.8411 | 0.721746 |
| 3117 | −0.34349 | 0 | 0 | 0 | 0 | 0.25144 | 8 | 549.1088 | 0.723685 |
| 3118 | −0.3635 | 0 | 0.280897 | 0.247949 | 0 | 0 | 8 | 543.9984 | 0.730463 |
| 3119 | −0.36484 | −0.51223 | 0.448932 | 0 | 0 | 0 | 8 | 538.693 | 0.735036 |
| 3120 | −0.36535 | −0.34122 | 0 | 0.272545 | 0 | 0 | 8 | 550.4205 | 0.718908 |
| 3121 | −0.35247 | −0.33749 | 0 | 0 | 0.244058 | 0 | 8 | 551.3633 | 0.719949 |
| 3122 | −0.3509 | −0.33426 | 0 | 0 | 0 | 0.230316 | 8 | 550.9984 | 0.717336 |
| 3123 | −0.35435 | 0 | 0.326983 | 0 | 0.276248 | 0 | 8 | 543.0829 | 0.730953 |
| 3124 | −0.35429 | 0 | 0.335115 | 0 | 0 | 0.278056 | 8 | 541.76 | 0.733199 |
| 3125 | −0.35918 | 0 | 0 | 0.257296 | 0.231108 | 0 | 8 | 550.1119 | 0.720256 |
| 3126 | −0.36046 | 0 | 0 | 0.27483 | 0 | 0.241127 | 8 | 548.8457 | 0.721236 |
| 3127 | −0.34604 | 0 | 0 | 0 | 0.20186 | 0.189915 | 8 | 551.5833 | 0.718214 |
| 3128 | −0.3635 | 0 | 0.280897 | 0.247949 | 0 | 0 | 8 | 541.9337 | 0.734689 |
| 3129 | −0.36484 | −0.51223 | 0.448932 | 0 | 0 | 0 | 8 | 536.2962 | 0.738343 |
| 3130 | −0.36535 | −0.34122 | 0 | 0.272545 | 0 | 0 | 8 | 547.4557 | 0.723318 |
| 3131 | −0.35247 | −0.33749 | 0 | 0 | 0.244058 | 0 | 8 | 548.0935 | 0.723849 |
| 3132 | −0.3509 | −0.33426 | 0 | 0 | 0 | 0.230316 | 8 | 546.7725 | 0.723644 |
| 3133 | −0.35435 | 0 | 0.326983 | 0 | 0.276248 | 0 | 8 | 540.6004 | 0.733117 |
| 3134 | −0.35429 | 0 | 0.335115 | 0 | 0 | 0.278056 | 8 | 538.1151 | 0.738751 |
| 3135 | −0.35918 | 0 | 0 | 0.257296 | 0.231108 | 0 | 8 | 547.1257 | 0.724665 |
| 3136 | −0.36046 | 0 | 0 | 0.27483 | 0 | 0.241127 | 8 | 544.7671 | 0.729871 |
| 3137 | −0.34604 | 0 | 0 | 0 | 0.20186 | 0.189915 | 8 | 547.6969 | 0.724665 |
| 3138 | −0.37665 | −0.49016 | 0.415897 | 0.213769 | 0 | 0 | 8 | 536.5217 | 0.738731 |
| 3139 | −0.3692 | −0.49878 | 0.458935 | 0 | 0.257995 | 0 | 8 | 535.0224 | 0.739752 |
| 3140 | −0.36789 | −0.4958 | 0.464972 | 0 | 0 | 0.255069 | 8 | 534.1849 | 0.742814 |
| 3141 | −0.36683 | −0.32775 | 0 | 0.247282 | 0.215748 | 0 | 8 | 548.1536 | 0.723195 |
| 3142 | −0.36769 | −0.32552 | 0 | 0.266766 | 0 | 0.223296 | 8 | 547.2108 | 0.72197 |
| 3143 | −0.35346 | −0.32601 | 0 | 0 | 0.190682 | 0.171912 | 8 | 549.6944 | 0.72048 |
| 3144 | −0.36573 | 0 | 0.297911 | 0.217151 | 0.249518 | 0 | 8 | 540.8686 | 0.733403 |
| 3145 | −0.36751 | 0 | 0.304766 | 0.234822 | 0 | 0.26618 | 8 | 539.1692 | 0.736792 |
| 3146 | −0.35702 | 0 | 0.341819 | 0 | 0.21209 | 0.213821 | 8 | 540.0995 | 0.733852 |
| 3147 | −0.36116 | 0 | 0 | 0.256551 | 0.172681 | 0.188346 | 8 | 548.112 | 0.722848 |
| 3148 | −0.34954 | 0 | 0.311734 | 0 | 0 | 0 | 8 | 544.7634 | 0.731749 |
| 3149 | −0.34838 | −0.35299 | 0 | 0 | 0 | 0 | 8 | 551.6523 | 0.717806 |
| 3150 | −0.35731 | 0 | 0 | 0.283735 | 0 | 0 | 8 | 549.72 | 0.723992 |
| 3151 | −0.34421 | 0 | 0 | 0 | 0.260301 | 0 | 8 | 550.0675 | 0.722011 |
| 3152 | −0.34349 | 0 | 0 | 0 | 0 | 0.25144 | 8 | 548.7969 | 0.724726 |
| 3153 | −0.3635 | 0 | 0.280897 | 0.247949 | 0 | 0 | 8 | 542.9928 | 0.732729 |
| 3154 | −0.36484 | −0.51223 | 0.448932 | 0 | 0 | 0 | 8 | 537.8432 | 0.737343 |
| 3155 | −0.36535 | −0.34122 | 0 | 0.272545 | 0 | 0 | 8 | 549.6496 | 0.720684 |
| 3156 | −0.35247 | −0.33749 | 0 | 0 | 0.244058 | 0 | 8 | 550.0123 | 0.723706 |
| 3157 | −0.3509 | −0.33426 | 0 | 0 | 0 | 0.230316 | 8 | 550.1637 | 0.718704 |
| 3158 | −0.35435 | 0 | 0.326983 | 0 | 0.276248 | 0 | 8 | 541.128 | 0.734566 |
| 3159 | −0.35429 | 0 | 0.335115 | 0 | 0 | 0.278056 | 8 | 540.5539 | 0.735832 |
| 3160 | −0.35918 | 0 | 0 | 0.257296 | 0.231108 | 0 | 8 | 548.5456 | 0.723399 |
| 3161 | −0.36046 | 0 | 0 | 0.27483 | 0 | 0.241127 | 8 | 547.8052 | 0.723195 |
| 3162 | −0.34604 | 0 | 0 | 0 | 0.20186 | 0.189915 | 8 | 550.0825 | 0.721705 |
| 3163 | −0.3635 | 0 | 0.280897 | 0.247949 | 0 | 0 | 8 | 541.3865 | 0.735036 |
| 3164 | −0.36484 | −0.51223 | 0.448932 | 0 | 0 | 0 | 8 | 536.0298 | 0.740446 |
| 3165 | −0.36535 | −0.34122 | 0 | 0.272545 | 0 | 0 | 8 | 547.1508 | 0.725339 |
| 3166 | −0.35247 | −0.33749 | 0 | 0 | 0.244058 | 0 | 8 | 547.3998 | 0.725808 |
| 3167 | −0.3509 | −0.33426 | 0 | 0 | 0 | 0.230316 | 8 | 546.5005 | 0.725298 |
| 3168 | −0.35435 | 0 | 0.326983 | 0 | 0.276248 | 0 | 8 | 539.3415 | 0.736057 |
| 3169 | −0.35429 | 0 | 0.335115 | 0 | 0 | 0.278056 | 8 | 537.5222 | 0.740507 |
| 3170 | −0.35918 | 0 | 0 | 0.257296 | 0.231108 | 0 | 8 | 546.1507 | 0.727421 |
| 3171 | −0.36046 | 0 | 0 | 0.27483 | 0 | 0.241127 | 8 | 544.2273 | 0.730892 |
| 3172 | −0.34604 | 0 | 0 | 0 | 0.20186 | 0.189915 | 8 | 546.8409 | 0.726482 |
| 3173 | −0.37665 | −0.49016 | 0.415897 | 0.213769 | 0 | 0 | 8 | 535.4363 | 0.740834 |
| 3174 | −0.3692 | −0.49878 | 0.458935 | 0 | 0.257995 | 0 | 8 | 532.9921 | 0.744631 |
| 3175 | −0.36789 | −0.4958 | 0.464972 | 0 | 0 | 0.255069 | 8 | 532.901 | 0.744243 |
| 3176 | −0.36683 | −0.32775 | 0 | 0.247282 | 0.215748 | 0 | 8 | 546.6424 | 0.727319 |
| 3177 | −0.36769 | −0.32552 | 0 | 0.266766 | 0 | 0.223296 | 8 | 546.1974 | 0.724584 |
| 3178 | −0.35346 | −0.32601 | 0 | 0 | 0.190682 | 0.171912 | 8 | 548.2625 | 0.723481 |
| 3179 | −0.36573 | 0 | 0.297911 | 0.217151 | 0.249518 | 0 | 8 | 538.835 | 0.738568 |
| 3180 | −0.36751 | 0 | 0.304766 | 0.234822 | 0 | 0.26618 | 8 | 537.8502 | 0.740323 |
| 3181 | −0.35702 | 0 | 0.341819 | 0 | 0.21209 | 0.213821 | 8 | 538.0549 | 0.73916 |
| 3182 | −0.36116 | 0 | 0 | 0.256551 | 0.172681 | 0.188346 | 8 | 546.5357 | 0.72587 |
| 3183 | −0.3635 | 0 | 0.280897 | 0.247949 | 0 | 0 | 8 | 541.6787 | 0.734342 |
| 3184 | −0.36484 | −0.51223 | 0.448932 | 0 | 0 | 0 | 8 | 536.4116 | 0.738568 |
| 3185 | −0.36535 | −0.34122 | 0 | 0.272545 | 0 | 0 | 8 | 547.1596 | 0.724971 |
| 3186 | −0.35247 | −0.33749 | 0 | 0 | 0.244058 | 0 | 8 | 547.8813 | 0.72587 |
| 3187 | −0.3509 | −0.33426 | 0 | 0 | 0 | 0.230316 | 8 | 546.4516 | 0.726319 |
| 3188 | −0.35435 | 0 | 0.326983 | 0 | 0.276248 | 0 | 8 | 540.4426 | 0.733525 |
| 3189 | −0.35429 | 0 | 0.335115 | 0 | 0 | 0.278056 | 8 | 537.8292 | 0.74018 |
| 3190 | −0.35918 | 0 | 0 | 0.257296 | 0.231108 | 0 | 8 | 546.7074 | 0.725931 |
| 3191 | −0.36046 | 0 | 0 | 0.27483 | 0 | 0.241127 | 8 | 544.2101 | 0.730606 |
| 3192 | −0.34604 | 0 | 0 | 0 | 0.20186 | 0.189915 | 8 | 547.259 | 0.726013 |
| 3193 | −0.37665 | −0.49016 | 0.415897 | 0.213769 | 0 | 0 | 8 | 536.3444 | 0.738792 |
| 3194 | −0.3692 | −0.49878 | 0.458935 | 0 | 0.257995 | 0 | 8 | 534.9019 | 0.74016 |
| 3195 | −0.36789 | −0.4958 | 0.464972 | 0 | 0 | 0.255069 | 8 | 533.996 | 0.742916 |
| 3196 | −0.36683 | −0.32775 | 0 | 0.247282 | 0.215748 | 0 | 8 | 547.7519 | 0.723665 |
| 3197 | −0.36769 | −0.32552 | 0 | 0.266766 | 0 | 0.223296 | 8 | 546.7546 | 0.722134 |
| 3198 | −0.35346 | −0.32601 | 0 | 0 | 0.190682 | 0.171912 | 8 | 549.269 | 0.720929 |
| 3199 | −0.36573 | 0 | 0.297911 | 0.217151 | 0.249518 | 0 | 8 | 540.6458 | 0.733995 |
| 3200 | −0.36751 | 0 | 0.304766 | 0.234822 | 0 | 0.26618 | 8 | 538.8968 | 0.736363 |
| 3201 | −0.35702 | 0 | 0.341819 | 0 | 0.21209 | 0.213821 | 8 | 539.8787 | 0.735118 |
| 3202 | −0.36116 | 0 | 0 | 0.256551 | 0.172681 | 0.188346 | 8 | 547.6997 | 0.721664 |
| 3203 | −0.37665 | −0.49016 | 0.415897 | 0.213769 | 0 | 0 | 8 | 533.8465 | 0.743814 |
| 3204 | −0.3692 | −0.49878 | 0.458935 | 0 | 0.257995 | 0 | 8 | 531.9778 | 0.744978 |
| 3205 | −0.36789 | −0.4958 | 0.464972 | 0 | 0 | 0.255069 | 8 | 529.8993 | 0.74851 |
| 3206 | −0.36683 | −0.32775 | 0 | 0.247282 | 0.215748 | 0 | 8 | 544.2777 | 0.728564 |
| 3207 | −0.36769 | −0.32552 | 0 | 0.266766 | 0 | 0.223296 | 8 | 542.1378 | 0.73181 |
| 3208 | −0.35346 | −0.32601 | 0 | 0 | 0.190682 | 0.171912 | 8 | 544.9504 | 0.72836 |
| 3209 | −0.36573 | 0 | 0.297911 | 0.217151 | 0.249518 | 0 | 8 | 537.9362 | 0.736935 |
| 3210 | −0.36751 | 0 | 0.304766 | 0.234822 | 0 | 0.26618 | 8 | 534.8902 | 0.743835 |
| 3211 | −0.35702 | 0 | 0.341819 | 0 | 0.21209 | 0.213821 | 8 | 536.0159 | 0.740507 |
| 3212 | −0.36116 | 0 | 0 | 0.256551 | 0.172681 | 0.188346 | 8 | 543.3721 | 0.72981 |
| 3213 | −0.3784 | −0.48038 | 0.429094 | 0.184036 | 0.235336 | 0 | 8 | 533.3249 | 0.743508 |
| 3214 | −0.37944 | −0.47686 | 0.432955 | 0.204998 | 0 | 0.247828 | 8 | 532.0386 | 0.745754 |
| 3215 | −0.37057 | −0.48826 | 0.469023 | 0 | 0.198485 | 0.194003 | 8 | 532.3611 | 0.744488 |
| 3216 | −0.36821 | −0.31823 | 0 | 0.248938 | 0.16128 | 0.17389 | 8 | 546.0943 | 0.723481 |
| 3217 | −0.36844 | 0 | 0.31303 | 0.214129 | 0.186465 | 0.210249 | 8 | 537.8308 | 0.738609 |
| 3218 | −0.3635 | 0 | 0.280897 | 0.247949 | 0 | 0 | 8 | 574.4132 | 0.676425 |
| 3219 | −0.36484 | −0.51223 | 0.448932 | 0 | 0 | 0 | 8 | 563.3614 | 0.697024 |
| 3220 | −0.36535 | −0.34122 | 0 | 0.272545 | 0 | 0 | 8 | 572.2047 | 0.683897 |
| 3221 | −0.35247 | −0.33749 | 0 | 0 | 0.244058 | 0 | 8 | 572.0823 | 0.68553 |
| 3222 | −0.3509 | −0.33426 | 0 | 0 | 0 | 0.230316 | 8 | 571.6508 | 0.682651 |
| 3223 | −0.35435 | 0 | 0.326983 | 0 | 0.276248 | 0 | 8 | 571.3319 | 0.67865 |
| 3224 | −0.35429 | 0 | 0.335115 | 0 | 0 | 0.278056 | 8 | 570.2574 | 0.683713 |
| 3225 | −0.35918 | 0 | 0 | 0.257296 | 0.231108 | 0 | 8 | 574.4302 | 0.676384 |
| 3226 | −0.36046 | 0 | 0 | 0.27483 | 0 | 0.241127 | 8 | 572.7513 | 0.677466 |
| 3227 | −0.34604 | 0 | 0 | 0 | 0.20186 | 0.189915 | 8 | 575.5865 | 0.674608 |
| 3228 | −0.37665 | −0.49016 | 0.415897 | 0.213769 | 0 | 0 | 8 | 561.6475 | 0.697983 |
| 3229 | −0.3692 | −0.49878 | 0.458935 | 0 | 0.257995 | 0 | 8 | 558.3771 | 0.70472 |
| 3230 | −0.36789 | −0.4958 | 0.464972 | 0 | 0 | 0.255069 | 8 | 559.2688 | 0.698779 |
| 3231 | −0.36683 | −0.32775 | 0 | 0.247282 | 0.215748 | 0 | 8 | 570.1492 | 0.688163 |
| 3232 | −0.36769 | −0.32552 | 0 | 0.266766 | 0 | 0.223296 | 8 | 570.2574 | 0.681406 |
| 3233 | −0.35346 | −0.32601 | 0 | 0 | 0.190682 | 0.171912 | 8 | 572.7142 | 0.682284 |
| 3234 | −0.36573 | 0 | 0.297911 | 0.217151 | 0.249518 | 0 | 8 | 569.6765 | 0.685591 |
| 3235 | −0.36751 | 0 | 0.304766 | 0.234822 | 0 | 0.26618 | 8 | 569.5213 | 0.684081 |
| 3236 | −0.35702 | 0 | 0.341819 | 0 | 0.21209 | 0.213821 | 8 | 569.6513 | 0.684407 |
| 3237 | −0.36116 | 0 | 0 | 0.256551 | 0.172681 | 0.188346 | 8 | 574.3286 | 0.675016 |
| 3238 | −0.37665 | −0.49016 | 0.415897 | 0.213769 | 0 | 0 | 8 | 559.4448 | 0.70572 |
| 3239 | −0.3692 | −0.49878 | 0.458935 | 0 | 0.257995 | 0 | 8 | 555.9413 | 0.708762 |
| 3240 | −0.36789 | −0.4958 | 0.464972 | 0 | 0 | 0.255069 | 8 | 555.1873 | 0.706966 |
| 3241 | −0.36683 | −0.32775 | 0 | 0.247282 | 0.215748 | 0 | 8 | 567.0218 | 0.693349 |
| 3242 | −0.36769 | −0.32552 | 0 | 0.266766 | 0 | 0.223296 | 8 | 565.559 | 0.691614 |
| 3243 | −0.35346 | −0.32601 | 0 | 0 | 0.190682 | 0.171912 | 8 | 568.4371 | 0.688633 |
| 3244 | −0.36573 | 0 | 0.297911 | 0.217151 | 0.249518 | 0 | 8 | 567.5331 | 0.687306 |
| 3245 | −0.36751 | 0 | 0.304766 | 0.234822 | 0 | 0.26618 | 8 | 565.6642 | 0.691756 |
| 3246 | −0.35702 | 0 | 0.341819 | 0 | 0.21209 | 0.213821 | 8 | 566.0561 | 0.689674 |
| 3247 | −0.36116 | 0 | 0 | 0.256551 | 0.172681 | 0.188346 | 8 | 570.172 | 0.680794 |
| 3248 | −0.3784 | −0.48038 | 0.429094 | 0.184036 | 0.235336 | 0 | 8 | 555.7991 | 0.707558 |
| 3249 | −0.37944 | −0.47686 | 0.432955 | 0.204998 | 0 | 0.247828 | 8 | 555.9535 | 0.704985 |
| 3250 | −0.37057 | −0.48826 | 0.469023 | 0 | 0.198485 | 0.194003 | 8 | 555.3793 | 0.704965 |
| 3251 | −0.36821 | −0.31823 | 0 | 0.248938 | 0.16128 | 0.17389 | 8 | 568.0284 | 0.687143 |
| 3252 | −0.36844 | 0 | 0.31303 | 0.214129 | 0.186465 | 0.210249 | 8 | 566.2489 | 0.689633 |
| 3253 | −0.37665 | −0.49016 | 0.415897 | 0.213769 | 0 | 0 | 8 | 559.5021 | 0.705945 |
| 3254 | −0.3692 | −0.49878 | 0.458935 | 0 | 0.257995 | 0 | 8 | 556.576 | 0.706823 |
| 3255 | −0.36789 | −0.4958 | 0.464972 | 0 | 0 | 0.255069 | 8 | 555.2393 | 0.707476 |
| 3256 | −0.36683 | −0.32775 | 0 | 0.247282 | 0.215748 | 0 | 8 | 567.2179 | 0.694329 |
| 3257 | −0.36769 | −0.32552 | 0 | 0.266766 | 0 | 0.223296 | 8 | 565.3007 | 0.693553 |
| 3258 | −0.35346 | −0.32601 | 0 | 0 | 0.190682 | 0.171912 | 8 | 568.47 | 0.690736 |
| 3259 | −0.36573 | 0 | 0.297911 | 0.217151 | 0.249518 | 0 | 8 | 567.955 | 0.68506 |
| 3260 | −0.36751 | 0 | 0.304766 | 0.234822 | 0 | 0.26618 | 8 | 565.5113 | 0.690062 |
| 3261 | −0.35702 | 0 | 0.341819 | 0 | 0.21209 | 0.213821 | 8 | 566.3544 | 0.687816 |
| 3262 | −0.36116 | 0 | 0 | 0.256551 | 0.172681 | 0.188346 | 8 | 570.1845 | 0.68063 |
| 3263 | −0.3784 | −0.48038 | 0.429094 | 0.184036 | 0.235336 | 0 | 8 | 557.0221 | 0.705496 |
| 3264 | −0.37944 | −0.47686 | 0.432955 | 0.204998 | 0 | 0.247828 | 8 | 556.5276 | 0.702678 |
| 3265 | −0.37057 | −0.48826 | 0.469023 | 0 | 0.198485 | 0.194003 | 8 | 556.565 | 0.702413 |
| 3266 | −0.36821 | −0.31823 | 0 | 0.248938 | 0.16128 | 0.17389 | 8 | 568.613 | 0.686347 |
| 3267 | −0.36844 | 0 | 0.31303 | 0.214129 | 0.186465 | 0.210249 | 8 | 567.1915 | 0.686837 |
| 3268 | −0.3784 | −0.48038 | 0.429094 | 0.184036 | 0.235336 | 0 | 8 | 553.8625 | 0.711049 |
| 3269 | −0.37944 | −0.47686 | 0.432955 | 0.204998 | 0 | 0.247828 | 8 | 551.6937 | 0.713968 |
| 3270 | −0.37057 | −0.48826 | 0.469023 | 0 | 0.198485 | 0.194003 | 8 | 551.9132 | 0.71013 |
| 3271 | −0.36821 | −0.31823 | 0 | 0.248938 | 0.16128 | 0.17389 | 8 | 563.393 | 0.696044 |
| 3272 | −0.36844 | 0 | 0.31303 | 0.214129 | 0.186465 | 0.210249 | 8 | 562.7233 | 0.693614 |
| 3273 | 0 | 0 | 0 | 0 | 0 | 0 | 8 | 512.7521 | 0.777866 |
| 3274 | −0.38017 | −0.47149 | 0.43946 | 0.184707 | 0.175726 | 0.194494 | 8 | 554.1601 | 0.705271 |
| 3275 | −0.34954 | 0 | 0.311734 | 0 | 0 | 0 | 7 | 548.4699 | 0.719949 |
| 3276 | −0.34838 | −0.35299 | 0 | 0 | 0 | 0 | 7 | 536.9434 | 0.742671 |
| 3277 | −0.35731 | 0 | 0 | 0.283735 | 0 | 0 | 7 | 545.9775 | 0.728422 |
| 3278 | −0.34421 | 0 | 0 | 0 | 0.260301 | 0 | 7 | 544.7628 | 0.727666 |
| 3279 | −0.34349 | 0 | 0 | 0 | 0 | 0.25144 | 7 | 545.9872 | 0.727319 |
| 3280 | −0.34954 | 0 | 0.311734 | 0 | 0 | 0 | 7 | 548.4014 | 0.720541 |
| 3281 | −0.34838 | −0.35299 | 0 | 0 | 0 | 0 | 7 | 536.8512 | 0.741895 |
| 3282 | −0.35731 | 0 | 0 | 0.283735 | 0 | 0 | 7 | 545.6465 | 0.728789 |
| 3283 | −0.34421 | 0 | 0 | 0 | 0.260301 | 0 | 7 | 543.9422 | 0.730096 |
| 3284 | −0.34349 | 0 | 0 | 0 | 0 | 0.25144 | 7 | 545.5631 | 0.728748 |
| 3285 | −0.34954 | 0 | 0.311734 | 0 | 0 | 0 | 7 | 547.7785 | 0.721746 |
| 3286 | −0.34838 | −0.35299 | 0 | 0 | 0 | 0 | 7 | 536.2877 | 0.742487 |
| 3287 | −0.35731 | 0 | 0 | 0.283735 | 0 | 0 | 7 | 545.081 | 0.728381 |
| 3288 | −0.34421 | 0 | 0 | 0 | 0.260301 | 0 | 7 | 543.8204 | 0.726972 |
| 3289 | −0.34349 | 0 | 0 | 0 | 0 | 0.25144 | 7 | 544.8627 | 0.728544 |
| 3290 | −0.34954 | 0 | 0.311734 | 0 | 0 | 0 | 7 | 545.1451 | 0.724318 |
| 3291 | −0.34838 | −0.35299 | 0 | 0 | 0 | 0 | 7 | 532.0374 | 0.749388 |
| 3292 | −0.35731 | 0 | 0 | 0.283735 | 0 | 0 | 7 | 541.6002 | 0.733852 |
| 3293 | −0.34421 | 0 | 0 | 0 | 0.260301 | 0 | 7 | 539.9267 | 0.735281 |
| 3294 | −0.34349 | 0 | 0 | 0 | 0 | 0.25144 | 7 | 539.5326 | 0.737282 |
| 3295 | −0.3635 | 0 | 0.280897 | 0.247949 | 0 | 0 | 7 | 544.8216 | 0.727605 |
| 3296 | −0.36484 | −0.51223 | 0.448932 | 0 | 0 | 0 | 7 | 529.1433 | 0.750449 |
| 3297 | −0.36535 | −0.34122 | 0 | 0.272545 | 0 | 0 | 7 | 532.859 | 0.749285 |
| 3298 | −0.35247 | −0.33749 | 0 | 0 | 0.244058 | 0 | 7 | 532.0654 | 0.74753 |
| 3299 | −0.3509 | −0.33426 | 0 | 0 | 0 | 0.230316 | 7 | 533.4235 | 0.746325 |
| 3300 | −0.35435 | 0 | 0.326983 | 0 | 0.276248 | 0 | 7 | 542.2109 | 0.730157 |
| 3301 | −0.35429 | 0 | 0.335115 | 0 | 0 | 0.278056 | 7 | 543.4058 | 0.729259 |
| 3302 | −0.35918 | 0 | 0 | 0.257296 | 0.231108 | 0 | 7 | 541.1378 | 0.733484 |
| 3303 | −0.36046 | 0 | 0 | 0.27483 | 0 | 0.241127 | 7 | 541.5796 | 0.735179 |
| 3304 | −0.34604 | 0 | 0 | 0 | 0.20186 | 0.189915 | 7 | 542.5384 | 0.73179 |
| 3305 | −0.34954 | 0 | 0.311734 | 0 | 0 | 0 | 7 | 552.275 | 0.713049 |
| 3306 | −0.34838 | −0.35299 | 0 | 0 | 0 | 0 | 7 | 540.5814 | 0.735526 |
| 3307 | −0.35731 | 0 | 0 | 0.283735 | 0 | 0 | 7 | 548.9564 | 0.721583 |
| 3308 | −0.34421 | 0 | 0 | 0 | 0.260301 | 0 | 7 | 547.7822 | 0.722154 |
| 3309 | −0.34349 | 0 | 0 | 0 | 0 | 0.25144 | 7 | 549.0825 | 0.719112 |
| 3310 | −0.34954 | 0 | 0.311734 | 0 | 0 | 0 | 7 | 552.3786 | 0.710436 |
| 3311 | −0.34838 | −0.35299 | 0 | 0 | 0 | 0 | 7 | 540.7159 | 0.734811 |
| 3312 | −0.35731 | 0 | 0 | 0.283735 | 0 | 0 | 7 | 549.0725 | 0.719602 |
| 3313 | −0.34421 | 0 | 0 | 0 | 0.260301 | 0 | 7 | 548.5238 | 0.717663 |
| 3314 | −0.34349 | 0 | 0 | 0 | 0 | 0.25144 | 7 | 549.1576 | 0.717438 |
| 3315 | −0.34954 | 0 | 0.311734 | 0 | 0 | 0 | 7 | 549.1986 | 0.717683 |
| 3316 | −0.34838 | −0.35299 | 0 | 0 | 0 | 0 | 7 | 536.1593 | 0.742998 |
| 3317 | −0.35731 | 0 | 0 | 0.283735 | 0 | 0 | 7 | 545.2088 | 0.727932 |
| 3318 | −0.34421 | 0 | 0 | 0 | 0.260301 | 0 | 7 | 544.1914 | 0.727217 |
| 3319 | −0.34349 | 0 | 0 | 0 | 0 | 0.25144 | 7 | 543.5214 | 0.728013 |
| 3320 | −0.3635 | 0 | 0.280897 | 0.247949 | 0 | 0 | 7 | 548.8698 | 0.718969 |
| 3321 | −0.36484 | −0.51223 | 0.448932 | 0 | 0 | 0 | 7 | 534.158 | 0.743467 |
| 3322 | −0.36535 | −0.34122 | 0 | 0.272545 | 0 | 0 | 7 | 536.7481 | 0.741895 |
| 3323 | −0.35247 | −0.33749 | 0 | 0 | 0.244058 | 0 | 7 | 536.6376 | 0.738568 |
| 3324 | −0.3509 | −0.33426 | 0 | 0 | 0 | 0.230316 | 7 | 537.5216 | 0.739813 |
| 3325 | −0.35435 | 0 | 0.326983 | 0 | 0.276248 | 0 | 7 | 547.1327 | 0.719337 |
| 3326 | −0.35429 | 0 | 0.335115 | 0 | 0 | 0.278056 | 7 | 547.8104 | 0.720256 |
| 3327 | −0.35918 | 0 | 0 | 0.257296 | 0.231108 | 0 | 7 | 545.1626 | 0.72538 |
| 3328 | −0.36046 | 0 | 0 | 0.27483 | 0 | 0.241127 | 7 | 545.1975 | 0.727605 |
| 3329 | −0.34604 | 0 | 0 | 0 | 0.20186 | 0.189915 | 7 | 546.718 | 0.722379 |
| 3330 | −0.34954 | 0 | 0.311734 | 0 | 0 | 0 | 7 | 552.68 | 0.710456 |
| 3331 | −0.34838 | −0.35299 | 0 | 0 | 0 | 0 | 7 | 540.9732 | 0.733913 |
| 3332 | −0.35731 | 0 | 0 | 0.283735 | 0 | 0 | 7 | 549.0264 | 0.720051 |
| 3333 | −0.34421 | 0 | 0 | 0 | 0.260301 | 0 | 7 | 547.9958 | 0.720256 |
| 3334 | −0.34349 | 0 | 0 | 0 | 0 | 0.25144 | 7 | 548.9736 | 0.718561 |
| 3335 | −0.34954 | 0 | 0.311734 | 0 | 0 | 0 | 7 | 550.2639 | 0.716152 |
| 3336 | −0.34838 | −0.35299 | 0 | 0 | 0 | 0 | 7 | 537.3385 | 0.740528 |
| 3337 | −0.35731 | 0 | 0 | 0.283735 | 0 | 0 | 7 | 545.93 | 0.72689 |
| 3338 | −0.34421 | 0 | 0 | 0 | 0.260301 | 0 | 7 | 544.7535 | 0.726074 |
| 3339 | −0.34349 | 0 | 0 | 0 | 0 | 0.25144 | 7 | 544.3459 | 0.727707 |
| 3340 | −0.3635 | 0 | 0.280897 | 0.247949 | 0 | 0 | 7 | 548.6875 | 0.720786 |
| 3341 | −0.36484 | −0.51223 | 0.448932 | 0 | 0 | 0 | 7 | 534.1917 | 0.7431 |
| 3342 | −0.36535 | −0.34122 | 0 | 0.272545 | 0 | 0 | 7 | 536.4465 | 0.742794 |
| 3343 | −0.35247 | −0.33749 | 0 | 0 | 0.244058 | 0 | 7 | 535.7897 | 0.742263 |
| 3344 | −0.3509 | −0.33426 | 0 | 0 | 0 | 0.230316 | 7 | 537.0978 | 0.740099 |
| 3345 | −0.35435 | 0 | 0.326983 | 0 | 0.276248 | 0 | 7 | 546.3488 | 0.721623 |
| 3346 | −0.35429 | 0 | 0.335115 | 0 | 0 | 0.278056 | 7 | 547.4068 | 0.721705 |
| 3347 | −0.35918 | 0 | 0 | 0.257296 | 0.231108 | 0 | 7 | 544.1727 | 0.730116 |
| 3348 | −0.36046 | 0 | 0 | 0.27483 | 0 | 0.241127 | 7 | 544.5247 | 0.729463 |
| 3349 | −0.34604 | 0 | 0 | 0 | 0.20186 | 0.189915 | 7 | 545.7914 | 0.72589 |
| 3350 | −0.34954 | 0 | 0.311734 | 0 | 0 | 0 | 7 | 550.0492 | 0.715315 |
| 3351 | −0.34838 | −0.35299 | 0 | 0 | 0 | 0 | 7 | 537.1481 | 0.740017 |
| 3352 | −0.35731 | 0 | 0 | 0.283735 | 0 | 0 | 7 | 545.7277 | 0.725523 |
| 3353 | −0.34421 | 0 | 0 | 0 | 0.260301 | 0 | 7 | 544.8525 | 0.723889 |
| 3354 | −0.34349 | 0 | 0 | 0 | 0 | 0.25144 | 7 | 544.0344 | 0.727299 |
| 3355 | −0.3635 | 0 | 0.280897 | 0.247949 | 0 | 0 | 7 | 548.8584 | 0.718684 |
| 3356 | −0.36484 | −0.51223 | 0.448932 | 0 | 0 | 0 | 7 | 534.548 | 0.742814 |
| 3357 | −0.36535 | −0.34122 | 0 | 0.272545 | 0 | 0 | 7 | 536.696 | 0.742528 |
| 3358 | −0.35247 | −0.33749 | 0 | 0 | 0.244058 | 0 | 7 | 536.6104 | 0.738629 |
| 3359 | −0.3509 | −0.33426 | 0 | 0 | 0 | 0.230316 | 7 | 537.3016 | 0.738629 |
| 3360 | −0.35435 | 0 | 0.326983 | 0 | 0.276248 | 0 | 7 | 547.1337 | 0.719704 |
| 3361 | −0.35429 | 0 | 0.335115 | 0 | 0 | 0.278056 | 7 | 547.6218 | 0.720215 |
| 3362 | −0.35918 | 0 | 0 | 0.257296 | 0.231108 | 0 | 7 | 544.9189 | 0.725114 |
| 3363 | −0.36046 | 0 | 0 | 0.27483 | 0 | 0.241127 | 7 | 544.7628 | 0.727176 |
| 3364 | −0.34604 | 0 | 0 | 0 | 0.20186 | 0.189915 | 7 | 546.3649 | 0.722624 |
| 3365 | −0.3635 | 0 | 0.280897 | 0.247949 | 0 | 0 | 7 | 545.8962 | 0.725747 |
| 3366 | −0.36484 | −0.51223 | 0.448932 | 0 | 0 | 0 | 7 | 531.0857 | 0.748714 |
| 3367 | −0.36535 | −0.34122 | 0 | 0.272545 | 0 | 0 | 7 | 532.2514 | 0.750327 |
| 3368 | −0.35247 | −0.33749 | 0 | 0 | 0.244058 | 0 | 7 | 531.8785 | 0.746285 |
| 3369 | −0.3509 | −0.33426 | 0 | 0 | 0 | 0.230316 | 7 | 531.0687 | 0.748612 |
| 3370 | −0.35435 | 0 | 0.326983 | 0 | 0.276248 | 0 | 7 | 543.8505 | 0.725584 |
| 3371 | −0.35429 | 0 | 0.335115 | 0 | 0 | 0.278056 | 7 | 542.9718 | 0.72785 |
| 3372 | −0.35918 | 0 | 0 | 0.257296 | 0.231108 | 0 | 7 | 540.8218 | 0.734342 |
| 3373 | −0.36046 | 0 | 0 | 0.27483 | 0 | 0.241127 | 7 | 539.1695 | 0.736485 |
| 3374 | −0.34604 | 0 | 0 | 0 | 0.20186 | 0.189915 | 7 | 541.1138 | 0.731708 |
| 3375 | −0.37665 | −0.49016 | 0.415897 | 0.213769 | 0 | 0 | 7 | 531.4909 | 0.747999 |
| 3376 | −0.3692 | −0.49878 | 0.458935 | 0 | 0.257995 | 0 | 7 | 529.5489 | 0.748346 |
| 3377 | −0.36789 | −0.4958 | 0.464972 | 0 | 0 | 0.255069 | 7 | 530.2672 | 0.74655 |
| 3378 | −0.36683 | −0.32775 | 0 | 0.247282 | 0.215748 | 0 | 7 | 533.1885 | 0.746917 |
| 3379 | −0.36769 | −0.32552 | 0 | 0.266766 | 0 | 0.223296 | 7 | 533.2418 | 0.74804 |
| 3380 | −0.35346 | −0.32601 | 0 | 0 | 0.190682 | 0.171912 | 7 | 534.9925 | 0.742426 |
| 3381 | −0.36573 | 0 | 0.297911 | 0.217151 | 0.249518 | 0 | 7 | 544.2238 | 0.726666 |
| 3382 | −0.36751 | 0 | 0.304766 | 0.234822 | 0 | 0.26618 | 7 | 544.1456 | 0.72834 |
| 3383 | −0.35702 | 0 | 0.341819 | 0 | 0.21209 | 0.213821 | 7 | 544.6282 | 0.724053 |
| 3384 | −0.36116 | 0 | 0 | 0.256551 | 0.172681 | 0.188346 | 7 | 542.8077 | 0.729912 |
| 3385 | −0.34954 | 0 | 0.311734 | 0 | 0 | 0 | 7 | 577.1891 | 0.66291 |
| 3386 | −0.34838 | −0.35299 | 0 | 0 | 0 | 0 | 7 | 578.1347 | 0.658582 |
| 3387 | −0.35731 | 0 | 0 | 0.283735 | 0 | 0 | 7 | 580.2101 | 0.659909 |
| 3388 | −0.34421 | 0 | 0 | 0 | 0.260301 | 0 | 7 | 579.522 | 0.666197 |
| 3389 | −0.34349 | 0 | 0 | 0 | 0 | 0.25144 | 7 | 581.166 | 0.655418 |
| 3390 | −0.34954 | 0 | 0.311734 | 0 | 0 | 0 | 7 | 576.6553 | 0.665768 |
| 3391 | −0.34838 | −0.35299 | 0 | 0 | 0 | 0 | 7 | 577.4946 | 0.662747 |
| 3392 | −0.35731 | 0 | 0 | 0.283735 | 0 | 0 | 7 | 579.5528 | 0.660379 |
| 3393 | −0.34421 | 0 | 0 | 0 | 0.260301 | 0 | 7 | 579.495 | 0.6673 |
| 3394 | −0.34349 | 0 | 0 | 0 | 0 | 0.25144 | 7 | 580.4019 | 0.659032 |
| 3395 | −0.34954 | 0 | 0.311734 | 0 | 0 | 0 | 7 | 574.8505 | 0.666238 |
| 3396 | −0.34838 | −0.35299 | 0 | 0 | 0 | 0 | 7 | 574.6025 | 0.66587 |
| 3397 | −0.35731 | 0 | 0 | 0.283735 | 0 | 0 | 7 | 577.061 | 0.666054 |
| 3398 | −0.34421 | 0 | 0 | 0 | 0.260301 | 0 | 7 | 576.4447 | 0.665258 |
| 3399 | −0.34349 | 0 | 0 | 0 | 0 | 0.25144 | 7 | 575.9112 | 0.664809 |
| 3400 | −0.3635 | 0 | 0.280897 | 0.247949 | 0 | 0 | 7 | 572.8512 | 0.672975 |
| 3401 | −0.36484 | −0.51223 | 0.448932 | 0 | 0 | 0 | 7 | 560.9036 | 0.69192 |
| 3402 | −0.36535 | −0.34122 | 0 | 0.272545 | 0 | 0 | 7 | 572.5556 | 0.671995 |
| 3403 | −0.35247 | −0.33749 | 0 | 0 | 0.244058 | 0 | 7 | 572.7307 | 0.67522 |
| 3404 | −0.3509 | −0.33426 | 0 | 0 | 0 | 0.230316 | 7 | 573.8723 | 0.668157 |
| 3405 | −0.35435 | 0 | 0.326983 | 0 | 0.276248 | 0 | 7 | 570.0288 | 0.678058 |
| 3406 | −0.35429 | 0 | 0.335115 | 0 | 0 | 0.278056 | 7 | 570.8723 | 0.676017 |
| 3407 | −0.35918 | 0 | 0 | 0.257296 | 0.231108 | 0 | 7 | 575.3013 | 0.669953 |
| 3408 | −0.36046 | 0 | 0 | 0.27483 | 0 | 0.241127 | 7 | 575.4409 | 0.665054 |
| 3409 | −0.34604 | 0 | 0 | 0 | 0.20186 | 0.189915 | 7 | 577.807 | 0.664013 |
| 3410 | −0.34954 | 0 | 0.311734 | 0 | 0 | 0 | 7 | 576.9609 | 0.664911 |
| 3411 | −0.34838 | −0.35299 | 0 | 0 | 0 | 0 | 7 | 577.8333 | 0.662686 |
| 3412 | −0.35731 | 0 | 0 | 0.283735 | 0 | 0 | 7 | 579.5716 | 0.65993 |
| 3413 | −0.34421 | 0 | 0 | 0 | 0.260301 | 0 | 7 | 578.9778 | 0.668484 |
| 3414 | −0.34349 | 0 | 0 | 0 | 0 | 0.25144 | 7 | 580.2901 | 0.660236 |
| 3415 | −0.34954 | 0 | 0.311734 | 0 | 0 | 0 | 7 | 575.6226 | 0.665013 |
| 3416 | −0.34838 | −0.35299 | 0 | 0 | 0 | 0 | 7 | 575.4873 | 0.666238 |
| 3417 | −0.35731 | 0 | 0 | 0.283735 | 0 | 0 | 7 | 577.5563 | 0.663707 |
| 3418 | −0.34421 | 0 | 0 | 0 | 0.260301 | 0 | 7 | 576.6589 | 0.666299 |
| 3419 | −0.34349 | 0 | 0 | 0 | 0 | 0.25144 | 7 | 576.4539 | 0.66536 |
| 3420 | −0.3635 | 0 | 0.280897 | 0.247949 | 0 | 0 | 7 | 572.7129 | 0.674873 |
| 3421 | −0.36484 | −0.51223 | 0.448932 | 0 | 0 | 0 | 7 | 561.0443 | 0.692736 |
| 3422 | −0.36535 | −0.34122 | 0 | 0.272545 | 0 | 0 | 7 | 572.4244 | 0.673791 |
| 3423 | −0.35247 | −0.33749 | 0 | 0 | 0.244058 | 0 | 7 | 572.0725 | 0.678671 |
| 3424 | −0.3509 | −0.33426 | 0 | 0 | 0 | 0.230316 | 7 | 573.6516 | 0.669545 |
| 3425 | −0.35435 | 0 | 0.326983 | 0 | 0.276248 | 0 | 7 | 569.1067 | 0.680304 |
| 3426 | −0.35429 | 0 | 0.335115 | 0 | 0 | 0.278056 | 7 | 570.4918 | 0.677813 |
| 3427 | −0.35918 | 0 | 0 | 0.257296 | 0.231108 | 0 | 7 | 574.3918 | 0.673363 |
| 3428 | −0.36046 | 0 | 0 | 0.27483 | 0 | 0.241127 | 7 | 574.9351 | 0.667361 |
| 3429 | −0.34604 | 0 | 0 | 0 | 0.20186 | 0.189915 | 7 | 576.9756 | 0.667034 |
| 3430 | −0.34954 | 0 | 0.311734 | 0 | 0 | 0 | 7 | 574.9862 | 0.668116 |
| 3431 | −0.34838 | −0.35299 | 0 | 0 | 0 | 0 | 7 | 574.8309 | 0.669933 |
| 3432 | −0.35731 | 0 | 0 | 0.283735 | 0 | 0 | 7 | 576.7987 | 0.666136 |
| 3433 | −0.34421 | 0 | 0 | 0 | 0.260301 | 0 | 7 | 576.2235 | 0.669484 |
| 3434 | −0.34349 | 0 | 0 | 0 | 0 | 0.25144 | 7 | 575.5364 | 0.669076 |
| 3435 | −0.3635 | 0 | 0.280897 | 0.247949 | 0 | 0 | 7 | 572.2903 | 0.673424 |
| 3436 | −0.36484 | −0.51223 | 0.448932 | 0 | 0 | 0 | 7 | 560.8875 | 0.693553 |
| 3437 | −0.36535 | −0.34122 | 0 | 0.272545 | 0 | 0 | 7 | 571.9254 | 0.6752 |
| 3438 | −0.35247 | −0.33749 | 0 | 0 | 0.244058 | 0 | 7 | 572.088 | 0.680671 |
| 3439 | −0.3509 | −0.33426 | 0 | 0 | 0 | 0.230316 | 7 | 573.0721 | 0.672709 |
| 3440 | −0.35435 | 0 | 0.326983 | 0 | 0.276248 | 0 | 7 | 569.4761 | 0.679161 |
| 3441 | −0.35429 | 0 | 0.335115 | 0 | 0 | 0.278056 | 7 | 570.0907 | 0.678773 |
| 3442 | −0.35918 | 0 | 0 | 0.257296 | 0.231108 | 0 | 7 | 574.4325 | 0.67222 |
| 3443 | −0.36046 | 0 | 0 | 0.27483 | 0 | 0.241127 | 7 | 574.3822 | 0.667912 |
| 3444 | −0.34604 | 0 | 0 | 0 | 0.20186 | 0.189915 | 7 | 576.8158 | 0.668177 |
| 3445 | −0.3635 | 0 | 0.280897 | 0.247949 | 0 | 0 | 7 | 570.508 | 0.678752 |
| 3446 | −0.36484 | −0.51223 | 0.448932 | 0 | 0 | 0 | 7 | 558.6586 | 0.696513 |
| 3447 | −0.36535 | −0.34122 | 0 | 0.272545 | 0 | 0 | 7 | 569.0485 | 0.680712 |
| 3448 | −0.35247 | −0.33749 | 0 | 0 | 0.244058 | 0 | 7 | 568.7738 | 0.682019 |
| 3449 | −0.3509 | −0.33426 | 0 | 0 | 0 | 0.230316 | 7 | 568.402 | 0.679548 |
| 3450 | −0.35435 | 0 | 0.326983 | 0 | 0.276248 | 0 | 7 | 567.1169 | 0.680406 |
| 3451 | −0.35429 | 0 | 0.335115 | 0 | 0 | 0.278056 | 7 | 566.0608 | 0.684203 |
| 3452 | −0.35918 | 0 | 0 | 0.257296 | 0.231108 | 0 | 7 | 571.4276 | 0.674935 |
| 3453 | −0.36046 | 0 | 0 | 0.27483 | 0 | 0.241127 | 7 | 569.8112 | 0.678079 |
| 3454 | −0.34604 | 0 | 0 | 0 | 0.20186 | 0.189915 | 7 | 572.4527 | 0.672914 |
| 3455 | −0.37665 | −0.49016 | 0.415897 | 0.213769 | 0 | 0 | 7 | 557.9374 | 0.697697 |
| 3456 | −0.3692 | −0.49878 | 0.458935 | 0 | 0.257995 | 0 | 7 | 554.7216 | 0.701556 |
| 3457 | −0.36789 | −0.4958 | 0.464972 | 0 | 0 | 0.255069 | 7 | 555.6232 | 0.698779 |
| 3458 | −0.36683 | −0.32775 | 0 | 0.247282 | 0.215748 | 0 | 7 | 568.0911 | 0.683223 |
| 3459 | −0.36769 | −0.32552 | 0 | 0.266766 | 0 | 0.223296 | 7 | 568.2355 | 0.676813 |
| 3460 | −0.35346 | −0.32601 | 0 | 0 | 0.190682 | 0.171912 | 7 | 570.6138 | 0.675874 |
| 3461 | −0.36573 | 0 | 0.297911 | 0.217151 | 0.249518 | 0 | 7 | 566.7072 | 0.685714 |
| 3462 | −0.36751 | 0 | 0.304766 | 0.234822 | 0 | 0.26618 | 7 | 566.6606 | 0.683162 |
| 3463 | −0.35702 | 0 | 0.341819 | 0 | 0.21209 | 0.213821 | 7 | 566.6466 | 0.682366 |
| 3464 | −0.36116 | 0 | 0 | 0.256551 | 0.172681 | 0.188346 | 7 | 572.5991 | 0.670913 |
| 3465 | −0.34954 | 0 | 0.311734 | 0 | 0 | 0 | 7 | 578.4992 | 0.662482 |
| 3466 | −0.34838 | −0.35299 | 0 | 0 | 0 | 0 | 7 | 578.5011 | 0.661277 |
| 3467 | −0.35731 | 0 | 0 | 0.283735 | 0 | 0 | 7 | 580.5208 | 0.658766 |
| 3468 | −0.34421 | 0 | 0 | 0 | 0.260301 | 0 | 7 | 580.164 | 0.669586 |
| 3469 | −0.34349 | 0 | 0 | 0 | 0 | 0.25144 | 7 | 581.3285 | 0.658848 |
| 3470 | −0.34954 | 0 | 0.311734 | 0 | 0 | 0 | 7 | 576.6962 | 0.665564 |
| 3471 | −0.34838 | −0.35299 | 0 | 0 | 0 | 0 | 7 | 575.7049 | 0.665278 |
| 3472 | −0.35731 | 0 | 0 | 0.283735 | 0 | 0 | 7 | 578.1282 | 0.664686 |
| 3473 | −0.34421 | 0 | 0 | 0 | 0.260301 | 0 | 7 | 577.37 | 0.66632 |
| 3474 | −0.34349 | 0 | 0 | 0 | 0 | 0.25144 | 7 | 577.0769 | 0.665258 |
| 3475 | −0.3635 | 0 | 0.280897 | 0.247949 | 0 | 0 | 7 | 574.0862 | 0.672383 |
| 3476 | −0.36484 | −0.51223 | 0.448932 | 0 | 0 | 0 | 7 | 562.4368 | 0.692287 |
| 3477 | −0.36535 | −0.34122 | 0 | 0.272545 | 0 | 0 | 7 | 573.0723 | 0.67522 |
| 3478 | −0.35247 | −0.33749 | 0 | 0 | 0.244058 | 0 | 7 | 572.9441 | 0.67767 |
| 3479 | −0.3509 | −0.33426 | 0 | 0 | 0 | 0.230316 | 7 | 574.3614 | 0.667483 |
| 3480 | −0.35435 | 0 | 0.326983 | 0 | 0.276248 | 0 | 7 | 570.9918 | 0.678936 |
| 3481 | −0.35429 | 0 | 0.335115 | 0 | 0 | 0.278056 | 7 | 572.1173 | 0.673751 |
| 3482 | −0.35918 | 0 | 0 | 0.257296 | 0.231108 | 0 | 7 | 575.456 | 0.672056 |
| 3483 | −0.36046 | 0 | 0 | 0.27483 | 0 | 0.241127 | 7 | 575.8331 | 0.666115 |
| 3484 | −0.34604 | 0 | 0 | 0 | 0.20186 | 0.189915 | 7 | 578.0245 | 0.66832 |
| 3485 | −0.34954 | 0 | 0.311734 | 0 | 0 | 0 | 7 | 576.3943 | 0.66734 |
| 3486 | −0.34838 | −0.35299 | 0 | 0 | 0 | 0 | 7 | 575.2857 | 0.669239 |
| 3487 | −0.35731 | 0 | 0 | 0.283735 | 0 | 0 | 7 | 577.6625 | 0.666381 |
| 3488 | −0.34421 | 0 | 0 | 0 | 0.260301 | 0 | 7 | 577.4658 | 0.668667 |
| 3489 | −0.34349 | 0 | 0 | 0 | 0 | 0.25144 | 7 | 576.5005 | 0.667075 |
| 3490 | −0.3635 | 0 | 0.280897 | 0.247949 | 0 | 0 | 7 | 574.103 | 0.672117 |
| 3491 | −0.36484 | −0.51223 | 0.448932 | 0 | 0 | 0 | 7 | 562.7624 | 0.69241 |
| 3492 | −0.36535 | −0.34122 | 0 | 0.272545 | 0 | 0 | 7 | 572.9489 | 0.676792 |
| 3493 | −0.35247 | −0.33749 | 0 | 0 | 0.244058 | 0 | 7 | 573.5127 | 0.679508 |
| 3494 | −0.3509 | −0.33426 | 0 | 0 | 0 | 0.230316 | 7 | 574.1917 | 0.669811 |
| 3495 | −0.35435 | 0 | 0.326983 | 0 | 0.276248 | 0 | 7 | 571.9993 | 0.676772 |
| 3496 | −0.35429 | 0 | 0.335115 | 0 | 0 | 0.278056 | 7 | 572.2216 | 0.67373 |
| 3497 | −0.35918 | 0 | 0 | 0.257296 | 0.231108 | 0 | 7 | 576.018 | 0.670362 |
| 3498 | −0.36046 | 0 | 0 | 0.27483 | 0 | 0.241127 | 7 | 575.6912 | 0.664584 |
| 3499 | −0.34604 | 0 | 0 | 0 | 0.20186 | 0.189915 | 7 | 578.3932 | 0.666279 |
| 3500 | −0.3635 | 0 | 0.280897 | 0.247949 | 0 | 0 | 7 | 571.8752 | 0.677017 |
| 3501 | −0.36484 | −0.51223 | 0.448932 | 0 | 0 | 0 | 7 | 560.06 | 0.698167 |
| 3502 | −0.36535 | −0.34122 | 0 | 0.272545 | 0 | 0 | 7 | 569.6669 | 0.682856 |
| 3503 | −0.35247 | −0.33749 | 0 | 0 | 0.244058 | 0 | 7 | 569.7447 | 0.682896 |
| 3504 | −0.3509 | −0.33426 | 0 | 0 | 0 | 0.230316 | 7 | 569.0727 | 0.678916 |
| 3505 | −0.35435 | 0 | 0.326983 | 0 | 0.276248 | 0 | 7 | 569.1681 | 0.677201 |
| 3506 | −0.35429 | 0 | 0.335115 | 0 | 0 | 0.278056 | 7 | 567.7789 | 0.680508 |
| 3507 | −0.35918 | 0 | 0 | 0.257296 | 0.231108 | 0 | 7 | 572.6383 | 0.672485 |
| 3508 | −0.36046 | 0 | 0 | 0.27483 | 0 | 0.241127 | 7 | 570.8169 | 0.675731 |
| 3509 | −0.34604 | 0 | 0 | 0 | 0.20186 | 0.189915 | 7 | 573.6468 | 0.669708 |
| 3510 | −0.37665 | −0.49016 | 0.415897 | 0.213769 | 0 | 0 | 7 | 559.6419 | 0.697105 |
| 3511 | −0.3692 | −0.49878 | 0.458935 | 0 | 0.257995 | 0 | 7 | 557.1655 | 0.700698 |
| 3512 | −0.36789 | −0.4958 | 0.464972 | 0 | 0 | 0.255069 | 7 | 557.5985 | 0.696084 |
| 3513 | −0.36683 | −0.32775 | 0 | 0.247282 | 0.215748 | 0 | 7 | 569.3016 | 0.684428 |
| 3514 | −0.36769 | −0.32552 | 0 | 0.266766 | 0 | 0.223296 | 7 | 569.1772 | 0.677466 |
| 3515 | −0.35346 | −0.32601 | 0 | 0 | 0.190682 | 0.171912 | 7 | 571.8174 | 0.677487 |
| 3516 | −0.36573 | 0 | 0.297911 | 0.217151 | 0.249518 | 0 | 7 | 568.8401 | 0.682468 |
| 3517 | −0.36751 | 0 | 0.304766 | 0.234822 | 0 | 0.26618 | 7 | 568.4028 | 0.679018 |
| 3518 | −0.35702 | 0 | 0.341819 | 0 | 0.21209 | 0.213821 | 7 | 568.9067 | 0.679691 |
| 3519 | −0.36116 | 0 | 0 | 0.256551 | 0.172681 | 0.188346 | 7 | 573.8709 | 0.668831 |
| 3520 | −0.34954 | 0 | 0.311734 | 0 | 0 | 0 | 7 | 505.6349 | 0.780214 |
| 3521 | −0.34838 | −0.35299 | 0 | 0 | 0 | 0 | 7 | 501.671 | 0.784134 |
| 3522 | −0.35731 | 0 | 0 | 0.283735 | 0 | 0 | 7 | 505.9751 | 0.77707 |
| 3523 | −0.34421 | 0 | 0 | 0 | 0.260301 | 0 | 7 | 506.2196 | 0.77707 |
| 3524 | −0.34349 | 0 | 0 | 0 | 0 | 0.25144 | 7 | 505.9459 | 0.778989 |
| 3525 | −0.34954 | 0 | 0.311734 | 0 | 0 | 0 | 7 | 508.8023 | 0.775457 |
| 3526 | −0.34838 | −0.35299 | 0 | 0 | 0 | 0 | 7 | 504.741 | 0.778703 |
| 3527 | −0.35731 | 0 | 0 | 0.283735 | 0 | 0 | 7 | 508.5519 | 0.774334 |
| 3528 | −0.34421 | 0 | 0 | 0 | 0.260301 | 0 | 7 | 509.1419 | 0.773069 |
| 3529 | −0.34349 | 0 | 0 | 0 | 0 | 0.25144 | 7 | 508.612 | 0.775029 |
| 3530 | −0.34954 | 0 | 0.311734 | 0 | 0 | 0 | 7 | 507.2967 | 0.778499 |
| 3531 | −0.34838 | −0.35299 | 0 | 0 | 0 | 0 | 7 | 503.3953 | 0.781051 |
| 3532 | −0.35731 | 0 | 0 | 0.283735 | 0 | 0 | 7 | 507.0394 | 0.775927 |
| 3533 | −0.34421 | 0 | 0 | 0 | 0.260301 | 0 | 7 | 507.0959 | 0.77756 |
| 3534 | −0.34349 | 0 | 0 | 0 | 0 | 0.25144 | 7 | 507.0641 | 0.777438 |
| 3535 | −0.34954 | 0 | 0.311734 | 0 | 0 | 0 | 7 | 508.3373 | 0.775723 |
| 3536 | −0.34838 | −0.35299 | 0 | 0 | 0 | 0 | 7 | 504.2642 | 0.778907 |
| 3537 | −0.35731 | 0 | 0 | 0.283735 | 0 | 0 | 7 | 508.0413 | 0.773702 |
| 3538 | −0.34421 | 0 | 0 | 0 | 0.260301 | 0 | 7 | 508.6215 | 0.773885 |
| 3539 | −0.34349 | 0 | 0 | 0 | 0 | 0.25144 | 7 | 508.0425 | 0.775498 |
| 3540 | −0.34954 | 0 | 0.311734 | 0 | 0 | 0 | 7 | 505.1942 | 0.78099 |
| 3541 | −0.34838 | −0.35299 | 0 | 0 | 0 | 0 | 7 | 499.8744 | 0.785665 |
| 3542 | −0.35731 | 0 | 0 | 0.283735 | 0 | 0 | 7 | 504.1594 | 0.780826 |
| 3543 | −0.34421 | 0 | 0 | 0 | 0.260301 | 0 | 7 | 504.5328 | 0.781541 |
| 3544 | −0.34349 | 0 | 0 | 0 | 0 | 0.25144 | 7 | 503.0635 | 0.782439 |
| 3545 | −0.3635 | 0 | 0.280897 | 0.247949 | 0 | 0 | 7 | 506.1779 | 0.777866 |
| 3546 | −0.36484 | −0.51223 | 0.448932 | 0 | 0 | 0 | 7 | 497.3092 | 0.790258 |
| 3547 | −0.36535 | −0.34122 | 0 | 0.272545 | 0 | 0 | 7 | 501.6297 | 0.781235 |
| 3548 | −0.35247 | −0.33749 | 0 | 0 | 0.244058 | 0 | 7 | 502.3952 | 0.780847 |
| 3549 | −0.3509 | −0.33426 | 0 | 0 | 0 | 0.230316 | 7 | 502.0082 | 0.782092 |
| 3550 | −0.35435 | 0 | 0.326983 | 0 | 0.276248 | 0 | 7 | 505.973 | 0.777152 |
| 3551 | −0.35429 | 0 | 0.335115 | 0 | 0 | 0.278056 | 7 | 505.186 | 0.778764 |
| 3552 | −0.35918 | 0 | 0 | 0.257296 | 0.231108 | 0 | 7 | 506.4449 | 0.775804 |
| 3553 | −0.36046 | 0 | 0 | 0.27483 | 0 | 0.241127 | 7 | 505.5597 | 0.777417 |
| 3554 | −0.34604 | 0 | 0 | 0 | 0.20186 | 0.189915 | 7 | 507.3186 | 0.776315 |
| 3555 | −0.34954 | 0 | 0.311734 | 0 | 0 | 0 | 7 | 544.2554 | 0.72636 |
| 3556 | −0.34838 | −0.35299 | 0 | 0 | 0 | 0 | 7 | 552.5461 | 0.711334 |
| 3557 | −0.35731 | 0 | 0 | 0.283735 | 0 | 0 | 7 | 550.8375 | 0.715111 |
| 3558 | −0.34421 | 0 | 0 | 0 | 0.260301 | 0 | 7 | 551.6055 | 0.719112 |
| 3559 | −0.34349 | 0 | 0 | 0 | 0 | 0.25144 | 7 | 551.3293 | 0.716132 |
| 3560 | −0.34954 | 0 | 0.311734 | 0 | 0 | 0 | 7 | 543.458 | 0.729871 |
| 3561 | −0.34838 | −0.35299 | 0 | 0 | 0 | 0 | 7 | 551.9777 | 0.712253 |
| 3562 | −0.35731 | 0 | 0 | 0.283735 | 0 | 0 | 7 | 550.0995 | 0.718071 |
| 3563 | −0.34421 | 0 | 0 | 0 | 0.260301 | 0 | 7 | 550.2423 | 0.719929 |
| 3564 | −0.34349 | 0 | 0 | 0 | 0 | 0.25144 | 7 | 550.5378 | 0.716479 |
| 3565 | −0.34954 | 0 | 0.311734 | 0 | 0 | 0 | 7 | 543.7406 | 0.728034 |
| 3566 | −0.34838 | −0.35299 | 0 | 0 | 0 | 0 | 7 | 551.9309 | 0.713212 |
| 3567 | −0.35731 | 0 | 0 | 0.283735 | 0 | 0 | 7 | 550.1366 | 0.717785 |
| 3568 | −0.34421 | 0 | 0 | 0 | 0.260301 | 0 | 7 | 550.9794 | 0.720827 |
| 3569 | −0.34349 | 0 | 0 | 0 | 0 | 0.25144 | 7 | 550.5922 | 0.717806 |
| 3570 | −0.34954 | 0 | 0.311734 | 0 | 0 | 0 | 7 | 542.0438 | 0.731423 |
| 3571 | −0.34838 | −0.35299 | 0 | 0 | 0 | 0 | 7 | 549.3686 | 0.717255 |
| 3572 | −0.35731 | 0 | 0 | 0.283735 | 0 | 0 | 7 | 547.7554 | 0.722562 |
| 3573 | −0.34421 | 0 | 0 | 0 | 0.260301 | 0 | 7 | 548.2339 | 0.721317 |
| 3574 | −0.34349 | 0 | 0 | 0 | 0 | 0.25144 | 7 | 546.8109 | 0.724175 |
| 3575 | −0.3635 | 0 | 0.280897 | 0.247949 | 0 | 0 | 7 | 541.2444 | 0.73179 |
| 3576 | −0.36484 | −0.51223 | 0.448932 | 0 | 0 | 0 | 7 | 535.3979 | 0.738139 |
| 3577 | −0.36535 | −0.34122 | 0 | 0.272545 | 0 | 0 | 7 | 548.2569 | 0.71799 |
| 3578 | −0.35247 | −0.33749 | 0 | 0 | 0.244058 | 0 | 7 | 548.9376 | 0.719664 |
| 3579 | −0.3509 | −0.33426 | 0 | 0 | 0 | 0.230316 | 7 | 548.8995 | 0.716928 |
| 3580 | −0.35435 | 0 | 0.326983 | 0 | 0.276248 | 0 | 7 | 539.8249 | 0.731525 |
| 3581 | −0.35429 | 0 | 0.335115 | 0 | 0 | 0.278056 | 7 | 539.0099 | 0.735077 |
| 3582 | −0.35918 | 0 | 0 | 0.257296 | 0.231108 | 0 | 7 | 547.8911 | 0.719949 |
| 3583 | −0.36046 | 0 | 0 | 0.27483 | 0 | 0.241127 | 7 | 547.0349 | 0.719847 |
| 3584 | −0.34604 | 0 | 0 | 0 | 0.20186 | 0.189915 | 7 | 549.4322 | 0.719357 |
| 3585 | −0.34954 | 0 | 0.311734 | 0 | 0 | 0 | 7 | 544.3812 | 0.727442 |
| 3586 | −0.34838 | −0.35299 | 0 | 0 | 0 | 0 | 7 | 552.3694 | 0.712396 |
| 3587 | −0.35731 | 0 | 0 | 0.283735 | 0 | 0 | 7 | 550.501 | 0.71801 |
| 3588 | −0.34421 | 0 | 0 | 0 | 0.260301 | 0 | 7 | 550.818 | 0.718439 |
| 3589 | −0.34349 | 0 | 0 | 0 | 0 | 0.25144 | 7 | 550.9229 | 0.71554 |
| 3590 | −0.34954 | 0 | 0.311734 | 0 | 0 | 0 | 7 | 544.978 | 0.725461 |
| 3591 | −0.34838 | −0.35299 | 0 | 0 | 0 | 0 | 7 | 552.5637 | 0.711722 |
| 3592 | −0.35731 | 0 | 0 | 0.283735 | 0 | 0 | 7 | 550.7644 | 0.716683 |
| 3593 | −0.34421 | 0 | 0 | 0 | 0.260301 | 0 | 7 | 551.8183 | 0.717724 |
| 3594 | −0.34349 | 0 | 0 | 0 | 0 | 0.25144 | 7 | 551.1946 | 0.715376 |
| 3595 | −0.34954 | 0 | 0.311734 | 0 | 0 | 0 | 7 | 543.1589 | 0.728115 |
| 3596 | −0.34838 | −0.35299 | 0 | 0 | 0 | 0 | 7 | 549.8629 | 0.71456 |
| 3597 | −0.35731 | 0 | 0 | 0.283735 | 0 | 0 | 7 | 548.3384 | 0.719888 |
| 3598 | −0.34421 | 0 | 0 | 0 | 0.260301 | 0 | 7 | 549.0533 | 0.719602 |
| 3599 | −0.34349 | 0 | 0 | 0 | 0 | 0.25144 | 7 | 547.4539 | 0.723175 |
| 3600 | −0.3635 | 0 | 0.280897 | 0.247949 | 0 | 0 | 7 | 542.1207 | 0.728687 |
| 3601 | −0.36484 | −0.51223 | 0.448932 | 0 | 0 | 0 | 7 | 536.6404 | 0.73426 |
| 3602 | −0.36535 | −0.34122 | 0 | 0.272545 | 0 | 0 | 7 | 548.6675 | 0.71799 |
| 3603 | −0.35247 | −0.33749 | 0 | 0 | 0.244058 | 0 | 7 | 549.5495 | 0.719153 |
| 3604 | −0.3509 | −0.33426 | 0 | 0 | 0 | 0.230316 | 7 | 549.2907 | 0.715356 |
| 3605 | −0.35435 | 0 | 0.326983 | 0 | 0.276248 | 0 | 7 | 541.1069 | 0.730871 |
| 3606 | −0.35429 | 0 | 0.335115 | 0 | 0 | 0.278056 | 7 | 539.9106 | 0.732157 |
| 3607 | −0.35918 | 0 | 0 | 0.257296 | 0.231108 | 0 | 7 | 548.3822 | 0.719582 |
| 3608 | −0.36046 | 0 | 0 | 0.27483 | 0 | 0.241127 | 7 | 547.2402 | 0.720337 |
| 3609 | −0.34604 | 0 | 0 | 0 | 0.20186 | 0.189915 | 7 | 549.8968 | 0.717336 |
| 3610 | −0.34954 | 0 | 0.311734 | 0 | 0 | 0 | 7 | 544.1478 | 0.728319 |
| 3611 | −0.34838 | −0.35299 | 0 | 0 | 0 | 0 | 7 | 551.9911 | 0.713457 |
| 3612 | −0.35731 | 0 | 0 | 0.283735 | 0 | 0 | 7 | 549.9609 | 0.718929 |
| 3613 | −0.34421 | 0 | 0 | 0 | 0.260301 | 0 | 7 | 550.3771 | 0.719949 |
| 3614 | −0.34349 | 0 | 0 | 0 | 0 | 0.25144 | 7 | 550.3102 | 0.716867 |
| 3615 | −0.34954 | 0 | 0.311734 | 0 | 0 | 0 | 7 | 542.7905 | 0.730259 |
| 3616 | −0.34838 | −0.35299 | 0 | 0 | 0 | 0 | 7 | 549.7683 | 0.716479 |
| 3617 | −0.35731 | 0 | 0 | 0.283735 | 0 | 0 | 7 | 547.9542 | 0.722889 |
| 3618 | −0.34421 | 0 | 0 | 0 | 0.260301 | 0 | 7 | 548.2205 | 0.720521 |
| 3619 | −0.34349 | 0 | 0 | 0 | 0 | 0.25144 | 7 | 547.0866 | 0.724359 |
| 3620 | −0.3635 | 0 | 0.280897 | 0.247949 | 0 | 0 | 7 | 541.0864 | 0.732178 |
| 3621 | −0.36484 | −0.51223 | 0.448932 | 0 | 0 | 0 | 7 | 535.7449 | 0.737179 |
| 3622 | −0.36535 | −0.34122 | 0 | 0.272545 | 0 | 0 | 7 | 547.8686 | 0.720317 |
| 3623 | −0.35247 | −0.33749 | 0 | 0 | 0.244058 | 0 | 7 | 548.1636 | 0.722869 |
| 3624 | −0.3509 | −0.33426 | 0 | 0 | 0 | 0.230316 | 7 | 548.4387 | 0.717398 |
| 3625 | −0.35435 | 0 | 0.326983 | 0 | 0.276248 | 0 | 7 | 539.1158 | 0.734954 |
| 3626 | −0.35429 | 0 | 0.335115 | 0 | 0 | 0.278056 | 7 | 538.6887 | 0.734403 |
| 3627 | −0.35918 | 0 | 0 | 0.257296 | 0.231108 | 0 | 7 | 546.8049 | 0.72291 |
| 3628 | −0.36046 | 0 | 0 | 0.27483 | 0 | 0.241127 | 7 | 546.2225 | 0.722481 |
| 3629 | −0.34604 | 0 | 0 | 0 | 0.20186 | 0.189915 | 7 | 548.3906 | 0.720521 |
| 3630 | −0.34954 | 0 | 0.311734 | 0 | 0 | 0 | 7 | 543.0101 | 0.729361 |
| 3631 | −0.34838 | −0.35299 | 0 | 0 | 0 | 0 | 7 | 549.6839 | 0.716846 |
| 3632 | −0.35731 | 0 | 0 | 0.283735 | 0 | 0 | 7 | 547.8964 | 0.723257 |
| 3633 | −0.34421 | 0 | 0 | 0 | 0.260301 | 0 | 7 | 548.7098 | 0.72197 |
| 3634 | −0.34349 | 0 | 0 | 0 | 0 | 0.25144 | 7 | 546.9646 | 0.723257 |
| 3635 | −0.3635 | 0 | 0.280897 | 0.247949 | 0 | 0 | 7 | 541.7787 | 0.730667 |
| 3636 | −0.36484 | −0.51223 | 0.448932 | 0 | 0 | 0 | 7 | 536.504 | 0.735342 |
| 3637 | −0.36535 | −0.34122 | 0 | 0.272545 | 0 | 0 | 7 | 548.1985 | 0.718786 |
| 3638 | −0.35247 | −0.33749 | 0 | 0 | 0.244058 | 0 | 7 | 549.1416 | 0.720031 |
| 3639 | −0.3509 | −0.33426 | 0 | 0 | 0 | 0.230316 | 7 | 548.7851 | 0.717112 |
| 3640 | −0.35435 | 0 | 0.326983 | 0 | 0.276248 | 0 | 7 | 540.8678 | 0.731055 |
| 3641 | −0.35429 | 0 | 0.335115 | 0 | 0 | 0.278056 | 7 | 539.5674 | 0.733219 |
| 3642 | −0.35918 | 0 | 0 | 0.257296 | 0.231108 | 0 | 7 | 547.9335 | 0.720541 |
| 3643 | −0.36046 | 0 | 0 | 0.27483 | 0 | 0.241127 | 7 | 546.7248 | 0.721113 |
| 3644 | −0.34604 | 0 | 0 | 0 | 0.20186 | 0.189915 | 7 | 549.4107 | 0.718296 |
| 3645 | −0.3635 | 0 | 0.280897 | 0.247949 | 0 | 0 | 7 | 539.747 | 0.734587 |
| 3646 | −0.36484 | −0.51223 | 0.448932 | 0 | 0 | 0 | 7 | 534.2938 | 0.738915 |
| 3647 | −0.36535 | −0.34122 | 0 | 0.272545 | 0 | 0 | 7 | 545.3042 | 0.723481 |
| 3648 | −0.35247 | −0.33749 | 0 | 0 | 0.244058 | 0 | 7 | 545.9688 | 0.724563 |
| 3649 | −0.3509 | −0.33426 | 0 | 0 | 0 | 0.230316 | 7 | 544.6326 | 0.724216 |
| 3650 | −0.35435 | 0 | 0.326983 | 0 | 0.276248 | 0 | 7 | 538.4214 | 0.733382 |
| 3651 | −0.35429 | 0 | 0.335115 | 0 | 0 | 0.278056 | 7 | 535.9213 | 0.738874 |
| 3652 | −0.35918 | 0 | 0 | 0.257296 | 0.231108 | 0 | 7 | 544.9134 | 0.724747 |
| 3653 | −0.36046 | 0 | 0 | 0.27483 | 0 | 0.241127 | 7 | 542.5451 | 0.729912 |
| 3654 | −0.34604 | 0 | 0 | 0 | 0.20186 | 0.189915 | 7 | 545.4935 | 0.724971 |
| 3655 | −0.37665 | −0.49016 | 0.415897 | 0.213769 | 0 | 0 | 7 | 534.2989 | 0.738711 |
| 3656 | −0.3692 | −0.49878 | 0.458935 | 0 | 0.257995 | 0 | 7 | 532.8021 | 0.739691 |
| 3657 | −0.36789 | −0.4958 | 0.464972 | 0 | 0 | 0.255069 | 7 | 531.9802 | 0.742834 |
| 3658 | −0.36683 | −0.32775 | 0 | 0.247282 | 0.215748 | 0 | 7 | 545.9873 | 0.723481 |
| 3659 | −0.36769 | −0.32552 | 0 | 0.266766 | 0 | 0.223296 | 7 | 545.1098 | 0.721828 |
| 3660 | −0.35346 | −0.32601 | 0 | 0 | 0.190682 | 0.171912 | 7 | 547.5339 | 0.719602 |
| 3661 | −0.36573 | 0 | 0.297911 | 0.217151 | 0.249518 | 0 | 7 | 538.7221 | 0.733505 |
| 3662 | −0.36751 | 0 | 0.304766 | 0.234822 | 0 | 0.26618 | 7 | 537.1157 | 0.736404 |
| 3663 | −0.35702 | 0 | 0.341819 | 0 | 0.21209 | 0.213821 | 7 | 537.9835 | 0.734015 |
| 3664 | −0.36116 | 0 | 0 | 0.256551 | 0.172681 | 0.188346 | 7 | 546.1066 | 0.721603 |
| 3665 | −0.34954 | 0 | 0.311734 | 0 | 0 | 0 | 7 | 577.6287 | 0.665728 |
| 3666 | −0.34838 | −0.35299 | 0 | 0 | 0 | 0 | 7 | 576.614 | 0.667136 |
| 3667 | −0.35731 | 0 | 0 | 0.283735 | 0 | 0 | 7 | 578.5064 | 0.663604 |
| 3668 | −0.34421 | 0 | 0 | 0 | 0.260301 | 0 | 7 | 578.042 | 0.667014 |
| 3669 | −0.34349 | 0 | 0 | 0 | 0 | 0.25144 | 7 | 577.4248 | 0.666095 |
| 3670 | −0.3635 | 0 | 0.280897 | 0.247949 | 0 | 0 | 7 | 574.2068 | 0.672791 |
| 3671 | −0.36484 | −0.51223 | 0.448932 | 0 | 0 | 0 | 7 | 563.2305 | 0.692001 |
| 3672 | −0.36535 | −0.34122 | 0 | 0.272545 | 0 | 0 | 7 | 573.0242 | 0.676915 |
| 3673 | −0.35247 | −0.33749 | 0 | 0 | 0.244058 | 0 | 7 | 573.0557 | 0.682386 |
| 3674 | −0.3509 | −0.33426 | 0 | 0 | 0 | 0.230316 | 7 | 574.1509 | 0.671382 |
| 3675 | −0.35435 | 0 | 0.326983 | 0 | 0.276248 | 0 | 7 | 571.3256 | 0.679487 |
| 3676 | −0.35429 | 0 | 0.335115 | 0 | 0 | 0.278056 | 7 | 572.0293 | 0.674771 |
| 3677 | −0.35918 | 0 | 0 | 0.257296 | 0.231108 | 0 | 7 | 575.2446 | 0.67175 |
| 3678 | −0.36046 | 0 | 0 | 0.27483 | 0 | 0.241127 | 7 | 575.2698 | 0.666585 |
| 3679 | −0.34604 | 0 | 0 | 0 | 0.20186 | 0.189915 | 7 | 577.7032 | 0.668974 |
| 3680 | −0.3635 | 0 | 0.280897 | 0.247949 | 0 | 0 | 7 | 572.5319 | 0.675506 |
| 3681 | −0.36484 | −0.51223 | 0.448932 | 0 | 0 | 0 | 7 | 561.2577 | 0.696595 |
| 3682 | −0.36535 | −0.34122 | 0 | 0.272545 | 0 | 0 | 7 | 570.3624 | 0.683019 |
| 3683 | −0.35247 | −0.33749 | 0 | 0 | 0.244058 | 0 | 7 | 570.1905 | 0.683427 |
| 3684 | −0.3509 | −0.33426 | 0 | 0 | 0 | 0.230316 | 7 | 569.8714 | 0.67965 |
| 3685 | −0.35435 | 0 | 0.326983 | 0 | 0.276248 | 0 | 7 | 569.3614 | 0.678936 |
| 3686 | −0.35429 | 0 | 0.335115 | 0 | 0 | 0.278056 | 7 | 568.4184 | 0.681182 |
| 3687 | −0.35918 | 0 | 0 | 0.257296 | 0.231108 | 0 | 7 | 572.6592 | 0.674526 |
| 3688 | −0.36046 | 0 | 0 | 0.27483 | 0 | 0.241127 | 7 | 571.1224 | 0.675813 |
| 3689 | −0.34604 | 0 | 0 | 0 | 0.20186 | 0.189915 | 7 | 573.8835 | 0.672709 |
| 3690 | −0.37665 | −0.49016 | 0.415897 | 0.213769 | 0 | 0 | 7 | 559.5985 | 0.697718 |
| 3691 | −0.3692 | −0.49878 | 0.458935 | 0 | 0.257995 | 0 | 7 | 556.2649 | 0.70474 |
| 3692 | −0.36789 | −0.4958 | 0.464972 | 0 | 0 | 0.255069 | 7 | 557.2498 | 0.697391 |
| 3693 | −0.36683 | −0.32775 | 0 | 0.247282 | 0.215748 | 0 | 7 | 568.3855 | 0.687653 |
| 3694 | −0.36769 | −0.32552 | 0 | 0.266766 | 0 | 0.223296 | 7 | 568.6303 | 0.680957 |
| 3695 | −0.35346 | −0.32601 | 0 | 0 | 0.190682 | 0.171912 | 7 | 571.0047 | 0.679773 |
| 3696 | −0.36573 | 0 | 0.297911 | 0.217151 | 0.249518 | 0 | 7 | 567.7784 | 0.685795 |
| 3697 | −0.36751 | 0 | 0.304766 | 0.234822 | 0 | 0.26618 | 7 | 567.7814 | 0.682059 |
| 3698 | −0.35702 | 0 | 0.341819 | 0 | 0.21209 | 0.213821 | 7 | 567.8049 | 0.68357 |
| 3699 | −0.36116 | 0 | 0 | 0.256551 | 0.172681 | 0.188346 | 7 | 572.7658 | 0.673771 |
| 3700 | −0.3635 | 0 | 0.280897 | 0.247949 | 0 | 0 | 7 | 572.3137 | 0.675506 |
| 3701 | −0.36484 | −0.51223 | 0.448932 | 0 | 0 | 0 | 7 | 561.2423 | 0.696574 |
| 3702 | −0.36535 | −0.34122 | 0 | 0.272545 | 0 | 0 | 7 | 570.0758 | 0.683529 |
| 3703 | −0.35247 | −0.33749 | 0 | 0 | 0.244058 | 0 | 7 | 570.2765 | 0.686183 |
| 3704 | −0.3509 | −0.33426 | 0 | 0 | 0 | 0.230316 | 7 | 569.5028 | 0.682182 |
| 3705 | −0.35435 | 0 | 0.326983 | 0 | 0.276248 | 0 | 7 | 569.7189 | 0.677527 |
| 3706 | −0.35429 | 0 | 0.335115 | 0 | 0 | 0.278056 | 7 | 568.1745 | 0.682611 |
| 3707 | −0.35918 | 0 | 0 | 0.257296 | 0.231108 | 0 | 7 | 572.7368 | 0.674077 |
| 3708 | −0.36046 | 0 | 0 | 0.27483 | 0 | 0.241127 | 7 | 570.7005 | 0.676915 |
| 3709 | −0.34604 | 0 | 0 | 0 | 0.20186 | 0.189915 | 7 | 573.7828 | 0.672648 |
| 3710 | −0.37665 | −0.49016 | 0.415897 | 0.213769 | 0 | 0 | 7 | 559.8731 | 0.697248 |
| 3711 | −0.3692 | −0.49878 | 0.458935 | 0 | 0.257995 | 0 | 7 | 557.3784 | 0.701086 |
| 3712 | −0.36789 | −0.4958 | 0.464972 | 0 | 0 | 0.255069 | 7 | 557.6376 | 0.697228 |
| 3713 | −0.36683 | −0.32775 | 0 | 0.247282 | 0.215748 | 0 | 7 | 568.9359 | 0.687286 |
| 3714 | −0.36769 | −0.32552 | 0 | 0.266766 | 0 | 0.223296 | 7 | 568.6164 | 0.679773 |
| 3715 | −0.35346 | −0.32601 | 0 | 0 | 0.190682 | 0.171912 | 7 | 571.3959 | 0.67961 |
| 3716 | −0.36573 | 0 | 0.297911 | 0.217151 | 0.249518 | 0 | 7 | 568.6395 | 0.682733 |
| 3717 | −0.36751 | 0 | 0.304766 | 0.234822 | 0 | 0.26618 | 7 | 567.9661 | 0.680161 |
| 3718 | −0.35702 | 0 | 0.341819 | 0 | 0.21209 | 0.213821 | 7 | 568.6187 | 0.680079 |
| 3719 | −0.36116 | 0 | 0 | 0.256551 | 0.172681 | 0.188346 | 7 | 573.2047 | 0.669892 |
| 3720 | −0.37665 | −0.49016 | 0.415897 | 0.213769 | 0 | 0 | 7 | 557.4117 | 0.705047 |
| 3721 | −0.3692 | −0.49878 | 0.458935 | 0 | 0.257995 | 0 | 7 | 554.4406 | 0.70621 |
| 3722 | −0.36789 | −0.4958 | 0.464972 | 0 | 0 | 0.255069 | 7 | 553.1623 | 0.705986 |
| 3723 | −0.36683 | −0.32775 | 0 | 0.247282 | 0.215748 | 0 | 7 | 565.3574 | 0.691838 |
| 3724 | −0.36769 | −0.32552 | 0 | 0.266766 | 0 | 0.223296 | 7 | 563.536 | 0.692165 |
| 3725 | −0.35346 | −0.32601 | 0 | 0 | 0.190682 | 0.171912 | 7 | 566.6447 | 0.687776 |
| 3726 | −0.36573 | 0 | 0.297911 | 0.217151 | 0.249518 | 0 | 7 | 566.0157 | 0.684223 |
| 3727 | −0.36751 | 0 | 0.304766 | 0.234822 | 0 | 0.26618 | 7 | 563.6835 | 0.689123 |
| 3728 | −0.35702 | 0 | 0.341819 | 0 | 0.21209 | 0.213821 | 7 | 564.4465 | 0.686837 |
| 3729 | −0.36116 | 0 | 0 | 0.256551 | 0.172681 | 0.188346 | 7 | 568.5038 | 0.679058 |
| 3730 | 0 | 0 | 0 | 0 | 0 | 0 | 7 | 557.9597 | 0.706435 |
| 3731 | 0 | 0 | 0 | 0 | 0 | 0 | 7 | 514.4514 | 0.772334 |
| 3732 | 0 | 0 | 0 | 0 | 0 | 0 | 7 | 511.5493 | 0.777356 |
| 3733 | 0 | 0 | 0 | 0 | 0 | 0 | 7 | 511.1939 | 0.776886 |
| 3734 | 0 | 0 | 0 | 0 | 0 | 0 | 7 | 510.6459 | 0.777621 |
| 3735 | 0 | 0 | 0 | 0 | 0 | 0 | 7 | 513.231 | 0.774947 |
| 3736 | 0 | 0 | 0 | 0 | 0 | 0 | 7 | 568.3453 | 0.683856 |
| 3737 | −0.3784 | −0.48038 | 0.429094 | 0.184036 | 0.235336 | 0 | 7 | 554.9552 | 0.70425 |
| 3738 | −0.37944 | −0.47686 | 0.432955 | 0.204998 | 0 | 0.247828 | 7 | 554.5518 | 0.700841 |
| 3739 | −0.37057 | −0.48826 | 0.469023 | 0 | 0.198485 | 0.194003 | 7 | 554.5206 | 0.700821 |
| 3740 | −0.36821 | −0.31823 | 0 | 0.248938 | 0.16128 | 0.17389 | 7 | 566.969 | 0.683652 |
| 3741 | −0.36844 | 0 | 0.31303 | 0.214129 | 0.186465 | 0.210249 | 7 | 565.4006 | 0.686163 |
| 3742 | −0.34954 | 0 | 0.311734 | 0 | 0 | 0 | 6 | 546.5908 | 0.719602 |
| 3743 | −0.34838 | −0.35299 | 0 | 0 | 0 | 0 | 6 | 535.0357 | 0.741916 |
| 3744 | −0.35731 | 0 | 0 | 0.283735 | 0 | 0 | 6 | 543.9368 | 0.728238 |
| 3745 | −0.34421 | 0 | 0 | 0 | 0.260301 | 0 | 6 | 542.7235 | 0.72738 |
| 3746 | −0.34349 | 0 | 0 | 0 | 0 | 0.25144 | 6 | 543.8954 | 0.727442 |
| 3747 | −0.34954 | 0 | 0.311734 | 0 | 0 | 0 | 6 | 550.7641 | 0.710375 |
| 3748 | −0.34838 | −0.35299 | 0 | 0 | 0 | 0 | 6 | 539.0824 | 0.733627 |
| 3749 | −0.35731 | 0 | 0 | 0.283735 | 0 | 0 | 6 | 547.5136 | 0.719398 |
| 3750 | −0.34421 | 0 | 0 | 0 | 0.260301 | 0 | 6 | 546.9491 | 0.7185 |
| 3751 | −0.34349 | 0 | 0 | 0 | 0 | 0.25144 | 6 | 547.6877 | 0.718173 |
| 3752 | −0.34954 | 0 | 0.311734 | 0 | 0 | 0 | 6 | 550.9082 | 0.711947 |
| 3753 | −0.34838 | −0.35299 | 0 | 0 | 0 | 0 | 6 | 539.189 | 0.733811 |
| 3754 | −0.35731 | 0 | 0 | 0.283735 | 0 | 0 | 6 | 547.3212 | 0.721011 |
| 3755 | −0.34421 | 0 | 0 | 0 | 0.260301 | 0 | 6 | 546.2783 | 0.720991 |
| 3756 | −0.34349 | 0 | 0 | 0 | 0 | 0.25144 | 6 | 547.3701 | 0.719337 |
| 3757 | −0.34954 | 0 | 0.311734 | 0 | 0 | 0 | 6 | 550.8931 | 0.710028 |
| 3758 | −0.34838 | −0.35299 | 0 | 0 | 0 | 0 | 6 | 539.1942 | 0.73377 |
| 3759 | −0.35731 | 0 | 0 | 0.283735 | 0 | 0 | 6 | 547.3379 | 0.719214 |
| 3760 | −0.34421 | 0 | 0 | 0 | 0.260301 | 0 | 6 | 546.7958 | 0.718112 |
| 3761 | −0.34349 | 0 | 0 | 0 | 0 | 0.25144 | 6 | 547.3399 | 0.716928 |
| 3762 | −0.34954 | 0 | 0.311734 | 0 | 0 | 0 | 6 | 548.2006 | 0.71556 |
| 3763 | −0.34838 | −0.35299 | 0 | 0 | 0 | 0 | 6 | 535.254 | 0.740405 |
| 3764 | −0.35731 | 0 | 0 | 0.283735 | 0 | 0 | 6 | 543.9062 | 0.725992 |
| 3765 | −0.34421 | 0 | 0 | 0 | 0.260301 | 0 | 6 | 543.0366 | 0.724216 |
| 3766 | −0.34349 | 0 | 0 | 0 | 0 | 0.25144 | 6 | 542.2978 | 0.726972 |
| 3767 | −0.3635 | 0 | 0.280897 | 0.247949 | 0 | 0 | 6 | 547.0602 | 0.718867 |
| 3768 | −0.36484 | −0.51223 | 0.448932 | 0 | 0 | 0 | 6 | 532.5787 | 0.742855 |
| 3769 | −0.36535 | −0.34122 | 0 | 0.272545 | 0 | 0 | 6 | 534.8806 | 0.741487 |
| 3770 | −0.35247 | −0.33749 | 0 | 0 | 0.244058 | 0 | 6 | 534.7872 | 0.738445 |
| 3771 | −0.3509 | −0.33426 | 0 | 0 | 0 | 0.230316 | 6 | 535.5809 | 0.738874 |
| 3772 | −0.35435 | 0 | 0.326983 | 0 | 0.276248 | 0 | 6 | 545.3127 | 0.720092 |
| 3773 | −0.35429 | 0 | 0.335115 | 0 | 0 | 0.278056 | 6 | 545.8866 | 0.720154 |
| 3774 | −0.35918 | 0 | 0 | 0.257296 | 0.231108 | 0 | 6 | 543.1831 | 0.725584 |
| 3775 | −0.36046 | 0 | 0 | 0.27483 | 0 | 0.241127 | 6 | 543.1249 | 0.727585 |
| 3776 | −0.34604 | 0 | 0 | 0 | 0.20186 | 0.189915 | 6 | 544.7217 | 0.722787 |
| 3777 | −0.34954 | 0 | 0.311734 | 0 | 0 | 0 | 6 | 575.3347 | 0.661706 |
| 3778 | −0.34838 | −0.35299 | 0 | 0 | 0 | 0 | 6 | 576.2008 | 0.658154 |
| 3779 | −0.35731 | 0 | 0 | 0.283735 | 0 | 0 | 6 | 578.3819 | 0.658337 |
| 3780 | −0.34421 | 0 | 0 | 0 | 0.260301 | 0 | 6 | 578.297 | 0.66291 |
| 3781 | −0.34349 | 0 | 0 | 0 | 0 | 0.25144 | 6 | 579.3816 | 0.654642 |
| 3782 | −0.34954 | 0 | 0.311734 | 0 | 0 | 0 | 6 | 575.5036 | 0.662277 |
| 3783 | −0.34838 | −0.35299 | 0 | 0 | 0 | 0 | 6 | 576.4068 | 0.659052 |
| 3784 | −0.35731 | 0 | 0 | 0.283735 | 0 | 0 | 6 | 578.2894 | 0.657541 |
| 3785 | −0.34421 | 0 | 0 | 0 | 0.260301 | 0 | 6 | 577.6584 | 0.663788 |
| 3786 | −0.34349 | 0 | 0 | 0 | 0 | 0.25144 | 6 | 579.1719 | 0.654418 |
| 3787 | −0.34954 | 0 | 0.311734 | 0 | 0 | 0 | 6 | 574.9312 | 0.663951 |
| 3788 | −0.34838 | −0.35299 | 0 | 0 | 0 | 0 | 6 | 575.7496 | 0.662277 |
| 3789 | −0.35731 | 0 | 0 | 0.283735 | 0 | 0 | 6 | 577.6016 | 0.660603 |
| 3790 | −0.34421 | 0 | 0 | 0 | 0.260301 | 0 | 6 | 577.5059 | 0.666401 |
| 3791 | −0.34349 | 0 | 0 | 0 | 0 | 0.25144 | 6 | 578.3745 | 0.658725 |
| 3792 | −0.34954 | 0 | 0.311734 | 0 | 0 | 0 | 6 | 573.467 | 0.664605 |
| 3793 | −0.34838 | −0.35299 | 0 | 0 | 0 | 0 | 6 | 573.3045 | 0.665687 |
| 3794 | −0.35731 | 0 | 0 | 0.283735 | 0 | 0 | 6 | 575.4197 | 0.664217 |
| 3795 | −0.34421 | 0 | 0 | 0 | 0.260301 | 0 | 6 | 574.8276 | 0.665013 |
| 3796 | −0.34349 | 0 | 0 | 0 | 0 | 0.25144 | 6 | 574.3072 | 0.665217 |
| 3797 | −0.3635 | 0 | 0.280897 | 0.247949 | 0 | 0 | 6 | 570.836 | 0.673363 |
| 3798 | −0.36484 | −0.51223 | 0.448932 | 0 | 0 | 0 | 6 | 559.1201 | 0.691205 |
| 3799 | −0.36535 | −0.34122 | 0 | 0.272545 | 0 | 0 | 6 | 570.5195 | 0.672893 |
| 3800 | −0.35247 | −0.33749 | 0 | 0 | 0.244058 | 0 | 6 | 570.6638 | 0.676037 |
| 3801 | −0.3509 | −0.33426 | 0 | 0 | 0 | 0.230316 | 6 | 571.7817 | 0.667606 |
| 3802 | −0.35435 | 0 | 0.326983 | 0 | 0.276248 | 0 | 6 | 567.9629 | 0.676956 |
| 3803 | −0.35429 | 0 | 0.335115 | 0 | 0 | 0.278056 | 6 | 568.7557 | 0.675813 |
| 3804 | −0.35918 | 0 | 0 | 0.257296 | 0.231108 | 0 | 6 | 573.1517 | 0.66979 |
| 3805 | −0.36046 | 0 | 0 | 0.27483 | 0 | 0.241127 | 6 | 573.2608 | 0.665503 |
| 3806 | −0.34604 | 0 | 0 | 0 | 0.20186 | 0.189915 | 6 | 575.6521 | 0.66287 |
| 3807 | −0.34954 | 0 | 0.311734 | 0 | 0 | 0 | 6 | 576.863 | 0.659113 |
| 3808 | −0.34838 | −0.35299 | 0 | 0 | 0 | 0 | 6 | 576.9446 | 0.65795 |
| 3809 | −0.35731 | 0 | 0 | 0.283735 | 0 | 0 | 6 | 579.0869 | 0.657419 |
| 3810 | −0.34421 | 0 | 0 | 0 | 0.260301 | 0 | 6 | 578.6577 | 0.66487 |
| 3811 | −0.34349 | 0 | 0 | 0 | 0 | 0.25144 | 6 | 580.0104 | 0.655071 |
| 3812 | −0.34954 | 0 | 0.311734 | 0 | 0 | 0 | 6 | 576.7329 | 0.661338 |
| 3813 | −0.34838 | −0.35299 | 0 | 0 | 0 | 0 | 6 | 576.6401 | 0.660338 |
| 3814 | −0.35731 | 0 | 0 | 0.283735 | 0 | 0 | 6 | 578.7738 | 0.657174 |
| 3815 | −0.34421 | 0 | 0 | 0 | 0.260301 | 0 | 6 | 579.0621 | 0.665033 |
| 3816 | −0.34349 | 0 | 0 | 0 | 0 | 0.25144 | 6 | 579.6339 | 0.656582 |
| 3817 | −0.34954 | 0 | 0.311734 | 0 | 0 | 0 | 6 | 574.7163 | 0.664564 |
| 3818 | −0.34838 | −0.35299 | 0 | 0 | 0 | 0 | 6 | 573.6568 | 0.664829 |
| 3819 | −0.35731 | 0 | 0 | 0.283735 | 0 | 0 | 6 | 576.1524 | 0.663911 |
| 3820 | −0.34421 | 0 | 0 | 0 | 0.260301 | 0 | 6 | 575.8867 | 0.663931 |
| 3821 | −0.34349 | 0 | 0 | 0 | 0 | 0.25144 | 6 | 575.0949 | 0.664013 |
| 3822 | −0.3635 | 0 | 0.280897 | 0.247949 | 0 | 0 | 6 | 572.4443 | 0.669729 |
| 3823 | −0.36484 | −0.51223 | 0.448932 | 0 | 0 | 0 | 6 | 560.8504 | 0.690266 |
| 3824 | −0.36535 | −0.34122 | 0 | 0.272545 | 0 | 0 | 6 | 571.3817 | 0.67371 |
| 3825 | −0.35247 | −0.33749 | 0 | 0 | 0.244058 | 0 | 6 | 571.8823 | 0.674935 |
| 3826 | −0.3509 | −0.33426 | 0 | 0 | 0 | 0.230316 | 6 | 572.6998 | 0.666728 |
| 3827 | −0.35435 | 0 | 0.326983 | 0 | 0.276248 | 0 | 6 | 570.2326 | 0.676282 |
| 3828 | −0.35429 | 0 | 0.335115 | 0 | 0 | 0.278056 | 6 | 570.6111 | 0.671301 |
| 3829 | −0.35918 | 0 | 0 | 0.257296 | 0.231108 | 0 | 6 | 574.4946 | 0.6673 |
| 3830 | −0.36046 | 0 | 0 | 0.27483 | 0 | 0.241127 | 6 | 574.3201 | 0.661788 |
| 3831 | −0.34604 | 0 | 0 | 0 | 0.20186 | 0.189915 | 6 | 576.9598 | 0.664013 |
| 3832 | −0.34954 | 0 | 0.311734 | 0 | 0 | 0 | 6 | 506.6101 | 0.775437 |
| 3833 | −0.34838 | −0.35299 | 0 | 0 | 0 | 0 | 6 | 502.5484 | 0.778683 |
| 3834 | −0.35731 | 0 | 0 | 0.283735 | 0 | 0 | 6 | 506.3808 | 0.774192 |
| 3835 | −0.34421 | 0 | 0 | 0 | 0.260301 | 0 | 6 | 506.9689 | 0.773008 |
| 3836 | −0.34349 | 0 | 0 | 0 | 0 | 0.25144 | 6 | 506.4661 | 0.774845 |
| 3837 | −0.34954 | 0 | 0.311734 | 0 | 0 | 0 | 6 | 542.0693 | 0.726503 |
| 3838 | −0.34838 | −0.35299 | 0 | 0 | 0 | 0 | 6 | 550.3556 | 0.711334 |
| 3839 | −0.35731 | 0 | 0 | 0.283735 | 0 | 0 | 6 | 548.7108 | 0.715519 |
| 3840 | −0.34421 | 0 | 0 | 0 | 0.260301 | 0 | 6 | 549.4832 | 0.71852 |
| 3841 | −0.34349 | 0 | 0 | 0 | 0 | 0.25144 | 6 | 549.2474 | 0.716213 |
| 3842 | −0.34954 | 0 | 0.311734 | 0 | 0 | 0 | 6 | 543.1192 | 0.724726 |
| 3843 | −0.34838 | −0.35299 | 0 | 0 | 0 | 0 | 6 | 550.8364 | 0.710089 |
| 3844 | −0.35731 | 0 | 0 | 0.283735 | 0 | 0 | 6 | 549.1295 | 0.714642 |
| 3845 | −0.34421 | 0 | 0 | 0 | 0.260301 | 0 | 6 | 550.1056 | 0.71652 |
| 3846 | −0.34349 | 0 | 0 | 0 | 0 | 0.25144 | 6 | 549.6209 | 0.714049 |
| 3847 | −0.34954 | 0 | 0.311734 | 0 | 0 | 0 | 6 | 542.246 | 0.727687 |
| 3848 | −0.34838 | −0.35299 | 0 | 0 | 0 | 0 | 6 | 550.2205 | 0.712028 |
| 3849 | −0.35731 | 0 | 0 | 0.283735 | 0 | 0 | 6 | 548.3085 | 0.718092 |
| 3850 | −0.34421 | 0 | 0 | 0 | 0.260301 | 0 | 6 | 548.6353 | 0.718602 |
| 3851 | −0.34349 | 0 | 0 | 0 | 0 | 0.25144 | 6 | 548.7315 | 0.71556 |
| 3852 | −0.34954 | 0 | 0.311734 | 0 | 0 | 0 | 6 | 542.7959 | 0.725768 |
| 3853 | −0.34838 | −0.35299 | 0 | 0 | 0 | 0 | 6 | 550.3843 | 0.711641 |
| 3854 | −0.35731 | 0 | 0 | 0.283735 | 0 | 0 | 6 | 548.5776 | 0.716601 |
| 3855 | −0.34421 | 0 | 0 | 0 | 0.260301 | 0 | 6 | 549.6315 | 0.717581 |
| 3856 | −0.34349 | 0 | 0 | 0 | 0 | 0.25144 | 6 | 549.0248 | 0.715948 |
| 3857 | −0.34954 | 0 | 0.311734 | 0 | 0 | 0 | 6 | 541.0844 | 0.728646 |
| 3858 | −0.34838 | −0.35299 | 0 | 0 | 0 | 0 | 6 | 547.8424 | 0.715479 |
| 3859 | −0.35731 | 0 | 0 | 0.283735 | 0 | 0 | 6 | 546.1804 | 0.72097 |
| 3860 | −0.34421 | 0 | 0 | 0 | 0.260301 | 0 | 6 | 546.9151 | 0.719276 |
| 3861 | −0.34349 | 0 | 0 | 0 | 0 | 0.25144 | 6 | 545.3007 | 0.72295 |
| 3862 | −0.3635 | 0 | 0.280897 | 0.247949 | 0 | 0 | 6 | 539.9445 | 0.728871 |
| 3863 | −0.36484 | −0.51223 | 0.448932 | 0 | 0 | 0 | 6 | 534.4661 | 0.734689 |
| 3864 | −0.36535 | −0.34122 | 0 | 0.272545 | 0 | 0 | 6 | 546.4882 | 0.717398 |
| 3865 | −0.35247 | −0.33749 | 0 | 0 | 0.244058 | 0 | 6 | 547.3684 | 0.719459 |
| 3866 | −0.3509 | −0.33426 | 0 | 0 | 0 | 0.230316 | 6 | 547.1315 | 0.715499 |
| 3867 | −0.35435 | 0 | 0.326983 | 0 | 0.276248 | 0 | 6 | 538.935 | 0.730402 |
| 3868 | −0.35429 | 0 | 0.335115 | 0 | 0 | 0.278056 | 6 | 537.7786 | 0.732372 |
| 3869 | −0.35918 | 0 | 0 | 0.257296 | 0.231108 | 0 | 6 | 546.2786 | 0.719521 |
| 3870 | −0.36046 | 0 | 0 | 0.27483 | 0 | 0.241127 | 6 | 545.2248 | 0.7195 |
| 3871 | −0.34604 | 0 | 0 | 0 | 0.20186 | 0.189915 | 6 | 547.8009 | 0.717377 |
| 3872 | −0.34954 | 0 | 0.311734 | 0 | 0 | 0 | 6 | 577.2387 | 0.662033 |
| 3873 | −0.34838 | −0.35299 | 0 | 0 | 0 | 0 | 6 | 577.1417 | 0.660951 |
| 3874 | −0.35731 | 0 | 0 | 0.283735 | 0 | 0 | 6 | 578.8958 | 0.657296 |
| 3875 | −0.34421 | 0 | 0 | 0 | 0.260301 | 0 | 6 | 578.6473 | 0.666238 |
| 3876 | −0.34349 | 0 | 0 | 0 | 0 | 0.25144 | 6 | 579.6114 | 0.655459 |
| 3877 | −0.34954 | 0 | 0.311734 | 0 | 0 | 0 | 6 | 575.782 | 0.663645 |
| 3878 | −0.34838 | −0.35299 | 0 | 0 | 0 | 0 | 6 | 574.8046 | 0.665646 |
| 3879 | −0.35731 | 0 | 0 | 0.283735 | 0 | 0 | 6 | 576.8259 | 0.662584 |
| 3880 | −0.34421 | 0 | 0 | 0 | 0.260301 | 0 | 6 | 576.2985 | 0.663523 |
| 3881 | −0.34349 | 0 | 0 | 0 | 0 | 0.25144 | 6 | 575.8404 | 0.664952 |
| 3882 | −0.3635 | 0 | 0.280897 | 0.247949 | 0 | 0 | 6 | 572.4249 | 0.671566 |
| 3883 | −0.36484 | −0.51223 | 0.448932 | 0 | 0 | 0 | 6 | 561.2009 | 0.691267 |
| 3884 | −0.36535 | −0.34122 | 0 | 0.272545 | 0 | 0 | 6 | 571.3269 | 0.6752 |
| 3885 | −0.35247 | −0.33749 | 0 | 0 | 0.244058 | 0 | 6 | 571.2999 | 0.67863 |
| 3886 | −0.3509 | −0.33426 | 0 | 0 | 0 | 0.230316 | 6 | 572.5401 | 0.668341 |
| 3887 | −0.35435 | 0 | 0.326983 | 0 | 0.276248 | 0 | 6 | 569.4411 | 0.678303 |
| 3888 | −0.35429 | 0 | 0.335115 | 0 | 0 | 0.278056 | 6 | 570.312 | 0.673363 |
| 3889 | −0.35918 | 0 | 0 | 0.257296 | 0.231108 | 0 | 6 | 573.6116 | 0.670198 |
| 3890 | −0.36046 | 0 | 0 | 0.27483 | 0 | 0.241127 | 6 | 573.8145 | 0.665605 |
| 3891 | −0.34604 | 0 | 0 | 0 | 0.20186 | 0.189915 | 6 | 576.1601 | 0.666585 |
| 3892 | −0.34954 | 0 | 0.311734 | 0 | 0 | 0 | 6 | 575.4943 | 0.66436 |
| 3893 | −0.34838 | −0.35299 | 0 | 0 | 0 | 0 | 6 | 574.4443 | 0.667565 |
| 3894 | −0.35731 | 0 | 0 | 0.283735 | 0 | 0 | 6 | 576.3922 | 0.663257 |
| 3895 | −0.34421 | 0 | 0 | 0 | 0.260301 | 0 | 6 | 576.2605 | 0.666707 |
| 3896 | −0.34349 | 0 | 0 | 0 | 0 | 0.25144 | 6 | 575.3028 | 0.665666 |
| 3897 | −0.3635 | 0 | 0.280897 | 0.247949 | 0 | 0 | 6 | 572.3479 | 0.671199 |
| 3898 | −0.36484 | −0.51223 | 0.448932 | 0 | 0 | 0 | 6 | 561.342 | 0.690572 |
| 3899 | −0.36535 | −0.34122 | 0 | 0.272545 | 0 | 0 | 6 | 571.1381 | 0.676445 |
| 3900 | −0.35247 | −0.33749 | 0 | 0 | 0.244058 | 0 | 6 | 571.6784 | 0.679467 |
| 3901 | −0.3509 | −0.33426 | 0 | 0 | 0 | 0.230316 | 6 | 572.3019 | 0.670362 |
| 3902 | −0.35435 | 0 | 0.326983 | 0 | 0.276248 | 0 | 6 | 570.1907 | 0.677078 |
| 3903 | −0.35429 | 0 | 0.335115 | 0 | 0 | 0.278056 | 6 | 570.3163 | 0.673873 |
| 3904 | −0.35918 | 0 | 0 | 0.257296 | 0.231108 | 0 | 6 | 574.0258 | 0.670648 |
| 3905 | −0.36046 | 0 | 0 | 0.27483 | 0 | 0.241127 | 6 | 573.6166 | 0.664135 |
| 3906 | −0.34604 | 0 | 0 | 0 | 0.20186 | 0.189915 | 6 | 576.4001 | 0.665013 |
| 3907 | −0.3635 | 0 | 0.280897 | 0.247949 | 0 | 0 | 6 | 570.4702 | 0.6752 |
| 3908 | −0.36484 | −0.51223 | 0.448932 | 0 | 0 | 0 | 6 | 559.1754 | 0.696779 |
| 3909 | −0.36535 | −0.34122 | 0 | 0.272545 | 0 | 0 | 6 | 568.2714 | 0.682325 |
| 3910 | −0.35247 | −0.33749 | 0 | 0 | 0.244058 | 0 | 6 | 568.4297 | 0.683407 |
| 3911 | −0.3509 | −0.33426 | 0 | 0 | 0 | 0.230316 | 6 | 567.7602 | 0.679242 |
| 3912 | −0.35435 | 0 | 0.326983 | 0 | 0.276248 | 0 | 6 | 567.7961 | 0.677037 |
| 3913 | −0.35429 | 0 | 0.335115 | 0 | 0 | 0.278056 | 6 | 566.3726 | 0.679814 |
| 3914 | −0.35918 | 0 | 0 | 0.257296 | 0.231108 | 0 | 6 | 571.0136 | 0.671832 |
| 3915 | −0.36046 | 0 | 0 | 0.27483 | 0 | 0.241127 | 6 | 569.1103 | 0.675323 |
| 3916 | −0.34604 | 0 | 0 | 0 | 0.20186 | 0.189915 | 6 | 572.1237 | 0.669974 |
| 3917 | 0 | 0 | 0 | 0 | 0 | 0 | 6 | 560.3131 | 0.697534 |
| 3918 | 0 | 0 | 0 | 0 | 0 | 0 | 6 | 557.22 | 0.706251 |
| 3919 | 0 | 0 | 0 | 0 | 0 | 0 | 6 | 555.8605 | 0.706618 |
| 3920 | 0 | 0 | 0 | 0 | 0 | 0 | 6 | 556.9898 | 0.702719 |
| 3921 | 0 | 0 | 0 | 0 | 0 | 0 | 6 | 559.8062 | 0.695901 |
| 3922 | 0 | 0 | 0 | 0 | 0 | 0 | 6 | 604.0696 | 0.575984 |
| 3923 | 0 | 0 | 0 | 0 | 0 | 0 | 6 | 513.3432 | 0.771762 |
| 3924 | 0 | 0 | 0 | 0 | 0 | 0 | 6 | 513.4499 | 0.770762 |
| 3925 | 0 | 0 | 0 | 0 | 0 | 0 | 6 | 510.0435 | 0.776151 |
| 3926 | 0 | 0 | 0 | 0 | 0 | 0 | 6 | 512.2615 | 0.772334 |
| 3927 | 0 | 0 | 0 | 0 | 0 | 0 | 6 | 509.4095 | 0.776968 |
| 3928 | 0 | 0 | 0 | 0 | 0 | 0 | 6 | 509.0481 | 0.776315 |
| 3929 | 0 | 0 | 0 | 0 | 0 | 0 | 6 | 514.5697 | 0.768843 |
| 3930 | 0 | 0 | 0 | 0 | 0 | 0 | 6 | 511.7494 | 0.774049 |
| 3931 | 0 | 0 | 0 | 0 | 0 | 0 | 6 | 511.884 | 0.772293 |
| 3932 | 0 | 0 | 0 | 0 | 0 | 0 | 6 | 511.2686 | 0.774477 |
| 3933 | 0 | 0 | 0 | 0 | 0 | 0 | 6 | 568.7927 | 0.67077 |
| 3934 | 0 | 0 | 0 | 0 | 0 | 0 | 6 | 567.0542 | 0.677609 |
| 3935 | 0 | 0 | 0 | 0 | 0 | 0 | 6 | 566.4054 | 0.681345 |
| 3936 | 0 | 0 | 0 | 0 | 0 | 0 | 6 | 566.3047 | 0.680059 |
| 3937 | 0 | 0 | 0 | 0 | 0 | 0 | 6 | 566.4027 | 0.682754 |
| 3938 | −0.37665 | −0.49016 | 0.415897 | 0.213769 | 0 | 0 | 6 | 557.8754 | 0.696758 |
| 3939 | −0.3692 | −0.49878 | 0.458935 | 0 | 0.257995 | 0 | 6 | 555.3242 | 0.699963 |
| 3940 | −0.36789 | −0.4958 | 0.464972 | 0 | 0 | 0.255069 | 6 | 555.6732 | 0.695268 |
| 3941 | −0.36683 | −0.32775 | 0 | 0.247282 | 0.215748 | 0 | 6 | 567.2378 | 0.684244 |
| 3942 | −0.36769 | −0.32552 | 0 | 0.266766 | 0 | 0.223296 | 6 | 567.0482 | 0.677956 |
| 3943 | −0.35346 | −0.32601 | 0 | 0 | 0.190682 | 0.171912 | 6 | 569.7507 | 0.676629 |
| 3944 | −0.36573 | 0 | 0.297911 | 0.217151 | 0.249518 | 0 | 6 | 566.8072 | 0.682162 |
| 3945 | −0.36751 | 0 | 0.304766 | 0.234822 | 0 | 0.26618 | 6 | 566.2807 | 0.678813 |
| 3946 | −0.35702 | 0 | 0.341819 | 0 | 0.21209 | 0.213821 | 6 | 566.8356 | 0.678752 |
| 3947 | −0.36116 | 0 | 0 | 0.256551 | 0.172681 | 0.188346 | 6 | 571.7062 | 0.668443 |
| 3948 | −0.34954 | 0 | 0.311734 | 0 | 0 | 0 | 5 | 549.166 | 0.710763 |
| 3949 | −0.34838 | −0.35299 | 0 | 0 | 0 | 0 | 5 | 537.4593 | 0.733362 |
| 3950 | −0.35731 | 0 | 0 | 0.283735 | 0 | 0 | 5 | 545.6838 | 0.719847 |
| 3951 | −0.34421 | 0 | 0 | 0 | 0.260301 | 0 | 5 | 545.1371 | 0.718602 |
| 3952 | −0.34349 | 0 | 0 | 0 | 0 | 0.25144 | 5 | 545.7864 | 0.717214 |
| 3953 | −0.34954 | 0 | 0.311734 | 0 | 0 | 0 | 5 | 573.5256 | 0.660869 |
| 3954 | −0.34838 | −0.35299 | 0 | 0 | 0 | 0 | 5 | 574.3735 | 0.658378 |
| 3955 | −0.35731 | 0 | 0 | 0.283735 | 0 | 0 | 5 | 576.3741 | 0.657643 |
| 3956 | −0.34421 | 0 | 0 | 0 | 0.260301 | 0 | 5 | 576.2644 | 0.661951 |
| 3957 | −0.34349 | 0 | 0 | 0 | 0 | 0.25144 | 5 | 577.3128 | 0.653683 |
| 3958 | −0.34954 | 0 | 0.311734 | 0 | 0 | 0 | 5 | 575.1241 | 0.658317 |
| 3959 | −0.34838 | −0.35299 | 0 | 0 | 0 | 0 | 5 | 575.1145 | 0.657786 |
| 3960 | −0.35731 | 0 | 0 | 0.283735 | 0 | 0 | 5 | 577.3632 | 0.656418 |
| 3961 | −0.34421 | 0 | 0 | 0 | 0.260301 | 0 | 5 | 577.5857 | 0.660706 |
| 3962 | −0.34349 | 0 | 0 | 0 | 0 | 0.25144 | 5 | 578.3369 | 0.653764 |
| 3963 | −0.34954 | 0 | 0.311734 | 0 | 0 | 0 | 5 | 540.957 | 0.724818 |
| 3964 | −0.34838 | −0.35299 | 0 | 0 | 0 | 0 | 5 | 548.6741 | 0.710191 |
| 3965 | −0.35731 | 0 | 0 | 0.283735 | 0 | 0 | 5 | 546.9957 | 0.715131 |
| 3966 | −0.34421 | 0 | 0 | 0 | 0.260301 | 0 | 5 | 547.9671 | 0.716111 |
| 3967 | −0.34349 | 0 | 0 | 0 | 0 | 0.25144 | 5 | 547.5177 | 0.714182 |
| 3968 | −0.34954 | 0 | 0.311734 | 0 | 0 | 0 | 5 | 575.485 | 0.659746 |
| 3969 | −0.34838 | −0.35299 | 0 | 0 | 0 | 0 | 5 | 575.4662 | 0.659358 |
| 3970 | −0.35731 | 0 | 0 | 0.283735 | 0 | 0 | 5 | 577.3548 | 0.654887 |
| 3971 | −0.34421 | 0 | 0 | 0 | 0.260301 | 0 | 5 | 577.0369 | 0.663237 |
| 3972 | −0.34349 | 0 | 0 | 0 | 0 | 0.25144 | 5 | 578.1943 | 0.65305 |
| 3973 | −0.34954 | 0 | 0.311734 | 0 | 0 | 0 | 5 | 575.279 | 0.661502 |
| 3974 | −0.34838 | −0.35299 | 0 | 0 | 0 | 0 | 5 | 575.1251 | 0.660603 |
| 3975 | −0.35731 | 0 | 0 | 0.283735 | 0 | 0 | 5 | 576.9965 | 0.656296 |
| 3976 | −0.34421 | 0 | 0 | 0 | 0.260301 | 0 | 5 | 577.271 | 0.66242 |
| 3977 | −0.34349 | 0 | 0 | 0 | 0 | 0.25144 | 5 | 577.7706 | 0.654826 |
| 3978 | −0.34954 | 0 | 0.311734 | 0 | 0 | 0 | 5 | 573.6819 | 0.663135 |
| 3979 | −0.34838 | −0.35299 | 0 | 0 | 0 | 0 | 5 | 572.6681 | 0.665544 |
| 3980 | −0.35731 | 0 | 0 | 0.283735 | 0 | 0 | 5 | 574.7474 | 0.661706 |
| 3981 | −0.34421 | 0 | 0 | 0 | 0.260301 | 0 | 5 | 574.5617 | 0.662931 |
| 3982 | −0.34349 | 0 | 0 | 0 | 0 | 0.25144 | 5 | 573.7529 | 0.664196 |
| 3983 | 0 | 0 | 0 | 0 | 0 | 0 | 5 | 559.698 | 0.697901 |
| 3984 | 0 | 0 | 0 | 0 | 0 | 0 | 5 | 558.5047 | 0.696881 |
| 3985 | 0 | 0 | 0 | 0 | 0 | 0 | 5 | 555.1485 | 0.706006 |
| 3986 | 0 | 0 | 0 | 0 | 0 | 0 | 5 | 558.8074 | 0.694594 |
| 3987 | 0 | 0 | 0 | 0 | 0 | 0 | 5 | 555.9749 | 0.701903 |
| 3988 | 0 | 0 | 0 | 0 | 0 | 0 | 5 | 554.838 | 0.702597 |
| 3989 | 0 | 0 | 0 | 0 | 0 | 0 | 5 | 561.746 | 0.68653 |
| 3990 | 0 | 0 | 0 | 0 | 0 | 0 | 5 | 558.4783 | 0.697534 |
| 3991 | 0 | 0 | 0 | 0 | 0 | 0 | 5 | 557.8119 | 0.695125 |
| 3992 | 0 | 0 | 0 | 0 | 0 | 0 | 5 | 559.2279 | 0.690919 |
| 3993 | 0 | 0 | 0 | 0 | 0 | 0 | 5 | 605.427 | 0.549343 |
| 3994 | 0 | 0 | 0 | 0 | 0 | 0 | 5 | 603.1228 | 0.576862 |
| 3995 | 0 | 0 | 0 | 0 | 0 | 0 | 5 | 601.9106 | 0.5748 |
| 3996 | 0 | 0 | 0 | 0 | 0 | 0 | 5 | 603.0967 | 0.557386 |
| 3997 | 0 | 0 | 0 | 0 | 0 | 0 | 5 | 602.6545 | 0.574514 |
| 3998 | 0 | 0 | 0 | 0 | 0 | 0 | 5 | 512.4079 | 0.769476 |
| 3999 | 0 | 0 | 0 | 0 | 0 | 0 | 5 | 511.1832 | 0.771946 |
| 4000 | 0 | 0 | 0 | 0 | 0 | 0 | 5 | 511.3102 | 0.77068 |
| 4001 | 0 | 0 | 0 | 0 | 0 | 0 | 5 | 507.8896 | 0.775661 |
| 4002 | 0 | 0 | 0 | 0 | 0 | 0 | 5 | 513.1943 | 0.76774 |
| 4003 | 0 | 0 | 0 | 0 | 0 | 0 | 5 | 513.7874 | 0.765883 |
| 4004 | 0 | 0 | 0 | 0 | 0 | 0 | 5 | 510.4289 | 0.771762 |
| 4005 | 0 | 0 | 0 | 0 | 0 | 0 | 5 | 512.4419 | 0.76868 |
| 4006 | 0 | 0 | 0 | 0 | 0 | 0 | 5 | 509.7368 | 0.773722 |
| 4007 | 0 | 0 | 0 | 0 | 0 | 0 | 5 | 509.8421 | 0.773028 |
| 4008 | 0 | 0 | 0 | 0 | 0 | 0 | 5 | 567.5785 | 0.663278 |
| 4009 | 0 | 0 | 0 | 0 | 0 | 0 | 5 | 567.1163 | 0.666034 |
| 4010 | 0 | 0 | 0 | 0 | 0 | 0 | 5 | 565.1559 | 0.677037 |
| 4011 | 0 | 0 | 0 | 0 | 0 | 0 | 5 | 566.6406 | 0.670137 |
| 4012 | 0 | 0 | 0 | 0 | 0 | 0 | 5 | 564.9863 | 0.673342 |
| 4013 | 0 | 0 | 0 | 0 | 0 | 0 | 5 | 564.3336 | 0.677487 |
| 4014 | 0 | 0 | 0 | 0 | 0 | 0 | 5 | 566.8154 | 0.668382 |
| 4015 | 0 | 0 | 0 | 0 | 0 | 0 | 5 | 565.0303 | 0.676323 |
| 4016 | 0 | 0 | 0 | 0 | 0 | 0 | 5 | 564.5373 | 0.682264 |
| 4017 | 0 | 0 | 0 | 0 | 0 | 0 | 5 | 564.4451 | 0.67863 |
| 4018 | −0.3635 | 0 | 0.280897 | 0.247949 | 0 | 0 | 5 | 570.6137 | 0.669872 |
| 4019 | −0.35435 | 0 | 0.326983 | 0 | 0.276248 | 0 | 5 | 568.3687 | 0.675568 |
| 4020 | −0.35429 | 0 | 0.335115 | 0 | 0 | 0.278056 | 5 | 568.6481 | 0.671576 |
| 4021 | −0.35918 | 0 | 0 | 0.257296 | 0.231108 | 0 | 5 | 572.4569 | 0.667524 |
| 4022 | −0.36046 | 0 | 0 | 0.27483 | 0 | 0.241127 | 5 | 572.2133 | 0.662155 |
| 4023 | −0.34604 | 0 | 0 | 0 | 0.20186 | 0.189915 | 5 | 574.9182 | 0.662767 |
| 4024 | −0.36484 | −0.51223 | 0.448932 | 0 | 0 | 0 | 5 | 559.3585 | 0.689633 |
| 4025 | −0.36535 | −0.34122 | 0 | 0.272545 | 0 | 0 | 5 | 569.4913 | 0.674363 |
| 4026 | −0.35247 | −0.33749 | 0 | 0 | 0.244058 | 0 | 5 | 569.9841 | 0.675976 |
| 4027 | −0.3509 | −0.33426 | 0 | 0 | 0 | 0.230316 | 5 | 570.7429 | 0.667381 |
| 4028 | 0 | 0 | 0 | 0 | 0 | 0 | 4 | 557.9168 | 0.696391 |
| 4029 | 0 | 0 | 0 | 0 | 0 | 0 | 4 | 557.992 | 0.694941 |
| 4030 | 0 | 0 | 0 | 0 | 0 | 0 | 4 | 556.8607 | 0.694308 |
| 4031 | 0 | 0 | 0 | 0 | 0 | 0 | 4 | 553.8571 | 0.701821 |
| 4032 | 0 | 0 | 0 | 0 | 0 | 0 | 4 | 560.5582 | 0.687224 |
| 4033 | 0 | 0 | 0 | 0 | 0 | 0 | 4 | 560.0595 | 0.685979 |
| 4034 | 0 | 0 | 0 | 0 | 0 | 0 | 4 | 556.4999 | 0.696697 |
| 4035 | 0 | 0 | 0 | 0 | 0 | 0 | 4 | 560.5402 | 0.682202 |
| 4036 | 0 | 0 | 0 | 0 | 0 | 0 | 4 | 557.6587 | 0.69145 |
| 4037 | 0 | 0 | 0 | 0 | 0 | 0 | 4 | 557.1294 | 0.690715 |
| 4038 | 0 | 0 | 0 | 0 | 0 | 0 | 4 | 604.6031 | 0.540666 |
| 4039 | 0 | 0 | 0 | 0 | 0 | 0 | 4 | 603.3629 | 0.548383 |
| 4040 | 0 | 0 | 0 | 0 | 0 | 0 | 4 | 600.9932 | 0.576882 |
| 4041 | 0 | 0 | 0 | 0 | 0 | 0 | 4 | 603.9142 | 0.537992 |
| 4042 | 0 | 0 | 0 | 0 | 0 | 0 | 4 | 601.9811 | 0.553364 |
| 4043 | 0 | 0 | 0 | 0 | 0 | 0 | 4 | 600.9843 | 0.558325 |
| 4044 | 0 | 0 | 0 | 0 | 0 | 0 | 4 | 603.9288 | 0.542197 |
| 4045 | 0 | 0 | 0 | 0 | 0 | 0 | 4 | 601.5145 | 0.576515 |
| 4046 | 0 | 0 | 0 | 0 | 0 | 0 | 4 | 600.5365 | 0.573003 |
| 4047 | 0 | 0 | 0 | 0 | 0 | 0 | 4 | 510.3528 | 0.769394 |
| 4048 | 0 | 0 | 0 | 0 | 0 | 0 | 4 | 512.4459 | 0.765046 |
| 4049 | 0 | 0 | 0 | 0 | 0 | 0 | 4 | 511.0672 | 0.767659 |
| 4050 | 0 | 0 | 0 | 0 | 0 | 0 | 4 | 511.6529 | 0.765964 |
| 4051 | 0 | 0 | 0 | 0 | 0 | 0 | 4 | 508.3584 | 0.772171 |
| 4052 | 0 | 0 | 0 | 0 | 0 | 0 | 4 | 565.9511 | 0.664258 |
| 4053 | 0 | 0 | 0 | 0 | 0 | 0 | 4 | 565.4438 | 0.663257 |
| 4054 | 0 | 0 | 0 | 0 | 0 | 0 | 4 | 564.9829 | 0.665911 |
| 4055 | 0 | 0 | 0 | 0 | 0 | 0 | 4 | 563.0682 | 0.673404 |
| 4056 | 0 | 0 | 0 | 0 | 0 | 0 | 4 | 565.5347 | 0.659072 |
| 4057 | 0 | 0 | 0 | 0 | 0 | 0 | 4 | 565.2171 | 0.665483 |
| 4058 | 0 | 0 | 0 | 0 | 0 | 0 | 4 | 563.194 | 0.678058 |
| 4059 | 0 | 0 | 0 | 0 | 0 | 0 | 4 | 564.7077 | 0.667361 |
| 4060 | 0 | 0 | 0 | 0 | 0 | 0 | 4 | 563.0269 | 0.673179 |
| 4061 | 0 | 0 | 0 | 0 | 0 | 0 | 4 | 562.5348 | 0.678201 |
| 4062 | 0 | 0 | 0 | 0 | 0 | 0 | 4 | 601.9665 | 0.558141 |
| 4063 | −0.34838 | −0.35299 | 0 | 0 | 0 | 0 | 4 | 573.4927 | 0.65897 |
| 4064 | −0.34954 | 0 | 0.311734 | 0 | 0 | 0 | 4 | 573.5676 | 0.658276 |
| 4065 | −0.35731 | 0 | 0 | 0.283735 | 0 | 0 | 4 | 575.5014 | 0.655306 |
| 4066 | −0.34421 | 0 | 0 | 0 | 0.260301 | 0 | 4 | 575.7212 | 0.659664 |
| 4067 | −0.34349 | 0 | 0 | 0 | 0 | 0.25144 | 4 | 576.3998 | 0.649947 |
| 4068 | 0 | 0 | 0 | 0 | 0 | 0 | 3 | 556.1008 | 0.694798 |
| 4069 | 0 | 0 | 0 | 0 | 0 | 0 | 3 | 558.8825 | 0.685203 |
| 4070 | 0 | 0 | 0 | 0 | 0 | 0 | 3 | 559.1554 | 0.682631 |
| 4071 | 0 | 0 | 0 | 0 | 0 | 0 | 3 | 558.6608 | 0.682243 |
| 4072 | 0 | 0 | 0 | 0 | 0 | 0 | 3 | 555.5898 | 0.691573 |
| 4073 | 0 | 0 | 0 | 0 | 0 | 0 | 3 | 602.5694 | 0.539748 |
| 4074 | 0 | 0 | 0 | 0 | 0 | 0 | 3 | 602.9652 | 0.536236 |
| 4075 | 0 | 0 | 0 | 0 | 0 | 0 | 3 | 601.815 | 0.536563 |
| 4076 | 0 | 0 | 0 | 0 | 0 | 0 | 3 | 599.8905 | 0.553956 |
| 4077 | 0 | 0 | 0 | 0 | 0 | 0 | 3 | 602.9227 | 0.521395 |
| 4078 | 0 | 0 | 0 | 0 | 0 | 0 | 3 | 601.9214 | 0.537747 |
| 4079 | 0 | 0 | 0 | 0 | 0 | 0 | 3 | 599.4231 | 0.576106 |
| 4080 | 0 | 0 | 0 | 0 | 0 | 0 | 3 | 510.3565 | 0.76524 |
| 4081 | 0 | 0 | 0 | 0 | 0 | 0 | 3 | 563.8625 | 0.663094 |
| 4082 | 0 | 0 | 0 | 0 | 0 | 0 | 3 | 563.9696 | 0.659297 |
| 4083 | 0 | 0 | 0 | 0 | 0 | 0 | 3 | 563.432 | 0.658011 |
| 4084 | 0 | 0 | 0 | 0 | 0 | 0 | 3 | 563.1103 | 0.665176 |
| 4085 | 0 | 0 | 0 | 0 | 0 | 0 | 3 | 561.1601 | 0.673996 |
| 4086 | 0 | 0 | 0 | 0 | 0 | 0 | 3 | 602.6259 | 0.522375 |
| 4087 | 0 | 0 | 0 | 0 | 0 | 0 | 3 | 600.618 | 0.555998 |
| 4088 | 0 | 0 | 0 | 0 | 0 | 0 | 3 | 599.87 | 0.55757 |
| 4089 | 0 | 0 | 0 | 0 | 0 | 0 | 2 | 557.3154 | 0.681478 |
| 4090 | 0 | 0 | 0 | 0 | 0 | 0 | 2 | 600.8991 | 0.535542 |
| 4091 | 0 | 0 | 0 | 0 | 0 | 0 | 2 | 600.9393 | 0.512616 |
| 4092 | 0 | 0 | 0 | 0 | 0 | 0 | 2 | 561.8981 | 0.651968 |
| 4093 | 0 | 0 | 0 | 0 | 0 | 0 | 2 | 601.4543 | 0.507084 |
| 4094 | 0 | 0 | 0 | 0 | 0 | 0 | 2 | 600.5605 | 0.524171 |
| 4095 | 0 | 0 | 0 | 0 | 0 | 0 | 2 | 598.5467 | 0.557376 |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method for determining coronary artery disease risk in a subject, comprising:
performing or having performed at least one protein detection assay on a sample from the subject to generate a dataset comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6; and
generating or having generated, by a computer processor, a score indicative of coronary artery disease (CAD) risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD.

2. The method of claim 1, wherein the at least one protein detection assay is at least one enzyme-linked immunosorbent assay (ELISA), wherein the dataset comprises data representing expression levels corresponding to at least five markers comprising corn, APOB, HSP70, RBP4, SERPINA12, NTproBNP, PIGF, adiponectin, APOA1, S100A8, MPO, S100A12, or TNFAIP6, and wherein the score is more predictive of CAD than a score produced using Corus with the sample as measured using AIC or AUC.

3. The method of claim 1, wherein the dataset comprises data representing expression levels corresponding to at least three, four, or five markers comprising corin, APOB, HSP70, RBP4, SERPINA12, NTproBNP, PIGF, adiponectin, APOA1, S100A8, MPO, S100A12, or TNFAIP6.

4. The method of claim 1, wherein the dataset comprises data representing expression levels corresponding to at least three, four, or five markers comprising APOB, HSP70, SERPINA12, NTproBNP, PIGF, adiponectin, APOA1, S100A8, MPO, S100A12, or TNFAIP6.

5. The method of claim 1, further comprising classifying the sample according to the score.

6. The method of claim 1, further comprising rating CAD risk using the score.

7. The method of claim 1, wherein the sample comprises protein extracted from the blood of the subject.

8. The method of claim 1, wherein the mathematical combination is based on a predictive model, optionally wherein the predictive model is a partial least squares model, a logistic regression model, a linear regression model, a linear discriminant analysis model, a ridge regression model, or a tree-based recursive partitioning model.

9. The method of claim 1, wherein CAD is obstructive CAD.

10. (canceled)

11. The method of claim 1, wherein the method performance is characterized by an area under the curve (AUC) ranging of at least 0.5, 0.52, 0.6, 0.7, 0.8, or 0.81.

12. The method of claim 1, further comprising obtaining data representing at least one clinical factor associated with the subject, optionally wherein the clinical factor comprises age of the subject and/or gender of the subject, and optionally mathematically combining the data representing the at least one clinical factor with the data representing the protein expression levels to generate the score.

13. The method of claim 1, further comprising obtaining data representing at least one clinical factor associated with the subject, wherein the at least one clinical factor comprises at least one of age and gender.

14. (canceled)

15. The method of claim 12, wherein the method comprises mathematically combining the data representing the at least one clinical factor with the data representing the protein expression levels to generate the score.

16. The method of claim 1, wherein the subject is human.

17. The method of claim 1, wherein the at least one protein detection assay is an immunoassay, a protein-binding assay, an antibody-based assay, an antigen-binding protein-based assay, a protein-based array, an enzyme-linked immunosorbent assay (ELISA), flow cytometry, a protein array, a blot, a Western blot, nephelometry, turbidimetry, chromatography, mass spectrometry, enzymatic activity, and an immunoassays selected from RIA, immunofluorescence, immunochemiluminescence, immunoelectrochemiluminescence, immunoelectrophoretic, a competitive immunoassay, amd immunoprecipitation.

18. The method of claim 1, further comprising taking at least one action based on the score, optionally wherein the at least one action comprises treating the subject, advising lifestyle changes to the subject, performing a procedure on the subject, performing further diagnostics on the subject, assessing the subject's health further, optimizing medical therapy, investigating non-cardiac etiologies of symptoms, or performing angiography on the subject.

19. A method for determining coronary artery disease risk in a subject, comprising:
obtaining or having obtained a dataset associated with a sample from the subject comprising data representing protein expression levels to at least two markers comprising corin, APOB, HSP70, RBP4, SERPINA12, NTproBNP, PIGF, adiponectin, APOA1, S100A8, MPO, S100A12, or TNFAIP6;
generating or having generated, by a computer processor, a score indicative of coronary artery disease (CAD) risk by mathematically combining the data representing the protein expression levels, wherein a higher score relative to a control subject having less than 50% stenosis in all major vessels as measured using Quantitative Coronary Angiography (QCA) indicates an increased likelihood that the subject has CAD or a lower score relative to a control subject having greater than or equal to 50% stenosis in at least one major coronary vessel as measured using QCA indicates a decreased likelihood that the subject has CAD.

20. The method of claim 19, wherein the dataset comprises data representing expression levels corresponding to at least five markers comprising corin, APOB, HSP70, RBP4, SERPINA12, NTproBNP, PIGF, adiponectin, APOA1, S100A8, MPO, S100A12, or TNFAIP6, and wherein the score is more predictive of CAD than a score produced using Corus with the sample as measured using AIC or AUC.

21.-29. (canceled)

30. The method of claim 19, further comprising obtaining data representing at least one clinical factor associated with the subject, wherein the at least one clinical factor comprises at least one of age and gender.

31.-39. (canceled)

40. A method for generating a dataset comprising data representing protein expression levels for a subject that has CAD or is suspected of having CAD, comprising:
obtaining or having obtained a sample from the subject, wherein the subject has CAD or is suspected of having CAD;
performing or having performed at least one protein detection assay on the sample to generate a dataset comprising data representing protein expression levels corresponding to at least two markers comprising corin, APOB, HSP70, RBP4, SERPINA12, NTproBNP, PIGF, adiponectin, APOA1, S100A8, MPO, S100A12, or TNFAIP6.

41.-104. (canceled)
